(19) United States
(12) Patent Application Publication
(10) Pub. No.: US 2026/0356856 A1
(43) Pub. Date: Nov. 12, 2026

(54) KRAS G12S AND G12C INHIBITORS

(71) Applicant: MIRATI THERAPEUTICS, INC., PRINCETON, NJ (US)

(72) Inventors: XIAOLUN WANG, SAN DIEGO, CA (US); MATTHEW ARNOLD MARX, BONITA, CA (US); JOHN DAVID LAWSON, CARLSBAD, CA (US)

(73) Assignee: MIRATI THERAPEUTICS, INC., PRINCETON, NJ (US)

(21) Appl. No.: 19/484,410

(22) PCT Filed: May 15, 2024
(86) PCT No.: PCT/US2024/029427

§ 371 Date: Nov. 14, 2025

Related U.S. Application Data
(60) Provisional application No. 63/466,476, filed on May 15, 2023.
(60) Provisional application No. 63/626,329, filed on Jan. 29, 2024.

(51) Int. Cl. C07D 519/00; A61K 31/506; A61K 31/517; A61K 31/519; A61K 31/5377; A61K 31/5386; A61K 31/5513; A61K 31/553; C07D 409/14; C07D 413/14; C07D 417/14; C07D 471/04; C07D 487/04; C07D 498/10

(52) U.S. Cl. CPC C07D 519/00; A61K 31/506; A61K 31/517; A61K 31/519; A61K 31/5377; A61K 31/5386; A61K 31/5513; A61K 31/553; C07D 409/14; C07D 413/14; C07D 417/14; C07D 471/04; C07D 487/04; C07D 498/10

(57) ABSTRACT

The present invention relates to compounds that inhibit KRas G12S and/or KRas G12C. In particular, the present invention relates to compounds that inhibit the activity of KRas G12S and/or KRas G12C, pharmaceutical compositions comprising the compounds and methods of use therefor.

### FIELD OF THE INVENTION

The present invention relates to compounds that inhibit KRas G12S and/or KRas G12C. In particular, the present invention relates to compounds that inhibit the activity of KRas G12S and/or KRas G12C, pharmaceutical compositions comprising the compounds and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma. KRAS G12S mutations were present in 2% of colorectal carcinoma patients, 0.6% of ovarian cancer patients, 0.5% of non-small cell lung carcinomas patients, 0.2% of bladder and gastric cancers, and in 0.1% of pancreatic ductal adenocarcinoma, breast cancer, and endometrial cancer patients (The AACR Project GENIE Consortium. AACR Project GENIE: Powering Precision Medicine Through An International Consortium, Cancer Discovery 2017.Cohort v. 13.1-public).

The well-known role of KRas in malignancy and the discovery of these frequent mutations in KRas in various tumor types made KRas a highly attractive target of the pharmaceutical industry for cancer therapy. Notwithstanding thirty years of large-scale discovery efforts to develop inhibitors of KRas for treating cancer, very few KRas inhibitors have demonstrated sufficient safety and/or efficacy to obtain regulatory approval.

Compounds that inhibit KRas activity are still highly desirable and under investigation, including those that disrupt effectors such as guanine nucleotide exchange factors (e.g., see Sun et al., (2012) Agnew Chem Int Ed Engl. 51(25): 6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly there remains a continued interest and effort to develop inhibitors of KRas, particularly inhibitors of activating KRas mutants, including KRas G12S and KRas G12C.

Thus, there is a need to develop new KRas G12S and/or KRas G12C inhibitors that demonstrate sufficient efficacy for treating KRas G12S-mediated cancer and/or KRas G12C-mediated cancer.

### SUMMARY OF THE INVENTION

In one aspect of the invention, compounds are provided that inhibit KRas G12S and/or KRas G12C activity.

In certain embodiments, the compounds are represented by Formula (Ia) or (Ib):

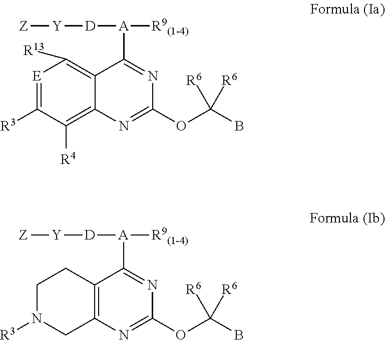

or a pharmaceutically acceptable salt thereof, wherein:

A is selected from:

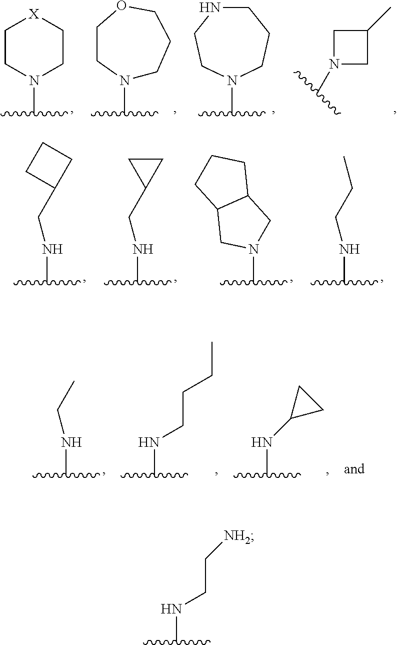

B is selected from:

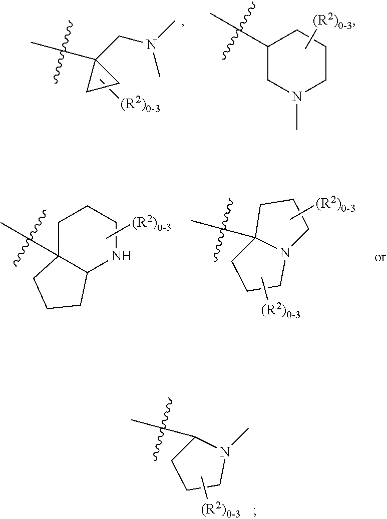

X is a bond, N, methylene, ethylene or propylene;
D is CO or SO₂;
E is N or CR¹⁰;
Y is a bond, O or NR⁵;
Z is hydrogen, C1-C4 alkyl, heterocyclyl, heteroaryl or aryl, where Z is optionally substituted with 1-3 substituents selected from halogen, C1-C4 alkyl, C1-C4 haloalkyl, oxo, OR¹², N(R¹²)₂, COR¹² and cyano;
each R² is independently hydrogen, hydroxy, halogen, C1-C3 alkyl, spiro cyclopropyl, C1-C3 cyanoalkyl, C1-C3 hydroxyalkyl, CHF₂, HC(═O)—, —OC(O)N(R⁵)₂, —CO₂R⁵, —CO₂N(R⁵)₂, ═CH₂, ═CHR¹¹ or ═C(R¹¹)₂, wherein R² is optionally substituted with one or more R⁴;
R³ is aryl or heteroaryl, wherein the aryl or the heteroaryl is optionally substituted with one or more R⁸;
R⁴ is hydrogen, halogen or C1-C3 alkyl;
each R⁵ is independently hydrogen or C1-C3 alkyl;
each R⁶ is independently hydrogen, hydroxy, C1-C4 alkyl, C1-C4 hydroxyalkyl or heteroaryl, or two R⁶ join to form C3-C6 cycloalkyl or heterocycle;
each R⁸ is independently halogen, cyano, hydroxy, cycloalkyl, C1-C3 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, or —S—C1-C3 haloalkyl;
each R⁹ is independently hydrogen, C1-C4 alkyl, methoxy, CH₂-methoxy, hydroxy, CH₂F, CF₂, CF₃, C—CN, cyclopropyl, or two R⁹ on the same or different carbon atoms join to form a C1-C2 alkylene bridge or a spirocycle;
each R¹⁰ is hydrogen, halogen, CH₂F, CHF₂, CF₃, C1-C3 alkyl, cyclopropyl, OR¹², or N(R¹²)₂;
each R¹¹ is halogen or C1-C3 alkyl,
R¹² is C1-C3 alkyl, and
R¹³ is H or forms a ring with A.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting KRas G12S and/or KRas G12C activity in a in a cell, comprising contacting the cell with a compound of Formula (Ia) or (Ib), or a pharmaceutically acceptable salt thereof, or a pharmaceutical composition thereof as defined herein. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula (Ia) or (Ib), or a pharmaceutically acceptable salt thereof, or a pharmaceutical composition thereof as defined herein.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method of treating a KRas G12S-associated and/or KRas G12C-associated disease or disorder in a patient in need of such treatment, the method comprising administering to the patient a therapeutically effective amount of a compound of Formula (Ia) or (Ib), or a pharmaceutically acceptable salt thereof, or a pharmaceutical composition thereof as defined herein.

Also provided herein is a compound of Formula (Ia) or (Ib), or a pharmaceutically acceptable salt thereof, or a pharmaceutical composition thereof as defined herein for use in therapy.

Also provided herein is a compound of Formula (Ia) or (Ib), or a pharmaceutically acceptable salt thereof or a pharmaceutical composition thereof as defined herein for use in the treatment of cancer.

Also provided herein is a compound of Formula (Ia) or (Ib), or a pharmaceutically acceptable salt thereof for use in the inhibition of KRas G12S and/or KRas G12C.

Also provided herein is a compound of Formula (Ia) or (Ib), or a pharmaceutically acceptable salt thereof or a pharmaceutical composition thereof as defined herein, for use in the treatment of a KRas G12S-associated and/or KRas G12C-associated disease or disorder.

Also provided herein is the use of a compound of Formula (Ia) or (Ib), or a pharmaceutically acceptable salt thereof, as defined herein in the manufacture of a medicament for the treatment of cancer.

Also provided herein is a use of a compound of Formula (Ia) or (Ib), or a pharmaceutically acceptable salt thereof, as defined herein in the manufacture of a medicament for the inhibition of activity of KRas G12S and/or KRas G12C.

Also provided herein is the use of a compound of Formula (Ia) or (Ib), or a pharmaceutically acceptable salt thereof, as defined herein, in the manufacture of a medicament for the treatment of a KRas G12S-associated and/or KRas G12C-associated disease or disorder.

Also provided herein is a method for treating cancer in a patient in need thereof, the method comprising (a) determining that the cancer is associated with a KRas G12S mutation (i.e., a KRas G12S-associated cancer) or a KRas G12C mutation (i.e., a KRas G12C-associated cancer); and (b) administering to the patient a therapeutically effective amount of a compound of Formula (Ia) or (Ib), or a pharmaceutically acceptable salt thereof, or a pharmaceutical composition thereof.

Also provided herein is a process for preparing a compound of Formula (Ia) or (Ib), or a pharmaceutically acceptable salt thereof.

Also provided herein is a compound of Formula (Ia) or (Ib), or a pharmaceutically acceptable salt thereof obtained by a process of preparing the compound as defined herein.

### DETAILED DESCRIPTION OF THE INVENTION

The present invention relates to inhibitors of KRas G12S and/or KRas G12C. In particular, the present invention relates to compounds that inhibit the activity of KRas G12S and/or KRas G12C, pharmaceutical compositions comprising a therapeutically effective amount of the compounds and methods of use therefor.

### DEFINITIONS

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12S” refers to a mutant form of a mammalian KRas protein that contains an amino acid substitution of a serine for a glycine at amino acid position 12. The assignment of amino acid codon and residue positions for human KRas is based on the amino acid sequence identified by UniProtKB/Swiss-Prot P01116: Variantp.Glyl2Asp.

As used herein, a “KRas G12S inhibitor” refers to compounds of the present invention that are represented by Formula (Ia) or (Ib), as described herein. These compounds are capable of negatively modulating or inhibiting all or a portion of the enzymatic activity of KRas G12S.

A “KRas G12S-associated disease or disorder” as used herein refers to diseases or disorders associated with or mediated by or having a KRas G12S mutation. A non-limiting example of a KRas G12S-associated disease or disorder is a KRas G12S-associated cancer.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly 12Cys.

As used herein, a “KRas G12C inhibitor” refers to compounds of the present invention that are represented by Formula (Ia) or (Ib), as described herein. These compounds are capable of negatively modulating or inhibiting all or a portion of the enzymatic activity of KRas G12C.

A “KRas G12C-associated disease or disorder” as used herein refers to diseases or disorders associated with or mediated by or having a KRas G12C mutation. A non-limiting example of a KRas G12C-associated disease or disorder is a KRas G12C-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12S or a KRas G12C mutation (e.g., as determined using a regulatory agency-approved, e.g., FDA-approved, assay or kit). In some embodiments, the subject has a tumor that is positive for a KRas G12S or a KRas G12C mutation (e.g., as determined using a regulatory agency-approved assay or kit). The subject can be a subject with a tumor(s) that is positive for a KRas G12S or a KRas G12C mutation (e.g., identified as positive using a regulatory agency-approved, e.g., FDA-approved, assay or kit). The subject can be a subject whose tumors have a KRas G12S or a KRas G12C mutation (e.g., where the tumor is identified as such using a regulatory agency-approved, e.g., FDA-approved, kit or assay). In some embodiments, the subject is suspected of having a KRas G12S gene-associated cancer or a KRas G12C gene-associated cancer. In some embodiments, the subject has a clinical record indicating that the subject has a tumor that has a KRas G12S or a KRas G12C mutation (and optionally the clinical record indicates that the subject should be treated with any of the compositions provided herein).

In some embodiments of any of the methods or uses described herein, an assay is used to determine whether the patient has KRas G12S or KRas G12C mutation using a sample (e.g., a biological sample or a biopsy sample (e.g., a paraffin-embedded biopsy sample) from a patient (e.g., a patient suspected of having a KRas G12S-associated or a KRas G12C-associated cancer, a patient having one or more symptoms of a KRas G12S-associated or a KRas G12C-associated cancer, and/or a patient that has an increased risk of developing a KRas G12S-associated cancer or a KRas G12C-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “acyl” refers to —C(O)CH₃.

The terms “C1-C6 alkyl”, “C1-C4 alkyl” and “C1-C3 alkyl” as employed herein refers to straight and branched chain aliphatic groups having from 1-6 carbon atoms, or 1-4 carbon atoms, or 1-3 carbon atoms, respectively. Examples of alkyl groups include, without limitation, methyl, ethyl, propyl, isopropyl, butyl, isobutyl, sec-butyl, tert-butyl, pentyl, and hexyl.

The terms “C1-C3 haloalkyl” and “C1-C4 haloalkyl” refer to a C1-C3 alkyl chain or C1-C4 alkyl chain, respectively, as defined herein in which one or more hydrogen has been replaced by a halogen. Examples include trifluoromethyl, difluoromethyl and fluoromethyl.

An “C1-C4 alkylene,” group is a C1-C4 alkyl group, as defined hereinabove, that is positioned between and serves to connect two other chemical groups. Exemplary alkylene groups include, without limitation, methylene, ethylene, 2-2-dimethyl ethylene, propylene, and butylene.

The terms “C1-C3 alkoxy” and “C1-C4 alkoxy” refer to —OC1-C3 alkyl and —OC1-C4 alkyl, respectively, wherein the alkyl portion is as defined herein above.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted with one or more R^{X} groups as defined herein. The cycloalkyl may be a monocyclic, a bicyclic, a spirocyclic or a bridged ring system. Examples of cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkyl” also includes bridged cycloalkyls, such as bicyclo[1.1.1]pentanyl.

As used herein, the terms “C1-C3 hydroxyalkyl” and “C1-C4 hydroxyalkyl” refer to —C1-C3 alkylene-OH and —C1-C4 alkylene-OH, respectively.

As used herein, the term “C2-C4 hydroxyalkynyl” refers to —C2-C4 alkynylene-OH.

An “aryl” group is a C6-C14 aromatic moiety comprising one to three aromatic rings, which is optionally substituted with one or more substituents as defined herein. As one embodiment, the aryl group is a C6-C10 aryl group. Examples of aryl groups include, without limitation, phenyl, naphthyl, anthracenyl, fluorenyl, and dihydrobenzofuranyl. “Aryl” also refers to bicyclic or tricyclic ring systems in which one or two rings, respectively, of said aryl ring system may be saturated or partially saturated, and wherein if said ring system includes two saturated rings, said saturated rings may be fused or spirocyclic. An example of an aryl ring system comprising two saturated rings wherein the rings are spirocyclic includes the following ring system:

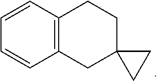 [SMILES: c1ccc2c(c1)CCC1(CC1)C2]

An “araC1-C6 alkyl” or “arylalkyl” group comprises an aryl group covalently linked to an alkyl group, either of which may independently be optionally substituted or unsubstituted. An example of an aralkyl group is (C6-C10)aryl(C1-C6)alkyl-, including, without limitation, benzyl, phenethyl, and naphthylmethyl. Another example of an aralkyl group is (C6-C10)aryl(C1-C3)alkyl-, again including, without limitation, benzyl, phenethyl, and naphthylmethyl. An example of a substituted araC1-C6 alkyl is wherein the alkyl group is substituted with hydroxyalkyl.

A “heterocyclyl” or “heterocyclic” group is a ring structure having from 3 to 12 atoms, for example 4 to 8 atoms, wherein one or more atoms are selected from the group consisting of N, O, and S wherein the ring N atom may be oxidized to N—O, and the ring S atom may be oxidized to SO or SO₂, the remainder of the ring atoms being carbon. The heterocyclyl may be a monocyclic, a bicyclic, a spirocyclic or a bridged ring system. The heterocyclic group is optionally substituted with one or more Re on ring carbon or ring nitrogen at one or more positions, wherein R⁶ is as defined for Formula I. The heterocyclic group is also independently optionally substituted on a ring nitrogen atom with alkyl, aralkyl, alkylcarbonyl, or on sulfur with lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, thiomorpholinyl 1,1 dioxide, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, tetrahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 6,7-dihydro-5H-pyrrolo[1,2-a]imidazole, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. “Heteroaryl” also refers to bicyclic ring systems having, in addition to carbon atoms, from one to three heteroatoms per ring selected from the group consisting of N, O, and S in which one ring system may be saturated or partially saturated.

As used herein, “an effective amount” of a compound is an amount that is sufficient to negatively modulate or inhibit the activity of KRas G12S and/or KRas G12C. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of KRas G12S and/or KRas G12C. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

### Compounds

In one aspect of the invention, compounds are provided represented by Formula (Ia) or (Ib):

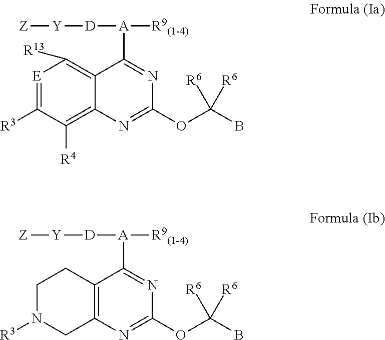

or a pharmaceutically acceptable salt thereof, wherein:

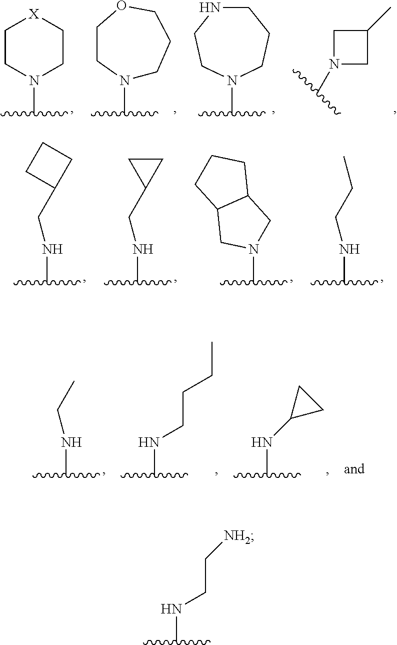

A is selected from:

B is selected from:

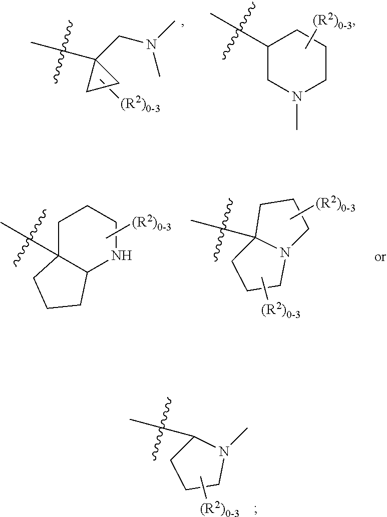

X is a bond, N, methylene, ethylene or propylene;
D is CO or SO₂;
E is N or CR¹⁰;
Y is a bond, O or NR⁵;
Z is hydrogen, C1-C4 alkyl, heterocyclyl, heteroaryl or aryl, where Z is optionally substituted with 1-3 substituents selected from halogen, C1-C4 alkyl, C1-C4 haloalkyl, oxo, OR¹², N(R¹²)₂, COR¹² and cyano;
each R² is independently hydrogen, hydroxy, halogen, C1-C3 alkyl, spiro cyclopropyl, C1-C3 cyanoalkyl, C1-C3 hydroxyalkyl, CHF₂, HC(═O)—, —OC(O)N(R⁵)₂, —CO₂R⁵, —CO₂N(R⁵)₂, —CH₂, ═CHR¹¹ or ═C(R¹¹)₂, wherein R² is optionally substituted with one or more R⁴;
R³ is aryl or heteroaryl, wherein the aryl or the heteroaryl is optionally substituted with one or more R⁸;
R⁴ is hydrogen, halogen or C1-C3 alkyl;
each R⁵ is independently hydrogen or C1-C3 alkyl;
each R⁶ is independently hydrogen, hydroxy, C1-C4 alkyl, C1-C4 hydroxyalkyl or heteroaryl, or two R⁶ join to form C3-C6 cycloalkyl or heterocycle;
each R⁸ is independently halogen, cyano, hydroxy, cycloalkyl, C1-C3 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, or —S—C1-C3 haloalkyl;
each R⁹ is independently hydrogen, C1-C4 alkyl, methoxy, CH₂-methoxy, hydroxy, CH₂F, CF₂, CF₃, C—CN, cyclopropyl, or two R⁹ on the same or different carbon atoms join to form a C1-C2 alkylene bridge or a spirocycle;
each R¹⁰ is hydrogen, halogen, CH₂F, CHF₂, CF₃, C1-C3 alkyl, cyclopropyl, OR¹², or N(R¹²)₂;
each R¹¹ is halogen or C1-C3 alkyl,
R¹² is C1-C3 alkyl, and
R¹³ is H or forms a ring with A.

In certain such embodiments, Formula (Ia) is Formula IA:

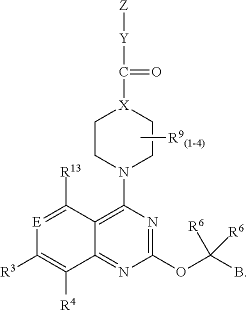

In other such embodiments, Formula (Ia) is Formula IB:

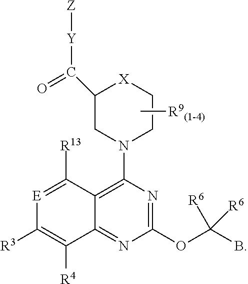

In certain embodiments, X is methylene.

In certain embodiments, X is ethylene.

In certain embodiments, X is a bond.

In certain embodiments, X is propylene.

In one embodiment, Y is NR⁵.

In one embodiment, Y is a bond.

In one embodiment, Y is O.

In one embodiment, Z is hydrogen.

In another embodiment, Z is C1-C4 alkyl, optionally substituted with 1-3 halogens.

In yet another embodiment, Z is aryl, optionally substituted with 1-3 halogens, cyano, or C1-C4 haloalkyl. In an aspect of this embodiment, the aryl is phenyl.

In yet another embodiment, Z is heteroaryl, optionally substituted with 1-2 C1-C4 alkyl. In an aspect of this embodiment, the heteroaryl is pyrazolyl or pyridinyl.

In yet another embodiment of the invention, Z is heterocyclyl, optionally substituted with oxo. In an aspect of this embodiment, the heterocyclyl is oxazolidine.

On one embodiment, two R⁹ join to form a methylene bridge, or two R⁹ join to form an ethylene bridge.

In certain embodiments, R⁵ is hydrogen.

In certain embodiments, R⁵ is C1-C3 alkyl. In certain of these embodiments, the C1-C3 alkyl is methyl.

In one embodiment, R² is halogen. In an aspect of this embodiment R² is fluoro.

In another embodiment, R² is ═CHR¹¹.

In yet another embodiment, R² is ═C(R¹¹)₂.

In one embodiment of the compounds of Formula (Ia) or (Ib), Formula (IA) and/or Formula (IB), R³ is aryl optionally substituted with one or more R⁸. In certain embodiments, the aryl is selected from the group consisting of phenyl, naphthyl, 1,2,3,4-tetrahydronaphthalenyl and 2,3-dihydro-1H-indenyl, wherein each is optionally substituted with one or more R⁸.

In one embodiment, the aryl is phenyl substituted with one or more R⁸ groups. In one embodiment, the aryl is phenyl substituted with one or more R⁸ groups independently selected from halogen, C1-C3 alkyl and cycloalkyl. In certain embodiments the phenyl is substituted with two R⁸ groups. In certain embodiments the phenyl is substituted with two R⁸ groups, wherein the one R⁸ group is halogen and the other R⁸ group is C1-C3 alkyl or cycloalkyl.

In one embodiment, the aryl is 2,3-dihydro-1H-indenyl optionally substituted with one or more R⁸. In one embodiment, the aryl is 2,3-dihydro-1H-indenyl optionally substituted with one R⁸. In one embodiment, R⁸ is C1-C alkyl.

In one embodiment, the aryl is naphthyl substituted with one or more R⁸ groups. In one embodiment, the aryl is naphthyl substituted with one or more R⁸ groups independently selected from halogen, cyano, hydroxy, C1-C3 alkyl, —S—C1-C3 alkyl, C2-C4 alkenyl, C2-C4 alkynyl, C2-C4 hydroxyalkynyl, C1-C3 cyanoalkyl, cycloalkyl, triazolyl, C1-C3 haloalkyl and —O—C1-C3 haloalkyl.

In one embodiment, the aryl is naphthyl substituted with hydroxy. In one embodiment, the aryl is naphthyl substituted with halogen. In certain embodiments, the halogen is chlorine, fluorine or bromine. In other embodiments, the halogen is chlorine. In other embodiments, the halogen is fluorine.

In one embodiment, the aryl is naphthyl substituted with C1-C3 alkyl, wherein the C1-C3 alkyl is methyl or ethyl.

In one embodiment, the aryl is naphthyl substituted with C2-C4 alkenyl. In certain embodiments, the C2-C4 alkenyl is prop-2-enyl.

In one embodiment, the aryl is naphthyl substituted with C2-C4 alkynyl. In certain embodiments, the C2-C4 alkynyl is ethyne or prop-2-ynyl.

In one embodiment, the aryl is naphthyl substituted with cycloalkyl. In certain embodiments, the cycloalkyl is cyclopropyl.

In one embodiment, the aryl is naphthyl substituted with one or two R⁸, wherein each R⁸ is halogen, cyano, hydroxy, C1-C3 alkyl, —S—C1-C3 alkyl, C2-C4 alkenyl, C2-C4 alkynyl, C2-C4 hydroxyalkynyl, C1-C3 cyanoalkyl, or triazolyl. In one embodiment, the aryl is naphthyl substituted with two R⁸ groups independently selected from halogen, hydroxy, C1-C3 alkyl and C2-C4 alkynyl. In another embodiment, one R⁸ is hydroxy and the other R⁸ is C1-C3 alkyl, halogen or C2-C4 alkynyl, or one R⁸ is halogen and the other R⁸ is C1-C3 or C2-C4 alkynyl

In one embodiment, the aryl is naphthyl substituted with three R⁸ groups wherein the first R⁸ group is halogen, the second R⁸ group is hydroxy, and the third R⁸ group is C1-C3 alkyl or C2-C4 alkynyl.

In one embodiment of the compounds of Formula (Ia) or (Ib), Formula (IA) and/or Formula (IB), R³ is heteroaryl optionally substituted with one or more R⁸. In one embodiment, the heteroaryl is isoquinolinyl, indazolyl, or benzo[d][1,3]dioxolyl optionally substituted with one or more R⁸. In one embodiment, the heteroaryl is indazolyl optionally substituted with one or more R⁸. In one embodiment, the heteroaryl is indazolyl optionally substituted with C1-C3 alkyl or C1-C3 alkyl and halogen. In other embodiments, the heteroaryl is isoquinolinyl optionally substituted with one or more R⁸. In other embodiments, the heteroaryl is isoquinolinyl optionally substituted with halogen or C2-C4 alkynyl. In certain embodiments, the heteroaryl is benzo[d][1,3]dioxolyl optionally substituted with two R⁸ groups. In certain embodiments, the heteroaryl is benzo[d][1,3]dioxolyl optionally substituted with two R⁸ groups, wherein each R⁸ group is an independently selected halogen. In one embodiment, the two halogens are gem-difluoro substitutions. In another embodiment R³ is indazolyl substituted with two R⁸, where one R⁸ is halogen and the other R⁸ is C1-C3 alkyl.

In one embodiment of the compounds of Formula (Ia) or (Ib), Formula (IA) and/or Formula (IB), R⁴ is hydrogen.

In one embodiment of the compounds of Formula (Ia) or (Ib), Formula (IA) and/or Formula (IB), R⁴ is halogen. In one embodiment, R⁴ is fluorine. In one embodiment, R⁴ is chlorine.

In one embodiment of the compounds of Formula (Ia) or (Ib), Formula (IA) and/or Formula (IB), R⁴ is C1-C3 alkyl. In one embodiment, R⁴ is methyl.

Nonlimiting examples of compounds of Formula (Ia) or (Ib), Formula (IA) and/or Formula (IB) are selected from the group consisting of:

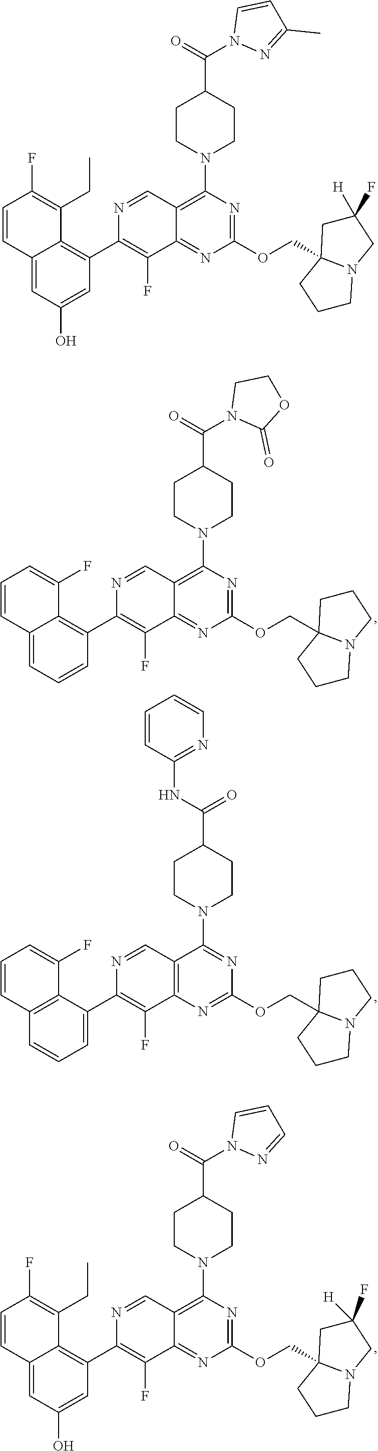

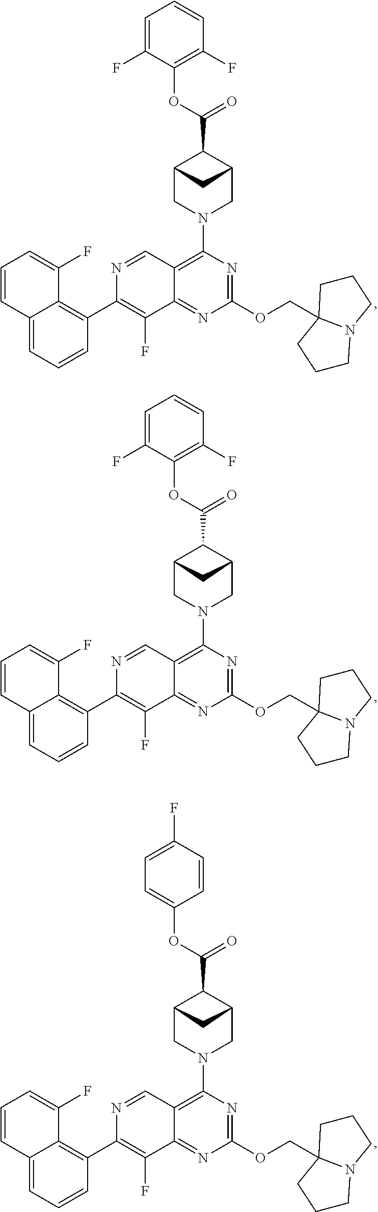 [SMILES: O=C(Oc1c(F)cccc1F)[C@@H]1[C@@H]2C[C@H]1CN(c1nc(OCC34CCCN3CCC4)nc3c(F)c(-c4cccc5cccc(F)c45)ncc13)C2 ; O=C(Oc1c(F)cccc1F)[C@H]1[C@@H]2C[C@H]1CN(c1nc(OCC34CCCN3CCC4)nc3c(F)c(-c4cccc5cccc(F)c45)ncc13)C2 ; O=C(Oc1ccc(F)cc1)[C@@H]1[C@@H]2C[C@H]1CN(c1nc(OCC34CCCN3CCC4)nc3c(F)c(-c4cccc5cccc(F)c45)ncc13)C2]

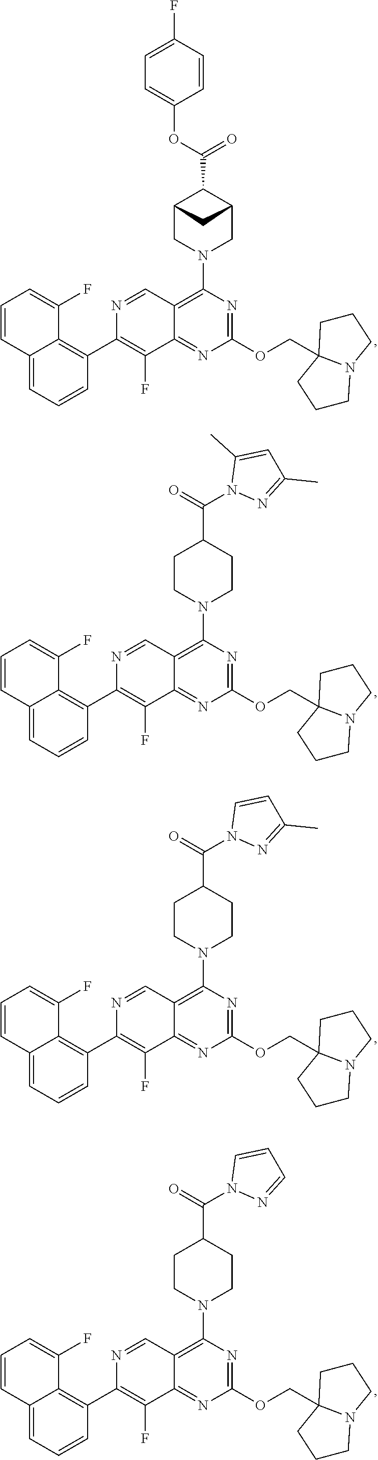 [SMILES: O=C(Oc1ccc(F)cc1)[C@H]1[C@@H]2C[C@H]1CN(c1nc(OCC34CCCN3CCC4)nc3c(F)c(-c4cccc5cccc(F)c45)ncc13)C2 ; Cc1cc(C)n(C(=O)C2CCN(c3nc(OCC45CCCN4CCC5)nc4c(F)c(-c5cccc6cccc(F)c56)ncc34)CC2)n1 ; Cc1ccn(C(=O)C2CCN(c3nc(OCC45CCCN4CCC5)nc4c(F)c(-c5cccc6cccc(F)c56)ncc34)CC2)n1 ; O=C(C1CCN(c2nc(OCC34CCCN3CCC4)nc3c(F)c(-c4cccc5cccc(F)c45)ncc23)CC1)n1cccn1]

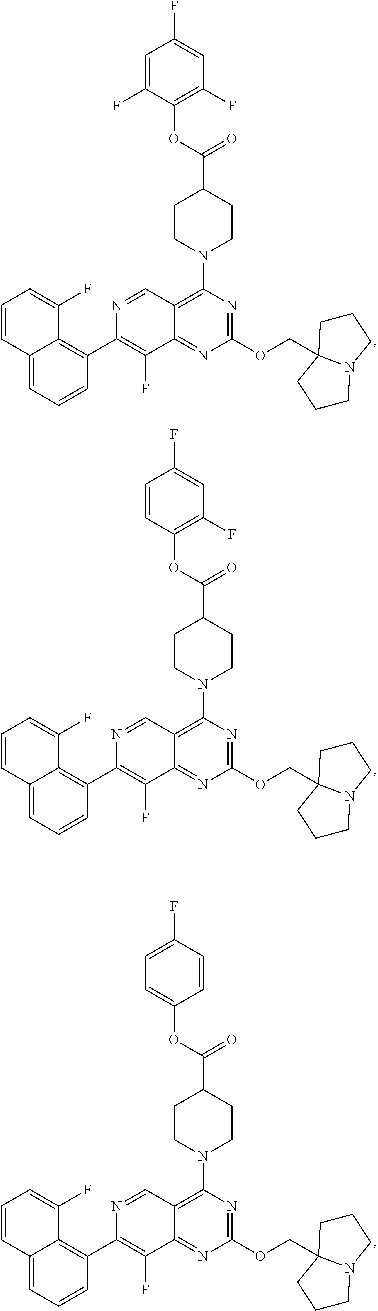 [SMILES: O=C(Oc1c(F)cc(F)cc1F)C1CCN(c2nc(OCC34CCCN3CCC4)nc3c(F)c(-c4cccc5cccc(F)c45)ncc23)CC1 ; O=C(Oc1ccc(F)cc1F)C1CCN(c2nc(OCC34CCCN3CCC4)nc3c(F)c(-c4cccc5cccc(F)c45)ncc23)CC1 ; O=C(Oc1ccc(F)cc1)C1CCN(c2nc(OCC34CCCN3CCC4)nc3c(F)c(-c4cccc5cccc(F)c45)ncc23)CC1]

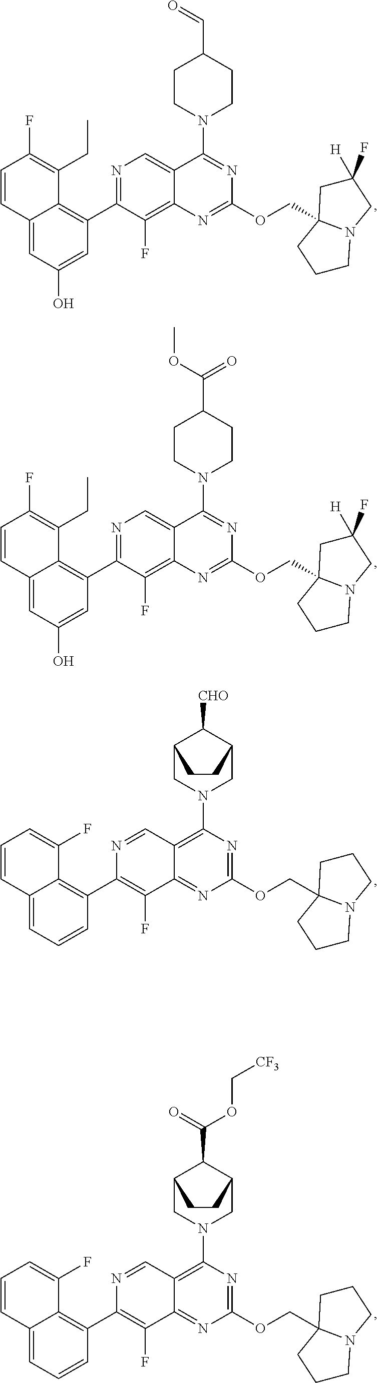 [SMILES: CCc1c(F)ccc2cc(O)cc(-c3ncc4c(N5CCC(C=O)CC5)nc(OC[C@@]56CCCN5C[C@H](F)C6)nc4c3F)c12 ; CCc1c(F)ccc2cc(O)cc(-c3ncc4c(N5CCC(C(=O)OC)CC5)nc(OC[C@@]56CCCN5C[C@H](F)C6)nc4c3F)c12 ; O=C[C@@H]1[C@@H]2CC[C@H]1CN(c1nc(OCC34CCCN3CCC4)nc3c(F)c(-c4cccc5cccc(F)c45)ncc13)C2 ; O=C(OCC(F)(F)F)[C@@H]1[C@@H]2CC[C@H]1CN(c1nc(OCC34CCCN3CCC4)nc3c(F)c(-c4cccc5cccc(F)c45)ncc13)C2]

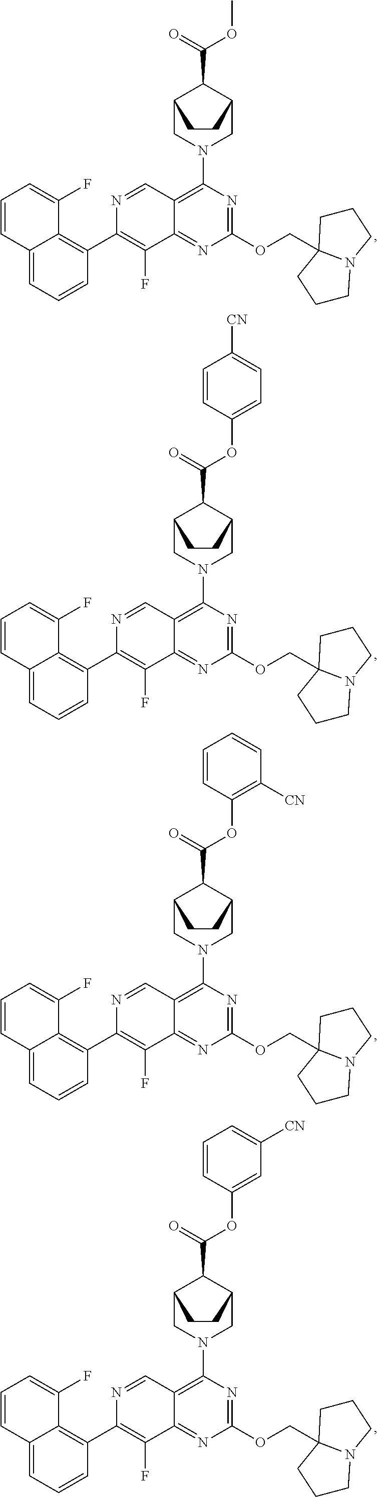

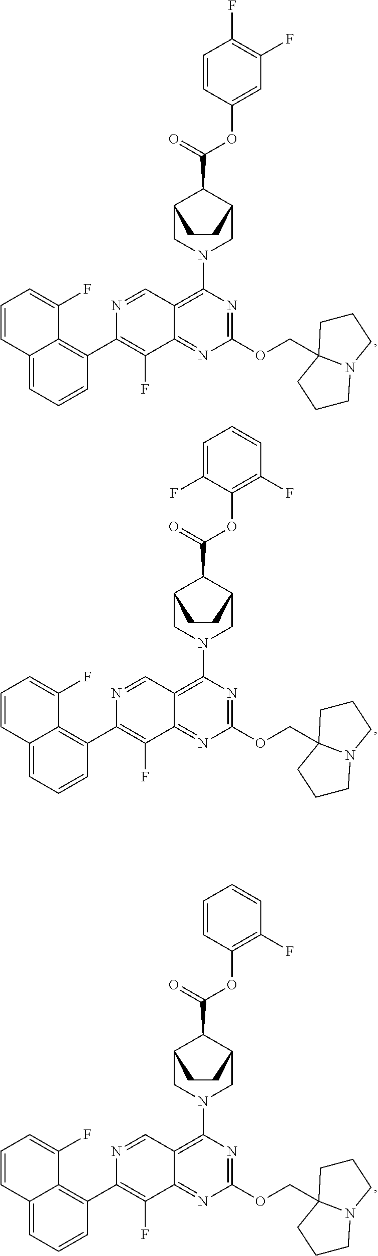 [SMILES: O=C(Oc1ccc(F)c(F)c1)[C@@H]1[C@@H]2CC[C@H]1CN(c1nc(OCC34CCCN3CCC4)nc3c(F)c(-c4cccc5cccc(F)c45)ncc13)C2 ; O=C(Oc1c(F)cccc1F)[C@@H]1[C@@H]2CC[C@H]1CN(c1nc(OCC34CCCN3CCC4)nc3c(F)c(-c4cccc5cccc(F)c45)ncc13)C2 ; O=C(Oc1ccccc1F)[C@@H]1[C@@H]2CC[C@H]1CN(c1nc(OCC34CCCN3CCC4)nc3c(F)c(-c4cccc5cccc(F)c45)ncc13)C2]

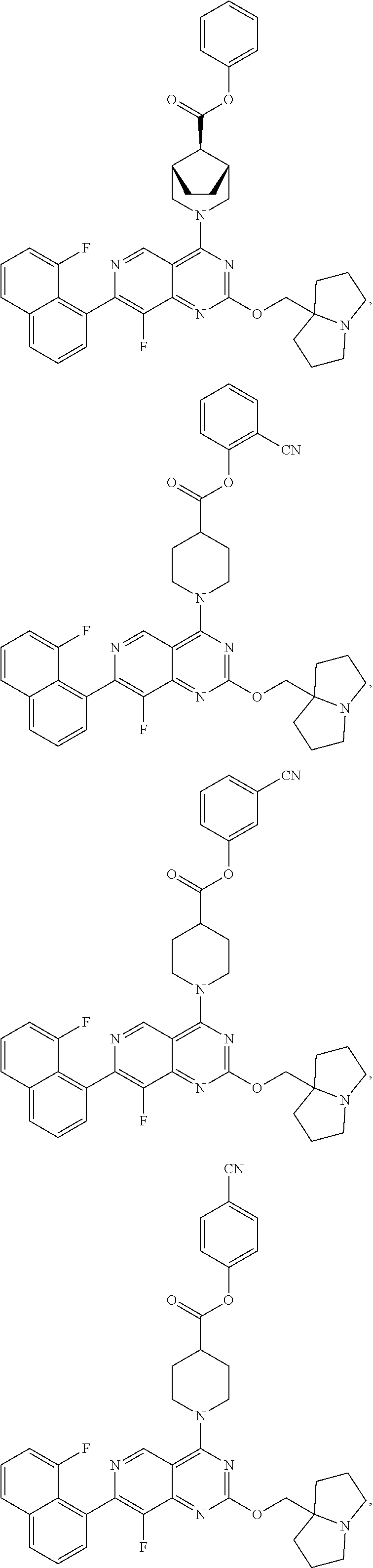

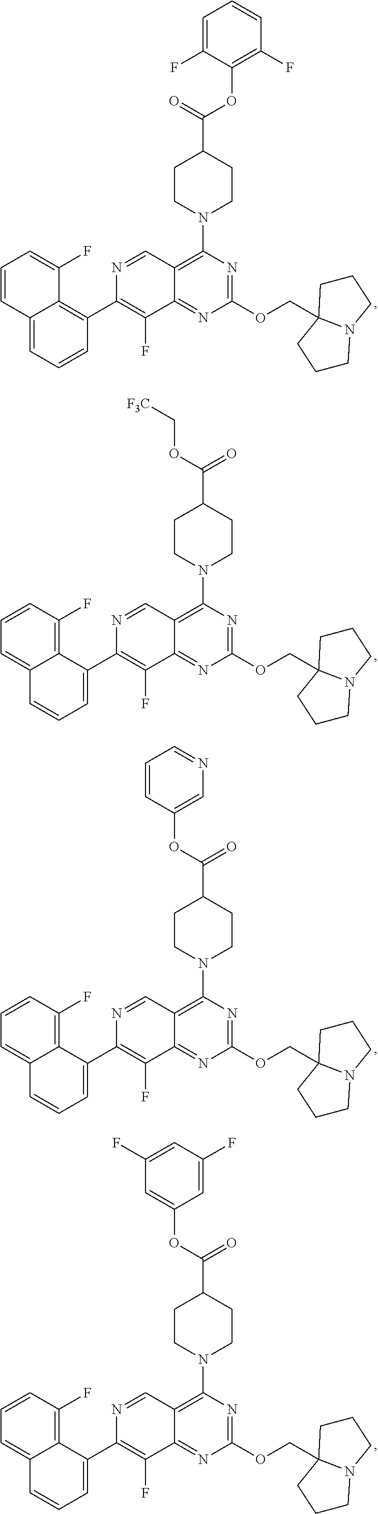 [SMILES: O=C(Oc1c(F)cccc1F)C1CCN(c2nc(OCC34CCCN3CCC4)nc3c(F)c(-c4cccc5cccc(F)c45)ncc23)CC1 ; O=C(OCC(F)(F)F)C1CCN(c2nc(OCC34CCCN3CCC4)nc3c(F)c(-c4cccc5cccc(F)c45)ncc23)CC1 ; O=C(Oc1cccnc1)C1CCN(c2nc(OCC34CCCN3CCC4)nc3c(F)c(-c4cccc5cccc(F)c45)ncc23)CC1 ; O=C(Oc1cc(F)cc(F)c1)C1CCN(c2nc(OCC34CCCN3CCC4)nc3c(F)c(-c4cccc5cccc(F)c45)ncc23)CC1]

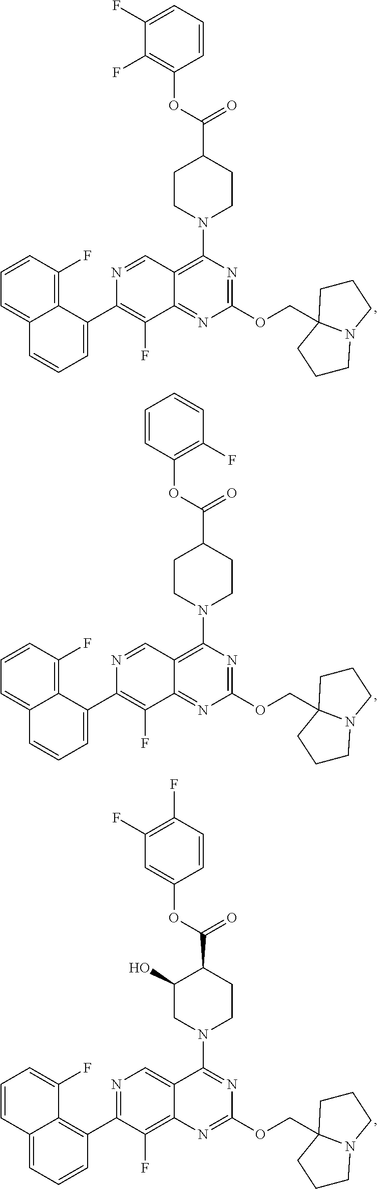 [SMILES: O=C(Oc1cccc(F)c1F)C1CCN(c2nc(OCC34CCCN3CCC4)nc3c(F)c(-c4cccc5cccc(F)c45)ncc23)CC1 ; O=C(Oc1ccccc1F)C1CCN(c2nc(OCC34CCCN3CCC4)nc3c(F)c(-c4cccc5cccc(F)c45)ncc23)CC1 ; O=C(Oc1ccc(F)c(F)c1)[C@H]1CCN(c2nc(OCC34CCCN3CCC4)nc3c(F)c(-c4cccc5cccc(F)c45)ncc23)C[C@H]1O]

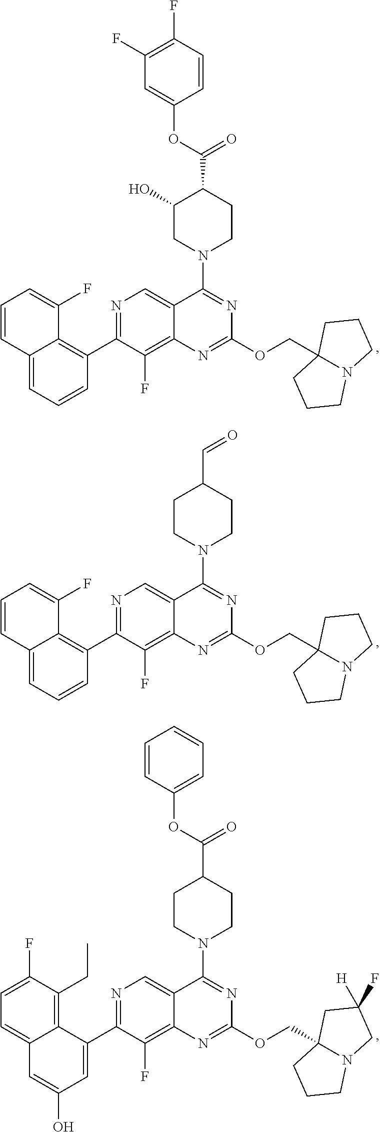 [SMILES: O=C(Oc1ccc(F)c(F)c1)[C@@H]1CCN(c2nc(OCC34CCCN3CCC4)nc3c(F)c(-c4cccc5cccc(F)c45)ncc23)C[C@@H]1O ; O=CC1CCN(c2nc(OCC34CCCN3CCC4)nc3c(F)c(-c4cccc5cccc(F)c45)ncc23)CC1 ; CCc1c(F)ccc2cc(O)cc(-c3ncc4c(N5CCC(C(=O)Oc6ccccc6)CC5)nc(OC[C@@]56CCCN5C[C@H](F)C6)nc4c3F)c12]

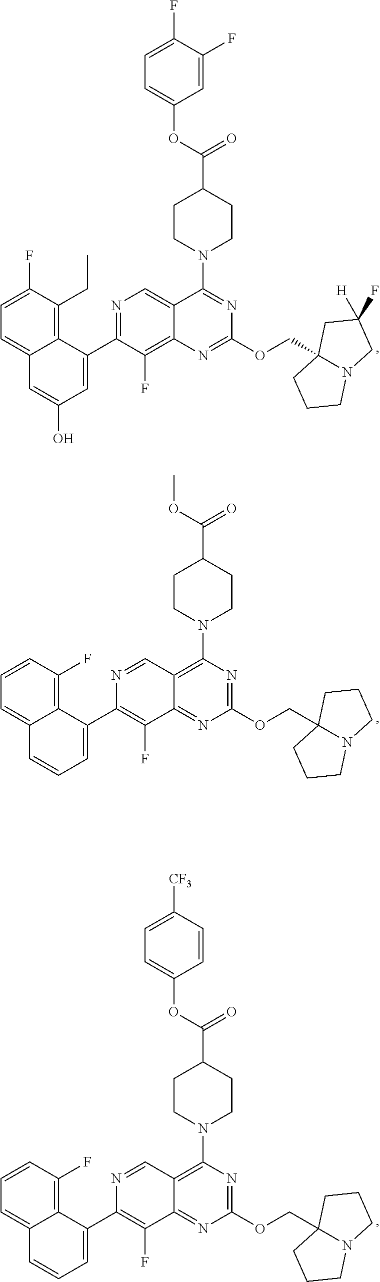 [SMILES: CCc1c(F)ccc2cc(O)cc(-c3ncc4c(N5CCC(C(=O)Oc6ccc(F)c(F)c6)CC5)nc(OC[C@@]56CCCN5C[C@H](F)C6)nc4c3F)c12 ; COC(=O)C1CCN(c2nc(OCC34CCCN3CCC4)nc3c(F)c(-c4cccc5cccc(F)c45)ncc23)CC1 ; O=C(Oc1ccc(C(F)(F)F)cc1)C1CCN(c2nc(OCC34CCCN3CCC4)nc3c(F)c(-c4cccc5cccc(F)c45)ncc23)CC1]

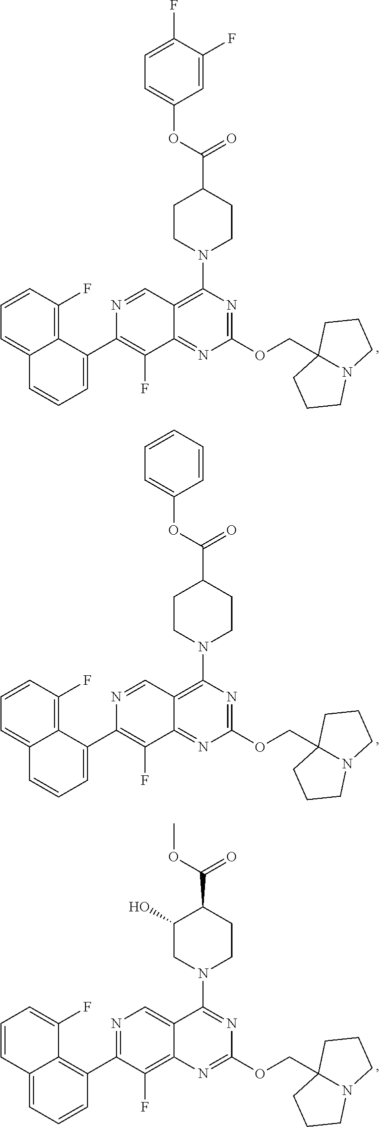 [SMILES: O=C(Oc1ccc(F)c(F)c1)C1CCN(c2nc(OCC34CCCN3CCC4)nc3c(F)c(-c4cccc5cccc(F)c45)ncc23)CC1 ; O=C(Oc1ccccc1)C1CCN(c2nc(OCC34CCCN3CCC4)nc3c(F)c(-c4cccc5cccc(F)c45)ncc23)CC1 ; COC(=O)[C@H]1CCN(c2nc(OCC34CCCN3CCC4)nc3c(F)c(-c4cccc5cccc(F)c45)ncc23)C[C@@H]1O]

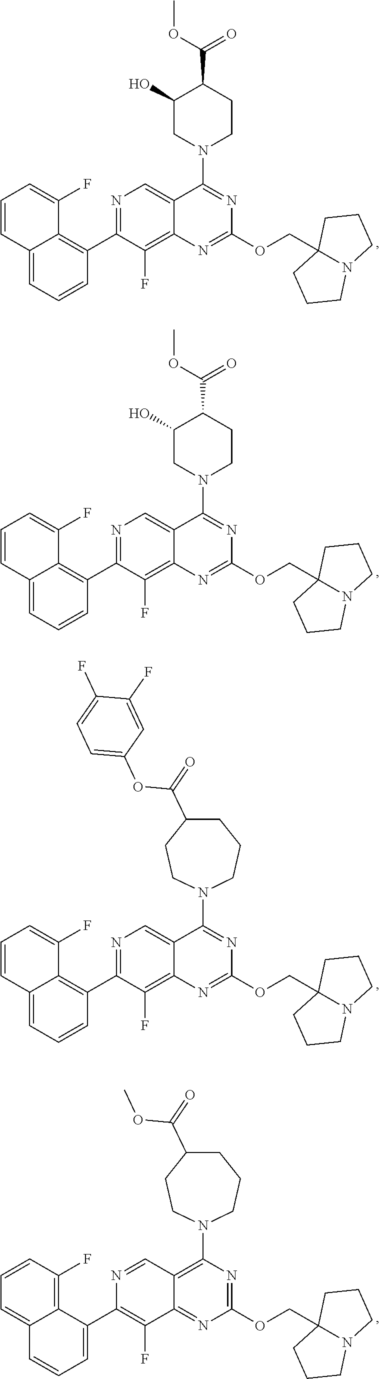

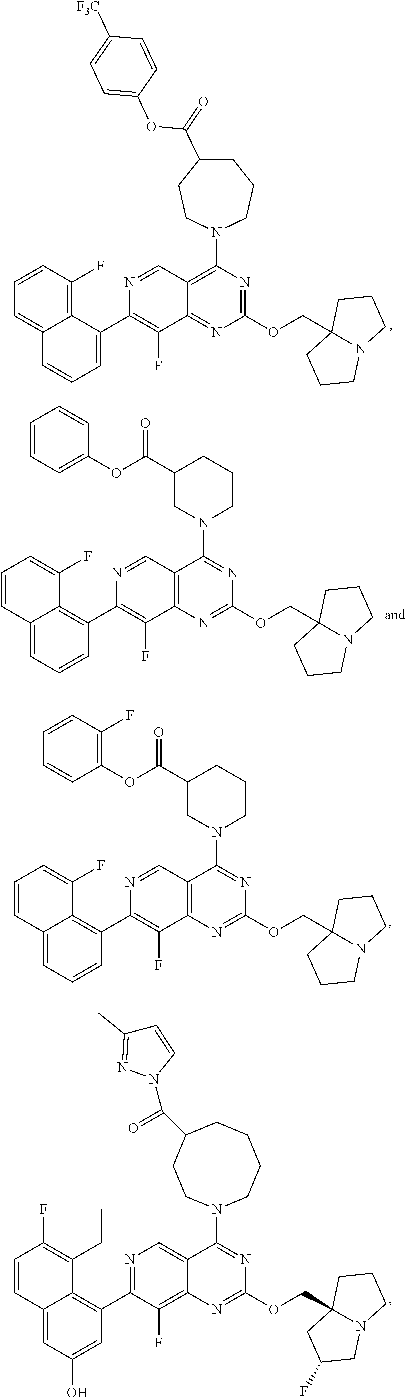

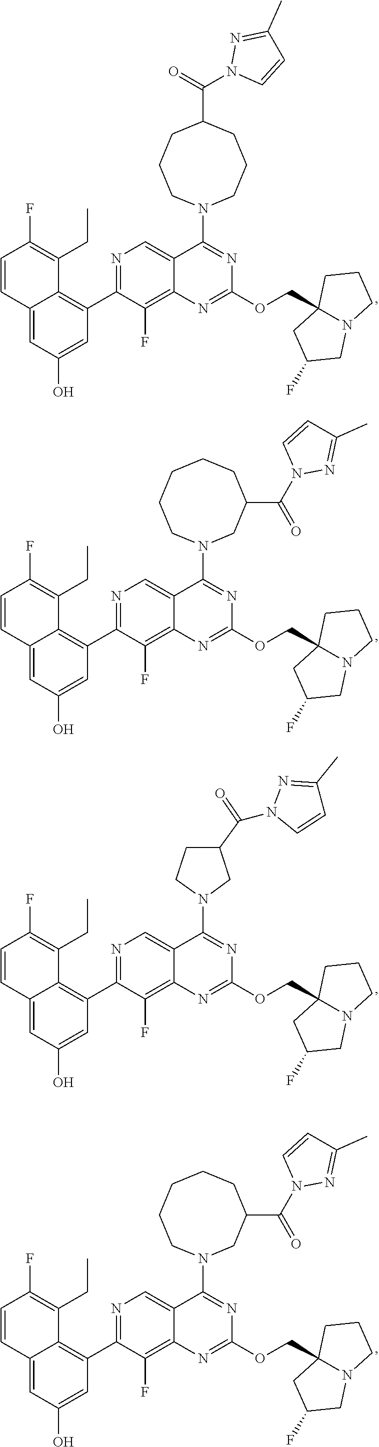

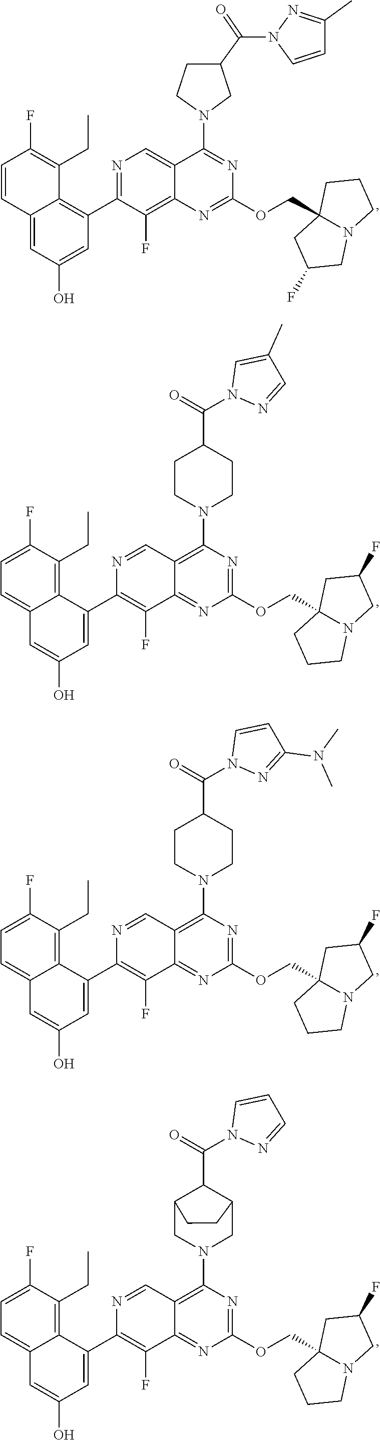

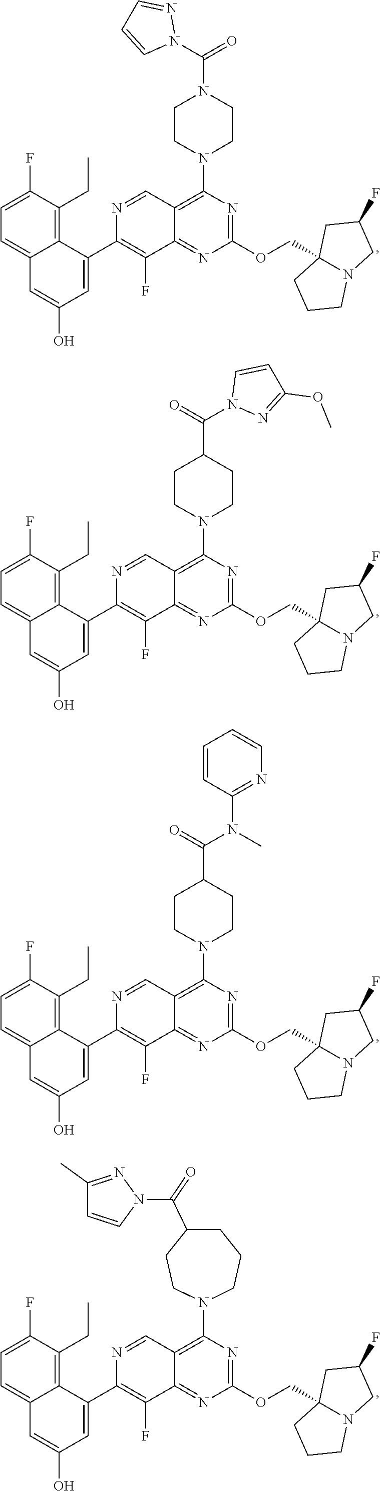

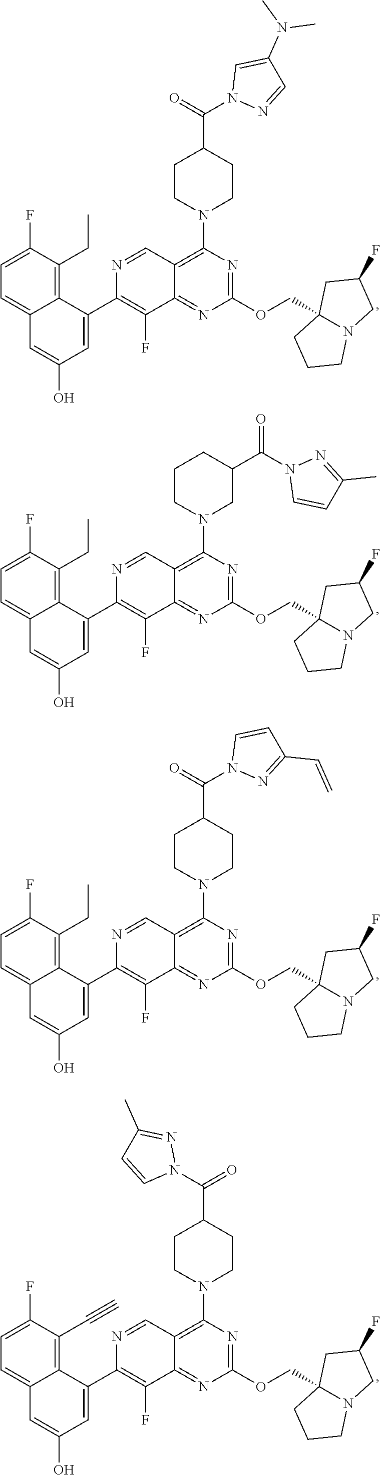 [SMILES: CCc1c(F)ccc2cc(O)cc(-c3ncc4c(N5CCC(C(=O)n6cc(N(C)C)cn6)CC5)nc(OC[C@@]56CCCN5C[C@H](F)C6)nc4c3F)c12 ; CCc1c(F)ccc2cc(O)cc(-c3ncc4c(N5CCCC(C(=O)n6ccc(C)n6)C5)nc(OC[C@@]56CCCN5C[C@H](F)C6)nc4c3F)c12 ; C=Cc1ccn(C(=O)C2CCN(c3nc(OC[C@@]45CCCN4C[C@H](F)C5)nc4c(F)c(-c5cc(O)cc6ccc(F)c(CC)c56)ncc34)CC2)n1 ; C#Cc1c(F)ccc2cc(O)cc(-c3ncc4c(N5CCC(C(=O)n6ccc(C)n6)CC5)nc(OC[C@@]56CCCN5C[C@H](F)C6)nc4c3F)c12]

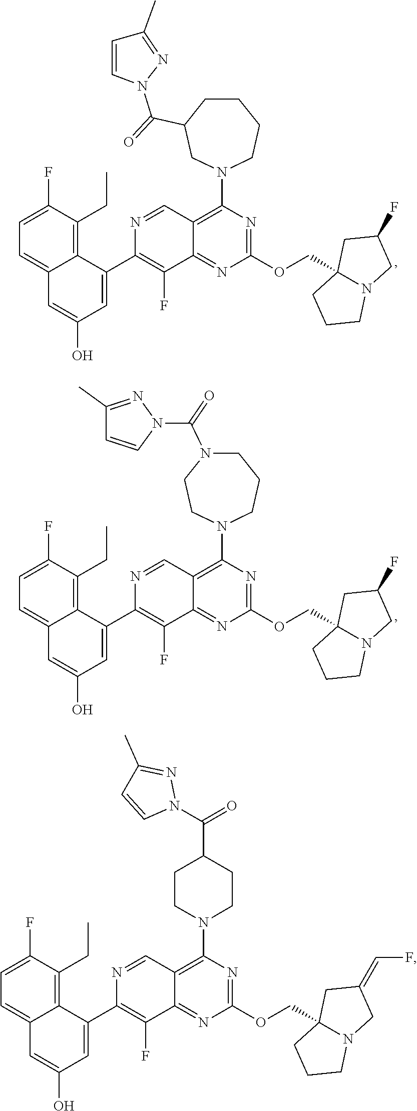

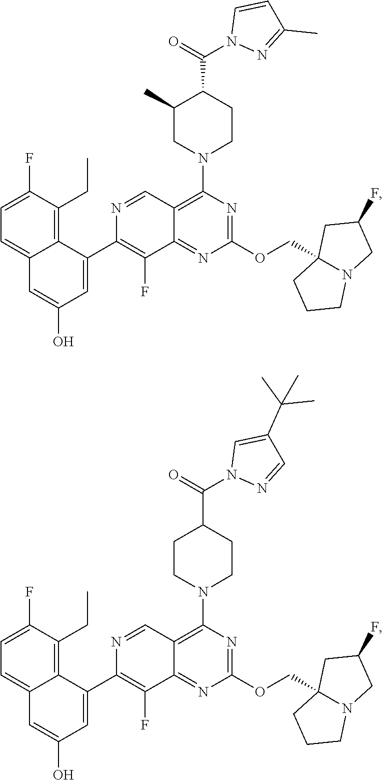 [SMILES: CCc1c(F)ccc2cc(O)cc(-c3ncc4c(N5CC[C@@H](C(=O)n6ccc(C)n6)[C@H](C)C5)nc(OC[C@@]56CCCN5C[C@H](F)C6)nc4c3F)c12 ; CCc1c(F)ccc2cc(O)cc(-c3ncc4c(N5CCC(C(=O)n6cc(C(C)(C)C)cn6)CC5)nc(OC[C@@]56CCCN5C[C@H](F)C6)nc4c3F)c12]

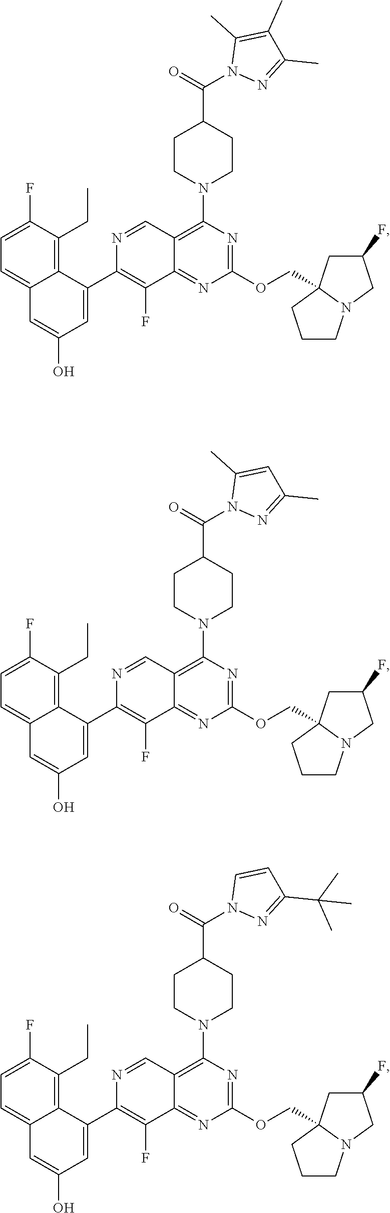 [SMILES: CCc1c(F)ccc2cc(O)cc(-c3ncc4c(N5CCC(C(=O)n6nc(C)c(C)c6C)CC5)nc(OC[C@@]56CCCN5C[C@H](F)C6)nc4c3F)c12 ; CCc1c(F)ccc2cc(O)cc(-c3ncc4c(N5CCC(C(=O)n6nc(C)cc6C)CC5)nc(OC[C@@]56CCCN5C[C@H](F)C6)nc4c3F)c12 ; CCc1c(F)ccc2cc(O)cc(-c3ncc4c(N5CCC(C(=O)n6ccc(C(C)(C)C)n6)CC5)nc(OC[C@@]56CCCN5C[C@H](F)C6)nc4c3F)c12]

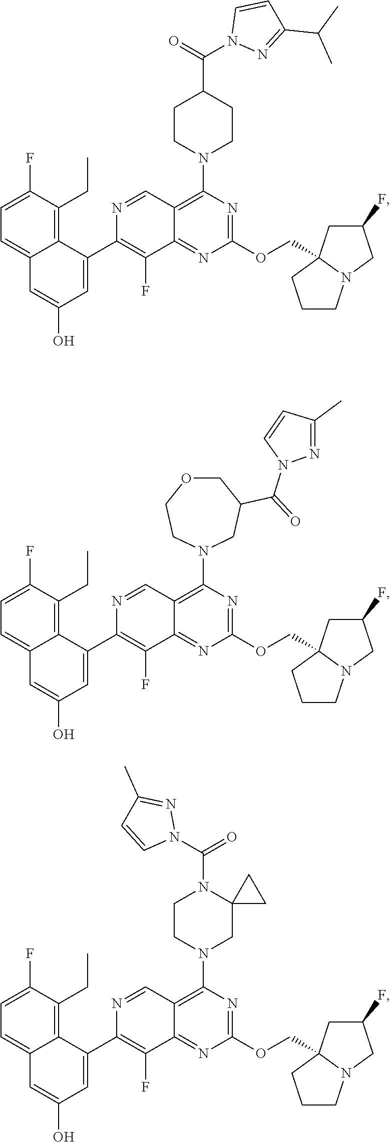 [SMILES: CCc1c(F)ccc2cc(O)cc(-c3ncc4c(N5CCC(C(=O)n6ccc(C(C)C)n6)CC5)nc(OC[C@@]56CCCN5C[C@H](F)C6)nc4c3F)c12 ; CCc1c(F)ccc2cc(O)cc(-c3ncc4c(N5CCOCC(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5CCN(C(=O)n6ccc(C)n6)C6(CC6)C5)nc(OC[C@@]56CCCN5C[C@H](F)C6)nc4c3F)c12]

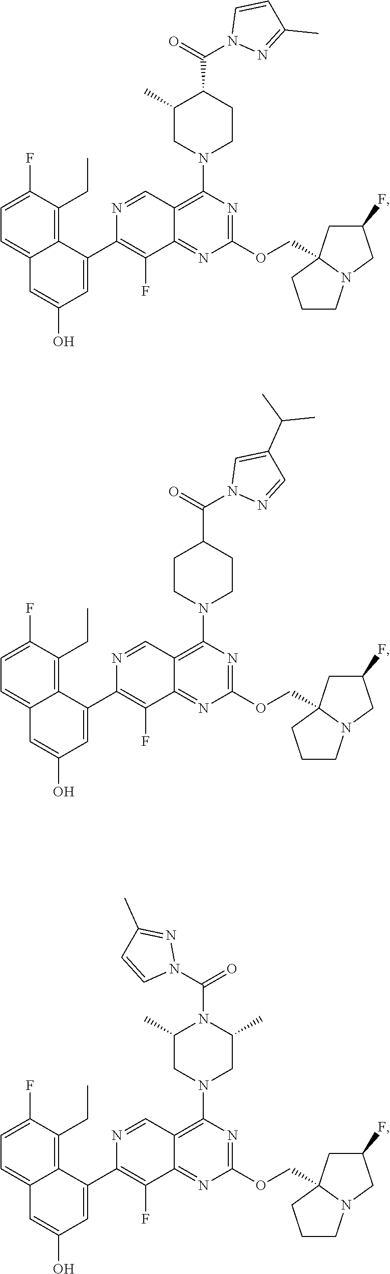 [SMILES: CCc1c(F)ccc2cc(O)cc(-c3ncc4c(N5CC[C@@H](C(=O)n6ccc(C)n6)[C@@H](C)C5)nc(OC[C@@]56CCCN5C[C@H](F)C6)nc4c3F)c12 ; CCc1c(F)ccc2cc(O)cc(-c3ncc4c(N5CCC(C(=O)n6cc(C(C)C)cn6)CC5)nc(OC[C@@]56CCCN5C[C@H](F)C6)nc4c3F)c12 ; CCc1c(F)ccc2cc(O)cc(-c3ncc4c(N5C[C@@H](C)N(C(=O)n6ccc(C)n6)[C@@H](C)C5)nc(OC[C@@]56CCCN5C[C@H](F)C6)nc4c3F)c12]

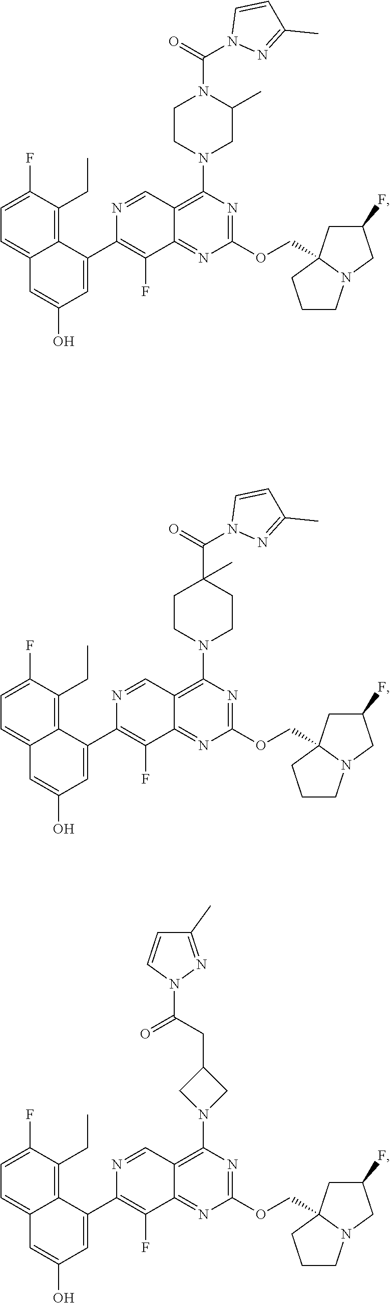 [SMILES: CCc1c(F)ccc2cc(O)cc(-c3ncc4c(N5CCN(C(=O)n6ccc(C)n6)C(C)C5)nc(OC[C@@]56CCCN5C[C@H](F)C6)nc4c3F)c12 ; CCc1c(F)ccc2cc(O)cc(-c3ncc4c(N5CCC(C)(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CC(CC(=O)n6ccc(C)n6)C5)nc(OC[C@@]56CCCN5C[C@H](F)C6)nc4c3F)c12]

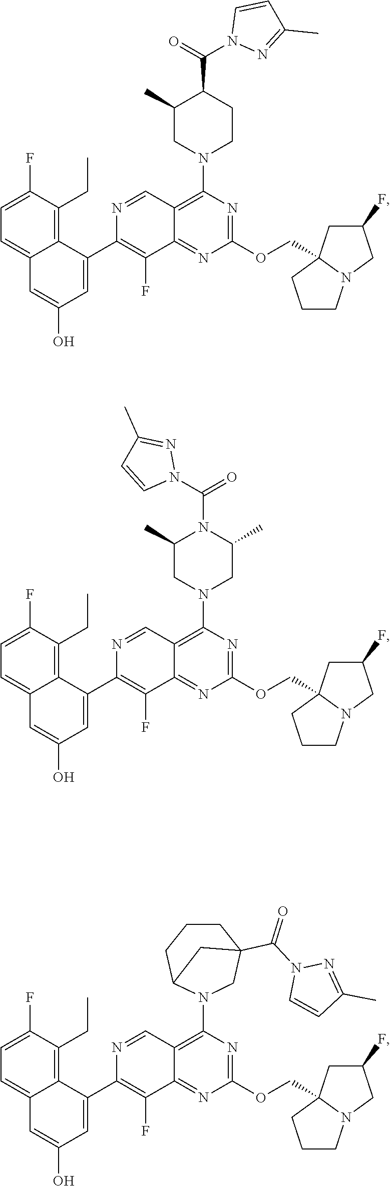 [SMILES: CCc1c(F)ccc2cc(O)cc(-c3ncc4c(N5CC[C@H](C(=O)n6ccc(C)n6)[C@H](C)C5)nc(OC[C@@]56CCCN5C[C@H](F)C6)nc4c3F)c12 ; CCc1c(F)ccc2cc(O)cc(-c3ncc4c(N5C[C@@H](C)N(C(=O)n6ccc(C)n6)[C@H](C)C5)nc(OC[C@@]56CCCN5C[C@H](F)C6)nc4c3F)c12 ; CCc1c(F)ccc2cc(O)cc(-c3ncc4c(N5CC6(C(=O)n7ccc(C)n7)CCCC5C6)nc(OC[C@@]56CCCN5C[C@H](F)C6)nc4c3F)c12]

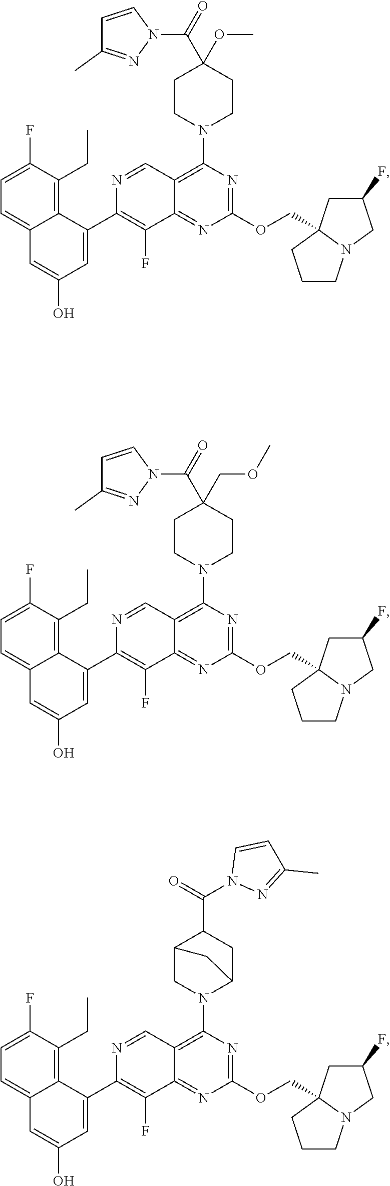 [SMILES: CCc1c(F)ccc2cc(O)cc(-c3ncc4c(N5CCC(OC)(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CCC(COC)(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CC6CC5CC6C(=O)n5ccc(C)n5)nc(OC[C@@]56CCCN5C[C@H](F)C6)nc4c3F)c12]

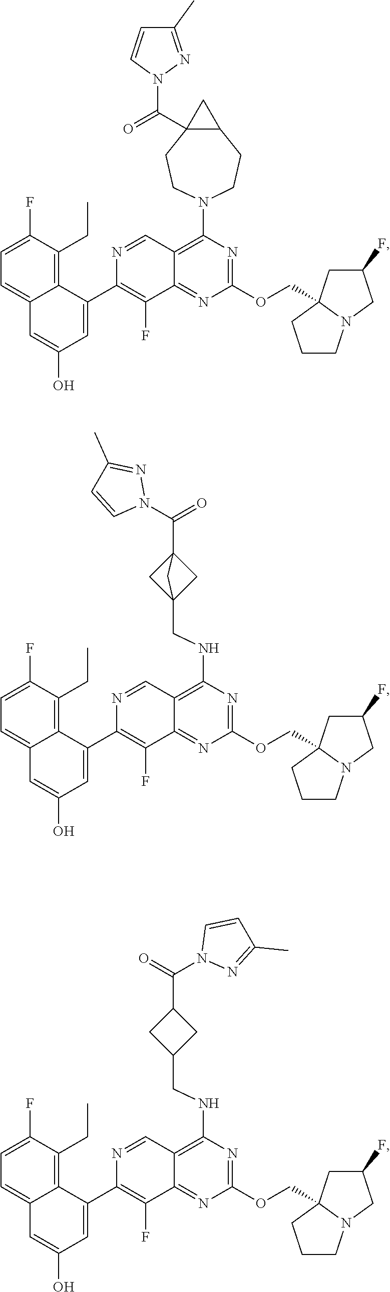 [SMILES: CCc1c(F)ccc2cc(O)cc(-c3ncc4c(N5CCC6CC6(C(=O)n6ccc(C)n6)CC5)nc(OC[C@@]56CCCN5C[C@H](F)C6)nc4c3F)c12 ; CCc1c(F)ccc2cc(O)cc(-c3ncc4c(NCC56CC(C(=O)n7ccc(C)n7)(C5)C6)nc(OC[C@@]56CCCN5C[C@H](F)C6)nc4c3F)c12 ; CCc1c(F)ccc2cc(O)cc(-c3ncc4c(NCC5CC(C(=O)n6ccc(C)n6)C5)nc(OC[C@@]56CCCN5C[C@H](F)C6)nc4c3F)c12]

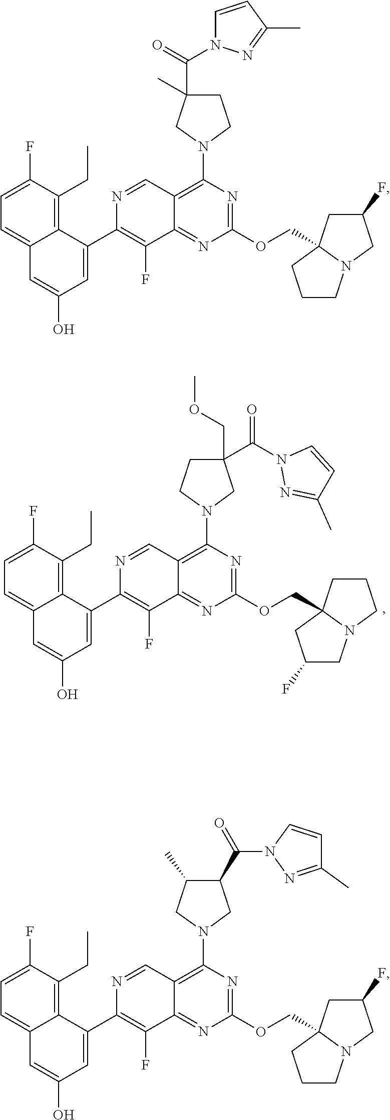 [SMILES: CCc1c(F)ccc2cc(O)cc(-c3ncc4c(N5CCC(C)(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5CCC(COC)(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5C[C@H](C(=O)n6ccc(C)n6)[C@@H](C)C5)nc(OC[C@@]56CCCN5C[C@H](F)C6)nc4c3F)c12]

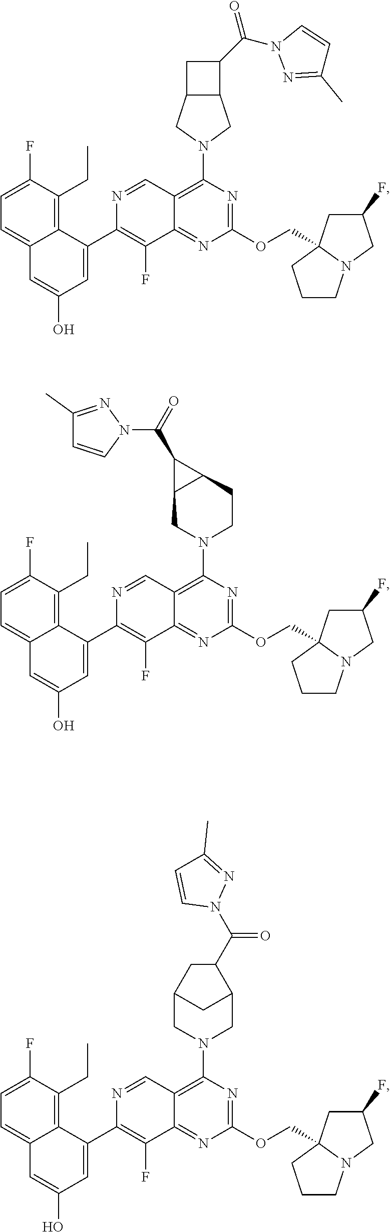 [SMILES: CCc1c(F)ccc2cc(O)cc(-c3ncc4c(N5CC6CC(C(=O)n7ccc(C)n7)C6C5)nc(OC[C@@]56CCCN5C[C@H](F)C6)nc4c3F)c12 ; CCc1c(F)ccc2cc(O)cc(-c3ncc4c(N5CC[C@H]6[C@@H](C5)[C@H]6C(=O)n5ccc(C)n5)nc(OC[C@@]56CCCN5C[C@H](F)C6)nc4c3F)c12 ; CCc1c(F)ccc2cc(O)cc(-c3ncc4c(N5CC6CC(C5)C(C(=O)n5ccc(C)n5)C6)nc(OC[C@@]56CCCN5C[C@H](F)C6)nc4c3F)c12]

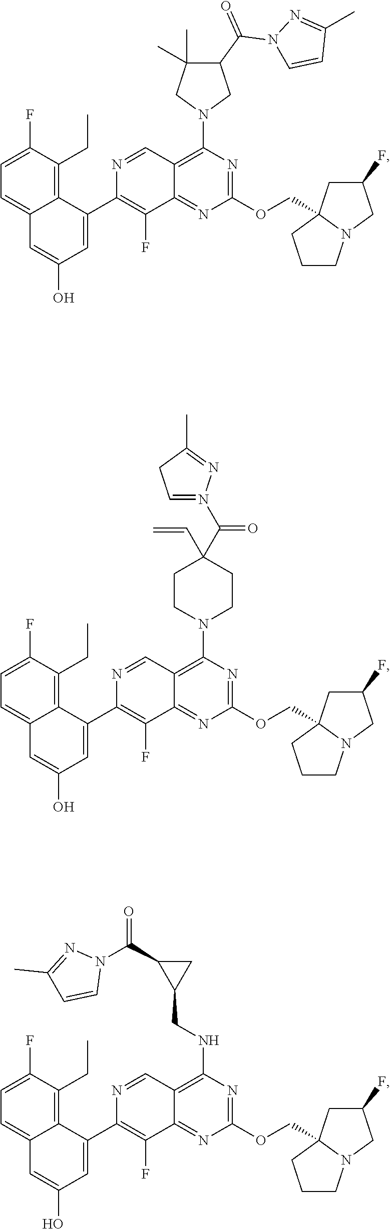

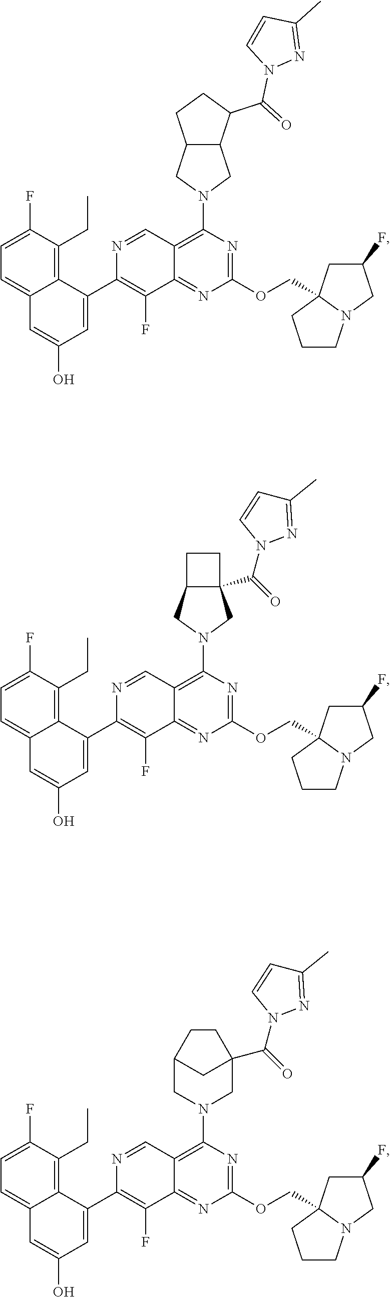 [SMILES: CCc1c(F)ccc2cc(O)cc(-c3ncc4c(N5CC6CCC(C(=O)n7ccc(C)n7)C6C5)nc(OC[C@@]56CCCN5C[C@H](F)C6)nc4c3F)c12 ; CCc1c(F)ccc2cc(O)cc(-c3ncc4c(N5C[C@H]6CC[C@@]6(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5CC6CCC(C(=O)n7ccc(C)n7)(C6)C5)nc(OC[C@@]56CCCN5C[C@H](F)C6)nc4c3F)c12]

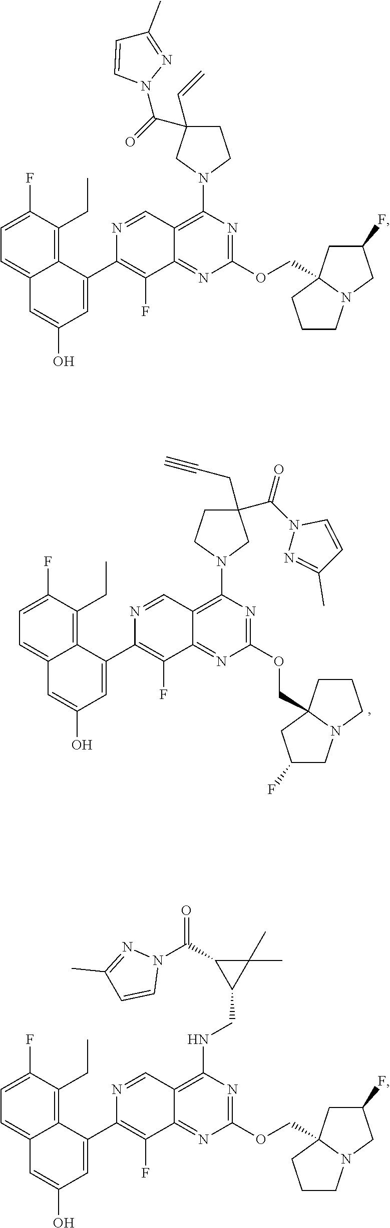 [SMILES: C=CC1(C(=O)n2ccc(C)n2)CCN(c2nc(OC[C@@]34CCCN3C[C@H](F)C4)nc3c(F)c(-c4cc(O)cc5ccc(F)c(CC)c45)ncc23)C1 ; C#CCC1(C(=O)n2ccc(C)n2)CCN(c2nc(OC[C@@]34CCCN3C[C@H](F)C4)nc3c(F)c(-c4cc(O)cc5ccc(F)c(CC)c45)ncc23)C1 ; CCc1c(F)ccc2cc(O)cc(-c3ncc4c(NC[C@@H]5[C@H](C(=O)n6ccc(C)n6)C5(C)C)nc(OC[C@@]56CCCN5C[C@H](F)C6)nc4c3F)c12]

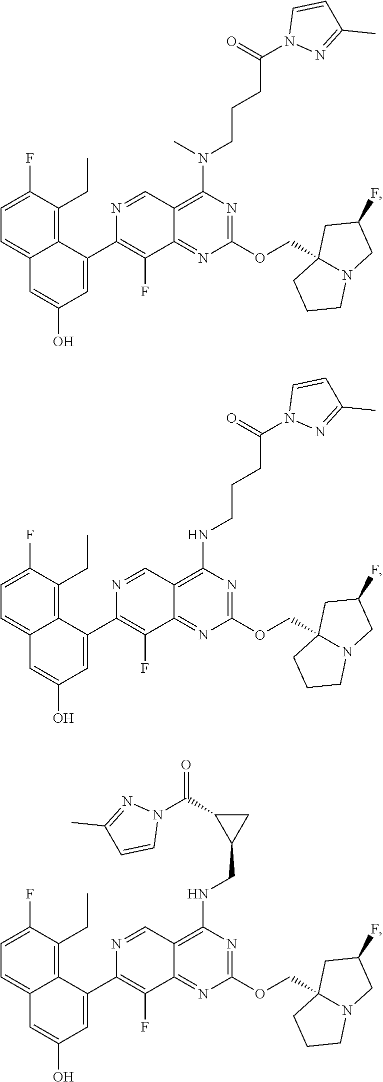 [SMILES: CCc1c(F)ccc2cc(O)cc(-c3ncc4c(N(C)CCCC(=O)n5ccc(C)n5)nc(OC[C@@]56CCCN5C[C@H](F)C6)nc4c3F)c12 ; CCc1c(F)ccc2cc(O)cc(-c3ncc4c(NCCCC(=O)n5ccc(C)n5)nc(OC[C@@]56CCCN5C[C@H](F)C6)nc4c3F)c12 ; CCc1c(F)ccc2cc(O)cc(-c3ncc4c(NC[C@@H]5C[C@H]5C(=O)n5ccc(C)n5)nc(OC[C@@]56CCCN5C[C@H](F)C6)nc4c3F)c12]

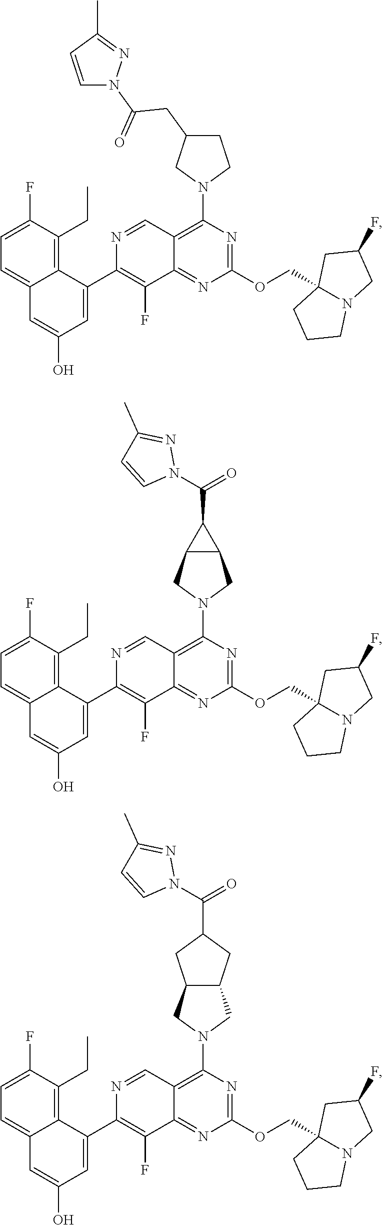 [SMILES: CCc1c(F)ccc2cc(O)cc(-c3ncc4c(N5CCC(CC(=O)n6ccc(C)n6)C5)nc(OC[C@@]56CCCN5C[C@H](F)C6)nc4c3F)c12 ; CCc1c(F)ccc2cc(O)cc(-c3ncc4c(N5C[C@@H]6[C@H](C5)[C@@H]6C(=O)n5ccc(C)n5)nc(OC[C@@]56CCCN5C[C@H](F)C6)nc4c3F)c12 ; CCc1c(F)ccc2cc(O)cc(-c3ncc4c(N5C[C@H]6CC(C(=O)n7ccc(C)n7)C[C@@H]6C5)nc(OC[C@@]56CCCN5C[C@H](F)C6)nc4c3F)c12]

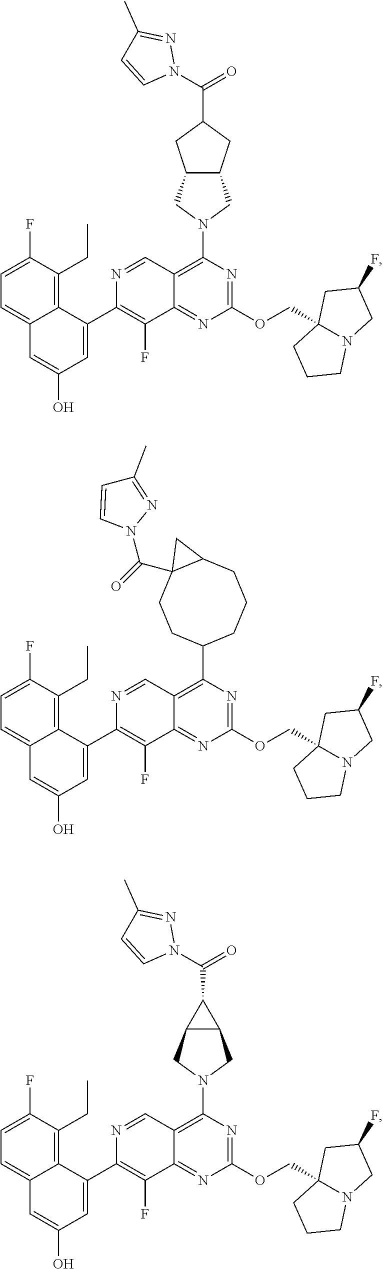 [SMILES: CCc1c(F)ccc2cc(O)cc(-c3ncc4c(N5C[C@H]6CC(C(=O)n7ccc(C)n7)C[C@H]6C5)nc(OC[C@@]56CCCN5C[C@H](F)C6)nc4c3F)c12 ; CCc1c(F)ccc2cc(O)cc(-c3ncc4c(C5CCCC6CC6(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5C[C@@H]6[C@H](C5)[C@H]6C(=O)n5ccc(C)n5)nc(OC[C@@]56CCCN5C[C@H](F)C6)nc4c3F)c12]

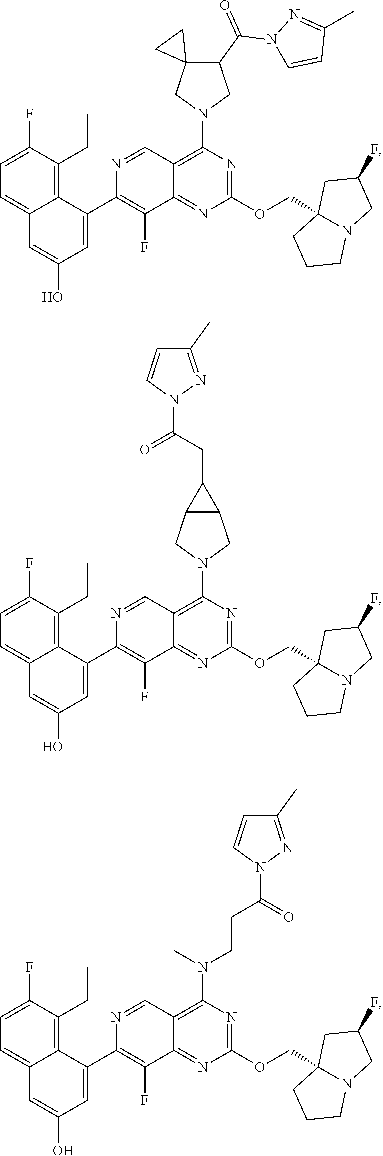 [SMILES: CCc1c(F)ccc2cc(O)cc(-c3ncc4c(N5CC(C(=O)n6ccc(C)n6)C6(CC6)C5)nc(OC[C@@]56CCCN5C[C@H](F)C6)nc4c3F)c12 ; CCc1c(F)ccc2cc(O)cc(-c3ncc4c(N5CC6C(CC(=O)n7ccc(C)n7)C6C5)nc(OC[C@@]56CCCN5C[C@H](F)C6)nc4c3F)c12 ; CCc1c(F)ccc2cc(O)cc(-c3ncc4c(N(C)CCC(=O)n5ccc(C)n5)nc(OC[C@@]56CCCN5C[C@H](F)C6)nc4c3F)c12]

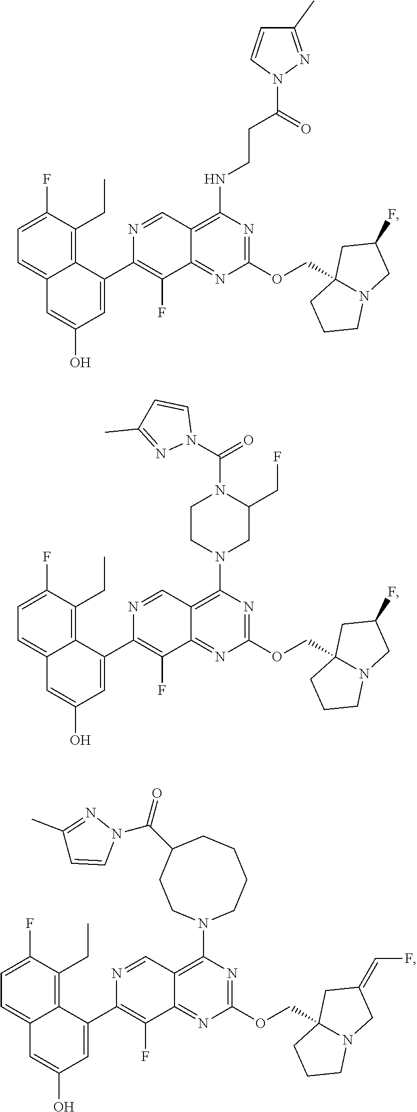 [SMILES: CCc1c(F)ccc2cc(O)cc(-c3ncc4c(NCCC(=O)n5ccc(C)n5)nc(OC[C@@]56CCCN5C[C@H](F)C6)nc4c3F)c12 ; CCc1c(F)ccc2cc(O)cc(-c3ncc4c(N5CCN(C(=O)n6ccc(C)n6)C(CF)C5)nc(OC[C@@]56CCCN5C[C@H](F)C6)nc4c3F)c12 ; CCc1c(F)ccc2cc(O)cc(-c3ncc4c(N5CCCCC(C(=O)n6ccc(C)n6)CC5)nc(OC[C@@]56CCCN5C/C(=C\F)C6)nc4c3F)c12]

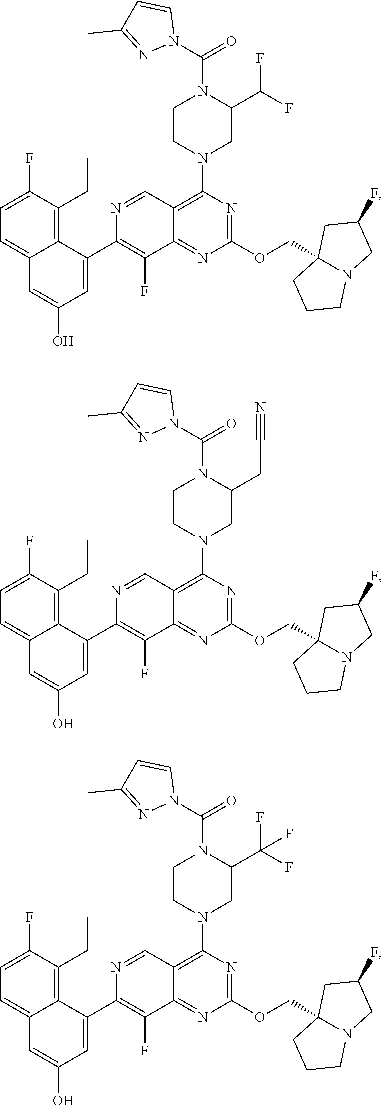 [SMILES: CCc1c(F)ccc2cc(O)cc(-c3ncc4c(N5CCN(C(=O)n6ccc(C)n6)C(C(F)F)C5)nc(OC[C@@]56CCCN5C[C@H](F)C6)nc4c3F)c12 ; CCc1c(F)ccc2cc(O)cc(-c3ncc4c(N5CCN(C(=O)n6ccc(C)n6)C(CC#N)C5)nc(OC[C@@]56CCCN5C[C@H](F)C6)nc4c3F)c12 ; CCc1c(F)ccc2cc(O)cc(-c3ncc4c(N5CCN(C(=O)n6ccc(C)n6)C(C(F)(F)F)C5)nc(OC[C@@]56CCCN5C[C@H](F)C6)nc4c3F)c12]

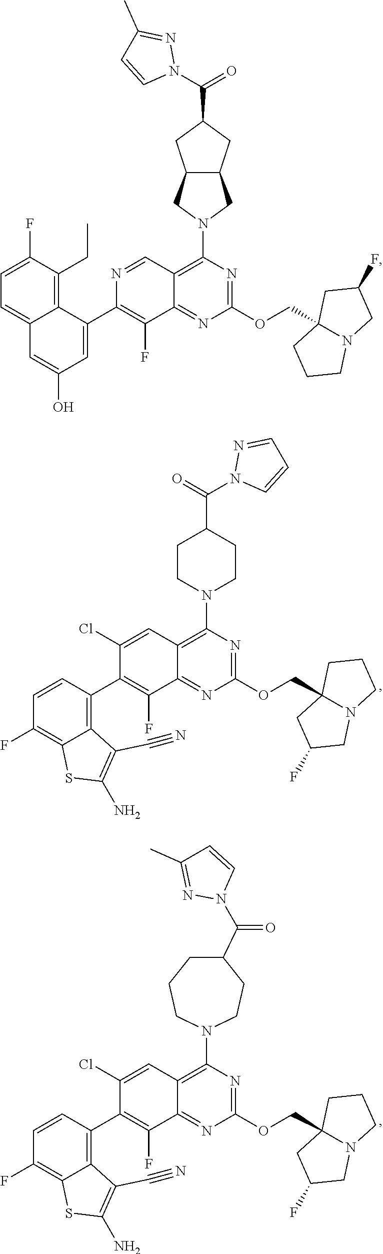 [SMILES: CCc1c(F)ccc2cc(O)cc(-c3ncc4c(N5C[C@H]6C[C@@H](C(=O)n7ccc(C)n7)C[C@H]6C5)nc(OC[C@@]56CCCN5C[C@H](F)C6)nc4c3F)c12 ; N#Cc1c(N)sc2c(F)ccc(-c3c(Cl)cc4c(N5CCC(C(=O)n6cccn6)CC5)nc(OC[C@@]56CCCN5C[C@H](F)C6)nc4c3F)c12 ; Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

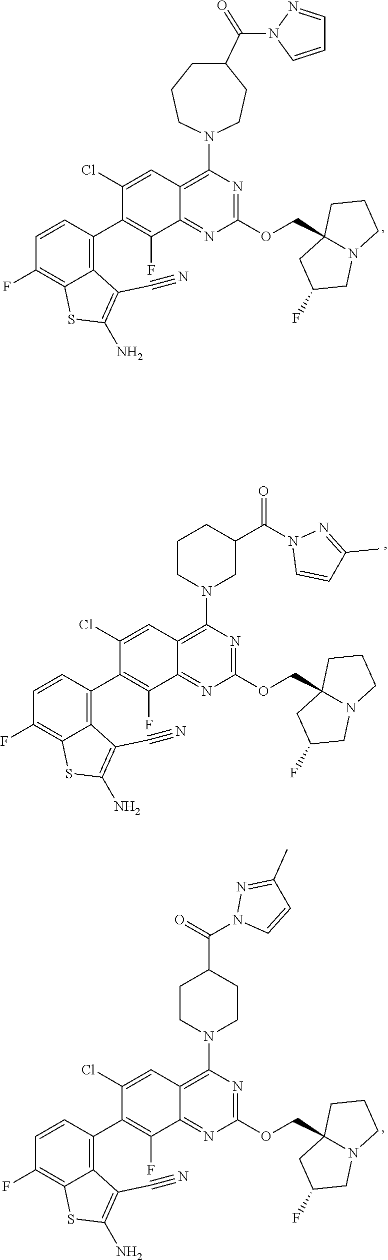 [SMILES: N#Cc1c(N)sc2c(F)ccc(-c3c(Cl)cc4c(N5CCCC(C(=O)n6cccn6)CC5)nc(OC[C@@]56CCCN5C[C@H](F)C6)nc4c3F)c12 ; Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)C2)n1 ; Cc1ccn(C(=O)C2CCN(c3nc(OC[C@@]45CCCN4C[C@H](F)C5)nc4c(F)c(-c5ccc(F)c6sc(N)c(C#N)c56)c(Cl)cc34)CC2)n1]

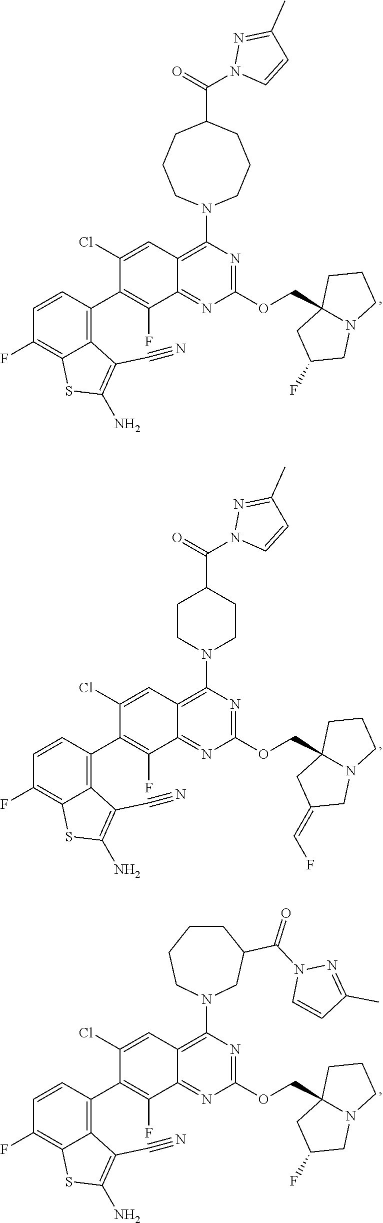 [SMILES: Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CCC2)n1 ; Cc1ccn(C(=O)C2CCN(c3nc(OC[C@@]45CCCN4C/C(=C\F)C5)nc4c(F)c(-c5ccc(F)c6sc(N)c(C#N)c56)c(Cl)cc34)CC2)n1 ; Cc1ccn(C(=O)C2CCCCN(c3nc(OC[C@@]45CCCN4C[C@H](F)C5)nc4c(F)c(-c5ccc(F)c6sc(N)c(C#N)c56)c(Cl)cc34)C2)n1]

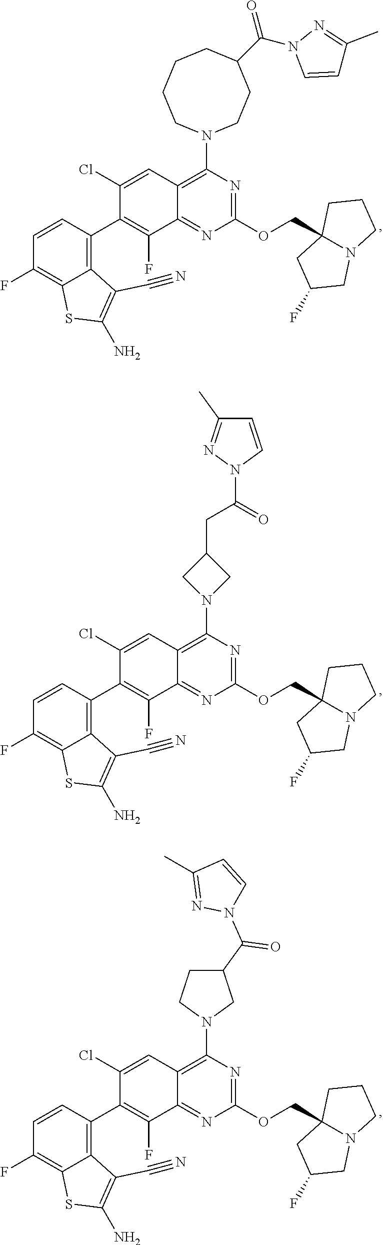 [SMILES: Cc1ccn(C(=O)C2CCCCN(c3nc(OC[C@@]45CCCN4C[C@H](F)C5)nc4c(F)c(-c5ccc(F)c6sc(N)c(C#N)c56)c(Cl)cc34)CC2)n1 ; Cc1ccn(C(=O)CC2CN(c3nc(OC[C@@]45CCCN4C[C@H](F)C5)nc4c(F)c(-c5ccc(F)c6sc(N)c(C#N)c56)c(Cl)cc34)C2)n1 ; Cc1ccn(C(=O)C2CCN(c3nc(OC[C@@]45CCCN4C[C@H](F)C5)nc4c(F)c(-c5ccc(F)c6sc(N)c(C#N)c56)c(Cl)cc34)C2)n1]

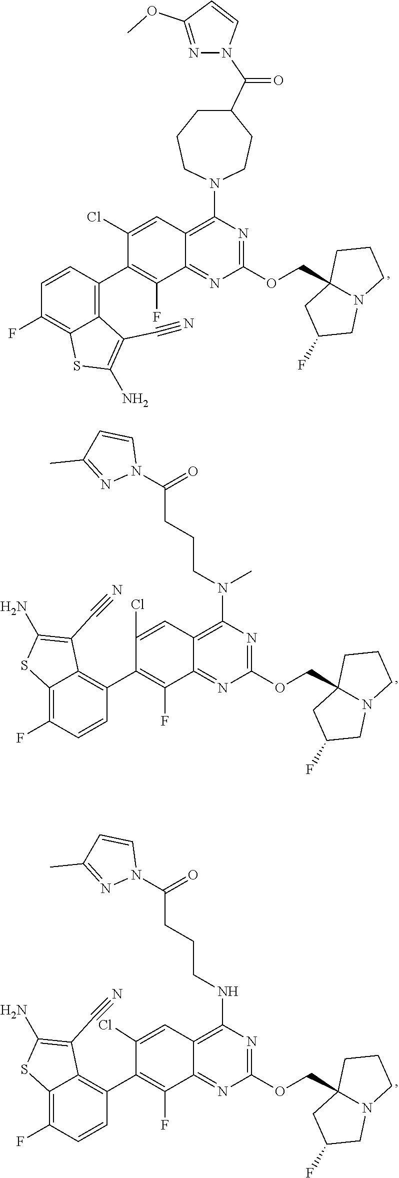

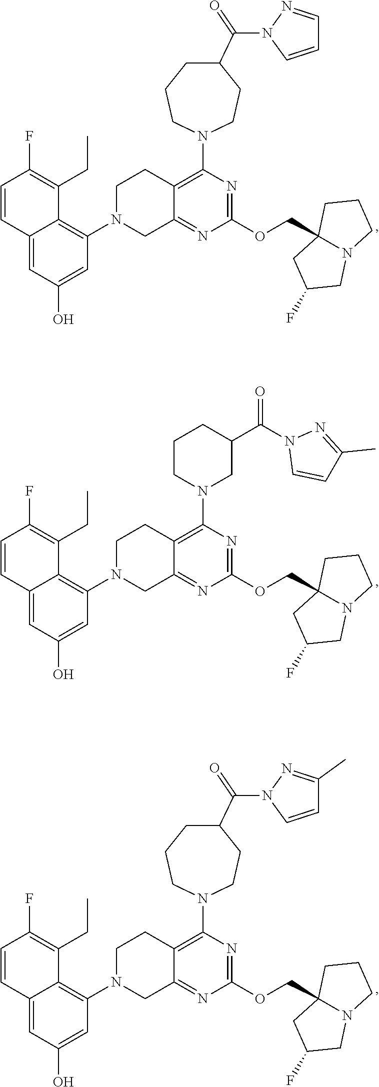 [SMILES: CCc1c(F)ccc2cc(O)cc(N3CCc4c(nc(OC[C@@]56CCCN5C[C@H](F)C6)nc4N4CCCC(C(=O)n5cccn5)CC4)C3)c12 ; CCc1c(F)ccc2cc(O)cc(N3CCc4c(nc(OC[C@@]56CCCN5C[C@H](F)C6)nc4N4CCCC(C(=O)n5ccc(C)n5)C4)C3)c12 ; CCc1c(F)ccc2cc(O)cc(N3CCc4c(nc(OC[C@@]56CCCN5C[C@H](F)C6)nc4N4CCCC(C(=O)n5ccc(C)n5)CC4)C3)c12]

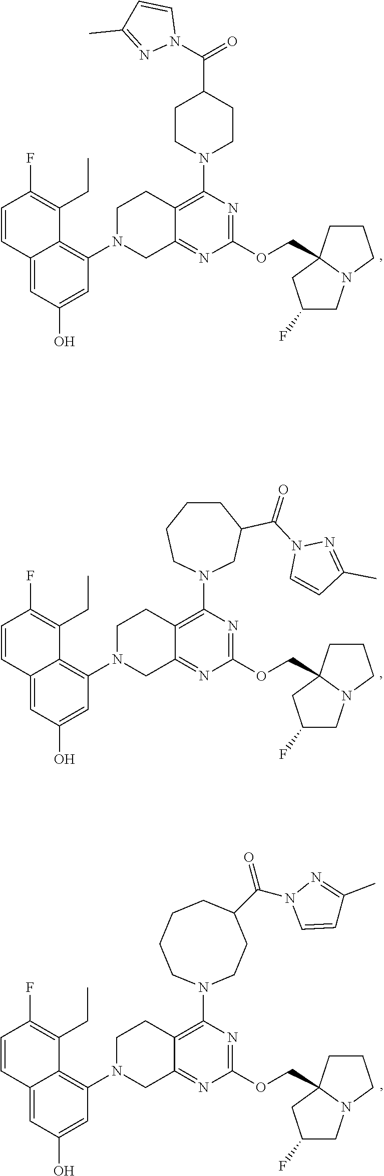 [SMILES: CCc1c(F)ccc2cc(O)cc(N3CCc4c(nc(OC[C@@]56CCCN5C[C@H](F)C6)nc4N4CCC(C(=O)n5ccc(C)n5)CC4)C3)c12 ; CCc1c(F)ccc2cc(O)cc(N3CCc4c(nc(OC[C@@]56CCCN5C[C@H](F)C6)nc4N4CCCCC(C(=O)n5ccc(C)n5)C4)C3)c12 ; CCc1c(F)ccc2cc(O)cc(N3CCc4c(nc(OC[C@@]56CCCN5C[C@H](F)C6)nc4N4CCCCC(C(=O)n5ccc(C)n5)CC4)C3)c12]

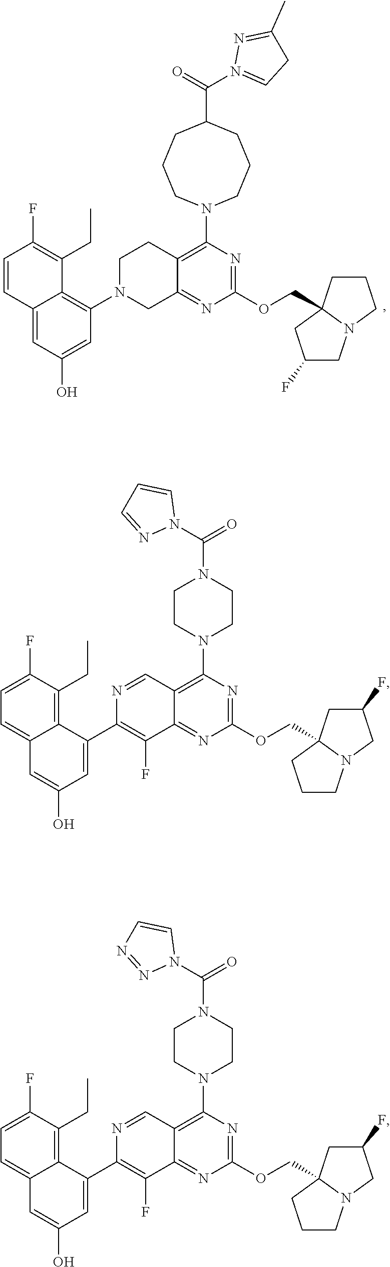

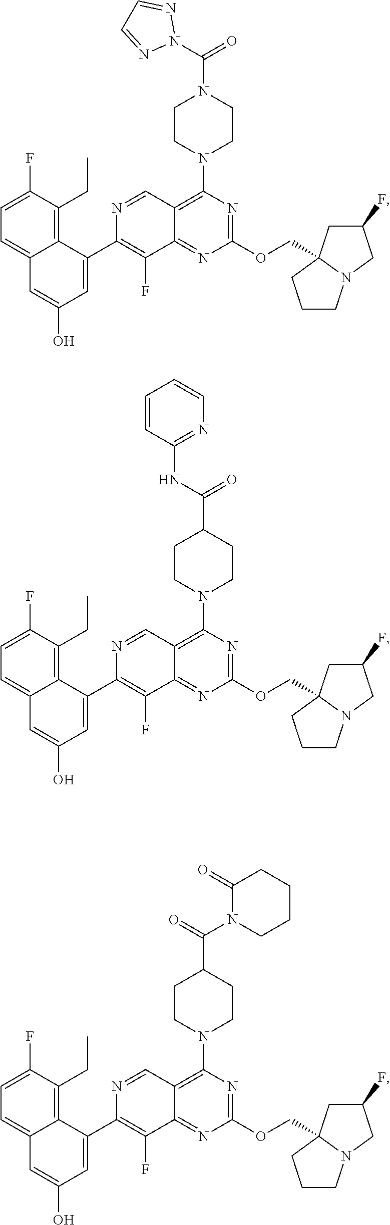 [SMILES: CCc1c(F)ccc2cc(O)cc(-c3ncc4c(N5CCN(C(=O)n6nccn6)CC5)nc(OC[C@@]56CCCN5C[C@H](F)C6)nc4c3F)c12 ; CCc1c(F)ccc2cc(O)cc(-c3ncc4c(N5CCC(C(=O)Nc6ccccn6)CC5)nc(OC[C@@]56CCCN5C[C@H](F)C6)nc4c3F)c12 ; CCc1c(F)ccc2cc(O)cc(-c3ncc4c(N5CCC(C(=O)N6CCCCC6=O)CC5)nc(OC[C@@]56CCCN5C[C@H](F)C6)nc4c3F)c12]

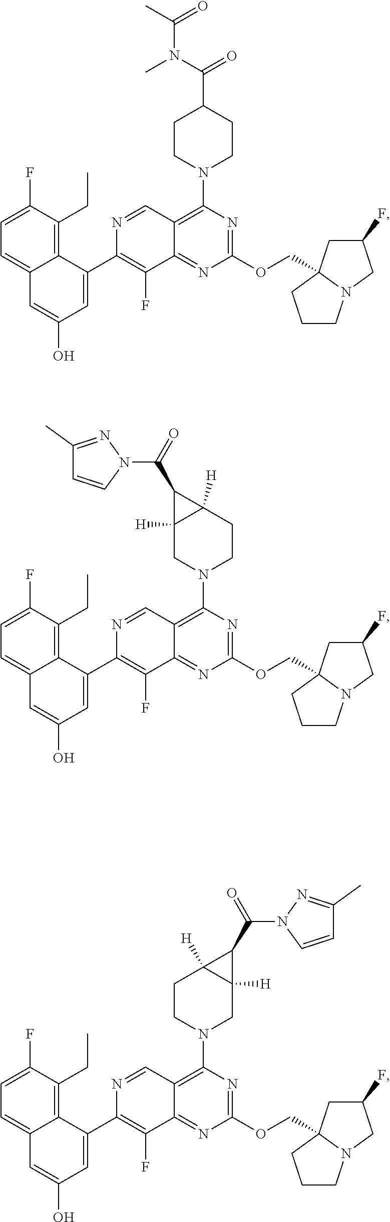 [SMILES: CCc1c(F)ccc2cc(O)cc(-c3ncc4c(N5CCC(C(=O)N(C)C(C)=O)CC5)nc(OC[C@@]56CCCN5C[C@H](F)C6)nc4c3F)c12 ; CCc1c(F)ccc2cc(O)cc(-c3ncc4c(N5CC[C@H]6[C@@H](C5)[C@H]6C(=O)n5ccc(C)n5)nc(OC[C@@]56CCCN5C[C@H](F)C6)nc4c3F)c12 ; CCc1c(F)ccc2cc(O)cc(-c3ncc4c(N5CC[C@@H]6[C@H](C5)[C@@H]6C(=O)n5ccc(C)n5)nc(OC[C@@]56CCCN5C[C@H](F)C6)nc4c3F)c12]

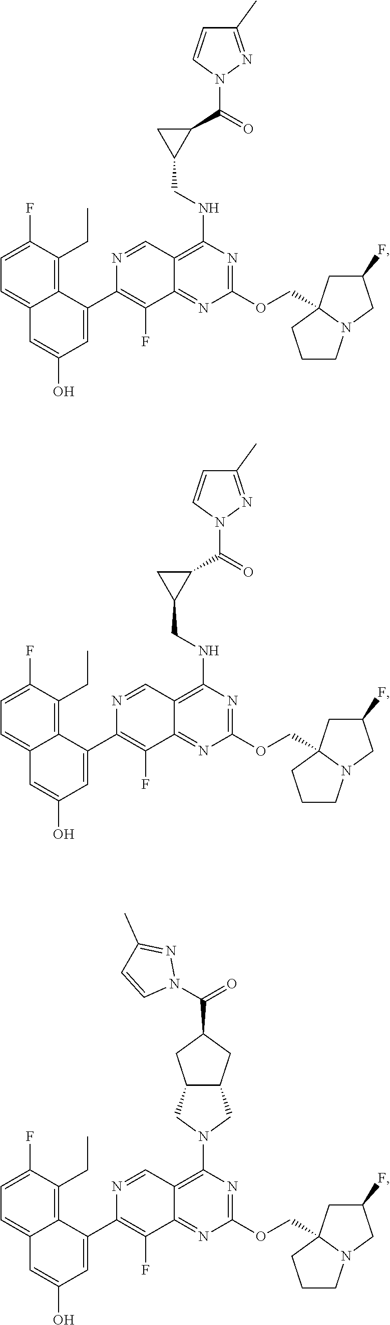 [SMILES: CCc1c(F)ccc2cc(O)cc(-c3ncc4c(NC[C@@H]5C[C@H]5C(=O)n5ccc(C)n5)nc(OC[C@@]56CCCN5C[C@H](F)C6)nc4c3F)c12 ; CCc1c(F)ccc2cc(O)cc(-c3ncc4c(NC[C@H]5C[C@@H]5C(=O)n5ccc(C)n5)nc(OC[C@@]56CCCN5C[C@H](F)C6)nc4c3F)c12 ; CCc1c(F)ccc2cc(O)cc(-c3ncc4c(N5C[C@H]6C[C@H](C(=O)n7ccc(C)n7)C[C@H]6C5)nc(OC[C@@]56CCCN5C[C@H](F)C6)nc4c3F)c12]

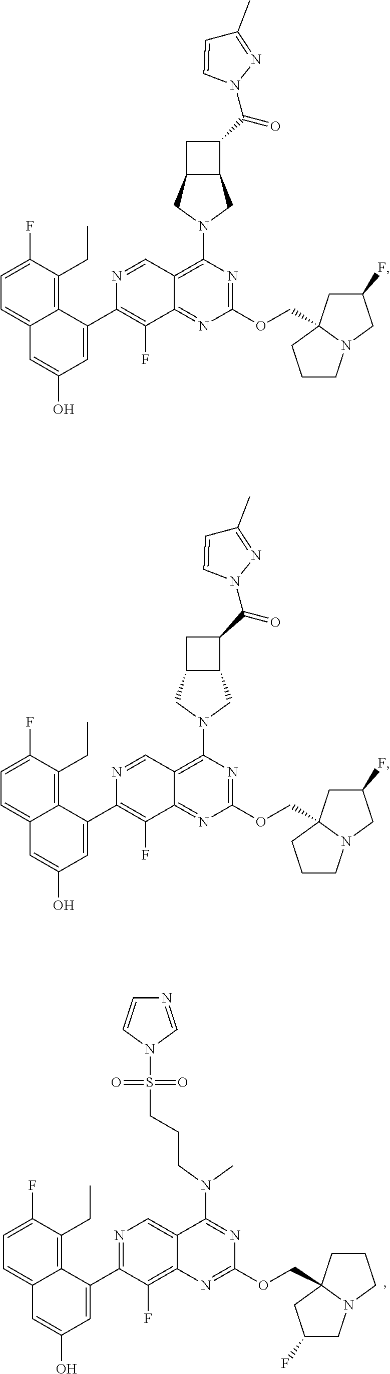 [SMILES: CCc1c(F)ccc2cc(O)cc(-c3ncc4c(N5C[C@H]6C[C@H](C(=O)n7ccc(C)n7)[C@H]6C5)nc(OC[C@@]56CCCN5C[C@H](F)C6)nc4c3F)c12 ; CCc1c(F)ccc2cc(O)cc(-c3ncc4c(N5C[C@@H]6C[C@@H](C(=O)n7ccc(C)n7)[C@@H]6C5)nc(OC[C@@]56CCCN5C[C@H](F)C6)nc4c3F)c12 ; CCc1c(F)ccc2cc(O)cc(-c3ncc4c(N(C)CCCS(=O)(=O)n5ccnc5)nc(OC[C@@]56CCCN5C[C@H](F)C6)nc4c3F)c12]

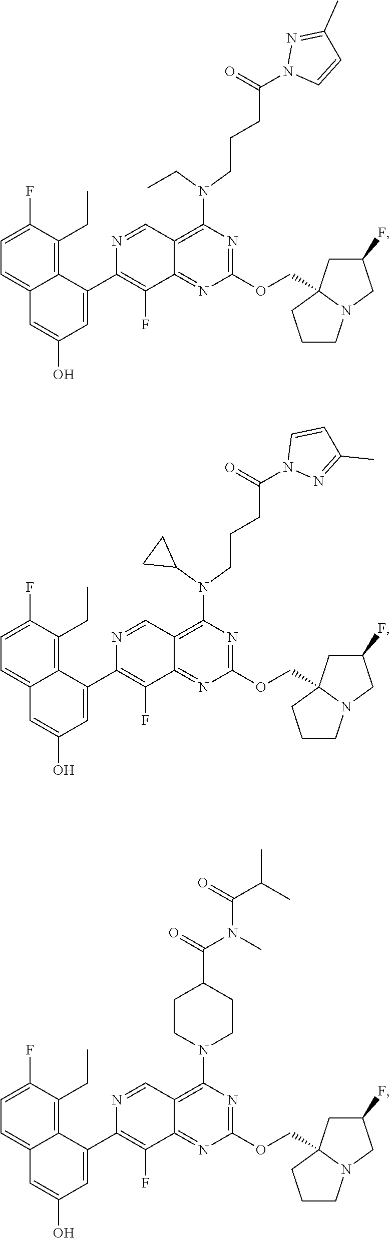 [SMILES: CCc1c(F)ccc2cc(O)cc(-c3ncc4c(N(CC)CCCC(=O)n5ccc(C)n5)nc(OC[C@@]56CCCN5C[C@H](F)C6)nc4c3F)c12 ; CCc1c(F)ccc2cc(O)cc(-c3ncc4c(N(CCCC(=O)n5ccc(C)n5)C5CC5)nc(OC[C@@]56CCCN5C[C@H](F)C6)nc4c3F)c12 ; CCc1c(F)ccc2cc(O)cc(-c3ncc4c(N5CCC(C(=O)N(C)C(=O)C(C)C)CC5)nc(OC[C@@]56CCCN5C[C@H](F)C6)nc4c3F)c12]

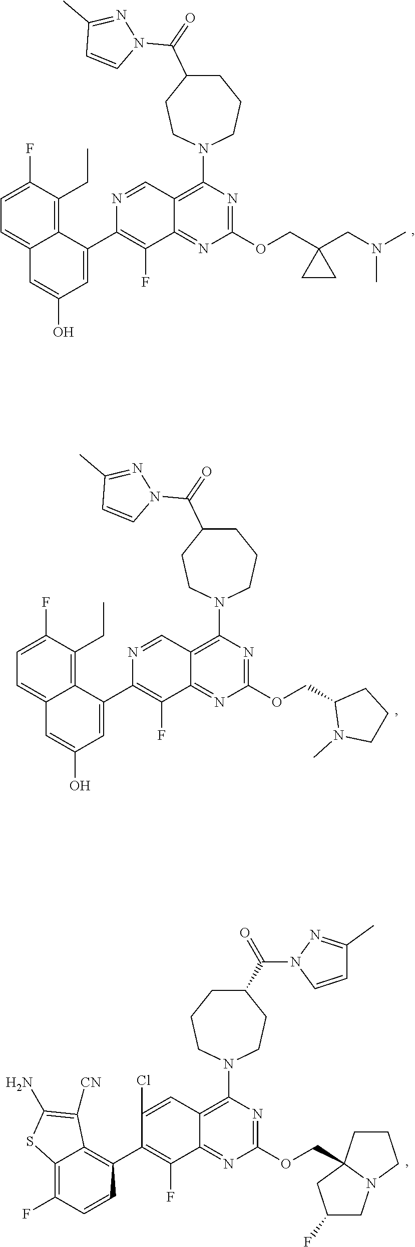

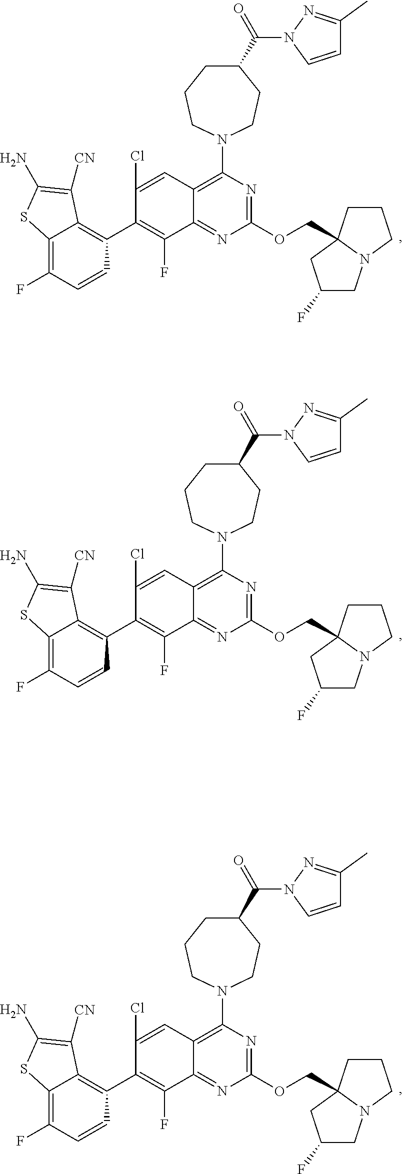

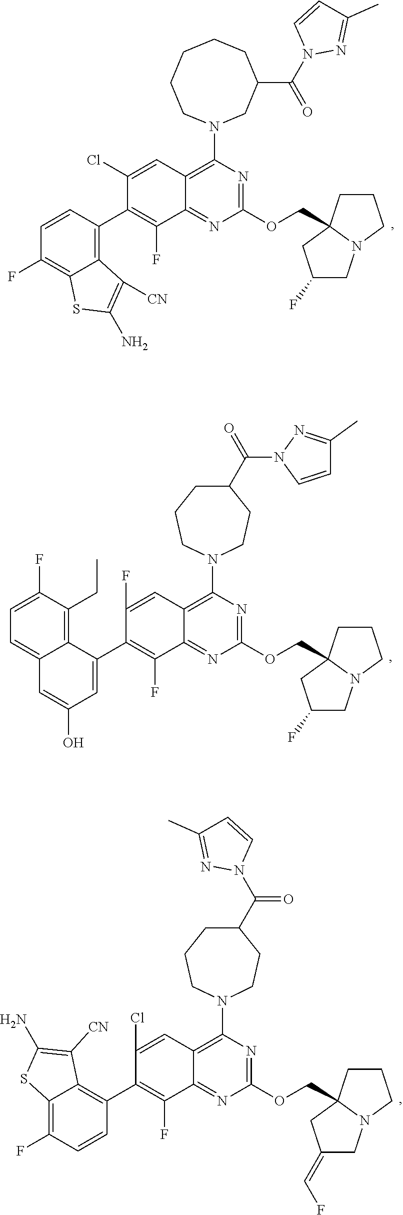

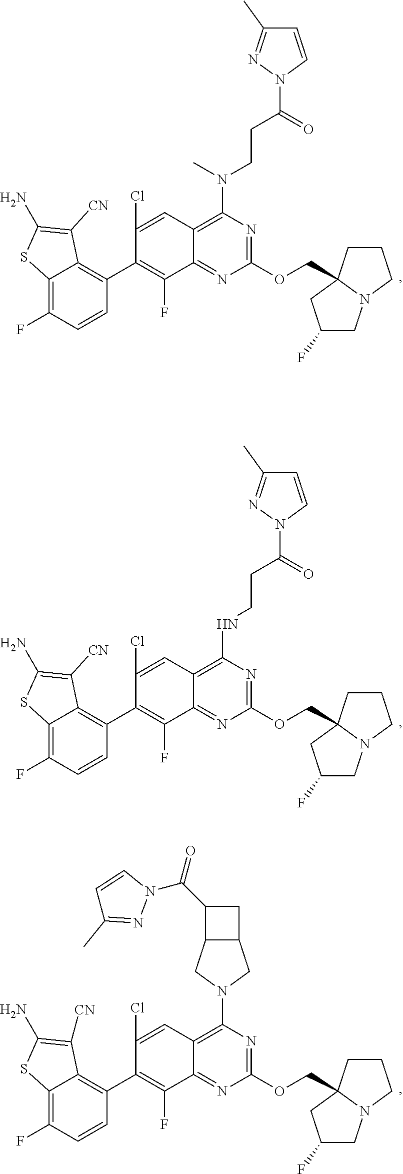

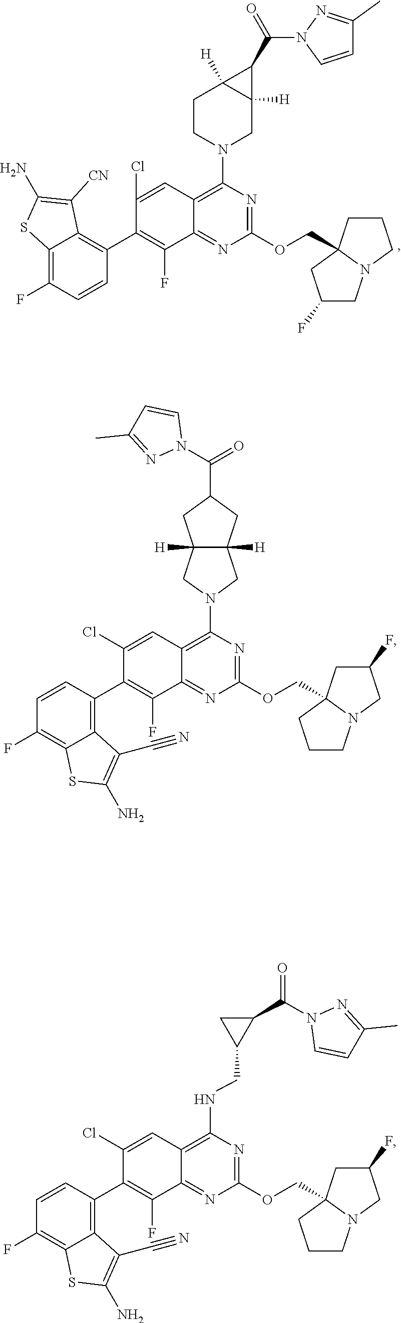 [SMILES: Cc1ccn(C(=O)[C@@H]2[C@@H]3CCN(c4nc(OC[C@@]56CCCN5C[C@H](F)C6)nc5c(F)c(-c6ccc(F)c7sc(N)c(C#N)c67)c(Cl)cc45)C[C@@H]32)n1 ; Cc1ccn(C(=O)C2C[C@@H]3CN(c4nc(OC[C@@]56CCCN5C[C@H](F)C6)nc5c(F)c(-c6ccc(F)c7sc(N)c(C#N)c67)c(Cl)cc45)C[C@@H]3C2)n1 ; Cc1ccn(C(=O)[C@@H]2C[C@H]2CNc2nc(OC[C@@]34CCCN3C[C@H](F)C4)nc3c(F)c(-c4ccc(F)c5sc(N)c(C#N)c45)c(Cl)cc23)n1]

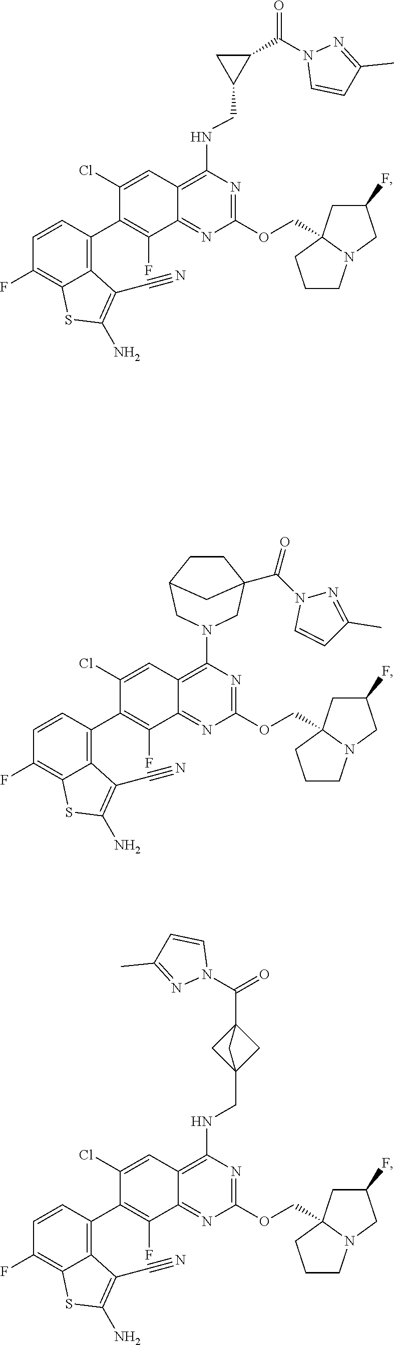 [SMILES: Cc1ccn(C(=O)[C@H]2C[C@H]2CNc2nc(OC[C@@]34CCCN3C[C@H](F)C4)nc3c(F)c(-c4ccc(F)c5sc(N)c(C#N)c45)c(Cl)cc23)n1 ; Cc1ccn(C(=O)C23CCC(CN(c4nc(OC[C@@]56CCCN5C[C@H](F)C6)nc5c(F)c(-c6ccc(F)c7sc(N)c(C#N)c67)c(Cl)cc45)C2)C3)n1 ; Cc1ccn(C(=O)C23CC(CNc4nc(OC[C@@]56CCCN5C[C@H](F)C6)nc5c(F)c(-c6ccc(F)c7sc(N)c(C#N)c67)c(Cl)cc45)(C2)C3)n1]

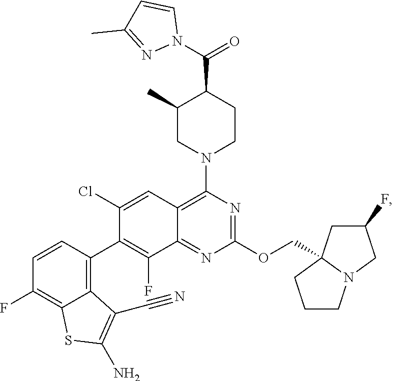 [SMILES: Cc1ccn(C(=O)[C@H]2CCN(c3nc(OC[C@@]45CCCN4C[C@H](F)C5)nc4c(F)c(-c5ccc(F)c6sc(N)c(C#N)c56)c(Cl)cc34)C[C@H]2C)n1]

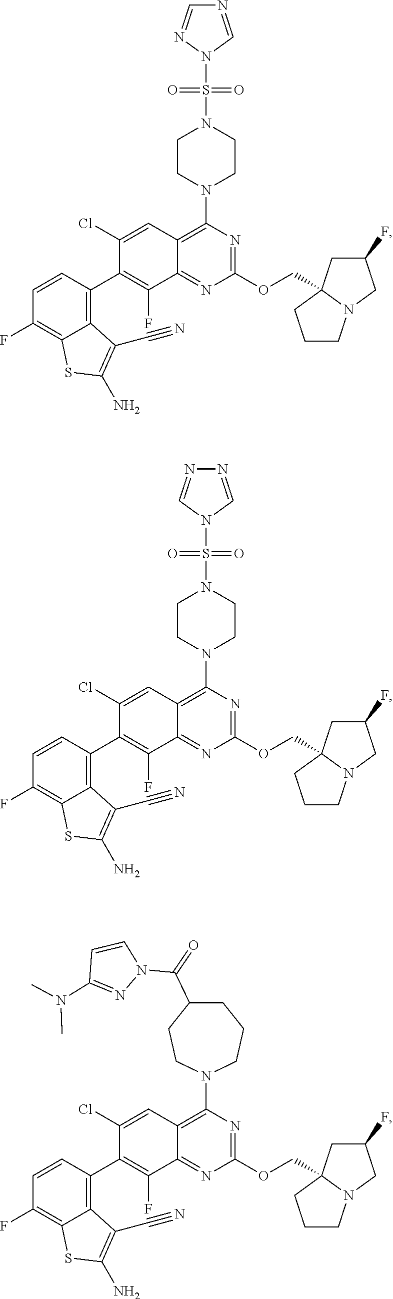 [SMILES: N#Cc1c(N)sc2c(F)ccc(-c3c(Cl)cc4c(N5CCN(S(=O)(=O)n6cncn6)CC5)nc(OC[C@@]56CCCN5C[C@H](F)C6)nc4c3F)c12 ; N#Cc1c(N)sc2c(F)ccc(-c3c(Cl)cc4c(N5CCN(S(=O)(=O)n6cnnc6)CC5)nc(OC[C@@]56CCCN5C[C@H](F)C6)nc4c3F)c12 ; CN(C)c1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

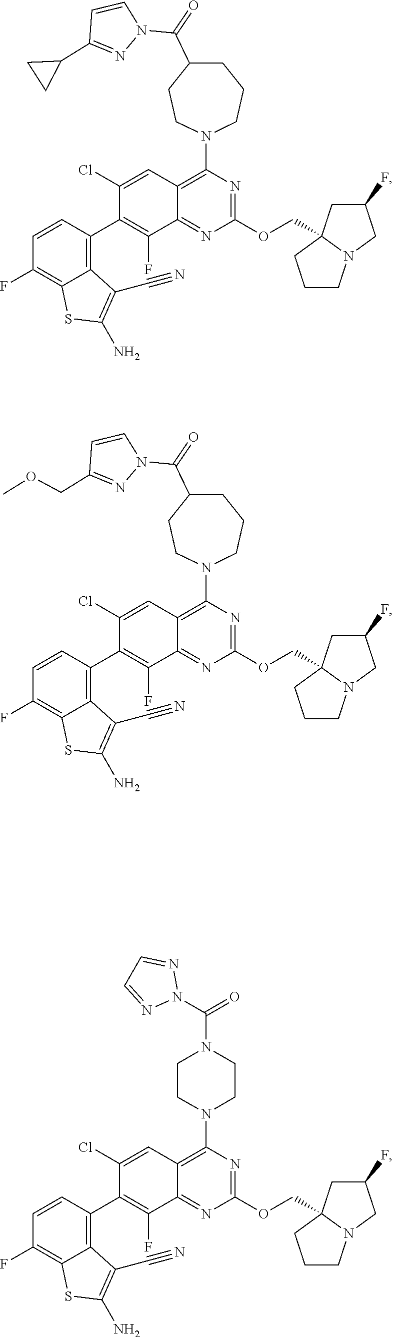 [SMILES: N#Cc1c(N)sc2c(F)ccc(-c3c(Cl)cc4c(N5CCCC(C(=O)n6ccc(C7CC7)n6)CC5)nc(OC[C@@]56CCCN5C[C@H](F)C6)nc4c3F)c12 ; COCc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; N#Cc1c(N)sc2c(F)ccc(-c3c(Cl)cc4c(N5CCN(C(=O)n6nccn6)CC5)nc(OC[C@@]56CCCN5C[C@H](F)C6)nc4c3F)c12]

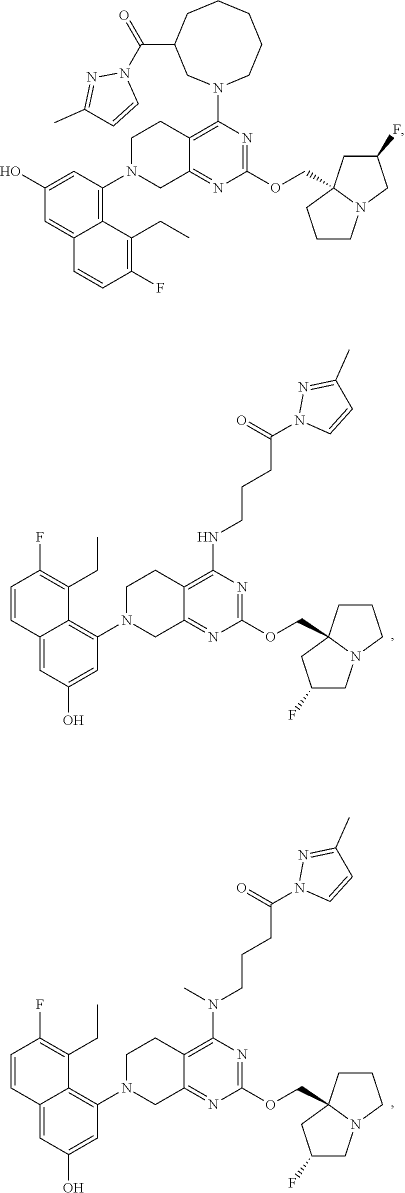 [SMILES: CCc1c(F)ccc2cc(O)cc(N3CCc4c(nc(OC[C@@]56CCCN5C[C@H](F)C6)nc4N4CCCCCC(C(=O)n5ccc(C)n5)C4)C3)c12 ; CCc1c(F)ccc2cc(O)cc(N3CCc4c(nc(OC[C@@]56CCCN5C[C@H](F)C6)nc4NCCCC(=O)n4ccc(C)n4)C3)c12 ; CCc1c(F)ccc2cc(O)cc(N3CCc4c(nc(OC[C@@]56CCCN5C[C@H](F)C6)nc4N(C)CCCC(=O)n4ccc(C)n4)C3)c12]

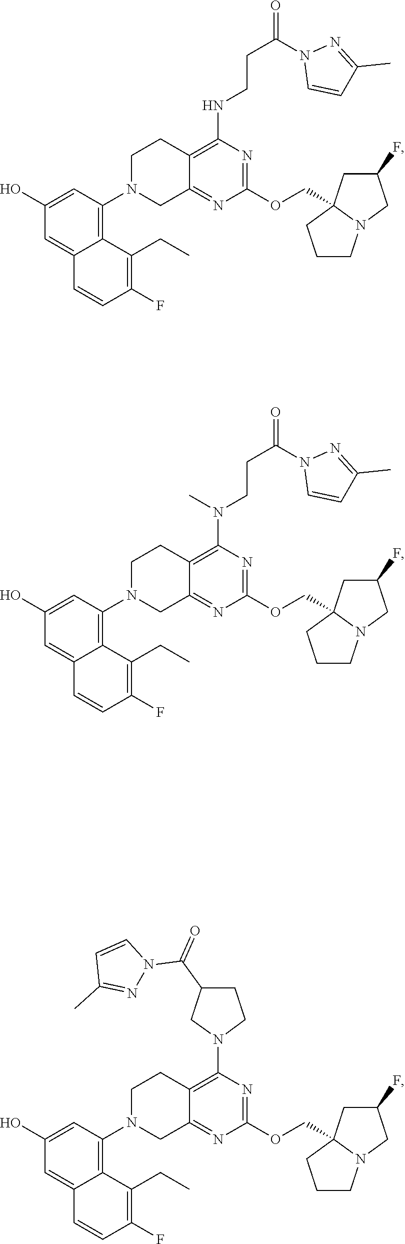 [SMILES: CCc1c(F)ccc2cc(O)cc(N3CCc4c(nc(OC[C@@]56CCCN5C[C@H](F)C6)nc4NCCC(=O)n4ccc(C)n4)C3)c12 ; CCc1c(F)ccc2cc(O)cc(N3CCc4c(nc(OC[C@@]56CCCN5C[C@H](F)C6)nc4N(C)CCC(=O)n4ccc(C)n4)C3)c12 ; CCc1c(F)ccc2cc(O)cc(N3CCc4c(nc(OC[C@@]56CCCN5C[C@H](F)C6)nc4N4CCC(C(=O)n5ccc(C)n5)C4)C3)c12]

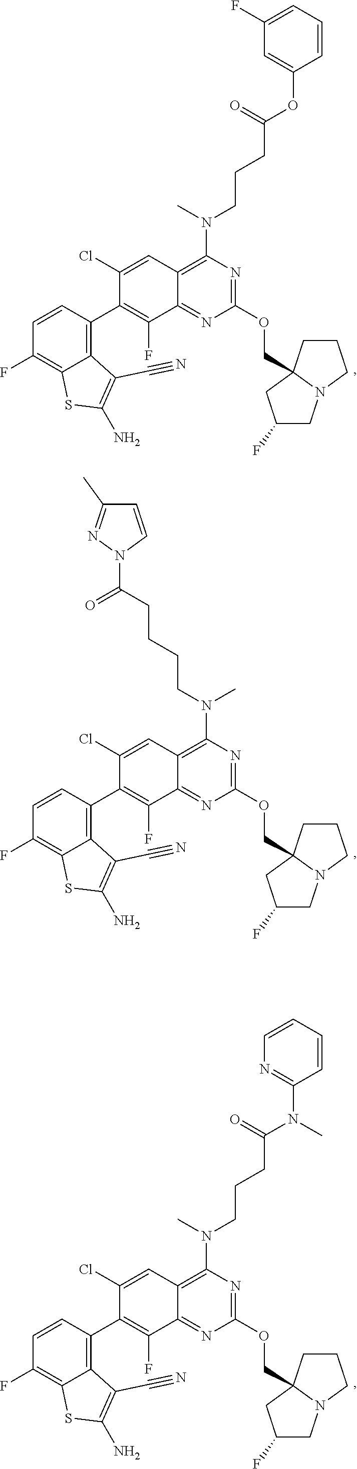 [SMILES: CN(CCCC(=O)Oc1cccc(F)c1)c1nc(OC[C@@]23CCCN2C[C@H](F)C3)nc2c(F)c(-c3ccc(F)c4sc(N)c(C#N)c34)c(Cl)cc12 ; Cc1ccn(C(=O)CCCCN(C)c2nc(OC[C@@]34CCCN3C[C@H](F)C4)nc3c(F)c(-c4ccc(F)c5sc(N)c(C#N)c45)c(Cl)cc23)n1 ; CN(CCCC(=O)N(C)c1ccccn1)c1nc(OC[C@@]23CCCN2C[C@H](F)C3)nc2c(F)c(-c3ccc(F)c4sc(N)c(C#N)c34)c(Cl)cc12]

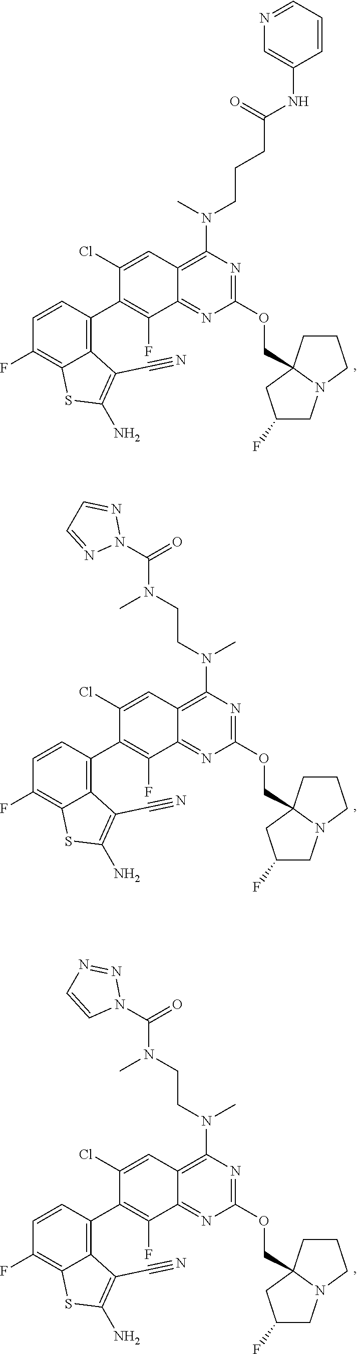 [SMILES: CN(CCCC(=O)Nc1cccnc1)c1nc(OC[C@@]23CCCN2C[C@H](F)C3)nc2c(F)c(-c3ccc(F)c4sc(N)c(C#N)c34)c(Cl)cc12 ; CN(CCN(C)c1nc(OC[C@@]23CCCN2C[C@H](F)C3)nc2c(F)c(-c3ccc(F)c4sc(N)c(C#N)c34)c(Cl)cc12)C(=O)n1nccn1 ; CN(CCN(C)c1nc(OC[C@@]23CCCN2C[C@H](F)C3)nc2c(F)c(-c3ccc(F)c4sc(N)c(C#N)c34)c(Cl)cc12)C(=O)n1ccnn1]

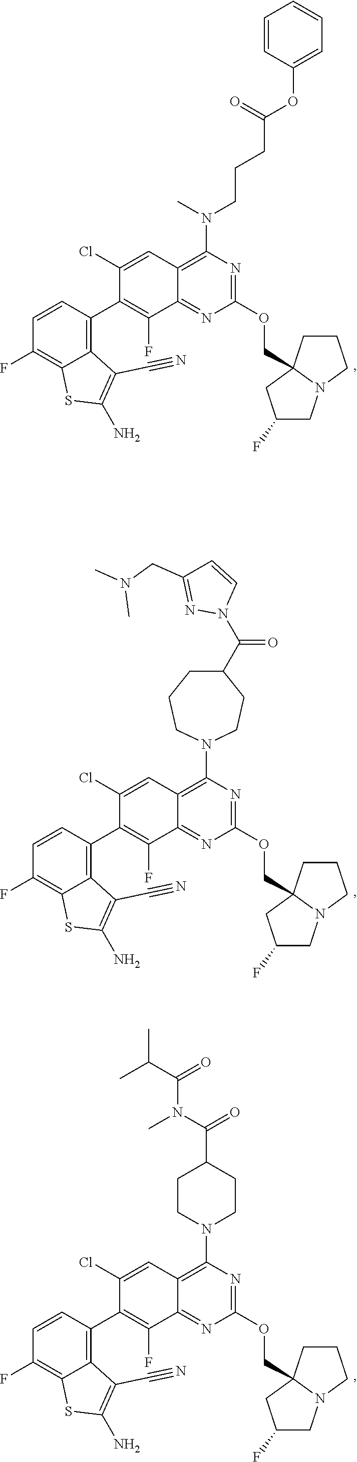 [SMILES: CN(CCCC(=O)Oc1ccccc1)c1nc(OC[C@@]23CCCN2C[C@H](F)C3)nc2c(F)c(-c3ccc(F)c4sc(N)c(C#N)c34)c(Cl)cc12 ; CN(C)Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; CC(C)C(=O)N(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1]

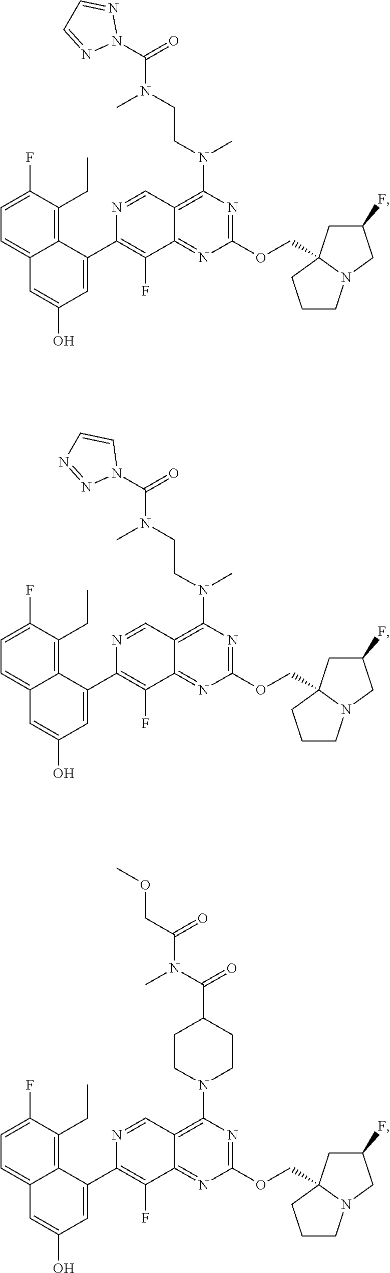 [SMILES: CCc1c(F)ccc2cc(O)cc(-c3ncc4c(N(C)CCN(C)C(=O)n5nccn5)nc(OC[C@@]56CCCN5C[C@H](F)C6)nc4c3F)c12 ; CCc1c(F)ccc2cc(O)cc(-c3ncc4c(N(C)CCN(C)C(=O)n5ccnn5)nc(OC[C@@]56CCCN5C[C@H](F)C6)nc4c3F)c12 ; CCc1c(F)ccc2cc(O)cc(-c3ncc4c(N5CCC(C(=O)N(C)C(=O)COC)CC5)nc(OC[C@@]56CCCN5C[C@H](F)C6)nc4c3F)c12]

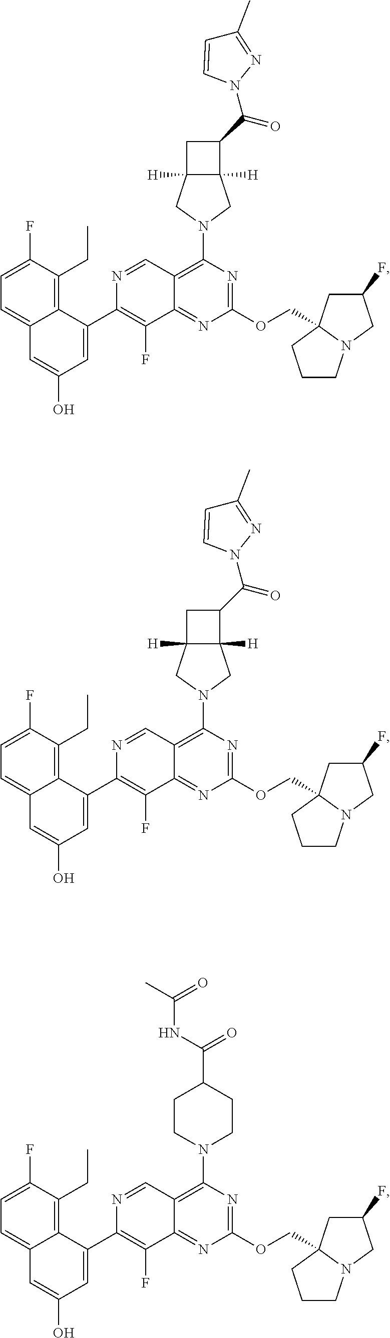 [SMILES: CCc1c(F)ccc2cc(O)cc(-c3ncc4c(N5C[C@H]6C[C@@H](C(=O)n7ccc(C)n7)[C@H]6C5)nc(OC[C@@]56CCCN5C[C@H](F)C6)nc4c3F)c12 ; CCc1c(F)ccc2cc(O)cc(-c3ncc4c(N5C[C@@H]6CC(C(=O)n7ccc(C)n7)[C@@H]6C5)nc(OC[C@@]56CCCN5C[C@H](F)C6)nc4c3F)c12 ; CCc1c(F)ccc2cc(O)cc(-c3ncc4c(N5CCC(C(=O)NC(C)=O)CC5)nc(OC[C@@]56CCCN5C[C@H](F)C6)nc4c3F)c12]

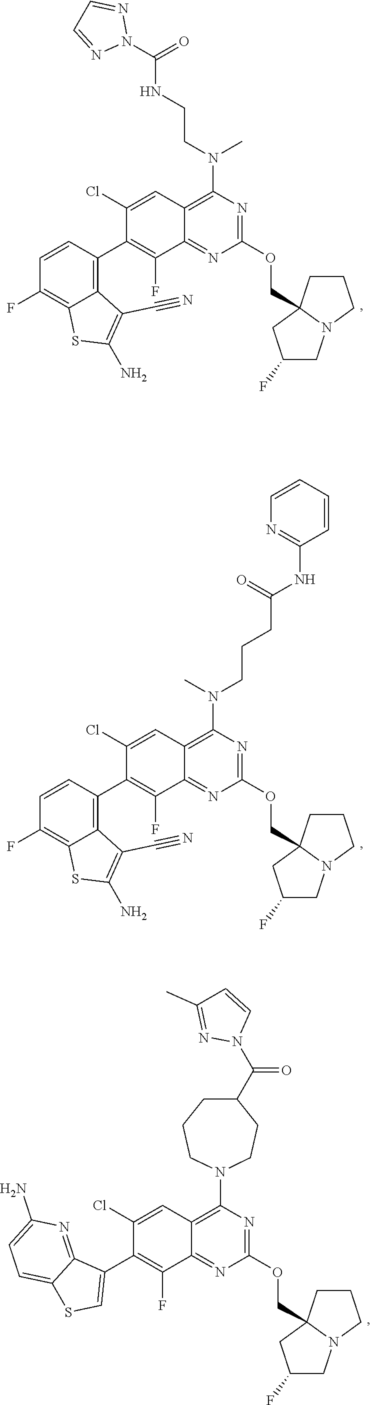 [SMILES: CN(CCNC(=O)n1nccn1)c1nc(OC[C@@]23CCCN2C[C@H](F)C3)nc2c(F)c(-c3ccc(F)c4sc(N)c(C#N)c34)c(Cl)cc12 ; CN(CCCC(=O)Nc1ccccn1)c1nc(OC[C@@]23CCCN2C[C@H](F)C3)nc2c(F)c(-c3ccc(F)c4sc(N)c(C#N)c34)c(Cl)cc12 ; Cc1ccn(C(=O)C2CCCN(c3nc(OC[C@@]45CCCN4C[C@H](F)C5)nc4c(F)c(-c5csc6ccc(N)nc56)c(Cl)cc34)CC2)n1]

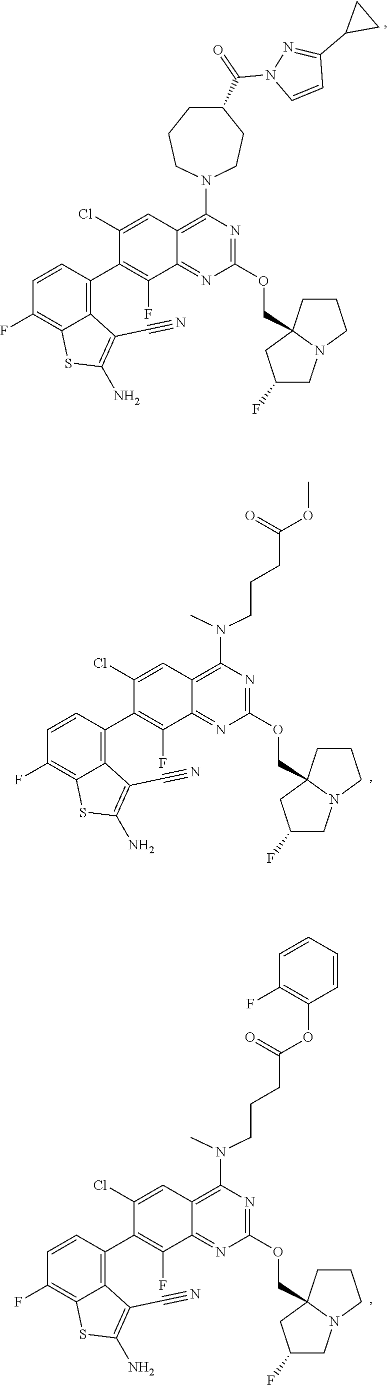 [SMILES: N#Cc1c(N)sc2c(F)ccc(-c3c(Cl)cc4c(N5CCC[C@H](C(=O)n6ccc(C7CC7)n6)CC5)nc(OC[C@@]56CCCN5C[C@H](F)C6)nc4c3F)c12 ; COC(=O)CCCN(C)c1nc(OC[C@@]23CCCN2C[C@H](F)C3)nc2c(F)c(-c3ccc(F)c4sc(N)c(C#N)c34)c(Cl)cc12 ; CN(CCCC(=O)Oc1ccccc1F)c1nc(OC[C@@]23CCCN2C[C@H](F)C3)nc2c(F)c(-c3ccc(F)c4sc(N)c(C#N)c34)c(Cl)cc12]

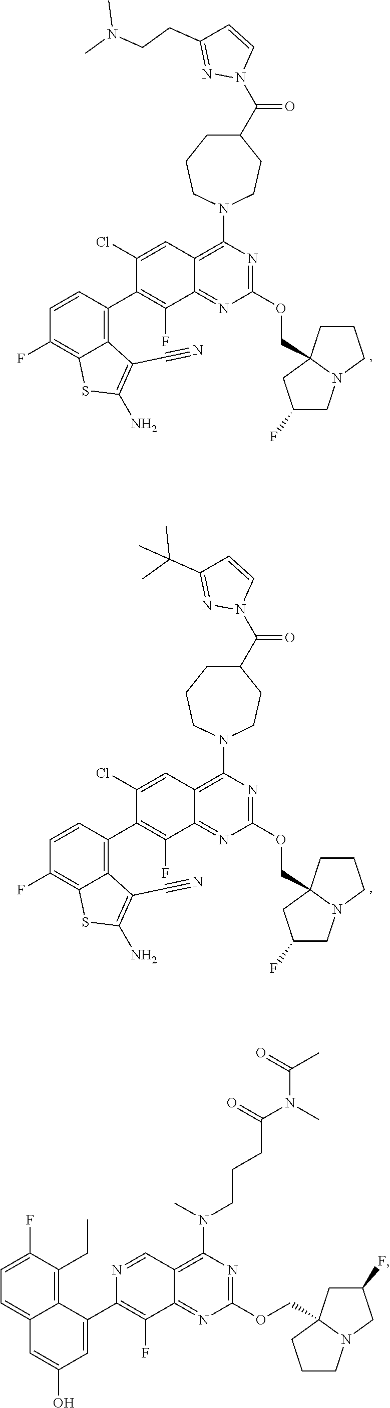 [SMILES: CN(C)CCc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; CC(C)(C)c1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; CCc1c(F)ccc2cc(O)cc(-c3ncc4c(N(C)CCCC(=O)N(C)C(C)=O)nc(OC[C@@]56CCCN5C[C@H](F)C6)nc4c3F)c12]

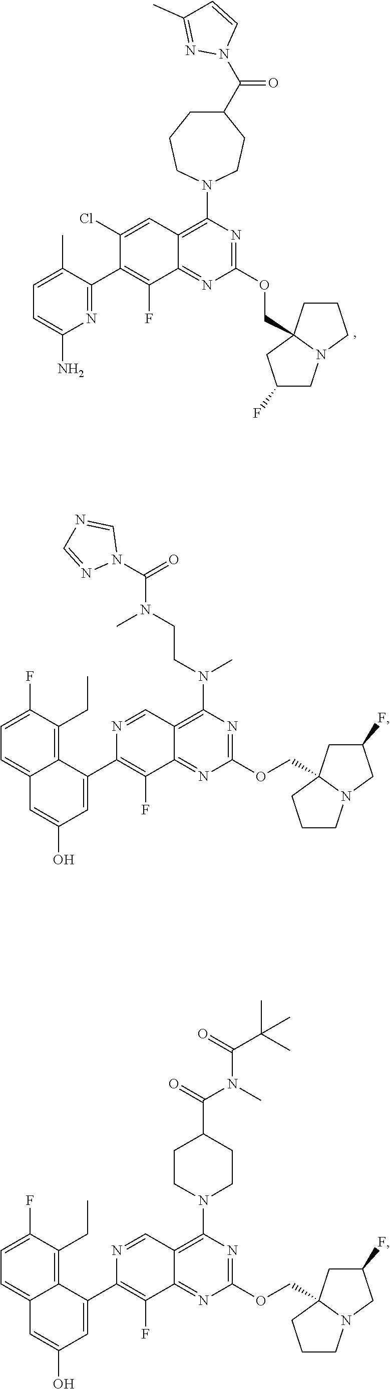 [SMILES: Cc1ccn(C(=O)C2CCCN(c3nc(OC[C@@]45CCCN4C[C@H](F)C5)nc4c(F)c(-c5nc(N)ccc5C)c(Cl)cc34)CC2)n1 ; CCc1c(F)ccc2cc(O)cc(-c3ncc4c(N(C)CCN(C)C(=O)n5cncn5)nc(OC[C@@]56CCCN5C[C@H](F)C6)nc4c3F)c12 ; CCc1c(F)ccc2cc(O)cc(-c3ncc4c(N5CCC(C(=O)N(C)C(=O)C(C)(C)C)CC5)nc(OC[C@@]56CCCN5C[C@H](F)C6)nc4c3F)c12]

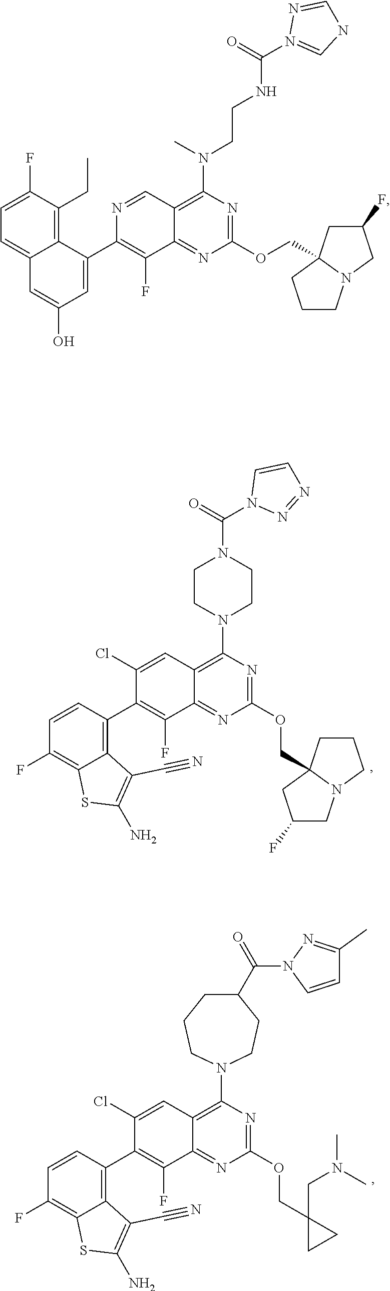

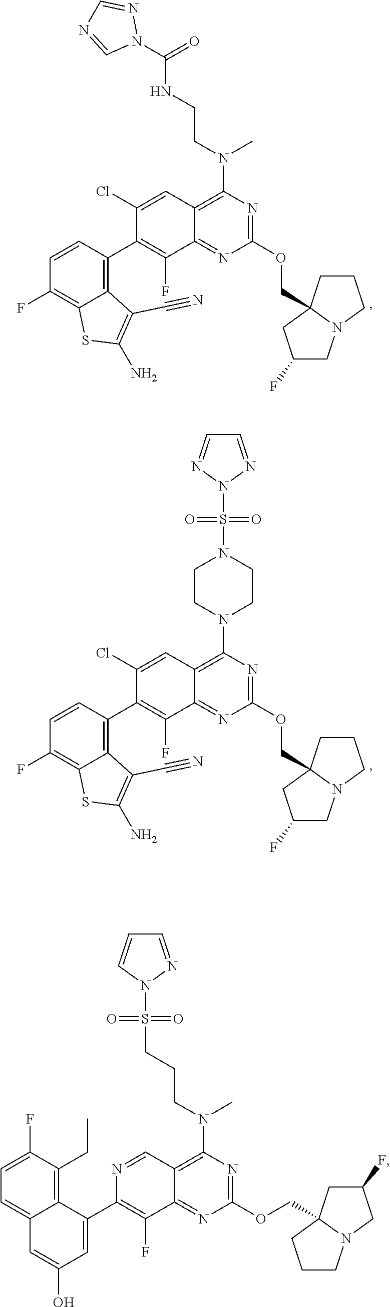 [SMILES: CN(CCNC(=O)n1cncn1)c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3c(Cl)cc4c(N5CCN(S(=O)(=O)n6nccn6)CC5)nc(OC[C@@]56CCCN5C[C@H](F)C6)nc4c3F)c12 ; CCc1c(F)ccc2cc(O)cc(-c3ncc4c(N(C)CCCS(=O)(=O)n5cccn5)nc(OC[C@@]56CCCN5C[C@H](F)C6)nc4c3F)c12]

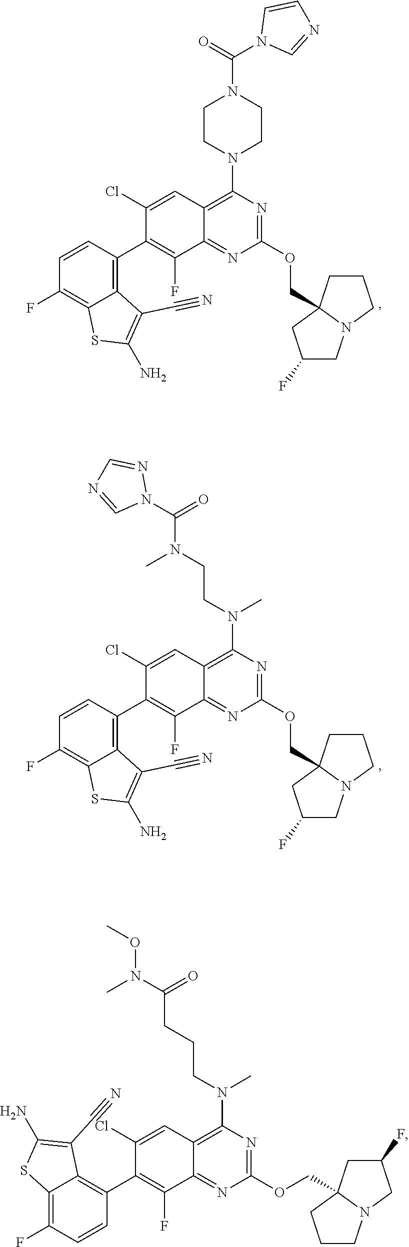 [SMILES: N#Cc1c(N)sc2c(F)ccc(-c3c(Cl)cc4c(N5CCN(C(=O)n6ccnc6)CC5)nc(OC[C@@]56CCCN5C[C@H](F)C6)nc4c3F)c12 ; CN(CCN(C)c1nc(OC[C@@]23CCCN2C[C@H](F)C3)nc2c(F)c(-c3ccc(F)c4sc(N)c(C#N)c34)c(Cl)cc12)C(=O)n1cncn1 ; CON(C)C(=O)CCCN(C)c1nc(OC[C@@]23CCCN2C[C@H](F)C3)nc2c(F)c(-c3ccc(F)c4sc(N)c(C#N)c34)c(Cl)cc12]

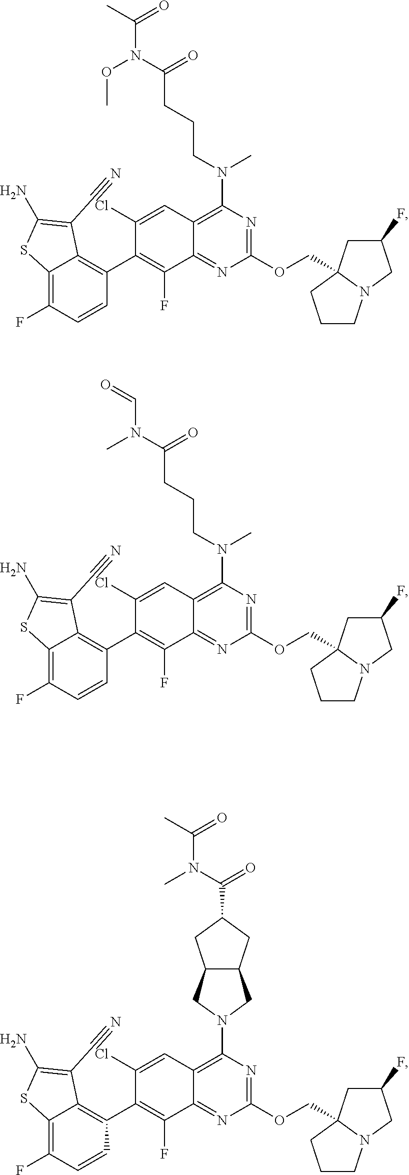

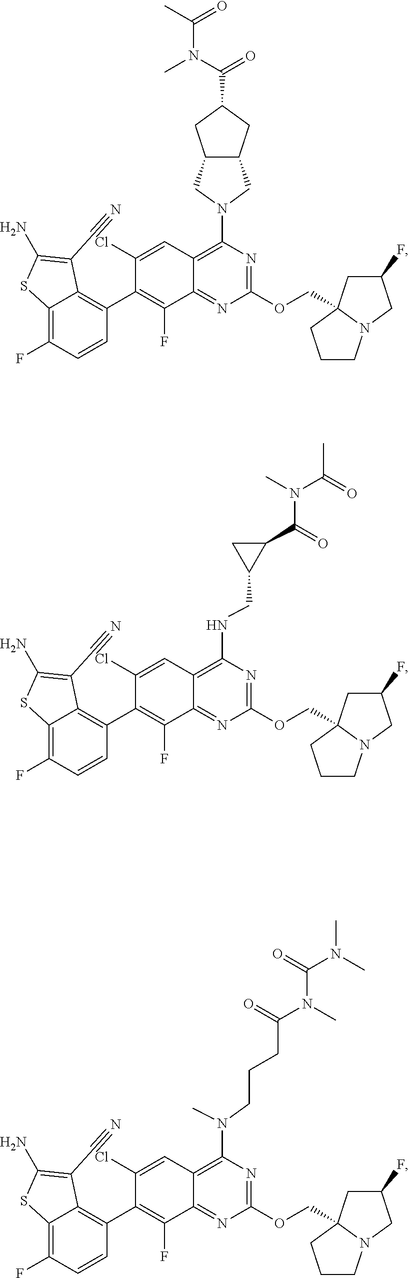 [SMILES: CC(=O)N(C)C(=O)[C@@H]1C[C@@H]2CN(c3nc(OC[C@@]45CCCN4C[C@H](F)C5)nc4c(F)c(-c5ccc(F)c6sc(N)c(C#N)c56)c(Cl)cc34)C[C@@H]2C1 ; CC(=O)N(C)C(=O)[C@@H]1C[C@H]1CNc1nc(OC[C@@]23CCCN2C[C@H](F)C3)nc2c(F)c(-c3ccc(F)c4sc(N)c(C#N)c34)c(Cl)cc12 ; CN(C)C(=O)N(C)C(=O)CCCN(C)c1nc(OC[C@@]23CCCN2C[C@H](F)C3)nc2c(F)c(-c3ccc(F)c4sc(N)c(C#N)c34)c(Cl)cc12]

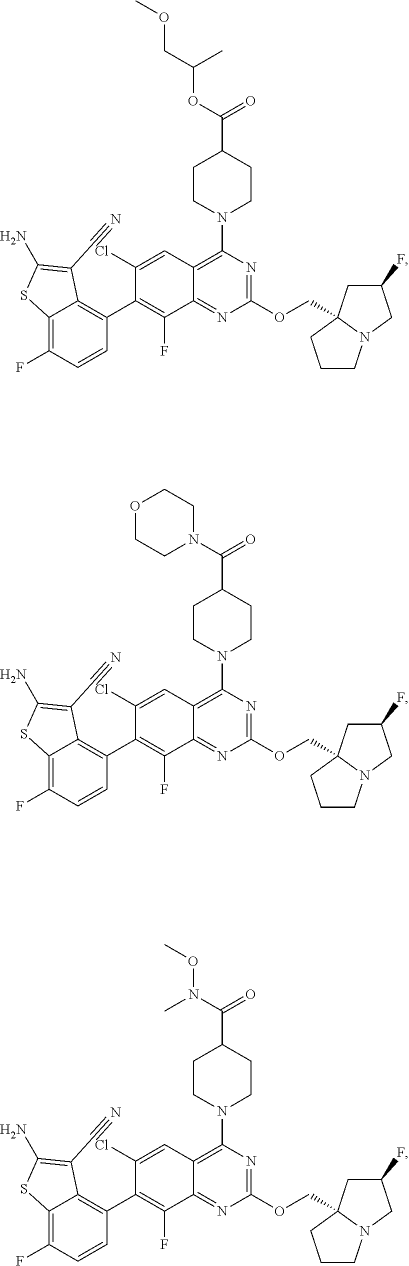 [SMILES: COCC(C)OC(=O)C1CCN(c2nc(OC[C@@]34CCCN3C[C@H](F)C4)nc3c(F)c(-c4ccc(F)c5sc(N)c(C#N)c45)c(Cl)cc23)CC1 ; N#Cc1c(N)sc2c(F)ccc(-c3c(Cl)cc4c(N5CCC(C(=O)N6CCOCC6)CC5)nc(OC[C@@]56CCCN5C[C@H](F)C6)nc4c3F)c12 ; CON(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1]

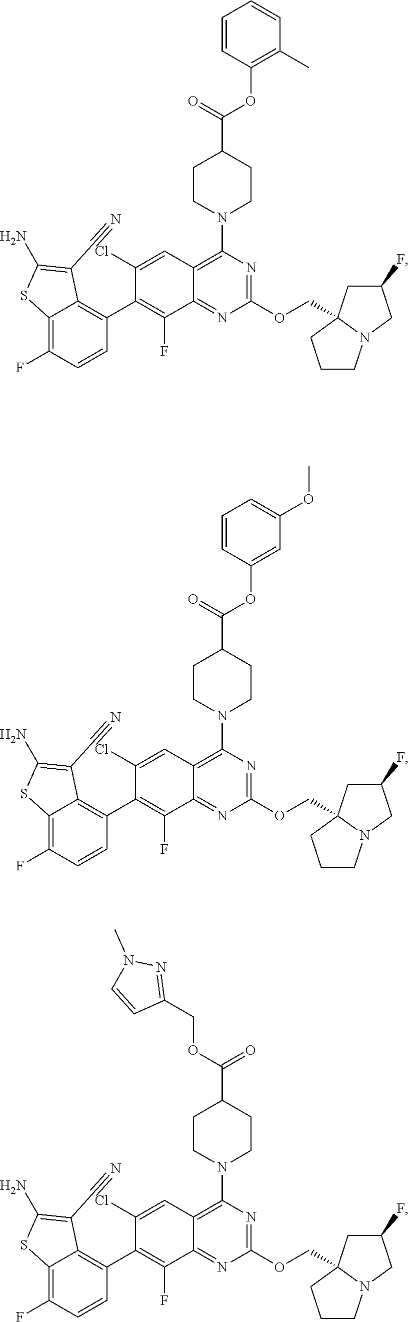 [SMILES: Cc1ccccc1OC(=O)C1CCN(c2nc(OC[C@@]34CCCN3C[C@H](F)C4)nc3c(F)c(-c4ccc(F)c5sc(N)c(C#N)c45)c(Cl)cc23)CC1 ; COc1cccc(OC(=O)C2CCN(c3nc(OC[C@@]45CCCN4C[C@H](F)C5)nc4c(F)c(-c5ccc(F)c6sc(N)c(C#N)c56)c(Cl)cc34)CC2)c1 ; Cn1ccc(COC(=O)C2CCN(c3nc(OC[C@@]45CCCN4C[C@H](F)C5)nc4c(F)c(-c5ccc(F)c6sc(N)c(C#N)c56)c(Cl)cc34)CC2)n1]

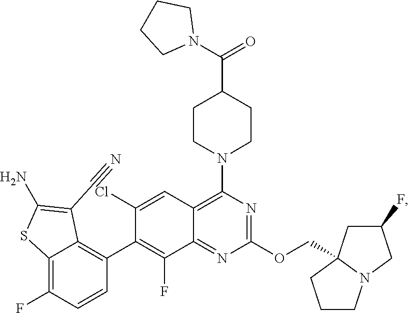 [SMILES: N#Cc1c(N)sc2c(F)ccc(-c3c(Cl)cc4c(N5CCC(C(=O)N6CCCC6)CC5)nc(OC[C@@]56CCCN5C[C@H](F)C6)nc4c3F)c12]

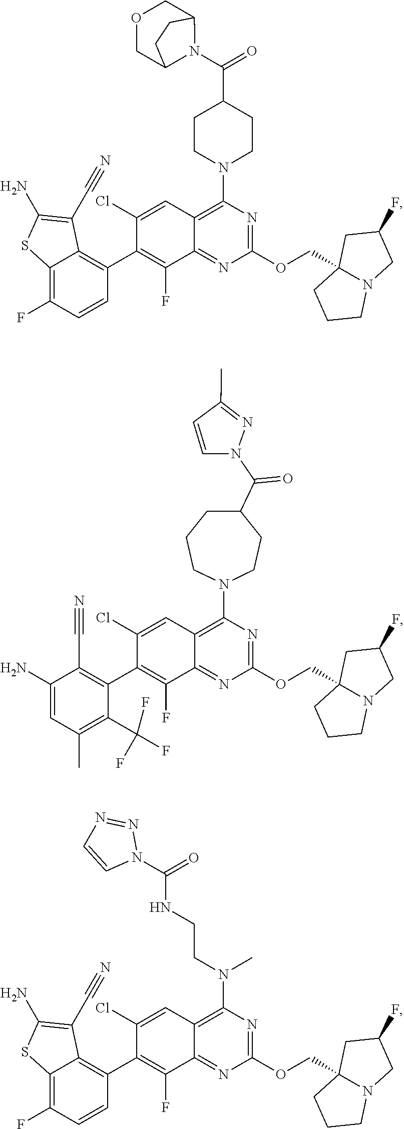 [SMILES: N#Cc1c(N)sc2c(F)ccc(-c3c(Cl)cc4c(N5CCC(C(=O)N6C7CCC6COC7)CC5)nc(OC[C@@]56CCCN5C[C@H](F)C6)nc4c3F)c12 ; Cc1ccn(C(=O)C2CCCN(c3nc(OC[C@@]45CCCN4C[C@H](F)C5)nc4c(F)c(-c5c(C#N)c(N)cc(C)c5C(F)(F)F)c(Cl)cc34)CC2)n1 ; CN(CCNC(=O)n1ccnn1)c1nc(OC[C@@]23CCCN2C[C@H](F)C3)nc2c(F)c(-c3ccc(F)c4sc(N)c(C#N)c34)c(Cl)cc12]

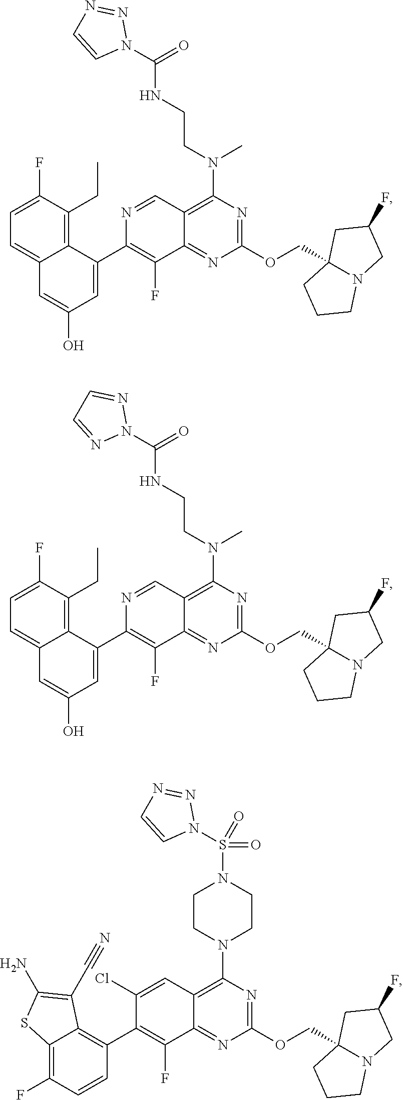 [SMILES: CCc1c(F)ccc2cc(O)cc(-c3ncc4c(N(C)CCNC(=O)n5ccnn5)nc(OC[C@@]56CCCN5C[C@H](F)C6)nc4c3F)c12 ; CCc1c(F)ccc2cc(O)cc(-c3ncc4c(N(C)CCNC(=O)n5nccn5)nc(OC[C@@]56CCCN5C[C@H](F)C6)nc4c3F)c12 ; N#Cc1c(N)sc2c(F)ccc(-c3c(Cl)cc4c(N5CCN(S(=O)(=O)n6ccnn6)CC5)nc(OC[C@@]56CCCN5C[C@H](F)C6)nc4c3F)c12]

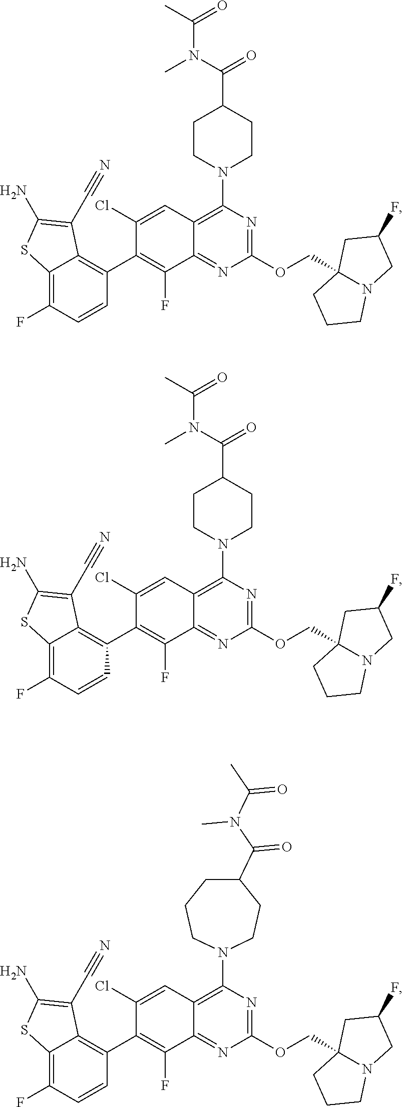

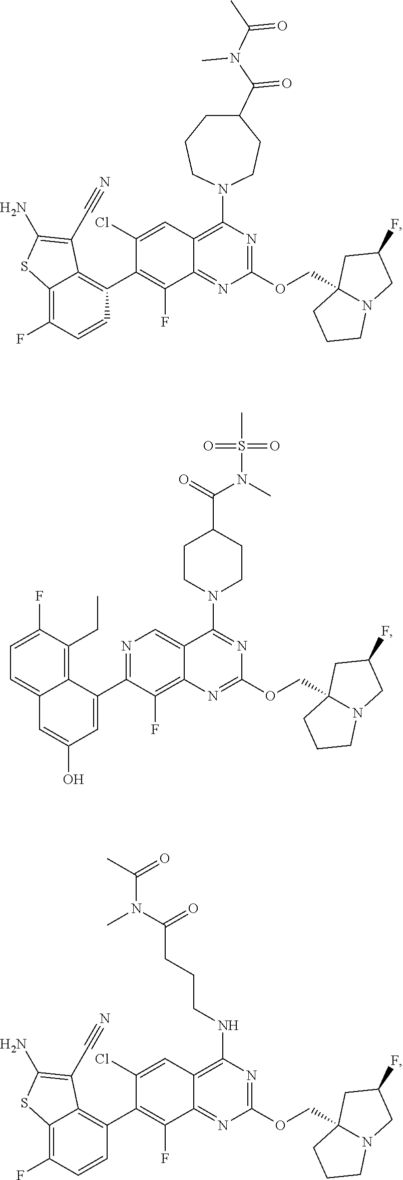

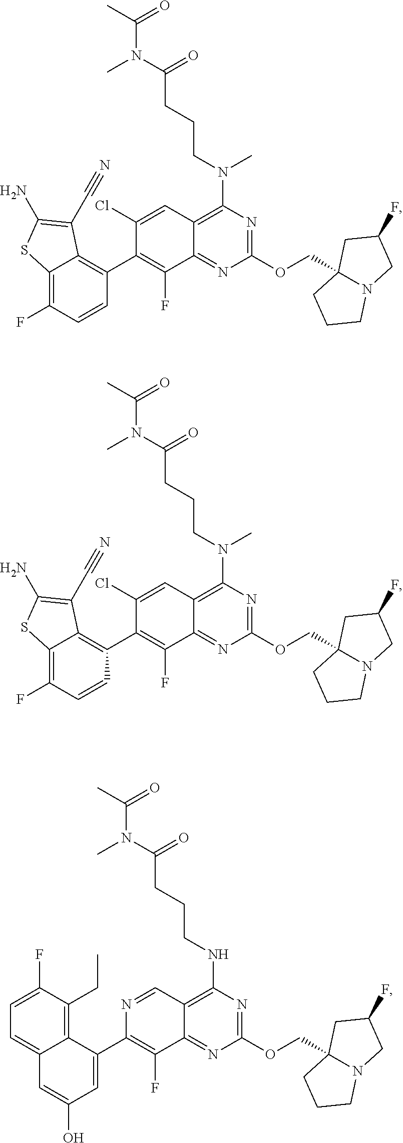

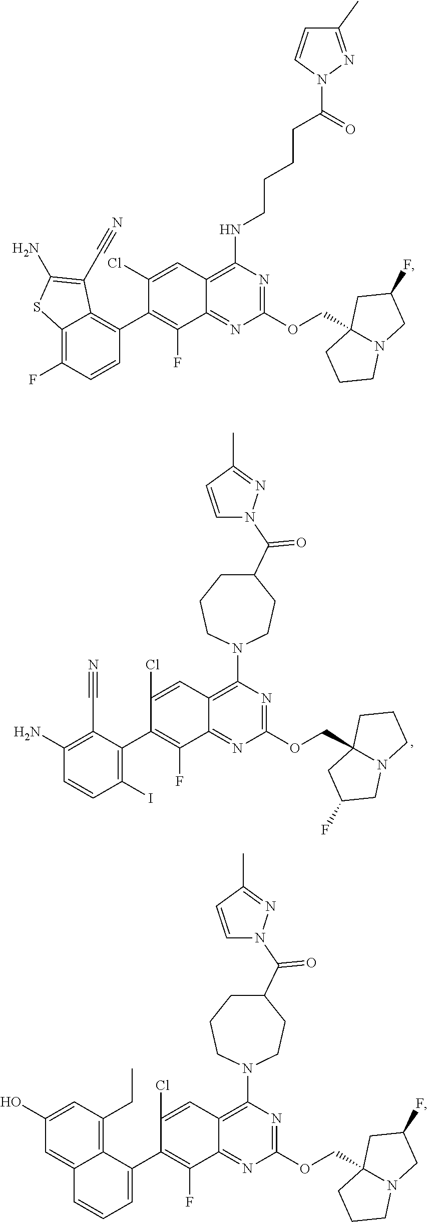 [SMILES: Cc1ccn(C(=O)CCCCNc2nc(OC[C@@]34CCCN3C[C@H](F)C4)nc3c(F)c(-c4ccc(F)c5sc(N)c(C#N)c45)c(Cl)cc23)n1 ; Cc1ccn(C(=O)C2CCCN(c3nc(OC[C@@]45CCCN4C[C@H](F)C5)nc4c(F)c(-c5c(I)ccc(N)c5C#N)c(Cl)cc34)CC2)n1 ; CCc1cc(O)cc2cccc(-c3c(Cl)cc4c(N5CCCC(C(=O)n6ccc(C)n6)CC5)nc(OC[C@@]56CCCN5C[C@H](F)C6)nc4c3F)c12]

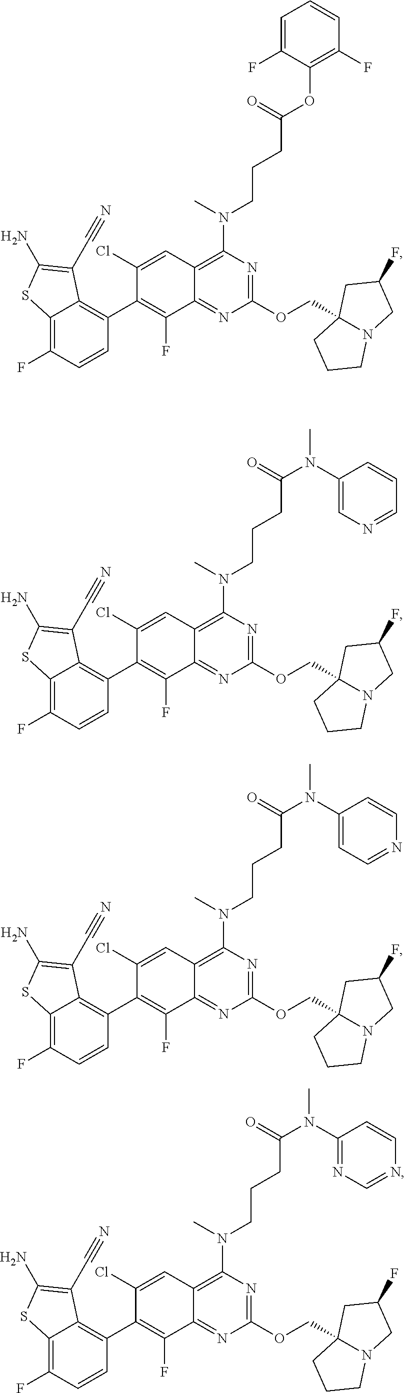

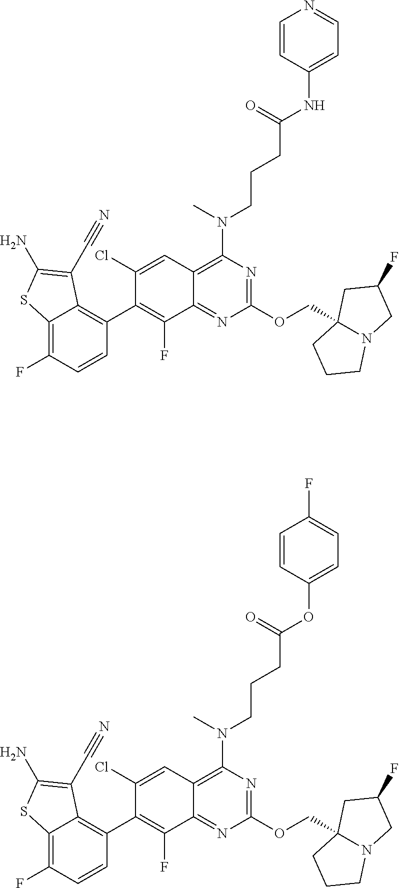 [SMILES: CN(CCCC(=O)Nc1ccncc1)c1nc(OC[C@@]23CCCN2C[C@H](F)C3)nc2c(F)c(-c3ccc(F)c4sc(N)c(C#N)c34)c(Cl)cc12 ; CN(CCCC(=O)Oc1ccc(F)cc1)c1nc(OC[C@@]23CCCN2C[C@H](F)C3)nc2c(F)c(-c3ccc(F)c4sc(N)c(C#N)c34)c(Cl)cc12]

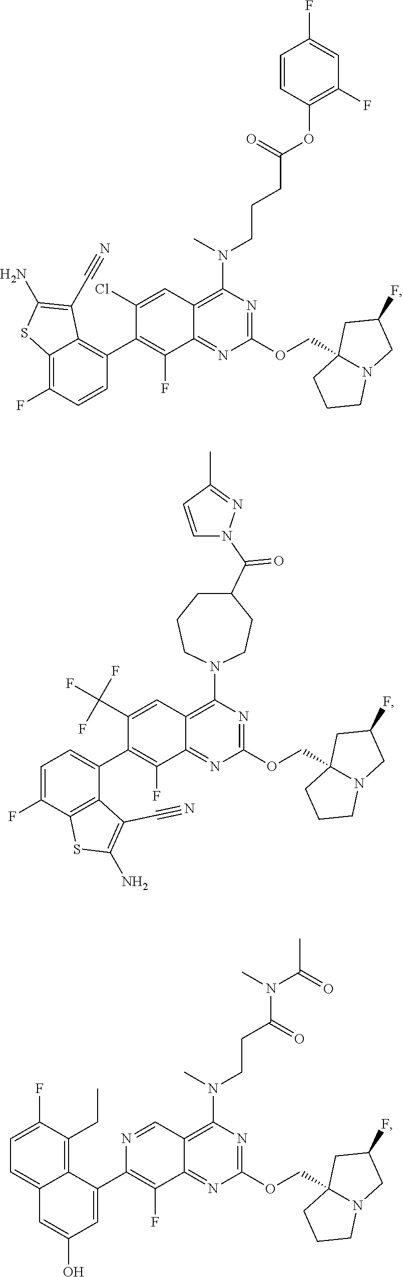 [SMILES: CN(CCCC(=O)Oc1ccc(F)cc1F)c1nc(OC[C@@]23CCCN2C[C@H](F)C3)nc2c(F)c(-c3ccc(F)c4sc(N)c(C#N)c34)c(Cl)cc12 ; Cc1ccn(C(=O)C2CCCN(c3nc(OC[C@@]45CCCN4C[C@H](F)C5)nc4c(F)c(-c5ccc(F)c6sc(N)c(C#N)c56)c(C(F)(F)F)cc34)CC2)n1 ; CCc1c(F)ccc2cc(O)cc(-c3ncc4c(N(C)CCC(=O)N(C)C(C)=O)nc(OC[C@@]56CCCN5C[C@H](F)C6)nc4c3F)c12]

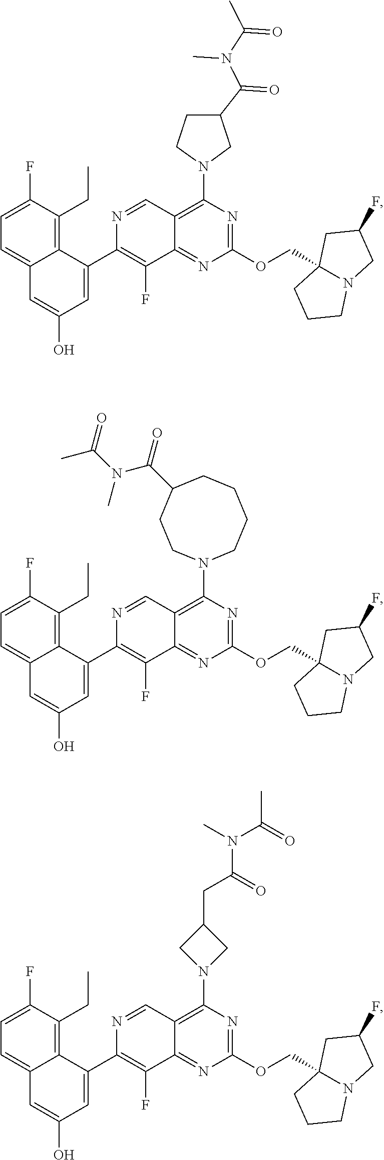 [SMILES: CCc1c(F)ccc2cc(O)cc(-c3ncc4c(N5CCC(C(=O)N(C)C(C)=O)C5)nc(OC[C@@]56CCCN5C[C@H](F)C6)nc4c3F)c12 ; CCc1c(F)ccc2cc(O)cc(-c3ncc4c(N5CCCCC(C(=O)N(C)C(C)=O)CC5)nc(OC[C@@]56CCCN5C[C@H](F)C6)nc4c3F)c12 ; CCc1c(F)ccc2cc(O)cc(-c3ncc4c(N5CC(CC(=O)N(C)C(C)=O)C5)nc(OC[C@@]56CCCN5C[C@H](F)C6)nc4c3F)c12]

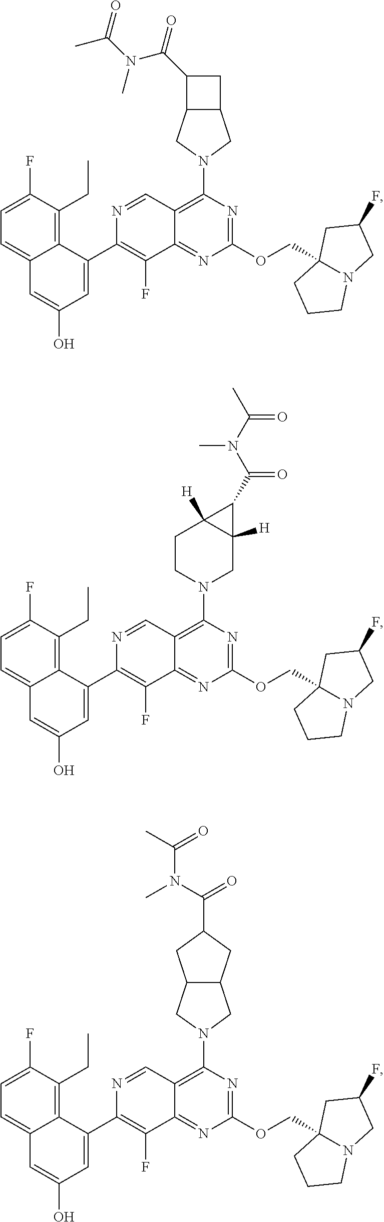 [SMILES: CCc1c(F)ccc2cc(O)cc(-c3ncc4c(N5CC6CC(C(=O)N(C)C(C)=O)C6C5)nc(OC[C@@]56CCCN5C[C@H](F)C6)nc4c3F)c12 ; CCc1c(F)ccc2cc(O)cc(-c3ncc4c(N5CC[C@H]6[C@@H](C5)[C@H]6C(=O)N(C)C(C)=O)nc(OC[C@@]56CCCN5C[C@H](F)C6)nc4c3F)c12 ; CCc1c(F)ccc2cc(O)cc(-c3ncc4c(N5CC6CC(C(=O)N(C)C(C)=O)CC6C5)nc(OC[C@@]56CCCN5C[C@H](F)C6)nc4c3F)c12]

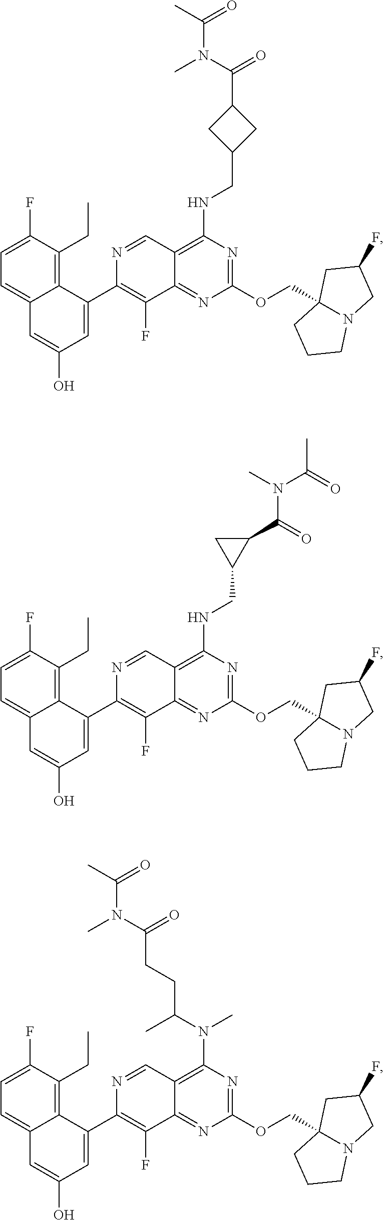 [SMILES: CCc1c(F)ccc2cc(O)cc(-c3ncc4c(NCC5CC(C(=O)N(C)C(C)=O)C5)nc(OC[C@@]56CCCN5C[C@H](F)C6)nc4c3F)c12 ; CCc1c(F)ccc2cc(O)cc(-c3ncc4c(NC[C@@H]5C[C@H]5C(=O)N(C)C(C)=O)nc(OC[C@@]56CCCN5C[C@H](F)C6)nc4c3F)c12 ; CCc1c(F)ccc2cc(O)cc(-c3ncc4c(N(C)C(C)CCC(=O)N(C)C(C)=O)nc(OC[C@@]56CCCN5C[C@H](F)C6)nc4c3F)c12]

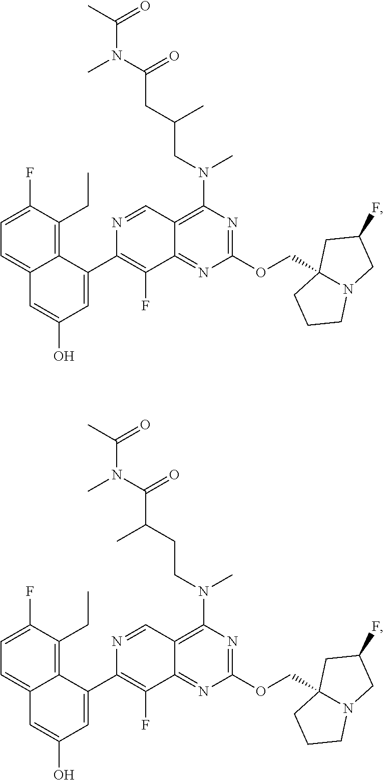 [SMILES: CCc1c(F)ccc2cc(O)cc(-c3ncc4c(N(C)CC(C)CC(=O)N(C)C(C)=O)nc(OC[C@@]56CCCN5C[C@H](F)C6)nc4c3F)c12 ; CCc1c(F)ccc2cc(O)cc(-c3ncc4c(N(C)CCC(C)C(=O)N(C)C(C)=O)nc(OC[C@@]56CCCN5C[C@H](F)C6)nc4c3F)c12]

[chemical structure]

[chemical structure]

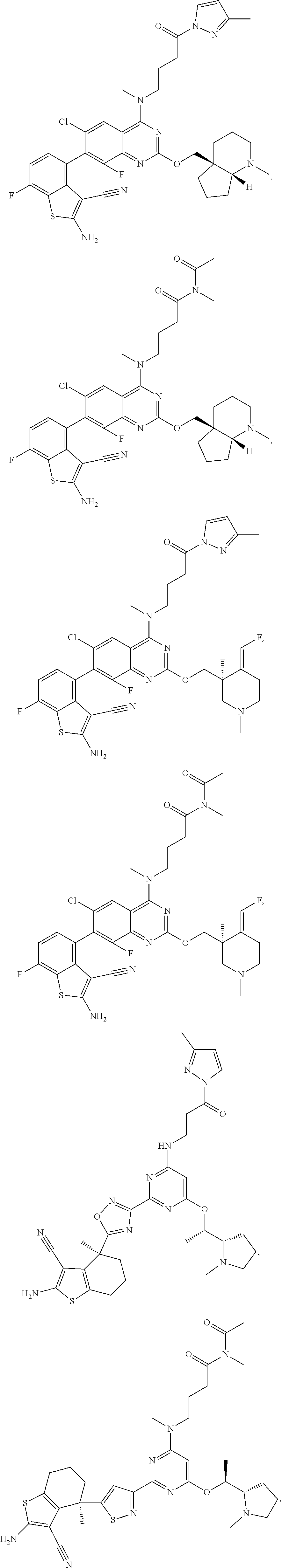

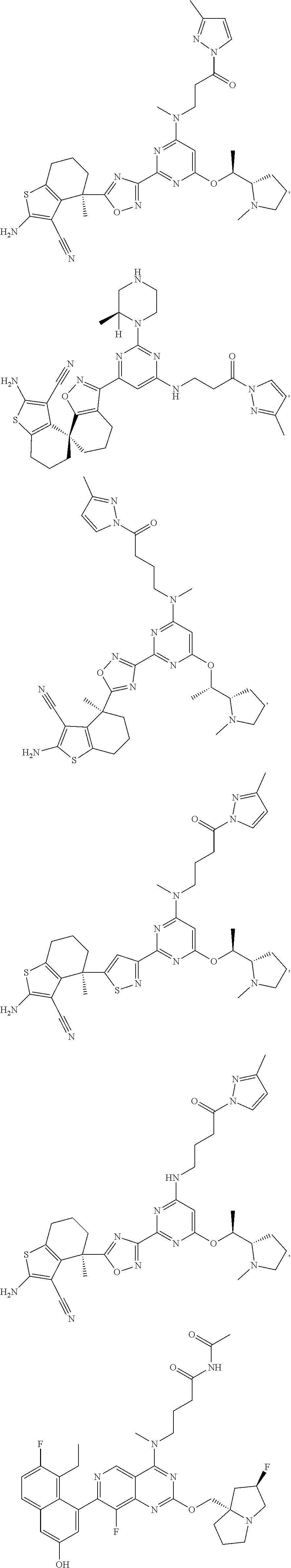

and pharmaceutically acceptable salts thereof.

In one embodiment, the compounds of Formula (Ia) or (Ib), Formula (IA) and/or Formula (IB), include bis-hydrochloride, formic acid, bis-formic acid, tris-hydrochloride, trifluoroacetic acid, bis-trifluoroacetic acid, and tris-trifluoracetic acid salts of the above compounds. The compounds of Formula (Ia) or (Ib), or pharmaceutically acceptable salt thereof may be formulated into pharmaceutical compositions.

### Pharmaceutical Compositions

In another aspect, the invention provides pharmaceutical compositions comprising a KRas G12S and/or KRas G12C inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In one embodiment, administration may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 100 mg/kg, for example 0.1 to 50 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods of use described herein.

### Methods of Use

In yet another aspect, the invention provides for methods for inhibiting KRas G12S and/or KRas G12C activity in a cell, comprising contacting the cell in which inhibition of KRas G12S activity is desired with an effective amount of a compound of Formula (Ia) or (Ib), pharmaceutically acceptable salts thereof, or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” a KRas G12S with a compound provided herein includes the administration of a compound provided herein to an individual or patient, such as a human, having KRas G12S, as well as, for example, introducing a compound provided herein into a sample containing a cellular or purified preparation containing the KRas G12S. For example, “contacting” a KRas G12C with a compound provided herein includes the administration of a compound provided herein to an individual or patient, such as a human, having KRas G12C, as well as, for example, introducing a compound provided herein into a sample containing a cellular or purified preparation containing the KRas G12C.

In one embodiment, a cell in which inhibition of KRas G12S and/or KRas G12C activity is desired is contacted with an effective amount of a compound of Formula (Ia) or (Ib), or pharmaceutically acceptable salt thereof to negatively modulate the activity of KRas G12S and/or KRas G12C.

By negatively modulating the activity of KRas G12S and/or KRas G12C, the methods described herein are designed to inhibit undesired cellular proliferation resulting from enhanced KRas G12S and/or KRas G12C activity within the cell. The cells may be contacted in a single dose or multiple doses in accordance with a particular treatment regimen to affect the desired negative modulation of KRas G12S and/or KRas G12C. The ability of compounds to bind KRas G12S and/or KRas G12C may be monitored in vitro using well known methods, including those described in Examples A and B below. In addition, the inhibitory activity of exemplary compounds in cells may be monitored, for example, by measuring the inhibition of KRas G12S and/or KRas G12C activity of the amount of phosphorylated ERK, for example using the method described in Example C below.

In another aspect, methods of treating cancer in a patient in need thereof, comprising administering to said patient a therapeutically effective amount of a compound of Formula (Ia) or (Ib), or a pharmaceutically acceptable salt thereof, or a pharmaceutical composition comprising the compound or pharmaceutically acceptable salt thereof are provided.

The compositions and methods provided herein may be used for the treatment of a KRas G12S-associated cancer and/or KRas G12C-associated cancer in a patient in need thereof, comprising administering to said patient a therapeutically effective amount of a compound of Formula (Ia) or (Ib), a pharmaceutically acceptable salt thereof, or a pharmaceutical composition comprising the compound or pharmaceutically acceptable salt thereof are provided. In one embodiment, the KRas G12S-associated cancer is lung cancer. In one embodiment, the KRas G12C-associated cancer is lung cancer

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the invention include but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

In certain embodiments, the cancer is non-small cell lung cancer, small cell lung cancer, colorectal cancer, rectal cancer or pancreatic cancer. In certain embodiments, the cancer is non-small cell lung cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

Also provided herein is a compound of Formula (Ia) or (Ib), or a pharmaceutically acceptable salt thereof, or a pharmaceutical composition thereof as defined herein for use in therapy.

Also provided herein is a compound of Formula (Ia) or (Ib), or a pharmaceutically acceptable salt thereof, or a pharmaceutical composition thereof as defined herein for use in the treatment of cancer.

Also provided herein is a compound of Formula (Ia) or (Ib), or a pharmaceutically acceptable salt thereof, or a pharmaceutical composition thereof for use in the inhibition of KRas G12S and/or KRas G12C.

Also provided herein is a compound of Formula (Ia) or (Ib), or a pharmaceutically acceptable salt thereof, or a pharmaceutical composition thereof as defined herein, for use in the treatment of a KRas G12S-associated and/or a KRas G12C-associated disease or disorder.

Also provided herein is the use of a compound of Formula (Ia) or (Ib), or a pharmaceutically acceptable salt thereof, as defined herein in the manufacture of a medicament for the treatment of cancer.

Also provided herein is a use of a compound of Formula (Ia) or (Ib), or a pharmaceutically acceptable salt thereof, as defined herein in the manufacture of a medicament for the inhibition of activity of KRas G12S and/or KRas G12C.

Also provided herein is the use of a compound of Formula (Ia) or (Ib), or a pharmaceutically acceptable salt thereof, as defined herein, in the manufacture of a medicament for the treatment of a KRas G12S-associated and/or a KRas G12C-associated disease or disorder.

Also provided herein is a method for treating cancer in a patient in need thereof, the method comprising (a) determining that cancer is associated with a KRas G12S mutation or a KRas G12C mutation (e.g., a KRas G12S-associated cancer or a KRas G12C-associated cancer) (e.g., as determined using a regulatory agency-approved, e.g., FDA-approved, assay or kit); and (b) administering to the patient a therapeutically effective amount of a compound of Formula (Ia) or (Ib), or a pharmaceutically acceptable salt thereof, or a pharmaceutical composition thereof.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### EXAMPLES

The compounds of the present invention may be prepared from commercially available reagents using the synthetic methods and reaction schemes described herein, or using other reagents and conventional methods well known to those skilled in the art.

The following Examples are intended to illustrate further certain embodiments of the invention and are not intended to limit the scope of the invention.

### REAGENT ABBREVIATION

DCM Dichloromethane
DMAP 4-Dimethylaminopyridine
EDCI 1-Ethyl-3-(3-dimethylaminopropyl)carbodiimide
TFA Trifluoroacetic acid
DMF Dimethylformamide
HATU Hexafluorophosphate azabenzotriazole tetramethyl uranium (1-((dimethylamino)(dimethyliminio)methyl)-1H-[1,2,3]triazolo[4,5-b]pyridine 4-oxide hexafluorophosphate (V))
DMAc Dimethylacetamide
DIEA N,N-Diisopropylethylamine
PyBOP Benzotriazol-1-yloxytripyrrolidinophosphonium hexafluorophosphate
HOBt 1-Hydroxybenzotriazole
DMP Dess-Martin periodinane
TEA Triethylamine
CMPI 2-Chloro-1-methylpyridinium iodide
DCC N,N′-Dicyclohexylcarbodiimide

### Example 1

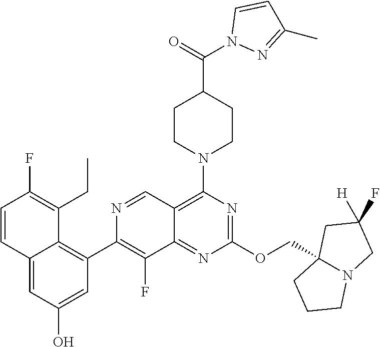 [SMILES: CCc1c(F)ccc2cc(O)cc(-c3ncc4c(N5CCC(C(=O)n6ccc(C)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-methyl-1H-pyrazol-1-yl)methanone

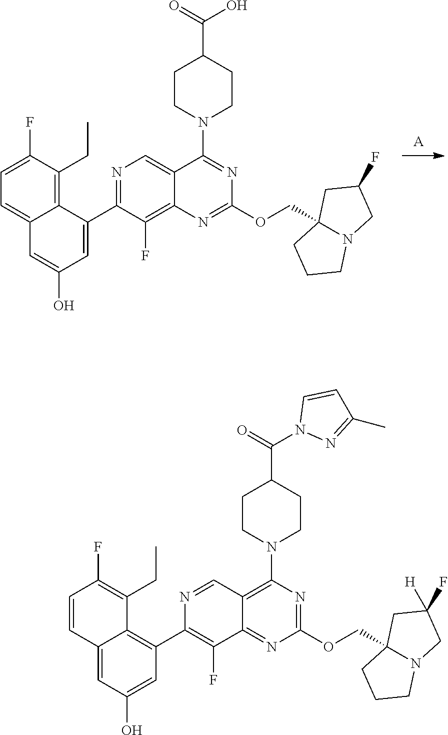

Step A. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (50 mg, 1.0 equiv) and 3-methyl-1H-pyrazole (10.0 mg, 1.5 equiv) in DCM (1 mL) were added DMAP (12.3 mg, 1.2 equiv) and EDCI (19.3 mg, 1.2 equiv). The reaction was stirred at 25 °C for 12 hours. The mixture was concentrated and purified by prep-HPLC [column: Phenomenex luna C18 150×25 mm×5 μm; A: water (FA), B: ACN, B %: 15%-45% B over 10 min] to afford the title compound (12.1 mg, 22% yield) as white solid; ¹H NMR (400 MHz, CHLOROFORM-d) δ=8.96 (s, 1H), 8.16 (d, J=2.8 Hz, 1H), 7.54 (dd, J=5.6, 8.8 Hz, 1H), 7.23-7.12 (m, 2H), 7.09-6.96 (m, 1H), 6.29 (d, J=2.8 Hz, 1H), 5.44-5.21 (m, 1H), 4.71-4.54 (m, 2H), 4.42-4.23 (m, 2H), 4.01 (dt, J=5.2, 10.4 Hz, 1H), 3.63-3.11 (m, 6H), 3.08-2.94 (m, 1H), 2.46 (br d, J=7.6 Hz, 1H), 2.36 (s, 3H), 2.24-1.99 (m, 10H), 0.83-0.74 (m, 3H); LCMS (ESI, M+1): m/z=686.4.

### Example 2

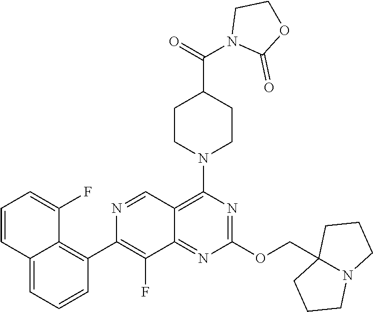 [SMILES: O=C1OCCN1C(=O)C1CCN(c2nc(OCC34CCCN3CCC4)nc3c(F)c(-c4cccc5cccc(F)c45)ncc23)CC1]

### 3-(1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carbonyl)oxazolidin-2-one

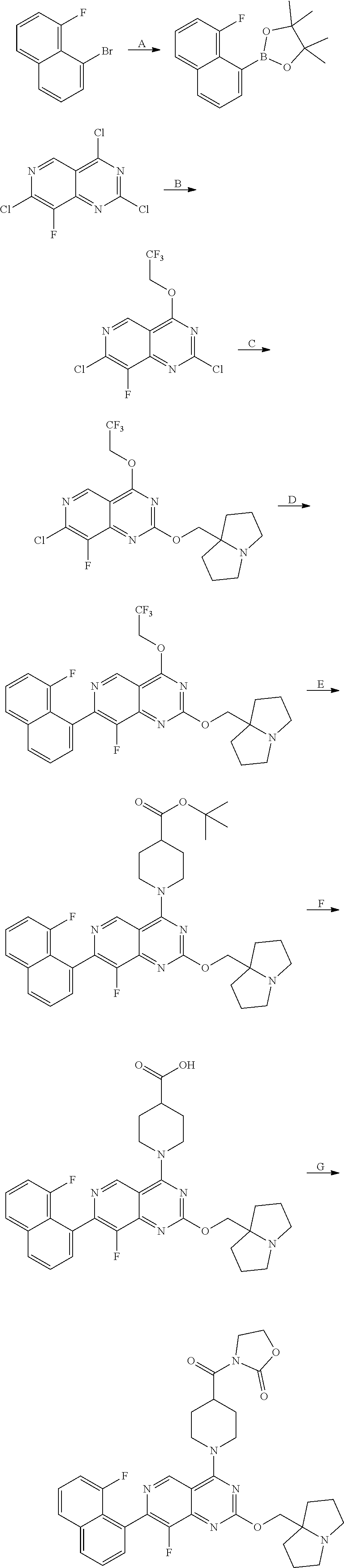

Step A. 2-(8-fluoronaphthalen-1-yl)-4,4,5,5-tetramethyl-1,3,2-dioxaborolane: To a solution of 1-bromo-8-fluoronaphthalene (9.00 g, 1.0 equiv) and 4,4,5,5-tetramethyl-2-(4,4,5,5-tetramethyl-1,3,2-dioxaborolan-2-yl)-1,3,2-dioxaborolane (15.2 g, 1.5 equiv) in dioxane (100 mL) were added potassium 3.0 equiv) and [1,1′-acetate (11.8 g. bis(diphenylphosphino)ferrocene]dichloropalladium(II) (2.93 g, 0.10 equiv). The reaction was degassed and purged with nitrogen 3 times. The reaction was stirred at 90 °C for 1 hour. The mixture was diluted with H₂O (100 mL) and extracted with dichloromethane (3×100 mL). The organic layer was dried over anhydrous sodium sulfate, concentrated and purified with column chromatography [SiO₂, Petroleum ether/Ethyl acetate=50/1 to 1/1] to afford the title compound (8.50 g, 78% yield) as a white solid; ¹H NMR (400 MHz, DMSO-d6) δ=8.04 (td, J=2.4, 8.0 Hz, 1H), 7.79 (d, J=8.0 Hz, 1H), 7.64-7.56 (m, 2H), 7.52 (dt, J=5.2, 8.0 Hz, 1H), 7.35 (dd, J=8.0, 12.0 Hz, 1H), 1.36 (s, 12H).

Step B. 2,7-dichloro-8-fluoro-4-(2,2,2-trifluoroethoxy)pyrido[4,3-d]pyrimidine: To a solution of 2,2,2-trifluoroethanol (4.36 g, 1.1 equiv) in THF (50 mL) was added t-BuONa (4.19 g, 1.1 equiv) in portions at 25 °C. The reaction was stirred at 25 °C for 1 hour. The mixture was added into a solution of 2,4,7-trichloro-8-fluoropyrido[4,3-d]pyrimidine (10.0 g, 1.0 equiv) in THF (100 mL) at −40 °C. The reaction was stirred at 0 °C for 1 hour. The mixture was diluted with H₂O (100 mL) and extracted with ethyl acetate (3×100 mL). The combined organic layers were dried over anhydrous sodium sulfate, concentrated, and purified by column chromatography [SiO₂, Petroleum ether/Ethyl acetate=30/1 to 1/1] to afford the title compound (7.10 g, 80% yield) as white solid; LCMS (ESI, M+1): m/z=315.0.

Step C: 7-chloro-8-fluoro-2-((tetrahydro-1H-pyrrolizin-7a(5H)-yl)methoxy)-4-(2,2,2-trifluoroethoxy)pyrido[4,3-d]pyrimidine: To a solution of 2,7-dichloro-8-fluoro-4-(2,2,2-trifluoroethoxy)pyrido[4,3-d]pyrimidine (7.00 g, 1.0 equiv) and 1,2,3,5,6,7-hexahydropyrrolizin-8-ylmethanol (3.13 g, 1.0 equiv) in THF (70 mL) was added Na₂CO₃ (7.04 g, 3.0 equiv). The reaction was stirred at 40 °C for 3 hours. The mixture was diluted with H₂O (100 mL) and extracted with dichloromethane (3×100 mL). The combined organic layers were dried over anhydrous sodium sulfate, concentrated and purified by column chromatography [SiO₂, Petroleum ether/Ethyl acetate=10/1 to 0/1] to afford the title compound (8.50 g, 83% yield) as white solid; LCMS (ESI, M+1): m/z=421.1.

step D: 8-fluoro-7-(8-fluoronaphthalen-1-yl)-2-((tetrahydro-1H-pyrrolizin-7a(5H)-yl) methoxy)-4-(2,2,2-trifluoroethoxy)pyrido[4,3-d]pyrimidine: To a solution of 7-chloro-8-fluoro-2-((tetrahydro-1H-pyrrolizin-7a(5H)-yl)methoxy)-4-(2,2,2-trifluoroethoxy)pyrido[4,3-d]pyrimidine (5.00 g, 1.0 equiv) in CPME (5.0 mL) were added 2-(8-fluoro-1-naphthyl)-4,4,5,5-tetramethyl-1,3,2-dioxaborolane (3.23 g, 1.0 equiv), Cs₂CO₃ (23.8 mL, 3.0 equiv) and CataCXium A Pd G3 (865 mg, 0.1 equiv). The reaction was degassed and purged with nitrogen 3 times. The reaction was stirred at 90 °C for 1 hour. The mixture was diluted with H₂O (100 mL) and extracted with ethyl acetate (3×100 mL). The combined organic layers were dried over anhydrous sodium sulfate, concentrated and purified with column chromatography [SiO₂, Petroleum ether/Ethyl acetate=10/1] to afford the title compound (4.60 g, 67% yield) as white solid; ¹H NMR (400 MHz, DMSO-d₆) δ=9.20 (s, 1H), 8.22-8.18 (m, 1H), 7.95 (d, J=8.0 Hz, 1H), 7.78-7.73 (m, 1H), 7.65 (d, J=6.4 Hz, 1H), 7.60 (dt, J=5.2, 8.0 Hz, 1H), 7.32 (dd, J=7.6, 13.2 Hz, 1H), 5.43-5.31 (m, 2H), 4.18 (s, 2H), 3.01-2.89 (m, 2H), 2.61-2.53 (m, 2H), 1.95-1.87 (m, 2H), 1.84-1.75 (m, 4H), 1.64-1.56 (m, 2H); LCMS (ESI, M+1): m/z=531.4.

Step E. Tert-butyl-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (500 mg, 1.0 equiv) and tert-butyl piperidine-4-carboxylate (627 mg, 3.0 equiv) in DMF (10 mL) were added N-ethyl-N,N-diisopropylamine (1.10 g, 9.0 equiv) and 4 Å molecular sieve (75.0 mg). The reaction was stirred at 50 °C for 16 hours. The mixture was diluted with water (80 mL) and extracted with ethyl acetate (3×30 mL). The combined organic layers were washed with brine (2×20 mL), dried over anhydrous sodium sulfate, concentrated and purified with prep-TLC [(SiO₂, Dichloromethane/Methanol)=12/1] to afford the title compound (400 mg, 69% yield) as light yellow solid; LCMS (ESI, M+1): m/z=616.4.

Step F. 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid: To a solution of tert-butyl 1-(8-fluoro-7-(8-fluoronaphthalen-1-yl)-2-((tetrahydro-1H-pyrrolizin-7a(5H)-yl) methoxy)pyrido[4,3-d]pyrimidin-4-yl)piperidine-4-carboxylate (100 mg, 1.0 equiv) in DCM (5.5 mL) was added TFA (1.8 mL) at 0 °C. The reaction was stirred at 25 °C for 1 hour. The mixture was concentrated to afford title compound (90.0 mg, crude) as light yellow oil; LCMS (ESI, M+1): m/z=560.3.

Step G. 3-(1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carbonyl)oxazolidin-2-one: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (50.0 mg, 1.0 equiv) and oxazolidin-2-one (8.56 mg, 1.1 equiv) in DCM (0.5 mL) were added DMAP (13.6 mg, 1.2 equiv) and EDCI (21.4 mg, 1.2 equiv). The reaction was stirred at 30 °C for 12 hours. The mixture was concentrated and purified by prep-HPLC [column: Phenomenex luna C18 150×25 mm×10 μm; A: water (FA), B: ACN, B %: 22%-52% B over 9 min] and prep-HPLC [column: Phenomenex luna C18 150×25 mm×10 μm; A: water (FA), B: ACN, B %: 20%-50% B over 9 min] to afford the title compound (7.42 mg, 13% yield) as white solid; ¹H NMR (400 MHz, CHLOROFORM-d) δ=9.04 (s, 1H), 7.99 (br d, J=7.6 Hz, 1H), 7.74 (d, J=8.0 Hz, 1H), 7.68-7.58 (m, 2H), 7.45 (dt, J=4.8, 8.0 Hz, 1H), 7.12 (dd, J=7.6, 12.8 Hz, 1H), 4.77-4.63 (m, 2H), 4.57-4.43 (m, 4H), 4.07 (t, J=8.0 Hz, 2H), 4.02-3.91 (m, 1H), 3.60-3.42 (m, 4H), 2.79 (td, J=6.6, 10.8 Hz, 2H), 2.26 (td, J=6.4, 12.0 Hz, 2H), 2.19-2.09 (m, 2H), 2.09-1.92 (m, 6H), 1.89-1.77 (m, 2H); LCMS (ESI, M+1): m/z=629.3.

### Example 3

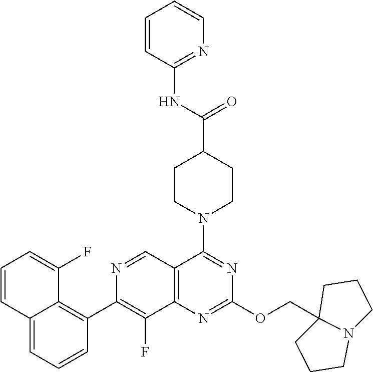 [SMILES: O=C(Nc1ccccn1)C1CCN(c2nc(OCC34CCCN3CCC4)nc3c(F)c(-c4cccc5cccc(F)c45)ncc23)CC1]

### 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-N-(pyridin-2-yl)piperidine-4-carboxamide

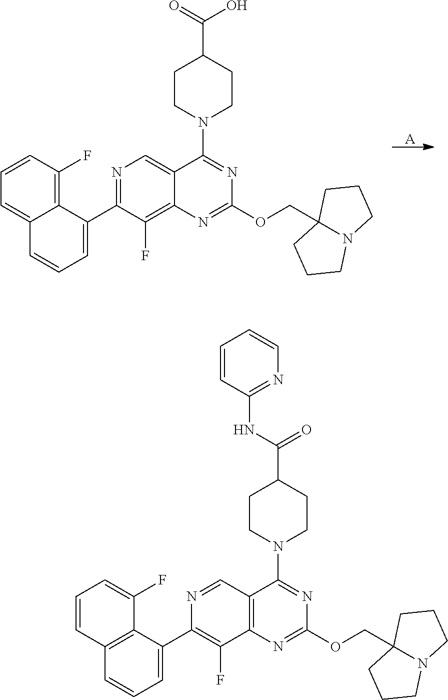

Step A. 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-N-(pyridin-2-yl)piperidine-4-carboxamide: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (200 mg, 1.0 equiv) in DMF (2 mL) were added HATU (272 mg, 2 equiv), DIEA (277 mg, 6.0 equiv) and pyridin-2-amine (37 mg, 1.1 equiv). The reaction was stirred at 25 °C for 1 hour. The mixture was purified by prep-HPLC [Waters Xbridge 150×25 mm×5 μm; A: water (NH₄HCO₃), B: ACN, B %: 34%-64% B over 9 min] to afford the title compound (7.46 mg, 3.2% yield) as white solid; ¹H NMR (400 MHz, CHLOROFORM-d) δ=9.05 (s, 1H), 8.31 (dd, J=0.8, 4.8 Hz, 1H), 8.24 (d, J=8.4 Hz, 1H), 8.14 (s, 1H), 8.01 (td, J=1.8, 7.6 Hz, 1H), 7.78-7.71 (m, 2H), 7.68-7.60 (m, 2H), 7.46 (dt, J=5.2, 7.8 Hz, 1H), 7.17-7.05 (m, 2H), 4.71 (br d, J=13.6 Hz, 2H), 4.27 (s, 2H), 3.53-3.39 (m, 2H), 3.16 (td, J=5.2, 10.0 Hz, 2H), 2.77-2.62 (m, 3H), 2.24-2.10 (m, 6H), 1.94-1.87 (m, 4H), 1.74-1.65 (m, 2H); LCMS (ESI, M+1): m/z=636.3.

### Example 4

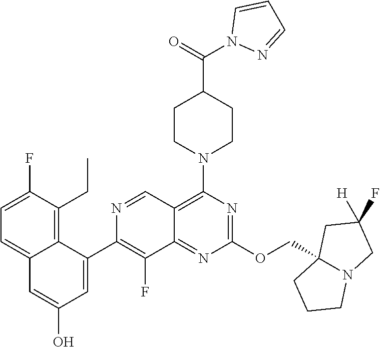 [SMILES: CCc1c(F)ccc2cc(O)cc(-c3ncc4c(N5CCC(C(=O)n6cccn6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(1H-pyrazol-1-yl)methanone

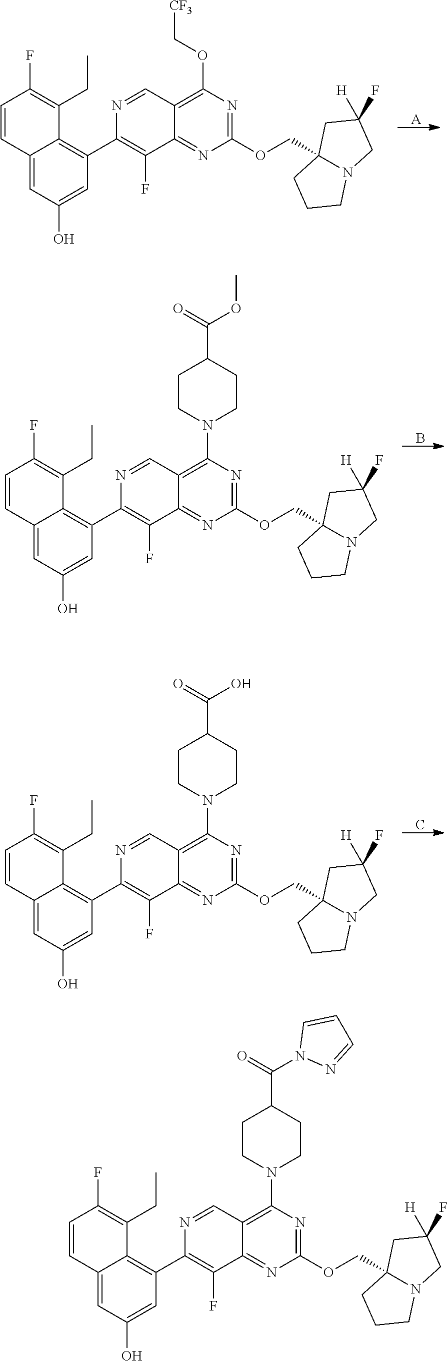

Step A. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (1.0 g, 1.0 equiv) and methyl piperidine-4-carboxylate (290 mg, 1.2 equiv) in DMAC (10 mL) was added K₃PO₄ (1.07 g, 3.0 equiv). The reaction was stirred at 60 °C for 0.5 hours. The mixture was quenched with water (15 mL) and extracted with ethyl acetate (3×10 mL). The combined organic layers were washed with brine (2×30 mL), dried over Na₂SO₄ and concentrated to afford the title compound (1.2 g, crude) as yellow oil.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (1.2 g, 1.0 equiv) in MeOH (4.0 mL) was added LiOH·H₂O (2 M, 3.78 mL, 4.0 equiv). The reaction was stirred at 20 °C for 0.5 hours. The mixture was filtered and purified by reversed phase flash [C18, 0.1% formic acid condition] to afford the title compound (0.8 g, 65.8% yield) as white solid; LCMS (ESI, M+1): m/z=622.3.

Step C. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 equiv) and TEA (81.4 mg, 5.0 equiv) in DMF (2.0 mL) were added EDCI (38.5 mg, 1.25 equiv) and HOBt (2.17 mg, 0.10 equiv). The reaction was stirred at 25 °C for 0.5 hours, and then 1H-pyrazole (43.8 mg, 4.0 equiv) was added. The reaction was added stirred at 25 °C for 48 hours. The mixture was filtered and purified with prep-HPLC [column: Waters Xbridge 150×25 mm×5 μm; A: water (NH₄HCO₃), B: ACN; B %: 48%-78% over 9 min] twice. The desired fractions were extracted with DCM (3×7.0 mL). The combined organic layers were dried over Na₂SO₄ and concentrated to afford the title compound (11.3 mg, 9.89% yield) as a white solid; ¹H NMR (400 MHz, CHLOROFORM-d) δ=8.96 (s, 1H), 8.27 (d, J=2.8 Hz, 1H), 7.77 (d, J=0.8 Hz, 1H), 7.58-7.50 (m, 1H), 7.22-7.14 (m, 2H), 7.06-6.94 (m, 1H), 6.50 (dd, J=1.2, 2.8 Hz, 1H), 5.41-5.20 (m, 1H), 4.74-4.53 (m, 2H), 4.44-4.23 (m, 2H), 4.14-3.98 (m, 1H), 3.61-3.10 (m, 5H), 3.08-2.94 (m, 1H), 2.56-2.39 (m, 1H), 2.38-2.27 (m, 1H), 2.26-2.04 (m, 7H), 2.00-1.89 (m, 3H), 0.82 (br t, J=7.6 Hz, 3H); LCMS (ESI, M+1): m/z=672.3.

### Example 5

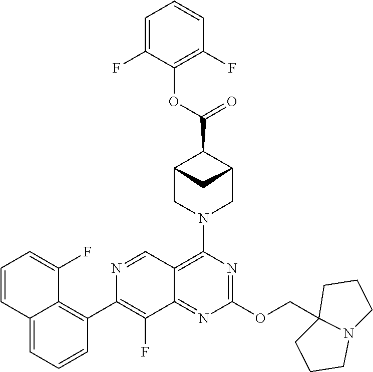 [SMILES: O=C(Oc1c(F)cccc1F)[C@@H]1[C@@H]2C[C@H]1CN(c1nc(OCC34CCCN3CCC4)nc3c(F)c(-c4cccc5cccc(F)c45)ncc13)C2]

### 2,6-difluorophenyl (1R,5S,6r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate

### Example 6

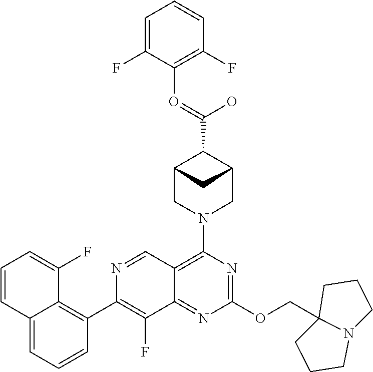

### 2,6-difluorophenyl (1R,5S,6s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate

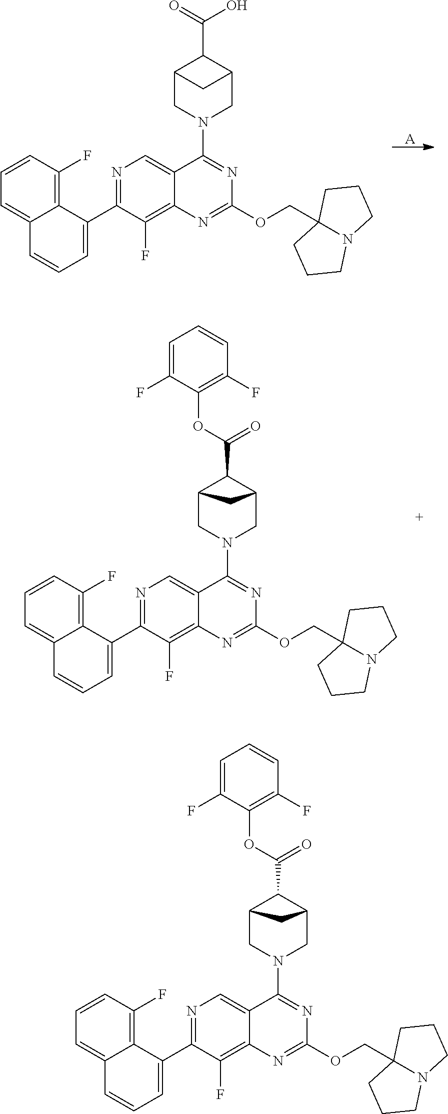

Step A. 2,6-difluorophenyl (1R,5S,6s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate and 2,6-difluorophenyl (1R,5S,6r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate: To a solution of 3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylic acid (150 mg, 1.0 equiv), EDCI (75.5 mg, 1.5 equiv) and DMAP (48.1 mg, 1.5 equiv) in DCM (2 mL) was added 4-fluorophenol (68.3 mg, 2.0 equiv). The reaction was stirred at 25 °C for 4 hours. The mixture was concentrated. The residue was diluted with H₂O (2 mL) and extracted with EtOAc (3×2 mL). The combined organic layers were washed with brine (2 mL), dried over anhydrous sodium sulfate, concentrated and purified with prep-HPLC [column: YMC-Actus Triart C18 150×30 mm×7 μm; A: water (FA), B: ACN; B %: 20%-50% B over 10 min] and lyophilized to afford to afford the two isomers.

2,6-difluorophenyl (1R,5S,6r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate (Example 5, 2.61 mg, 1.3% yield) as yellow-white solid; ¹H NMR (400 MHz, CHLOROFORM-d) δ=9.48-9.43 (m, 1H), 8.02-7.98 (m, 1H), 7.77-7.73 (m, 1H), 7.66-7.60 (m, 2H), 7.48-7.41 (m, 1H), 7.24-7.17 (m, 1H), 7.12 (dd, J=7.6, 12.8 Hz, 1H), 7.02 (s, 2H), 4.66-4.56 (m, 2H), 4.50 (s, 4H), 3.75-3.63 (m, 2H), 3.23 (br d, J=6.0 Hz, 2H), 3.08 (d, J=5.6 Hz, 1H), 2.96-2.89 (m, 2H), 2.36 (br dd, J=6.4, 12.8 Hz, 2H), 2.21-2.13 (m, 2H), 2.13-2.04 (m, 3H), 1.95 (br dd, J=6.8, 13.2 Hz, 2H), 1.66 (br dd, J=6.0, 10.0 Hz, 1H); LCMS (ESI, M+1): m/z=684.4.

2,6-difluorophenyl (1R,5S,6s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate (Example 6, 3.51 mg, 1.8% yield) as yellow solid; ¹H NMR (400 MHz, CHLOROFORM-d) δ=9.50-9.43 (m, 1H), 8.03-7.93 (m, 1H), 7.73 (d, J=8.0 Hz, 1H), 7.66-7.59 (m, 2H), 7.48-7.41 (m, 1H), 7.17-7.07 (m, 2H), 6.95-6.86 (m, 2H), 4.82-4.66 (m, 2H), 4.63-4.53 (m, 2H), 4.37-4.26 (m, 2H), 3.74-3.59 (m, 2H), 3.58-3.48 (m, 1H), 3.23-3.13 (m, 2H), 2.99-2.86 (m, 2H), 2.37-2.33 (m, 2H), 2.16-2.09 (m, 2H), 2.09-2.04 (m, 2H), 2.01 (br d, J=4.0 Hz, 1H), 1.92 (br dd, J=6.8, 13.2 Hz, 2H), 1.67 (d, J=10.0 Hz, 1H); LCMS (ESI, M+1): m/z=684.4.

### Example 7

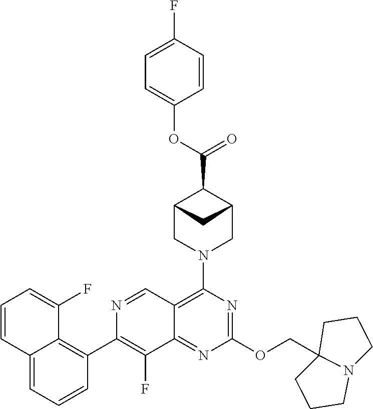 [SMILES: O=C(Oc1ccc(F)cc1)[C@@H]1[C@@H]2C[C@H]1CN(c1nc(OCC34CCCN3CCC4)nc3c(F)c(-c4cccc5cccc(F)c45)ncc13)C2]

### 4-fluorophenyl (1R,5S,6r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate

### Example 8

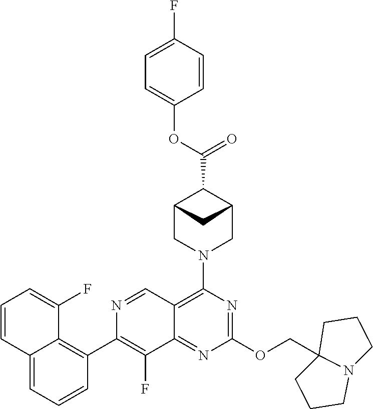 [SMILES: O=C(Oc1ccc(F)cc1)[C@H]1[C@@H]2C[C@H]1CN(c1nc(OCC34CCCN3CCC4)nc3c(F)c(-c4cccc5cccc(F)c45)ncc13)C2]

### 4-fluorophenyl (1R,5S,6s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate

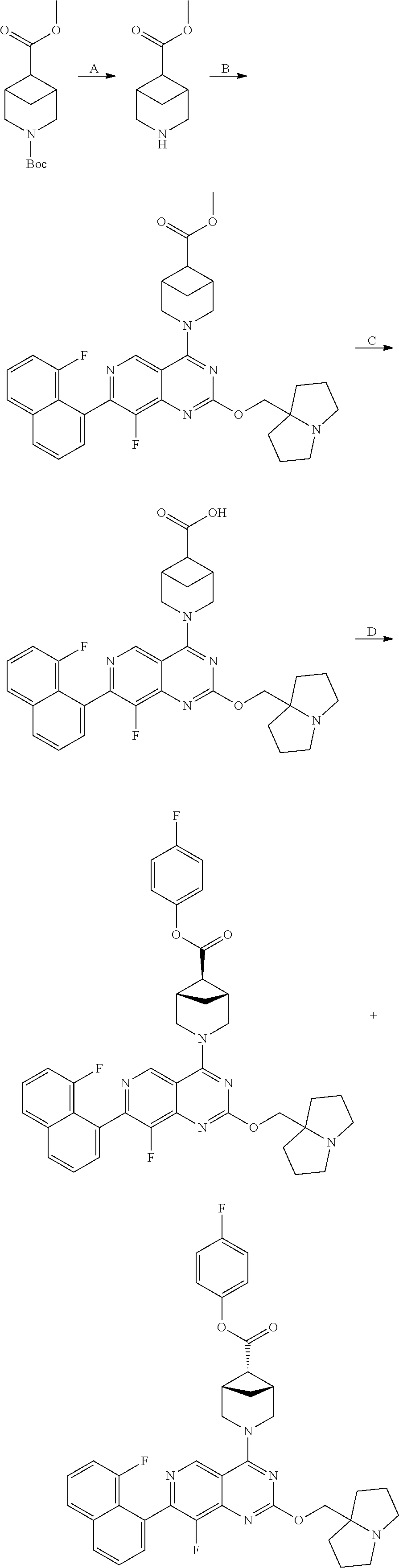

Step A. methyl 3-azabicyclo[3.1.1]heptane-6-carboxylate: To a mixture of 3-(tert-butyl) 6-methyl 3-azabicyclo[3.1.1]heptane-3,6-dicarboxylate (950 mg, 1.0 equiv) in MeCN (10 mL) was added HCl·dioxane (10 mL, 4M). The reaction was stirred at 0 °C for 1 hour. The mixture was concentrated and diluted with MeOH (10 mL). The mixture was adjusted pH to 7 by NaHCO₃solid, filtered and concentrated to afford the title compound (570 mg, 99% yield) as white solid;

Step B. methyl 3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate: To a mixture of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (1.95 g, 1.0 equiv), methyl 3-azabicyclo[3.1.1]heptane-6-carboxylate (570 mg, 1.0 equiv) in DMAc (10 mL) was added K₃PO₄ (2.34 g, 3.0 equiv). The reaction was stirred at 60 °C for 12 hours. The mixture was diluted with H₂O (20 mL) and extracted with EtOAc (3×20 mL). The combined organic layers were washed with brine (20 mL), dried over anhydrous sodium sulfate, concentrated and purified by reversed phase flash chromatography [C18, 0.1% formic acid condition] to afford the title compound (750 mg, 35% yield) as yellow solid; LCMS (ESI, M+1): m/z=586.4;

Step C. 3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylic acid: To a solution of methyl 3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate (100 mg, 1.0 equiv) in MeOH (1 mL) and H₂O (1 mL) was added LiOH·H₂O (179 mg, 25 equiv). The reaction was stirred at 25 °C for 4 hours. The mixture was concentrated and adjust to pH=7 with HCl (4 M). The mixture was filtered to afford the title compound (70.0 mg, 72% yield) as yellow solid; LCMS (ESI, M+1): m/z=572.4;

Step D. 4-fluorophenyl (1R,5S,6s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate and 4-fluorophenyl (1R,5S,6r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate: To a solution of 3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylic acid (35 mg, 1.0 equiv), EDCI (17.6 mg, 1.5 equiv) and DMAP (11.2 mg, 1.5 equiv) in DCM (1 mL) was added 4-fluorophenol (13.7 mg, 2.0 equiv). The reaction was stirred at 25 °C for 4 hours. The mixture was concentrated. The residue was diluted with H₂O (2 mL) and extracted with EtOAc (3×2 mL). The combined organic layers were washed with brine (2 mL), dried over anhydrous sodium sulfate, concentrated and purified with prep-HPLC [column: Welch Xtimate C18 150×25 mm×5 μm; A: water (FA), B: ACN; B %: 30%-50% B over 10 min] to afford two isomers.

4-fluorophenyl (1R,5S,6r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate (Example 7, 2.05 mg, 4.7% yield) as yellow-white solid; ¹H NMR (400 MHz, CHLOROFORM-d) δ=9.50-9.43 (m, 1H), 8.04-7.96 (m, 1H), 7.78-7.72 (m, 1H), 7.67-7.58 (m, 2H), 7.50-7.42 (m, 1H), 7.16-7.11 (m, 3H), 7.10 (d, J=2.4 Hz, 2H), 4.90-4.81 (m, 2H), 4.57-4.47 (m, 4H), 3.96-3.84 (m, 2H), 3.24-3.15 (m, 2H), 3.03-2.93 (m, 3H), 2.86-2.78 (m, 1H), 2.51-2.41 (m, 2H), 2.33-2.21 (m, 2H), 2.20-2.10 (m, 2H), 2.09-2.00 (m, 2H), 1.29-1.24 (m, 1H); LCMS (ESI, M+1): m/z=666.4

4-fluorophenyl (1R,5S,6s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.1.1]heptane-6-carboxylate (Example 8, 2.38 mg, 5.5% yield) as off-white solid; ¹H NMR (400 MHz, CHLOROFORM-d) δ=9.49-9.43 (m, 1H), 8.03-7.96 (m, 1H), 7.77-7.71 (m, 1H), 7.67-7.57 (m, 2H), 7.49-7.41 (m, 1H), 7.11 (dd, J=7.6, 12.8 Hz, 1H), 7.00 (s, 2H), 6.93-6.84 (m, 2H), 4.91-4.62 (m, 4H), 4.44-4.26 (m, 2H), 3.98-3.72 (m, 2H), 3.47 (br d, J=2.8 Hz, 1H), 3.23-3.07 (m, 2H), 3.02-2.84 (m, 2H), 2.44-2.29 (m, 3H), 2.26-2.15 (m, 2H), 2.14-2.05 (m, 2H), 2.04-1.91 (m, 2H), 1.66 (br s, 1H); LCMS (ESI, M+1): m/z=666.4.

### Example 9

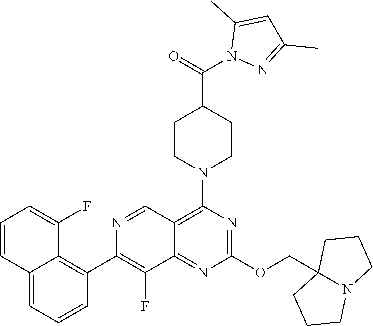 [SMILES: Cc1cc(C)n(C(=O)C2CCN(c3nc(OCC45CCCN4CCC5)nc4c(F)c(-c5cccc6cccc(F)c56)ncc34)CC2)n1]

### (3,5-dimethyl-1H-pyrazol-1-yl)(1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)methanone

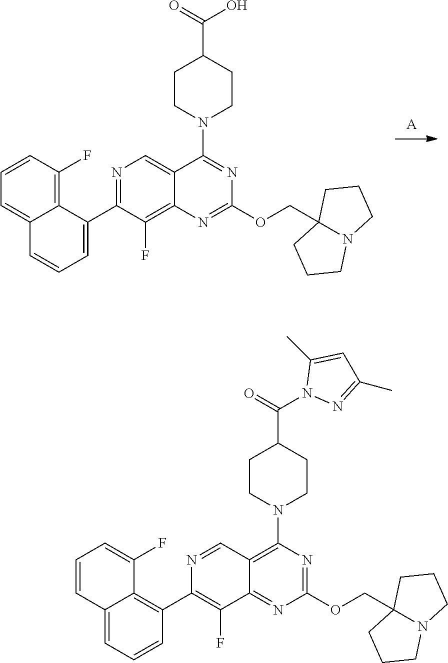

Step A. (3,5-dimethyl-1H-pyrazol-1-yl)(1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)methanone: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (50 mg, 1.0 equiv) in DCM (0.5 mL) were added 3,5-dimethyl-1H-pyrazole (25.8 mg, 3.0 equiv), EDCI (25.7 mg, 1.5 equiv) and DMAP (16.4 mg, 1.5 equiv). The reaction was stirred at 25 °C for 1 hour. The mixture was concentrated. The residue was diluted with water (1 mL), neutralized with solid NaHCO₃ and extracted with ethyl acetate (2×2 mL). The combined organic layers were dried over anhydrous sodium sulfate, concentrated and purified by prep-HPLC [Phenomenex luna C18 150×25 mm×10 μm; A: water (FA); B: ACN, B %: 30%-60% over 7 min] to afford the title compound (16.4 mg, 28% yield, HCOOH salt) as white solid; ¹H NMR (400 MHz, DMSO-d6) δ=9.11 (s, 1H), 8.19 (br d, J=8.4 Hz, 1H), 7.94 (d, J=8.0 Hz, 1H), 7.78-7.72 (m, 1H), 7.65 (d, J=6.8 Hz, 1H), 7.59 (dt, J=5.2, 8.0 Hz, 1H), 7.32 (dd, J=7.6, 13.2 Hz, 1H), 6.23 (s, 1H), 4.58 (br d, J=13.2 Hz, 2H), 4.08 (s, 2H), 4.07-3.99 (m, 1H), 3.57 (br t, J=12.8 Hz, 2H), 2.98-2.90 (m, 2H), 2.59-2.53 (m, 2H), 2.49 (s, 3H), 2.23 (s, 3H), 2.18-2.09 (m, 2H), 1.99-1.87 (m, 4H), 1.85-1.71 (m, 4H), 1.63-1.54 (m, 2H); LCMS (ESI, M+1): m/z=638.4.

### Example 10

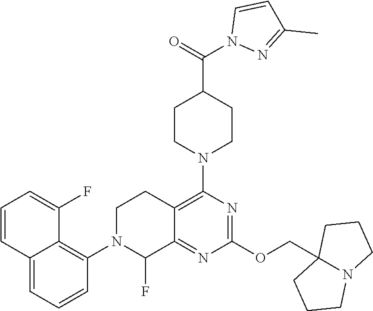 [SMILES: Cc1ccn(C(=O)C2CCN(c3nc(OCC45CCCN4CCC5)nc4c3CCN(c3cccc5cccc(F)c35)C4F)CC2)n1]

### (1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)(3-methyl-1H-pyrazol-1-yl)methanone

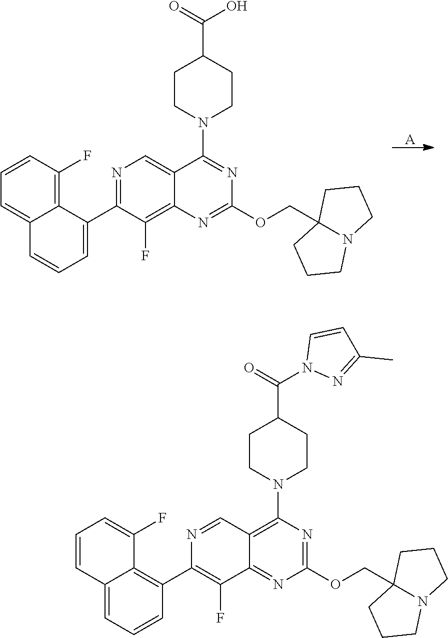

Step A. (1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)(3-methyl-1H-pyrazol-1-yl)methanone:

To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (50 mg, 1.0 equiv) in DCM (0.5 mL) were added 5-methyl-1H-pyrazole (22.0 mg, 3.0 equiv), EDCI (25.7 mg, 1.5 equiv) and DMAP (16.4 mg, 1.5 equiv). The reaction was stirred at 25 °C for 1 hour. The mixture was concentrated. The residue was diluted with water (1 mL), neutralized with solid NaHCO₃ and extracted with ethyl acetate (2×2 mL). The combined organic layers were dried over anhydrous sodium sulfate, concentrated and purified by prep-HPLC [Phenomenex luna C18 150×25 mm×10 μm; A: water (FA); B: ACN, B %: 28%-58% B over 7 min] to afford the title compound (15.1 mg, 27% yield) as white solid; ¹H NMR (400 MHz, DMSO-d6) δ=9.12 (s, 1H), 8.32 (d, J=2.8 Hz, 1H), 8.19 (br d, J=8.4 Hz, 1H), 7.94 (d, J=8.0 Hz, 1H), 7.79-7.72 (m, 1H), 7.65 (d, J=6.4 Hz, 1H), 7.59 (dt, J=5.2, 7.6 Hz, 1H), 7.32 (dd, J=7.6, 13.2 Hz, 1H), 6.50 (d, J=2.8 Hz, 1H), 4.59 (br d, J=13.2 Hz, 2H), 4.08 (s, 2H), 4.05-3.95 (m, 1H), 3.59 (br t, J=12.8 Hz, 2H), 2.99-2.88 (m, 2H), 2.58-2.53 (m, 2H), 2.31 (s, 3H), 2.19-2.11 (m, 2H), 2.01-1.86 (m, 4H), 1.86-1.71 (m, 4H), 1.63-1.53 (m, 2H); LCMS (ESI, M+1): m/z=624.4.

### Example 11

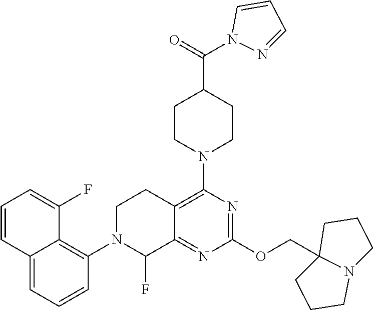 [SMILES: O=C(C1CCN(c2nc(OCC34CCCN3CCC4)nc3c2CCN(c2cccc4cccc(F)c24)C3F)CC1)n1cccn1]

### (1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)(1H-pyrazol-1-yl)methanone

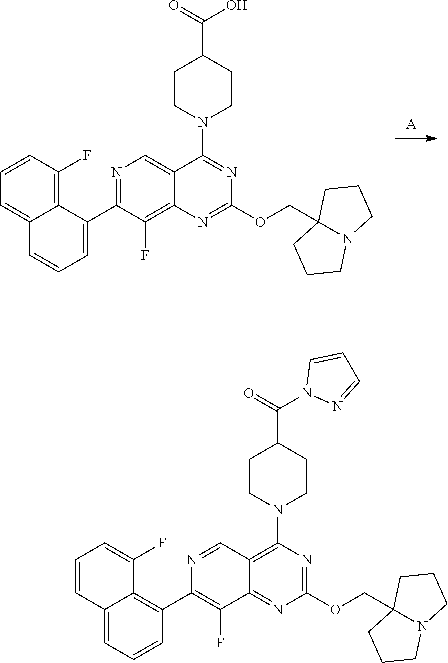

Step A. (1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)(1H-pyrazol-1-yl)methanone: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (150 mg, 1.0 equiv) in DCM (1.5 mL) were added 1H-pyrazole (54.7 mg, 3.0 equiv), EDCI (77.1 mg, 1.5 equiv) and DMAP (49.1 mg, 1.5 equiv). The reaction was stirred at 25 °C for 1 hour. The mixture was diluted with water (5 mL) and extracted with DCM (10 mL). The combined organic layer was dried over sodium sulfate, concentrated under and purified by prep-HPLC. and purified with prep-HPLC [Phenomenex luna C18 150×25 mm×10 μm; A: water (FA); B: ACN, B %: 18%-48% B over 10 min] to afford the title compound (14.4 mg, 8% yield) as white solid; ¹H NMR (400 MHz, CDCl₃) δ=9.10 (s, 1H), 8.29 (d, J=4.8 Hz, 1H), 7.99 (d, J=7.6 Hz, 1H), 7.77-7.73 (m, 2H), 7.63-7.59 (m, 3H), 7.50-7.40 (m, 1H), 7.14-7.09 (m, 1H), 6.52 (s, 1H), 4.76-4.72 (m, 2H), 4.29 (s, 2H), 4.15-4.00 (m, 1H), 3.51 (br t, J=12.0 Hz, 2H), 3.20-3.18 (m, 2H), 2.69-2.68 (m, 2H), 2.23-2.22 (m, 2H), 2.16-2.12 (m, 4H), 1.93-1.88 (m, 4H), 1.71-1.60 (m, 2H); LCMS (ESI, M+1): m/z=610.4.

### Example 12

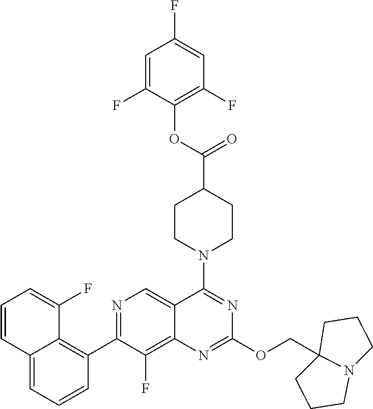 [SMILES: O=C(Oc1c(F)cc(F)cc1F)C1CCN(c2nc(OCC34CCCN3CCC4)nc3c(F)c(-c4cccc5cccc(F)c45)ncc23)CC1]

### 2,4,6-trifluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

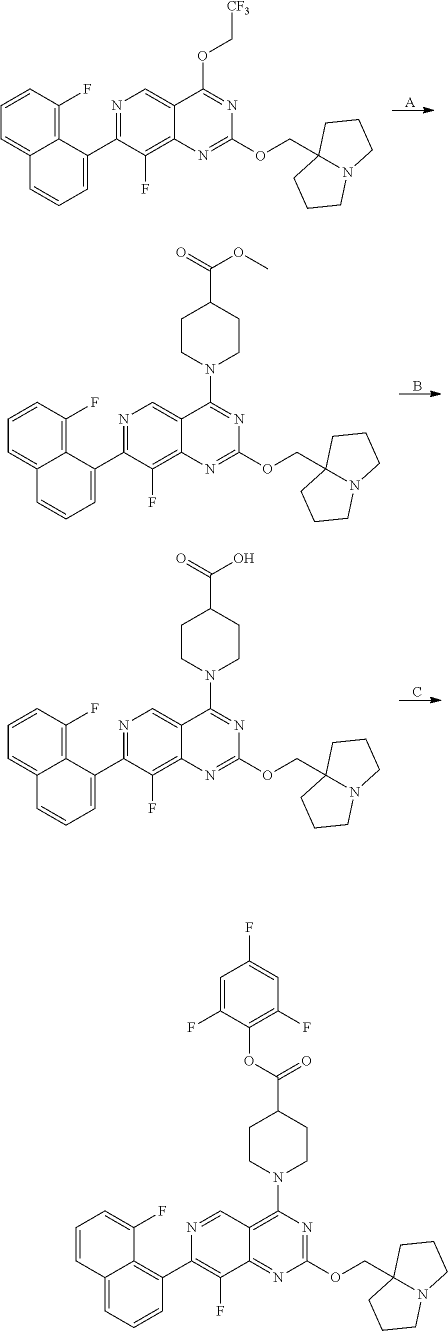

Step A. methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (2.00 g, 1.0 equiv) in DMAC (20 mL) were added K₃PO₄ (2.40 g, 3.0 equiv) and methyl piperidine-4-carboxylate (1.62 g, 3.0 equiv). The reaction was stirred at 60 °C for 1 hour. The mixture was filtered, concentrated and purified by reversed phase flash [C18, 0.1% formic acid condition] to afford the title compound (2.07 g, 92% yield) as white solid; LCMS (ESI, M+1): m/z=574.3.

Step B. 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid: To a solution of methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate (2.07 g, 1.0 equiv) in MeOH (21 mL) was added LiOH·H₂O (757 mg, 5.0 equiv) and H₂O (7 mL). The reaction was stirred at 25 °C for 1 hour. The mixture was concentrated and purified by reversed phase flash [C18, 0.1% formic acid condition] to afford the title compound (1.49 g, 68% yield) as white solid; LCMS (ESI, M+1): m/z=560.3.

Step C. 2,4,6-trifluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (80.0 mg, 1.0 equiv) and 2,4,6-trifluorophenol (63.5 mg, 3.0 equiv) in DCM (2 mL) were added DMAP (26.2 mg, 1.5 equiv) and EDCI (41.1 mg, 1.5 equiv). The reaction was stirred at 30 °C for 1 hour. The mixture was concentrated and purified by prep-HPLC [column: Welch Ultimate XB-SiOH 250×50 mm×10 μm; A: Hexane, B: EtOH, B %: 5%-45% B over 15 min] and prep-HPLC [column: Welch Xtimate C18 150×25 mm×5 μm; A: water (FA), B: ACN, B %: 25%-55% B over 10 min] to afford the title compound (3.52 mg, 3.3% yield) as white solid; ¹H NMR (400 MHz, CHLOROFORM-d) δ=9.06 (s, 1H), 8.01 (br d, J=7.6 Hz, 1H), 7.76 (d, J=8.0 Hz, 1H), 7.69-7.57 (m, 2H), 7.47 (dt, J=4.8, 8.0 Hz, 1H), 7.14 (dd, J=7.6, 12.8 Hz, 1H), 6.90-6.74 (m, 2H), 4.55 (br dd, J=4.0, 13.2 Hz, 2H), 4.36 (s, 2H), 3.77-3.57 (m, 2H), 3.34-3.22 (m, 2H), 3.20-3.05 (m, 1H), 2.83-2.64 (m, 2H), 2.44-2.28 (m, 2H), 2.27-2.09 (m, 4H), 2.07-1.87 (m, 4H), 1.83-1.66 (m, 2H); LCMS (ESI, M+1): m/z=690.3.

### Example 13

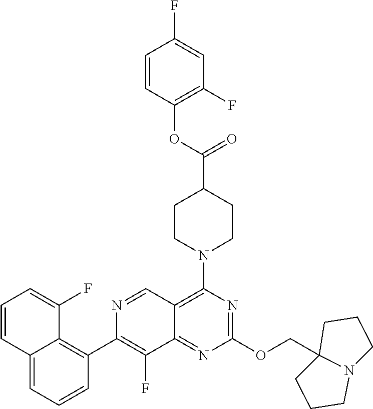 [SMILES: O=C(Oc1ccc(F)cc1F)C1CCN(c2nc(OCC34CCCN3CCC4)nc3c(F)c(-c4cccc5cccc(F)c45)ncc23)CC1]

### 2,4-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

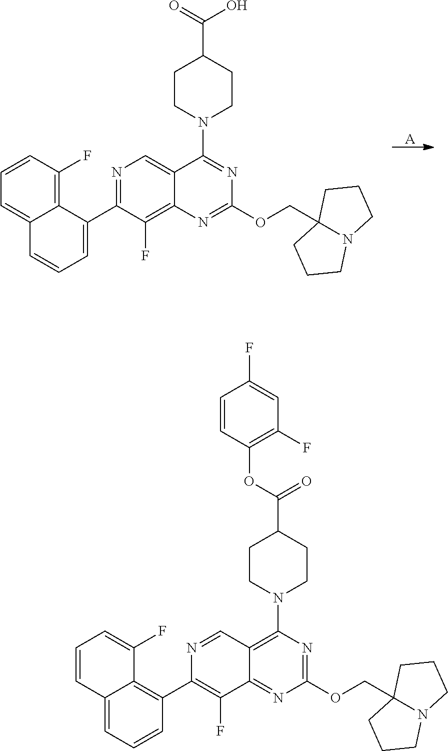

Step A. 2,4-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (50.0 mg, 1.0 equiv) and 2,4-difluorophenol (34.9 mg, 3.0 equiv) in DCM (1 mL) were added DMAP (16.4 mg, 1.5 equiv) and EDCI (25.7 mg, 1.5 equiv). The reaction was stirred at 30 °C for 1 hour. The mixture was quenched with water (5.0 mL) and extracted with ethyl acetate (3×5 mL). The combined organic layers were concentrated and purified by prep-TLC (Dichloromethane:Methanol=8/1) and prep-HPLC [column: Welch Xtimate C18 150×25 mm×5 μm; A: water (FA), B: ACN, B %: 23%-53% B over 10 min] to afford the title compound (2.19 mg, 3.6% yield) as white solid; ¹H NMR (400 MHz, CHLOROFORM-d) δ=9.05 (s, 1H), 8.05-7.95 (m, 1H), 7.75 (d, J=8.0 Hz, 1H), 7.68-7.57 (m, 2H), 7.50-7.41 (m, 1H), 7.18-7.07 (m, 2H), 7.01-6.84 (m, 2H), 4.55 (br dd, J=4.4, 9.2 Hz, 2H), 4.39-4.23 (m, 2H), 3.68-3.54 (m, 2H), 3.29-3.01 (m, 3H), 2.77-2.61 (m, 2H), 2.38-2.25 (m, 2H), 2.24-2.07 (m, 4H), 1.96-1.87 (m, 4H), 1.74-1.66 (m, 2H); LCMS (ESI, M+1): m/z=672.3.

### Example 14

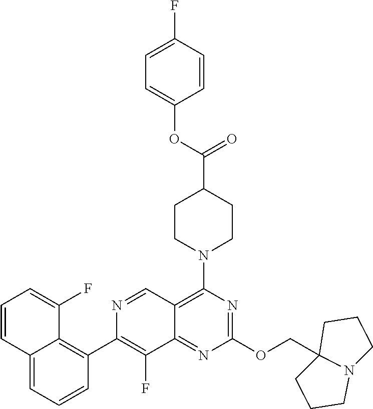 [SMILES: O=C(Oc1ccc(F)cc1)C1CCN(c2nc(OCC34CCCN3CCC4)nc3c(F)c(-c4cccc5cccc(F)c45)ncc23)CC1]

### 4-fluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

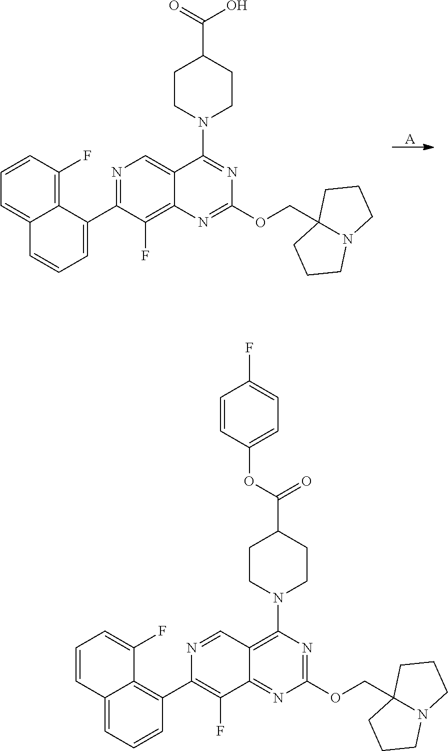

Step A. 4-fluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (200 mg, 1.0 equiv) and 4-fluorophenol (80.1 mg, 2.0 equiv) in DCM (2 mL) were added DMAP (65.5 mg, 1.5 equiv) and EDCI (103 mg, 1.5 equiv). The reaction was stirred at 30 °C for 1 hour. The mixture was quenched with water (5.0 mL) and extracted with ethyl acetate (3×5 mL). The combined organic layers were concentrated and purified with prep-HPLC [column: Phenomenex luna C18 150×25 mm×10 μm; A: water (FA), B: ACN, B %: 30%-60% B over 9 min], prep-TLC (Dichloromethane:Methanol=8/1) and prep-HPLC [column: Welch Xtimate C18 150×25 mm×5 μm; A: water (FA), B: ACN, B %: 20%-50% B over 10 min] to afford the title compound (2.88 mg, 1.2% yield) as white solid; ¹H NMR (400 MHz, CHLOROFORM-d) δ=9.05 (s, 1H), 8.00 (br d, J=7.6 Hz, 1H), 7.75 (d, J=8.4 Hz, 1H), 7.68-7.59 (m, 2H), 7.45 (dt, J=4.8, 8.0 Hz, 1H), 7.13 (br d, J=12.4 Hz, 1H), 7.09 (d, J=2.4 Hz, 2H), 7.08 (s, 2H), 4.66-4.54 (m, 2H), 4.34 (s, 2H), 3.63-3.49 (m, 2H), 3.32-3.19 (m, 2H), 3.07-2.95 (m, 1H), 2.70 (td, J=7.2, 10.4 Hz, 2H), 2.30 (br dd, J=2.8, 13.6 Hz, 2H), 2.22-2.07 (m, 5H), 1.95-1.91 (m, 4H), 1.78-1.69 (m, 2H); LCMS (ESI, M+1): m/z=654.2.

### Example 15

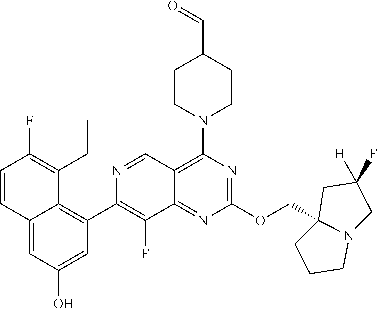 [SMILES: CCc1c(F)ccc2cc(O)cc(-c3ncc4c(N5CCC(C=O)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carbaldehyde

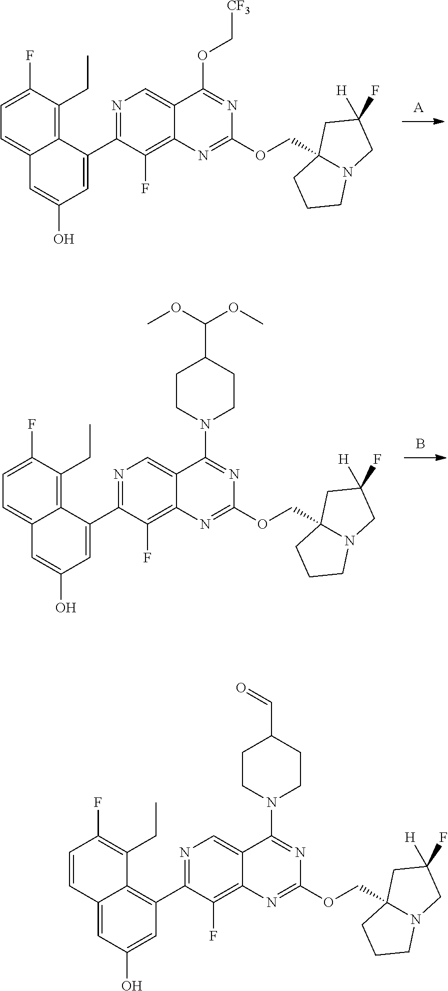

Step A. 4-(4-(4-(dimethoxymethyl)piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (200 mg, 1.0 equiv), 4-(dimethoxymethyl)piperidine (161 mg, 3.0 equiv) and 4 Å molecular sieve (20 mg) in DMF (1 mL) was added DIEA (131 mg, 3.0 equiv). The reaction was stirred at 60 °C for 2 hours. The mixture was filtered and the filtrate was purified with reversed phase flash chromatography [C18, 0.1% formic acid condition] to afford the title compound (150 mg, 65% yield) as yellow solid; LCMS (ESI, M+1): m/z=652.4.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carbaldehyde: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (120 mg, 1 equiv) in DCM (100 μL) was added TFA (420 mg, 20 equiv). The reaction was stirred at 25 °C for 2 hours. The mixture was concentrated. The residue was diluted with saturated NaHCO₃ aqueous solution (10 mL) and extracted with ethyl acetate (3×10 mL). The combined organic layers were dried over anhydrous sodium sulfate, concentrated and purified with prep-HPLC [YMC Triart 30×150 mm×7 μm; A: water (FA); B: ACN, B %: 25%-55% over 8 min] and prep-HPLC [C18 150×30 mm; A: water (FA); B: ACN, B %: 20%-50% over 7 min] to afford the title compound (85.0 mg, 75% yield, HCOOH salt) as white solid; ¹H NMR (400 MHz, METHANOL-d₄) δ 9.09-9.02 (m, 1H), 7.68 (dd, J=5.6, 8.8 Hz, 1H), 7.33-7.29 (m, 1H), 7.28-7.21 (m, 1H), 7.08-7.03 (m, 1H), 5.51-5.30 (m, 1H), 4.79-4.68 (m, 2H), 4.51-4.38 (m, 2H), 4.38-4.32 (m, 1H), 3.61-3.37 (m, 5H), 3.24-3.14 (m, 1H), 2.56-2.33 (m, 3H), 2.29-2.08 (m, 4H), 2.07-1.88 (m, 4H), 1.69-1.53 (m, 2H), 0.85-0.76 (m, 3H); LCMS (ESI, M+1): m/z=606.3.

### Example 16

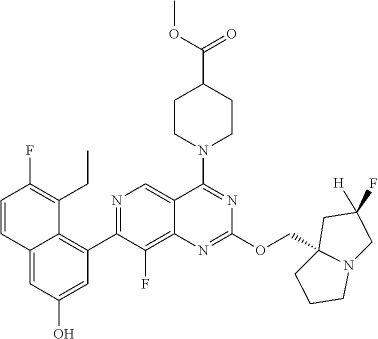 [SMILES: CCc1c(F)ccc2cc(O)cc(-c3ncc4c(N5CCC(C(=O)OC)CC5)nc(OC[C@@]56CCCN5C[C@H](F)C6)nc4c3F)c12]

### methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate

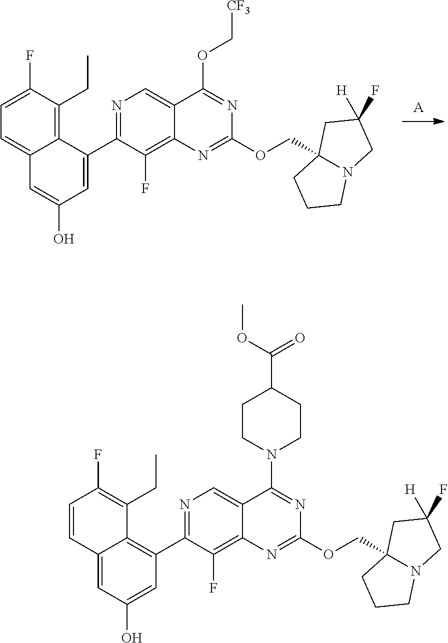

Step A. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (100 mg, 1.0 equiv), methyl piperidine-4-carboxylate (48.3 mg, 2.0 equiv) and 4 Å molecular sieve (20 mg) in DMSO (1 mL) was added DIEA (65.4 mg, 3.0 equiv). The reaction was stirred at 40 °C for 12 hours. The mixture was filtered and the filtrate was purified by reversed phase flash chromatography [C18, 0.1% formic acid condition] and prep-HPLC [Waters Xbridge 150×25 mm×5 μm; A: water (NH₄HCO₃), B: ACN, B: 48%-78% over 9 min] to afford the title compound (22.4 mg, 21% yield) as white solid; ¹H NMR (400 MHz, METHANOL-d₄) δ 9.04 (s, 1H), 7.68 (dd, J=6.0, 9.2 Hz, 1H), 7.30 (d, J=2.4 Hz, 1H), 7.25 (t, J=9.2 Hz, 1H), 7.05 (d, J=2.4 Hz, 1H), 5.40-5.21 (m, 1H), 4.64-4.53 (m, 2H), 4.35-4.22 (m, 2H), 3.72 (s, 3H), 3.67-3.54 (m, 2H), 3.29-3.16 (m, 3H), 3.06-2.97 (m, 1H), 2.91-2.81 (m, 1H), 2.54-2.40 (m, 1H), 2.39-2.09 (m, 6H), 2.04-1.84 (m, 5H), 0.85-0.74 (m, 3H); LCMS (ESI, M+1): m/z=636.4.

### Example 17

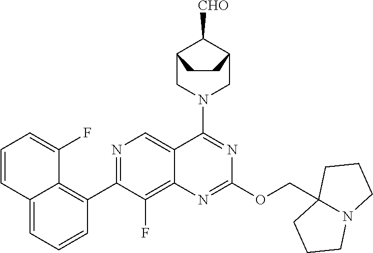 [SMILES: O=C[C@@H]1[C@@H]2CC[C@H]1CN(c1nc(OCC34CCCN3CCC4)nc3c(F)c(-c4cccc5cccc(F)c45)ncc13)C2]

### (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carbaldehyde

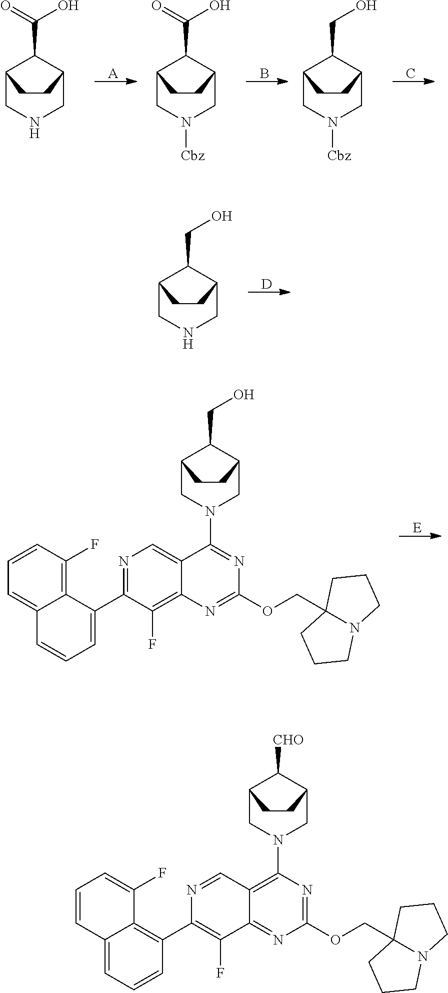

Step A. (1R,5S,8r)-3-((benzyloxy)carbonyl)-3-azabicyclo[3.2.1]octane-8-carboxylic acid; To a mixture of (1R,5S,8r)-3-azabicyclo[3.2.1]octane-8-carboxylic acid (780 mg, 1.0 equiv) and NaOH (221 mg, 1.1 equiv) in H₂O (10.0 mL) was added CbzCl (1.07 g, 1.2 equiv) at 0 °C. The reaction was stirred at 0-10 °C for 16 hours. The mixture was diluted with water (20 mL) and extracted with ethyl acetate (2×30 mL). The combined organic layers were dried over anhydrous sodium sulfate, and concentrated and purified with reversed phase flash chromatography [C18, 0.1% formic acid condition] to afford the title compound (503 mg, 20% yield) as yellow oil; ¹H NMR (400 MHz, CHLOROFORM-d) δ=7.37-7.30 (m, 5H), 5.14 (s, 2H), 4.72 (s, 1H), 4.07-3.88 (m, 2H), 3.05-2.91 (m, 2H), 2.69-2.56 (m, 2H), 1.88-1.75 (m, 2H), 1.65-1.45 (m, 2H); LCMS (ESI, M+1): m/z=290.0

Step B. benzyl (1R,5S,8r)-8-(hydroxymethyl)-3-azabicyclo[3.2.1]octane-3-carboxylate: To a solution of (1R,5S,8r)-3-((benzyloxy)carbonyl)-3-azabicyclo[3.2.1]octane-8-carboxylic acid (300 mg, 1.0 equiv) in THF (3.00 mL) was added BH₃·Me₂S (10 M, 3.0 equiv) at 0 °C. The reaction was stirred at 0 °C for 2 hours. The mixture was quenched with MeOH (5.00 mL) and concentrated under vacuum. The mixture was diluted with water (20 mL) and extracted with ethyl acetate (2×10 mL). The combined organic layers were dried over anhydrous sodium sulfate, and concentrated and purified with prep-HPLC [column: YMC-Gel SiL-HG 250 mm×70 mm×10 μm; A: Hexane, B: EtOH, B %: 5%-45% over 15 min] to afford the title compound (190 mg, 60% yield) as colorless oil; LCMS (ESI, M+1): m/z=276.1

Step C. ((1R,5S,8r)-3-azabicyclo[3.2.1]octan-8-yl)methanol: To a solution of benzyl (1R,5S,8r)-8-(hydroxymethyl)-3-azabicyclo[3.2.1]octane-3-carboxylate (170 mg, 1.0 equiv) in MeOH (5.00 mL) was added Pd/C (20 mg, 10% purity) under N₂ atmosphere. The suspension was degassed and purged with H₂ for 3 times. The reaction was stirred under H₂ (15 Psi) at 30 °C for 2 hours. The mixture was filtered and concentrated to afford the title compound (60 mg, crude) as white solid; LCMS (ESI, M+1): m/z=142.1

Step D. ((1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octan-8-yl)methanol: To a mixture of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (130 mg, 1.0 equiv) and ((1R,5S,8r)-3-azabicyclo[3.2.1]octan-8-yl)methanol (51.9 mg, 1.5 equiv) in DMF (3.00 mL) were added K₃PO₄ (156 mg, 3.0 equiv) and 4 Å molecular sieve (20 mg). The reaction was stirred at 40 °C for 2 hours. The mixture was diluted with water (20 mL) and extracted with ethyl acetate (2×10 mL). The combined organic layers were dried over anhydrous sodium sulfate, concentrated and purified with reversed phase flash chromatography [C18, 0.1% formic acid condition] to afford the title compound (45 mg, 30% yield) as white solid; LCMS (ESI, M+1): m/z=572.4

Step E. (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carbaldehyde; To a solution of ((1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octan-8-yl)methanol (20.0 mg, 1.0 equiv) in DCM (1.00 mL) was added DMP (22.3 mg, 1.5 equiv) at 0 °C. The reaction was stirred at 0 °C for 2 hours. The mixture was quenched with saturated NaHCO₃ solution (10.0 mL) and extracted with ethyl acetate (2×5 mL). The combined organic layers were dried over anhydrous sodium sulfate, concentrated and purified with reversed phase flash chromatography [C18, 0.1% formic acid condition] to afford the title compound (7.41 mg, 33% yield, CF₃COOH salt) as white solid; ¹H NMR (400 MHz, METHANOL-d4) δ=9.07 (s, 1H), 8.11 (br d, J=8.4 Hz, 1H), 7.85 (d, J=8.0 Hz, 1H), 7.74-7.67 (m, 1H), 7.60 (d, J=7.2 Hz, 1H), 7.53 (dt, J=5.2, 8.0 Hz, 1H), 7.25-7.14 (m, 1H), 4.83-4.70 (m, 2H), 4.40 (s, 2H), 4.25 (d, J=8.4 Hz, 1H), 3.68-3.55 (m, 2H), 2.97-2.84 (m, 2H), 2.50 (br s, 2H), 2.23-2.11 (m, 2H), 2.11-1.93 (m, 5H), 1.92-1.82 (m, 2H), 1.82-1.72 (m, 2H), 1.65-1.52 (m, 2H); LCMS (ESI, M+1): m/z=570.3.

### Example 18

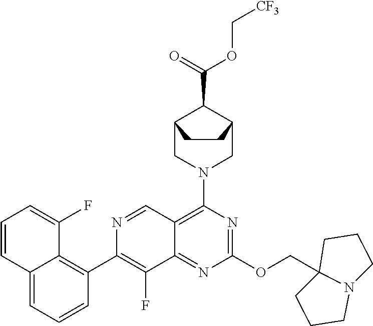 [SMILES: O=C(OCC(F)(F)F)[C@@H]1[C@@H]2CC[C@H]1CN(c1nc(OCC34CCCN3CCC4)nc3c(F)c(-c4cccc5cccc(F)c45)ncc13)C2]

### 2,2,2-trifluoroethyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

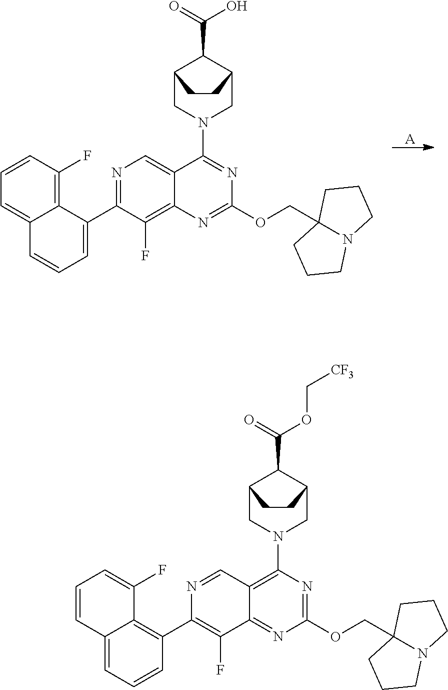

Step A. 2,2,2-trifluoroethyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (50.0 mg, 1.0 equiv) and 2,2,2-trifluoroethanol (25.6 mg, 3.0 equiv) in DCM (1 mL) were added DMAP (15.6 mg, 1.5 eq) and EDCI (24.5 mg, 1.5 equiv). The reaction was stirred at 30 °C for 2 hours. The reaction was concentrated and purified with prep-HPLC [Phenomenex Luna C18 150×25 mm×10 μm; A: water (TFA); B: ACN; B %: 15%-45% over 9 min]. The desired fraction was collect. The mixture was adjusted to pH=7 with saturated NaHCO₃ (10 mL) and extracted with ethyl acetate (2×10 mL). The combined organic layers were dried over Na₂SO₄, concentrated and purified with prep-HPLC [Phenomenex luna C18 150×25 mm×10 μm; A: water (FA); B: ACN; B %: 28%-58% over 9 min] to afford the title compound (19.1 mg, 31% yield) as white solid; ¹H NMR (400 MHz, DMSO-d6) δ=9.22-9.08 (m, 1H), 8.19 (dd, J=8.0 Hz, 1H), 7.94 (t, J=7.6 Hz, 1H), 7.81-7.71 (m, 1H), 7.68-7.55 (m, 2H), 7.36-7.27 (dd, J=7.2, 13.2 Hz, 2H), 4.82 (q, J=9.2 Hz, 2H), 4.63 (br d, J=10.0 Hz, 2H), 4.13 (s, 2H), 3.68 (br d, J=12.8 Hz, 2H), 3.15 (s, 1H), 3.07-2.92 (m, 2H), 2.74 (br s, 2H), 2.67-2.60 (m, 2H), 2.00-1.89 (m, 2H), 1.88-1.74 (m, 4H), 1.74-1.50 (m, 6H); LCMS (ESI, M+1): m/z=668.4.

### Example 19

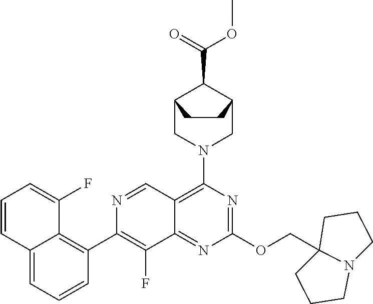 [SMILES: COC(=O)[C@@H]1[C@@H]2CC[C@H]1CN(c1nc(OCC34CCCN3CCC4)nc3c(F)c(-c4cccc5cccc(F)c45)ncc13)C2]

### methyl (1R,5S,8s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

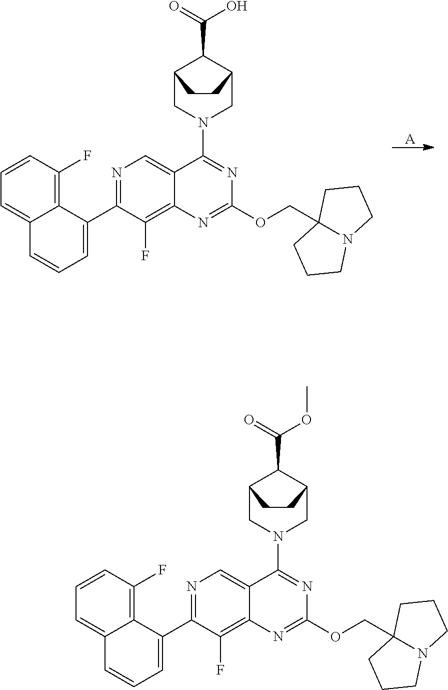

Step A. methyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (90 mg, 1.0 equiv), 4-hydroxybenzonitrile (36.6 mg, 2.0 equiv) and EDCI (44.2 mg, 1.5 equiv) in DCM (2.00 mL) was added DMAP (28.2 mg, 1.5 equiv). The reaction was stirred at 40 °C for 1 hour. MeOH (1 mL, 160 equiv) was added into the mixture. The reaction was stirred at 60 °C for 2 hours. The mixture wash diluted with water (30 mL) and extracted with DCM (2×15 mL). The combined organic layers were dried over anhydrous sodium sulfate, and concentrated and purified with prep-HPLC [column: Welch Ultimate XB-SiOH 250×50×10 μm; A: Hexane-EtOH; B: 10%-50% over 15 min] to afford the title compound (23.7 mg, 25% yield) as white solid; ¹H NMR (400 MHz, METHANOL-d4) δ=9.14-9.07 (m, 1H), 8.18 (br d, J=8.4 Hz, 1H), 7.93 (d, J=8.0 Hz, 1H), 7.77-7.70 (m, 1H), 7.65-7.62 (m, 1H), 7.58 (dt, J=5.2, 8.0 Hz, 1H), 7.31 (dd, J=7.6, 13.2 Hz, 1H), 4.59 (br d, J=12.4 Hz, 2H), 4.07 (s, 2H), 3.64 (s, 4H), 3.62 (s, 1H), 2.99-2.91 (m, 3H), 2.70 (br s, 2H), 2.61-2.52 (m, 2H), 1.96-1.86 (m, 2H), 1.85-1.71 (m, 4H), 1.70-1.64 (m, 2H), 1.59 (br dd, J=7.2, 12.4 Hz, 2H), 1.55-1.47 (m, 2H); LCMS (ESI, M+1): m/z=600.3.

### Example 20

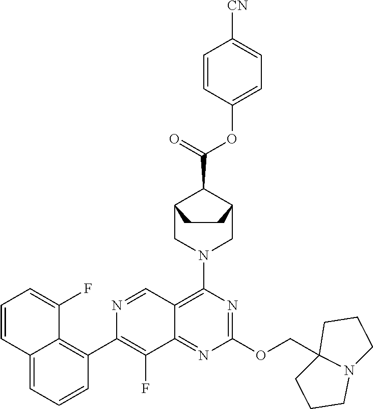 [SMILES: N#Cc1ccc(OC(=O)[C@@H]2[C@@H]3CC[C@H]2CN(c2nc(OCC45CCCN4CCC5)nc4c(F)c(-c5cccc6cccc(F)c56)ncc24)C3)cc1]

### 4-cyanophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

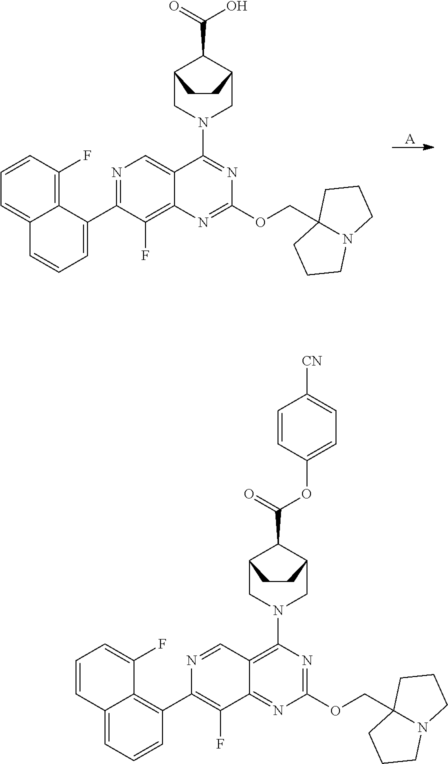

Step A. 4-cyanophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (50 mg, 1.0 equiv), 4-hydroxybenzonitrile (30.5 mg, 3.0 equiv) and DMAP (15.6 mg, 1.5 equiv) in DCM (1.00 mL) was added EDCI (24.6 mg, 1.5 equiv). The reaction was stirred at 10 °C for 16 hours. The mixture was concentrated and purified with prep-HPLC [column: Phenomenex Luna C18 150×25 mm×10 μm; A: water (TFA), B: ACN, B %: 30%-60% over 9 min] to afford the title compound (25.5 mg, 42% yield) as white solid; ¹H NMR (400 MHz, METHANOL-d4) (400 MHz, DMSO-d6) δ=9.15 (s, 1H), 8.18 (br d, J=8.4 Hz, 1H), 7.99-7.90 (m, 3H), 7.78-7.70 (m, 1H), 7.66-7.62 (m, 1H), 7.61-7.55 (m, 1H), 7.45-7.39 (m, 2H), 7.31 (dd, J=7.2, 13.2 Hz, 1H), 4.66 (br d, J=12.0 Hz, 2H), 4.07 (s, 2H), 3.71 (br d, J=12.4 Hz, 2H), 2.93 (td, J=5.2, 10.4 Hz, 2H), 2.87 (br s, 2H), 2.59-2.51 (m, 3H), 1.96-1.86 (m, 2H), 1.85-1.70 (m, 6H), 1.67-1.52 (m, 4H); LCMS (ESI, M+1): m/z=687.3.

### Example 21

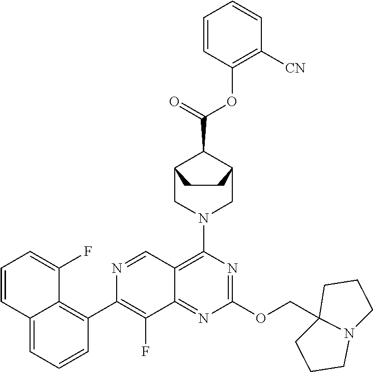 [SMILES: N#Cc1ccccc1OC(=O)[C@@H]1[C@@H]2CC[C@H]1CN(c1nc(OCC34CCCN3CCC4)nc3c(F)c(-c4cccc5cccc(F)c45)ncc13)C2]

### 2-cyanophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

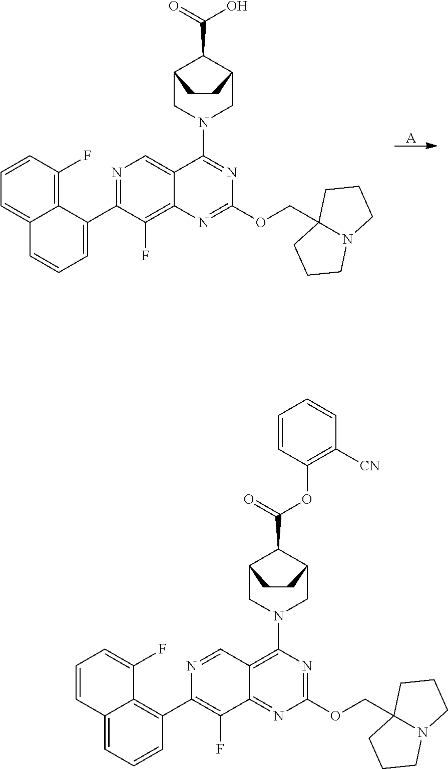

Step A. 2-cyanophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (50.0 mg, 1.0 equiv) and 2-hydroxybenzonitrile (30.5 mg, 3.0 equiv) in DCM (1 mL) were added DMAP (15.6 mg, 1.5 eq) and EDCI (24.5 mg, 1.5 equiv). The reaction was stirred at 30 °C for 2 hours. The mixture was concentrated and purified with prep-HPLC [Phenomenex Luna C18 150×25 mm×10 μm; A: water (TFA); B: ACN; B %: 32%-62% over 9 min], followed by prep-HPLC [Phenomenex luna C18 150×25 mm×10 μm; A: water (FA); B: ACN; B %: 33%-53% over 10 min] to afford the title compound (11.0 mg, 18% yield) as white solid; ¹H NMR (400 MHz, DMSO-d6) δ=9.20 (s, 1H), 8.19 (br d, J=8.8 Hz, 1H), 8.01-7.92 (m, 2H), 7.83 (m, 1H), 7.78-7.71 (t, J=7.6 Hz, 1H), 7.68-7.56 (m, 2H), 7.55-7.46 (m, 2H), 7.32 (dd, J=7.2, 13.2 Hz, 1H), 4.89-4.59 (m, 2H), 4.53-4.32 (m, 2H), 3.78 (br t, J=12.0 Hz, 2H), 3.50-3.36 (m, 3H), 3.13-2.87 (m, 4H), 2.14-2.06 (m, 2H), 2.04-1.81 (m, 8H), 1.68-1.50 (m, 2H); LCMS (ESI, M+1): m/z=687.3.

### Example 22

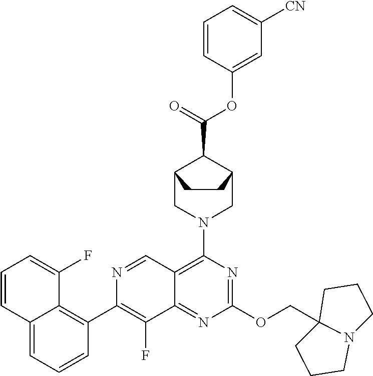 [SMILES: N#Cc1cccc(OC(=O)[C@@H]2[C@@H]3CC[C@H]2CN(c2nc(OCC45CCCN4CCC5)nc4c(F)c(-c5cccc6cccc(F)c56)ncc24)C3)c1]

### 3-cyanophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

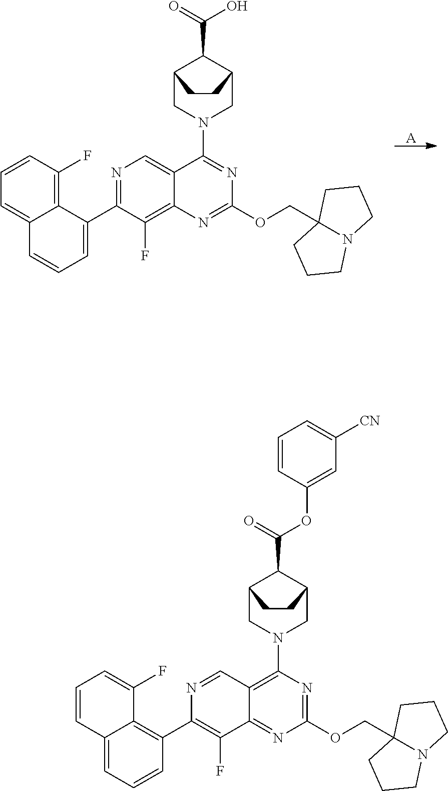

Step A. 3-cyanophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (50.0 mg, 1.0 equiv) and 3-hydroxybenzonitrile (30.5 mg, 3.0 equiv) in DCM (1 mL) were added DMAP (15.6 mg, 1.5 eq) and EDCI (24.5 mg, 1.5 equiv). The reaction was stirred at 30 °C for 2 hours. The mixture was concentrated and purified with prep-HPLC [Phenomenex Luna C18 150×25 mm×10 μm; A: water (TFA); B: ACN; B %: 30%-60% over 9 min] and prep-HPLC [Phenomenex luna C18 150×25 mm×10 μm; A: water (FA); B: ACN; B %: 25%-55% over 10 min], and then followed by prep-HPLC [YMC-Actus Triart C18 150×30 mm×7 μm; A: water (FA); B: ACN; B %: 30%-60% over 10 min] to afford the title compound (14.5 mg, 24% yield) as white solid; ¹H NMR (400 MHz, DMSO-d6) δ=9.15 (s, 1H), 8.18 (br d, J=8.0 Hz, 1H), 7.94 (d, J=8.0 Hz, 1H), 7.84-7.71 (m, 3H), 7.69-7.62 (m, 2H), 7.61-7.53 (m, 2H), 7.32 (dd, J=7.2, 13.2 Hz, 1H), 4.66 (br d, J=10.8 Hz, 2H), 4.10 (s, 2H), 3.72 (br d, J=12.4 Hz, 2H), 3.30 (s, 1H), 3.01-2.92 (m, 2H), 2.87 (br s, 2H), 2.58 (td, J=6.8, 9.8 Hz, 2H), 1.91 (m, 2H), 1.87-1.73 (m, 6H), 1.66-1.55 (m, 4H); LCMS (ESI, M+1): m/z=687.3.

### Example 23

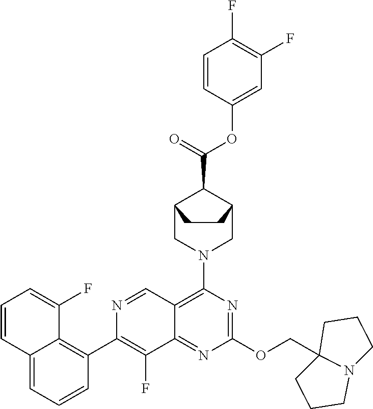 [SMILES: O=C(Oc1ccc(F)c(F)c1)[C@@H]1[C@@H]2CC[C@H]1CN(c1nc(OCC34CCCN3CCC4)nc3c(F)c(-c4cccc5cccc(F)c45)ncc13)C2]

### 3,4-difluorophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

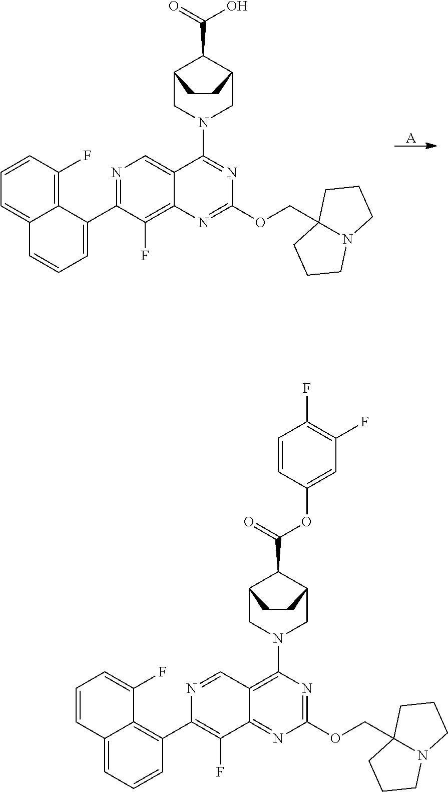

Step A. 3,4-difluorophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (110 mg, 1.0 equiv), 3,4-difluorophenol (48.9 mg, 2.0 equiv) and DMAP (34.4 mg, 1.5 equiv) in DCM (1.00 mL) was added EDCI (54.0 mg, 1.5 equiv). The reaction was stirred at 40 °C for 1 hour. The mixture was diluted with water (20 mL) and extracted with DCM (2×10 mL). The combined organic layers were dried over anhydrous sodium sulfate, concentrated and purified with prep-TLC (Dichloromethane:Methanol=8/1), followed by prep-HPLC [column: YMC Triart C18 150×25 mm×5 μm; A: water (FA), B: ACN, B %: 32%-62% over 2 min]. to afford the title compound (2.90 mg, 2% yield, HCOOH salt) as white solid; ¹H NMR (400 MHz, METHANOL-d4) δ=9.06 (s, 1H), 8.00 (br d, J=7.6 Hz, 1H), 7.75 (d, J=8.4 Hz, 1H), 7.67-7.58 (m, 2H), 7.50-7.42 (m, 1H), 7.24-7.17 (m, 1H), 7.17-7.09 (m, 1H), 7.03-6.95 (m, 1H), 6.88-6.82 (m, 1H), 4.91-4.80 (m, 1H), 4.80-4.71 (m, 1H), 4.28 (br s, 2H), 3.67-3.51 (m, 2H), 3.31-3.14 (m, 2H), 3.04 (s, 1H), 2.97-2.86 (m, 2H), 2.77-2.61 (m, 2H), 2.22-2.10 (m, 2H), 1.99-1.88 (m, 6H), 1.80-1.67 (m, 4H); LCMS (ESI, M+1): m/z=698.3.

### Example 24

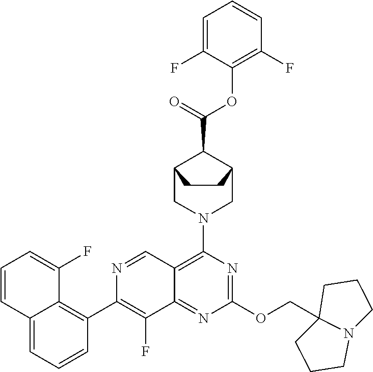 [SMILES: O=C(Oc1c(F)cccc1F)[C@@H]1[C@@H]2CC[C@H]1CN(c1nc(OCC34CCCN3CCC4)nc3c(F)c(-c4cccc5cccc(F)c45)ncc13)C2]

### 2,6-difluorophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

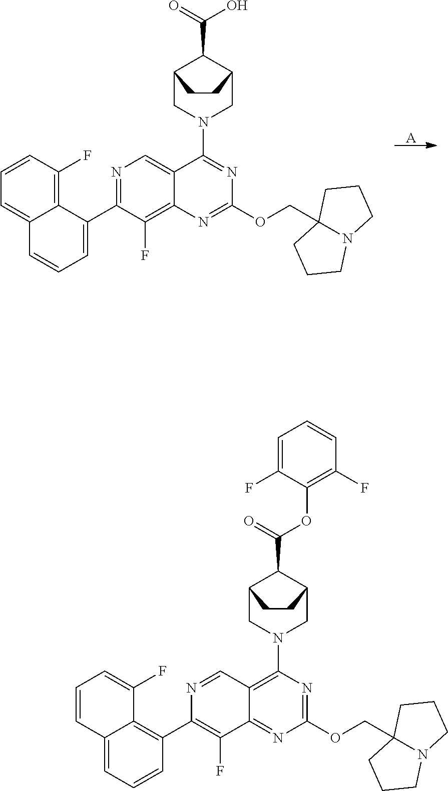

Step A. 2,6-difluorophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (50.0 mg, 1.0 equiv), 2,6-difluorophenol (33.3 mg, 3.0 equiv) and DMAP (15.6 mg, 1.5 equiv) in DCM (1.00 mL) was added EDCI (24.5 mg, 1.5 equiv). The reaction was stirred at 10 °C for 4 hours. The mixture was concentrated and purified with prep-HPLC [column: Phenomenex Luna C18 150×25 mm×10 μm; A: water (TFA), B: ACN, B %: 65% over 9 min] to afford the title compound (8.80 mg, 17% yield) as white solid; ¹H NMR (400 MHz, METHANOL-d4) δ=9.16 (s, 1H), 8.13 (br d, J=8.4 Hz, 1H), 7.87 (d, J=8.4 Hz, 1H), 7.76-7.68 (m, 1H), 7.62 (d, J=6.4 Hz, 1H), 7.54 (dt, J=5.2, 8.0 Hz, 1H), 7.32 (tt, J=6.0, 8.4 Hz, 1H), 7.20 (dd, J=7.6, 13.2 Hz, 1H), 7.15-7.08 (m, 2H), 4.89 (br s, 2H), 4.68 (s, 2H), 3.82 (br d, J=12.4 Hz, 2H), 3.76-3.65 (m, 2H), 3.41 (s, 1H), 3.30-3.26 (m, 2H), 2.98 (br s, 2H), 2.42-2.32 (m, 2H), 2.30-2.07 (m, 6H), 2.03-1.92 (m, 2H), 1.76-1.65 (m, 2H); LCMS (ESI, M+1): m/z=698.5.

### Example 25

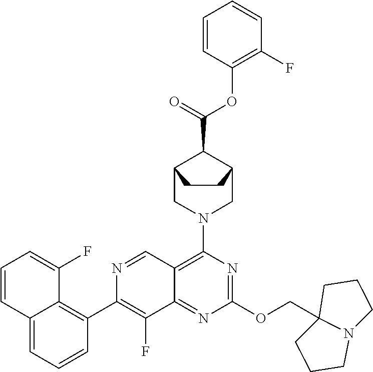 [SMILES: O=C(Oc1ccccc1F)[C@@H]1[C@@H]2CC[C@H]1CN(c1nc(OCC34CCCN3CCC4)nc3c(F)c(-c4cccc5cccc(F)c45)ncc13)C2]

### 2-fluorophenyl (1R,5S,8s)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

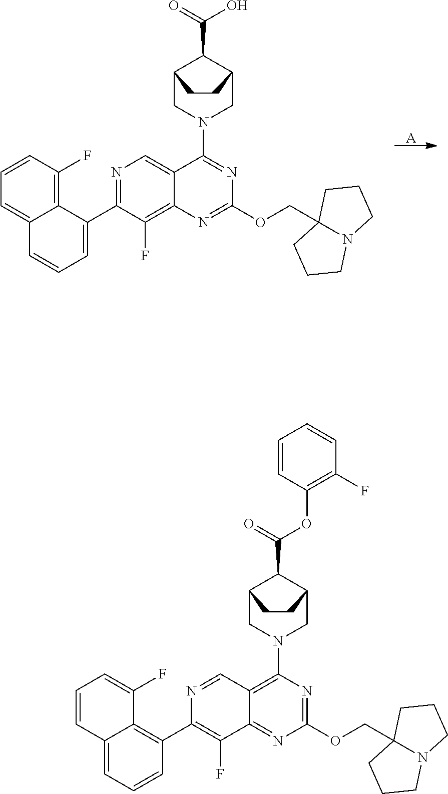

Step A. 2-fluorophenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (50.0 mg, 1.0 equiv), 2-fluorophenol (28.7 mg, 3.0 equiv) and DMAP (15.6 mg, 1.5 equiv) in DCM (1.00 mL) was added EDCI (24.5 mg, 1.5 equiv). The reaction was stirred at 10 °C for 4 hours. The mixture was concentrated and purified with prep-HPLC [column: Phenomenex Luna C18 150×25 mm×10 μm; A: water (TFA), B: ACN, B %: 36%-66% over 9 min] to afford the title compound (8.88 mg, 17% yield) as white solid; ¹H NMR (400 MHz, METHANOL-d4) δ=9.16 (s, 1H), 8.13 (d, J=8.4 Hz, 1H), 7.87 (d, J=8.0 Hz, 1H), 7.75-7.68 (m, 1H), 7.62 (dd, J=1.2, 7.2 Hz, 1H), 7.54 (dt, J=5.2, 8.0 Hz, 1H), 7.33-7.23 (m, 2H), 7.23-7.17 (m, 3H), 4.91-4.86 (m, 2H), 4.68 (s, 2H), 3.81 (br d, J=12.8 Hz, 2H), 3.76-3.66 (m, 2H), 3.34 (s, 1H), 3.30-3.26 (m, 2H), 2.97 (br s, 2H), 2.41-2.31 (m, 2H), 2.29-2.17 (m, 3H), 2.17-2.07 (m, 3H), 2.02-1.94 (m, 2H), 1.73-1.65 (m, 2H); LCMS (ESI, M+1): m/z=680.5.

### Example 26

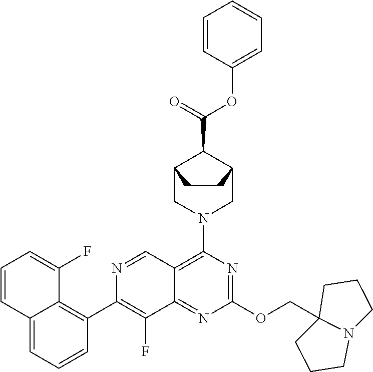 [SMILES: O=C(Oc1ccccc1)[C@@H]1[C@@H]2CC[C@H]1CN(c1nc(OCC34CCCN3CCC4)nc3c(F)c(-c4cccc5cccc(F)c45)ncc13)C2]

### phenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate

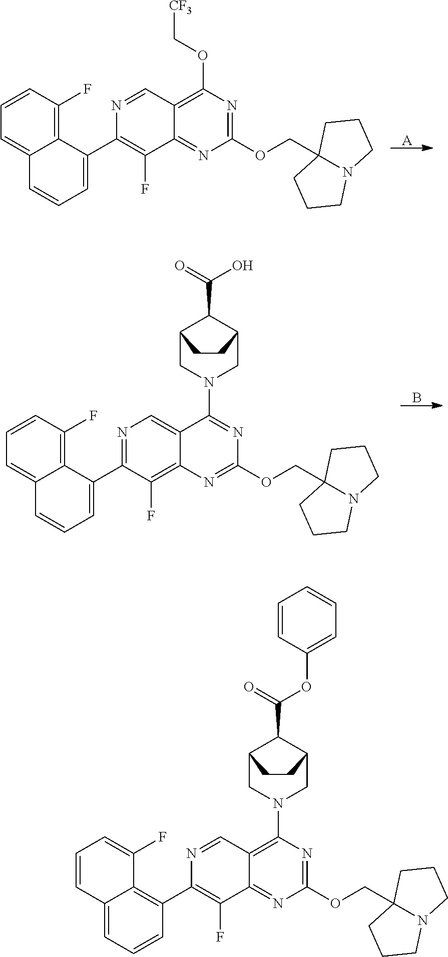

Step A. (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid: To a mixture of (1R,5S,8r)-3-azabicyclo[3.2.1]octane-8-carboxylic acid (1.01 g, 2.0 equiv, HCl), K₃PO₄ (1.68 g, 3.0 equiv) and 4 Å molecular sieve (50.0 mg) in DMF (20.0 mL) was added 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (1.40 g, 1.0 equiv). The reaction was stirred at 60 °C for 2 hours. The mixture was filtered and the filtrate was purified with reversed phase flash chromatography [C18, 0.1% NH₃·H₂O condition] to afford the title compound (1.1 g, 69% yield) as brown solid; LCMS (ESI, M+1): m/z=586.4.

Step B. phenyl (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylate: To a solution of (1R,5S,8r)-3-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-azabicyclo[3.2.1]octane-8-carboxylic acid (50.0 mg, 1.0 equiv), phenol (24.1 mg, 3.0 equiv) and DMAP (15.6 mg, 1.5 equiv) in DCM (1.00 mL) was added EDCI (24.5 mg, 1.5 equiv). The reaction was stirred at 10 °C for 4 hours. The mixture was concentrated and purified with prep-HPLC [column: Phenomenex Luna C18 150×25 mm×10 μm; A: water (TFA), B: ACN, B %: 35%-65% over 9 min] to afford the title compound (10.0 mg, 18% yield) as white solid; ¹H NMR (400 MHz, METHANOL-d4) δ=9.15 (s, 1H), 8.13 (br d, J=8.4 Hz, 1H), 7.86 (d, J=8.4 Hz, 1H), 7.74-7.68 (m, 1H), 7.62 (d, J=6.4 Hz, 1H), 7.54 (dt, J=5.2, 8.0 Hz, 1H), 7.45-7.38 (m, 2H), 7.29-7.24 (m, 1H), 7.20 (dd, J=7.6, 12.8 Hz, 1H), 7.10 (d, J=8.0 Hz, 2H), 4.90-4.86 (m, 2H), 4.62 (s, 2H), 3.79 (br d, J=12.8 Hz, 2H), 3.66-3.56 (m, 2H), 3.28 (s, 1H), 3.25-3.17 (m, 2H), 2.95 (br s, 2H), 2.37-2.27 (m, 2H), 2.25-2.01 (m, 6H), 1.99-1.90 (m, 2H), 1.73-1.63 (m, 2H); LCMS (ESI, M+1): m/z=662.6.

### Example 27

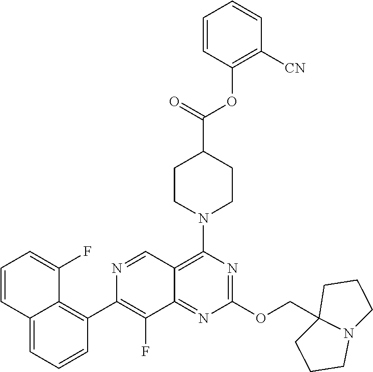 [SMILES: N#Cc1ccccc1OC(=O)C1CCN(c2nc(OCC34CCCN3CCC4)nc3c(F)c(-c4cccc5cccc(F)c45)ncc23)CC1]

### 2-cyanophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

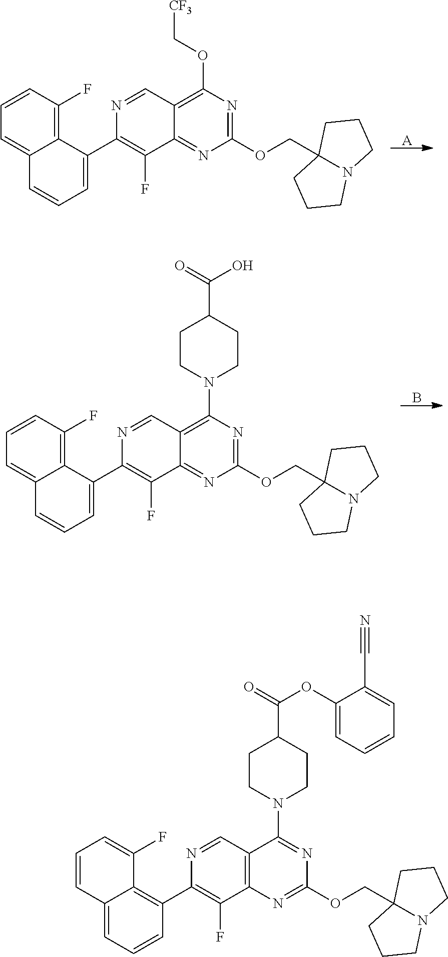

Step A. 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.0 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as a white solid; LCMS (ESI, M+1): m/z=560.3.

Step B. 2-cyanophenyl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a mixture of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and 2-hydroxybenzonitrile (31.9 mg, 3.0 equiv) in DCM (2.00 mL) were added DMAP (16.4 mg, 1.5 equiv) and EDCI (25.7 mg, 1.5 equiv). The reaction was stirred at 25 °C for 4 hours. The mixture was concentrated and purified with prep-HPLC [column: Phenomenex luna C18 150×25 mm×10 μm; mobile phase: [water (FA)-ACN]; gradient: 37%-67% B over 9 min] to afford the title compound (9.96 mg, 14.6% yield) as off-white solid; ¹H NMR (400 MHz, DMSO-d₆) δ=10.26 (br s, 1H), 9.19 (s, 1H), 8.20 (br d, J=8.4 Hz, 1H), 7.96 (t, J=8.4 Hz, 2H), 7.83 (t, J=8.0 Hz, 1H), 7.76 (t, J=7.6 Hz, 1H), 7.65 (d, J=7.2 Hz, 1H), 7.60 (br d, J=5.2 Hz, 1H), 7.54-7.49 (m, 2H), 7.32 (dd, J=7.6, 13.1 Hz, 1H), 4.58 (s, 4H), 3.73-3.64 (m, 2H), 3.52 (br dd, J=5.6, 11.6 Hz, 2H), 3.26-3.18 (m, 2H), 2.33-2.27 (m, 2H), 2.19 (br dd, J=5.6, 11.6 Hz, 2H), 2.10 (br d, J=6.4 Hz, 2H), 2.07-1.94 (m, 7H); LCMS (ESI, M+1): m/z=661.3.

### Example 28

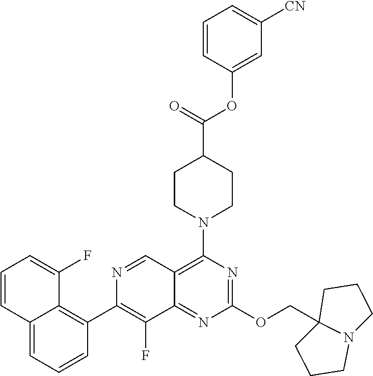 [SMILES: N#Cc1cccc(OC(=O)C2CCN(c3nc(OCC45CCCN4CCC5)nc4c(F)c(-c5cccc6cccc(F)c56)ncc34)CC2)c1]

### 3-cyanophenyl 1-(8-fluoro-7-(8-fluoronaphthalen-1-yl)-2-((tetrahydro-1H-pyrrolizin-7a(5H)-yl) methoxy)pyrido[4,3-d]pyrimidin-4-yl)piperidine-4-carboxylate

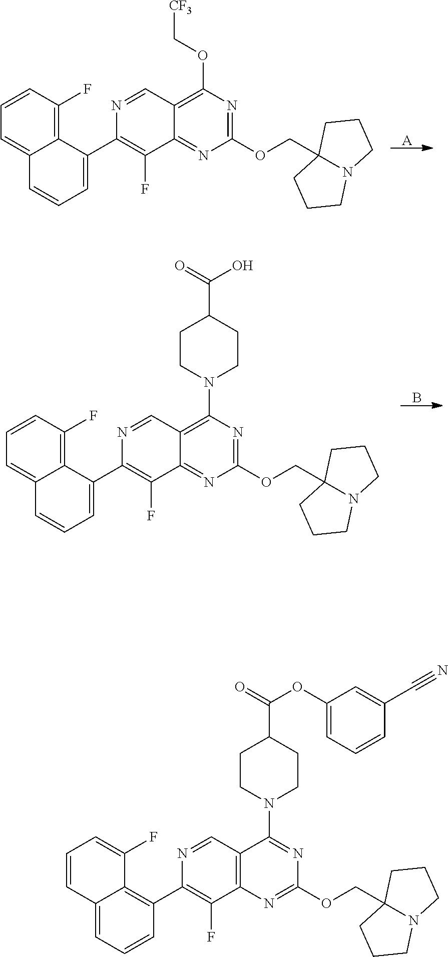

Step A. 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.00 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as a white solid; LCMS (ESI, M+1): m/z=560.3.

Step B. 3-cyanophenyl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a mixture of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and 3-hydroxybenzonitrile (31.9 mg, 3.0 equiv) in DCM (2.00 mL) were added DMAP (16.4 mg, 1.5 equiv) and EDCI (25.7 mg, 1.5 equiv). The reaction was stirred at 25 °C for 4 hours. The mixture was concentrated and purified by prep-HPLC [column: Phenomenex luna C18 150×25 mm×10 μm; mobile phase: [water (FA)-ACN]; gradient: 25%-55% B over 10 min] and purified by prep-HPLC [column: Phenomenex luna C18 150×25 mm×10 μm; mobile phase: [water (FA)-ACN]; gradient: 37%-67% B over 9 min] to afford the title compound (7.3 mg, 11% yield) as a yellow solid; ¹H NMR (400 MHz, DMSO-d₆) δ=10.23 (br s, 1H), 9.18 (s, 1H), 8.20 (br d, J=8.4 Hz, 1H), 7.95 (d, J=8.0 Hz, 1H), 7.81-7.73 (m, 3H), 7.70-7.63 (m, 2H), 7.62-7.54 (m, 2H), 7.32 (s, 1H), 4.57 (s, 2H), 3.28-3.16 (m, 4H), 2.27-1.97 (m, 13H), 1.23 (br s, 2H), 1.05 (t, J=7.2 Hz, 2H); LCMS (ESI, M+1): m/z=661.4.

### Example 29

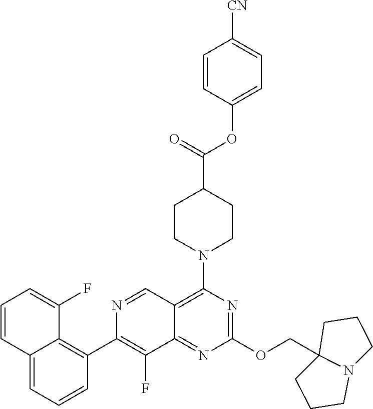 [SMILES: N#Cc1ccc(OC(=O)C2CCN(c3nc(OCC45CCCN4CCC5)nc4c(F)c(-c5cccc6cccc(F)c56)ncc34)CC2)cc1]

### 4-cyanophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

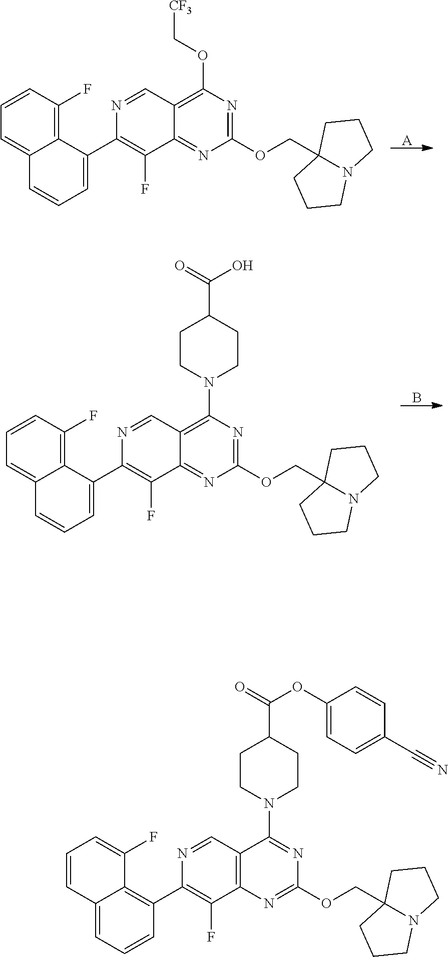

Step A 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.0 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as white solid; LCMS (ESI, M+1): m/z=560.3

Step B. 4-cyanophenyl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a mixture of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and 4-hydroxybenzonitrile (31.9 mg, 3.0 equiv) in DCM (2.00 mL) were added DMAP (16.4 mg, 1.5 equiv) and EDCI (25.7 mg, 1.5 equiv). The reaction was stirred at 25 °C for 4 hours. The mixture was concentrated and purified with prep-HPLC [column: Phenomenex luna C18 150×25 mm×10 μm; mobile phase: [water (FA)-ACN]; gradient: 36%-66% B over 9 min] to afford the title compound (20.3 mg, 33% yield) as off-white solid; ¹H NMR (400 MHz, DMSO-d₆) δ=10.24 (br s, 1H), 9.18 (s, 1H), 8.20 (br d, J=8.8 Hz, 1H), 7.98-7.93 (m, 3H), 7.75 (d, J=8.0 Hz, 1H), 7.66-7.57 (m, 2H), 7.42 (d, J=8.4 Hz, 2H), 7.32 (dd, J=7.6, 13.1 Hz, 1H), 4.60-4.53 (m, 4H), 3.69-3.45 (m, 4H), 2.33-2.22 (m, 3H), 2.18 (br dd, J=5.6, 11.4 Hz, 2H), 2.10 (br d, J=6.4 Hz, 2H), 2.07-1.93 (m, 8H); LCMS (ESI, M+1): m/z=661.3.

### Example 30

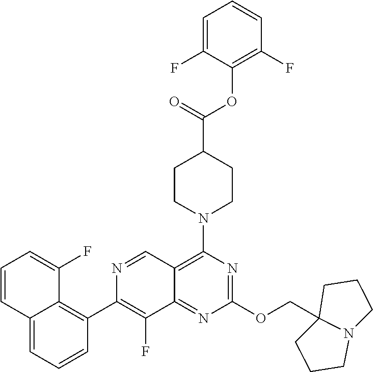 [SMILES: O=C(Oc1c(F)cccc1F)C1CCN(c2nc(OCC34CCCN3CCC4)nc3c(F)c(-c4cccc5cccc(F)c45)ncc23)CC1]

### 2,6-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

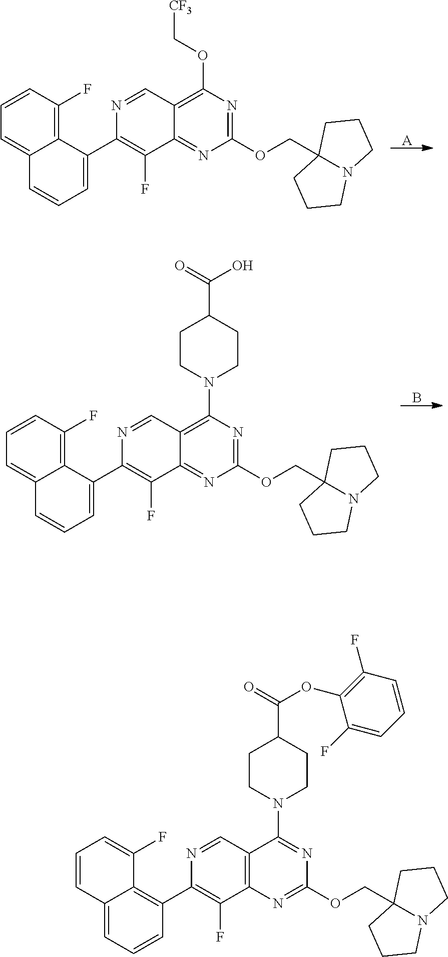

Step A. 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.0 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as white solid; LCMS (ESI, M+1): m/z=560.3.

Step B. 2,6-difluorophenyl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a mixture of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and 2,6-difluorophenol (34.8 mg, 3.0 equiv) in DCM (2.0 mL) were added DMAP (16.4 mg, 1.5 equiv) and EDCI (25.7 mg, 1.5 equiv). The reaction was stirred at 25 °C for 4 hours. The reaction mixture was concentrated and purified with prep-HPLC [column: Phenomenex luna C18 150×25 mm×10 μm; mobile phase: [water (FA)-ACN]; gradient: 27%-57% B over 10 min] and prep-HPLC [column: Phenomenex luna C18 150×40 mm×15 μm; mobile phase: [water (TFA)-ACN]; gradient: 35%-65% B over 10 min] to afford the title compound (5.6 mg, 9% yield) as brown solid; ¹H NMR (400 MHz, DMSO-d₆) δ=10.21 (br s, 1H), 9.19 (s, 1H), 8.20 (br d, J=8.4 Hz, 1H), 7.95 (d, J=8.0 Hz, 1H), 7.76 (t, J=7.6 Hz, 1H), 7.67-7.56 (m, 2H), 7.45-7.37 (m, 1H), 7.36-7.28 (m, 3H), 4.61-4.51 (m, 4H), 3.43 (s, 2H), 3.22 (br dd, J=4.4, 11.2 Hz, 2H), 2.34-2.23 (m, 3H), 2.18 (br dd, J=6.0, 11.6 Hz, 2H), 2.15-2.08 (m, 2H), 2.04-1.95 (m, 6H), 1.07-1.03 (m, 2H); LCMS (ESI, M+1): m/z=672.3.

### Example 31

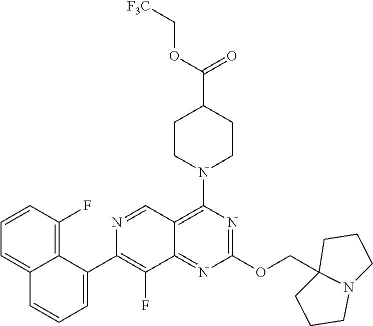 [SMILES: O=C(OCC(F)(F)F)C1CCN(c2nc(OCC34CCCN3CCC4)nc3c(F)c(-c4cccc5cccc(F)c45)ncc23)CC1]

### 2,2,2-trifluoroethyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

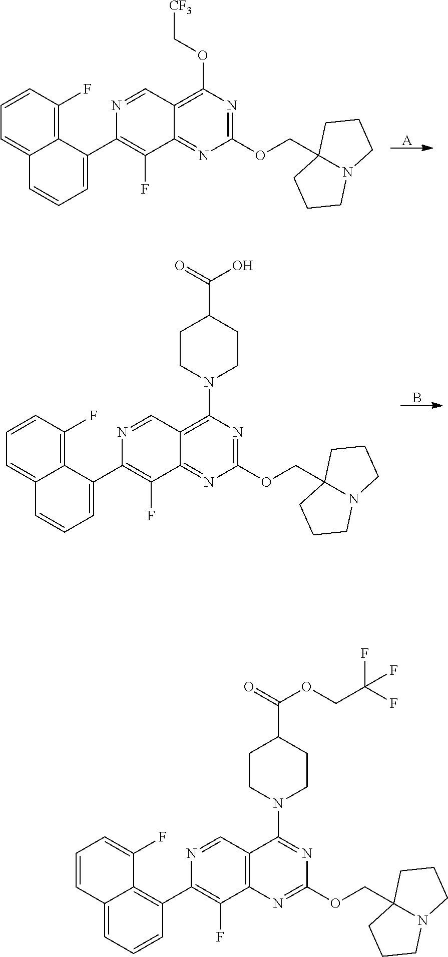

Step A. 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.00 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as white solid; LCMS (ESI, M+1): m/z=560.3.

Step B. 2,2,2-trifluoroethyl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a mixture of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and 2,2,2-trifluoroethanol (26.8 mg, 3.0 equiv) in DCM (2.0 mL) were added DMAP (16.37 mg, 1.5 equiv) and EDCI (25.69 mg, 1.5 equiv). The reaction was stirred at 25 °C for 4 hours. The reaction mixture was concentrated and purified by prep-HPLC [column: Phenomenex luna C18 150×25 mm×10 μm; mobile phase: [water (FA)-ACN]; gradient: 24%-54% B over 10 min] to afford the title compound (16.7 mg, 28% yield) as white solid; ¹H NMR (400 MHz, DMSO-d₆) δ=9.09 (s, 1H), 8.25 (s, 1H), 8.18 (br d, J=8.4 Hz, 1H), 7.93 (d, J=8.4 Hz, 1H), 7.79-7.70 (m, 1H), 7.66-7.54 (m, 2H), 7.31 (dd, J=7.6, 13.2 Hz, 1H), 4.80 (q, J=9.2 Hz, 2H), 4.46 (br d, J=13.2 Hz, 2H), 4.08 (s, 2H), 3.52 (br t, J=12.0 Hz, 2H), 2.97 (br s, 3H), 2.61-2.52 (m, 2H), 2.17-2.03 (m, 2H), 1.95-1.73 (m, 8H), 1.65-1.54 (m, 2H); LCMS (ESI, M+1): m/z=642.3.

### Example 32

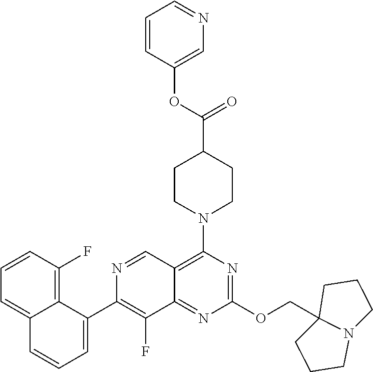 [SMILES: O=C(Oc1cccnc1)C1CCN(c2nc(OCC34CCCN3CCC4)nc3c(F)c(-c4cccc5cccc(F)c45)ncc23)CC1]

### pyridin-3-yl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

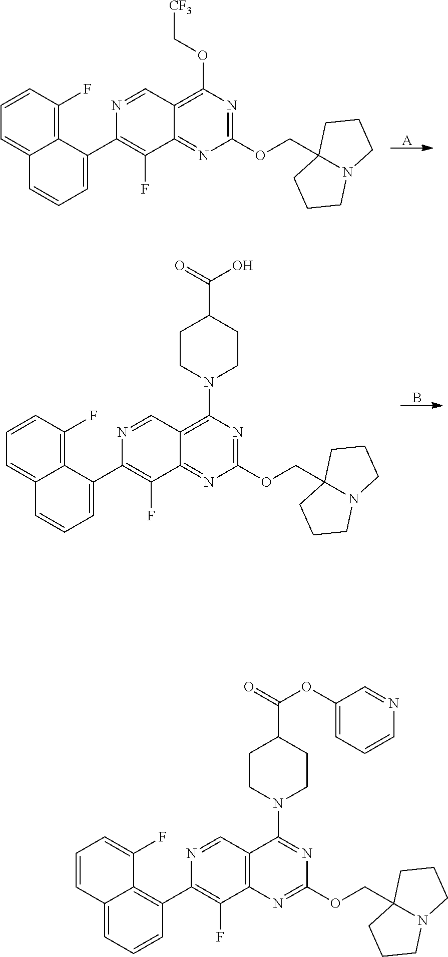

Step A. 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.0 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as white solid; LCMS (ESI, M+1): m/z=560.3

Step B. pyridin-3-yl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a mixture of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and pyridin-3-ol (25.49 mg, 3.0 equiv) in DCM (2.0 mL) were added DMAP (16.4 mg, 1.5 equiv) and EDCI (25.7 mg, 1.5 equiv). The reaction was stirred at 25 °C for 4 hours. The reaction mixture was concentrated and purified by prep-HPLC [column: Phenomenex luna C18 150×25 mm×10 μm; mobile phase: [water (FA)-ACN]; gradient: 28%-58% B over 10 min] to afford the title compound (27.4 mg, 47% yield) as white solid; ¹H NMR (400 MHz, DMSO-d₆) δ=9.19 (s, 1H), 8.55-8.45 (m, 2H), 8.20 (br d, J=8.4 Hz, 1H), 7.95 (d, J=8.4 Hz, 1H), 7.79-7.73 (m, 1H), 7.72-7.56 (m, 4H), 7.52 (dd, J=4.8, 8.4 Hz, 1H), 7.32 (dd, J=7.6, 13.2 Hz, 1H), 4.60-4.54 (m, 4H), 3.66 (br d, J=13.2 Hz, 2H), 3.52 (br d, J=5.6 Hz, 2H), 2.27 (br d, J=11.0 Hz, 3H), 2.18 (br dd, J=5.6, 11.2 Hz, 2H), 2.10 (br d, J=6.4 Hz, 2H), 2.06-1.97 (m, 7H); LCMS (ESI, M+1): m/z=637.3.

### Example 33

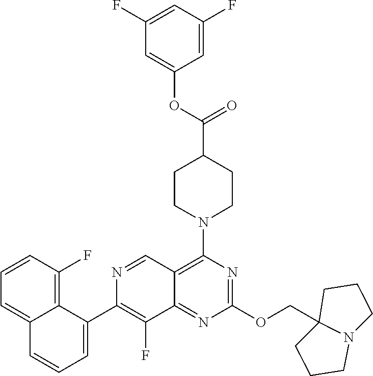 [SMILES: O=C(Oc1cc(F)cc(F)c1)C1CCN(c2nc(OCC34CCCN3CCC4)nc3c(F)c(-c4cccc5cccc(F)c45)ncc23)CC1]

### 3,5-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

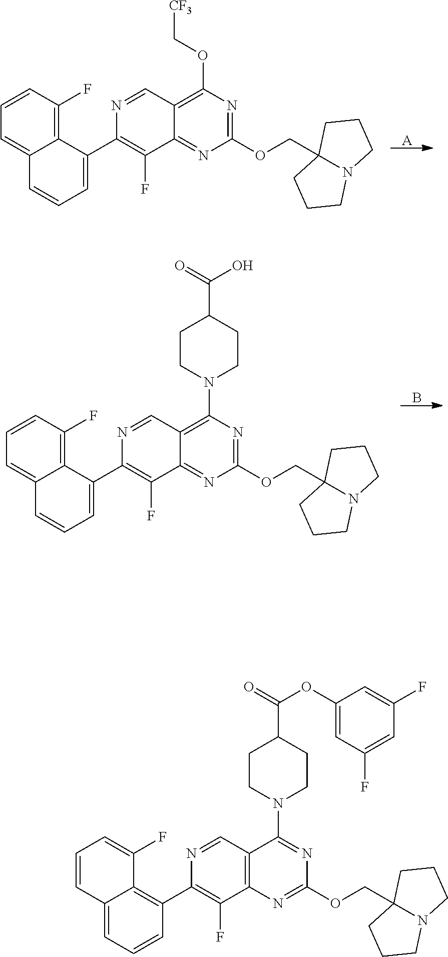

Step A. 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.0 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as white solid; LCMS (ESI, M+1): m/z=560.3

Step B. 3,5-difluorophenyl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a solution of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and 3,5-difluorophenol (34.9 mg, 3.0 equiv) in DCM (2.00 mL) were added DMAP (16.37 mg, 1.5 equiv) and EDCI (25.69 mg, 1.5 equiv). The reaction was stirred at 25 °C for 4 hours The mixture was concentrated and purified by prep-HPLC [column: Phenomenex luna C18 150×25 mm×10 μm; mobile phase: [water (FA)-ACN]; gradient: 30%-60% B over 10 min] to afford the title compound (8.30 mg, 14% yield) as yellow solid; ¹H NMR (400 MHz, DMSO-d₆) δ=9.11 (s, 1H), 8.31 (s, 1H), 8.18 (br d, J=8.0 Hz, 1H), 7.94 (d, J=8.4 Hz, 1H), 7.78-7.70 (m, 1H), 7.65-7.56 (m, 2H), 7.31 (dd, J=7.6, 13.2 Hz, 1H), 7.26-7.18 (m, 1H), 7.09 (dd, J=2.0, 7.6 Hz, 2H), 4.49 (br d, J=13.2 Hz, 2H), 4.08 (s, 2H), 3.64-3.57 (m, 2H), 3.19-3.10 (m, 2H), 2.96-2.91 (m, 2H), 2.21 (br dd, J=3.2, 13.2 Hz, 2H), 2.05-1.97 (m, 2H), 1.93-1.87 (m, 2H), 1.78 (td, J=6.0, 12.0 Hz, 4H), 1.59 (br dd, J=7.6, 12.0 Hz, 2H), 1.23 (br s, 1H); LCMS (ESI, M+1): m/z=672.3.

### Example 34

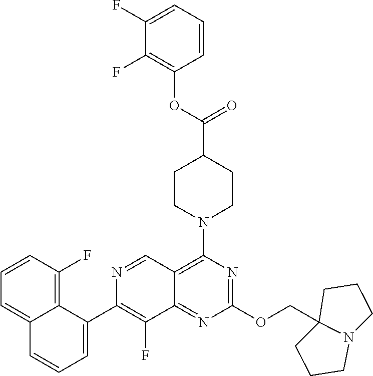 [SMILES: O=C(Oc1cccc(F)c1F)C1CCN(c2nc(OCC34CCCN3CCC4)nc3c(F)c(-c4cccc5cccc(F)c45)ncc23)CC1]

### 2,3-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

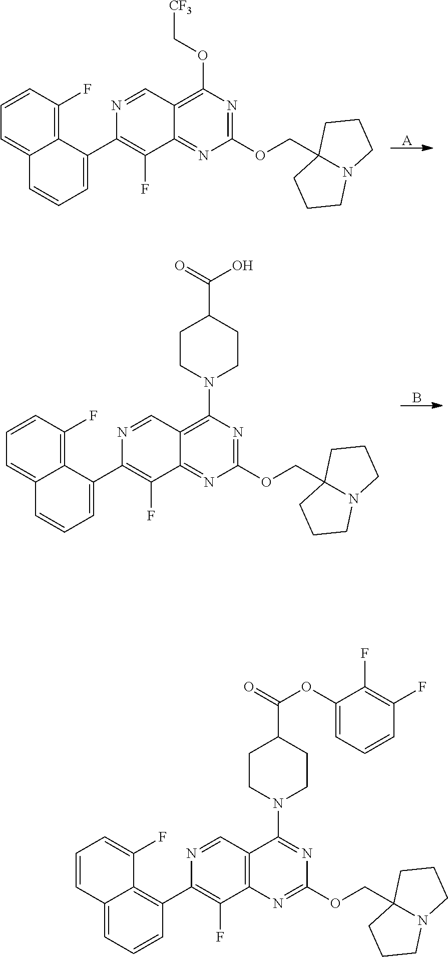

Step A. 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid: To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (300 mg, 1.0 equiv) and 4 Å molecular sieve (100 mg) in DMF (3.00 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as white solid; LCMS (ESI, M+1): m/z=560.3.

Step B. 2,3-difluorophenyl 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylate: To a solution of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50 mg, 1.0 equiv) and 2,3-difluorophenol (34.8 mg, 3.0 equiv) in dichloromethane (2.00 mL) were added 4-dimethylaminopyridine (16.4 mg, 1.5 equiv) and EDCI (25.7 mg, 1.5 equiv). The mixture was stirred at 25 °C for 4 hours. The reaction mixture was concentrated and purified with prep-HPLC [column: Phenomenex luna C18 150×25 mm×10 μm; mobile phase: [water (FA)-ACN]; gradient: 28%-58% B over 10 min] to afford the title compound (14.3 mg, 23% yield) as yellow solid; ¹H NMR (400 MHz, DMSO-d₆) δ=9.12 (s, 1H), 8.29 (s, 1H), 8.18 (br d, J=8.4 Hz, 1H), 7.93 (d, J=8.0 Hz, 1H), 7.78-7.71 (m, 1H), 7.66-7.56 (m, 2H), 7.48-7.36 (m, 1H), 7.35-7.26 (m, 2H), 7.22 (br dd, J=1.6, 6.8 Hz, 1H), 4.50 (br d, J=13.6 Hz, 2H), 4.08 (s, 2H), 3.64-3.56 (m, 2H), 3.26-3.21 (m, 1H), 2.97-2.91 (m, 2H), 2.24 (br d, J=10.4 Hz, 2H), 2.09-1.72 (m, 10H), 1.59 (br dd, J=7.6, 12.0 Hz, 2H); LCMS (ESI, M+1): m/z=672.3.

### Example 35

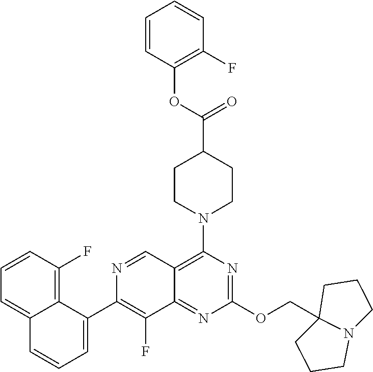 [SMILES: O=C(Oc1ccccc1F)C1CCN(c2nc(OCC34CCCN3CCC4)nc3c(F)c(-c4cccc5cccc(F)c45)ncc23)CC1]

### 2-fluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

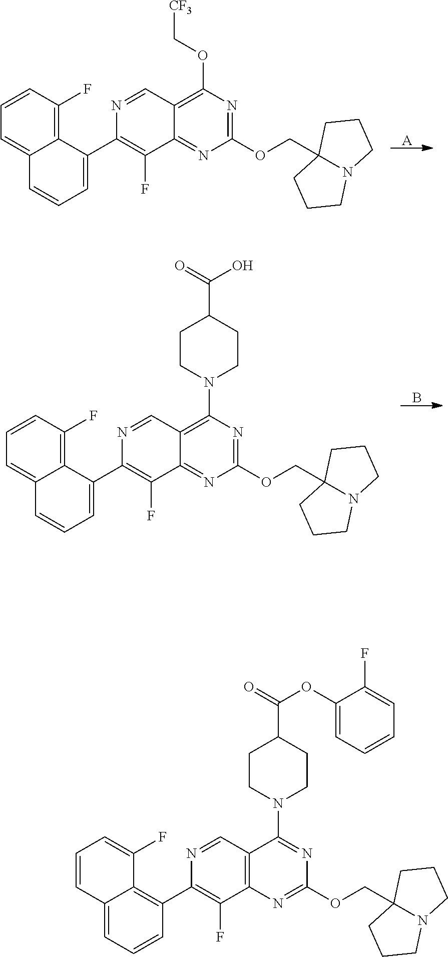

Step A. 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid: To a mixture of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (300 mg, 1.0 equiv) and 4 Å molecular sieve (100 mg) in DMF (3.00 mL) were added K₃PO₄ (600 mg, 5.0 equiv) and piperidine-4-carboxylic acid (281 mg, 3.0 equiv, HCl). The reaction was stirred at 60 °C for 1.5 hours. The mixture was diluted with H₂O (100 mL) and extracted with dichloromethane (3×100 mL). The combined organic layers were dried over Na₂SO₄, concentrated and purified by prep-TLC [petroleum ether/ethyl acetate=3/1] to afford the title compound (250 mg, 72% yield) as white solid; LCMS (ESI, M+1): m/z=560.3.

Step B. 2-fluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 1-(8-fluoro-7-(8-fluoronaphthalen-1-yl)-2-((hexahydro-1H-pyrrolizin-7a-yl)methoxy)pyrido[4,3-d]pyrimidin-4-yl)piperidine-4-carboxylic acid (50.0 mg, 1.0 equiv) and 2-fluorophenol (30.0 mg, 3.0 equiv) in dichloromethane (2.0 mL) were added DMAP (16.4 mg, 1.5 equiv) and EDCI (25.7 mg, 1.5 equiv). The reaction was stirred at 25 °C for 4 hours. The mixture was concentrated and purified by prep-HPLC [column: Phenomenex luna C18 250×50 mm×15 μm; mobile phase: [water (FA)-ACN]; gradient: 38%-68% B over 9 min] to afford the title compound (11.2 mg, 19% yield) as white solid; ¹H NMR (400 MHz, DMSO-d6) δ=9.12 (s, 1H), 8.35 (s, 1H), 8.18 (br d, J=8.2 Hz, 1H), 7.94 (d, J=8.0 Hz, 1H), 7.77-7.72 (m, 1H), 7.64 (dd, J=0.8, 7.2 Hz, 1H), 7.59 (br dd, J=2.8, 8.0 Hz, 1H), 7.40-7.26 (m, 5H), 4.49 (br d, J=13.6 Hz, 2H), 4.08 (s, 2H), 3.64-3.58 (m, 2H), 3.21 (br s, 2H), 2.96-2.90 (m, 2H), 2.27-2.21 (m, 2H), 1.99 (br d, J=10.8 Hz, 2H), 1.93-1.87 (m, 2H), 1.79 (br dd, J=5.2, 11.2 Hz, 4H), 1.62-1.55 (m, 2H), 1.23 (br s, 1H); LCMS (ESI, M+1): m/z=654.3.

### Example 36

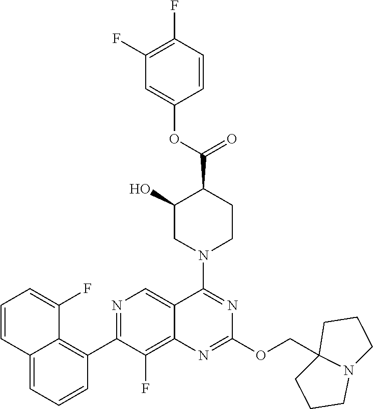 [SMILES: O=C(Oc1ccc(F)c(F)c1)[C@H]1CCN(c2nc(OCC34CCCN3CCC4)nc3c(F)c(-c4cccc5cccc(F)c45)ncc23)C[C@H]1O]

### 3,4-difluorophenyl (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate

### Example 37

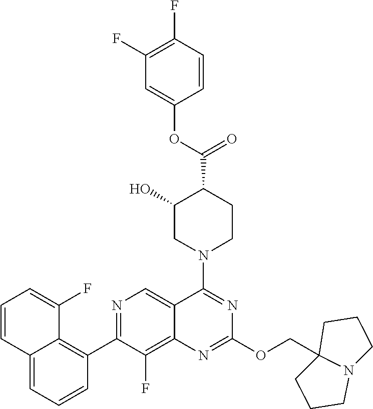 [SMILES: O=C(Oc1ccc(F)c(F)c1)[C@@H]1CCN(c2nc(OCC34CCCN3CCC4)nc3c(F)c(-c4cccc5cccc(F)c45)ncc23)C[C@@H]1O]

### 3,4-difluorophenyl (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate

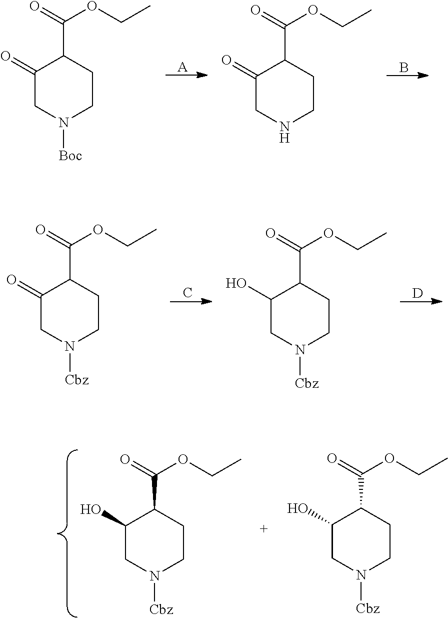

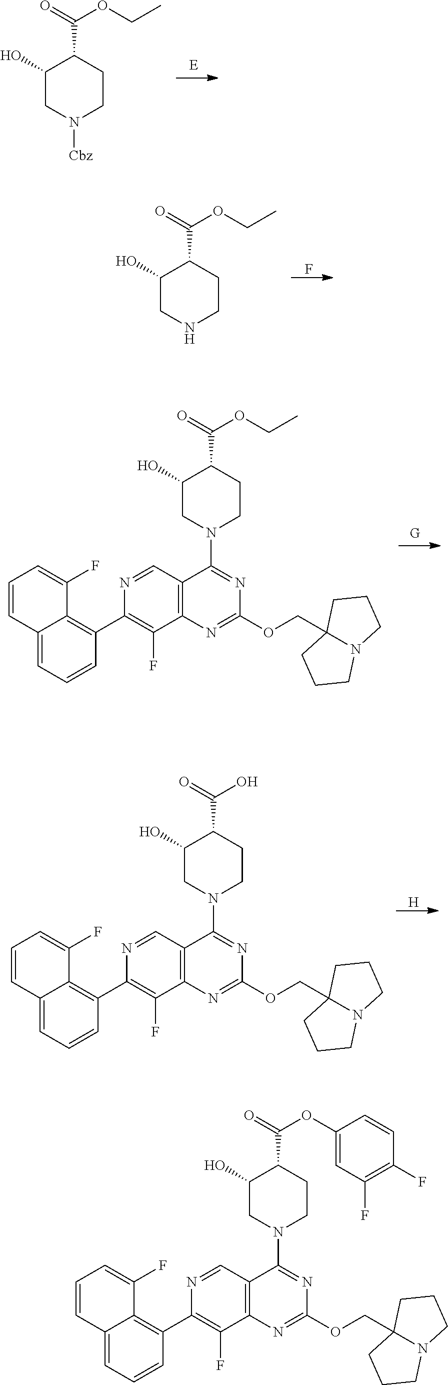

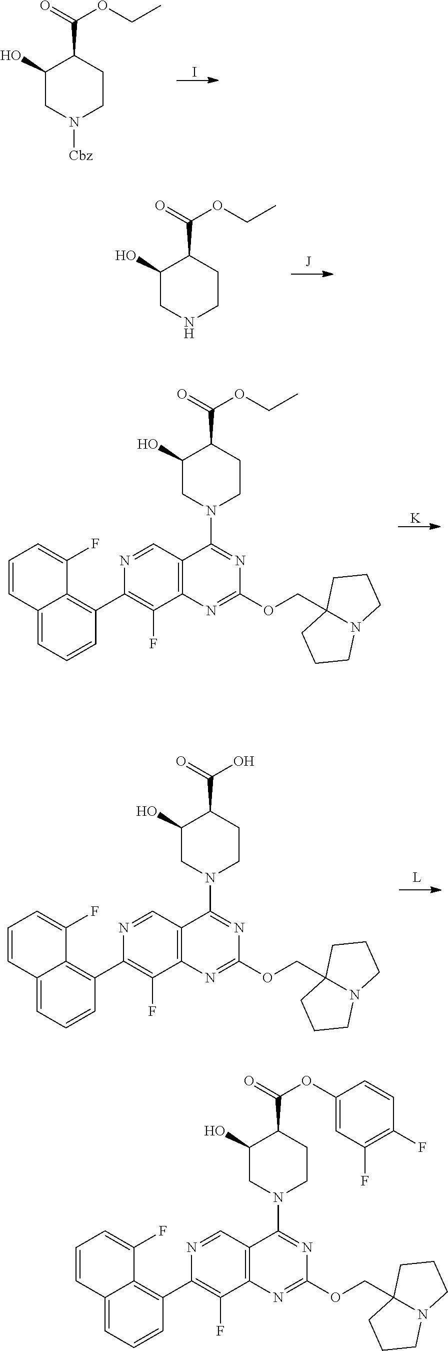

Step A. ethyl 3-oxopiperidine-4-carboxylate: To a solution of 1-(tert-butyl) 4-ethyl 3-oxopiperidine-1,4-dicarboxylate (40.0 g, 1.0 equiv) in MeOH (150 mL) was added HCl·MeOH (4 M, 250 mL). The reaction was stirred at 25 °C for 2 hours. The mixture was concentrated under reduced pressure to afford the title compound. (40.0 g, crude) as a yellow solid; LCMS (ESI, M+1): m/z=172.3.

Step B. 1-benzyl 4-ethyl 3-oxopiperidine-1,4-dicarboxylate: To a mixture of ethyl 3-oxopiperidine-4-carboxylate (30.0 g, 1.0 equiv) and CbzCl (35.9 g, 1.2 equiv) in DCM (300 mL) were added TEA (88.7 g, 5.0 equiv) at 0 °C. The reaction was stirred at 20 °C for 2 hours. The mixture was diluted with water (500 mL) and extracted with ethyl acetate (3×500 mL). The combined organic layers were washed with brine (500 mL), dried over Na₂SO₄, concentrated and purified with column chromatography [SiO₂, petroleum ether/ethyl acetate=20/1 to 5/1] to afford the title compound (23.0 g, 43% yield) as colorless oil; ¹H NMR (400 MHz, CHLOROFORM-d) δ=12.09 (s, 1H), 7.39-7.32 (m, 5H), 5.16 (s, 2H), 4.24 (q, J=7.2 Hz, 2H), 4.12 (br s, 2H), 3.58 (t, J=5.6 Hz, 2H), 2.36 (br s, 2H), 1.31 (t, J=7.2 Hz, 3H).

Step C. 1-benzyl 4-ethyl 3-hydroxypiperidine-1,4-dicarboxylate: To a solution of 1-benzyl 4-ethyl 3-oxopiperidine-1,4-dicarboxylate (10.0 g, 1.0 equiv) in THF (100 mL) was added NaBH₄ (1.95 g, 1.6 equiv) at 0 °C. The reaction was stirred at 0 °C for 1 hour. The mixture was diluted with sat. NH₄Cl (100 mL) and extracted with ethyl acetate (3×100 mL). The combined organic layers were washed with brine (100 mL), dried over Na₂SO₄, concentrated and purified by column chromatography [SiO₂, petroleum ether/ethyl acetate=10/1 to 1/1] to afford the title compound (4.60 g, 46% yield) as yellow oil; ¹H NMR (400 MHz, CHLOROFORM-d) δ=7.40-7.28 (m, 5H), 5.14 (br s, 2H), 4.36-4.09 (m, 5H), 3.04 (br s, 1H), 2.97-2.84 (m, 1H), 2.56 (br d, J=10.4 Hz, 1H), 2.21-2.01 (m, 1H), 1.87-1.70 (m, 1H), 1.28 (t, J=7.2 Hz, 3H); LCMS (ESI, M+1): m/z=308.3.

Step D. 1-benzyl 4-ethyl (3R,4R)-3-hydroxypiperidine-1,4-dicarboxylate and 1-benzyl 4-ethyl (3S,4S)-3-hydroxypiperidine-1,4-dicarboxylate: The residue was purified with SFC [column: DAICEL CHIRALPAK IG (250 mm×30 mm, 10 μm); mobile phase: [CO₂-EtOH(0.1% NH₃H₂O)]; B %: 40%, isocratic elution mode] to afford the title compound (1.50 g, 33% yield) and (1.50 g, 33% yield).

Step E. ethyl (3R,4R)-3-hydroxypiperidine-4-carboxylate: To a solution of 1-benzyl 4-ethyl (3R,4R)-3-hydroxypiperidine-1,4-dicarboxylate (700 mg, 1.0 equiv) in ethyl acetate (8.00 mL) was added Pd/C (1.00 g, 10% purity) under N₂ atmosphere. The reaction was degassed and purged with H₂ 3 times. The reaction was stirred at 25 °C for 4 hours under H₂ (50 psi). The mixture was filtered and concentrated to afford the title compound (300 mg, crude) as a yellow solid; ¹H NMR (400 MHz, CHLOROFORM-d) δ=4.18 (q, J=7.2 Hz, 2H), 4.07 (br s, 1H), 3.13-3.04 (m, 2H), 2.80-2.66 (m, 3H), 2.59 (dt, J=2.8, 12.4 Hz, 1H), 2.54-2.47 (m, 1H), 2.02-1.88 (m, 1H), 1.71 (br dd, J=3.6, 13.2 Hz, 1H), 1.27 (t, J=7.2 Hz, 3H).

Step F. ethyl 1 (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate: To a mixture of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (200 mg, 1.0 equiv) and K₃PO₄ (240 mg, 3.0 equiv) in DMF (3.00 mL) were added ethyl (3R,4R)-3-hydroxypiperidine-4-carboxylate (261 mg, 4.0 equiv) and 4 Å molecular sieve (50.0 mg). The reaction was stirred at 60 °C for 2 hours. The mixture was filtered and purified by prep-HPLC [FA condition] to afford the title compound (180 mg, 80% yield) as yellow solid; LCMS (ESI, M+1): m/z=604.3.

Step G. (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylic acid: To a solution of ethyl (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate (90.0 mg, 1.0 equiv) in THF (1.50 mL) was added NaOH (2 M, 224 μL, 3.0 equiv). The reaction was stirred at 25 °C for 3 hours. The mixture was adjusted pH to 6. The mixture was washed with ethyl acetate (2×20.0 mL). The water layer was lyophilized to afford the title compound. (60.0 mg, crude) as an off white solid; LCMS (ESI, M+1): m/z=576.3.

Step H. 3,4-difluorophenyl (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate: To a solution of (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylic acid (50.0 mg, 1.0 equiv) and 3,4-difluorophenol (33.9 mg, 3.0 equiv) in DCM (1.00 mL) were added DMAP (15.9 mg, 1.5 equiv) and EDCI (25.0 mg, 1.5 equiv). The reaction was stirred at 20 °C for 4 hours. The mixture was diluted with water (20.0 mL) and extracted with DCM (2×20 mL). The combined organic layers were washed with brine (20.0 mL), dried over Na₂SO₄, concentrated and purified by prep-HPLC [column: Phenomenex luna C18 150×25 mm×10 μm; mobile phase: [water (FA)-ACN]; gradient: 19%-49% B over 10 min] to afford the title compound (Example 36, 2.00 mg, 3.2% yield) as white solid; ¹H NMR (400 MHz, DMSO-d₆) δ=9.26 (d, J=7.2 Hz, 1H), 8.30 (br d, J=4.4 Hz, 1H), 8.18 (br d, J=8.0 Hz, 1H), 7.94 (d, J=8.0 Hz, 1H), 7.74 (t, J=7.6 Hz, 1H), 7.67-7.61 (m, 1H), 7.60-7.54 (m, 1H), 7.54-7.47 (m, 1H), 7.38-7.28 (m, 2H), 7.08-7.00 (m, 1H), 4.71-4.54 (m, 2H), 4.47 (br s, 1H), 4.09 (s, 2H), 3.74 (br dd, J=4.8, 13.6 Hz, 2H), 3.29-3.17 (m, 3H), 3.02-2.91 (m, 2H), 2.56 (br d, J=9.6 Hz, 2H), 2.30 (br d, J=13.2 Hz, 1H), 1.91 (br dd, J=6.0, 11.6 Hz, 3H), 1.82-1.75 (m, 3H), 1.63-1.56 (m, 2H); LCMS (ESI, M+1): m/z=688.3.

Step I. ethyl (3S,4S)-3-hydroxypiperidine-4-carboxylate: To a solution of 1-benzyl 4-ethyl (3S,4S)-3-hydroxypiperidine-1,4-dicarboxylate (700 mg, 1.0 equiv) in EtOAc (8.00 mL) was added Pd/C (1.00 g) under N₂ atmosphere. The reaction was degassed and purged with H₂ 3 times. The reaction was stirred at 25 °C for 4 hours under H₂ (50 psi). The mixture was filtered and concentrated to afford the title compound (300 mg, crude) as yellow solid; ¹H NMR (400 MHz, CHLOROFORM-d) δ=4.15 (q, J=7.2 Hz, 2H), 4.05 (br s, 1H), 3.06 (td, J=3.2, 12.8 Hz, 2H), 2.78 (br s, 2H), 2.67 (dd, J=1.6, 13.2 Hz, 1H), 2.56 (dt, J=2.8, 12.4 Hz, 1H), 2.51-2.41 (m, 1H), 1.92 (dq, J=4.4, 12.8 Hz, 1H), 1.74-1.64 (m, 1H), 1.24 (t, J=7.2 Hz, 3H).

Step J. ethyl (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate: To a mixture of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (250 mg, 1.0 equiv) and K₃PO₄ (300 mg, 3.0 equiv) in DMF (3.00 mL) were added ethyl (3S,4S)-3-hydroxypiperidine-4-carboxylate (245 mg, 3.0 equiv) and 4 Å molecular sieve (50.0 mg). The reaction was stirred at 60 °C for 2 hours. The mixture was filtered and purified by prep-HPLC [FA condition] to afford the title compound (220 mg, 77% yield) as yellow solid; LCMS (ESI, M+1): m/z=604.3.

Step K. (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylic acid: To a solution of ethyl (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate (90.0 mg, 1.0 equiv) in THF (1.50 mL) was added NaOH (2 M, 224 μL, 3.0 equiv). The reaction was stirred at 25 °C for 3 hours. The mixture was adjusted pH to 6. The mixture was washed with ethyl acetate (2×20.0 mL). The water layer was lyophilized to afford the title compound (60.0 mg, crude) as an off white solid; LCMS (ESI, M+1): m/z=576.3.

Step L. 3,4-difluorophenyl (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate: To a mixture of (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylic acid (50.0 mg, 1.0 equiv) and 3,4-difluorophenol (33.9 mg, 3.0 equiv) in DCM (1.00 mL) were added DMAP (15.9 mg, 1.5 equiv) and EDCI (25.0 mg, 1.5 equiv). The reaction was stirred at 20 °C for 4 hours. The mixture was diluted with water (20.0 mL) and extracted with DCM (2×20 mL). The combined organic layer were washed with brine (20.0 mL), dried over Na₂SO₄, concentrated and purified with prep-HPLC [column: Phenomenex luna C18 150×25 mm×10 μm; mobile phase: [water (FA)-ACN]; gradient: 19%-49% B over 10 min] to afford the title compound (Example 37, 2.00 mg, 3.2% yield) as white solid; ¹H NMR (400 MHz, DMSO-d6) δ=9.27 (d, J=7.2 Hz, 1H), 8.27 (br s, 1H), 8.19 (br d, J=8.4 Hz, 1H), 7.94 (d, J=8.0 Hz, 1H), 7.75 (t, J=8.0 Hz, 1H), 7.64 (dd, J=7.6, 12.0 Hz, 1H), 7.61-7.56 (m, 1H), 7.55-7.48 (m, 1H), 7.39-7.29 (m, 2H), 7.09-7.00 (m, 1H), 4.73-4.55 (m, 2H), 4.48 (br s, 1H), 4.11 (s, 2H), 3.74 (br dd, J=4.8, 13.2 Hz, 2H), 3.36-3.18 (m, 3H), 3.01-2.93 (m, 2H), 2.64-2.54 (m, 2H), 2.31 (br d, J=13.2 Hz, 1H), 1.97-1.87 (m, 3H), 1.81 (br dd, J=6.0, 11.2 Hz, 3H), 1.65-1.56 (m, 2H); LCMS (ESI, M+1): m/z=688.3.

### Example 38

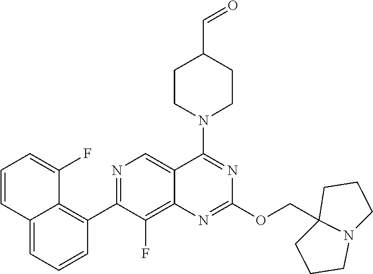 [SMILES: O=CC1CCN(c2nc(OCC34CCCN3CCC4)nc3c(F)c(-c4cccc5cccc(F)c45)ncc23)CC1]

### 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carbaldehyde

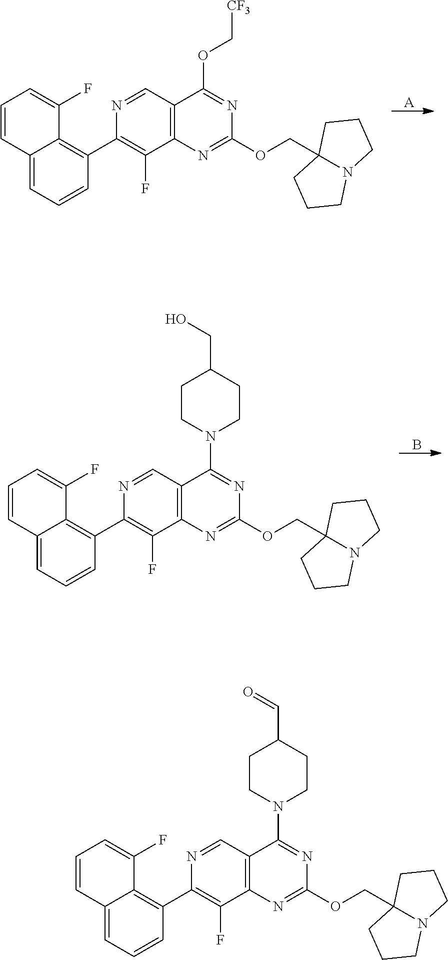

Step A. (1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)methanol: To a solution of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (100 mg, 1.0 equiv) and piperidin-4-ylmethanol (65.1 mg, 3.0 equiv) in DMF (1.00 mL) were added 4 Å molecular sieve (20.0 mg) and DIEA (73.1 mg, 3.0 equiv) The reaction was stirred at 50 °C for 12 hours. The mixture was purified with prep-HPLC [column: Phenomenex luna C18 150×25 mm×10 μm; mobile phase: [water (FA)-ACN]; gradient: 17%-47% B over 10 minutes] to afford the title compound (60.0 mg, 56% yield) as yellow solid; LCMS (ESI, M+1): m/z=546.3.

Step B. 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carbaldehyde: To a solution of (1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidin-4-yl)methanol (50.0 mg, 1.0 equiv) in DCM (1.00 mL) was added Dess-Martin reagent (77.7 mg, 2.0 equiv). The reaction was stirred at 20 °C for 2 hours. The mixture was quenched by Na₂SO₃ (20.0 mL at 0 °C) and extracted with ethyl acetate (3×20.0 mL). The combined organic layers were washed with brine (20.0 mL), dried over Na₂SO₄, concentrated and purified with prep-HPLC [column: Waters Xbridge 150×25 mm×5 μm; mobile phase: [water (ammonia hydroxide v/v)-ACN]; gradient: 31%-61% B over 9 min] to afford the title compound (5.60 mg, 11% yield) as white solid; ¹H NMR (400 MHz, DMSO-d₆) δ=9.67 (s, 1H), 9.08 (s, 1H), 8.18 (br d, J=7.6 Hz, 1H), 7.93 (d, J=8.0 Hz, 1H), 7.78-7.70 (m, 1H), 7.66-7.55 (m, 2H), 7.31 (br dd, J=7.2, 13.6 Hz, 1H), 4.64-4.50 (m, 1H), 4.40-4.34 (m, 1H), 4.05 (s, 2H), 3.62-3.54 (m, 1H), 2.96-2.89 (m, 2H), 2.85-2.73 (m, 1H), 2.56 (br s, 2H), 2.15-2.02 (m, 2H), 1.94-1.71 (m, 9H), 1.60-1.53 (m, 2H); LCMS (ESI, M+1): m/z=544.3.

### Example 39

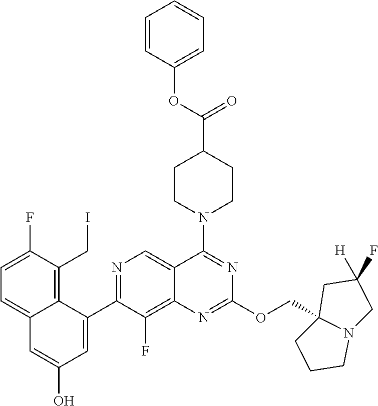 [SMILES: O=C(Oc1ccccc1)C1CCN(c2nc(OC[C@@]34CCCN3C[C@H](F)C4)nc3c(F)c(-c4cc(O)cc5ccc(F)c(CI)c45)ncc23)CC1]

### phenyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate

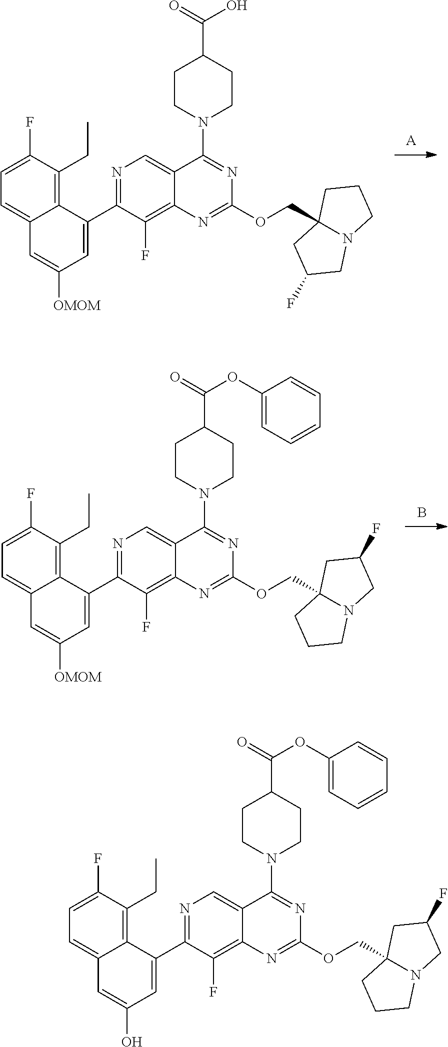

Step A. phenyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a mixture of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (230 mg, 1.0 equiv) and phenol (65.0 mg, 2.0 equiv) in DCM (2.0 mL) were added DMAP (63.3 mg, 1.5 equiv) and EDCI (99.3 mg, 1.5 equiv). The reaction was stirred at 25 °C for 2 hours. The mixture was concentrated and purified by reversed phase flash chromatography [C18, 0.1% formic acid condition] to afford the title compound (170 mg, 65.7% yield) as yellow solid; LCMS (ESI, M+1): m/z=742.4.

Step B. phenyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a solution of phenyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (150 mg, 1.0 equiv) in MeCN (1.0 mL) was added HCl·dioxane (4 M, 39 equiv). The reaction was stirred at 25 °C for 0.5 hours. The mixture was concentrated. The residue was diluted with water (2.0 mL). The mixture was adjusted to pH=7 with saturated NaHCO₃ aqueous. The mixture was extracted with ethyl acetate (3×5.0 mL). The combined organic layers were dried over sodium sulfate, concentrated and purified with prep-HPLC [Phenomenex luna C18 150×25 mm×10 μm; A: water (FA); B: ACN, B %: 25%-55% over 8 min] to afford the title compound (103 mg, 66% yield, 0.33 HCOOH) as white solid; ¹H NMR (400 MHz, dimethyl sulfoxide-d₆) δ=10.12-9.77 (m, 1H), 9.12 (s, 1H), 7.77 (dd, J=6.0, 9.2 Hz, 1H), 7.51-7.39 (m, 2H), 7.38-7.31 (m, 2H), 7.30-7.25 (m, 1H), 7.20-7.13 (m, 2H), 7.03 (d, J=2.4 Hz, 1H), 5.42-5.20 (m, 1H), 4.60-4.43 (m, 2H), 4.19 (dd, J=4.4, 10.4 Hz, 1H), 4.14-4.05 (m, 1H), 3.70-3.53 (m, 2H), 3.20-3.04 (m, 4H), 2.93-2.80 (m, 1H), 2.41-2.31 (m, 1H), 2.23 (br d, J=10.4 Hz, 2H), 2.19-2.07 (m, 3H), 2.06-1.92 (m, 3H), 1.91-1.75 (m, 3H), 0.73 (t, J=7.2 Hz, 3H); LCMS (ESI, M+1): m/z=698.3.

### Example 40

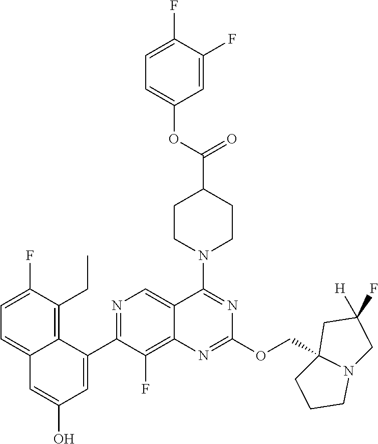 [SMILES: CCc1c(F)ccc2cc(O)cc(-c3ncc4c(N5CCC(C(=O)Oc6ccc(F)c(F)c6)CC5)nc(OC[C@@]56CCCN5C[C@H](F)C6)nc4c3F)c12]

### 3,4-difluorophenyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate

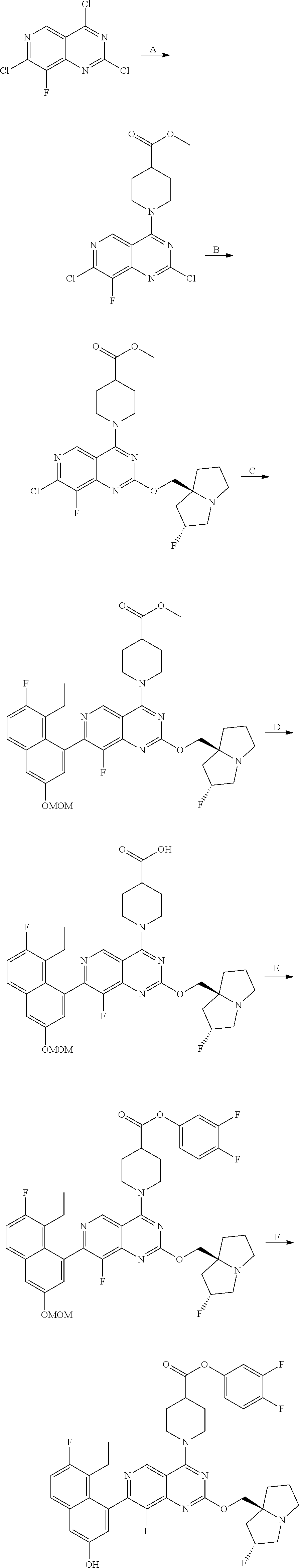

Step A. methyl 1-(2,7-dichloro-8-fluoropyrido[4,3-d]pyrimidin-4-yl)piperidine-4-carboxylate: To a solution of 2,4,7-trichloro-8-fluoropyrido[4,3-d]pyrimidine (900 mg, 1.0 equiv) and DIEA (921 mg, 2.0 equiv) in DCM (18.0 mL) was added methyl piperidine-4-carboxylate (408 mg, 0.80 equiv) at −40 °C. The reaction was stirred at −40 °C for 0.5 hours. The mixture was diluted with water (30.0 mL) and extracted with DCM (2×20 mL). The combined organic layers were dried over sodium sulfate, concentrated and purified by silica gel chromatography (petroleum ether/ethyl acetate=3/1 to 1/1) to afford the title compound (600 mg, 42% yield) as yellow solid; LCMS (ESI, M+1): m/z=359.1.

Step B. methyl 1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate; To a solution methyl 1-(2,7-dichloro-8-fluoropyrido[4,3-d]pyrimidin-4-yl)piperidine-4-carboxylate (500 mg, 1.0 equiv) in DMSO (0.5 mL) was added ((2R,7aS)-2-fluorotetrahydro-1H-pyrrolizin-7a(5H)-yl)methanol (665 mg, 3.0 equiv). The mixture was stirred at 100 °C for 3 hours. The mixture was filtered and purified by reversed phase flash chromatography [C18, 0.1% formic acid condition] to afford the title compound (600 mg, 88% yield) as yellow solid; LCMS (ESI, M+1): m/z=482.2.

Step C. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a mixture of methyl 1-(7-chloro-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (500 mg, 1.0 equiv) and 2-(8-ethyl-7-fluoro-3-(methoxymethoxy)naphthalen-1-yl)-4,4,5,5-tetramethyl-1,3,2-dioxaborolane (561 mg, 1.5 equiv) in methoxycyclopentane (9.0 mL) and water (3.0 mL) were added [2-(2-aminophenyl)phenyl]palladium bis(1-adamantyl)butylphosphane methanesulfonate (75.5 mg, 0.10 equiv) and Cs₂CO₃ (1.01 g, 3.0 equiv). The reaction was degassed and purged with nitrogen 3 times. The reaction was stirred at 90 °C for 5 hours. The mixture was filtered, concentrate and purified by reversed phase flash chromatography [C18, 0.1% formic acid condition] to afford the title compound (650 mg, 90% yield) as yellow solid; LCMS (ESI, M+1): m/z=680.5.

Step D. 1-(7-(8-ethyl-7-fluoro-3-(methoxymethoxy)naphthalen-1-yl)-8-fluoro-2-yl)piperidine-4-carboxylic acid: To a mixture of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (500 mg, 1.0 equiv) in water (1.5 mL) and THF (4.5 mL) was added NaOH (2 M in water, 4.0 equiv). The reaction was stirred at 25 °C for 2 hours. The mixture was adjusted to pH=7 with HCl (2 M) and extracted with ethyl acetate (3×10 mL). The combined organic layers were dried over sodium sulfate, filtered and concentrated to afford the title compound (480 mg, 96% yield) as yellow oil; LCMS (ESI, M+1): m/z=666.3.

Step E. 3,4-difluorophenyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a mixture of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (200 mg, 1.0 equiv) and 3,4-difluorophenol (78.2 mg, 2.0 equiv) in DCM (2 mL) were added DMAP (55.1 mg, 1.5 equiv) and EDCI (86.4 mg, 1.5 equiv). The reaction was stirred at 25 °C for 1 hour. The mixture was concentrated and purified by reversed phase flash chromatography [C18, 0.1% formic acid condition] to afford the title compound (200 mg, 81% yield) as yellow solid; LCMS (ESI, M+1): m/z=778.4

Step F. 3,4-difluorophenyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a mixture of 3,4-difluorophenyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (100 mg, 1.0 equiv) in MeCN (0.5 mL) was added HCl·dioxane (4 M, 31 equiv). The reaction was stirred at 25 °C for 0.5 hours. The mixture was concentrated. The residue was diluted with water (2.0 mL). The mixture was adjusted pH=7 with saturated NaHCO₃ aqueous solution and extracted with ethyl acetate (3× 5.0 mL). The combined organic layers were dried over sodium sulfate, concentrated and purified with prep-HPLC [Phenomenex luna C18 150×25 mm×10 μm; A: water (FA); B: ACN, B %: 28%-58% over 10 min] to afford the title compound (42.6 mg, 41% yield, 0.28 HCOOH) as white solid; ¹H NMR (400 MHz, dimethyl sulfoxide-d₆) δ=10.19-9.69 (m, 1H), 9.10 (s, 1H), 7.77 (dd, J=6.0, 9.2 Hz, 1H), 7.59-7.50 (m, 1H), 7.49-7.41 (m, 1H), 7.39-7.27 (m, 2H), 7.11-7.05 (m, 1H), 7.03 (d, J=2.4 Hz, 1H), 5.45-5.14 (m, 1H), 4.62-4.43 (m, 2H), 4.16 (dd, J=4.0, 10.4 Hz, 1H), 4.10-4.02 (m, 1H), 3.69-3.54 (m, 2H), 3.16-2.99 (m, 4H), 2.87-2.76 (m, 1H), 2.43-2.30 (m, 1H), 2.26-2.17 (m, 2H), 2.17-2.08 (m, 2H), 2.08-1.92 (m, 4H), 1.90-1.72 (m, 3H), 0.80-0.65 (m, 3H); LCMS (ESI, M+1): m/z=734.2.

### Example 41

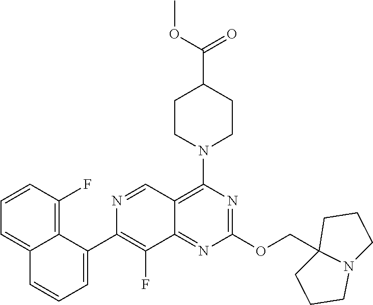 [SMILES: COC(=O)C1CCN(c2nc(OCC34CCCN3CCC4)nc3c(F)c(-c4cccc5cccc(F)c45)ncc23)CC1]

### methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

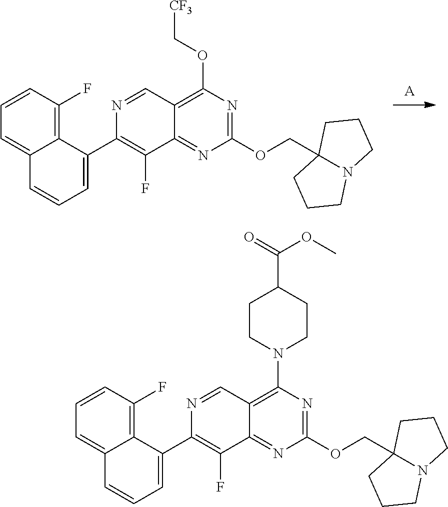

Step A: methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (50.1 mg, 1.0 equiv) and methyl piperidine-4-carboxylate (27.0 mg, 2.0 equiv) in DMF (1 mL) were added N-ethyl-N,N-diisopropylamine (73.2 mg, 6.0 equiv) and 4 Å molecular sieve (10.0 mg). The reaction was stirred at 50 °C for 16 hours. The mixture was diluted with water (15 mL) and extracted with ethyl acetate (3×15 mL). The combined organic layers were washed with brine (2×15 mL), dried over anhydrous sodium sulfate, concentrated, and purified with prep-HPLC [Phenomenex luna C18 150×25 mm×10 μm; A: water (FA), B: ACN, B %: 22%-52% over 7 mins] to afford the title compound (11.4 mg, 20% yield, 0.6 HCCOH) as white solid; ¹H NMR (400 MHz, METHANOL-d₄) δ=9.08 (s, 1H), 8.12 (br d, J=8.4 Hz, 1H), 7.86 (d, J=8.4 Hz, 1H), 7.76-7.66 (m, 1H), 7.65-7.58 (m, 1H), 7.54 (dt, J=5.2, 8.0 Hz, 1H), 7.19 (dd, J=7.6, 13.2 Hz, 1H), 4.62 (br d, J=13.6 Hz, 2H), 4.52 (s, 2H), 3.79-3.70 (m, 3H), 3.69-3.60 (m, 2H), 3.50-3.42 (m, 2H), 3.12-3.02 (m, 2H), 2.92-2.83 (m, 1H), 2.28-2.02 (m, 8H), 2.02-1.90 (m, 4H); LCMS (ESI, M+1): m/z=574.5.

### Example 42

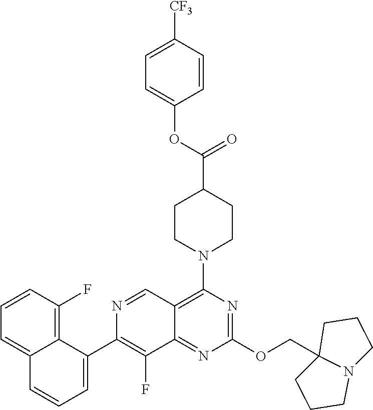 [SMILES: O=C(Oc1ccc(C(F)(F)F)cc1)C1CCN(c2nc(OCC34CCCN3CCC4)nc3c(F)c(-c4cccc5cccc(F)c45)ncc23)CC1]

### 4-(trifluoromethyl)phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

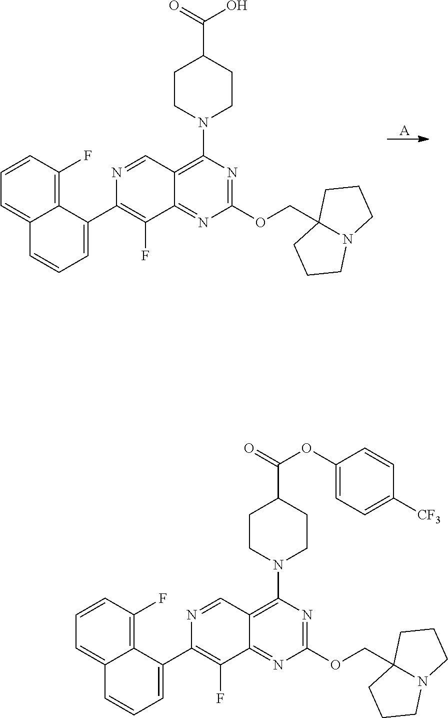

Step A. 4-(trifluoromethyl)phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (45.0 mg, 1.0 equiv) and 4-(trifluoromethyl) phenol (39.1 mg, 3.0 equiv) in DCM (5 mL) were added 4-dimethylaminopyridine (19.7 mg, 2.0 equiv) and 1-ethyl-3-(3-dimethylaminopropyl)carbodiimide (30.8 mg, 2.0 equiv). The reaction was stirred at 40 °C for 16 hours. The mixture was concentrated and purified with prep-HPLC [Phenomenex luna C18 150×25 mm×10 μm, A: water (FA), B: ACN, B %: 35%-65% over 7 mins] to afford the title compound (23.5 mg, 39% yield, 0.1 HCOOH) as white solid; ¹H NMR (400 MHz, DMSO-d₆) δ=9.18 (s, 1H), 8.20 (br d, J=8.4 Hz, 1H), 7.95 (d, J=8.0 Hz, 1H), 7.84 (d, J=8.8 Hz, 2H), 7.79-7.72 (m, 1H), 7.67-7.56 (m, 2H), 7.43 (d, J=8.4 Hz, 2H), 7.32 (dd, J=7.2, 13.2 Hz, 1H), 4.64-4.46 (m, 4H), 3.64 (br t, J=12.4 Hz, 2H), 3.51-3.40 (m, 2H), 3.24-3.10 (m, 3H), 2.26 (br d, J=10.4 Hz, 2H), 2.15 (br dd, J=6.0, 12.0 Hz, 2H), 2.09-1.91 (m, 8H); LCMS (ESI, M+1): m/z=704.4.

### Example 43

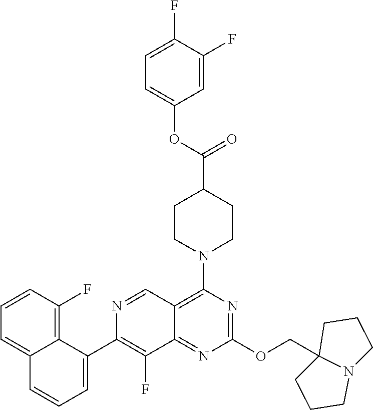 [SMILES: O=C(Oc1ccc(F)c(F)c1)C1CCN(c2nc(OCC34CCCN3CCC4)nc3c(F)c(-c4cccc5cccc(F)c45)ncc23)CC1]

### 3,4-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

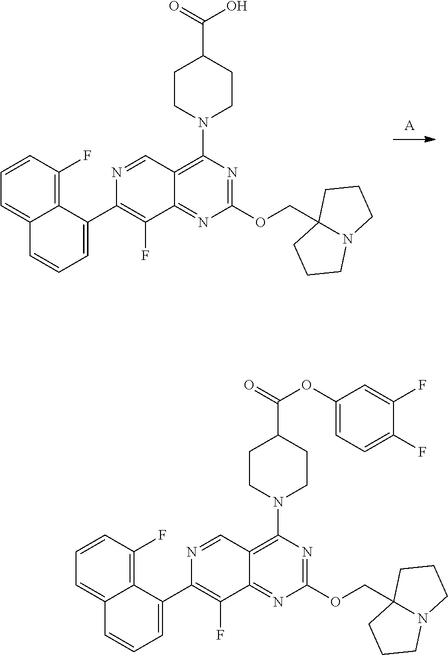

Step A. 3,4-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (50.0 mg, 1.0 equiv) and 3,4-difluorophenol (34.9 mg, 3.0 equiv) in DCM (3 mL) were added 4-dimethylaminopyridine (21.8 mg, 2.0 equiv) and 1-ethyl-3-(3-dimethylaminopropyl)carbodiimide (34.3 mg, 2.0 equiv). The reaction was stirred at 35 °C for 16 hours. The mixture was concentrated and purified by prep-HPLC [Phenomenex luna C18 150×25 mm×10 μm; A: water (FA), B: ACN, B %: 32%-62% over 7 mins] to afford the title compound (21.5 mg, 33% yield) as yellow solid; ¹H NMR (400 MHz, DMSO-d6) δ=9.17 (s, 1H), 8.20 (br d, J=8.4 Hz, 1H), 7.94 (d, J=8.4 Hz, 1H), 7.82-7.72 (m, 1H), 7.66-7.62 (m, 1H), 7.61-7.56 (m, 1H), 7.55-7.48 (m, 1H), 7.48-7.41 (m, 1H), 7.32 (dd, J=7.6, 13.2 Hz, 1H), 7.12-7.04 (m, 1H), 4.62-4.41 (m, 4H), 3.69-3.57 (m, 2H), 3.38 (br s, 2H), 3.18-3.04 (m, 3H), 2.23 (br d, J=10.4 Hz, 2H), 2.13 (br dd, J=6.4, 11.6 Hz, 2H), 2.08-1.88 (m, 8H); LCMS (ESI, M+1): m/z=672.2.

### Example 44

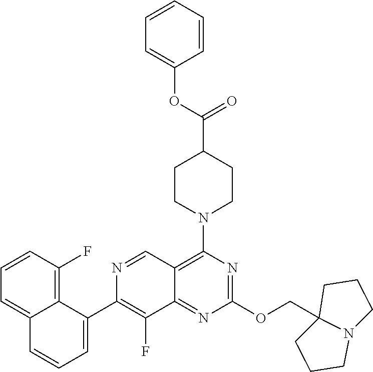 [SMILES: O=C(Oc1ccccc1)C1CCN(c2nc(OCC34CCCN3CCC4)nc3c(F)c(-c4cccc5cccc(F)c45)ncc23)CC1]

### phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate

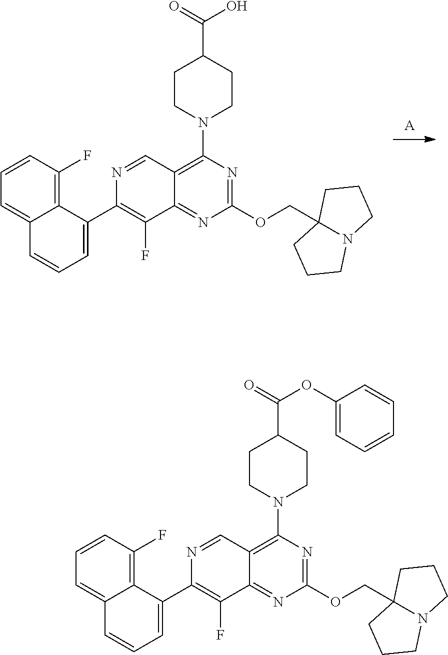

Step A. phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-4-carboxylic acid (50.0 mg, 1.0 equiv) and phenol (25.2 mg, 3.0 equiv) in DCM (5 mL) were added 4-dimethylaminopyridine (21.8 mg, 2.0 equiv) and 1-ethyl-3-(3-dimethylaminopropyl)carbodiimide (34.3 mg, 2.0 equiv). The reaction was stirred at 40 °C for 16 hours. The mixture was concentrated and purified with prep-HPLC [Phenomenex luna C18 150×25 mm×10 μm; A: water (FA), B: ACN, B %: 30%-50% over 10 mins] to afford the title compound (2.07 mg, 3.6% yield) as white solid; ¹H NMR (400 MHz, DMSO-d6) δ=9.19 (s, 1H), 8.21-8.17 (m, 1H), 7.98-7.89 (m, 1H), 7.81-7.70 (m, 1H), 7.68-7.56 (m, 2H), 7.50-7.39 (m, 2H), 7.36-7.25 (m, 2H), 7.17 (d, J=8.0 Hz, 2H), 4.60-4.44 (m, 2H), 4.28-4.15 (m, 2H), 3.66-3.57 (m, 2H), 3.15-3.04 (m, 3H), 2.80-2.68 (m, 2H), 2.28-2.20 (m, 2H), 2.03-1.79 (m, 8H), 1.73-1.63 (m, 2H); LCMS (ESI, M+1): m/z=636.5.

### Example 45

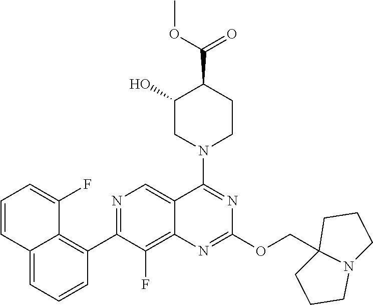 [SMILES: COC(=O)[C@H]1CCN(c2nc(OCC34CCCN3CCC4)nc3c(F)c(-c4cccc5cccc(F)c45)ncc23)C[C@@H]1O]

### methyl (3R,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate

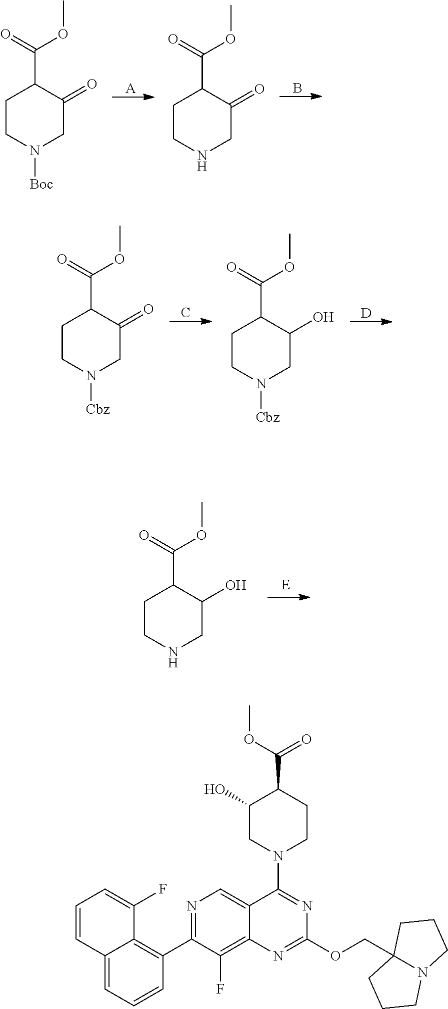

Step A. methyl 3-oxopiperidine-4-carboxylate: To a solution of 1-(tert-butyl) 4-methyl 3-oxopiperidine-1,4-dicarboxylate (2.00 g, 1.0 equiv) in ACN (20 mL) was added HCl·dioxane (4 M, 1.94 mL, 1.0 equiv). The reaction was stirred at 0 °C for 1 hour. The mixture was concentrated to afford the title compound (1.20 g, crude) as white oil.

Step B. 1-benzyl 4-methyl 3-oxopiperidine-1,4-dicarboxylate: To a solution of methyl 3-oxopiperidine-4-carboxylate (1.20 g, 1.0 equiv) and benzyl carbonochloridate (1.56 g, 1.2 equiv) in THF (12 mL) was added TEA (1.08 g, 1.4 equiv) at 0 °C. The reaction was stirred at 25 °C for 2 hours. The mixture was quenched with water (15 mL) and extracted with ethyl acetate (3×15 mL). The combined organic layers were dried over sodium sulfate, concentrated and purified with prep-HPLC (column: Phenomenex luna C18 250×50 mm×10 μm; A: water (FA), B: ACN, B %: 38%-68% B over 21 min) to afford the title compound (1.27 g, 57% yield) as red oil; LCMS (ESI, M+1): m/z=292.2.

Step C. 1-benzyl 4-methyl 3-hydroxypiperidine-1,4-dicarboxylate: To a solution of 1-benzyl 4-methyl 3-oxopiperidine-1,4-dicarboxylate (1.00 g, 1.0 equiv) in EtOH (10 mL) was added NaBH₄ (64.9 mg, 0.5 equiv) at 0 °C. The reaction was stirred at 0 °C for 5 minutes. The mixture was quenched by saturated NH₄Cl solution (20 mL) and extracted with ethyl acetate (3×20 mL). The combined organic layers were dried over sodium sulfate, concentrated and purified by reversed phase flash [C18, 0.1% formic acid condition] to afford the title compound (500 mg, 44% yield) as colorless oil; LCMS (ESI, M+1): m/z=293.9.

Step D. methyl 3-hydroxypiperidine-4-carboxylate: To a solution of 1-benzyl 4-methyl 3-hydroxypiperidine-1,4-dicarboxylate (500 mg, 1.0 equiv) in isopropanol (5 mL) was added Pd/C (100 mg, 10% purity). The reaction was degassed and purged with H₂ 3 times. The reaction was stirred at 25 °C for 1 hour under H₂ (15 psi). The mixture was filtered and concentrated to afford the title compound (270 mg, crude) as yellow oil.

Step E. methyl (3R,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate: To a solution of methyl 3-hydroxypiperidine-4-carboxylate (135 mg, 3.3 equiv) in DMF (1 mL) was added DIEA (164 mg, 5.0 equiv) and 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (135 mg, 1.0 equiv). The reaction was stirred at 40 °C for 12 hours. The mixture was filtered and purified by prep-HPLC [column: Phenomenex luna C18 150×40 mm×15 μm; A: water (FA), B: ACN, B %: 12%-42% B over 11 min] and SFC separation [column: ChiralPak IH, 250×50 mm, 10 μm; A: CO₂, B: MeOH (0.1% NH₃H₂O), B %: 40%-40% B over 5.1 min] to afford the title compound (1.39 mg, 0.86% yield) as yellow solid; ¹H NMR (400 MHz, METHANOL-d4) δ=9.12 (s, 1H), 8.12 (br d, J=8.4 Hz, 1H), 7.86 (d, J=8.0 Hz, 1H), 7.75-7.66 (m, 1H), 7.63-7.49 (m, 2H), 7.19 (dd, J=7.6, 12.8 Hz, 1H), 4.63 (br d, J=4.4 Hz, 1H), 4.55-4.49 (m, 1H), 4.41 (s, 2H), 4.15-4.06 (m, 1H), 3.75 (s, 3H), 3.69-3.58 (m, 1H), 3.50-3.34 (m, 2H), 2.96-2.84 (m, 2H), 2.74 (ddd, J=4.4, 8.8, 10.8 Hz, 1H), 2.25-2.13 (m, 3H), 2.08-1.80 (m, 8H); LCMS (ESI, M+1): m/z=590.5.

### Example 46

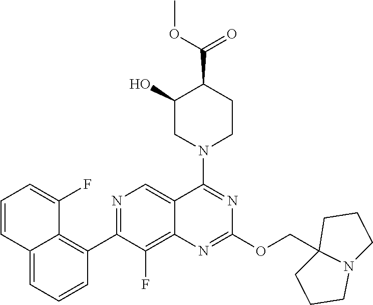 [SMILES: COC(=O)[C@H]1CCN(c2nc(OCC34CCCN3CCC4)nc3c(F)c(-c4cccc5cccc(F)c45)ncc23)C[C@H]1O]

### methyl (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate

### Example 47

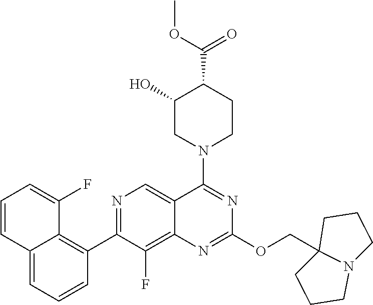 [SMILES: COC(=O)[C@@H]1CCN(c2nc(OCC34CCCN3CCC4)nc3c(F)c(-c4cccc5cccc(F)c45)ncc23)C[C@@H]1O]

### methyl (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate

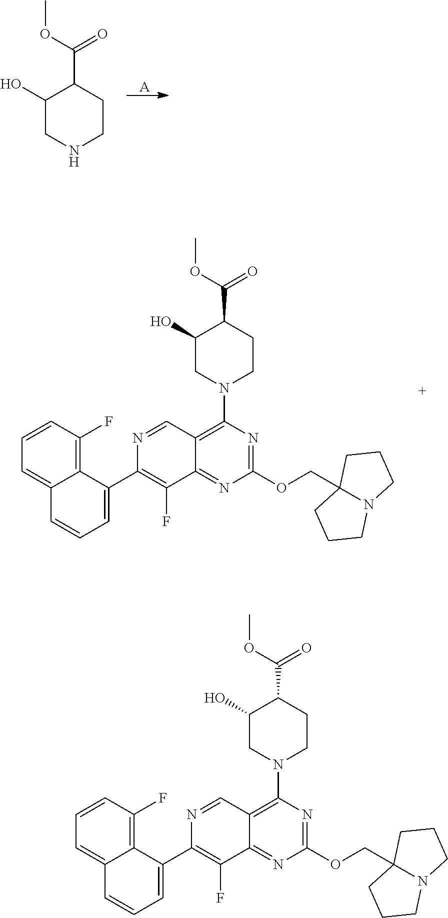

Step A. methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate and methyl (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-cis-hydroxypiperidine-4-carboxylate: To a solution of methyl 3-hydroxypiperidine-4-carboxylate (135 mg, 3.3 equiv) in DMF (1 mL) were added DIEA (164 mg, 5.0 equiv) and 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (135 mg, 1.0 equiv). The reaction was stirred at 40 °C for 12 hours. The mixture was filtered and purified by prep-HPLC [column: Phenomenex luna C18 150×25 mm×10 μm; A: water (FA), B: ACN, B %: 18%-48% B over 9 min] and SFC separation [column: DAICEL CHIRALCEL OX, 250×30 mm, 10 μm; A: CO₂, B: MeOH (0.1% NH₃H₂O), B %: 50%-50% B over 3.7 min] to afford two isomers.

methyl (3S,4S)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate (Example 46, 3.42 mg, 2.0% yield, HCOOH salt) as off-white solid; SFC: 94.4% ee. Column:Lux 3 μm Cellulose-4 50×4.6 mm I.D., 3 μm, mobile phase 40% (MeOH:ACN=4:1) (0.05% DEA), flow rate: 3 mL/min, detector: 220 nm, t_{R}: 2.211 min; ¹H NMR (400 MHz, METHANOL-d4) δ=9.24 (d, J=11.2 Hz, 1H), 8.11 (br d, J=8.0 Hz, 1H), 7.92-7.79 (m, 1H), 7.73-7.66 (m, 1H), 7.64-7.48 (m, 2H), 7.26-7.13 (m, 1H), 4.82 (br d, J=2.4 Hz, 1H), 4.74 (br d, J=13.6 Hz, 1H), 4.45 (br s, 1H), 4.41-4.35 (m, 2H), 3.76-3.66 (m, 4H), 3.28-3.21 (m, 2H), 2.98-2.80 (m, 3H), 2.44-2.28 (m, 1H), 2.20-2.10 (m, 2H), 2.07-1.76 (m, 8H); LCMS (ESI, M+1): m/z=590.6;

methyl (3R,4R)-1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-3-hydroxypiperidine-4-carboxylate (Example 47, 6.40 mg, 4.1% yield, HCOOH salt) as white solid; SFC: 94.4% ee. Column:Lux 3 μm Cellulose-4 50×4.6 mm I.D., 3 μm, mobile phase 40% (MeOH:ACN=4:1) (0.05% DEA), flow rate: 3 mL/min, detector: 220 nm, t_{R}: 2.719 min; ¹H NMR (400 MHz, METHANOL-d4) δ=9.25 (d, J=11.2 Hz, 1H), 8.11 (br d, J=8.4 Hz, 1H), 7.85 (d, J=8.0 Hz, 1H), 7.73-7.67 (m, 1H), 7.63-7.49 (m, 2H), 7.19 (dd, J=7.6, 13.2 Hz, 1H), 4.83-4.78 (m, 1H), 4.74 (br d, J=13.6 Hz, 1H), 4.49-4.36 (m, 3H), 3.77-3.65 (m, 4H), 3.29-3.25 (m, 1H), 2.98-2.85 (m, 3H), 2.44-2.28 (m, 1H), 2.23-2.12 (m, 2H), 2.11-1.93 (m, 6H), 1.92-1.82 (m, 3H); LCMS (ESI, M+1): m/z=590.5.

### Example 48

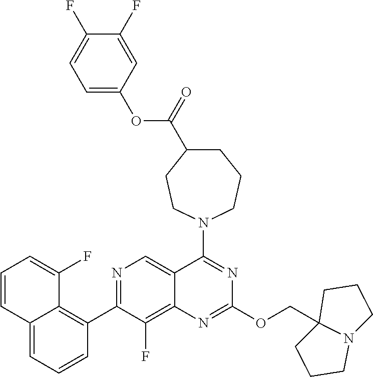 [SMILES: O=C(Oc1ccc(F)c(F)c1)C1CCCN(c2nc(OCC34CCCN3CCC4)nc3c(F)c(-c4cccc5cccc(F)c45)ncc23)CC1]

### 3,4-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylate

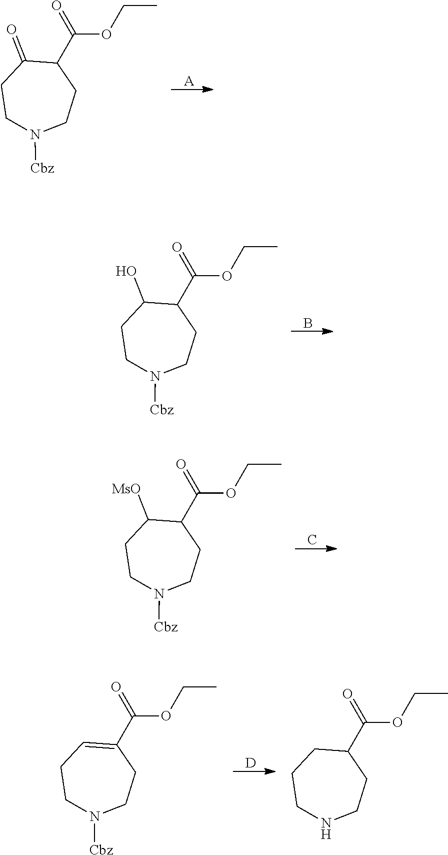

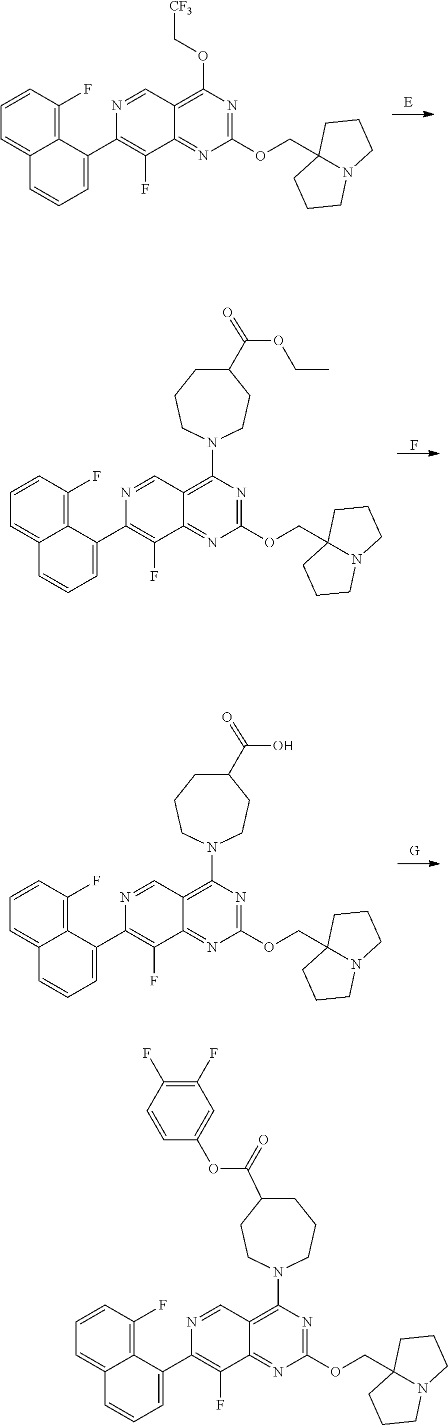

Step A: 1-benzyl 4-ethyl 5-hydroxyazepane-1,4-dicarboxylate. To a solution of 1-benzyl 4-ethyl 5-oxoazepane-1,4-dicarboxylate (11.0 g, 1.0 equiv) in THF (110 mL) was added NaBH₄ (1.39 g, 1.1 equiv) at 0 °C. The reaction was stirred at 0 °C for 1 hour. The mixture was quenched with saturated NH₄Cl solution (100 mL) at 0 °C and extracted with ethyl acetate (3×80 mL). The combined organic layers were washed with brine (100 mL), dried over anhydrous sodium sulfate, concentrated, and purified by column chromatography [SiO₂, Petroleum ether/Ethyl acetate=3/1 to 1/1] to afford the title compound (5.00 g, 44% yield) as colorless oil; ¹H NMR (400 MHz, DMSO-d₆) δ=7.37-7.27 (m, 5H), 5.06 (s, 2H), 4.81-4.72 (m, 1H), 4.11-3.95 (m, 2H), 3.57-3.37 (m, 3H), 3.33-3.21 (m, 1H), 2.47-2.40 (m, 1H), 2.11-1.99 (m, 1H), 1.95-1.77 (m, 2H), 1.75-1.61 (m, 1H), 1.17 (dt, J=2.4, 7.2 Hz, 3H).

Step B: 1-benzyl 4-ethyl 5-((methylsulfonyl)oxy) azepane-1,4-dicarboxylate. To a solution of 1-benzyl 4-ethyl 5-hydroxyazepane-1,4-dicarboxylate (7.00 g, 1.0 equiv) in DCM (70 mL) were added TEA (6.61 g, 9.1 mL, 3.0 equiv) and MsCl (6.47 g, 2.6 equiv). The reaction was stirred at 25 °C for 1 hour. The mixture was quenched with ice water (50 mL) slowly at 0 °C. The mixture was diluted with water (20 mL) and extracted with DCM (3×50 mL). The combined organic layers were washed with brine (50 mL), dried over anhydrous sodium sulfate, concentrated to afford the title compound (10.0 g, crude) as yellow oil; ¹H NMR (400 MHz, DMSO-d₆) δ=7.38-7.25 (m, 5H), 5.33-5.23 (m, 1H), 5.07 (s, 2H), 4.16-3.96 (m, 2H), 3.57-3.35 (m, 4H), 3.13 (d, J=3.6 Hz, 2H), 3.10-3.05 (m, 1H), 2.92 (br d, J=10.0 Hz, 1H), 2.32-2.22 (m, 1H), 2.10-1.90 (m, 3H), 1.20-1.14 (m, 3H).

Step C: 1-benzyl 4-ethyl 2,3,6,7-tetrahydro-1H-azepine-1,4-dicarboxylate. To a solution of 1-benzyl 4-ethyl 5-((methylsulfonyl)oxy) azepane-1,4-dicarboxylate (9.00 g, 1.0 equiv) in THF (90 mL) was added DBU (6.86 g, 2.0 equiv). The reaction was stirred at 80 °C for 1 hour. The mixture was diluted with water (150 mL) and extracted with ethyl acetate (3×50 mL). The combined organic layers were washed with brine (100 mL), dried over anhydrous sodium sulfate, concentrated, and purified by column chromatography [SiO₂, Petroleum ether/Ethyl acetate=10/1 to 3/1] to afford the title compound (4.00 g, 54% yield) as yellow oil; ¹H NMR (400 MHz, DMSO-d₆) δ=7.40-7.27 (m, 5H), 7.02 (br d, J=1.6 Hz, 1H), 5.10 (s, 2H), 4.10 (q, J=7.2 Hz, 2H), 3.51 (br s, 4H), 2.64-2.58 (m, 2H), 2.44 (br d, J=5.2 Hz, 2H), 1.21 (t, J=7.2 Hz, 3H).

Step D: ethyl azepane-4-carboxylate. To a solution of 1-benzyl 4-ethyl 2,3,6,7-tetrahydro-1H-azepine-1,4-dicarboxylate (3.00 g, 1.0 equiv) in THF (50 mL) was added Pd/C (700 mg, 10% purity). The reaction was degassed and purged with H₂ 3 times. The reaction was stirred at 40 °C for 6 hours under H₂ (50 Psi). The mixture was filtered and concentrated to afford the title compound (1.50 g, 88% yield) as yellow oil; ¹H NMR (400 MHz, DMSO-d₆) δ=4.03 (q, J=7.2 Hz, 2H), 2.83-2.71 (m, 2H), 2.71-2.58 (m, 2H), 2.57-2.52 (m, 1H), 1.91-1.79 (m, 2H), 1.72-1.57 (m, 3H), 1.54-1.39 (m, 1H), 1.16 (t, J=7.2 Hz, 3H).

Step E: 8-fluoro-7-(8-fluoronaphthalen-1-yl)-2-((tetrahydro-1H-pyrrolizin-7a(5H)-yl)methoxy)-4-(2.2,2-trifluoroethoxy)pyrido[4,3-d]pyrimidine. To a mixture of 8-fluoro-7-(8-fluoro-1-naphthyl)-2-(1,2,3,5,6,7-hexahydropyrrolizin-8-ylmethoxy)-4-(2,2,2-trifluoroethoxy)pyrido[4,3-d]pyrimidine (1.00 g, 1.0 equiv) and 4 Å molecular sieve (1.00 g) in DMF (20 mL) were added K₃PO₄ (731 mg, 1.8 equiv) and ethyl azepane-4-carboxylate (1.61 g, 5.0 equiv). The reaction was stirred at 50 °C for 1 hour. The mixture was filtered, concentrated and purified by reversed phase flash [water (0.1% FA)/ACN] to afford the title compound (730 mg, 61% yield) as yellow solid; ¹H NMR (400 MHz, DMSO-d₆) δ=9.15 (s, 1H), 8.17 (br d, J=8.4 Hz, 1H), 7.97-7.91 (m, 1H), 7.77-7.70 (m, 1H), 7.66-7.53 (m, 2H), 7.35-7.26 (m, 1H), 4.19-4.08 (m, 2H), 4.06-4.03 (m, 3H), 3.95-3.83 (m, 2H), 2.99-2.87 (m, 2H), 2.68-2.60 (m, 1H), 2.57-2.52 (m, 2H), 2.29-2.17 (m, 1H), 2.07 (br d, J=11.6 Hz, 2H), 1.98-1.84 (m, 5H), 1.83-1.65 (m, 5H), 1.56 (td, J=7.2, 12.0 Hz, 2H), 1.16-1.12 (m, 3H).

Step F: 1-(8-fluoro-7-(8-fluoronaphthalen-1-yl)-2-((tetrahydro-1H-pyrrolizin-7a(5H)-yl) methoxy)pyrido[4,3-d]pyrimidin-4-yl) azepane-4-carboxylic acid. To a solution of ethyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylate (390 mg, 1.0 equiv) in THF (4.0 mL) and MeOH (4.0 mL) was added NaOH solution (648 μL, 2.0 equiv, 2 M). The reaction was stirred at 25 °C for 1 hour. The mixture was quenched with HCl (3 M) and concentrated to afford the title compound (500 mg, HCl) as white solid; ¹H NMR (400 MHz, DMSO-d₆) δ=9.15 (s, 1H), 8.53-8.50 (m, 1H), 8.54 (s, 1H), 8.17-7.76 (m, 1H), 7.66-7.55 (m, 2H), 7.30 (dd, J=7.6, 12.8 Hz, 1H), 4.21-4.08 (m, 2H), 3.92-3.82 (m, 2H), 2.95-2.87 (m, 2H), 2.56-2.53 (m, 2H), 2.47-2.36 (m, 1H), 1.94 (br s, 5H), 1.88 (br dd, J=5.6, 12.0 Hz, 3H), 1.83-1.69 (m, 5H), 1.60-1.52 (m, 3H).

Step G: 3,4-difluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylate: To a solution of 1-(8-fluoro-7-(8-fluoronaphthalen-1-yl)-2-((tetrahydro-1H-pyrrolizin-7a(5H)-yl) methoxy)pyrido[4,3-d]pyrimidin-4-yl) azepane-4-carboxylic acid (95.0 mg, 1.0 equiv) and 3,4-difluorophenol (64.6 mg, 3.0 equiv) in DCM (1.0 mL) were added DMAP (40.5 mg, 2.0 equiv) and EDCI (63.5 mg, 2.0 equiv). The reaction was stirred at 50 °C for 1 hour. The mixture was concentrated and purified by prep-HPLC [column: YMC-Actus Triart C18 150×30 mm×7 μm; mobile phase: [water (FA)-ACN]; gradient: 30%-60% B over 10 mins] and prep-HPLC [column: YMC-Actus Triart C18 150×30 mm×7 μm; mobile phase: [water (FA)-ACN]; gradient: 30%-60% B over 10 mins] to afford the title compound (19.5 mg, 17% yield, 0.2 HCOOH) as off-white solid; ¹H NMR (400 MHz, DMSO-d₆) δ=9.21-9.16 (m, 1H), 8.18 (br d, J=8.4 Hz, 1H), 7.93 (d, J=8.4 Hz, 1H), 7.77-7.70 (m, 1H), 7.67-7.55 (m, 2H), 7.49 (br d, J=9.2 Hz, 1H), 7.42-7.26 (m, 2H), 7.05-6.98 (m, 1H), 4.31-4.19 (m, 2H), 4.13-4.05 (m, 2H), 4.03-3.84 (m, 2H), 3.75-3.62 (m, 1H), 3.61-3.46 (m, 2H), 2.94 (br dd, J=3.2, 5.6 Hz, 2H), 2.16-2.02 (m, 4H), 1.93-1.86 (m, 3H), 1.78-1.65 (m, 4H), 1.63-1.54 (m, 3H); LCMS (ESI, M+1): m/z=686.4.

### Example 49

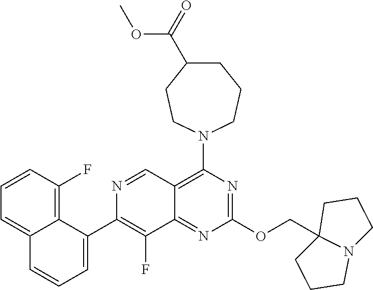 [SMILES: COC(=O)C1CCCN(c2nc(OCC34CCCN3CCC4)nc3c(F)c(-c4cccc5cccc(F)c45)ncc23)CC1]

### methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylate

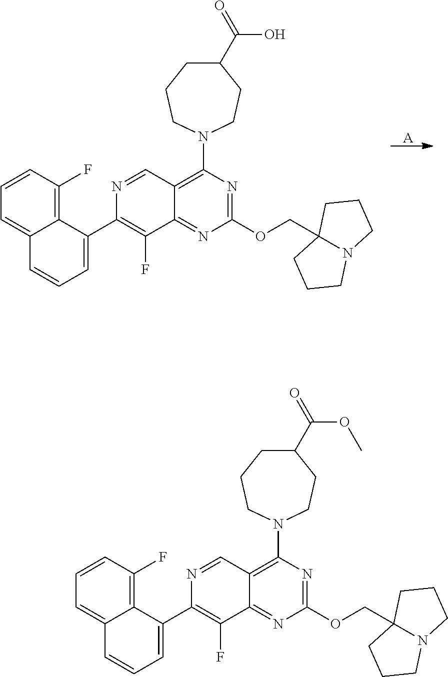

Step A. methyl 1-(8-fluoro-7-(8-fluoronaphthalen-1-yl)-2-((tetrahydro-1H-pyrrolizin-7a (5H)-yl)methoxy)pyrido[4,3-d]pyrimidin-4-yl) azepane-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylic acid (50.0 mg, 1.0 equiv) in dichloromethane (1 mL) were added oxalyl chloride (22.1 mg, 2.0 equiv) and dimethylformamide (6.37 mg, 1.0 equiv) at 0 °C. The reaction was stirred at 0 °C for 0.5 hour. The mixture was concentrated. To the residue was added methanol (27.9 mg, 10.0 equiv) dropwise at 0 °C. The reaction was stirred at 25 °C for 4 hours. The mixture was filtered and the filtrate was purified by prep-HPLC [YMC-Actus Triart C18 150*30 mm*7 μm; mobile phase: [water (FA)-ACN]; B %: 23%-53% over 4 mins] to afford the title compound (5.79 mg, 11% yield, 0.3 HCOOH) as white solid; ¹H NMR (400 MHz, DMSO+D₂O) δ=9.12 (s, 1H), 8.15 (br d, J=8.4 Hz, 1H), 7.90 (d, J=8.4 Hz, 1H), 7.72 (br t, J=8.0 Hz, 1H), 7.64-7.52 (m, 2H), 7.32-7.24 (m, 1H), 4.22-4.14 (m, 3H), 4.11-4.07 (m, 1H), 3.54 (br d, J=6.0 Hz, 3H), 3.02 (br d, J=4.4 Hz, 2H), 2.67-2.63 (m, 4H), 2.22-2.18 (m, 1H), 2.08-2.04 (m, 2H), 2.01-1.87 (m, 4H), 1.86-1.81 (m, 2H), 1.80-1.72 (m, 3H), 1.67-1.59 (m, 3H); LCMS (ESI, M+1): m/z=588.5

### Example 50

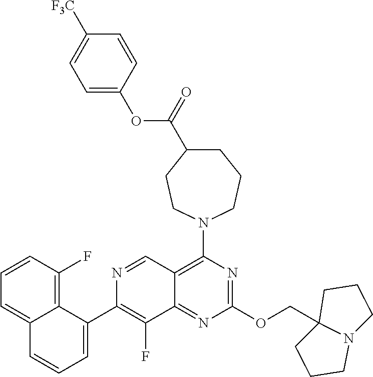 [SMILES: O=C(Oc1ccc(C(F)(F)F)cc1)C1CCCN(c2nc(OCC34CCCN3CCC4)nc3c(F)c(-c4cccc5cccc(F)c45)ncc23)CC1]

### 4-(trifluoromethyl)phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylate

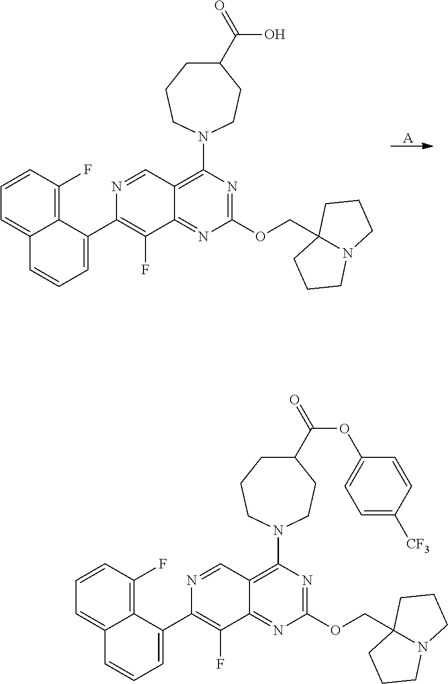

Step A. 4-(trifluoromethyl)phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl) azepane-4-carboxylic acid (60.0 mg, 1.0 equiv) in dichloromethane (1.0 mL) were added 4-(trifluoromethyl) phenol (50.9 mg, 3.0 equiv), 4-dimethylaminopyridine (25.6 mg, 2.0 equiv) and 1-ethyl-3-(3-dimethylaminopropyl)carbodiimide (40.1 mg, 2.0 equiv). The reaction was stirred at 25 °C for 4 hours. The mixture was concentrated and purified by prep-HPLC [YMC-Actus Triart C18 150*30 mm*7 μm; mobile phase: [water (FA)-ACN]; B %: 33%-63% over 4 mins] to afford the title compound (34.9 mg, 46% yield, 0.6 HCOOH) as white solid; ¹H NMR (400 MHz, DMSO+D₂O) δ=9.18 (s, 1H), 8.16 (br d, J=8.0 Hz, 1H), 7.91 (d, J=8.4 Hz, 1H), 7.79-7.71 (m, 3H), 7.62-7.54 (m, 2H), 7.32-7.24 (m, 3H), 4.33-4.25 (m, 3H), 4.14 (br s, 1H), 4.07-3.91 (m, 3H), 3.16 (br d, J=4.8 Hz, 1H), 3.00-2.96 (m, 1H), 2.83-2.79 (m, 2H), 2.28-2.12 (m, 4H), 2.03-1.95 (m, 3H), 1.91 (br dd, J=6.0, 12.0 Hz, 2H), 1.86-1.78 (m, 3H), 1.78-1.72 (m, 2H); LCMS (ESI, M+1): m/z=718.3.

### Example 51

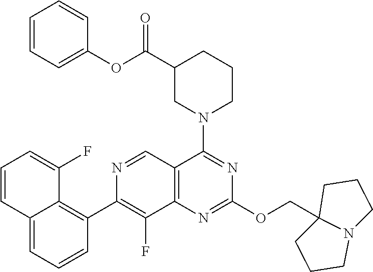 [SMILES: O=C(Oc1ccccc1)C1CCCN(c2nc(OCC34CCCN3CCC4)nc3c(F)c(-c4cccc5cccc(F)c45)ncc23)C1]

### phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylate

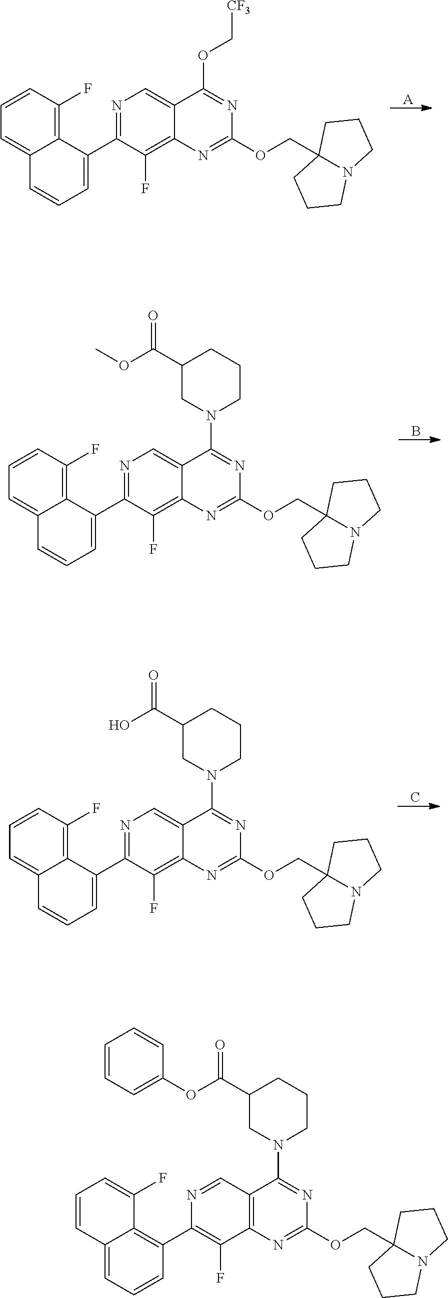

Step A. methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylate: To a solution of 8-fluoro-7-(8-fluoronaphthalen-1-yl)-2-((tetrahydro-1H-pyrrolizin-7a(5H)-yl)methoxy)-4-(2,2,2-trifluoroethoxy)pyrido[4,3-d]pyrimidine (1 g, 1.0 equiv) in DMAC (10 mL) were added K₃PO₄ (1.20 g, 3.0 equiv) and methyl piperidine-3-carboxylate (270 mg, 1.0 equiv). The reaction was stirred at 60 °C for 1 hour. The mixture was purified by reversed phase flash chromatography [C18, 0.1% formic acid condition] to afford the title compound (677 mg, 32% yield) as red solid; LCMS (ESI, M+1): m/z=574.3.

Step B. 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylic acid: To a solution of methyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylate (567 mg, 1.0 equiv) in EtOH (3 mL) and H₂O (1 mL) was added LiOH·H₂O (207 mg, 5.0 equiv). The reaction was stirred at 25 °C for 1 hour. The mixture was purified by reversed phase flash chromatography [C18, 0.1% formic acid condition] to afford the title compound (300 mg, 48% yield) as white solid; LCMS (ESI, M+1): m/z=560.2.

Step C. phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylic acid (100 mg, 1.0 equiv) in DCM (1 mL) were added phenol (25.2 mg, 1.5 equiv), EDCI (51.4 mg, 1.5 equiv) and DMAP (32.8 mg, 1.5 equiv). The reaction was stirred at 25 °C for 1 hour. The mixture was concentrated. The residue was purified by prep-HPLC [Phenomenex luna C18 150×25 mm×10 μm; A: water (FA), B: ACN, B %: 30%-60% over 7 min] to afford the title compound (20.9 mg, 33% yield, HCOOH salt) as white solid; ¹H NMR (400 MHz, DMSO-d₆) δ=9.18 (d, J=2.4 Hz, 1H), 8.19 (br d, J=8.0 Hz, 1H), 7.94 (d, J=8.0 Hz, 1H), 7.78-7.70 (m, 1H), 7.68-7.54 (m, 2H), 7.45-7.38 (m, 2H), 7.3-7.22 (m, 2H), 7.13-7.06 (m, 2H), 4.62-4.49 (m, 1H), 4.27-4.15 (m, 1H), 4.15-4.06 (m, 2H), 4.03-3.91 (m, 1H), 3.80-3.67 (m, 1H), 3.24-3.16 (m, 1H), 2.98-2.88 (m, 2H), 2.60-2.55 (m, 1H), 2.23 (br d, J=7.2 Hz, 1H), 2.08-1.70 (m, 10H), 1.64-1.53 (m, 2H); LCMS (ESI, M+1): m/z=636.4.

### Example 52

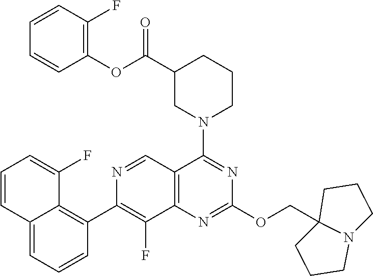 [SMILES: O=C(Oc1ccccc1F)C1CCCN(c2nc(OCC34CCCN3CCC4)nc3c(F)c(-c4cccc5cccc(F)c45)ncc23)C1]

### 2-fluorophenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylate

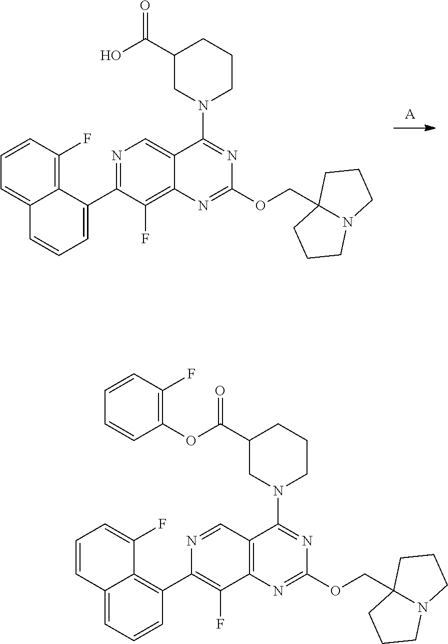

Step A. phenyl 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylate: To a solution of 1-(8-fluoro-7-(8-fluoronaphthalen-1-yl)-2-((tetrahydro-1H-pyrrolizin-7a(5H)-yl)methoxy)pyrido[4,3-d]pyrimidin-4-yl)piperidine-3-carboxylic acid (50 mg, 1.0 equiv) in DCM (0.5 mL) were added 2-fluorophenol (15.0 mg, 1.5 equiv), EDCI (25.7 mg, 1.5 equiv) and DMAP (16.4 mg, 1.5 equiv). The reaction was stirred at 25 °C for 1 hour. The mixture was concentrated. The residue was purified by prep-HPLC [Phenomenex luna C18 150×25 mm×10 μm; A: water (FA); B: ACN, B %: 30%-60% over 7 min] to afford the title compound (20.9 mg, 33% yield, HCOOH salt) as white solid; ¹H NMR (400 MHz, CHLOROFORM-d) δ=9.11 (d, J=4.4 Hz, 1H), 8.01 (br d, J=7.6 Hz, 1H), 7.82-7.71 (m, 1H), 7.68-7.58 (m, 2H), 7.46 (dt, J=4.8, 8.0 Hz, 1H), 7.25-7.10 (m, 5H), 4.7-4.62 (m, 1H), 4.59-4.48 (m, 2H), 4.46-4.32 (m, 1H), 4.05-3.86 (m, 1H), 3.77-3.60 (m, 1H), 3.51 (br d, J=4.0 Hz, 2H), 3.25-3.15 (m, 1H), 2.84-2.77 (m, 2H), 2.43-2.36 (m, 1H), 2.26 (br dd, J=6.0, 12.8 Hz, 2H), 2.14-1.91 (m, 7H), 1.88-1.77 (m, 2H); LCMS (ESI, M+1): m/z=654.4.

### Example 53

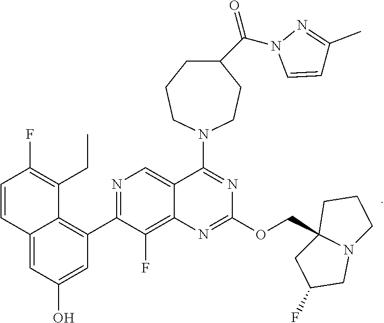 [SMILES: CCc1c(F)ccc2cc(O)cc(-c3ncc4c(N5CCCC(C(=O)n6ccc(C)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocan-4-yl)(3-methyl-1H-pyrazol-1-yl)methanone

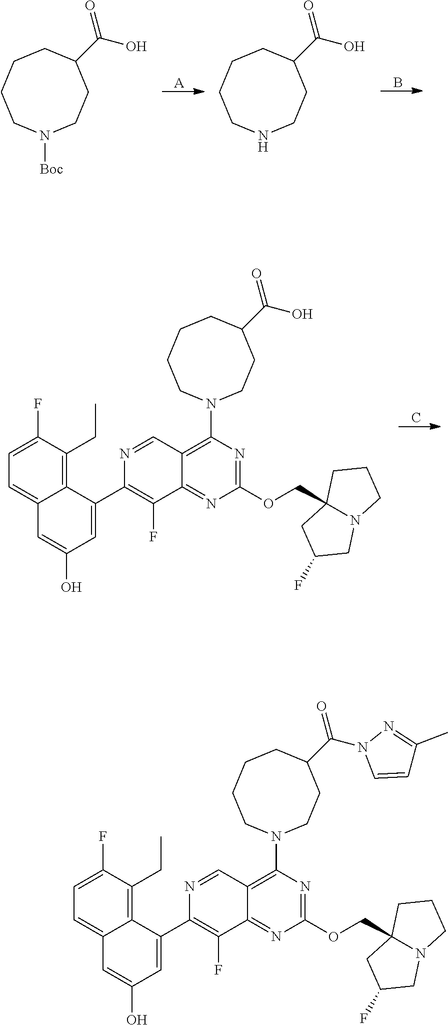

Step A. azocane-4-carboxylic acid: A solution of 1-(tert-butoxycarbonyl) azocane-4-carboxylic acid (300 mg, 1 equiv) in HCl·dioxane (2 M, 11.7 mL, 20 equiv) was stirred at 25 °C for 1 hour. The mixture was concentrated. The residue was diluted with methanol (2.0 mL) and neutralized with solid NaHCO₃. The mixture was filtered and the filtrate was concentrated to afford the title compound (180 mg, 88% yield) as yellow oil; ¹H NMR (400 MHz, METHANOL-d4) δ 3.39-3.32 (m, 1H), 3.27-3.10 (m, 3H), 2.77-2.64 (m, 1H), 2.15-2.06 (m, 2H), 1.99-1.85 (m, 4H), 1.80-1.67 (m, 2H).

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-4-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (280 mg, 1.0 equiv) and azocane-4-carboxylic acid (148 mg, 2.0 equiv) in DMF (2.5 mL) were added DIEA (244 mg, 4 equiv) and 4 Å molecular sieve (20 mg). The reaction was stirred at 60 °C for 2 hours. Azocane-4-carboxylic acid (150 mg, 1.6 equiv, HCl salt) and DIEA (244 mg, 4.0 equiv) were added to the resulting mixture. The reaction was stirred at 60 °C for 2 hours. The mixture was filtered and purified by reversed phase flash chromatography [water (0.1% FA)/acetonitrile] to afford the title compound (260 mg, 82% yield) as yellow solid; LCMS (ESI, M+1): m/z=650.4.

Step C. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocan-4-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-4-carboxylic acid (50 mg, 1 equiv) and EDCI (22.1 mg, 1.5 equiv) in DMF (0.5 mL) were added HOBt (12.5 mg, 1.2 equiv) and TEA (31.1 mg, 4.0 equiv). The reaction was stirred at 25 °C for 12 hours. The mixture was filtered and the filtrate was purified by prep-HPLC [Phenomenex Luna C18 150×25 mm×10 μm; A: water (NH₄HCO₃), B: ACN, B %: 56%-86% over 10 min] to afford the title compound (4.97 mg, 8.5% yield) as yellow solid; ¹H NMR (400 MHz, DMSO-d6) δ 9.97-9.88 (m, 1H), 9.21-9.16 (m, 1H), 8.25-8.19 (m, 1H), 7.81-7.71 (m, 1H), 7.38-7.29 (m, 2H), 6.98 (s, 1H), 6.44-6.38 (m, 1H), 5.37-5.13 (m, 1H), 4.35-4.07 (m, 4H), 4.05-3.97 (m, 2H), 3.91-3.81 (m, 1H), 3.11-2.95 (m, 3H), 2.87-2.75 (m, 1H), 2.31-2.25 (m, 1H), 2.16-2.00 (m, 9H), 1.98-1.92 (m, 1H), 1.91-1.65 (m, 8H), 0.76-0.65 (m, 3H); LCMS (ESI, M+1): m/z=714.5.

### Example 54

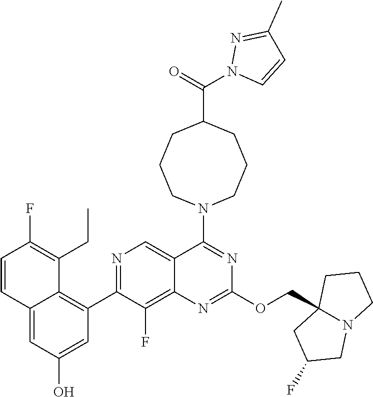 [SMILES: CCc1c(F)ccc2cc(O)cc(-c3ncc4c(N5CCCC(C(=O)n6ccc(C)n6)CCC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocan-5-yl)(3-methyl-1H-pyrazol-1-yl)methanone

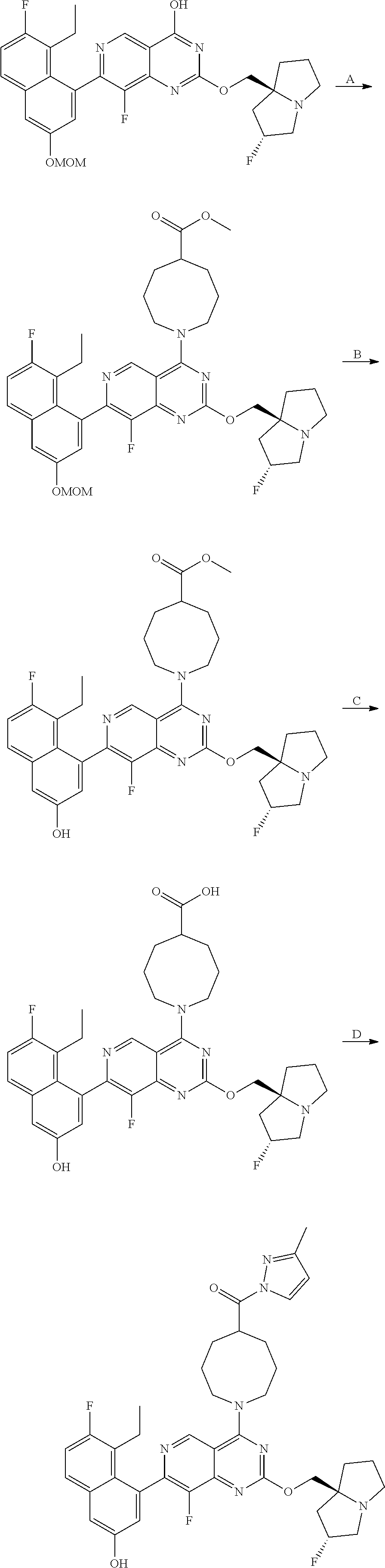

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-5-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (1 g, 1.0 equiv) and methyl azocane-5-carboxylate (309 mg, 1.0 equiv) in DMF (10 mL) were added DIEA (699 mg, 3.0 equiv) and PYBOP (1.41 g, 1.5 equiv). The reaction was stirred at 25 °C for 10 hours. The mixture was diluted with water (10 mL), extracted with ethyl acetate (2×20 mL). The combined organic layers were washed with brine (2×2 mL), dried over anhydrous sodium sulfate, concentrated and purified by reversed phase flash chromatography [C18, 0.1% formic acid condition] to afford the title compound (700 mg, 52% yield) as yellow solid; LCMS (ESI, M+1): m/z=708.4.

Step B. methyl 1-(7-(8-ethyl-7-fluoro-3-hydroxynaphthalen-1-yl)-8-fluoro-2-yl) azocane-5-carboxylate: To a solution of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-5-carboxylate (650 mg, 1.0 equiv) in MeOH (2 mL) was added HCl·MeOH (4 M, 2.00 mL, 8.7 equiv). The reaction was stirred at 0 °C for 0.5 hours. The mixture was concentrated. The residue was diluted with water (5 mL), neutralized with solid NaHCO₃ and extracted with ethyl acetate (2×20 mL). The combined organic layers were washed with brine (2×2 mL), dried over anhydrous sodium sulfate, concentrated to afford the title compound (600 mg, 86% yield) as yellow solid; LCMS (ESI, M+1): m/z=664.4.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-5-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-5-carboxylate (550 mg, 1.0 equiv) in MeOH (6 mL) were added LiOH·H₂O (2 M, 1.81 mL, 4.4 equiv). The reaction was stirred at 25 °C for 0.5 hours. The mixture was purified by reversed phase flash chromatography [C18, 0.1% formic acid condition] to afford the title compound (390 g, 72% yield) as white solid; LCMS (ESI, M+1): m/z=650.4.

Step D. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocan-5-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-5-carboxylic acid (60 mg, 1.0 equiv) and 3-methyl-1H-pyrazole (22.7 mg, 3.0 equiv) in DMF (0.5 mL) were added HATU (70.2 mg, 2.0 equiv) and DIEA (35.8 mg, 3.0 equiv). The reaction was stirred at 25 °C for 0.5 hours. The mixture was purified by reversed phase flash chromatography [C18, neutral condition] to afford the title compound (14.4 mg, 21% yield) as white solid; ¹H (400 MHz, CHLOROFORM-d) δ=9.08 (d, J=12.0 Hz, 1H), 8.13 (d, J=2.8 Hz, 1H), 7.58-7.48 (m, 1H), 7.22-7.07 (m, 3H), 6.25 (d, J=2.8 Hz, 1H), 5.39-5.17 (m, 1H), 4.23 (br d, J=3.2 Hz, 4H), 4.02-3.78 (m, 3H), 3.38-3.09 (m, 3H), 3.04-2.93 (m, 1H), 2.61-2.50 (m, 1H), 2.27 (s, 4H), 2.24-2.04 (m, 10H), 2.02-1.83 (m, 4H), 0.85 (br t, J=6.8 Hz, 3H); LCMS (ESI, M+1): m/z=714.5.

### Example 55

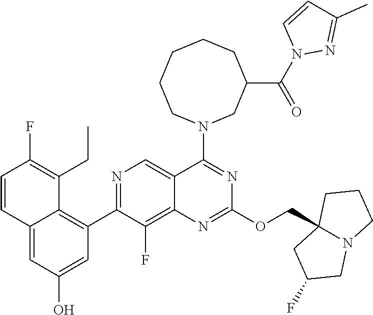 [SMILES: CCc1c(F)ccc2cc(O)cc(-c3ncc4c(N5CCCCCC(C(=O)n6ccc(C)n6)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocan-3-yl)(3-methyl-1H-pyrazol-1-yl)methanone

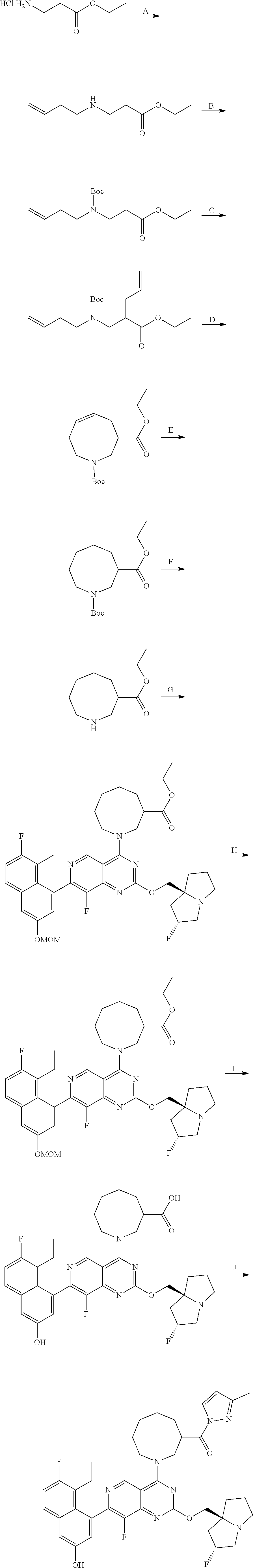

Step A. ethyl 3-(but-3-en-1-ylamino) propanoate: To a solution of ethyl 3-aminopropanoate hydrochloride (49.5 g, 1.5 equiv) in ACN (1000 mL) was added K₂CO₃ (87.5 g, 3.0 equiv). The reaction was stirred at 25 °C for 1 hour. 4-bromobut-1-ene (28.5 g, 1.0 equiv) was added. The reaction was stirred at 45 °C for 17 hours. The reaction was concentrated to afford the title compound (48.3 g, crude) as colorless oil; ¹H NMR (400 MHz, CHLOROFORM-d) δ=5.91-5.64 (m, 1H), 5.16-4.89 (m, 1H), 4.21-3.97 (m, 2H), 3.00-2.75 (m, 2H), 2.66 (t, J=6.8 Hz, 1H), 2.56-2.34 (m, 2H), 2.29-2.09 (m, 1H), 1.34-1.14 (m, 3H).

Step B. ethyl 3-(but-3-en-1-yl(tert-butoxycarbonyl)amino) propanoate: To a solution of ethyl 3-(but-3-enylamino) propanoate (43.3 g, 1.0 equiv) in DCM (450 mL) was added TEA (51.1 g, 2.0 equiv) and Boc₂O (82.7 g, 1.5 equiv). The reaction was stirred at 25 °C for 1 hour. The mixture was concentrated and purified by column chromatography (SiO₂, Petroleum ether/Ethyl acetate=1/0 to 20:1) to afford the title compound (40.2 g, 59% yield) as yellow oil.

Step C. ethyl 2-((but-3-en-1-yl(tert-butoxycarbonyl)amino)methyl) pent-4-enoate: A solution of ethyl 3-(but-3-en-1-yl(tert-butoxycarbonyl)amino) propanoate (20.0 g, 1.0 equiv) in THF (200 mL) was degassed and purged with nitrogen 3 times. LDA (2 M, 1.5 equiv) was added slowly at −65 °C, the reaction was stirred at −40 °C for 1 hour. 3-iodoprop-1-ene (13.6 g, 1.1 equiv) was added at −65 °C. The reaction was stirred at −40 °C for 0.5 hours. The mixture was quenched with water (100 mL) at 0 °C and extracted with ethyl acetate (3×100 mL). The combined organic layer was dried over anhydrous Na₂SO₄, filtered, concentrated and purified by column chromatography (SiO₂, Petroleum ether/Ethyl acetate=1/0 to 20:1) to afford the title compound (3.90 g, 15% yield) as yellow oil; ¹H NMR (400 MHz, CHLOROFORM-d) δ=5.81-5.64 (m, 2H), 5.11-4.95 (m, 4H), 4.16-4.06 (m, 2H), 3.50-3.21 (m, 3H), 3.17-3.05 (m, 1H), 2.95-2.68 (m, 1H), 2.39-2.11 (m, 4H), 1.44 (s, 9H), 1.23 (t, J=7.2 Hz, 3H).

Step D. 1-(tert-butyl) 3-ethyl (Z)-3,4,7,8-tetrahydroazocine-1,3 (2H)-dicarboxylate: To a solution of ethyl 2-((but-3-en-1-yl(tert-butoxycarbonyl)amino)methyl) pent-4-enoate (1.00 g, 1.0 equiv) in DCM (150 mL) was added Grubbs catalyst (265 mg, 0.10 equiv). The reaction was purged with nitrogen 3 times. The reaction was stirred at 40 °C for 1.5 hours. The mixture was filtered, concentrated and purified by column chromatography (SiO₂, Petroleum ether/Ethyl acetate=1/0 to 20/1) to afford the title compound (650 mg, 71% yield) as brown oil; ¹H NMR (400 MHz, CHLOROFORM-d) δ=5.96-5.59 (m, 2H), 4.20-4.06 (m, 3H), 3.91-3.75 (m, 1H), 3.42-3.05 (m, 1H), 3.01-2.89 (m, 1H), 2.83-2.58 (m, 1H), 2.49-2.21 (m, 3H), 2.19-2.06 (m, 1H), 1.47 (d, J=3.2 Hz, 9H), 1.26 (td, J=7.2, 10.3 Hz, 3H).

Step E. 1-(tert-butyl) 3-ethyl azocane-1,3-dicarboxylate: To a mixture of Pd/C (200 mg, 10% purity) in EtOH (20 mL) was added 1-(tert-butyl) 3-ethyl (Z)-3,4,7,8-tetrahydroazocine-1,3 (2H)-dicarboxylate (650 mg, 1.0 equiv). The suspension was degassed under vacuum and purged with H₂ several times. The reaction was stirred under H₂ (15 psi) at 25 °C for 1 hour. The mixture was filtered and concentrated to afford the title compound (381 mg, crude) as brown oil; ¹H NMR (400 MHz, CHLOROFORM-d) δ=4.20-4.05 (m, 2H), 3.94-3.60 (m, 2H), 3.16 (ddd, J=6.8, 11.2, 14.4 Hz, 1H), 3.03-2.80 (m, 2H), 1.98-1.82 (m, 1H), 1.77-1.59 (m, 5H), 1.49-1.42 (m, 10H), 1.29-1.20 (m, 4H).

Step F. ethyl azocane-3-carboxylate: A solution of 1-(tert-butyl) 3-ethyl azocane-1,3-dicarboxylate (381 mg, 1.0 equiv) in HCl·MeOH (2 M, 6.7 equiv) was stirred at 25 °C for 0.5 hours. The mixture was concentrated, diluted with MeOH (3 mL), adjusted pH to 8 with NaHCO₃solid, filtered and concentrated to afford the title compound (236 mg, crude) as yellow oil; ¹H NMR (400 MHz, CHLOROFORM-d) δ=4.18-3.99 (m, 2H), 3.31-3.22 (m, 1H), 3.19-3.13 (m, 2H), 3.02-2.89 (m, 2H), 2.17-2.06 (m, 1H), 1.96-1.81 (m, 4H), 1.68 (br s, 3H), 1.20 (t, J=7.2 Hz, 3H).

Step G. ethyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-3-carboxylate: To a solution 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 equiv), ethyl azocane-3-carboxylate (207 mg, 1.2 equiv) and DIEA (349 mg, 3.0 equiv) in DMF (5 mL) was added HATU (514 mg, 1.5 equiv). The reaction was stirred at 25 °C for 2 hours. The mixture was filtered, concentrated and purified by prep-HPLC [C18 150×30 mm; A: water (FA), B: ACN, B %: 35%-65% over 7 min] to afford the title compound (120 mg, 12%, HCOOH) as white solid; LCMS (ESI, M+1): m/z=722.4.

Step H. ethyl 1-(7-(8-ethyl-7-fluoro-3-hydroxynaphthalen-1-yl)-8-fluoro-2-yl) azocane-3-carboxylate: A solution of ethyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-3-carboxylate (170 mg, 1.0 equiv, FA) in HCl·MeOH (2 M, 45 equiv) was stirred at 25 °C for 1 hour. The mixture was concentrated, diluted with MeOH (3 mL), adjusted to pH about 8 with NaHCO₃solid, filtered and concentrated to afford the title compound (200 mg, crude) as yellow oil; LCMS (ESI, M+1): m/z=678.4.

Step I. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-3-carboxylic acid: To a solution of ethyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-3-carboxylate (180 mg, 1.0 equiv) in MeOH (5 mL) was added LiOH·H₂O (2 M, 38 equiv). The reaction was stirred at 25 °C for 0.5 hours. The mixture was filtered, concentrated and purified by prep-HPLC [C18 150×30 mm; A: water (FA), B: FA, B %: 25%-55% over 7 min] to afford the title compound (65.0 mg, 35%, HCOOH) as white solid.

Step J. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocan-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azocane-3-carboxylic acid (30.0 mg, 1.0 equiv, HCOOH) and 3-methyl-1H-pyrazole (10.6 mg, 3.0 equiv) in DMF (1 mL) was added DIEA (22.3 mg, 4.0 equiv). The reaction was stirred at 25 °C for 10 minutes. HATU (32.8 mg, 2.0 equiv) was added. The reaction was stirred at 25 °C for 0.5 hours. The mixture was filtered and purified by prep-HPLC [Waters Xbridge 150×25 mm×5 μm; A: water (NH₄HCO₃), B: ACN, B %: 58%-88% over 9 min] to afford the title compound (6.57 mg, 21%) as off-white solid; ¹H NMR (400 MHz, CHLOROFORM-d) δ=9.17-8.93 (m, 1H), 8.17 (t, J=3.2 Hz, 1H), 7.51 (td, J=5.6, 9.2 Hz, 1H), 7.13 (br s, 3H), 6.32-6.23 (m, 1H), 5.36-5.12 (m, 1H), 4.85-4.62 (m, 1H), 4.59-4.31 (m, 2H), 4.09-3.99 (m, 1H), 3.96-3.72 (m, 1H), 3.70-3.52 (m, 1H), 3.30-2.99 (m, 3H), 2.97-2.87 (m, 1H), 2.64-2.37 (m, 2H), 2.34-2.22 (m, 4H), 2.19-2.07 (m, 3H), 2.04-1.98 (m, 2H), 1.97-1.73 (m, 9H), 0.86-0.79 (m, 3H); LCMS (ESI, M+1): m/z=714.4.

### Example 56

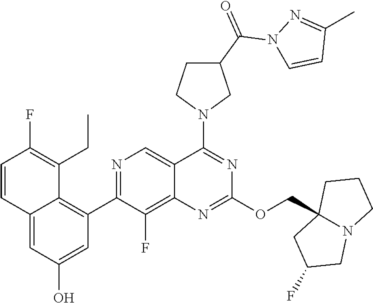 [SMILES: CCc1c(F)ccc2cc(O)cc(-c3ncc4c(N5CCC(C(=O)n6ccc(C)n6)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidin-3-yl)(3-methyl-1H -pyrazol-1-yl)methanone

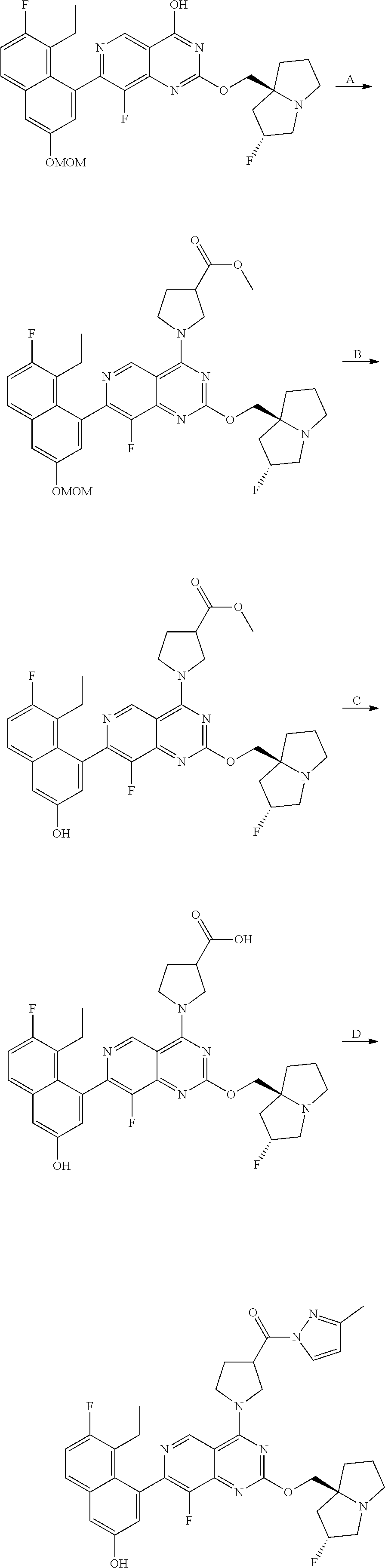

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidine-3-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (1.00 g, 1.0 equiv), PyBOP (1.41 g, 1.5 equiv) in DMF (10 mL) were added DIEA (1.40 g, 6.0 equiv) and methyl pyrrolidine-3-carboxylate (448 mg, 1.5 equiv, HCl). The reaction was stirred at 40 °C for 12 hours. The mixture was diluted with H₂O (20 mL) and extracted with ethyl acetate (3×10 mL). The combined organic layers were washed with brine (10 mL), dried over anhydrous sodium sulfate, concentrated and purified by reversed phase flash chromatography [C18, 0.1% formic acid condition] to afford the title compound (1.20 g, 98% yield) as yellow solid; ¹H NMR (400 MHz, DMSO-d₆) δ=9.30-9.22 (m, 1H), 7.93-7.85 (m, 1H), 7.69-7.64 (m, 1H), 7.46-7.39 (m, 1H), 7.27-7.10 (m, 4H), 5.39-5.19 (m, 3H), 4.24-3.89 (m, 6H), 3.69 (s, 3H), 3.45-3.41 (m, 4H), 3.14-3.08 (m, 2H), 3.07-3.06 (m, 1H), 2.89-2.79 (m, 1H), 2.44-2.31 (m, 2H), 2.29 (s, 2H), 2.19-2.11 (m, 2H), 2.09-1.98 (m, 2H), 1.91-1.75 (m, 4H); LCMS (ESI, M+1): m/z=666.4.

Step B. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidine-3-carboxylate: To a solution 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidine-3-carboxylate (1.10 g, 1.0 equiv) in MeOH (10 mL) was added HCl·MeOH (4 M, 24 equiv). The reaction was stirred at 25 °C for 12 hours. The mixture was concentrated, diluted with MeOH (10 mL), adjusted to pH about 8 with NaHCO₃solid, filtered and concentrated to afford the title compound (1.00 g, crude) as yellow solid.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidine-3-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidine-3-carboxylate (900 mg, 1.0 equiv) in MeOH (6 mL) and H₂O (2 mL) was added LiOH·H₂O (911 mg, 15 equiv). The reaction was stirred at 25 °C for 1 hour. The mixture was concentrated and adjust to pH=4 with HCl (12 M), filtered to afford the title compound (800 mg, 88% yield, HCl) as white solid; ¹H NMR (400 MHz, DMSO-d₆) δ=10.75-9.69 (m, 1H), 9.35-9.22 (m, 1H), 7.75 (dd, J=6.0, 8.8 Hz, 1H), 7.39-7.29 (m, 2H), 7.10-7.02 (m, 1H), 5.50-5.29 (m, 1H), 4.40-4.25 (m, 2H), 4.06-3.91 (m, 2H), 3.55-3.21 (m, 4H), 3.16 (s, 1H), 3.04-2.96 (m, 3H), 2.36-2.09 (m, 6H), 1.97-1.86 (m, 2H), 1.73-1.69 (m, 2H), 0.80-0.66 (m, 3H); LCMS (ESI, M+1): m/z=608.3.

Step D. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidin-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidine-3-carboxylic acid (200 mg, 1.0 equiv), EDCI (94.7 mg, 1.5 equiv) in DCM (2 mL) were added DMAP (60.3 mg, 1.5 equiv) and 3-methyl-1H-pyrazole (50.1 mg, 2.0 equiv). The reaction was stirred at 25 °C for 12 hours. The mixture was filtered and purified by prep-HPLC [Waters Xbridge Prep OBD C18 150×40 mm×10 μm; A: water (NH₄HCO₃), B: ACN, B %: 40%-70% over 20 min] to afford the title compound (42.3 mg, 18%) as yellow solid; ¹H NMR (400 MHz, CHLOROFORM-d) δ=9.18-9.02 (m, 1H), 8.21-8.10 (m, 1H), 7.48-7.41 (m, 1H), 7.15-7.08 (m, 1H), 7.07-7.02 (m, 1H), 6.99-6.83 (m, 1H), 6.33-6.27 (m, 1H), 5.39-5.15 (m, 1H), 4.41-4.17 (m, 5H), 4.11-3.87 (m, 2H), 3.42-3.24 (m, 2H), 3.23-3.12 (m, 1H), 2.99 (br d, J=5.6 Hz, 1H), 2.50-2.38 (m, 2H), 2.34 (s, 3H), 2.33-2.17 (m, 3H), 2.16-2.09 (m, 2H), 2.00-1.88 (m, 3H), 0.83-0.74 (m, 3H); LCMS (ESI, M+1): m/z=672.4.

### Example 57

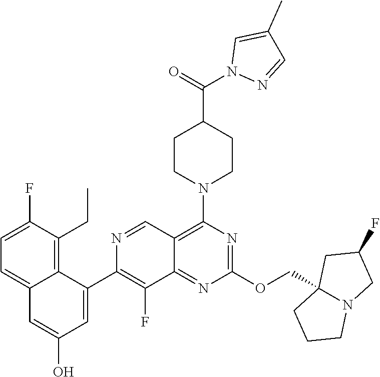 [SMILES: CCc1c(F)ccc2cc(O)cc(-c3ncc4c(N5CCC(C(=O)n6cc(C)cn6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(4-methyl-1H-pyrazol-1-yl)methanone

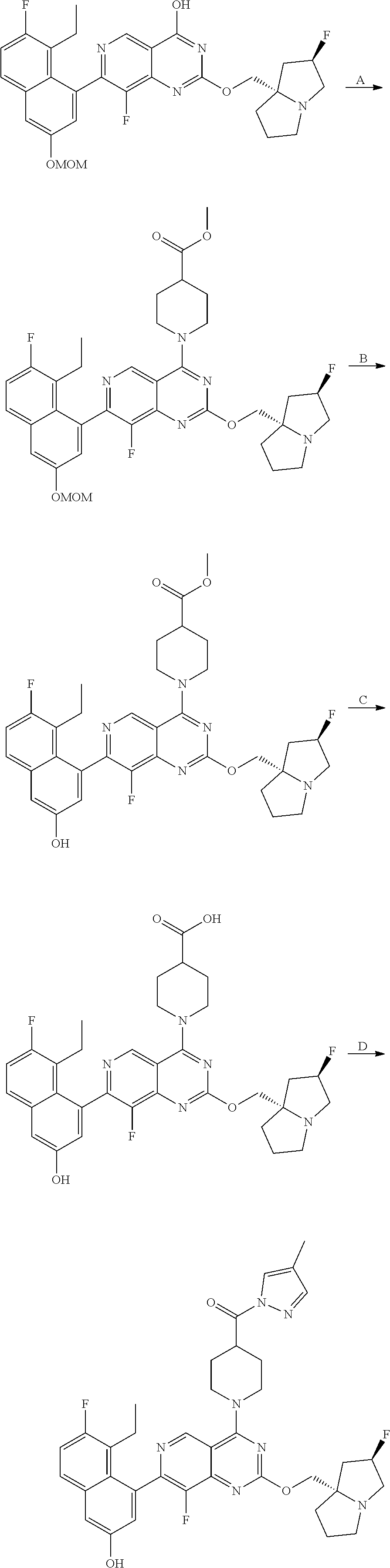

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (10 g, 1.0 equiv) and methyl piperidine-4-carboxylate (7.7 g, 3.0 equiv) in DMF (30 mL) were added DIEA (7.0 g, 3.0 equiv) and PyBOP (14.1 g, 1.5 equiv). The reaction was stirred at 25 °C for 25 hours. The mixture was diluted with H₂O (100 ml) and extracted with ethyl acetate (200 ml). The combined organic layers were washed with brine, dried over anhydrous sodium sulfate, concentrated to afford the title compound (10 g, 80% yield) as yellow solid; LCMS (ESI, M+1): m/z=680.4.

Step B. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: A solution of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (10 g, 1 equiv) and HCl·MeOH (2 M, 30 mL, 4.08 equiv) was stirred at 25 °C for 1 hour. The mixture was concentrated. The residue was basified with saturated NaHCO₃ solution (25 mL) and extracted with ethyl acetate (2×50 mL). The combined organic layers were washed with brine (2×2 mL), dried over anhydrous sodium sulfate, concentrated to afford the title compound (5 g, 41% yield) as yellow solid; LCMS (ESI, M+1): m/z=636.4.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (5 g, 1.0 equiv) in MeOH (15 mL) was added LiOH·H₂O (2 M, 15.7 mL, 4 equiv). The reaction was stirred at 25 °C for 1 hour. The mixture was purified by reversed phase flash chromatography [C18, 0.1% formic acid condition] to afford the title compound (3 g, 57% yield) as white solid; LCMS (ESI, M+1): m/z=622.4.

Step D. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(4-methyl-1H-pyrazol-1-yl)methanone

To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (200 mg, 1.0 equiv), DIEA (208 mg, 5.0 equiv), and HATU (147 mg, 1.2 equiv) in DMF (3.0 mL) was added 4-methyl-1H-pyrazole (79.2 mg, 3.0 equiv). The reaction was stirred at 30 °C for 2 hours. The mixture was filtered and purified by reversed phase flash [C18, H₂O condition], followed by [column: Phenomenex Luna 150×25 mm×10 μm; A: water (NH₄HCO₃), B: ACN; B %: 54%-84% over 10 min] to afford the title compound (11.0 mg, 4.8% yield) as a white solid; ¹H NMR (400 MHz, METHANOL-d4) δ=9.09-9.05 (m, 1H), 8.09 (s, 1H), 7.71-7.64 (m, 2H), 7.33-7.21 (m, 2H), 7.06 (d, J=2.4 Hz, 1H), 5.46-5.22 (m, 1H), 4.79-4.66 (m, 2H), 4.41-4.25 (m, 2H), 4.14-4.03 (m, 1H), 3.773.61 (m, 2H), 3.28-3.17 (m, 2H), 3.11-3.00 (m, 1H), 2.58-2.45 (m, 1H), 2.40-2.18 (m, 5H), 2.13 (s, 4H), 2.11-2.01 (m, 4H), 2.00-1.86 (m, 2H), 0.81 (br t, J=7.2 Hz, 3H); LCMS (ESI, M+1): m/z=686.4.

### Example 58

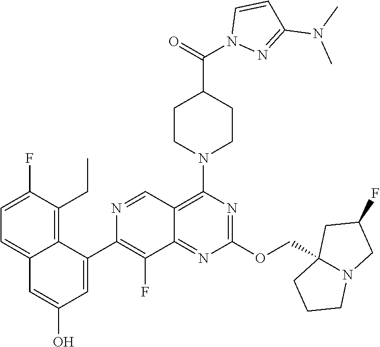 [SMILES: CCc1c(F)ccc2cc(O)cc(-c3ncc4c(N5CCC(C(=O)n6ccc(N(C)C)n6)CC5)nc(OC[C@@]56CCCN5C[C@H](F)C6)nc4c3F)c12]

### (3-(dimethylamino)-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone

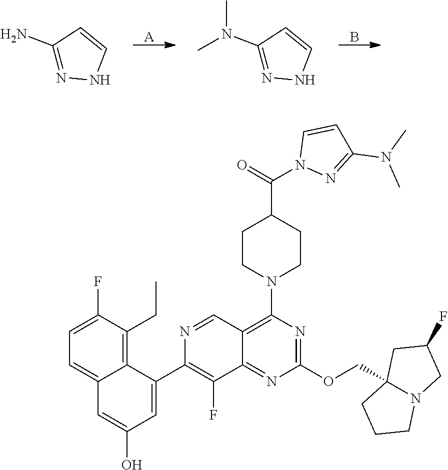

Step A. N,N-dimethyl-1H-pyrazol-3-amine: To a solution of 1H-pyrazol-3-amine (500 mg, 1.0 equiv) and formaldehyde (542 mg, 3.0 equiv) in MeOH (5 mL) were added AcOH (361 mg, 1.0 equiv). The reaction was stirred at 25 °C for 0.5 hours. NaBH₃CN (227 mg, 0.6 equiv) was added to the mixture. The reaction was stirred at 25 °C for 1 hour. The mixture was concentrated. The residue was diluted with water (10 mL), neutralized with solid NaHCO₃ and extracted with ethyl acetate (2×20 mL). The combined organic layers were washed with brine (2×5 mL), dried over anhydrous sodium sulfate, concentrated and purified by column chromatography (SiO₂, Petroleum ether/Ethyl acetate=10/1 to 0/1) to afford the title compound (150 mg, 19% yield) as colorless oil;

Step B. (3-(dimethylamino)-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (200 mg, 1.0 equiv) and N,N-dimethyl-1H-pyrazol-3-amine (53.6 mg, 1.5 equiv) in DMF (1 mL) were added HATU (245 mg, 2.0 equiv) and DIEA (125 mg, 3.0 equiv). The reaction was stirred at 25 °C for 0.5 hours. The mixture was purified by prep-HPLC [Waters Xbridge 150×25 mm×5 μm; A: water (ammonia hydroxide v/v)-ACN; B: ACN, B %: 45%-75% over 10 min] to afford the title compound (36.8 mg, 15% yield) as yellow solid; ¹H NMR (400 MHz, CHLOROFORM-d) δ=8.96 (d, J=2.4 Hz, 1H), 8.08 (d, J=3.2 Hz, 1H), 7.57-7.49 (m, 1H), 7.21-7.12 (m, 2H), 7.07-6.96 (m, 1H), 6.03 (d, J=3.2 Hz, 1H), 5.40-5.18 (m, 1H), 4.60 (br d, J=12.4 Hz, 2H), 4.33-4.22 (m, 2H), 3.98-3.85 (m, 1H), 3.57-3.42 (m, 2H), 3.38-3.12 (m, 3H), 2.98 (s, 6H), 2.55-2.42 (m, 1H), 2.40-2.04 (m, 9H), 2.00-1.87 (m, 3H), 0.83 (br t, J=7.6 Hz, 3H); LCMS (ESI, M+1): m/z=715.4.

### Example 59

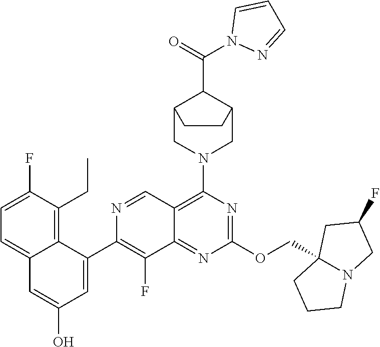 [SMILES: CCc1c(F)ccc2cc(O)cc(-c3ncc4c(N5CC6CCC(C5)C6C(=O)n5cccn5)nc(OC[C@@]56CCCN5C[C@H](F)C6)nc4c3F)c12]

### ((1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-8-yl)(1H-pyrazol-1-yl)methanone

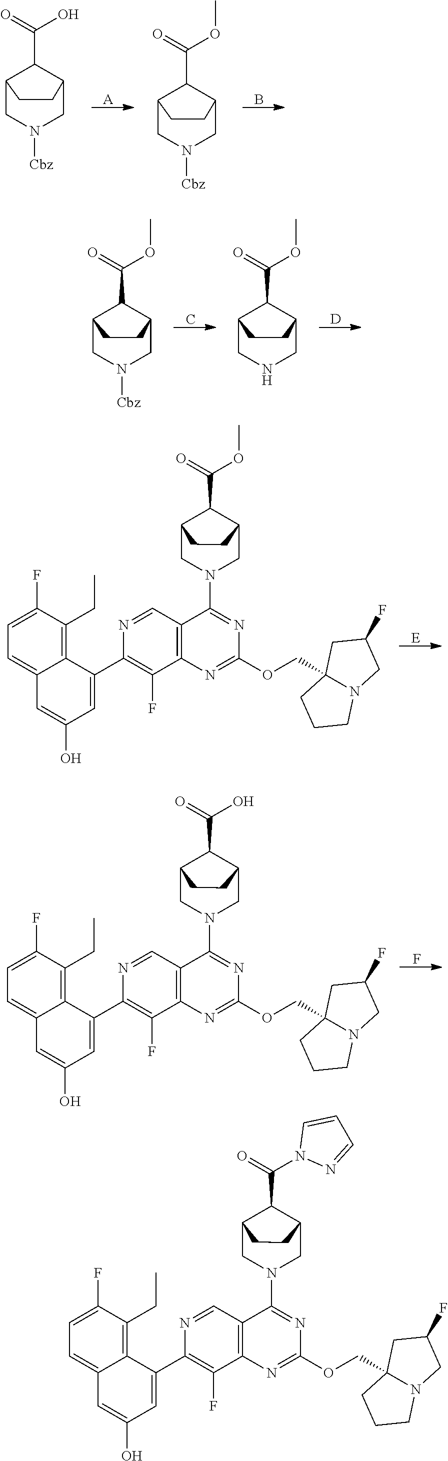

Step A. 3-benzyl 8-methyl (1R,5S,8r)-3-azabicyclo[3.2.1]octane-3,8-dicarboxylate: To a solution of (1R,5S,8r)-3-((benzyloxy)carbonyl)-3-azabicyclo[3.2.1]octane-8-carboxylic acid (1.15 g, 1.0 equiv) in DCM (10 mL) and MeOH (10 mL) was added TMSCHN₂ (2 M, 7.95 mL, 4.0 equiv) dropwise at 0 °C. The reaction was stirred at 20 °C for 3 hours. The mixture was quenched with CH₃COOH (10 mL) at 0 °C. The mixture concentrated and purified by column chromatography [SiO₂, petroleum ether/ethyl acetate=100/1 to 2/1] to afford the title compound (1.1 g, 44% yield, 96% purity) as colorless oil; LCMS (ESI, M+1): m/z=304.2.

Step B. 3-benzyl 8-methyl (1R,5S,8s)-3-azabicyclo[3.2.1]octane-3,8-dicarboxylate: 3-benzyl 8-methyl (1R,5S,8r)-3-azabicyclo[3.2.1]octane-3,8-dicarboxylate (1.1 g, 1.0 equiv) was purified by SFC [column: DAICEL CHIRALPAK IK (250 mm×30 mm, 10 μm); mobile phase: CO₂-i-PrOH; B %: 30%, isocratic elution mode] to afford two peaks.

Peak 1:3-benzyl 8-methyl (1R,5S,8s)-3-azabicyclo[3.2.1]octane-3,8-dicarboxylate (652 mg, crude) as colorless oil; SFC: Column: Chiralpak IC-3 50×4.6 mm I.D., 3umMobile phase: Phase A for CO₂, and Phase B for IPA (0.05% DEA); Gradient elution: IPA (0.05% DEA) in CO₂ from 5% to 40% Flow rate: 3 mL/min; Detector: PDA; Column Temp: 35C; Back Pressure: 100Bar. t_{R}: 1.533 min.

Peak 2:3-benzyl 8-methyl (1R,5S,8r)-3-azabicyclo[3.2.1]octane-3,8-dicarboxylate (400 mg, crude, RT=1.773 min) as colorless oil; t_{R}: 1.777 min.

Step C. methyl (1R,5S,8r)-3-azabicyclo[3.2.1]octane-8-carboxylate: To a solution of 3-benzyl 8-methyl (1R,5S,8s)-3-azabicyclo[3.2.1]octane-3,8-dicarboxylate (652 mg, 1.0 equiv) in MeOH (10 mL) was added Pd/C (100 mg, 10% purity) under N₂ atmosphere. The reaction was degassed and purged with H₂ for 3 times. The reaction stirred at 20 °C for 2 hours under H₂ (15 psi). The mixture was filtered through a pad of Celite and concentrated to afford the title compound (310 mg, crude) as colorless oil.

Step D. methyl (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-8-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and methyl (1R,5S,8r)-3-azabicyclo[3.2.1]octane-8-carboxylate (128 mg, 1.5 equiv) in DMAc (1 mL) was added DIEA (261 mg, 4.0 equiv). The reaction was stirred at 80 °C for 2 hours. The mixture was purified by reversed phase flash [0.1% FA condition] to afford the title compound (275 mg, 61% yield) as off-white solid; LCMS (ESI, M+1): m/z=662.3.

Step E. (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-8-carboxylic acid: To a solution of methyl (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-8-carboxylate (255 mg, 1.0 equiv) in MeOH (1 mL) was added LiOH·H₂O (2 M in H₂O, 433 μL, 3.0 equiv). The mixture was stirred at 20 °C for 2 hours. The pH of the mixture was adjusted to 8 with 1N HCl at 0 °C. The mixture was purified by reversed phase flash [0.1% FA condition] to afford the title compound (180 mg, 70% yield) as off-white solid; LCMS (ESI, M+1): m/z=648.4.

Step F. ((1R,5S,8S)-3-(7-(8-ethyl-7-fluoro-3-hydroxynaphthalen-1-yl)-8-fluoro-2-yl)-3-azabicyclo[3.2.1]octan-8-yl)(1H-pyrazol-1-yl)methanone: To a solution of (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-8-carboxylic acid (60 mg, 1.0 equiv) and 1H-pyrazole (50 mg, 8.0 equiv) in DMF (1 mL) were added TEA (75 mg, 8.0 equiv), HOBt (19 mg, 1.5 equiv) and EDCI (27 mg, 1.5 equiv). The reaction was stirred at 40 °C for 12 hours. The mixture was purified by prep-HPLC [column: Waters Xbridge 150×25 mm×5 μm; mobile phase: water (NH₄HCO₃)-ACN; gradient: 50%-80% B over 53 minutes] to afford the title compound (9.1 mg, 13.6% yield) as white solid; ¹H NMR (400 MHz, CD₃OD) δ=9.11 (s, 1H), 8.35 (d, J=2.8 Hz, 1H), 7.84 (d, J=1.2 Hz, 1H), 7.68 (dd, J=6.0, 9.2 Hz, 1H), 7.30 (d, J=2.8 Hz, 1H), 7.25 (t, J=9.2 Hz, 1H), 7.08-7.03 (m, 1H), 6.56 (dd, J=1.6, 2.8 Hz, 1H), 5.40-5.23 (m, 1H), 4.40-4.21 (m, 2H), 4.15-4.02 (m, 1H), 3.99-3.62 (m, 2H), 3.29-3.12 (m, 3H), 3.07-2.96 (m, 1H), 2.93-2.76 (m, 2H), 2.61-2.09 (m, 6H), 2.08-1.75 (m, 6H), 1.74-1.58 (m, 2H), 0.80 (dt, J=2.0, 7.2 Hz, 3H); LCMS (ESI, M+1): m/z=698.4.

### Example 60

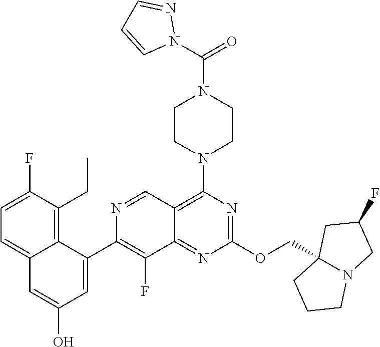 [SMILES: CCc1c(F)ccc2cc(O)cc(-c3ncc4c(N5CCN(C(=O)n6cccn6)CC5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) piperazin-1-yl)(1H-pyrazol-1-yl) methanone

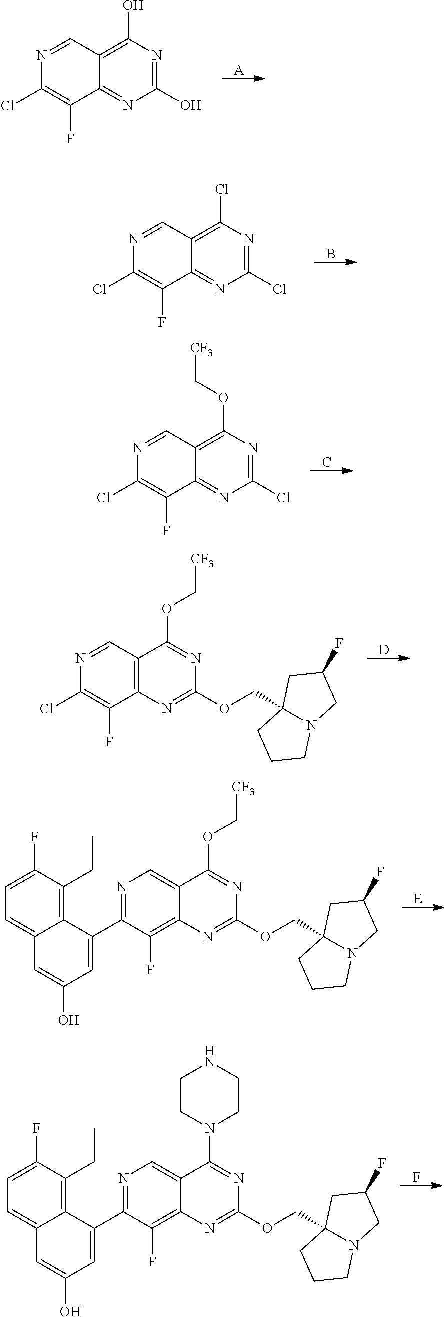

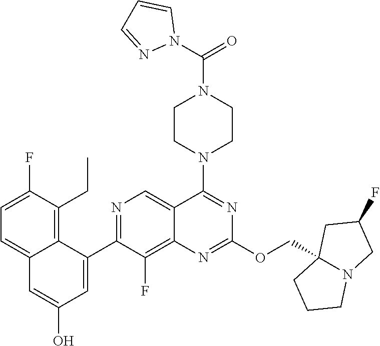 [SMILES: CCc1c(F)ccc2cc(O)cc(-c3ncc4c(N5CCN(C(=O)n6cccn6)CC5)nc(OC[C@@]56CCCN5C[C@H](F)C6)nc4c3F)c12]

Step A. 2,4,7-trichloro-8-fluoropyrido[4,3-d]pyrimidine: To a solution of 7-chloro-8-fluoro-pyrido[4,3-d]pyrimidine-2,4-diol (100 g, 1.0 equiv) in toluene (300 mL) were added POCl₃ (356 g, 5.0 equiv) and DIEA (132 g, 2.2 equiv) at −40 °C. The reaction was stirred at 110 °C for 12 hours. The mixture was concentrated under reduced pressure to give a residue. The residue was quenched by addition saturated NaHCO₃ (2000 mL) at 0 °C. The mixture was filtered and the filter cake was dissolved in ethyl acetate (1000 mL). The organic phase was washed with saturated NaHCO₃ (1000 mL), dried over Na₂SO₄, filtered and concentrated under reduced pressure to give a residue. The crude product was triturated with petroleum ether (500 mL) at 25 °C for 30 min to afford the title compound (50 g, 81% yield) as yellow solid.

Step B. 2,7-dichloro-8-fluoro-4-(2,2,2-trifluoroethoxy)pyrido[4,3-d]pyrimidine: To a solution of 2,2,2-trifluoroethanol (52 g, 1.1 equiv) in THF (200 mL) was added t-BuONa (2 M, 261 mL, 1.1 equiv) at 25 °C. The reaction was stirred at 25 °C for 1 hour. The mixture was added to a solution of 2,4,7-trichloro-8-fluoro-pyrido[4,3-d]pyrimidine (120 g, 1.0 equiv) in THF (1200 mL) at −40 °C. The reaction was stirred at 0 °C for 1 hour. The mixture was quenched with H₂O (500 mL) and extracted with ethyl acetate (3×300 mL). The combined organic layers were washed with brine (400 mL), dried over anhydrous sodium sulfate, concentrated. The crude product was triturated with n-heptane (200 ml) and MTBE (20 mL) at 25 °C for 60 min to afford the title compound (120 g, crude) as yellow solid; LCMS (ESI, M+1): m/z=315.9.

Step C. 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: To a solution of 2,7-dichloro-8-fluoro-4-(2,2,2-trifluoroethoxy)pyrido[4,3-d]pyrimidine (120 g, 1.0 equiv) and [(2R,8S)-2-fluoro-1,2,3,5,6,7-hexahydropyrrolizin-8-yl]methanol (66.5 g, 1.1 equiv) in THF (1200 mL) was added Na₂CO₃ (121 g, 3.0 equiv). The reaction was stirred at 40 °C for 12 hours. The mixture was filtered and purified by reversed phase flash chromatography [water (FA, 0.1%)/acetonitrile] to afford the title compound (100 g, two steps: 57% yield) as yellow solid; LCMS (ESI, M+1): m/z=439.1.

Step D. 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (28.4 g, 1.0 equiv), 5-ethyl-6-fluoro-4-(4,4,5,5-tetramethyl-1,3,2-dioxaborolan-2-yl)naphthalen-2-ol (25.6 g, 1.25 equiv) and Cs₂CO₃ (1.5 M in H₂O, 129 mL, 3.0 equiv) in methoxycyclopentane (300 mL) was added AdenBuP-Pd-G3 (7.07 g, 0.15 equiv). The reaction was degassed and purged with nitrogen 3 times. The reaction was stirred at 100 °C for 3 hours. The mixture was diluted with water (300 mL) and extracted with ethyl acetate (3×200 mL). The combined organic layers were dried over anhydrous sodium sulfate, concentrated and purified by reversed phase flash chromatography [water (0.1% FA)/acetonitrile] to afford the title compound (16.8 g, 40% yield) as yellow solid; ¹H NMR (400 MHz, chloroform-d) δ=9.19 (d, J=10.4 Hz, 1H), 7.54 (dd, J=5.6, 8.8 Hz, 1H), 7.26-7.21 (m, 1H), 7.16-7.11 (m, 1H), 6.96-6.84 (m, 1H), 5.46-5.22 (m, 1H), 5.05-4.71 (m, 2H), 4.56-4.32 (m, 2H), 3.48-3.21 (m, 3H), 3.13-3.01 (m, 1H), 2.47-2.32 (m, 4H), 2.32-2.08 (m, 4H), 0.78 (t, J=7.2 Hz, 3H); LCMS (ESI, M+1): m/z=593.2.

Step E 5-ethyl-6-fluoro-4-(8-fluoro-2-(((2R,7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol: To a solution of piperazine (727 mg, 5.0 equiv) in DMF (10 mL) was added 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (1.00 g, 1.0 equiv). The reaction was stirred at 25 °C for 2 hours. The mixture was purified by reversed phase flash chromatography [water (neutral)/acetonitrile=1/1] to afford the title compound (914 mg, 90% yield) as yellow solid; LCMS (ESI, M+1): m/z=579.4.

Step F. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) piperazin-1-yl)(1H-pyrazol-1-yl)methanone: To a mixture of 1H-pyrazole (20.0 mg, 1.0 equiv), DIEA (151 mg, 4.0 equiv) in DCM (0.3 mL) was added bis(trichloromethyl) carbonate (120 mg, 1.4 equiv) at 0 °C. The reaction was stirred at 0 °C for 1 hour. 5-ethyl-6-fluoro-4-(8-fluoro-2-(((2R,7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (153 mg, 0.9 equiv) and DIEA (56.9 mg, 1.5 equiv) in DCM (0.7 mL) were added dropwise into the mixture at 0 °C. The reaction was stirred at 20 °C for 14 hours. The mixture was quenched with saturated NaHCO₃ aqueous (5 mL) and extracted with DCM (4×3 mL). The combined organic layers were washed with brine (5 mL), dried over anhydrous sodium sulfate, concentrated and purified by prep-HPLC [column: Waters Xbridge 150×25 mm×5 μm; mobile phase: water (ammonia hydroxide v/v)—acetonitrile; gradient: 42%-72% B over 10 minutes] to afford the title compound (75.7 mg, 38% yield) as white solid; ¹H NMR (400 MHz, DMSO-d6) δ=9.95 (br s, 1H), 9.18 (s, 1H), 8.28 (d, J=2.4 Hz, 1H), 7.82 (s, 1H), 7.77 (dd, J=6.0, 9.0 Hz, 1H), 7.40-7.30 (m, 2H), 7.02 (d, J=1.6 Hz, 1H), 6.53 (br d, J=1.6 Hz, 1H), 5.39-5.17 (m, 1H), 4.24-3.97 (m, 10H), 3.12-3.03 (m, 2H), 3.02-2.90 (m, 1H), 2.85-2.79 (m, 1H), 2.42-2.30 (m, 1H), 2.20-2.09 (m, 2H), 2.08-1.95 (m, 2H), 1.89-1.71 (m, 3H), 0.73 (br t, J=7.2 Hz, 3H); LCMS (ESI, M+1): m/z=673.2.

### Example 61

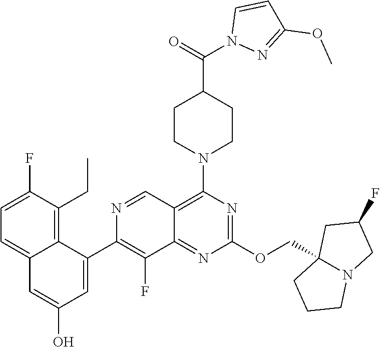 [SMILES: CCc1c(F)ccc2cc(O)cc(-c3ncc4c(N5CCC(C(=O)n6ccc(OC)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-methoxy-1H-pyrazol-1-yl)methanone

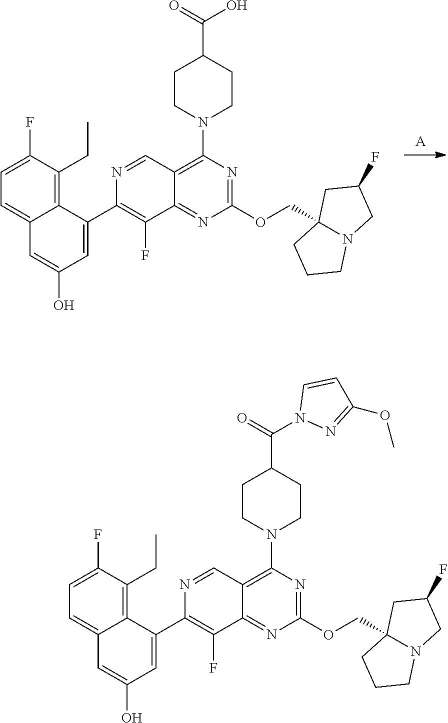

StepA. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-methoxy-1H-pyrazol-1-yl)methanone; To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (350 mg, 1.0 equiv) and 3-methoxy-1H-pyrazole (55.2 mg, 1.0 equiv) in DMF (4 mL) were added HATU (428 mg, 2.0 equiv) and DIEA (364 mg, 5.0 equiv). The reaction was stirred at 25 °C for 3 hours. The mixture was diluted with water (4 mL) and extracted with ethyl acetate (4 mL). The organic layer was dried over sodium sulfate, filtered and concentrated and purified by prep-HPLC [Waters Xbridge 150×25 mm×5 μm; A: water (NH₄HCO₃), B: ACN, B %: 54%-84% over 9 min] to afford the title compound (15.5 mg, 4% yield) as yellow solid. ¹H NMR (400 MHz, CHLOROFORM-d) δ=8.97 (s, 1H), 8.08 (d, J=3.2 Hz, 1H), 7.60-7.51 (m, 1H), 7.23-7.16 (m, 2H), 7.06-6.97 (m, 1H), 6.02 (d, J=2.8 Hz, 1H), 5.35 (br s, 1H), 4.69-4.55 (m, 2H), 4.29 (br d, J=6.4 Hz, 2H), 3.99 (s, 3H), 3.90 (br dd, J=5.2, 10.1 Hz, 1H), 3.59-3.43 (m, 2H), 3.36-3.14 (m, 3H), 3.05 (s, 1H), 2.56-2.40 (m, 2H), 2.27-2.06 (m, 7H), 1.99-1.87 (m, 3H), 0.83 (br t, J=7.2 Hz, 3H) LCMS (ESI, M+1): m/z=702.4.

### Example 62

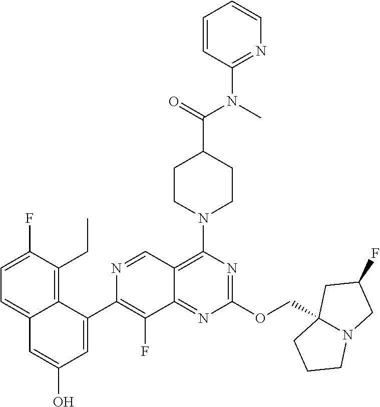 [SMILES: CCc1c(F)ccc2cc(O)cc(-c3ncc4c(N5CCC(C(=O)N(C)c6ccccn6)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(pyridin-2-yl)piperidine-4-carboxamide

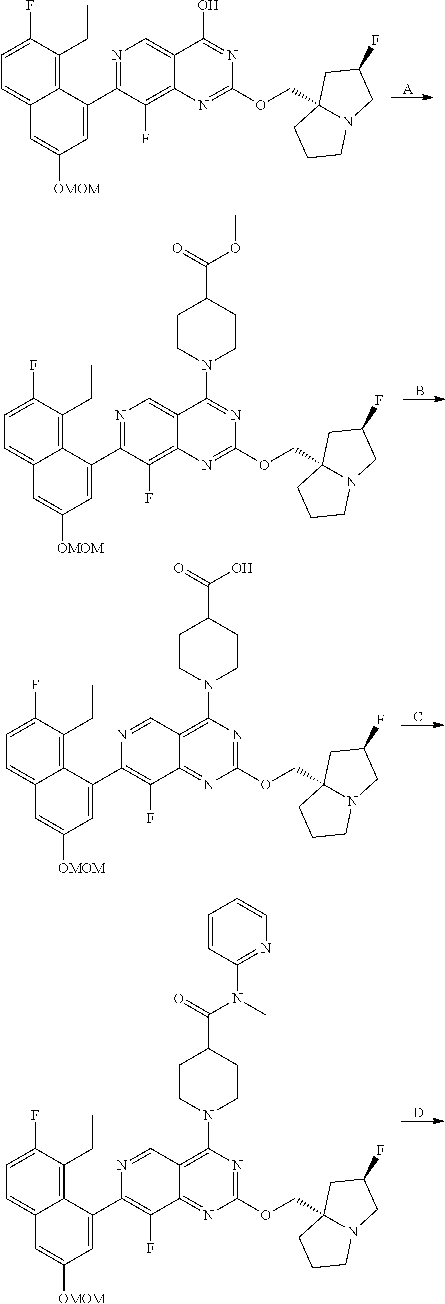

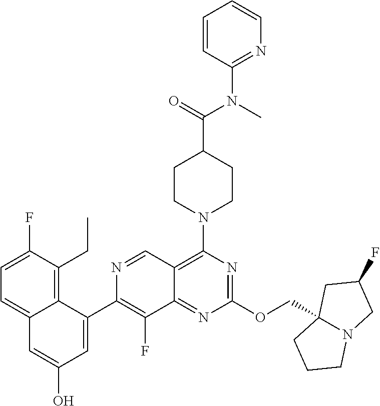 [SMILES: CCc1c(F)ccc2cc(O)cc(-c3ncc4c(N5CCC(C(=O)N(C)c6ccccn6)CC5)nc(OC[C@@]56CCCN5C[C@H](F)C6)nc4c3F)c12]

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: e: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (2.00 g, 1.0 equiv) and methyl piperidine-4-carboxylate (1.03 g, 2.0 equiv) in DMF (20 mL) were added PyBOP (2.82 g, 1.5 equiv) and DIEA (1.40 g, 3.0 equiv). The reaction was stirred at 25 °C for 3 hours. The mixture was diluted with water (100 mL) and extracted with ethyl acetate (3×50 mL). The organic layers were dried over anhydrous sodium sulfate, concentrated and purified by reversed phase flash chromatography [C18, 0.1% formic acid condition] to afford the title compound (2.1 g, 74% yield) as yellow solid; LCMS (ESI, M+1): m/z=680.3.

Step B. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid: To a solution of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (2.0 g, 1.0 equiv) in MeOH (6.2 mL) was added LiOH·H₂O (2 M in H₂O, 6.2 mL, 4.2 equiv). The reaction was stirred at 25 °C for 0.5 hours. The mixture was diluted with water (10 mL) and extracted with ethyl acetate (3×5 mL). The organic layers were dried over anhydrous sodium sulfate, concentrated and purified by reversed phase flash chromatography [C18, 0.1% formic acid condition] to afford the title compound (600 mg, 30% yield) as white solid; LCMS (ESI, M+1): m/z=666.4.

Step C. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(pyridin-2-yl)piperidine-4-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (300 mg, 1.0 equiv) and N-methylpyridin-2-amine (195 mg, 4.0 equiv) in THF (3 mL) were added 2-Chloro-1-Methylpyridinium Iodide (460 mg, 4.0 equiv) and DIEA (349 mg, 6.0 equiv). The reaction was stirred at 25 °C for 12 hours. The mixture was diluted with water (5 mL) and extracted with ethyl acetate (3×5 mL). The organic layers were dried over anhydrous sodium sulfate, concentrated and purified by prep-HPLC [Waters Xbridge 150×25 mm×5 μm; A: water (ammonia hydroxide), B: ACN, B %: 40%-70% over 10 min] to afford the title compound (112 mg, 32% yield) as yellow solid; LCMS (ESI, M+1): m/z=756.4.

Step D. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(pyridin-2-yl)piperidine-4-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(pyridin-2-yl)piperidine-4-carboxamide (112 mg, 1.0 equiv) in MeOH (1 mL) was added HCl·MeOH (4 M, 1 mL, 27 equiv). The reaction was stirred at 25 °C for 0.5 hours. The mixture was concentrated, basified with saturated NaHCO₃ solution (6 mL) and extracted with ethyl acetate (3×5 mL). The combined organic layers were dried over anhydrous sodium sulfate, concentrated and purified by prep-HPLC [Waters Xbridge 150×25 mm×5 μm; A: water (FA), B: ACN, B %: 15%-45% over 10 min] to afford the title compound (71.4 mg, 67% yield, 0.23 HCOOH) as yellow solid; ¹H NMR (400 MHz, CHLOROFORM-d) δ=8.86 (s, 1H), 8.56-8.50 (m, 1H), 7.82 (br t, J=7.8 Hz, 1H), 7.52-7.43 (m, 1H), 7.32-7.27 (m, 1H), 7.24 (s, 1H), 7.17-7.08 (m, 2H), 7.02-6.90 (m, 1H), 5.42-5.16 (m, 1H), 4.59-4.41 (m, 2H), 4.39-4.21 (m, 2H), 3.52 (br d, J=14.0 Hz, 1H), 3.36 (s, 3H), 3.33-2.94 (m, 5H), 2.86-2.71 (m, 1H), 2.48-1.83 (m, 12H), 0.77 (br t, J=7.2 Hz, 3H); LCMS (ESI, M+1): m/z=712.4.

### Example 63

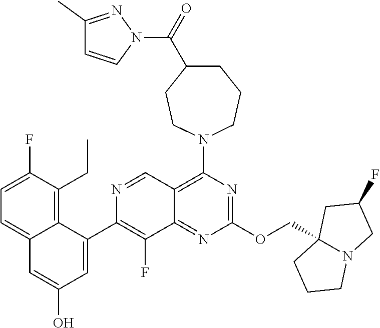 [SMILES: CCc1c(F)ccc2cc(O)cc(-c3ncc4c(N5CCCC(C(=O)n6ccc(C)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepan-4-yl)(3-methyl-1H-pyrazol-1-yl)methanone

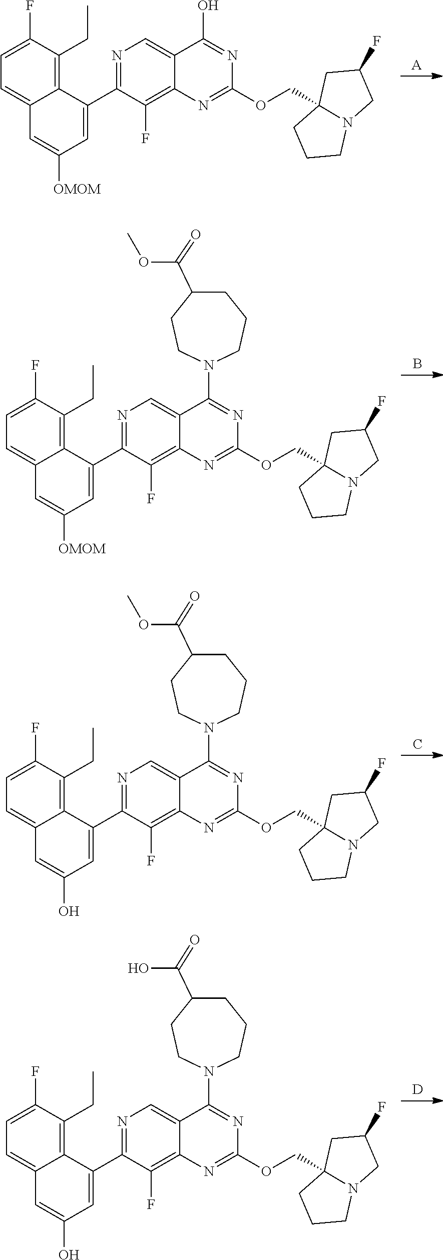

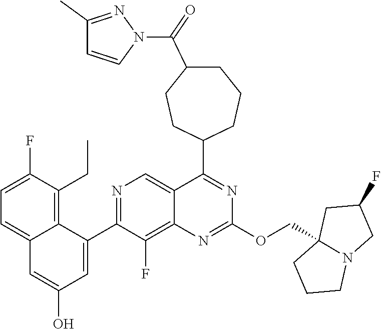 [SMILES: CCc1c(F)ccc2cc(O)cc(-c3ncc4c(C5CCCC(C(=O)n6ccc(C)n6)CC5)nc(OC[C@@]56CCCN5C[C@H](F)C6)nc4c3F)c12]

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-4-carboxylate: To a mixture of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (400 mg, 1.0 equiv) and benzotriazol-1-yloxyl-tris-(pyrrolidino)-phosphonium hexafluorophosphate (751 mg, 2.0 equiv) in DMSO (4 mL) were added TEA (301 μL, 3.0 equiv) and methyl azepane-4-carboxylate (340 mg, 3.0 equiv). The reaction was stirred at 35 °C for 12 hours. The mixture was diluted with water (20 mL) and extracted with ethyl acetate (2×20 mL). The combined organic layers were washed with brine (15 mL), dried over anhydrous sodium sulfate, concentrated and purified by reversed phase flash [C18, 0.1% formic acid condition] to afford the title compound (380 mg, 76% yield) as yellow oil; LCMS (ESI, M+1): m/z=694.4.

Step B. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-4-carboxylate: To a solution of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-4-carboxylate (260 mg, 1.0 equiv) in ACN (2 mL) was added HCl·dioxane (4 M, 3.5 mL, 37 equiv) at 0 °C. The reaction was stirred at 15 °C for 1 hour. The mixture was adjusted to pH=7 with saturated NaHCO₃ (10 mL) and extracted with ethyl acetate (2×10 mL). The combined organic layers were dried over anhydrous sodium sulfate, concentrated to afford the title compound (300 mg, crude) as yellow oil.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-4-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-4-carboxylate (300 mg, 1.0 equiv) in MeOH (3 mL) was added LiOH (2 M, 923 μL, 4.0 equiv). The reaction was stirred at 15 °C for 2 hours. The mixture was adjusted to pH=8 with HCl (1 M) and purified by reversed phase flash [C18, 0.1% formic acid condition] to afford the title compound (190 mg, 64% yield) as white solid; LCMS (ESI, M+1): m/z=636.4.

Step D. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepan-4-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-4-carboxylic acid (30.0 mg, 1.0 equiv) and 3-methyl-1H-pyrazole (7.75 mg, 2.0 equiv) in DMF (0.3 mL) were added HATU (35.9 mg, 2.0 equiv) and TEA (19.7 μL, 3.0 equiv). The reaction was stirred at 25 °C for 1 hour. The mixture was diluted with water (10 mL) and extracted with ethyl acetate (2×10 mL). The combined organic layers were washed with brine (10 mL), dried over anhydrous sodium sulfate, concentrated and purified by prep-HPLC [column: Waters xbridge 150×25 mm×10 μm; A: water (NH₄HCO₃), B: ACN; B %: 53%-73% over 8 min] to afford the title compound (9.07 mg, 27% yield) as white solid; ¹H NMR (400 MHz, METHANOL-d4) δ=9.18 (d, J=2.4 Hz, 1H), 8.19 (t, J=3.2 Hz, 1H), 7.68 (dd, J=6.0, 9.2 Hz, 1H), 7.34-7.19 (m, 2H), 7.07 (dd, J=2.4, 7.6 Hz, 1H), 6.37 (d, J=1.2 Hz, 1H), 5.37-5.20 (m, 1H), 4.49-4.22 (m, 4H), 4.14 (br s, 1H), 4.02 (br d, J=9.2 Hz, 1H), 3.88 (br s, 1H), 3.21 (br d, J=19.8 Hz, 3H), 3.08-2.96 (m, 1H), 2.57-2.41 (m, 2H), 2.39-2.31 (m, 1H), 2.31-2.10 (m, 10H), 2.05-1.94 (m, 2H), 1.92-1.77 (m, 2H), 0.87-0.77 (m, 3H); LCMS (ESI, M+1): m/z=700.4.

### Example 64

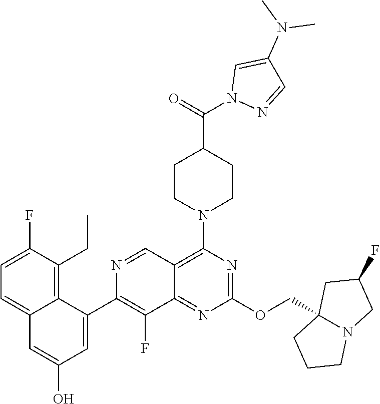 [SMILES: CCc1c(F)ccc2cc(O)cc(-c3ncc4c(N5CCC(C(=O)n6cc(N(C)C)cn6)CC5)nc(OC[C@@]56CCCN5C[C@H](F)C6)nc4c3F)c12]

### (4-(dimethylamino)-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone

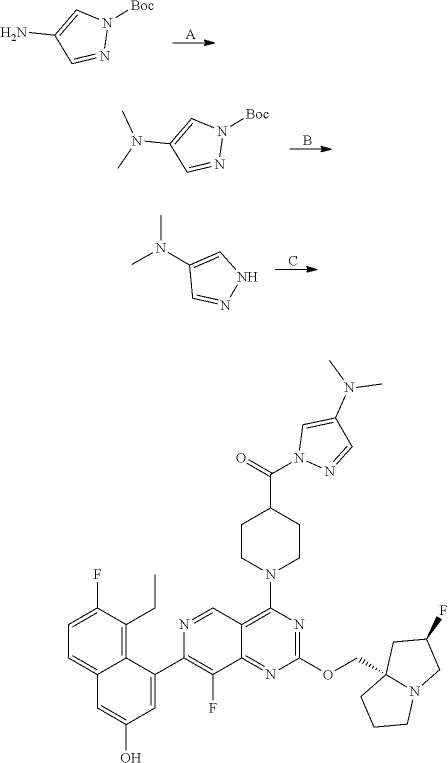

Step A. tert-butyl 4-(dimethylamino)-1H-pyrazole-1-carboxylate: To a solution of tert-butyl 4-aminopyrazole-1-carboxylate (500 mg, 1.0 equiv) and formaldehyde (2.22 g, 37% purity, 10 equiv) in MeOH (10 mL) were added AcOH (164 mg, 1.0 equiv). The reaction was stirred at 25 °C for 0.5 hours. Sodium cyanoborohydride (514 mg, 3.0 equiv) was added to the reaction mixture. The reaction was stirred at 25 °C for 0.5 hours. The mixture was concentrated, basified with NaHCO₃ (10 mL) and extracted with ethyl acetate (2×30 mL). The combined organic layers were washed with brine (2×2 mL), dried over anhydrous sodium sulfate, concentrated and purified by column chromatography (SiO₂, Petroleum ether/Ethyl acetate=10/1 to 3/1) to afford the title compound (500 mg, 81% yield) as white oil; LCMS (ESI, M−99, M−55): m/z=112.0,156.0

Step B. N,N-dimethyl-1H-pyrazol-3-amine: To a solution of tert-butyl 4-(dimethylamino)-1H-pyrazole-1-carboxylate (250 mg, 1 equiv) in DCM (0.5 mL) was added TFA (767 mg, 5.7 equiv). The reaction was stirred at 25 °C for 0.5 hours. The mixture was concentrated. The residue was basified by NaHCO₃ (2 mL) and extracted with ethyl acetate (2×10 mL). The combined organic layers were washed with brine (2×2 mL), dried over anhydrous sodium sulfate and concentrated to afford the title compound (130 mg, 99% yield) as colorless oil;

Step C. (3-(dimethylamino)-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (150 mg, 1.0 equiv) and N,N-dimethyl-1H-pyrazol-4-amine (53.6 mg, 2.0 equiv) in DMF (1 mL) were added HATU (183 mg, 2.0 equiv) and DIEA (93.5 mg, 3.0 equiv). The reaction was stirred at 25 °C for 0.5 hours. The mixture was purified by reversed phase flash chromatography [C18, water/ACN] to afford the title compound (10.3 mg, 5% yield) as white solid; ¹H NMR (400 MHz, CHLOROFORM-d) δ=8.94 (d, J=7.2 Hz, 1H), 7.53-7.46 (m, 3H), 7.19-7.08 (m, 2H), 7.06-6.94 (m, 1H), 5.39-5.19 (m, 1H), 4.60 (br dd, J=4.4, 8.4 Hz, 2H), 4.34-4.23 (m, 2H), 4.05-3.95 (m, 1H), 3.54-3.08 (m, 6H), 3.05-2.95 (m, 1H), 2.80 (s, 6H), 2.51-2.03 (m, 9H), 2.00-1.88 (m, 3H), 0.86-0.78 (m, 3H); LCMS (ESI, M+1): m/z=715.3.

### Example 65

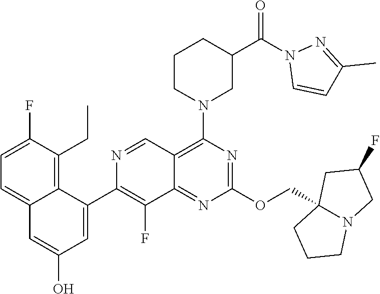 [SMILES: CCc1c(F)ccc2cc(O)cc(-c3ncc4c(N5CCCC(C(=O)n6ccc(C)n6)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)(3-methyl-1H-pyrazol-1-yl)methanone

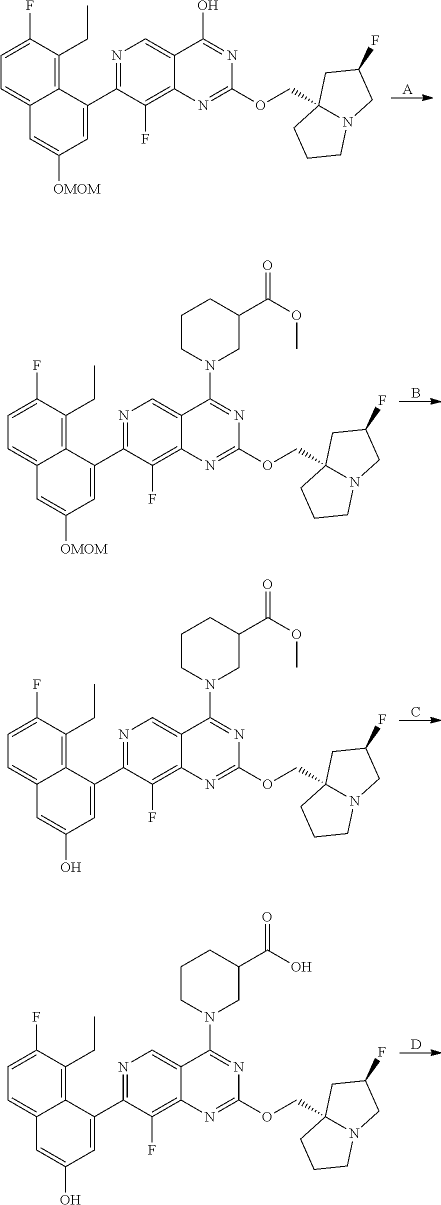

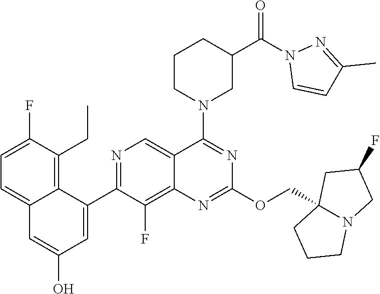 [SMILES: CCc1c(F)ccc2cc(O)cc(-c3ncc4c(N5CCCC(C(=O)n6ccc(C)n6)C5)nc(OC[C@@]56CCCN5C[C@H](F)C6)nc4c3F)c12]

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-3-carboxylate: To a solution 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (300 mg, 1.0 equiv), TEA (164 mg, 3.0 equiv) and benzotriazol-1-yloxyl-tris-(pyrrolidino)-phosphonium hexafluorophosphate (422 mg, 1.5 equiv) in DMSO (3 mL) was added methyl piperidine-3-carboxylate (155 mg, 2.0 equiv). The reaction was stirred at 30 °C for 6 hours. The mixture was diluted with water (10 mL) and extracted with ethyl acetate (3×10 mL). The combined organic layers were washed with brine (20 mL), dried over anhydrous sodium sulfate, concentrated and purified by reversed phase flash chromatography [C18, 0.1% formic acid condition] to afford the title compound (300 mg, 81% yield) as yellow oil; LCMS (ESI, M+1): m/z=680.4.

Step B. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-3-carboxylate: To a solution of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-3-carboxylate (150 mg, 1.0 equiv) in MeCN (0.5 mL) was added HCl·dioxane (4 M, 1.0 mL). The reaction was stirred at 0 °C for 0.5 hours. The mixture was adjusted to pH>8 with saturated NaHCO₃ aqueous (5 mL) at 0 °C and extracted with DCM (3×5 mL). The combined organic layers were washed with brine (10 mL), dried over anhydrous sodium sulfate, concentrated to afford the title compound (120 mg, crude) as yellow solid; LCMS (ESI, M+1): m/z=636.3.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-3-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-3-carboxylate (70.0 mg, 1.0 equiv) in THF (0.2 mL) and MeOH (0.3 mL) was added LiOH·H₂O (13.9 mg, 3.0 equiv) in H₂O (0.2 mL). The reaction was stirred at 20 °C for 1 hour. The mixture was adjusted to pH≈6 with HCl (4 M, 0.8 mL) at 0 °C and purified by reversed phase flash chromatography [C18, 0.1% formic acid condition] to afford the title compound (50.0 mg, 72% yield) as white solid; LCMS (ESI, M+1): m/z=622.3.

Step D. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-3-yl)(3-methyl-1H-pyrazol-1-yl)methanone: To a mixture of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-3-carboxylic acid (80.0 mg, 1.0 equiv), EDCI (37.0 mg, 1.5 equiv), HOBt (26.1 mg, 1.5 equiv) and DIEA (49.9 mg, 3.0 equiv) in DMF (1.0 mL) was added 3-Methylpyrazole (21.1 mg, 2.0 equiv). The reaction was stirred at 20 °C for 12 hours.

The mixture was filtered and purified by reversed phase flash chromatography [C18, neutral condition] to afford the title compound (18.5 mg, 20% yield) as yellow solid; ¹H NMR (400 MHz, DMSO-d₆) δ=9.94 (br d, J=5.2 Hz, 1H), 9.30 (d, J=5.6 Hz, 1H), 8.31-8.30 (m, 1H), 7.79-7.50 (m, 1H), 7.45-7.26 (m, 2H), 7.07-6.98 (m, 1H), 6.48-6.46 (m, 1H), 5.40-5.14 (m, 1H), 4.73-4.68 (m, 1H), 4.45-4.42 (m, 1H), 4.17-4.09 (m, 1H), 4.07-4.03 (m, 1H), 3.76-3.65 (m, 1H), 3.56-3.43 (m, 2H), 3.12-3.04 (m, 2H), 3.00 (br s, 1H), 2.85-2.78 (m, 1H), 2.33 (br s, 1H), 2.27 (d, J=10.4 Hz, 3H), 2.21-2.10 (m, 3H), 2.05 (br s, 2H), 1.95 (br d, J=8.8 Hz, 2H), 1.88-1.72 (m, 4H), 0.75-0.67 (m, 3H); LCMS (ESI, M+1): m/z=686.4.

### Example 66

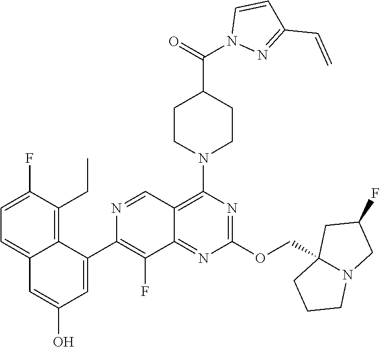 [SMILES: C=Cc1ccn(C(=O)C2CCN(c3nc(OC[C@@]45CCCN4C[C@H](F)C5)nc4c(F)c(-c5cc(O)cc6ccc(F)c(CC)c56)ncc34)CC2)n1]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-vinyl-1H-pyrazol -1-yl)methanone

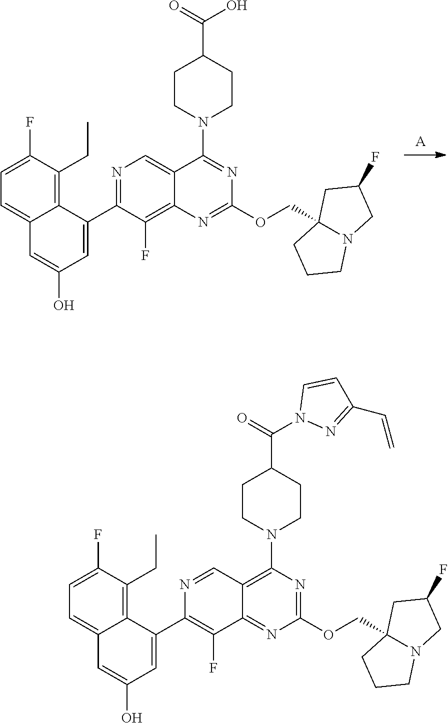

### Step A. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-vinyl-1H-pyrazol-1-yl)methanone

To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (300 mg, 1.0 equiv) and 3-vinyl-1H-pyrazole (300 mg, 6.6 equiv) in DMF (3 mL) were added HATU (367 mg, 2.0 equiv) and DIEA (312 mg, 5.0 equiv). The mixture was stirred at 25 °C for 0.5 hours. The mixture was filtered and purified by prep-HPLC [Waters Xbridge Prep OBD C18 150×40 mm×10 μm; A: water (NH₄HCO₃), B: ACN, B %: 45%-75% over 20 min] to afford the title compound (4.94 mg, 1.4% yield) as orange solid; ¹H NMR (400 MHz, CHLOROFORM-d) δ=8.98 (s, 1H), 8.20 (d, J=3.0 Hz, 1H), 7.63-7.51 (m, 1H), 7.23-7.13 (m, 2H), 7.03 (dd, J=2.4, 18.6 Hz, 1H), 6.77 (dd, J=11.2, 17.8 Hz, 1H), 6.63 (d, J=2.8 Hz, 1H), 5.91 (d, J=17.8 Hz, 1H), 5.58 (d, J=11.3 Hz, 1H), 5.41-5.19 (m, 1H), 4.70-4.58 (m, 2H), 4.36-4.21 (m, 2H), 4.09-3.96 (m, 1H), 3.61-3.42 (m, 2H), 3.37-3.12 (m, 3H), 3.05-2.94 (m, 1H), 2.57-2.40 (m, 1H), 2.31 (br s, 1H), 2.28-2.06 (m, 7H), 2.02-1.92 (m, 3H), 0.83 (br t, J=7.4 Hz, 3H); LCMS (ESI, M+1): m/z=698.4.

### Example 67

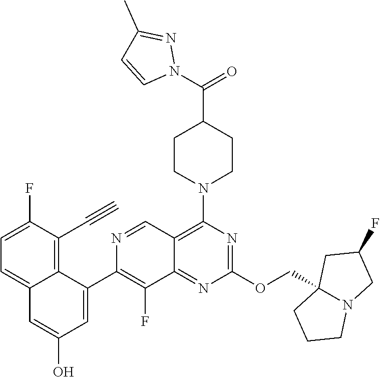 [SMILES: C#Cc1c(F)ccc2cc(O)cc(-c3ncc4c(N5CCC(C(=O)n6ccc(C)n6)CC5)nc(OC[C@@]56CCCN5C[C@H](F)C6)nc4c3F)c12]

### (1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-methyl-1H-pyrazol-1-yl)methanone

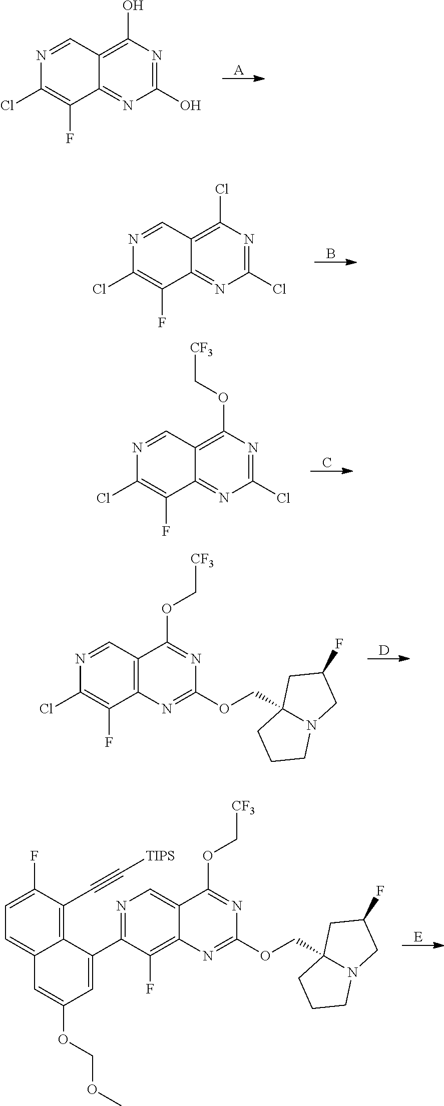

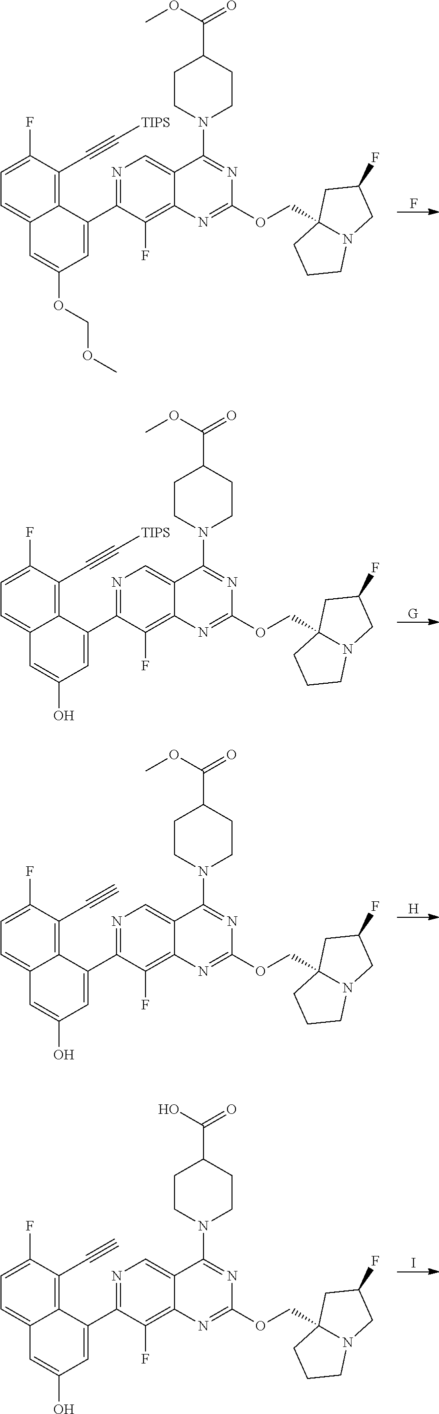

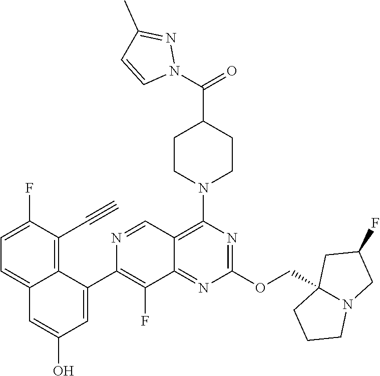 [SMILES: C#Cc1c(F)ccc2cc(O)cc(-c3ncc4c(N5CCC(C(=O)n6ccc(C)n6)CC5)nc(OC[C@@]56CCCN5C[C@H](F)C6)nc4c3F)c12]

Step A. 2,4,7-trichloro-8-fluoropyrido[4,3-d]pyrimidine; To a solution of POCl₃ (187 g, 5.0 equiv) in toluene (150 mL) were added 7-chloro-8-fluoropyrido[4,3-d]pyrimidine-2,4-diol (52.6 g, 1.0 equiv) and DIEA (69.4 g, 2.2 equiv). The reaction was stirred at 110 °C for 27 hours. The solvent was concentrated under vacuum. The mixture was poured into ice saturated NaHCO₃ solution (2.5 L) and maintained the pH was 8 then filtered. The filter cake was dissolved in ethyl acetate (1.5 L) and washed with saturated NaHCO₃ aqueous solution (1 L), saturated brine (1 L). The organic phase was dried over anhydrous sodium sulfate, concentrated under vacuum to afford the title compound (50 g, 81% yield) as brown solid.

Step B. 2,7-dichloro-8-fluoro-4-(2,2,2-trifluoroethoxy)pyrido[4,3-d]pyrimidine: To a solution of 2,2,2-trifluoroethanol (15.0 g, 0.9 equiv) in THF (150 mL) was added NaH (7.98 g, 60% purity, 1.2 equiv) at 0 °C. The mixture was stirred at 0 °C for 0.5 hour. Then the mixture was added to a solution of 2,4,7-trichloro-8-fluoropyrido[4,3-d]pyrimidine (42.0 g, 1.0 equiv) in THF (840 mL) at −40 °C. The reaction was stirred at −40 °C for 1 hour. The mixture was quenched with H₂O (500 mL) and extracted with ethyl acetate (3×300 mL). The combined organic layers were washed with brine (400 mL), dried over anhydrous sodium sulfate, concentrated and purified by column chromatography [SiO₂, petroleum ether/ethyl acetate 10/1 to 5/1] to afford the title compound (34.4 g, 53% yield) as yellow solid; LCMS [ESI, M+1]: 315.9.

Step C. 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: To a mixture of 2,7-dichloro-8-fluoro-4-(2,2,2-trifluoroethoxy)pyrido[4,3-d]pyrimidine (33.3 g, 1.0 equiv), DIEA (54.5 g, 4.0 equiv) and 4 Å molecular sieve (4.0 g) in THF (340 mL) was added ((2R,7aS)-2-fluorohexahydro-1H-pyrrolizin-7a-yl)methanol (20.1 g, 1.2 equiv). The reaction was stirred at 40 °C for 14 hours. The reaction mixture was diluted with H₂O (20 mL) and extracted with EtOAc (3×20 mL), the combined organic layers were dried over Na₂SO₄, filtered and concentrated under vacuum and purified by reversed phase flash chromatography [water (FA, 0.1%)/acetonitrile] to afford the title compound (28.8 g, 62% yield) as yellow solid; ¹H NMR (400 MHz, chloroform-d) δ 8.98 (s, 1H), 5.40-5.19 (m, 1H), 5.02 (q, J=8.0 Hz, 1H, 2H), 4.40-4.27 (m, 2H), 3.34-3.12 (m, 3H), 3.05-2.94 (m, 1H), 2.32-2.06 (m, 3H), 2.03-1.84 (m, 3H); LCMS [ESI, M+1]: 439.1.

Step D. 8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (2.20 g, 1.0 equiv) and 2-[2-fluoro-6-(methoxymethoxy)-8-(4,4,5,5-tetramethyl-1,3,2-dioxaborolan-2-yl)-1-naphthyl]ethynyl-triisopropyl-silane (3.85 g, 1.5 equiv) in methoxycyclopentane (25 mL) were added CataCxium (R) A Pd G3 (365 mg, 0.1 equiv) and Cs₂CO₃ (1.5 M in water, 2.5 equiv). The reaction was stirred at 100 °C for 3 hours under N₂ atmosphere. The mixture was diluted with water (30 mL) and extracted with ethyl acetate (40 mL). The organic layer was dried over sodium sulfate, concentrated and purified by reversed phase flash chromatography [C18, 0.1% formic acid condition] to afford the title compound (2.80 g, 64% yield) as a brown solid. LCMS (ESI, M+1): m/z=789.4.

Step E methyl 1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperidine-4-carboxylate: To a solution of 8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (1.20 g, 1.0 equiv) and methyl piperidine-4-carboxylate (653 mg, 3.0 equiv) in DMF (12 mL) were added DIEA (590 mg, 3.0 equiv) and 4 Å molecular sieve (1.00 g) at 25 °C. The reaction was stirred at 70 °C for 12 hours. The mixture was diluted with water (20 mL) and extracted with ethyl acetate (20 mL). The organic layer was dried over sodium sulfate, concentrated and purified by reversed phase flash chromatography [C18, 0.1% formic acid condition] to afford the title compound (800 mg, 56% yield) as a yellow solid. LCMS (ESI, M+1): m/z=832.5.

Step F. methyl 1-(8-fluoro-7-(7-fluoro-3-hydroxy-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperidine-4-carboxylate: To a solution of methyl 1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperidine-4-carboxylate (1.00 g, 1.0 equiv) in MeOH (10 mL) was added HCl/MeOH (2 M, 20 mL, 33.28 equiv) at 0 °C. The reaction was stirred at 0 °C for 0.5 hours. The mixture was concentrated under vacuum. The mixture was basified with NaHCO₃ (10 mL) and extracted with ethyl acetate (10 mL). The combined organic layers were dried over sodium sulfate and concentrated to afford the title compound (900 mg, crude) as yellow solid. LCMS (ESI, M+1): m/z=788.4.

Step G. methyl 1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate: To a solution of methyl 1-(8-fluoro-7-(7-fluoro-3-hydroxy-8-((triisopropylsilyl)ethynyl)naphthalen-1-yl)-2-(((2R,7aS)-2-fluorotetrahydro-1H-pyrrolizin-7a(5H)-yl)methoxy)pyrido[4,3-d]pyrimidin-4-yl)piperidine-4-carboxylate (900 mg, 1.0 equiv) in DMF (9 mL) was added CsF (2.26 g, 13 equiv). The reaction was stirred at 40 °C for 14 hours. The mixture was diluted with water (10 mL) and extracted with ethyl acetate (10 mL). The organic layer was dried over sodium sulfate and concentrated to afford the title compound (900 mg, crude) as yellow solid. LCMS (ESI, M+1): m/z=632.3.

Step H. 1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid: To a solution of methyl 1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate (500 mg, 1.0 equiv) in MeOH (5 mL) was added LiOH·H₂O (1.5 M in water, 2.11 mL, 4.0 equiv). The reaction was stirred at 25 °C for 0.5 hours. The mixture was concentrated, diluted with water (5 mL) and extracted with ethyl acetate (7 mL). The organic layer was dried over sodium sulfate, concentrated and purified by reversed phase flash chromatography [C18, 0.1% formic acid condition] to afford the title compound (300 mg, 58% yield) as a yellow solid. LCMS (ESI, M+1): m/z=618.2.

Step I. (1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-methyl-1H-pyrazol-1-yl)methanone: To a solution of 1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (150 mg, 242.87 μmol, 1 eq) and 3-methyl-1H-pyrazole (39.9 mg, 2.0 equiv) in DCM (0.5 mL) were added EDCI (69.8 mg, 1.5 equiv) and DMAP (44.5 mg, 1.5 equiv). The reaction was stirred at 25 °C for 12 hours. The mixture was concentrated, diluted with water (5 mL) and extracted with ethyl acetate (7 mL). The combined organic layers were dried over sodium sulfate, concentrated and purified by prep HPLC [Waters Xbridge Prep OBD C18 150×40 mm×10 μm; A: water (10 mM NH₄HCO₃), B: ACN, B %: 38%-68% over 20 min] to afford the title compound (31.5 mg, 18% yield) as orange solid. ¹H NMR (400 MHz, CHLOROFORM-d) δ=8.94 (d, J=4.4 Hz, 1H), 8.15 (d, J=2.8 Hz, 1H), 7.68-7.56 (m, 1H), 7.24-7.09 (m, 3H), 6.29 (d, J=2.8 Hz, 1H), 5.40-5.13 (m, 1H), 4.64 (br d, J=13.2 Hz, 2H), 4.31-4.19 (m, 2H), 4.07-3.97 (m, 1H), 3.52 (br t, J=11.2 Hz, 2H), 3.33-3.10 (m, 3H), 3.04-2.90 (m, 1H), 2.80 (s, 1H), 2.35 (s, 3H), 2.33-2.27 (m, 1H), 2.21 (br d, J=10.0 Hz, 3H), 2.11 (br d, J=12.8 Hz, 3H), 2.03-1.90 (m, 3H); LCMS (ESI, M+1): m/z=682.4.

### Example 68

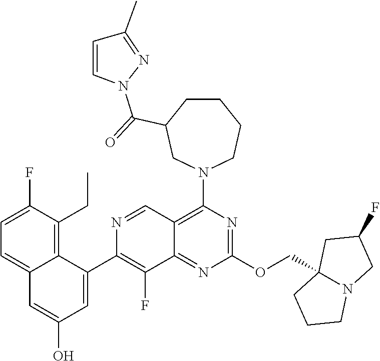 [SMILES: CCc1c(F)ccc2cc(O)cc(-c3ncc4c(N5CCCCC(C(=O)n6ccc(C)n6)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepan-3-yl)(3-methyl-1H-pyrazol-1-yl)methanone

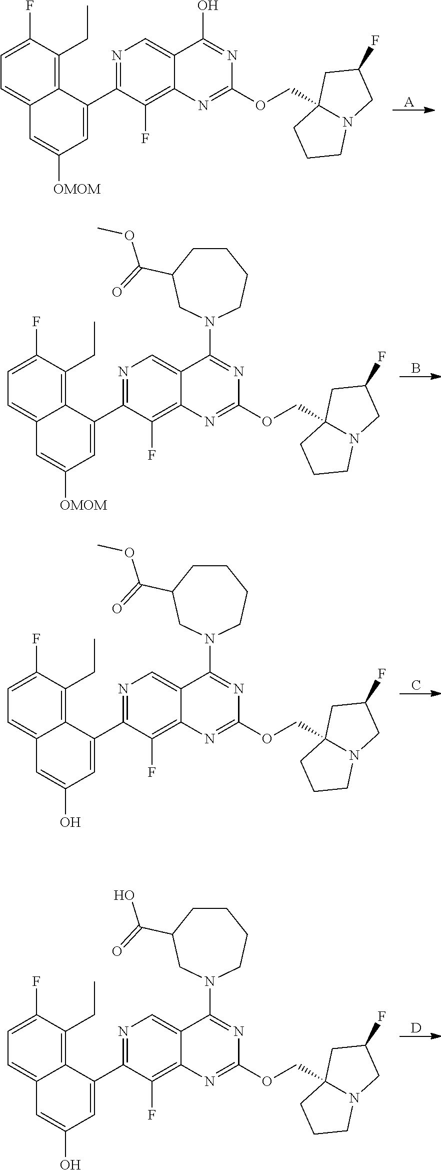

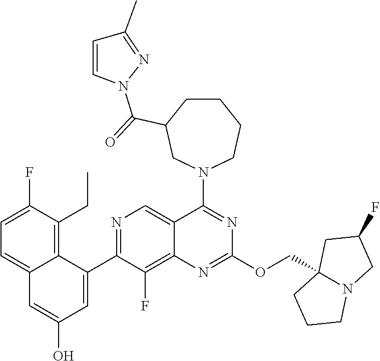 [SMILES: CCc1c(F)ccc2cc(O)cc(-c3ncc4c(N5CCCCC(C(=O)n6ccc(C)n6)C5)nc(OC[C@@]56CCCN5C[C@H](F)C6)nc4c3F)c12]

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-3-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 equiv) in DMSO (5 mL) was added TEA (456 mg, 5.0 equiv) and PyBOP (938 mg, 2.0 equiv). The reaction was stirred at 30 °C for 0.5 hours. Then methyl azepane-3-carboxylate (262 mg, 1.5 equiv, HCl) was added to the mixture. The reaction was stirred at 30 °C for 12 hours. The mixture was filtered and washed with DMSO (1 mL), and purified by reversed phase flash [C18, 0.1% formic acid condition] to afford the title compound (265 mg, 41% yield) as yellow solid; LCMS (ESI, M+1): m/z=694.3.

Step B. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-3-carboxylate: To a solution of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-3-carboxylate (143 mg, 1.0 equiv) in ACN (1 mL) was added HCl·dioxane (4 M, 2 mL, 39 equiv). The reaction was stirred at 20 °C for 0.5 hours. The mixture was adjusted pH to 8 with saturated NaHCO₃ aqueous solution (10 mL) and extracted with DCM (3×10 mL). The combined organic layers were dried over anhydrous sodium sulfate, concentrated to afford the title compound (160 mg, crude) as yellow solid; LCMS (ESI, M+1): m/z=650.4.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-3-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-3-carboxylate (160 mg, 1.0 equiv) in THF (0.6 mL), MeOH (0.4 mL) and H₂O (0.6 mL) was added LiOH·H₂O (31.0 mg, 3.0 equiv) at 0 °C The reaction was stirred at 20 °C for 2 hours. The mixture was filtered and purified by reversed phase flash [C18, 0.1% formic acid condition] to afford the title compound (100 mg, 63% yield) as yellow solid; LCMS (ESI, M+1): m/z=636.3.

Step D. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepan-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azepane-3-carboxylic acid (100 mg, 1.0 equiv), DIEA (61.0 mg, 3.0 equiv), HOBt (31.9 mg, 1.5 equiv) and EDCI (45.2 mg, 1.5 equiv) in DMF (0.5 mL) was added 3-methyl-1H-pyrazole (25.8 mg, 2.0 equiv). The reaction was stirred at 20 °C for 12 hours. The mixture was filtered and purified by reversed phase flash [C18, H₂O condition] to afford the title compound (17.6 mg, 15% yield) as white solid; ¹H NMR (400 MHz, DMSO-d₆) δ=9.93 (s, 1H), 9.26 (s, 1H), 8.34 (d, J=2.7 Hz, 1H), 7.76 (dd, J=6.0, 8.8 Hz, 1H), 7.43-7.28 (m, 2H), 7.01 (dd, J=2.4, 14.0 Hz, 1H), 6.50 (d, J=2.8 Hz, 1H), 5.30-5.04 (m, 1H), 4.63-4.48 (m, 1H), 4.26-4.06 (m, 3H), 4.01-3.70 (m, 3H), 3.05-2.87 (m, 3H), 2.75 (br s, 1H), 2.40-2.31 (m, 1H), 2.26 (s, 3H), 2.20-2.13 (m, 1H), 2.12-2.01 (m, 3H), 1.97-1.89 (m, 2H), 1.88-1.49 (m, 7H), 0.73 (td, J=7.6, 17.2 Hz, 3H); LCMS (ESI, M+1): m/z=700.3.

### Example 69

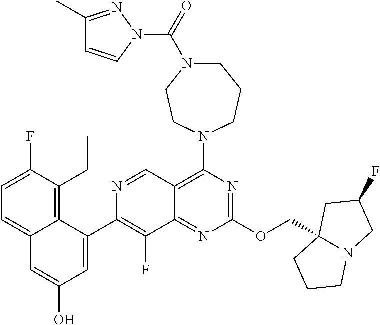 [SMILES: CCc1c(F)ccc2cc(O)cc(-c3ncc4c(N5CCCN(C(=O)n6ccc(C)n6)CC5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-diazepan-1-yl)(3-methyl-1H-pyrazol-1-yl)methanone

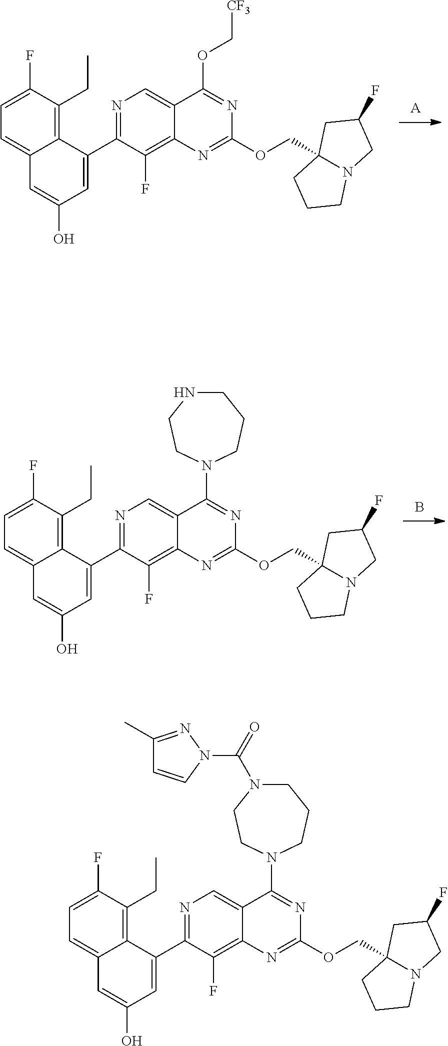

Step A. 4-(4-(1,4-diazepan-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a solution of 1,4-diazepane (169 mg, 2.5 equiv) in DMF (3.5 mL) was added 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (400 mg, 1.0 equiv). The reaction was stirred at 25 °C for 2 hours. The mixture was purified by reversed phase flash [water (0.1% formic acid)/acetonitrile=1/4] to afford the title compound (370 mg, 85.7% yield) as yellow solid; LCMS (ESI, M+1): m/z=593.3.

Step B. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-diazepan-1-yl)(3-methyl-1H-pyrazol-1-yl)methanone: To a solution of 5-methyl-1H-pyrazole (30.0 mg, 1.0 equiv), DIEA (189 mg, 4.0 equiv) in DCM (0.5 mL) was added bis(trichloromethyl) carbonate (320 mg, 2.9 equiv) at 0 °C. The reaction was stirred at 0 °C for 1 hour. 4-(4-(1,4-diazepan-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol (195 mg, 0.9 equiv) and DIEA (70.8 mg, 1.5 equiv) were added at 0 °C and the reaction was stirred at 20 °C for 14 hours. The mixture was quenched with saturated NaHCO₃ aqueous (10 mL) and extracted with DCM (4×10 mL). The combined organic layers were washed with brine (20 mL), dried over anhydrous sodium sulfate, concentrated and purified by reversed phase flash [water (0.1% formic acid)/acetonitrile=1/1] and prep-HPLC [column: Waters Xbridge 150×25 mm×5 μm; mobile phase: (water (ammonia hydroxide v/v)-acetonitrile); gradient: 46%-76% B over 10 minutes] to afford the title compound (23.1 mg, 9% yield) as white solid; ¹H NMR (400 MHz, DMSO-d6) δ=9.90 (br s, 1H), 9.18 (s, 1H), 7.98 (s, 1H), 7.76 (dd, J=6.0, 9.2 Hz, 1H), 7.39-7.29 (m, 2H), 7.02 (d, J=2.4 Hz, 1H), 6.25 (d, J=2.4 Hz, 1H), 5.37-5.16 (m, 1H), 4.41-4.26 (m, 2H), 4.25-4.00 (m, 6H), 3.97-3.69 (m, 2H), 3.14-3.02 (m, 2H), 3.00 (s, 1H), 2.86-2.76 (m, 1H), 2.39-2.26 (m, 1H), 2.22-1.93 (m, 9H), 1.89-1.67 (m, 3H), 0.76-0.64 (m, 3H); LCMS (ESI, M+1): m/z=701.5.

### Example 70

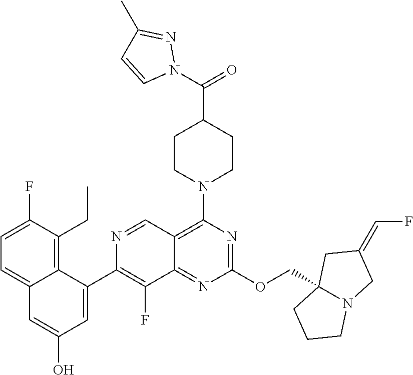 [SMILES: CCc1c(F)ccc2cc(O)cc(-c3ncc4c(N5CCC(C(=O)n6ccc(C)n6)CC5)nc(OC[C@@]56CCCN5C/C(=C\F)C6)nc4c3F)c12]

### (S,Z)-(1-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidin-4-yl)(3-methyl-1H-pyrazol-1-yl)methanone

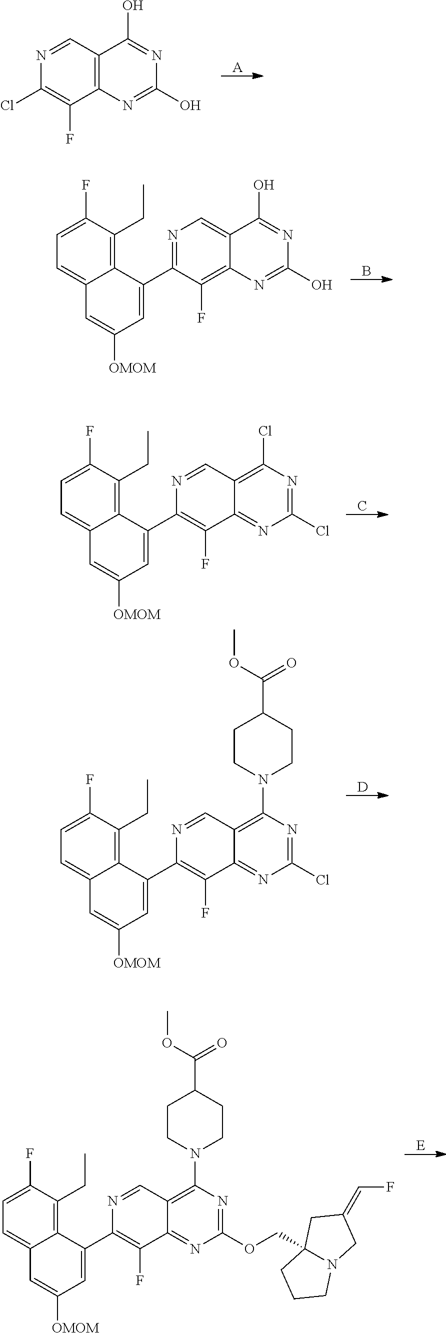

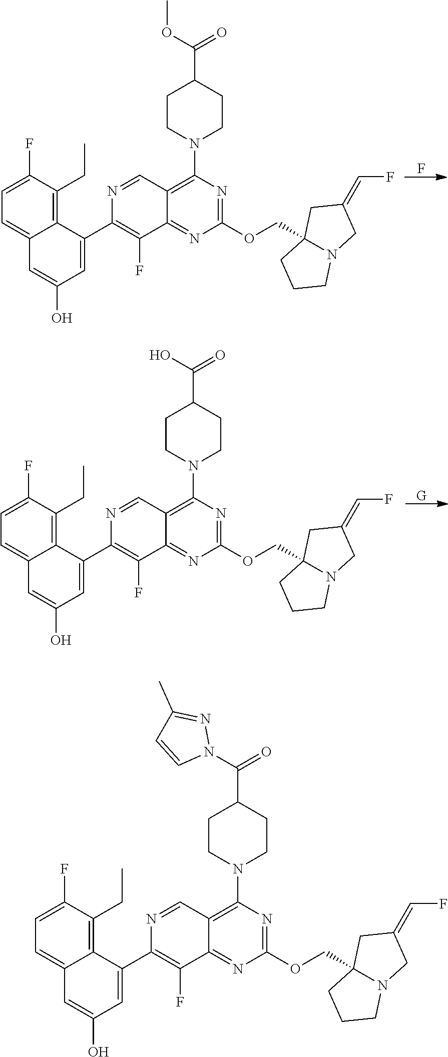

Step A. 7-(8-ethyl-7-fluoro-3-(methoxymethoxy)naphthalen-1-yl)-8-fluoropyrido[4,3-d]pyrimidine-2,4-diol: To a mixture of 7-chloro-8-fluoro-pyrido[4,3-d]pyrimidine-2,4-diol (150 g, 1.0 equiv) and 2-[8-ethyl-7-fluoro-3-(methoxymethoxy)-1-naphthyl]-4,4,5,5-tetramethyl-1,3,2-dioxaborolane (263 g, 1.1 equiv) in dimethyl formamide (1500 mL) and water (300 mL) were added cataCXium® A Pd G3 (25.3 g, 0.05 equiv) and potassium phosphate (295 g, 2.0 equiv). The reaction was degassed and purged with nitrogen for 3 times and stirred at 100 °C for 12 hours under nitrogen atmosphere. The mixture was filtered and the filter cake was washed with dimethyl formamide (300 mL). The filtrate was concentrated, triturated with water (2000 mL), filtered and washed with water (1000 mL) and ethanol (500 mL). The crude product was triturated with methyl tert-butyl ether (600 mL), filtered, and washed with methyl tert-butyl ether (200 mL) to afford 7-[8-ethyl-7-fluoro-3-(methoxymethoxy)-1-naphthyl]-8-fluoro-pyrido[4,3-d]pyrimidine-2,4-diol (102 g, 34.0% yield) as a yellow solid; ¹H NMR (400 MHz, DMSO-d6) δ=12.29-11.37 (m, 2H), 8.84 (s, 1H), 7.90 (dd, J=6.0, 8.8 Hz, 1H), 7.68 (d, J=2.8 Hz, 1H), 7.44 (t, J=9.2 Hz, 1H), 7.21 (d, J=2.4 Hz, 1H), 5.34 (s, 2H), 3.42 (s, 3H), 2.42-2.29 (m, 1H), 2.25-2.12 (m, 1H), 0.79 (t, J=7.2 Hz, 3H).

Step B. 2,4-dichloro-7-(8-ethyl-7-fluoro-3-(methoxymethoxy)naphthalen-1-yl)-8-fluoropyrido[4,3-d]pyrimidine; To a solution of 7-(8-ethyl-7-fluoro-3-(methoxymethoxy)naphthalen-1-yl)-8-fluoropyrido[4,3-d]pyrimidine-2,4-diol (1.80 g, 1.0 equiv) in toluene (30.0 mL) was added dropwise POCl₃ (3.34 g, 5.0 equiv) and DIEA (2.28 mL, 3.0 equiv) at 0 °C. The reaction was stirred at 100 °C for 1 hour. The mixture was concentrated then poured into saturated NaHCO₃ solution (100 mL) and extracted with ethyl acetate (2×50 mL). The combined organic layers were dried over sodium sulfate, concentrated and purified by column chromatography (SiO₂, petroleum ether/ethyl acetate 20/1 to 5/1) to afford the title compound (1.60 g, 81% yeild) as yellow oil; ¹H NMR (400 MHz, CHLOROFORM-d) δ=9.54 (s, 1H), 7.73 (dd, J=6.0, 9.2 Hz, 1H), 7.59 (d, J=2.8 Hz, 1H), 7.30 (t, J=9.2 Hz, 1H), 7.20 (d, J=2.8 Hz, 1H), 5.31 (d, J=2.0 Hz, 2H), 3.53 (s, 3H), 2.48-2.35 (m, 1H), 2.21-2.07 (m, 1H), 0.81 (t, J=7.4 Hz, 3H).

Step C. methyl 1-(2-chloro-7-(8-ethyl-7-fluoro-3-(methoxymethoxy)naphthalen-1-yl)-8-fluoropyrido[4,3-d]pyrimidin-4-yl)piperidine-4-carboxylate: To a solution of 2,4-dichloro-7-(8-ethyl-7-fluoro-3-(methoxymethoxy)naphthalen-1-yl)-8-fluoropyrido[4,3-d]pyrimidine (610 mg, 1.0 equiv) in DCM (12 mL) was added TEA (411 mg, 3 equiv) at −40 °C. A solution of methyl piperidine-4-carboxylate (155 mg, 0.8 equiv) in DCM (3 mL) was added dropwise to the mixture at −40 °C. The reaction was stirred at −40 °C for 1 hour. The mixture was diluted with water (20 mL) and extracted with dichloromethane (2×20 mL). The organic layers were dried over anhydrous sodium sulfate, concentrated and purified by silica gel chromatography (petroleum ether/ethyl acetate 20/1 to 2/1) to afford the title compound (940 mg, 56% yield) as yellow solid; ¹H NMR (400 MHz, METHANOL-d4) δ 9.18 (s, 1H), 7.84-7.76 (m, 1H), 7.63 (d, J=2.8 Hz, 1H), 7.32 (t, J=9.2 Hz, 1H), 7.23 (d, J=2.8 Hz, 1H), 5.33 (s, 2H), 4.68-4.59 (m, 2H), 3.73 (s, 3H), 3.72-3.63 (m, 2H), 3.50 (s, 3H), 2.93-2.83 (m, 1H), 2.54-2.40 (m, 1H), 2.27-2.13 (m, 3H), 2.02-1.89 (m, 2H), 0.81 (t, J=7.6 Hz, 3H).

Step D. methyl (S,Z)-1-(7-(8-ethyl-7-fluoro-3-(methoxymethoxy)naphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidine-4-carboxylate: To a solution of (S,Z)-(2-(fluoromethylene)tetrahydro-1H-pyrrolizin-7a(5H)-yl)methanol (169 mg, 1.1 equiv) in THF (10 mL) was added NaH (89.8 mg, 60% purity, 2.5 equiv) at 0 °C. The reaction was stirred at 0 °C for 10 minutes. Methyl 1-(2-chloro-7-(8-ethyl-7-fluoro-3-(methoxymethoxy)naphthalen-1-yl)-8-fluoropyrido[4,3-d]pyrimidin-4-yl)piperidine-4-carboxylate (500 mg, 1 equiv) was added into the mixture. The reaction was stirred at 0-20 °C for 1 hour. The mixture was quenched with water (5 mL) and extracted with ethyl acetate (3×10 mL). The combined organic layers were dried over anhydrous sodium sulfate, concentrated and purified by reversed phase flash chromatography [C18, 0.1% formic acid condition] to afford the title compound (300 mg, 43% yield) as yellow solid; LCMS (ESI, M+1): m/z=692.4.

Step E. methyl (S,Z)-1-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidine-4-carboxylate: To a mixture of methyl (S,Z)-1-(7-(8-ethyl-7-fluoro-3-(methoxymethoxy)naphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidine-4-carboxylate (350 mg, 1 equiv) in ACN (1.25 mL) was added HCl·dioxane (2 M, 5.06 mL, 20 equiv) dropwise. The reaction was stirred at 25 °C for 1 hour. The mixture was concentrated. The residue was basified with saturated NaHCO₃ solution (6 mL) and extracted with ethyl acetate (3×15 mL). The combined organic layers were dried over anhydrous sodium sulfate and concentrated to afford the title compound (300 mg, 82% yield) as yellow solid; LCMS (ESI, M+1): m/z=648.4.

Step F. (S,Z)-1-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidine-4-carboxylic acid: To a solution of methyl (S,Z)-1-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidine-4-carboxylate (300 mg, 1.0 equiv) in EtOH (4.5 mL) was added a solution of NaOH (92.6 mg, 5 equiv) in H₂O (1.5 mL). The reaction was stirred at 25 °C for 1 hour. The mixture was filtered and purified by reversed phase flash chromatography [water (NH₃·H₂O)/acetonitrile] to afford the title compound (200 mg, 67% yield) as yellow solid; LCMS (ESI, M+1): m/z=634.3.

Step G. (S,Z)-(1-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidin-4-yl)(3-methyl-1H-pyrazol-1-yl)methanone: To a solution of (S,Z)-1-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)piperidine-4-carboxylic acid (100 mg, 1 equiv) and EDCI (45.4 mg, 1.5 equiv) in DMF (1 mL) were added HOBt (25.6 mg, 1.2 equiv) and TEA (63.9 mg, 4.0 equiv) followed by 3-methyl-1H-pyrazole (38.9 mg, 3.0 equiv). The reaction was stirred at 25 °C for 12 hours. The mixture was filtered and purified by prep-HPLC [Phenomenex Luna C18 150×25 mm×10 μm; A: water (NH₄HCO₃), B: ACN, B %: 56%-86% over 10 min], followed by prep-HPLC [Phenomenex luna C18 150×25 mm×10 μm; A: water (FA), B: ACN, B %: 20%-50% over 1 min]. The desired fractions were diluted with water (60 mL) and extracted with dichloromethane (2×20 mL). The organic layers were dried over anhydrous sodium sulfate and concentrated to afford the title compound (2.50 mg, 2.2% yield) as white solid; ¹H NMR (400 MHz, CHLOROFORM-d3) δ 9.03-8.96 (m, 1H), 8.19-8.14 (m, 1H), 7.61-7.54 (m, 1H), 7.25-7.19 (m, 2H), 7.08-7.02 (m, 1H), 6.65-6.37 (m, 1H), 6.33-6.28 (m, 1H), 4.72-4.61 (m, 2H), 4.36-4.19 (m, 2H), 4.09-3.99 (m, 1H), 3.98-3.85 (m, 1H), 3.61-3.49 (m, 2H), 3.48-3.40 (m, 1H), 3.30-3.15 (m, 1H), 2.82-2.72 (m, 1H), 2.72-2.62 (m, 1H), 2.56-2.45 (m, 1H), 2.40-2.32 (m, 4H), 2.28-2.06 (m, 7H), 1.98-1.91 (m, 2H), 0.84 (t, J=7.2 Hz, 3H); LCMS (ESI, M+1): m/z=698.5.

### Example 71

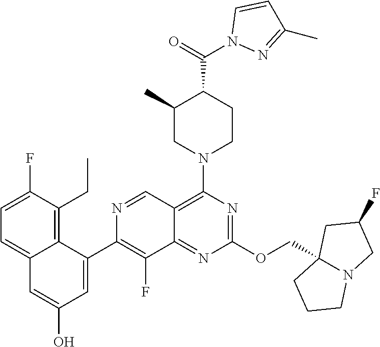 [SMILES: CCc1c(F)ccc2cc(O)cc(-c3ncc4c(N5CC[C@@H](C(=O)n6ccc(C)n6)[C@H](C)C5)nc(OC[C@@]56CCCN5C[C@H](F)C6)nc4c3F)c12]

### ((3S,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-4-yl)(3-methyl-1H-pyrazol-1-yl)methanone

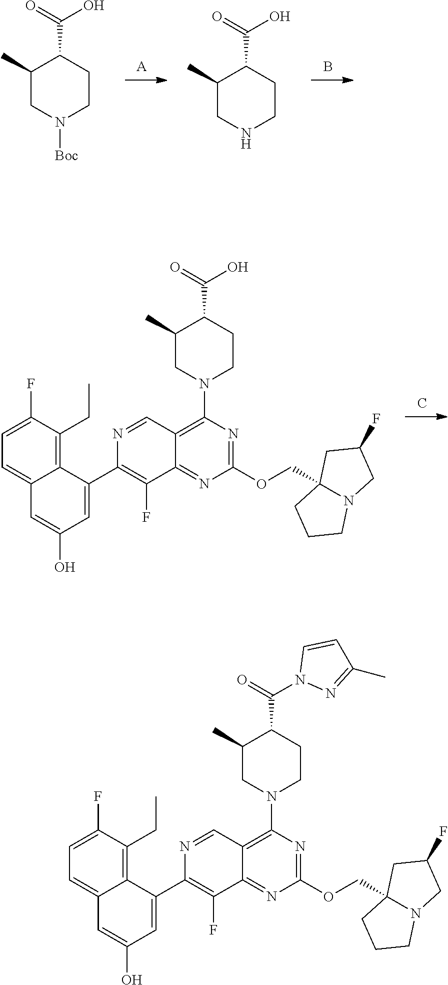

Step A. (3S,4R)-3-methylpiperidine-4-carboxylic acid: To a solution of (3S,4R)-1-(tert-butoxycarbonyl)-3-methylpiperidine-4-carboxylic acid (250 mg, 1.0 equiv) in ACN (0.5 mL) were added HCl·dioxane (2 M, 0.5 ml, 1.0 equiv). The reaction was stirred at 25 °C for 0.5 hours. The mixture was concentrated to afford the title compound (120 mg, 73% yield) as white solid; MS (ESI, M+1): m/z=144.4

Step B. (3S,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (130 mg, 1.0 equiv) in DMF (1.5 mL) were added (3S,4R)-3-methylpiperidine-4-carboxylic acid (62.8 mg, 2.0 equiv) and K₃PO₄ (279 mg, 6.0 equiv). The reaction was stirred at 60 °C for 0.5 hours. The mixture was purified by prep-HPLC [Phenomenex luna C18 150×25 mm×10 μm; A: water(FA)-ACN; B: CAN, B %: 15%-45% over 9 min] to afford the title compound (35 mg, 24% yield) as white solid; LCMS (ESI, M+1): m/z=636.4.

Step C. ((3S,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-4-yl)(3-methyl-1H-pyrazol-1-yl)methanone: To a solution of (3S,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylic acid (30 mg, 1.0 equiv) and 3-methyl-1H-pyrazole (11.6 mg, 3.0 equiv) in DMF (0.5 mL) were added HATU (35.9 mg, 2.0 equiv) and DIEA (18.3 mg, 3.0 equiv). The reaction was stirred at 25 °C for 0.5 hours. The mixture was purified by reversed phase flash chromatography [C18, water/ACN] to afford the title compound (18.0 mg, 54% yield) as white solid; ¹H NMR (400 MHz, CHLOROFORM-d) δ=8.97 (d, J=5.6 Hz, 1H), 8.20 (d, J=2.8 Hz, 1H), 7.62-7.52 (m, 1H), 7.25-7.17 (m, 2H), 7.05 (dd, J=2.8, 6.4 Hz, 1H), 6.33 (d, J=2.8 Hz, 1H), 5.40-5.21 (m, 1H), 4.77-4.69 (m, 1H), 4.64-4.55 (m, 1H), 4.29 (d, J=3.6 Hz, 2H), 3.84 (tt, J=3.6, 11.2 Hz, 1H), 3.43-3.34 (m, 1H), 3.33-3.16 (m, 3H), 3.15-3.04 (m, 1H), 3.04-2.96 (m, 1H), 2.55-2.36 (m, 5H), 2.36-2.29 (m, 1H), 2.27-2.18 (m, 3H), 2.17-2.11 (m, 1H), 2.01-1.90 (m, 4H), 1.01 (t, J=7.2 Hz, 3H), 0.88-0.80 (m, 3H); LCMS (ESI, M+1): m/z=700.4.

### Example 72

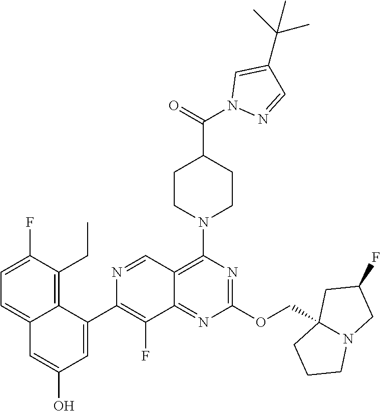 [SMILES: CCc1c(F)ccc2cc(O)cc(-c3ncc4c(N5CCC(C(=O)n6cc(C(C)(C)C)cn6)CC5)nc(OC[C@@]56CCCN5C[C@H](F)C6)nc4c3F)c12]

### (4-(tert-butyl)-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone

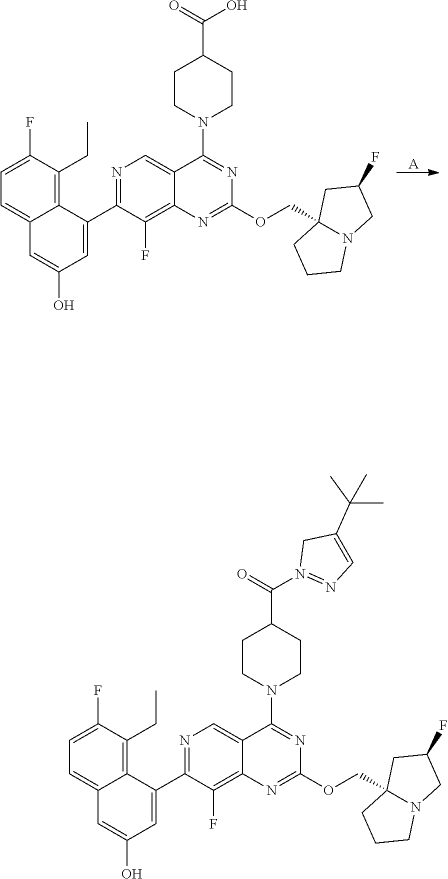

Step A. (4-(tert-butyl)-3H-2l4-pyrazol-2-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (50.0 mg, 1.0 equiv) and 4-tert-butyl-1H-pyrazole (20.0 mg, 2.0 equiv) in DMF (1 mL) was added EDCI (23.1 mg, 1.5 equiv), HOBt (5.43 mg, 0.5 equiv) and TEA (24.4 mg, 3.0 equiv). The reaction was stirred at 25 °C for 1 hour. The mixture was diluted with water (2 mL) and extracted with ethyl acetate (3 mL). The combined organic layer was dried over sodium sulfate, concentrated and purified by prep-HPLC [Waters Xbridge C18 150×25 mm×5 μm; A: water (10 mM NH₄HCO₃), B: ACN, B %: 62%-92% over 9 min] to afford the title compound (11.8 mg, 19% yield) as white solid. ¹H NMR (400 MHz, DMSO-d6) δ=9.93 (s, 1H), 9.10 (s, 1H), 8.16 (d, J=0.4 Hz, 1H), 7.97 (d, J=0.4 Hz, 1H), 7.76 (dd, J=6.0, 9.2 Hz, 1H), 7.37-7.30 (m, 2H), 7.02 (d, J=2.4 Hz, 1H), 5.41-5.14 (m, 1H), 4.63-4.52 (m, 2H), 4.20-4.11 (m, 1H), 4.09-3.95 (m, 2H), 3.67-3.50 (m, 2H), 3.16-2.98 (m, 4H), 2.87-2.77 (m, 1H), 2.14 (br d, J=10.0 Hz, 4H), 2.08-1.97 (m, 3H), 1.97-1.88 (m, 2H), 1.86-1.75 (m, 3H), 1.26 (s, 9H), 0.72 (t, J=7.2 Hz, 3H) LCMS (ESI, M+1): m/z=728.5.

### Example 73

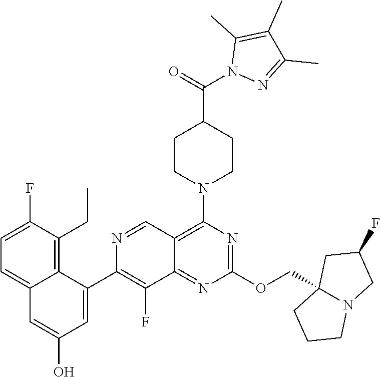 [SMILES: CCc1c(F)ccc2cc(O)cc(-c3ncc4c(N5CCC(C(=O)n6nc(C)c(C)c6C)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3,4,5-trimethyl-1H-pyrazol-1-yl)methanone

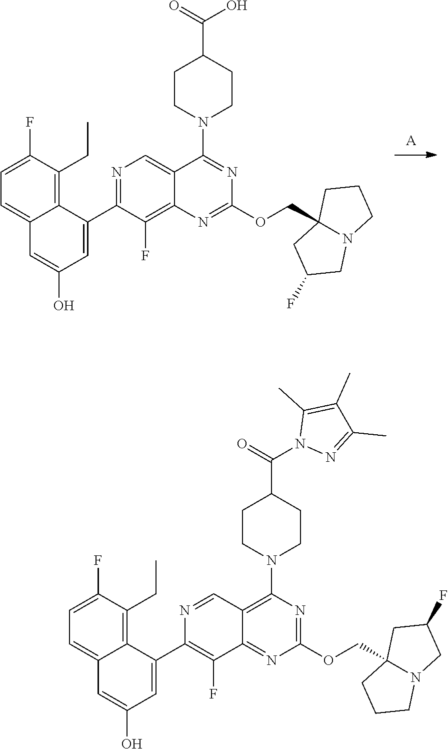

Step A. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3,4,5-trimethyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (50 mg, 1.0 equiv) and 3,4,5-trimethyl-1H-pyrazole (26.6 mg, 3.0 equiv) in DMF (1 mL) were added HATU (61.2 mg, 2.0 equiv) and DIEA (31.2 mg, 3.0 equiv). The reaction was stirred at 25 °C for 0.5 hours. The mixture was purified by prep-HPLC[Waters Xbridge 150×25 mm×5 μm; mobile phase: [water (NH4HCO3)-ACN]; gradient: 50%-80% B over 15 min] to afford the title compound (6.31 mg, 11% yield) as white solid; ¹H NMR (400 MHz, CHLOROFORM-d) δ=8.99 (s, 1H), 7.61-7.53 (m, 1H), 7.25-7.18 (m, 2H), 7.06 (dd, J=2.8, 16.8 Hz, 1H), 5.41-5.20 (m, 1H), 4.65 (br d, J=13.2 Hz, 2H), 4.34-4.21 (m, 2H), 4.15-4.05 (m, 1H), 3.60-3.44 (m, 2H), 3.33-3.13 (m, 3H), 3.05-2.95 (m, 1H), 2.49 (s, 4H), 2.31 (br d, J=6.0 Hz, 1H), 2.24 (s, 4H), 2.21-2.12 (m, 3H), 2.11-2.02 (m, 3H), 2.01-1.92 (m, 6H), 0.84 (t, J=7.6 Hz, 3H); LCMS (ESI, M+1): m/z=714.4.

### Example 74

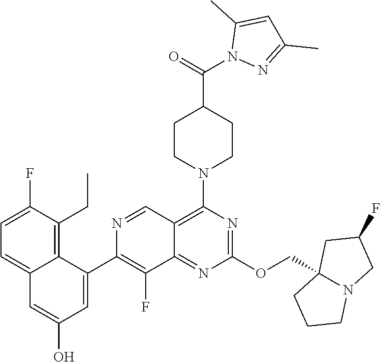 [SMILES: CCc1c(F)ccc2cc(O)cc(-c3ncc4c(N5CCC(C(=O)n6nc(C)cc6C)CC5)nc(OC[C@@]56CCCN5C[C@H](F)C6)nc4c3F)c12]

### (3,5-dimethyl-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone

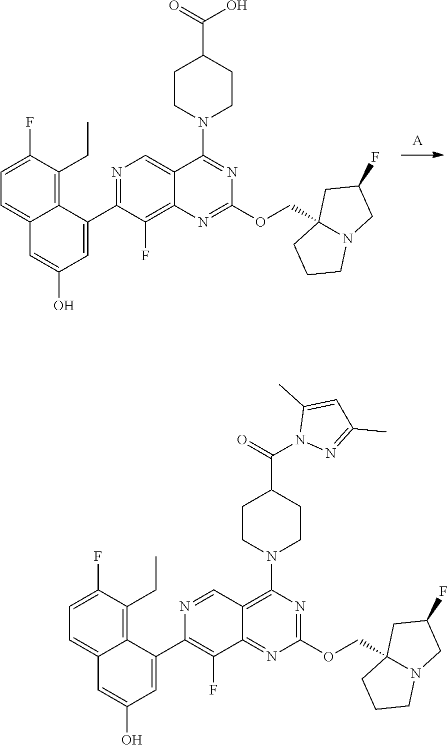

Step D. (3,5-dimethyl-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 equiv) and 3,5-dimethyl-1H-pyrazole (46.4 mg, 3.0 equiv) in DMF (1 mL) were added HATU (122 mg, 2.0 equiv) and DIEA (62.4 mg, 3.0 equiv). The reaction was stirred at 25 °C for 0.5 hours. The mixture was purified by reversed phase flash chromatography [C18,neutral condition] to afford the title compound (28.3 mg, 24% yield) as yellow solid; ¹H NMR (400 MHz, CHLOROFORM-d) δ=8.99 (s, 1H), 7.64-7.51 (m, 1H), 7.25-7.18 (m, 2H), 7.10-7.00 (m, 1H), 6.02 (s, 1H), 5.41-5.20 (m, 1H), 4.71-4.61 (m, 2H), 4.34-4.21 (m, 2H), 4.17-4.03 (m, 1H), 3.59-3.46 (m, 2H), 3.35-3.14 (m, 3H), 3.06-2.95 (m, 1H), 2.57 (s, 3H), 2.54-2.44 (m, 1H), 2.33-2.27 (m, 4H), 2.26-2.12 (m, 5H), 2.11-2.04 (m, 2H), 2.01-1.87 (m, 3H), 0.84 (t, J=7.6 Hz, 3H); LCMS (ESI, M+1): m/z=700.4.

### Example 75

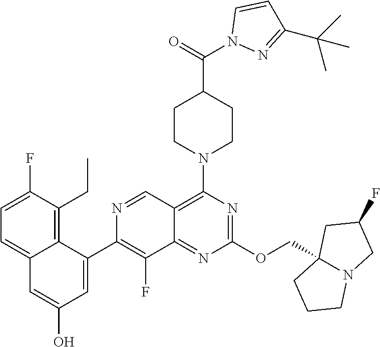 [SMILES: CCc1c(F)ccc2cc(O)cc(-c3ncc4c(N5CCC(C(=O)n6ccc(C(C)(C)C)n6)CC5)nc(OC[C@@]56CCCN5C[C@H](F)C6)nc4c3F)c12]

### (3-(tert-butyl)-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone

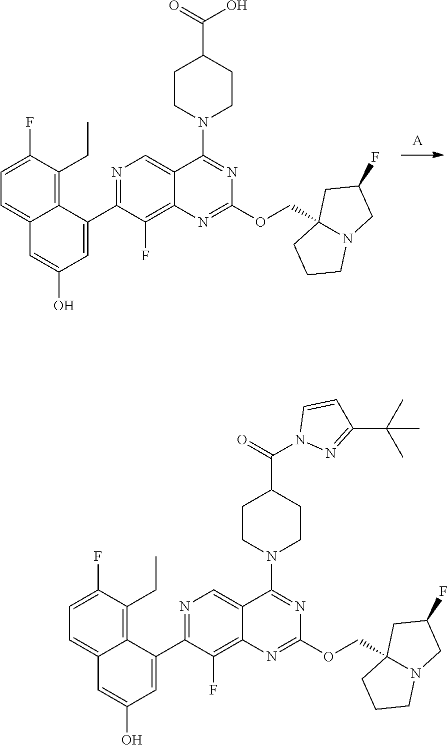

Step A. (3-(tert-butyl)-1H-pyrazol-1-yl)(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 equiv) and 3-tert-butyl-1H-pyrazole (59.9 mg, 3.0 equiv) in DMF (1 mL) were added HATU (91.7 mg, 1.5 equiv) and DIEA (62.4 mg, 3.0 equiv). The reaction was stirred at 25 °C for 1 hour. The mixture was filtered and purified by prep-HPLC [column: Waters Xbridge 150×25 mm×5 μm; A: water (NH₄HCO₃), B: ACN, B %: 54%-84% over 15 min]. The aqueous was extracted with ethyl acetate (2×10 mL). The combined organic layers were dried over anhydrous sodium sulfate and concentrated to afford the title compound (27.0 mg, 23% yield) as yellow solid; ¹H NMR (400 MHz, chloroform-d) δ=8.99 (s, 1H), 8.15 (d, J=2.8 Hz, 1H), 7.57 (dd, J=5.6, 9.2 Hz, 1H), 7.24-7.17 (m, 2H), 7.08-7.00 (m, 1H), 6.39 (d, J=2.8 Hz, 1H), 5.41-5.20 (m, 1H), 4.70-4.57 (m, 2H), 4.34-4.22 (m, 2H), 4.09-3.98 (m, 1H), 3.62-3.46 (m, 2H), 3.42-3.10 (m, 3H), 3.06-2.94 (m, 1H), 2.56-2.43 (m, 1H), 2.38-2.29 (m, 1H), 2.28-2.20 (m, 3H), 2.20-2.07 (m, 4H), 2.02-1.90 (m, 3H), 1.35 (s, 9H), 0.83 (t, J=7.2 Hz, 3H); LCMS (ESI, M+1): m/z=728.4.

### Example 76

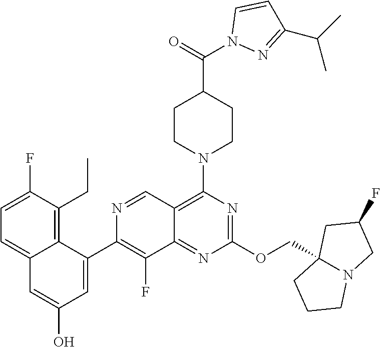 [SMILES: CCc1c(F)ccc2cc(O)cc(-c3ncc4c(N5CCC(C(=O)n6ccc(C(C)C)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-isopropyl-1H-pyrazol-1-yl)methanone

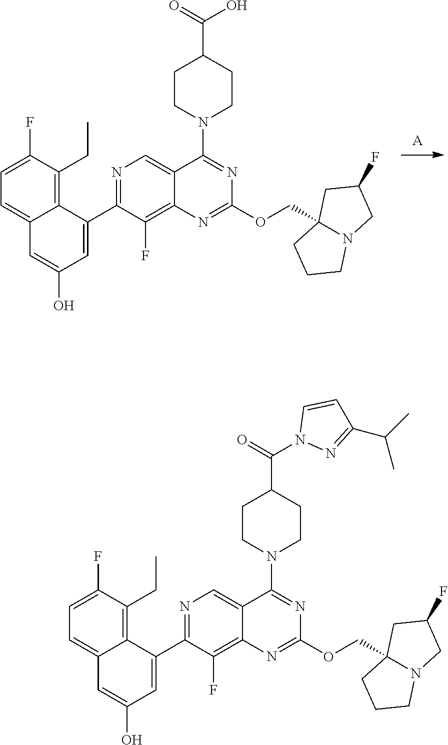

Step A. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(3-isopropyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 equiv) and 3-isopropyl-1H-pyrazole (53.2 mg, 3.0 equiv) in DMF (1 mL) were added HATU (91.7 mg, 1.5 equiv) and DIEA (62.4 mg, 3.0 equiv). The reaction was stirred at 25 °C for 2 hours. The mixture was filtered and purified by prep-HPLC [Waters Xbridge 150×25 mm×5 μm; A: water (NH₄HCO₃), B: ACN, B %: 50%-80% over 15 min] to afford the title compound (13.9 mg, 12% yield) as yellow solid; ¹H NMR (400 MHz, CHLOROFORM-d) δ=8.97 (s, 1H), 8.16 (d, J=2.8 Hz, 1H), 7.58-7.51 (m, 1H), 7.22-7.14 (m, 2H), 7.07-6.96 (m, 1H), 6.34 (d, J=2.8 Hz, 1H), 5.41-5.18 (m, 1H), 4.70-4.55 (m, 2H), 4.36-4.24 (m, 2H), 4.10-3.95 (m, 1H), 3.60-3.45 (m, 2H), 3.40-3.11 (m, 3H), 3.09-2.94 (m, 2H), 2.55-2.43 (m, 1H), 2.38-2.28 (m, 1H), 2.27-2.17 (m, 4H), 2.17-2.08 (m, 3H), 2.00-1.90 (m, 3H), 1.31 (d, J=6.8 Hz, 6H), 0.82 (t, J=7.2 Hz, 3H); LCMS (ESI, M+1): m/z=714.3.

### Example 77

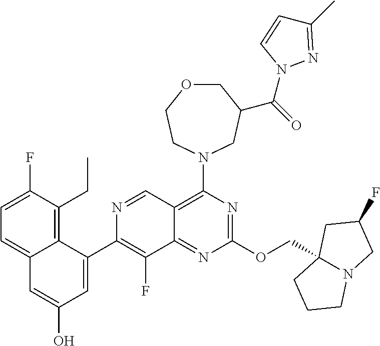 [SMILES: CCc1c(F)ccc2cc(O)cc(-c3ncc4c(N5CCOCC(C(=O)n6ccc(C)n6)C5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)(3-methyl-1H-pyrazol-1-yl)methanone

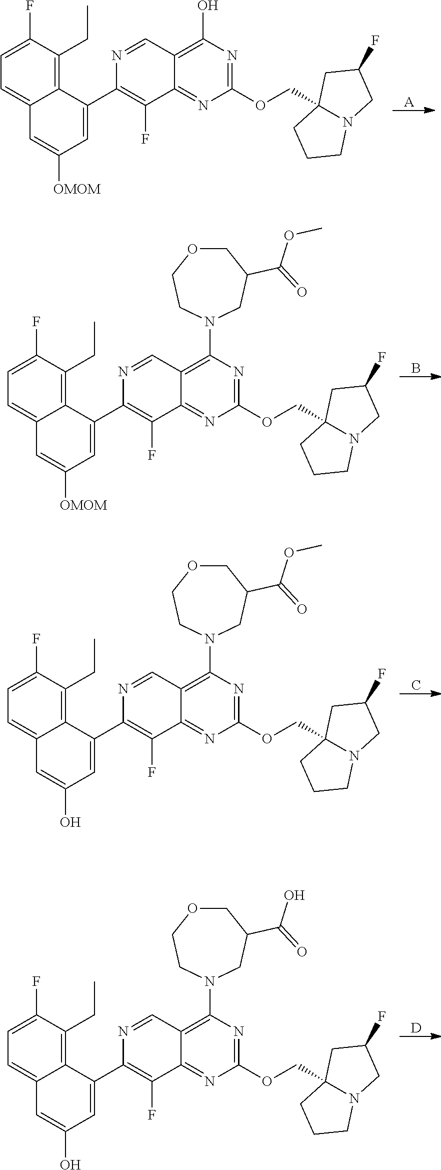

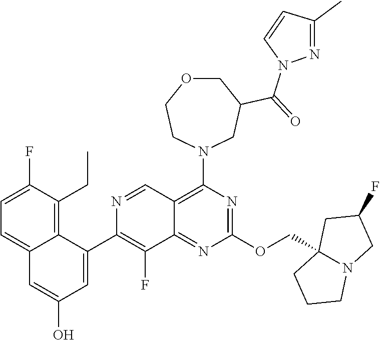 [SMILES: CCc1c(F)ccc2cc(O)cc(-c3ncc4c(N5CCOCC(C(=O)n6ccc(C)n6)C5)nc(OC[C@@]56CCCN5C[C@H](F)C6)nc4c3F)c12]

Step A. methyl 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (100 mg, 1.0 equiv) in DMF (2 mL) were added DIEA (70.0 mg, 3.0 equiv) and PyBOP (141 mg, 1.5 equiv) slowly at 25 °C, the reaction was stirred at 25 °C for 20 minutes. And then methyl 1,4-oxazepane-6-carboxylate (34.4 mg, 1.2 equiv) was added at 25 °C slowly. The reaction was stirred at 25 °C for 2 hours. The mixture was filtered, concentrated and purified by prep-HPLC [C18 150×25 mm×10 μm; A: water (ammonia hydroxide v/v), B: ACN, B %: 40%-70% over 10 min] to afford the title compound (96.0 mg, 77% yield) as white solid; LCMS (ESI, M+1): m/z=696.5.

Step B. methyl 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxylate: A solution of methyl 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxylate (50.0 mg, 1.0 equiv) in MeOH (0.9 mL) was added HCl·MeOH (2 M, 2 mL, 111 equiv) at 0 °C. The reaction was stirred at 25 °C for 1 hour. The mixture was concentrated, diluted with MeOH (3 mL), adjusted to pH about 7 with NaHCO₃solid, filtered and concentrated to afford the title compound (45.0 mg, 78% yield) as white solid; LCMS (ESI, M+1): m/z=652.3.

Step C. 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxylic acid: To a solution of methyl 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxylate (35.0 mg, 1.0 equiv) in MeOH (1 mL) was added LiOH·H₂O (2 M, 1 mL, 37 equiv). The reaction was stirred at 25 °C for 1 hour. The mixture was filtered and purified by reversed-phase flash (0.1% FA condition) to afford the title compound (18.0 mg, 48% yield, HCOOH) as white solid; LCMS (ESI, M+1): m/z=638.2.

Step D. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl)(3-methyl-1H-pyrazol-1-yl)methanone: To a solution of 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepane-6-carboxylic acid (16.0 mg, 1.0 equiv, HCOOH) and 3-methyl-1H-pyrazole (5.76 mg, 3.0 equiv) and DIEA (15.1 mg, 5.0 equiv) in DMF (0.5 mL) was added HATU (17.8 mg, 2.0 equiv). The reaction was stirred at 25 °C for 2 hours. The mixture was filtered and purified by prep-HPLC [Waters Xbridge 150×25 mm×5 μm; A: water (NH₄HCO₃), B: ACN, B %: 40%-70% over 9 min] to afford the title compound (4.65 mg, 26%) as yellow solid; ¹H NMR (400 MHz, CHLOROFORM-d) δ=8.83 (t, J=3.6 Hz, 1H), 8.11-7.98 (m, 1H), 7.67-7.52 (m, 1H), 7.23-7.09 (m, 2H), 6.84-6.62 (m, 1H), 6.29-6.17 (m, 1H), 5.43-5.20 (m, 2H), 4.51-4.31 (m, 1H), 4.28-4.17 (m, 1H), 4.04-3.85 (m, 2H), 3.83-3.54 (m, 4H), 3.43-3.14 (m, 3H), 3.09-2.98 (m, 1H), 2.53-2.38 (m, 2H), 2.36-2.31 (m, 2H), 2.30-2.23 (m, 3H), 2.22-2.08 (m, 5H), 2.02-1.99 (m, 2H), 0.82-0.72 (m, 3H); LCMS (ESI, M+1): m/z=702.2.

### Example 78

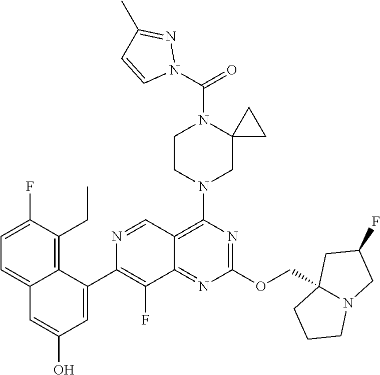 [SMILES: CCc1c(F)ccc2cc(O)cc(-c3ncc4c(N5CCN(C(=O)n6ccc(C)n6)C6(CC6)C5)nc(OC[C@@]56CCCN5C[C@H](F)C6)nc4c3F)c12]

### (7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,7-diazaspiro[2.5]octan-4-yl)(3-methyl-1H-pyrazol-1-yl)methanone

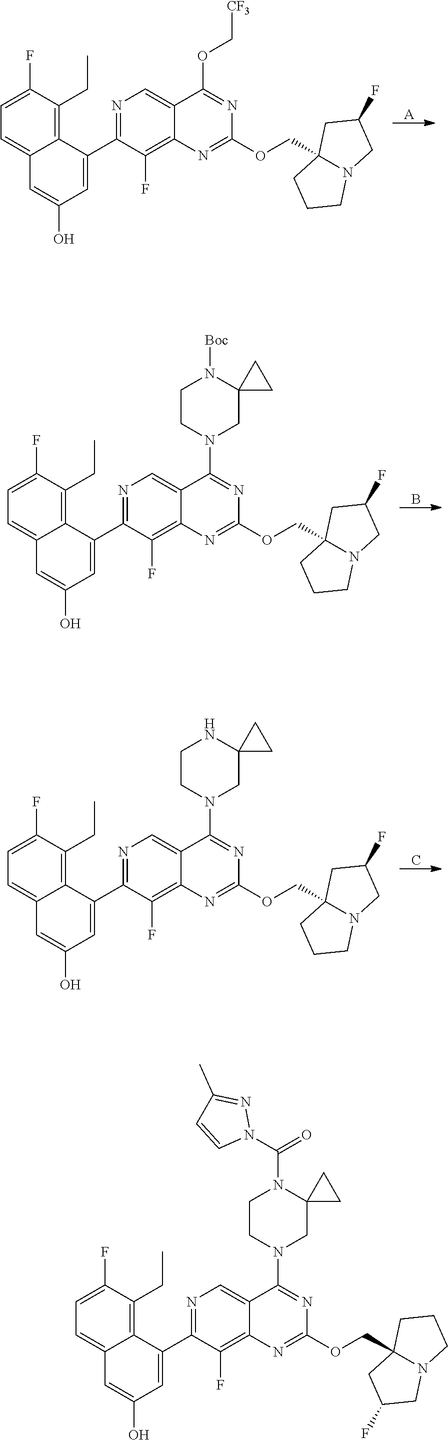

Step A. tert-butyl 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,7-diazaspiro[2.5]octane-4-carboxylate: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (500 mg, 1.0 equiv) in DMF (6 mL) were added tert-butyl 4,7-diazaspiro[2.5]octane-4-carboxylate (627 mg, 3.5 equiv) and DIEA (218 mg, 2.0 equiv). The reaction was stirred at 35 °C for 10 hours. The mixture was filtered and purified by reversed phase flash [C18, 0.1% formic acid condition] to afford the title compound (450 mg, 74% yield) as yellow solid; LCMS (ESI, M+1): m/z=705.4.

Step B. 5-ethyl-6-fluoro-4-(8-fluoro-2-(((2R,7aS)-2-fluorotetrahydro-1H-pyrrolizin-7a(5H)-yl)methoxy)-4-(4,7-diazaspiro[2.5]octan-7-yl)pyrido[4,3-d]pyrimidin-7-yl)naphthalen-2-ol: To a solution of tert-butyl 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,7-diazaspiro[2.5]octane-4-carboxylate (150 mg, 1.0 equiv) in MeOH (1 mL) was added HCl·MeOH (2 M, 3 mL) at 0 °C. The reaction was stirred at 0 °C for 1 hour. The mixture was adjusted to pH=7 with saturated NaHCO₃ (10 mL) and extracted with DCM (2×10 mL). The combined organic layers were washed with brine (30 mL), dried over anhydrous sodium sulfate, filtered and concentrated to afford the title compound (120 mg, crude) as white solid.

Step C. (7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,7-diazaspiro[2.5]octan-4-yl)(3-methyl-1H-pyrazol-1-yl)methanone: To a solution of 3-methyl-1H-pyrazole (20.0 mg, 1.0 equiv) and DIEA (126 mg, 4.0 equiv) in DCM (0.5 mL) was added bis(trichloromethyl) carbonate (120 mg, 1.7 equiv). The reaction was stirred at 0 °C for 1 hour. To the mixture were added 5-ethyl-6-fluoro-4-(8-fluoro-2-(((2R,7aS)-2-fluorotetrahydro-1H-pyrrolizin-7a(5H)-yl)methoxy)-4-(4,7-diazaspiro[2.5]octan-7-yl)pyrido[4,3-d]pyrimidin-7-yl)naphthalen-2-ol (118 mg, 0.8 equiv) and DIEA (47.2 mg, 1.5 equiv) in DCM (0.5 mL) at 0 °C. The reaction was stirred at 20 °C for 12 hours. The mixture was quenched with saturated NaHCO₃ aqueous (10 mL) and extracted with DCM (4×10 mL). The combined organic layers were washed with brine (20 mL), dried over anhydrous sodium sulfate, concentrated and purified prep-HPLC [column: Phenomenex luna C18 150×25 mm×10 μm; A: water(FA), B: ACN; B %: 20%-50% over 10 min] and SFC [column: DAICEL CHIRALCEL OX (250 mm×30 mm, 10 μm); A: CO₂-ACN, B: MeOH (0.1% NH₃·H₂O); B %: 40%, isocratic elution mode] to afford two peaks.

Peak 1:(7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,7-diazaspiro[2.5]octan-4-yl)(5-methyl-1H-pyrazol-1-yl)methanone: The residue was purified prep-HPLC [column: Phenomenex luna C18 150×25 mm×10 μm; A: water(FA), B: ACN; B %: 22%-52% over 10 min] to afford the title compound (6.63 mg, 3.8% yield) as off-white gum; ¹H NMR (400 MHz, DMSO-d6) δ=10.00 (s, 1H), 9.16 (s, 1H), 7.77 (dd, J=6.0, 9.2 Hz, 1H), 7.54 (s, 1H), 7.41-7.29 (m, 2H), 7.04 (d, J=2.4 Hz, 1H), 6.26 (s, 1H), 5.59-5.34 (m, 1H), 4.41-4.14 (m, 6H), 3.89 (br d, J=3.6 Hz, 2H), 3.46 (br d, J=6.8 Hz, 2H), 2.44-1.84 (m, 13H), 1.02-0.78 (m, 4H), 0.71 (t, J=7.2 Hz, 3H); LCMS (ESI, M+1): m/z=713.3; SFC: 96.3% de, Column: Chiralcel OX-3 50×4.6 mm I.D., 3 μm Mobile phase: Phase A for CO₂, and Phase B for MeOH+ACN(0.05% DEA); Gradient elution: 30% MeOH+ACN (0.05% DEA) in CO₂ Flow rate: 3 mL/min; Detector: PDA Column Temp: 35C; Back Pressure: 100Bar, t_{R}: 2.315 min.

Peak 2: (7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,7-diazaspiro[2.5]octan-4-yl)(3-methyl-1H-pyrazol-1-yl)methanone: The residue was purified prep-HPLC [column: Phenomenex luna C18 150×25 mm×10 μm; A: water(FA), B: ACN; B %: 22%-52% over 10 min] to afford the title compound (11.8 mg, 6.6% yield) as off-white gum; ¹H NMR (400 MHz, DMSO-d6) δ=10.03 (br s, 1H), 9.13 (s, 1H), 8.07 (d, J=2.4 Hz, 1H), 7.76 (dd, J=6.0, 8.8 Hz, 1H), 7.39-7.28 (m, 2H), 7.05 (s, 1H), 6.31 (d, J=2.4 Hz, 1H), 5.47-5.26 (m, 1H), 4.32-4.08 (m, 8H), 2.97 (br d, J=5.2 Hz, 2H), 2.33 (br s, 2H), 2.26-1.76 (m, 11H), 1.05-0.95 (m, 2H), 0.90 (br s, 2H), 0.71 (br t, J=7.2 Hz, 3H); LCMS (ESI, M+1): m/z=713.3; SFC: 97.3% de, Column: Chiralcel OX-3 50×4.6 mm I.D., 3 μm Mobile phase: Phase A for CO₂, and Phase B for MeOH+ACN (0.05% DEA); Gradient elution: 30% MeOH+ACN (0.05% DEA) in CO₂ Flow rate: 3 mL/min; Detector: PDA Column Temp: 35C; Back Pressure: 100Bar, t_{R}: 2.765 min.

### Example 79

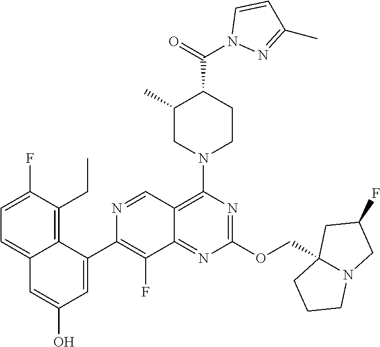 [SMILES: CCc1c(F)ccc2cc(O)cc(-c3ncc4c(N5CC[C@@H](C(=O)n6ccc(C)n6)[C@@H](C)C5)nc(OC[C@@]56CCCN5C[C@H](F)C6)nc4c3F)c12]

### ((3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-4-yl)(3-methyl-1H-pyrazol-1-yl)methanone

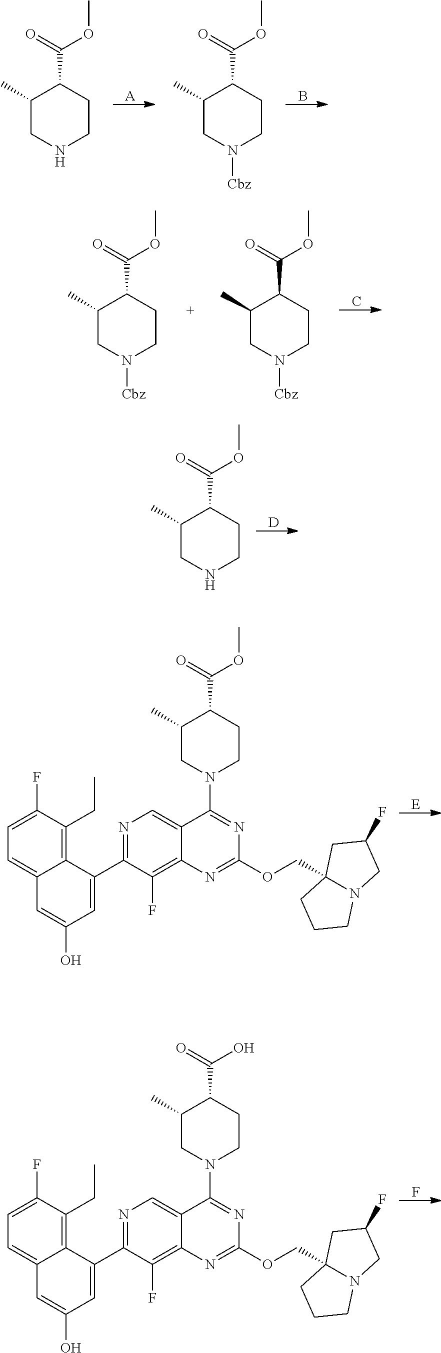

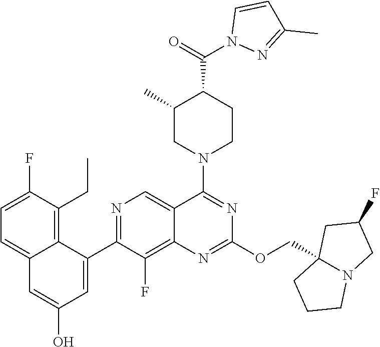 [SMILES: CCc1c(F)ccc2cc(O)cc(-c3ncc4c(N5CC[C@@H](C(=O)n6ccc(C)n6)[C@@H](C)C5)nc(OC[C@@]56CCCN5C[C@H](F)C6)nc4c3F)c12]

Step A. cis-1-benzyl 4-methyl (3R,4R)-3-methylpiperidine-1,4-dicarboxylate: To a solution of cis-methyl (3R,4R)-3-methylpiperidine-4-carboxylate (800 mg, 1.0 equiv) and TEA (1.54 g, 3.0 equiv) in DCM (16 mL) was added benzyl carbonochloridate (1.04 g, 1.2 equiv). The reaction was stirred at 0 °C for 1 hour. The mixture was concentrated and purified by silica gel chromatography (SiO₂, Petroleum ether/Ethyl acetate=100/1 to 10/1) to afford the title compound (1.1 g, 69% yield) as white solid. ¹H NMR (400 MHz, DMSO-d6) δ=7.46-7.23 (m, 5H), 5.07 (s, 2H), 3.91 (br d, J=13.2 Hz, 1H), 3.72 (br d, J=12.0 Hz, 1H), 3.61 (s, 3H), 3.23-3.06 (m, 1H), 3.02-2.80 (m, 1H), 2.71 (td, J=4.8, 10.0 Hz, 1H), 2.15 (br dd, J=3.6, 6.8 Hz, 1H), 1.73-1.50 (m, 2H), 0.75 (d, J=6.8 Hz, 3H).

Step B. 1-benzyl 4-methyl (3R,4R)-3-methylpiperidine-1,4-dicarboxylate: methyl (3R,4R)-3-methylpiperidine-4-carboxylate (1.3 g, 1.0 equiv) was purified by SFC separation [column: DAICEL CHIRALPAK AD, 250 mm×50 mm, 10 μm; A: CO₂, B: MeOH (0.2% NH₃H₂O), B %: 15% B over 6.3 min] to afford two isomers.

peak 1: 1-benzyl 4-methyl (3R,4R)-3-methylpiperidine-1,4-dicarboxylate (700 mg, 50% yield) as white solid; LCMS (ESI, M−44): m/z=248.4; SFC>99% ee, column: Chiralpak AD-350×4.6 mm I.D., 3 μm; mobile phase: 5% to 40% MeOH (0.05% DEA) in CO₂, flow rate: 3 mL/min, detector: PDA, t_{R}: 1.054 min.

peak 2: 1-benzyl 4-methyl (3S,4S)-3-methylpiperidine-1,4-dicarboxylate (700 mg, 51% yield) as white solid; LCMS (ESI, M−44): m/z=248.4; SFC>99% ee, column: Chiralpak AD-350×4.6 mm I.D., 3 μm; mobile phase: 5% to 40% MeOH (0.05% DEA) in CO₂, flow rate: 3 mL/min, detector: PDA, t_{R}: 1.162 min.

Step C. methyl (3R,4R)-3-methylpiperidine-4-carboxylate: To a solution of 1-benzyl 4-methyl (3R,4R)-3-methylpiperidine-1,4-dicarboxylate (600 mg, 1.0 equiv) in MeOH (12 mL) was added Pd/C (60 mg, 10% purity). The reaction was degassed and purged with nitrogen 3 times. The reaction was stirred at 25 °C for 2 hours under H₂ atmosphere (15 Psi). The mixture was filtered and concentrated to afford the title compound (300 mg, crude) as yellow oil; ¹H NMR (400 MHz, DMSO-d₆) δ=3.58 (s, 3H), 2.87 (td, J=4.0, 12.2 Hz, 1H), 2.66 (br dd, J=3.6, 6.0 Hz, 2H), 2.56 (td, J=4.4, 10.8 Hz, 1H), 2.45-2.34 (m, 1H), 2.03-1.91 (m, 1H), 1.66-1.38 (m, 2H), 0.85 (d, J=6.8 Hz, 3H).

Step D. methyl (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (150 mg, 1.0 equiv), methyl (3R,4R)-3-methylpiperidine-4-carboxylate (47.8 mg, 1.2 equiv) in DMAC (2 mL) were added K₃PO₄ (161 mg, 3.0 equiv) and 4 Å molecular sieve (30.0 mg). The reaction was stirred at 60 °C for 0.5 hours. The mixture was filtered and filtrate was diluted with H₂O (20 mL) and extracted with ethyl acetate (2×20 mL). The combined organic layers were washed with brine (10 mL), dried over anhydrous sodium sulfate, concentrated to afford the title compound (200 mg, crude) as yellow solid; LCMS (ESI, M+1): m/z=650.4.

Step E. (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylic acid: To a solution of methyl (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylate (200 mg, 1.0 equiv) in MeOH (2 mL) were added LiOH·H₂O (2 M, 2.00 mL, 13 equiv). The reaction was stirred at 25 °C for 0.5 hours. The mixture was purified by reversed phase flash chromatography [C18, 0.1% formic acid condition] to afford the title compound (100 mg, 44% yield) as white solid; LCMS (ESI, M+1): m/z=636.5.

Step F. ((3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-4-yl)(3-methyl-1H-pyrazol-1-yl)methanone: To a solution of (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylic acid (90.0 mg, 1.0 equiv) and HATU (161 mg, 3.0 equiv) in DMF (1 mL) were added 3-methyl-1H-pyrazole (17.4 mg, 1.5 equiv) and DIEA (91.5 mg, 5.0 equiv). The reaction was stirred at 25 °C for 1 hour. The mixture was filtered and purified by prep-HPLC [Waters Xbridge 150×25 mm×5 μm; A: water (NH₄HCO₃); B: ACN, B %: 55%-85% over 9 min] to afford the title compound (10 mg, 10% yield) as white solid; ¹H NMR (400 MHz, CHLOROFORM-d) δ=9.00 (br d, J=4.0 Hz, 1H), 8.22-8.13 (m, 1H), 7.64-7.50 (m, 1H), 7.23-7.16 (m, 2H), 7.13-6.95 (m, 1H), 6.36-6.18 (m, 1H), 5.48-5.12 (m, 1H), 4.49-4.23 (m, 3H), 4.21-4.13 (m, 1H), 3.91 (br dd, J=2.4, 12.8 Hz, 1H), 3.83-3.65 (m, 1H), 3.46-3.09 (m, 3H), 3.06-2.91 (m, 1H), 2.75-2.57 (m, 1H), 2.56-2.40 (m, 1H), 2.39-2.29 (m, 4H), 2.24 (br s, 1H), 2.14 (br dd, J=3.8, 8.2 Hz, 2H), 2.00-1.84 (m, 6H), 1.04-0.92 (m, 3H), 0.82 (q, J=7.6 Hz, 3H); LCMS (ESI, M+1): m/z=700.5.

### Example 80

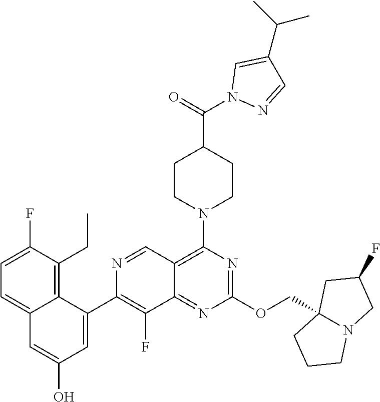 [SMILES: CCc1c(F)ccc2cc(O)cc(-c3ncc4c(N5CCC(C(=O)n6cc(C(C)C)cn6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(4-isopropyl-1H-pyrazol-1-yl)methanone

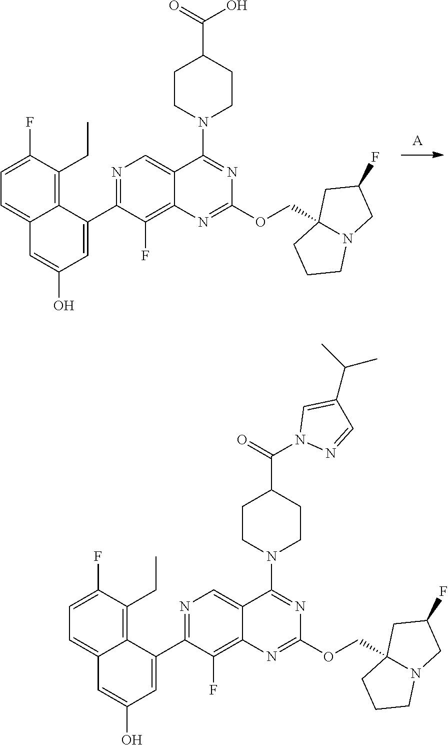

Step A. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(4-isopropyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (80.0 mg, 1.0 equiv) and 4-isopropyl-1H-pyrazole (42.5 mg, 3.0 equiv) in DMF (1 mL) was added HATU (73.4 mg, 1.5 equiv) and DIEA (49.9 mg, 3.0 equiv). The reaction was stirred at 25 °C for 0.5 hours. The mixture was filtered and purified by prep-HPLC [column: Waters Xbridge 150×25 mm×5 μm; A: water (NH₄HCO₃), B: ACN, B %: 62%-92% over 15 min]. The aqueous was extracted with ethyl acetate (2×10 mL). The combined organic layers were dried over anhydrous sodium sulfate and concentrated to afford the title compound (4.79 mg, 15% yield) as white solid; ¹H NMR (400 MHz, CHLOROFORM-d) δ=8.99 (s, 1H), 8.01 (s, 1H), 7.64 (s, 1H), 7.58 (dd, J=5.6, 9.2 Hz, 1H), 7.24-7.18 (m, 2H), 7.05 (dd, J=2.4, 18.4 Hz, 1H), 5.43-5.20 (m, 1H), 4.73-4.58 (m, 2H), 4.35-4.27 (m, 2H), 4.10-3.98 (m, 1H), 3.58-3.46 (m, 2H), 3.41-3.14 (m, 3H), 3.08-2.95 (m, 1H), 2.91-2.83 (m, 1H), 2.57-2.44 (m, 1H), 2.39-2.30 (m, 1H), 2.27-2.08 (m, 7H), 2.04-1.92 (m, 3H), 1.26 (d, J=6.8 Hz, 6H), 0.83 (t, J=6.8 Hz, 3H); LCMS (ESI, M+1): m/z=714.4.

### Example 81

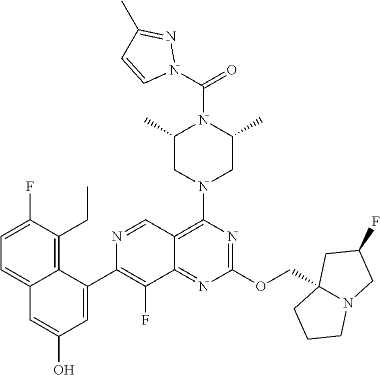 [SMILES: CCc1c(F)ccc2cc(O)cc(-c3ncc4c(N5C[C@@H](C)N(C(=O)n6ccc(C)n6)[C@@H](C)C5)nc(OC[C@@]56CCCN5C[C@H](F)C6)nc4c3F)c12]

### ((2S,6R)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone

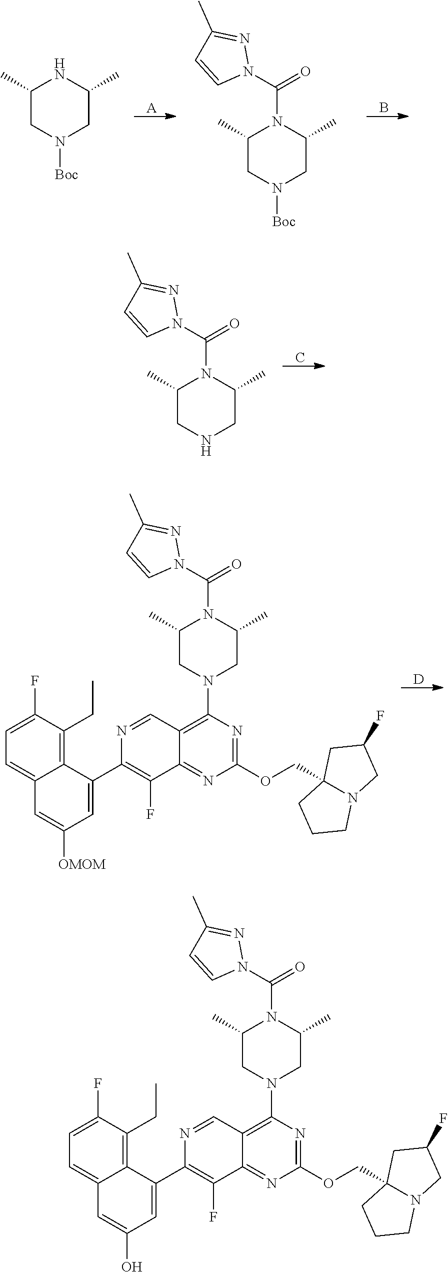

Step A. tert-butyl (3R,5S)-3,5-dimethyl-4-(3-methyl-1H-pyrazole-1-carbonyl)piperazine-1-carboxylate: To a solution of 3-methyl-1H-pyrazole (192 mg, 1.0 equiv) in DCM (3 mL) were added bis(trichloromethyl) carbonate (410 mg, 0.59 equiv) and TEA (472 mg, 2.0 equiv) at 0 °C slowly. The reaction was stirred at 0 °C for 2 hours. tert-butyl (3R,5S)-3,5-dimethylpiperazine-1-carboxylate (500 mg, 1.0 equiv) and TEA (472 mg, 2.0 equiv) in DCM (3 mL) were added at 0 °C. The reaction was stirred at 40 °C for 2 hours. The mixture was poured into water (10 mL) and extracted with DCM (3×10 mL). The combined organic layer was dried over anhydrous Na₂SO₄, filtered, concentrated and purified by column chromatography (SiO₂, petroleum ether/ethyl acetate=20/1 to 1/1) to afford the title compound (500 mg, 88%) as white oil; ¹H NMR (400 MHz, CHLOROFORM-d) δ=8.00 (d, J=2.4 Hz, 1H), 6.14 (d, J=2.4 Hz, 1H), 4.82 (br d, J=2.4 Hz, 2H), 4.06-3.81 (m, 2H), 3.29-3.03 (m, 2H), 2.30 (s, 3H), 1.50 (s, 9H), 1.38 (d, J=7.2 Hz, 6H); LCMS (ESI, M+23): m/z=345.1.

Step B ((2R,6S)-2,6-dimethylpiperazin-1-yl)(3-methyl-1H-pyrazol-1-yl)methanone: To a solution of tert-butyl (3R,5S)-3,5-dimethyl-4-(3-methyl-1H-pyrazole-1-carbonyl)piperazine-1-carboxylate (400 mg, 1.0 equiv) in HCl·MeOH (2 M, 8.00 mL, 12.9 equiv) was stirred at 25 °C for 1 hour. The mixture was concentrated, dissolved with MeOH (5 mL), adjusted to pH=7 with NaHCO₃solid, concentrated under reduced pressure to dryness. The mixture was dissolved with DCM (5 mL) and filtered. The filtrate was concentrated under reduced pressure to dryness to afford the title compound (300 mg, crude) as white oil; LCMS (ESI, M+1): m/z=223.2.

Step C. ((2R,6S)-4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (600 mg, 1.0 equiv) in DMF (6 mL) were added PYBOP (845 mg, 1.5 equiv) and DIEA (420 mg, 3.0 equiv). The reaction was stirred at 25 °C for 0.5 hours. ((2R,6S)-2,6-dimethylpiperazin-1-yl)(3-methyl-1H-pyrazol-1-yl)methanone (289 mg, 1.2 equiv) was added into the mixture. The reaction was stirred at 25 °C for 1.5 hours. The reaction mixture was quenched by addition of water (15 mL) at 0 °C and extracted with ethyl acetate (3×10 mL). The combined organic layer was washed with saturated brine (3×20 mL), dried over anhydrous sodium sulfate, filtered, concentrated and purified by prep-HPLC [Waters Xbridge Prep OBD C18 150×40 mm×10 μm; A: water (NH₄HCO₃), B %: ACN, B %: 55%-85% over 20 min] to afford the title compound (500 mg, 99% yield) as white solid; LCMS (ESI, M+1): m/z=759.4.

Step D. ((2R,6S)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone: A solution of ((2R,6S)-4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone (150 mg, 1.0 equiv) in HCl·MeOH (2 M, 3.00 mL, 30 equiv) was stirred at 25 °C for 1 hour. The mixture was concentrated under reduced pressure. The residue was dissolved with MeOH (3 mL), adjusted to pH=7 with NaHCO₃solid, filtered, concentrated and purified by prep-HPLC [Waters Xbridge 150×25 mm×5 μm; A: water (NH₄HCO₃), B %: ACN, B %: 52%-82% over 9 min] to afford the title compound (55.3 mg, 96% yield) as white solid; ¹H NMR (400 MHz, DMSO-d6) δ=9.92 (s, 1H), 9.22 (s, 1H), 8.13 (d, J=2.4 Hz, 1H), 7.77 (dd, J=6.0, 9.2 Hz, 1H), 7.43-7.29 (m, 2H), 7.02 (d, J=2.4 Hz, 1H), 6.33 (d, J=2.8 Hz, 1H), 5.42-5.13 (m, 1H), 4.66 (br s, 2H), 4.36-4.21 (m, 2H), 4.20-4.14 (m, 1H), 4.12-3.98 (m, 3H), 3.15-2.96 (m, 3H), 2.88-2.76 (m, 1H), 2.40-2.31 (m, 1H), 2.25 (s, 3H), 2.18-1.98 (m, 4H), 1.77 (br s, 3H), 1.54-1.37 (m, 6H), 0.73 (t, J=7.2 Hz, 3H); LCMS (ESI, M+1): m/z=715.4.

### Example 82

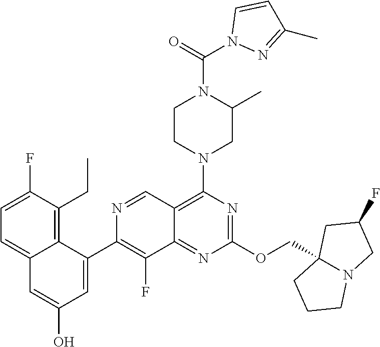 [SMILES: CCc1c(F)ccc2cc(O)cc(-c3ncc4c(N5CCN(C(=O)n6ccc(C)n6)C(C)C5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-methylpiperazin-1-yl)(3-methyl-1H-pyrazol-1-yl)methanone

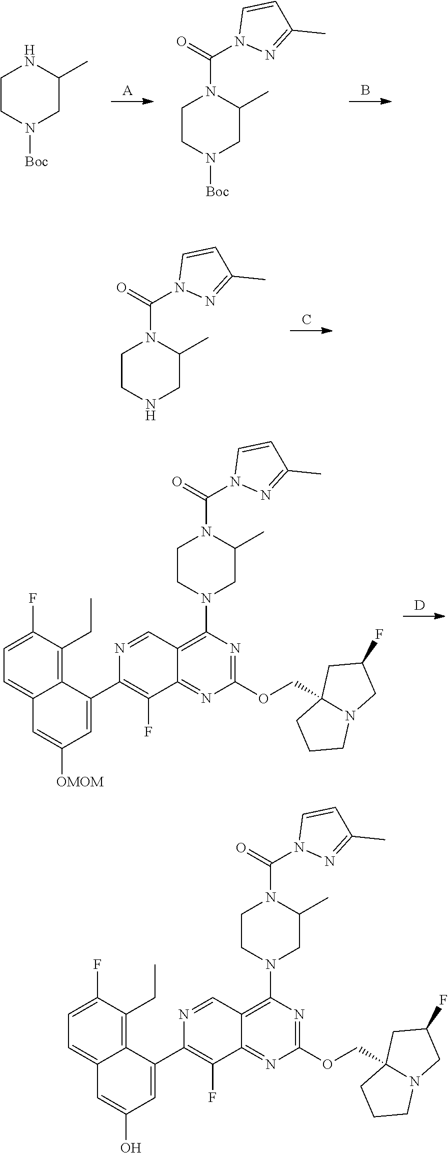

Step A. tert-butyl 3-methyl-4-(3-methyl-1H-pyrazole-1-carbonyl)piperazine-1-carboxylate: To a solution of 3-methyl-1H-pyrazole (205 mg, 1.0 equiv) in DCM (3 mL) was added bis(trichloromethyl) carbonate (490 mg, 0.66 equiv) and TEA (505 mg, 2.0 equiv) at 0 °C. The reaction was stirred at 0 °C for 3 hours. Tert-butyl 3-methylpiperazine-1-carboxylate (500 mg, 1.0 equiv) and TEA (505 mg, 2.0 equiv) in DCM (3 mL) were added at 0 °C The reaction was stirred at 40 °C for 2 hours. The mixture was quenched with water (5 mL) and extracted with dichloromethane (3×10 mL). The organic layers were dried over anhydrous Na₂SO₄, filtered, concentrated and purified by column chromatography (SiO₂, petroleum ether/ethyl acetate=1/0 to 3/1) to afford the title compound (280 mg, 34% yield) as colorless oil; ¹H NMR (400 MHz, CHLOROFORM-d) δ=8.05-7.92 (m, 1H), 6.13 (d, J=2.8 Hz, 1H), 4.78 (br s, 1H), 4.41 (br d, J=12.4 Hz, 1H), 4.18-4.03 (m, 1H), 3.95-3.74 (m, 1H), 3.32 (dt, J=3.6, 12.0 Hz, 1H), 3.21-2.88 (m, 2H), 2.28 (s, 3H), 1.46 (s, 9H), 1.30 (d, J=6.8 Hz, 3H); LCMS (ESI, M+23): m/z=331.1.

Step B. (3-methyl-1H-pyrazol-1-yl)(2-methylpiperazin-1-yl)methanone; A solution of tert-butyl 3-methyl-4-(3-methyl-1H-pyrazole-1-carbonyl)piperazine-1-carboxylate (230 mg, 1.0 equiv) in HCl·MeOH (2 M, 4.6 mL, 12.3 equiv) was stirred at 25 °C for 2 hours. The mixture was concentrated, diluted with MeOH (3 mL), adjusted to pH about 7 with NaHCO₃solid, filtered and concentrated to afford the title compound (153 mg, 97% yield) as white solid; LCMS (ESI, M+1): m/z=209.2.

Step C. (4-(7-(8-ethyl-7-fluoro-3-(methoxymethoxy)naphthalen-1-yl)-8-fluoro-2-yl)-2-methylpiperazin-1-yl)(3-methyl-1H-pyrazol-1-yl)methanone: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (380 mg, 1.0 equiv) in DMF (6 mL) were added DIEA (266 mg, 3.0 equiv) and PyBOP (535 mg, 1.5 equiv) slowly at 25 °C, the reaction was stirred at 25 °C for 20 minutes. (3-methyl-1H-pyrazol-1-yl)(2-methylpiperazin-1-yl)methanone (150 mg, 1.0 equiv) was added at 25 °C slowly. The reaction was stirred at 25 °C for 2 hours. The mixture was filtered and purified by reversed-phase flash (0.1% FA condition) to afford the title compound (700 mg, crude, HCOOH) as colorless oil; LCMS (ESI, M+1): m/z=745.4.

Step D. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-methylpiperazin-1-yl)(3-methyl-1H-pyrazol-1-yl)methanone: A solution of (4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-methylpiperazin-1-yl)(3-methyl-1H-pyrazol-1-yl)methanone (300 mg, 1.0 equiv, HCOOH) in HCl·MeOH (2 M, 5 mL, 26 equiv) was stirred at 25 °C for 2 hours. The mixture was concentrated, diluted with MeOH (3 mL), adjusted to pH about 7 with NaHCO₃solid, filtered, concentrated and purified by prep-HPLC [Waters Xbridge Prep OBD C18 150×40 mm×10 μm; A: water (NH₄HCO₃), B: ACN, B %: 40%-70% over 20 min] to afford the title compound (81.0 mg, 30% yield over two steps) as white solid; ¹H NMR (400 MHz, DMSO-d6) δ=10.04-9.82 (m, 1H), 9.20 (s, 1H), 8.13 (t, J=2.4 Hz, 1H), 7.77 (dd, J=6.0, 9.2 Hz, 1H), 7.43-7.28 (m, 2H), 7.13-6.94 (m, 1H), 6.33 (d, J=2.8 Hz, 1H), 5.44-5.16 (m, 1H), 4.85-4.65 (m, 1H), 4.51-4.39 (m, 1H), 4.39-4.25 (m, 2H), 4.21-3.97 (m, 3H), 3.96-3.83 (m, 2H), 3.30-3.24 (m, 1H), 3.15-3.06 (m, 2H), 2.89-2.74 (m, 1H), 2.43-2.32 (m, 1H), 2.25 (s, 3H), 2.19-1.96 (m, 4H), 1.77 (br s, 3H), 1.37 (dd, J=6.8, 10.0 Hz, 3H), 0.79-0.65 (m, 3H); LCMS (ESI, M+1): m/z=701.3.

### Example 83

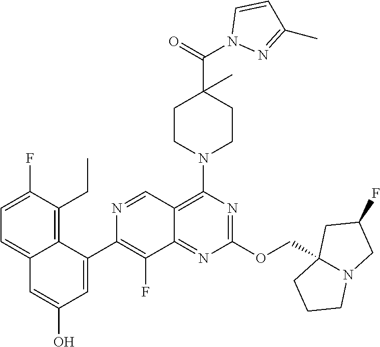 [SMILES: CCc1c(F)ccc2cc(O)cc(-c3ncc4c(N5CCC(C)(C(=O)n6ccc(C)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidin-4-yl)(3-methyl-1H-pyrazol-1-yl)methanone

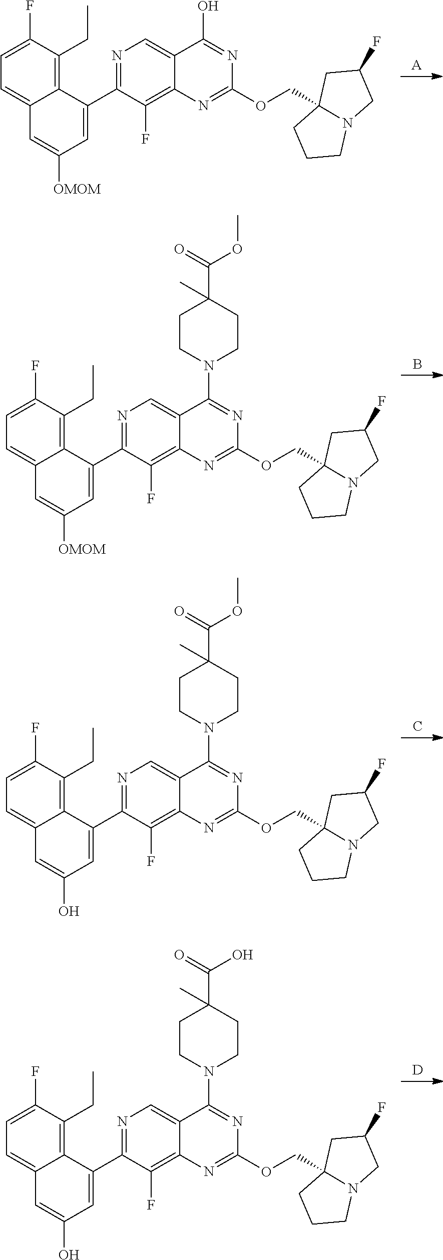

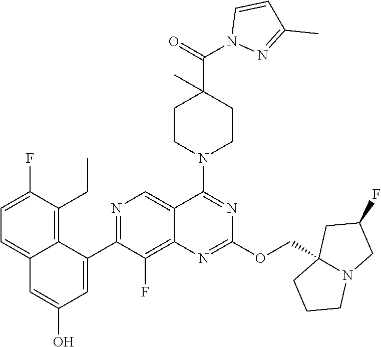 [SMILES: CCc1c(F)ccc2cc(O)cc(-c3ncc4c(N5CCC(C)(C(=O)n6ccc(C)n6)CC5)nc(OC[C@@]56CCCN5C[C@H](F)C6)nc4c3F)c12]

Step A. methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidine-4-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (2.3 g, 1.0 equiv) and methyl 4-methylpiperidine-4-carboxylate (1.0 g, 1.2 equiv, HCl salt) in DMF (20 mL) were added PyBOP (3.24 g, 1.5 equiv) and DIEA (5.36 g, 10 equiv). The reaction was stirred at 25 °C for 12 hours. The mixture was filtered and purified by reversed phase flash chromatography [C18, 0.1% formic acid condition] to afford the title compound (3.4 g, 87% yield) as yellow solid; LCMS (ESI, M+1): m/z=694.4.

Step B. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidine-4-carboxylate: To a solution of methyl 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidine-4-carboxylate (3.4 g, 1.0 equiv) in MeOH (15 mL) was added HCl·MeOH (2 M, 30 mL, 12 equiv). The reaction was stirred at 25 °C for 4 hours. The mixture was concentrated. The residue was basified with saturated NaHCO₃ solution (80 mL) and extracted with ethyl acetate (3×50 mL). The combined organic layers were dried over anhydrous sodium sulfate, concentrated and purified by reversed phase flash chromatography [C18, 0.1% NH₃·H₂O condition] to afford the title compound (1.9 g, 55% yield) as white solid; LCMS (ESI, M+1): m/z=650.4.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidine-4-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidine-4-carboxylate (1.9 g, 1.0 equiv) in MeOH (15 mL) was added LiOH·H₂O (2 M in H₂O, 15 mL, 10 equiv). The reaction was stirred at 25 °C for 4 hours. The mixture was filtered and the filtrate was purified by reversed phase flash chromatography [C18, 0.1% formic acid condition] to afford the title compound (1.12 g, 34% yield) as yellow solid; LCMS (ESI, M+1): m/z=636.4.

Step D. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidin-4-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpiperidine-4-carboxylic acid (300 mg, 1.0 equiv) and 3-methyl-1H-pyrazole (58.1 mg, 1.5 equiv) in DMF (3 mL) were added HATU (359 mg, 2.0 equiv) and DIEA (366 mg, 6.0 equiv). The reaction was stirred at 25 °C. for 12 hours. The mixture was filtered purified by prep-HPLC [Waters Xbridge Prep OBD C18 150×40 mm×10 μm; A: water (NH₄HCO₃), B: ACN, B %: 52%-82% over 20 min] to afford the title compound (8.22 mg, 2.4% yield) as yellow solid; ¹H NMR (400 MHz, CHLOROFORM-d) δ=8.93 (d, J=2.1 Hz, 1H), 8.19 (d, J=2.8 Hz, 1H), 7.56-7.48 (m, 1H), 7.20-7.10 (m, 2H), 7.01-6.88 (m, 1H), 6.22 (d, J=2.8 Hz, 1H), 5.40-5.17 (m, 1H), 4.34-4.18 (m, 4H), 3.75-3.59 (m, 2H), 3.36-3.11 (m, 3H), 3.04-2.93 (m, 1H), 2.89-2.76 (m, 2H), 2.50-2.41 (m, 1H), 2.33 (s, 3H), 2.27-2.12 (m, 4H), 1.97-1.89 (m, 5H), 1.64 (br d, J=1.1 Hz, 3H), 0.81 (t, J=7.4 Hz, 3H); LCMS (ESI, M+1): m/z=700.4.

### Example 84

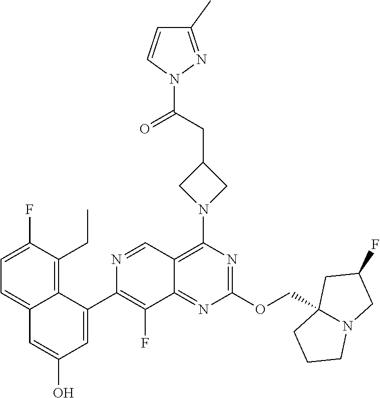 [SMILES: CCc1c(F)ccc2cc(O)cc(-c3ncc4c(N5CC(CC(=O)n6ccc(C)n6)C5)nc(OC[C@@]56CCCN5C[C@H](F)C6)nc4c3F)c12]

### 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)-1-(3-methyl-1H-pyrazol-1-yl) ethan-1-one

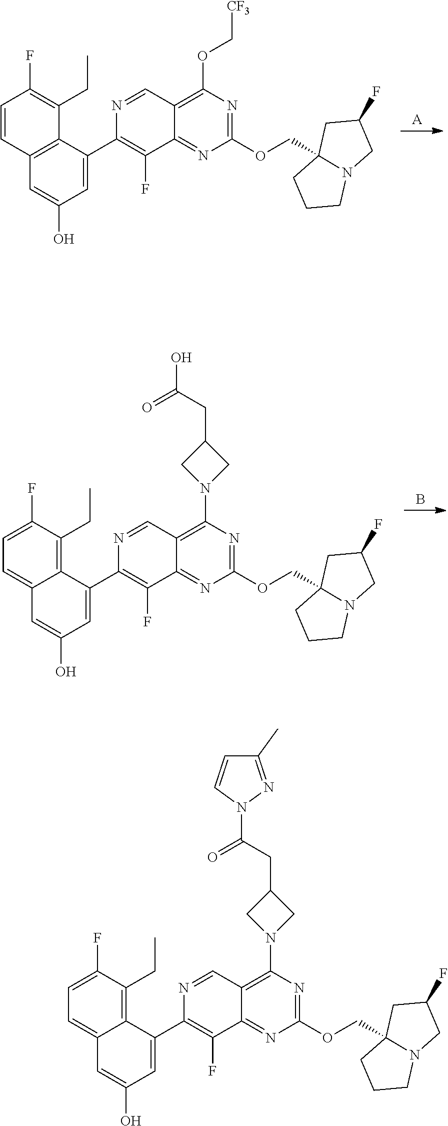

Step A. 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl) acetic acid: To a solution of 2-(azetidin-3-yl) acetic acid (154 mg, 2 equiv, HCl), DIEA (327 mg, 5.0 equiv) in DMF (3.00 mL) was added 4 Å molecular sieve (20 mg). The reaction was stirred at 25 °C for 10 minutes. Then 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) was added. The reaction was stirred at 40 °C for 3 hours. The mixture was filtered and purified by reversed phase flash chromatography [C18, 0.1% formic acid condition] to afford the title compound (320 mg, 96% yield, HCOOH) as white solid; ¹H NMR (400 MHz, DMSO-d6) δ 10.25-9.58 (m, 1H), 8.93 (s, 1H), 7.76 (dd, J=6.0, 9.2 Hz, 1H), 7.41-7.27 (m, 2H), 7.00 (d, J=2.4 Hz, 1H), 5.46-5.22 (m, 1H), 5.06-4.89 (m, 1H), 4.62-4.38 (m, 2H), 4.28-3.96 (m, 4H), 3.23-3.08 (m, 5H), 2.95-2.85 (m, 1H), 2.76 (br d, J=7.2 Hz, 2H), 2.43-2.28 (m, 1H), 2.22-1.98 (m, 4H), 1.94-1.74 (m, 3H), 0.78-0.63 (m, 3H); LCMS (ESI, M+1): m/z=608.3.

Step B. 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)-1-(3-methyl-1H-pyrazol-1-yl) ethan-1-one: To a solution of 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl) acetic acid (100 mg, 1.0 equiv) and HATU (93.9 mg, 1.5 equiv) in DMF (1.00 mL) was added DIEA (106 mg, 5.0 equiv). The reaction was stirred at 25 °C for 0.5 hours. Then 3-methyl-1H-pyrazole (27.0 mg, 2.0 equiv) was added into the reaction mixture. The reaction was stirred at 25 °C for 0.5 hours. The mixture was filtered and purified by prep-HPLC [Waters Xbridge 150×25 mm×5 μm; A: water (NH₄HCO₃), B: ACN; B %: 45%-75% B over 9 min] to afford the title compound (5.05 mg, 4.2% yield) as white solid; ¹H NMR (400 MHz, CHLOROFORM-d) δ=8.79 (s, 1H), 8.15 (d, J=2.8 Hz, 1H), 7.60-7.49 (m, 1H), 7.21-7.09 (m, 2H), 6.97-6.78 (m, 1H), 6.30 (d, J=2.8 Hz, 1H), 5.42-5.18 (m, 1H), 5.03-4.51 (m, 2H), 4.45-4.07 (m, 4H), 3.57-3.14 (m, 6H), 3.09-2.95 (m, 1H), 2.54-2.41 (m, 1H), 2.34 (d, J=1.2 Hz, 3H), 2.31-2.21 (m, 2H), 2.19-2.07 (m, 3H), 1.96 (br s, 2H), 0.80 (t, J=7.2 Hz, 3H); LCMS (ESI, M+1): m/z=672.2.

### Example 85

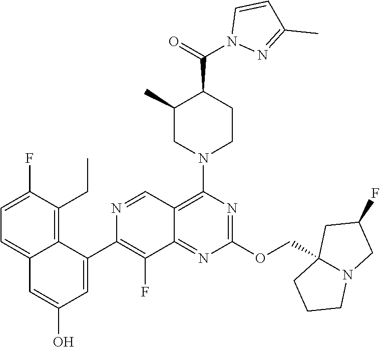 [SMILES: CCc1c(F)ccc2cc(O)cc(-c3ncc4c(N5CC[C@H](C(=O)n6ccc(C)n6)[C@H](C)C5)nc(OC[C@@]56CCCN5C[C@H](F)C6)nc4c3F)c12]

### ((3S,4S)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-4-yl)(3-methyl-1H-pyrazol-1-yl)methanone

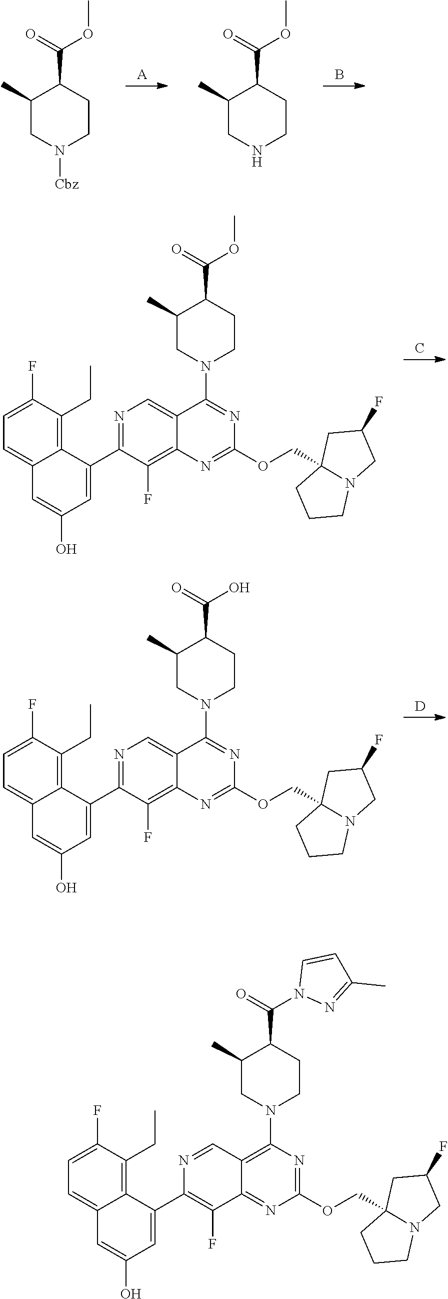

Step A. methyl (3S,4S)-3-methylpiperidine-4-carboxylate: To a solution of 1-benzyl 4-methyl (3S,4S)-3-methylpiperidine-1,4-dicarboxylate (700 mg, 1.0 equiv) in MeOH (14 mL) was added Pd/C (70 mg, 10% purity). The reaction was degassed and purged with nitrogen 3 times and stirred at 25 °C for 2 hours under H₂ atmosphere (15 Psi). The mixture was filtered and concentrated to afford the title compound (350 mg, crude) as yellow oil; ¹H NMR (400 MHz, DMSO-d₆) δ=3.59 (s, 3H), 2.88 (td, J=4.0, 12.0 Hz, 1H), 2.73-2.62 (m, 2H), 2.57 (td, J=4.4, 10.8 Hz, 1H), 2.46-2.39 (m, 1H), 2.00 (tdd, J=3.2, 7.2, 10.4 Hz, 1H), 1.71-1.39 (m, 2H), 0.85 (d, J=7.2 Hz, 3H).

Step B. methyl (3S,4S)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (500 mg, 1.0 equiv) and methyl (3S,4S)-3-methylpiperidine-4-carboxylate (199 mg, 1.5 equiv) in DMF (10 mL) were added K₃PO₄ (537 mg, 3.0 equiv) and 4 Å molecular sieve (100 mg). The reaction was stirred at 60 °C for 0.5 hours. The mixture was filtered, diluted with H₂O (50 mL) and extracted with ethyl acetate (2×20 mL). The combined organic layers were washed with brine (20 mL), dried over anhydrous sodium sulfate, concentrated to afford the title compound (400 mg, crude) as yellow solid; LCMS (ESI, M+1): m/z=650.5.

Step C. (3S,4S)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylic acid: To a solution of methyl (3S,4S)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylate (400 mg, 1.0 equiv) in MeOH (4 mL) were added LiOH·H₂O (2 M, 4.00 mL, 13 equiv). The reaction was stirred at 25 °C for 1 hour. The mixture was filtered and purified by reversed phase flash chromatography [C18, 0.1% formic acid condition] to afford the title compound (250 mg, 63% yield) as white solid; LCMS (ESI, M+1): m/z=636.3.

Step D. ((3S,4S)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-4-yl)(3-methyl-1H-pyrazol-1-yl)methanone: To a solution of (3S,4S)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidine-4-carboxylic acid (250 mg, 1.0 equiv) and HATU (449 mg, 3.0 equiv) in DMF (3 mL) were added 3-methyl-1H-pyrazole (17.4 mg, 1.5 equiv) and DIEA (254 mg, 5.0 equiv). The reaction was stirred at 25 °C for 1 hour. The mixture was filtered and purified by prep-HPLC [Waters Xbridge Prep OBD C18 150×40 mm×10 μm; A: water (NH₄HCO₃); B: ACN, B %: 46%-76% over 20 min] to afford the title compound (11 mg, 4% yield) as white solid; ¹H NMR (400 MHz, CHLOROFORM-d) δ=9.00 (s, 1H), 8.17 (t, J=3.2 Hz, 1H), 7.59-7.51 (m, 1H), 7.24-7.16 (m, 2H), 7.09-6.99 (m, 1H), 6.29 (t, J=2.2 Hz, 1H), 5.41-5.18 (m, 1H), 4.47-4.25 (m, 3H), 4.22-4.12 (m, 1H), 3.97-3.84 (m, 1H), 3.82-3.65 (m, 1H), 3.35-3.13 (m, 3H), 3.05-2.95 (m, 1H), 2.70-2.59 (m, 1H), 2.55-2.42 (m, 1H), 2.38-2.28 (m, 4H), 2.23 (br s, 1H), 2.20-2.09 (m, 2H), 2.05-1.82 (m, 6H), 1.08-0.91 (m, 3H), 0.89-0.75 (m, 3H); LCMS (ESI, M+1): m/z=700.3.

### Example 86

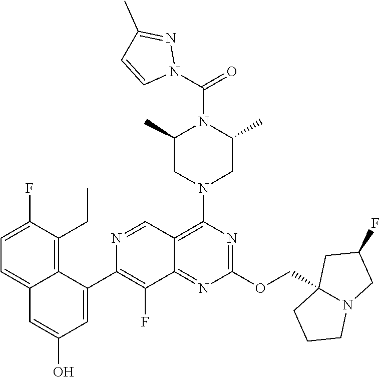 [SMILES: CCc1c(F)ccc2cc(O)cc(-c3ncc4c(N5C[C@@H](C)N(C(=O)n6ccc(C)n6)[C@H](C)C5)nc(OC[C@@]56CCCN5C[C@H](F)C6)nc4c3F)c12]

### ((2R,6R)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone

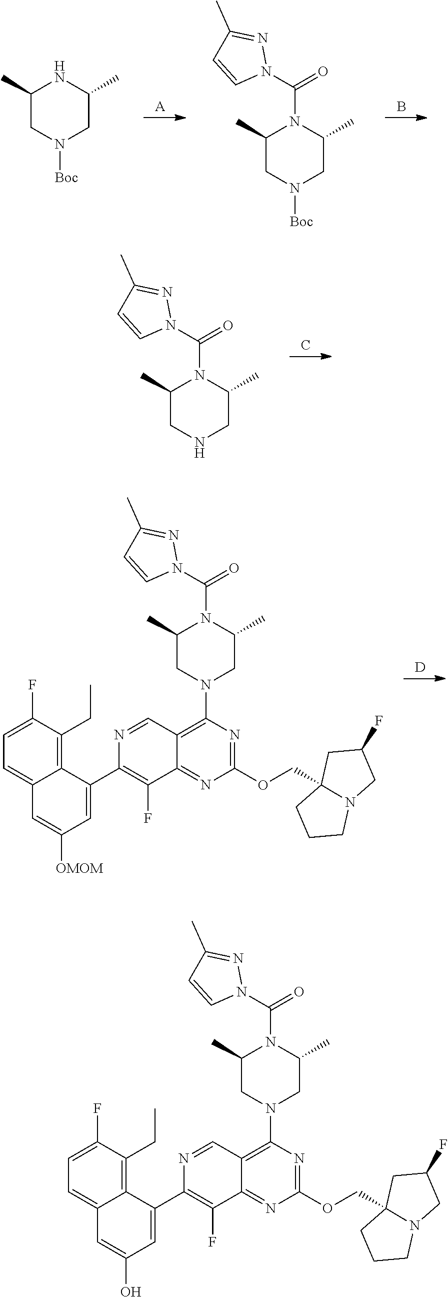

Step A. tert-butyl (3R,5R)-3,5-dimethyl-4-(3-methyl-1H-pyrazole-1-carbonyl)piperazine-1-carboxylate: To a solution of 3-methyl-1H-pyrazole (190 mg, 1.0 equiv) and TEA (468 mg, 2.0 equiv) in DCM (3 mL) was added bis(trichloromethyl) carbonate (130 mg, 0.19 equiv) at 0 °C. The reaction was stirred at 0 °C for 2 hours. Tert-butyl (3R,5R)-3,5-dimethylpiperazine-1-carboxylate (496 mg, 1.0 equiv) and TEA (468 mg, 2.0 equiv) in DCM (2 mL) were added. The reaction was stirred at 25 °C for 2 hours. The mixture was quenched with water (5 mL) and extracted with dichloromethane (3×10 mL). The organic layers were dried over anhydrous Na₂SO₄, filtered, concentrated and purified by column chromatography (SiO₂, petroleum ether/ethyl acetate=1/0 to 5/1) to afford the title compound (60.0 mg, 7.6% yield) as white solid; ¹H NMR (400 MHz, CHLOROFORM-d) δ=8.08 (d, J=2.8 Hz, 1H), 6.15 (d, J=2.8 Hz, 1H), 4.81 (br s, 2H), 3.69-3.54 (m, 4H), 2.31 (s, 3H), 1.49 (s, 9H), 1.32 (d, J=6.8 Hz, 6H); LCMS (ESI, M+1): m/z=323.4.

Step B. ((2R,6R)-2,6-dimethylpiperazin-1-yl)(3-methyl-1H-pyrazol-1-yl)methanone: A solution of tert-butyl (3R,5R)-3,5-dimethyl-4-(3-methyl-1H-pyrazole-1-carbonyl)piperazine-1-carboxylate (50 mg, 1.0 equiv) in HCl·MeOH (2 M, 64 equiv) was stirred at 0 °C for 1 hour. The mixture was concentrated, diluted with MeOH (3 mL), adjusted to pH about 8 with NaHCO₃solid, filtered and concentrated to afford the title compound (50.0 mg, crude) as yellow oil; LCMS (ESI, M+1): m/z=223.1.

Step C. ((2R,6R)-4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone: To a solution of ((2R,6R)-2,6-dimethylpiperazin-1-yl)(3-methyl-1H-pyrazol-1-yl)methanone (40.1 mg, 1.0 equiv) and 7-[8-ethyl-7-fluoro-3-(methoxymethoxy)-1-naphthyl]-8-fluoro-2-[[(2R,8S)-2-fluoro-1,2,3,5,6,7-hexahydropyrrolizin-8-yl]methoxy]pyrido[4,3-d]pyrimidin-4-ol (100 mg, 1.0 equiv) in DMF (1 mL) were added DIEA (93.2 mg, 4.0 equiv) and HATU (137 mg, 2.0 equiv). The reaction was stirred at 25 °C for 0.5 hours. The mixture was filtered and purified by prep-HPLC [C18 150×30 mm; A: water(FA), B: ACN, B %: 25%-55% over 7 min] to afford the title compound (60.0 mg, 38% yield, HCOOH) as brown solid; LCMS (ESI, M+1): m/z=759.4.

Step D. ((2R,6R)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone: A solution of ((2R,6R)-4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,6-dimethylpiperazin-1-yl)(3-methyl-1H-pyrazol-1-yl)methanone (50.0 mg, 1.0 equiv, HCOOH) in HCl·MeOH (2 M, 121 equiv) was stirred at 25 °C for 0.5 hours. The mixture was concentrated, diluted with MeOH (3 mL), adjusted to pH about 8 with NaHCO₃solid, filtered, concentrated and purified by prep-HPLC [Phenomenex luna C18 150×25 mm×10 μm; A: water(FA), B: ACN, B %: 25%-55% over 9 min] and prep-HPLC [Waters Xbridge 150×25 mm×5 μm; A: water (NH₄HCO₃); B: ACN, B %: 52%-82% over 9 min] to afford the title compound (10.0 mg, 34% yield) as white solid; ¹H NMR (400 MHz, CHLOROFORM-d) 8=9.07 (s, 1H), 8.16 (d, J=2.0 Hz, 1H), 7.56 (td, J=6.4, 9.2 Hz, 1H), 7.24-7.14 (m, 2H), 7.08-6.93 (m, 1H), 6.18 (t, J=2.8 Hz, 1H), 5.48-5.15 (m, 3H), 4.48-4.29 (m, 3H), 4.28-4.18 (m, 1H), 4.14-3.98 (m, 2H), 3.33-3.08 (m, 3H), 3.04-2.92 (m, 1H), 2.61-2.40 (m, 1H), 2.32 (d, J=7.6 Hz, 4H), 2.10 (br t, J=8.4 Hz, 3H), 2.03-1.87 (m, 3H), 1.43 (dd, J=4.8, 6.4 Hz, 6H), 0.83 (q, J=7.6 Hz, 3H); LCMS (ESI, M+1): m/z=715.4.

### Example 87

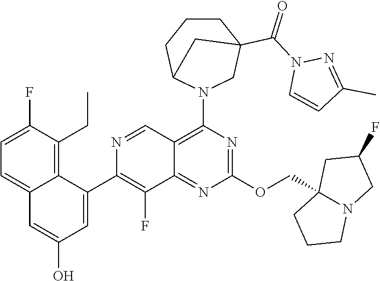 [SMILES: CCc1c(F)ccc2cc(O)cc(-c3ncc4c(N5CC6(C(=O)n7ccc(C)n7)CCCC5C6)nc(OC[C@@]56CCCN5C[C@H](F)C6)nc4c3F)c12]

### (6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-azabicyclo[3.2.1]octan-1-yl)(3-methyl-1H-pyrazol-1-yl)methanone

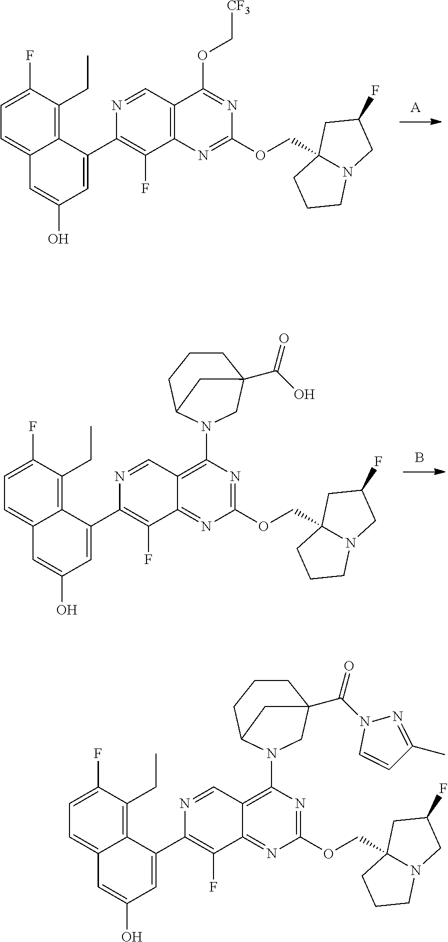

Step A. 6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-azabicyclo[3.2.1]octane-1-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and 6-azabicyclo[3.2.1]octane-1-carboxylic acid (194 mg, 2.0 equiv, HCl) in DMF (2.5 mL) were added DIEA (261 mg, 4.0 equiv) and K₃PO₄ (322 mg, 3.0 equiv). The reaction was stirred at 60 °C for 0.5 hours. The mixture was filtered and purified by reversed phase flash chromatography [C18, 0.1% formic acid condition] to afford the title compound (210 mg, 63% yield, HCOOH salt) as white solid; LCMS (ESI, M+1): m/z=648.3.

Step B. (6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-azabicyclo[3.2.1]octan-1-yl)(3-methyl-1H-pyrazol-1-yl)methanone: To a solution of 6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6-azabicyclo[3.2.1]octane-1-carboxylic acid (50 mg, 1.0 equiv) and 3-methyl-1H-pyrazole (31.7 mg, 5 equiv) in DMF (0.5 mL) were added HATU (58.7 mg, 2.0 equiv) and DIEA (59.9 mg, 6.0 equiv). The reaction was stirred at 25 °C for 20 mins. The mixture was filtered and purified by reversed phase flash chromatography [C18, water/ACN] to afford the title compound (4.77 mg, 8% yield) as white solid; ¹H NMR (400 MHz, CHLOROFORM-d) 8-9.38-9.20 (m, 1H), 8.21-8.09 (m, 1H), 7.58-7.46 (m, 1H), 7.22-6.98 (m, 3H), 6.25 (br s, 1H), 5.46-5.04 (m, 2H), 4.92-4.72 (m, 1H), 4.42-4.18 (m, 3H), 3.42-3.13 (m, 3H), 3.05-2.91 (m, 1H), 2.46 (br d, J=11.2 Hz, 3H), 2.36-2.22 (m, 6H), 2.12 (br d, J=2.0 Hz, 1H), 2.02-1.91 (m, 5H), 1.80-1.60 (m, 4H), 0.89-0.72 (m, 3H); LCMS (ESI, M+1): m/z=712.4.

### Example 88

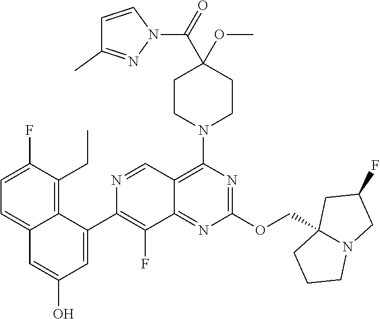 [SMILES: CCc1c(F)ccc2cc(O)cc(-c3ncc4c(N5CCC(OC)(C(=O)n6ccc(C)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methoxypiperidin-4-yl)(3-methyl-1H-pyrazol-1-yl)methanone

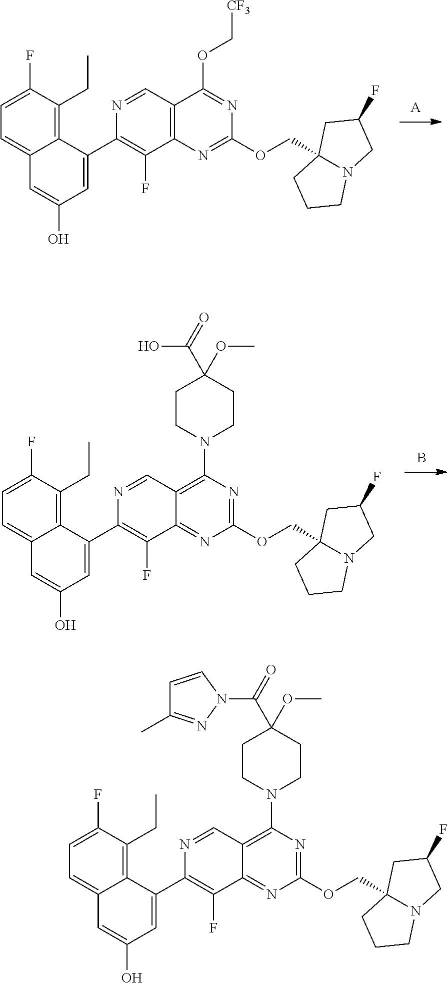

Step A. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methoxypiperidine-4-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and 4-methoxypiperidine-4-carboxylic acid (198 mg, 2.0 equiv, HCl) in DMF (2 mL) were added DIEA (262 mg, 4.0 equiv) and K₃PO₄ (322 mg, 3.0 equiv). The reaction was stirred at 60 °C for 0.5 hours. The mixture was filtered and purified by reversed phase flash chromatography [C18, 0.1% formic acid condition] to afford the title compound (240 mg, 72% yield, HCOOH salt) as yellow solid; LCMS (ESI, M+1): m/z=652.3.

Step B. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methoxypiperidin-4-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methoxypiperidine-4-carboxylic acid (70 mg, 1.0 equiv) and 3-methyl-1H-pyrazole (44.1 mg, 5.0 equiv) in DMF (0.7 mL) were added HATU (81.7 mg, 2.0 equiv) and DIEA (55.5 mg, 4.0 equiv). The reaction was stirred at 30 °C for 20 mins. The mixture was filtered and purified by reversed phase flash chromatography [C18, water/ACN] to afford the title compound (14.4 mg, 18% yield) as white solid; ¹H NMR (400 MHz, CHLOROFORM-d) δ=9.00 (d, J=2.4 Hz, 1H), 8.32 (d, J=2.8 Hz, 1H), 7.64-7.51 (m, 1H), 7.25-7.14 (m, 2H), 7.05-6.93 (m, 1H), 6.29 (d, J=3.2 Hz, 1H), 5.43-5.19 (m, 1H), 4.45-4.26 (m, 4H), 3.92-3.74 (m, 2H), 3.37-3.28 (m, 4H), 3.27-3.17 (m, 1H), 3.06-2.98 (m, 1H), 2.60-2.42 (m, 5H), 2.38 (s, 3H), 2.36-2.30 (m, 1H), 2.29-2.13 (m, 3H), 2.03-1.86 (m, 4H), 0.84 (t, J=7.6 Hz, 3H); LCMS (ESI, M+1): m/z=716.3.

### Example 89

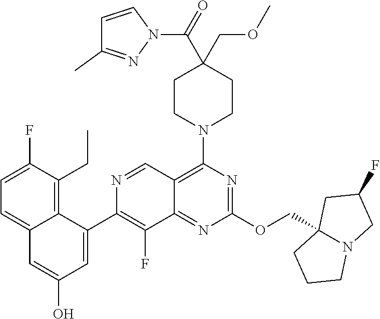 [SMILES: CCc1c(F)ccc2cc(O)cc(-c3ncc4c(N5CCC(COC)(C(=O)n6ccc(C)n6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-(methoxymethyl)piperidin-4-yl)(3-methyl-1H-pyrazol-1-yl)methanone

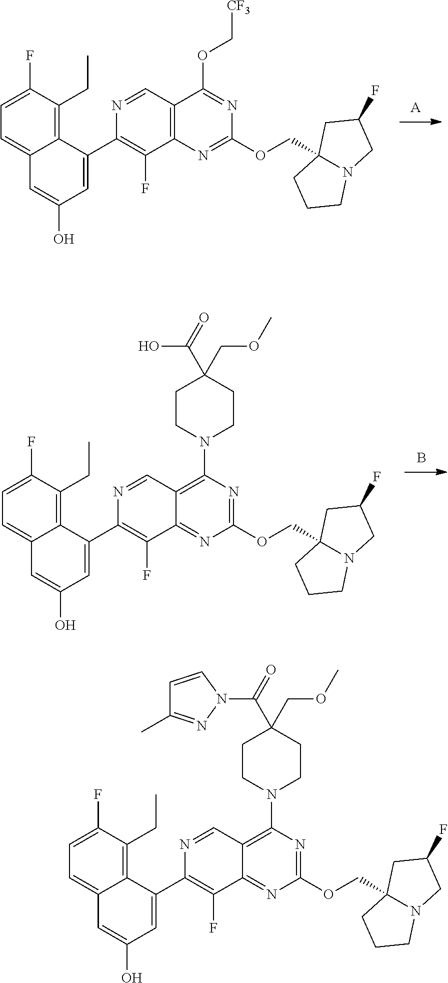

Step A. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-(methoxymethyl)piperidine-4-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and 4-(methoxymethyl)piperidine-4-carboxylic acid (212 mg, 2.0 equiv, HCl) in DMF (2 mL) were added DIEA (262 mg, 4.0 equiv) and K₃PO₄ (322 mg, 3.0 equiv). The reaction was stirred at 60 °C for 20 minutes. The mixture was filtered and purified by reversed phase flash chromatography [C18, 0.1% formic acid condition] to afford the title compound (260 mg, 74% yield, HCOOH salt) as white solid; LCMS (ESI, M+1): m/z=666.3.

Step B. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-(methoxymethyl)piperidin-4-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-(methoxymethyl)piperidine-4-carboxylic acid (100 mg, 1.0 equiv) and 3-methyl-1H-pyrazole (61.7 mg, 5.0 equiv) in DMF (1 mL) were added HATU (114 mg, 2.0 equiv) and DIEA (77.7 mg, 4.0 equiv). The reaction was stirred at 30 °C for 20 minutes. The mixture was filtered and purified by reversed phase flash chromatography [C18, water/ACN] to afford the title compound (7.46 mg, 7% yield) as white solid; ¹H NMR (400 MHz, CHLOROFORM-d) δ=8.97 (s, 1H), 8.23 (d, J=2.8 Hz, 1H), 7.56 (dd, J=6.0, 9.2 Hz, 1H), 7.24-7.15 (m, 2H), 7.01-6.89 (m, 1H), 6.24 (d, J=2.8 Hz, 1H), 5.43-5.19 (m, 1H), 4.38-4.21 (m, 4H), 4.06-3.97 (m, 2H), 3.75-3.59 (m, 2H), 3.42-3.28 (m, 2H), 3.27-3.25 (m, 3H), 3.23-3.12 (m, 1H), 3.06-2.97 (m, 1H), 2.94-2.82 (m, 2H), 2.52-2.41 (m, 1H), 2.35 (s, 3H), 2.31-2.11 (m, 4H), 2.03-1.92 (m, 5H), 0.83 (t, J=7.6 Hz, 3H); LCMS (ESI, M+1): m/z=730.3.

### Example 90

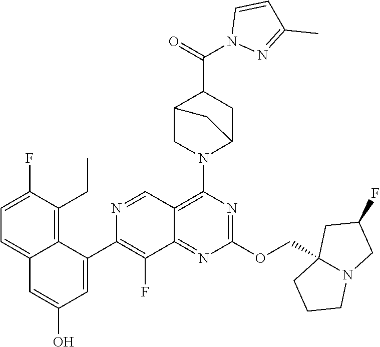 [SMILES: CCc1c(F)ccc2cc(O)cc(-c3ncc4c(N5CC6CC5CC6C(=O)n5ccc(C)n5)nc(OC[C@@]56CCCN5C[C@H](F)C6)nc4c3F)c12]

### (2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[2.2.1]heptan-5-yl)(3-methyl-1H-pyrazol-1-yl)methanone

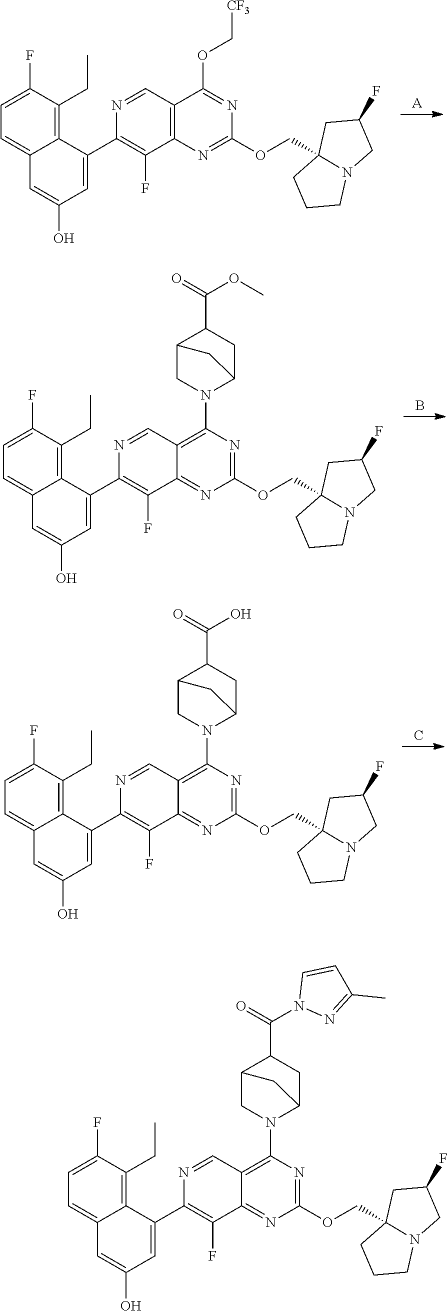

Step A. methyl 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[2.2.1]heptane-5-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (200 mg, 1.0 equiv) and methyl 2-azabicyclo[2.2.1]heptane-5-carboxylate (68.1 mg, 1.3 equiv) in DMAC (1 mL) was added DIEA (131 mg, 3.0 equiv). The reaction was stirred at 30 °C for 2 hours. The mixture was filtered and purified by reversed phase flash [C18, 0.1% formic acid condition] to afford the title compound (160 mg, 60% yield) as yellow solid; LCMS (ESI, M+1): m/z=648.3.

Step B. 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[2.2.1]heptane-5-carboxylic acid: To a solution of methyl 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[2.2.1]heptane-5-carboxylate (150 mg, 1.0 equiv) in MeOH (5 mL) was added LiOH·H₂O (2 M, 0.6 mL, 5.0 equiv). The reaction was stirred at 25 °C for 2 hours. The mixture was concentrated and diluted with water (3 mL). The mixture was adjusted to pH=7 with 0.1 M HCl and filtered to give a residue. The residue was added toluene (10 mL) and concentrated to afford the title compound (145 mg, 98% yield) as white solid; LCMS (ESI, M+1): m/z=634.3.

Step C. (2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[2.2.1]heptan-5-yl)(3-methyl-1H-pyrazol-1-yl)methanone: To a solution of 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[2.2.1]heptane-5-carboxylic acid (140 mg, 1.0 equiv), 3-methyl-1H-pyrazole (27.2 mg, 1.5 equiv) and DIEA (85.7 mg, 3.0 equiv) in DMF (1 mL) was added HATU (126 mg, 1.5 equiv). The reaction was stirred at 25 °C for 1 hour. The mixture was filtered and purified by prep-HPLC [column: Waters Xbridge 150×25 mm×5 μm; A: water (NH₄HCO₃), B: ACN, B %: 45%-65% over 10 minutes] to afford the title compound (23.1 mg, 15% yield) as white solid; ¹H NMR (400 MHz, DMSO-d₆) δ=9.94 (s, 1H), 9.27-9.06 (m, 1H), 8.39-8.11 (m, 1H), 7.83-7.66 (m, 1H), 7.44-7.23 (m, 2H), 7.11-6.91 (m, 1H), 6.46 (br d, J=2.4 Hz, 1H), 5.28 (br d, J=54.4 Hz, 1H), 5.16-5.05 (m, 1H), 4.38-3.82 (m, 4H), 3.75-3.52 (m, 1H), 3.22 (br s, 1H), 3.15-3.01 (m, 3H), 2.90-2.73 (m, 1H), 2.54 (s, 2H), 2.35-2.23 (m, 4H), 2.21-1.97 (m, 6H), 1.90-1.72 (m, 3H), 0.88-0.59 (m, 3H); LCMS (ESI, M+1): m/z=698.3.

### Example 91

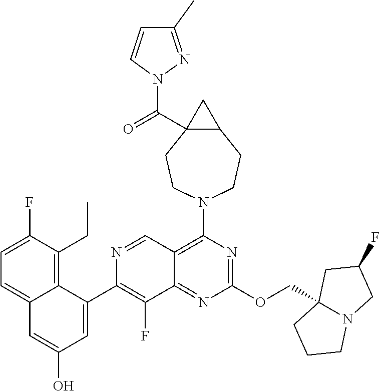 [SMILES: CCc1c(F)ccc2cc(O)cc(-c3ncc4c(N5CCC6CC6(C(=O)n6ccc(C)n6)CC5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[5.1.0]octan-1-yl)(3-methyl-1H-pyrazol-1-yl)methanone

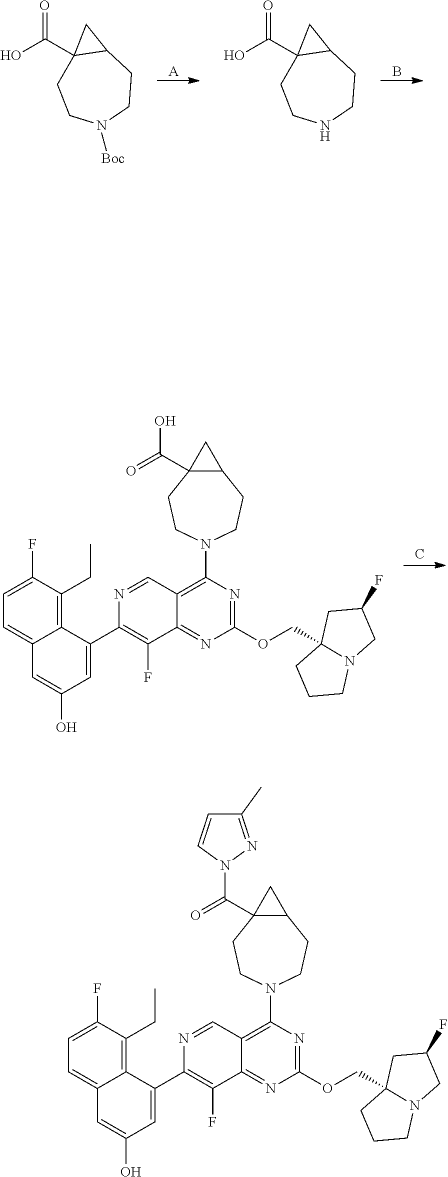

Step A. 4-azabicyclo[5.1.0]octane-1-carboxylic acid: To a solution of 4-(tert-butoxycarbonyl)-4-azabicyclo[5.1.0]octane-1-carboxylic acid (280 mg, 1.0 equiv) in ACN (250 μL) was added HCl·dioxane (2 M, 548 μL, 1.0 equiv). The reaction was stirred at 25 °C for 0.5 hours. The mixture was concentrated under reduced pressure to afford the title compound (170 mg, 90% yield) as white solid.

Step B. 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[5.1.0]octane-1-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and 4-azabicyclo[5.1.0]octane-1-carboxylic acid (170 mg, HCl) in DMF (2.5 mL) were added DIEA (327 mg, 5.0 equiv) and K₃PO₄ (322 mg, 3.0 equiv). The reaction was stirred at 60 °C for 1 hour. The mixture was filtered and purified by reversed phase flash chromatography [C18, 0.1% formic acid condition] to afford the title compound (228 mg, 54% yield, HCOOH salt) as white solid; LCMS (ESI, M+1): m/z=648.3.

Step C. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[5.1.0]octan-1-yl)(3-methyl-1H-pyrazol-1-yl)methanone: To a solution of 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[5.1.0]octane-1-carboxylic acid (50 mg, 1.0 equiv, FA) and 3-methyl-1H-pyrazole (17.8 mg, 3.0 equiv) in DMF (0.5 mL) were added HATU (54.8 mg, 2.0 equiv) and DIEA (55.9 mg, 6.0 equiv). The reaction was stirred at 30 °C for 20 minutes. The mixture was filtered and purified by reversed phase flash chromatography [C18, water/ACN] to afford the title compound (14.6 mg, 28% yield) as yellow solid; ¹H NMR (400 MHz, CHLOROFORM-d) δ=8.95 (s, 1H), 8.13 (br d, J=3.2 Hz, 1H), 7.63-7.52 (m, 1H), 7.25-7.18 (m, 2H), 7.09-7.02 (m, 1H), 6.21 (d, J=2.0 Hz, 1H), 5.40-5.18 (m, 1H), 4.51-4.33 (m, 2H), 4.29-4.18 (m, 2H), 3.97-3.76 (m, 2H), 3.47-3.12 (m, 4H), 3.04-2.95 (m, 1H), 2.80-2.47 (m, 2H), 2.37-2.26 (m, 4H), 2.25-2.09 (m, 3H), 2.04-1.83 (m, 6H), 1.39-1.31 (m, 1H), 1.15-1.08 (m, 1H), 0.92-0.80 (m, 3H); LCMS (ESI, M+1): m/z=712.4.

### Example 92

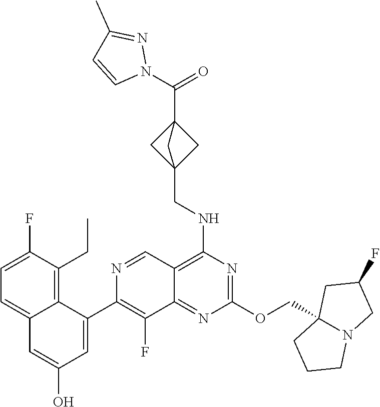 [SMILES: CCc1c(F)ccc2cc(O)cc(-c3ncc4c(NCC56CC(C(=O)n7ccc(C)n7)(C5)C6)nc(OC[C@@]56CCCN5C[C@H](F)C6)nc4c3F)c12]

### (3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)bicyclo[1.1.1]pentan-1-yl)(3-methyl-1H-pyrazol-1-yl)methanone

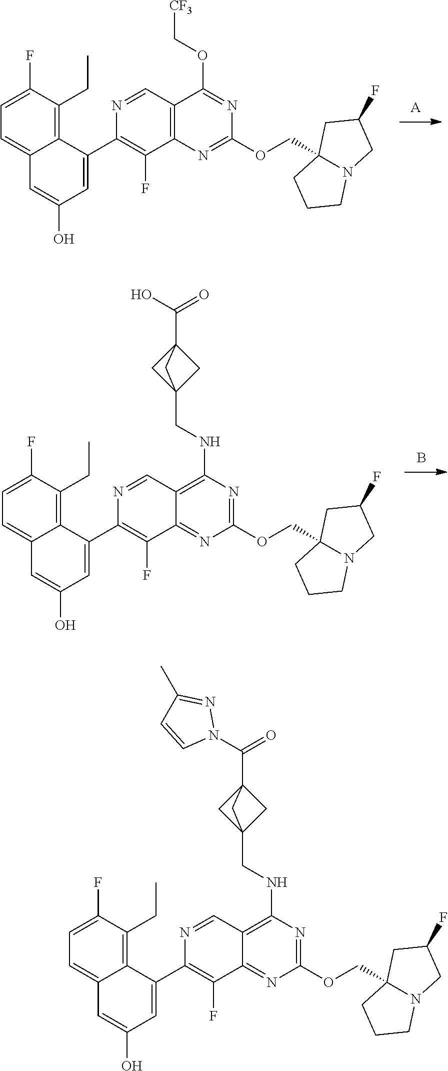

Step A. 3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)bicyclo[1.1.1]pentane-1-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), 3-(aminomethyl)bicyclo[1.1.1]pentane-1-carboxylic acid (108 mg, 1.5 equiv, HCl) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 6 hours. The mixture was filtered and purified by prep-HPLC [column: Phenomenex luna C18 150×40 mm×15 μm; mobile phase: water(FA)-ACN; gradient: 15%-45% B over 15 minutes] to afford the title compound (115 mg, 44% yield) as white solid; LCMS (ESI, M+1): m/z=634.2.

Step B. (3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)bicyclo[1.1.1]pentan-1-yl)(3-methyl-1H-pyrazol-1-yl)methanone: To a solution of 3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)bicyclo[1.1.1]pentane-1-carboxylic acid (110 mg, 1.0 equiv) and 3-methyl-1H-pyrazole (21.8 mg, 1.5 equiv) in DMF (1 mL) were added HATU (99.0 mg, 1.5 equiv) and DIEA (112 mg, 5.0 equiv). The reaction was degassed and purged with nitrogen for 3 times. The reaction was stirred at 25 °C for 1 hour. The mixture was filtered and purified by prep-HPLC [column: Waters Xbridge 150×25 mm×5 μm; mobile phase: water (NH₄HCO₃)-ACN; gradient: 50%-80% B over 9 minutes] to afford the title compound (30.7 mg, 24% yield) as off-white solid; ¹H NMR (400 MHz, CHLOROFORM-d) δ=8.99-8.75 (m, 1H), 8.08 (t, J=3.2 Hz, 1H), 7.54-7.39 (m, 1H), 7.17-7.04 (m, 2H), 6.93-6.78 (m, 1H), 6.20 (br s, 1H), 5.40-5.13 (m, 1H), 4.38-4.19 (m, 2H), 3.86-3.48 (m, 2H), 3.36-3.14 (m, 3H), 3.05-2.94 (m, 1H), 2.54-2.40 (m, 1H), 2.30-2.25 (m, 9H), 2.09 (br d, J=10.0 Hz, 4H), 1.99-1.90 (m, 3H), 0.81-0.72 (m, 3H); LCMS (ESI, M+1): m/z=698.3.

### Example 93

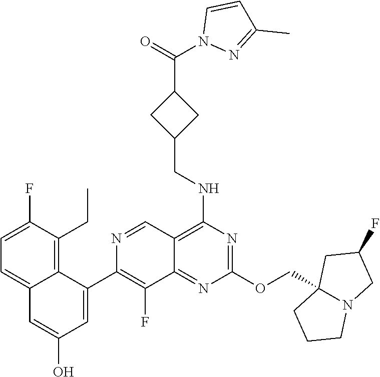 [SMILES: CCc1c(F)ccc2cc(O)cc(-c3ncc4c(NCC5CC(C(=O)n6ccc(C)n6)C5)nc(OC[C@@]56CCCN5C[C@H](F)C6)nc4c3F)c12]

### (3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclobutyl)(3-methyl-1H-pyrazol-1-yl)methanone

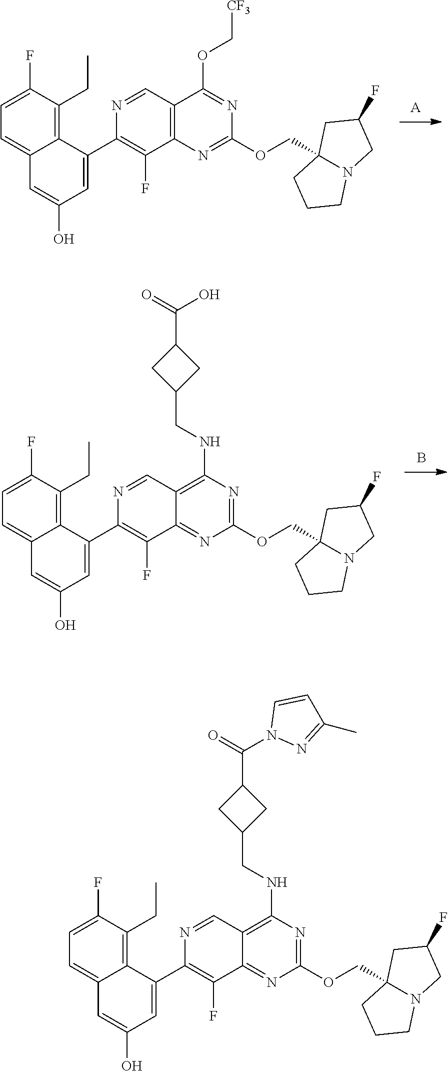

Step A. 3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclobutane-1-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and 3-(aminomethyl)cyclobutane-1-carboxylic acid (251 mg, 3.0 equiv, HCl) in DMF (2.5 mL) were added DIEA (523 mg, 8.0 equiv) and K₃PO₄ (322 mg, 3.0 equiv). The reaction was stirred at 60 °C for 1.5 hours. The mixture was filtered and purified by reversed phase flash chromatography [C18, 0.1% formic acid condition] to afford the title compound (218 mg, 68% yield, HCOOH salt) as white solid; LCMS (ESI, M+1): m/z=622.3.

Step B. (3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclobutyl)(3-methyl-1H-pyrazol-1-yl)methanone: To a solution of 3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclobutane-1-carboxylic acid (70 mg, 1.0 equiv, FA) and 3-methyl-1H-pyrazole (43.0 mg, 5.0 equiv) in DMF (0.7 mL) were added HATU (79.7 mg, 2.0 equiv) and DIEA (67.7 mg, 5.0 equiv). The reaction was stirred at 30 °C for 15 minutes. The mixture was filtered and purified by reversed phase flash chromatography [C18, water/ACN] to afford the title compound (11.8 mg, 16% yield) as white solid; ¹H NMR (400 MHz, CHLOROFORM-d) δ=9.17-8.79 (m, 1H), 8.19-8.10 (m, 1H), 7.62-7.46 (m, 1H), 7.22-7.09 (m, 2H), 7.00-6.78 (m, 1H), 6.25 (d, J=2.8 Hz, 1H), 5.45-5.14 (m, 1H), 4.39-4.18 (m, 3H), 3.84-3.52 (m, 2H), 3.41-3.11 (m, 3H), 3.05-2.96 (m, 1H), 2.87-2.66 (m, 1H), 2.62-2.40 (m, 3H), 2.32 (s, 3H), 2.29-2.21 (m, 2H), 2.20-2.08 (m, 3H), 2.06-1.91 (m, 4H), 0.86-0.72 (m, 3H); LCMS (ESI, M+1): m/z=686.4.

### Example 94

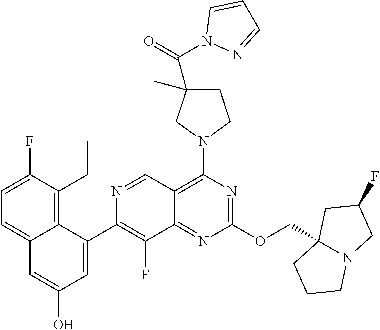 [SMILES: CCc1c(F)ccc2cc(O)cc(-c3ncc4c(N5CCC(C)(C(=O)n6cccn6)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpyrrolidin-3-yl)(3-methyl-1H-pyrazol-1-yl)methanone

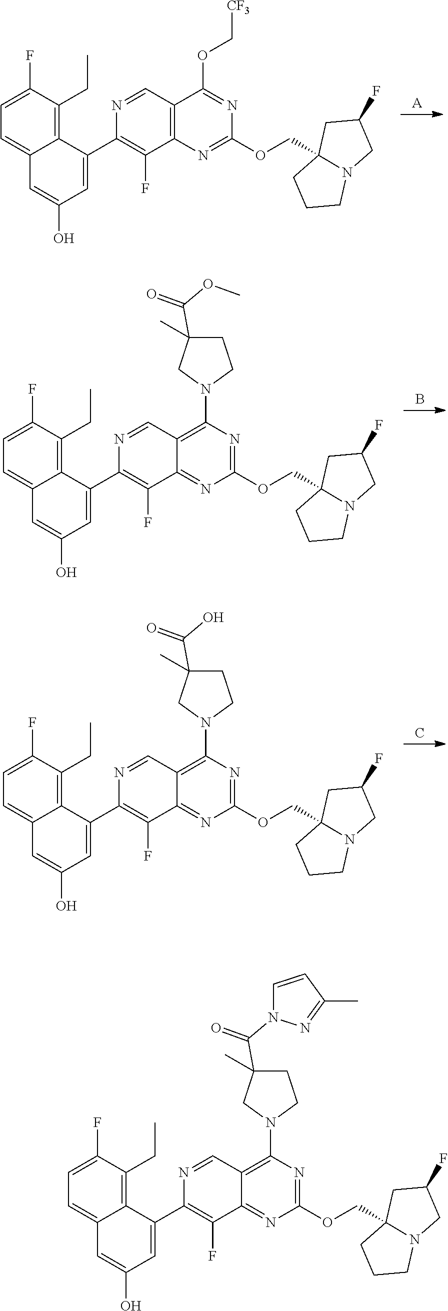

Step A. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpyrrolidine-3-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and methyl 3-methylpyrrolidine-3-carboxylate (217 mg, 3 equiv) in DMF (3 mL) were added DIEA (327 mg, 5.0 equiv). The reaction was stirred at 30 °C for 0.5 hours. The mixture was quenched with H₂O (5 mL) and extracted with ethyl acetate mL (15 mL×3). The combined organic layers were washed with brine (4 mL×2), dried over anhydrous sodium sulfate and concentrated to afford the title compound (300 mg, 91% yield) as orange oil; LCMS (ESI, M+1): m/z=636.2.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpyrrolidine-3-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpyrrolidine-3-carboxylate (280 mg, 1.0 equiv) in MeOH (0.8 mL) were added LiOH·H₂O (2 M, 0.8 mL, 4.0 equiv). The reaction was stirred at 25 °C for 20 minutes. The mixture was filtered and purified by reversed phase flash chromatography [C18, 0.1% formic acid condition] to afford the title compound (270 mg, 96% yield, HCOOH salt) as white solid; LCMS (ESI, M+1): m/z=622.3

Step C. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpyrrolidin-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpyrrolidine-3-carboxylic acid (100 mg, 1.0 equiv, FA) and 3-methyl-1H-pyrazole (61 mg, 5.0 equiv) in DMF (1 mL) were added HATU (114 mg, 2.0 equiv) and DIEA (116 mg, 6.0 equiv). The reaction was stirred at 30 °C for 15 minutes. The mixture was filtered and purified by reversed phase flash chromatography [C18, neutral condition] to afford the title compound (7.62 mg, 7% yield) as white solid; ¹H NMR (400 MHz, CHLOROFORM-d) δ=9.21-9.14 (m, 1H), 8.17 (br s, 1H), 7.58-7.49 (m, 1H), 7.22-7.12 (m, 2H), 7.04-6.93 (m, 1H), 6.26 (d, J=2.8 Hz, 1H), 5.41-5.18 (m, 1H), 4.69-4.20 (m, 4H), 4.13-3.99 (m, 2H), 3.37-3.15 (m, 3H), 3.06-2.95 (m, 1H), 2.89-2.68 (m, 1H), 2.52-2.40 (m, 1H), 2.37-2.30 (m, 3H), 2.26-2.09 (m, 4H), 2.04-1.91 (m, 4H), 1.73 (br d, J=2.4 Hz, 3H), 0.92-0.76 (m, 3H); LCMS (ESI, M+1): m/z=686.3.

### Example 95

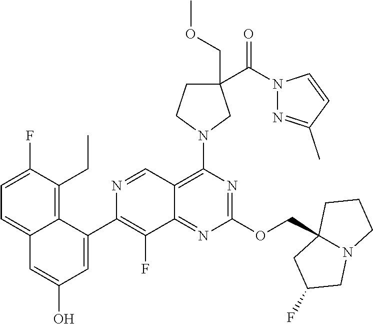 [SMILES: CCc1c(F)ccc2cc(O)cc(-c3ncc4c(N5CCC(COC)(C(=O)n6ccc(C)n6)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(methoxymethyl) pyrrolidin-3-yl)(3-methyl-1H-pyrazol-1-yl)methanone

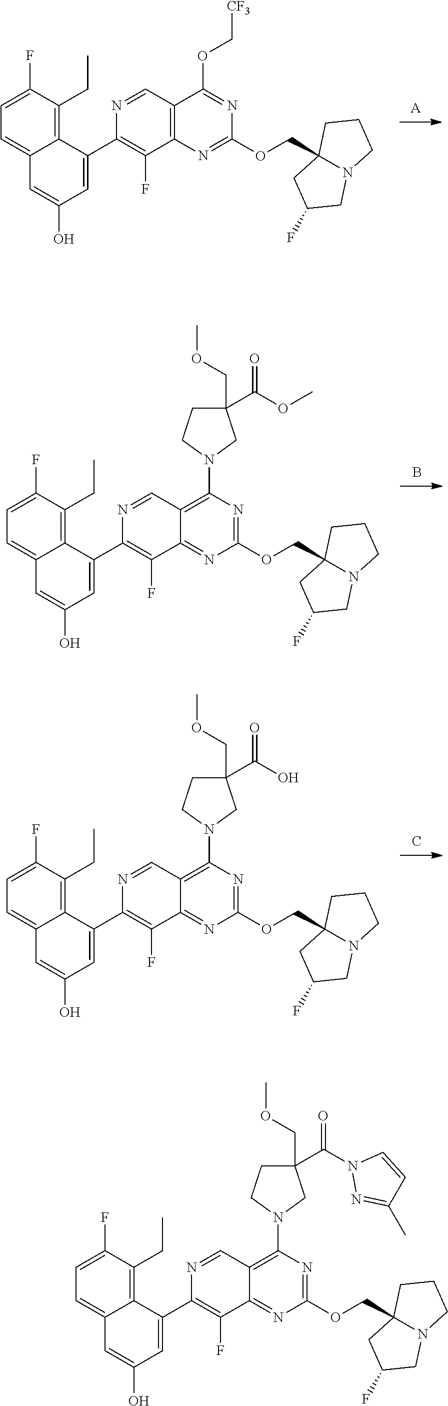

Step A. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(methoxymethyl) pyrrolidine-3-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and methyl 3-(methoxymethyl) pyrrolidine-3-carboxylate (263 mg, 3.0 equiv) in DMF (3 mL) was added DIEA (327 mg, 5.0 equiv). The reaction was stirred at 30 °C for 0.5 hours. The mixture was quenched with H₂O (5 mL) and extracted with ethyl acetate (3×15 mL). The combined organic layers were washed with brine (2×4 mL), dried over anhydrous sodium sulfate and concentrated to afford the title compound (300 mg, 87% yield) as orange oil; LCMS (ESI, M+1): m/z=666.3.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(methoxymethyl) pyrrolidine-3-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(methoxymethyl) pyrrolidine-3-carboxylate (280 mg, 1.0 equiv) in MeOH (0.8 mL) was added LiOH·H₂O (2 M, 0.8 ml, 4.0 equiv). The reaction was stirred at 25 °C for 20 minutes. The mixture was filtered and purified by reversed phase flash chromatography [C18, 0.1% formic acid condition] to afford the title compound (270 mg, 98% yield, HCOOH salt) as white solid; LCMS (ESI, M+1): m/z=652.3.

Step C. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(methoxymethyl) pyrrolidin-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(methoxymethyl) pyrrolidine-3-carboxylic acid (100 mg, 1.0 equiv) and 3-methyl-1H-pyrazole (63.0 mg, 5.0 equiv) in DMF (1 mL) were added HATU (117 mg, 2.0 equiv) and DIEA (99.2 mg, 5.0 equiv). The reaction was stirred at 30 °C for 10 minutes. The mixture was filtered and purified by reversed phase flash chromatography [C18, neutral condition] to afford the title compound (34.1 mg, 31% yield) as white solid; ¹H NMR (400 MHz, CHLOROFORM-d) 8=9.21-9.10 (m, 1H), 8.18 (dd, J=2.4, 6.4 Hz, 1H), 7.50 (ddd, J=2.8, 5.6, 8.8 Hz, 1H), 7.19-7.08 (m, 2H), 6.99-6.90 (m, 1H), 6.26 (t, J=2.8 Hz, 1H), 5.40-5.19 (m, 1H), 4.70-4.20 (m, 4H), 4.10-3.93 (m, 4H), 3.41-3.10 (m, 6H), 3.06-2.96 (m, 1H), 2.88-2.64 (m, 1H), 2.54-2.41 (m, 2H), 2.33 (br d, J=7.6 Hz, 3H), 2.29-2.09 (m, 5H), 1.97 (br dd, J=4, 10.8 Hz, 2H), 0.87-0.76 (m, 3H); LCMS (ESI, M+1): m/z=716.4.

### Example 96

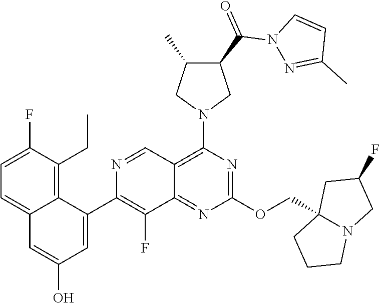 [SMILES: CCc1c(F)ccc2cc(O)cc(-c3ncc4c(N5C[C@H](C(=O)n6ccc(C)n6)[C@@H](C)C5)nc(OC[C@@]56CCCN5C[C@H](F)C6)nc4c3F)c12]

### ((3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpyrrolidin-3-yl)(3-methyl-1H-pyrazol-1-yl)methanone

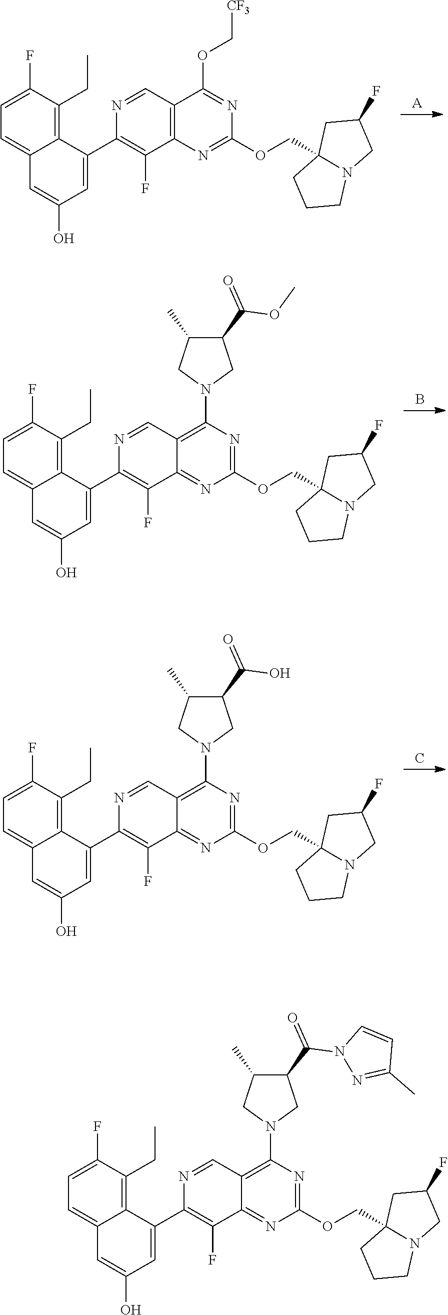

Step A. methyl (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpyrrolidine-3-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (200 mg, 1.0 equiv) and methyl (3R,4R)-4-methylpyrrolidine-3-carboxylate (78.8 mg, 1.3 equiv, HCl) in DMAC (1 mL) was added DIEA (218 mg, 5.0 equiv). The reaction was stirred at 30 °C for 2 hours. The mixture was filtered and purified by reversed phase flash [C18, 0.1% formic acid condition] to afford the title compound (125 mg, 56% yield) as white solid; LCMS (ESI, M+1): m/z=636.4.

Step B. (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpyrrolidine-3-carboxylic acid: To a solution of methyl (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpyrrolidine-3-carboxylate (120 mg, 1.0 equiv) in MeOH (5 mL) was added LiOH·H₂O (2 M in water, 0.47 mL, 5.0 equiv). The reaction was stirred at 25 °C for 1 hour. The mixture was concentrated and diluted with water (3 mL). The mixture was adjusted to pH=7 with 0.1 M HCl and filtered to give a residue. The residue was added to toluene (10 mL) and concentrated to afford the title compound (115 mg, 98% yield) as white solid; LCMS (ESI, M+1): m/z=622.3.

Step C. ((3R,4R)-1-(7-(8-ethyl-7-fluoro-3-hydroxynaphthalen-1-yl)-8-fluoro-2-yl)-4-methylpyrrolidin-3-yl)(3-methyl-1H-pyrazol-1-yl)methanone: To a solution of (3R,4R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methylpyrrolidine-3-carboxylic acid (110 mg, 1.0 equiv), 3-methyl-1H-pyrazole (21.8 mg, 1.5 equiv) and DIEA (68.6 mg, 3.0 equiv) in DMF (1 mL) was added HATU (101 mg, 1.5 equiv). The reaction was stirred at 25 °C for 1 hour. The mixture was filtered and purified by prep-HPLC [column: Waters Xbridge 150×25 mm×5 μm; A: water (NH₄HCO₃), B: ACN, B %: 48%-68% over 10 min] and prep-HPLC [column: Waters Xbridge 150×25 mm×5 μm; A: water (NH₄HCO₃), B: ACN, B %: 48%-68% over 10 min] to afford the title compound (14.9 mg, 12% yield) as off-white solid; ¹H NMR (400 MHz, DMSO-d₆) δ=9.93 (br s, 1H), 9.27 (br d, J=6.8 Hz, 1H), 8.37 (br s, 1H), 7.76 (br t, J=6.8 Hz, 1H), 7.44-7.24 (m, 2H), 7.01 (br d, J=8.0 Hz, 1H), 6.53 (br s, 1H), 5.43-5.12 (m, 1H), 4.52-4.05 (m, 5H), 3.15-2.97 (m, 3H), 2.81 (br d, J=7.2 Hz, 2H), 2.56 (br s, 2H), 2.30 (br s, 5H), 2.18-2.02 (m, 3H), 1.92-1.72 (m, 3H), 1.21-1.14 (m, 3H), 0.82-0.62 (m, 3H); LCMS (ESI, M+1): m/z=686.4.

### Example 97

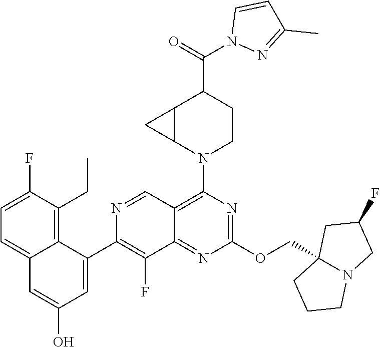 [SMILES: CCc1c(F)ccc2cc(O)cc(-c3ncc4c(N5CCC(C(=O)n6ccc(C)n6)C6CC65)nc(OC[C@@]56CCCN5C[C@H](F)C6)nc4c3F)c12]

### (2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[4.1.0]heptan-5-yl)(3-methyl-1H-pyrazol-1-yl)methanone

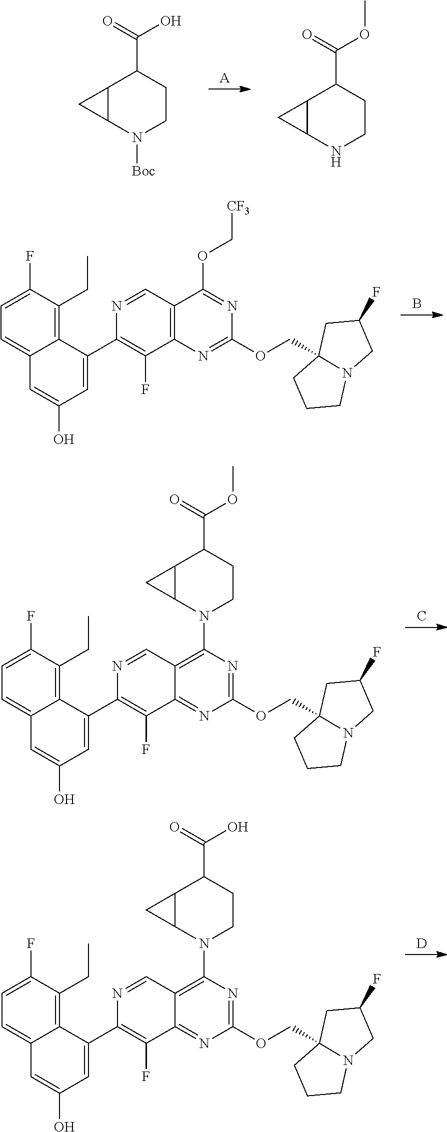

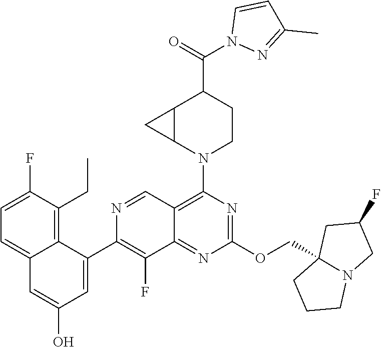 [SMILES: CCc1c(F)ccc2cc(O)cc(-c3ncc4c(N5CCC(C(=O)n6ccc(C)n6)C6CC65)nc(OC[C@@]56CCCN5C[C@H](F)C6)nc4c3F)c12]

Step A. methyl 2-azabicyclo[4.1.0]heptane-5-carboxylate: To a solution of 2-(tert-butoxycarbonyl)-2-azabicyclo[4.1.0]heptane-5-carboxylic acid (120 mg, 1.0 equiv) in MeOH (0.5 mL) was added HCl·MeOH (2 M, 2 mL, 8.0 equiv). The reaction was stirred at 0 °C for 1 hour. The mixture was concentrated to afford the title compound (80.0 mg, crude) as yellow oil; ¹H NMR (400 MHz, CHLOROFORM-d) δ=3.77 (d, J=12.0 Hz, 3H), 3.50 (s, 1H), 3.35-2.75 (m, 4H), 2.14-1.97 (m, 1H), 1.90-1.78 (m, 1H), 1.75-1.51 (m, 1H), 1.33-1.04 (m, 2H).

Step B. methyl 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[4.1.0]heptane-5-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (160 mg, 1.0 equiv) and methyl 2-azabicyclo[4.1.0]heptane-5-carboxylate (55.0 mg, crude) in DMAC (1 mL) was added K₃PO₄ (174 mg, 3.0 equiv). The reaction was stirred at 40 °C for 6 hours. The mixture was filtered and purified by reversed phase flash [C18, 0.1% formic acid condition] to afford the title compound (110 mg, 56% yield) as yellow solid; LCMS (ESI, M+1): m/z=648.4.

Step C. 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[4.1.0]heptane-5-carboxylic acid: To a solution of methyl 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[4.1.0]heptane-5-carboxylate (105 mg, 1.0 equiv) in MeOH (2 mL) was added LiOH·H₂O (2 M, 0.4 mL, 5.0 equiv). The reaction was stirred at 25 °C for 1 hour. The mixture was concentrated and diluted with water (3 mL). The mixture was adjusted to pH=7 with 0.1 M HCl and filtered to give a residue. The residue was added toluene (10 mL) and concentrated to afford the title compound (100 mg, 97% yield) as white solid; LCMS (ESI, M+1): m/z=634.3.

Step D. (2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[4.1.0]heptan-5-yl)(3-methyl-1H-pyrazol-1-yl)methanone: To a solution of 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-azabicyclo[4.1.0]heptane-5-carboxylic acid (95.0 mg, 1.0 equiv), 3-methyl-1H-pyrazole (18.5 mg, 1.5 equiv) and DIEA (58.1 mg, 3.0 equiv) in DMF (1 mL) was added HATU (85.5 mg, 1.5 equiv). The reaction was stirred at 25 °C for 1 hour. The mixture was filtered and purified by prep-HPLC [column: Waters Xbridge 150×25 mm×5 μm; A: water (NH₄HCO₃), B: ACN, B %: 47%-67% over 10 minutes] to afford the title compound (23.6 mg, 22% yield) as white solid; ¹H NMR (400 MHz, DMSO-d₆) δ=9.93 (br s, 1H), 9.85-9.68 (m, 1H), 8.43-8.29 (m, 1H), 7.85-7.70 (m, 1H), 7.41-7.27 (m, 2H), 7.10-6.95 (m, 1H), 6.50 (d, J=2.4 Hz, 1H), 5.40-5.15 (m, 1H), 4.86-4.40 (m, 1H), 4.36-4.03 (m, 3H), 3.74-3.53 (m, 1H), 3.29-3.18 (m, 1H), 3.12-3.00 (m, 3H), 2.89-2.76 (m, 1H), 2.43-2.26 (m, 4H), 2.22-2.00 (m, 6H), 1.92-1.69 (m, 4H), 1.36-1.20 (m, 1H), 0.98-0.83 (m, 1H), 0.74 (q, J=7.6 Hz, 3H); LCMS (ESI, M+1): m/z=698.4.

### Example 98

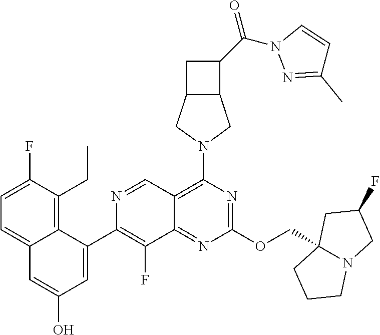 [SMILES: CCc1c(F)ccc2cc(O)cc(-c3ncc4c(N5CC6CC(C(=O)n7ccc(C)n7)C6C5)nc(OC[C@@]56CCCN5C[C@H](F)C6)nc4c3F)c12]

### (3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone

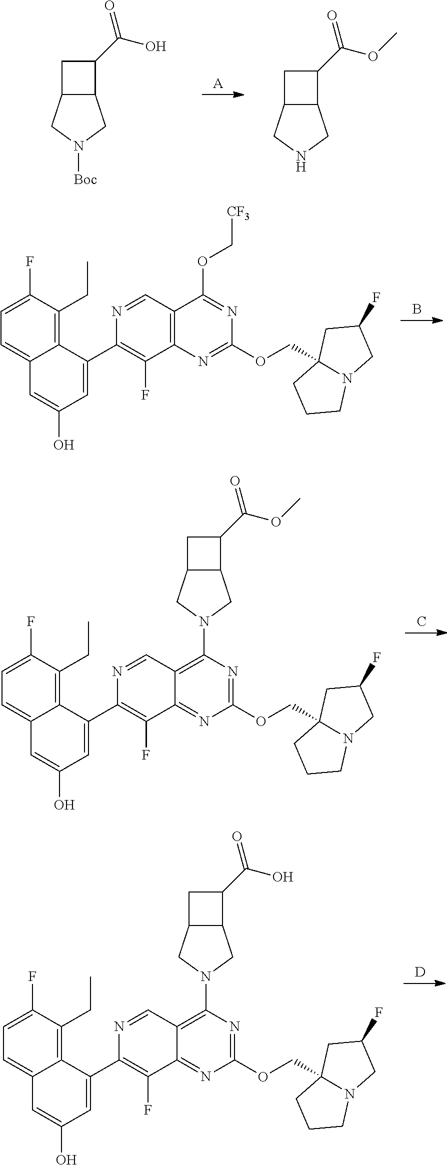

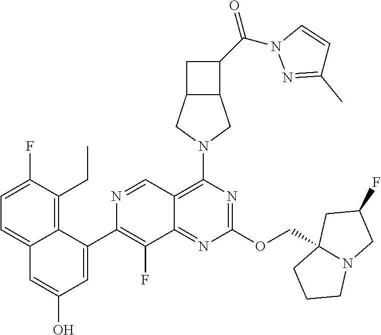 [SMILES: CCc1c(F)ccc2cc(O)cc(-c3ncc4c(N5CC6CC(C(=O)n7ccc(C)n7)C6C5)nc(OC[C@@]56CCCN5C[C@H](F)C6)nc4c3F)c12]

Step A. methyl 3-azabicyclo[3.2.0]heptane-6-carboxylate: To a solution of 3-(tert-butoxycarbonyl)-3-azabicyclo[3.2.0]heptane-6-carboxylic acid (150 mg, 1.0 equiv) in MeOH (1 mL) was added HCl·MeOH (2 M, 2.5 mL, 8.0 equiv). The reaction was stirred at 0 °C for 1 hour. The mixture was concentrated to afford the title compound (130 mg, crude) as yellow oil.

Step B. methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (200 mg, 1.0 equiv) and methyl 3-azabicyclo[3.2.0]heptane-6-carboxylate (130 mg, crude, HCl) in DMAC (1 mL) was added K₃PO₄ (358 mg, 5.0 equiv). The reaction was stirred at 40 °C for 3 hours. The mixture was filtered and purified by reversed phase flash [C18, 0.1% formic acid] to afford the title compound (145 mg, 25% yield) as yellow solid; LCMS (ESI, M+1): m/z=648.3.

Step C. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid: To a solution of methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate (140 mg, 1.0 equiv) in MeOH (2 mL) was added LiOH·H₂O (2 M in water, 0.54 mL, 5.0 equiv). The reaction was stirred at 25 °C for 1 hour. The mixture was concentrated and diluted with water (3 mL). The mixture was adjusted to pH=7 with 0.1 M HCl and filtered to give a residue. Toluene (10 mL) was added to the residue and concentrated to afford the title compound (135 mg, 98% yield) as white solid; LCMS (ESI, M+1): m/z=634.3.

Step D. (3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone: To a solution of 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (130 mg, 1.0 equiv), 3-methyl-1H-pyrazole (25.3 mg, 1.5 equiv) and DIEA (79.5 mg, 3.0 equiv) in DMF (1 mL) was added HATU (117 mg, 1.5 equiv). The reaction was stirred at 25 °C for 1 hour. The mixture was filtered and purified by prep-HPLC [column: Waters Xbridge 150×25 mm×5 μm; A: water (NH₄HCO₃), B: ACN, B %: 44%-64% over 10 minutes] to afford the title compound (36.6 mg, 25% yield) as yellow solid; ¹H NMR (400 MHz, DMSO-d₆) δ=9.93 (br s, 1H), 9.18 (br s, 1H), 8.30-8.13 (m, 1H), 7.76 (br dd, J=6.0, 8.8 Hz, 1H), 7.43-7.27 (m, 2H), 7.01 (br s, 1H), 6.45 (br s, 1H), 5.43-5.13 (m, 1H), 4.41-4.20 (m, 2H), 4.14-4.06 (m, 1H), 4.01-3.79 (m, 3H), 3.58 (br d, J=7.6 Hz, 1H), 3.25 (br s, 1H), 3.14-2.95 (m, 3H), 2.88-2.75 (m, 1H), 2.48-2.21 (m, 7H), 2.17-2.00 (m, 3H), 1.98-1.65 (m, 4H), 0.85-0.61 (m, 3H); LCMS (ESI, M+1): m/z=698.4.

### Example 99

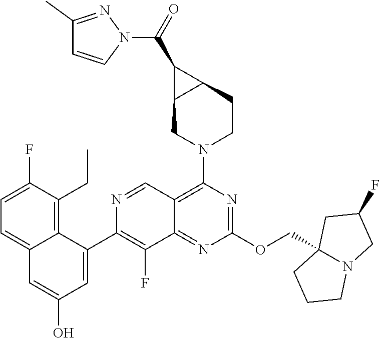 [SMILES: CCc1c(F)ccc2cc(O)cc(-c3ncc4c(N5CC[C@H]6[C@@H](C5)[C@H]6C(=O)n5ccc(C)n5)nc(OC[C@@]56CCCN5C[C@H](F)C6)nc4c3F)c12]

### (cis-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptan-7-yl)(3-methyl-1H-pyrazol-1-yl)methanone

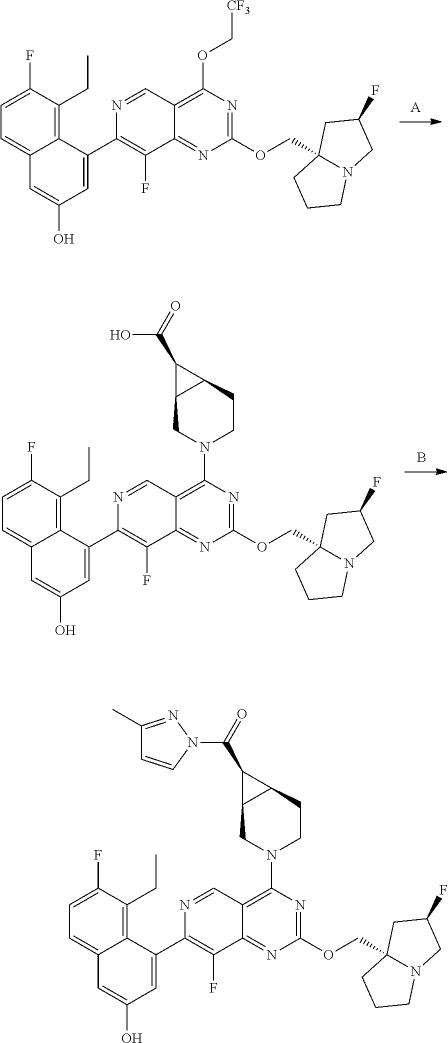

Step A. (1S,6R,7R)-3-(7-(8-ethyl-7-fluoro-3-hydroxynaphthalen-1-yl)-8-fluoro-2-(((2S,7aR)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid: To a solution of rac-(1R,6S,7S)-3-azabicyclo[4.1.0]heptane-7-carboxylic acid (180 mg, 2.0 equiv, HCl) in DMF (3 mL) were added K₃PO₄ (322 mg, 3.0 equiv) and 4 Å molecular sieve (150 mg) slowly at 25 °C. The reaction was stirred at 25 °C for 30 minutes. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) was added at 25 °C slowly. The reaction was stirred at 60 °C for 4 hours. The mixture was filtered and purified by reversed phase flash [C18, 0.1% formic acid condition] to afford the title compound (150 mg, 37% yield, HCOOH salt) as white solid; LCMS (ESI, M+1): m/z=634.4.

Step B. ((1R,6S,7S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptan-7-yl)(3-methyl-1H-pyrazol-1-yl)methanone: To a solution of (1S,6R,7R)-3-(7-(8-ethyl-7-fluoro-3-hydroxynaphthalen-1-yl)-8-fluoro-2-(((2S,7aR)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid (10.0 mg, 1.0 equiv, HCOOH) and 3-methyl-1H-pyrazole (3.62 mg, 3.0 equiv) in DMF (0.5 mL) were added HATU (16.8 mg, 3.0 equiv) and DIEA (11.4 mg, 6.0 equiv). The reaction was stirred at 25 °C for 1 hour. The mixture was filtered and purified by reversed phase flash chromatography [Waters Xbridge 150×25 mm×5 μm; A: water (NH₄HCO₃), B: ACN, B %: 50%-80% over 9 minutes] to afford the title compound (4.10 mg, 37% yield) as yellow solid; ¹H NMR (400 MHz, DMSO-d6) δ=10.02-9.83 (m, 1H), 9.18-9.03 (m, 1H), 8.28 (d, J=2.8 Hz, 1H), 7.76 (dd, J=6.0, 9.2 Hz, 1H), 7.39-7.31 (m, 2H), 7.02 (dd, J=2.4, 8.4 Hz, 1H), 6.49-6.44 (m, 1H), 5.38-5.15 (m, 1H), 4.48-4.31 (m, 1H), 4.29-4.21 (m, 1H), 4.16 (br dd, J=2.4, 11.2 Hz, 1H), 4.10-4.04 (m, 1H), 4.01-3.91 (m, 1H), 3.75-3.61 (m, 1H), 3.16-3.05 (m, 3H), 2.88-2.78 (m, 1H), 2.42-2.31 (m, 3H), 2.24 (d, J=5.2 Hz, 3H), 2.19-2.00 (m, 7H), 1.90-1.78 (m, 2H), 0.95 (d, J=6.4 Hz, 1H), 0.72 (td, J=7.6, 14.8 Hz, 3H); LCMS (ESI, M+1): m/z=698.4.

### Example 100

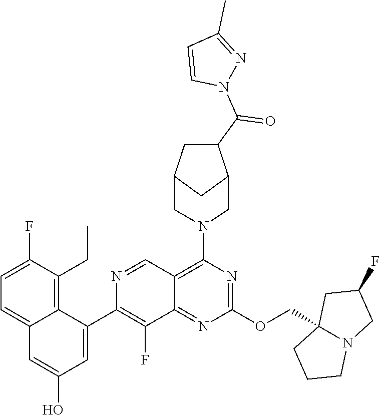 [SMILES: CCc1c(F)ccc2cc(O)cc(-c3ncc4c(N5CC6CC(C5)C(C(=O)n5ccc(C)n5)C6)nc(OC[C@@]56CCCN5C[C@H](F)C6)nc4c3F)c12]

### ((1S,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-yl)(3-methyl-1H-pyrazol-1-yl)methanone

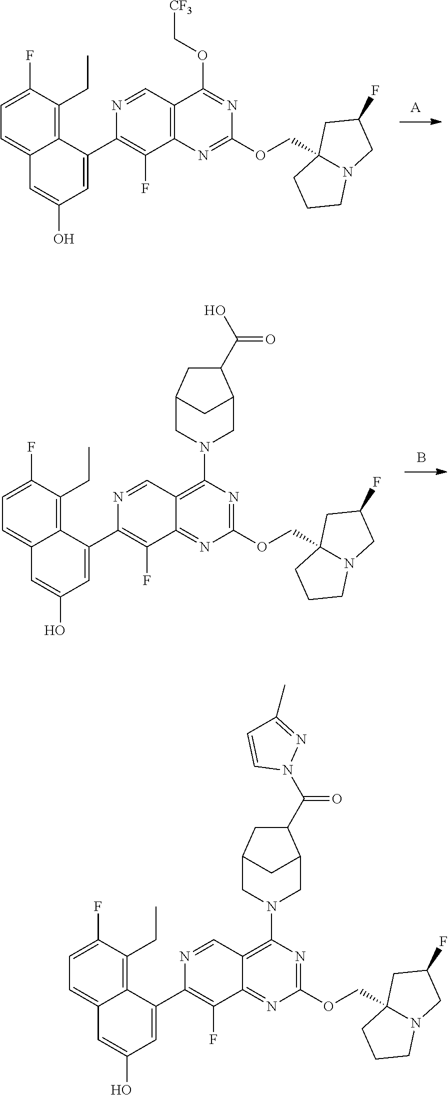

Step A. (1S,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-6-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), (1S,5R,6R)-3-azabicyclo[3.2.1]octane-6-carboxylic acid (116 mg, 1.5 equiv, HCl) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 6 hours. The mixture was filtered and purified by prep-HPLC [column: Phenomenex luna C18 150×40 mm×15 μm; mobile phase: water(FA)-ACN; gradient: 15%-45% B over 15 minutes] to afford the title compound (100 mg, 38% yield) as white solid; LCMS (ESI, M+1): m/z=648.3.

Step B. ((1S,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-yl)(3-methyl-1H-pyrazol-1-yl)methanone: To a solution of (1S,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-6-carboxylic acid (95.0 mg, 1.0 equiv) and 3-methyl-1H-pyrazole (18.1 mg, 1.5 equiv) in DMF (1 mL) were added HATU (83.7 mg, 1.5 equiv) and DIEA (94.8 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 48%-78% B over 20 minutes] to afford the title compound (46.2 mg, 44% yield) as yellow solid; ¹H NMR (400 MHz, CHLOROFORM-d) δ=9.04-8.90 (m, 1H), 7.98-7.85 (m, 1H), 7.51 (dd, J=6.0, 9.2 Hz, 1H), 7.21-7.10 (m, 2H), 7.07-6.96 (m, 1H), 6.22-6.14 (m, 1H), 5.36-5.15 (m, 1H), 5.14-4.66 (m, 1H), 4.60-4.16 (m, 2H), 4.15-3.94 (m, 2H), 3.76-3.28 (m, 2H), 3.23-2.93 (m, 4H), 2.66-2.52 (m, 1H), 2.50-2.39 (m, 1H), 2.37-2.24 (m, 5H), 2.23-2.04 (m, 5H), 2.00-1.77 (m, 5H), 0.89-0.79 (m, 3H); LCMS (ESI, M+1): m/z=712.3.

### Example 101

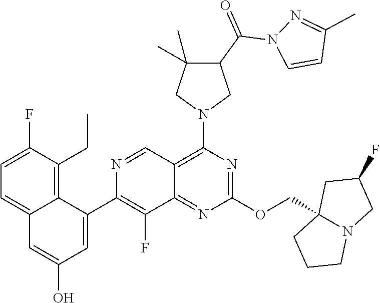 [SMILES: CCc1c(F)ccc2cc(O)cc(-c3ncc4c(N5CC(C(=O)n6ccc(C)n6)C(C)(C)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,4-dimethylpyrrolidin-3-yl)(3-methyl-1H-pyrazol-1-yl)methanone

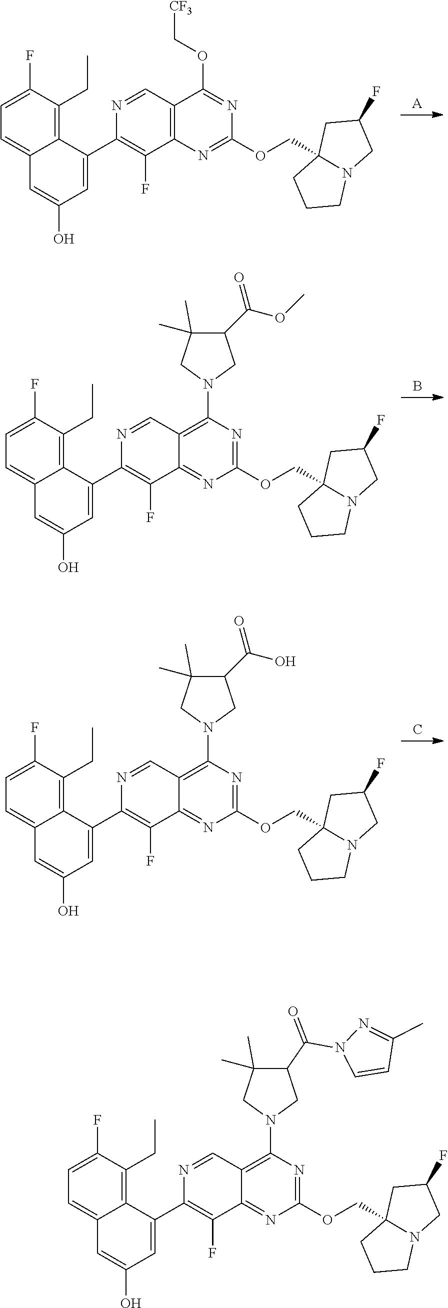

Step A. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,4-dimethylpyrrolidine-3-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), methyl 4,4-dimethylpyrrolidine-3-carboxylate (118 mg, 1.5 equiv, HCl) and 4 Å molecular sieve (50 mg) in DMF (4 mL) was added K₃PO₄ (258 mg, 3.0 equiv). The reaction was degassed and purged with nitrogen for 3 times. The reaction was stirred at 60 °C for 1 hour. The mixture was quenched with water (20 mL) and extracted with ethyl acetate (20 mL×3). The combined organic layers were washed with brine (20 mL×2), dried over anhydrous sodium sulfate and concentrated under reduced pressure to afford the title compound (280 mg, crude) as yellow oil; LCMS (ESI, M+1): m/z=650.3.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,4-dimethylpyrrolidine-3-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,4-dimethylpyrrolidine-3-carboxylate (260 mg, 1.0 equiv) in DMSO (3 mL) was added LiOH·H₂O (2 M, 0.6 ml, 3.0 equiv). The reaction was degassed and purged with nitrogen 3 times. The reaction was stirred at 25 °C for 2 hours. The mixture was filtered and purified by prep-HPLC [column: Phenomenex luna C18 150×40 mm×15 μm; mobile phase: water(FA)-ACN; gradient: 18%-48% B over 15 minutes] to afford the title compound (110 mg, 43% yield) as white solid; LCMS (ESI, M+1): m/z=636.3.

Step C. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,4-dimethylpyrrolidin-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,4-dimethylpyrrolidine-3-carboxylic acid (105 mg, 1.0 equiv) and 3-methyl-1H-pyrazole (20.3 mg, 1.5 equiv) in DMF (1 mL) were added HATU (94.2 mg, 1.5 equiv) and DIEA (107 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 50%-80% B over 20 minutes] to afford the title compound (20.8 mg, 18% yield) as off-white solid; ¹H NMR (400 MHz, CHLOROFORM-d) δ=9.17 (br d, J=3.2 Hz, 1H), 8.18 (d, J=2.4 Hz, 1H), 7.52 (dd, J=6.0, 8.8 Hz, 1H), 7.21-7.10 (m, 2H), 7.05-6.94 (m, 1H), 6.33 (d, J=2.8 Hz, 1H), 5.39-5.15 (m, 1H), 4.53-4.43 (m, 1H), 4.40-4.18 (m, 4H), 4.12-3.93 (m, 1H), 3.92-3.81 (m, 1H), 3.33-3.11 (m, 3H), 3.02-2.91 (m, 1H), 2.49 (br dd, J=6.8, 13.2 Hz, 1H), 2.36 (s, 3H), 2.30-2.20 (m, 2H), 2.18-2.07 (m, 2H), 1.92 (m, 3H), 1.34 (s, 3H), 1.14 (s, 3H), 0.83 (br t, J=7.2 Hz, 3H); LCMS (ESI, M+1): m/z=700.3.

### Example 102

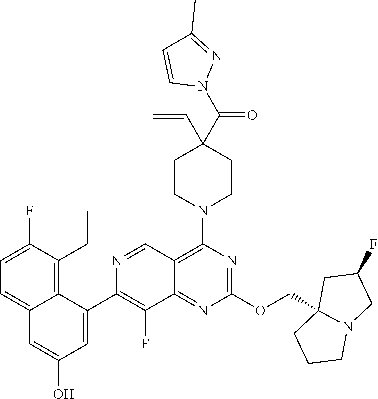 [SMILES: C=CC1(C(=O)n2ccc(C)n2)CCN(c2nc(OC[C@@]34CCCN3C[C@H](F)C4)nc3c(F)c(-c4cc(O)cc5ccc(F)c(CC)c45)ncc23)CC1]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-vinylpiperidin-4-yl)(3-methyl-1H-pyrazol-1-yl)methanone

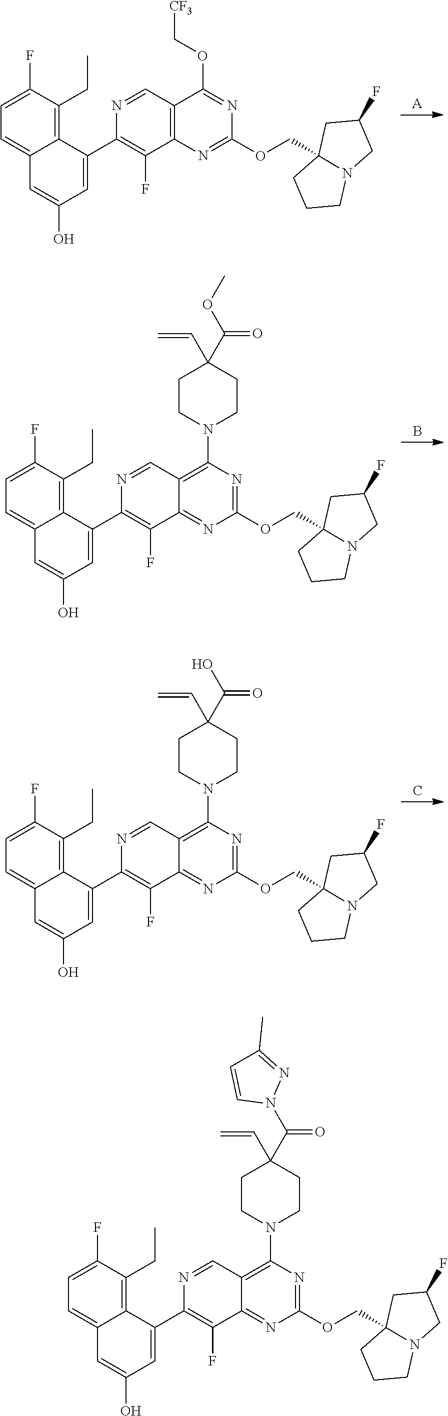

Step A. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-vinylpiperidine-4-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), methyl 4-vinylpiperidine-4-carboxylate (125 mg, 1.5 equiv, HCl) and 4 Å molecular sieve (50 mg) in DMF (4 mL) was added K₃PO₄ (258 mg, 3.0 equiv). The reaction was degassed and purged with nitrogen for 3 times. The reaction was stirred at 60 °C for 1 hour. The mixture was quenched with water (20 mL) and extracted with ethyl acetate (20 mL×3). The combined organic layers were washed with brine (20 mL×2), dried over anhydrous sodium sulfate and concentrated under reduced pressure to afford the title compound (280 mg, crude) as yellow oil; LCMS (ESI, M+1): m/z=662.2.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-vinylpiperidine-4-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-vinylpiperidine-4-carboxylate (260 mg, 1.0 equiv) in DMSO (3 mL) was added LiOH·H₂O (2 M, 1.0 ml, 5.0 equiv). The reaction was degassed and purged with nitrogen for 3 times. The reaction was stirred at 25 °C for 2 hours. The mixture was filtered and purified by prep-HPLC [column: Phenomenex luna C18 150×40 mm×15 μm; mobile phase: water(FA)-ACN; gradient: 20%-50% B over 15 minutes] to afford the title compound (130 mg, 50% yield) as white solid; LCMS (ESI, M+1): m/z=648.3.

Step C. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-vinylpiperidin-4-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-vinylpiperidine-4-carboxylic acid (125 mg, 1.0 equiv) and 3-methyl-1H-pyrazole (23.8 mg, 1.5 equiv) in DMF (2 mL) were added HATU (110 mg, 1.5 equiv) and DIEA (125 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 52%-82% B over 20 minutes to afford the title compound (7.76 mg, 5% yield) as off-white solid; ¹H NMR (400 MHz, CHLOROFORM-d) δ=8.93 (s, 1H), 8.15 (d, J=2.8 Hz, 1H), 7.57-7.45 (m, 1H), 7.19-7.07 (m, 2H), 7.00-6.85 (m, 1H), 6.52-6.37 (m, 1H), 6.21 (d, J=2.4 Hz, 1H), 5.40-5.15 (m, 3H), 4.34-4.20 (m, 2H), 4.07-3.89 (m, 4H), 3.37-3.23 (m, 2H), 3.21-3.10 (m, 1H), 3.04-2.95 (m, 1H), 2.72-2.60 (m, 2H), 2.44 (br dd, J=7.6, 14.4 Hz, 1H), 2.36 (br d, J=3.6 Hz, 1H), 2.32 (s, 3H), 2.29-2.08 (m, 5H), 2.00-1.94 (m, 2H), 1.90 (br d, J=8.8 Hz, 1H), 0.80 (t, J=7.2 Hz, 3H); LCMS (ESI, M+1): m/z=712.4.

### Example 103

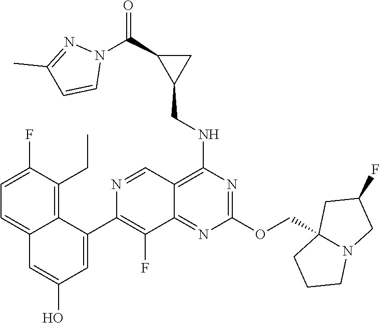 [SMILES: CCc1c(F)ccc2cc(O)cc(-c3ncc4c(NC[C@@H]5C[C@@H]5C(=O)n5ccc(C)n5)nc(OC[C@@]56CCCN5C[C@H](F)C6)nc4c3F)c12]

### ((1S,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropyl)(3-methyl-1H-pyrazol-1-yl)methanone

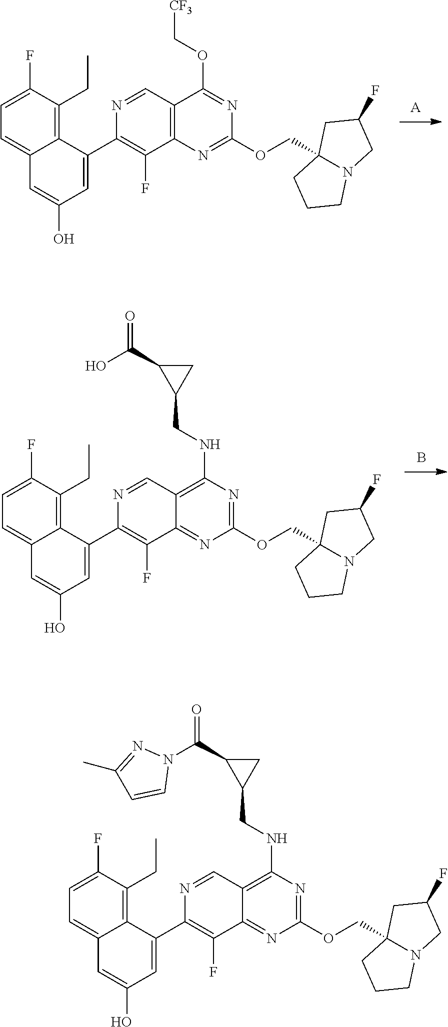

Step A. (1S,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), rac-((1R,2S)-2-(aminomethyl)cyclopropyl)(3-methyl-1H-pyrazol-1-yl)methanone (92.0 mg, 1.5 equiv, HCl) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 5 hours. The mixture was filtered and purified by prep-HPLC [column: Phenomenex luna C18 150×25 mm×10 μm; mobile phase: water(FA)-ACN; gradient: 15%-45% B over 15 minutes] to afford the title compound (77.0 mg, 31% yield) as white solid; LCMS (ESI, M+1): m/z=608.2.

Step B. ((1S,2R)-2-(((7-(8-ethyl-7-fluoro-3-hydroxynaphthalen-1-yl)-8-fluoro-2-yl)amino)methyl)cyclopropyl)(3-methyl-1H-pyrazol-1-yl)methanone: To a solution of (1S,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylic acid (67.0 mg, 1.0 equiv) and 3-methyl-1H-pyrazole (13.6 mg, 1.5 equiv) in DMF (1 mL) were added HATU (63.0 mg, 1.5 equiv) and DIEA (71.0 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 36%-66% B over 20 minutes] to afford the title compound (14.2 mg, 18% yield) as yellow solid; ¹H NMR (400 MHz, CHLOROFORM-d) δ=8.70-8.55 (m, 1H), 8.13-8.03 (m, 1H), 7.53-7.36 (m, 1H), 7.16-7.02 (m, 2H), 6.98-6.86 (m, 1H), 6.26-6.17 (m, 1H), 5.41-5.12 (m, 1H), 4.39-4.03 (m, 3H), 3.67-3.49 (m, 1H), 3.34-3.09 (m, 4H), 3.03-2.93 (m, 1H), 2.49-2.38 (m, 1H), 2.35-2.30 (m, 3H), 2.29-2.18 (m, 2H), 2.11 (br dd, J=7.2, 14.8 Hz, 2H), 1.95 (br d, J=7.6 Hz, 3H), 1.50-1.37 (m, 2H), 1.35-1.26 (m, 1H), 0.78-0.69 (m, 3H); LCMS (ESI, M+1): m/z=672.4.

### Example 104

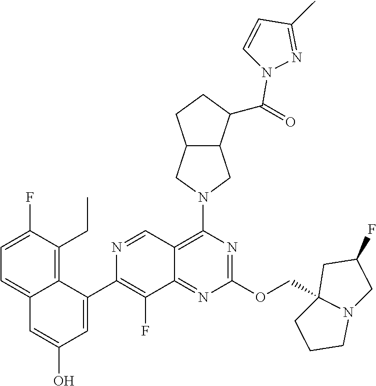 [SMILES: CCc1c(F)ccc2cc(O)cc(-c3ncc4c(N5CC6CCC(C(=O)n7ccc(C)n7)C6C5)nc(OC[C@@]56CCCN5C[C@H](F)C6)nc4c3F)c12]

### (2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-4-yl)(3-methyl-1H-pyrazol-1-yl)methanone

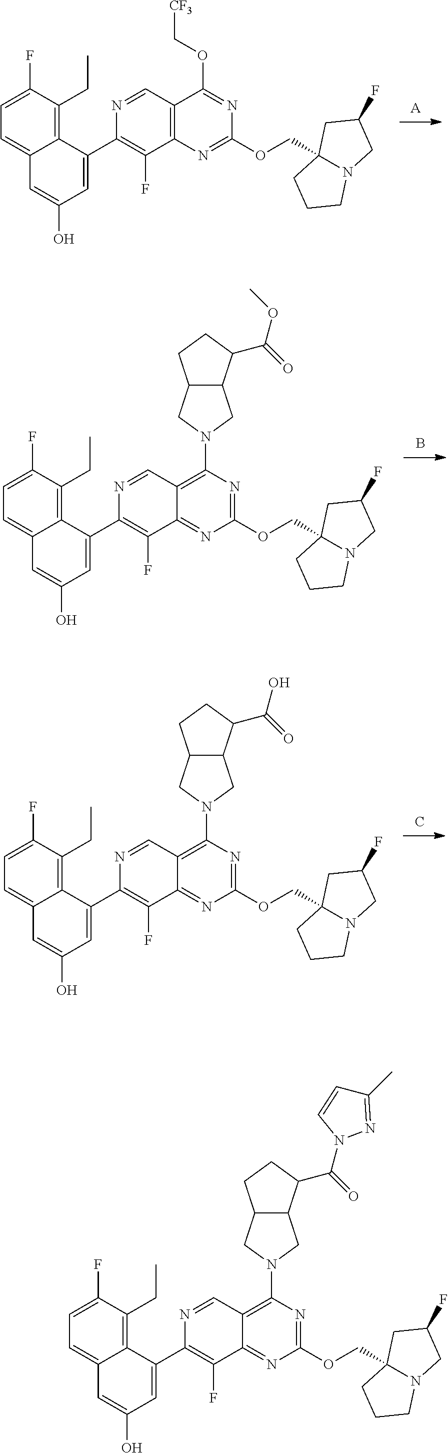

Step A. methyl 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-4-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), methyl octahydrocyclopenta[c]pyrrole-4-carboxylate (125 mg, 1.5 equiv, HCl) and 4 Å molecular sieve (50 mg) in DMF (2 mL) was added K₃PO₄ (258 mg, 3.0 equiv). The reaction was degassed and purged with nitrogen for 3 times. The reaction was stirred at 60 °C for 1 hour. The mixture was quenched with H₂O (10 mL) and extracted with ethyl acetate (10 mL×3). The combined organic layers were washed with brine (20 mL×2), dried over anhydrous sodium sulfate and concentrated under reduced pressure to afford the title compound (280 mg, crude) as yellow oil; LCMS (ESI, M+1): m/z=662.5.

Step B. 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-4-carboxylic acid: To a solution of methyl 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-4-carboxylate (260 mg, 1.0 equiv) in DMSO (3 mL) was added LiOH·H₂O (2 M, 1.0 ml, 5.0 equiv). The reaction was degassed and purged with nitrogen for 3 times. The reaction was stirred at 25 °C for 1 hour. The mixture was filtered and purified by prep-HPLC [column: Phenomenex luna C18 150×40 mm×15 μm; mobile phase: water(FA)-ACN; gradient: 18%-48% B over 15 minutes] to afford the title compound (130 mg, 50% yield) as white solid; LCMS (ESI, M+1): m/z=648.2.

Step C. (2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-4-yl)(3-methyl-1H-pyrazol-1-yl)methanone: To a solution of 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-4-carboxylic acid (125 mg, 1.0 equiv) and 3-methyl-1H-pyrazole (23.8 mg, 1.5 equiv) in DMF (2 mL) were added HATU (110 mg, 1.5 equiv) and DIEA (125 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 48%-78% B over 20 minutes] to afford the title compound (28.9 mg, 21% yield) as yellow solid; ¹H NMR (400 MHz, CHLOROFORM-d) δ=9.18-9.05 (m, 1H), 8.19-8.02 (m, 1H), 7.50 (dd, J=6.0, 9.2 Hz, 1H), 7.20-7.07 (m, 2H), 7.04-6.88 (m, 1H), 6.32-6.20 (m, 1H), 5.39-5.15 (m, 1H), 4.31-4.08 (m, 4H), 4.06-3.74 (m, 3H), 3.44-3.11 (m, 4H), 3.02-2.94 (m, 2H), 2.55-2.36 (m, 2H), 2.32 (d, J=2.8 Hz, 3H), 2.28-2.18 (m, 2H), 2.17-2.06 (m, 4H), 1.99-1.91 (m, 2H), 1.85 (br s, 1H), 1.76-1.68 (m, 1H), 0.85-0.74 (m, 3H); LCMS (ESI, M+1): m/z=712.3.

### Example 105

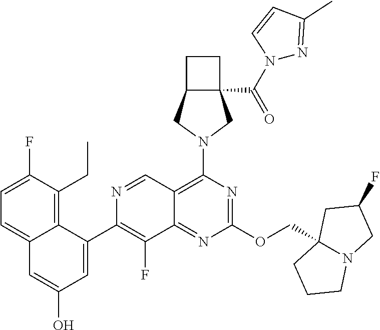 [SMILES: CCc1c(F)ccc2cc(O)cc(-c3ncc4c(N5C[C@H]6CC[C@@]6(C(=O)n6ccc(C)n6)C5)nc(OC[C@@]56CCCN5C[C@H](F)C6)nc4c3F)c12]

### ((1S,5S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-1-yl)(3-methyl-1H-pyrazol-1-yl)methanone

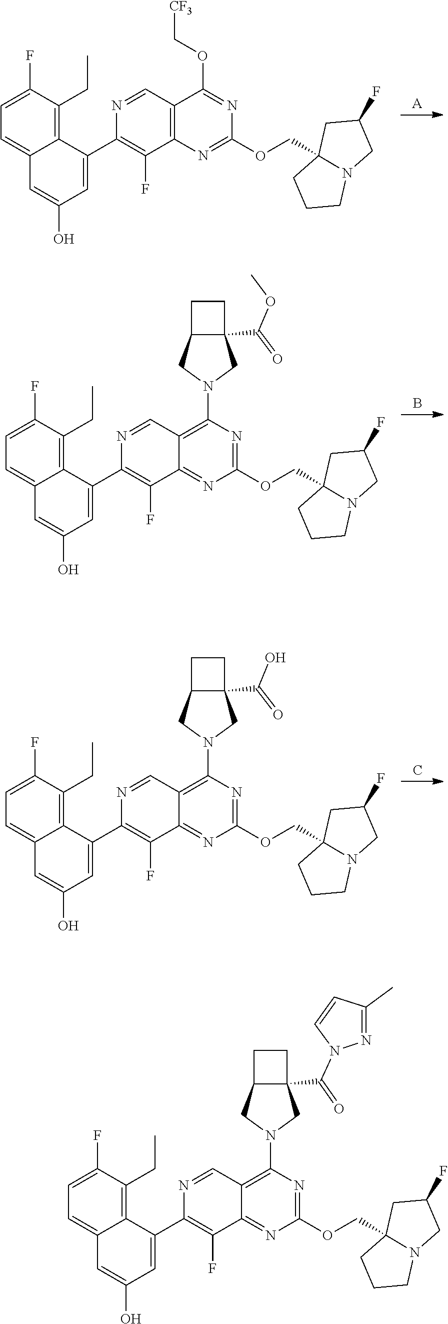

Step A. methyl (1S,5S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-1-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), rac-methyl (1R,5R)-3-azabicyclo[3.2.0]heptane-1-carboxylate (94 mg, 1.5 equiv) and 4 Å molecular sieve (50 mg) in DMF (4 mL) was added K₃PO₄ (258 mg, 3.0 equiv). The reaction was degassed and purged with nitrogen 3 times. The reaction was stirred at 60 °C for 1 hour. The mixture was quenched with H₂O (20 mL) and extracted with ethyl acetate (20 mL×3). The combined organic layers were washed with brine (20 mL×2), dried over anhydrous sodium sulfate and concentrated under reduced pressure to afford the title compound (280 mg, crude) as yellow oil; LCMS (ESI, M+1): m/z=648.2.

Step B. (1S,5S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-1-carboxylic acid: To a solution of methyl (1S,5S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-1-carboxylate (260 mg, 1.0 equiv) in DMSO (3 mL) was added LiOH·H₂O (2 M, 1.0 ml, 5.0 equiv). The reaction was degassed and purged with nitrogen for 3 times. The reaction was stirred at 25 °C for 1 hour. The mixture was filtered and purified by prep-HPLC [column: Phenomenex luna C18 150×40 mm×15 μm; mobile phase: water(FA)-ACN; gradient: 18%-48% B over 15 minutes] to afford the title compound (105 mg, 41% yield) as white solid; LCMS (ESI, M+1): m/z=634.2.

Step C. ((1S,5S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-1-yl)(3-methyl-1H-pyrazol-1-yl)methanone: To a solution of (1S,5S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-1-carboxylic acid (100 mg, 1.0 equiv) and 3-methyl-1H-pyrazole (19.4 mg, 1.5 equiv) in DMF (2 mL) were added HATU (90.0 mg, 1.5 equiv) and DIEA (102 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 48%-78% B over 20 minutes] to afford the title compound (13.6 mg, 12% yield) as white solid; ¹H NMR (400 MHz, CHLOROFORM-d) δ=9.23-9.14 (m, 1H), 8.17-8.09 (m, 1H), 7.59-7.43 (m, 1H), 7.20-7.11 (m, 2H), 7.09-6.96 (m, 1H), 6.25 (d, J=2.8 Hz, 1H), 5.40-5.15 (m, 1H), 4.89-4.70 (m, 1H), 4.45-4.20 (m, 5H), 3.69 (br d, J=6.4 Hz, 1H), 3.37-3.10 (m, 3H), 3.02-2.92 (m, 1H), 2.80-2.66 (m, 1H), 2.51-2.42 (m, 1H), 2.39-2.30 (m, 2H), 2.26 (br s, 1H), 2.22 (d, J=1.6 Hz, 3H), 2.18-2.13 (m, 1H), 2.11-2.06 (m, 1H), 1.99-1.87 (m, 4H), 1.81 (br s, 1H), 0.87-0.77 (m, 3H); LCMS (ESI, M+1): m/z=698.3.

### Example 106

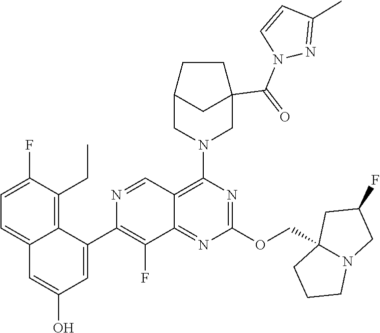 [SMILES: CCc1c(F)ccc2cc(O)cc(-c3ncc4c(N5CC6CCC(C(=O)n7ccc(C)n7)(C6)C5)nc(OC[C@@]56CCCN5C[C@H](F)C6)nc4c3F)c12]

### (3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)(3-methyl-1H-pyrazol-1-yl)methanone

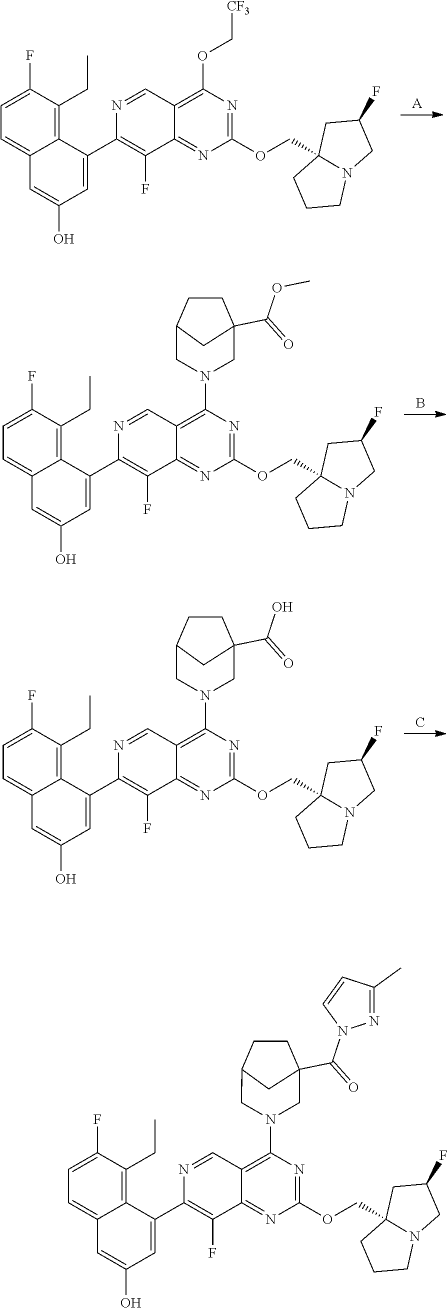

Step A. methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylate: To a solution of methyl 3-azabicyclo[3.2.1]octane-1-carboxylate (208 mg, 2.0 equiv, HCl) in DMF (4 mL) were added DIEA (327 mg, 5.0 equiv) and 4 Å molecular sieve (150 mg) slowly at 25 °C. The reaction was stirred at 25 °C for 30 minutes. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and K₃PO₄ (322 mg, 3.0 equiv) were added at 25 °C slowly. The reaction was stirred at 60 °C for 2 hours. The mixture was filtered and purified by reversed phase flash [C18, 0.1% formic acid condition] to afford the title compound (270 mg, 72% yield, HCOOH salt) as white solid; LCMS (ESI, M+1): m/z=662.3.

Step B. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylic acid: To a solution of methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylate (200 mg, 1.0 equiv, HCOOH) in MeOH (2 mL) were added LiOH·H₂O (2 M, 2 ml, 14 equiv). The reaction was stirred at 25 °C for 30 minutes. The mixture was filtered and purified by reversed phase flash [C18, 0.1% formic acid condition] to afford the title compound (110 mg, 55% yield, HCOOH salt) as white solid; LCMS (ESI, M+1): m/z=648.4.

Step C. (3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)(3-methyl-1H-pyrazol-1-yl)methanone: To a solution of 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylic acid (50.0 mg, 1.0 equiv, HCOOH) and 3-methyl-1H-pyrazole (17.7 mg, 3.0 equiv) in DMF (0.5 mL) were added HATU (82.2 mg, 3.0 equiv) and DIEA (55.9 mg, 6.0 equiv). The reaction was stirred at 25 °C for 1 hour. The mixture was filtered and purified by reversed phase flash [Waters Xbridge 150×25 mm×5 μm; A: water (NH₄HCO₃), B: ACN, B %: 60%-90% over 9 minutes] to afford the title compound (18.5 mg, 35% yield) as yellow solid; ¹H NMR (400 MHz, CHLOROFORM-d) δ=9.26-9.16 (m, 1H), 8.21-8.08 (m, 1H), 7.57-7.43 (m, 1H), 6.99 (s, 3H), 6.26-6.13 (m, 1H), 5.55-5.13 (m, 2H), 4.68-4.40 (m, 1H), 4.33-4.22 (m, 2H), 3.77-3.62 (m, 1H), 3.46-3.05 (m, 5H), 3.05-2.93 (m, 1H), 2.59-2.46 (m, 2H), 2.45-2.37 (m, 2H), 2.36-2.32 (m, 1H), 2.26 (br d, J=3.2 Hz, 2H), 2.21 (s, 6H), 2.00-1.90 (m, 3H), 1.76-1.62 (m, 1H), 0.86-0.73 (m, 3H); LCMS (ESI, M+1): m/z=712.3.

### Example 107

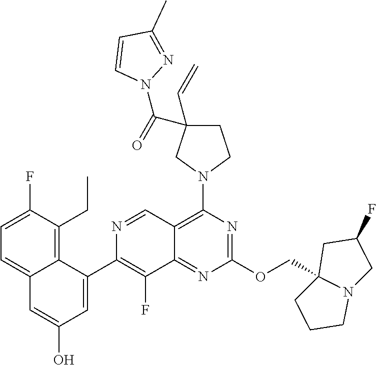 [SMILES: C=CC1(C(=O)n2ccc(C)n2)CCN(c2nc(OC[C@@]34CCCN3C[C@H](F)C4)nc3c(F)c(-c4cc(O)cc5ccc(F)c(CC)c45)ncc23)C1]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-vinylpyrrolidin-3-yl)(3-methyl-1H-pyrazol-1-yl)methanone

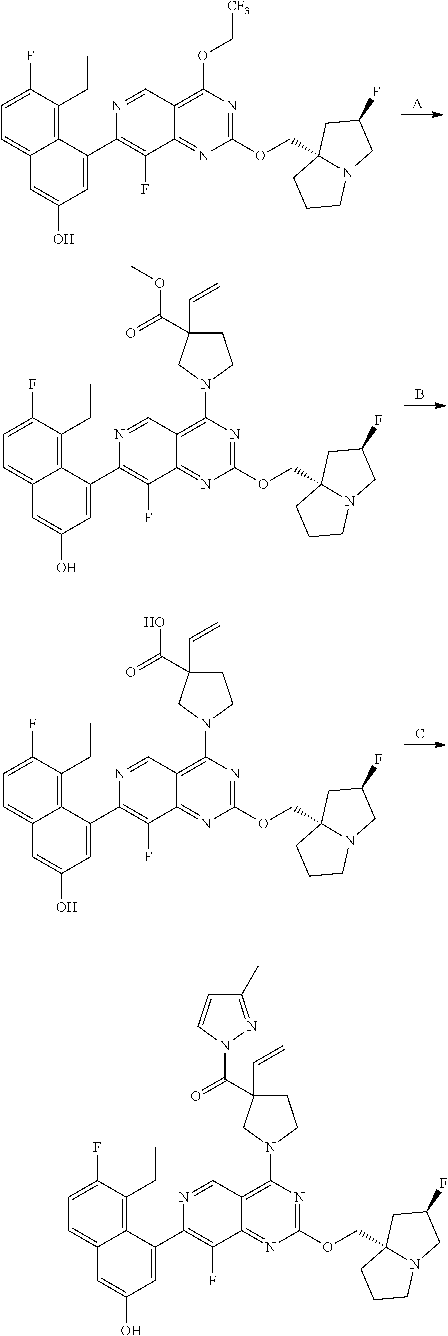

Step A. methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-vinylpyrrolidine-3-carboxylate: To a solution of methyl 3-vinylpyrrolidine-3-carboxylate (194 mg, 2.0 equiv, HCl) in DMF (3 mL) was added DIEA (327 mg, 5.0 equiv) slowly at 30 °C. The reaction was stirred at 30 °C for 30 minutes. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) was added at 30 °C slowly. The reaction was stirred at 30 °C for 3 hours. The mixture was filtered and purified by reversed phase flash [C18, 0.1% formic acid condition] to afford the title compound (280 mg, 76% yield, HCOOH salt) as white solid; LCMS (ESI, M+1): m/z=648.4.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-vinylpyrrolidine-3-carboxylic acid: To a solution of methyl 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-vinylpyrrolidine-3-carboxylate (200 mg, 1.0 equiv, HCOOH) in MeOH (2 mL) were added LiOH·H₂O (2 M, 2 mL, 14 equiv). The reaction was stirred at 25 °C for 30 minutes. The mixture was filtered and purified by reversed phase flash [C18, 0.1% formic acid condition] to afford the title compound (110 mg, 52% yield, HCOOH salt) as white solid; LCMS (ESI, M+1): m/z=634.2.

Step C. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-vinylpyrrolidin-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-vinylpyrrolidine-3-carboxylic acid (50.0 mg, 1.0 equiv) and 3-methyl-1H-pyrazole (18.1 mg, 3.0 equiv) in DMF (0.5 mL) were added HATU (83.9 mg, 3.0 equiv) and DIEA (57.0 mg, 6.0 equiv). The reaction was stirred at 25 °C for 1 hour. The mixture was filtered and purified by reversed phase flash [Waters Xbridge 150×25 mm×5 μm; A: water (NH₄HCO₃), B: ACN, B %: 55%-85% over 9 minutes] to afford the title compound (37.0 mg, 71% yield) as yellow solid; ¹H NMR (400 MHz, CHLOROFORM-d) δ=9.10 (br d, J=16.4 Hz, 1H), 8.13 (dd, J=2.4, 6.4 Hz, 1H), 7.49-7.39 (m, 1H), 7.20-7.02 (m, 2H), 7.00-6.73 (m, 1H), 6.44-6.28 (m, 1H), 6.27-6.21 (m, 1H), 5.40-5.16 (m, 3H), 4.60-4.48 (m, 1H), 4.37-4.22 (m, 2H), 4.08-3.88 (m, 2H), 3.43-3.05 (m, 4H), 3.03-2.94 (m, 1H), 2.87-2.70 (m, 1H), 2.62-2.38 (m, 3H), 2.36-2.24 (m, 4H), 2.22-2.06 (m, 3H), 2.00 (s, 2H), 0.86-0.70 (m, 3H); LCMS (ESI, M+1): m/z=698.2.

### Example 108

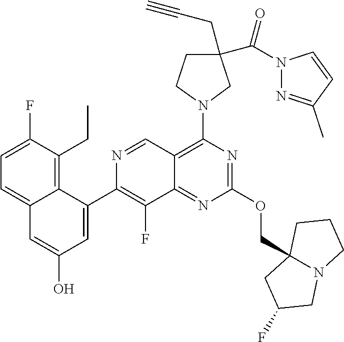 [SMILES: C#CCC1(C(=O)n2ccc(C)n2)CCN(c2nc(OC[C@@]34CCCN3C[C@H](F)C4)nc3c(F)c(-c4cc(O)cc5ccc(F)c(CC)c45)ncc23)C1]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(prop-2-yn-1-yl) pyrrolidin-3-yl)(3-methyl-1H-pyrazol-1-yl)methanone

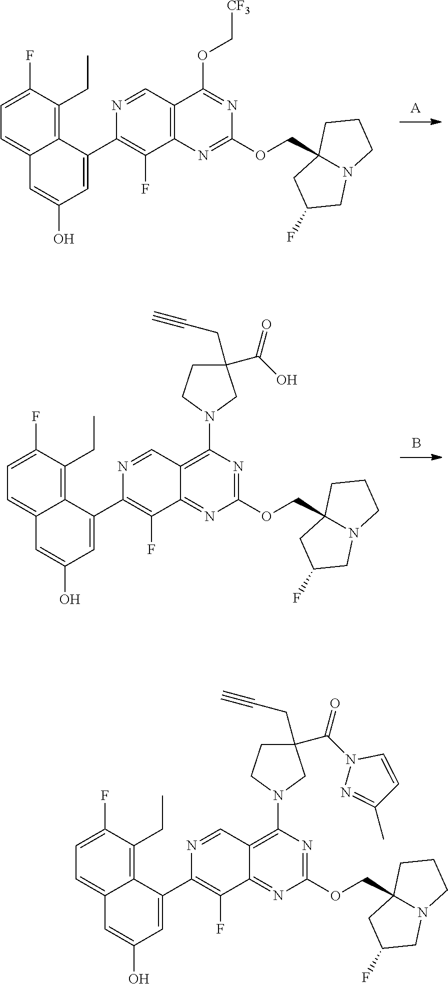

Step A. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(prop-2-yn-1-yl) pyrrolidine-3-carboxylic acid: To a solution of 3-(prop-2-yn-1-yl) pyrrolidine-3-carboxylic acid (192 mg, 2.0 equiv, HCl) in DMF (4 mL) were added DIEA (327 mg, 5.0 equiv) and 4 Å molecular sieve (150 mg) slowly at 25 °C. The reaction was stirred at 25 °C for 0 minutes. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and K₃PO₄ (322 mg, 3.0 equiv) were added at 25 °C slowly. The reaction was stirred at 60 °C for 1 hour. The mixture was filtered and purified by reversed phase flash [C18, 0.1% formic acid condition] to afford the title compound (120 mg, 34% yield, HCOOH salt) as white solid; LCMS (ESI, M+1): m/z=646.3.

Step B. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(prop-2-yn-1-yl) pyrrolidin-3-yl)(3-methyl-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-(prop-2-yn-1-yl) pyrrolidine-3-carboxylic acid (50.0 mg, 1.0 equiv, HCOOH) and 3-methyl-1H-pyrazole (17.8 mg, 3.0 equiv) in DMF (0.5 mL) were added HATU (82.5 mg, 3.0 equiv) and DIEA (56.0 mg, 6.0 equiv). The reaction was stirred at 25 °C for 1 hour. The mixture was filtered and purified by reversed phase flash [Waters Xbridge 150×25 mm×5 μm; A: water (NH₄HCO₃), B: ACN, B %: 54%-84% over 9 minutes] to afford the title compound (11.7 mg, 22% yield) as white solid; ¹H NMR (400 MHz, CHLOROFORM-d) δ=9.13 (br d, J=8.0 Hz, 1H), 8.16 (br s, 1H), 7.62-7.44 (m, 1H), 7.21-7.05 (m, 2H), 7.01-6.85 (m, 1H), 6.26 (s, 1H), 5.43-5.15 (m, 1H), 4.73-4.54 (m, 1H), 4.46-4.19 (m, 3H), 4.07-3.96 (m, 1H), 3.43-3.26 (m, 2H), 3.26-3.09 (m, 3H), 3.04-2.96 (m, 1H), 2.89-2.57 (m, 2H), 2.55-2.37 (m, 3H), 2.27 (br s, 4H), 2.25-2.07 (m, 4H), 2.03-1.96 (m, 2H), 0.85-0.75 (m, 3H); LCMS (ESI, M+1): m/z=710.3.

### Example 109

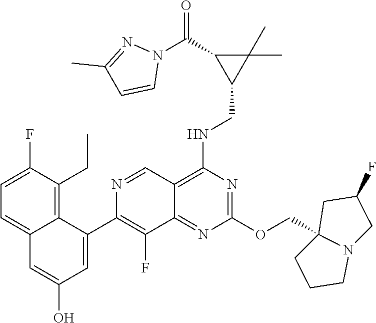 [SMILES: CCc1c(F)ccc2cc(O)cc(-c3ncc4c(NC[C@@H]5[C@H](C(=O)n6ccc(C)n6)C5(C)C)nc(OC[C@@]56CCCN5C[C@H](F)C6)nc4c3F)c12]

### ((1S,3R)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-2,2-dimethylcyclopropyl)(3-methyl-1H-pyrazol-1-yl)methanone

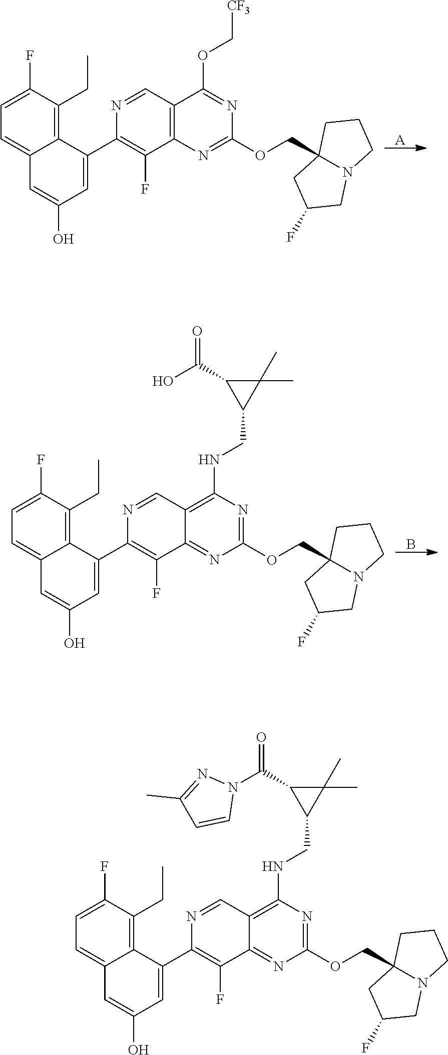

Step A. (1S,3R)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-2,2-dimethylcyclopropane-1-carboxylic acid: To a solution of (1S,3R)-3-(aminomethyl)-2,2-dimethylcyclopropane-1-carboxylic acid (182 mg, 2.0 equiv, HCl) in DMF (4 mL) were added DIEA (327 mg, 5.0 equiv) and 4 Å molecular sieve (150 mg) slowly at 25 °C. The reaction was stirred at 25 °C for 30 minutes. 5-ethyl-6- fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (300 mg, 1.0 equiv) and K₃PO₄ (322 mg, 3.0 equiv) were added at 25 °C slowly. The reaction was stirred at 60 °C for 6 hours. The mixture was filtered and purified by reversed phase flash [Waters Xbridge 150×25 mm×10 μm; A: water(HCOOH), B: ACN, B %: 18%-48% over 1 minute] to afford the title compound (110 mg, 31% yield, HCOOH salt) as white solid; LCMS (ESI, M+1): m/z=636.4.

Step B. ((1S,3R)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-2,2-dimethylcyclopropyl)(3-methyl-1H-pyrazol-1-yl)methanone: To a solution of (1S,3R)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-2,2-dimethylcyclopropane-1-carboxylic acid (50.0 mg, 1.0 equiv, HCOOH) and 3-methyl-1H-pyrazole (18.1 mg, 3.0 equiv) in DMF (0.5 mL) were added HATU (83.7 mg, 3.0 equiv) and DIEA (56.9 mg, 6.0 equiv). The reaction was stirred at 25 °C for 30 minutes. The mixture was filtered and purified by reversed phase flash [Waters Xbridge 150×25 mm×5 μm; A: water (NH₄HCO₃), B: ACN, B %: 45%-75% over 15 minutes] to afford the title compound (15.1 mg, 29% yield) as yellow solid; ¹H NMR (400 MHz, CHLOROFORM-d) δ=8.85-8.60 (m, 1H), 8.20-8.04 (m, 1H), 7.44 (br d, J=5.6 Hz, 1H), 7.19-6.96 (m, 3H), 6.24 (br dd, J=3.6, 8.4 Hz, 1H), 5.39-5.17 (m, 1H), 4.51-4.36 (m, 1H), 4.28 (br s, 2H), 3.82-3.53 (m, 1H), 3.39-3.22 (m, 2H), 3.21-3.10 (m, 1H), 3.02-2.85 (m, 2H), 2.59-2.38 (m, 2H), 2.37-2.24 (m, 5H), 2.23-2.08 (m, 3H), 1.95 (br s, 2H), 1.37-1.31 (m, 6H), 0.80-0.69 (m, 3H); LCMS (ESI, M+1): m/z=700.3.

### Example 110

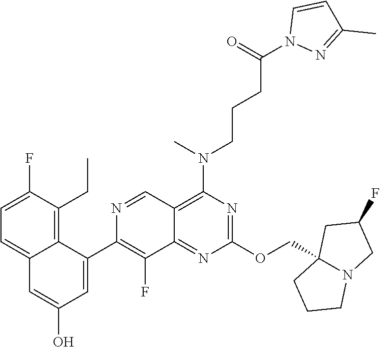 [SMILES: CCc1c(F)ccc2cc(O)cc(-c3ncc4c(N(C)CCCC(=O)n5ccc(C)n5)nc(OC[C@@]56CCCN5C[C@H](F)C6)nc4c3F)c12]

### 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-1-(3-methyl-1H-pyrazol-1-yl) butan-1-one

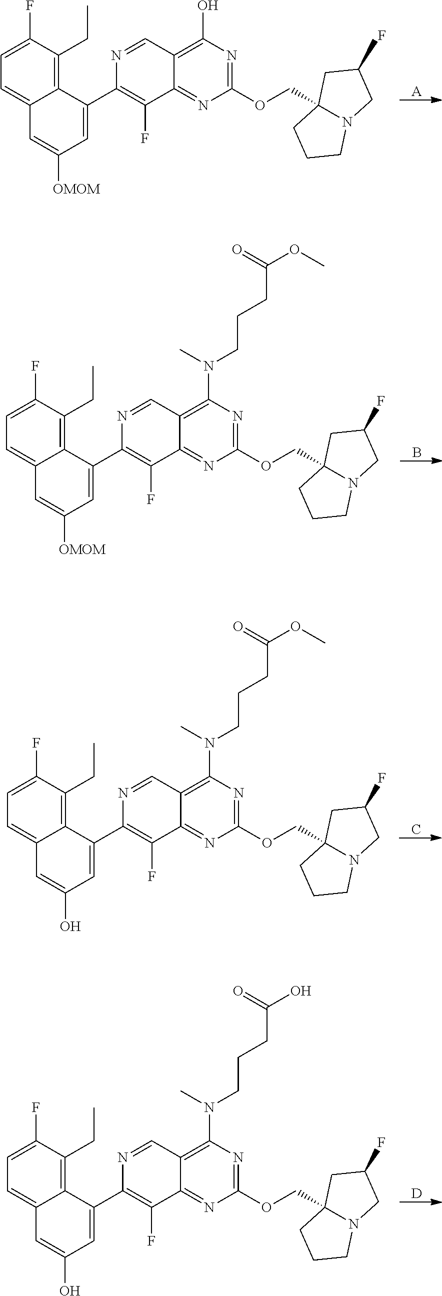

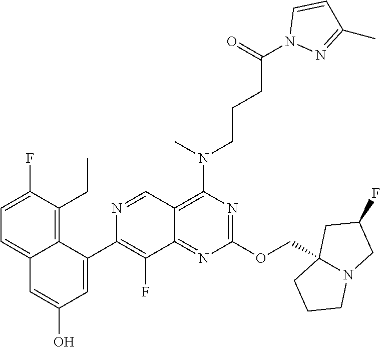 [SMILES: CCc1c(F)ccc2cc(O)cc(-c3ncc4c(N(C)CCCC(=O)n5ccc(C)n5)nc(OC[C@@]56CCCN5C[C@H](F)C6)nc4c3F)c12]

Step A. methyl 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (800 mg, 1.0 equiv) in DMF (10 mL) were added DIEA (932 mg, 5.0 equiv) and PyBOP (1.13 g, 1.5 equiv) at 25 °C. The reaction was stirred at 25 °C for 0.5 hours. Then methyl 4-(methylamino)butanoate (362 mg, 1.5 equiv, HCl) was added. The reaction was stirred at 25 °C for 12 hours. The mixture was diluted with water (50 mL) and extracted with ethyl acetate (30 mL×2). The combined organic layers were washed with brine (60 mL), dried over anhydrous sodium sulfate, concentrated and purified by column chromatography [SiO₂, petroleum ether:ethyl acetate=1:1 to 0:1] to afford the title compound (624 mg, 64% yield) as white solid; LCMS (ESI, M+1): m/z=668.3.

Step B. methyl 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoate: To a solution of methyl 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoate (624 mg, 1.0 equiv) in MeCN (7 mL) was added HCl·dioxane (7 mL, 2 M). The reaction was stirred at 0 °C for 1 hour. The mixture was concentrated under reduced pressure to remove solvent. The mixture was diluted with saturated NaHCO₃ solution (15 mL) and extracted with ethyl acetate (15 mL×2). The combined organic layers were washed with brine (20 mL), dried over anhydrous sodium sulfate and concentrated to afford the title compound (491 mg, 80% yield) as off-white solid; LCMS (ESI, M+1): m/z=624.3.

Step C. 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoic acid: To a solution of methyl 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoate (491 mg, 1.0 equiv) in EtOH (5 mL) was added NaOH (1.2 mL, 3.0 equiv, 2 M). The reaction was stirred at 25 °C for 1 hour. The mixture was quenched with saturated citric acid solution (3 mL), then diluted with water (10 mL) and extracted with ethyl acetate (10 mL×2). The combined organic layers were washed with brine (20 mL), dried over anhydrous sodium sulfate, concentrated and purified by prep-HPLC [column: Phenomenex luna C18 150×40 mm×15 μm; mobile phase: [water(FA)-ACN]; gradient: 10%-40% B over 15 minute] to afford the title compound (360 mg, 75% yield) as off-white solid; LCMS (ESI, M+1): m/z=610.2.

Step D. 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-1-(3-methyl-1H-pyrazol-1-yl) butan-1-one: To a solution of 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoic acid (60.0 mg, 1.0 equiv) in DMF (1 mL) were added HATU (56.1 mg, 1.5 equiv), 3-methyl-1H-pyrazole (12.1 mg, 1.5 equiv) and DIEA (63.6 mg, 5.0 equiv). The reaction was stirred at 25 °C for 1 hour. The mixture was purified by prep-HPLC [column: Waters Xbridge Prep OBD C18 150×40 mm×10 μm; mobile phase: [water (NH₄HCO₃)-ACN]; gradient: 38%-68% B over 20 minute] to afford the title compound (33.0 mg, 48% yield) as off-white solid; ¹H NMR (400 MHz, CHLOROFORM-d) δ=9.07 (d, J=12.4 Hz, 1H), 8.09 (d, J=2.8 Hz, 1H), 7.47 (dt, J=2.8, 6.0 Hz, 1H), 7.17-7.08 (m, 2H), 7.06-6.94 (m, 1H), 6.22 (d, J=2.8 Hz, 1H), 5.40-5.18 (m, 1H), 4.34-4.17 (m, 2H), 3.96-3.80 (m, 2H), 3.45 (d, J=11.6 Hz, 3H), 3.38-3.24 (m, 2H), 3.23-3.18 (m, 2H), 3.17-3.06 (m, 1H), 3.03-2.94 (m, 1H), 2.56-2.36 (m, 2H), 2.28 (s, 3H), 2.26-2.18 (m, 3H), 2.16-2.08 (m, 2H), 1.98-1.87 (m, 3H), 0.85-0.77 (m, 3H); LCMS (ESI, M+1): m/z=674.4.

### Example 111

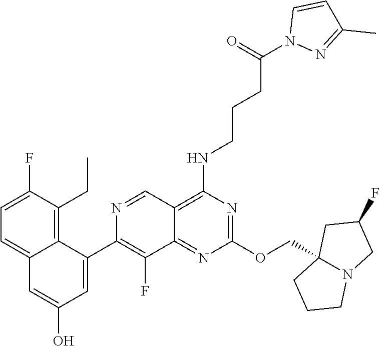 [SMILES: CCc1c(F)ccc2cc(O)cc(-c3ncc4c(NCCCC(=O)n5ccc(C)n5)nc(OC[C@@]56CCCN5C[C@H](F)C6)nc4c3F)c12]

### 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) butan-1-one

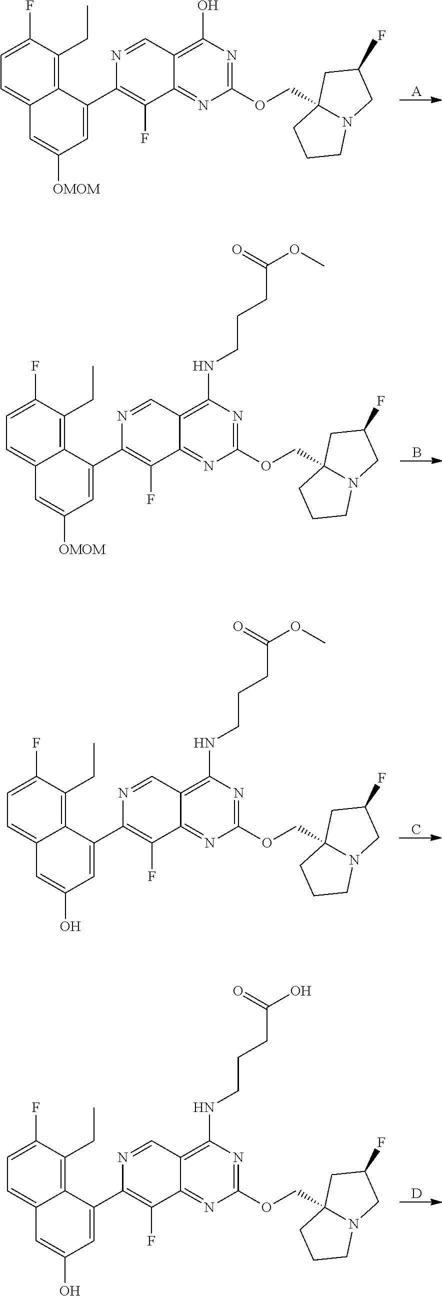

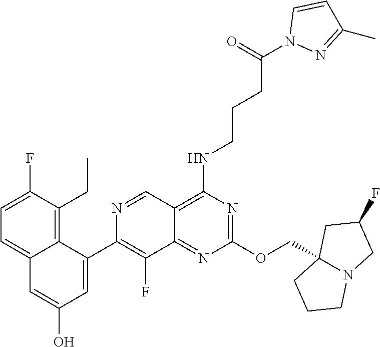 [SMILES: CCc1c(F)ccc2cc(O)cc(-c3ncc4c(NCCCC(=O)n5ccc(C)n5)nc(OC[C@@]56CCCN5C[C@H](F)C6)nc4c3F)c12]

Step A. methyl 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (600 mg, 1.0 equiv) in DMF (8 mL) were added DIEA (699 mg, 5.0 equiv) and PyBOP (844 mg, 1.5 equiv) at 25 °C. The reaction was stirred at 25 °C for 0.5 hours. Then methyl 4-aminobutanoate (249 mg, 1.5 equiv, HCl) was added. The reaction was stirred at 25 °C for 12 hours. The mixture was diluted with water (50 mL) and extracted with ethyl acetate (30 mL×2). The combined organic layers were washed with brine (30 mL), dried over anhydrous sodium sulfate, concentrated and purified by column chromatography [SiO₂, petroleum ether:ethyl acetate=1:1 to 0:1] to afford the title compound (690 mg, 94% yield) as yellow oil; LCMS (ESI, M+1): m/z=654.3.

Step B. methyl 4-((7-(8-ethyl-7-fluoro-3-hydroxynaphthalen-1-yl)-8-fluoro-2-yl)amino)butanoate: To a of solution methyl 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate (624 mg, 1.0 equiv) in MeCN (7 mL) was added HCl·dioxane (7 mL, 2 M). The reaction was stirred at 0 °C for 1 hour. The mixture was concentrated under reduced pressure to remove solvent. The mixture was diluted with saturated NaHCO₃ solution (10 mL) and extracted with ethyl acetate (10 mL×2). The combined organic layers were washed with brine (20 mL), dried over anhydrous sodium sulfate and concentrated to afford the title compound (610 mg, 76% yield) as yellow oil; LCMS (ESI, M+1): m/z=610.2.

Step C. 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoic acid: To a solution of methyl 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate (610 mg, 1.0 equiv) in EtOH (6 mL) was added NaOH (1.5 mL, 3.0 equiv, 2 M). The reaction was stirred at 25 °C for 1 hour. The mixture was quenched with saturated citric acid solution (4 mL), then diluted with water (20 mL) and extracted with ethyl acetate (10 mL×2). The combined organic layers were washed with brine (20 mL), dried over anhydrous sodium sulfate, concentrated and purified by prep-HPLC [column: Phenomenex luna C18 150×40 mm×15 μm; mobile phase: [water(FA)-ACN]; gradient: 13%-43% B over 15 minutes] to afford the title compound (235 mg, 39% yield) as off-white solid; LCMS (ESI, M+1): m/z=596.2.

Step D. 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) butan-1-one: To a solution of 4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoic acid (60.0 mg, 1.0 equiv) in DMF (1 mL) were added HATU (57.4 mg, 1.5 equiv), 3-methyl-1H-pyrazole (12.4 mg, 1.5 equiv) and DIEA (65.1 mg, 5.0 equiv). The reaction was stirred at 25 °C for 1 hour. The mixture was purified by prep-HPLC [column: Waters Xbridge Prep OBD C18 150×40 mm×10 μm; mobile phase: [water (NH₄HCO₃)-ACN]; gradient: 38%-68% B over 20 minute] to afford the title compound (29.2 mg, 41% yield) as off-white solid; ¹H NMR (400 MHz, CHLOROFORM-d) δ=9.01-8.82 (m, 1H), 8.12 (dd, J=2.8, 5.6 Hz, 1H), 7.82-7.66 (m, 1H), 7.49-7.38 (m, 1H), 7.10 (dt, J=3.2, 9.2 Hz, 1H), 7.06-6.97 (m, 1H), 6.96-6.80 (m, 1H), 6.24 (d, J=2.4 Hz, 1H), 5.37-5.18 (m, 1H), 4.31-4.21 (m, 2H), 3.71-3.48 (m, 2H), 3.37-3.22 (m, 2H), 3.22-3.13 (m, 3H), 3.02-2.95 (m, 1H), 2.53-2.35 (m, 2H), 2.30 (s, 3H), 2.27-2.18 (m, 2H), 2.10 (br d, J=5.6 Hz, 2H), 1.99-1.86 (m, 4H), 0.74 (br t, J=7.6 Hz, 3H); LCMS (ESI, M+1): m/z=660.4.

### Example 112

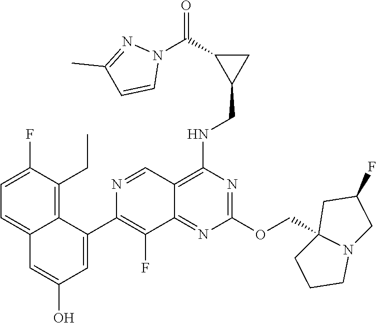 [SMILES: CCc1c(F)ccc2cc(O)cc(-c3ncc4c(NC[C@@H]5C[C@H]5C(=O)n5ccc(C)n5)nc(OC[C@@]56CCCN5C[C@H](F)C6)nc4c3F)c12]

### ((1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropyl)(3-methyl-1H-pyrazol-1-yl)methanone

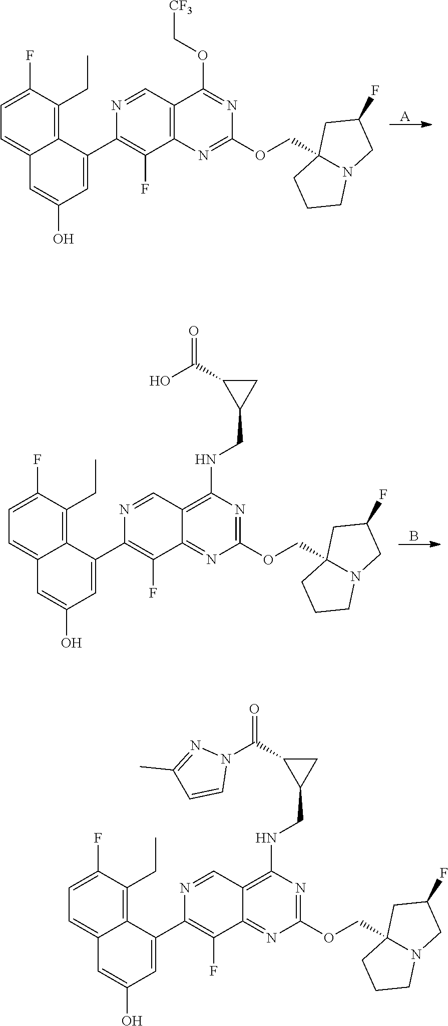

Step A. (1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), (1R,2R)-2-(aminomethyl)cyclopropane-1-carboxylic acid (70 mg, 1.5 equiv) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 1 hour. The mixture was filtered and purified by [column: Phenomenex luna C18 150×40 mm×15 μm; mobile phase: water(FA)-ACN; gradient: 13%-43% B over 15 minutes] to afford the title compound (200 mg, 81% yield) as white solid; LCMS (ESI, M+1): m/z=608.3.

Step B. ((1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropyl)(3-methyl-1H-pyrazol-1-yl)methanone: To a solution of (1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylic acid (195 mg, 1.0 equiv) and 3-methyl-1H-pyrazole (39.5 mg, 1.5 equiv) in DMF (2 mL) were added HATU (183 mg, 1.5 equiv) and DIEA (207 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 38%-68% B over 20 minutes] to afford the title compound (47.3 mg, 21% yield) as yellow solid; ¹H NMR (400 MHz, DMSO-d₆) δ=9.91 (br s, 1H), 9.29 (s, 1H), 9.18 (br s, 1H), 8.27 (br s, 1H), 7.76 (dd, J=6.0, 8.8 Hz, 1H), 7.40-7.28 (m, 2H), 6.99 (d, J=2.0 Hz, 1H), 6.47 (d, J=1.6 Hz, 1H), 5.37-5.15 (m, 1H), 4.22-4.00 (m, 2H), 3.84-3.52 (m, 2H), 3.19-3.05 (m, 3H), 3.00 (s, 1H), 2.88-2.75 (m, 1H), 2.39-2.30 (m, 1H), 2.25 (s, 3H), 2.15-1.97 (m, 5H), 1.88-1.72 (m, 3H), 1.43-1.30 (m, 2H), 0.75-0.65 (m, 3H); LCMS (ESI, M+1): m/z=672.3.

### Example 113

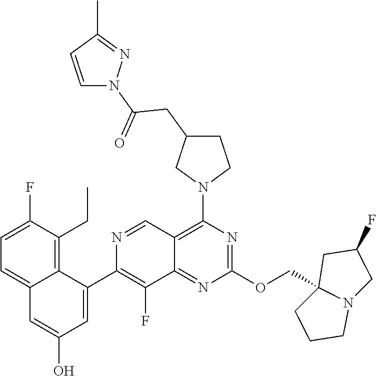 [SMILES: CCc1c(F)ccc2cc(O)cc(-c3ncc4c(N5CCC(CC(=O)n6ccc(C)n6)C5)nc(OC[C@@]56CCCN5C[C@H](F)C6)nc4c3F)c12]

### 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidin-3-yl)-1-(3-methyl-1H-pyrazol-1-yl) ethan-1-one

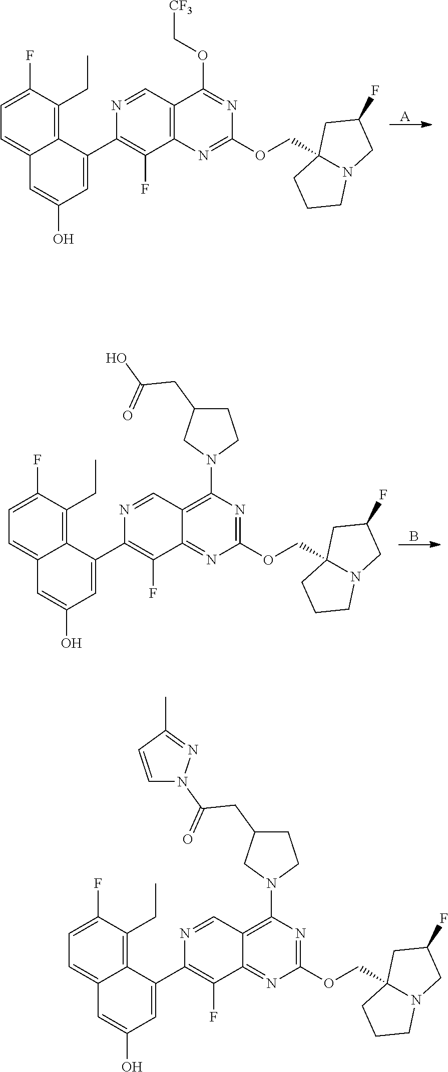

Step A. 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidin-3-yl) acetic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), 2-(pyrrolidin-3-yl) acetic acid (78.0 mg, 1.5 equiv) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 1 hour. The mixture was filtered and purified by perp-HPLC [column: Phenomenex luna C18 150×40 mm×15 μm; mobile phase: water(FA)-ACN; gradient: 13%-43% B over 15 minutes] to afford the title compound (160 mg, 63% yield) as white solid; LCMS (ESI, M+1): m/z=622.4.

Step B. 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidin-3-yl)-1-(3-methyl-1H-pyrazol-1-yl) ethan-1-one: To a solution of 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) pyrrolidin-3-yl) acetic acid (155 mg, 1.0 equiv) and 3-methyl-1H-pyrazole (30.7 mg, 1.5 equiv) in DMF (2 mL) were added HATU (142 mg, 1.5 equiv) and DIEA (161 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 42%-72% B over 20 minutes] to afford the title compound (72.3 mg, 39% yield) as off-white solid; ¹H NMR (400 MHz, CHLOROFORM-d) δ=9.14-9.00 (m, 1H), 8.22-8.09 (m, 1H), 7.51-7.40 (m, 1H), 7.20-7.02 (m, 2H), 6.90 (br d, J=7.6 Hz, 1H), 6.28 (d, J=1.6 Hz, 1H), 5.46-5.11 (m, 1H), 4.36-4.14 (m, 3H), 4.11-3.99 (m, 1H), 3.95-3.51 (m, 2H), 3.39-3.15 (m, 5H), 3.05-2.88 (m, 2H), 2.45 (br dd, J=6.4, 12.8 Hz, 2H), 2.32 (br d, J=3.2 Hz, 3H), 2.25 (br d, J=16.4 Hz, 2H), 2.13 (br t, J=11.6 Hz, 3H), 2.02-1.90 (m, 3H), 0.79 (q, J=6.8 Hz, 3H); LCMS (ESI, M+1): m/z=686.3.

### Example 114

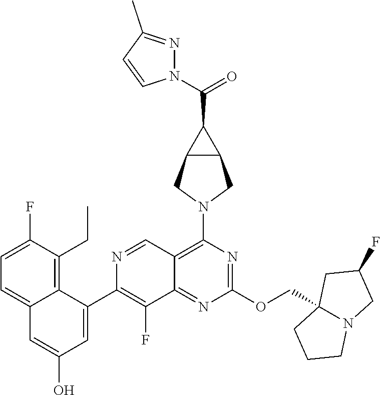 [SMILES: CCc1c(F)ccc2cc(O)cc(-c3ncc4c(N5C[C@@H]6[C@H](C5)[C@@H]6C(=O)n5ccc(C)n5)nc(OC[C@@]56CCCN5C[C@H](F)C6)nc4c3F)c12]

### ((1R,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl)(3-methyl-1H-pyrazol-1-yl)methanone

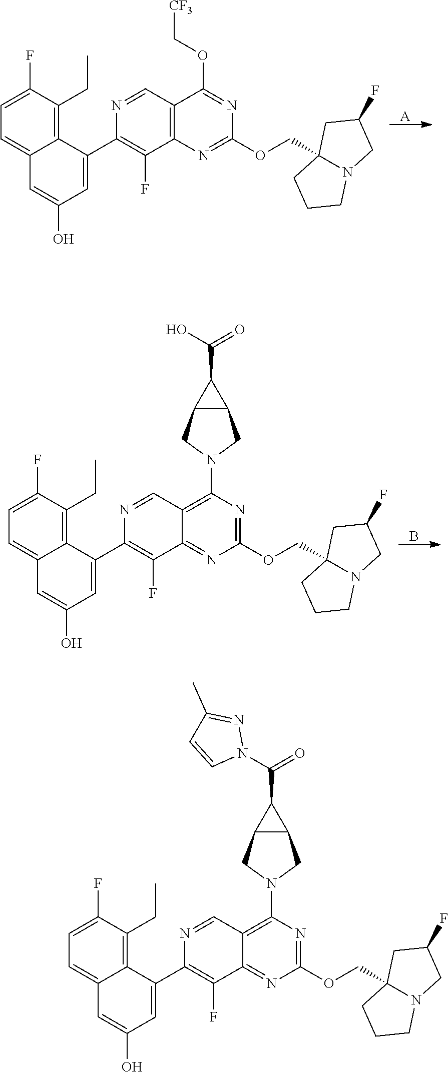

Step A. (1R,5S,6R)-3-(7-(8-ethyl-7-fluoro-3-hydroxynaphthalen-1-yl)-8-fluoro-2-yl)-3-azabicyclo[3.1.0]hexane-6-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), (1R,5S,6s)-3-azabicyclo[3.1.0]hexane-6-carboxylic acid (99.4 mg, 1.5 equiv, HCl) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 1 hour. The mixture was filtered and purified by prep-HPLC [column: Phenomenex luna C18 150×40 mm×15 μm; mobile phase: water(FA)-ACN; gradient: 13%-43% B over 15 minutes] to afford the title compound (125 mg, 50% yield) as white solid; LCMS (ESI, M+1): m/z=620.2.

Step B. ((1R,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl)(3-methyl-1H-pyrazol-1-yl)methanone: To a solution of (1R,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexane-6-carboxylic acid (120 mg, 1.0 equiv) and 3-methyl-1H-pyrazole (23.8 mg, 1.5 equiv) in DMF (2 mL) were added HATU (110 mg, 1.5 equiv) and DIEA (125 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 38%-68% B over 20 minutes] to afford the title compound (56.7 mg, 42% yield) as off-white solid; ¹H NMR (400 MHz, CHLOROFORM-d) δ=9.20-9.04 (m, 1H), 8.02 (br s, 1H), 7.52-7.39 (m, 1H), 7.17-7.06 (m, 2H), 7.03-6.95 (m, 1H), 6.25 (s, 1H), 5.38-5.12 (m, 1H), 4.40-4.10 (m, 6H), 3.35-3.19 (m, 3H), 3.18-3.08 (m, 1H), 3.01-2.92 (m, 1H), 2.59-2.42 (m, 3H), 2.34 (s, 3H), 2.30-2.23 (m, 1H), 2.22-2.14 (m, 2H), 2.10 (br d, J=7.2 Hz, 1H), 1.96-1.89 (m, 2H), 1.87-1.81 (m, 1H), 0.81 (br t, J=6.8 Hz, 3H); LCMS (ESI, M+1): m/z=684.3.

### Example 115

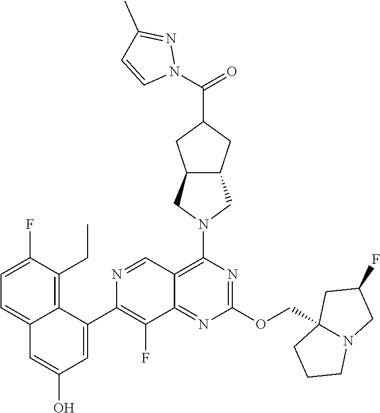 [SMILES: CCc1c(F)ccc2cc(O)cc(-c3ncc4c(N5C[C@H]6CC(C(=O)n7ccc(C)n7)C[C@@H]6C5)nc(OC[C@@]56CCCN5C[C@H](F)C6)nc4c3F)c12]

### ((3aS,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone

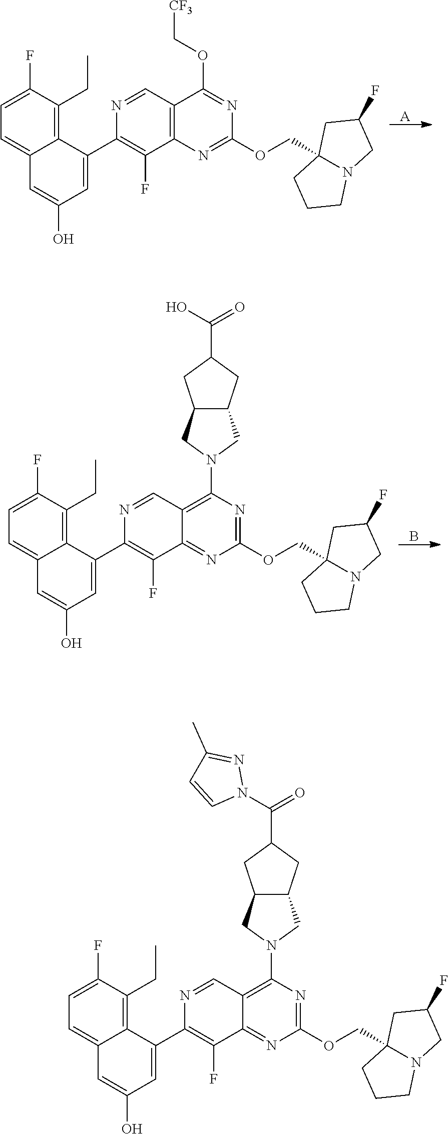

Step A. (3aS,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), (3aS,6aS)-octahydrocyclopenta[c]pyrrole-5-carboxylic acid (116 mg, 1.5 equiv, HCl) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 4 hours. The mixture was filtered and purified by prep-HPLC [column: Phenomenex luna C18 150×40 mm×15 μm; mobile phase: water(FA)-ACN; gradient: 15%-45% B over 15 minutes] to afford the title compound (70 mg, 27% yield) as white solid; LCMS (ESI, M+1): m/z=648.2.

Step B. ((3aS,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone: To a solution of (3aS,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (65.0 mg, 1.0 equiv) and 3-methyl-1H-pyrazole (12.4 mg, 1.5 equiv) in DMF (2 mL) were added HATU (57.0 mg, 1.5 equiv) and DIEA (65.0 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 46%-76% B over 20 minutes] to afford the title compound (11.1 mg, 14% yield) as off-white solid; ¹H NMR (400 MHz, CHLOROFORM-d) δ=9.16-8.99 (m, 1H), 8.18 (br d, J=2.0 Hz, 1H), 7.57-7.42 (m, 1H), 7.24-6.87 (m, 3H), 6.36-6.23 (m, 1H), 5.45-5.18 (m, 1H), 4.81-3.76 (m, 6H), 3.71-2.90 (m, 7H), 2.51-2.40 (m, 2H), 2.35 (s, 3H), 2.28-2.13 (m, 5H), 2.06-1.86 (m, 5H), 0.82 (q, J=7.6 Hz, 3H); LCMS (ESI, M+1): m/z=712.4.

### Example 116

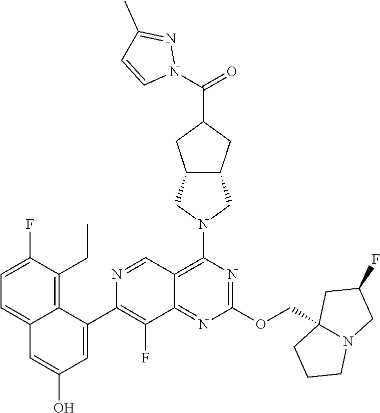 [SMILES: CCc1c(F)ccc2cc(O)cc(-c3ncc4c(N5C[C@H]6CC(C(=O)n7ccc(C)n7)C[C@H]6C5)nc(OC[C@@]56CCCN5C[C@H](F)C6)nc4c3F)c12]

### ((3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone

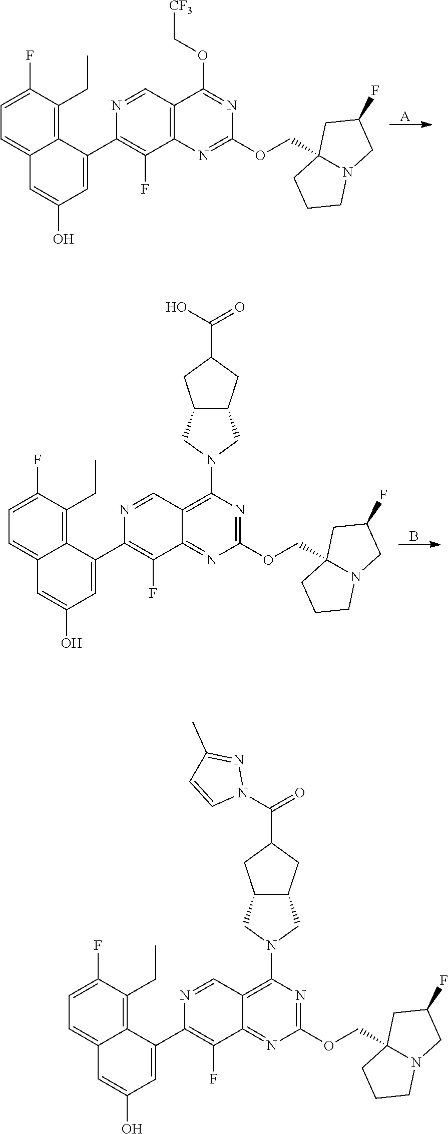

Step A. (3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), (3aR,6aS)-octahydrocyclopenta[c]pyrrole-5-carboxylic acid (116 mg, 1.5 equiv, HCl) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 4 hours. The mixture was filtered and purified by prep-HPLC [column: Phenomenex luna C18 150×40 mm×15 μm; mobile phase: water(FA)-ACN; gradient: 15%-45% B over 15 minutes] to afford the title compound (110 mg, 42% yield) as white solid; LCMS (ESI, M+1): m/z=648.2.

Step B. ((3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone: To a solution of (3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (105 mg, 1.0 equiv) and 3-methyl-1H-pyrazole (20 mg, 1.5 equiv) in DMF (2 mL) were added HATU (92.5 mg, 1.5 equiv) and DIEA (105 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 45%-75% B over 20 minutes] to afford the title compound (38 mg, 32% yield) as off-white solid; ¹H NMR (400 MHz, CHLOROFORM-d) δ=9.15 (br d, J=10.8 Hz, 1H), 8.19-8.09 (m, 1H), 7.49 (br dd, J=6.0, 8.8 Hz, 1H), 7.19-7.06 (m, 2H), 6.99-6.87 (m, 1H), 6.27 (d, J=2.8 Hz, 1H), 5.41-5.15 (m, 1H), 4.32-4.08 (m, 5H), 4.00-3.83 (m, 2H), 3.33-3.12 (m, 3H), 3.04-2.84 (m, 3H), 2.50-2.37 (m, 3H), 2.34 (s, 3H), 2.27-2.10 (m, 4H), 2.05-1.89 (m, 5H), 0.81 (br t, J=7.2 Hz, 3H); LCMS (ESI, M+1): m/z=712.4.

### Example 117

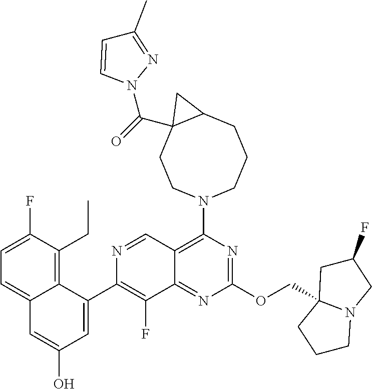 [SMILES: CCc1c(F)ccc2cc(O)cc(-c3ncc4c(N5CCCC6CC6(C(=O)n6ccc(C)n6)CC5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[6.1.0]nonan-1-yl)(3-methyl-1H-pyrazol-1-yl)methanone

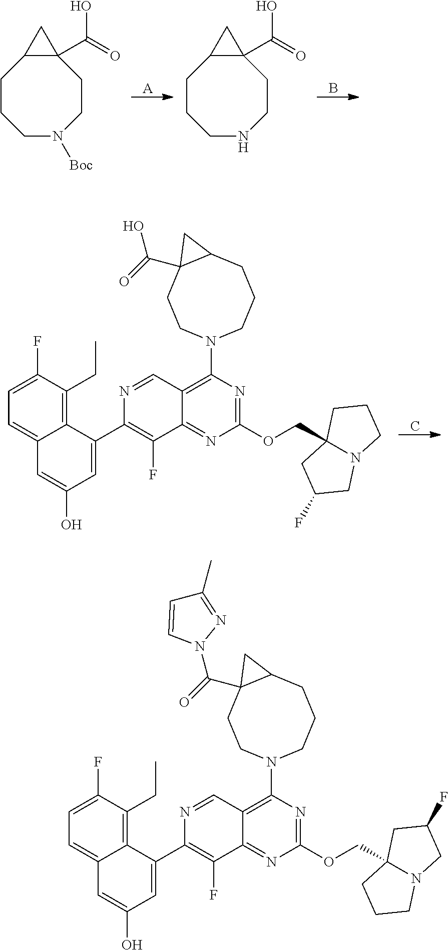

Step A. 4-azabicyclo[6.1.0]nonane-1-carboxylic acid: A solution of 4-(tert-butoxycarbonyl)-4-azabicyclo[6.1.0]nonane-1-carboxylic acid (300 mg, 1.0 equiv) in HCl·MeOH (2 M, 4 mL, 7.18 equiv) was stirred at 25 °C for 2 hours. The mixture was concentrated, dissolved with MeOH (4 mL), adjusted to pH=7 with NaHCO₃solid, filtered and concentrated to afford the title compound (150 mg, crude) as light brown solid.

Step B. 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[6.1.0]nonane-1-carboxylic acid: To a solution of 4-azabicyclo[6.1.0]nonane-1-carboxylic acid (114 mg, 2.0 equiv) in DMF (1 mL) were added DIEA (218 mg, 5.0 equiv) and 4 Å molecular sieve (100 mg) slowly at 25 °C. The reaction was stirred at 25 °C for 30 minutes. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (200 mg, 1.0 equiv) and K₃PO₄ (215 mg, 3.0 equiv) were added at 25 °C slowly. The reaction was stirred at 60 °C for 1 hour. The mixture was filtered and purified by reversed phase flash [Waters Xbridge 150×25 mm×10 μm; A: water(HCOOH), B: ACN, B %: 20%-50% over 1 minute] to afford the title compound (130 mg, 51% yield, HCOOH salt) as white solid; LCMS (ESI, M+1): m/z=662.3.

Step C. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[6.1.0]nonan-1-yl)(3-methyl-1H-pyrazol-1-yl)methanone: To a solution of 4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-azabicyclo[6.1.0]nonane-1-carboxylic acid (50 mg, 1.0 equiv, HCOOH) and 3-methyl-1H-pyrazole (17.4 mg, 3.0 equiv) in DMF (0.5 mL) were added HATU (80.6 mg, 3.0 equiv) and DIEA (54.8 mg, 6.0 equiv). The reaction was stirred at 25 °C for 1 hour. The mixture was filtered and purified by reversed phase flash chromatography [Waters Xbridge 150×25 mm×5 μm; A: water (NH₄HCO₃), B: ACN, B %: 56%-86% over 9 minutes] to afford the title compound (18.8 mg, 36% yield) as yellow solid; ¹H NMR (400 MHz, CHLOROFORM-d) δ=8.89 (br d, J=2.0 Hz, 1H), 8.25-8.10 (m, 1H), 7.62-7.43 (m, 1H), 7.20-7.12 (m, 2H), 7.11-6.95 (m, 1H), 6.25-6.19 (m, 1H), 5.47-5.11 (m, 1H), 4.89-4.68 (m, 1H), 4.25 (br s, 2H), 3.78-3.54 (m, 1H), 3.53-3.37 (m, 2H), 3.36-3.17 (m, 3H), 3.15-3.05 (m, 1H), 3.03-2.93 (m, 1H), 2.63-2.40 (m, 2H), 2.38-2.29 (m, 4H), 2.28-2.19 (m, 2H), 2.18-2.08 (m, 2H), 2.02-1.92 (m, 3H), 1.92-1.86 (m, 2H), 1.76-1.67 (m, 1H), 1.66-1.60 (m, 1H), 0.93-0.82 (m, 2H), 0.82-0.75 (m, 3H); LCMS (ESI, M+1): m/z=726.3.

### Example 118

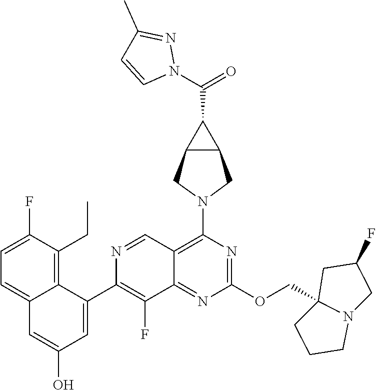 [SMILES: CCc1c(F)ccc2cc(O)cc(-c3ncc4c(N5C[C@@H]6[C@H](C5)[C@H]6C(=O)n5ccc(C)n5)nc(OC[C@@]56CCCN5C[C@H](F)C6)nc4c3F)c12]

### ((1R,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl)(3-methyl-1H-pyrazol-1-yl)methanone

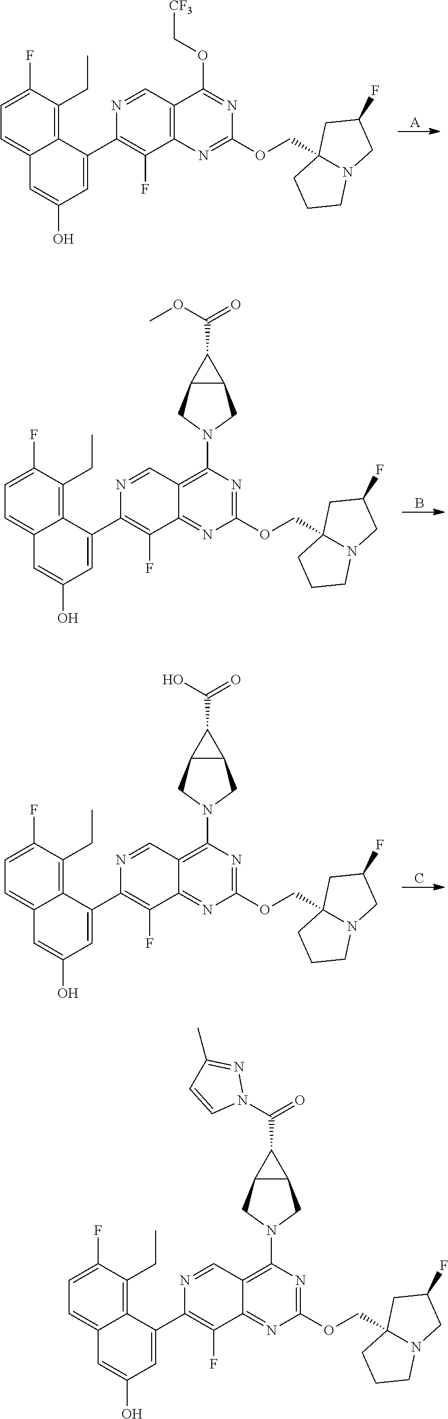

Step A. methyl (1R,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexane-6-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), methyl (1R,5S,6r)-3-azabicyclo[3.1.0]hexane-6-carboxylate (108 mg, 1.5 equiv, HCl) and 4 Å molecular sieve (50 mg) in DMF (4 mL) was added K₃PO₄ (430 mg, 5.0 equiv). The reaction was degassed and purged with nitrogen 3 times. The reaction was stirred at 60 °C for 1 hour. The mixture was quenched with H₂O (20 mL) and extracted with ethyl acetate (20 mL×3). The combined organic layers were washed with brine (20 mL×2), dried over anhydrous sodium sulfate and concentrated under reduced pressure to afford the title compound (220 mg, 86% yield) as yellow oil; LCMS (ESI, M+1): m/z=634.3.

Step B. (1R,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexane-6-carboxylic acid: To a solution of methyl (1R,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexane-6-carboxylate (215 mg, 1.0 equiv) in DMSO (2 mL) was added LiOH·H₂O (2 M, 1.7 ml, 10 equiv). The reaction was degassed and purged with nitrogen for 3 times. The reaction was stirred at 25 °C for 2 hours. The mixture was filtered and purified by prep-HPLC [column: Welch Xtimate C18 150×25 mm×5 μm; mobile phase: water(FA)-ACN; gradient: 13%-43% B over 15 minutes] to afford the title compound (65.0 mg, 31% yield) as white solid; LCMS (ESI, M+1): m/z=620.3.

Step C. ((1R,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl)(3-methyl-1H-pyrazol-1-yl)methanone: To a solution of (1R,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexane-6-carboxylic acid (70.0 mg, 1.0 equiv) and 3-methyl-1H-pyrazole (13.9 mg, 1.5 equiv) in DMF (1 mL) were added HATU (64.4 mg, 1.5 equiv) and DIEA (73.0 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 40%-70% B over 20 minutes] to afford the title compound (31.5 mg, 41% yield) as white solid; ¹H NMR (400 MHz, CHLOROFORM-d) δ=9.14-8.96 (m, 1H), 8.16 (d, J=2.8 Hz, 1H), 7.49-7.42 (m, 1H), 7.19-7.04 (m, 2H), 7.03-6.88 (m, 1H), 6.31 (d, J=2.8 Hz, 1H), 5.38-5.16 (m, 1H), 4.52-4.33 (m, 2H), 4.30-4.17 (m, 2H), 4.17-3.97 (m, 2H), 3.32-3.11 (m, 3H), 3.03-2.93 (m, 2H), 2.63-2.53 (m, 2H), 2.52-2.43 (m, 1H), 2.32 (s, 3H), 2.30-2.24 (m, 1H), 2.21 (br s, 1H), 2.15-2.07 (m, 2H), 1.98-1.87 (m, 3H), 0.81 (br t, J=7.2 Hz, 3H); LCMS (ESI, M+1): m/z=684.5.

### Example 119

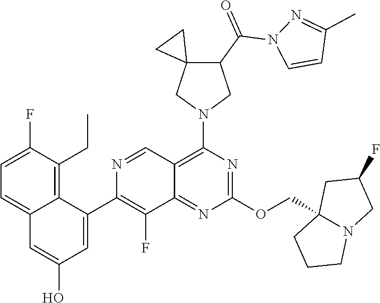 [SMILES: CCc1c(F)ccc2cc(O)cc(-c3ncc4c(N5CC(C(=O)n6ccc(C)n6)C6(CC6)C5)nc(OC[C@@]56CCCN5C[C@H](F)C6)nc4c3F)c12]

### (5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-azaspiro[2.4]heptan-7-yl)(3-methyl-1H-pyrazol-1-yl)methanone

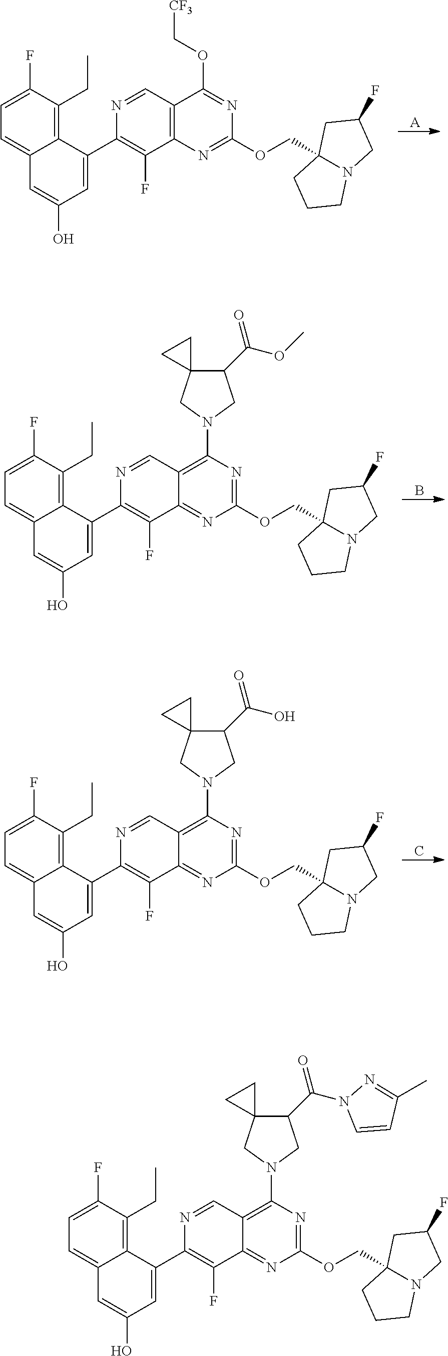

Step A. methyl 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-azaspiro[2.4]heptane-7-carboxylate: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), methyl 5-azaspiro[2.4]heptane-7-carboxylate (116 mg, 1.5 equiv, HCl) and 4 Å molecular sieve (50 mg) in DMF (4 mL) was added K₃PO₄ (430 mg, 5.0 equiv). The reaction was degassed and purged with nitrogen for 3 times. The reaction was stirred at 60 °C for 1 hour. The mixture was quenched with H₂O (20 mL) and extracted with ethyl acetate (20 mL×3). The combined organic layers were washed with brine (20 mL×2), dried over anhydrous sodium sulfate and concentrated under reduced pressure to afford the title compound (230 mg, 88% yield) as yellow oil; LCMS (ESI, M+1): m/z=648.3.

Step B. 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-azaspiro[2.4]heptane-7-carboxylic acid: To a solution of methyl 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-azaspiro[2.4]heptane-7-carboxylate (225 mg, 1.0 equiv) in DMSO (2 mL) was added LiOH·H₂O (2 M, 1.74 ml, 10 equiv). The reaction was degassed and purged with nitrogen 3 times. The reaction was stirred at 25 °C for 2 hours. The mixture was filtered and purified by prep-HPLC [column: Welch Xtimate C18 150×25 mm×5 μm; mobile phase: water(FA)-ACN; gradient: 13%-43% B over 15 minutes] to afford the title compound (65.0 mg, 29% yield) as white solid; LCMS (ESI, M+1): m/z=634.3.

Step C. (5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-azaspiro[2.4]heptan-7-yl)(3-methyl-1H-pyrazol-1-yl)methanone: To a solution of 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-azaspiro[2.4]heptane-7-carboxylic acid (60.0 mg, 1.0 equiv) and 3-methyl-1H-pyrazole (11.7 mg, 1.5 equiv) in DMF (1 mL) were added HATU (54.0 mg, 1.5 equiv) and DIEA (61.2 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 45%-75% B over 20 minutes] to afford the title compound (11.2 mg, 16% yield) as white solid; ¹H NMR (400 MHz, CHLOROFORM-d) δ=9.09 (br d, J=16.0 Hz, 1H), 8.16 (d, J=2.8 Hz, 1H), 7.48 (dd, J=6.4, 8.4 Hz, 1H), 7.17-7.07 (m, 2H), 7.00-6.89 (m, 1H), 6.28 (d, J=2.4 Hz, 1H), 5.51-5.05 (m, 1H), 4.45-4.17 (m, 4H), 4.10-3.87 (m, 2H), 3.33-3.19 (m, 2H), 3.18-3.07 (m, 3H), 3.02-2.93 (m, 1H), 2.52-2.42 (m, 1H), 2.39-2.27 (m, 4H), 2.26-2.15 (m, 2H), 2.10 (br d, J=10.0 Hz, 2H), 2.03-1.79 (m, 5H), 0.80 (t, J=7.2 Hz, 3H); LCMS (ESI, M+1): m/z=698.5.

### Example 120

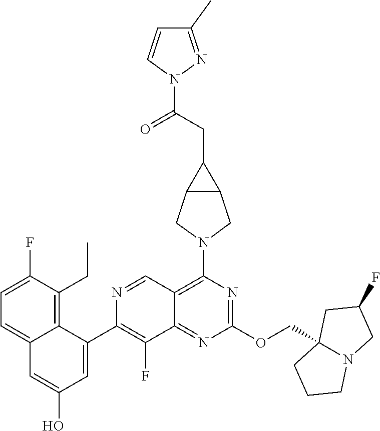 [SMILES: CCc1c(F)ccc2cc(O)cc(-c3ncc4c(N5CC6C(CC(=O)n7ccc(C)n7)C6C5)nc(OC[C@@]56CCCN5C[C@H](F)C6)nc4c3F)c12]

### 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl)-1-(3-methyl-1H-pyrazol-1-yl) ethan-1-one

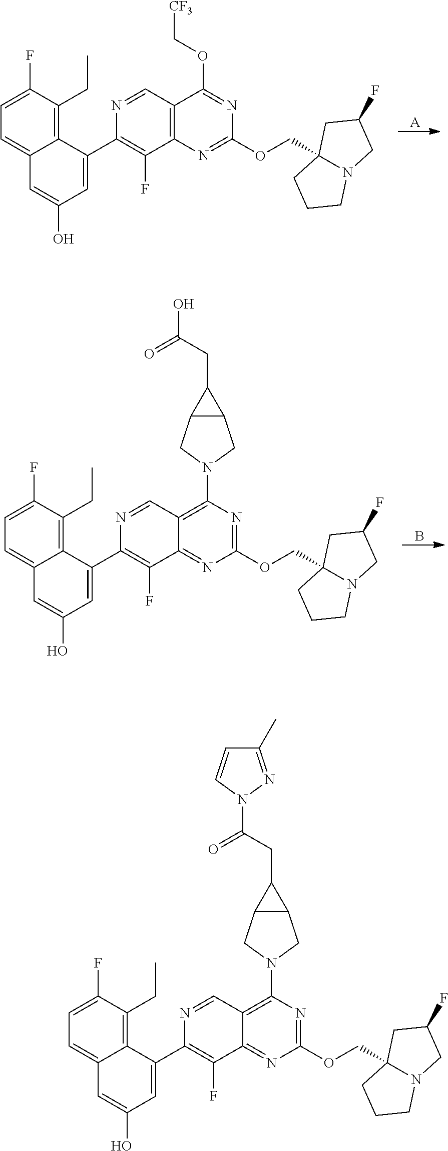

Step A. 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl) acetic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (240 mg, 1.0 equiv), 2-(3-azabicyclo[3.1.0]hexan-6-yl) acetic acid (85.8 mg, 1.5 equiv) and 4 Å molecular sieve (50 mg) in DMF (2 mL) was added K₃PO₄ (430 mg, 5.0 equiv). The reaction was degassed and purged with nitrogen for 3 times. The reaction was stirred at 60 °C for 1 hour. The mixture was filtered and purified by prep-HPLC [column: Phenomenex luna C18 150×40 mm×15 μm; mobile phase: water(FA)-ACN; gradient: 12%-42% B over 15 minutes] to afford the title compound (180 mg, 70% yield) as white solid; LCMS (ESI, M+1): m/z=634.4.

Step B. 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl)-1-(3-methyl-1H-pyrazol-1-yl) ethan-1-one: To a solution of 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.1.0]hexan-6-yl) acetic acid (175 mg, 1.0 equiv) and 3-methyl-1H-pyrazole (34 mg, 1.5 equiv) in DMF (2 mL) were added HATU (157 mg, 1.5 equiv) and DIEA (178 mg, 5.0 equiv). The reaction was degassed and purged with nitrogen for 3 times. The reaction was stirred at 25 °C for 1 hour. The mixture was filtered and purified by prep-HPLC [column: Waters Xbridge Prep OBD C18 150×40 mm×10 μm; mobile phase: water (NH₄HCO₃)-ACN; gradient: 43%-73% B over 20 minutes] to afford the title compound (31.4 mg, 15% yield) as white solid; ¹H NMR (400 MHz, CHLOROFORM-d) δ=9.22-9.05 (m, 1H), 8.17 (d, J=2.8 Hz, 1H), 7.51 (dd, J=6.0, 9.2 Hz, 1H), 7.20-7.10 (m, 2H), 7.05-6.94 (m, 1H), 6.32 (d, J=2.4 Hz, 1H), 5.38-5.13 (m, 1H), 4.51-4.40 (m, 2H), 4.31-4.17 (m, 3H), 3.77-3.62 (m, 1H), 3.31-3.11 (m, 3H), 3.01-2.92 (m, 1H), 2.55-2.42 (m, 1H), 2.33 (s, 3H), 2.29-2.19 (m, 2H), 2.18-2.09 (m, 2H), 1.99-1.77 (m, 5H), 1.30 (br dd, J=6.8, 19.2 Hz, 1H), 0.82 (br t, J=7.2 Hz, 5H); LCMS (ESI, M+1): m/z=698.4.

### Example 121

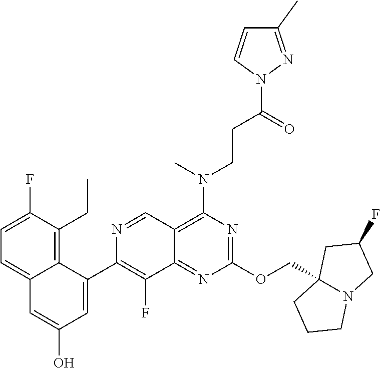 [SMILES: CCc1c(F)ccc2cc(O)cc(-c3ncc4c(N(C)CCC(=O)n5ccc(C)n5)nc(OC[C@@]56CCCN5C[C@H](F)C6)nc4c3F)c12]

### 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-1-(3-methyl-1H-pyrazol-1-yl) propan-1-one

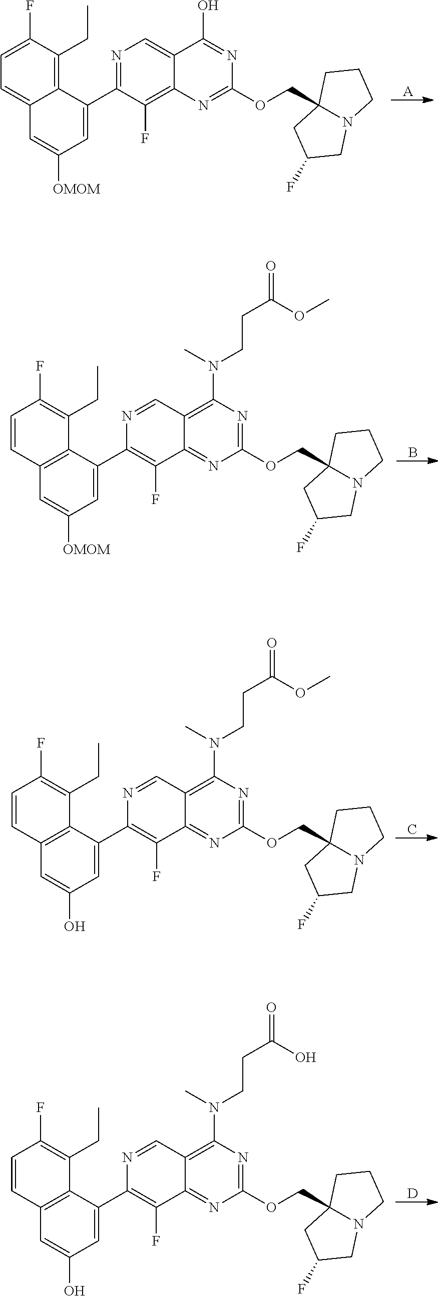

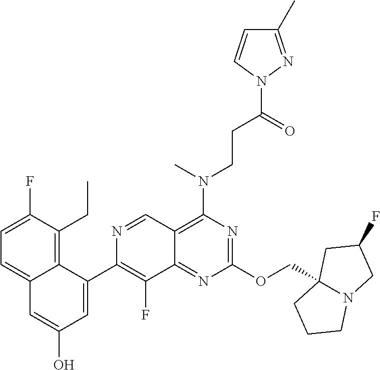 [SMILES: CCc1c(F)ccc2cc(O)cc(-c3ncc4c(N(C)CCC(=O)n5ccc(C)n5)nc(OC[C@@]56CCCN5C[C@H](F)C6)nc4c3F)c12]

Step A. methyl 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino) propanoate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (300 mg, 1.0 equiv) in DMF (3 mL) were added DIEA (210 mg, 3.0 equiv), PyBOP (619 mg, 2.2 equiv) and methyl 3-(methylamino) propanoate (95.0 mg, 1.5 equiv). The reaction was stirred at 25 °C for 1 hour. The mixture was diluted with water (10 mL) and extracted with ethyl acetate (10 mL×3). The combined organic layers were washed with brine (10 mL), dried over anhydrous sodium sulfate and concentrated to afford the title compound (452 mg, crude) as yellow solid; LCMS (ESI, M+1): m/z=654.3.

Step B. methyl 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino) propanoate: To a solution of methyl 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino) propanoate (452 mg, 1.0 equiv) in MeCN (2 mL) was added HCl·dioxane (2.0 mL, 2 M). The reaction was stirred at 25 °C for 0.5 hours. The mixture was concentrated, dissolved in MeOH (1 mL) and adjusted to pH=7 with NaHCO₃solid, filtered and concentrated to afford the title compound (370 mg, crude) as yellow solid; LCMS (ESI, M+1): m/z=610.3.

Step C. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propanoic acid: To a solution of methyl 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino) propanoate (370 mg, 1.0 equiv) in MeOH (2 mL) was added LiOH·H₂O (1.0 mL, 5.0 equiv, 2 M). The reaction was stirred at 25 °C for 0.5 hours. The mixture was filtered and purified by prep-HPLC [column: Phenomenex luna C18 150×40 mm×15 μm; mobile phase: [water(FA)-ACN]; gradient: 12%-42% B over 15 min] to afford the title compound (95.0 mg, 95% yield) as white solid; LCMS (ESI, M+1): m/z=596.3.

Step D. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-1-(3-methyl-1H-pyrazol-1-yl) propan-1-one: To a solution of 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propanoic acid (95.0 mg, 1.0 equiv) in DMF (2 mL) were added DIEA (103 mg, 5.0 equiv), 3-methyl-1H-pyrazole (20.0 mg, 1.5 equiv) and HATU (91.0 mg, 1.5 equiv). The reaction was stirred at 25 °C for 1 hour. The mixture was filtered and purified by prep-HPLC [column: Waters Xbridge Prep OBD C18 150×40 mm×10 μm; mobile phase: [water (NH₄HCO₃)-ACN]; gradient: 36%-66% B over 20 min] to afford the title compound (17.4 mg, 16% yield) as off-white solid; ¹H NMR (400 MHz, CHLOROFORM-d) δ=9.05 (d, J=10.8 Hz, 1H), 8.13 (t, J=2.4 Hz, 1H), 7.50-7.45 (m, 1H), 7.16-7.08 (m, 2H), 7.01-6.90 (m, 1H), 6.24 (t, J=2.4 Hz, 1H), 5.35-5.20 (m, 1H), 4.32-4.16 (m, 4H), 4.16-4.07 (m, 1H), 3.60-3.48 (m, 6H), 3.28 (br d, J=8.8 Hz, 2H), 3.19-3.10 (m, 2H), 3.00-2.96 (m, 1H), 2.49 (br dd, J=7.2, 14.8 Hz, 1H), 2.26 (d, J=4.4 Hz, 3H), 2.14-2.04 (m, 3H), 1.96 (br d, J=5.6 Hz, 1H), 0.81-0.78 (m, 3H); 1.25 (s, 3H); LCMS (ESI, M+1): m/z=660.4.

### Example 122

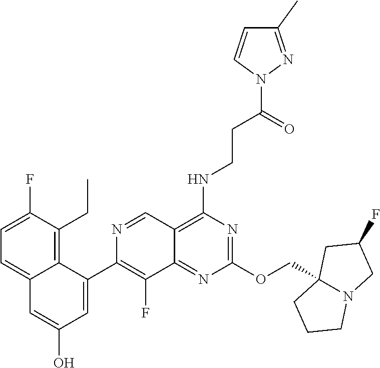 [SMILES: CCc1c(F)ccc2cc(O)cc(-c3ncc4c(NCCC(=O)n5ccc(C)n5)nc(OC[C@@]56CCCN5C[C@H](F)C6)nc4c3F)c12]

### 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) propan-1-one

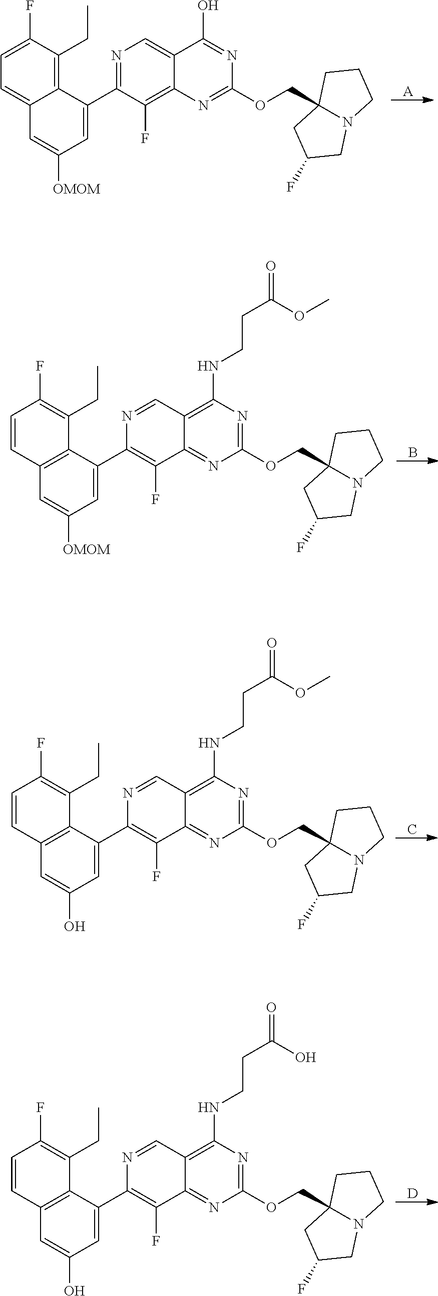

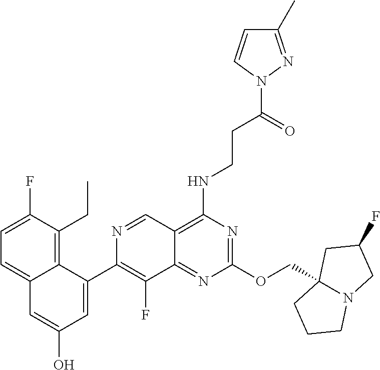 [SMILES: CCc1c(F)ccc2cc(O)cc(-c3ncc4c(NCCC(=O)n5ccc(C)n5)nc(OC[C@@]56CCCN5C[C@H](F)C6)nc4c3F)c12]

Step A. methyl 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino) propanoate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (300 mg, 1.0 equiv) in DMF (3 mL) were added DIEA (210 mg, 3.0 equiv), PyBOP (619 mg, 2.2 equiv) and methyl 3-aminopropanoate (113 mg, 1.5 equiv, HCl). The reaction was stirred at 25 °C for 1 hour. The mixture was diluted with water (10 mL) and extracted with ethyl acetate (10 mL×3). The combined organic layers were washed with brine (20 mL), dried over anhydrous sodium sulfate and concentrated to afford the title compound (336 mg, crude) as yellow solid; LCMS (ESI, M+1): m/z=640.2.

Step B. methyl 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino) propanoate: To a solution of methyl 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino) propanoate (336 mg, 1.0 equiv) in MeCN (2 mL) was added HCl·dioxane (2 mL, 2 M). The reaction was stirred at 25 °C for 0.5 hours. The mixture was concentrated, dissolved in MeOH (1 mL) and adjusted to pH=7 with NaHCO₃solid, filtered and concentrated to afford the title compound (310 mg, crude) as yellow solid; LCMS (ESI, M+1): m/z=596.3.

Step C. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)propanoic acid: To a solution of methyl 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino) propanoate (310 mg, 1.0 equiv) in MeOH (2 mL) was added LiOH·H₂O (1.0 mL, 5.0 equiv, 2 M). The reaction was stirred at 25 °C for 1 hour. The mixture was filtered and purified by prep-HPLC [column: Phenomenex luna C18 150×40 mm×15 μm; mobile phase: [water(FA)-ACN]; gradient: 12%-42% B over 15 min] to afford the title compound (65.0 mg, 99% yield) as white solid; LCMS (ESI, M+1): m/z=582.4.

Step D. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) propan-1-one: To a solution of 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)propanoic acid (65.0 mg, 1.0 equiv) in DMF (2 mL) were added DIEA (72.0 mg, 5.0 equiv), 3-methyl-1H-pyrazole (14.0 mg, 1.5 equiv) and HATU (64.0 mg, 1.5 equiv). The reaction was stirred at 25 °C for 1 hour. The mixture was filtered and purified by prep-HPLC [column: Waters Xbridge Prep OBD C18 150×40 mm×10 μm; mobile phase: [water (NH₄HCO₃)-ACN]; gradient: 33%-63% B over 20 min] to afford the title compound (14.8 mg, 20% yield) as white solid; ¹H NMR (400 MHz, DMSO-d₆) δ=9.93 (s, 1H), 9.26 (s, 1H), 9.12 (br t, J=5.2 Hz, 1H), 8.32 (d, J=2.4 Hz, 1H), 7.76 (dd, J=6.0, 9.2 Hz, 1H), 7.37-7.32 (m, 2H), 6.99 (d, J=2.0 Hz, 1H), 6.46 (d, J=2.8 Hz, 1H), 5.34-5.21 (m, 1H), 4.14-4.12 (m, 1H), 4.07-4.03 (m, 1H), 3.94-3.91 (m, 2H), 3.55-3.52 (m, 2H), 3.08 (br d, J=10.0 Hz, 2H), 3.01 (s, 1H), 2.85-2.81 (m, 1H), 2.37-2.31 (m, 1H), 2.23 (s, 3H), 2.14-2.12 (m, 1H), 2.06 (br s, 2H), 2.00 (br s, 1H), 1.84-1.76 (m, 3H), 0.70 (t, J=7.2 Hz, 3H); LCMS (ESI, M+1): m/z=646.3.

### Example 123

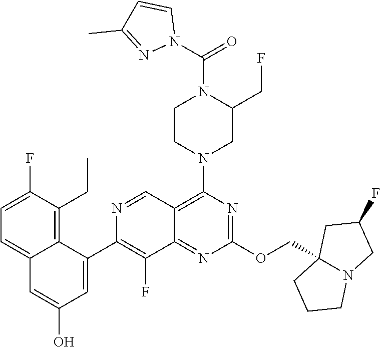 [SMILES: CCc1c(F)ccc2cc(O)cc(-c3ncc4c(N5CCN(C(=O)n6ccc(C)n6)C(CF)C5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(fluoromethyl)piperazin-1-yl)(3-methyl-1H-pyrazol-1-yl)methanone

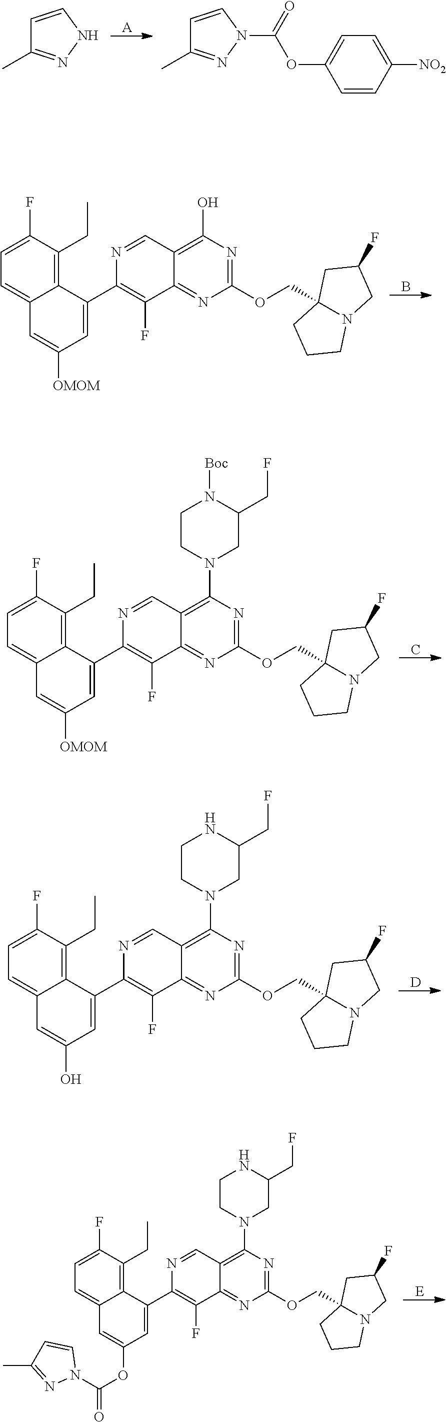

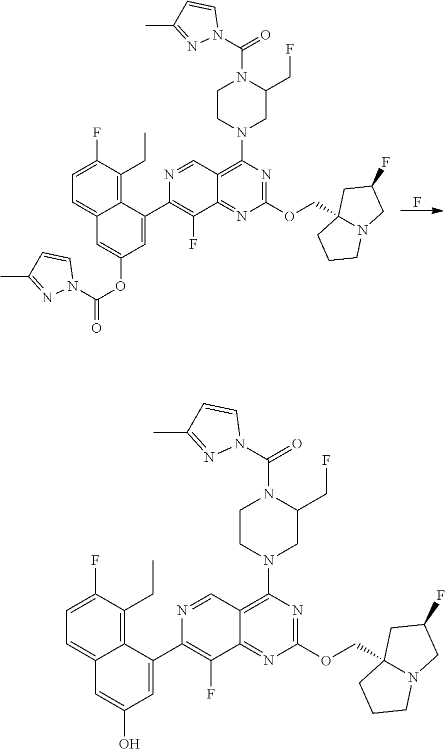

Step A. 4-nitrophenyl 3-methyl-1H-pyrazole-1-carboxylate: To a solution of 3-methyl-1H-pyrazole (600 mg, 1.0 equiv) and TEA (1.11 g, 1.5 equiv) in DCM (10 mL) was added 4-nitrophenyl carbonochloridate (1.62 g, 1.1 equiv) at 0 °C. The reaction was stirred at 20 °C for 2 hours. The mixture was diluted with water (10 mL) and extracted with DCM (2×10 mL). The combined organic layers were concentrated to afford the title compound (1.90 g, 95% yield) as yellow solid; LCMS (ESI, M+1): m/z=248.1.

Step B. tert-butyl 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(fluoromethyl)piperazine-1-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (350 mg, 1.0 equiv), tert-butyl 2-(fluoromethyl)piperazine-1-carboxylate (179 mg, 1.3 equiv) and TEA (191 mg, 3.0 equiv) in DMSO (3 mL) was added benzotriazol-1-yloxyl-tris-(pyrrolidino)-phosphonium hexafluorophosphate (493 mg, 1.5 equiv). The reaction was stirred at 25 °C for 0.5 hours. The mixture was filtered and purified by reversed phase flash [C18, 0.1% formic acid] to afford the title compound (420 mg, 86% yield) as yellow oil; LCMS (ESI, M+1): m/z=755.4.

Step C. 5-ethyl-6-fluoro-4-(8-fluoro-4-(3-(fluoromethyl)piperazin-1-yl)-2-(((2R,7aS)-2-fluorotetrahydro-1H-pyrrolizin-7a(5H)-yl)methoxy)pyrido[4,3-d]pyrimidin-7-yl)naphthalen-2-ol: To a solution of tert-butyl 4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(fluoromethyl)piperazine-1-carboxylate (415 mg, 1.0 equiv) in DCM (1 mL) was added TFA (3.07 g, 49 equiv) at 0 °C. The reaction was stirred at 0 °C for 2 hours. The mixture was diluted with DCM (8 mL). The mixture was adjusted to pH=7 with saturated NaHCO₃ (8 mL). The organic layer was dried over anhydrous sodium sulfate and concentrated to afford the title compound (330 mg, 69% yield) as yellow solid; LCMS (ESI, M+1): m/z=611.3.

Step D. 5-ethyl-6-fluoro-4-(8-fluoro-4-(3-(fluoromethyl)piperazin-1-yl)-2-(((2R,7aS)-2-fluorotetrahydro-1H-pyrrolizin-7a(5H)-yl)methoxy)pyrido[4,3-d]pyrimidin-7-yl)naphthalen-2-yl 3-methyl-1H-pyrazole-1-carboxylate: To a solution of 5-ethyl-6-fluoro-4-(8-fluoro-4-(3-(fluoromethyl)piperazin-1-yl)-2-(((2R,7aS)-2-fluorotetrahydro-1H-pyrrolizin-7a(5H)-yl)methoxy)pyrido[4,3-d]pyrimidin-7-yl)naphthalen-2-ol (320 mg, 1.0 equiv) and DIEA (203 mg, 3.0 equiv) in ACN (2 mL) was added 4-nitrophenyl 3-methyl-1H-pyrazole-1-carboxylate (155 mg, 1.2 equiv). The reaction was stirred at 0 °C for 10 minutes. The mixture was filtered and purified by prep-HPLC [column: Waters Xbridge 150×25 mm×5 μm; A: water (NH₄HCO₃), B: ACN, B %: 38%-58% over 10 minutes] to afford the title compound (100 mg, 23% yield) as yellow solid; LCMS (ESI, M+1): m/z=719.3.

Step E. 5-ethyl-6-fluoro-4-(8-fluoro-4-(3-(fluoromethyl)-4-(3-methyl-1H-pyrazole-1-carbonyl)piperazin-1-yl)-2-(((2R,7aS)-2-fluorotetrahydro-1H-pyrrolizin-7a(5H)-yl)methoxy)pyrido[4,3-d]pyrimidin-7-yl)naphthalen-2-yl 3-methyl-1H-pyrazole-1-carboxylate: To a solution of 5-ethyl-6-fluoro-4-(8-fluoro-4-(3-(fluoromethyl)piperazin-1-yl)-2-(((2R,7aS)-2-fluorotetrahydro-1H-pyrrolizin-7a(5H)-yl)methoxy)pyrido[4,3-d]pyrimidin-7-yl)naphthalen-2-yl 3-methyl-1H-pyrazole-1-carboxylate (100 mg, 1.0 equiv) and DIEA (53.9 mg, 3.0 equiv) in ACN (1.5 mL) was added 4-nitrophenyl 3-methyl-1H-pyrazole-1-carboxylate (68.8 mg, 2.0 equiv). The reaction was stirred at 40 °C for 10 hours. The mixture was filtered and purified by reversed phase flash [C18, 0.1% formic acid condition] to afford the title compound (40.0 mg, 27% yield) as yellow solid; LCMS (ESI, M+1): m/z=827.4.

Step F. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(fluoromethyl)piperazin-1-yl)(3-methyl-1H-pyrazol-1-yl)methanone: To a solution of 5-ethyl-6-fluoro-4-(8-fluoro-4-(3-(fluoromethyl)-4-(3-methyl-1H-pyrazole-1-carbonyl)piperazin-1-yl)-2-(((2R,7aS)-2-fluorotetrahydro-1H-pyrrolizin-7a(5H)-yl)methoxy)pyrido[4,3-d]pyrimidin-7-yl)naphthalen-2-yl 3-methyl-1H-pyrazole-1-carboxylate (38.0 mg, 1.0 equiv) in ACN (2 mL) and H₂O (0.2 mL) was added NaHCO₃ (38.6 mg, 10 equiv). The reaction was stirred at 20 °C for 12 hours. The mixture was diluted with water (5 mL) and extracted with ethyl acetate (2×6 mL). The combined organic layers were concentrated and purified by prep-HPLC [column: Waters Xbridge 150×25 mm×5 μm; A: water (NH₄HCO₃), B: ACN, B %: 42%-62% over 10 minutes] to afford the title compound (7.86 mg, 24% yield) as white solid; ¹H NMR (400 MHz, DMSO-d₆) δ=10.10-9.84 (m, 1H), 9.19 (s, 1H), 8.15 (s, 1H), 7.77 (dd, J=6.0, 9.2 Hz, 1H), 7.47-7.28 (m, 2H), 7.10-6.92 (m, 1H), 6.35 (d, J=2.4 Hz, 1H), 5.40-5.18 (m, 1H), 5.12-4.64 (m, 3H), 4.60-4.25 (m, 3H), 4.21-4.03 (m, 2H), 4.02-3.76 (m, 3H), 3.17-2.97 (m, 3H), 2.88-2.77 (m, 1H), 2.41-2.30 (m, 1H), 2.26 (s, 3H), 2.21-1.94 (m, 4H), 1.90-1.69 (m, 3H), 0.72 (q, J=7.2 Hz, 3H); LCMS (ESI, M+1): m/z=719.4.

### Example 124

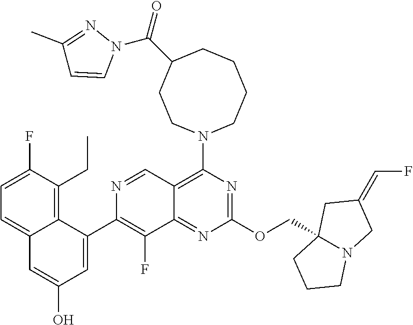 [SMILES: CCc1c(F)ccc2cc(O)cc(-c3ncc4c(N5CCCCC(C(=O)n6ccc(C)n6)CC5)nc(OC[C@@]56CCCN5C/C(=C\F)C6)nc4c3F)c12]

### (1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl) azocan-4-yl)(3-methyl-1H-pyrazol-1-yl)methanone

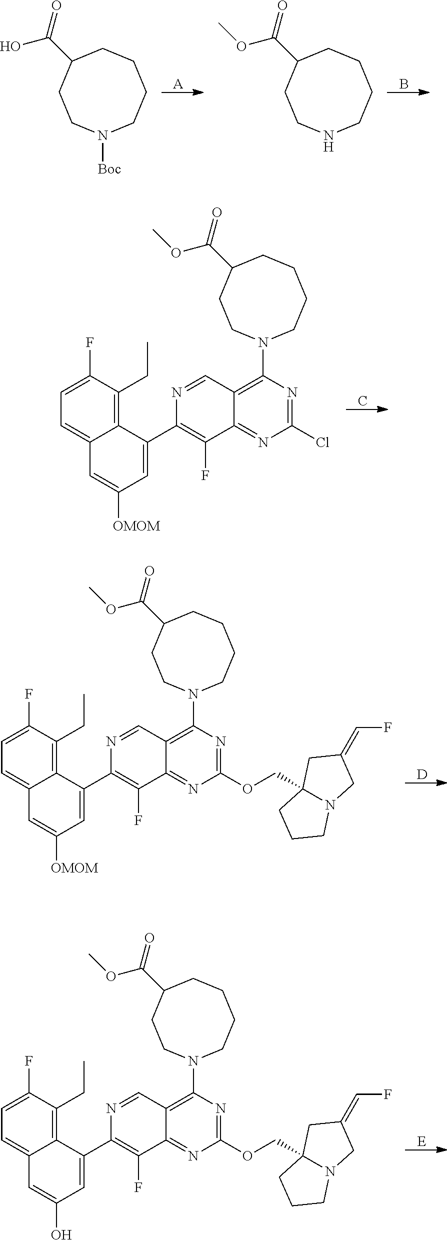

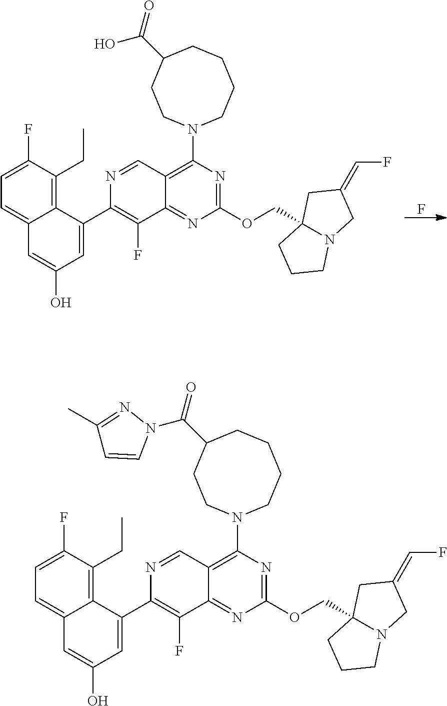

Step A. methyl azocane-4-carboxylate: To a solution of 1-(tert-butoxycarbonyl)azocane-4-carboxylic acid (200 mg, 1 equiv) in MeOH (2 mL) was added HCl·MeOH (2 M, 7.77 mL, 20 equiv). The reaction was stirred at 25 °C for 1 hour. The mixture was concentrated to afford the title compound (160 mg, 89% yield, HCl salt) as colorless oil.

Step B. methyl 1-(2-chloro-7-(8-ethyl-7-fluoro-3-(methoxymethoxy)naphthalen-1-yl)-8-fluoropyrido[4,3-d]pyrimidin-4-yl)azocane-4-carboxylate: To a solution of 2,4-dichloro-7-(8-ethyl-7-fluoro-3-(methoxymethoxy)naphthalen-1-yl)-8-fluoropyrido[4,3-d]pyrimidine (400 mg, 1 equiv) and TEA (449 mg, 5 equiv) in DCM (8 mL) was added methyl azocane-4-carboxylate (161 mg, 0.87 equiv, HCl salt) at −40 °C. The reaction was stirred at −40 °C for 1 hour. The mixture was diluted with water (20 mL) and extracted with DCM (2×20 mL). The organic layers were dried over anhydrous sodium sulfate, concentrated and purified by silica gel chromatography (petroleum ether/ethyl acetate 20/1 to 2/1) to afford the title compound (380 mg, 73% yield) as yellow solid; ¹H NMR (400 MHz, CHLOROFORM-d₃) δ 9.21 (s, 1H), 7.70 (dd, J=6.0, 9.2 Hz, 1H), 7.58-7.51 (m, 1H), 7.29-7.24 (m, 1H), 7.21 (dd, J=2.8, 11.6 Hz, 1H), 5.35-5.26 (m, 2H), 4.30-3.97 (m, 4H), 3.70 (d, J=2.8 Hz, 3H), 3.52 (s, 3H), 2.73-2.60 (m, 1H), 2.58-2.37 (m, 2H), 2.28-2.09 (m, 3H), 2.06-1.96 (m, 2H), 1.91-1.75 (m, 3H), 0.85 (q, J=7.2 Hz, 3H).

Step C. methyl 1-(7-(8-ethyl-7-fluoro-3-(methoxymethoxy)naphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)azocane-4-carboxylate: To a solution of (S,Z)-(2-(fluoromethylene)tetrahydro-1H-pyrrolizin-7a(5H)-yl)methanol (52.7 mg, 1.2 equiv) in THF (3 mL) was added NaH (25.6 mg, 60% purity, 2.5 equiv) at 0 °C. After stirring at 0 °C for 10 minutes, methyl 1-(2-chloro-7-(8-ethyl-7-fluoro-3-(methoxymethoxy)naphthalen-1-yl)-8-fluoropyrido[4,3-d]pyrimidin-4-yl)azocane-4-carboxylate (150 mg, 1 equiv) was added into the mixture. The reaction was stirred at 25 °C for 1 hour. The mixture was quenched with water (3 mL) and extracted with ethyl acetate (3×3 mL). The combined organic layers were dried over anhydrous sodium sulfate, concentrated and purified by reversed phase flash chromatography [C18, 0.1% formic acid condition] to afford the title compound (80 mg, 43% yield) as yellow solid; LCMS (ESI, M+1): m/z=720.4.

Step D. methyl 1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)azocane-4-carboxylate: To a solution of methyl 1-(7-(8-ethyl-7-fluoro-3-(methoxymethoxy)naphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)azocane-4-carboxylate (80 mg, 1 equiv) in ACN (0.2 mL) was added HCl·dioxane (2 M, 833 μL, 15 equiv). The reaction was stirred at 25 °C for 1 hour. The mixture was concentrated. The residue was basified with saturated NaHCO₃ solution (2 mL) and extracted with ethyl acetate (3×5 mL). The combined organic layers were dried over anhydrous sodium sulfate and concentrated to afford the title compound (70 mg, 84% yield) as yellow solid.

Step E. 1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)azocane-4-carboxylic acid: To a solution of methyl 1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)azocane-4-carboxylate (70 mg, 1 equiv) in ethanol (1.2 mL) was added a solution of NaOH (41.4 mg, 10 equiv) in H₂O (0.4 mL). The reaction was stirred at 25 °C for 1 hour. The mixture was filtered and purified by reversed phase flash chromatography [C18, 0.1% formic acid condition] to afford the title compound (65 mg, 90% yield) as yellow solid; ¹H NMR (400 MHz, DIMETHYL SULFOXIDE-d₆) δ 9.24-9.18 (m, 1H), 7.72-7.66 (m, 1H), 7.33-7.29 (m, 1H), 7.29-7.22 (m, 1H), 7.10-7.04 (m, 1H), 6.94-6.68 (m, 1H), 4.45-4.16 (m, 4H), 4.14-4.01 (m, 2H), 3.96-3.87 (m, 1H), 3.65-3.56 (m, 1H), 3.12-3.04 (m, 1H), 3.01-2.89 (m, 1H), 2.72-2.63 (m, 1H), 2.57-2.40 (m, 3H), 2.38-2.26 (m, 1H), 2.25-1.95 (m, 9H), 1.93-1.73 (m, 2H), 1.71-1.57 (m, 1H), 0.81 (dt, J=4.0, 7.2 Hz, 3H); LCMS (ESI, M+1): m/z=662.3.

Step F. (1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl) azocan-4-yl)(3-methyl-1H-pyrazol-1-yl)methanone: To a solution of 1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)azocane-4-carboxylic acid (55 mg, 1 equiv) and HATU (47.4 mg, 1.5 equiv) in DMF (0.55 mL) were added 3-methyl-1H-pyrazole (20.5 mg, 3 equiv) and DIEA (32.2 mg, 3 equiv). The reaction was stirred at 25 °C for 1 hour. The mixture was filtered. The filtrate was purified by prep-HPLC [Waters Xbridge 150×25 mm×5 μm; A: water (NH₄HCO₃); B: ACN, B %: 55%-85% over 9 min] to afford the title compound (6.33 mg, 10% yield) as white solid; ¹H NMR (400 MHz, DIMETHYL SULFOXIDE-d₆) δ 9.99 (s, 1H), 9.24-9.15 (m, 1H), 8.26-8.18 (m, 1H), 7.80-7.73 (m, 1H), 7.40-7.30 (m, 2H), 7.06-6.98 (m, 1H), 6.86-6.56 (m, 1H), 6.40 (t, J=2.8 Hz, 1H), 4.38-4.03 (m, 5H), 3.91-3.81 (m, 1H), 3.72-3.61 (m, 1H), 3.28-3.22 (m, 2H), 2.99-2.92 (m, 1H), 2.34-2.25 (m, 3H), 2.23-2.00 (m, 8H), 1.97-1.79 (m, 6H), 1.75-1.60 (m, 3H), 0.70 (td, J=7.2, 19.2 Hz, 3H); LCMS (ESI, M+1): m/z=726.3.

### Example 125

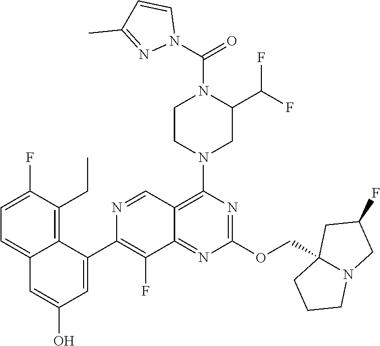 [SMILES: CCc1c(F)ccc2cc(O)cc(-c3ncc4c(N5CCN(C(=O)n6ccc(C)n6)C(C(F)F)C5)nc(OC[C@@]56CCCN5C[C@H](F)C6)nc4c3F)c12]

### (2-(difluoromethyl)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(3-methyl-1H-pyrazol-1-yl)methanone

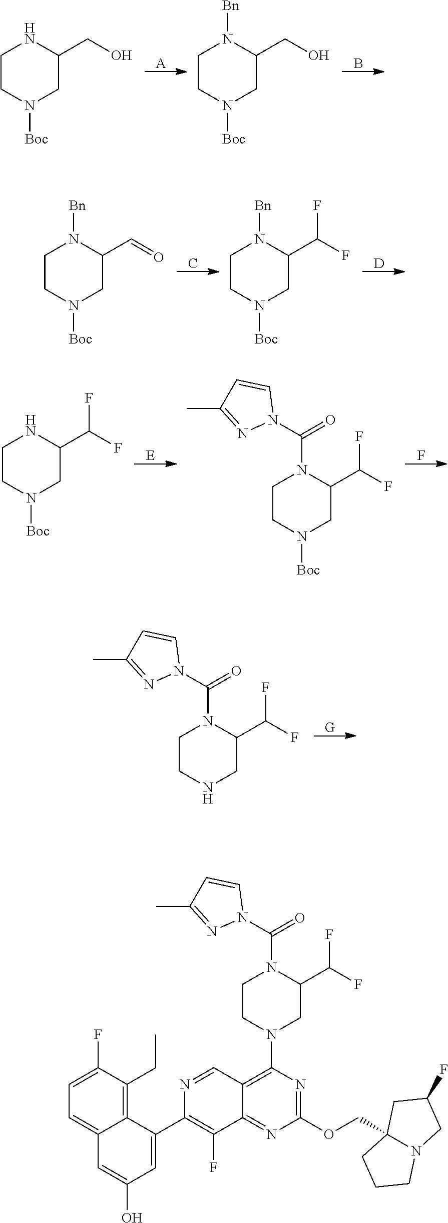

Step A. tert-butyl 4-benzyl-3-(hydroxymethyl)piperazine-1-carboxylate: To a solution of tert-butyl 3-(hydroxymethyl)piperazine-1-carboxylate (5.00 g, 1.0 equiv) and (bromomethyl)benzene (4.74 g, 1.2 equiv) in ACN (40 mL) was added TEA (4.68 g, 2.0 equiv). The reaction was stirred at 80 °C for 12 hours. The mixture was concentrated and purified by column chromatography (SiO₂, Petroleum ether/Ethyl acetate=1/0 to 2/1) to afford the title compound (5.60 g, 75% yield) as yellow oil; LCMS (ESI, M+1): m/z=307.3.

Step B. tert-butyl 4-benzyl-3-formylpiperazine-1-carboxylate: To a solution of (COCl)₂ (1.89 g, 1.2 equiv) in DCM (40 mL) was added DMSO (1.74 g, 1.8 equiv) at −70 °C. The reaction was stirred at −70 °C for 15 minutes. To the reaction was added tert-butyl 4-benzyl-3-(hydroxymethyl)piperazine-1-carboxylate (3.80 g, 1.0 equiv) in DCM (12 mL) slowly over 15 minutes. The reaction was stirred at −70 °C for 1 hour. To the mixture was added TEA (6.27 g, 5.0 equiv) at −70 °C. The reaction was warmed to 20 °C and stirred at 20 °C for 0.5 hours. The mixture was concentrated to afford the title compound (3.60 g, crude) as yellow oil; ¹H NMR (400 MHz, CHLOROFORM-d) δ=9.68 (d, J=2.4 Hz, 1H), 7.34-7.30 (m, 5H), 3.90 (br d, J=13.6 Hz, 1H), 3.70-3.54 (m, 4H), 3.32 (br d, J=8.4 Hz, 1H), 3.09-3.02 (m, 1H), 2.94 (ddd, J=3.2, 6.0, 11.6 Hz, 1H), 2.34-2.26 (m, 1H), 1.48 (s, 9H).

Step C. tert-butyl 4-benzyl-3-(difluoromethyl)piperazine-1-carboxylate: To a solution of tert-butyl 4-benzyl-3-formylpiperazine-1-carboxylate (3.30 g, 1.0 equiv) in DCM (40 mL) was added DAST (5.24 g, 3.0 equiv) at 0 °C. The reaction was stirred at 0 °C for 1 hour. The mixture was quenched with ice water (15 mL) and extracted with DCM (2×15 mL). The combined organic layers were concentrated and purified by column chromatography [SiO₂, Petroleum ether/Ethyl acetate=1/0 to 5/1] to afford the title compound (820 mg, 22% yield) as yellow oil; ¹H NMR (400 MHz, CHLOROFORM-d) δ=7.42-7.27 (m, 5H), 6.02 (dt, J=5.2, 55.4 Hz, 1H), 4.03-3.56 (m, 4H), 3.50-3.15 (m, 2H), 2.98-2.71 (m, 2H), 2.48 (br s, 1H), 1.47 (s, 9H); LCMS (ESI, M+1): m/z=327.2.

Step D. tert-butyl 3-(difluoromethyl)piperazine-1-carboxylate: To a solution of tert-butyl 4-benzyl-3-(difluoromethyl)piperazine-1-carboxylate (820 mg, 1.0 equiv) in MeOH (15 mL) and NH₃·MeOH (7 M, 0.2 mL, 0.5 equiv) was added Pd/C (300 mg, 10% purity, 0.1 equiv) under N₂. The suspension was degassed and purged with H₂ three times. The reaction was stirred under H₂ (50 psi) at 20 °C for 2 hours. The mixture was filtered and concentrated to afford the title compound (585 mg, 98% yield) as white gum.

Step E. tert-butyl 3-(difluoromethyl)-4-(3-methyl-1H-pyrazole-1-carbonyl)piperazine-1-carboxylate: To a solution of 3-methyl-1H-pyrazole (203 mg, 1.0 equiv) and DIEA (640 mg, 2.0 equiv) in DCM (5 mL) was added bis(trichloromethyl) carbonate (420 mg, 0.57 equiv) at 0 °C. The reaction was stirred at 0 °C for 1 hour. To the mixture was added tert-butyl 3-(difluoromethyl)piperazine-1-carboxylate (585 mg, 1.0 equiv). The reaction was stirred at 20 °C for 14 hours. The mixture was diluted with water (5 mL) and extracted with DCM (2×8 mL). The organic layers were concentrated and purified by column chromatography [SiO₂, Petroleum ether/Ethyl acetate=1/0 to 3/1] to afford the title compound (185 mg, 22% yield) as white solid; ¹H NMR (400 MHz, CHLOROFORM-d) δ=8.05 (d, J=2.4 Hz, 1H), 6.31-5.94 (m, 2H), 5.31-4.91 (m, 1H), 4.68-3.89 (m, 3H), 3.57-3.24 (m, 2H), 3.21-2.91 (m, 1H), 2.30 (s, 3H), 1.49 (s, 9H); LCMS (ESI, M−55): m/z=289.2.

Step F. (2-(difluoromethyl)piperazin-1-yl)(3-methyl-1H-pyrazol-1-yl)methanone: To a solution of tert-butyl 3-(difluoromethyl)-4-(3-methyl-1H-pyrazole-1-carbonyl)piperazine-1-carboxylate (175 mg, 1.0 equiv) in MeOH (0.5 mL) was added HCl·MeOH (2 M, 3 mL, 12 equiv). The reaction was stirred at 20 °C for 1 hour. The mixture was concentrated to afford the title compound (150 mg, crude, HCl) as white solid.

Step G. (2-(difluoromethyl)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(3-methyl-1H-pyrazol-1-yl)methanone: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (148 mg, 1.0 equiv), CsF (114 mg, 3.0 equiv) and 4 Å molecular sieve (50 mg) in DMF (2 mL) was added (2-(difluoromethyl)piperazin-1-yl)(3-methyl-1H-pyrazol-1-yl)methanone (70.0 mg, crude, HCl). The reaction was stirred at 20 °C for 2 hours. The mixture was diluted with H₂O (10 mL) and extracted with ethyl acetate (3×8 mL). The combined organic layers were dried over anhydrous sodium sulfate, concentrated and purified by prep-HPLC [column: Phenomenex luna C18 150×25 mm×10 μm; A: water(FA), B: ACN, B %: 24%-44% over 10 min] to afford the title compound (23.3 mg, 12% yield, 0.43 HCOOH) as white solid; ¹H NMR (400 MHz, DMSO-d6) δ=10.40-9.51 (s, 1H), 9.19 (s, 1H), 8.18 (d, J=2.8 Hz, 1H), 7.77 (dd, J=6.0, 9.2 Hz, 1H), 7.41-7.30 (m, 2H), 7.08-6.98 (m, 1H), 6.76-6.42 (m, 1H), 6.41-6.33 (m, 1H), 5.28 (br d, J=53.6 Hz, 1H), 4.78-4.63 (m, 1H), 4.60-4.29 (m, 2H), 4.22-4.06 (m, 2H), 4.04-3.93 (m, 1H), 3.91-3.71 (m, 2H), 3.17-2.98 (m, 4H), 2.89-2.78 (m, 1H), 2.44-2.31 (m, 1H), 2.26 (s, 3H), 2.22-1.93 (m, 4H), 1.91-1.69 (m, 3H), 0.82-0.61 (m, 3H); LCMS (ESI, M+1): m/z=737.3.

### Example 126

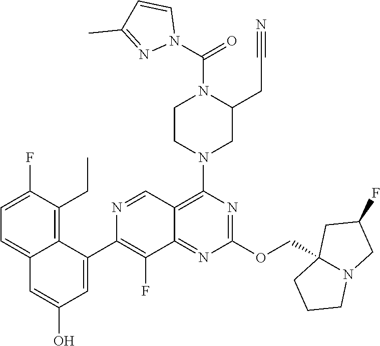 [SMILES: CCc1c(F)ccc2cc(O)cc(-c3ncc4c(N5CCN(C(=O)n6ccc(C)n6)C(CC#N)C5)nc(OC[C@@]56CCCN5C[C@H](F)C6)nc4c3F)c12]

### 2-(4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-(3-methyl-1H-pyrazole-1-carbonyl)piperazin-2-yl) acetonitrile

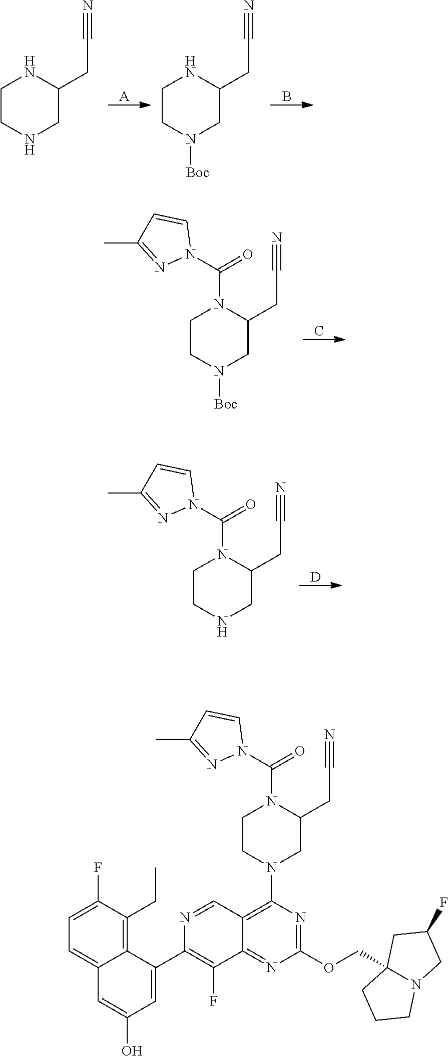

Step A. tert-butyl 3-(cyanomethyl)piperazine-1-carboxylate: To the solution of 2-(piperazin-2-yl) acetonitrile (3.00 g, 1.0 equiv) and TEA (12.1 g, 5.0 equiv) in MeOH (30 mL) was added Boc₂O (4.71 g, 0.9 equiv) at −30 °C. The reaction was stirred at 0 °C for 2 hours. The mixture was concentrated and purified by column chromatography [SiO₂, Petroleum ether/Ethyl acetate=1/0 to 1/1] to afford the title compound (2.00 g, 37% yield) as yellow oil; ¹H NMR (400 MHz, CHLOROFORM-d) δ=3.95 (br d, J=2.4 Hz, 1H), 3.88-3.77 (m, 1H), 3.09-2.90 (m, 3H), 2.87-2.62 (m, 2H), 2.55-2.37 (m, 2H), 1.75 (br s, 1H), 1.47 (s, 9H).

Step B. tert-butyl 3-(cyanomethyl)-4-(3-methyl-1H-pyrazole-1-carbonyl)piperazine-1-carboxylate: To a solution of 3-methyl-1H-pyrazole (364 mg, 1.0 equiv) and DIEA (1.15 g, 2.0 equiv) in DCM (10 mL) was added bis(trichloromethyl) carbonate (920 mg, 0.7 equiv) at 0 °C. The reaction was stirred at 0 °C for 1 hour. To the reaction was added tert-butyl 3-(cyanomethyl)piperazine-1-carboxylate (1.00 g, 1.0 equiv). The reaction was stirred at 20 °C for 12 hours. The mixture was quenched with water (5 mL) and extracted with DCM (2×8 mL). The combined organic layers were concentrated and purified by column chromatography [SiO₂, Petroleum ether/Ethyl acetate=1/0 to 3/1] and purified by prep-HPLC [column: Phenomenex luna C18 150×25 mm×10 μm; A: water(FA), B: ACN, B %: 40%-60% over 10 min] to afford the title compound (180 mg, 12% yield) as yellow oil; ¹H NMR (400 MHz, CHLOROFORM-d) δ=8.05 (d, J=2.0 Hz, 1H), 6.19 (d, J=2.4 Hz, 1H), 5.42-4.87 (m, 1H), 4.75-4.42 (m, 1H), 4.32-4.12 (m, 2H), 3.34-3.14 (m, 2H), 3.12-2.98 (m, 1H), 2.96-2.74 (m, 2H), 2.32 (s, 3H), 1.52 (s, 9H); LCMS (ESI, M+23): m/z=356.2.

Step C. 2-(1-(3-methyl-1H-pyrazole-1-carbonyl)piperazin-2-yl) acetonitrile: To a solution of tert-butyl 3-(cyanomethyl)-4-(3-methyl-1H-pyrazole-1-carbonyl)piperazine-1-carboxylate (90.0 mg, 1.0 equiv) in MeOH (0.5 mL) was added HCl·MeOH (2 M, 3 mL, 22 equiv). The reaction was stirred at 20 °C for 1 hour. The mixture was concentrated to afford the title compound (90.0 mg, crude, HCl) as white solid.

Step D. 2-(4-(7-(8-ethyl-7-fluoro-3-hydroxynaphthalen-1-yl)-8-fluoro-2-yl)-1-(3-methyl-1H-pyrazole-1-carbonyl)piperazin-2-yl) acetonitrile: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (143 mg, 1.0 equiv), CsF (110 mg, 3.0 equiv) and 4 Å molecular sieve (50 mg) in DMF (2 mL) was added 2-(1-(3-methyl-1H-pyrazole-1-carbonyl)piperazin-2-yl) acetonitrile (65.0 mg, crude, HCl). The reaction was stirred at 20 °C for 2 hours. The mixture was diluted with H₂O (10 mL) and extracted with ethyl acetate (3×8 mL). The combined organic layers were dried over anhydrous sodium sulfate, concentrated and purified by prep-HPLC [column: Phenomenex luna C18 150×25 mm×10 μm; A: water(FA), B: ACN, B %: 24%-44% over 10 min] to afford the title compound (16.5 mg, 9% yield, 0.3 HCOOH) as white solid; ¹H NMR (400 MHz, DMSO-d₆) δ=10.71-9.32 (s, 1H), 9.21 (d, J=2.0 Hz, 1H), 8.20-8.18 (m, 1H), 7.77 (dd, J=6.0, 9.2 Hz, 1H), 7.46-7.25 (m, 2H), 7.02 (dd, J=2.4, 8.0 Hz, 1H), 6.37 (d, J=2.8 Hz, 1H), 5.38-5.19 (m, 1H), 4.59-4.42 (m, 2H), 4.22-4.15 (m, 1H), 4.14-4.06 (m, 1H), 4.02-3.81 (m, 3H), 3.34-3.26 (m, 2H), 3.23-3.00 (m, 5H), 2.88-2.78 (m, 1H), 2.43-2.32 (m, 1H), 2.27 (s, 3H), 2.23-1.94 (m, 4H), 1.92-1.67 (m, 3H), 0.73 (q, J=7.2 Hz, 3H); LCMS (ESI, M+1): m/z=726.4.

### Example 127

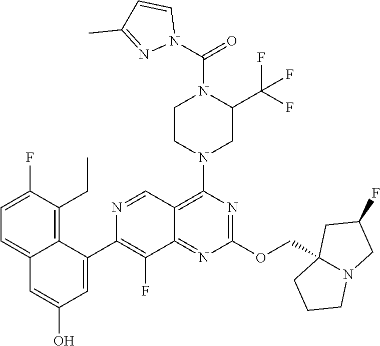 [SMILES: CCc1c(F)ccc2cc(O)cc(-c3ncc4c(N5CCN(C(=O)n6ccc(C)n6)C(C(F)(F)F)C5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(trifluoromethyl)piperazin-1-yl)(3-methyl-1H-pyrazol-1-yl)methanone

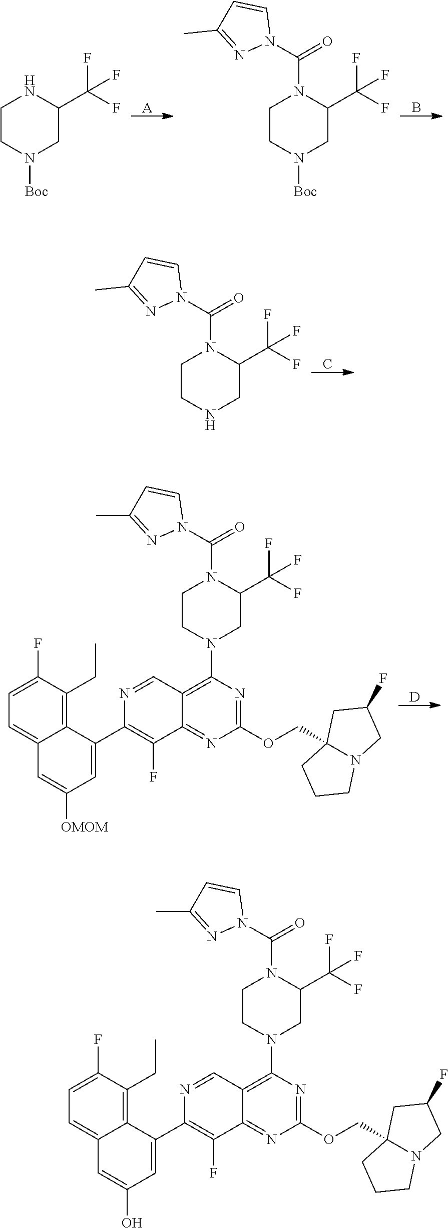

Step A. tert-butyl 4-(3-methyl-1H-pyrazole-1-carbonyl)-3-(trifluoromethyl)piperazine-1-carboxylate: To a solution of 3-methyl-1H-pyrazole (129 mg, 1.0 equiv) and DIEA (610 mg, 3.0 equiv) in DCM (6 mL) was added bis(trichloromethyl) carbonate (140 mg, 0.3 equiv) at 0 °C. The reaction was stirred at 0 °C for 1 hour. To the reaction was added tert-butyl 3-(trifluoromethyl)piperazine-1-carboxylate (400 mg, 1.0 equiv). The reaction was stirred at 20 °C for 14 hours. The mixture was quenched with water (5 mL) and extracted with DCM (2×8 mL). The organic layers were concentrated and purified by column chromatography [SiO₂, Petroleum ether/Ethyl acetate=1/0 to 4/1] and purified by prep-HPLC [column: Phenomenex luna C18 150×25 mm×10 μm; A: water(FA), B: ACN, B %: 54%-74% over 10 min] to afford the title compound (120 mg, 21% yield) as white solid; ¹H NMR (400 MHz, CHLOROFORM-d) δ=8.05 (d, J=2.8 Hz, 1H), 6.18 (d, J=2.8 Hz, 1H), 6.08-5.10 (m, 1H), 4.84-4.12 (m, 3H), 3.56-3.18 (m, 2H), 3.16-2.87 (m, 1H), 2.30 (s, 3H), 1.48 (s, 9H); LCMS (ESI, M−55): m/z=307.2.

Step B. (3-methyl-1H-pyrazol-1-yl)(2-(trifluoromethyl)piperazin-1-yl)methanone: To a solution of tert-butyl 4-(3-methyl-1H-pyrazole-1-carbonyl)-3-(trifluoromethyl)piperazine-1-carboxylate (100 mg, 1.0 equiv) in MeOH (0.5 mL) was added HCl·MeOH (2 M, 1 mL, 7.3 equiv). The reaction was stirred at 20 °C for 0.5 hours. The mixture was concentrated to afford the title compound (70.0 mg, crude, HCl) as white solid.

Step C (4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(trifluoromethyl)piperazin-1-yl)(3-methyl-1H-pyrazol-1-yl)methanone: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (74.0 mg, 1.0 equiv), benzotriazol-1-yloxyl-tris-(pyrrolidino)-phosphonium hexafluorophosphate (105 mg, 1.5 equiv) and TEA (81.0 mg, 6.0 equiv) in DMSO (1 mL) was added (3-methyl-1H-pyrazol-1-yl)(2-(trifluoromethyl)piperazin-1-yl)methanone (40.0 mg, crude, HCl). The reaction was stirred at 30 °C for 12 hours. The mixture was filtered and purified by purified by reversed phase flash [C18, 0.1% formic acid condition] to give the title compound (94.0 mg, 82% yield) as yellow oil; LCMS (ESI, M+1): m/z=799.3.

Step D (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(trifluoromethyl)piperazin-1-yl)(3-methyl-1H-pyrazol-1-yl)methanone: To a solution of (4-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-(trifluoromethyl)piperazin-1-yl)(3-methyl-1H-pyrazol-1-yl)methanone (90.0 mg, 1.0 equiv) in DCM (1 mL) was added TFA (3.07 g, 239 equiv). The reaction was stirred at 0 °C for 0.5 hours. The mixture was concentrated. The residue was diluted with water (1 mL). The mixture was adjusted to pH=8 with saturated NaHCO₃ (5 mL). The mixture was extracted with ethyl acetate (2×10 mL). The combined organic layers were dried over anhydrous sodium sulfate, concentrated and purified by prep-HPLC [column: Phenomenex luna C18 150×25 mm×10 μm; A: water(FA), B: ACN, B %: 22%-52% over 10 min] and reversed phase flash [C18, water/ACN] to afford the title compound (3.23 mg, 4% yield) as white solid; ¹H NMR (400 MHz, DMSO-d₆) δ=9.96 (br s, 1H), 9.18 (d, J=4.8 Hz, 1H), 8.24-8.16 (m, 1H), 7.77 (dd, J=6.0, 9.2 Hz, 1H), 7.42-7.28 (m, 2H), 7.12-6.96 (m, 1H), 6.39 (t, J=2.8 Hz, 1H), 5.39-5.15 (m, 1H), 4.98-4.74 (m, 1H), 4.71-4.40 (m, 2H), 4.22-4.06 (m, 3H), 4.00-3.72 (m, 2H), 3.19-2.93 (m, 4H), 2.88-2.78 (m, 1H), 2.41-2.31 (m, 1H), 2.27 (d, J=2.4 Hz, 3H), 2.20-1.98 (m, 4H), 1.90-1.73 (m, 3H), 0.71 (td, J=7.2, 11.2 Hz, 3H); LCMS (ESI, M+1): m/z=755.3.

### Example 128

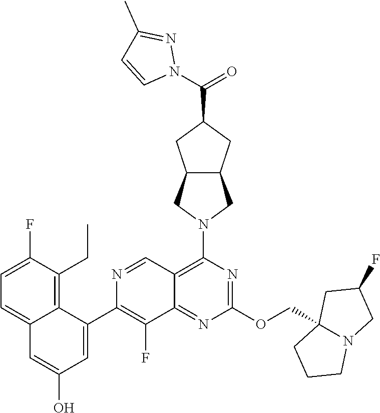 [SMILES: CCc1c(F)ccc2cc(O)cc(-c3ncc4c(N5C[C@H]6C[C@@H](C(=O)n7ccc(C)n7)C[C@H]6C5)nc(OC[C@@]56CCCN5C[C@H](F)C6)nc4c3F)c12]

### ((3aR,5S,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone

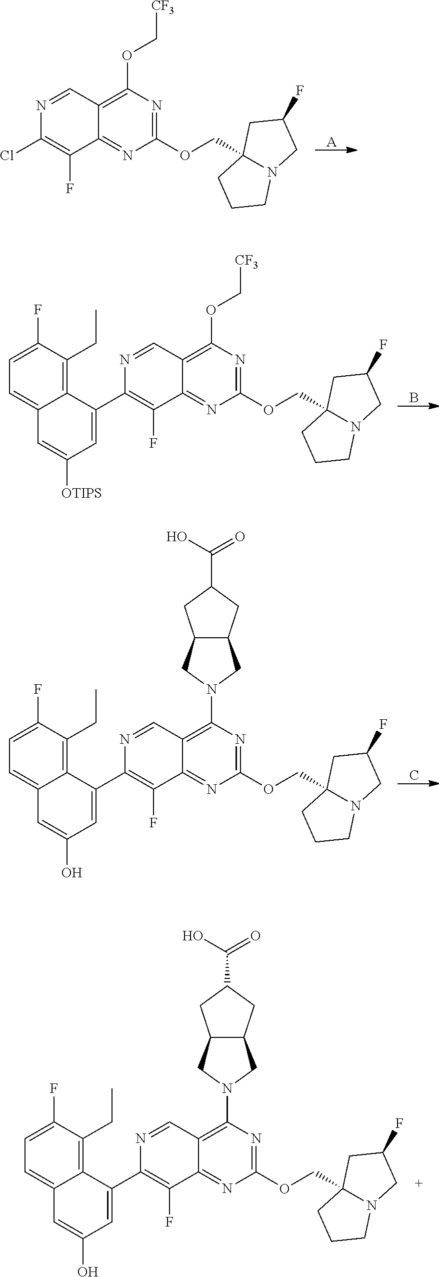

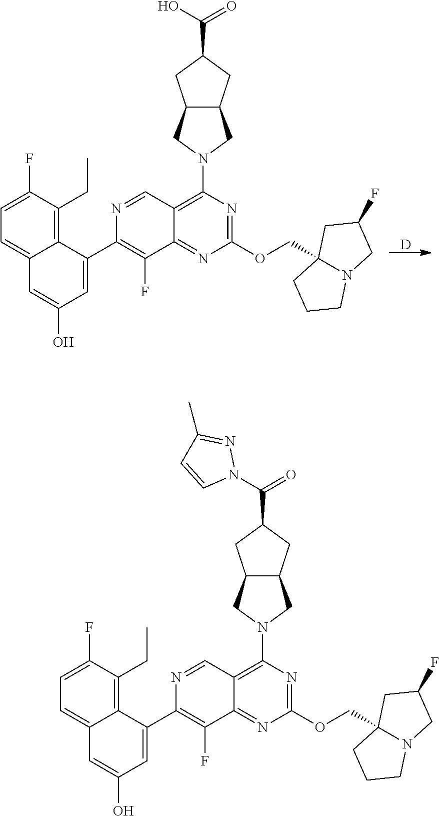

Step A. 7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (7.2 g, 1.0 equiv) and ((5-ethyl-6-fluoro-4-(4,4,5,5-tetramethyl-1,3,2-dioxaborolan-2-yl)naphthalen-2-yl)oxy)triisopropylsilane (11.6 g, 1.5 equiv) in methoxycyclopentane (71 mL) were added CataCXium A Pd G3 (1.2 g, 0.1 equiv) and Cs₂CO₃ (13.4 g, 2.5 equiv). The reaction was degassed and purged with nitrogen 3 times. The reaction was stirred at 100 °C for 3 hours. The mixture was diluted with water (100 mL) and extracted with ethyl acetate (2×60 mL). The combined organic layers were washed with brine (50 mL), dried over anhydrous sodium sulfate, concentrated and purified by reversed phase flash chromatography [C18, 0.1% formic acid condition] to afford the title compound (3.5 g, 26% yield) as yellow solid; LCMS (ESI, M+1): m/z=749.3.

Step B. (3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid: To a solution of 7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (500 mg, 1.0 equiv) and (3aR,6aS)-octahydrocyclopenta[c]pyrrole-5-carboxylic acid (124 mg, 1.2 equiv) in DMAC (5 mL) were added K₃PO₄ (425.18 mg, 3.0 equiv) and 4 Å molecular sieve (500 mg). The reaction was stirred at 60 °C for 1 hour. The mixture was filtered and purified by reversed phase flash chromatography [C18, 0.1% formic acid condition] to afford the title compound (410 mg, 44% yield) as yellow solid; LCMS (ESI, M+1): m/z=648.2.

Step C. (3aR,5S,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid: (3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (410 mg, 1.0 equiv) was purified by SFC separation [column: DAICEL CHIRALPAK IC, 250×30 mm, 10 μm; A: CO₂, B: ACN/i-PrOH (30%/70%)(0.1% NH₃H₂O), B %: 50%-50% B over 3.7 min] and reversed phase flash chromatography [C18, 0.1% formic acid condition] to afford two isomers.

Isomer 1:(3aR,5R,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (166 mg, 38% yield) as yellow solid; LCMS (ESI, M+1): m/z=648.3; SFC>99% ee, column: Chiralpak IC-3 50×4.6 mm I.D., 3 μm; mobile phase: 20% to 60% IPA+ACN (0.05% DEA) in CO₂, flow rate: 3 mL/min, detector: PDA, t_{R}: 1.668 min.

Isomer 2: (3aR,5S,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (32 mg, 6.8% yield) as yellow solid; LCMS (ESI, M+1): m/z=648.3; SFC=78% ee, column: Chiralpak IC-3 50×4.6 mm I.D., 3 μm; mobile phase: 20% to 60% IPA+ACN (0.05% DEA) in CO₂, flow rate: 3 mL/min, detector: PDA, t_{R}: 1.974 min.

Step D. ((3aR,5S,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone: To a solution of (3aR,5S,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (80 mg, 1.0 equiv) and 3-methyl-1H-pyrazole (50.7 mg, 5 equiv) in DMF (1 mL) were added HATU (93.9 mg, 2.0 equiv) and DIEA (79.8 mg, 6.0 equiv). The reaction was stirred at 25 °C for 1 hour. The mixture was filtered and purified by by prep-HPLC [Waters Xbridge 150×25 mm×5 μm; A: water (NH₄HCO₃), B: ACN, B %: 55%-85% over 9 min] to afford the title compound (5.03 mg, 4.2% yield) as white solid; ¹H NMR (400 MHz, CHLOROFORM-d) δ=9.11 (br d, J=18.8 Hz, 1H), 8.12 (d, J=2.8 Hz, 1H), 7.43 (m, 1H), 7.11 (t, J=9.2 Hz, 1H), 7.04 (dd, J=2.4, 6.0 Hz, 1H), 6.99-6.86 (m, 1H), 6.26 (d, J=2.4 Hz, 1H), 5.35-5.18 (m, 1H), 4.33-4.18 (m, 3H), 4.12-3.96 (m, 2H), 3.94-3.77 (m, 2H), 3.41-3.09 (m, 3H), 3.04-2.92 (m, 1H), 2.91-2.71 (m, 2H), 2.51-2.34 (m, 3H), 2.33 (s, 3H), 2.30-2.25 (m, 1H), 2.24-2.07 (m, 3H), 2.01-1.80 (m, 5H), 0.79 (t, J=7.2 Hz, 3H); LCMS (ESI, M+1): m/z=712.3.

### Example 129

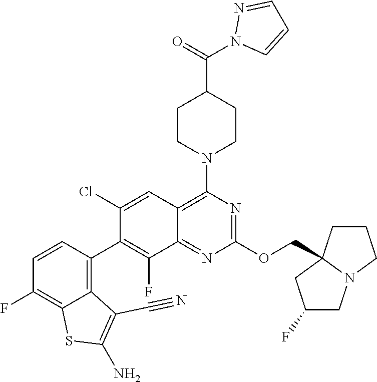 [SMILES: N#Cc1c(N)sc2c(F)ccc(-c3c(Cl)cc4c(N5CCC(C(=O)n6cccn6)CC5)nc(OC[C@@]56CCCN5C[C@H](F)C6)nc4c3F)c12]

### 4-(4-(4-(1H-pyrazole-1-carbonyl)piperidin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

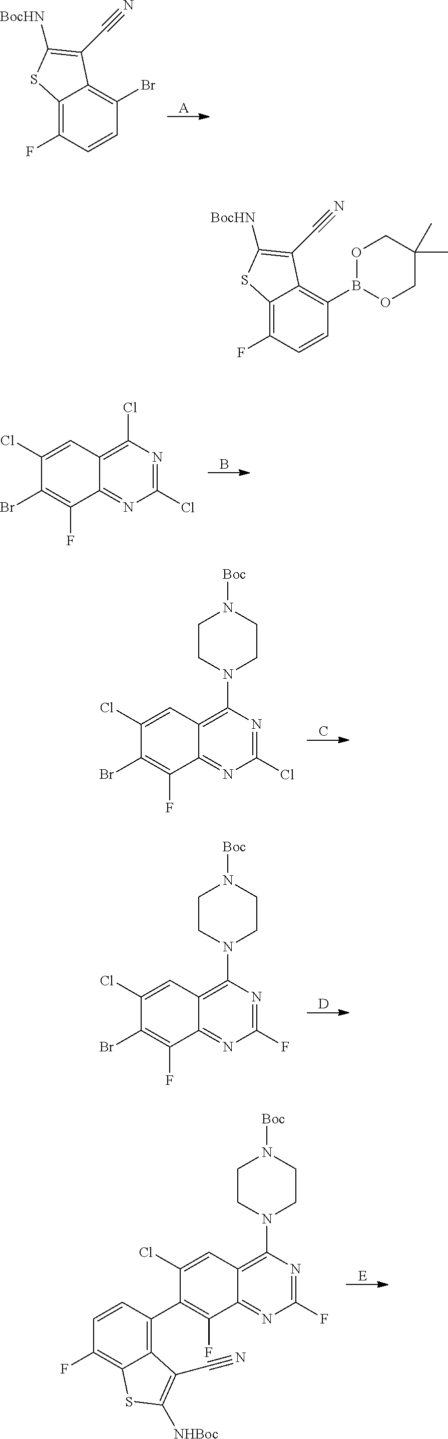

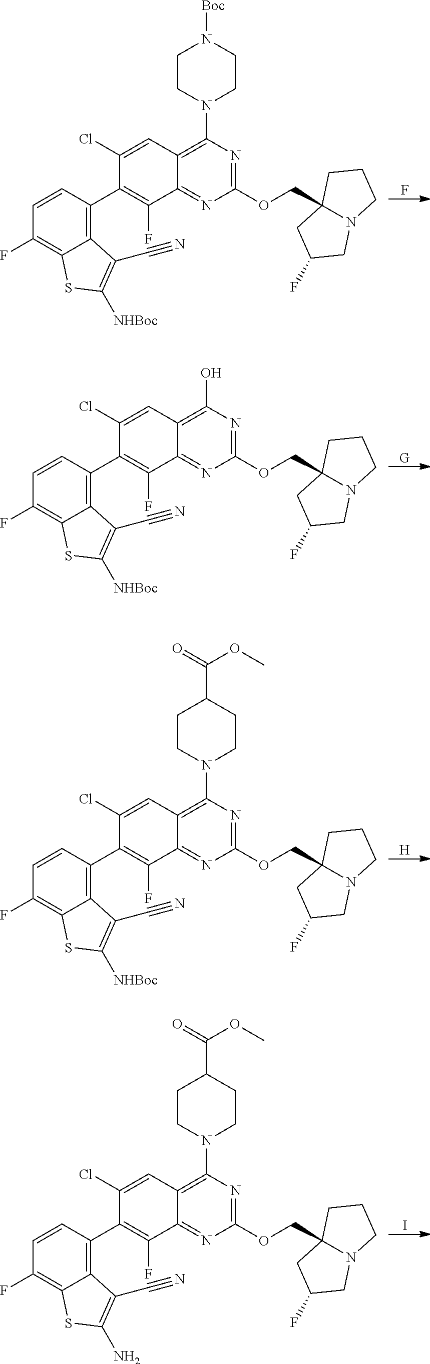

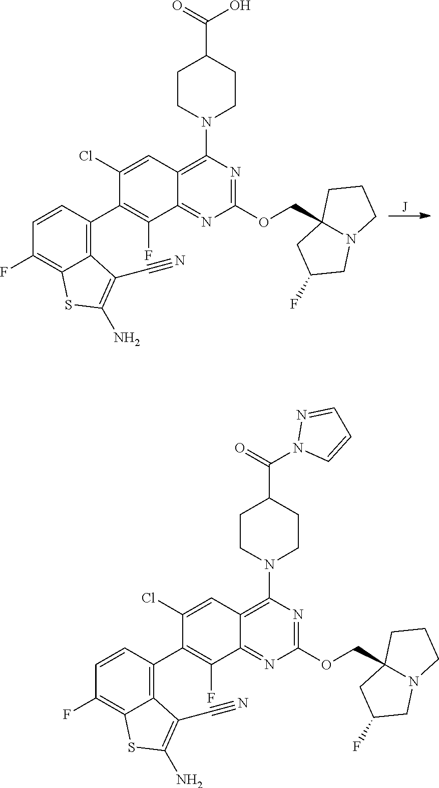

Step A. tert-butyl (3-cyano-4-(5,5-dimethyl-1,3,2-dioxaborinan-2-yl)-7-fluorobenzo[b]thiophen-2-yl)carbamate: To a mixture of tert-butyl (4-bromo-3-cyano-7-fluorobenzo[b]thiophen-2-yl)carbamate (250 g, 1.0 equiv) and 5,5,5′,5′-tetramethyl-2,2′-bi (1,3,2-dioxaborinane) (182 g, 1.2 equiv) in dioxane (2.5 L) were added potassium acetate (198 g, 3.0 equiv) and Pd(DPEphos)Cl₂ (46.3 g, 0.1 equiv). The reaction was degassed and purged with nitrogen for 3 times. The reaction was stirred at 90 °C for 2 hours. The mixture was quenched with water (5.0 L) and extracted by ethyl acetate (3×3.0 L). The combined organic layers were washed with brine (2.0 L), dried over anhydrous sodium sulfate, concentrated, and purified by column chromatography (SiO₂, Petroleum ether/Ethyl acetate=1/0 to 10/1) to afford the title compound (170 g, 43% yield) as yellow solid; ¹H NMR (400 MHz, DMSO-d₆) δ=11.58 (br s, 1H), 7.59 (dd, J=6.0, 8.0 Hz, 1H), 7.19 (dd, J=8.8, 9.6 Hz, 1H), 3.77 (s, 4H), 1.53 (s, 9H), 1.02 (s, 6H).

Step B. tert-butyl 4-(7-bromo-2,6-dichloro-8-fluoroquinazolin-4-yl)piperazine-1-carboxylate: To a solution of 7-bromo-2,4,6-trichloro-8-fluoroquinazoline (250 g, 1.0 equiv) and triethylamine (230 g, 3.0 equiv) in dichloromethane (4.0 L) was slowly added tert-butyl piperazine-1-carboxylate (134 g, 0.95 equiv) at −40 °C The reaction was stirred at −40 °C for 1 hour. The mixture was quenched with water (10.0 L) and extracted with dichloromethane (6.0 L×2). The combined organic layers were washed with saturated ammonium chloride (2.0 L×3) and brine (2.0 L×2), dried over anhydrous sodium sulfate, concentrated to afford the title compound (360 g, crude) as yellow solid; ¹H NMR (400 MHz, DMSO-d6) δ=8.09 (d, J=2.0 Hz, 1H), 3.92 (dd, J=4.8, 6.8 Hz, 4H), 3.55 (br s, 4H), 1.43 (s, 9H); LCMS (ESI, M+1): m/z=480.9.

Step C. tert-butyl 4-(7-bromo-6-chloro-2,8-difluoroquinazolin-4-yl)piperazine-1-carboxylate: To a mixture of tert-butyl 4-(7-bromo-2,6-dichloro-8-fluoroquinazolin-4-yl)piperazine-1-carboxylate (360 g, 1.0 equiv) and potassium fluoride (436 g, 10.0 equiv) in (methylsulfinyl) methane (3.0 L) was added 1,4,7,10,13,16-hexaoxacyclooctadecane (19.9 g, 0.1 equiv). The reaction was stirred at 120 °C for 4 hours. The mixture was diluted with water (10.0 L) and extracted with ethyl acetate (3.0 L×2). The combined organic layers were washed with brine (2.0 L×3), dried over anhydrous sodium sulfate, concentrated, and triturated with petroleum ether/ethyl acetate=5/1 (200 mL) at 20 °C for 30 minutes to afford the title compound (300 g, 76% yield) as yellow solid; LCMS (ESI, M+1): m/z=464.9.

Step D. tert-butyl 4-(7-(2-((tert-butoxycarbonyl)amino)-3-cyano-7-fluorobenzo[b]thiophen-4-yl)-6-chloro-2,8-difluoroquinazolin-4-yl)piperazine-1-carboxylate: To a mixture of tert-butyl 4-(7-bromo-6-chloro-2,8-difluoroquinazolin-4-yl)piperazine-1-carboxylate (155 g, 1.0 equiv) and tert-butyl (3-cyano-4-(5,5-dimethyl-1,3,2-dioxaborinan-2-yl)-7-fluorobenzo[b]thiophen-2-yl)carbamate (175 g, 1.3 equiv) in THF (3.10 L) were added cesium carbonate (326 g, 3.0 equiv) and Pd(DPEphos)Cl₂ (23.0 g, 0.1 equiv). The reaction was degassed and purged with nitrogen for 3 times. The reaction was stirred at 60 °C for 5 hours. The mixture was diluted with water (3.0 L) and extracted with ethyl acetate (1.0 L×3). The combined organic layers were washed with brine (2.0 L), dried over anhydrous sodium sulfate, concentrated and purified by column chromatography (SiO₂, Petroleum ether:ethyl acetate=20/1 to 10:1) to afford the title compound (65.0 g, 28% yield) as yellow solid; LCMS (ESI, M+1): m/z=675.1.

Step E tert-butyl 4-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate: To a solution of ((2R,7aS)-2-fluorotetrahydro-1H-pyrrolizin-7a(5H)-yl)methanol (17.3 g, 1.1 equiv) in tetrahydrofuran (500 mL) was added sodium hydride (12.4 g, 60% purity, 3.0 equiv) at 0 °C. The reaction was stirred at 0 °C for 0.5 hours. Then to the above mixture was added tert-butyl 4-(7-(2-((tert-butoxycarbonyl)amino)-3-cyano-7-fluorobenzo[b]thiophen-4-yl)-6-chloro-2,8-difluoroquinazolin-4-yl)piperazine-1-carboxylate (70.0 g, 1.0 equiv) in tetrahydrofuran (200 mL) at 0 °C. The reaction was stirred at 25 °C for 0.5 hours. The mixture was quenched with saturated ammonium chloride (700 mL) and extracted with ethyl acetate (200 mL×3). The combined organic layers were washed with brine (200 mL), dried over anhydrous sodium sulfate, concentrated, and triturated with acetonitrile (200 mL) at 20 °C for 30 mins to afford the title compound (76.0 g, 90% yield) as white solid; LCMS (ESI, M+1): m/z=814.3.

Step F. tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate: To a mixture of tert-butyl 4-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate (45.0 g, 1.0 equiv) in ethanol (900 mL) was added sodium hydroxide (360 mL, 2 M, 13.0 equiv). The reaction was stirred at 50 °C for 6 hours. The mixture was diluted with water (800 mL) and extracted with ethyl acetate (300 mL×3). The combined organic layers were washed with brine (300 mL), dried over anhydrous sodium sulfate, concentrated, and purified by reversed phase flash chromatography (0.1% formic acid) to afford the title compound (25.5 g, 71% yield) as yellow solid; ¹H NMR (400 MHz, METHANOL-d₄)_{8=8.01} (d, J=1.6 Hz, 1H), 7.32 (dd, J=5.2, 8.8 Hz, 1H), 7.24-7.14 (m, 1H), 5.48-5.23 (m, 1H), 4.38 (quin, J=11.6 Hz, 2H), 3.54-3.33 (m, 3H), 3.19-3.06 (m, 1H), 2.44-2.25 (m, 2H), 2.23-2.14 (m, 1H), 2.12-2.03 (m, 2H), 1.99-1.86 (m, 1H), 1.57 (s, 9H); LCMS (ESI, M+1): m/z=646.2.

Step G. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (680 mg, 1.0 equiv) and methyl piperidine-4-carboxylate (1.02 g, 6.8 equiv) in DMF (7 mL) were added PyBOP (1.37 g, 2.5 equiv) and DIEA (408 mg, 3.0 equiv). The reaction was stirred at 25 °C for 2 hours. The mixture was diluted with water (20 mL) and extracted with ethyl acetate (2×20 mL). The combined organic layers were washed with brine (15 mL), dried over anhydrous sodium sulfate, concentrated and purified by reversed phase flash chromatography [C18, 0.1% formic acid condition] to afford the title compound (580 mg, 71% yield) as yellow solid; LCMS (ESI, M+1): m/z=771.3.

Step H. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate (580 mg, 1.0 equiv) in DCM (10 mL) was added TFA (8 mL, 143 equiv). The mixture was stirred at 25 °C for 2 hours. The mixture was concentrated. The residue was basified with saturated NaHCO₃ solution (80 mL) and extracted with ethyl acetate (3×50 mL). The combined organic layers were dried over anhydrous sodium sulfate and concentrated to afford the title compound (500 mg, crude) as pink solid.

Step I. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate (450 mg, 1 equiv) in MeOH (10 mL) was added LiOH·H₂O (1.41 g, 50 equiv) in H₂O (6.5 mL). The reaction was stirred at 25 °C for 3 hours. The mixture was filtered and the filtrate was purified by reversed phase flash chromatography [C18, 0.1% formic acid condition] to afford the title compound (320 mg, 72% yield) as white solid; LCMS (ESI, M+1): m/z=657.2.

### Step J.

### 4-(4-(4-(1H-pyrazole-1-carbonyl)piperidin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (100 mg, 1.0 equiv) and 1H-pyrazole (51.8 mg, 5.0 equiv) in THF (1 mL) were added DIEA (118 mg, 6.0 equiv) and 2-Chloro-1-Methylpyridinium Iodide (155 mg, 4.0 equiv). The mixture was stirred at 25 °C for 1 hour. The mixture was concentrated and purified by prep-HPLC [Waters Xbridge Prep OBD C18 150×40 mm×10 μm; A: water (NH₄HCO₃), B: ACN, B %: 50%-80% over 20 min]. The desired fractions were diluted with water (50 mL) and extracted with dichloromethane (2×30 mL). The organic layers were dried over anhydrous sodium sulfate and concentrated to afford the title compound (6.71 mg, 5.7% yield) as yellow solid; ¹H NMR (400 MHz, CHLOROFORM-d) δ=8.29 (d, J=2.4 Hz, 1H), 7.76 (br d, J=9.6 Hz, 2H), 7.21 (br dd, J=4.8, 8.4 Hz, 1H), 7.09-6.98 (m, 1H), 6.53-6.46 (m, 1H), 5.64-5.45 (m, 2H), 5.39-5.19 (m, 1H), 4.52-4.37 (m, 2H), 4.31-4.14 (m, 2H), 4.07-3.94 (m, 1H), 3.47-3.10 (m, 5H), 3.05-2.91 (m, 1H), 2.30 (br s, 1H), 2.26-2.08 (m, 6H), 2.08-1.96 (m, 3H); LCMS (ESI, M+1): m/z=707.2.

### Example 130

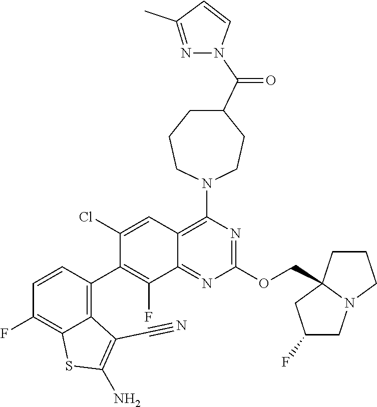 [SMILES: Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl) azepan-1-yl)quinazolin-7-yl)-7-fluorobenzo[b]thiophene-3-carbonitrile

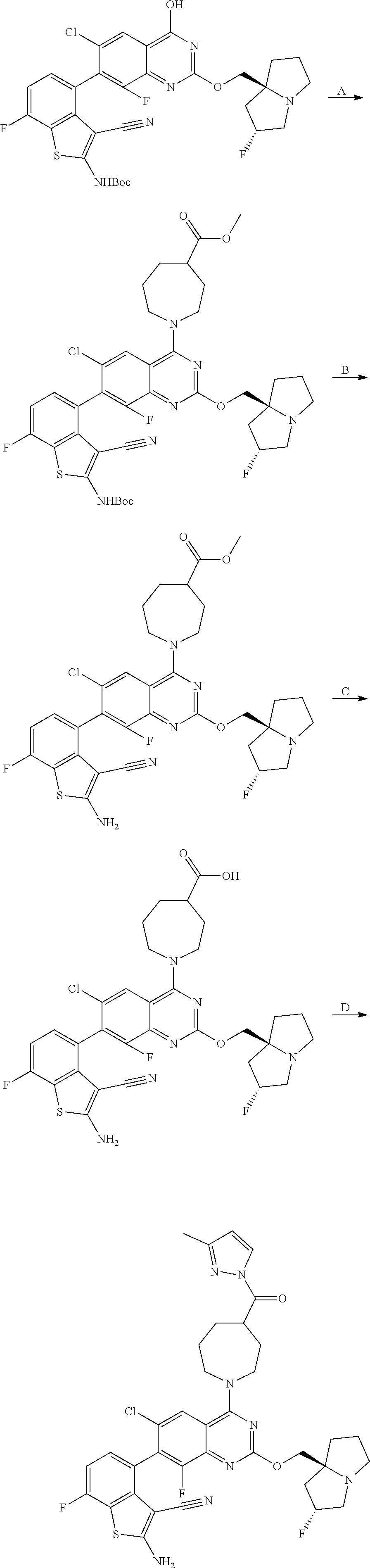

Step A. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of tert-(4-(6-chloro-8-fluoro-2-(((2R,7aS)-2-fluorotetrahydro-1H-pyrrolizin-7a(5H)-butyl yl)methoxy)-4-hydroxyquinazolin-7-yl)-3-cyano-7-fluorobenzo[b]thiophen-2-yl)carbamate (480 mg, 1.0 equiv) and methyl azepane-4-carboxylate (415 mg, 3.0 equiv) in DMSO (2 mL) were added PyBOP (853 mg, 2.0 equiv) and TEA (277 mg, 3.0 equiv). The reaction was stirred at 25 °C for 14 hours. The mixture was purified by reversed phase flash chromatography [water (0.1% NH₃·H₂O)/acetonitrile=1/4] to afford the title compound (320 mg, 54% yield) as yellow solid; LCMS (ESI, M+1): m/z=785.2.

Step B. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (310 mg, 1.0 equiv) in DCM (3 mL) was added TFA (4.5 mL) dropwise at 0 °C. The reaction was stirred at 20 °C for 1 hour. The mixture was concentrated. The residue was dissolved in ethyl acetate (15 mL) and saturated NaHCO₃ aqueous (15 mL) at 0 °C. The mixture was extracted with ethyl acetate (4×10 mL). The combined organic layers were washed brine (20 mL), dried over anhydrous sodium sulfate and concentrated to afford the title compound (320 mg, crude) as yellow solid; LCMS (ESI, M+1): m/z=685.3

Step C. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (320 mg, 1.0 equiv) in MeOH (3.5 mL) was added LiOH·H₂O (1.5 M, 934 μL, 3.0 equiv). The reaction was stirred at 20 °C for 2 hours. The pH of the mixture was adjusted to 8 with 1N HCl at 0 °C. The mixture was purified by reversed phase flash chromatography [water (0.1% formic acid)/acetonitrile=1/1] to afford the title compound (170 mg, 62% yield over two steps) as yellow solid; LCMS (ESI, M+1): m/z=671.2.

Step D. 2-amino-4-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl) azepan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (70.0 mg, 1.0 equiv) and 5-methyl-1H-pyrazole (21.6 mg, 2.5 equiv) in DMAc (1.0 mL) were added TEA (46.1 mg, 4.4 equiv) and EDCI (36.0 mg, 1.8 equiv) and HOBt (26.8 mg, 1.9 equiv). The mixture was stirred at 20 °C for 14 hours. The mixture was purified by reversed phase flash chromatography [water (0.1% formic acid)/acetonitrile=1/1, 1/4] and prep-HPLC [column: Waters Xbridge 150×25 mm×10 μm; mobile phase: (water (NH₄HCO₃)-acetonitrile); gradient: 62%-82% B over 8 minutes] and to afford the title compound (26.1 mg, 32% yield) as white solid; ¹H NMR (400 MHz, DMSO-d₆) δ=8.27 (d, J=2.8 Hz, 1H), 8.10 (s, 2H), 8.02-7.94 (m, 1H), 7.29-7.20 (m, 1H), 7.18-7.11 (m, 1H), 6.45 (d, J=2.8 Hz, 1H), 5.36-5.17 (m, 1H), 4.27-3.97 (m, 4H), 3.96-3.72 (m, 3H), 3.14-2.95 (m, 3H), 2.87-2.76 (m, 1H), 2.25 (d, J=2.0 Hz, 3H), 2.21-1.92 (m, 8H), 1.87-1.72 (m, 3H), 1.70-1.59 (m, 1H); LCMS (ESI, M+1): m/z=735.4.

### Example 131

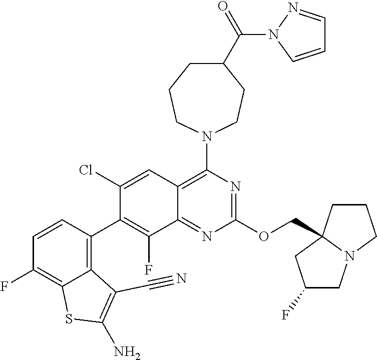 [SMILES: N#Cc1c(N)sc2c(F)ccc(-c3c(Cl)cc4c(N5CCCC(C(=O)n6cccn6)CC5)nc(OC[C@@]56CCCN5C[C@H](F)C6)nc4c3F)c12]

### 4-(4-(4-(1H-pyrazole-1-carbonyl) azepan-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

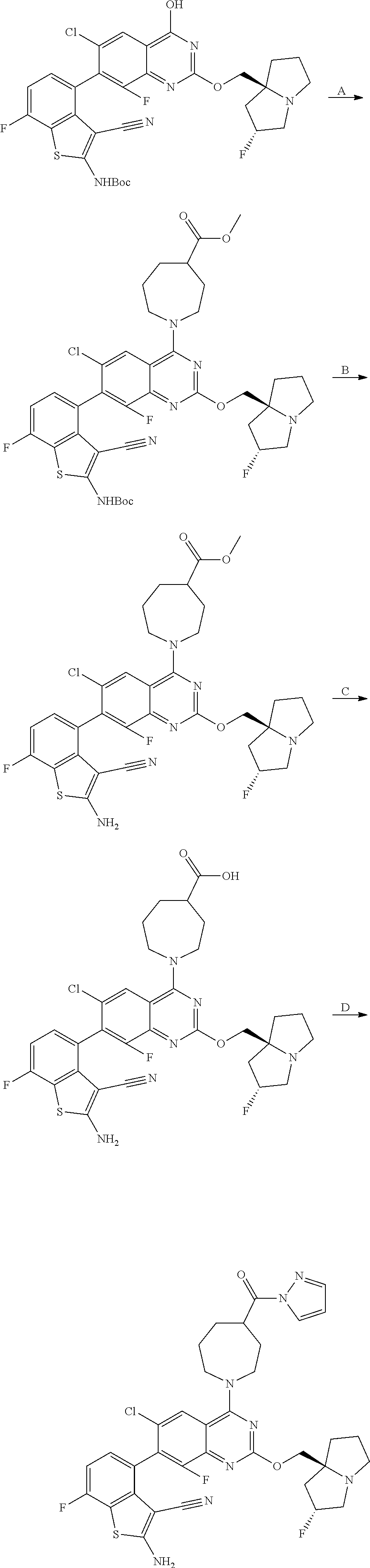

Step A. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (480 mg, 1.0 equiv) and methyl azepane-4-carboxylate (415 mg, 3.0 equiv) in DMSO (2 mL) were added PyBOP (853 mg, 2.0 equiv) and TEA (277 mg, 3.0 equiv). The reaction was stirred at 25 °C for 14 hours. The mixture was purified by reversed phase flash chromatography [water (0.1% NH₃·H₂O)/acetonitrile=1/4] to afford the title compound (320 mg, 54% yield) as yellow solid; LCMS (ESI, M+1): m/z=785.2.

Step B. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (310 mg, 1.0 equiv) in DCM (3 mL) was added TFA (4.5 mL) dropwise at 0 °C. The reaction was stirred at 20 °C for 1 hour. The mixture was concentrated. The residue was dissolved in ethyl acetate (15 mL) and saturated NaHCO₃ aqueous (15 mL) at 0 °C. The mixture was extracted with ethyl acetate (4×10 mL). The combined organic layers were washed brine (20 mL), dried over anhydrous sodium sulfate and concentrated to afford the title compound (320 mg, crude) as yellow solid; LCMS (ESI, M+1): m/z=685.3

Step C. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (320 mg, 1.0 equiv) in MeOH (3.5 mL) was added LiOH·H₂O (1.5 M, 934 μL, 3.0 equiv). The reaction was stirred at 20 °C for 2 hours. The pH of the mixture was adjusted to 8 with 1N HCl at 0 °C. The mixture was purified by reversed phase flash chromatography [water (0.1% formic acid)/acetonitrile=1/1] to afford the title compound (170 mg, 62% yield over two steps) as yellow solid; LCMS (ESI, M+1): m/z=671.2.

Step D. 4-(4-(4-(1H-pyrazole-1-carbonyl) azepan-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (60.0 mg, 1.0 equiv) and 1H-pyrazole (15.3 mg, 2.5 equiv) in DMAc (0.6 mL) were added TEA (39.5 mg, 4.4 equiv), EDCI (30.8 mg 1.8 equiv) and HOBt (22.9 mg, 1.9 equiv). The reaction was stirred at 20 °C for 14 hours. The mixture was purified by prep-HPLC [column: Waters Xbridge 150×25 mm×5 μm; mobile phase: water (NH₄HCO₃)-acetonitrile; gradient: 50%-65% B over 53 minutes] to afford the title compound (33 mg, 50% yield) as white solid; ¹H NMR (400 MHz, DMSO-d6) δ=8.40 (d, J=2.8 Hz, 1H), 8.10 (s, 2H), 8.00 (s, 1H), 7.90 (s, 1H), 7.25 (dt, J=5.2, 9.2 Hz, 1H), 7.19-7.11 (m, 1H), 6.62 (dd, J=1.6, 2.8 Hz, 1H), 5.36-5.17 (m, 1H), 4.27-4.14 (m, 2H), 4.13-4.07 (m, 1H), 4.06-3.99 (m, 1H), 3.99-3.74 (m, 3H), 3.14-3.02 (m, 2H), 3.00 (s, 1H), 2.85-2.76 (m, 1H), 2.38-2.26 (m, 1H), 2.21-1.94 (m, 7H), 1.88-1.61 (m, 4H); LCMS (ESI, M+1): m/z=721.1.

### Example 132

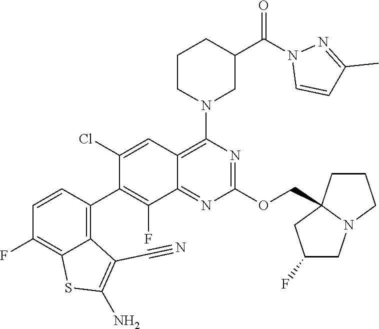 [SMILES: Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)C2)n1]

### 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl)piperidin-1-yl)quinazolin-7-yl)-7-fluorobenzo[b]thiophene-3-carbonitrile

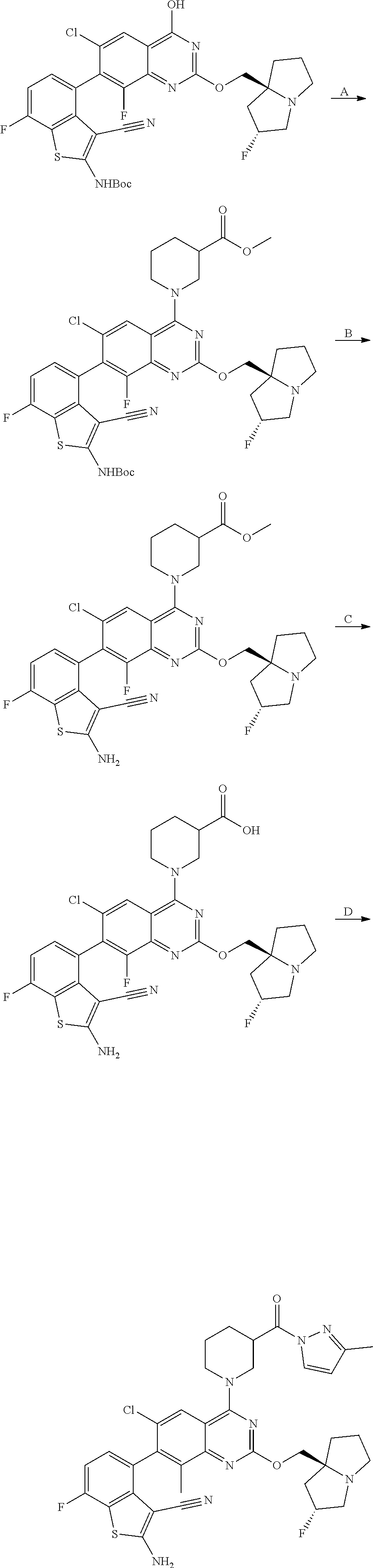

Step A. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-3-carboxylate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv), TEA (235 mg, 3.0 equiv) and benzotriazol-1-yloxyl-tris-(pyrrolidino)-phosphonium hexafluorophosphate (604 mg, 1.5 equiv) in DMSO (5 mL) was added methyl piperidine-3-carboxylate (222 mg, 2.0 equiv). The reaction was stirred at 30 °C for 12 hours. The mixture was diluted with water (10 mL) and extracted with ethyl acetate (3×10 mL). The combined organic layers were washed with brine (20 mL), dried over anhydrous sodium sulfate, concentrated and purified by reversed phase flash chromatography [C18, 0.1% formic acid condition] to afford the title compound (330 mg, 52% yield) as yellow solid; LCMS (ESI, M+1): m/z=771.3.

Step B. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-3-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-3-carboxylate (330 mg, 1.0 equiv) in DCM (1.5 mL) was added TFA (1.5 mL). The reaction was stirred at 0 °C for 0.5 hours. The mixture was adjusted to pH>8 with saturated NaHCO₃ aqueous (10 mL) at 0 °C and extracted with DCM (3×5 mL). The combined organic layers were washed with brine (10 mL), dried over anhydrous sodium sulfate and concentrated to afford the title compound (300 mg, crude) as yellow solid; LCMS (ESI, M+1): m/z=671.3.

Step C. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-3-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-3-carboxylate (300 mg, 1.0 equiv) in THF (2 mL) and MeOH (0.5 mL) was added LiOH·H₂O (56.3 mg, 3.0 equiv) in H₂O (0.5 mL). The reaction was stirred at 20 °C for 1 hour. The mixture was concentrated and purified by reversed phase flash chromatography [C18, 0.1% formic acid condition] to afford the title compound (200 mg, 66% yield) as yellow solid; LCMS (ESI, M+1): m/z=657.2.

Step D. 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl)piperidin-1-yl)quinazolin-7-yl)-7-fluorobenzo[b]thiophene-3-carbonitrile: To a mixture of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-3-carboxylic acid (100 mg, 1.0 equiv), DIEA (59.0 mg, 3.0 equiv), EDCI (43.8 mg, 1.5 equiv) and HOBt (30.9 mg, 1.5 equiv) in DMF (1 mL) was added 3-Methylpyrazole (25.0 mg, 2.0 equiv). The reaction was stirred at 20 °C for 1 hour. The reaction was filtered and purified by reversed phase flash chromatography [C18, neutral condition] and prep-HPLC [column: Waters Xbridge 150×25 mm×5 μm; A: water(ammonia hydroxide v/v); B: ACN, B %: 64%-94% B over 9 min] to afford the title compound (12.0 mg, 10% yield) as white solid; ¹H NMR (400 MHz, DMSO-d₆) δ=8.31 (br s, 1H), 8.11 (s, 2H), 7.89 (br d, J=4.8 Hz, 1H), 7.29-7.20 (m, 1H), 7.19-7.10 (m, 1H), 6.47 (t, J=2.8 Hz, 1H), 5.36-5.14 (m, 1H), 4.51-4.36 (m, 1H), 4.10-4.08 (m, 2H), 4.05-3.89 (m, 2H), 3.61-3.46 (m, 2H), 3.09-2.96 (m, 3H), 2.85-2.76 (m, 1H), 2.26 (d, J=14.8 Hz, 3H), 2.20-2.10 (m, 2H), 2.05-1.97 (m, 2H), 1.93-1.73 (m, 6H); LCMS (ESI, M+1): m/z=721.3.

### Example 133

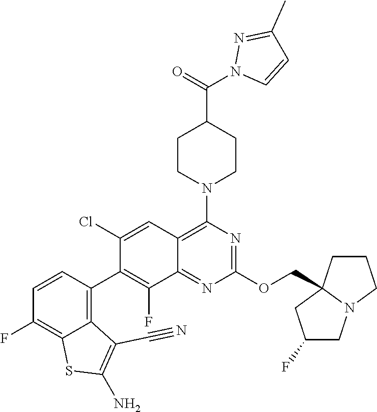 [SMILES: Cc1ccn(C(=O)C2CCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl)piperidin-1-yl)quinazolin-7-yl)-7-fluorobenzo[b]thiophene-3-carbonitrile

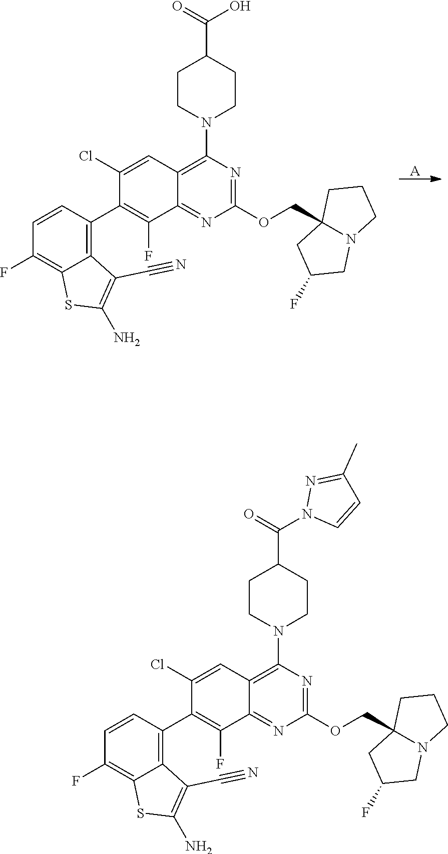

Step A. 2-amino-4-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl)piperidin-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (140 mg, 1.0 equiv) and 3-methyl-1H-pyrazole (87.5 mg, 5.0 equiv) in THF (1.5 mL) was added DIEA (165 mg, 6.0 equiv) and 2-Chloro-1-Methylpyridinium Iodide (218 mg, 4.0 equiv). The mixture was stirred at 25 °C for 1 hour. The mixture was concentrated and purified by prep-HPLC [Waters Xbridge Prep OBD C18 150×40 mm×10 μm; A: water (NH₄HCO₃), B: ACN, B %: 52%-82% over 20 min]. The desired fractions were diluted with water (10 mL) and extracted with dichloromethane (2×10 mL). The organic layers were dried over anhydrous sodium sulfate and concentrated to afford the title compound (24.4 mg, 15.0% yield) as yellow solid; ¹H NMR (400 MHz, CHLOROFORM-d) δ=8.17 (d, J=2.6 Hz, 1H), 7.74 (d, J=1.4 Hz, 1H), 7.20 (dd, J=5.0, 8.4 Hz, 1H), 7.01 (t, J=8.8 Hz, 1H), 6.28 (d, J=2.8 Hz, 1H), 5.74 (br s, 2H), 5.37-5.17 (m, 1H), 4.53-4.39 (m, 2H), 4.30-4.14 (m, 2H), 4.04-3.89 (m, 1H), 3.47-3.12 (m, 5H), 2.97 (dt, J=5.2, 9.1 Hz, 1H), 2.36 (s, 3H), 2.28 (br s, 1H), 2.24-2.07 (m, 6H), 2.00-1.91 (m, 3H); LCMS (ESI, M+1): m/z=721.4.

### Example 134

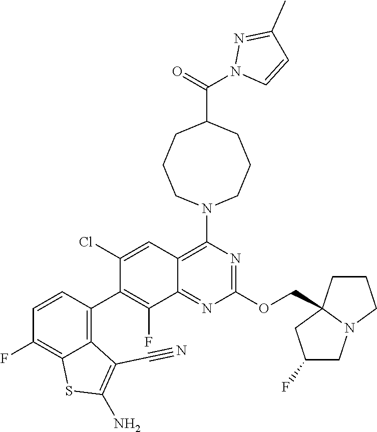 [SMILES: Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CCC2)n1]

### 2-amino-4-(6-chloro-8-fluoro-2-(((2R,7aS)-2-fluorotetrahydro-1H-pyrrolizin-7a(5H)-yl)methoxy)-4-(5-(3-methyl-1H-pyrazole-1-carbonyl) azocan-1-yl)quinazolin-7-yl)-7-fluorobenzo[b]thiophene-3-carbonitrile

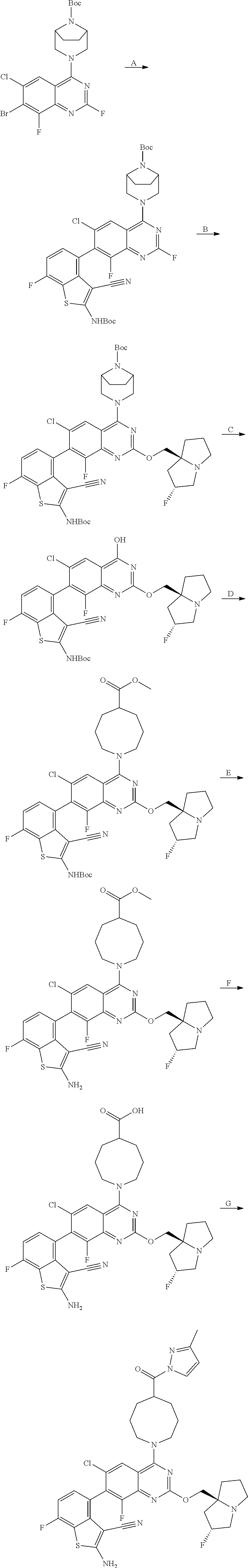

Step A. tert-butyl (1R,5S)-3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate: To a mixture of tert-butyl (1R,5S)-3-(7-bromo-6-chloro-2,8-difluoroquinazolin-4-yl)-3,8-diazabicyclo[3.2.1]octane-8-carboxylate (10 g, 1.0 equiv) and tert-butyl (3-cyano-4-(5,5-dimethyl-1,3,2-dioxaborinan-2-yl)-7-fluorobenzo[b]thiophen-2-yl)carbamate (13.2 g, 1.6 equiv) in THF (150 mL) were added Cs₂CO₃ (13.3 g, 2.0 equiv) and DPEPhosPdCl₂ (1.40 g, 0.10 equiv). The reaction was degassed and purged with nitrogen 3 times. The reaction was stirred at 90 °C for 3 hours. The mixture was diluted with H₂O (90 mL) and extracted with ethyl acetate (3×60 mL). The combined organic layers were washed with brine, dried over anhydrous sodium sulfate, concentrated. The residue was purified by column chromatography (SiO₂, petroleum ether/ethyl acetate=50/1 to 3/1) followed by reversed phase flash [water (0.1% FA)/acetonitrile] to afford the title compound (3.1 g, 22% yield) as yellow solid; LCMS (ESI, M+1): m/z=701.2.

Step B. tert-butyl (1R,5S)-3-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate: To a solution of ((2R,7aS)-2-fluorotetrahydro-1H-pyrrolizin-7a(5H)-yl)methanol (724 mg, 1.1 equiv) in THF (25 mL) was added NaH (496 mg, 60% purity, 3.0 equiv) at 0 °C. The reaction was stirred at 0 °C for 0.5 hours and then tert-butyl (1R,5S)-3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (2.9 g, 1.0 equiv) in THF (25 mL) was added dropwise at 0 °C. The reaction was stirred at 25 °C for 0.5 hours. The mixture was quenched with water (50 mL) and extracted with ethyl acetate (3×30 mL). The combined organic layers were dried over anhydrous sodium sulfate and concentrated to afford the title compound (3.05 g, crude) as yellow solid.

Step C. tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate: To a solution of tert-butyl (1R,5S)-3-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (3.05 g, 1.0 equiv) in EtOH (90 mL) and H₂O (9 mL) was added NaOH (735 mg, 5.1 equiv). The reaction was stirred at 50 °C for 16 hours. The mixture was concentrated in vacuum, diluted with water (20 mL) and adjusted to pH=6 with saturated aqueous citric acid. The aqueous phase was extracted with ethyl acetate (3×20 mL). The combined organic layers were dried over anhydrous sodium sulfate, concentrated and purified by reversed phase flash chromatography [water (0.1% FA)/acetonitrile] to afford the title compound (1.06 g, 42% yield 0.55FA) as yellow solid; LCMS (ESI, M+1): m/z=646.1.

Step D. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-5-carboxylate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamateb (300 mg, 1.0 equiv, 0.55FA) and methyl azocane-5-carboxylate (145 mg, 1.6 equiv, HCl) in DMSO (3 mL) were added PyBOP (363 mg, 1.7 equiv) and TEA (376 mg, 8.3 equiv). The mixture was stirred at 30 °C for 6 hours. Then PyBOP (242 mg, 1.04 equiv) was added. The reaction was stirred at 30 °C for another 2 hours. The mixture was filtered and purified by reversed phase flash [water (0.1% FA)/acetonitrile] to afford the title compound (185 mg, 47% yield) as yellow solid; LCMS (ESI, M+1): m/z=799.2.

Step E. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-5-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-5-carboxylate (185 mg, 1.0 equiv) in DCM (1 mL) was added TFA (1.54 g, 58 equiv). The reaction was stirred at 25 °C for 0.5 hours. The mixture was concentrated and basified with saturated aqueous NaHCO₃ (3 mL). The aqueous phase was extracted with ethyl acetate (3×4 mL). The combined organic layers were dried over anhydrous sodium sulfate and concentrated to afford the title compound (180 mg, crude) as yellow solid.

Step F. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-5-carboxylic acid:

To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-5-carboxylate (180 mg, 1.0 equiv) in EtOH (3 mL) was added NaOH (1.0 M, 1.5 mL, 5.8 equiv). The reaction was stirred at 25 °C for 2 hours. The mixture was concentrated and purified by reversed phase flash [water (0.1% FA)/acetonitrile] to afford the title compound (120 mg, 68% yield) as white solid; LCMS (ESI, M+1): m/z=685.2.

Step G. 2-amino-4-(6-chloro-8-fluoro-2-(((2R,7aS)-2-fluorotetrahydro-1H-pyrrolizin-7a(5H)-yl)methoxy)-4-(5-(3-methyl-1H-pyrazole-1-carbonyl) azocan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-5-carboxylic acid (60 mg, 1.0 equiv) and 3-methyl-1H-pyrazole (35.9 mg, 5.0 equiv) in THF (1.5 mL) were added DIEA (67.9 mg, 6.0 equiv) and 2-Chloro-1-Methylpyridinium Iodide (89.5 mg, 4.0 equiv). The reaction was stirred at 25 °C for 0.5 hours. The mixture was filtered and purified by prep-HPLC [column: Waters Xbridge 150×25 mm×5 μm; A: water (NH₄HCO₃), B: ACN, B %: 60%-90% over 9 min]. The aqueous was extracted with ethyl acetate (2×10 mL). The combined organic layers were dried over anhydrous sodium sulfate and concentrated to afford the title compound (7.74 mg, 12% yield) as yellow solid; ¹H NMR (400 MHz, CHLOROFORM-d) δ=8.13 (d, J=2.8 Hz, 1H), 7.91 (s, 1H), 7.20 (dd, J=5.2, 8.0 Hz, 1H), 7.02 (t, J=8.4 Hz, 1H), 6.28-6.20 (m, 1H), 5.78-5.61 (m, 2H), 5.38-5.18 (m, 1H), 4.31-4.14 (m, 4H), 4.05-3.95 (m, 1H), 3.93-3.74 (m, 2H), 3.34-3.15 (m, 3H), 3.04-2.91 (m, 1H), 2.28 (s, 3H), 2.26-2.10 (m, 7H), 2.10-2.05 (m, 3H), 1.98-1.87 (m, 4H); LCMS (ESI, M+1): m/z=749.3.

### Example 135

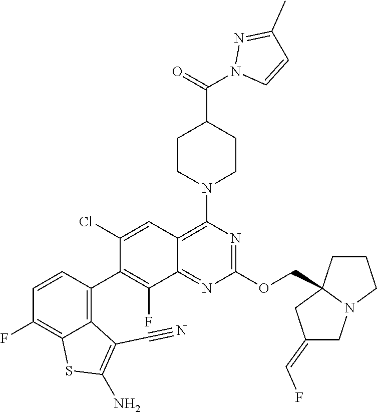 [SMILES: Cc1ccn(C(=O)C2CCN(c3nc(OC[C@@]45CCCN4C/C(=C\F)C5)nc4c(F)c(-c5ccc(F)c6sc(N)c(C#N)c56)c(Cl)cc34)CC2)n1]

### 2-amino-4-(6-chloro-8-fluoro-2-(((S,Z)-2-(fluoromethylene)tetrahydro-1H-pyrrolizin-7a(5H)-yl)methoxy)-4-(4-(3-methyl-1H-pyrazole-1-carbonyl)piperidin-1-yl)quinazolin-7-yl)-7-fluorobenzo[b]thiophene-3-carbonitrile

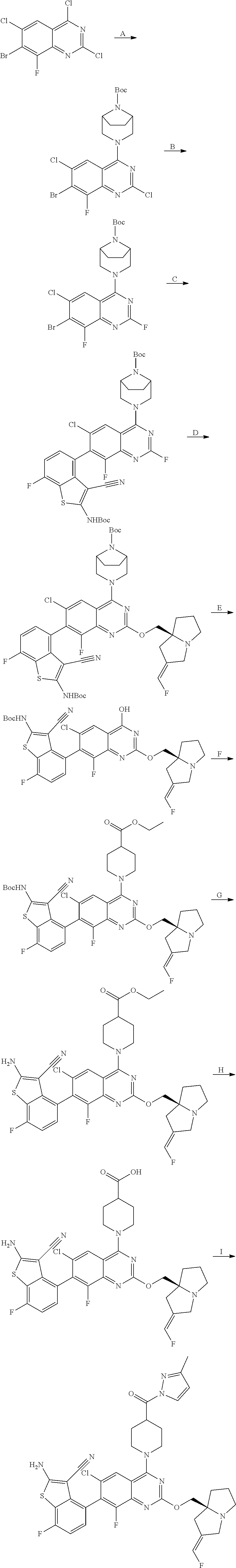

Step A. tert-butyl (1R,5S)-3-(7-bromo-2,6-dichloro-8-fluoroquinazolin-4-yl)-3,8-diazabicyclo[3.2.1]octane-8-carboxylate: To a solution of 7-bromo-2,4,6-trichloro-8-fluoroquinazoline (168 g, 1.0 equiv) and TEA (94.7 g, 2.0 equiv) in DCM (1680 mL) was added tert-butyl (1R,5S)-3,8-diazabicyclo[3.2.1]octane-8-carboxylate (94.4 g, 0.95 equiv) slowly at −40 °C. The mixture was stirred at −40 °C for 0.5 hours. The mixture was quenched with water (1.2 L) and extracted with dichloromethane (1.0 L). The combined organic layers were dried over anhydrous sodium sulfate, concentrated under vacuum to afford the title compound (275 g, crude) as yellow solid; ¹H NMR (400 MHz, CHLOROFORM-d) δ=7.76 (d, J=2.0 Hz, 1H), 4.53-4.26 (m, 4H), 3.79-3.52 (m, 2H), 2.04-1.88 (m, 2H), 1.72 (br d, J=6.4 Hz, 2H), 1.53 (s, 9H); LCMS (ESI, M+1): m/z=507.0.

Step B. tert-butyl (1R,5S)-3-(7-bromo-6-chloro-2,8-difluoroquinazolin-4-yl)-3,8-diazabicyclo[3.2.1]octane-8-carboxylate: To a mixture of tert-butyl (1R,5S)-3-(7-bromo-2,6-dichloro-8-fluoroquinazolin-4-yl)-3,8-diazabicyclo[3.2.1]octane-8-carboxylate (200 g, 1.0 equiv) and KF (230 g, 10 equiv) in DMSO (1000 mL) was added 1,4,7,10,13,16-hexaoxacyclooctadecane (10.4 g, 0.1 equiv). The reaction was stirred at 120 °C for 4 hours. The mixture was diluted with water (1.5 L) and extracted with ethyl acetate (2×1000 mL). The combined organic layers were dried over anhydrous sodium sulfate and concentrated under vacuum to give a residue. The crude product was triturated with (Petroleum ether: ethyl acetate=10/1) (100 mL) at 20 °C for 30 minutes to afford the title compound (172 g, 86% yield) as yellow solid; ¹H NMR (400 MHz, DMSO-d6) δ=8.14 (d, J=2.0 Hz, 1H), 8.16-8.06 (m, 1H), 4.39 (br d, J=12.0 Hz, 2H), 4.29-4.17 (m, 2H), 3.66 (br d, J=12.8 Hz, 2H), 1.82-1.72 (m, 2H), 1.61 (br d, J=7.6 Hz, 2H), 1.46 (s, 9H).

Step C. tert-butyl (1R,5S)-3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate: To a mixture of tert-butyl (1R,5S)-3-(7-bromo-6-chloro-2,8-difluoroquinazolin-4-yl)-3,8-diazabicyclo[3.2.1]octane-8-carboxylate (1 g, 1.0 equiv) and tert-butyl (3-cyano-4-(5,5-dimethyl-1,3,2-dioxaborinan-2-yl)-7-fluorobenzo[b]thiophen-2-yl)carbamate (1.65 g, 2.0 equiv) in THF (30 mL) were added Cs₂CO₃ (2.0 g, 3.0 equiv) and Pd(DPEphos)Cl₂ (140 mg, 0.1 equiv). The reaction was degassed and purged with N₂ for 3 times. The reaction was stirred at 60 °C for 3 hours. The mixture was diluted with water (100 mL) and extracted with ethyl acetate (4×50 mL). The combined organic layers were washed with brine (30 mL) dried over anhydrous sodium sulfate, concentrated and purified by reversed phase flash [0.1% FA condition] to afford the title compound (1.6 g, 89% yield) as yellow oil; LCMS (ESI, M+1): m/z=701.2.

Step D. tert-butyl (1R,5S)-3-(7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-3,8-diazabicyclo[3.2.1]octane-8-carboxylate: To a solution of (S,Z)-(2-(fluoromethylene)tetrahydro-1H-pyrrolizin-7a(5H)-yl)methanol (365 mg, 1.1 equiv) in THF (20 mL) was added NaH (116 mg, 60% purity, 1.5 equiv) at 0 °C. The reaction was stirred at 0 °C for 15 minutes. Tert-butyl (1R,5S)-3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (1.6 g, 1.0 equiv) was added into the mixture. The reaction was stirred at 20 °C for 10 minutes. The mixture was quenched with water (40 mL) at 0 °C and extracted with ethyl acetate (4×20 mL). The combined organic layers were washed with brine (40 mL), dried over anhydrous sodium sulfate and concentrated to afford the title compound (1.7 g, crude) as yellow solid; LCMS (ESI, M+1): m/z=852.3.

Step E. tert-butyl (4-(6-chloro-8-fluoro-2-(((S,Z)-2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate: To a solution of tert-butyl (1R,5S)-3-(7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-3,8-diazabicyclo[3.2.1]octane-8-carboxylate (1.7 g, 1.0 equiv) in EtOH (15 mL) was added NaOH (239 mg, 3.0 equiv) in H₂O (1.5 mL). The reaction was stirred at 60 °C for 12 hours. The pH of the mixture was adjusted to 8 with 1N HCl at 0 °C. The mixture was purified by reversed phase flash [0.1% FA condition] to afford the title compound (255 mg, 19% yield) as yellow solid; LCMS (ESI, M+1): m/z=658.3.

Step F ethyl 1-(7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)piperidine-4-carboxylate: To a solution of tert-butyl (4-(6-chloro-8-fluoro-2-(((S,Z)-2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate (140 mg, 1.0 equiv) and methyl piperidine-4-carboxylate (167 mg, 5.0 equiv) in DMSO (1 mL) were added PyBOP (166 mg, 1.5 equiv) and TEA (172 mg, 8.0 equiv). The reaction was stirred at 35 °C for 12 hours. The mixture was diluted with water (10 mL) and extracted with ethyl acetate (4×5 mL). The combined organic layers were washed with brine (5 mL), dried over anhydrous sodium sulfate and concentrated to afford the title compound (200 mg, crude) as brown oil; LCMS (ESI, M+1): m/z=797.2.

Step G. ethyl 1-(7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)piperidine-4-carboxylate: To a solution of ethyl 1-(7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)piperidine-4-carboxylate (190 mg, 1.0 equiv) in DCM (1 mL) was added TFA (1.5 mL) at 0 °C. The reaction was stirred at 20 °C for 0.5 hours. The mixture was concentrated. The residue was dissolved in ethyl acetate (10 mL) and saturated NaHCO₃ aqueous (10 mL) at 0 °C. The mixture was extracted with ethyl acetate (4×10 mL). The combined organic layers were washed brine (20 mL), dried over anhydrous sodium sulfate, and concentrated to afford the title compound (180 mg, crude) as brown solid; LCMS (ESI, M+1): m/z=697.2.

Step H. 1-(7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)piperidine-4-carboxylic acid: To a solution of ethyl 1-(7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)piperidine-4-carboxylate (170 mg, 1.0 equiv) in MeOH (1 mL) was added LiOH (1.5 M in H₂O, 488 μL, 3.0 equiv). The reaction was stirred at 20 °C for 2 hours. The pH of the mixture was adjusted to 8 with 1N HCl at 0 °C. The mixture was purified by reversed phase flash [0.1% FA condition] to afford the title compound (100 mg, 55% yield) as yellow solid; LCMS (ESI, M+1): m/z=669.2.

Step I. 2-amino-4-(6-chloro-8-fluoro-2-(((S,Z)-2-(fluoromethylene)tetrahydro-1H-pyrrolizin-7a(5H)-yl)methoxy)-4-(4-(3-methyl-1H-pyrazole-1-carbonyl)piperidin-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)piperidine-4-carboxylic acid (100 mg, 1.0 equiv) and 3-methyl-1H-pyrazole (74 mg, 6.0 equiv) in DMAc (1 mL) were added HOBt (30 mg, 1.5 equiv), EDCI (43 mg, 1.5 equiv) and TEA (121 mg, 8.0 equiv). The reaction was stirred at 40 °C for 2 hours. The mixture was purified by prep-HPLC [column: Waters Xbridge 150×25 mm×5 μm; mobile phase: water (NH₄HCO₃)-ACN; gradient: 58%-78% B over 10 minutes] to afford the title compound (2.29 mg, 2% yield) as off-white solid; ¹H NMR (400 MHz, CD₃OD) δ=8.22 (d, J=2.8 Hz, 1H), 7.89 (s, 1H), 7.22 (dd, J=5.2, 8.4 Hz, 1H), 7.09-7.00 (m, 1H), 6.82-6.55 (m, 1H), 6.40 (d, J=2.8 Hz, 1H), 4.59-4.50 (m, 2H), 4.45-4.30 (m, 2H), 4.10-3.91 (m, 2H), 3.64-3.48 (m, 3H), 2.88-2.74 (m, 2H), 2.56-2.48 (m, 1H), 2.34 (s, 3H), 2.24-2.14 (m, 3H), 2.11-1.87 (m, 6H); LCMS (ESI, M+1): m/z=733.3.

### Example 136

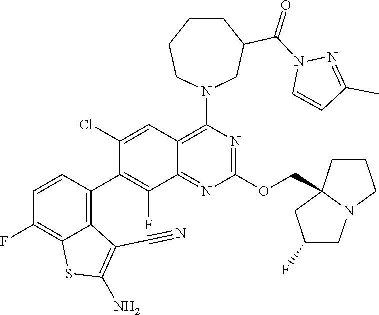 [SMILES: Cc1ccn(C(=O)C2CCCCN(c3nc(OC[C@@]45CCCN4C[C@H](F)C5)nc4c(F)c(-c5ccc(F)c6sc(N)c(C#N)c56)c(Cl)cc34)C2)n1]

### 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl) azepan-1-yl)quinazolin-7-yl)-7-fluorobenzo[b]thiophene-3-carbonitrile

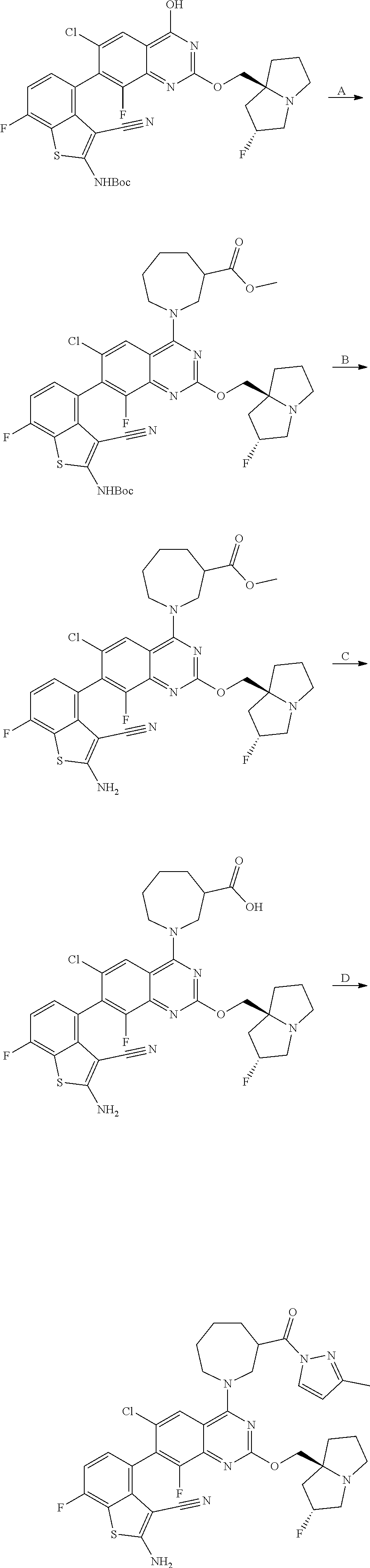

Step A. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-3-carboxylate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (270 mg, 1.0 equiv) and methyl azepane-3-carboxylate hydrogen chloride (131 mg, 1.6 equiv) in DMSO (3 mL) were added PyBOP (326 mg, 1.5 equiv) and TEA (127 mg, 3.0 equiv). The reaction was stirred at 35 °C for 48 hours. The mixture was purified by reversed phase flash chromatography [water (0.1% formic acid)/acetonitrile=1/1] to afford the title compound (192 mg, 56% yield) as brown oil; LCMS (ESI, M+1): m/z=785.2.

Step B. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-3-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-3-carboxylate (185 mg, 1.0 equiv) in DCM (1.5 mL) was added TFA (2.0 mL) at 0 °C. The mixture was stirred at 20 °C for 1 hour. The mixture was concentrated. The residue was dissolved in ethyl acetate (10 mL) and saturated NaHCO₃ aqueous (10 mL) at 0 °C. The mixture was extracted with ethyl acetate (4×10 mL). The combined organic layers were washed with brine (20 mL), dried over anhydrous sodium sulfate and concentrated to afford the title compound (160 mg, 82% yield) as yellow solid; LCMS (ESI, M+1): m/z=685.3.

Step C. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-3-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-3-carboxylate (150 mg, 1.0 equiv) in MeOH (1 mL) was added LiOH H₂O (1.5 M, 876 μL, 6.0 equiv). The reaction was stirred at 20 °C for 1 hour. The pH of the mixture was adjusted to 8 with 1N HCl at 0 °C. The mixture was purified by reversed phase flash chromatography [water (0.1% formic acid)/acetonitrile=1/1] to afford the title compound (74 mg, 50% yield) as yellow solid; LCMS (ESI, M+1): m/z=671.2.

Step D. 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl) azepan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-3-carboxylic acid (60.0 mg, 1.0 equiv) and 3-methyl-1H-pyrazole (18.3 mg, 2.5 equiv) in DMAc (0.6 mL) were added TEA (40.7 mg, 4.5 equiv), EDCI (34.3 mg, 2.0 equiv) and HOBt (24.2 mg, 2.0 equiv). The reaction was stirred at 20 °C for 14 hours. The mixture was purified by prep-HPLC [column: Waters Xbridge 150×25 mm×5 μm; mobile phase: water (NH₄HCO₃)-acetonitrile; gradient: 52%-72% B over 10 minutes] to afford the title compound (3.41 mg, 5% yield) as white solid; ¹H NMR (400 MHz, DMSO-d6) δ=8.33 (t, J=2.4 Hz, 1H), 8.11 (s, 2H), 8.06-7.96 (m, 1H), 7.31-7.20 (m, 1H), 7.19-7.10 (m, 1H), 6.49 (t, J=2.4 Hz, 1H), 5.30-5.06 (m, 1H), 4.49-4.36 (m, 1H), 4.23-4.08 (m, 2H), 4.07-3.88 (m, 3H), 3.07-2.87 (m, 3H), 2.83-2.72 (m, 1H), 2.57 (s, 1H), 2.26 (s, 3H), 2.11-1.88 (m, 7H), 1.85-1.74 (m, 2H), 1.72-1.62 (m, 2H), 1.56-1.40 (m, 1H); LCMS (ESI, M+1): m/z=735.3.

### Example 137

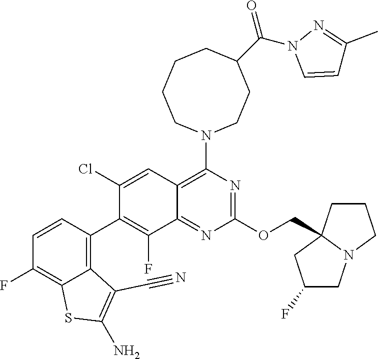 [SMILES: Cc1ccn(C(=O)C2CCCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl) azocan-1-yl)quinazolin-7-yl)-7-fluorobenzo[b]thiophene-3-carbonitrile

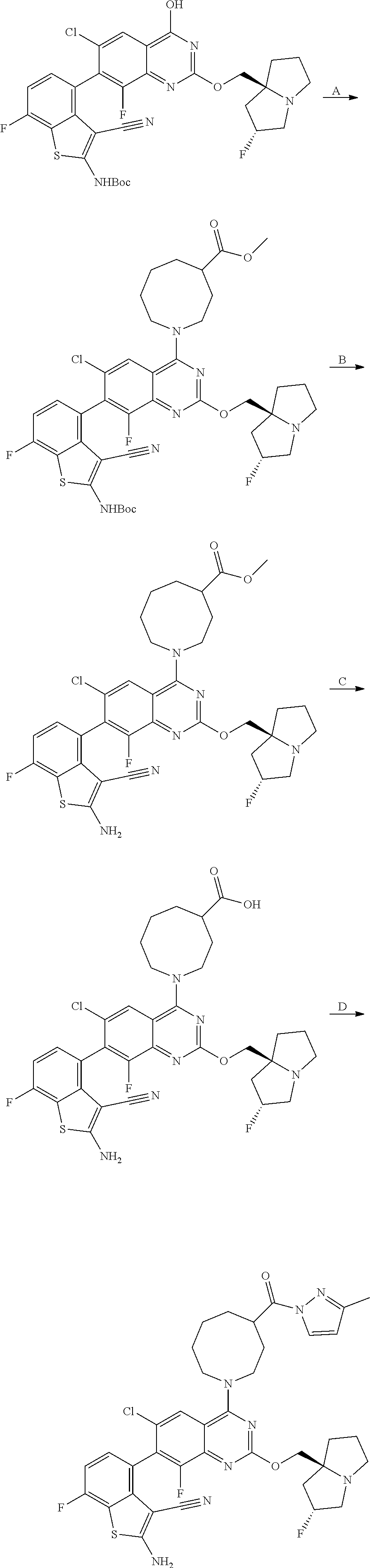

Step A. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-4-carboxylate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (300 mg, 1 equiv) and TEA (235 mg, 5 equiv) in DMSO (3 mL) was added PyBOP (362 mg, 1.5 equiv). After stirring at 25 °C for 20 minutes, methyl azocane-4-carboxylate (145 mg, 1.5 equiv, HCl salt) was added to the resulting mixture. The reaction was stirred at 25 °C for 2 hours. The mixture was filtered and the filtrate was purified by reversed phase flash chromatography [C18, 0.1% formic acid condition] to afford the title compound (180 mg, 45% yield) as yellow solid; ¹H NMR (400 MHz, dimethyl sulfoxide-d₆) δ 8.01 (s, 1H), 7.41-7.15 (m, 2H), 5.59-5.32 (m, 1H), 4.42-4.21 (m, 2H), 4.20-4.09 (m, 2H), 4.02-3.88 (m, 2H), 3.65-3.61 (m, 3H), 3.32-3.24 (m, 2H), 3.11-3.00 (m, 1H), 2.79-2.65 (m, 2H), 2.35-2.16 (m, 4H), 2.02-1.85 (m, 6H), 1.77-1.59 (m, 4H), 1.52-1.49 (m, 9H); LCMS (ESI, M+1): m/z=799.3.

Step B. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-4-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-4-carboxylate (90 mg, 1 equiv) in DCM (167 μL) was added TFA (257 mg, 20 equiv). The reaction was stirred at 25 °C for 1 hour. The mixture was concentrated. The residue was diluted with ethyl acetate (3 mL) and neutralized with saturated aqueous NaHCO₃ solution (3 mL). The mixture was extracted with ethyl acetate (2×3 mL). The combined organic layers were dried over anhydrous sodium sulfate and concentrated to afford the title compound (75 mg, 86% yield) as yellow solid; LCMS (ESI, M+1): m/z=699.2.

Step C. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-4-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-4-carboxylate (75 mg, 1.0 equiv) in EtOH (1.2 mL) was added a solution of NaOH (42.9 mg, 10 equiv) in H₂O (0.4 mL). The reaction was stirred at 25 °C for 1 hour. The mixture was filtered and the filtrate was purified by reversed phase flash chromatography [C18, 0.1% formic acid condition] to afford the title compound (70 mg, 90% yield) as yellow solid; LCMS (ESI, M+1): m/z=685.2.

Step D. 2-amino-4-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl) azocan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-4-carboxylic acid (55 mg, 1 equiv), HOBt (13.0 mg, 1.2 equiv) and EDCI (23.1 mg, 1.5 equiv) in DMF (0.5 mL) were added TEA (32.5 mg, 4 equiv) and 3-methyl-1H-pyrazole (19.8 mg, 3 equiv). The reaction was stirred at 25 °C for 12 hours. The mixture was filtered and the filtrate was purified by prep-HPLC [Waters Xbridge 150×25 mm×5 μm; A: water (NH₄HCO₃), B: ACN, B %: 60%-90% over 9 min] to afford the title compound (5.75 mg, 9.1% yield) as white solid; ¹H NMR (400 MHz, CHLOROFORM-d₃) δ 8.15-8.09 (m, 1H), 7.97-7.89 (m, 1H), 7.21 (td, J=5.6, 8.4 Hz, 1H), 7.07-6.99 (m, 1H), 6.27-6.21 (m, 1H), 5.64-5.43 (m, 2H), 5.40-5.12 (m, 1H), 4.36-4.16 (m, 3H), 4.13-3.84 (m, 4H), 3.36-3.21 (m, 2H), 3.20-3.11 (m, 1H), 3.04-2.90 (m, 1H), 2.39-2.25 (m, 4H), 2.24-2.13 (m, 5H), 2.13-2.08 (m, 1H), 2.02-1.86 (m, 7H); LCMS (ESI, M+1): m/z=749.3.

### Example 138

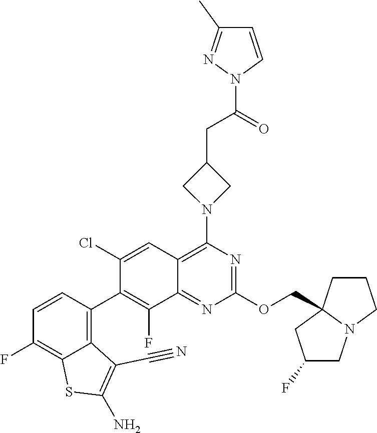 [SMILES: Cc1ccn(C(=O)CC2CN(c3nc(OC[C@@]45CCCN4C[C@H](F)C5)nc4c(F)c(-c5ccc(F)c6sc(N)c(C#N)c56)c(Cl)cc34)C2)n1]

### 2-amino-4-(6-chloro-8-fluoro-2-(((2R,7aS)-2-fluorotetrahydro-1H-pyrrolizin-7a(5H)-yl)methoxy)-4-(3-(2-(3-methyl-1H-pyrazol-1-yl)-2-oxoethyl) azetidin-1-yl)quinazolin-7-yl)-7-fluorobenzo[b]thiophene-3-carbonitrile

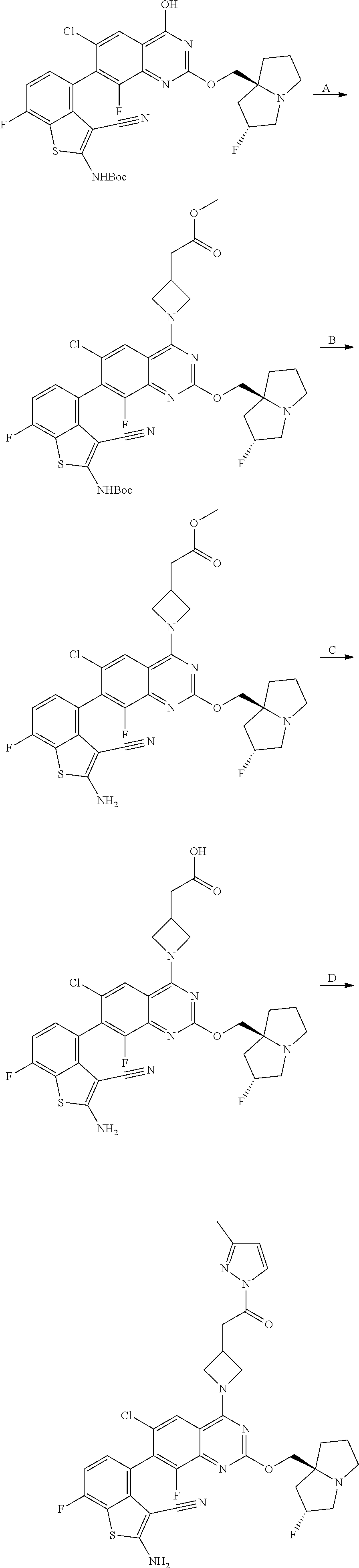

Step A. methyl 2-(1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azetidin-3-yl)acetate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (300 mg, 1.0 equiv) and methyl 2-(azetidin-3-yl)acetate (115 mg, 1.5 equiv, HCl salt) in DMF (3 mL) was added PyBOP (362 mg, 1.5 equiv) and DIEA (600 mg, 10 equiv). The reaction was stirred at 25 °C for 12 hours. The mixture was filtered and purified by reversed phase flash chromatography [C18, 0.1% formic acid condition] to afford the title compound (270 mg, 74% yield) as yellow solid; LCMS (ESI, M+1): m/z=757.4.

Step B. methyl 2-(1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azetidin-3-yl)acetate: To a solution of methyl 2-(1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azetidin-3-yl)acetate (260 mg, 1.0 equiv) in DCM (3 mL) was added TFA (2.69 mL, 105 equiv). The reaction was stirred at 25 °C for 0.5 hours. The mixture was concentrated. The residue was basified with saturated NaHCO₃ solution (20 mL) and extracted with ethyl acetate (3×20 mL). The combined organic layers were dried over anhydrous sodium sulfate, concentrated to afford the title compound (200 mg, crude) as violet solid.

Step C. 2-(1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azetidin-3-yl) acetic acid: To a solution of methyl 2-(1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azetidin-3-yl)acetate (190 mg, 1.0 equiv) in MeOH (2 mL) was added LiOH·H₂O (2 M in H₂O, 2 mL, 13.8 equiv). The reaction was stirred at 25 °C for 0.5 hours. The mixture was filtered and purified by reversed phase flash chromatography [C18, 0.1% formic acid condition] to afford the title compound (180 mg, 79% yield) as yellow solid; LCMS (ESI, M+1): m/z=643.2.

Step D. 2-amino-4-(6-chloro-8-fluoro-2-(((2R,7aS)-2-fluorotetrahydro-1H-pyrrolizin-7a(5H)-yl)methoxy)-4-(3-(2-(3-methyl-1H-pyrazol-1-yl)-2-oxoethyl) azetidin-1-yl)quinazolin-7-yl)-7-fluorobenzo[b]thiophene-3-carbonitrile: To a solution of 2-(1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azetidin-3-yl) acetic acid (100 mg, 1.0 equiv) and 3-methyl-1H-pyrazole (63.8 mg, 5 equiv) in DMF (1 mL) were added HATU (118 mg, 2.0 equiv) and DIEA (120 mg, 6.0 equiv). The reaction was stirred at 25 °C for 1 hour. The mixture was filtered and purified by reversed phase flash chromatography [C18, H₂O/MeCN condition]. The desired fractions were diluted with water (50 mL) and extracted with dichloromethane (2×30 mL). The organic layers were dried over anhydrous sodium sulfate, concentrated and purified by prep-HPLC [Waters Xbridge 150×25 mm×5 μm; A: water (NH₄HCO₃), B: ACN, B %: 48%-78% over 9 min] to afford the title compound (5.03 mg, 4.2% yield) as white solid; ¹H NMR (400 MHz, CHLOROFORM-d) δ=8.16 (d, J=2.8 Hz, 1H), 7.66 (d, J=1.4 Hz, 1H), 7.18 (dd, J=5.2, 8.4 Hz, 1H), 7.01 (t, J=8.8 Hz, 1H), 6.29 (d, J=2.8 Hz, 1H), 5.61 (br s, 2H), 5.37-5.18 (m, 1H), 4.95-4.68 (m, 2H), 4.46-4.06 (m, 4H), 3.59 (d, J=7.8 Hz, 2H), 3.47-3.33 (m, 1H), 3.31-3.11 (m, 3H), 3.02-2.90 (m, 1H), 2.34 (s, 3H), 2.29-2.10 (m, 3H), 2.00-1.82 (m, 3H); LCMS (ESI, M+1): m/z=707.2.

### Example 139

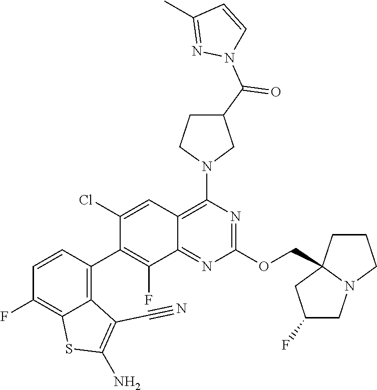 [SMILES: Cc1ccn(C(=O)C2CCN(c3nc(OC[C@@]45CCCN4C[C@H](F)C5)nc4c(F)c(-c5ccc(F)c6sc(N)c(C#N)c56)c(Cl)cc34)C2)n1]

### 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl) pyrrolidin-1-yl)quinazolin-7-yl)-7-fluorobenzo[b]thiophene-3-carbonitrile

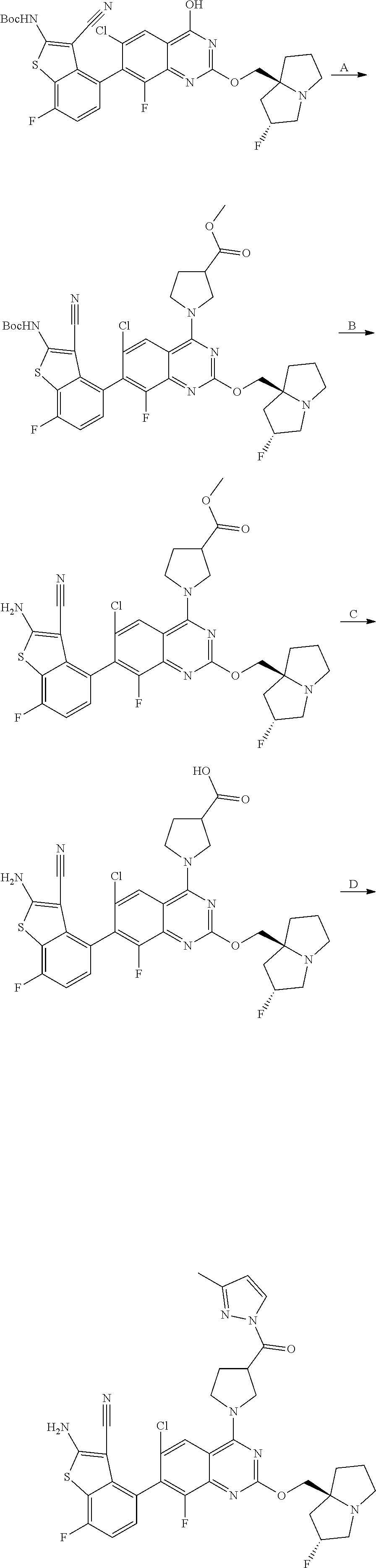

Step A. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) pyrrolidine-3-carboxylate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv) and methyl pyrrolidine-3-carboxylate (192 mg, 1.5 equiv, HCl salt) in DMF (5 mL) were added PyBOP (604 mg, 1.5 equiv) and DIEA (1.00 g, 10 equiv). The reaction was stirred at 25 °C for 1 hour. The mixture was filtered and the filtrate was purified by reversed phase flash chromatography [C18, 0.1% NH₃·H₂O condition] to afford the title compound (500 mg, 82% yield) as white solid; LCMS (ESI, M+1): m/z=757.2.

Step B. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) pyrrolidine-3-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) pyrrolidine-3-carboxylate (500 mg, 1.0 equiv) in DCM (5 mL) was added TFA (5 mL, 102 equiv). The reaction was stirred at 25 °C for 15 minutes. The mixture was concentrated. The residue was basified with saturated NaHCO₃ solution (20 mL) and extracted with ethyl acetate (3×20 mL). The combined organic layers were dried over anhydrous sodium sulfate, concentrated to afford the title compound (400 mg, crude) as violet solid.

Step C. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) pyrrolidine-3-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) pyrrolidine-3-carboxylate (400 mg, 1.0 equiv) in MeOH (4 mL) was added LiOH·H₂O (2 M in H₂O, 3.81 mL, 12 equiv). The reaction was stirred at 25 °C for 0.5 hours. The mixture was filtered and the filtrate was purified by reversed phase flash chromatography [C18, 0.1% formic acid condition] to afford the title compound (385 mg, 96% yield) as yellow solid; LCMS (ESI, M+1): m/z=643.2.

Step D. 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl) pyrrolidin-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) pyrrolidine-3-carboxylic acid (300 mg, 1.0 equiv) and 3-methyl-1H-pyrazole (191 mg, 5 equiv) in DMF (4 mL) were added HATU (355 mg, 2.0 equiv) and DIEA (362 mg, 6.0 equiv). The reaction was stirred at 25 °C for 1 hour. The mixture was filtered and purified by reversed phase flash chromatography [C18, 0.1% formic acid condition], followed by purified by prep-HPLC [Waters Xbridge 150×25 mm×5 μm; A: water (NH₄HCO₃), B: ACN, B %: 54%-84% over 9 min] to afford the title compound (14.6 mg, 4.1% yield) as yellow solid; ¹H NMR (400 MHz, CHLOROFORM-d) δ=8.19 (d, J=2.8 Hz, 1H), 8.03 (br s, 1H), 7.24-7.15 (m, 1H), 7.01 (t, J=8.6 Hz, 1H), 6.32 (d, J=2.6 Hz, 1H), 5.81-5.64 (m, 2H), 5.38-5.17 (m, 1H), 4.53-4.15 (m, 7H), 3.37-3.14 (m, 3H), 3.04-2.95 (m, 1H), 2.58-2.42 (m, 2H), 2.36 (d, J=0.8 Hz, 3H), 2.29 (br s, 1H), 2.25-2.17 (m, 2H), 1.96-1.89 (m, 3H); LCMS (ESI, M+1): m/z=707.2.

### Example 140

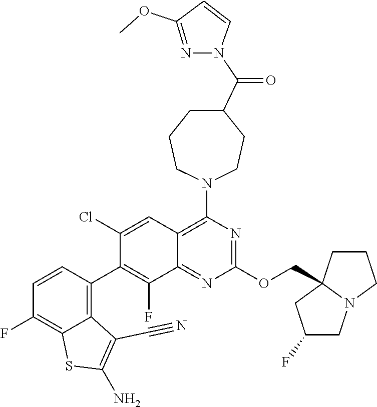 [SMILES: COc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-8-fluoro-2-(((2R,7aS)-2-fluorotetrahydro-1H-pyrrolizin-7a(5H)-yl)methoxy)-4-(4-(3-methoxy-1H-pyrazole-1-carbonyl) azepan-1-yl)quinazolin-7-yl)-7-fluorobenzo[b]thiophene-3-carbonitrile

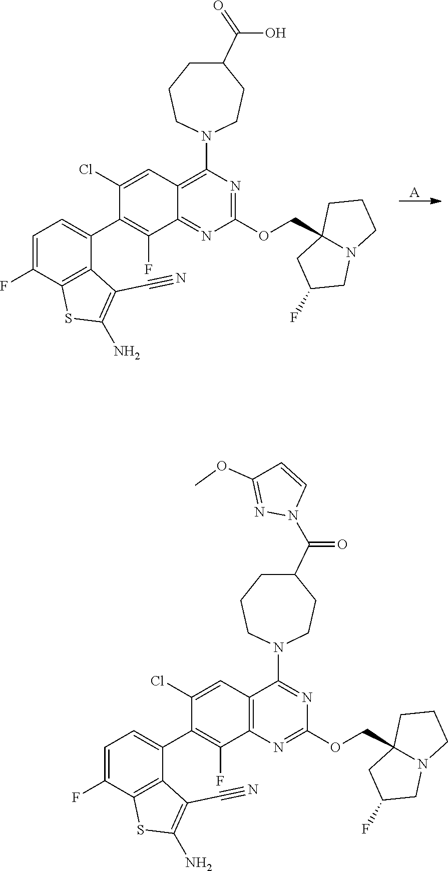

Step A. 2-amino-4-(6-chloro-8-fluoro-2-(((2R,7aS)-2-fluorotetrahydro-1H-pyrrolizin-7a(5H)-yl)methoxy)-4-(4-(3-methoxy-1H-pyrazole-1-carbonyl) azepan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (100 mg, 1.0 equiv) and 3-methoxy-1H-pyrazole (36.5 mg, 2.5 equiv) in DMAc (1 mL) were added TEA (45.2 mg, 3.0 equiv), EDCI (57.1 mg, 2.0 equiv) and HOBt (40.3 mg, 2.0 equiv). The reaction was stirred at 25 °C for 14 hours. The mixture was purified by prep-HPLC [column: Waters Xbridge 150×25 mm×10 μm; mobile phase: water (NH₄HCO₃)-acetonitrile; gradient: 66%-86% B over 8 minutes] to afford the title compound (18.6 mg, 16% yield) as white solid; ¹H NMR (400 MHz, DMSO-d₆) δ=8.24 (d, J=2.8 Hz, 1H), 8.10 (s, 2H), 8.01 (s, 1H), 7.29-7.20 (m, 1H), 7.18-7.11 (m, 1H), 6.22 (dd, J=1.6, 3.2 Hz, 1H), 5.40-5.14 (m, 1H), 4.29-3.90 (m, 5H), 3.89-3.71 (m, 4H), 3.69-3.52 (m, 1H), 3.1-2.95 (m, 3H), 2.89-2.73 (m, 1H), 2.34-2.24 (m, 1H), 2.18-1.94 (m, 7H), 1.88-1.61 (m, 4H); LCMS (ESI, M+1): m/z=751.3.

### Example 141

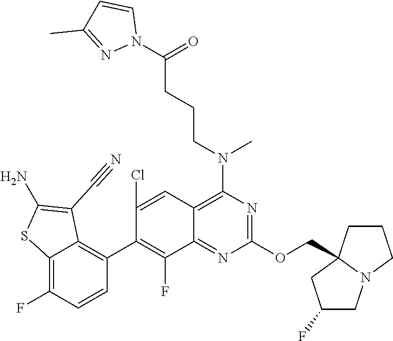 [SMILES: Cc1ccn(C(=O)CCCN(C)c2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-(methyl (4-(3-methyl-1H-pyrazol-1-yl)-4-oxobutyl)amino)quinazolin-7-yl)-7-fluorobenzo[b]thiophene-3-carbonitrile

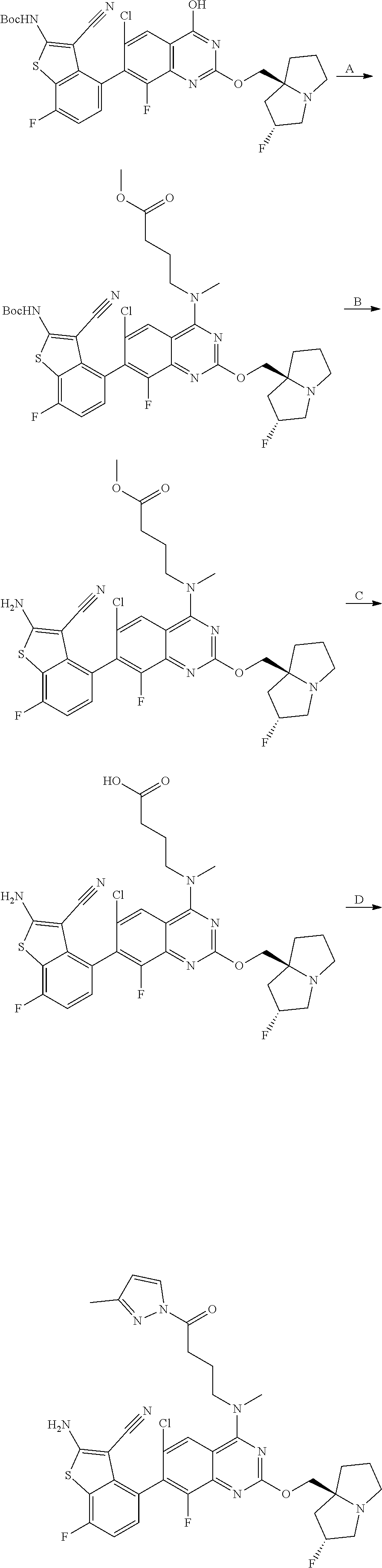

Step A. methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (800 mg, 1.0 equiv) in DMF (10 mL) were added PyBOP (966 mg, 1.5 equiv), methyl 4-(methylamino)butanoate (243 mg, 1.5 equiv, HCl) and DIEA (800 mg, 5.0 equiv). The reaction was stirred at 25 °C for 1 hour. The mixture was diluted with water (20 mL) and extracted with ethyl acetate (10 mL×3). The combined organic layers were washed with brine (20 mL), dried over anhydrous sodium sulfate, concentrated and purified by column chromatography [SiO₂, petroleum ether/ethyl acetate=1/1 to 0/1] to afford the title compound (820 mg, 86% yield) as brown solid; LCMS (ESI, M+1): m/z=759.2.

Step B. methyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate (810 mg, 1.0 equiv) in DCM (8 mL) was added TFA (2.7 mL). The reaction was stirred at 25 °C for 0.5 hours. The mixture was concentrated under reduced pressure to remove solvent. The mixture was diluted with saturated NaHCO₃ solution (20 mL) and extracted with DCM (20 mL×2). The combined organic layers were washed with brine (40 mL), dried over anhydrous sodium sulfate and concentrated to afford the title compound (630 mg, 76% yield) as brown solid; LCMS (ESI, M+1): m/z=659.2.

Step C. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid: To a solution of methyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate (620 mg, 1.0 equiv) in DMSO (3 mL) was added LiOH (3.0 mL, 5.00 equiv, 2 M). The reaction was stirred at 25 °C for 1 hour. The mixture was purified by prep-HPLC [column: Phenomenex luna C18 150×40 mm×15 μm; mobile phase: [water(FA)-ACN]; gradient: 15%-45% B over 15 min] to afford the title compound (380 mg, 62% yield) as white solid; LCMS (ESI, M+1): m/z=645.2.

Step D. 2-amino-4-(6-chloro-8-fluoro-2-(((2R,7aS)-2-fluorotetrahydro-1H-pyrrolizin-7a(5H)-yl)methoxy)-4-(methyl (4-(3-methyl-1H-pyrazol-1-yl)-4-oxobutyl)amino)quinazolin-7-yl)-7-fluorobenzo[b]thiophene-3-carbonitrile: To a solution of 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (100 mg, 1.0 equiv) in DMF (1 mL) were added HATU (88.4 mg, 1.5 equiv), 3-methyl-1H-pyrazole (19.1 mg, 1.5 equiv) and DIEA (100 mg, 5.0 equiv). The reaction was stirred at 25 °C for 1 hour. The mixture was purified by prep-HPLC [column: Waters Xbridge Prep OBD C18 150×40 mm×10 μm; mobile phase: [water (NH₄HCO₃)-ACN]; gradient: 48%-78% B over 20 min] to afford the title compound (36.9 mg, 32% yield) as off-white solid; ¹H NMR (400 MHz, CHLOROFORM-d) δ=8.14 (d, J=2.8 Hz, 1H), 7.89 (d, J=1.2 Hz, 1H), 7.20 (dd, J=5.2, 8.4 Hz, 1H), 7.02 (t, J=8.8 Hz, 1H), 6.24 (d, J=2.8 Hz, 1H), 5.70-5.56 (m, 2H), 5.36-5.18 (m, 1H), 4.27-4.20 (m, 1H), 4.17-4.10 (m, 1H), 3.98 (qd, J=7.2, 14.0 Hz, 1H), 3.85-3.73 (m, 1H), 3.45 (s, 3H), 3.29-3.20 (m, 4H), 3.16 (s, 1H), 3.01-2.93 (m, 1H), 2.31 (s, 3H), 2.30-2.11 (m, 5H), 1.97-1.85 (m, 3H); LCMS (ESI, M+1): m/z=709.4.

### Example 142

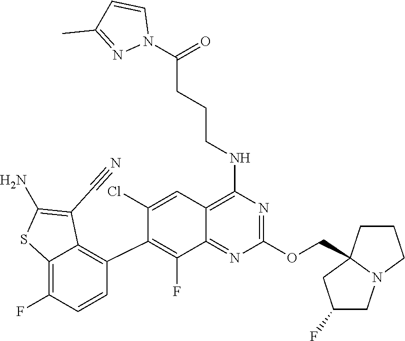 [SMILES: Cc1ccn(C(=O)CCCNc2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-((4-(3-methyl-1H-pyrazol-1-yl)-4-oxobutyl)amino)quinazolin-7-yl)-7-fluorobenzo[b]thiophene-3-carbonitrile

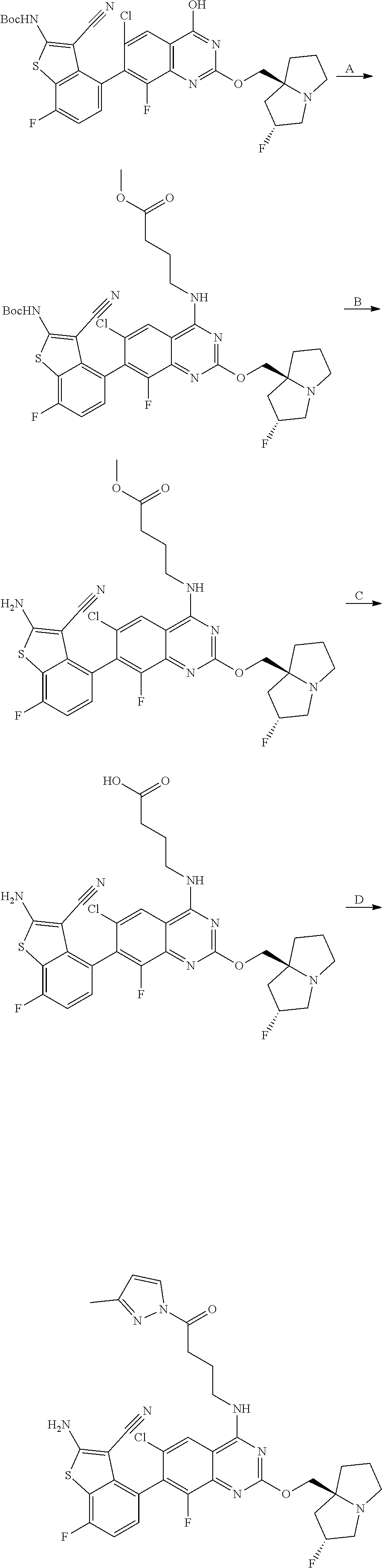

Step A. methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)butanoate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (800 mg, 1.0 equiv) in DMF (10 mL) were added PyBOP (966 mg, 1.5 equiv), methyl 4-aminobutanoate (285 mg, 1.5 equiv, HCl) and DIEA (800 mg, 5.0 equiv). The reaction was stirred at 25 °C for 1 hour. The mixture was diluted with water (20 mL) and extracted with ethyl acetate (10 mL×3). The combined organic layers were washed with brine (20 mL), dried over anhydrous sodium sulfate, concentrated and purified by column chromatography [SiO₂, Petroleum ether/Ethyl acetate=1/1 to 0/1] to afford the title compound (704 mg, 86% yield) as brown solid; LCMS (ESI, M+1): m/z=745.2.

Step B. methyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)butanoate: To a solution of methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)butanoate (694 mg, 1.0 equiv) in DCM (7 mL) was added TFA (2.4 mL). The reaction was stirred at 25 °C for 0.5 hours. The mixture was concentrated under reduced pressure to remove solvent. The mixture was diluted with saturated NaHCO₃ solution (20 mL) and extracted with DCM (20 mL×2). The combined organic layers were washed with brine (40 mL), dried over anhydrous sodium sulfate and concentrated to afford the title compound (508 mg, 75% yield) as brown solid; LCMS (ESI, M+1): m/z=645.2.

Step C. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)butanoic acid: To a solution of methyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)butanoate (498 mg, 1.0 equiv) in DMSO (2 mL) was added LiOH (2.5 mL, 5.0 equiv, 2 M). The reaction was stirred at 25 °C for 1 hour. The mixture was purified by prep-HPLC [column: Phenomenex luna C18 150×40 mm×15 μm; mobile phase: [water(FA)-ACN]; gradient: 15%-45% B over 15 min] to afford the title compound (305 mg, 61% yield) as white solid; LCMS (ESI, M+1): m/z=631.2.

Step D. 2-amino-4-(6-chloro-8-fluoro-2-(((2R,7aS)-2-fluorotetrahydro-1H-pyrrolizin-7a(5H)-yl)methoxy)-4-((4-(3-methyl-1H-pyrazol-1-yl)-4-oxobutyl)amino)quinazolin-7-yl)-7-fluorobenzo[b]thiophene-3-carbonitrile: To a solution of 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)butanoic acid (100 mg, 1.0 equiv) in DMF (1 mL) were added HATU (90.4 mg, 1.5 equiv), 3-methyl-1H-pyrazole (19.5 mg, 1.5 equiv) and DIEA (102 mg, 5.0 equiv). The reaction was stirred at 25 °C for 1 hour. The mixture was purified by prep-HPLC [column: Waters Xbridge Prep OBD C18 150×40 mm×10 μm; mobile phase: [water (NH₄HCO₃)-ACN]; gradient: 43%-73% B over 20 min] to afford the title compound (38.0 mg, 33% yield) as off-white solid; ¹H NMR (400 MHz, CHLOROFORM-d) δ=8.19 (d, J=2.8 Hz, 1H), 7.68 (s, 1H), 7.18 (dd, J=5.2, 8.4 Hz, 1H), 7.02 (t, J=8.8 Hz, 1H), 6.89-6.81 (m, 1H), 6.29 (d, J=2.8 Hz, 1H), 5.67-5.48 (m, 2H), 5.36-5.19 (m, 1H), 4.30-4.22 (m, 1H), 4.20-4.12 (m, 1H), 3.83-3.73 (m, 1H), 3.72-3.63 (m, 1H), 3.32-3.22 (m, 4H), 3.17 (s, 1H), 3.02-2.92 (m, 1H), 2.38 (s, 3H), 2.28 (br s, 1H), 2.25-2.16 (m, 4H), 1.96-1.87 (m, 3H); LCMS (ESI, M+1): m/z=695.4.

### Example 143

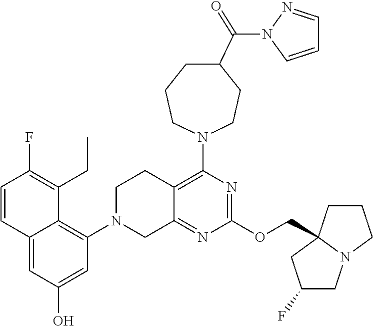 [SMILES: CCc1c(F)ccc2cc(O)cc(N3CCc4c(nc(OC[C@@]56CCCN5C[C@H](F)C6)nc4N4CCCC(C(=O)n5cccn5)CC4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azepan-4-yl)(1H-pyrazol-1-yl)methanone

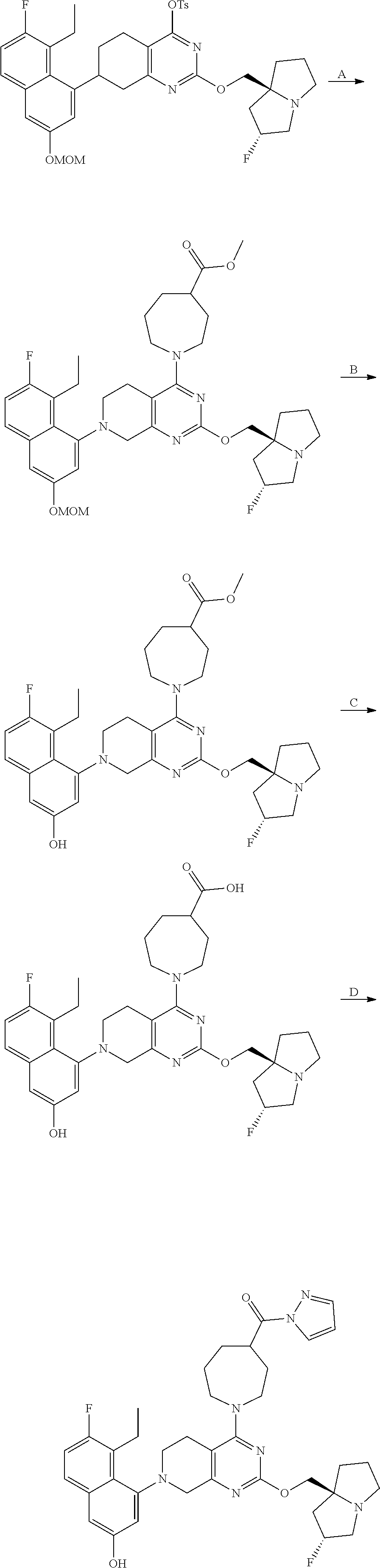

Step A. methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-4-carboxylate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (400 mg, 1.0 equiv) and DIEA (223 mg, 3.0 equiv) in DMAc (1.0 mL) was added methyl azepane-4-carboxylate hydrogen chloride (167 mg, 1.5 equiv). The reaction was stirred at 40 °C for 12 hours. The mixture was diluted with water (20 mL) and extracted with ethyl acetate (4×10 mL). The combined organic layers were washed with brine (10 mL), dried over sodium sulfate and concentrated to afford the title compound (500 mg, crude) as brown solid; LCMS (ESI, M+1): m/z=680.4.

Step B. methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-4-carboxylate: To a solution of methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-4-carboxylate (500 mg, 1.0 equiv) in MeOH (2.0 mL) was added HCl·MeOH (4 M, 4.0 mL) at 0 °C. The reaction was stirred at 0 °C for 0.5 hours. The mixture was concentrated. The residue was dissolved in ethyl acetate (10 mL) and saturated NaHCO₃ aqueous (10 mL) at 0 °C. The mixture was extracted with ethyl acetate (4×10 mL). The combined organic layers were washed brine (20 mL), dried over anhydrous sodium sulfate and concentrated to afford the title compound (364 mg, crude) as brown foam; LCMS (ESI, M+1): m/z=636.4.

Step C. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-4-carboxylic acid: To a solution of methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-4-carboxylate (340 mg, 1.0 equiv) in MeOH (1 mL) was added LiOH (1.5 M in H₂O, 1.1 mL, 3.0 equiv). The reaction was stirred at 20 °C for 2 hours. The pH of the mixture was adjusted to 8 with 1N HCl at 0 °C. The mixture was purified by reversed phase flash [0.1% FA condition] to afford the title compound (260 mg, 73% yield over three steps) as white solid; LCMS (ESI, M+1): m/z=622.4.

Step D. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azepan-4-yl)(1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-4-carboxylic acid (140 mg, 1.0 equiv) and 1H-pyrazole (77 mg, 5.0 equiv) in DMAc (1.0 mL) were added EDCI (65 mg, 1.5 equiv), HOBt (46 mg, 1.5 equiv) and TEA (182 mg, 8.0 equiv). The reaction was stirred at 40 °C for 2 hours. The mixture was purified by prep-HPLC [column: Waters Xbridge 150×25 mm×5 μm; mobile phase: water (NH₄HCO₃)-ACN; gradient: 57%-77% B over 53 minutes] to afford the title compound (57.6 mg, 37% yield) as white solid; ¹H NMR (400 MHz, CD₃OD) δ=8.35-8.26 (m, 1H), 7.70-7.60 (m, 1H), 7.50 (dd, J=6.0, 9.2 Hz, 1H), 7.13 (t, J=9.2 Hz, 1H), 7.04-6.92 (m, 2H), 6.54 (dt, J=1.6, 2.8 Hz, 1H), 5.36-5.13 (m, 1H), 4.24-3.96 (m, 5H), 3.96-3.58 (m, 4H), 3.55-3.45 (m, 1H), 3.45-3.33 (m, 2H), 3.29-3.07 (m, 5H), 3.03-2.90 (m, 1H), 2.80-2.71 (m, 1H), 2.40-1.68 (m, 12H), 1.10 (q, J=7.6 Hz, 3H); LCMS (ESI, M+1): m/z=672.4.

### Example 144

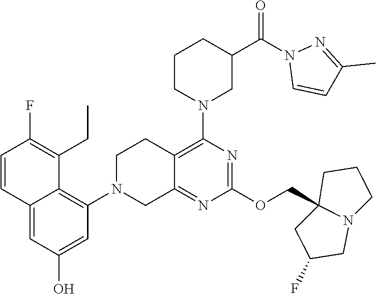 [SMILES: CCc1c(F)ccc2cc(O)cc(N3CCc4c(nc(OC[C@@]56CCCN5C[C@H](F)C6)nc4N4CCCC(C(=O)n5ccc(C)n5)C4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidin-3-yl)(3-methyl-1H-pyrazol-1-yl)methanone

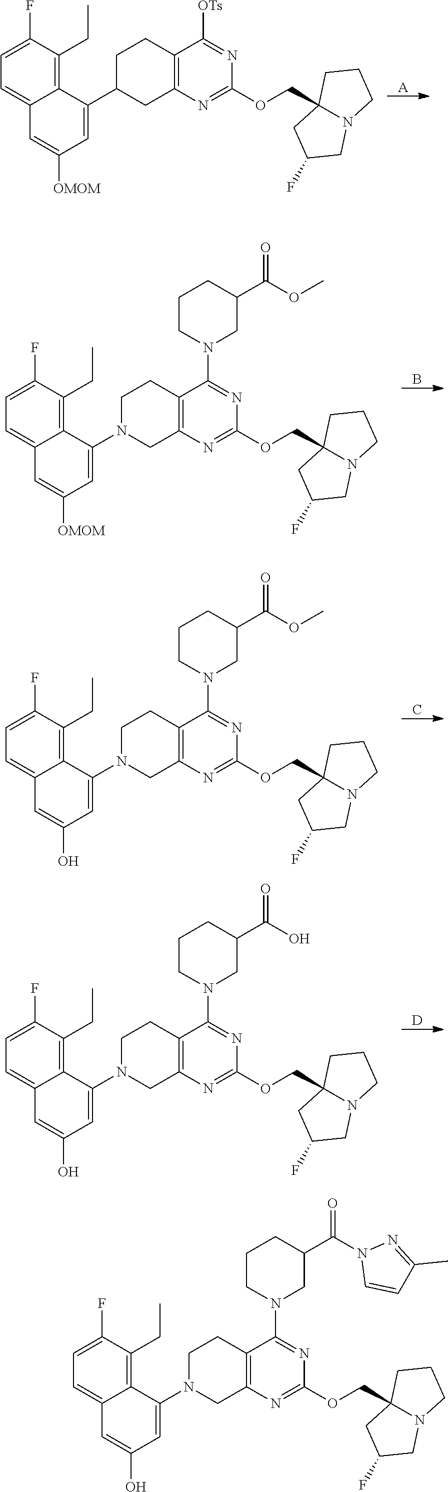

Step A. methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-3-carboxylate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (0.4 g, 1.0 equiv) and methyl piperidine-3-carboxylate (140 mg, 1.7 equiv) in DMAc (1.4 mL) was added DIPEA (186 mg, 2.5 equiv). The reaction was stirred at 40 °C for 13 hours. The mixture was diluted with H₂O (7 mL) and extracted with ethyl acetate (6×3 mL). The combined organic layers were washed with brine (5 mL), dried over anhydrous sodium sulfate, and concentrated to afford the title compound (0.45 g, crude) as red solid; LCMS (ESI, M+1): m/z=666.4.

Step B. methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-3-carboxylate: To a solution of methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-3-carboxylate (450 mg, 1.0 equiv) in MeOH (2.0 mL) was added HCl·MeOH (4M, 4.0 mL) dropwise at 0 °C. The reaction was stirred at 0 °C for 0.5 hours. The mixture was concentrated. The residue was dissolved in ethyl acetate (10 mL) and saturated NaHCO₃ aqueous (10 mL) at 0 °C. The mixture was extracted with ethyl acetate (4×10 mL). The combined organic layers were washed brine (8 mL), dried over anhydrous sodium sulfate, and concentrated to afford the title compound (0.4 g, crude) as red solid; LCMS (ESI, M+1): m/z=622.3

Step C. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-3-carboxylic acid: To a solution of methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-3-carboxylate (400 mg, 1.0 equiv) in MeOH (1.2 mL) was added LiOH·H₂O (1.5 M in H₂O, 1.3 mL, 3.0 equiv). The reaction was stirred at 20 °C for 2 hours. The pH of the mixture was adjusted to 8 with 1N HCl at 0 °C. The mixture was purified by reversed phase flash [water(FA, 0.1%)/acetonitrile=13/7] to afford the title compound (0.3 g, 85% yield over three steps) as light yellow solid; LCMS (ESI, M+1): m/z=608.3

Step D. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidin-3-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-3-carboxylic acid (100 mg, 1.0 equiv) and 3-methyl-1H-pyrazole (34 mg, 2.5 equiv) in DMAC (1.0 mL) were added TEA (72.7 mg, 4.5 equiv), HOBt (42.3 mg, 1.9 equiv) and EDCI (56.8 mg, 1.8 equiv). The reaction was stirred at 20 °C for 14 hours. The mixture was purified by prep-HPLC [column: Waters Xbridge 150×25 mm×5 μm; mobile phase: water (NH₄HCO₃)-ACN; gradient: 62%-82% B over 53 minutes] to afford the title compound (19.8 mg, 17% yield) as white solid; ¹H NMR (400 MHz, DMSO) δ=9.68 (d, J=6.4 Hz, 1H), 8.30 (t, J=3.2 Hz, 1H), 7.59 (dd, J=6.0, 8.8 Hz, 1H), 7.24 (t, J=9.2 Hz, 1H), 6.98 (dd, J=2.4, 4.8 Hz, 2H), 6.46 (t, J=2.4 Hz, 1H), 5.30-5.10 (m, 1H), 4.20-4.05 (m, 1H), 4.00-3.72 (m, 5H), 3.70-3.60 (m, 1H), 3.26-2.86 (m, 8H), 2.83-2.73 (m, 1H), 2.70-2.53 (m, 1H), 2.46-2.34 (m, 1H), 2.21 (d, J=12.0 Hz, 3H), 2.13-1.57 (m, 11H), 1.01 (td, J=7.2, 10.0 Hz, 3H); F NMR (376 MHz, DMSO) δ=−121, −172; LCMS (ESI, M+1): m/z=672.3.

### Example 145

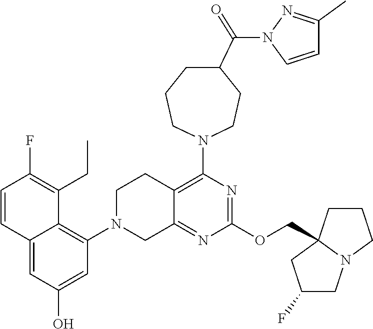 [SMILES: CCc1c(F)ccc2cc(O)cc(N3CCc4c(nc(OC[C@@]56CCCN5C[C@H](F)C6)nc4N4CCCC(C(=O)n5ccc(C)n5)CC4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azepan-4-yl)(3-methyl-1H-pyrazol-1-yl)methanone

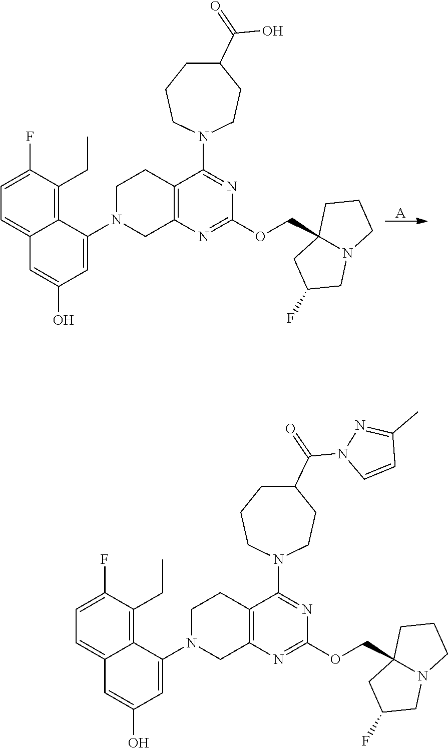

Step A. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azepan-4-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-4-carboxylic acid (90 mg, 1.0 equiv) and 3-methyl-1H-pyrazole (59 mg, 5.0 equiv) in DMAc (1 mL) were added EDCI (42 mg, 1.5 equiv), HOBt (29 mg, 1.5 equiv) and TEA (117 mg, 8.0 equiv). The reaction was stirred at 40 °C for 2 hours. The mixture was purified by prep-HPLC [column: Waters Xbridge 150×25 mm×5 μm; mobile phase: water (NH₄HCO₃)-ACN; gradient: 57%-77% B over 53 minutes] to afford the title compound (30.1 mg, 30% yield) as white solid; ¹H NMR (400 MHz, CD₃OD) δ=8.23-8.13 (m, 1H), 7.50 (dd, J=6.0, 9.2 Hz, 1H), 7.13 (t, J=9.2 Hz, 1H), 7.02-6.94 (m, 2H), 6.40-6.34 (m, 1H), 5.38-5.13 (m, 1H), 4.25-3.96 (m, 5H), 3.94-3.59 (m, 4H), 3.55-3.34 (m, 3H), 3.29-3.09 (m, 5H), 3.04-2.91 (m, 1H), 2.85-2.75 (m, 1H), 2.30 (s, 3H), 2.28-1.67 (m, 12H), 1.09 (td, J=7.2, 10.4 Hz, 3H); LCMS (ESI, M+1): m/z=686.4.

### Example 146

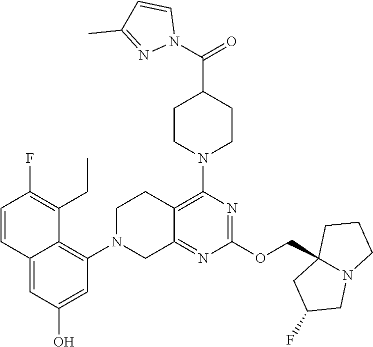 [SMILES: CCc1c(F)ccc2cc(O)cc(N3CCc4c(nc(OC[C@@]56CCCN5C[C@H](F)C6)nc4N4CCC(C(=O)n5ccc(C)n5)CC4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidin-4-yl)(3-methyl-1H-pyrazol-1-yl)methanone

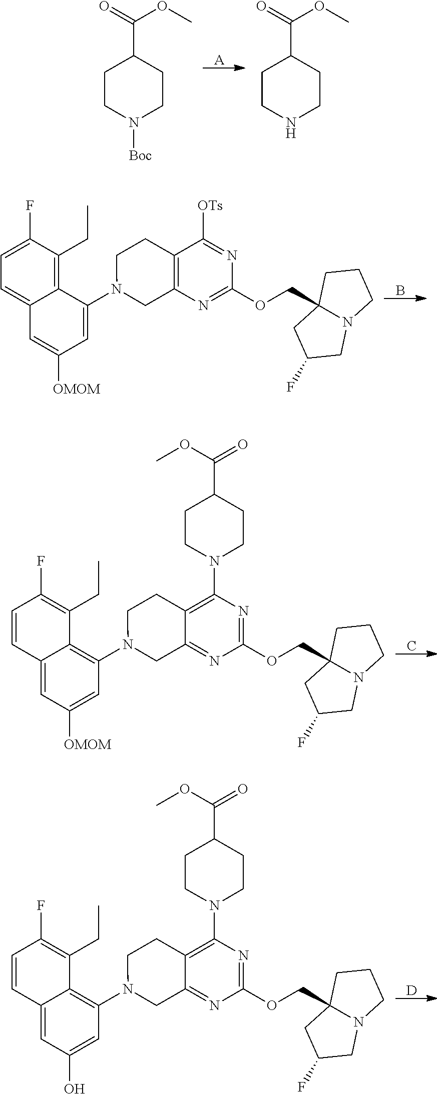

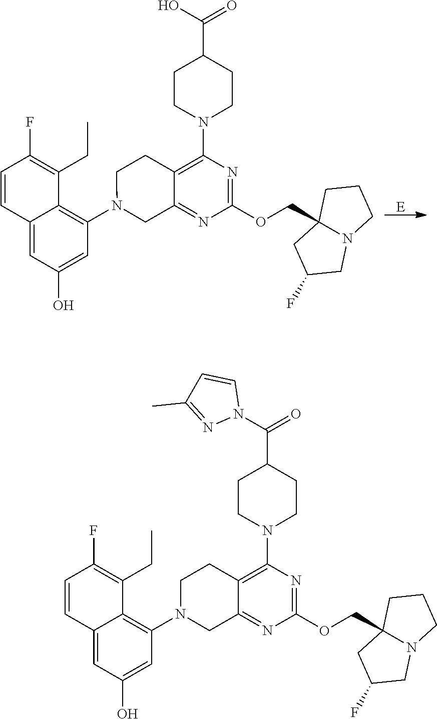

Step A. methyl piperidine-4-carboxylate: To a solution of 1-(tert-butyl) 4-methyl piperidine-1,4-dicarboxylate (1.00 g, 1.0 equiv) in DCM (10 mL) was added TFA (5.0 mL, 16 equiv) at 0 °C. The reaction was stirred at 0 °C for 1 hour. The mixture was concentrated to afford the title compound (500 mg, crude) as a colorless oil; LCMS (ESI, M+1): m/z=144.0.

Step B. methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-4-carboxylate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (600 mg, 1.0 equiv) and DIEA (335 mg, 3.0 equiv) in DMF (8.0 mL) were added methyl piperidine-4-carboxylate (247 mg, 2.0 equiv) and 4 Å molecular sieve (10.0 mg). The reaction was stirred at 40 °C for 12 hours. The mixture was diluted by water (10 mL) and extracted with ethyl acetate (3×10 mL). The combined organic layers were dried over anhydrous sodium sulfate, concentrated and purified by reversed phase flash chromatography [C18, 0.1% formic acid condition] to afford the title compound (352 mg, 27% yield) as yellow oil; LCMS (ESI, M+1): m/z=666.4.

Step C. methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-4-carboxylate: A mixture of methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-4-carboxylate (352 mg, 1.0 equiv) and HCl/MeOH (2 M, 5.0 mL, 19 equiv) was stirred at 25 °C for 1 hour. The mixture was concentrated to afford the title compound (320 mg, crude) yellow oil; LCMS (ESI, M+1): m/z=622.6.

Step D. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-4-carboxylic acid: To a solution of methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-4-carboxylate (320 mg, 1.0 equiv) in THF (1.5 mL) was added LiOH (2 M, 0.772 μL, 3.0 equiv). The reaction was stirred at 25 °C for 5 hours. The mixture was concentrated and purified by reversed phase flash chromatography [C18, 0.1% formic acid condition] to afford the title compound (300 mg, 86% yield) as yellow solid; LCMS (ESI, M+1): m/z=608.4.

Step E. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidin-4-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)piperidine-4-carboxylic acid (100 mg, 1.0 equiv) and DIEA (21.3 mg, 3.0 equiv) in DMF (1.5 mL) were added EDCI (94.6 mg, 3.0 equiv) and HOBt (66.7 mg, 3.0 equiv). After stirred at 25 °C for 0.5 hours, 3-methyl-1H-pyrazole (20.3 mg, 1.5 equiv) was added to the mixture. The reaction was stirred at 25 °C for 1 hour. The mixture was quenched with water (5 mL) at 0 °C and extracted with ethyl acetate (3×10 mL). The combined organic phase was dried over anhydrous sodium sulfate, concentrated and purified by reversed phase flash chromatography [C18, 0.1% formic acid condition] to afford the title compound (29.1 mg, 24% yield) as yellow solid; ¹H NMR (400 MHz, CHLOROFORM-d) δ=8.16 (d, J=2.4 Hz, 1H), 7.43 (ddd, J=2.4, 6.0, 8.7 Hz, 1H), 7.13 (dt, J=3.8, 9.2 Hz, 1H), 6.93 (br s, 2H), 6.28 (d, J=2.8 Hz, 1H), 5.37-5.13 (m, 1H), 4.16 (br d, J=9.0 Hz, 2H), 4.12 (br d, J=4.4 Hz, 2H), 3.95-3.82 (m, 1H), 3.75-3.61 (m, 1H), 3.37 (br d, J=2.8 Hz, 2H), 3.34-3.25 (m, 2H), 3.24-3.11 (m, 3H), 3.10-2.91 (m, 4H), 2.60-2.47 (m, 1H), 2.36 (s, 3H), 2.32-2.16 (m, 3H), 2.04 (br s, 5H), 1.96-1.91 (m, 2H), 1.87 (br dd, J=3.4, 12.1 Hz, 2H), 1.09 (br t, J=7.0 Hz, 3H); LCMS (ESI, M+1): m/z=672.1.

### Example 147

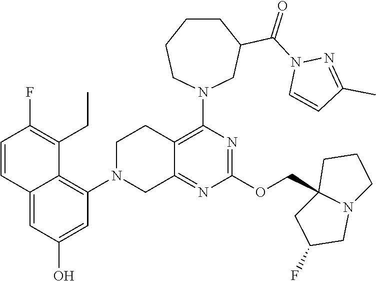 [SMILES: CCc1c(F)ccc2cc(O)cc(N3CCc4c(nc(OC[C@@]56CCCN5C[C@H](F)C6)nc4N4CCCCC(C(=O)n5ccc(C)n5)C4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azepan-3-yl)(3-methyl-1H-pyrazol-1-yl)methanone

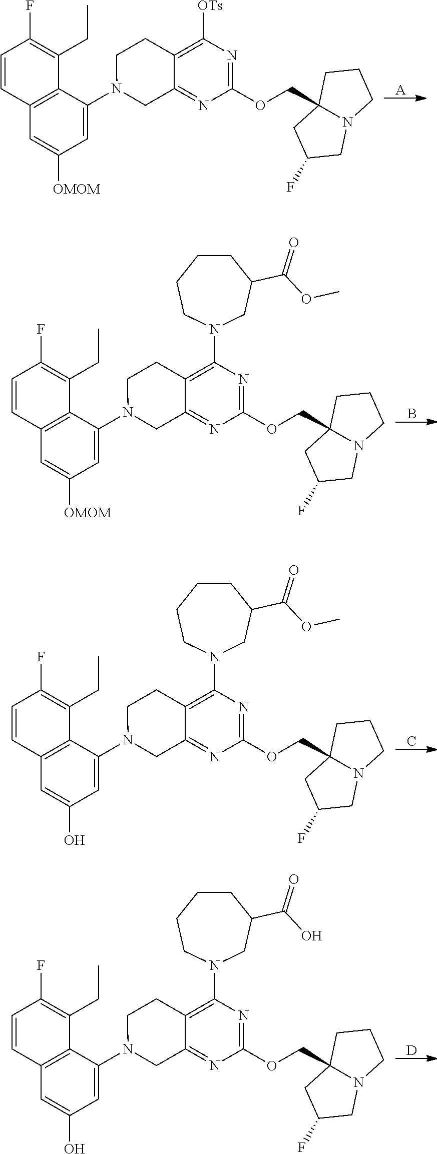

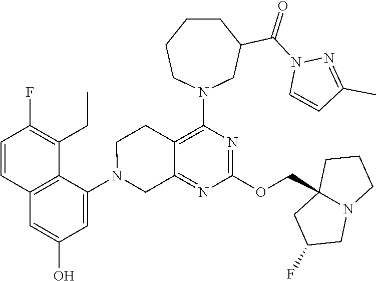 [SMILES: CCc1c(F)ccc2cc(O)cc(N3CCc4c(nc(OC[C@@]56CCCN5C[C@H](F)C6)nc4N4CCCCC(C(=O)n5ccc(C)n5)C4)C3)c12]

Step A. methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-3-carboxylate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (300 mg, 1.0 equiv) and DIEA (224 mg, 4.0 equiv) in DMAc (0.3 mL) was added and methyl azepane-3-carboxylate hydrogen chloride (101 mg, 1.2 equiv). The reaction was stirred at 35 °C for 14 hours. The mixture was purified by reversed phase flash chromatography [water (0.1% formic acid)/acetonitrile=3/2] to afford the title compound (195 mg, 66% yield) as brown oil; LCMS (ESI, M+1): m/z=680.3.

Step B. methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-3-carboxylate: To a solution of methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-3-carboxylate (193 mg, 1.0 equiv) in MeOH (2 mL) was added HCl·MeOH (2 M, 3 mL) at 0 °C. The reaction was stirred at 0 °C for 1 hour. The mixture was concentrated. The residue was dissolved in ethyl acetate (10 mL) and saturated NaHCO₃ aqueous (10 mL) at 0 °C. The mixture extracted with ethyl acetate (10 mL×4). The combined organic layers were washed with brine (10 mL), dried over anhydrous sodium sulfate and concentrated to afford the title compound (177 mg, 95% yield) as brown solid; LCMS (ESI, M+1): m/z=636.4.

Step C. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-3-carboxylic acid: To a solution of methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-3-carboxylate (170 mg, 1.0 equiv) in MeOH (1.7 mL) was added LiOH·H₂O (1.5 M, 534 μL, 3.0 equiv). The reaction was stirred at 20 °C for 1 hour. The pH of the mixture was adjusted to 8 with 1N HCl at 0 °C. The mixture was purified by reversed phase flash chromatography [water (0.1% formic acid)/acetonitrile=2/1] to afford the title compound (125 mg, 75% yield) as pink solid; LCMS (ESI, M+1): m/z=622.3.

Step D. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azepan-3-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azepane-3-carboxylic acid (100 mg, 1.0 equiv) and 3-methyl-1H-pyrazole (33.0 mg, 2.5 equiv) in DMAc (1 mL) were added TEA (73.2 mg, 4.5 equiv), HOBt (43.5 mg, 2.0 equiv) and EDCI (61.7 mg, 2.0 equiv). The reaction was stirred at 20 °C for 14 hours. The mixture was purified by prep-HPLC [column: Waters Xbridge 150×25 mm×5 μm; mobile phase: water (NH₄HCO₃)-acetonitrile; gradient: 54%-74% B over 10 minutes] to afford the title compound (46.0 mg, 40% yield) as white solid; ¹H NMR (400 MHz, DMSO-d₆) δ=9.76-9.65 (m, 1H), 8.34-8.26 (m, 1H), 7.62-7.54 (m, 1H), 7.23 (dt, J=4.0, 9.2 Hz, 1H), 7.03-6.94 (m, 2H), 6.50-6.43 (m, 1H), 5.33-5.06 (m, 1H), 4.22 (br d, J=10.8 Hz, 1H), 4.12-3.94 (m, 1H), 3.94-3.81 (m, 2H), 3.80-3.53 (m, 4H), 3.49-3.41 (m, 1H), 3.29-3.21 (m, 2H), 3.21-2.60 (m, 8H), 2.23 (d, J=7.2 Hz, 3H), 2.00-1.40 (m, 12H), 1.11-0.94 (m, 3H); LCMS (ESI, M+1): m/z=686.4.

### Example 148

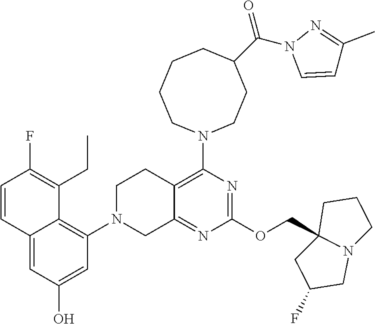 [SMILES: CCc1c(F)ccc2cc(O)cc(N3CCc4c(nc(OC[C@@]56CCCN5C[C@H](F)C6)nc4N4CCCCC(C(=O)n5ccc(C)n5)CC4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azocan-4-yl)(3-methyl-1H-pyrazol-1-yl)methanone

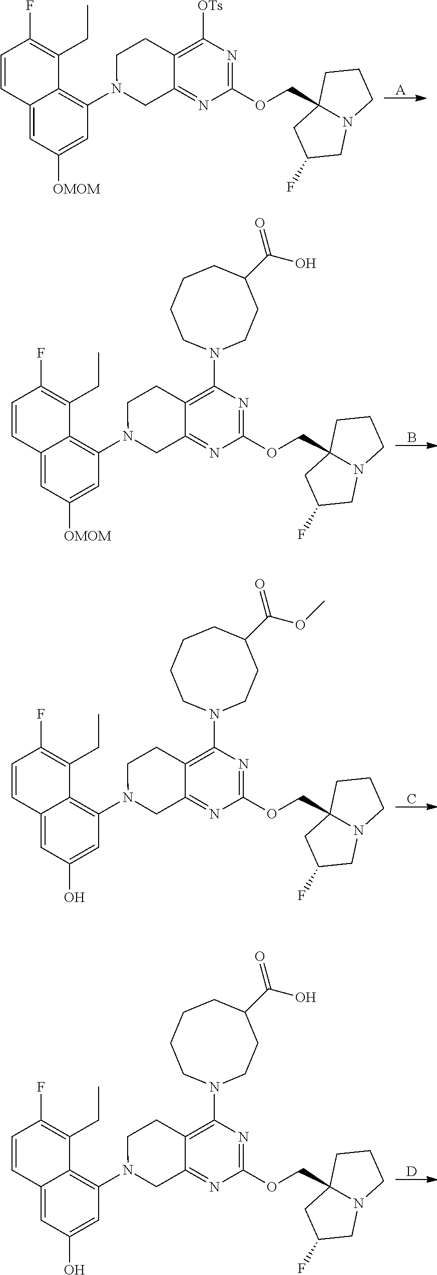

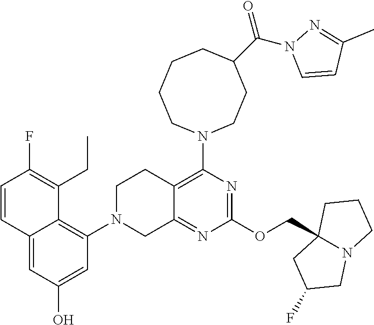 [SMILES: CCc1c(F)ccc2cc(O)cc(N3CCc4c(nc(OC[C@@]56CCCN5C[C@H](F)C6)nc4N4CCCCC(C(=O)n5ccc(C)n5)CC4)C3)c12]

Step A. 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-4-carboxylic acid: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (230 mg, 1.0 equiv) and azocane-4-carboxylic acid (147 mg, 2.3 equiv, HCl salt) in DMF (1 mL) were added 4 Å molecular sieve (20 mg) and DIEA (257 mg, 6.0 equiv). The reaction was stirred at 60 °C for 5 hours. The mixture was filtered and purified by reversed phase flash chromatography [water (0.1% FA)/acetonitrile] to afford the title compound (160 mg, 62% yield, HCOOH salt) as yellow solid; LCMS (ESI, M+1): m/z=680.3.

Step B. methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-4-carboxylate: To a solution of 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-4-carboxylic acid (160 mg, 1.0 equiv) in MeOH (2 mL) was added HCl·MeOH (2 M, 7.85 mL, 66 equiv). The reaction was stirred at 25 °C for 1 hour. The mixture was concentrated. The residue was purified by reversed phase flash chromatography [C18, 0.1% formic acid condition] to afford the title compound (95 mg, 61% yield) as yellow solid; ¹H NMR (400 MHz, CHLOROFORM-d₃) δ 7.48-7.38 (m, 1H), 7.18-7.07 (m, 1H), 7.03-6.85 (m, 2H), 5.37-5.13 (m, 1H), 4.28-3.99 (m, 3H), 3.95-3.73 (m, 3H), 3.69 (s, 3H), 3.68-3.54 (m, 2H), 3.43-3.18 (m, 5H), 3.18-2.93 (m, 4H), 2.67-2.49 (m, 2H), 2.34-2.07 (m, 4H), 2.03-1.83 (m, 6H), 1.79-1.70 (m, 4H), 1.14-1.05 (m, 3H); LCMS (ESI, M+1): m/z=650.4.

Step C. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-4-carboxylic acid: To a solution of methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-4-carboxylate (85 mg, 1.0 equiv) in EtOH (1.5 mL) was added a solution of NaOH (26.2 mg, 5.0 equiv) in H₂O (0.5 mL). The reaction was stirred at 25 °C for 1 hour. The mixture was filtered and the filtrate was purified by reversed phase flash chromatography [C18, 0.1% formic acid condition] to afford the title compound (80 mg, 90% yield, HCOOH salt) as yellow solid; LCMS (ESI, M+1): m/z=636.5.

Step D. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azocan-4-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-4-carboxylic acid (40 mg, 1 equiv) and HATU (35.9 mg, 1.5 equiv) in DMF (0.4 mL) were added 3-methyl-1H-pyrazole (15.5 mg, 3 equiv) and DIEA (24.4 mg, 3 equiv). The reaction was stirred at 25 °C for 1 hour. The mixture was filtered and purified by prep-HPLC [Waters Xbridge 150×25 mm×5 μm; A: water (NH₄HCO₃), B: ACN, B %: 58%-88% over 9 min] to afford the title compound (14.7 mg, 33% yield) as yellow solid; ¹H NMR (400 MHz, CHLOROFORM-d₃) δ 8.16-8.10 (m, 1H), 7.40 (ddd, J=2.4, 5.6, 8.8 Hz, 1H), 7.14-7.04 (m, 1H), 7.00-6.85 (m, 2H), 6.29-6.22 (m, 1H), 5.34-5.13 (m, 1H), 4.10-3.87 (m, 5H), 3.86-3.59 (m, 3H), 3.42-3.10 (m, 6H), 3.09-2.90 (m, 3H), 2.69-2.53 (m, 1H), 2.33-2.28 (m, 3H), 2.27-2.21 (m, 1H), 2.20-2.14 (m, 1H), 2.14-2.06 (m, 3H), 1.97-1.76 (m, 10H), 1.14-1.02 (m, 3H); LCMS (ESI, M+1): m/z=700.4.

### Example 149

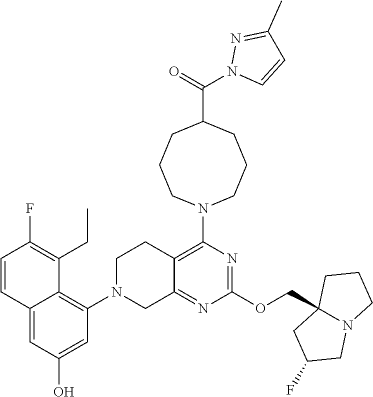 [SMILES: CCc1c(F)ccc2cc(O)cc(N3CCc4c(nc(OC[C@@]56CCCN5C[C@H](F)C6)nc4N4CCCC(C(=O)n5ccc(C)n5)CCC4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azocan-5-yl)(3-methyl-1H-pyrazol-1-yl)methanone

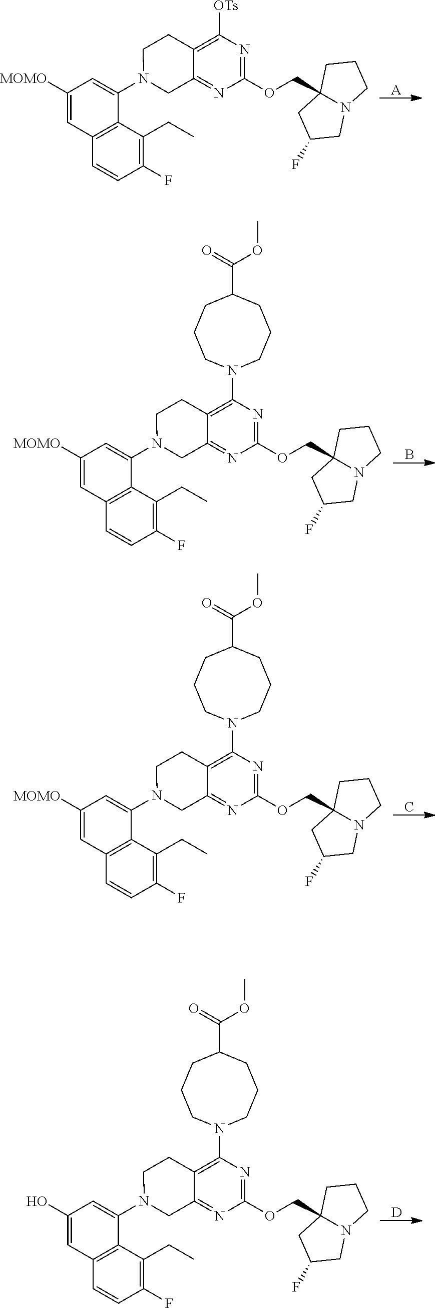

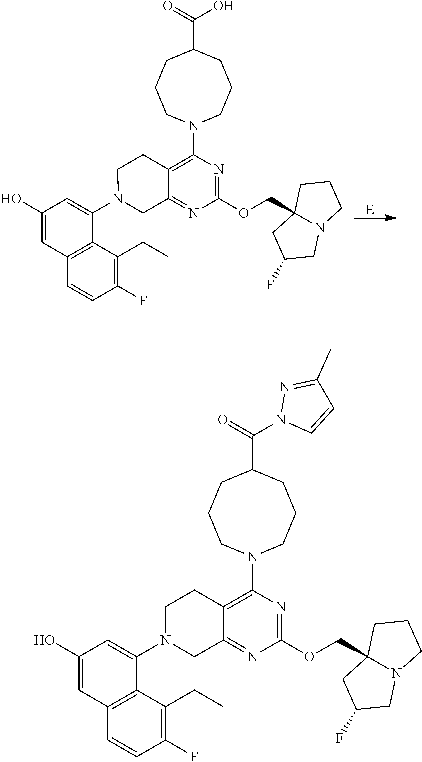

Step A. methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (300 mg, 1.0 equiv), methyl azocane-5-carboxylate (224 mg, 2.5 equiv, HCl) in DMF (1 mL) were added 4 Å molecular sieve (50 mg) and DIEA (279 mg, 5.0 equiv). The reaction was stirred at 60 °C for 12 hours. The mixture was diluted with H₂O (20 mL) and extracted with ethyl acetate (3×10 mL). The combined organic layers were washed with brine (10 mL), dried over anhydrous sodium sulfate, concentrated and purified by column chromatography (SiO₂, petroleum ether/ethyl acetate=10/1 to ethyl acetate/methanol=10/1) to afford the title compound (300 mg, 98% yield) as yellow solid; LCMS (ESI, M+1): m/z=694.5

Step B. 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylic acid: To a solution of methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylate (300 mg, 1.0 equiv) in MeOH (3 mL), THF (1 mL) and H₂O (1 mL) was added LiOH·H₂O (544 mg, 30 equiv). The reaction was stirred at 25 °C for 2 hours. The mixture was diluted with H₂O (10 mL) and extracted with ethyl acetate (3×5 mL). The combined organic layers were washed with brine (10 mL), dried over anhydrous sodium sulfate, concentrated and purified by reversed phase flash chromatography [C18, 0.1% formic acid condition] to afford the title compound (290 mg, 89% yield, HCOOH) as yellow solid; LCMS (ESI, M+1): m/z=680.5.

Step C. methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylate: A mixture of methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylate (120 mg, 1.0 equiv) in HCl·MeOH (4 M, 2 mL) was stirred at 0 °C for 1 hour. The mixture was concentrated, diluted with H₂O (10 mL), adjusted to pH about 8 with NaHCO₃solid and extracted with ethyl acetate (3×5 mL). The combined organic layers were washed with brine (10 mL), dried over anhydrous sodium sulfate, concentrated to afford the title compound (100 mg, 87% yield, crude) as yellow solid.

Step D. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylic acid: To a solution of methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylate (100 mg, 1.0 equiv) in MeOH (3 mL), THF (1 mL) and H₂O (1 mL) was added LiOH·H₂O (480 mg, 71 equiv). The reaction was stirred at 25 °C for 2 hours. The mixture was diluted with H₂O (10 mL) and extracted with ethyl acetate (3×5 mL). The combined organic layers were washed with brine (10 mL), dried over anhydrous sodium sulfate, concentrated and purified by reversed phase flash chromatography [C18, 0.1% formic acid condition] to afford the title compound (80.0 mg, 82%) as yellow solid; LCMS (ESI, M+1): m/z=636.4.

Step E. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azocan-5-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-5-carboxylic acid (80.0 mg, 1.0 equiv) and EDCI (36.2 mg, 1.5 equiv) in DMF (2 mL) were added 3-methyl-1H-pyrazole (20.7 mg, 2.0 equiv) and DMAP (23.1 mg, 1.5 equiv). The reaction was stirred at 25 °C for 2 hours. The mixture was filtered and purified by prep-HPLC [Waters Xbridge 150×25 mm×5 μm; A: water (NH₄HCO₃), B: ACN, B %: 62%-92% over 9 min] to afford the title compound (5.59 mg, 6.2% yield) as yellow solid; ¹H NMR (400 MHz, CHLOROFORM-d₃) δ=8.13 (d, J=2.4 Hz, 1H), 7.44-7.36 (m, 1H), 7.09 (br t, J=9.2 Hz, 1H), 6.99-6.90 (m, 2H), 6.25 (d, J=2.8 Hz, 1H), 5.35-5.15 (m, 1H), 4.10-3.88 (m, 4H), 3.80-3.53 (m, 3H), 3.41-3.30 (m, 4H), 3.28-3.10 (m, 2H), 3.09-2.91 (m, 3H), 2.58 (br t, J=13.2 Hz, 1H), 2.48-2.33 (m, 2H), 2.31 (s, 3H), 2.29-2.22 (m, 3H), 2.21-2.16 (m, 3H), 2.15-2.06 (m, 5H), 1.89-1.76 (m, 3H), 1.08 (t, J=7.2 Hz, 3H); LCMS (ESI, M+1): m/z=700.5.

### Example 150

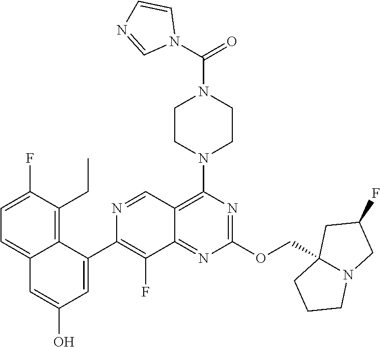 [SMILES: CCc1c(F)ccc2cc(O)cc(-c3ncc4c(N5CCN(C(=O)n6ccnc6)CC5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(1H-imidazol-1-yl)methanone

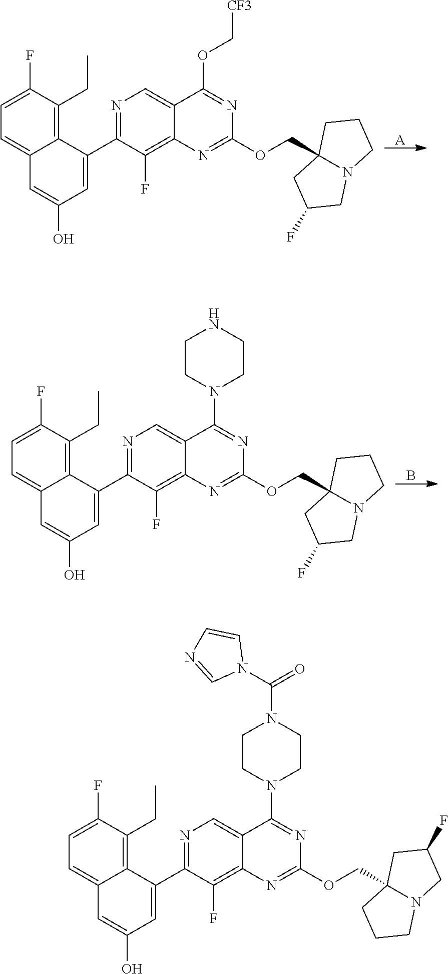

Step A. 5-ethyl-6-fluoro-4-(8-fluoro-2-(((2R,7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol: To a solution of piperazine (726 mg, 5.0 equiv) in DMF (10 mL) was added 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (1.00 g, 1.0 equiv) at 25 °C. The reaction was stirred at 25 °C for 2 hours. The mixture was purified with reversed phase flash [water/acetonitrile=1/1] to afford the title compound (914 mg, 90% yield) as yellow solid; LCMS (ESI, M+1): m/z=579.4.

Step B. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(1H-imidazol-1-yl)methanone: To a solution of 5-ethyl-6-fluoro-4-(8-fluoro-2-(((2R,7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (100 mg, 1.0 equiv) in DMF (1.1 mL) was added CDI (30.8 mg, 1.1 equiv) at 0 °C. The reaction was stirred at 25 °C for 0.25 hours. The mixture was purified with reversed phase flash [water/acetonitrile=1/1] and prep-HPLC [column: Waters xbridge 150×25 mm×10 μm; mobile phase: (water (NH₄HCO₃)-acetonitrile); gradient: 40%-60% B over 8 minutes] to afford the title compound (32.8 mg, 27% yield) as white solid; ¹H NMR (400 MHz, DMSO-d₆) δ=9.96 (br s, 1H), 9.17 (s, 1H), 8.17-8.09 (m, 1H), 7.77 (dd, J=6.0, 9.2 Hz, 1H), 7.59-7.53 (m, 1H), 7.41-7.30 (m, 2H), 7.07 (s, 1H), 7.01 (d, J=2.0 Hz, 1H), 5.37-5.18 (m, 1H), 4.22-4.13 (m, 5H), 4.10-4.04 (m, 1H), 3.90-3.80 (m, 4H), 3.13-2.99 (m, 3H), 2.77 (s, 1H), 2.43-2.34 (m, 1H), 2.20-2.06 (m, 2H), 2.06-1.96 (m, 2H), 1.88-1.74 (m, 3H), 0.76-0.68 (m, 3H); LCMS (ESI, M+1): m/z=673.3.

### Example 151

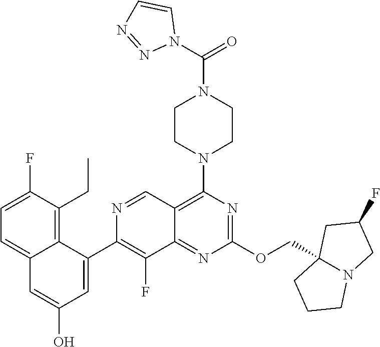 [SMILES: CCc1c(F)ccc2cc(O)cc(-c3ncc4c(N5CCN(C(=O)n6ccnn6)CC5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(1H-1,2,3-triazol-1-yl)methanone

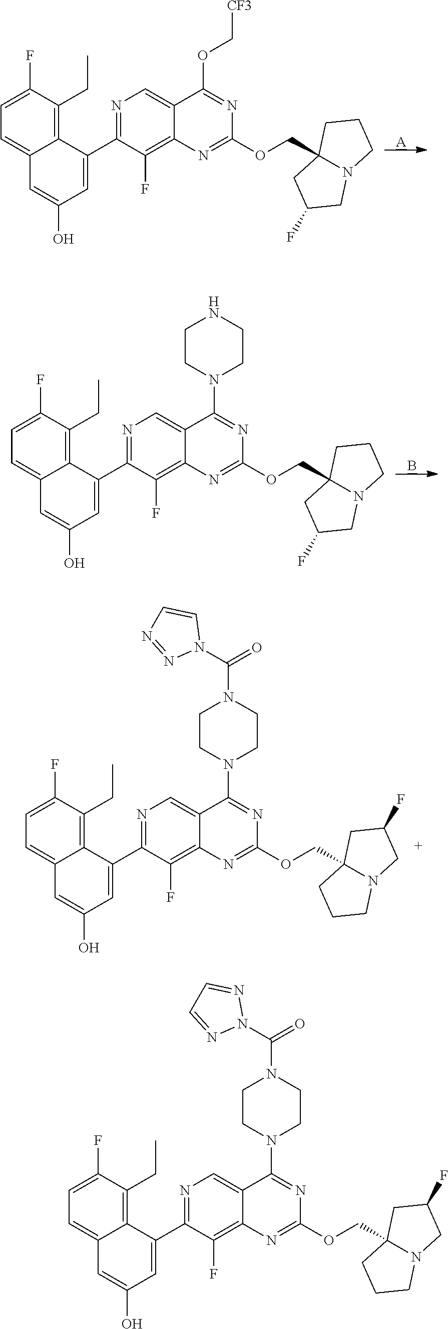

Step A. 5-ethyl-6-fluoro-4-(8-fluoro-2-(((2R,7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol: To a solution of piperazine (726 mg, 5.0 equiv) in DMF (10 mL) was added 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (1.00 g, 1.0 equiv) at 25 °C. The reaction was stirred at 25 °C for 2 hours. The mixture was purified with reversed phase flash [water/acetonitrile=1/1] to afford the title compound (914 mg, 90% yield) as yellow solid; LCMS (ESI, M+1): m/z=579.4.

Step B. (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(1H-1,2,3-triazol-1-yl)methanone and (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(2H-1,2,3-triazol-2-yl)methanone: To a solution of 1H-1,2,3-triazole (140 mg, 1.0 equiv) and DIEA (1.05 mg, 4.0 equiv) in DCM (2.5 mL) was added bis(trichloromethyl) carbonate (620 mg, 2.0 eq) at 0 °C. The reaction was stirred at 0 °C for 1 hour. 5-ethyl-6-fluoro-4-(8-fluoro-2-(((2R,7aS)-2-fluorotetrahydro-1H-pyrrolizin-7a(5H)-yl)methoxy)-4-(piperazin-1-yl)pyrido[4,3-d]pyrimidin-7-yl)naphthalen-2-ol (641 mg, 0.9 equiv) and DIEA (392 mg, 1.5 equiv) in DCM (6 mL) were added dropwise into above mixture at 0 °C. The reaction was stirred at 20 °C for 14 hours. The mixture was quenched with saturated NaHCO₃ aqueous (20 mL) and extracted with DCM (4×15 mL). The combined organic layers were washed with brine (20 mL), dried over anhydrous sodium sulfate, concentrated and purified with reversed phase flash [water (0.1% formic acid)/acetonitrile] and SFC [Column: Chiralpak IK-3×50×4.6 mm I.D., 3 μm; Mobile phase: Phase A for CO₂, and Phase B for IPA+acetonitrile (0.05% DEA); Gradient elution: 50% IPA+acetonitrile (0.05% DEA) in CO₂ Flow rate: 3 mL/minute; Detector: PDA; Column Temp: 35 °C; Back Pressure: 100Bar] to afford two peaks.

Peak 1:(4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(1H-1,2,3-triazol-1-yl)methanone (100 mg) was further purified with prep-HPLC [column: Waters xbridge 150×25 mm×10 μm; mobile phase: water (NH₄HCO₃)-acetonitrile; gradient: 38%-58% B over 8 minutes] to afford the title compound (28.7 mg, 3% yield) as white solid; ¹H NMR (400 MHz, DMSO-d₆) δ=9.96 (s, 1H), 9.19 (s, 1H), 8.61 (d, J=1.2 Hz, 1H), 7.95 (d, J=1.2 Hz, 1H), 7.77 (dd, J=6.0, 9.2 Hz, 1H), 7.40-7.30 (m, 2H), 7.02 (d, J=2.0 Hz, 1H), 5.42-5.18 (m, 1H), 4.41-4.08 (m, 6H), 4.01-3.90 (m, 4H), 3.24-2.99 (m, 3H), 2.93-2.77 (m, 1H), 2.42-2.31 (m, 1H), 2.24-1.97 (m, 4H), 1.93-1.72 (m, 3H), 0.73 (t, J=7.2 Hz, 3H); LCMS (ESI, M+1): m/z=674.4; SFC [Column: Chiralpak IK-3 50×4.6 mm I.D., 3 μm; Mobile phase: Phase A for CO₂, and Phase B for IPA+CAN (0.05% DEA); Gradient elution: 50% IPA+ACN (0.05% DEA) in CO₂; Flow rate: 3 mL/min; Detector: PDA; Column Temp: 35 °C; Back Pressure: 100Bar]. RT=1.255 minutes.

### Example 152

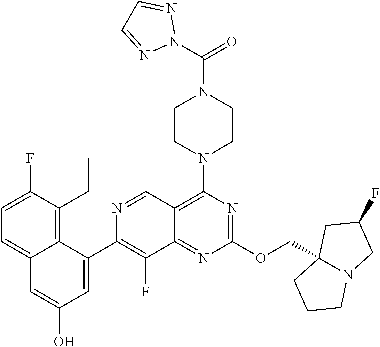 [SMILES: CCc1c(F)ccc2cc(O)cc(-c3ncc4c(N5CCN(C(=O)n6nccn6)CC5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(2H-1,2,3-triazol-2-yl)methanone

Peak 2: (4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperazin-1-yl)(2H-1,2,3-triazol-2-yl)methanone (130 mg) was purified with prep-HPLC [column: Waters Xbridge 150×25 mm×5 μm; mobile phase: water (NH₄HCO₃)-acetonitrile; gradient: 38%-58% B over 9 minutes] to afford the title compound (42.1 mg, 4.4% yield) as white solid; ¹H NMR (400 MHz, DMSO-d₆) δ=9.94 (s, 1H), 9.18 (s, 1H), 8.18 (s, 2H), 7.77 (dd, J=6.0, 9.2 Hz, 1H), 7.39-7.30 (m, 2H), 7.02 (d, J=2.0 Hz, 1H), 5.38-5.17 (m, 1H), 4.30-3.83 (m, 10H), 3.15-3.03 (m, 2H), 3.01 (s, 1H), 2.88-2.77 (m, 1H), 2.39-2.30 (m, 1H), 2.16-1.97 (m, 4H), 1.90-1.70 (m, 3H), 0.72 (t, J=7.6 Hz, 3H); LCMS (ESI, M+1): m/z=674.3; SFC [Column: Chiralpak IK-3 50×4.6 mm I.D., 3 μm; Mobile phase: Phase A for CO₂, and Phase B for IPA+CAN (0.05% DEA); Gradient elution: 50% IPA+ACN (0.05% DEA) in CO₂; Flow rate: 3 mL/min; Detector: PDA; Column Temp: 35C; Back Pressure: 100Bar]. RT=1.885 minutes

### Example 153

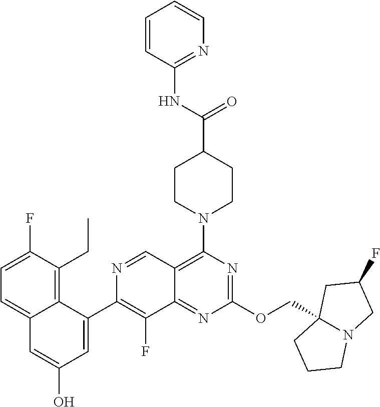 [SMILES: CCc1c(F)ccc2cc(O)cc(-c3ncc4c(N5CCC(C(=O)Nc6ccccn6)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-(pyridin-2-yl)piperidine-4-carboxamide

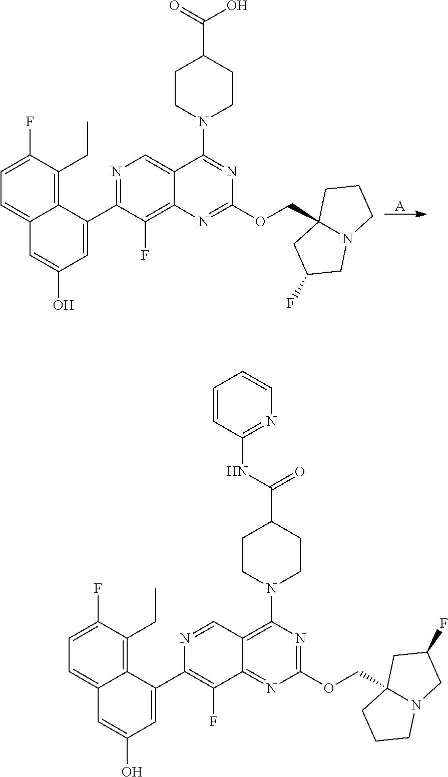

Step A. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-(pyridin-2-yl)piperidine-4-carboxamide: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 equiv) in DMF (2 mL) were added HATU (122 mg, 2.0 equiv) and DIEA (125 mg, 6.0 equiv). The reaction was stirred at 60 °C for 1 hour. To the above mixture was added pyridin-2-amine (75.7 mg, 5.0 equiv). The reaction was stirred at 60 °C for 11 hours. The mixture was filtered and purified by Prep-HPLC [Waters Xbridge C18 150×25 mm×5 μm; A: water (NH₄HCO₃), B: ACN; B %: 42%-72% B over 9 min] followed by Prep-HPLC [Welch Xtimate C18 150×25 mm×5 μm; A: water(FA), B: ACN; B %: 13%-43% B over 10 min] to afford the title compound (4.04 mg, 3.5% yield, 0.18 HCOOH) as white solid; ¹H NMR (400 MHz, CHLOROFORM-d) δ=8.95 (d, J=5.6 Hz, 1H), 8.33-8.24 (m, 2H), 8.20 (br d, J=8.0 Hz, 1H), 7.73 (t, J=12 Hz, 1H), 7.52 (dd, J=6.4, 8.8 Hz, 1H), 7.22-7.13 (m, 2H), 7.12-7.06 (m, 1H), 7.06-6.95 (m, 1H), 5.44-5.19 (m, 1H), 4.67-4.49 (m, 2H), 4.38-4.24 (m, 2H), 3.49-3.16 (m, 5H), 3.07-2.93 (m, 1H), 2.68-2.57 (m, 1H), 2.45 (br d, J=7.2 Hz, 2H), 2.36-2.05 (m, 10H), 0.80 (t, J=7.4 Hz, 3H); LCMS (ESI, M+1): m/z=698.3.

### Example 154

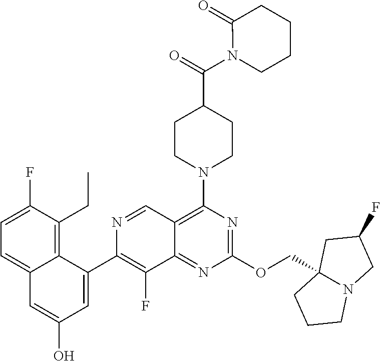 [SMILES: CCc1c(F)ccc2cc(O)cc(-c3ncc4c(N5CCC(C(=O)N6CCCCC6=O)CC5)nc(OC[C@@]56CCCN5C[C@H](F)C6)nc4c3F)c12]

### 1-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carbonyl)piperidin-2-one

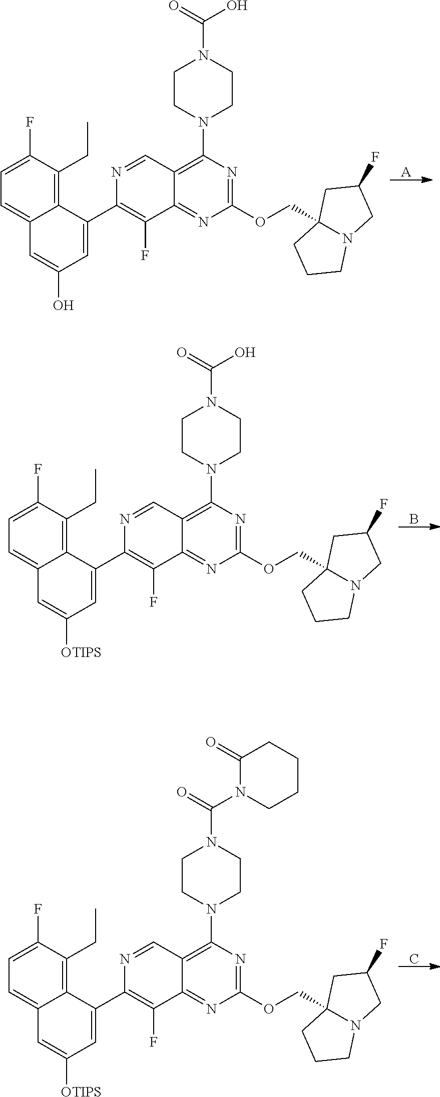

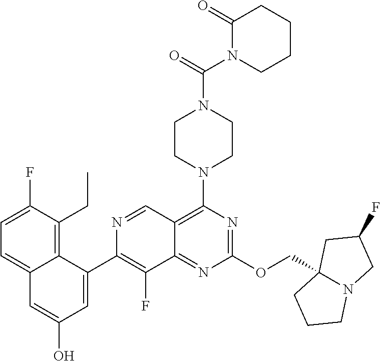 [SMILES: CCc1c(F)ccc2cc(O)cc(-c3ncc4c(N5CCN(C(=O)N6CCCCC6=O)CC5)nc(OC[C@@]56CCCN5C[C@H](F)C6)nc4c3F)c12]

Step A. 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (500 mg, 1.0 equiv) and imidazole (821 mg, 15 equiv) in DMF (8.00 mL) were added TIPSCl (1.55 g, 10 equiv) and DMAP (49.1 mg, 0.5 equiv). The reaction was stirred at 40 °C for 16 hours. The mixture was quenched with H₂O (50 mL) and extracted with heptane (3×20 mL). The combined organic layers were dried over Na₂SO₄, filtered and concentrated in vacuum to give crude triisopropylsilyl 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate. The crude was dissolved in MeOH (3.00 mL) and THF (3.00 mL) and added aq. K₂CO₃ (2.0 equiv, 1 M). The reaction was stirred at 25 °C for 2 hours. The pH was adjusted to 2-3 with 1 M aq. HCl (20 mL) at 0 °C. The aqueous layer was extracted with PE (3×10 mL). The combined organic layers were dried over Na₂SO₄, concentrated and purified with reversed phase flash chromatography [C18, 0.1% formic acid condition] to afford the title compound (1.60 g, 81% yield) as yellow oil; ¹H NMR (400 MHz, DMSO-d6) δ=10.92-10.73 (m, 1H), 9.14 (s, 1H), 7.91 (dd, J=6.0, 9.2 Hz, 1H), 7.51 (d, J=2.8 Hz, 1H), 7.41 (t, J=9.2 Hz, 1H), 7.07 (d, J=2.4 Hz, 1H), 5.68-5.47 (m, 1H), 4.65-4.43 (m, 4H), 3.90-3.81 (m, 2H), 2.81-2.69 (m, 2H), 2.41-2.29 (m, 3H), 2.23-2.02 (m, 7H), 1.87-1.74 (m, 2H), 1.36-1.26 (m, 3H), 1.09 (s, 9H), 1.07 (s, 9H), 0.97 (s, 3H), 0.73 (br t, J=7.6 Hz, 3H); LCMS (ESI, M+1): m/z=778.4.

Step B. 1-(1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carbonyl)piperidin-2-one: To a solution of 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (120 mg, 1.0 equiv) and piperidin-2-one (45.9 mg, 3.0 equiv) in THF (2.00 mL) were added DIEA (99.7 mg, 5.0 equiv) and CMPI (118 mg, 3.0 equiv). The reaction was stirred at 25 °C for 1 hour. The mixture was diluted with water (30 mL) and extracted with ethyl acetate (2×15 mL). The combined organic layers were washed with brine, dried over anhydrous sodium sulfate, concentrated to afford the title compound (100 mg, crude) as yellow solid; LCMS (ESI, M+1): m/z=859.5.

Step C. 1-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carbonyl)piperidin-2-one: To a solution of 1-(1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carbonyl)piperidin-2-one (100 mg, 1.0 equiv) in DMF (3.00 mL) was added CsF (177 mg, 10 equiv). The reaction was stirred at 25 °C for 0.5 hours. The mixture was filtered and purified with prep-HPLC [column: Waters Xbridge 150×25 mm×5 μm; A: water (NH₄HCO₃), B: ACN; B %: 48%-78% over 9 min] to afford the title compound (12.6 mg, 14% yield) as white solid; ¹H NMR (400 MHz, DMSO-d6) δ=9.93 (s, 1H), 9.07 (s, 1H), 7.76 (dd, J=6.0, 9.0 Hz, 1H), 7.39-7.30 (m, 2H), 7.02 (d, J=2.3 Hz, 1H), 5.38-5.18 (m, 1H), 4.53 (br d, J=9.2 Hz, 2H), 4.19-4.03 (m, 2H), 3.79-3.66 (m, 1H), 3.62 (br t, J=5.6 Hz, 2H), 3.53-3.40 (m, 2H), 3.13-3.00 (m, 3H), 2.86-2.79 (m, 1H), 2.41-2.29 (m, 1H), 2.19-2.09 (m, 2H), 2.09-1.93 (m, 5H), 1.89-1.68 (m, 10H), 0.72 (t, J=7.2 Hz, 3H); LCMS (ESI, M+1): m/z=703.3.

### Example 155

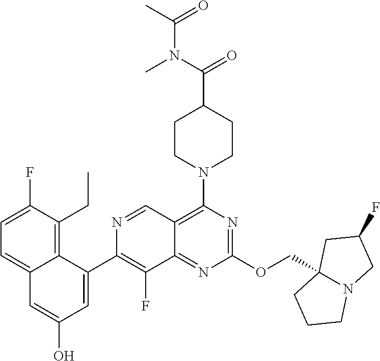 [SMILES: CCc1c(F)ccc2cc(O)cc(-c3ncc4c(N5CCC(C(=O)N(C)C(C)=O)CC5)nc(OC[C@@]56CCCN5C[C@H](F)C6)nc4c3F)c12]

### N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide

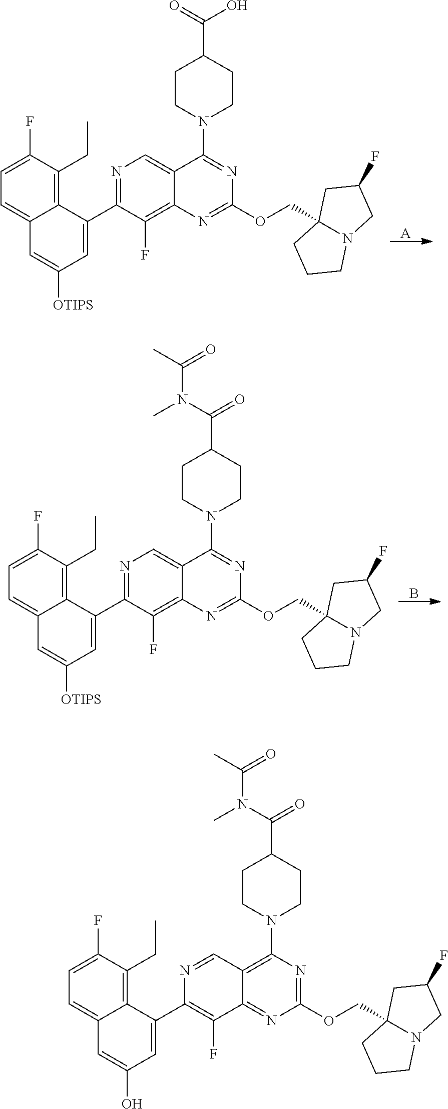

Step A. N-acetyl-1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide: To a solution of 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 equiv) and N-methylacetamide (28.2 mg, 3.0 equiv) in THF (1 mL) were added CMPI (164 mg, 5.0 equiv) and DIEA (83.1 mg, 5.0 equiv). The reaction was stirred at 30 °C for 3 hours. The mixture was diluted with water (2 mL) and extracted with ethyl acetate (3 mL). The organic layer was dried over sodium sulfate, concentrated and purified by reverse-phase flash chromatography [C18, 0.1% NH₃·H₂O condition] to afford the title compound (90.0 mg, 66% yield) as yellow solid. LCMS (ESI, M+1): m/z=833.5

Step B: N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide: To a solution of N-acetyl-1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide (80.0 mg, 1.0 equiv) in DMF (1 mL) was added CsF (146 mg, 10 equiv). The reaction was stirred at 30 °C for 1 hour. The mixture was filtered and purified by prep-HPLC [Waters Xbridge 150×25 mm×5 μm; A: water (NH₄HCO₃), B: ACN, B %: 44%-74% over 9 min] to afford the title compound (90.0 mg, 66% yield) as yellow solid. ¹H NMR (400 MHz, CHLOROFORM-d) δ=8.97 (s, 1H), 7.64-7.51 (m, 1H), 7.24-7.20 (m, 2H), 7.08-7.02 (m, 1H), 5.38-5.20 (m, 2H), 4.66-4.58 (m, 2H), 4.33-4.22 (m, 2H), 3.65 (br d, J=3.6 Hz, 1H), 3.48-3.37 (m, 2H), 3.31 (br dd, J=3.2, 7.4 Hz, 1H), 3.27 (s, 3H), 3.24-3.16 (m, 1H), 3.04-2.95 (m, 1H), 2.52-2.44 (m, 1H), 2.41 (s, 3H), 2.31 (br s, 1H), 2.23 (br t, J=7.2 Hz, 2H), 2.19-2.13 (m, 2H), 2.09-2.04 (m, 2H), 2.03-1.96 (m, 4H), 1.94 (br s, 1H), 0.84-0.81 (m, 3H). LCMS (ESI, M+1): m/z=677.5

### Example 156

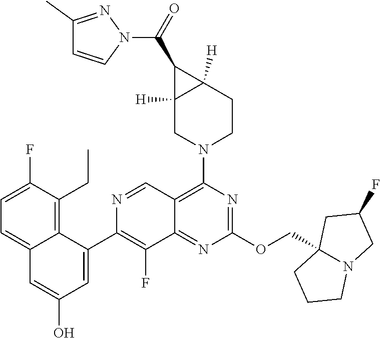 [SMILES: CCc1c(F)ccc2cc(O)cc(-c3ncc4c(N5CC[C@H]6[C@@H](C5)[C@H]6C(=O)n5ccc(C)n5)nc(OC[C@@]56CCCN5C[C@H](F)C6)nc4c3F)c12]

### ((1R,6S,7S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptan-7-yl)(3-methyl-1H-pyrazol-1-yl)methanone

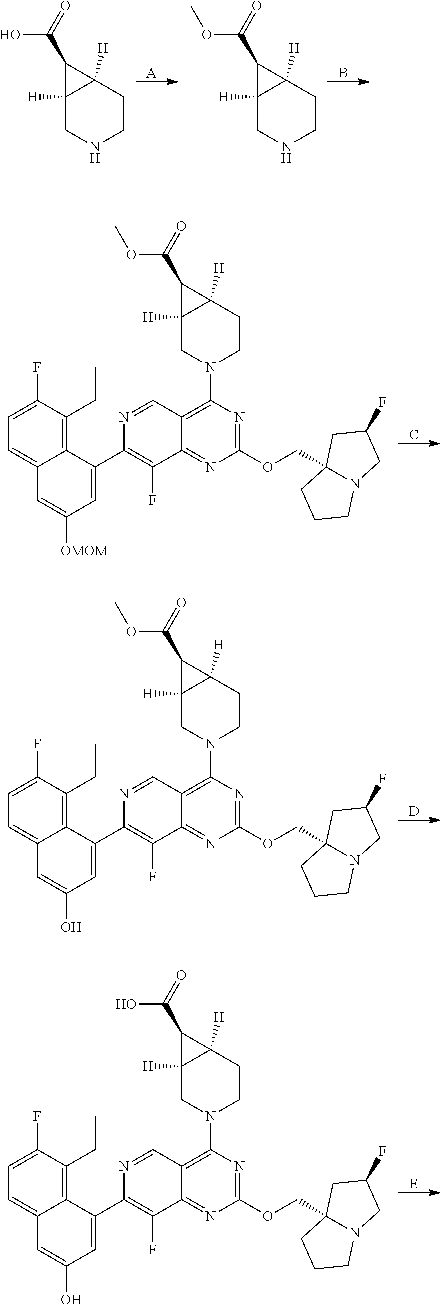

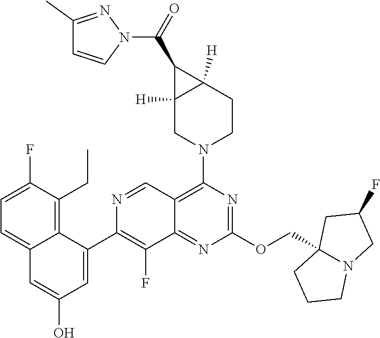 [SMILES: CCc1c(F)ccc2cc(O)cc(-c3ncc4c(N5CC[C@H]6[C@@H](C5)[C@H]6C(=O)n5ccc(C)n5)nc(OC[C@@]56CCCN5C[C@H](F)C6)nc4c3F)c12]

Step A. methyl (1R,6S,7S)-3-azabicyclo[4.1.0]heptane-7-carboxylate: A solution of (1R,6S,7S)-3-azabicyclo[4.1.0]heptane-7-carboxylic acid (500 mg, 1.0 equiv, HCl) in HCl·MeOH (2 M, 10.0 mL, 7.1 equiv) was stirred at 25 °C for 16 hours. The mixture was concentrated to afford the title compound (600 mg, crude, HCl) as yellow oil.

Step B. methyl (1R,6S,7S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 equiv) in DMF (5 mL) were added DIEA (699 mg, 6.0 equiv) and PyBOP (704 mg, 1.5 equiv). The reaction was stirred at 25 °C for 0.5 hours. methyl (1R,6S,7S)-3-azabicyclo[4.1.0]heptane-7-carboxylate (346 mg, 2.0 equiv, HCl) was added into the mixture. The reaction was stirred at 25 °C for 1.5 hours. The mixture was filtered and purified by prep-HPLC [Waters Xbridge Prep OBD C18 150×40 mm×10 μm; A: water (NH₄HCO₃), B %: ACN, B %: 52%-82% over 20 min] and prep-chiral SFC [DAICEL CHIRALPAK AD (250 mm×30 mm, 10 μm); mobile phase: [CO₂-ACN/i-PrOH (0.1% NH₃H₂O)]; B %: 45%, isocratic elution mode] to afford the title compound (200 mg, 32% yield) as yellow oil.

Step C. methyl (1R,6S,7S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate: To a solution of methyl (1R,6S,7S)-3-(7-(8-ethyl-7-fluoro-3-(methoxymethoxy)naphthalen-1-yl)-8-fluoro-2-yl)-3-azabicyclo[4.1.0]heptane-7-carboxylate (70.0 mg, 1.0 equiv) in MeOH (0.5 mL) was added HCl·MeOH (2 M, 2.00 mL, 40 equiv). The reaction was stirred at 25 °C for 1 hour. The mixture was concentrated, dissolved with MeOH (5 mL), adjusted to pH=7 using NaHCO₃ solid and concentrated to afford the title compound (110 mg, crude) as white solid, LCMS (ESI, M+1): m/z=648.3.

Step D. (1R,6S,7S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid: To a solution of methyl (1R,6S,7S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate (110 mg, 1.0 equiv) in MeOH (1 mL) was added LiOH·H₂O (2 M, 1.00 mL, 12 equiv). The reaction was stirred at 25 °C for 1 hour. The mixture was filtered and purified by prep-HPLC [Waters Xbridge 150×25 mm×5 μm; A: water (NH₄HCO₃), B %: ACN, B %: 15%-45% over 15 min] to afford the title compound (10.0 mg, 9.3% yield) as white solid; LCMS (ESI, M+1): m/z=634.4.

Step E. ((1R,6S,7S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptan-7-yl)(3-methyl-1H-pyrazol-1-yl)methanone: To a solution of (1R,6S,7S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid (10.0 mg, 1.0 equiv) and 3-methyl-1H-pyrazole (3.89 mg, 3.0 equiv) in DMF (0.5 mL) were added HATU (12.0 mg, 2.0 equiv) and DIEA (10.2 mg, 5.0 equiv). The reaction was stirred at 25 °C for 1 hour. The reaction mixture was filtered and purified by prep-HPLC [Waters Xbridge 150×25 mm×5 μm; A: water (NH₄HCO₃), B %: ACN, B %: 50%-80% over 9 min] to afford the title compound (4.59 mg, 41% yield) as off-white solid; ¹H NMR (400 MHz, CHLOROFORM-d) δ=8.94 (d, J=3.2 Hz, 1H), 8.23-8.05 (m, 1H), 7.67-7.47 (m, 1H), 7.25 (br d, J=2.4 Hz, 2H), 7.02-6.91 (m, 1H), 6.40-6.17 (m, 1H), 5.57-5.15 (m, 1H), 4.43-4.26 (m, 3H), 3.96-3.82 (m, 1H), 3.69-3.57 (m, 1H), 3.51 (br s, 2H), 3.29-3.12 (m, 3H), 3.07-2.91 (m, 2H), 2.89-2.69 (m, 2H), 2.55-2.38 (m, 2H), 2.31 (d, J=5.2 Hz, 3H), 2.23-2.09 (m, 5H), 1.99-1.89 (m, 2H), 0.81 (td, J=7.2, 12.4 Hz, 3H); LCMS (ESI, M+1): m/z=698.3.

### Example 157

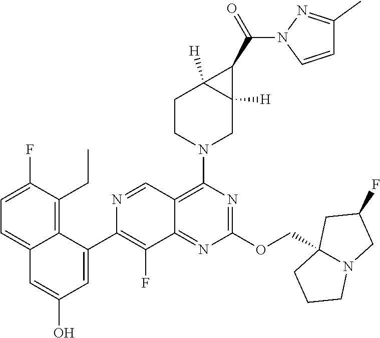 [SMILES: CCc1c(F)ccc2cc(O)cc(-c3ncc4c(N5CC[C@@H]6[C@H](C5)[C@@H]6C(=O)n5ccc(C)n5)nc(OC[C@@]56CCCN5C[C@H](F)C6)nc4c3F)c12]

### ((1S,6R,7R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptan-7-yl)(3-methyl-1H-pyrazol-1-yl)methanone

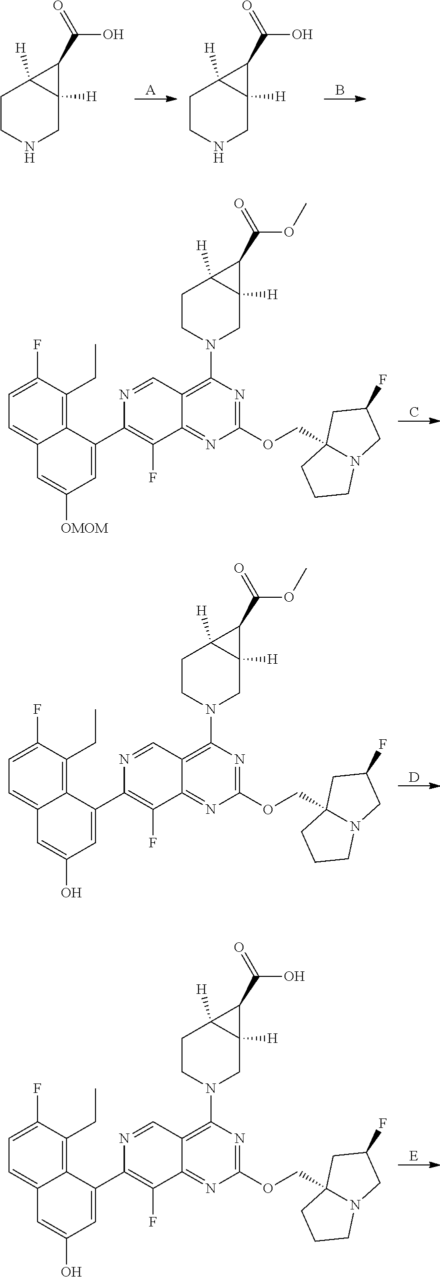

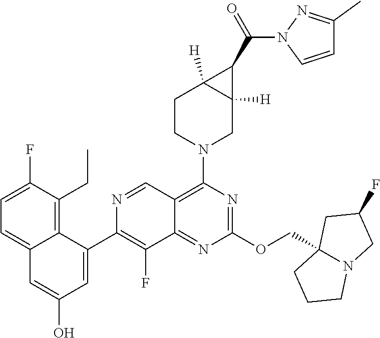 [SMILES: CCc1c(F)ccc2cc(O)cc(-c3ncc4c(N5CC[C@@H]6[C@H](C5)[C@@H]6C(=O)n5ccc(C)n5)nc(OC[C@@]56CCCN5C[C@H](F)C6)nc4c3F)c12]

Step A. methyl (1S,6R,7R)-3-azabicyclo[4.1.0]heptane-7-carboxylate: A solution of (1S,6R,7R)-3-azabicyclo[4.1.0]heptane-7-carboxylic acid (500 mg, 1.0 equiv, HCl) in HCl·MeOH (2 M, 10.0 mL, 7.1 equiv) was stirred at 25 °C for 16 hours. The mixture was concentrated to afford the title compound (600 mg, crude, HCl) as yellow oil.

Step B. methyl (1S,6R,7R)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 equiv) in DMF (5 mL) were added DIEA (699 mg, 6.0 equiv) and PyBOP (704 mg, 1.5 equiv). The reaction was stirred at 25 °C for 0.5 hours. methyl (1S,6R,7R)-3-azabicyclo[4.1.0]heptane-7-carboxylate (346 mg, 2.0 equiv, HCl) was added into the mixture. The reaction was stirred at 25 °C for 1.5 hours. The mixture was filtered and purified by prep-HPLC [Waters Xbridge Prep OBD C18 150×40 mm×10 μm; A: water (NH₄HCO₃), B %: ACN, B %: 52%-82% over 20 min] and prep-chiral SFC [DAICEL CHIRALPAK AD (250 mm×30 mm, 10 μm); mobile phase: [CO₂-ACN/i-PrOH (0.1% NH₃H₂O)]; B %: 45%, isocratic elution mode] to afford the title compound (200 mg, 32% yield) as yellow oil.

Step C. methyl (1S,6R,7R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate: To a solution of methyl (1S,6R,7R)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate (70.0 mg, 1.0 equiv) in MeOH (0.5 mL) was added HCl·MeOH (2 M, 2.00 mL, 40 equiv). The reaction was stirred at 25 °C for 2 hours. The mixture was concentrated, dissolved with MeOH (5 mL), adjusted to pH=7 using NaHCO₃solid and concentrated to afford the title compound (70.0 mg, crude) as white solid; LCMS (ESI, M+1): m/z=648.3.

Step D. (1S,6R,7R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid: To a solution of methyl (1S,6R,7R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate (70.0 mg, 1.0 equiv) in MeOH (0.5 mL) was added LiOH·H₂O (2 M, 0.5 mL, 11 equiv). The reaction was stirred at 25 °C for 1 hour. The mixture was filtered and purified by reversed-phase flash (0.1% FA condition) to afford the title compound (30.0 mg, 47% yield, HCOOH) as white solid; LCMS (ESI, M+1): m/z=634.3.

Step E. ((1S,6R,7R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptan-7-yl)(3-methyl-1H-pyrazol-1-yl)methanone: To a solution of (1S,6R,7R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid (30.0 mg, 1.0 equiv, HCOOH) and 3-methyl-1H-pyrazole (10.9 mg, 3.0 equiv) in DMF (0.5 mL) were added HATU (33.6 mg, 2.0 equiv) and DIEA (28.5 mg, 5.0 equiv). The reaction was stirred at 25 °C for 2 hours. The reaction mixture was filtered and purified by prep-HPLC [Waters Xbridge 150×25 mm×5 μm; A: water (NH₄HCO₃), B %: ACN, B %: 50%-80% over 9 min] to afford the title compound (8.59 mg, 25% yield) as yellow solid; ¹H NMR (400 MHz, CHLOROFORM-d) δ=1.55 (br t, J=7.44 Hz, 3H), 1.99-2.04 (m, 2H), 2.10-2.20 (m, 4H), 2.21-2.26 (m, 1H), 2.31 (d, J=7.44 Hz, 4H), 2.33-2.39 (m, 1H), 2.41 (br s, 2H), 3.01-3.13 (m, 3H), 3.15-3.39 (m, 3H), 3.46-3.60 (m, 2H), 3.78-3.89 (m, 1H), 4.27-4.41 (m, 2H), 4.46-4.66 (m, 1H), 5.22-5.47 (m, 1H), 6.27 (dd, J=4.24, 2.88 Hz, 1H), 6.91-7.04 (m, 1H), 7.13-7.24 (m, 2H), 7.41-7.61 (m, 1H), 8.14 (t, J=3.16 Hz, 1H), 8.93 (d, J=7.76 Hz, 1H), 10.76-11.51 (m, 1H); LCMS (ESI, M+1): m/z=698.3.

### Example 158

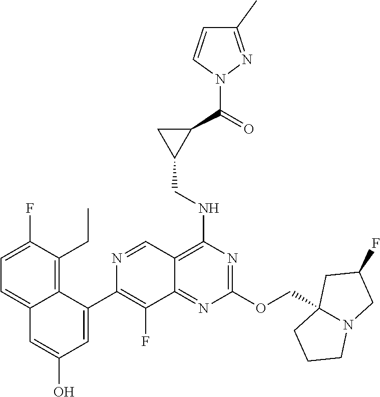 [SMILES: CCc1c(F)ccc2cc(O)cc(-c3ncc4c(NC[C@@H]5C[C@H]5C(=O)n5ccc(C)n5)nc(OC[C@@]56CCCN5C[C@H](F)C6)nc4c3F)c12]

### ((1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropyl)(3-methyl-1H-pyrazol-1-yl)methanone

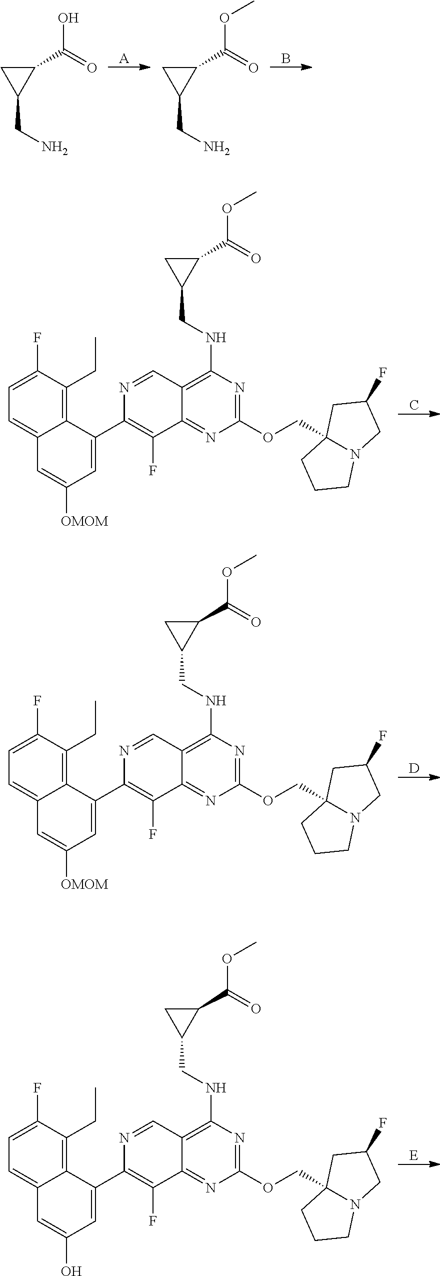

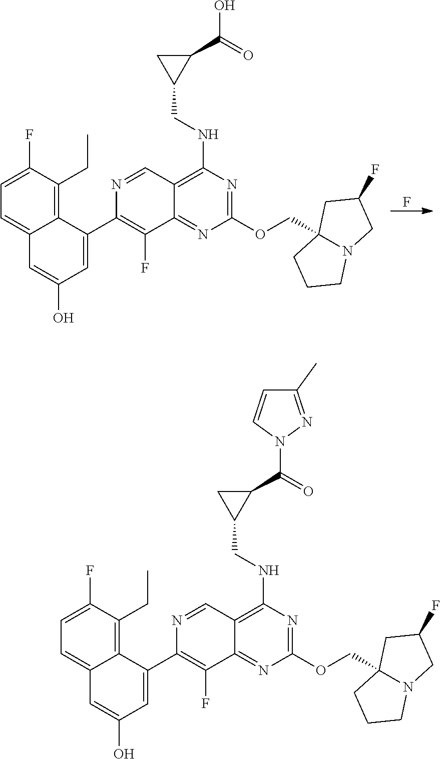

Step A. methyl (1S,2S)-2-(aminomethyl)cyclopropane-1-carboxylate: A solution of (1S,2S)-2-(aminomethyl)cyclopropane-1-carboxylic acid (500 mg, 1.0 equiv) in HCl·MeOH (10 mL, 2 M) was stirred at 25 °C for 12 hours. The mixture was concentrated under reduced pressure to remove solvent to afford the title compound (710 mg, crude, HCl) as white solid; ¹H NMR (400 MHz, METHANOL-d₄) δ=3.76-3.64 (m, 3H), 3.02-2.83 (m, 2H), 1.79 (td, J=4.4, 8.4 Hz, 1H), 1.74-1.63 (m, 1H), 1.30-1.21 (m, 1H), 1.04 (ddd, J=4.4, 6.0, 8.4 Hz, 1H).

Step B. methyl (1S,2S)-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (1.00 g, 1.0 equiv) in DMF (10 mL) were added PyBOP (1.41 g, 1.5 equiv), methyl (1S,2S)-2-(aminomethyl)cyclopropane-1-carboxylate (448 mg, 1.5 equiv) and DIEA (1.17 g, 5.0 equiv). The reaction was stirred at 25 °C for 1 hour. The mixture was diluted with water (60 mL) and extracted with ethyl acetate (40 mL×2). The combined organic layers were washed with brine (80 mL), dried over anhydrous sodium sulfate, concentrated and purified with prep-HPLC [neutral condition; column: Waters Xbridge Prep OBD C18 150×40 mm×10 μm; mobile phase: water (NH₄HCO₃)-ACN; gradient: 46%-76% B over 20 min] to afford the title compound (900 mg, 74% yield) as white solid; LCMS (ESI, M+1): m/z=666.3.

Step C. methyl (1R,2R)-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate: Methyl (1R,2R)-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate (0.90 g) was purified with SFC (column: DAICEL CHIRALCEL OD (250 mm×30 mm, 10 μm); mobile phase: [CO₂-MeOH (0.1% NH₃H₂O)]; B %: 45%, isocratic elution mode) to afford two peaks.

Peak 1: methyl (1R,2R)-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate (395 mg, 43% yield) as brown solid; SFC: Column: Chiralcel OD-3 50×4.6 mm I.D., 3umMobile phase: Phase A for CO₂, and Phase B for MeOH (0.05% DEA); Gradient elution: 40% MeOH (0.05% DEA) in CO₂, Flow rate: 3 mL/min; Detector: PDA; Column Temp: 35C; Back Pressure: 100Bar, t_{R}: 0.777 min; LCMS (ESI, M+1): m/z=666.3.

Peak 2: methyl (1S,2S)-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate (401 mg, 44% yield) as brown solid; SFC: Column: Chiralcel OD-3 50×4.6 mm I.D., 3umMobile phase: Phase A for CO₂, and Phase B for MeOH (0.05% DEA); Gradient elution: 40% MeOH (0.05% DEA) in CO₂, Flow rate: 3 mL/min; Detector: PDA; Column Temp: 35C; Back Pressure: 100Bar, t_{R}: 1.248 min; LCMS (ESI, M+1): m/z=666.3.

Step D. methyl (1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate: To a solution of methyl (1R,2R)-2-(((7-(8-ethyl-7-fluoro-3-(methoxymethoxy)naphthalen-1-yl)-8-fluoro-2-yl)amino)methyl)cyclopropane-1-carboxylate (375 mg, 1.0 equiv) in MeCN (4 mL) was added HCl·dioxane (4 mL, 2M). The mixture was stirred at 20 °C for 0.5 hours. The mixture was concentrated under reduced pressure, diluted with NaHCO₃ (10 mL) and extracted with DCM (15 mL×2). The combined organic layers were washed with brine (30 mL), dried over anhydrous sodium sulfate and concentrated to afford the title compound (340 mg, crude) as brown solid; LCMS (ESI, M+1): m/z=622.4.

Step E. (1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylic acid: To a solution of methyl (1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate (310 mg, 1.0 equiv) in MeOH (3 mL) was added LiOH (1 mL, 2 M). The reaction was stirred at 25 °C for 2 hours. The mixture was purified with prep-HPLC [FA condition; column: Phenomenex luna C18 150×25 mm×10 μm; mobile phase: [water(FA)-ACN]; gradient: 8%-38% B over 10 min] to afford the title compound (230 mg, 74% yield) as white solid; LCMS (ESI, M+1): m/z=608.4.

Step F. ((1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropyl)(3-methyl-1H-pyrazol-1-yl)methanone: To a solution of (1R,2R)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylic acid (60.0 mg, 1.0 equiv) in DMF (1 mL) were added HATU (56.3 mg, 1.5 equiv), 3-methyl-1H-pyrazole (12.2 mg, 1.5 equiv) and DIEA (63.8 mg, 5.0 equiv). The reaction was stirred at 25 °C for 1 hour. The mixture was purified with prep-HPLC [column: Waters Xbridge Prep OBD C18 150×40 mm×10 μm; mobile phase: [water (NH₄HCO₃)-ACN]; gradient: 40%-70% B over 20 min] to afford the title compound (16.1 mg, 24% yield) as off-white solid; ¹H NMR (400 MHz, CHLOROFORM-d) δ=8.87 (s, 1H), 8.08 (dd, J=2.4, 10.8 Hz, 1H), 7.84-7.57 (m, 1H), 7.46-7.34 (m, 1H), 7.09 (dt, J=4.4, 9.2 Hz, 1H), 7.02-6.93 (m, 1H), 6.89-6.75 (m, 1H), 6.25 (d, J=2.0 Hz, 1H), 5.36-5.17 (m, 1H), 4.37-4.19 (m, 2H), 4.13-3.82 (m, 1H), 3.35-3.12 (m, 4H), 3.06-2.90 (m, 3H), 2.53-2.33 (m, 2H), 2.31 (d, J=12.0 Hz, 3H), 2.27-2.08 (m, 3H), 2.07-1.94 (m, 3H), 1.54-1.46 (m, 1H), 1.18-1.06 (m, 1H), 0.72 (br t, J=7.2 Hz, 3H); LCMS (ESI, M+1): m/z=672.4.

### Example 159

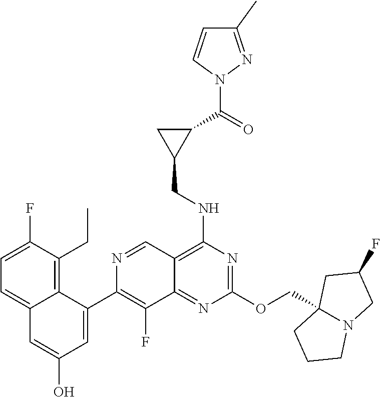 [SMILES: CCc1c(F)ccc2cc(O)cc(-c3ncc4c(NC[C@H]5C[C@@H]5C(=O)n5ccc(C)n5)nc(OC[C@@]56CCCN5C[C@H](F)C6)nc4c3F)c12]

### ((1S,2S)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropyl)(3-methyl-1H-pyrazol-1-yl)methanone

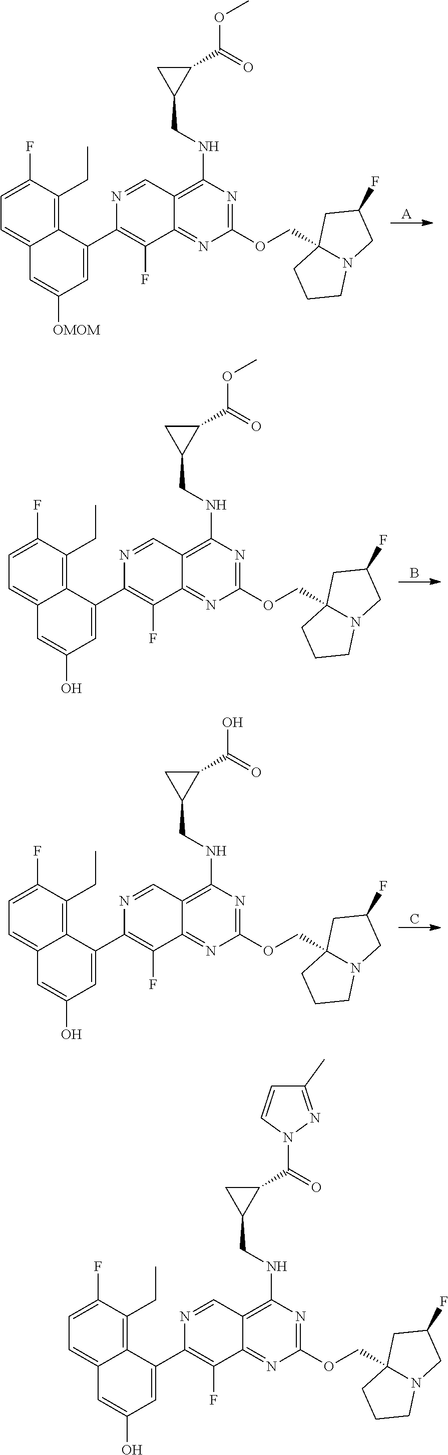

Step A. methyl (1S,2S)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate: To a solution of methyl (1S,2S)-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate (381 mg, 1.0 equiv) in MeCN (4 mL) was added HCl·dioxane (4 mL, 2M). The reaction was stirred at 20 °C for 0.5 hours. The mixture was concentrated under reduced pressure, diluted with NaHCO₃ (10 mL) and extracted with DCM (2×15 mL). The combined organic layers were washed with brine (30 mL), dried over anhydrous sodium sulfate and concentrated to afford the title compound (333 mg, crude) as brown solid; LCMS (ESI, M+1): m/z=622.4.

Step B. (1S,2S)-2-(((7-(8-ethyl-7-fluoro-3-hydroxynaphthalen-1-yl)-8-fluoro-2-yl)amino)methyl)cyclopropane-1-carboxylic acid: To a solution of methyl (1S,2S)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylate (310 mg, 1.0 equiv) in MeOH (3 mL) was added LiOH (1 mL, 2M). The reaction was stirred at 25 °C for 2 hours. The mixture was purified with prep-HPLC [FA condition; column: Phenomenex luna C18 150×25 mm×10 μm; mobile phase: [water (FA)-ACN]; gradient: 15%-45% B over 15 min] to afford the title compound (210 mg, 68% yield) as white solid; LCMS (ESI, M+1): m/z=608.3.

Step C. ((1S,2S)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropyl)(3-methyl-1H-pyrazol-1-yl)methanone: To a solution of (1S,2S)-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)cyclopropane-1-carboxylic acid (60.0 mg, 1.0 equiv) in DMF (1 mL) were added HATU (56.3 mg, 1.5 equiv), 3-methyl-1H-pyrazole (12.2 mg, 1.5 equiv) and DIEA (63.8 mg, 5.0 equiv). The reaction was stirred at 25 °C for 1 hour. The mixture was purified with prep-HPLC [column: Waters Xbridge Prep OBD C18 150×40 mm×10 μm; mobile phase: [water (NH₄HCO₃)-ACN]; gradient: 40%-70% B over 20 min] to afford the title compound (20.0 mg, 30% yield) as off-white solid; ¹H NMR (400 MHz, CHLOROFORM-d) δ=8.94-8.77 (m, 1H), 8.07 (dd, J=2.4, 14.8 Hz, 1H), 7.79-7.56 (m, 1H), 7.45-7.32 (m, 1H), 7.09 (dt, J=4.4, 9.2 Hz, 1H), 7.01-6.94 (m, 1H), 6.94-6.68 (m, 1H), 6.24 (d, J=2.5 Hz, 1H), 5.39-5.15 (m, 1H), 4.33-4.20 (m, 2H), 4.09-3.77 (m, 1H), 3.35-3.10 (m, 4H), 3.09-2.92 (m, 3H), 2.53-2.33 (m, 2H), 2.33-2.28 (m, 3H), 2.27-2.06 (m, 3H), 2.02-1.92 (m, 3H), 1.50 (tt, J=4.0, 8.8 Hz, 1H), 1.18-1.07 (m, 1H), 0.72 (br t, J=7.2 Hz, 3H); LCMS (ESI, M+1): m/z=672.4.

### Example 160

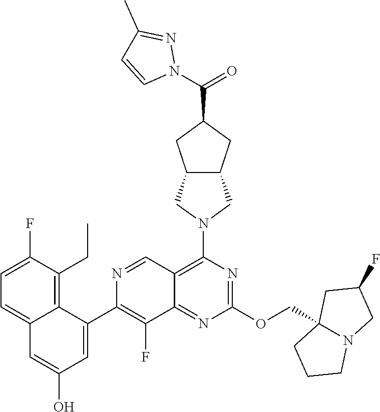 [SMILES: CCc1c(F)ccc2cc(O)cc(-c3ncc4c(N5C[C@H]6C[C@H](C(=O)n7ccc(C)n7)C[C@H]6C5)nc(OC[C@@]56CCCN5C[C@H](F)C6)nc4c3F)c12]

### ((3aR,5R,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone

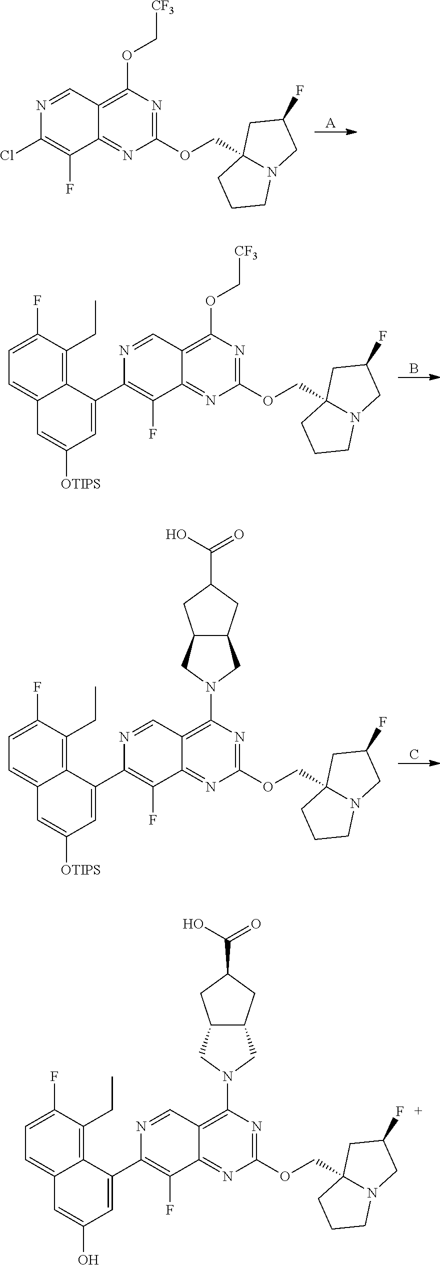

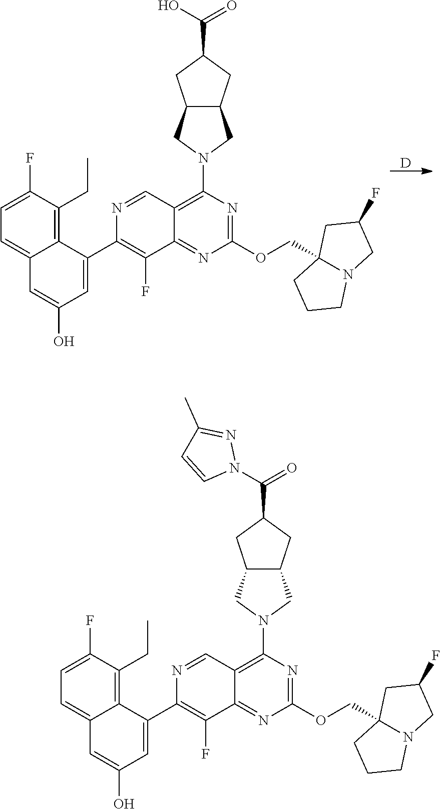

Step A. 7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (7.2 g, 1.0 equiv) and ((5-ethyl-6-fluoro-4-(4,4,5,5-tetramethyl-1,3,2-dioxaborolan-2-yl)naphthalen-2-yl)oxy)triisopropylsilane (11.6 g, 1.5 equiv) in methoxycyclopentane (71 mL) were added CataCXium A Pd G3 (1.2 g, 0.1 equiv) and Cs₂CO₃ (13.4 g, 2.5 equiv). The reaction was degassed and purged with nitrogen 3 times. The reaction was stirred at 100 °C for 3 hours. The mixture was diluted with water (100 mL) and extracted with ethyl acetate (2×60 mL). The combined organic layers were washed with brine (50 mL), dried over anhydrous sodium sulfate, concentrated and purified with reversed phase flash chromatography [C18, 0.1% formic acid condition] to afford the title compound (3.5 g, 26% yield) as yellow solid; LCMS (ESI, M+1): m/z=749.3.

Step B. (3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid: To a solution of 7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (500 mg, 1.0 equiv) and (3aR,6aS)-octahydrocyclopenta[c]pyrrole-5-carboxylic acid (124 mg, 1.2 equiv) in DMAC (5 mL) were added K₃PO₄ (425.18 mg, 3.0 equiv) and 4 Å molecular sieve (500 mg). The reaction was stirred at 60 °C for 1 hour. The mixture was filtered and purified by reversed phase flash chromatography [C18, 0.1% formic acid condition] to afford the title compound (410 mg, 44% yield) as yellow solid; LCMS (ESI, M+1): m/z=648.2.

Step C. (3aR,5R,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid: (3aR,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (410 mg, 1.0 equiv) was purified with SFC separation [column: DAICEL CHIRALPAK IC, 250×30 mm, 10 μm; A: CO₂, B: ACN/i-PrOH (30%/70%) (0.1% NH₃H₂O), B %: 50%-50% B over 3.7 min] and reversed phase flash chromatography [C18, 0.1% formic acid condition] to afford to afford two isomers.

Isomer 1:(3aR,5R,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (166 mg, 38% yield) as yellow solid; LCMS (ESI, M+1): m/z=648.3; SFC>99% ee, column: Chiralpak IC-3 50×4.6 mm I.D., 3 μm; mobile phase: 20% to 60% IPA+ACN (0.05% DEA) in CO₂, flow rate: 3 mL/min, detector: PDA, t_{R}: 1.668 min.

Isomer 2: (3aR,5S,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (32 mg, 6.8% yield) as yellow solid; LCMS (ESI, M+1): m/z=648.3; SFC=78% ee, column: Chiralpak IC-3 50×4.6 mm I.D., 3 μm; mobile phase: 20% to 60% IPA+ACN (0.05% DEA) in CO₂, flow rate: 3 mL/min, detector: PDA, t_{R}: 1.974 min.

Step D. ((3aR,5R,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrol-5-yl)(3-methyl-1H-pyrazol-1-yl)methanone: To a solution of (3aR,5R,6aS)-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (80 mg, 1.0 equiv) and 3-methyl-1H-pyrazole (50.7 mg, 5 equiv) in DMF (1 mL) were added HATU (93.9 mg, 2.0 equiv) and DIEA (79.8 mg, 5.0 equiv). The reaction was stirred at 25 °C for 1 hour. The mixture was filtered and purified by by prep-HPLC [Waters Xbridge 150×25 mm×5 μm; A: water (NH₄HCO₃), B: ACN, B %: 52%-82% over 9 min]. The desired fractions were diluted with water (50 mL) and extracted with dichloromethane (2×30 mL). The organic layers were dried over anhydrous sodium sulfate and concentrated to afford the title compound (10.1 mg, 11% yield) as off-white solid; ¹H NMR (400 MHz, CHLOROFORM-d) 1H NMR (400 MHz, CHLOROFORM-d) δ=9.13 (d, J=17.2 Hz, 1H), 8.16 (d, J=2.0 Hz, 1H), 7.47 (br t, J=5.6 Hz, 1H), 7.21-7.02 (m, 2H), 7.01-6.83 (m, 1H), 6.27 (d, J=2.4 Hz, 1H), 5.42-5.15 (m, 1H), 4.40-4.29 (m, 1H), 4.28-4.23 (m, 2H), 4.22-4.16 (m, 1H), 3.90-3.74 (m, 2H), 3.37-3.13 (m, 3H), 2.99 (br d, J=5.2 Hz, 3H), 2.53-2.42 (m, 1H), 2.37-2.27 (m, 6H), 2.26-2.07 (m, 5H), 2.04-1.84 (m, 4H), 0.81 (br t, J=7.2 Hz, 3H); LCMS (ESI, M+1): m/z=712.3.

### Example 161

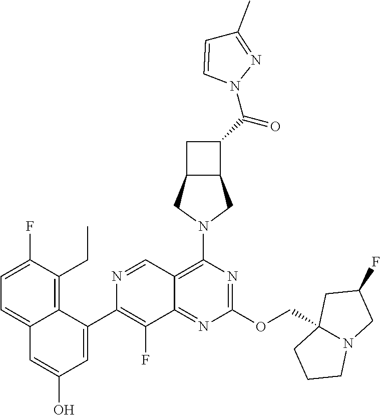 [SMILES: CCc1c(F)ccc2cc(O)cc(-c3ncc4c(N5C[C@H]6C[C@H](C(=O)n7ccc(C)n7)[C@H]6C5)nc(OC[C@@]56CCCN5C[C@H](F)C6)nc4c3F)c12]

### ((1S,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone

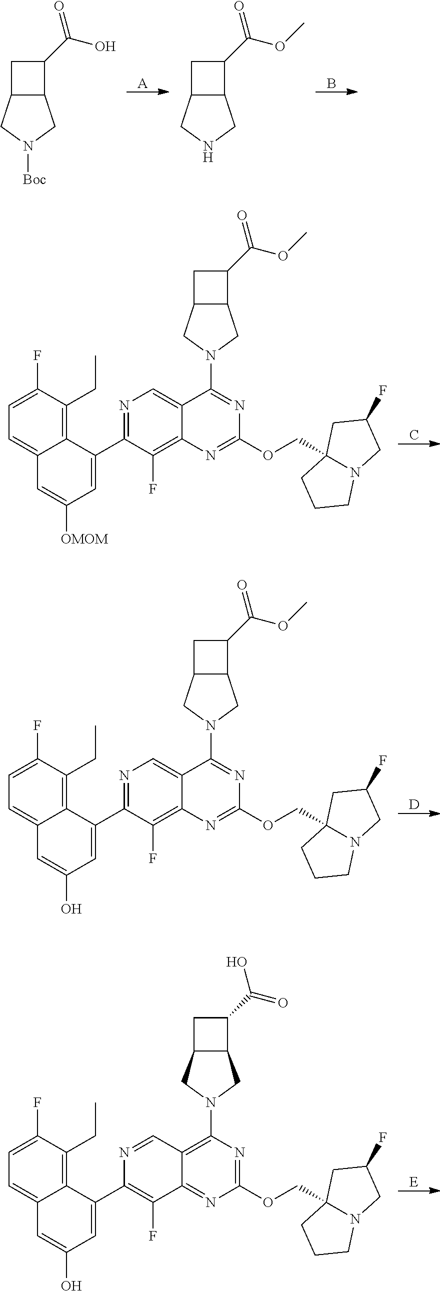

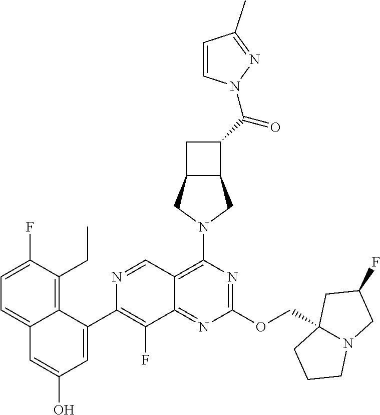 [SMILES: CCc1c(F)ccc2cc(O)cc(-c3ncc4c(N5C[C@H]6C[C@H](C(=O)n7ccc(C)n7)[C@H]6C5)nc(OC[C@@]56CCCN5C[C@H](F)C6)nc4c3F)c12]

Step A. methyl 3-azabicyclo[3.2.0]heptane-6-carboxylate: A solution of 3-(tert-butoxycarbonyl)-3-azabicyclo[3.2.0]heptane-6-carboxylic acid (1.35 g, 1.0 equiv) in HCl·MeOH (2 M, 10 mL) was stirred at 0 °C for 1 hour. The mixture was concentrated to afford the title compound (2.10 g, crude, HCl) as yellow oil.

Step B. methyl 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (1.20 g, 1.0 equiv) and methyl 3-azabicyclo[3.2.0]heptane-6-carboxylate (829 mg, 2.0 equiv, HCl) in DMSO (7 mL) were added benzotriazol-1-yloxyl-tris-(pyrrolidino)-phosphonium hexafluorophosphate (9.01 g, 8.0 equiv) and TEA (4.38 g, 20 equiv). The reaction was stirred at 30 °C for 60 hours. The mixture was filtered and purified with reversed phase flash chromatography [C18, 0.1% NH₃·H₂O condition] to afford the title compound (1.40 g, 83% yield) as yellow solid; LCMS (ESI, M+1): m/z=692.3.

Step C. methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate: To a solution of methyl 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate (1.40 g, 1.0 equiv) in MeCN (5 mL) was added HCl·dioxane (2 M, 10 mL). The reaction was stirred at 0 °C for 0.5 hours. The mixture was concentrated. The residue was diluted with water (5 mL). The mixture was adjusted to pH>8 with saturated Na₂CO₃ aqueous (10 mL) at 0 °C. The mixture was extracted with DCM (3×10 mL). The combined organic layers were washed with brine (20 mL), dried over anhydrous sodium sulfate, concentrated and purified with reversed phase flash chromatography [C18, 0.1% NH₃·H₂O condition] to afford the title compound (540 mg, 41% yield) as yellow solid; LCMS (ESI, M+1): m/z=648.4.

Step D. (1S,5S,6S)-3-(7-(8-ethyl-7-fluoro-3-hydroxynaphthalen-1-yl)-8-fluoro-2-yl)-3-azabicyclo[3.2.0]heptane-6-carboxylic acid: To a solution of methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate (540 mg, 1.0 equiv) in MeOH (3 mL) and THF (3 mL) was added LiOH·H₂O (105 mg, 3.0 equiv) in H₂O (1 mL). The reaction was stirred at 20 °C for 1 hour. The mixture was adjusted to pH≈6 with HCl (2 M) and extracted with EtOAc (3×10 mL). The combined organic layers were dried over anhydrous sodium sulfate, concentrated and purified with SFC [column: DAICEL CHIRALCEL OX (250 mm×30 mm, 10 μm); A: CO₂-ACN/MeOH (0.1% NH₃·H₂O); B %: 40%, isocratic elution mode] and [column: DAICEL CHIRALCEL OJ (250 mm×30 mm, 10 μm); A: CO₂-i-PrOH (0.1% NH₃·H₂O); B %: 40%, isocratic elution mode] to afford three peaks.

Peak 1:(1S,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (170 mg, 34% yield) as white solid; LCMS (ESI, M+1): m/z=634.2; SFC [Column: Chiralcel OX-3 50×4.6 mm I.D, 3 μm: Mobile phase: Phase A for CO₂ and Phase B for MeOH+ACN (0.05% DEA); Gradient elution: MeOH+ACN (0.05% DEA) in CO₂ from 30% to 60% Flow rate: 3 mL/min; Detector: PDA Column Temp: 35C; Back Pressure: 100Bar]. LCMS (ESI, M+1): m/z=634.4. RT=1.307 mins.

Peak 2: (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (130 mg, 26% yield) as white solid; LCMS (ESI, M+1): m/z=634.3; SFC [Column: Chiralcel OJ-3 50×4.6 mm I.D, 3 μm: Mobile phase: Phase A for CO₂ and Phase B IPA (0.05% DEA); Gradient elution: IPA (0.05% DEA) in CO₂ from 5% to 40% Flow rate: 3 mL/min; Detector: PDA Column Temp: 35C; Back Pressure: 100Bar]. LCMS (ESI, M+1): m/z=634.4. RT=2.252 mins

Peak 3: (1R,5R,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (20 mg, 4% yield) as white solid; LCMS (ESI, M+1): m/z=634.3; SFC [Column: Chiralcel OJ-3 50×4.6 mm I.D, 3 μm: Mobile phase: Phase A for CO₂ and Phase B IPA (0.05% DEA); IPA (0.05% DEA) in CO₂ from 5% to 40% Flow rate: 3 mL/min; Detector: PDA Column Temp: 35C; Back Pressure: 100Bar]. LCMS (ESI, M+1): m/z=634.4. RT=1.758 mins

Step E. ((1S,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone: To a solution of (1S,5S,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (100 mg, 1.0 equiv), DIEA (61.2 mg, 3.0 equiv) and HATU (90.0 mg, 1.5 equiv) in DMF (1 mL) was added 3-methyl-1H-pyrazole (38.9 mg, 3.0 equiv). The reaction was stirred at 20 °C for 12 hours. The mixture was filtered and purified with reversed phase flash chromatography [C18, neutral condition] to afford the title compound (8.11 mg, 6.9% yield) as off-white solid; ¹H NMR (400 MHz, DMSO-d₆) δ=9.94 (s, 1H), 9.35 (d, J=0.8 Hz, 1H), 8.29 (d, J=2.8 Hz, 1H), 7.79-7.75 (m, 1H), 7.40-7.28 (m, 2H), 7.03-7.00 (m, 1H), 6.45 (d, J=2.8 Hz, 1H), 5.40-5.16 (m, 1H), 4.72-4.70 (m, 1H), 4.53-4.37 (m, 1H), 4.26-4.05 (m, 4H), 4.01-3.95 (m, 1H), 3.47-3.40 (m, 1H), 3.13-3.10 (m, 3H), 2.88-2.79 (m, 1H), 2.42-2.13 (m, 7H), 2.13-1.99 (m, 3H), 1.95-1.66 (m, 4H), 0.74 (br t, J=6.4 Hz, 3H); LCMS (ESI, M+1): m/z=698.4.

### Example 162

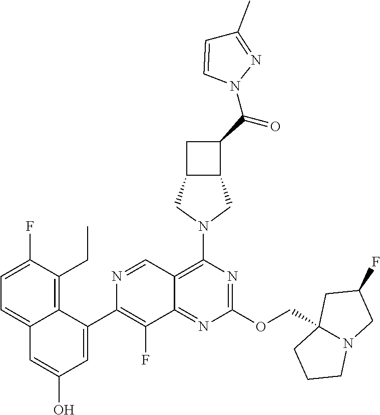 [SMILES: CCc1c(F)ccc2cc(O)cc(-c3ncc4c(N5C[C@@H]6C[C@@H](C(=O)n7ccc(C)n7)[C@@H]6C5)nc(OC[C@@]56CCCN5C[C@H](F)C6)nc4c3F)c12]

### ((1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone

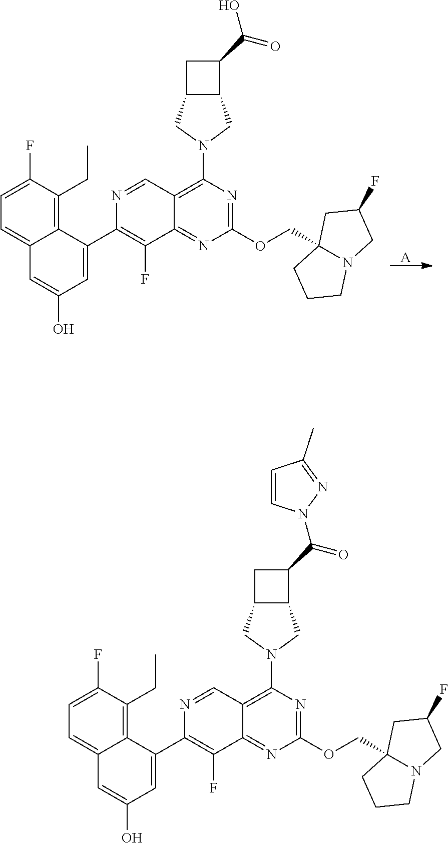

Step A. ((1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone: To a solution of (1R,5R,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (110 mg, 1.0 equiv), DIEA (67.3 mg, 3.0 equiv) and HATU (99.0 mg, 1.5 equiv) in DMF (1 mL) was added 3-methyl-1H-pyrazole (42.8 mg, 3.0 equiv). The reaction was stirred at 20 °C for 12 hours. The mixture was filtered and purified with reversed phase flash chromatography [C18, neutral condition] to afford the title compound (15.5 mg, 12% yield) as off-white solid; ¹H NMR (400 MHz, DMSO-d₆) δ=9.94 (s, 1H), 9.35 (s, 1H), 8.29 (d, J=2.4 Hz, 1H), 7.77 (dd, J=6.0, 9.2 Hz, 1H), 7.42-7.28 (m, 2H), 7.01 (dd, J=2.4, 8.8 Hz, 1H), 6.45 (d, J=2.8 Hz, 1H), 5.41-5.14 (m, 1H), 4.72-4.66 (m, 1H), 4.50-4.39 (m, 1H), 4.25-4.04 (m, 4H), 4.01-3.93 (m, 1H), 3.44-3.43 (m, 1H), 3.11-3.09 (m, 3H), 2.89-2.78 (m, 1H), 2.45-2.13 (m, 7H), 2.13-1.99 (m, 3H), 1.97-1.54 (m, 4H), 0.78-0.71 (m, 3H); LCMS (ESI, M+1): m/z=698.4.

### Example 163

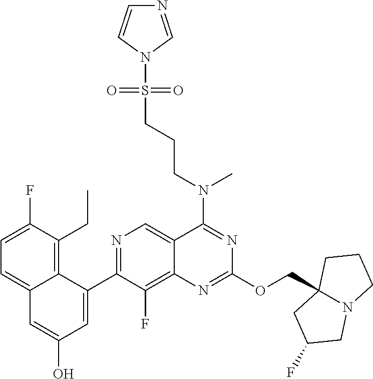 [SMILES: CCc1c(F)ccc2cc(O)cc(-c3ncc4c(N(C)CCCS(=O)(=O)n5ccnc5)nc(OC[C@@]56CCCN5C[C@H](F)C6)nc4c3F)c12]

### 4-(4-((3-((1H-imidazol-1-yl)sulfonyl)propyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

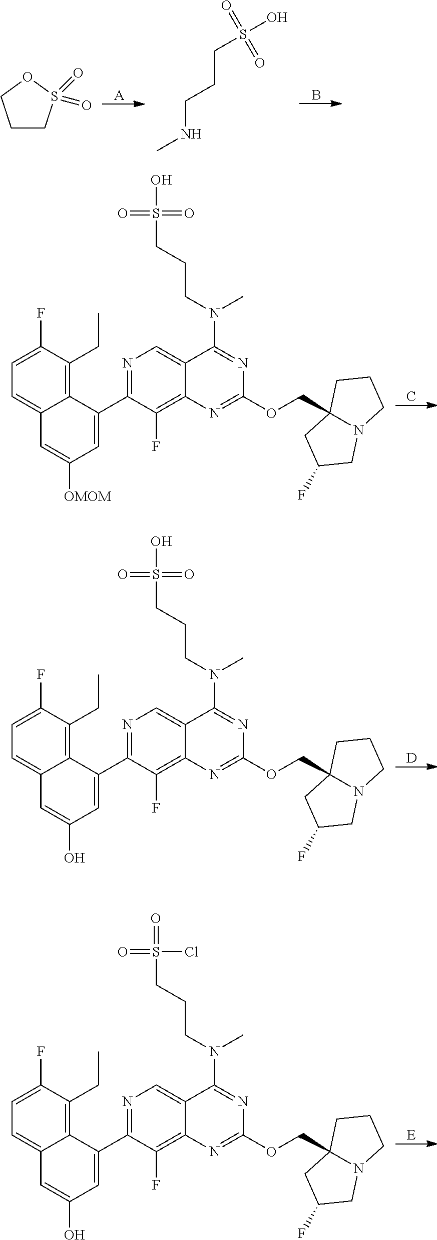

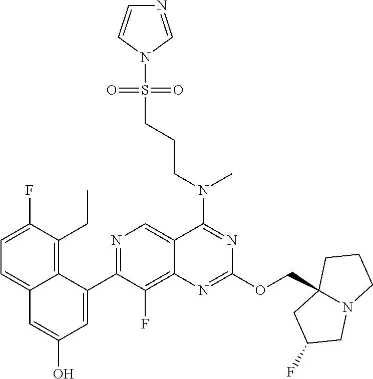 [SMILES: CCc1c(F)ccc2cc(O)cc(-c3ncc4c(N(C)CCCS(=O)(=O)n5ccnc5)nc(OC[C@@]56CCCN5C[C@H](F)C6)nc4c3F)c12]

Step A. 3-(methylamino)propane-1-sulfonic acid: To a solution of 1,2-oxathiolane 2,2-dioxide (10.0 g, 1.0 equiv) in THF (200 mL) was added MeNH₂ (2 M, 205 mL, 5.0 equiv) at 0 °C. The reaction was stirred at 20 °C for 12 hours. The mixture was concentrated and re-crystallization from MeOH (200 mL) at 60 °C for 0.5 hours to afford the title compound (6.00 g, 48% yield) as white solid; ¹H NMR (400 MHz, D₂O) δ=3.18-3.13 (m, 2H), 2.99-2.95 (m, 2H), 2.70 (s, 3H), 2.12-2.08 (m, 2H).

Step B. 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (1.50 g, 1.0 equiv), 3-(methylamino)propane-1-sulfonic acid (829 mg, 2.0 equiv) and TEA (1.37 g, 5.0 equiv) in DMSO (10 mL) was added benzotriazol-1-yloxyl-tris-(pyrrolidino)-phosphonium hexafluorophosphate (2.82 g, 2.0 equiv). The reaction was stirred at 20 °C for 12 hours. The mixture was filtered and purified by reversed phase flash [C18, 0.1% formic acid condition] to afford the title compound (1.60 g, 85% yield) as white solid; LCMS (ESI, M+1): m/z=690.2.

Step C. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid: To a solution of 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid (200 mg, 1.0 equiv) in DCM (1 mL) was added TFA (3.07 g, 93 equiv) at 0 °C. The reaction was stirred at 0 °C for 0.5 hours. The mixture was concentrated. The residue was diluted with water (1 mL). The mixture was adjusted to pH=7 with NaHCO₃ and extracted with EtOAc/MeOH=4:1 (2×10 mL). The combined organic layers were concentrated to afford the title compound (130 mg, 66% yield) as off-white solid; LCMS (ESI, M+1): m/z=646.3.

Step D. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonyl chloride: To a solution of 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid (100 mg, 1.0 equiv) in DCM (5 mL) was added (COCl)₂ (59.0 mg, 3.0 equiv) at 0 °C. The reaction was stirred at 0 °C for 5 mins. DMF (1.13 mg, 0.1 equiv) was added into the mixture. The reaction was stirred at 20 °C for 0.5 hours. The mixture was concentrated to afford the title compound (120 mg, crude) as yellow solid.

Step E. 4-(4-((3-((1H-imidazol-1-yl)sulfonyl)propyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a mixture of 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonyl chloride (100 mg, crude) and 1H-imidazole (51.2 mg, 5.0 equiv) in THF (6 mL) was added t-BuOK (50.7 mg, 3.0 equiv) at 0 °C. The reaction was stirred at 25 °C for 12 hours. The mixture was concentrated and purified by reversed phase flash [C18, H₂O/ACN condition] to afford the title compound (16.1 mg, 15% yield) as white solid; ¹H NMR (400 MHz, DMSO-d₆) δ=9.93 (s, 1H), 9.26 (s, 1H), 8.23 (s, 1H), 7.77 (dd, J=6.0, 9.2 Hz, 1H), 7.70 (t, J=1.2 Hz, 1H), 7.39-7.30 (m, 2H), 7.18 (s, 1H), 7.01 (d, J=2.4 Hz, 1H), 5.41-5.16 (m, 1H), 4.18-4.09 (m, 1H), 4.09-4.02 (m, 1H), 4.01-3.77 (m, 4H), 3.53 (s, 3H), 3.19-2.99 (m, 3H), 2.88-2.76 (m, 1H), 2.43-2.30 (m, 1H), 2.23-1.97 (m, 6H), 1.96-1.84 (m, 1H), 1.84-1.74 (m, 2H), 0.74 (t, J=7.2 Hz, 3H); LCMS (ESI, M+1): m/z=696.3.

### Example 164

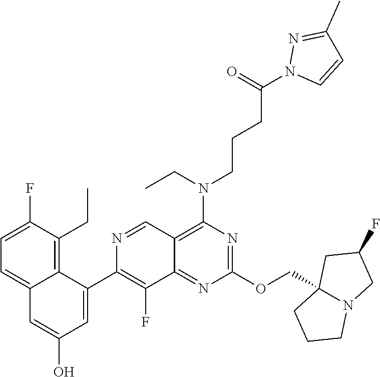 [SMILES: CCc1c(F)ccc2cc(O)cc(-c3ncc4c(N(CC)CCCC(=O)n5ccc(C)n5)nc(OC[C@@]56CCCN5C[C@H](F)C6)nc4c3F)c12]

### 4-(ethyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) butan-1-one

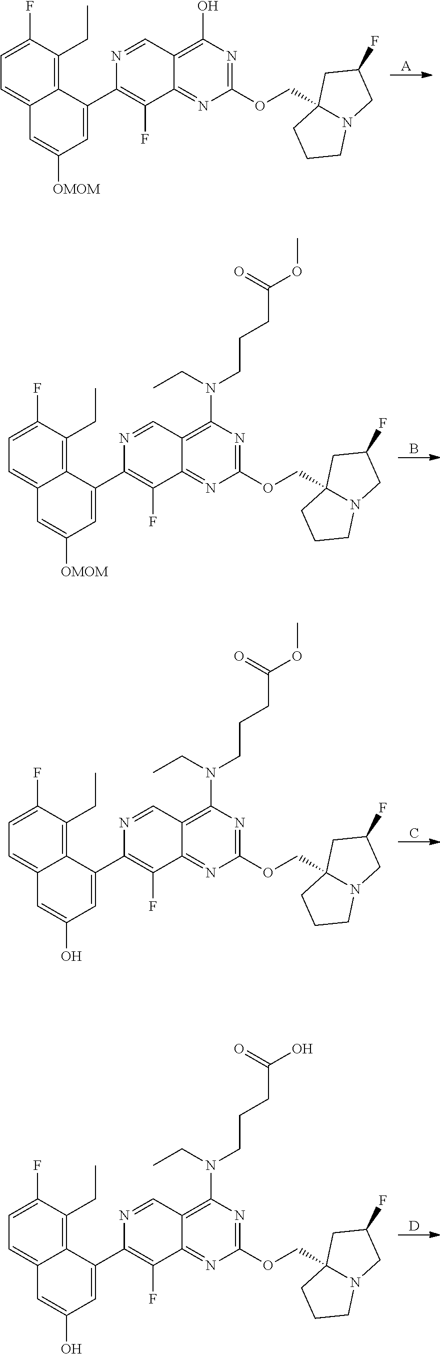

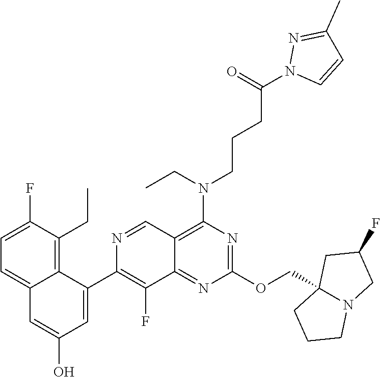 [SMILES: CCc1c(F)ccc2cc(O)cc(-c3ncc4c(N(CC)CCCC(=O)n5ccc(C)n5)nc(OC[C@@]56CCCN5C[C@H](F)C6)nc4c3F)c12]

Step A. methyl 4-(ethyl(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate: A mixture of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (300 mg, 1.0 equiv), methyl 4-(ethylamino)butanoate (147 mg, 1.5 equiv, HCl), DIEA (350 mg, 5.0 equiv) and PyBOP (422 mg, 1.5 equiv) in DMSO (3 mL) was stirred at 25 °C for 2 hours. The mixture was quenched by addition of water (10 mL) and extracted with ethyl acetate (3×10 mL). The combined organic layers were washed with saturated brine (3×15 mL), dried over anhydrous sodium sulfate, filtered, concentrated and purified by reversed-phase flash (0.1% FA condition) to afford the title compound (0.120 g, 32% yield) as yellow oil; LCMS (ESI, M+1): m/z=682.4.

Step B. methyl 4-(ethyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate: To a solution of methyl 4-(ethyl(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate (0.12 g, 1.0 equiv) in MeCN (1 mL) was added HCl·EtOAc (2 M, 2.00 mL) at 0 °C. The reaction was stirred at 25 °C for 2 hours. The mixture was concentrated, dissolved with MeOH (5 mL), adjusted to pH=7 with NaHCO₃solid, filtered and concentrated to afford the title compound (0.100 g, crude) as yellow oil; LCMS (ESI, M+1): m/z=638.3.

Step C. 4-(ethyl(7-(8-ethyl-7-fluoro-3-hydroxynaphthalen-1-yl)-8-fluoro-2-(((2R,7aR)-2-fluorotetrahydro-1H-pyrrolizin-7a(5H)-yl)methoxy)pyrido[4,3-d]pyrimidin-4-yl)amino)butanoic acid: To a solution of methyl 4-(ethyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate (100 mg, 1.0 equiv) in MeOH (2 mL) was added LiOH·H₂O (2 M, 2.00 mL, 26 equiv). The reaction was stirred at 25 °C for 0.5 hours. The mixture was filtered and purified by reversed-phase flash (0.1% FA condition) to afford the title compound (80.0 mg, 82% yield) as white solid; LCMS (ESI, M+1): m/z=624.4.

Step D. 4-(ethyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) butan-1-one: To a solution of 4-(ethyl(7-(8-ethyl-7-fluoro-3-hydroxynaphthalen-1-yl)-8-fluoro-2-(((2R,7aR)-2-fluorotetrahydro-1H-pyrrolizin-7a(5H)-yl)methoxy)pyrido[4,3-d]pyrimidin-4-yl)amino)butanoic acid (70.0 mg, 1.0 equiv) and 3-methyl-1H-pyrazole (27.7 mg, 3.0 equiv) in DMF (1 mL) were added HATU (51.2 mg, 1.2 equiv) and DIEA (43.5 mg, 3.0 equiv). The reaction was stirred at 25 °C for 1 hour. The mixture was filtered and purified by prep-HPLC [Waters Xbridge 150×25 mm×5 μm; A: water (NH₄HCO₃), B %: ACN, B %: 50%-80% over 15 min] to afford the title compound (10.2 mg, 13% yield) as yellow gum; ¹H NMR (400 MHz, CHLOROFORM-d) δ=8.98 (d, J=13.6 Hz, 1H), 8.11 (d, J=2.8 Hz, 1H), 7.53-7.41 (m, 1H), 7.19-7.00 (m, 3H), 6.24 (d, J=2.8 Hz, 1H), 5.47-5.14 (m, 1H), 4.39-4.21 (m, 2H), 3.93-3.73 (m, 5H), 3.40-3.29 (m, 2H), 3.28-3.18 (m, 3H), 3.17-3.06 (m, 1H), 3.04-2.92 (m, 1H), 2.59-2.33 (m, 2H), 2.31-2.29 (m, 3H), 2.27-2.21 (m, 2H), 2.17-2.08 (m, 2H), 2.02-1.94 (m, 2H), 1.42 (td, J=7.2, 10.4 Hz, 3H), 0.86-0.76 (m, 3H); LCMS (ESI, M+1): m/z=688.4.

### Example 165

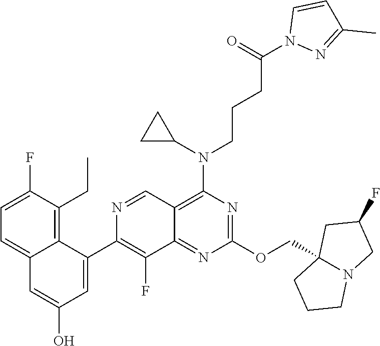 [SMILES: CCc1c(F)ccc2cc(O)cc(-c3ncc4c(N(CCCC(=O)n5ccc(C)n5)C5CC5)nc(OC[C@@]56CCCN5C[C@H](F)C6)nc4c3F)c12]

### 4-(cyclopropyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) butan-1-one

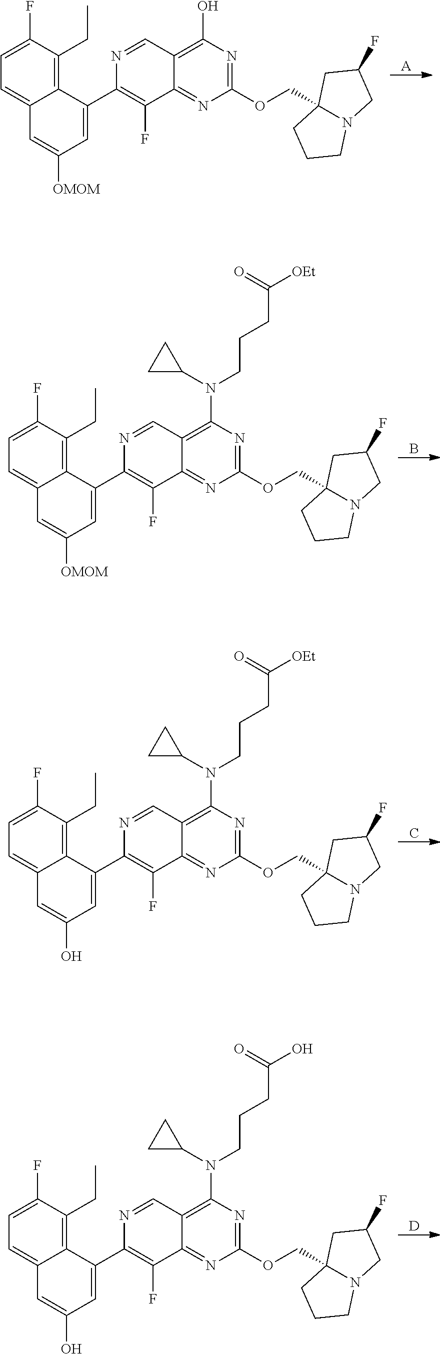

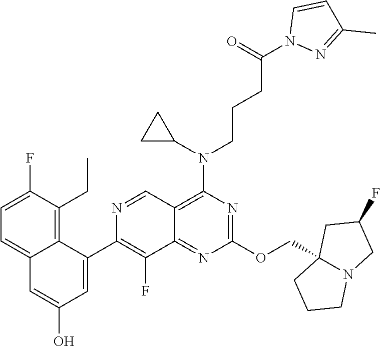 [SMILES: CCc1c(F)ccc2cc(O)cc(-c3ncc4c(N(CCCC(=O)n5ccc(C)n5)C5CC5)nc(OC[C@@]56CCCN5C[C@H](F)C6)nc4c3F)c12]

Step A. ethyl 4-(cyclopropyl(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (300 mg, 1.0 equiv), ethyl 4-(cyclopropylamino)butanoate (169 mg, 1.5 equiv, HCl) in DMSO (3 mL) were added DIEA (350 mg, 5.0 equiv) and PyBOP (422 mg, 1.5 equiv). The reaction was stirred at 25 °C for 2 hours. The mixture was quenched by addition of water (10 mL) and extracted with ethyl acetate (3×10 mL). The combined organic layers were washed with saturated brine (3×15 mL), dried over anhydrous sodium sulfate, filtered, concentrated and purified by reversed-phase flash (0.1% FA condition) to afford the title compound (0.130 g, 34% yield) as yellow oil; LCMS (ESI, M+1): m/z=708.4.

Step B. ethyl 4-(cyclopropyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate: To a solution of ethyl 4-(cyclopropyl(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate (130 mg, 1.0 equiv) in MeCN (1 mL) was added HCl·EtOAc (2 M, 2.00 mL) at 0 °C The reaction was stirred at 25 °C for 2 hours. The mixture was adjusted to pH=7 using NaHCO₃solid, filtered and concentrated to afford the title compound (0.100 g, crude) as yellow oil, LCMS (ESI, M+1): m/z=664.3.

Step C. 4-(cyclopropyl(7-(8-ethyl-7-fluoro-3-hydroxynaphthalen-1-yl)-8-fluoro-2-yl)amino)butanoic acid: To a solution of ethyl 4-(cyclopropyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoate (100 mg, 1.0 equiv) in MeOH (2 mL) was added LiOH·H₂O (2 M, 2.00 mL, 27 equiv). The reaction was stirred at 25 °C for 0.5 hours. The mixture was filtered and purified by reversed-phase flash (0.1% FA condition) to afford the title compound (80.0 mg, 83% yield) as white solid; LCMS (ESI, M+1): m/z=636.4.

Step D. 4-(cyclopropyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-1-(3-methyl-1H-pyrazol-1-yl) butan-1-one: To a solution of 4-(cyclopropyl(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)butanoic acid (70.0 mg, 1.0 equiv) and 3-methyl-1H-pyrazole (27.1 mg, 3.0 equiv) in DMF (1 mL) were added HATU (50.2 mg, 1.2 equiv) and DIEA (42.7 mg, 3.0 equiv). The reaction was stirred at 25 °C for 1 hour. The reaction mixture was filtered and purified by prep-HPLC [Waters Xbridge 150×25 mm×5 μm; A: water (NH₄HCO₃), B %: ACN, B %: 48%-78% over 15 min] to afford the title compound (40.5 mg, 51% yield) as off-white solid; ¹H NMR (400 MHz, CHLOROFORM-d) δ=9.49 (s, 1H), 8.13-8.03 (m, 1H), 7.49 (ddd, J=4.0, 5.6, 9.2 Hz, 1H), 7.19-7.01 (m, 3H), 6.17 (dd, J=2.8, 6.4 Hz, 1H), 5.42-5.16 (m, 1H), 4.33-4.18 (m, 3H), 3.94-3.77 (m, 1H), 3.40-3.31 (m, 1H), 3.29-3.04 (m, 5H), 3.02-2.90 (m, 1H), 2.55-2.26 (m, 4H), 2.24 (d, J=4.8 Hz, 3H), 2.22-2.07 (m, 3H), 2.00-1.77 (m, 3H), 1.20-1.01 (m, 2H), 0.88-0.62 (m, 5H); LCMS (ESI, M+1): m/z=700.4.

### Example 166

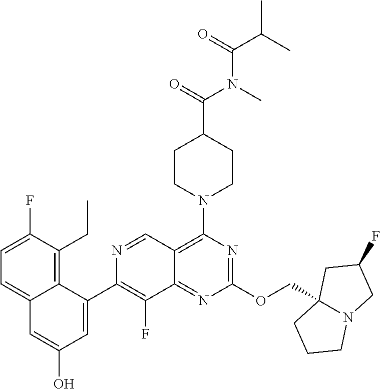 [SMILES: CCc1c(F)ccc2cc(O)cc(-c3ncc4c(N5CCC(C(=O)N(C)C(=O)C(C)C)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-isobutyryl-N-methylpiperidine-4-carboxamide

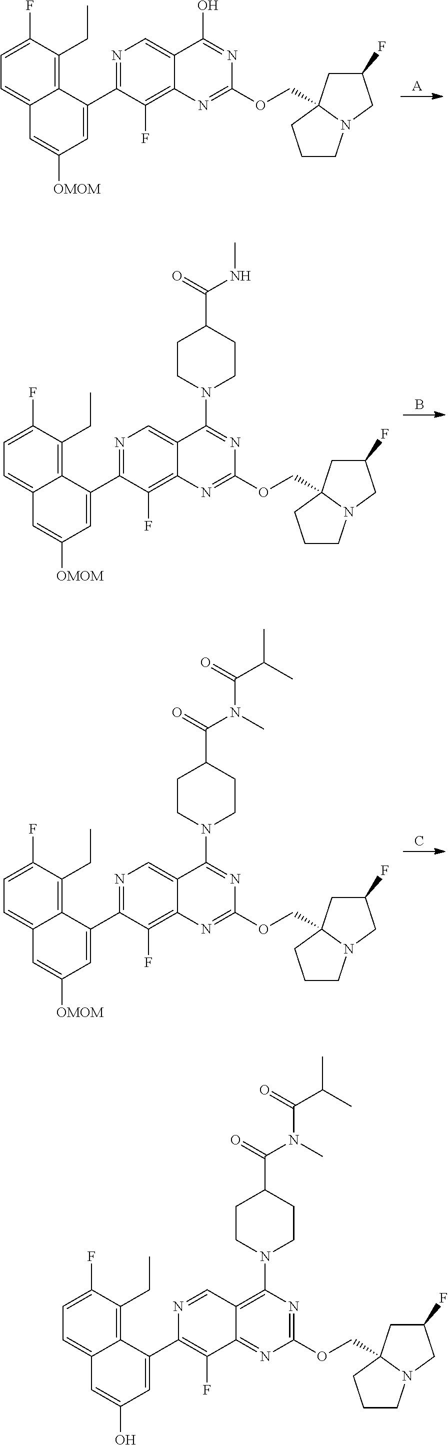

Step A. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (2 g, 1.0 equiv) and N-methylpiperidine-4-carboxamide (1.29 g, 2 equiv, HCl salt) in DMF (20 mL) were added PyBOP (2.82 g, 1.5 equiv) and DIEA (4.66 g, 10 equiv). The reaction was stirred at 25 °C for 1 hour. The mixture was filtered and the filtrate was purified by reversed phase flash chromatography [C18, 0.1% formic acid condition] to afford the title compound (1.56 g, 63% yield) as yellow solid; LCMS (ESI, M+1): m/z=679.4.

Step B. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-isobutyryl-N-methylpiperidine-4-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide (300 mg, 1.0 equiv) in THF (3 mL) was added NaH (53.0 mg, 60% purity, 3.0 equiv) at 0 °C. The reaction was stirred at 25 °C for 0.5 hours. To the above mixture was added a solution of isobutyryl chloride (70.6 mg, 1.5 equiv) in THF (3 mL) at 0 °C. The reaction was stirred at 25 °C for 0.5 hours. The mixture was quenched by addition H₂O (20 mL), extracted with ethyl acetate (3×8 mL), dried over Na₂SO₄, filtered and concentrated to afford the title compound (220 mg, crude) as yellow solid.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-isobutyryl-N-methylpiperidine-4-carboxamide: A mixture of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-isobutyryl-N-methylpiperidine-4-carboxamide (80 mg, 1.0 equiv) and HCl·dioxane (1.6 mL) was stirred at 0 °C for 15 minutes. The mixture was concentrated, basified with saturated NaHCO₃ solution (10 mL) and extracted with ethyl acetate (3×5 mL). The combined organic layers were dried over anhydrous sodium sulfate, concentrated and purified by prep-HPLC [Waters Xbridge 150×25 mm×5 μm; A: water (NH₄HCO₃), B: ACN, B %: 50%-80% over 9 min] to afford the title compound (4.26 mg, 5.53% yield) as white solid; ¹H NMR (400 MHz, CHLOROFORM-d) δ=8.96 (s, 1H), 7.57 (dd, J=5.6, 9.2 Hz, 1H), 7.24-7.16 (m, 2H), 7.04 (dd, J=2.4, 19.6 Hz, 1H), 5.41-5.18 (m, 1H), 4.69-4.57 (m, 2H), 4.33-4.17 (m, 2H), 3.62 (m, 1H), 3.48-3.33 (m, 2H), 3.29 (s, 3H), 3.27-3.05 (m, 3H), 3.04-2.89 (m, 1H), 2.57-2.43 (m, 1H), 2.36-1.77 (m, 12H), 1.24 (d, J=6.8 Hz, 6H), 0.83 (t, J=7.4 Hz, 3H); LCMS (ESI, M+1): m/z=705.4.

### Example 167

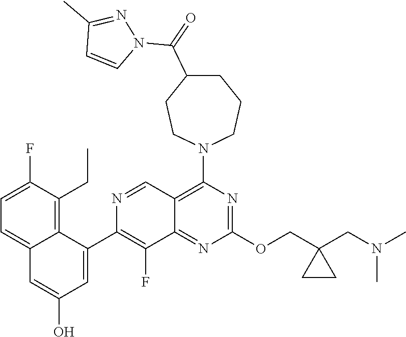 [SMILES: CCc1c(F)ccc2cc(O)cc(-c3ncc4c(N5CCCC(C(=O)n6ccc(C)n6)CC5)nc(OCC5(CN(C)C)CC5)nc4c3F)c12]

### (1-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl) azepan-4-yl)(3-methyl-1H-pyrazol-1-yl)methanone

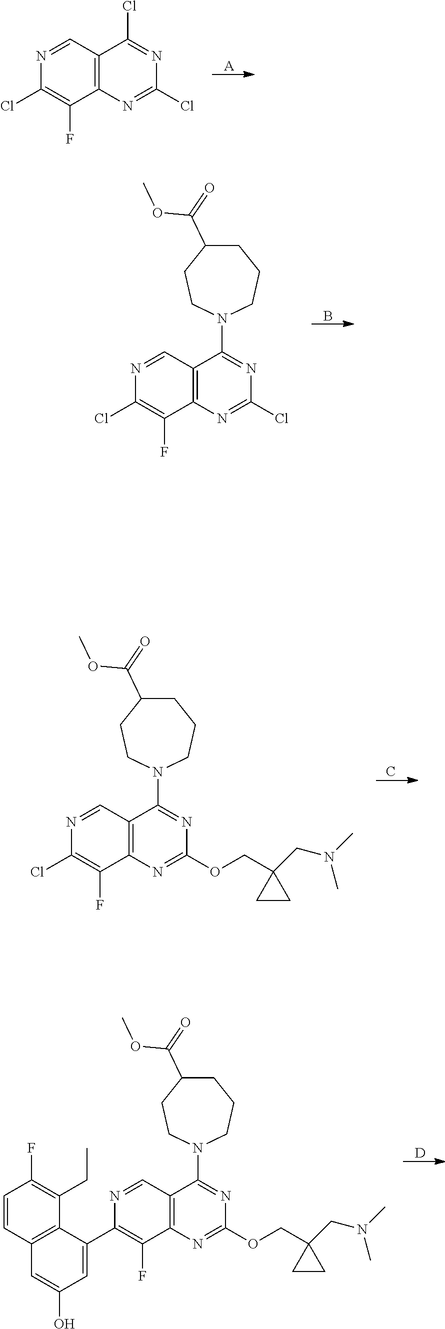

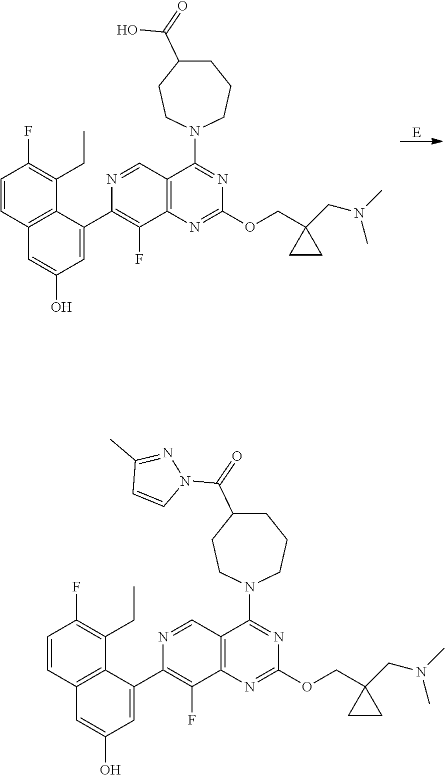

Step A. methyl 1-(2,7-dichloro-8-fluoropyrido[4,3-d]pyrimidin-4-yl)azepane-4-carboxylate: To a solution of 2,4,7-trichloro-8-fluoropyrido[4,3-d]pyrimidine (2 g, 1.0 equiv) in DCM (20 mL) were added DIEA (3.07 g, 3.0 equiv) and methyl azepane-4-carboxylate (1.12 g, 0.9 equiv). The reaction was stirred at −40 °C for 20 mintues. The mixture was diluted with water (30 mL), extracted with EtOAc (2×50 mL). The combined organic layers were washed with brine (2×5 mL), dried over anhydrous sodium sulfate, concentrated and purified by silica gel chromatography (petroleum ether/ethyl acetate 100/1 to 5/1) to afford the title compound (2.6 g, 78% yield) as red oil; LCMS (ESI, M+1): m/z=373.0.

Step B. methyl 1-(7-chloro-2-((1-((dimethylamino)methyl)cyclopropyl)methoxy)-8-fluoropyrido[4,3-d]pyrimidin-4-yl)azepane-4-carboxylate: To a mixture of methyl 1-(2,7-dichloro-8-fluoropyrido[4,3-d]pyrimidin-4-yl)azepane-4-carboxylate (1 g, 1.0 equiv) and (1-((dimethylamino)methyl)cyclopropyl)methanol (865 mg, 2.5 equiv) in dioxane (10 mL) was added DIEA (1.04 g, 3.0 equiv). The reaction was stirred at 90 °C for 1 hour. The mixture was diluted with water (50 mL), extracted with EtOAc (2×60 mL). The combined organic layers were washed with brine (2×10 mL), dried over anhydrous sodium sulfate, concentrated and purified by reversed phase flash chromatography [C18, 0.1% formic acid condition] to afford the title compound (700 mg, 55% yield) as yellow oil; LCMS (ESI, M+1): m/z=466.3.

Step C. methyl 1-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl)azepane-4-carboxylate: To a solution of methyl 1-(7-chloro-2-((1-((dimethylamino)methyl)cyclopropyl)methoxy)-8-fluoropyrido[4,3-d]pyrimidin-4-yl)azepane-4-carboxylate (300 mg, 1.0 equiv) and 5-ethyl-6-fluoro-4-(4,4,5,5-tetramethyl-1,3,2-dioxaborolan-2-yl)naphthalen-2-ol (305 mg, 1.5 equiv) in methoxycyclopentane (3 mL) were added K₃PO₄ (1.5 M, H₂O, 1.3 mL, 3.0 equiv) and CataCXium A Pd G3 (47 mg, 0.1 equiv). The reaction was degassed and purged with nitrogen 3 times. The reaction was stirred at 90 °C for 2 hours. The mixture was purified by reversed phase flash chromatography [C18, 0.1% formic acid condition] to afford the title compound (700 mg, 64% yield) as yellow solid; LCMS (ESI, M+1): m/z=620.4.

Step D. 1-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl)azepane-4-carboxylic acid: To a solution of methyl 1-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl)azepane-4-carboxylate (300 mg, 1.0 equiv) in MeOH (3 mL) were added LiOH·H₂O (2 M, H₂O, 0.1 mL, 4.0 equiv). The reaction was stirred at 25 °C for 15 mins. The mixture was filtered and purified by reversed phase flash chromatography [C18, 0.1% formic acid condition] to afford the title compound (250 mg, 78% yield) as white solid; LCMS (ESI, M+1): m/z=606.2.

Step E. (1-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl) azepan-4-yl)(3-methyl-1H-pyrazol-1-yl)methanone: To a mixture of 1-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl)azepane-4-carboxylic acid (100 mg, 1.0 equiv) and 3-methyl-1H-pyrazole (41 mg, 3.0 equiv) in DMF (1 mL) were added HATU (126 mg, 2.0 equiv) and DIEA (128 mg, 6.0 equiv). The reaction was stirred at 35 °C for 15 minutes. The mixture was filtered and purified by reversed phase flash [water/ACN] to afford the title compound (10.57 mg, 9% yield) as off-white solid; ¹H NMR (400 MHz, CHLOROFORM-d) δ=8.96 (d, J=13.6 Hz, 1H), 8.14 (d, J=2.8 Hz, 1H), 7.56-7.49 (m, 1H), 7.18 (t, J=9.2 Hz, 2H), 7.07 (dd, J=2.8, 12.4 Hz, 1H), 6.89-6.71 (m, 1H), 6.27 (d, J=2.8 Hz, 1H), 4.50-4.31 (m, 2H), 4.27-4.04 (m, 2H), 3.99-3.56 (m, 3H), 2.58-2.47 (m, 2H), 2.40-2.30 (m, 9H), 2.25-2.09 (m, 5H), 2.02-1.93 (m, 1H), 1.86-1.64 (m, 1H), 0.87-0.78 (m, 3H), 0.76-0.68 (m, 2H), 0.53 (br s, 2H); LCMS (ESI, M+1): m/z=670.5.

### Example 168

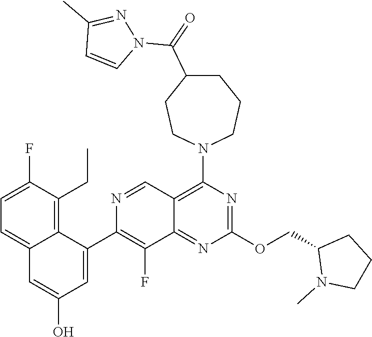 [SMILES: CCc1c(F)ccc2cc(O)cc(-c3ncc4c(N5CCCC(C(=O)n6ccc(C)n6)CC5)nc(OC[C@@H]5CCCN5C)nc4c3F)c12]

### (1-(7-(8-ethyl-7-fluoro-3-hydroxynaphthalen-1-yl)-8-fluoro-2-(((S)-1-methylpyrrolidin-2-yl)methoxy)pyrido[4,3-d]pyrimidin-4-yl) azepan-4-yl)(3-methyl-1H-pyrazol-1-yl)methanone

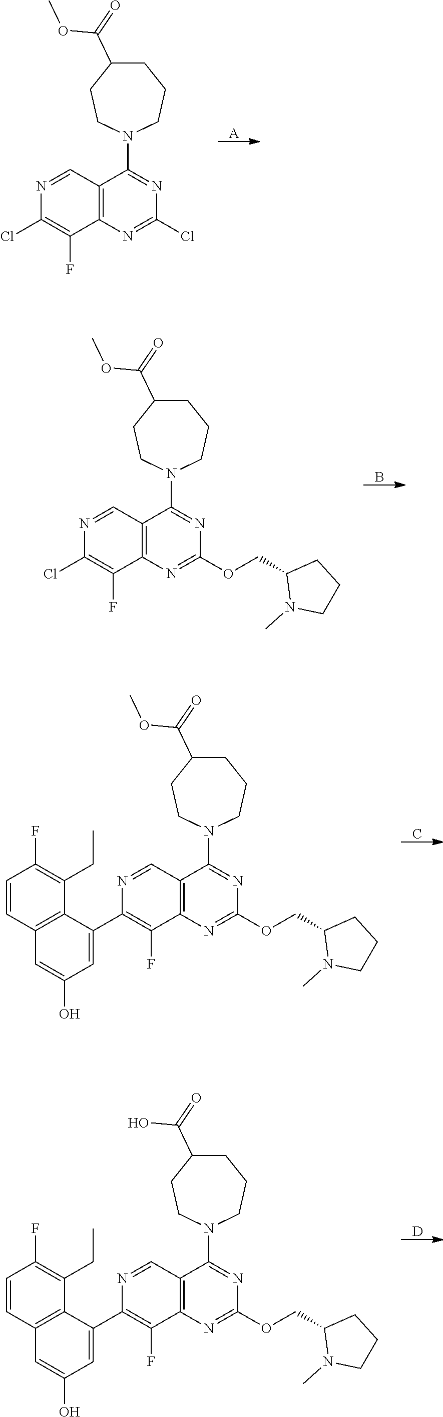

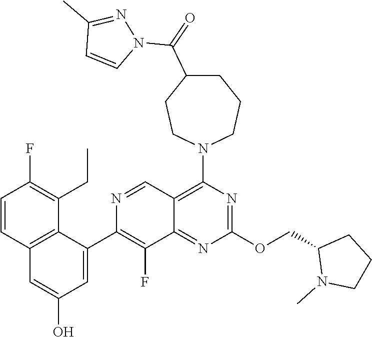 [SMILES: CCc1c(F)ccc2cc(O)cc(-c3ncc4c(N5CCCC(C(=O)n6ccc(C)n6)CC5)nc(OC[C@@H]5CCCN5C)nc4c3F)c12]

Step A. methyl 1-(7-chloro-8-fluoro-2-(((S)-1-methylpyrrolidin-2-yl)methoxy)pyrido[4,3-d]pyrimidin-4-yl)azepane-4-carboxylate: To a mixture of methyl 1-(2,7-dichloro-8-fluoropyrido[4,3-d]pyrimidin-4-yl)azepane-4-carboxylate (1 g, 1.0 equiv) and (S)-(1-methylpyrrolidin-2-yl)methanol (632 mg, 2.5 equiv) in dioxane (10 mL) was added DIEA (851 mg, 3.0 equiv). The reaction was stirred at 90 °C for 1.5 hours. The mixture was diluted with water (20 mL), extracted with EtOAc (2×40 mL). The combined organic layers were washed with brine (2×5 mL), dried over anhydrous sodium sulfate, concentrated and purified by reversed phase flash chromatography [C18, 0.1% formic acid condition] to afford the title compound (500 mg, 48% yield) as yellow solid; LCMS (ESI, M+1): m/z=452.2.

Step B. methyl 1-(7-(8-ethyl-7-fluoro-3-hydroxynaphthalen-1-yl)-8-fluoro-2-(((S)-1-methylpyrrolidin-2-yl)methoxy)pyrido[4,3-d]pyrimidin-4-yl)azepane-4-carboxylate: To a solution of methyl 1-(7-chloro-8-fluoro-2-(((S)-1-methylpyrrolidin-2-yl)methoxy)pyrido[4,3-d]pyrimidin-4-yl)azepane-4-carboxylate (200 mg, 1.0 equiv) and 5-ethyl-6-fluoro-4-(4,4,5,5-tetramethyl-1,3,2-dioxaborolan-2-yl)naphthalen-2-ol (210 mg, 1.5 equiv) in methoxycyclopentane (2 mL) were added K₃PO₄ (1.5 M, 0.8 mL, 3.0 equiv) and CataCXium A Pd G3 (32 mg, 0.1 equiv). The reaction was degassed and purged with nitrogen 3 times. The reaction was stirred at 90 °C for 2 hours. The mixture was diluted with water (30 mL), extracted with EtOAc (2×50 mL). The combined organic layers were washed with brine (2×5 mL), dried over anhydrous sodium sulfate, concentrated and purified by reversed phase flash chromatography [C18, 0.1% formic acid condition] to afford the title compound (400 mg, 71% yield) as red solid; LCMS (ESI, M+1): m/z=606.3.

Step C. 1-(7-(8-ethyl-7-fluoro-3-hydroxynaphthalen-1-yl)-8-fluoro-2-(((S)-1-methylpyrrolidin-2-yl)methoxy)pyrido[4,3-d]pyrimidin-4-yl)azepane-4-carboxylic acid: To a solution of methyl 1-(7-(8-ethyl-7-fluoro-3-hydroxynaphthalen-1-yl)-8-fluoro-2-(((S)-1-methylpyrrolidin-2-yl)methoxy)pyrido[4,3-d]pyrimidin-4-yl)azepane-4-carboxylate (350 mg, 1.0 equiv) in MeOH (3.5 mL) was added LiOH·H₂O (2 M, 1 mL, 4.0 equiv). The reaction was stirred at 25 °C for 20 mins. The mixture was filtered and purified by reversed phase flash chromatography [C18, 0.1% formic acid condition] to afford the title compound (250 mg, 67% yield) as white solid; LCMS (ESI, M+1): m/z=592.3.

Step D. (1-(7-(8-ethyl-7-fluoro-3-hydroxynaphthalen-1-yl)-8-fluoro-2-(((S)-1-methylpyrrolidin-2-yl)methoxy)pyrido[4,3-d]pyrimidin-4-yl) azepan-4-yl)(3-methyl-1H-pyrazol-1-yl)methanone: To a mixture of 1-(7-(8-ethyl-7-fluoro-3-hydroxynaphthalen-1-yl)-8-fluoro-2-(((S)-1-methylpyrrolidin-2-yl)methoxy)pyrido[4,3-d]pyrimidin-4-yl)azepane-4-carboxylic acid (100 mg, 1.0 equiv) and 3-methyl-1H-pyrazole (42 mg, 3.0 equiv) in DMF (1 mL) were added HATU (128 mg, 2.0 equiv) and DIEA (131 mg, 6.0 equiv). The reaction was stirred at 35 °C for 20 minutes. The mixture was filtered and purified by reversed phase flash [water/ACN] to afford the title compound (24.3 mg, 21% yield) as yellow solid; ¹H NMR (400 MHz, CHLOROFORM-d) δ=9.09-9.00 (m, 1H), 8.12 (br s, 1H), 7.48-7.38 (m, 1H), 7.17-7.01 (m, 3H), 6.26 (br s, 1H), 4.64-4.51 (m, 1H), 4.42-4.17 (m, 3H), 4.02-3.73 (m, 3H), 3.13 (br t, J=6.8 Hz, 1H), 2.76 (br d, J=4.4 Hz, 1H), 2.50 (br d, J=3.6 Hz, 4H), 2.43-2.27 (m, 5H), 2.18 (br s, 4H), 2.06 (s, 2H), 1.92-1.70 (m, 4H), 0.90-0.78 (m, 3H); LCMS (ESI, M+1): m/z=656.3.

### Example 169

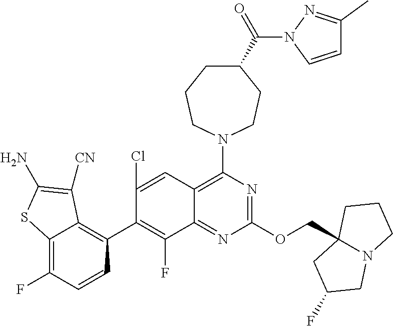

### 2-amino-4-((R)-6-chloro-8-fluoro-2-(((2R,7aS)-2-fluorotetrahydro-1H-pyrrolizin-7a(5H)-yl)methoxy)-4-((S)-4-(3-methyl-1H-pyrazole-1-carbonyl) azepan-1-yl)quinazolin-7-yl)-7-fluorobenzo [b]thiophene-3-carbonitrile

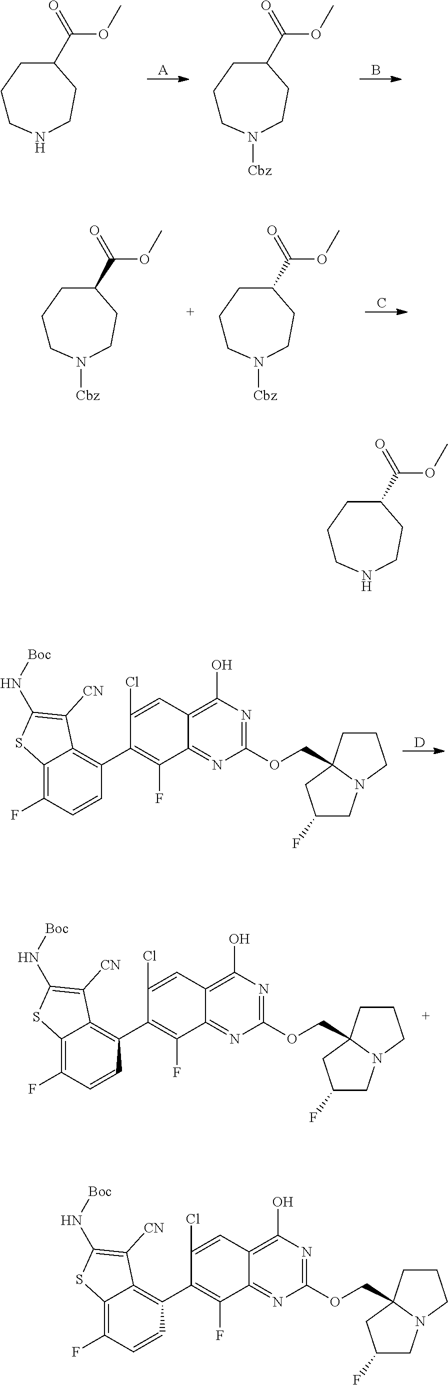

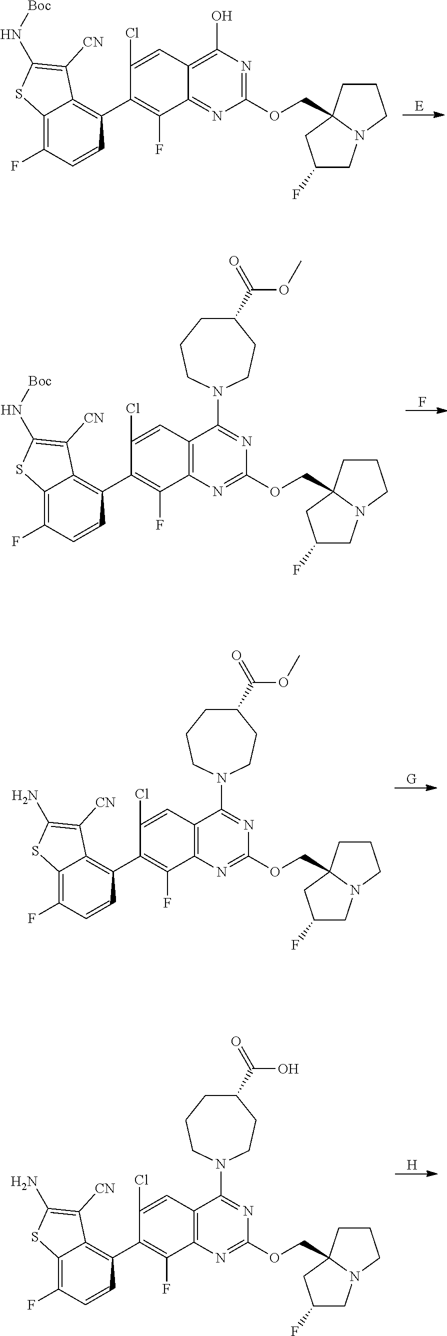

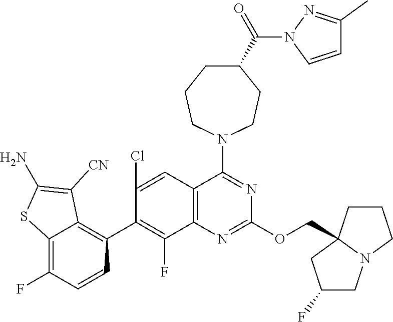

Step A 1-benzyl 4-methyl azepane-1,4-dicarboxylate: To a solution of methyl azepane-4-carboxylate hydrogen chloride (3.50 g, 1.0 equiv) in THF (25 mL) and H₂O (10 mL) were added NaHCO₃ (6.07 g, 4.0 equiv) and CbzCl (4.62 g, 1.5 equiv) at 0 °C. The reaction was stirred at 20 °C for 1 hour. The mixture was diluted with water (50 mL) and extracted with EtOAc (4×20 mL). The combined organic layers were washed with brine (50 mL), dried over anhydrous sodium sulfate, concentrated and purified with reversed phase flash [water (0.1% formic acid)/acetonitrile] to afford the title compound (5.19 g, 96% yield) as colorless liquid; LCMS (ESI, M+1): m/z=292.2.

Step B 1-benzyl 4-methyl (R)-azepane-1,4-dicarboxylate and 1-benzyl 4-methyl (S)-azepane-1,4-dicarboxylate: 1-benzyl 4-methyl azepane-1,4-dicarboxylate (5.19 g) was purified with SFC [Column: Chiralpak AY-3×50×4.6 mm I.D., 3 μm; Mobile phase: Phase A for CO₂ and Phase B for IPA (0.05% DEA); Gradient elution: IPA (0.05% DEA) in CO₂ from 5% to 40%; Flow rate: 3 mL/minute; Detector: PDA; Column Temp: 35C; Back Pressure: 100Bar] to afford two peaks

Peak 1:1-benzyl 4-methyl (R)-azepane-1,4-dicarboxylate (2.48 g, 47% yield) as colorless liquid; HPLC>98%, [Column: Chiralpak AY-3×50×4.6 mm I.D., 3 μm; Mobile phase: Phase A for CO₂ and Phase B for IPA (0.05% DEA); Gradient elution: IPA (0.05% DEA) in CO₂ from 5% to 40%; Flow rate: 3 mL/minute; Detector: PDA; Column Temp: 35 °C; Back Pressure: 100 Bar]. RT=1.195 minutes

Peak 2:1-benzyl 4-methyl (S)-azepane-1,4-dicarboxylate: (2.46 g, 47% yield) as colourless liquid; HPLC>99%, [Column: Chiralpak AY-3×50×4.6 mm I.D., 3 μm; Mobile phase: Phase A for CO₂ and Phase B for IPA (0.05% DEA); Gradient elution: IPA (0.05% DEA) in CO₂ from 5% to 40%; Flow rate: 3 mL/minute; Detector: PDA; Column Temp: 35 °C; Back Pressure: 100 Bar]. RT=1.530 minutes

Step C. methyl (S)-azepane-4-carboxylate: To a solution of 1-benzyl 4-methyl (S)-azepane-1,4-dicarboxylate (2.0 g, 1.0 equiv) in MeOH (20 mL) was added Pd/C (500 mg, 10% purity) under N₂ atmosphere. The suspension was degassed and purged with H₂ for 3 times. The reaction was stirred at 20 °C for 1 hour under H₂ (15 psi) atmosphere. The mixture was filtered through a pad of Celite and concentrated to afford the title compound (1.26 g, 72% yield) as yellow oil; LCMS (ESI, M+1): m/z=158.3.

Step D. tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate and tert-butyl(4-((R)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate: tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (4.0 g, 1.0 equiv) was separated with SFC [Column: Chiralcel OX-3×50×4.6 mm I.D., 3 μm; Mobile phase: Phase A for CO₂ and Phase B for IPA+acetonitrile (0.05% DEA); Gradient elution: IPA+acetonitrile (0.05% DEA) in CO₂ from 20% to 60%; Flow rate: 3 mL/minute; Detector: PDA; Column Temp: 35 °C; Back Pressure: 100 Bar] to afford two peaks:

Peak 1: tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (2.10 g, 49% yield) as yellow solid; HPLC>99%, [Column: Chiralcel OX-3×50×4.6 mm I.D., 3 μm; Mobile phase: Phase A for CO₂ and Phase B for IPA+acetonitrile (0.05% DEA); Gradient elution: IPA+acetonitrile (0.05% DEA) in CO₂ from 20% to 60%; Flow rate: 3 mL/minute; Detector: PDA; Column Temp: 35 °C; Back Pressure: 100 Bar]; LCMS (ESI, M+1): m/z=646.2. RT=1.103 minutes

Peck 2: tert-butyl(4-((R)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (1.90 g, 47% yield) as yellow solid; HPLC>98%, [Column: Chiralcel OX-3×50×4.6 mm I.D., 3 μm; Mobile phase: Phase A for CO₂ and Phase B for IPA+acetonitrile (0.05% DEA); Gradient elution: IPA+acetonitrile (0.05% DEA) in CO₂ from 20% to 60%; Flow rate: 3 mL/minute; Detector: PDA; Column Temp: 35 °C; Back Pressure: 100 Bar]; LCMS (ESI, M+1): m/z=646.1. RT=2.123 minutes

Step E. methyl (S)-1-((S)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (700 mg, 1.0 equiv) and TEA (438 mg, 4.0 equiv) in DMSO (7 mL) were added (Benzotriazol-1-yloxy)tripyrrolidinophosphonium hexafluorophosphate (1.13 g, 2.0 equiv) and methyl (S)-azepane-4-carboxylate (510 mg, 3.0 equiv). The reaction was stirred at 25 °C for 14 hours. The mixture was purified with reversed phase flash chromatography [water (0.1% NH₃·H₂O)/acetonitrile=1/4] to afford the title compound (422 mg, 47% yield) as yellow solid; LCMS (ESI, M+1): m/z=785.3.

Step F. methyl (S)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl (S)-1-((S)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (400 mg, 1.0 equiv) in DCM (4 mL) was added TFA (6.0 mL) at 0 °C. The reaction was stirred at 20 °C for 1 hour. The mixture was concentrated. The residue was dissolved in EtOAc (25 mL) and saturated NaHCO₃ aqueous (25 mL) at 0 °C. The mixture was extracted with EtOAc (4×20 mL). The combined organic layers were washed with brine (25 mL), dried over anhydrous sodium sulfate and concentrated to afford the title compound (348 mg, 95% yield) as yellow solid; LCMS (ESI, M+1): m/z=685.2

Step G. (S)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl (S)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (328 mg, 1.0 equiv) in MeOH (3.5 mL) was added LiOH·H₂O (1.5 M in H₂O, 1.0 mL, 3.0 equiv). The reaction was stirred at 20 °C for 2 hours. The mixture was adjusted to pH=7 with saturated HCl (1 M, 1.5 mL) at 0 °C. The mixture was purified with reversed phase flash chromatography [water (0.1% formic acid)/acetonitrile=1/1] to afford the title compound (238 mg, 74% yield) as yellow solid; LCMS (ESI, M+1): m/z=671.2.

Step H. 2-amino-4-((S)-6-chloro-8-fluoro-2-(((2R,7aS)-2-fluorotetrahydro-1H-pyrrolizin-7a(5H)-yl)methoxy)-4-((S)-4-(3-methyl-1H-pyrazole-1-carbonyl) azepan-1-yl)quinazolin-7-yl)-7-fluorobenzo[b]thiophene-3-carbonitrile: To a solution of (S)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (218 mg, 1.0 equiv) and 3-methyl-1H-pyrazole (67.2 mg, 2.5 equiv) in DMAc (2 mL) were added TEA (148 mg, 4.5 equiv), EDCI (112 mg, 1.8 equiv) and HOBt (83.4 mg, 1.9 equiv). The reaction was stirred at 20 °C for 14 hours. The mixture was purified with reversed phase flash [water/acetonitrile=1/1] to afford the title compound (87.2 mg, 35% yield) as white solid; ¹H NMR (400 MHz, DMSO-d6) δ=8.28 (d, J=2.8 Hz, 1H), 8.10 (s, 2H), 7.99 (s, 1H), 7.26 (dd, J=5.2, 8.4 Hz, 1H), 6.46 (d, J=2.8 Hz, 1H), 5.36-5.18 (m, 1H), 4.26-4.05 (m, 3H), 4.05-3.97 (m, 1H), 3.97-3.71 (m, 3H), 3.17-2.96 (m, 3H), 2.87-2.76 (m, 1H), 2.34-2.25 (m, 3H), 2.25-1.91 (m, 8H), 1.89-1.72 (m, 3H), 1.71-1.62 (m, 1H); LCMS (ESI, M+1): m/z=735.2.

### Example 170

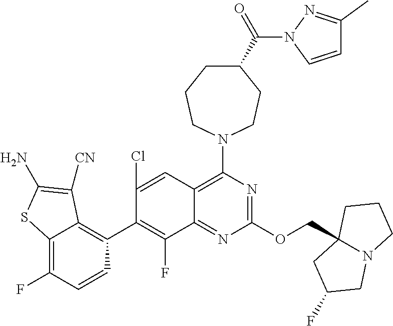

### 2-amino-4-((S)-6-chloro-8-fluoro-2-(((2R,7aS)-2-fluorotetrahydro-1H-pyrrolizin-7a(5H)-yl)methoxy)-4-((S)-4-(3-methyl-1H-pyrazole-1-carbonyl) azepan-1-yl)quinazolin-7-yl)-7-fluorobenzo[b]thiophene-3-carbonitrile

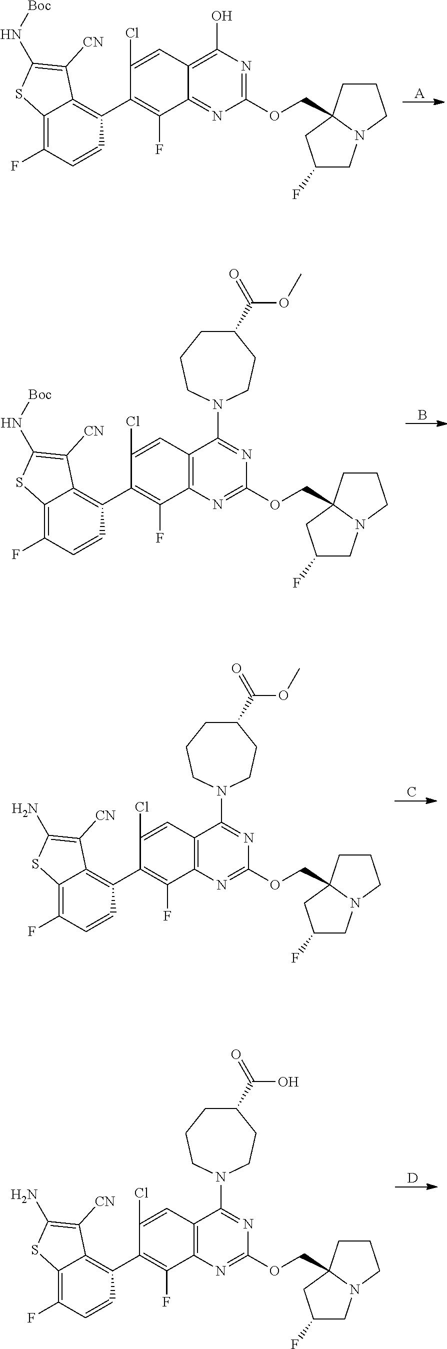

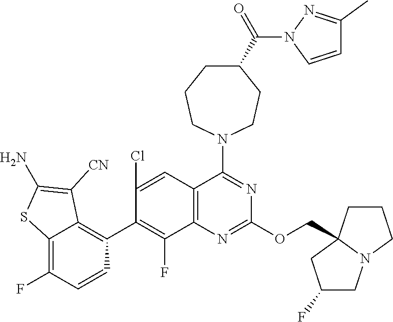

Step A. methyl (S)-1-((R)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of tert-butyl(4-((R)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (700 mg, 1.0 equiv) and TEA (438 mg, 4.0 equiv) in DMSO (7 mL) were added (Benzotriazol-1-yloxy)tripyrrolidinophosphonium hexafluorophosphate (1.13 g, 2.0 equiv) and methyl (S)-azepane-4-carboxylate (511 mg, 3.0 equiv). The reaction was stirred at 25 °C for 14 hours. The mixture was purified with reversed phase flash chromatography [water (0.1% NH₃·H₂O)/acetonitrile=1/4] to afford the title compound (390 mg, 44% yield) as yellow solid; LCMS (ESI, M+1): m/z=785.3.

Step B. methyl (S)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl (S)-1-((R)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (370 mg, 1.0 equiv) in DCM (4 mL) was added TFA (6 mL) at 0 °C. The reaction was stirred at 20 °C for 1 hour. The mixture was concentrated. The residue was dissolved in EtOAc (20 mL) and saturated Na₂CO₃ aqueous (20 mL) at 0 °C. The mixture was extracted with EtOAc (4×15 mL). The combined organic layers were washed brine (20 mL), dried over anhydrous sodium sulfate and concentrated to afford the title compound (350 mg, 95% yield) as yellow solid; LCMS (ESI, M+1): m/z=685.2.

Step C. (S)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl (S)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (330 mg, 1.0 equiv) in MeOH (3.5 mL) was added LiOH·H₂O (1.5 M in H₂O, 1 mL, 3.0 equiv). The reaction was stirred at 20 °C for 2 hours. The mixture was adjusted to pH=7 with 1 N HCl at 0 °C and concentrated. The residue was purified with reversed phase flash chromatography [water (0.1% formic acid)/acetonitrile=1/1] to afford the title compound (233 mg, 72% yield) as yellow solid; LCMS (ESI, M+1): m/z=671.2.

Step D. 2-amino-4-((R)-6-chloro-8-fluoro-2-(((2R,7aS)-2-fluorotetrahydro-1H-pyrrolizin-7a(5H)-yl)methoxy)-4-((S)-4-(3-methyl-1H-pyrazole-1-carbonyl) azepan-1-yl)quinazolin-7-yl)-7-fluorobenzo[b]thiophene-3-carbonitrile: To a solution of (S)-1-((R)-7-fluorotetrahydro-1H-pyrrolizin-7a(5H)-yl)methoxy)quinazolin-4-yl)azepane-4-carboxylic acid (213 mg, 1.0 equiv) and 3-methyl-1H-pyrazole (73.0 mg, 2.5 equiv) in DMAc (2 mL) were added TEA (144 mg, 4.5 equiv), EDCI (109 mg, 1.8 equiv) and HOBt (81.5 mg, 1.9 equiv). The reaction was stirred at 20 °C for 14 hours. The mixture was purified with reversed phase flash chromatography [water/acetonitrile=1/1] to afford the title compound (98.2 mg, 40% yield) as white solid; ¹H NMR (400 MHz, DMSO-d6) δ=8.27 (d, J=2.8 Hz, 1H), 8.11 (s, 2H), 8.04-7.95 (m, 1H), 7.27-7.20 (m, 1H), 7.19-7.10 (m, 1H), 6.45 (d, J=2.8 Hz, 1H), 5.39-5.18 (m, 1H), 4.29-4.09 (m, 3H), 4.08-3.98 (m, 1H), 3.95-3.71 (m, 3H), 3.22-2.97 (m, 3H), 2.91-2.76 (m, 1H), 2.34-2.21 (m, 4H), 2.20-1.95 (m, 7H), 1.90-1.73 (m, 3H), 1.71-1.59 (m, 1H); LCMS (ESI, M+1): m/z=735.2.

### Example 171

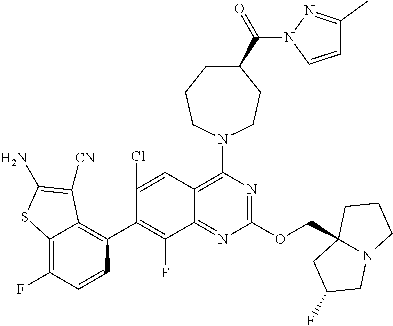

### 2-amino-4-((R)-6-chloro-8-fluoro-2-(((2R,7aS)-2-fluorotetrahydro-1H-pyrrolizin-7a(5H)-yl)methoxy)-4-((R)-4-(3-methyl-1H-pyrazole-1-carbonyl) azepan-1-yl)quinazolin-7-yl)-7-fluorobenzo[b]thiophene-3-carbonitrile

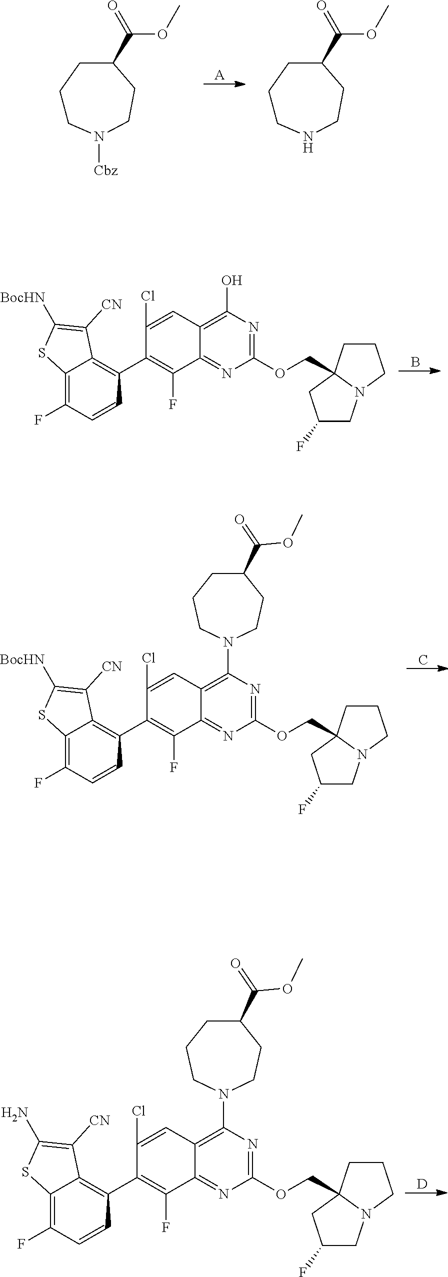

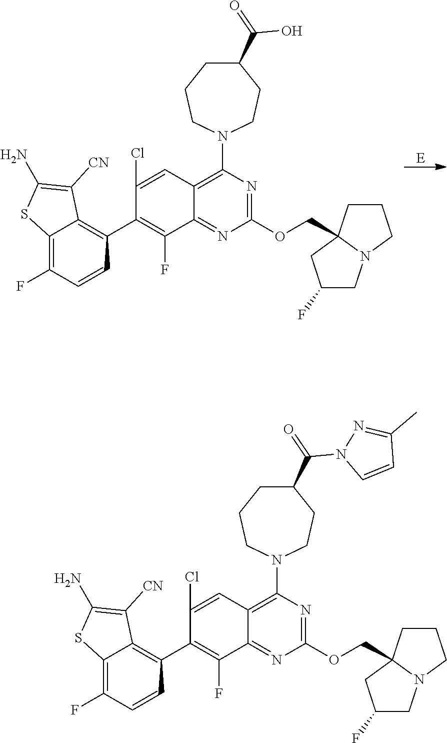

Step A. methyl (R)-azepane-4-carboxylate: To a solution of 1-benzyl 4-methyl (R)-azepane-1,4-dicarboxylate (2.00 g, 1.0 equiv) in MeOH (20 mL) was added Pd/C (231 mg, 10% purity). The reaction was degassed and purged with H₂ 3 times. The mixture was stirred under H₂ (15 Psi) at 20 °C for 1 hour. The mixture was filtered and concentrated to afford the title compound (1.20 g, crude) as yellow oil.

Step B. methyl (R)-1-((S)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (700 mg, 1.00 equiv) and methyl (R)-azepane-4-carboxylate (511 mg, 3.0 equiv) in DMSO (7 mL) were added benzotriazol-1-yloxyl-tris-(pyrrolidino)-phosphonium hexafluorophosphate (2.82 g, 5.0 equiv) and TEA (329 mg, 3.0 equiv). The reaction was stirred at 30 °C for 48 hours. The mixture was filtered and purified with reversed phase flash chromatography [C18, 0.1% formic acid condition] to afford the title compound (300 mg 33% yield) as yellow solid; LCMS (ESI, M+1): m/z=785.3.

Step C. methyl (R)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl (R)-1-((S)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (300 mg, 1.0 equiv) in DCM (2 mL) was added TFA (4 mL). The reaction was stirred at 20 °C for 0.5 hours. The mixture was adjusted to pH>8 with saturated Na₂CO₃ aqueous (10 mL) at 0 °C and extracted with DCM (3×5 mL). The combined organic layers were dried over anhydrous sodium sulfate and concentrated to afford the title compound (250 mg, crude) as yellow solid; LCMS (ESI, M+1): m/z=685.2.

Step D. (R)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl (R)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (250 mg, 1.0 equiv) in MeOH (2 mL) and THF (1 mL) was added a solution of LiOH·H₂O (26.2 mg, 3.0 equiv) in H₂O (0.5 mL). The reaction was stirred at 20 °C for 1 hour. The mixture was diluted with HCl (4 M, 5 mL) and purified with reversed phase flash chromatography [C18, 0.1% formic acid condition] to afford the title compound (150 mg, 61% yield) as yellow solid; LCMS (ESI, M+1): m/z=671.2.

Step E. 2-amino-4-((S)-6-chloro-8-fluoro-2-(((2R,7aS)-2-fluorotetrahydro-1H-pyrrolizin-7a(5H)-yl)methoxy)-4-((R)-4-(3-methyl-1H-pyrazole-1-carbonyl) azepan-1-yl)quinazolin-7-yl)-7-fluorobenzo[b]thiophene-3-carbonitrile: To a mixture of (R)-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (140 mg, 1.0 equiv), 3-methyl-1H-pyrazole (51.4 mg, 3.0 equiv) and TEA (63.3 mg, 3.0 equiv) in DMAC (1 mL) were added HOBt (42.3 mg, 1.5 equiv) and EDCI (60.0 mg, 1.5 equiv). The reaction was stirred at 20 °C for 12 hours. The mixture was filtered and purified with reversed phase flash chromatography [C18, neutral condition] to afford the title compound (53.8 mg, 32% yield) as off-white solid; ¹H NMR (400 MHz, DMSO-d6) δ=8.27 (d, J=2.8 Hz, 1H), 8.10 (s, 2H), 8.00 (s, 1H), 7.27-7.19 (m, 1H), 7.19-7.11 (m, 1H), 6.45 (d, J=2.8 Hz, 1H), 5.42-5.18 (m, 1H), 4.28-4.06 (m, 4H), 3.94-3.73 (m, 3H), 3.22-3.01 (m, 3H), 2.92-2.82 (m, 1H), 2.25 (s, 3H), 2.22-1.93 (m, 8H), 1.91-1.75 (m, 3H), 1.73-1.60 (m, 1H); LCMS (ESI, M+1): m/z=735.4.

### Example 172

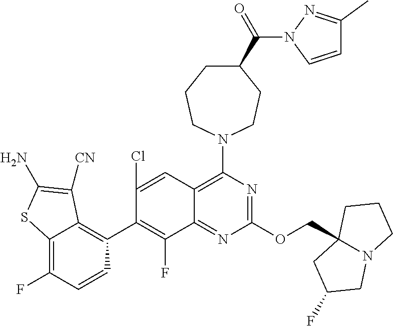

### 2-amino-4-((S)-6-chloro-8-fluoro-2-(((2R,7aS)-2-fluorotetrahydro-1H-pyrrolizin-7a(5H)-yl)methoxy)-4-((R)-4-(3-methyl-1H-pyrazole-1-carbonyl) azepan-1-yl)quinazolin-7-yl)-7-fluorobenzo[b]thiophene-3-carbonitrile

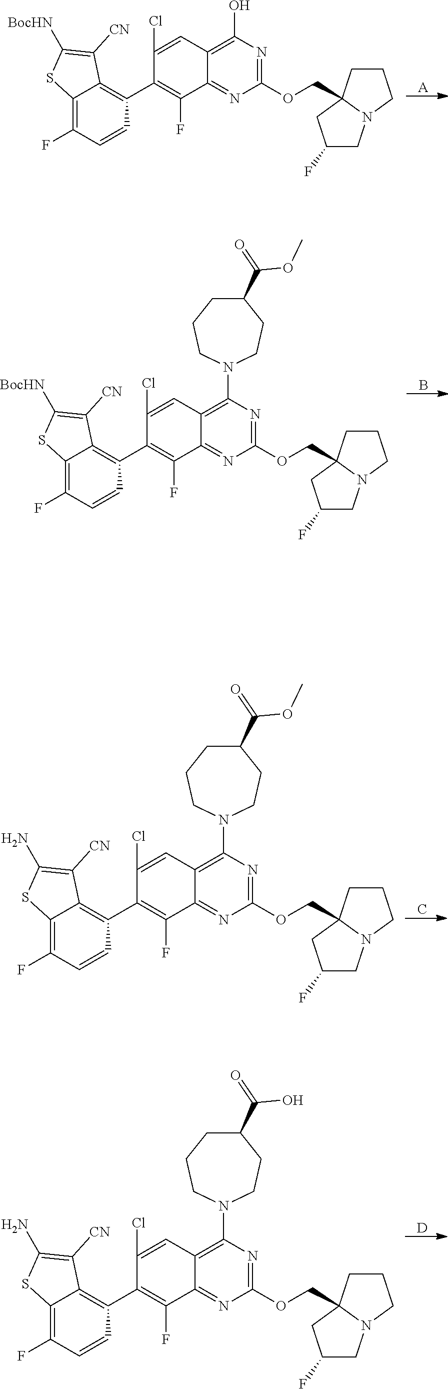

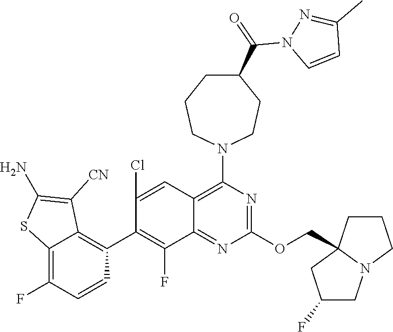

Step A. methyl (R)-1-((R)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution was added t-butyl(4-((R)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (700 mg, 1.00 equiv) and methyl (R)-azepane-4-carboxylate (511 mg, 3.0 equiv) in DMSO (7 mL) were added benzotriazol-1-yloxyl-tris-(pyrrolidino)-phosphonium hexafluorophosphate (2.82 g, 5.0 equiv) and TEA (329 mg, 3.0 equiv). The reaction was stirred at 30 °C for 48 hours. The mixture was filtered and purified with reversed phase flash chromatography [C18, 0.1% formic acid condition] to afford the title compound (270 mg 31% yield) as yellow solid; LCMS (ESI, M+1): m/z=785.3.

Step B. methyl (R)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl (R)-1-((R)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (270 mg, 1.0 equiv) in DCM (2 mL) was added TFA (4 mL). The reaction was stirred at 20 °C for 0.5 hours. The mixture was adjusted to pH>8 with saturated Na₂CO₃ aqueous (10 mL) at 0 °C and extracted with DCM (3×5 mL). The combined organic layers were dried over anhydrous sodium sulfate and concentrated to afford the title compound (220 mg, crude) as yellow solid; LCMS (ESI, M+1): m/z=685.2.

Step C. (R)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl (R)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (220 mg, 1.0 equiv) in MeOH (2.5 mL) and THF (1 mL) was added LiOH (23.1 mg, 3.0 equiv) in H₂O (0.5 mL). The reaction was stirred at 20 °C for 1 hour. The mixture was diluted with HCl (4 M, 5 mL) and purified with reversed phase flash chromatography [C18, 0.1% formic acid condition] to afford the title compound (150 mg, 68% yield) as yellow solid; LCMS (ESI, M+1): m/z=671.2.

Step D. 2-amino-4-((R)-6-chloro-8-fluoro-2-(((2R,7aS)-2-fluorotetrahydro-1H-pyrrolizin-7a(5H)-yl)methoxy)-4-((R)-4-(3-methyl-1H-pyrazole-1-carbonyl) azepan-1-yl)quinazolin-7-yl)-7-fluorobenzo[b]thiophene-3-carbonitrile: To a mixture of (R)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (130 mg, 1.0 equiv), 3-methyl-1H-pyrazole (47.7 mg, 3.0 equiv), TEA (58.8 mg, 3.0 equiv) in DMAC (1 mL) were added HOBt (39.3 mg, 1.5 equiv) and EDCI (55.7 mg, 1.5 equiv). The reaction was stirred at 20 °C for 12 hours. The mixture was filtered and purified with reversed phase flash chromatography [C18, neutral condition] to afford the title compound (65.6 mg, 42% yield) as white solid; ¹H NMR (400 MHz, DMSO-d₆) δ=8.27 (d, J=2.8 Hz, 1H), 8.10 (s, 2H), 7.99 (s, 1H), 7.28-7.24 (m, 1H), 7.20-7.09 (m, 1H), 6.46 (d, J=2.8 Hz, 1H), 5.43-5.10 (m, 1H), 4.24-3.99 (m, 4H), 3.99-3.70 (m, 3H), 3.15-2.97 (m, 3H), 2.88-2.76 (m, 1H), 2.26 (s, 3H), 2.23-1.91 (m, 8H), 1.87-1.73 (m, 3H), 1.67-1.64 (m, 1H); LCMS (ESI, M+1): m/z=735.4.

### Example 173

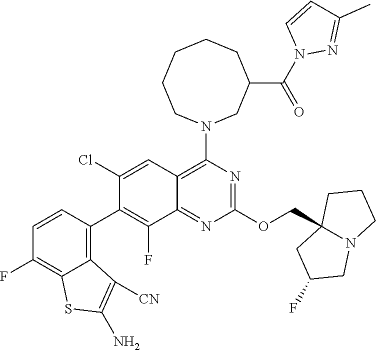 [SMILES: Cc1ccn(C(=O)C2CCCCCN(c3nc(OC[C@@]45CCCN4C[C@H](F)C5)nc4c(F)c(-c5ccc(F)c6sc(N)c(C#N)c56)c(Cl)cc34)C2)n1]

### 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl) azocan-1-yl)quinazolin-7-yl)-7-fluorobenzo[b]thiophene-3-carbonitrile

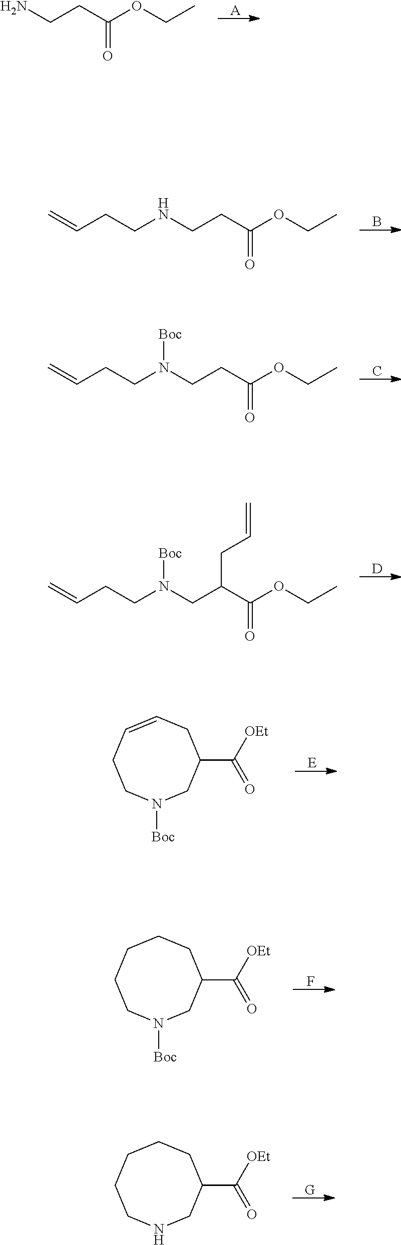

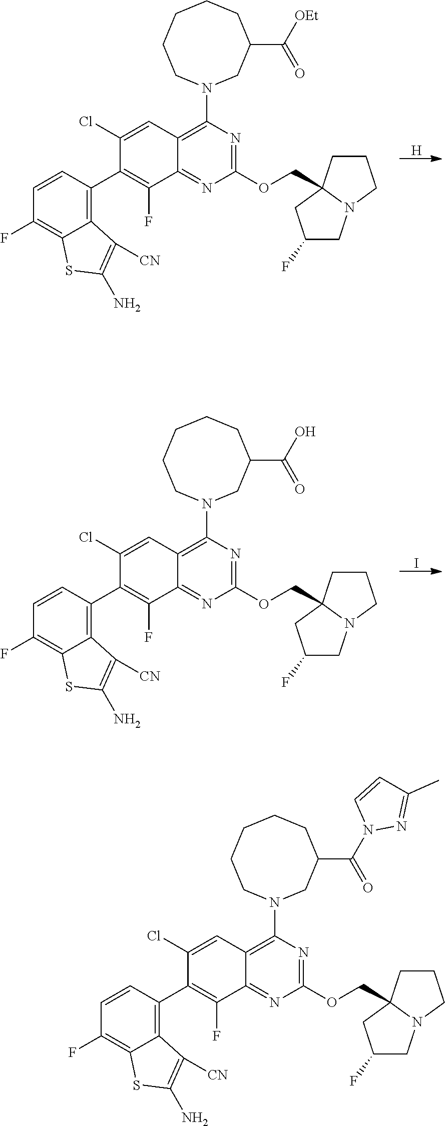

Step A. ethyl 3-(but-3-en-1-ylamino)propanoate: To a solution of ethyl ethyl 3-aminopropanoate (49.5 g, 1.5 equiv, HCl) in ACN (1000 mL) was added K₂CO₃ (87.5 g, 3.0 equiv). The reaction was stirred at 25 °C for 1 hour. 4-bromobut-1-ene (28.5 g, 1.0 equiv) was added. The reaction was stirred at 45 °C for 17 hours. The reaction was concentrated to afford the title compound (48.3 g, crude) as colorless oil; ¹H NMR (400 MHz, CHLOROFORM-d) δ=5.91-5.64 (m, 1H), 5.16-4.89 (m, 1H), 4.21-3.97 (m, 2H), 3.00-2.75 (m, 2H), 2.66 (t, J=6.8 Hz, 1H), 2.56-2.34 (m, 2H), 2.29-2.09 (m, 1H), 1.34-1.14 (m, 3H).

Step B. ethyl 3-(but-3-en-1-yl(tert-butoxycarbonyl)amino)propanoate: To a solution of ethyl 3-(but-3-enylamino)propanoate (43.3 g, 1.0 equiv) in DCM (450 mL) was added TEA (51.1 g, 2.0 equiv) and Boc₂O (82.7 g, 1.5 equiv). The reaction was stirred at 25 °C for 1 hour. The mixture was concentrated and purified by column chromatography (SiO₂, PE/EA=1/0 to 20:1) to afford the title compound (40.2 g, 59% yield) as yellow oil.

Step C. ethyl 2-((but-3-en-1-yl(tert-butoxycarbonyl)amino)methyl)pent-4-enoate: To a solution of ethyl 3-(but-3-en-1-yl(tert-butoxycarbonyl)amino)propanoate (20.0 g, 1.0 equiv) in THF (200 mL), the reaction was degassed and purged with nitrogen 3 times. LDA (2 M, 1.5 equiv) was added slowly at −65 °C, the reaction was stirred at −40 °C for 1 hour. 3-iodoprop-1-ene (13.6 g, 1.1 equiv) was added at −65 °C. The reaction was stirred at −40 °C for 0.5 hours. The mixture was quenched with water (100 mL) at 0 °C and extracted with ethyl acetate (3×100 mL). The combined organic layer was dried over anhydrous Na₂SO₄, filtered, concentrated and purified by column chromatography (SiO₂, PE/EA=1/0 to 20:1) to afford the title compound (3.90 g, 15% yield) as yellow oil; ¹H NMR (400 MHz, CHLOROFORM-d) δ=5.81-5.64 (m, 2H), 5.11-4.95 (m, 4H), 4.16-4.06 (m, 2H), 3.50-3.21 (m, 3H), 3.17-3.05 (m, 1H), 2.95-2.68 (m, 1H), 2.39-2.11 (m, 4H), 1.44 (s, 9H), 1.23 (t, J=7.2 Hz, 3H).

Step D. 1-(tert-butyl) 3-ethyl(Z)-3,4,7,8-tetrahydroazocine-1,3 (2H)-dicarboxylate: To a solution of ethyl 2-((but-3-en-1-yl(tert-butoxycarbonyl)amino)methyl)pent-4-enoate (1.00 g, 1.0 equiv) in DCM (150 mL) was added Grubbs catalyst (265 mg, 0.10 equiv). The reaction was purged with nitrogen 3 times. The reaction was stirred at 40 °C for 1.5 hours. The mixture was filtered, concentrated and purified by column chromatography (SiO₂, PE/EA=1/0 to 20/1) to afford the title compound (650 mg, 71% yield) as brown oil; ¹H NMR (400 MHz, CHLOROFORM-d) δ=5.96-5.59 (m, 2H), 4.20-4.06 (m, 3H), 3.91-3.75 (m, 1H), 3.42-3.05 (m, 1H), 3.01-2.89 (m, 1H), 2.83-2.58 (m, 1H), 2.49-2.21 (m, 3H), 2.19-2.06 (m, 1H), 1.47 (d, J=3.2 Hz, 9H), 1.26 (td, J=7.2, 10.3 Hz, 3H).

Step E. 1-(tert-butyl) 3-ethyl azocane-1,3-dicarboxylate: To a mixture of Pd/C (200 mg, 10% purity) in EtOH (20 mL) was added 1-(tert-butyl) 3-ethyl(Z)-3,4,7,8-tetrahydroazocine-1,3 (2H)-dicarboxylate (650 mg, 1.0 equiv). The suspension was degassed under vacuum and purged with H₂ several times. The reaction was stirred under H₂ (15 psi) at 25 °C for 1 hour. The mixture was filtered and concentrated to afford the title compound (381 mg, crude) as brown oil; ¹H NMR (400 MHz, CHLOROFORM-d) δ=4.20-4.05 (m, 2H), 3.94-3.60 (m, 2H), 3.16 (ddd, J=6.8, 11.2, 14.4 Hz, 1H), 3.03-2.80 (m, 2H), 1.98-1.82 (m, 1H), 1.77-1.59 (m, 5H), 1.49-1.42 (m, 10H), 1.29-1.20 (m, 4H).

Step F. ethyl azocane-3-carboxylate: A solution of 1-(tert-butyl) 3-ethyl azocane-1,3-dicarboxylate (381 mg, 1.0 equiv) in HCl·MeOH (2 M, 6.7 equiv) was stirred at 25 °C for 0.5 hours. The mixture was concentrated, diluted with MeOH (3 mL), adjusted to pH about 8 using NaHCO₃solid, filtered and concentrated to afford the title compound (236 mg, crude) as yellow oil; ¹H NMR (400 MHz, CHLOROFORM-d) δ=4.18-3.99 (m, 2H), 3.31-3.22 (m, 1H), 3.19-3.13 (m, 2H), 3.02-2.89 (m, 2H), 2.17-2.06 (m, 1H), 1.96-1.81 (m, 4H), 1.68 (br s, 3H), 1.20 (t, J=7.2 Hz, 3H).

Step G. ethyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-3-carboxylate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (300 mg, 1.0 equiv) in DMF (4 mL) was added DIEA (355 mg, 5.0 equiv) and PyBOP (343 mg, 1.2 equiv). The reaction was stirred at 25 °C for 0.5 hours. Ethyl azocane-3-carboxylate (203 mg, 2.0 equiv) was added into the mixture. The reaction was stirred at 25 °C for 1 hour. The mixture was filtered and purified by prep-HPLC [Phenomenex luna C18 150×25 mm×10 μm; A: water (FA), B %: ACN, B %: 28%-58% over 9 min] to afford the title compound (250 mg, 62% yield, HCOOH) as yellow oil; LCMS (ESI, M+1): m/z=713.4.

Step H. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-3-carboxylic acid: A solution of ethyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-3-carboxylate (100 mg, 1.0 equiv, FA) in MeOH (1.5 mL) was added LiOH·H₂O (2 M, 1.5 mL, 21 equiv). The reaction was stirred at 40 °C for 0.5 hours. The mixture was filtered and purified by reversed-phase flash (0.1% FA condition) to afford the title compound (35.0 mg, 34% yield HCOOH) as white solid; LCMS (ESI, M+1): m/z=685.2.

Step I. 2-amino-4-(6-chloro-8-fluoro-2-(((2R,7aS)-2-fluorotetrahydro-1H-pyrrolizin-7a(5H)-yl)methoxy)-4-(3-(3-methyl-1H-pyrazole-1-carbonyl) azocan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azocane-3-carboxylic acid (25.0 mg, 1.0 equiv, HCOOH) and 3-methyl-1H-pyrazole (8.42 mg, 3.0 equiv) in DMF (0.5 mL) were added DIEA (22.1 mg, 5.0 equiv) and HATU (14.3 mg, 1.1 equiv). The reaction was stirred at 25 °C for 1 hour. The mixture was filtered and purified by prep-HPLC [Waters Xbridge 150×25 mm×5 μm; A: water (NH₄HCO₃), B: ACN, B %: 60%-90% over 15 min] to afford the title compound (9.57 mg, 35% yield) as yellow solid; ¹H NMR (400 MHz, CHLOROFORM-d) δ=1.84-1.94 (m, 6H), 2.06-2.27 (m, 8H), 2.32 (dd, J=5.32, 2.44 Hz, 3H), 2.90-3.02 (m, 1H), 3.11-3.22 (m, 1H), 3.25-3.37 (m, 1H), 3.55-4.02 (m, 3H), 4.06-4.24 (m, 2H), 4.36-4.56 (m, 2H), 4.59-4.85 (m, 1H), 5.07-5.41 (m, 1H), 5.48-5.74 (m, 2H), 6.27 (d, J=2.76 Hz, 1H), 6.96-7.07 (m, 1H), 7.18-7.23 (m, 1H), 7.91 (s, 1H), 8.15-8.27 (m, 1H); LCMS (ESI, M+1): m/z=749.3.

### Example 174

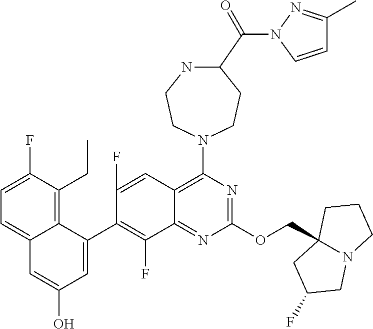 [SMILES: CCc1c(F)ccc2cc(O)cc(-c3c(F)cc4c(N5CCNC(C(=O)n6ccc(C)n6)CC5)nc(OC[C@@]56CCCN5C[C@H](F)C6)nc4c3F)c12]

### (1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl) azepan-4-yl)(3-methyl-1H-pyrazol-1-yl)methanone

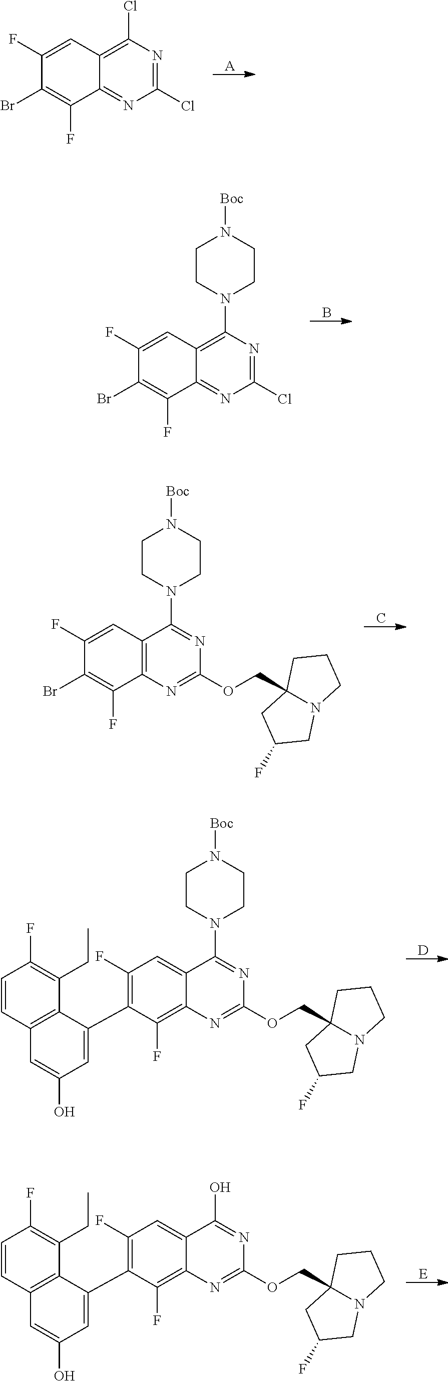

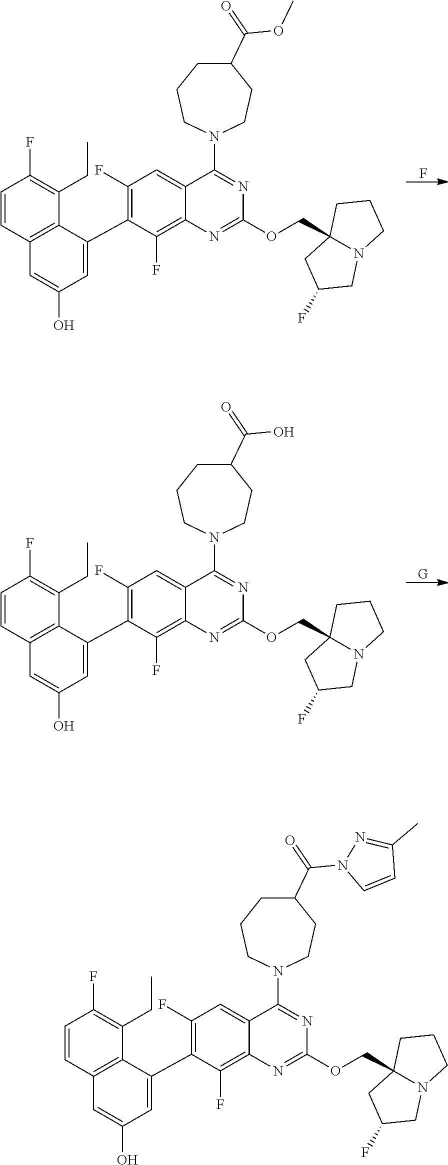

Step A. tert-butyl 4-(7-bromo-2-chloro-6,8-difluoroquinazolin-4-yl)piperazine-1-carboxylate: To a solution of 7-bromo-2,4-dichloro-6,8-difluoroquinazoline (5.00 g, 1.0 equiv) and DIEA (10.3 g, 5.0 equiv) in DCM (50 mL) was added tert-butyl piperazine-1-carboxylate (3.86 g, 1.3 equiv) at −40 °C. The reaction was stirred at −40 °C for 1 hour. The mixture was diluted with water (50 mL) and extracted with DCM (3×80 mL). The combined organic layers were dried over anhydrous sodium sulfate, concentrated and triturated with ACN at 25 °C for 0.5 hours to afford the title compound. (7.00 g, 93% yield) as white solid; LCMS (ESI, M+1, M+3): m/z=463.1, 465.1.

Step B. tert-butyl 4-(7-bromo-6,8-difluoro-2-(((2R,7aS)-2-fluorotetrahydro-1H-pyrrolizin-7a(5H)-yl)methoxy)quinazolin-4-yl)piperazine-1-carboxylate: To a solution of tert-butyl 4-(7-bromo-2-chloro-6,8-difluoroquinazolin-4-yl)piperazine-1-carboxylate (5.20 g, 1.0 equiv), ((2R,7aS)-2-fluorotetrahydro-1H-pyrrolizin-7a(5H)-yl)methanol (2.68 g, 1.5 equiv) and 4 Å molecular sieve (500 mg) in THF (25 mL) and DMF (25 mL) were added Cs₂CO₃ (11.0 g, 3.0 equiv) and DABCO (1.26 g, 1.0 equiv). The reaction was stirred at 25 °C for 4 hours. The mixture was diluted with water (60 mL) and extracted with ethyl acetate (3×30 mL). The combined organic layers were washed with brine (3×90 mL), dried over anhydrous sodium sulfate, concentrated and triturated with ACN at 25 °C for 0.5 hours to afford the title compound. (5.50 g, 81% yield) as white solid; LCMS (ESI, M+1, M+3): m/z=586.2, 588.2.

Step C. tert-butyl 4-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)piperazine-1-carboxylate: To a mixture of tert-butyl 4-(7-bromo-6,8-difluoro-2-(((2R,7aS)-2-fluorotetrahydro-1H-pyrrolizin-7a(5H)-yl)methoxy)quinazolin-4-yl)piperazine-1-carboxylate (1.8 g, 1.0 equiv) and 5-ethyl-6-fluoro-4-(4,4,5,5-tetramethyl-1,3,2-dioxaborolan-2-yl)naphthalen-2-ol (1.46 g, 1.5 equiv) in methoxycyclopentane (18 mL) and H₂O (6 mL) were added CataCXium A Pd G3 (223.53 mg, 0.1 equiv) and K₃PO₄ (1.95 g, 3.0 equiv). The reaction was degassed and purged with nitrogen 3 times. The reaction was stirred at 90 °C for 2 hours. The mixture was diluted with water (40 mL) and extracted with ethyl acetate (3×40 mL). The combined organic layers were dried over anhydrous sodium sulfate, concentrated and purified by reversed phase flash [C18, 0.1% formic acid condition] to afford the title compound. (1.97 g, 90% yield) as yellow solid; LCMS (ESI, M+1): m/z=696.4.

Step D. 7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-ol: To a solution of tert-butyl 4-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)piperazine-1-carboxylate (5.9 g, 1.0 equiv) in EtOH (60 mL) and H₂O (85 mL) was added NaOH (3.39 g, 10 equiv). The reaction was stirred at 50 °C for 24 hours. The reaction mixture was concentrated. The mixture was adjusted pH to 8 with HCl (1M, 70 mL) and extracted with ethyl acetate (3×30 mL). The combined organic layers were dried over anhydrous sodium sulfate, concentrated and purified by reversed phase flash [C18, 0.1% formic acid condition] to afford the title compound. (3.6 g, 80% yield) as yellow solid; LCMS (ESI, M+1): m/z=528.3.

Step E. methyl 1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)azepane-4-carboxylate: To a solution of 7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-ol (300 mg, 1.0 equiv) and (Benzotriazol-1-yloxy)tripyrrolidinophosphonium hexafluorophosphate (592 mg, 2.0 equiv) in DMSO (3 mL) was added TEA (288 mg, 5.0 equiv). The reaction was stirred at 30 °C for 0.5 hours. And then methyl azepane-4-carboxylate (330 mg, 3.0 equiv, HCl) was added to above mixture. The reaction was stirred at 30 °C for 12 hours. The residue was filtered, washed with DMSO (1 mL) and purified by reversed phase flash [C18, 0.1% formic acid condition] to afford the title compound. (210 mg, 54% yield) as yellow solid; LCMS (ESI, M+1): m/z=667.3.

Step F. 1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)azepane-4-carboxylic acid: To a solution of methyl 1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)azepane-4-carboxylate (210 mg, 1.0 equiv) in THF (0.6 mL), MeOH (0.4 mL) and H₂O (0.6 mL) was added LiOH·H₂O (39.6 mg, 3.0 equiv) at 0 °C. The reaction was stirred at 20 °C for 2 hours. The residue was filtered, washed with ACN (1 mL) and purified by reversed phase flash [C18, 0.1% formic acid condition] to afford the title compound. (185 mg, 89% yield) as yellow solid; LCMS (ESI, M+1): m/z=653.3.

Step G (1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl) azepan-4-yl) (3-methyl-1H-pyrazol-1-yl)methanone: To a solution of 1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)azepane-4-carboxylic acid (85 mg, 1.0 equiv), EDCI (37.4 mg, 1.5 equiv) and HOBt (26.4 mg, 1.5 equiv) in DMF (1 mL) were added DIEA (50.5 mg, 3.0 equiv) and 3-methyl-1H-pyrazole (21.4 mg, 2.0 equiv). The reaction was stirred at 20 °C for 12 hours. The mixture was filtered and purified with reversed phase flash [C18, H₂O condition] to afford the title compound (25.7 mg, 27% yield) as white solid; ¹H NMR (400 MHz, DMSO-d₆) δ=9.99 (br s, 1H), 8.28 (br s, 1H), 7.89-7.70 (m, 2H), 7.46-7.28 (m, 2H), 7.00 (br s, 1H), 6.46 (d, J=2.4 Hz, 1H), 5.39-5.15 (m, 1H), 4.26-4.16 (m, 2H), 4.12-4.06 (m, 1H), 4.05-3.75 (m, 4H), 3.10-2.97 (m, 3H), 2.85-2.78 (m, 1H), 2.38-2.23 (m, 5H), 2.22-1.93 (m, 8H), 1.88-1.65 (m, 4H), 0.76 (td, J=7.2, 14.4 Hz, 3H); LCMS (ESI, M+1): m/z=717.3.

### Example 175

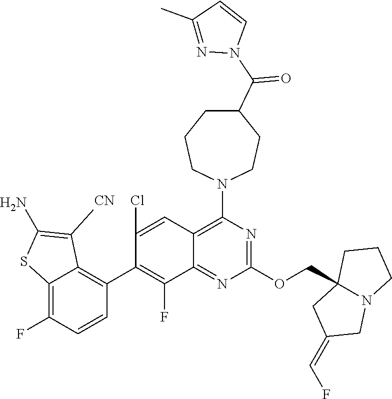

### 2-amino-4-(6-chloro-8-fluoro-2-(((S,Z)-2-(fluoromethylene)tetrahydro-1H-pyrrolizin-7a(5H)-yl)methoxy)-4-(4-(3-methyl-1H-pyrazole-1-carbonyl) azepan-1-yl)quinazolin-7-yl)-7-fluorobenzo[b]thiophene-3-carbonitrile

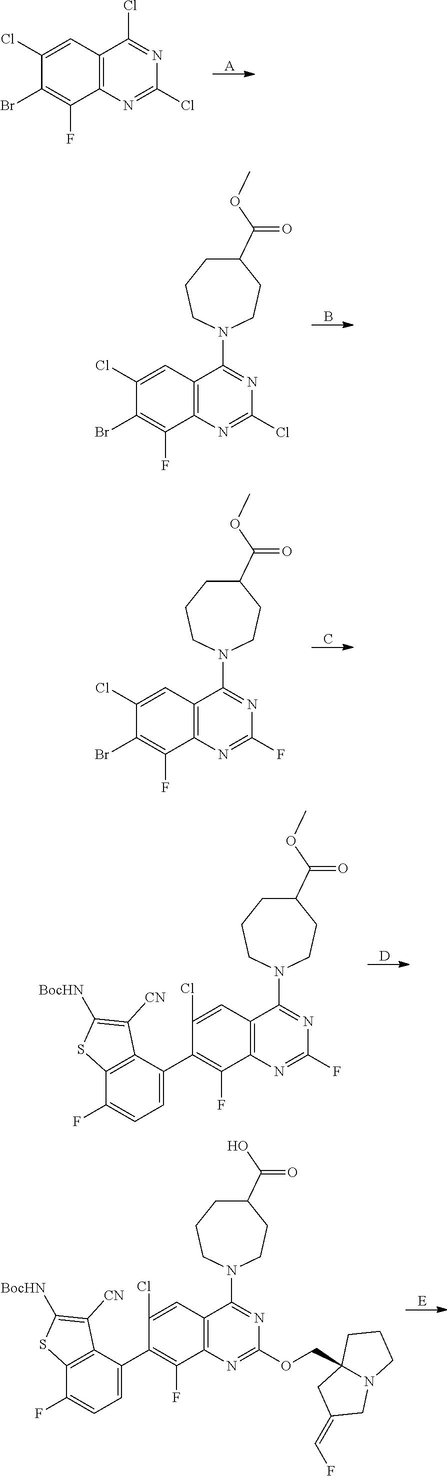

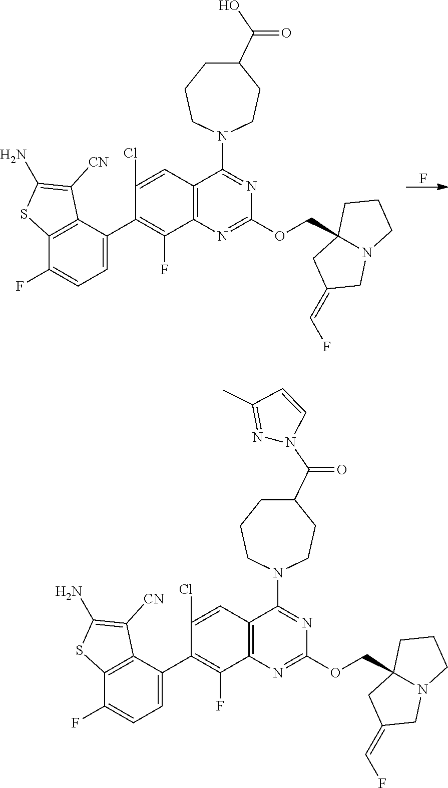

Step A. methyl 1-(7-bromo-2,6-dichloro-8-fluoroquinazolin-4-yl)azepane-4-carboxylate: To a solution of 7-bromo-2,4,6-trichloro-8-fluoroquinazoline (3.00 g, 1.0 equiv) and DIEA (7.04 g, 6.0 equiv) in DCM (30 mL) was added methyl azepane-4-carboxylate hydrogen chloride (1.76 g, 1.0 equiv) at −40 °C. The reaction was stirred at −40 °C for 2 hours. The mixture was diluted with water (150 mL) and extracted with DCM (4×35 mL). The combined organic layers were washed with brine (70 mL), dried over anhydrous sodium sulfate, concentrated and triturated with acetonitrile (20 mL) to afford the title compound (3.11 g, 74% yield) as yellow solid; LCMS (ESI, M+1, M+3): m/z=450.1, 452.1.

Step B. methyl 1-(7-bromo-6-chloro-2,8-difluoroquinazolin-4-yl)azepane-4-carboxylate: To a solution of methyl 1-(7-bromo-2,6-dichloro-8-fluoroquinazolin-4-yl)azepane-4-carboxylate (3.01 g, 1.0 equiv) and 18-crown-6 ether (176 mg, 0.1 equiv) in DMSO (30 mL) was added KF (3.88 g, 10.0 equiv). The reaction was stirred at 130 °C for 2 hours. The mixture was diluted with water (150 mL) and extracted with ethyl acetate (4×35 mL). The combined organic layers were washed with brine (2×70 mL), dried over anhydrous sodium sulfate, concentrated and triturated with MeCN to afford the title compound (2.85 g, 87% yield) as yellow solid; LCMS (ESI, M+1, M+3): m/z=434.1, 436.1.

Step C. methyl 1-(7-(2-((tert-butoxycarbonyl)amino)-3-cyano-7-fluorobenzo[b]thiophen-4-yl)-6-chloro-2,8-difluoroquinazolin-4-yl)azepane-4-carboxylate: To a mixture of methyl 1-(7-bromo-6-chloro-2,8-difluoroquinazolin-4-yl)azepane-4-carboxylate (2.60 g, 1.0 equiv) and tert-butyl(3-cyano-4-(5,5-dimethyl-1,3,2-dioxaborinan-2-yl)-7-fluorobenzo[b]thiophen-2-yl)carbamate (2.90 g, 1.2 equiv) in THF (30 mL) were added DPEPhosPdCl₂ (411 mg, 0.1 equiv) and Cs₂CO₃ (5.84 g, 3.0 equiv). The reaction was degassed and purged with nitrogen 3 times. The reaction was stirred at 60 °C for 6 hours. The mixture was diluted with water (50 mL) and extracted with EtOAc (4×25 mL). The combined organic layers were washed with brine (40 mL), dried over anhydrous sodium sulfate, concentrated and purified with reversed phase flash [water (0.1% formic acid)/acetonitrile] to afford the title compound (1.47 g, 38% yield) as brown solid; LCMS (ESI, M+1): m/z=646.2.

Step D. 1-(7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)azepane-4-carboxylic acid: To a solution of (S,Z)-(2-(fluoromethylene)tetrahydro-1H-pyrrolizin-7a(5H)-yl)methanol (79.5 mg, 1.0 equiv) in THF (3 mL) was added NaH (46.4 mg, 60% purity, 2.5 equiv) at 0 °C. The reaction was stirred at 0 °C for 0.2 hours. Methyl 1-(7-(2-((tert-butoxycarbonyl)amino)-3-cyano-7-fluorobenzo[b]thiophen-4-yl)-6-chloro-2,8-difluoroquinazolin-4-yl)azepane-4-carboxylate (300 mg, 1.0 equiv) was added into above mixture. The reaction was stirred at 0 °C for 2 hours. The mixture quenched with saturated NH₄Cl aqueous (2 mL) and water (8 mL). The mixture was extracted with EtOAc (4×8 mL). The combined organic layers were washed with brine (10 mL), dried over anhydrous sodium sulfate and concentrated to afford the title compound (365 mg, 96% yield) as yellow solid; LCMS (ESI, M+1): m/z=783.3.

Step E. 1-(7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)azepane-4-carboxylic acid: To a solution of 1-(7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)azepane-4-carboxylic acid (365 mg, 1.0 equiv) in DCM (3 mL) was added TFA (5.93 g, 111.5 equiv) at 0 °C. The reaction was stirred at 20 °C for 1 hour. The mixture was concentrated. The residue was diluted with DCM (10 mL) and saturated NaHCO₃ aqueous (10 mL) at 0 °C. The mixture was extracted with DCM (4×10 mL). The combined organic layers were washed brine (15 mL), dried over anhydrous sodium sulfate, concentrated and purified with reversed phase flash [water (0.1% formic acid)/acetonitrile=7/3] to afford the title compound (168 mg, 50% yield) as white solid; LCMS (ESI, M+1): m/z=683.2.

Step F. 2-amino-4-(6-chloro-8-fluoro-2-(((S,Z)-2-(fluoromethylene)tetrahydro-1H-pyrrolizin-7a(5H)-yl)methoxy)-4-(4-(3-methyl-1H-pyrazole-1-carbonyl) azepan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)azepane-4-carboxylic acid (100 mg, 1.0 eq) and 3-methyl-1H-pyrazole (30.1 mg, 2.5 equiv) in DMAc (1 mL) were added TEA (66.7 mg, 4.5 equiv), EDCI (50.5 mg, 1.8 equiv) and HOBt (37.6 mg, 1.9 equiv). The reaction was stirred at 25 °C for 14 hours. The mixture was purified with prep-HPLC [column: Waters Xbridge 150×25 mm×10 μm; mobile phase: water (NH₄HCO₃)-acetonitrile; gradient: 72%-92% B over 8 minutes] to afford the title compound (32.5 mg, 28% yield) as off-white solid; ¹H NMR (400 MHz, DMSO-d₆) δ=8.27 (d, J=2.4 Hz, 1H), 8.11 (s, 2H), 7.99 (s, 1H), 7.25 (ddd, J=5.2, 8.0, 13.2 Hz, 1H), 7.19-7.10 (m, 1H), 6.87-6.59 (m, 1H), 6.45 (d, J=2.8 Hz, 1H), 4.28-4.11 (m, 2H), 4.11-3.98 (m, 2H), 3.97-3.66 (m, 4H), 3.30-3.22 (m, 1H), 3.05-2.95 (m, 1H), 2.61-2.52 (m, 2H), 2.37-2.19 (m, 5H), 2.18-1.90 (m, 5H), 1.88-1.59 (m, 4H); LCMS (ESI, M+1): m/z=747.3.

### Example 176

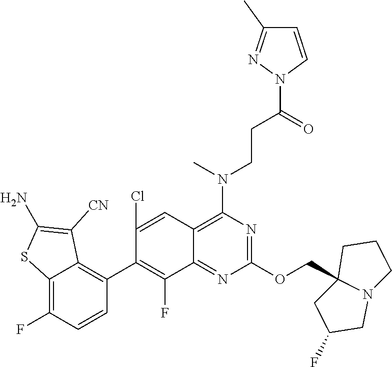 [SMILES: Cc1ccn(C(=O)CCN(C)c2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-(methyl(3-(3-methyl-1H-pyrazol-1-yl)-3-oxopropyl)amino)quinazolin-7-yl)-7-fluorobenzo[b]thiophene-3-carbonitrile

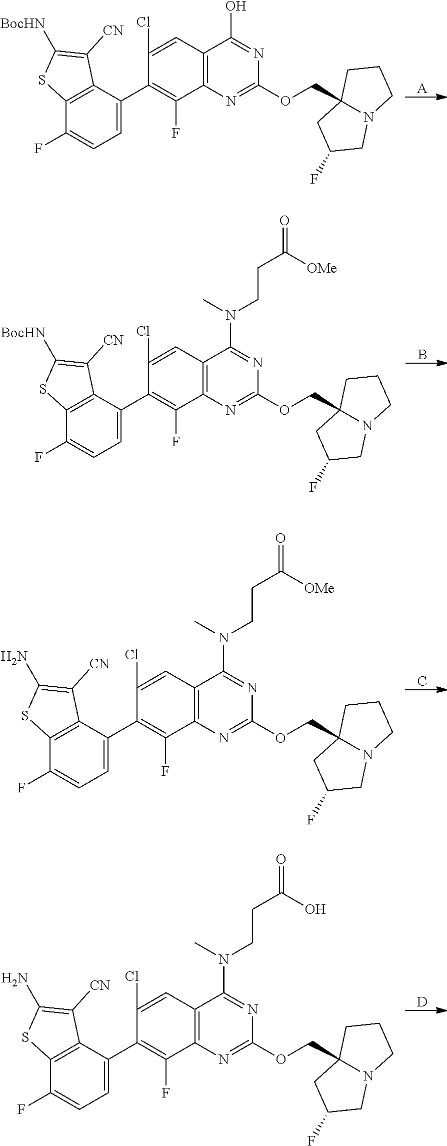

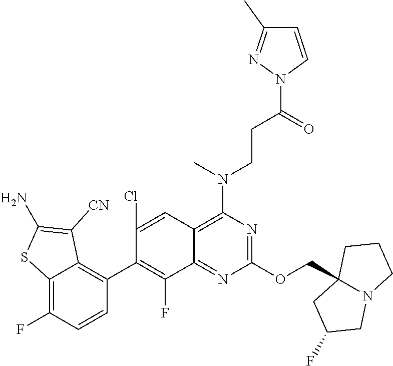 [SMILES: Cc1ccn(C(=O)CCN(C)c2nc(OC[C@@]34CCCN3C[C@H](F)C4)nc3c(F)c(-c4ccc(F)c5sc(N)c(C#N)c45)c(Cl)cc23)n1]

Step A. methyl 3-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)propanoate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv) in DMF (5 mL) were added DIEA (300 mg, 3.0 equiv), PyBOP (886 mg, 2.2 equiv) and methyl 3-(methylamino)propanoate (136 mg, 1.5 equiv). The reaction was stirred at 25 °C for 1 hour. The mixture was diluted with water (25 mL) and extracted with ethyl acetate (20 mL×3). The combined organic layers were washed with brine (30 mL), dried over anhydrous sodium sulfate and concentrated to afford the title compound (570 mg, crude) as yellow oil; LCMS (ESI, M+1): m/z=745.2.

Step B. methyl 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)propanoate: To a solution of methyl 3-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)propanoate (470 mg, 1.0 equiv) in DCM (15 mL) was added TFA (5 mL). The reaction was stirred at 25 °C for 1 hour. The mixture was concentrated, dissolved in MeOH (3 mL) and adjusted to pH=7 with NaHCO₃solid, filtered and concentrated to afford the title compound (400 mg, crude) as yellow oil; LCMS (ESI, M+1): m/z=645.2.

Step C. 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino) propanoic acid: To a solution of methyl 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)propanoate (400 mg, 1.0 equiv) in MeOH (3 mL) was added LiOH·H₂O (1.0 mL, 3.2 equiv, 2M). The reaction was stirred at 25 °C for 3 hours. The mixture was filtered and purified with prep-HPLC [column: Phenomenex luna C18 150×40 mm×15 μm; mobile phase: [water (FA)-ACN]; gradient: 18%-48% B over 15 min] to afford the title compound (198 mg, 97% yield) as white solid; LCMS (ESI, M+1): m/z=631.1.

Step D. 2-amino-4-(6-chloro-8-fluoro-2-(((2R,7aS)-2-fluorotetrahydro-1H-pyrrolizin-7a(5H)-yl)methoxy)-4-(methyl(3-(3-methyl-1H-pyrazol-1-yl)-3-oxopropyl)amino)quinazolin-7-yl)-7-fluorobenzo[b]thiophene-3-carbonitrile: To a solution of 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino) propanoic acid (198 mg, 1.0 equiv) in DMF (1.5 mL) were added DIEA (203 mg, 5.0 equiv), 3-methyl-1H-pyrazole (38.6 mg, 1.5 equiv) and HATU (179 mg, 1.5 equiv). The reaction was stirred at 25 °C for 0.5 hours. The mixture was filtered and purified with prep-HPLC [column: Welch Xtimate C18 150×40 mm×10 μm; mobile phase: [water (NH₄HCO₃)-ACN]; gradient: 45%-75% B over 15 min] to afford the title compound (10.3 mg, 4.5% yield) as white solid; ¹H NMR (400 MHz, CHLOROFORM-d) δ=8.16 (d, J=2.8 Hz, 1H), 7.90 (d, J=1.6 Hz, 1H), 7.20 (dd, J=5.2, 8.4 Hz, 1H), 7.02 (t, J=9.2 Hz, 1H), 6.26 (d, J=2.8 Hz, 1H), 5.53 (br s, 2H), 5.33-5.20 (m, 1H), 4.31-4.21 (m, 2H), 4.15 (dd, J=6.8, 9.4 Hz, 2H), 3.64-3.59 (m, 2H), 3.54 (s, 3H), 3.27-3.21 (m, 2H), 3.15 (s, 1H), 2.98-2.93 (m, 1H), 2.30 (s, 3H), 2.28-2.11 (m, 4H), 1.93-1.88 (m, 2H); LCMS (ESI, M+1): m/z=695.3.

### Example 177

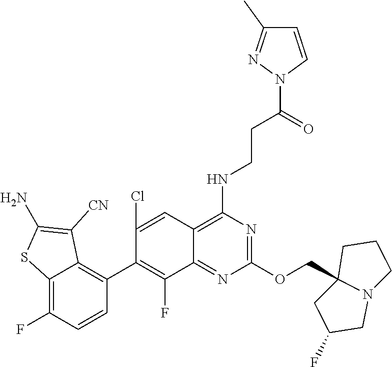 [SMILES: Cc1ccn(C(=O)CCNc2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-((3-(3-methyl-1H-pyrazol-1-yl)-3-oxopropyl)amino)quinazolin-7-yl)-7-fluorobenzo[b]thiophene-3-carbonitrile

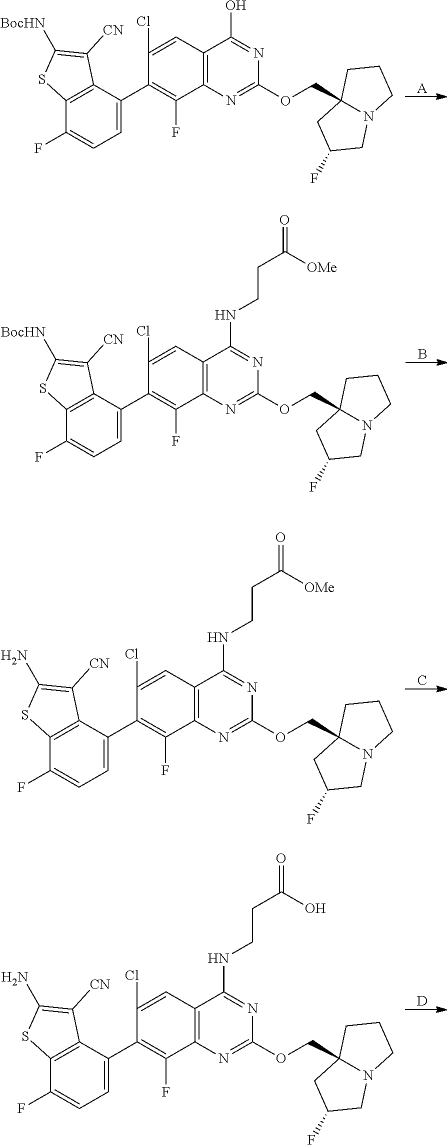

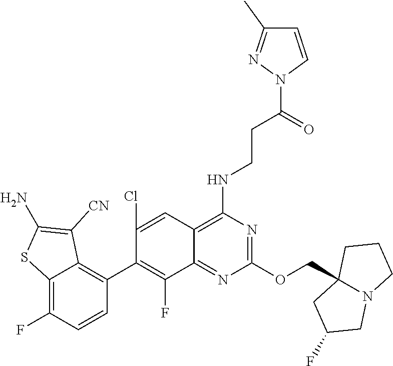 [SMILES: Cc1ccn(C(=O)CCNc2nc(OC[C@@]34CCCN3C[C@H](F)C4)nc3c(F)c(-c4ccc(F)c5sc(N)c(C#N)c45)c(Cl)cc23)n1]

Step A. methyl 3-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)propanoate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv) in DMF (5 mL) were added DIEA (300 mg, 3.0 equiv), PyBOP (886 mg, 2.2 equiv) and methyl 3-aminopropanoate (162 mg, 1.5 equiv). The reaction was stirred at 25 °C for 1 hour. The mixture was diluted with water (25 mL) and extracted with ethyl acetate (20 mL×3). The combined organic layers were washed with brine (30 mL), dried over anhydrous sodium sulfate and concentrated to afford the title compound (938 mg, crude) as yellow solid; LCMS (ESI, M+1): m/z=731.4.

Step B. methyl 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)propanoate: To a solution of methyl 3-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)propanoate (188 mg, 1.0 equiv) in DCM (1.8 mL) was added TFA (0.6 mL). The reaction was stirred at 25 °C for 1 hour. The mixture was concentrated, dissolved in MeOH (1 mL) and adjusted to pH=7 with NaHCO₃solid, filtered and concentrated to afford the title compound (228 mg, crude) as orange solid; LCMS (ESI, M+1): m/z=631.2.

Step C. 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino) propanoic acid: To a solution of methyl 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)propanoate (228 mg, 1.0 equiv) in MeOH (1.5 mL) was added LiOH·H₂O (0.5 mL, 2.8 equiv, 2M). The reaction was stirred at 25 °C for 0.5 hours. The mixture was filtered and purified with prep-HPLC [column: Phenomenex luna C18 150×40 mm×15 μm; mobile phase: [water (FA)-ACN]; gradient: 18%-48% B over 15 min] to afford the title compound (55.0 mg, 25% yield) as off-white solid; LCMS (ESI, M+1): m/z=617.3.

Step D. 2-amino-4-(6-chloro-8-fluoro-2-(((2R,7aS)-2-fluorotetrahydro-1H-pyrrolizin-7a(5H)-yl)methoxy)-4-((3-(3-methyl-1H-pyrazol-1-yl)-3-oxopropyl)amino)quinazolin-7-yl)-7-fluorobenzo[b]thiophene-3-carbonitrile: To a solution of 3-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino) propanoic acid (55.0 mg, 1.0 equiv) in DMF (0.5 mL) were added DIEA (57.6 mg, 5.0 equiv), 3-methyl-1H-pyrazole (11.0 mg, 1.5 equiv) and HATU (50.8 mg, 1.5 equiv). The reaction was stirred at 25 °C for 0.5 hours. The mixture was filtered and purified with prep-HPLC [column: Phenomenex luna C18 150×40 mm×10 μm; mobile phase: [water (FA)-ACN]; gradient: 22%-52% B over 15 min] to afford the title compound (9.82 mg, 15% yield) as off-white solid; ¹H NMR (400 MHz, CHLOROFORM-d) δ=8.19 (d, J=2.8 Hz, 1H), 7.55 (s, 1H), 7.17 (dd, J=5.2, 8.0 Hz, 1H), 7.01 (t, J=8.8 Hz, 1H), 6.95-6.89 (m, 1H), 6.30 (d, J=3.2 Hz, 1H), 5.60 (br s, 2H), 5.38-5.25 (m, 1H), 4.34-4.24 (m, 2H), 4.09-4.07 (m, 2H), 3.50 (br d, J=3.2 Hz, 2H), 3.39 (br d, J=1.6 Hz, 2H), 3.28-3.23 (m, 1H), 3.04-2.98 (m, 1H), 2.39 (s, 3H), 2.36-2.19 (m, 4H), 1.99 (br s, 2H); LCMS (ESI, M+1): m/z=681.3.

### Example 178

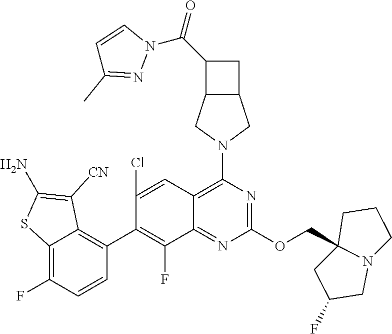 [SMILES: Cc1ccn(C(=O)C2CC3CN(c4nc(OC[C@@]56CCCN5C[C@H](F)C6)nc5c(F)c(-c6ccc(F)c7sc(N)c(C#N)c67)c(Cl)cc45)CC32)n1]

### 2-amino-4-(6-chloro-8-fluoro-2-(((2R,7aS)-2-fluorotetrahydro-1H-pyrrolizin-7a(5H)-yl)methoxy)-4-(6-(3-methyl-1H-pyrazole-1-carbonyl)-3-azabicyclo[3.2.0]heptan-3-yl)quinazolin-7-yl)-7-fluorobenzo[b]thiophene-3-carbonitrile

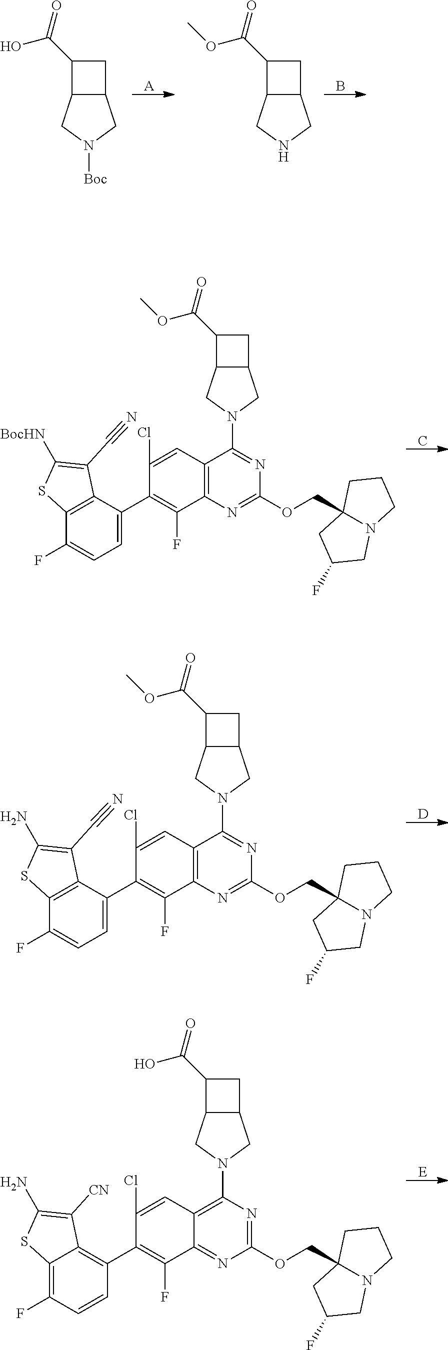

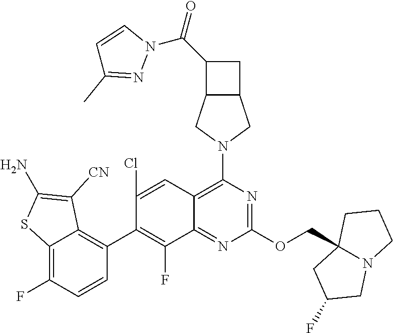 [SMILES: Cc1ccn(C(=O)C2CC3CN(c4nc(OC[C@@]56CCCN5C[C@H](F)C6)nc5c(F)c(-c6ccc(F)c7sc(N)c(C#N)c67)c(Cl)cc45)CC32)n1]

Step A. methyl 3-azabicyclo[3.2.0]heptane-6-carboxylate: A mixture of 3-(tert-butoxycarbonyl)-3-azabicyclo[3.2.0]heptane-6-carboxylic acid (500 mg, 1.0 equiv) in HCl·MeOH (2 M, 5 mL) was stirred at 0 °C for 2 hours. The mixture was concentrated to afford the title compound (500 mg, crude) as yellow oil.

Step B. methyl 3-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.0]heptane-6-carboxylate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv) and TEA (939 mg, 12 equiv) in DMSO (5 mL) were added methyl 3-azabicyclo[3.2.0]heptane-6-carboxylate (300 mg, crude) and benzotriazol-1-yloxyl-tris-(pyrrolidino)-phosphonium hexafluorophosphate (1.21 g, 3.0 equiv). The reaction was stirred at 25 °C for 2 hours. The mixture was filtered and purified with reversed phase flash [C18, 0.1% formic acid condition] to afford the title compound (450 mg, 73% yield) as yellow solid; LCMS (ESI, M+1): m/z=783.2.

Step C. methyl 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.0]heptane-6-carboxylate: To a solution of methyl 3-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.0]heptane-6-carboxylate (420 mg, 1.0 equiv) in DCM (4 mL) was added TFA (5 mL) at 0 °C. The reaction was stirred at 0 °C for 0.5 hours. The mixture was adjusted to pH=7 with saturated NaHCO₃ (15 mL) and extracted with DCM (2×20 mL). The combined organic layers were washed with brine (10 mL), dried over anhydrous sodium sulfate and concentrated to afford the title compound (450 mg, crude) as yellow solid.

Step D. 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.0]heptane-6-carboxylic acid: To a solution of methyl 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.0]heptane-6-carboxylate (440 mg, crude) in MeOH (4 mL) was added LiOH·H₂O (108 mg, 4.0 equiv). The reaction was stirred at 25 °C for 1 hour. The mixture was adjusted to pH=7 with saturated HCl (1 M) and purified with reversed phase flash [C18, 0.1% formic acid condition] to afford the title compound (130 mg, 26% yield) as yellow solid; LCMS (ESI, M+1): m/z=669.2.

Step E. 2-amino-4-(6-chloro-8-fluoro-2-(((2R,7aS)-2-fluorotetrahydro-1H-pyrrolizin-7a(5H)-yl)methoxy)-4-(6-(3-methyl-1H-pyrazole-1-carbonyl)-3-azabicyclo[3.2.0]heptan-3-yl)quinazolin-7-yl)-7-fluorobenzo[b]thiophene-3-carbonitrile: To a solution of 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.0]heptane-6-carboxylic acid (10.0 mg, 1.0 equiv) and 5-methyl-1H-pyrazole (12.3 mg, 10 equiv) in DMF (0.1 mL) were added EDCI (5.16 mg, 1.8 equiv), HOBt (4.04 mg, 2.0 equiv) and TEA (9.07 mg, 6.0 equiv). The reaction was stirred at 25 °C for 12 hours. The mixture was filtered and purified by prep-HPLC [column: Waters Xbridge 150×25 mm×5 μm; A: water (NH₄HCO₃), B: ACN; B %: 20%-40% over 10 min] and prep-HPLC [column: Waters Xbridge 150×25 mm×5 μm; A: water (NH₄HCO₃), B: ACN; B %: 58%-78% over 10 min] to afford the title compound (0.88 mg, 7.7% yield) as white solid; ¹H NMR (400 MHz, DMSO-d6) δ=8.32-8.20 (m, 2H), 8.10 (br s, 2H), 7.27-7.20 (m, 1H), 7.19-7.10 (m, 1H), 6.48-6.41 (m, 1H), 5.35-5.20 (m, 1H), 4.75-4.65 (m, 1H), 4.42-4.35 (m, 1H), 4.12 (br d, J=4.8 Hz, 2H), 4.04-3.99 (m, 3H), 3.06 (br d, J=13.6 Hz, 2H), 2.87-2.77 (m, 2H), 2.57 (br d, J=3.6 Hz, 1H), 2.28-2.19 (m, 3H), 2.16-1.98 (m, 5H), 1.91-1.70 (m, 4H); LCMS (ESI, M+1): m/z=733.4.

### Example 179

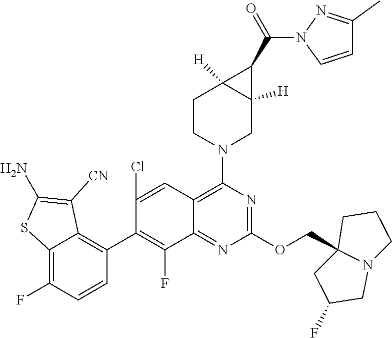 [SMILES: Cc1ccn(C(=O)[C@@H]2[C@@H]3CCN(c4nc(OC[C@@]56CCCN5C[C@H](F)C6)nc5c(F)c(-c6ccc(F)c7sc(N)c(C#N)c67)c(Cl)cc45)C[C@@H]32)n1]

### 2-amino-4-(6-chloro-8-fluoro-2-(((2R,7aS)-2-fluorotetrahydro-1H-pyrrolizin-7a(5H)-yl)methoxy)-4-((1S,6R)-7-(3-methyl-1H-pyrazole-1-carbonyl)-3-azabicyclo[4.1.0]heptan-3-yl)quinazolin-7-yl)-7-fluorobenzo[b]thiophene-3-carbonitrile

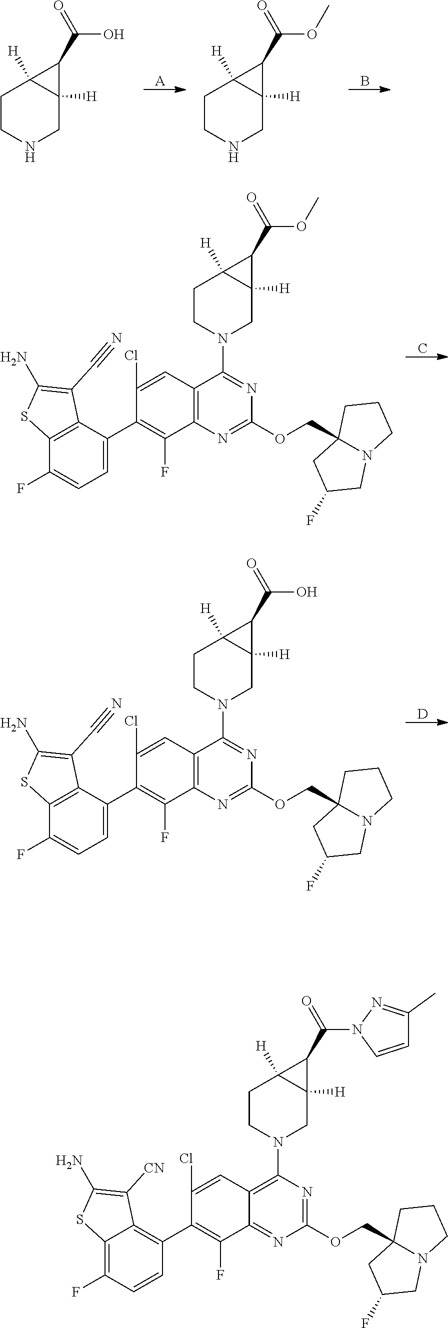

Step A. methyl(1S,6R,7R)-3-azabicyclo[4.1.0]heptane-7-carboxylate; A solution of (1S,6R,7R)-3-azabicyclo[4.1.0]heptane-7-carboxylic acid (500 mg, 1.0 equiv, HCl) in HCl·MeOH (2 M, 10.0 mL, 7.1 equiv) was stirred at 25 °C for 16 hours. The mixture was concentrated to afford the title compound (600 mg, crude, HCl) as yellow oil.

Step B. methyl(1S,6R,7R)-3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[4.1.0]heptane-7-carboxylate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (200 mg, 1.0 equiv) in DMF (2 mL) were added DIEA (284 mg, 6.0 equiv) and PyBOP (286 mg, 1.5 equiv). The reaction was stirred at 25 °C for 0.5 hours. methyl(1S,6R,7R)-3-azabicyclo[4.1.0]heptane-7-carboxylate (140 mg, 2.0 equiv, HCl) was added into the mixture. The reaction was stirred at 25 °C for 1.5 hours. The mixture was filtered and purified by prep-HPLC [Phenomenex luna C18 150×40 mm×15 μm; A: water (FA), B %: ACN, B %: 22%-52% over 15 min] to afford the title compound (170 mg, 64% yield, HCOOH) as yellow solid; LCMS (ESI, M+1): m/z=683.2.

Step C. (1S,6R,7R)-3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[4.1.0]heptane-7-carboxylic acid: To a solution of methyl(1S,6R,7R)-3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[4.1.0]heptane-7-carboxylate (200 mg, 1.0 equiv, HCOOH) in MeOH (2 mL) was added LiOH·H₂O (2 M, 2.00 mL, 15 equiv). The reaction was stirred at 40 °C for 1 hour. The mixture was filtered and purified by reversed-phase flash (0.1% FA condition) to afford the title compound (70.0 mg, 34% yield, HCOOH) as white solid; LCMS (ESI, M+1): m/z=669.2.

Step D. 2-amino-4-(6-chloro-8-fluoro-2-(((2R,7aS)-2-fluorotetrahydro-1H-pyrrolizin-7a(5H)-yl)methoxy)-4-((1S,6R,7R)-7-(3-methyl-1H-pyrazole-1-carbonyl)-3-azabicyclo[4.1.0]heptan-3-yl)quinazolin-7-yl)-7-fluorobenzo[b]thiophene-3-carbonitrile: To a solution of (1S,6R,7R)-3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[4.1.0]heptane-7-carboxylic acid (10.0 mg, 1.0 equiv, HCOOH), 3-methyl-1H-pyrazole (3.44 mg, 3.0 equiv) and TEA (8.49 mg, 6.0 equiv) in DMAc (0.2 mL) were added EDCI (4.02 mg, 1.5 equiv) and HOBt (2.83 mg, 1.5 equiv). The reaction was stirred at 25 °C for 14 hours. The mixture was filtered and purified by prep-HPLC [Waters Xbridge 150×25 mm×5 μm; A: water (NH₄HCO₃), B %: ACN, B %: 58%-88% over 9 min] to afford the title compound (5.26 mg, 47% yield) as yellow solid; ¹H NMR (400 MHz, CHLOROFORM-d) δ=8.26-8.08 (m, 1H), 7.73 (d, J=13.2 Hz, 1H), 7.22 (dt, J=5.2, 8.0 Hz, 1H), 7.07-7.00 (m, 1H), 6.28 (t, J=2.8 Hz, 1H), 5.57-5.42 (m, 2H), 5.38-5.19 (m, 1H), 4.40-4.19 (m, 3H), 4.05-3.83 (m, 2H), 3.48-3.17 (m, 5H), 3.05-2.96 (m, 1H), 2.34 (d, J=2.4 Hz, 3H), 2.30 (br s, 6H), 2.03-1.92 (m, 4H); LCMS (ESI, M+1): m/z=733.2.

### Example 180

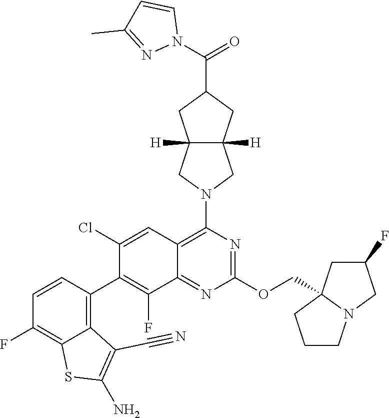 [SMILES: Cc1ccn(C(=O)C2C[C@@H]3CN(c4nc(OC[C@@]56CCCN5C[C@H](F)C6)nc5c(F)c(-c6ccc(F)c7sc(N)c(C#N)c67)c(Cl)cc45)C[C@@H]3C2)n1]

### 2-amino-4-(6-chloro-8-fluoro-2-(((2R,7aS)-2-fluorotetrahydro-1H-pyrrolizin-7a(5H)-yl)methoxy)-4-((3aR,6aS)-5-(3-methyl-1H-pyrazole-1-carbonyl)hexahydrocyclopenta[c]pyrrol-2 (1H)-yl)quinazolin-7-yl)-7-fluorobenzo[b]thiophene-3-carbonitrile

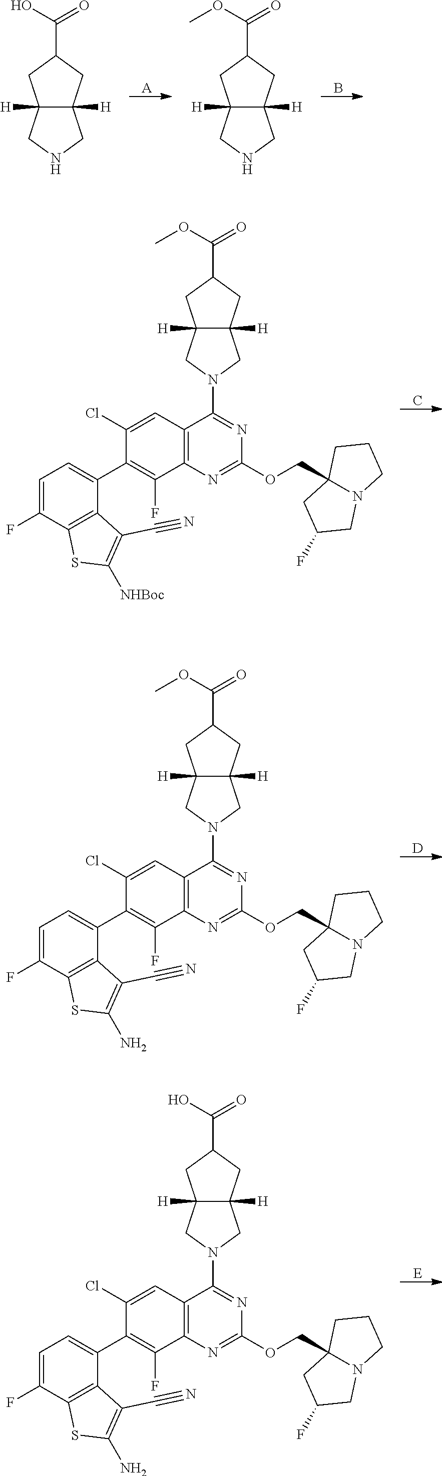

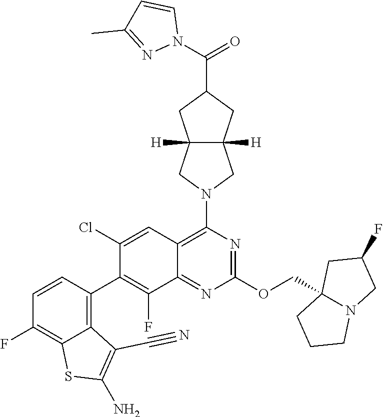 [SMILES: Cc1ccn(C(=O)C2C[C@@H]3CN(c4nc(OC[C@@]56CCCN5C[C@H](F)C6)nc5c(F)c(-c6ccc(F)c7sc(N)c(C#N)c67)c(Cl)cc45)C[C@@H]3C2)n1]

Step A. methyl(3aR,6aS)-octahydrocyclopenta[c]pyrrole-5-carboxylate: A mixture of (3aR,6aS)-octahydrocyclopenta[c]pyrrole-5-carboxylic acid (250 mg, 1.0 equiv) and HCl·MeOH (2 M, 2.50 mL) was stirred at 25 °C for 1 hour. The mixture was concentrated to afford the title compound (240 mg, crude, HCl salt) as yellow solid.

Step B. methyl(3aR,6aS)-2-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)octahydrocyclopenta[c]pyrrole-5-carboxylate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv) and methyl(3aR,6aS)-octahydrocyclopenta[c]pyrrole-5-carboxylate (239 mg, 1.5 equiv, HCl salt) in DMF (5 mL) were added PyBOP (604 mg, 1.5 equiv) and DIEA (1.00 g, 10 equiv). The reaction was stirred at 25 °C for 1 hour. The mixture was filtered and the filtrate was purified by reversed phase flash chromatography [C18, 0.1% formic acid condition] to afford the title compound (502 mg, 75% yield) as yellow solid; LCMS (ESI, M+1): m/z=797.2.

Step C. methyl(3aR,6aS)-2-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)octahydrocyclopenta[c]pyrrole-5-carboxylate: To a solution of methyl(3aR,6aS)-2-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)octahydrocyclopenta[c]pyrrole-5-carboxylate (502 mg, 1.0 equiv) in DCM (5 mL) was added TFA (5.02 mL, 107 equiv). The reaction was stirred at 25 °C for 1 hour. The mixture was concentrated, basified with saturated NaHCO₃ solution (20 mL) and extracted with ethyl acetate (3×20 mL). The combined organic layers were dried over anhydrous sodium sulfate, concentrated to afford the title compound (398 mg, crude) as yellow solid.

Step D. (3aR,6aS)-2-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)octahydrocyclopenta[c]pyrrole-5-carboxylic acid: To a solution of methyl(3aR,6aS)-2-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)octahydrocyclopenta[c]pyrrole-5-carboxylate (398 mg, 1.0 equiv) in MeOH (4 mL) was added LiOH·H₂O (2 M in H₂O, 4 mL, 14 equiv). The reaction was stirred at 25 °C for 0.5 hours. The mixture was filtered and the filtrate was purified by reversed phase flash chromatography [C18, 0.1% formic acid condition] to afford the title compound (256 mg, 65% yield) as white solid; LCMS (ESI, M+1): m/z=683.2.

Step E. 2-amino-4-(6-chloro-8-fluoro-2-(((2R,7aS)-2-fluorotetrahydro-1H-pyrrolizin-7a(5H)-yl)methoxy)-4-((3aR,6aS)-5-(3-methyl-1H-pyrazole-1-carbonyl)hexahydrocyclopenta[c]pyrrol-2 (1H)-yl)quinazolin-7-yl)-7-fluorobenzo[b]thiophene-3-carbonitrile: To a solution of (3aR,6aS)-2-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)octahydrocyclopenta[c]pyrrole-5-carboxylic acid (80 mg, 1.0 equiv) and 3-methyl-1H-pyrazole (48.1 mg, 5 equiv) in DMF (0.9 mL) were added HATU (89 mg, 2.0 equiv) and DIEA (75.7 mg, 5.0 equiv). The reaction was stirred at 25 °C for 0.5 hour. The mixture was filtered and purified by prep-HPLC [Waters Xbridge 150× 25 mm×5 μm; A: water (NH₄HCO₃), B: ACN, B %: 58%-88% over 9 min] to afford the title compound (22.2 mg, 24.8% yield) as yellow solid; ¹H NMR (400 MHz, CHLOROFORM-d) δ=8.17 (d, J=2.8 Hz, 1H), 8.05 (d, J=1.6 Hz, 1H), 7.21 (dd, J=4.8, 8.0 Hz, 1H), 7.08-6.96 (m, 1H), 6.28 (d, J=2.8 Hz, 1H), 5.48 (br s, 2H), 5.37-5.16 (m, 1H), 4.32-4.07 (m, 5H), 4.01-3.90 (m, 2H), 3.33-3.21 (m, 2H), 3.16 (br s, 1H), 3.01-2.85 (m, 3H), 2.52-2.38 (m, 2H), 2.36-2.33 (m, 3H), 2.29 (br d, J=2.8 Hz, 1H), 2.24-2.15 (m, 2H), 2.03 (br d, J=6.0 Hz, 2H), 1.97-1.85 (m, 3H); LCMS (ESI, M+1): m/z=747.2.

### Example 181

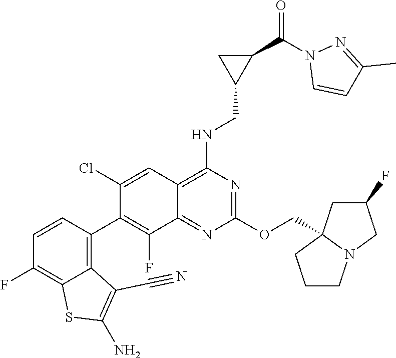 [SMILES: Cc1ccn(C(=O)[C@@H]2C[C@H]2CNc2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-((((trans)-2-(3-methyl-1H-pyrazole-1-carbonyl)cyclopropyl)methyl)amino)quinazolin-7-yl)-7-fluorobenzo[b]thiophene-3-carbonitrile

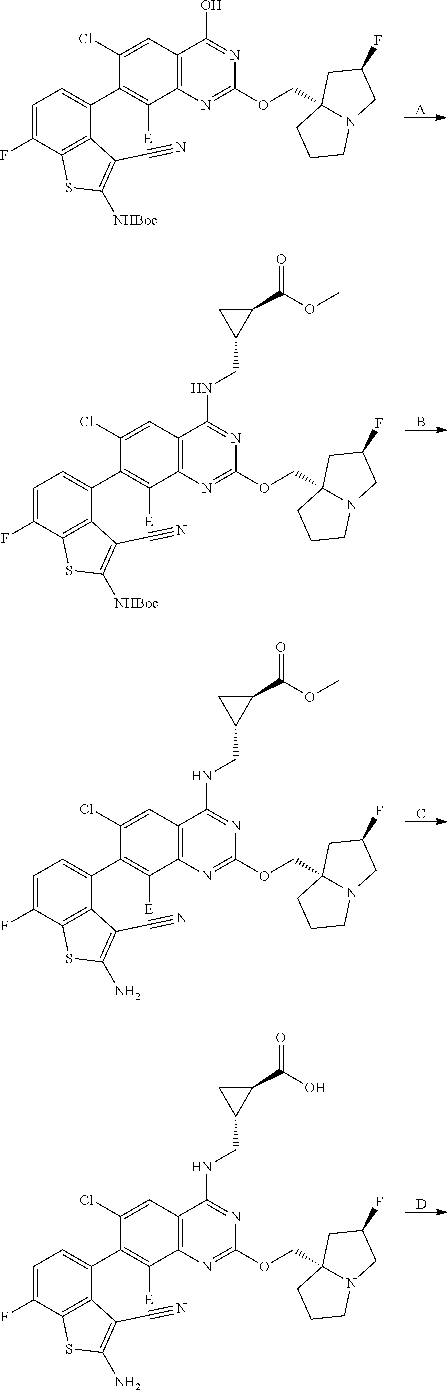

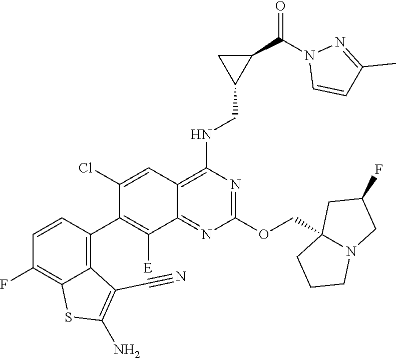

Step A. methyl(1R,2R)-2-(((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylate: To a solution of tert-butyl N-[4-[6-chloro-8-fluoro-2-[[(2R,8S)-2-fluoro-1,2,3,5,6,7-hexahydropyrrolizin-8-yl]methoxy]-4-hydroxy-quinazolin-7-yl]-3-cyano-7-fluoro-benzothiophen-2-yl]carbamate (500 mg, 1.0 equiv) in DMF (10 mL) were added PyBOP (604 mg, 1.5 equiv), methyl(1R,2R)-2-(aminomethyl)cyclopropanecarboxylate (192 mg, 1.5 equiv, HCl) and DIEA (500 mg, 5.0 equiv). The reaction was stirred at 25 °C for 12 hours. The mixture was diluted with water (30 mL) and extracted with EtOAc (20 mL×3). The combined organic layers were washed with brine (30 mL), dried over anhydrous sodium sulfate and concentrated to afford the title compound (600 mg, crude) as yellow solid; LCMS (ESI, M+1): m/z=757.3.

Step B. methyl(1R,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylate: To a solution of methyl(1R,2R)-2-(((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylate (600 mg, 1.0 equiv) in DCM (6 mL) was added TFA (3.07 g, 42 equiv). The reaction was stirred at 25 °C for 0.5 hour. The mixture was concentrated, dissolved in NaHCO₃ (30 mL) and extracted with DCM (20 mL×3). The combined organic layers dried over anhydrous sodium sulfate and concentrated to afford the title compound (400 mg, crude) as yellow solid; LCMS (ESI, M+1): m/z=657.2.

Step C. (1R,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylic acid: To a solution of methyl(1R,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylate (400 mg, 1.0 equiv) in MeOH (5 mL) was added LiOH (1.5 mL, 2 M). The reaction was stirred at 25 °C for 4 hours. The mixture was concentrated and purified with prep-HPLC (column: Waters Xbridge Prep OBD C18 150×40 mm×10 μm; mobile phase: [water (NH₄HCO₃)-ACN]; gradient: 15%-45% B over 20 min) to afford the title compound (200 mg, 50% yield) as off-white solid; LCMS (ESI, M+1): m/z=643.2.

Step D. 2-amino-4-(6-chloro-8-fluoro-2-(((2R,7aS)-2-fluorotetrahydro-1H-pyrrolizin-7a(5H)-yl)methoxy)-4-((((1R,2R)-2-(3-methyl-1H-pyrazole-1-carbonyl)cyclopropyl)methyl)amino)quinazolin-7-yl)-7-fluorobenzo[b]thiophene-3-carbonitrile: To a solution of (1R,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylic acid (50 mg, 1.0 equiv) in DMF (1 mL) were added HATU (44.3 mg, 1.5 equiv), 3-methyl-1H-pyrazole (9.58 mg, 1.5 equiv) and DIEA (30.2 mg, 3.0 equiv). The reaction was stirred at 20 °C for 0.5 hours. The mixture was filtered and purified with prep-HPLC (column: Waters Xbridge 150×25 mm×5 um; mobile phase: [water (NH₄HCO₃)-ACN]; gradient: 48%-78% B over 9 min) to afford the title compound (9.93 mg, 18% yield) as yellow solid; ¹H NMR (400 MHz, CHLOROFORM-d) δ=8.15 (d, J=2.4 Hz, 1H), 7.58 (s, 1H), 7.20-7.14 (m, 1H), 7.06-6.98 (m, 1H), 6.30 (br d, J=2.4 Hz, 2H), 5.63 (br s, 2H), 5.36-5.19 (m, 1H), 4.31-4.23 (m, 1H), 4.21-3.86 (m, 2H), 3.48-3.15 (m, 4H), 3.13-3.05 (m, 1H), 3.02-2.93 (m, 1H), 2.42-2.35 (m, 3H), 2.30-2.24 (m, 1H), 2.23-2.12 (m, 2H), 2.00-1.85 (m, 4H), 1.63 (br dd, J=4.4, 8.4 Hz, 1H), 1.25-1.19 (m, 1H); LCMS (ESI, M+1): m/z=707.3.

### Example 182

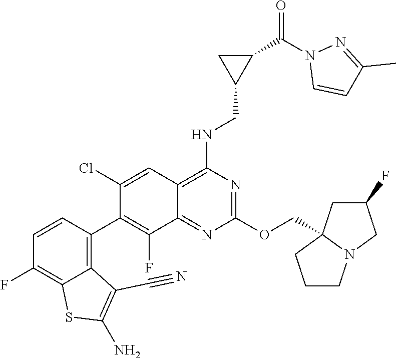 [SMILES: Cc1ccn(C(=O)[C@H]2C[C@H]2CNc2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-((((cis)-2-(3-methyl-1H-pyrazole-1-carbonyl)cyclopropyl)methyl)amino)quinazolin-7-yl)-7-fluorobenzo[b]thiophene-3-carbonitrile

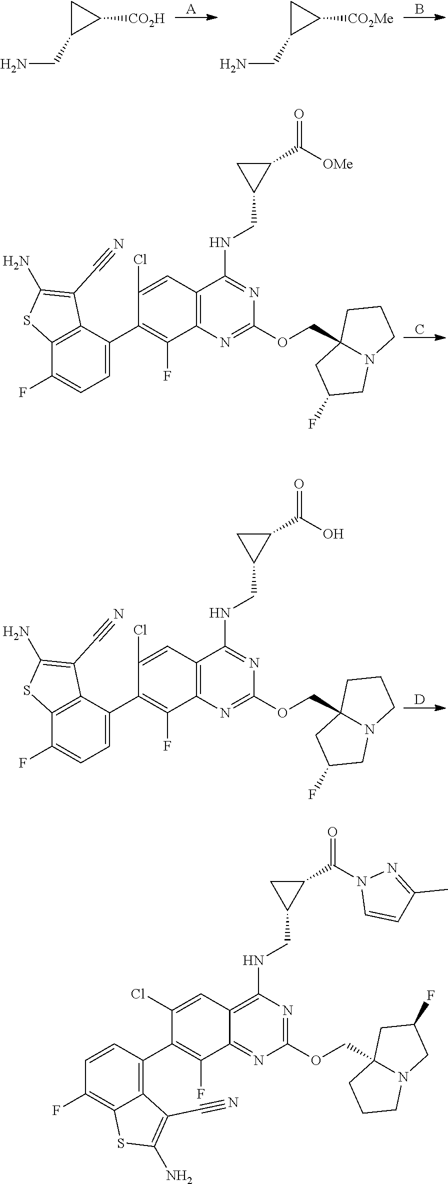

Step A. methyl(1S,2R)-2-(aminomethyl)cyclopropane-1-carboxylate: A solution of (1S,2R)-2-(aminomethyl)cyclopropane-1-carboxylic acid (90.0 mg, 1.0 equiv) in HCl·MeOH (2 mL) were stirred at 25 °C for 10 hours. The mixture was concentrated under reduced pressure to afford the title compound (90.0 mg, crude, HCl) as yellow solid.

Step B. methyl(1S,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (250 mg, 1.0 equiv) in DMSO (3 mL) were added TEA (463 mg, 10 equiv) and PyBOP (357 mg, 1.5 equiv). The reaction was stirred at 25 °C for 0.5 hours. Then methyl(1S,2R)-2-(aminomethyl)cyclopropane-1-carboxylate (88.7 mg, 1.5 equiv, HCl) was added and the reaction was stirred at 25 °C for 1 hour. The mixture was diluted with water (20 mL) and extracted with ethyl acetate (20 mL×2). The combined organic layers were washed with brine (20 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 42%-72% B over 20 min] to afford the title compound (80.0 mg, 26% yield) as yellow solid; LCMS (ESI, M+1): m/z=657.2.

Step C. (1S,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylic acid: To a solution of methyl(1S,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylate (80.0 mg, 1.0 equiv) in MeOH (1 mL) was added LiOH (0.3 mL, 2M). The reaction was stirred at 25 °C for 1 hour. The mixture was filtered and purified with prep-HPLC [column: Waters Xbridge Prep OBD C18 150×40 mm×10 μm; mobile phase: water (NH₄HCO₃)-ACN; gradient: 22%-52% B over 20 min] to afford the title compound (60.0 mg, 76% yield) as yellow solid; LCMS (ESI, M+1): m/z=643.2.

Step D. 2-amino-4-(6-chloro-8-fluoro-2-(((2R,7aS)-2-fluorotetrahydro-1H-pyrrolizin-7a(5H)-yl)methoxy)-4-((((1R,2S)-2-(3-methyl-1H-pyrazole-1-carbonyl)cyclopropyl)methyl)amino)quinazolin-7-yl)-7-fluorobenzo[b]thiophene-3-carbonitrile: To a solution of (1S,2R)-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)cyclopropane-1-carboxylic acid (50.0 mg, 1.0 equiv) in DMF (1 mL) were added 3-methyl-1H-pyrazole (9.58 mg, 1.5 equiv), DIEA (50.2 mg, 5.0 equiv) and HATU (44.3 mg, 1.5 equiv). The reaction was stirred at 25 °C for 0.5 hours. The mixture was filtered and purified with prep-HPLC [column: Waters Xbridge Prep OBD C18 150×40 mm×10 μm; mobile phase: water (NH₄HCO₃)-ACN; gradient: 40%-70% B over 20 min] to afford the title compound (13.1 mg, 23% yield) as pink solid; ¹H NMR (400 MHz, DMSO-d₆) δ=8.72-8.61 (m, 1H), 8.30-8.24 (m, 1H), 8.21 (d, J=6.4 Hz, 1H), 8.15-8.05 (m, 2H), 7.23-7.18 (m, 1H), 7.17-7.10 (m, 1H), 6.42 (dd, J=2.8, 14.4 Hz, 1H), 5.39-5.16 (m, 1H), 4.11-4.04 (m, 1H), 4.02-3.95 (m, 1H), 3.70 (br t, J=6.0 Hz, 2H), 3.25-3.17 (m, 1H), 3.08 (br d, J=12.8 Hz, 2H), 3.05-2.99 (m, 1H), 2.86-2.78 (m, 1H), 2.26-2.22 (m, 3H), 2.18-2.11 (m, 2H), 2.07-2.00 (m, 2H), 1.86-1.75 (m, 3H), 1.44-1.38 (m, 1H), 1.36-1.31 (m, 1H); LCMS (ESI, M+1): m/z=707.2.

### Example 183

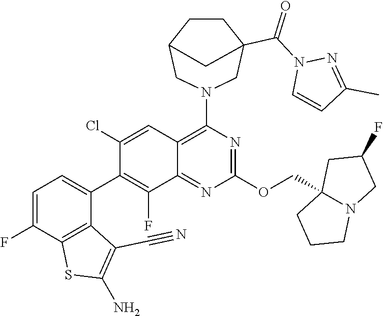 [SMILES: Cc1ccn(C(=O)C23CCC(CN(c4nc(OC[C@@]56CCCN5C[C@H](F)C6)nc5c(F)c(-c6ccc(F)c7sc(N)c(C#N)c67)c(Cl)cc45)C2)C3)n1]

### 2-amino-4-(6-chloro-8-fluoro-2-(((2R,7aS)-2-fluorotetrahydro-1H-pyrrolizin-7a(5H)-yl)methoxy)-4-(1-(3-methyl-1H-pyrazole-1-carbonyl)-3-azabicyclo[3.2.1]octan-3-yl)quinazolin-7-yl)-7-fluorobenzo[b]thiophene-3-carbonitrile

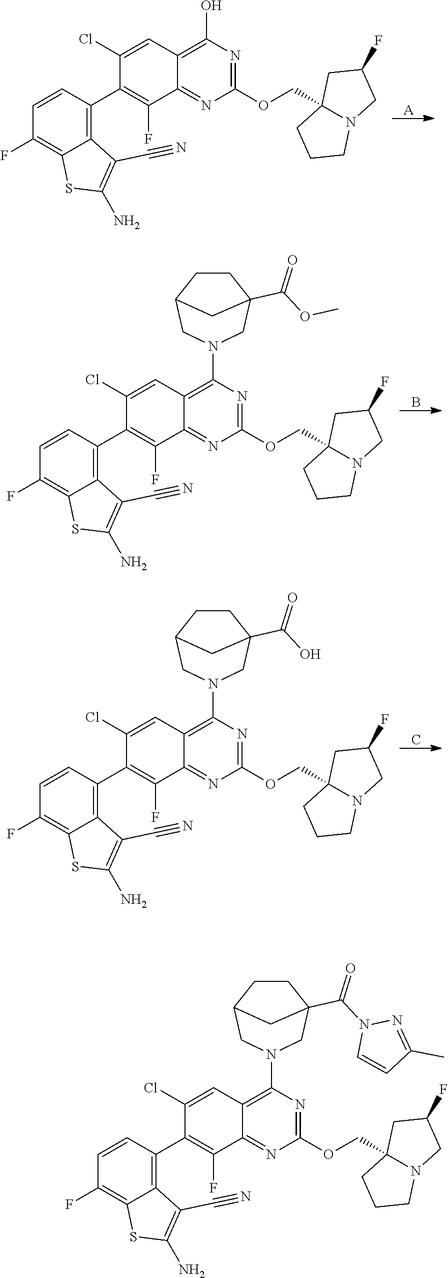

Step A. methyl 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.1]octane-1-carboxylate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (150 mg, 1.0 equiv) and DIEA (213 mg, 6.0 equiv) in DMF (1.5 mL) were added PyBOP (214 mg, 1.5 equiv) and methyl 3-azabicyclo[3.2.1]octane-1-carboxylate (67.8 mg, 1.2 equiv, HCl). The reaction was stirred at 25 °C for 0.5 hours. The mixture was filtered and purified by prep-HPLC [Waters Xbridge Prep OBD C18 150×40 mm×10 μm; A: water (NH₄HCO₃); B: ACN, B %: 50%-80% over 20 min] to afford the title compound (100 mg, 52% yield) as a yellow solid; LCMS (ESI, M+1): m/z=697.3.

Step B. 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.1]octane-1-carboxylic acid: To a solution of methyl 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.1]octane-1-carboxylate (70.0 mg, 1.0 equiv) in MeOH (2 mL) was added LiOH·H₂O (2 M, 2 mL, 40 equiv). The reaction was stirred at 40 °C for 1 hour. The mixture was concentrated, adjusted to pH=7 using FA and purified by reversed-phase flash (0.1% FA condition) to afford the title compound (65.0 mg, 86% yield, HCOOH) as a yellow solid; LCMS (ESI, M+1): m/z=683.2.

Step C. 2-amino-4-(6-chloro-8-fluoro-2-(((2R,7aS)-2-fluorotetrahydro-1H-pyrrolizin-7a(5H)-yl)methoxy)-4-(1-(3-methyl-1H-pyrazole-1-carbonyl)-3-azabicyclo[3.2.1]octan-3-yl)quinazolin-7-yl)-7-fluorobenzo[b]thiophene-3-carbonitrile: To a solution of 3-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-azabicyclo[3.2.1]octane-1-carboxylic acid (60.0 mg, 1.0 equiv, HCOOH) and 3-methyl-1H-pyrazole (20.3 mg, 3.0 equiv) in DMF (1 mL) were added DIEA (63.8 mg, 6.0 equiv) and HATU (62.6 mg, 2.0 equiv). The reaction was stirred at 25 °C for 0.5 hours. The mixture was filtered. The crude product was purified by reversed-phase flash (0.1% FA condition) to afford the title compound (5.84 mg, 8.6% yield) was obtained as a yellow solid; ¹H NMR (400 MHz, CHLOROFORM-d) δ=8.19 (br s, 1H), 8.05-7.79 (m, 1H), 7.23-7.14 (m, 1H), 7.08-6.94 (m, 1H), 6.22 (d, J=2.8 Hz, 1H), 5.64-5.30 (m, 2H), 5.25-5.02 (m, 1H), 4.32-4.16 (m, 2H), 3.60-3.39 (m, 1H), 3.34-3.12 (m, 3H), 3.06-2.84 (m, 1H), 2.57-2.50 (m, 1H), 2.47-2.39 (m, 1H), 2.36-2.31 (m, 2H), 2.30-2.24 (m, 2H), 2.24-2.13 (m, 3H), 2.05-2.00 (m, 1H), 2.00-1.72 (m, 9H); LCMS (ESI, M+1): m/z=747.4.

### Example 184

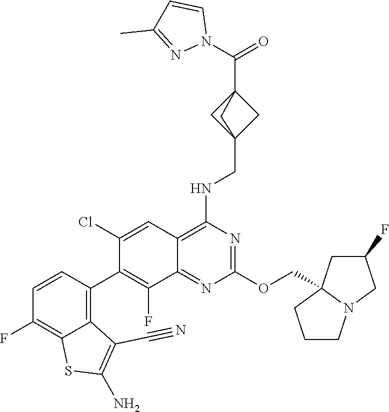 [SMILES: Cc1ccn(C(=O)C23CC(CNc4nc(OC[C@@]56CCCN5C[C@H](F)C6)nc5c(F)c(-c6ccc(F)c7sc(N)c(C#N)c67)c(Cl)cc45)(C2)C3)n1]

### 2-amino-4-(6-chloro-8-fluoro-2-(((2R,7aS)-2-fluorotetrahydro-1H-pyrrolizin-7a(5H)-yl)methoxy)-4-(((3-(3-methyl-1H-pyrazole-1-carbonyl)bicyclo[1.1.1]pentan-1-yl)methyl)amino)quinazolin-7-yl)-7-fluorobenzo[b]thiophene-3-carbonitrile

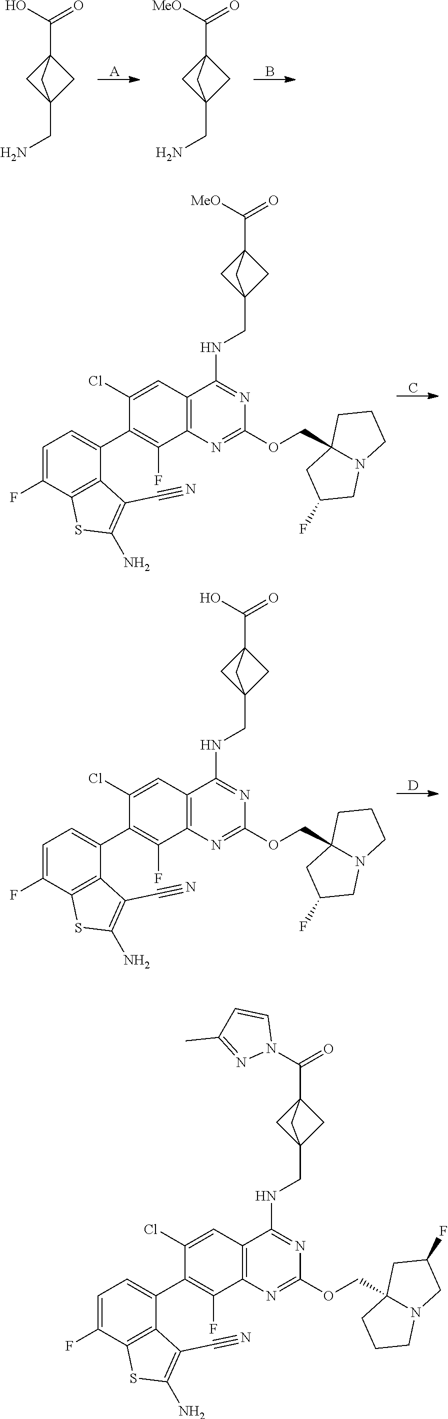

Step A. methyl 3-(aminomethyl)bicyclo[1.1.1]pentane-1-carboxylate: A solution of 3-(aminomethyl)bicyclo[1.1.1]pentane-1-carboxylic acid (100 mg, 1.0 equiv) in HCl·MeOH (2 mL) was stirred at 25 °C for 12 hours. The mixture was concentrated under reduced pressure to afford the title compound (100 mg, crude, HCl) as yellow oil; ¹H NMR (400 MHz, METHANOL-d₄) δ=3.68 (s, 2H), 3.07 (s, 3H), 2.08 (s, 6H).

Step B. methyl 3-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)bicyclo[1.1.1]pentane-1-carboxylate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (170 mg, 1.0 equiv) in DMSO (4 mL) were added PyBOP (243 mg, 1.5 equiv) and TEA (157 mg, 5.0 equiv). The reaction was stirred at 25 °C for 0.5 hours. Then methyl 3-(aminomethyl)bicyclo[1.1.1]pentane-1-carboxylate (96.6 mg, 2.0 equiv, HCl) was added and the reaction was stirred at 25 °C for 0.5 hours. The mixture was diluted with water (10 mL) and extracted with ethyl acetate (10 mL×2). The combined organic layers were washed with brine (20 mL), dried over anhydrous sodium sulfate, concentrated and purified with prep-HPLC [neutral condition; column: Waters Xbridge Prep OBD C18 150×40 mm×10 μm; mobile phase: water (NH₄HCO₃)-ACN; gradient: 43%-73% B over 20 min] to afford the title compound (50.0 mg, 23% yield) as yellow solid; LCMS (ESI, M+1): m/z=683.2.

Step C. 3-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)bicyclo[1.1.1]pentane-1-carboxylic acid: To a solution of methyl 3-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)bicyclo[1.1.1]pentane-1-carboxylate (50.0 mg, 1.0 equiv) in MeOH (1 mL) was added LiOH (0.18 mL, 2M). The reaction was stirred at 25 °C for 1 hour. The mixture was diluted with water (10 mL) and extracted with ethyl acetate (10 mL×2). The combined organic layers were washed with brine (10 mL), dried over anhydrous sodium sulfate, concentrated and purified with prep-HPLC [FA condition; column: YMC-Actus Triart C18 150×30 mm×7 μm; mobile phase: water (FA)-ACN; gradient: 23%-53% B over 10 min] to afford the title compound (30.0 mg, 61% yield) as white solid; LCMS (ESI, M+1): m/z=669.4.

Step D. 2-amino-4-(6-chloro-8-fluoro-2-(((2R,7aS)-2-fluorotetrahydro-1H-pyrrolizin-7a(5H)-yl)methoxy)-4-(((3-(3-methyl-1H-pyrazole-1-carbonyl)bicyclo[1.1.1]pentan-1-yl)methyl)amino)quinazolin-7-yl)-7-fluorobenzo[b]thiophene-3-carbonitrile: To a solution of 3-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)bicyclo[1.1.1]pentane-1-carboxylic acid (30.0 mg, 1.0 equiv) in DMF (1 mL) were added 3-methyl-1H-pyrazole (5.52 mg, 1.5 equiv), DIEA (29.0 mg, 5.0 equiv) and HATU (25.6 mg, 1.5 equiv). The reaction was stirred at 25 °C for 1 hour. The mixture was diluted with water (10 mL) and extracted with ethyl acetate (10 mL×2). The combined organic layers were washed with brine (10 mL), dried over anhydrous sodium sulfate, concentrated and purified with prep-HPLC [neutral condition; column: Waters Xbridge Prep OBD C18 150×40 mm×10 μm; mobile phase: water (NH₄HCO₃)-ACN; gradient: 48%-78% B over 20 min]. to afford the title compound (6.49 mg, 18% yield) as off-white solid; ¹H NMR (400 MHz, CHLOROFORM-d) δ=8.12 (d, J=2.8 Hz, 1H), 7.59 (s, 1H), 7.19 (dd, J=5.2, 8.4 Hz, 1H), 7.03 (t, J=8.8 Hz, 1H), 6.22 (d, J=2.8 Hz, 1H), 5.78 (br d, J=3.2 Hz, 1H), 5.48 (br s, 2H), 5.37-5.19 (m, 1H), 4.31-4.24 (m, 1H), 4.17 (dd, J=4.0, 10.2 Hz, 1H), 3.96 (td, J=5.6, 14.0 Hz, 1H), 3.66 (td, J=4.8, 14.4 Hz, 1H), 3.32-3.23 (m, 2H), 3.17 (s, 1H), 3.03-2.94 (m, 1H), 2.40-2.34 (m, 6H), 2.33 (s, 3H), 2.29 (br d, J=3.2 Hz, 1H), 2.23-2.16 (m, 2H), 1.98-1.88 (m, 3H); LCMS (ESI, M+1): m/z=733.4.

### Example 185

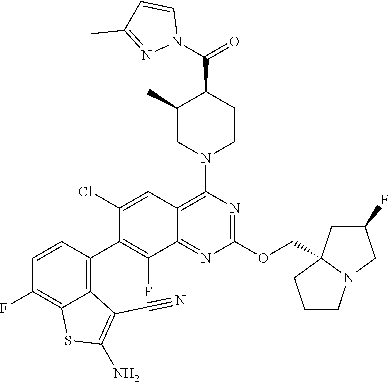 [SMILES: Cc1ccn(C(=O)[C@H]2CCN(c3nc(OC[C@@]45CCCN4C[C@H](F)C5)nc4c(F)c(-c5ccc(F)c6sc(N)c(C#N)c56)c(Cl)cc34)C[C@H]2C)n1]

### 2-amino-4-(6-chloro-8-fluoro-2-(((2R,7aS)-2-fluorotetrahydro-1H-pyrrolizin-7a(5H)-yl)methoxy)-4-((cis)-3-methyl-4-(3-methyl-1H-pyrazole-1-carbonyl)piperidin-1-yl)quinazolin-7-yl)-7-fluorobenzo[b]thiophene-3-carbonitrile

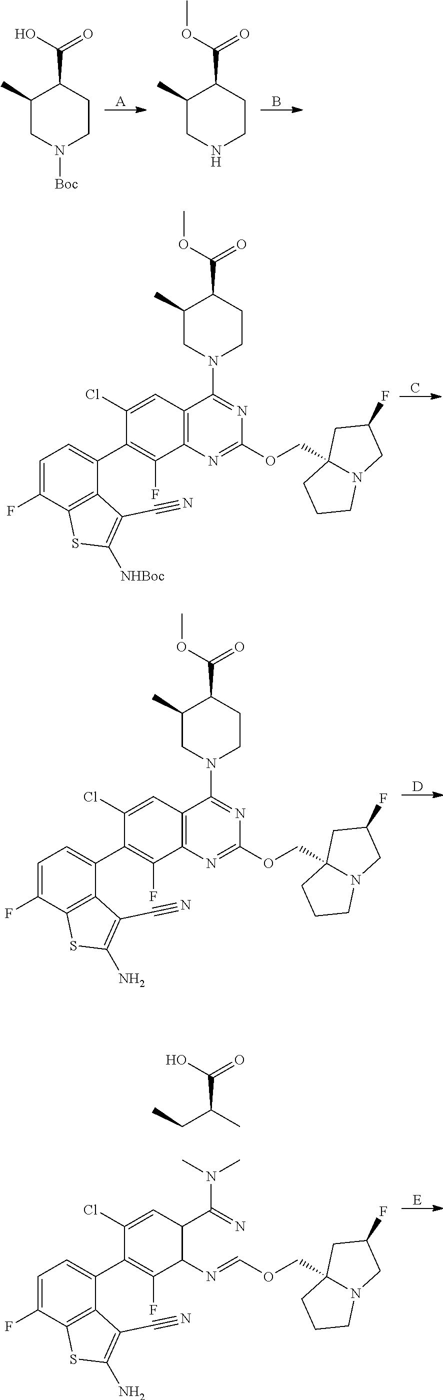

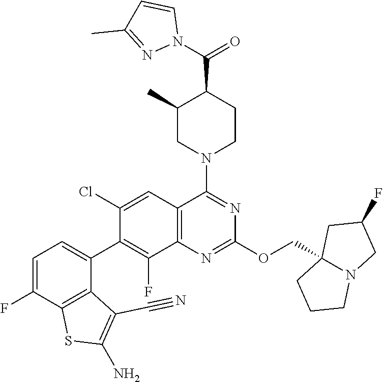 [SMILES: Cc1ccn(C(=O)[C@H]2CCN(c3nc(OC[C@@]45CCCN4C[C@H](F)C5)nc4c(F)c(-c5ccc(F)c6sc(N)c(C#N)c56)c(Cl)cc34)C[C@H]2C)n1]

Step A. methyl(3S,4S)-3-methylpiperidine-4-carboxylate: A mixture of (3S,4S)-1-(tert-butoxycarbonyl)-3-methylpiperidine-4-carboxylic acid (350 mg, 1.0 equiv) and HCl·MeOH (2 M, 3.50 mL) was stirred at 25 °C for 1 hour. The mixture was concentrated to afford the title compound (250 mg, crude, HCl salt) as yellow solid.

Step B. methyl(3S,4S)-1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-methylpiperidine-4-carboxylate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv) and methyl(3S,4S)-3-methylpiperidine-4-carboxylate (250 mg, 1.67 equiv, HCl salt) in DMF (5 mL) were added PyBOP (604 mg, 1.5 equiv) and DIEA (1.00 g, 10 equiv). The reaction was stirred at 25 °C for 1 hour. The mixture was filtered and the filtrate was purified by reversed phase flash chromatography [C18, 0.1% formic acid condition] to afford the title compound (170 mg, 20% yield) as yellow solid; LCMS (ESI, M+1): m/z=785.2.

Step C. methyl(3S,4S)-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-methylpiperidine-4-carboxylate: To a solution of methyl (3S,4S)-1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-methylpiperidine-4-carboxylate (170 mg, 1.0 equiv) in DCM (1.7 mL) was added TFA (1.7 mL, 106 equiv). The reaction was stirred at 25 °C for 0.5 hours. The mixture was concentrated to afford the title compound (148 mg, crude, CF₃COOH salt) as yellow oil.

Step D. (3S,4S)-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-methylpiperidine-4-carboxylic acid: To a solution of methyl(3S,4S)-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-methylpiperidine-4-carboxylate (148 mg, 1.0 equiv, CF₃COOH salt) in MeOH (1.48 mL) was added LiOH·H₂O (2 M in H₂O, 1.48 mL, 13.7 equiv). The reaction was stirred at 25 °C for 8 hours. The mixture was filtered and the filtrate was purified by reversed phase flash chromatography [C18, 0.1% formic acid condition] to afford the title compound (90 mg, 58% yield) as white solid; LCMS (ESI, M+1): m/z=671.1.

Step E. 2-amino-4-(6-chloro-8-fluoro-2-(((2R,7aS)-2-fluorotetrahydro-1H-pyrrolizin-7a(5H)-yl)methoxy)-4-((3S,4S)-3-methyl-4-(3-methyl-1H-pyrazole-1-carbonyl)piperidin-1-yl)quinazolin-7-yl)-7-fluorobenzo[b]thiophene-3-carbonitrile: To a solution of (3S,4S)-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-methylpiperidine-4-carboxylic acid (80 mg, 1.0 equiv) and 3-methyl-1H-pyrazole (48.9 mg, 5 equiv) in DMF (0.8 mL) were added HATU (90.6 mg, 2.0 equiv) and DIEA (77.0 mg, 5.0 equiv). The reaction was stirred at 25 °C for 0.5 hour. The mixture was filtered and purified by reversed phase flash chromatography [C18, H₂O condition]. The desired fractions were diluted with water (50 mL) and extracted with dichloromethane (2×30 mL). The organic layers were dried over anhydrous sodium sulfate and concentrated to afford the title compound (17.8 mg, 20% yield) as yellow solid; ¹H NMR (400 MHz, CHLOROFORM-d) δ=8.18 (br s, 1H), 7.78 (br s, 1H), 7.23-7.18 (m, 1H), 7.03 (br s, 1H), 6.28 (br s, 1H), 5.53-5.36 (m, 2H), 5.36-5.16 (m, 1H), 4.41-4.10 (m, 5H), 3.78-3.63 (m, 1H), 3.58-3.40 (m, 1H), 3.33-3.13 (m, 3H), 3.03-2.88 (m, 1H), 2.74-2.60 (m, 1H), 2.48-2.30 (m, 4H), 2.29-2.10 (m, 3H), 2.01-1.85 (m, 4H), 1.08-0.94 (m, 3H); LCMS (ESI, M+1): m/z=735.2.

### Example 186

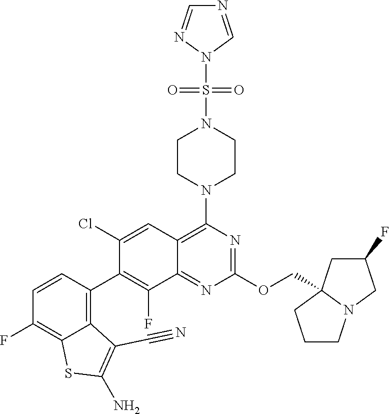 [SMILES: N#Cc1c(N)sc2c(F)ccc(-c3c(Cl)cc4c(N5CCN(S(=O)(=O)n6cncn6)CC5)nc(OC[C@@]56CCCN5C[C@H](F)C6)nc4c3F)c12]

### 4-(4-(4-((1H-1,2,4-triazol-1-yl)sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

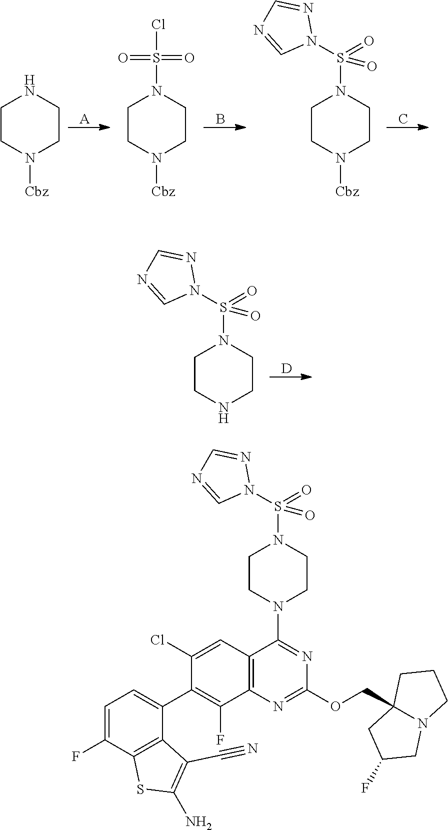

Step A. benzyl 4-(chlorosulfonyl)piperazine-1-carboxylate; To a solution of sulfuryl dichloride (1.46 g, 2.5 equiv) in acetonitrile (20.0 mL) was added benzyl piperazine-1-carboxylate (950 mg, 1.0 equiv) at 0 °C, then triethylamine (1.16 g, 2.7 equiv) was added dropwise at 0 °C and stirred for 0.5 hour. The mixture was heated to 50 °C for 2 hours. The mixture was quenched with saturated bicarbonate aqueous solution (50.0 mL) and extracted with ethyl acetate (2×30 mL). The combined organic layers were washed with brine (2×20 mL), dried over anhydrous sodium sulfate, concentrated to afford the title compound (1.35 g, 98% yield) as a yellow oil; ¹H NMR (400 MHz, chloroform-d) δ=7.45-7.32 (m, 5H), 5.17 (s, 2H), 3.73 (br s, 4H), 3.47-3.01 (m, 4H).

Step B. benzyl 4-((1H-1,2,4-triazol-1-yl)sulfonyl)piperazine-1-carboxylate: A solution of 1H-1,2,4-triazole (450 mg, 1.0 equiv), benzyl 4-chlorosulfonylpiperazine-1-carboxylate (2.08 g, 1.00 equiv) in toluene (16.0 mL) was added triethylamine (660 mg, 1.00 equiv), and the mixture was stirred at 50 °C for 16 hours. The mixture was directly concentrated to give a residue and then purified by column chromatography (SiO₂, petroleum ether/ethyl acetate=5/1 to 1/1) to afford the title compound (1.19 g, 52% yield) as an colorless oil; ¹H NMR (400 MHz, chloroform-d) δ=8.59 (s, 1H), 8.08 (s, 1H), 7.40-7.33 (m, 5H), 5.13 (s, 2H), 3.78-3.59 (m, 4H), 3.47-3.32 (m, 4H).

Step C. 1-((1H-1,2,4-triazol-1-yl)sulfonyl)piperazine: To a solution of benzyl 4-(1,2,4-triazol-1-ylsulfonyl)piperazine-1-carboxylate (1.19 g, 1.0 equiv) in ethyl alcohol (10.0 mL) was added Pd(OH)₂ (1.00 g, 20% purity, 0.42 equiv) under a nitrogen atmosphere. The suspension was degassed under vacuum and purged with hydrogen several times, then mixture was stirred under hydrogen (15 psi) at 25 °C for 1.5 hours. The reaction mixture was filtered and the filter was concentrated to afford the title compound (700 mg, 95% yield) as a colorless oil which was used directly; ¹H NMR (400 MHz, chloroform-d) δ=8.71-8.52 (m, 1H), 8.17-7.95 (m, 1H), 3.45-3.32 (m, 4H), 3.06-2.89 (m, 4H)

Step D. 4-(4-(4-((1H-1,2,4-triazol-1-yl)sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (40.0 mg, 1.00 equiv) and 1-(1,2,4-triazol-1-ylsulfonyl)piperazine (55.0 mg, 3.5 equiv) in dimethylformamide (0.50 mL) was added diisopropylethylamine (28.0 mg, 3.0 equiv) and benzotriazol-1-yloxy(tripyrrolidin-1-yl)phosphanium;hexafluorophosphate (50.0 mg, 1.3 equiv). The mixture was stirred at 25 °C for 12 hours. The reaction mixture was purified by prep-HPLC (FA condition; column: Waters Xbridge 150×25 mm×5 μm; mobile phase: [water (NH₄HCO₃)-ACN]; gradient: 45%-75% B over 15 min) and lyophilized to afford the title compound (12.1 mg, 22% yield) as a white solid; ¹H NMR (400 MHz, methanol-d₄) δ=9.00 (s, 1H), 8.21 (s, 1H), 7.85 (d, J=1.2 Hz, 1H), 7.19 (dd, J=4.8, 8.4 Hz, 1H), 7.03 (dd, J=8.4, 9.2 Hz, 1H), 5.42-5.18 (m, 1H), 4.32-4.17 (m, 2H), 4.08-3.90 (m, 4H), 3.66 (br t, J=4.8 Hz, 4H), 3.29-3.15 (m, 3H), 3.06-2.97 (m, 1H), 2.42-2.18 (m, 2H), 2.18-2.08 (m, 1H), 2.06-1.94 (m, 2H), 1.94-1.81 (m, 1H); LCMS (ESI, M+1): m/z=745.3

### Example 187

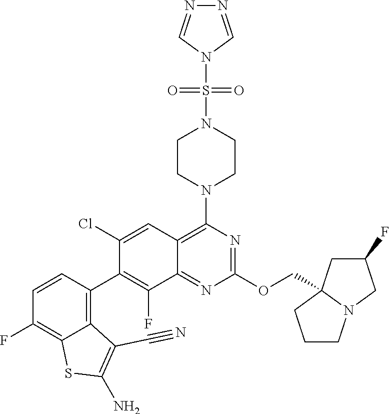 [SMILES: N#Cc1c(N)sc2c(F)ccc(-c3c(Cl)cc4c(N5CCN(S(=O)(=O)n6cnnc6)CC5)nc(OC[C@@]56CCCN5C[C@H](F)C6)nc4c3F)c12]

### 4-(4-(4-((4H-1,2,4-triazol-4-yl)sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

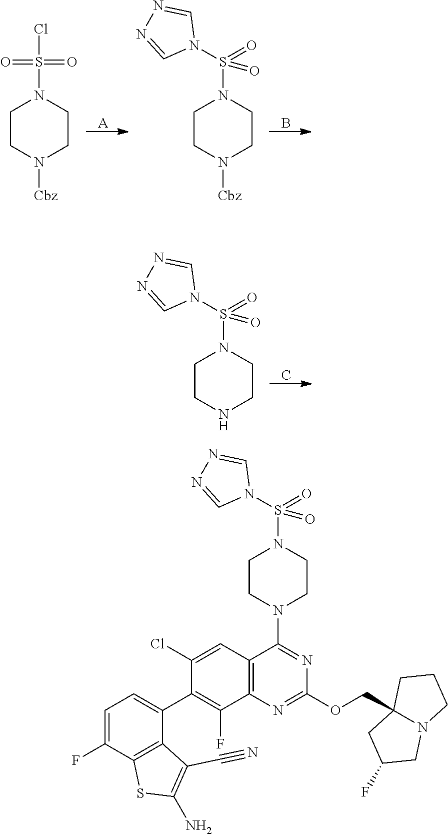

Step A. benzyl 4-((4H-1,2,4-triazol-4-yl)sulfonyl)piperazine-1-carboxylate: To a solution of benzyl 4-chlorosulfonylpiperazine-1-carboxylate (3.00 g, 1.0 equiv) 4H-1,2,4-triazole (650 mg, 1.0 equiv) in toluene (30 mL) was added triethylamine (952 mg, 1.31 mL, 1.0 equiv). The mixture was stirred at 50 °C for 12 hours. The reaction mixture was directly concentrated and purified by column chromatography (SiO₂, petroleum ether/ethyl acetate=1/1 to 2/1) to afford the title compound (400 mg, 12% yield) as a white solid; ¹H NMR (400 MHz, chloroform-d) δ=8.43 (s, 2H), 7.39-7.30 (m, 5H), 5.12 (s, 2H), 3.75-3.62 (m, 4H), 3.23 (br d, J=0.8 Hz, 4H).

Step B. 1-((4H-1,2,4-triazol-4-yl)sulfonyl)piperazine: To a solution of benzyl 4-((4H-1,2,4-triazol-4-yl)sulfonyl)piperazine-1-carboxylate (430 mg, 1.0 equiv) in ethyl alcohol (5.00 mL) was added Pd(OH)₂ (430 mg, 20% purity, 0.5 equiv) under a nitrogen atmosphere. The suspension was degassed under vacuum and purged with hydrogen several times, then mixture was stirred under hydrogen (15 psi) at 25 °C for 1.5 hours. The reaction mixture was filtered and the filtrate was concentrated to give a title compound (200 mg, 75% yield) as a yellow solid; ¹H NMR (400 MHz, chloroform-d) δ=8.45 (s, 2H), 3.24-3.20 (m, 4H), 3.01-2.96 (m, 4H).

Step C. 4-(4-(4-((4H-1,2,4-triazol-4-yl)sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (80 mg, 1.0 equiv), 1-((4H-1,2,4-triazol-4-yl)sulfonyl)piperazine (96 mg, 3.0 equiv) in dimethylformamide (1.00 mL) was added benzotriazol-1-yloxy(tripyrrolidin-1-yl)phosphanium;hexafluorophosphate (114 mg, 1.5 equiv) and diisopropylethylamine (76 mg, 4.0 equiv). The mixture was stirred at 25 °C for 12 hours. The reaction mixture was concentrated and then collected the cake by filtration. The residue was purified by prep-HPLC (neutral condition; column: Waters Xbridge 150×25 mm×5 μm; mobile phase: [water (NH₄HCO₃)-ACN]; gradient: 35%-65% B over 15 min) to afford the title compound (5.8 mg, 5.3% yield); ¹H NMR (400 MHz, methanol-d₄) δ=9.02 (s, 2H), 7.87 (s, 1H), 7.19 (dd, J=5.2, 8.4 Hz, 1H), 7.03 (t, J=8.8 Hz, 1H), 5.40-5.19 (m, 1H), 4.30-4.24 (m, 1H), 4.23-4.16 (m, 1H), 4.07-3.93 (m, 4H), 3.66-3.54 (m, 4H), 3.28-3.16 (m, 3H), 3.05-2.95 (m, 1H), 2.34-2.09 (m, 3H), 2.03-1.85 (m, 3H); LCMS (ESI, M+1): m/z=745.3.

### Example 188

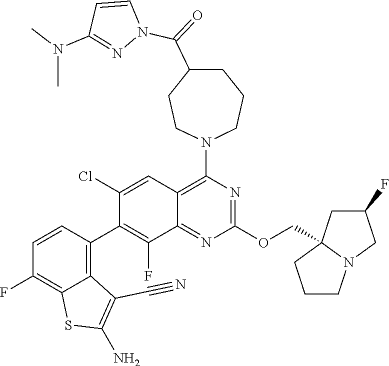 [SMILES: CN(C)c1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-4-(4-(3-(dimethylamino)-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

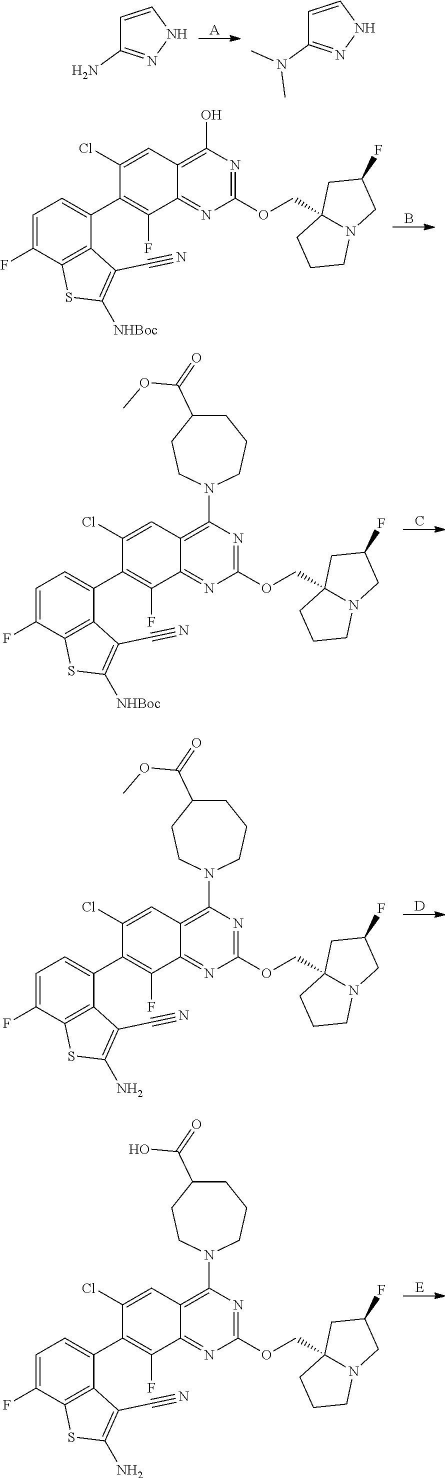

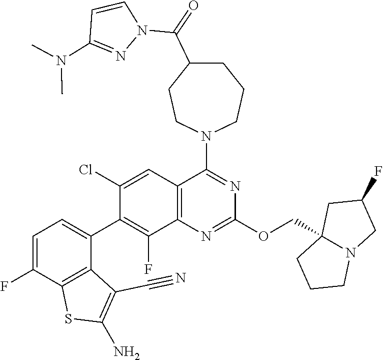 [SMILES: CN(C)c1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

Step A. N,N-dimethyl-1H-pyrazol-3-amine: To a solution of 1H-pyrazol-3-amine (1.00 g, 1.0 equiv) in MeOH (30 mL) was added formaldehyde (1.50 g, 4.1 equiv) at 0 °C. The reaction was stirred at 25 °C for 1 hour. NaBH₃CN (1.89 g, 2.5 equiv) was added into above mixture at 0 °C. The reaction was stirred at 25 °C for 14 hours. The mixture was diluted with water (30 mL) and extracted with EtOAc (4×20 mL). The combined organic layers were washed with brine (40 mL), dried over anhydrous sodium sulfate, concentrated and purified with reversed phase flash [water (0.1% formic acid)/acetonitrile=19/1] to afford the title compound (560 mg, 41% yield) as light yellow oil; ¹H NMR (400 MHz, DMSO-d₆) δ=7.40 (d, J=1.6 Hz, 1H), 5.59 (d, J=1.6 Hz, 1H), 2.72 (s, 6H).

Step B. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (480 mg, 1.0 equiv) and methyl azepane-4-carboxylate (415 mg, 3.0 equiv) in DMSO (2 mL) were added (Benzotriazol-1-yloxy)tripyrrolidinophosphonium hexafluorophosphate (853 mg, 2.0 equiv) and TEA (277 mg, 3.0 equiv). The reaction was stirred at 25 °C for 14 hours. The mixture was purified with reversed phase flash chromatography [water (0.1% NH₃·H₂O)/acetonitrile=1/4] to afford the title compound (320 mg, 54% yield) as yellow solid; LCMS (ESI, M+1): m/z=785.2.

Step C. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (310 mg, 1.0 equiv) in DCM (3 mL) was added TFA (4.5 mL) dropwise at 0 °C The reaction was stirred at 20 °C for 1 hour. The mixture was concentrated. The residue was dissolved in EtOAc (15 mL) and saturated NaHCO₃ aqueous (15 mL) at 0 °C. The mixture was extracted with EtOAc (4×10 mL). The combined organic layers were washed brine (20 mL), dried over anhydrous sodium sulfate and concentrated to afford the title compound (320 mg, crude) as yellow solid; LCMS (ESI, M+1): m/z=685.3

Step D. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (320 mg, 1.0 equiv) in MeOH (3.5 mL) was added LiOH·H₂O (1.5 M in H₂O, 934 μL, 3.0 equiv). The reaction was stirred at 20 °C for 2 hours. The pH of the mixture was adjusted to 8 with 1N HCl at 0 °C. The mixture was purified with reversed phase flash chromatography [water (0.1% formic acid)/acetonitrile=1/1] to afford the title compound (170 mg, 62% yield over two steps) as yellow solid; LCMS (ESI, M+1): m/z=671.2.

Step E. 2-amino-4-(6-chloro-4-(4-(3-(dimethylamino)-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of 1-(7-fluorotetrahydro-1H-pyrrolizin-7a(5H)-yl)methoxy)quinazolin-4-yl)azepane-4-carboxylic acid (100 mg, 1.0 equiv) and N,N-dimethyl-1H-pyrazol-3-amine (41.4 mg, 2.5 equiv) in DMAc (1 mL) were added HATU (85.0 mg, 1.5 equiv) and DIEA (57.8 mg, 3.0 equiv). The reaction was stirred at 25 °C for 14 hours. The mixture was purified with prep-HPLC [column: Waters Xbridge 150×25 mm×10 μm; mobile phase: water (NH₄HCO₃)-acetonitrile; gradient: 70%-90% B over 8 minutes] to afford the title compound (55.8 mg, 48% yield) as white solid; ¹H NMR (400 MHz, DMSO-d₆) δ=8.18-8.06 (m, 3H), 8.01 (s, 1H), 7.29-7.19 (m, 1H), 7.18-7.12 (m, 1H), 6.28 (dd, J=3.2, 4.8 Hz, 1H), 5.37-5.14 (m, 1H), 4.28-4.06 (m, 3H), 4.06-3.94 (m, 2H), 3.88-3.71 (m, 1H), 3.68-3.48 (m, 1H), 3.13-2.96 (m, 3H), 2.84 (s, 3H), 2.81 (s, 4H), 2.35-2.22 (m, 1H), 2.19-1.93 (m, 7H), 1.88-1.60 (m, 4H); LCMS (ESI, M+1): m/z=764.4.

### Example 189

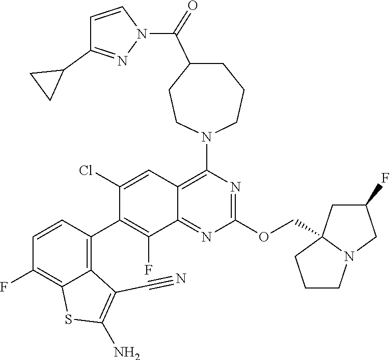 [SMILES: N#Cc1c(N)sc2c(F)ccc(-c3c(Cl)cc4c(N5CCCC(C(=O)n6ccc(C7CC7)n6)CC5)nc(OC[C@@]56CCCN5C[C@H](F)C6)nc4c3F)c12]

### 2-amino-4-(6-chloro-4-(4-(3-cyclopropyl-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

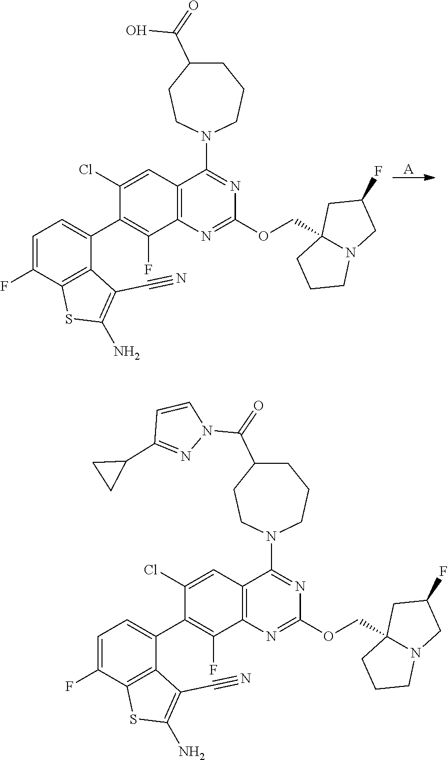

Step A. 2-amino-4-(6-chloro-4-(4-(3-cyclopropyl-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (100 mg, 1.0 equiv) and 3-cyclopropyl-1H-pyrazole (40.3 mg, 2.5 equiv) in DMAc (1 mL) were added TEA (67.9 mg, 4.5 equiv), EDCI (51.4 mg, 1.8 equiv) and HOBt (38.3 mg, 1.9 equiv). The reaction was stirred at 25 °C for 14 hours. The mixture was purified with prep-HPLC [column: Waters Xbridge 150×25 mm×10 μm; mobile phase: water (NH₄HCO₃)-acetonitrile; gradient: 75%-95% B over 8 minutes] to afford the title compound (27.9 mg, 24% yield) as white solid; ¹H NMR (400 MHz, DMSO-d₆) δ=8.23 (t, J=2.4 Hz, 1H), 8.10 (s, 2H), 8.00 (s, 1H), 7.24 (ddd, J=5.2, 8.4, 14.4 Hz, 1H), 7.19-7.11 (m, 1H), 6.34 (dd, J=2.8, 4.4 Hz, 1H), 5.33-5.16 (m, 1H), 4.29-3.65 (m, 7H), 3.15-2.94 (m, 3H), 2.88-2.75 (m, 1H), 2.36-2.23 (m, 1H), 2.19-1.92 (m, 8H), 1.88-1.58 (m, 4H), 1.02-0.86 (m, 2H), 0.80-0.59 (m, 2H); LCMS (ESI, M+1): m/z=761.2.

### Example 190

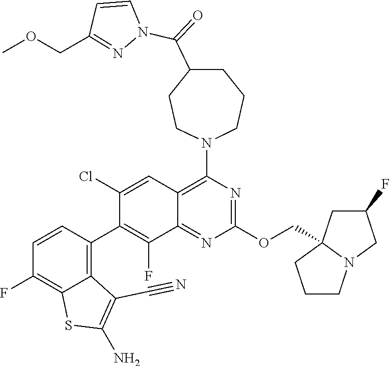 [SMILES: COCc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-8-fluoro-2-(((2R,7aS)-2-fluorotetrahydro-1H-pyrrolizin-7a(5H)-yl)methoxy)-4-(4-(3-(methoxymethyl)-1H-pyrazole-1-carbonyl) azepan-1-yl)quinazolin-7-yl)-7-fluorobenzo[b]thiophene-3-carbonitrile

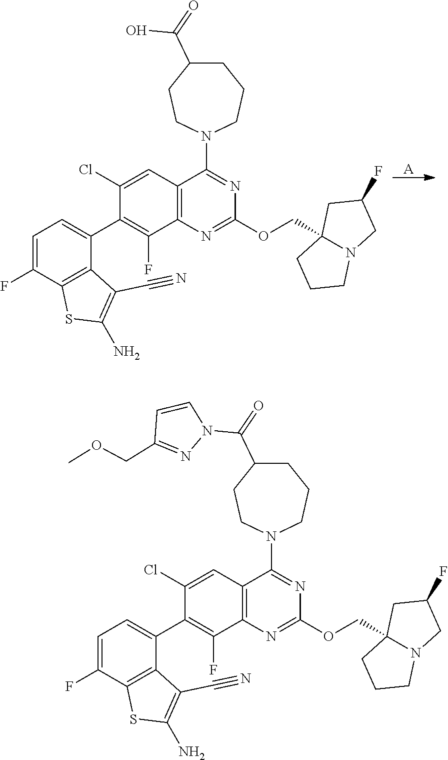

Step A. 2-amino-4-(6-chloro-8-fluoro-2-(((2R,7aS)-2-fluorotetrahydro-1H-pyrrolizin-7a(5H)-yl)methoxy)-4-(4-(3-(methoxymethyl)-1H-pyrazole-1-carbonyl) azepan-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (132 mg, 1.0 equiv) and 3-(methoxymethyl)-1H-pyrazole (55.1 mg, 2.5 equiv) in DMAc (1 mL) were added HATU (112 mg, 1.5 equiv) and DIEA (76.3 mg, 3.0 equiv). The reaction was stirred at 25 °C for 14 hours. The mixture was purified with prep-HPLC [column: Waters Xbridge 150×25 mm×10 μm; mobile phase: water (NH₄HCO₃)-acetonitrile; gradient: 65%-85% B over 8 minutes] to afford the title compound (13.1 mg, 8% yield) as white solid; ¹H NMR (400 MHz, DMSO-d₆) δ=8.36 (d, J=2.8 Hz, 1H), 8.10 (s, 2H), 8.01-7.95 (m, 1H), 7.29-7.19 (m, 1H), 7.18-7.11 (m, 1H), 6.60 (dd, J=1.2, 2.8 Hz, 1H), 5.36-5.17 (m, 1H), 4.42 (d, J=54.4 Hz, 2H), 4.28-4.13 (m, 2H), 4.13-3.98 (m, 2H), 3.98-3.71 (m, 3H), 3.29 (d, J=2.0 Hz, 3H), 3.14-3.04 (m, 2H), 3.04-2.97 (m, 1H), 2.87-2.77 (m, 1H), 2.36-2.25 (m, 1H), 2.22-1.97 (m, 7H), 1.88-1.61 (m, 4H); LCMS (ESI, M+1): m/z=765.3.

### Example 191

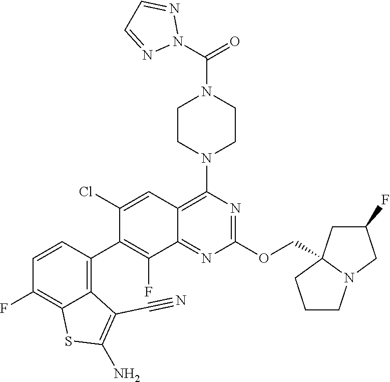 [SMILES: N#Cc1c(N)sc2c(F)ccc(-c3c(Cl)cc4c(N5CCN(C(=O)n6nccn6)CC5)nc(OC[C@@]56CCCN5C[C@H](F)C6)nc4c3F)c12]

### 4-(4-(4-(2H-1,2,3-triazole-2-carbonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

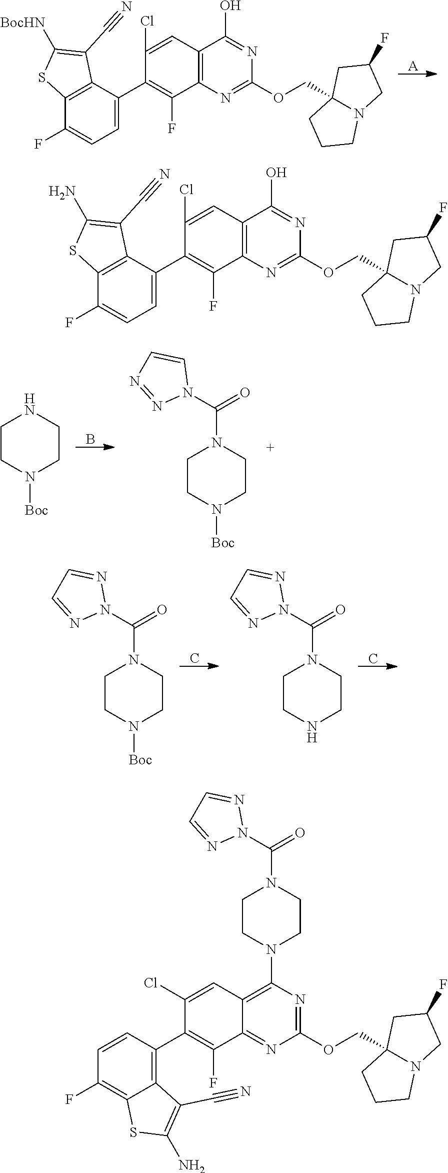

Step A. 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (200 mg, 1.0 equiv) in DCM (2 mL) was added TFA (2 mL, 87.0 equiv). The reaction was stirred at 25 °C for 15 minutes. The mixture was concentrated, basified with saturated NaHCO₃ solution (10 mL) and extracted with ethyl acetate (3×15 mL). The combined organic layers were dried over anhydrous sodium sulfate, concentrated to afford the title compound (133 mg, crude) as yellow solid.

Step B. tert-butyl 4-(2H-1,2,3-triazole-2-carbonyl)piperazine-1-carboxylate: To a solution of 1H-1,2,3-triazole (1.48 g, 2.0 equiv) in DCM (10 mL) were added bis(trichloromethyl) carbonate (956 mg, 0.3 equiv) and TEA (2.17 g, 2.0 equiv). The reaction was stirred at 0 °C for 1 hour. tert-butyl piperazine-1-carboxylate (2 g, 1.0 equiv) and TEA (2.17 g, 2.0 equiv) in DCM (10 mL) were added to the mixture. The reaction was stirred at 25 °C for 1 hour. The mixture was washed with HCl (2M in H₂O, 20 mL) and NaHCO₃ solution (30 mL). The organic layer was dried over anhydrous sodium sulfate, concentrated and purified with SFC separation [column: DAICEL CHIRALPAK IC, 250×30 mm, 10 μm; A: CO₂, B: MeOH (0.1% NH₃H₂O), B %: 30% B isocratic elution mode] to afford to afford two isomers.

Isomer 1: tert-butyl 4-(1H-1,2,3-triazole-1-carbonyl)piperazine-1-carboxylate (720 mg, 22% yield) as white solid; LCMS (ESI, M+1): m/z=304.1; SFC>99% ee, column: Chiralpak IC-3 50×4.6 mm I.D., 3 μm; mobile phase: 20% to 60% MeOH (0.05% DEA) in CO₂, flow rate: 3 mL/min, detector: PDA, t_{R}: 0.613 min.

Isomer 2: tert-butyl 4-(2H-1,2,3-triazole-2-carbonyl)piperazine-1-carboxylate (1.3 g, 42% yield) as white solid; LCMS (ESI, M+23): m/z=304.1; SFC=98% ee, column: Chiralpak IC-3 50×4.6 mm I.D., 3 μm; mobile phase: 20% to 60% MeOH (0.05% DEA) in CO₂, flow rate: 3 mL/min, detector: PDA, t_{R}: 0.819 min.

Step C. piperazin-1-yl(2H-1,2,3-triazol-2-yl)methanone: To a solution of tert-butyl 4-(2H-1,2,3-triazole-2-carbonyl)piperazine-1-carboxylate (50 mg, 1.0 equiv) in MeOH (0.2 mL) was added HCl·dioxane (2 M, 0.5 mL). The reaction was stirred at 25 °C for 0.5 hours. The mixture was concentrated to afford the title compound (29.9 mg, crude, HCl salt) as white solid.

Step D. 4-(4-(4-(2H-1,2,3-triazole-2-carbonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (50 mg, 1.0 equiv) and piperazin-1-yl(2H-1,2,3-triazol-2-yl)methanone (29.9 mg, 1.5 equiv, HCl salt) in DMF (0.5 mL) were added PyBOP (71.5 mg, 1.5 equiv) and DIEA (118 mg, 10 equiv) at 0 °C. The mixture was stirred at 25 °C for 0.5 hours. The mixture was filtered and the filtrate was purified by prep-HPLC [Waters Xbridge 150×25 mm×5 μm; A: water (ammonia hydroxide), B: ACN, B %: 35%-65% over 10 min] to afford the title compound (17.4 mg, 26% yield) as off-white solid; ¹H NMR (400 MHz, METHANOL-d₄) δ=8.01 (s, 2H), 7.96 (d, J=1.2 Hz, 1H), 7.21 (dd, J=5.2, 8.4 Hz, 1H), 7.04 (t, J=8.8 Hz, 1H), 5.39-5.21 (m, 1H), 4.33-4.22 (m, 2H), 4.18-3.97 (m, 8H), 3.28-3.12 (m, 3H), 3.07-2.98 (m, 1H), 2.38-2.12 (m, 3H), 2.05-1.84 (m, 3H); LCMS (ESI, M+1): m/z=709.2.

### Example 192

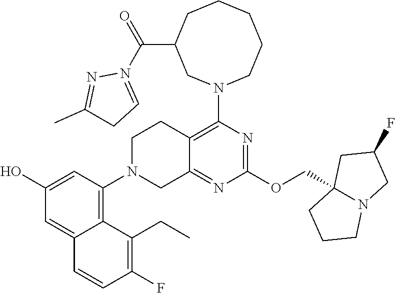

(1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azocan-3-yl)(3-methyl-1H-pyrazol-1-yl)methanone

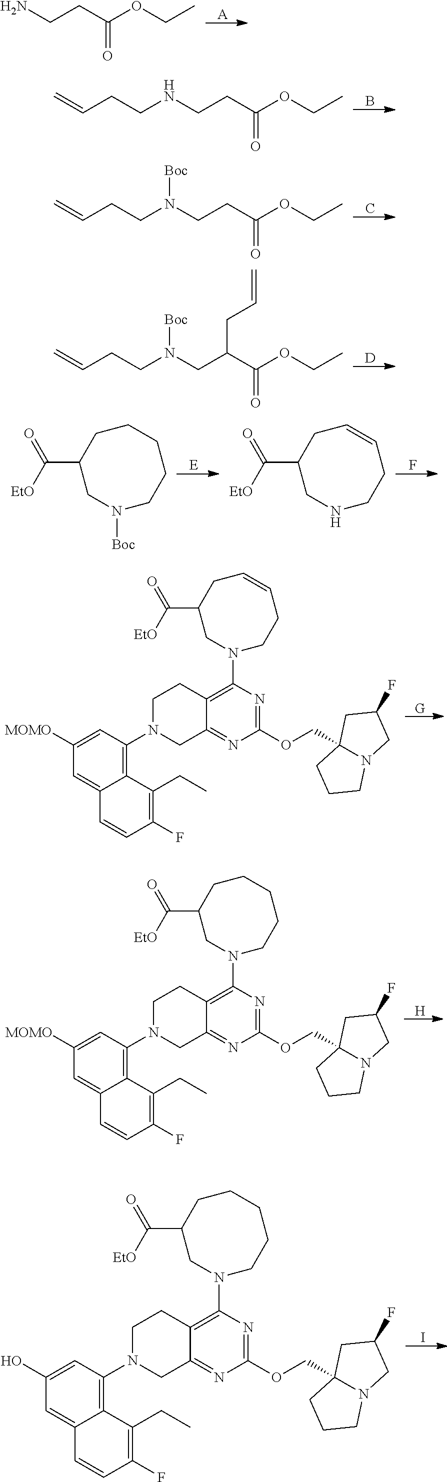

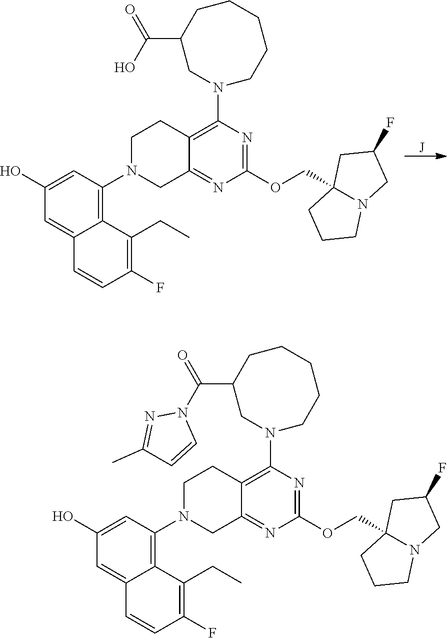

Step A. ethyl 3-(but-3-en-1-ylamino)propanoate: To a solution of ethyl ethyl 3-aminopropanoate (49.5 g, 1.5 equiv, HCl) in ACN (1000 mL) was added K₂CO₃ (87.5 g, 3.0 equiv). The reaction was stirred at 25 °C for 1 hour. 4-bromobut-1-ene (28.5 g, 1.0 equiv) was added. The reaction was stirred at 45 °C for 17 hours. The reaction was concentrated to afford the title compound (48.3 g, crude) as colorless oil; ¹H NMR (400 MHz, CHLOROFORM-d) δ=5.91-5.64 (m, 1H), 5.16-4.89 (m, 1H), 4.21-3.97 (m, 2H), 3.00-2.75 (m, 2H), 2.66 (t, J=6.8 Hz, 1H), 2.56-2.34 (m, 2H), 2.29-2.09 (m, 1H), 1.34-1.14 (m, 3H).

Step B. ethyl 3-(but-3-en-1-yl(tert-butoxycarbonyl)amino)propanoate: To a solution of ethyl 3-(but-3-enylamino)propanoate (43.3 g, 1.0 equiv) in DCM (450 mL) was added TEA (51.1 g, 2.0 equiv) and Boc₂O (82.7 g, 1.5 equiv). The reaction was stirred at 25 °C for 1 hour. The mixture was concentrated and purified by column chromatography (SiO₂, PE/EA=1/0 to 20:1) to afford the title compound (40.2 g, 59% yield) as yellow oil.

Step C. ethyl 2-((but-3-en-1-yl(tert-butoxycarbonyl)amino)methyl)pent-4-enoate: To a solution of ethyl 3-(but-3-en-1-yl(tert-butoxycarbonyl)amino)propanoate (20.0 g, 1.0 equiv) in THF (200 mL), the reaction was degassed and purged with nitrogen 3 times. LDA (2 M, 1.5 equiv) was added slowly at −65 °C, the reaction was stirred at −40 °C for 1 hour. 3-iodoprop-1-ene (13.6 g, 1.1 equiv) was added at −65 °C. The reaction was stirred at −40 °C for 0.5 hours. The mixture was quenched with water (100 mL) at 0 °C and extracted with ethyl acetate (3×100 mL). The combined organic layer was dried over anhydrous Na₂SO₄, filtered, concentrated and purified by column chromatography (SiO₂, PE/EA=1/0 to 20:1) to afford the title compound (3.90 g, 15% yield) as yellow oil; ¹H NMR (400 MHz, CHLOROFORM-d) δ=5.81-5.64 (m, 2H), 5.11-4.95 (m, 4H), 4.16-4.06 (m, 2H), 3.50-3.21 (m, 3H), 3.17-3.05 (m, 1H), 2.95-2.68 (m, 1H), 2.39-2.11 (m, 4H), 1.44 (s, 9H), 1.23 (t, J=7.2 Hz, 3H).

Step D. 1-(tert-butyl) 3-ethyl(Z)-3,4,7,8-tetrahydroazocine-1,3 (2H)-dicarboxylate: To a solution of ethyl 2-((but-3-en-1-yl(tert-butoxycarbonyl)amino)methyl)pent-4-enoate (1.00 g, 1.0 equiv) in DCM (150 mL) was added Grubbs catalyst (265 mg, 0.10 equiv). The reaction was purged with nitrogen 3 times. The reaction was stirred at 40 °C for 1.5 hours. The mixture was filtered, concentrated and purified by column chromatography (SiO₂, PE/EA=1/0 to 20/1) to afford the title compound (650 mg, 71% yield) as brown oil; ¹H NMR (400 MHz, CHLOROFORM-d) δ=5.96-5.59 (m, 2H), 4.20-4.06 (m, 3H), 3.91-3.75 (m, 1H), 3.42-3.05 (m, 1H), 3.01-2.89 (m, 1H), 2.83-2.58 (m, 1H), 2.49-2.21 (m, 3H), 2.19-2.06 (m, 1H), 1.47 (d, J=3.2 Hz, 9H), 1.26 (td, J=7.2, 10.3 Hz, 3H).

Step E. ethyl(Z)-1,2,3,4,7,8-hexahydroazocine-3-carboxylate: A solution of 1-(tert-butyl) 3-ethyl(Z)-3,4,7,8-tetrahydroazocine-1,3 (2H)-dicarboxylate (700 mg, 1.0 equiv) in HCl·MeOH (2 M, 14.0 mL) was stirred at 25 °C for 1 hour. The mixture was concentrated, diluted with MeOH (2 mL), adjusted to pH=7 using NaHCO₃solid, filtered and concentrated to afford the title compound ethyl (5Z)-1,2,3,4,7,8-hexahydroazocine-3-carboxylate (450 mg, crude) was obtained as a yellow solid.

Step F. ethyl(Z)-1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,2,3,4,7,8-hexahydroazocine-3-carboxylate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-ol (200 mg, 1.0 equiv) and ethyl(Z)-1,2,3,4,7,8-hexahydroazocine-3-carboxylate (158 mg, 3.0 equiv) in DMF (2 mL) were added 4 Å molecular sieve (50 mg) and K₃PO₄ (183 mg, 3.0 equiv). The reaction was stirred at 80 °C for 2 hours. The mixture was filtered and purified by prep-HPLC [Waters Xbridge Prep OBD C18 150×40 mm×10 μm; A: water (NH₄HCO₃); B: ACN, B %: 70%-100% over 20 min] to afford the title compound (160 mg, 79% yield) as a yellow solid; LCMS (ESI, M+1): m/z=706.4.

Step G. ethyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-3-carboxylate: To a mixture of Pd/C (20.0 mg, 10% purity, 0.1 equiv) in MeOH (3 mL) was added ethyl(Z)-1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)-1,2,3,4,7,8-hexahydroazocine-3-carboxylate (100 mg, 1 equiv) under N₂. The reaction was degassed and purged with H₂ 3 times. The reaction was stirred under H₂ (15 psi) at 25 °C for 1 hour. The mixture was filtered and concentrated to afford the title compound; LCMS (ESI, M+1): m/z=708.3.

Step H. ethyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-3-carboxylate: A solution of ethyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-3-carboxylate (100 mg, 1.0 equiv) in HCl·MeOH (2 M, 2 mL) was stirred at 25 °C for 1 hour. The mixture was concentrated, diluted with MeOH (3 mL), adjusted to pH=7 using NaHCO₃solid, filtered and concentrated to afford the title compound (130 mg, crude) as a yellow solid.

Step I. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-3-carboxylic acid: To a solution of ethyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-3-carboxylate (120 mg, 1.0 equiv) in MeOH (1 mL) was added LiOH·H₂O (2 M, 1 mL, 16 equiv). The reaction was stirred at 25 °C for 0.5 hours. The mixture was filtered, concentrated and purified by reversed-phase flash (0.1% FA condition) and prep-HPLC [C18 150×25 mm×10 μm; A: water (FA), B: FA, B %: 20%-50% over 1 min] to afford the title compound (30.0 mg, 24%, HCOOH) as white solid. LCMS (ESI, M+1): m/z=636.4.

Step J. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) azocan-3-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)azocane-3-carboxylic acid (20.0 mg, 1.0 equiv, HCOOH) and 3-methyl-1H-pyrazole (7.23 mg, 3.0 equiv) in DMF (0.5 mL) were added DIEA (19.0 mg, 5.0 equiv) and HATU (22.3 mg, 2.0 equiv). The reaction was stirred at 25 °C for 2 hours. The mixture was filtered and purified by prep-HPLC [Waters Xbridge 150×25 mm×5 μm; A: water (NH₄HCO₃), B: ACN, B %: 52%-82% over 15 min] to afford the title compound (8.01 mg, 38%) as yellow solid; ¹H NMR (400 MHz, CHLOROFORM-d) δ=1.08 (br t, J=6.92 Hz, 3H), 1.57-1.71 (m, 3H), 1.75-1.96 (m, 8H), 1.99-2.04 (m, 1H), 2.09-2.25 (m, 2H), 2.31 (br dd, J=3.64, 1.76 Hz, 3H), 2.58-2.74 (m, 5H), 2.87-3.00 (m, 2H), 3.05-3.15 (m, 2H), 3.27-3.42 (m, 5H), 3.69-3.85 (m, 2H), 4.18-4.60 (m, 3H), 5.10-5.38 (m, 1H), 6.21-6.30 (m, 1H), 6.85-7.01 (m, 2H), 7.10 (t, J=9.32 Hz, 1H), 7.34-7.45 (m, 1H), 8.16 (d, J=2.52 Hz, 1H); LCMS (ESI, M+1): m/z=700.4.

### Example 193

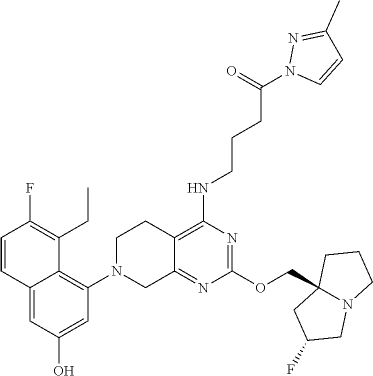 [SMILES: CCc1c(F)ccc2cc(O)cc(N3CCc4c(nc(OC[C@@]56CCCN5C[C@H](F)C6)nc4NCCCC(=O)n4ccc(C)n4)C3)c12]

### 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)-1-(3-methyl-1H-pyrazol-1-yl) butan-1-one

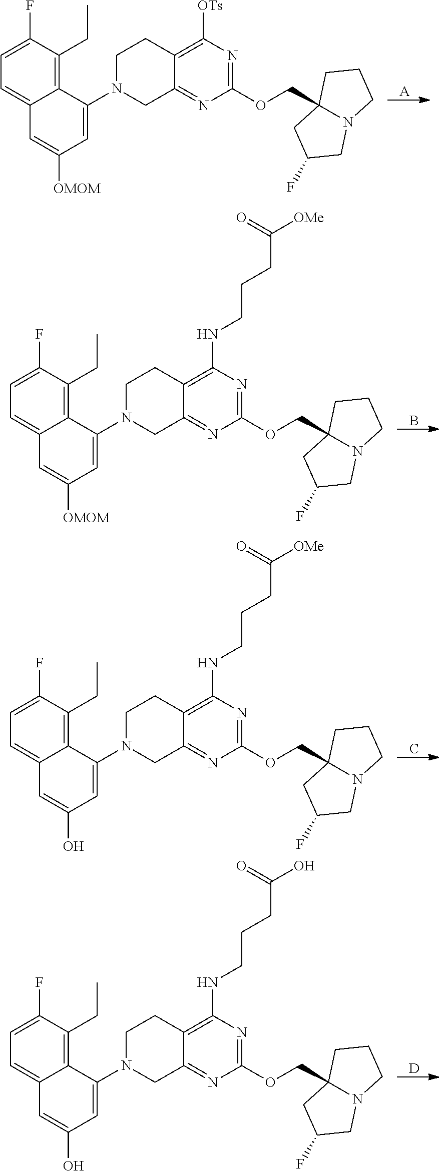

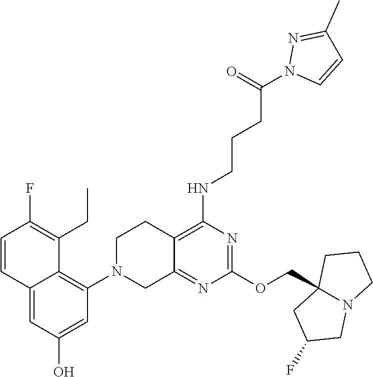 [SMILES: CCc1c(F)ccc2cc(O)cc(N3CCc4c(nc(OC[C@@]56CCCN5C[C@H](F)C6)nc4NCCCC(=O)n4ccc(C)n4)C3)c12]

Step A. methyl 4-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)butanoate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (300 mg, 1.0 equiv) in DMF (7 mL) were added methyl 4-aminobutanoate (99.5 mg, 1.5 equiv, HCl), K₃PO₄ (275 mg, 3.0 equiv) and 4 Å molecular sieve (10 mg). The reaction was stirred at 80 °C for 2 hours. The mixture was filtered and diluted with water (30 mL) and extracted with ethyl acetate (20 mL×3). The combined organic layers were washed with brine (30 mL), dried over anhydrous sodium sulfate, concentrated and purified with prep-TLC (SiO₂, petroleum ether/ethyl acetate=0/1) to afford the title compound (130 mg, 47% yield) as yellow oil; LCMS (ESI, M+1): m/z=640.3.

Step B. methyl 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)butanoate: To a solution of methyl 4-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)butanoate (110 mg, 1.0 equiv) in MeCN (4 mL) was added HCl·dioxane (4 mL). The reaction was stirred at 0 °C for 1 hour. The mixture was concentrated under reduced pressure to remove solvent to afford the title compound (100 mg, crude) as brown solid; LCMS (ESI, M+1): m/z=596.3.

Step C. 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)butanoic acid: To a solution of methyl 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)butanoate (100 mg, 1.0 equiv) in MeOH (1.2 mL) was added LiOH (0.42 mL, 2M). The mixture was stirred at 25 °C for 1 hour. The mixture was purified with prep-HPLC [FA condition; column: Phenomenex luna C18 150×40 mm×15 μm; mobile phase: water (FA)-ACN; gradient: 12%-42% B over 15 min] to afford the title compound (50.0 mg, 51% yield) as off-white solid; LCMS (ESI, M+1): m/z=582.3.

Step D. 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)-1-(3-methyl-1H-pyrazol-1-yl) butan-1-one: To a solution of 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)butanoic acid (20.0 mg, 1.0 equiv) in DMF (1 mL) were added 3-methyl-1H-pyrazole (4.23 mg, 1.5 equiv), DIEA (22.2 mg, 5.0 equiv) and HATU (19.6 mg, 1.5 equiv). The reaction was stirred at 25 °C for 1 hour. The mixture was diluted with water (10 mL) and extracted with ethyl acetate (10 mL×2). The combined organic layers were washed with brine (10 mL), dried over anhydrous sodium sulfate, concentrated and purified with prep-HPLC [neutral condition; column: Waters Xbridge Prep OBD C18 150×40 mm×10 μm; mobile phase: water (NH₄HCO₃)-ACN; gradient: 43%-73% B over 20 min] to afford the title compound (14.2 mg, 62% yield) as yellow solid; ¹H NMR (400 MHz, CHLOROFORM-d) δ=8.16 (d, J=2.4 Hz, 1H), 7.41 (dd, J=6.0, 8.8 Hz, 1H), 7.10 (dt, J=2.4, 9.2 Hz, 1H), 6.91 (s, 2H), 6.27 (d, J=2.8 Hz, 1H), 5.35-5.12 (m, 2H), 4.19-4.04 (m, 2H), 3.96 (br d, J=16.8 Hz, 1H), 3.74-3.54 (m, 3H), 3.46-3.36 (m, 1H), 3.32-3.11 (m, 8H), 3.00-2.92 (m, 1H), 2.68-2.54 (m, 1H), 2.32 (s, 3H), 2.29-2.10 (m, 6H), 1.96-1.85 (m, 3H), 1.03 (dt, J=2.8, 7.2 Hz, 3H); LCMS (ESI, M+1): m/z=646.4.

### Example 194

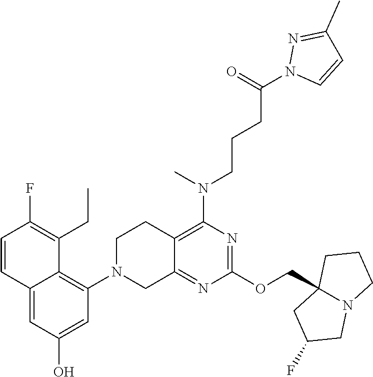 [SMILES: CCc1c(F)ccc2cc(O)cc(N3CCc4c(nc(OC[C@@]56CCCN5C[C@H](F)C6)nc4N(C)CCCC(=O)n4ccc(C)n4)C3)c12]

### 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)-1-(3-methyl-1H-pyrazol-1-yl) butan-1-one

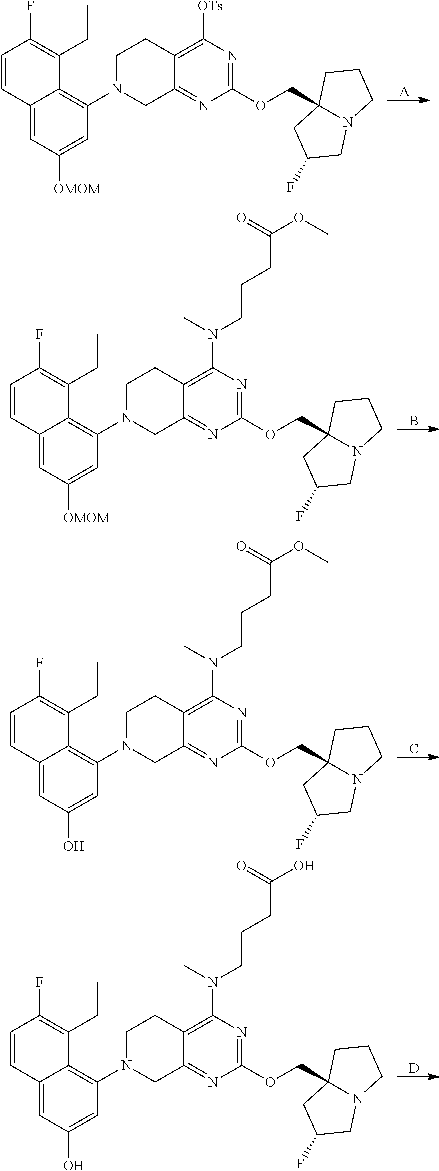

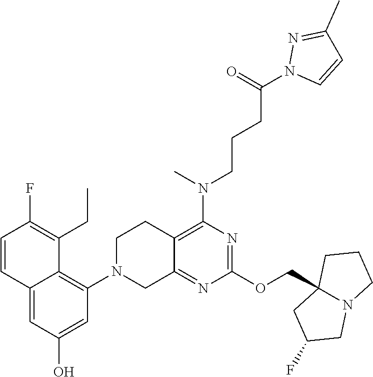 [SMILES: CCc1c(F)ccc2cc(O)cc(N3CCc4c(nc(OC[C@@]56CCCN5C[C@H](F)C6)nc4N(C)CCCC(=O)n4ccc(C)n4)C3)c12]

Step A. methyl 4-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)butanoate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (500 mg, 1.0 equiv) in DMF (8 mL) were added K₃PO₄ (458 mg, 3.0 equiv), 4 Å molecular sieve (50 mg) and methyl 4-(methylamino)butanoate (181 mg, 1.5 equiv, HCl). The reaction was stirred at 80 °C for 2 hours. The mixture was diluted with water (30 mL) and extracted with ethyl acetate (20 mL×3). The combined organic layers were washed with brine (30 mL), dried over anhydrous sodium sulfate, concentrated and purified with column chromatography [SiO₂, petroleum ether/ethyl acetate=1/1 to ethyl acetate/methanol=10/1] to afford the title compound (320 mg, 65% yield) as brown solid; LCMS (ESI, M+1): m/z=654.5.

Step B. methyl 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)butanoate: To a solution of methyl 4-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)butanoate (300 mg, 1.0 equiv) in MeOH (3 mL) was added HCl·MeOH (3 mL, 2M). The reaction was stirred at 0 °C for 1 hour. The mixture was concentrated under reduced pressure to afford the title compound (250 mg, crude) as brown solid; LCMS (ESI, M+1): m/z=610.4.

Step C. 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)butanoic acid: To a solution of methyl 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)butanoate (240 mg, 1.0 equiv) in MeOH (2.4 mL) was added LiOH (0.8 mL, 2M). The reaction was stirred at 20 °C for 2 hours. The mixture was purified with prep-HPLC [column: Waters Xbridge 150×25 mm×5 μm; mobile phase: [water (ammonia hydroxide v/v)-ACN]; gradient: 0%-30% B over 10 min] to afford the title compound (80.0 mg, 34% yield) as white solid; LCMS (ESI, M+1): m/z=596.3

Step D. 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)-1-(3-methyl-1H-pyrazol-1-yl) butan-1-one: To a solution of 4-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)butanoic acid (70.0 mg, 1.0 equiv) in DMF (1 mL) were added HATU (67.0 mg, 1.5 equiv), 3-methyl-1H-pyrazole (14.5 mg, 1.5 equiv) and DIEA (75.9 mg, 5.0 equiv). The reaction was stirred at 25 °C for 1 hour. The mixture was purified with prep-HPLC [column: Waters Xbridge Prep OBD C18 150×40 mm×10 μm; mobile phase: [water (NH₄HCO₃)-ACN]; gradient: 50%-80% B over 20 min] to afford the title compound (29.9 mg, 35% yield) as off-white solid; ¹H NMR (400 MHz, CHLOROFORM-d) δ=8.16-8.07 (m, 1H), 7.41 (dd, J=6.0, 8.8 Hz, 1H), 7.11 (dt, J=3.2, 9.2 Hz, 1H), 6.96-6.90 (m, 2H), 6.25 (d, J=2.8 Hz, 1H), 5.34-5.14 (m, 1H), 4.16-4.02 (m, 3H), 3.73-3.54 (m, 3H), 3.42-3.21 (m, 5H), 3.13 (d, J=6.4 Hz, 3H), 3.10-2.86 (m, 5H), 2.60-2.51 (m, 1H), 2.31 (s, 3H), 2.27-1.99 (m, 6H), 1.97-1.86 (m, 3H), 1.10 (t, J=7.2 Hz, 3H); LCMS (ESI, M+1): m/z=660.4.

### Example 195

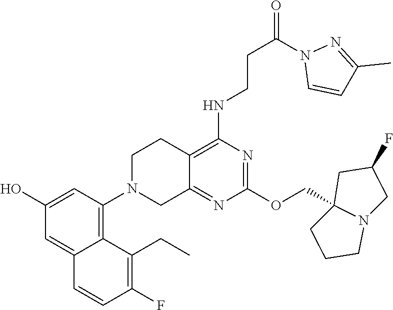 [SMILES: CCc1c(F)ccc2cc(O)cc(N3CCc4c(nc(OC[C@@]56CCCN5C[C@H](F)C6)nc4NCCC(=O)n4ccc(C)n4)C3)c12]

### 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)-1-(3-methyl-1H-pyrazol-1-yl) propan-1-one

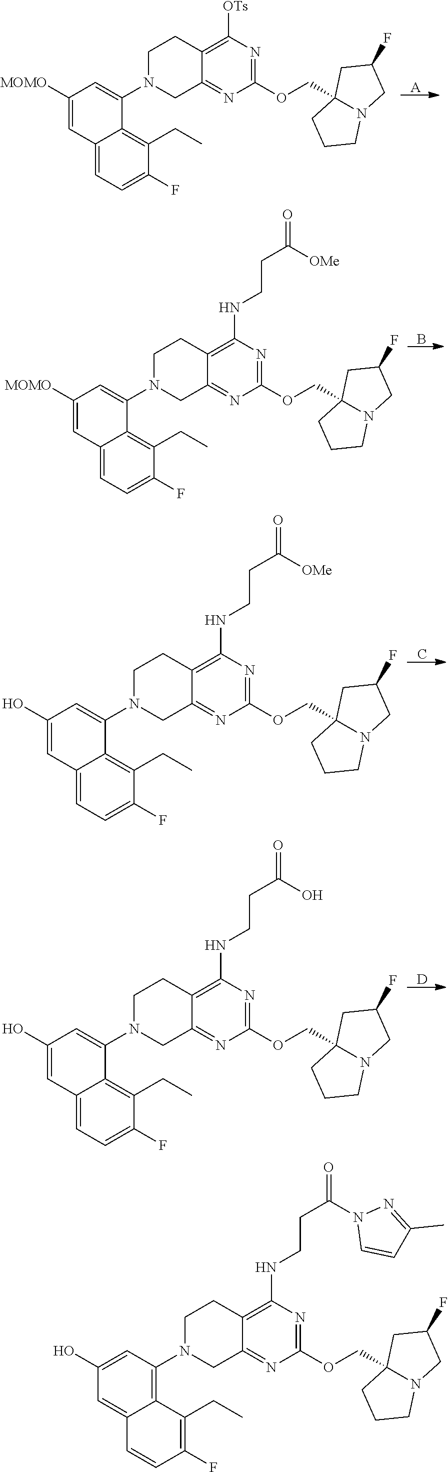

Step A. methyl 3-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)propanoate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (400 mg, 1.0 equiv) in DMF (4 mL) were added K₃PO₄ (367 mg, 3.0 equiv), methyl 3-aminopropanoate (120 mg, 1.5 equiv) and 4 Å molecular sieve (40 mg). The reaction was stirred at 80 °C for 2 hours. The mixture was diluted with water (20 mL) and extracted with ethyl acetate (20 mL×3). The combined organic layers were washed with brine (20 mL), dried over anhydrous sodium sulfate and purified with prep-TLC [SiO₂, petroleum ether:ethyl acetate=0:1] to afford the title compound (360 mg, 51% yield) as yellow oil; LCMS (ESI, M+1): m/z=626.4.

Step B. methyl 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)propanoate: To a solution of methyl 3-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)propanoate (300 mg, 1.0 equiv) in MeOH (1.5 mL) was added HCl·MeOH (1.5 mL, 2M). The reaction was stirred at 25 °C for 1 hour. The mixture was concentrated to afford the title compound (270 mg, crude) as yellow oil; LCMS (ESI, M+1): m/z=582.3.

Step C. 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino) propanoic acid: To a solution of methyl 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)propanoate (270 mg, 1.0 equiv) in MeOH (1 mL) was added LiOH·H₂O (2.0 mL, 2M). The reaction was stirred at 25 °C for 1 hour. The mixture was filtered and purified with prep-HPLC [column: Phenomenex luna C18 150×40 mm×15 μm; mobile phase: [water (FA)-ACN]; gradient: 10%-40% B over 15 min] to afford the title compound (70.0 mg, 26% yield) as off-white solid; LCMS (ESI, M+1): m/z=568.3.

Step D. 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino)-1-(3-methyl-1H-pyrazol-1-yl) propan-1-one: To a solution of 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)amino) propanoic acid (40.0 mg, 1.0 equiv) in DMF (0.5 mL) were added DIEA (45.5 mg, 5.0 equiv), 3-methyl-1H-pyrazole (8.68 mg, 1.5 equiv) and HATU (40.2 mg, 1.5 equiv). The reaction was stirred at 25 °C for 0.5 hours. The mixture was filtered and purified with prep-HPLC [column: Waters Xbridge Prep OBD C18 150×40 mm×10 μm; mobile phase: [water (NH₄HCO₃)-ACN]; gradient: 40%-70% B over 20 min] to afford the title compound (6.15 mg, 13% yield) as yellow solid; ¹H NMR (400 MHz, CHLOROFORM-d) δ=8.16 (d, J=2.4 Hz, 1H), 7.45-7.41 (m, 1H), 7.15-7.10 (m, 1H), 6.92-6.91 (m, 2H), 6.29 (d, J=2.4 Hz, 1H), 5.46-5.43 (m, 1H), 5.33-5.20 (m, 1H), 4.17-4.12 (m, 1H), 4.09-4.06 (m, 1H), 4.00-3.97 (m, 3H), 3.68 (br dd, J=7.2, 16.8 Hz, 1H), 3.46-3.41 (m, 3H), 3.29-3.27 (m, 3H), 3.17 (br s, 2H), 3.00-2.94 (m, 1H), 2.67-2.58 (m, 1H), 2.33 (s, 3H), 2.30-2.24 (m, 2H), 2.20 (br s, 1H), 2.14 (br dd, J=3.2, 7.2 Hz, 1H), 1.96-1.87 (m, 4H), 1.04-1.01 (m, 3H); LCMS (ESI, M+1): m/z=632.4.

### Example 196

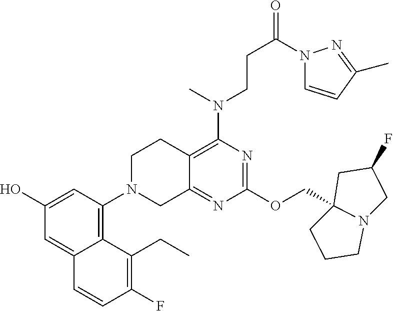 [SMILES: CCc1c(F)ccc2cc(O)cc(N3CCc4c(nc(OC[C@@]56CCCN5C[C@H](F)C6)nc4N(C)CCC(=O)n4ccc(C)n4)C3)c12]

### 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)-1-(3-methyl-1H-pyrazol-1-yl) propan-1-one

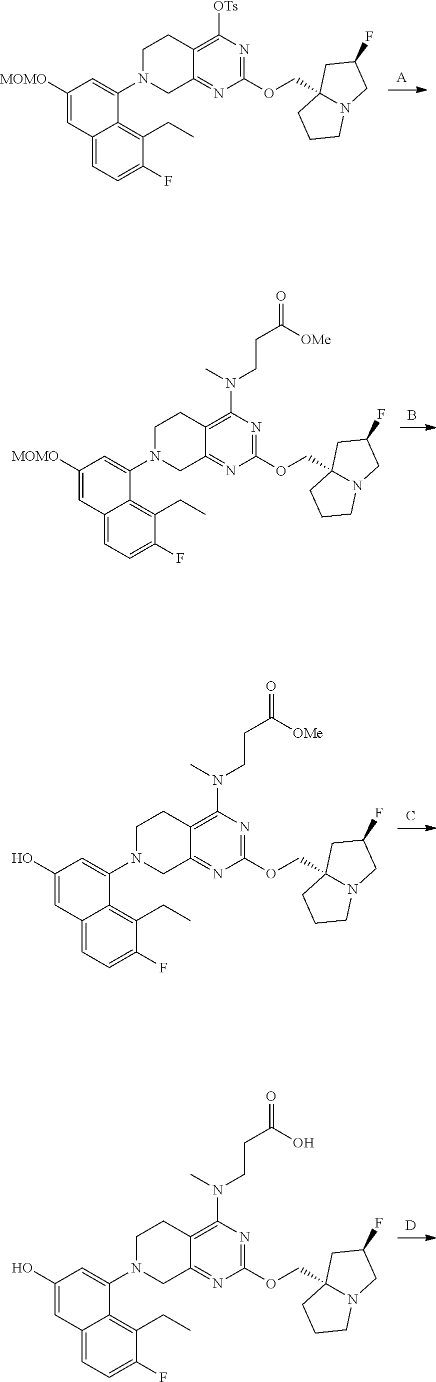

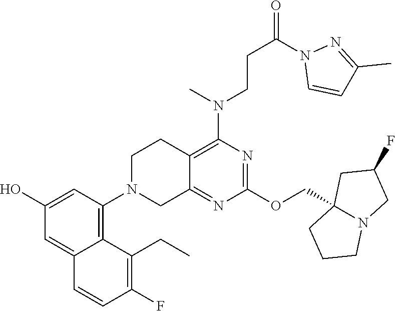 [SMILES: CCc1c(F)ccc2cc(O)cc(N3CCc4c(nc(OC[C@@]56CCCN5C[C@H](F)C6)nc4N(C)CCC(=O)n4ccc(C)n4)C3)c12]

Step A. methyl 3-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)propanoate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (500 mg, 1.0 equiv) in DMF (5 mL) were added K₃PO₄ (458 mg, 3.0 equiv), methyl 3-aminopropanoate (126 mg, 1.5 equiv) and 4 Å molecular sieve (50 mg). The reaction was stirred at 80 °C for 1 hour. The mixture was diluted with water (20 mL) and extracted with ethyl acetate (20 mL×3). The combined organic layers were washed with brine (20 mL), dried over anhydrous sodium sulfate and purified with column chromatography [SiO₂, petroleum ether/ethyl acetate=10/1 to dichloromethane/methanol=10/1] to afford the title compound (164 mg, 22% yield) as yellow solid; LCMS (ESI, M+1): m/z=640.4.

Step B. methyl 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)propanoate: To a solution of methyl 3-((7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)propanoate (164 mg, 1.0 equiv) in MeOH (0.8 mL) was added HCl·MeOH (0.8 mL, 2M). The reaction was stirred at 25 °C for 0.5 hours. The mixture was concentrated to afford the title compound (140 mg, crude) as brown oil; LCMS (ESI, M+1): m/z=596.3.

Step C. 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino) propanoic acid: To a solution of methyl 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)propanoate (140 mg, 1.0 equiv) in MeOH (0.9 mL) was added LiOH·H₂O (0.3 mL, 2M). The reaction was stirred at 25 °C for 0.5 hours. The mixture was filtered and purified with prep-HPLC [column: Phenomenex luna C18 150×40 mm×15 μm; mobile phase: [water (FA)-ACN]; gradient: 15%-45% B over 15 min] to afford the title compound (38.0 mg, 27% yield) as yellow solid; LCMS (ESI, M+1): m/z=582.5.

Step D. 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino)-1-(3-methyl-1H-pyrazol-1-yl) propan-1-one: To a solution f 3-((7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl)(methyl)amino) propanoic acid (38.0 mg, 1.0 equiv) in DMF (0.5 mL) were added DIEA (42.2 mg, 5.0 equiv), 3-methyl-1H-pyrazole (8.05 mg, 1.5 equiv) and HATU (37.3 mg, 1.5 equiv). The reaction was stirred at 25 °C for 0.5 hours. The mixture was filtered and purified with prep-HPLC [column: Phenomenex luna C18 150×25 mm×10 μm; mobile phase: [water (FA)-ACN]; gradient: 22%-52% B over 9 min] to afford the title compound (10.5 mg, 23% yield, 0.1HCOOH) as off-white solid; ¹H NMR (400 MHz, CHLOROFORM-d) δ=8.40 (s, 1H), 8.14 (d, J=2.8 Hz, 1H), 7.40-7.35 (m, 1H), 7.07 (br t, J=8.8 Hz, 1H), 6.99-6.91 (m, 2H), 6.26 (d, J=2.8 Hz, 1H), 5.35-5.22 (m, 1H), 4.26-3.89 (m, 6H), 3.69 (br dd, J=11.2, 17.6 Hz, 1H), 3.55-3.27 (m, 8H), 3.20 (d, J=4.0 Hz, 3H), 3.03-2.98 (m, 3H), 2.55-2.49 (m, 2H), 2.30 (s, 3H), 2.23-2.10 (m, 2H), 1.98 (br d, J=7.6 Hz, 2H), 1.10-1.06 (m, 3H); 1.25 (s, 3H); LCMS (ESI, M+1): m/z=646.4.

### Example 197

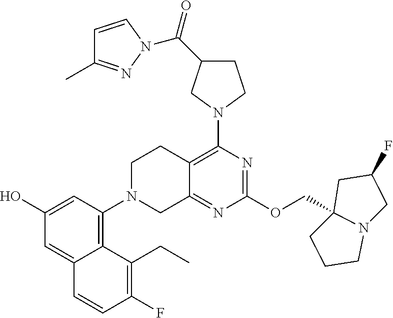 [SMILES: CCc1c(F)ccc2cc(O)cc(N3CCc4c(nc(OC[C@@]56CCCN5C[C@H](F)C6)nc4N4CCC(C(=O)n5ccc(C)n5)C4)C3)c12]

### (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidin-3-yl)(3-methyl-1H-pyrazol-1-yl)methanone

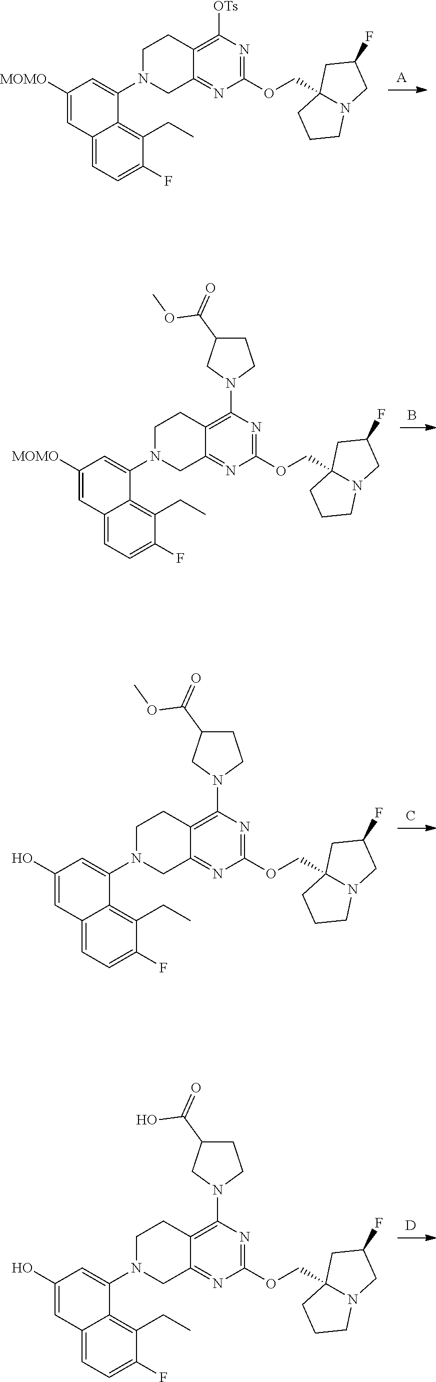

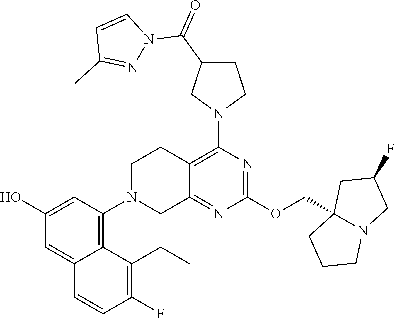 [SMILES: CCc1c(F)ccc2cc(O)cc(N3CCc4c(nc(OC[C@@]56CCCN5C[C@H](F)C6)nc4N4CCC(C(=O)n5ccc(C)n5)C4)C3)c12]

Step A. methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidine-3-carboxylate: To a solution of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl 4-methylbenzenesulfonate (1 g, 1.0 equiv) and methyl pyrrolidine-3-carboxylate (357 mg, 1.5 equiv, HCl salt) in DMF (10 mL) were added PyBOP (1.12 g, 1.5 equiv) and DIEA (1.86 g, 10 equiv). The reaction was stirred at 25 °C for 1 hour. The mixture was filtered and the filtrate was purified by reversed phase flash chromatography [C18, 0.1% formic acid condition] to afford the title compound (885 mg, 94% yield) as yellow solid; LCMS (ESI, M+1): m/z=652.3.

Step B. methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidine-3-carboxylate: To a solution of methyl 1-(7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidine-3-carboxylate (850 mg, 1.0 equiv) in MeOH (9 mL) was added HCl·MeOH (2 M, 9 mL). The reaction was stirred at 25 °C for 0.5 hours. The mixture was concentrated to afford the title compound (790 mg, crude, HCl salt) as yellow oil.

Step C. 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidine-3-carboxylic acid: To a solution of methyl 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidine-3-carboxylate (790 mg, 1.0 equiv, HCl salt) in MeOH (8 mL) was added LiOH·H₂O (2 M in H₂O, 28.3 mL, 46.1 equiv). The reaction was stirred at 25 °C for 0.5 hours. The mixture was filtered and the filtrate was purified by reversed phase flash chromatography [C18, 0.1% formic acid condition] to afford the title compound (256 mg, 65% yield) as yellow solid; LCMS (ESI, M+1): m/z=594.3.

Step D. (1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidin-3-yl)(3-methyl-1H-pyrazol-1-yl)methanone: To a solution of 1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-5,6,7,8-tetrahydropyrido[3,4-d]pyrimidin-4-yl) pyrrolidine-3-carboxylic acid (100 mg, 1.0 equiv) and 3-methyl-1H-pyrazole (69.1 mg, 5 equiv) in DMF (1 mL) were added HATU (128 mg, 2.0 equiv) and DIEA (109 mg, 5.0 equiv). The reaction was stirred at 25 °C for 15 minutes. The mixture was filtered and purified by prep-HPLC [Waters Xbridge 150×25 mm×5 μm; A: water (NH₄HCO₃), B: ACN, B %: 55%-85% over 9 min] to afford the title compound (7.08 mg, 6.20% yield) as yellow solid; ¹H NMR (400 MHz, CHLOROFORM-d) δ=8.25-8.12 (m, 1H), 7.41-7.30 (m, 1H), 7.13-7.00 (m, 1H), 6.96-6.79 (m, 2H), 6.31 (br s, 1H), 5.33-5.14 (m, 1H), 4.34 (br d, J=7.6 Hz, 1H), 4.22-3.97 (m, 5H), 3.94-3.73 (m, 2H), 3.68-3.55 (m, 1H), 3.42-3.26 (m, 4H), 3.21 (br s, 3H), 3.01-2.92 (m, 2H), 2.80-2.57 (m, 2H), 2.38-2.33 (m, 3H), 2.30-2.03 (m, 4H), 1.98-1.83 (m, 3H), 1.07 (br d, J=7.6 Hz, 3H); LCMS (ESI, M+1): m/z=658.4.

### Example 198

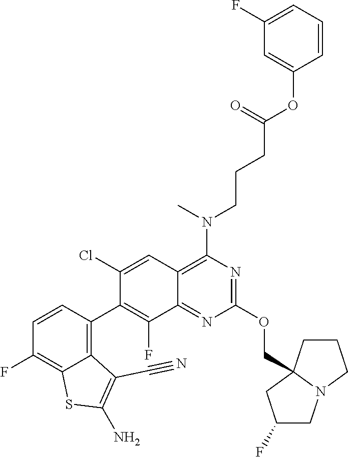 [SMILES: CN(CCCC(=O)Oc1cccc(F)c1)c1nc(OC[C@@]23CCCN2C[C@H](F)C3)nc2c(F)c(-c3ccc(F)c4sc(N)c(C#N)c34)c(Cl)cc12]

### 3-fluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate

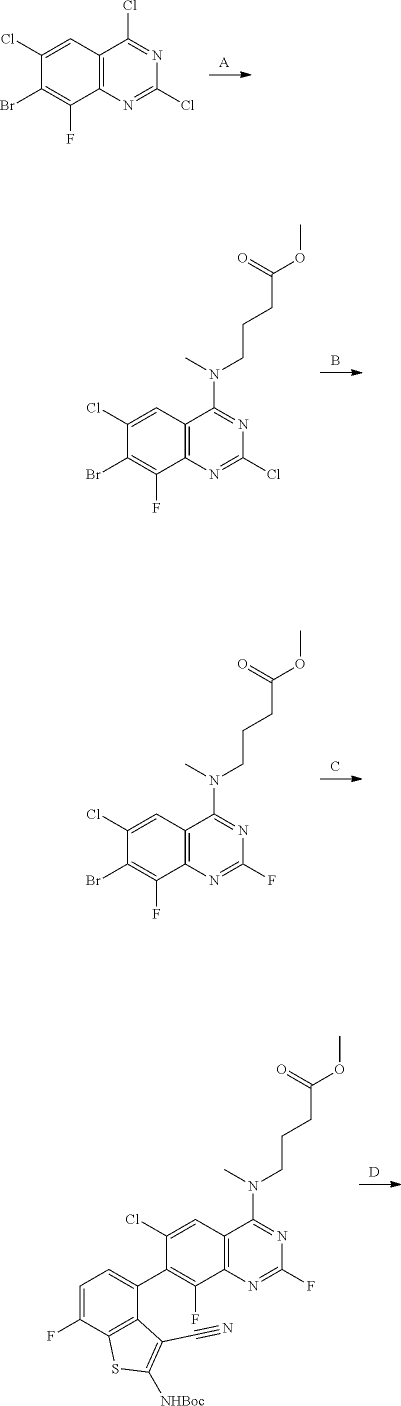

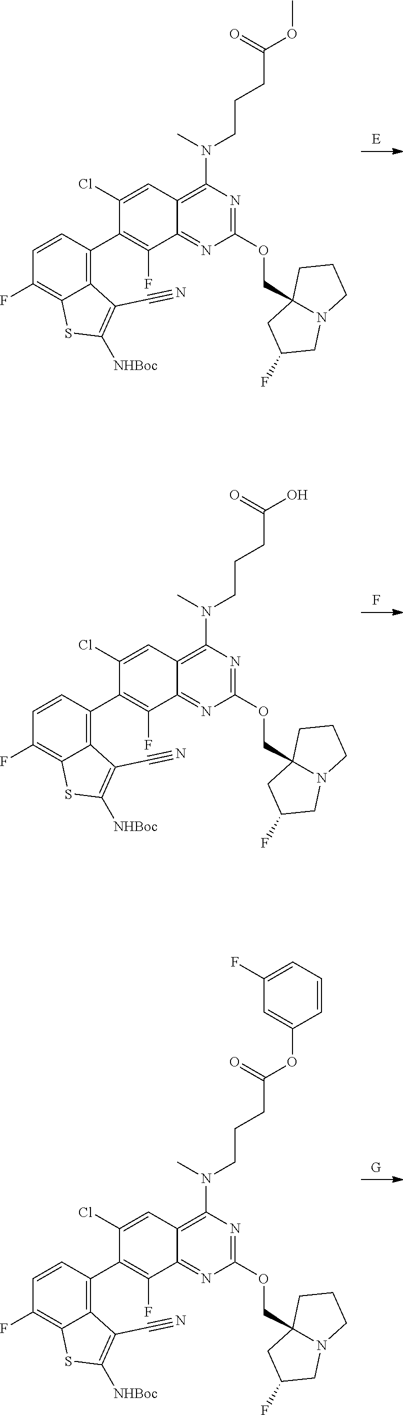

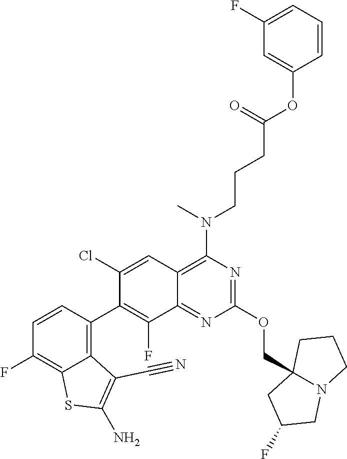 [SMILES: CN(CCCC(=O)Oc1cccc(F)c1)c1nc(OC[C@@]23CCCN2C[C@H](F)C3)nc2c(F)c(-c3ccc(F)c4sc(N)c(C#N)c34)c(Cl)cc12]

Step A. methyl 4-((7-bromo-2,6-dichloro-8-fluoroquinazolin-4-yl)(methyl)amino)butanoate: To a solution of 7-bromo-2,4,6-trichloro-8-fluoroquinazoline (2.0 g, 1.0 equiv) in DCM (30 mL) were added DIEA (3.91 g, 5.0 equiv) and methyl 4-(methylamino)butanoate;hydrochloride (1.01 g, 1.0 equiv) at −40 °C. The reaction was stirred at −40 °C for 0.5 hours. The mixture was diluted with water (20 mL) and extracted with ethyl acetate (2×10 mL). The combined organic layers were dried over anhydrous sodium sulfate, concentrated and purified with column chromatography (SiO₂, Petroleum ether/Ethyl acetate=50/1 to 3/1) to afford the title compound (2.3 g, 89% yield) as a yellow solid.

Step B. methyl 4-((7-bromo-6-chloro-2,8-difluoroquinazolin-4-yl)(methyl)amino)butanoate: To a solution of methyl 4-((7-bromo-2,6-dichloro-8-fluoroquinazolin-4-yl)(methyl)amino)butanoate (1.8 g, 1.0 equiv) in DMSO (20 mL) were added 1,4,7,10,13,16-hexaoxacyclooctadecane (112 mg, 0.10 equiv) and KF (2.46 g, 10 equiv). The reaction was stirred at 130 °C for 4 hours. The mixture diluted with water (40 mL) and extracted with ethyl acetate (2×30 mL). The combined organic layers were washed with brine (3×60 mL), dried over anhydrous sodium sulfate and concentrated to afford the title compound (1.6 g, crude) as a yellow solid.

Step C. methyl 4-((7-(2-((tert-butoxycarbonyl)amino)-3-cyano-7-fluorobenzo[b]thiophen-4-yl)-6-chloro-2,8-difluoroquinazolin-4-yl)(methyl)amino)butanoate: To a mixture of methyl 4-((7-bromo-6-chloro-2,8-difluoroquinazolin-4-yl)(methyl)amino)butanoate (1.6 g, 1.0 equiv) and tert-butyl N-[3-cyano-4-(5,5-dimethyl-1,3,2-dioxaborinan-2-yl)-7-fluoro-benzothiophen-2-yl]carbamate (2.38 g, 1.5 equiv) in THF (30 mL) were added Cs₂CO₃ (3.82 g, 3.0 equiv) and DPEPhosPdCl₂ (280 mg, 0.1.0 equiv). The reaction was degassed and purged with nitrogen 3 times. The reaction was stirred at 60 °C for 2 hours. The mixture diluted with water (30 mL) and extracted with ethyl acetate (3×25 mL). The combined organic layers were dried over anhydrous sodium sulfate, concentrated and purified by reversed phase flash [water (0.1% FA)/acetonitrile] to afford the title compound (1.67 g, 67% yield) as a yellow solid; ¹H NMR (400 MHz, CHLOROFORM-d) δ=8.05 (d, J=1.6 Hz, 1H), 7.78 (br s, 1H), 7.30 (dd, J=5.2, 8.4 Hz, 1H), 7.19-7.14 (m, 1H), 3.97-3.88 (m, 1H), 3.87-3.79 (m, 1H), 3.70 (s, 3H), 3.56 (s, 3H), 2.48 (t, J=7.2 Hz, 2H), 2.17 (quin, J=7.2 Hz, 2H), 1.58 (s, 9H); LCMS (ESI, M+1): m/z=620.1.

Step D. methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of [(2R,8S)-2-fluoro-1,2,3,5,6,7-hexahydropyrrolizin-8-yl]methanol (1.93 g, 1.5 equiv) in THF (100 mL) were added 4 Å molecular sieve (2.0 g) and t-BuONa (2.32 g, 3.0 equiv). The reaction was stirred at 15 °C for 10 minutes. Methyl 4-((7-(2-((tert-butoxycarbonyl)amino)-3-cyano-7-fluorobenzo[b]thiophen-4-yl)-6-chloro-2,8-difluoroquinazolin-4-yl)(methyl)amino)butanoate (5.0 g, 1.0 equiv) was added at 0 °C and the reaction was stirred at 0 °C for 30 minutes. The mixture was added water (100 mL) and extracted with ethyl acetate (2×60 mL). The combined organic layers were dried over anhydrous sodium sulfate and concentrated to afford the title compound (6 g, crude) as a yellow solid.

Step E. 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid: To a solution of methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate (5.0 g, 1.0 equiv) in MeOH (13 mL) was added LiOH (2.0 M, 9 mL, 2.73 equiv). The reaction was stirred at 20 °C for 15 minutes. The mixture was filtered and purified by reversed phase flash [water (0.1% FA)/acetonitrile] to afford the title compound (3.3 g, 66% yield) as yellow solid; LCMS (ESI, M+1): m/z=745.1.

Step F. 3-fluorophenyl 4-((7-(2-((tert-butoxycarbonyl)amino)-3-cyano-7-fluorobenzo[b]thiophen-4-yl)-6-chloro-8-fluoro-2-(((2S,7aR)-2-fluorotetrahydro-1H-pyrrolizin-7a(5H)-yl)methoxy)quinazolin-4-yl)(methyl)amino)butanoate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (100 mg, 1.0 equiv) and 3-fluorophenol (22.6 mg, 1.5 equiv) in DMF (1 mL) were added DMAP (24.6 mg, 1.5 equiv) and EDCI (38.6 mg, 1.5 equiv) at 25 °C. The reaction was stirred at 25 °C for 2 hours. The mixture was diluted with water (2 mL) and extracted with ethyl acetate (3 mL). The organic layer was dried over sodium sulfate, concentrated and purified by reversed phase flash chromatography [C18, 0.1% formic acid condition] to afford the title compound (100 mg, 77% yield) as yellow solid. LCMS (ESI, M+1): m/z=839.2.

Step G. 3-fluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of 3-fluorophenyl 4-((7-(2-((tert-butoxycarbonyl)amino)-3-cyano-7-fluorobenzo[b]thiophen-4-yl)-6-chloro-8-fluoro-2-(((2S,7aR)-2-fluorotetrahydro-1H-pyrrolizin-7a(5H)-yl)methoxy)quinazolin-4-yl)(methyl)amino)butanoate (60.0 mg, 1.0 equiv) in DCM (1 mL) was added TFA (3.07 g, 376 equiv) at 0 °C. The reaction was stirred at 25 °C for 2 hours. The mixture was concentrated under vacuum. The residue was diluted with saturated NaHCO₃ solution (2 mL) and extracted with ethyl acetate (2×2 mL). The combined organic layers were dried over sodium sulfate, concentrated and purified by reversed-phase HPLC (column: Phenomenex luna C18 150×25 mm×10 μm; A: water (FA), B: ACN; B %: 28% to 58% over 9 mins) to afford the title compound (35.2 mg, 66% yield) as white solid. ¹H NMR (400 MHz, DMSO-d6) δ=8.15-8.06 (m, 3H), 7.49-7.41 (m, 1H), 7.26-7.21 (m, 1H), 7.18-7.09 (m, 2H), 7.06 (td, J=2.4, 10.0 Hz, 1H), 6.98 (dd, J=1.6, 8.0 Hz, 1H), 5.48-5.16 (m, 1H), 4.27-4.03 (m, 2H), 3.98-3.79 (m, 2H), 3.47 (s, 3H), 3.29-3.07 (m, 3H), 2.99-2.84 (m, 1H), 2.74 (t, J=7.2 Hz, 2H), 2.31-2.17 (m, 1H), 2.12 (td, J=7.2, 14.4 Hz, 3H), 2.07-1.98 (m, 1H), 1.94-1.76 (m, 3H); LCMS (ESI, M+1): m/z=739.1.

### Example 199

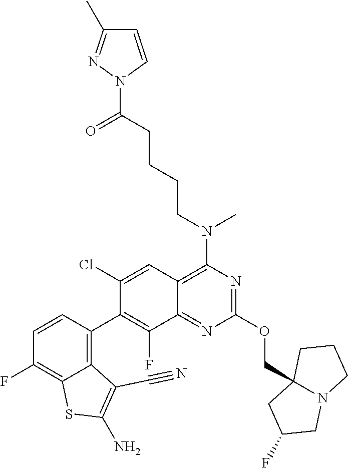 [SMILES: Cc1ccn(C(=O)CCCCN(C)c2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-(methyl(5-(3-methyl-1H-pyrazol-1-yl)-5-oxopentyl)amino)quinazolin-7-yl)-7-fluorobenzo[b]thiophene-3-carbonitrile

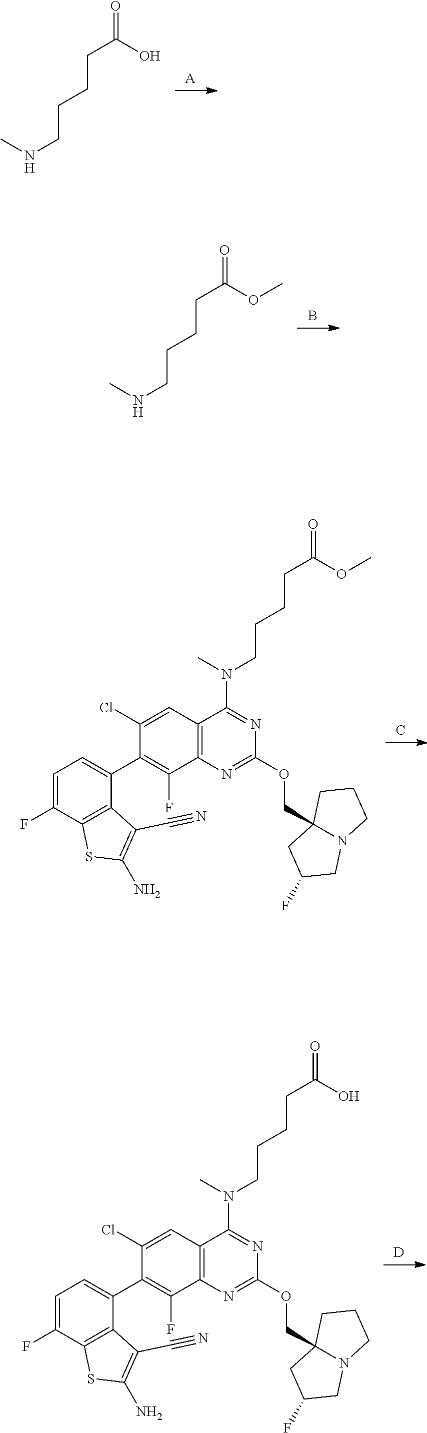

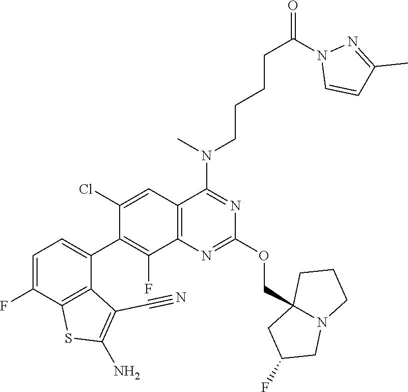 [SMILES: Cc1ccn(C(=O)CCCCN(C)c2nc(OC[C@@]34CCCN3C[C@H](F)C4)nc3c(F)c(-c4ccc(F)c5sc(N)c(C#N)c45)c(Cl)cc23)n1]

Step A. methyl 5-(methylamino)pentanoate: To a solution of 5-(methylamino)pentanoic acid (400 mg, 1.0 equiv) in methanol (2.00 mL) was added thionyl chloride (726 mg, 2.0 equiv) at 25 °C The reaction was stirred at 25 °C for 12 hours. The mixture was directly concentrated to afford the title compound (310 mg, 70% yield) as a yellow solid; ¹H NMR (400 MHz, chloroform-d) δ=9.47 (br s, 2H), 3.67 (s, 3H), 2.97 (br s, 2H), 2.69 (br s, 3H), 2.38 (t, J=7.2 Hz, 2H), 1.97-1.86 (m, 2H), 1.80-1.69 (m, 2H).

Step B. methyl 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)pentanoate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (150 equiv) and methyl 5-(methylamino)pentanoate (79.8 mg, 2.0 equiv) in N,N-dimethylformamide (2.00 mL) were added benzotriazol-1-yloxy(tripyrrolidin-1-yl)phosphonium hexafluorophosphate (214 mg, 1.5 equiv) and diisopropylethylamine (178 mg, 5.0 equiv) at 25 °C. The reaction was stirred at 25 °C for 2 hours. The mixture was quenched by water (15.0 mL) at 25 °C, and then extracted with ethyl acetate (3×20.0 mL). The combined organic layers were washed with brine (3×15.0 mL), dried over anhydrous sodium sulfate, filtered, concentrated under reduced pressure and purified by column chromatography [SiO₂, petroleum ether/ethyl acetate=1/0 to 1/3] to afford the title compound (130 mg, 70% yield) as a yellow solid; LCMS (ESI, M+1): m/z=673.3.

Step C. 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)pentanoic acid: To a solution of methyl 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)pentanoate (120 mg, 1.0 equiv) in methanol (3.00 mL) and water (0.50 mL) was added lithium hydroxide (29.9 mg, 4.0 equiv) at 25 °C. The reaction was stirred at 25 °C for 6 hours. The mixture was filtered. The filtrate was purified by prep-HPLC [Waters Xbridge 150×25 mm×5 μm; A: water (10 mM NH₄HCO₃), B: ACN, B %: 22% to 52% over 9 min] and lyophilized to afford the title compound (45.0 mg, 38% yield) as a yellow solid; LCMS (ESI, M+1): m/z=659.1.

Step D. 2-amino-4-(6-chloro-8-fluoro-2-(((2R,7aS)-2-fluorotetrahydro-1H-pyrrolizin-7a(5H)-yl)methoxy)-4-(methyl(5-(3-methyl-1H-pyrazol-1-yl)-5-oxopentyl)amino)quinazolin-7-yl)-7-fluorobenzo[b]thiophene-3-carbonitrile: To a solution of 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)pentanoic acid (35.0 mg, 1.0 equiv) and 3-methyl-1H-pyrazole (8.72 mg, 2.0 equiv) in N,N-dimethylformamide (0.50 mL) were added EDCI (20.4 mg, 2.0 equiv), HOBt (14.4 mg, 2.0 equiv) and diisopropylethylamine (13.7 mg, 2.0 equiv) at 25 °C. The reaction was stirred at 25 °C for 3 hours. The mixture was filtered. The filtrate was purified by prep-HPLC [Waters Xbridge 150×25 mm×5 μm; A: water (10 mM NH₄HCO₃), B: ACN, B %: 48% to 78% over 15 min] to afford the title compound (5.8 mg, 15% yield) as an off-white solid; ¹H NMR (400 MHz, chloroform-d) δ=8.14 (d, J=2.8 Hz, 1H), 7.85 (s, 1H), 7.16 (ddd, J=1.6, 5.2, 8.4 Hz, 1H), 6.96 (t, J=8.8 Hz, 1H), 6.32-6.14 (m, 3H), 5.36-5.18 (m, 1H), 4.31-4.23 (m, 1H), 4.22-4.16 (m, 1H), 3.84 (qd, J=7.2, 14.4 Hz, 1H), 3.70 (td, J=6.8, 13.6 Hz, 1H), 3.38 (s, 3H), 3.35-3.23 (m, 2H), 3.23-3.14 (m, 3H), 3.04-2.94 (m, 1H), 2.35-2.26 (m, 4H), 2.21 (br d, J=5.6 Hz, 1H), 2.18-2.12 (m, 1H), 1.98-1.81 (m, 7H); LCMS (ESI, M+1): m/z=723.2.

### Example 200

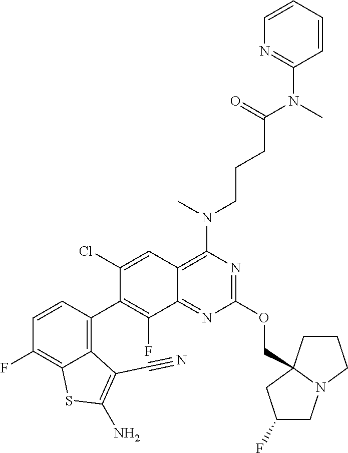 [SMILES: CN(CCCC(=O)N(C)c1ccccn1)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methyl-N-(pyridin-2-yl) butanamide

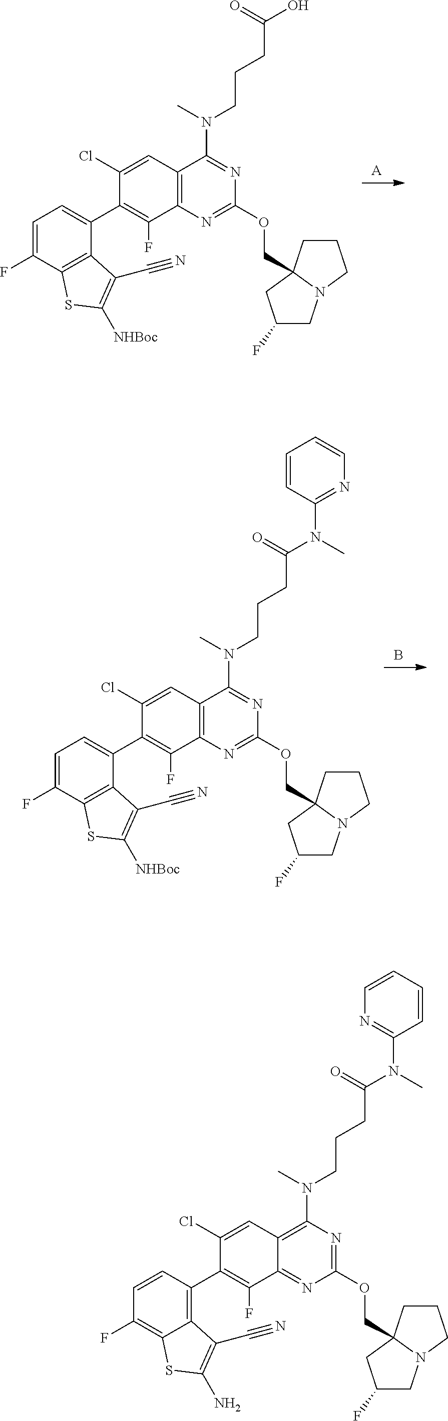

Step tert-butyl(4-(6-chloro-8-fluoro-2-(((2R,7aS)-2-fluorotetrahydro-1H-pyrrolizin-7a(5H)-yl)methoxy)-4-(methyl(4-(methyl(pyridin-2-yl)amino)-4-oxobutyl)amino)quinazolin-7-yl)-3-cyano-7-fluorobenzo[b]thiophen-2-yl)carbamate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (100 mg, 1.0 equiv) and N-methylpyridin-2-amine (90 mg, 6.2 equiv) in THF (1 mL) were added DIEA (128 mg, 7.4 equiv) and CMPI (169 mg, 4.9 equiv). The reaction was stirred at 25 °C for 12 hours. The mixture was diluted with water (5 mL) and extracted with ethyl acetate (3 mL). The organic layer was dried over sodium sulfate and concentrated to afford the title compound (100 mg, crude) as yellow oil.

Step B. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methyl-N-(pyridin-2-yl) butanamide: To a solution of tert-butyl(4-(6-chloro-8-fluoro-2-(((2R,7aS)-2-fluorotetrahydro-1H-pyrrolizin-7a(5H)-yl)methoxy)-4-(methyl(4-(methyl(pyridin-2-yl)amino)-4-oxobutyl)amino)quinazolin-7-yl)-3-cyano-7-fluorobenzo[b]thiophen-2-yl)carbamate (100 mg, 1.0 equiv) in DCM (1 mL) was added TFA (1.54 g, 112 equiv) at 25 °C. The reaction was stirred at 25 °C for 0.5 hours. The mixture was concentrated. The residue was dissolved in ethyl acetate (5 mL) and basified with saturated NaHCO₃ solution (10 mL). The mixture was extracted with ethyl acetate (2×5 mL). The combined organic layers were dried over anhydrous sodium sulfate, concentrated and purified by prep-HPLC [column: Phenomenex luna C18 250×50 mm×15 μm; A: water (FA), B: ACN, B %: 20%-50% B over 9 min] to afford the title compound (12.2 mg, 13.7% yield) as yellow solid; ¹H NMR (400 MHz, DMSO-d6) δ=8.43 (dd, J=1.2, 4.8 Hz, 1H), 8.16-7.99 (m, 3H), 7.86-7.82 (m, 1H), 7.42 (br d, J=8.4 Hz, 1H), 7.29-7.23 (m, 2H), 7.19-7.11 (m, 1H), 5.61-5.26 (m, 1H), 4.49-4.09 (m, 2H), 3.87-3.44 (m, 5H), 3.39 (s, 3H), 3.24 (s, 3H), 3.15-2.90 (m, 1H), 2.41 (br t, J=6.3 Hz, 2H), 2.34-1.64 (m, 8H); LCMS (ESI, M+1): m/z=735.3.

### Example 201

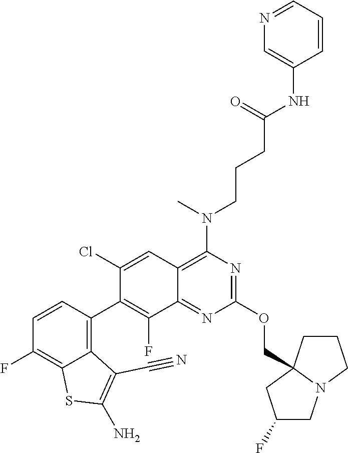 [SMILES: CN(CCCC(=O)Nc1cccnc1)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(pyridin-3-yl)butanamide

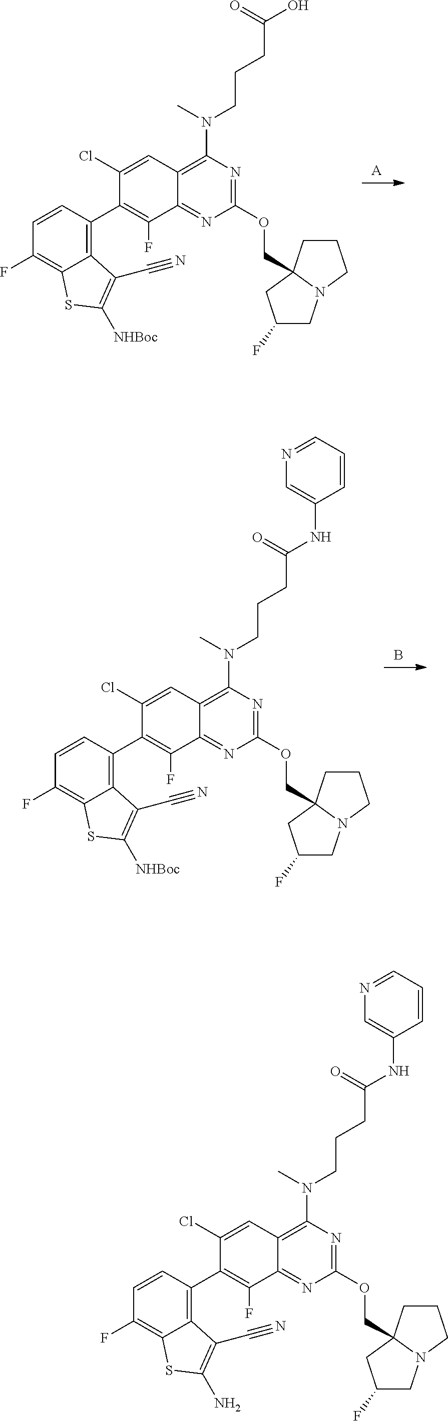

Step A. tert-butyl(4-(6-chloro-8-fluoro-2-(((2R,7aS)-2-fluorotetrahydro-1H-pyrrolizin-7a(5H)-yl)methoxy)-4-(methyl(4-oxo-4-(pyridin-3-ylamino)butyl)amino)quinazolin-7-yl)-3-cyano-7-fluorobenzo[b]thiophen-2-yl)carbamate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (50.0 mg, 1.0 equiv) in 2-methyltetrahydrofuran (0.5 mL) were added DIEA (43.4 mg, 5.0 equiv), pyridin-3-amine (9.47 mg, 1.5 equiv) and 2-chlor-1-methylpyridinium (25.7 mg, 1.5 equiv). The reaction was stirred at 40 °C for 2 hours. The mixture was diluted with water (10 mL) and extracted with ethyl acetate (3×10 mL). The combined organic layers were washed with brine (10 mL), dried over anhydrous sodium sulfate and concentrated to afford the title compound (70.0 mg, crude) as yellow solid; LCMS (ESI, M+1): m/z=821.3.

Step B. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(pyridin-3-yl) butanamide: To a solution of tert-butyl(4-(6-chloro-8-fluoro-2-(((2R,7aS)-2-fluorotetrahydro-1H-pyrrolizin-7a(5H)-yl)methoxy)-4-(methyl(4-oxo-4-(pyridin-3-ylamino)butyl)amino)quinazolin-7-yl)-3-cyano-7-fluorobenzo[b]thiophen-2-yl)carbamate (68.0 mg, 1.0 equiv) in MeCN (1 mL) was added HCl·dioxane (2 M, 1.0 mL). The reaction was stirred at 25 °C for 2 hours. The mixture was concentrated. The residue was dissolved in MeOH (1 mL) and adjusted to pH=7 with NaHCO₃solid. The mixture was filtered, concentrated and purified with prep-HPLC [column: Phenomenex luna C18 150×25 mm×10 μm; mobile phase: [water (FA)-ACN]; gradient: 15%-45% B over 15 min] to afford the title compound (4.07 mg, 6.8% yield) as white solid; ¹H NMR (400 MHz, METHANOL-d₄) δ=8.67 (s, 1H), 8.23 (d, J=4.8 Hz, 1H), 8.08 (s, 1H), 8.01 (br d, J=8.4 Hz, 1H), 7.36 (dd, J=4.8, 8.0 Hz, 1H), 7.19 (dd, J=5.2, 8.4 Hz, 1H), 7.05 (t, J=9.2 Hz, 1H), 5.49-5.35 (m, 1H), 4.50-4.38 (m, 2H), 4.14-4.07 (m, 1H), 3.95-3.86 (m, 1H), 3.73-3.63 (m, 2H), 3.56 (s, 3H), 3.23 (dt, J=5.9, 9.8 Hz, 2H), 2.57-2.54 (m, 2H), 2.49-1.99 (m, 8H); LCMS (ESI, M+1): m/z=721.3.

### Example 202

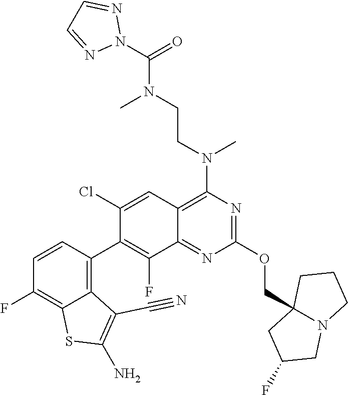 [SMILES: CN(CCN(C)c1nc(OC[C@@]23CCCN2C[C@H](F)C3)nc2c(F)c(-c3ccc(F)c4sc(N)c(C#N)c34)c(Cl)cc12)C(=O)n1nccn1]

### N-(2-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-2H-1,2,3-triazole-2-carboxamide

### Example 203

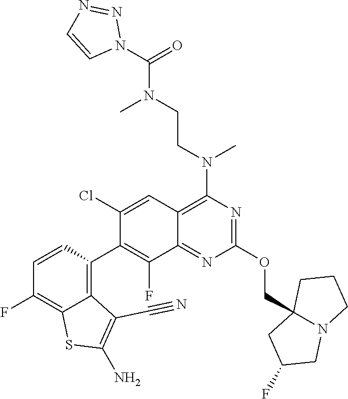

### N-(2-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-1H-1,2,3-triazole-1-carboxamide

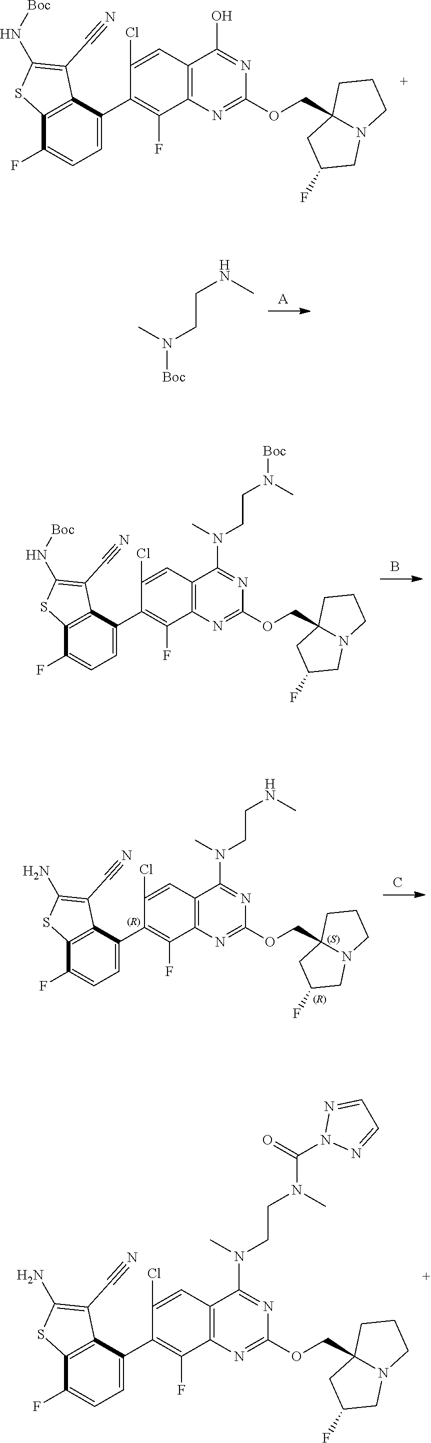

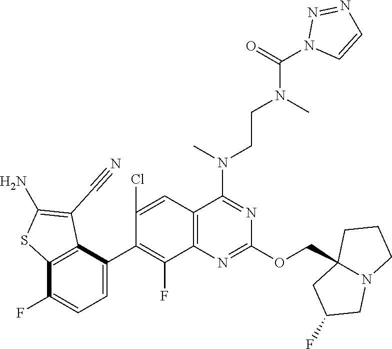

Step A. tert-butyl(2-(((S)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)(methyl)carbamate: To a solution of tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1.0 equiv) and tert-butyl N-methyl-N-[2-(methylamino)ethyl]carbamate (175 mg, 1.2 equiv) in dimethylformamide (5.00 mL) were added diisopropylethylamine (300 mg, 3.0 equiv) and benzotriazol-1-yloxy(tripyrrolidin-1-yl)phosphonium hexafluorophosphate (564 mg, 1.4 equiv) at 25 °C. The reaction was stirred at 25 °C for 2 hours. The mixture was quenched by addition water (20.0 mL) at 25 °C. The yellow solid was formed, filtered and the filter cake was washed with water (5.00 mL). The cake was dried in vacuum to afford the title compound (550 mg, 87% yield) as a yellow solid; LCMS (ESI, M+1): m/z=816.4.

Step B. 2-amino-4-((S)-6-chloro-8-fluoro-2-(((2R,7aS)-2-fluorotetrahydro-1H-pyrrolizin-7a(5H)-yl)methoxy)-4-(methyl(2-(methylamino)ethyl)amino)quinazolin-7-yl)-7-fluorobenzo[b]thiophene-3-carbonitrile: To a solution of tert-butyl(2-(((S)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)(methyl)carbamate (550 mg, 1.0 equiv) in dichloromethane (3 mL) was added trifluoroacetic acid (1.5 mL) at 25 °C. The reaction was stirred at 25 °C for 2 hours. The mixture was concentrated. The residue was diluted with methyl alcohol (20.0 mL) and adjusted to pH=8 with sodium bicarbonate solid, and then filtered. The filtrate was concentrated under reduced pressure and purified by reversed-phase chromatography [C18, 0.1% formic acid condition] to afford the title compound (290 mg, 70% yield) as a yellow solid; ¹H NMR (400 MHz, chloroform-d) δ=7.97 (s, 1H), 7.15 (br dd, J=4.8, 8.0 Hz, 1H), 6.95 (br t, J=8.8 Hz, 1H), 6.61 (br s, 2H), 5.40-5.13 (m, 1H), 4.24 (br d, J=10.4 Hz, 1H), 4.16-4.10 (m, 1H), 3.41 (s, 3H), 3.33-3.08 (m, 4H), 3.06-2.88 (m, 3H), 2.49 (s, 3H), 2.28-2.09 (m, 3H), 2.02-1.77 (m, 4H); LCMS (ESI, M+1): m/z=616.3.

Step C. N-(2-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-2H-1,2,3-triazole-2-carboxamide and N-(2-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-1H-1,2,3-triazole-1-carboxamide: To a solution of 1H-triazole (33.6 mg, 1.0 equiv) in dichloromethane (3 mL) were added diisopropylethylamine (378 mg, 6.0 equiv) and bis(trichloromethyl) carbonate (150 mg, 1.1 equiv) at 0 °C. The reaction was stirred at 0 °C for 1 hour. To the above mixture was added a solution of 2-amino-4-((S)-6-chloro-8-fluoro-2-(((2R,7aS)-2-fluorotetrahydro-1H-pyrrolizin-7a(5H)-yl)methoxy)-4-(methyl(2-(methylamino)ethyl)amino)quinazolin-7-yl)-7-fluorobenzo[b]thiophene-3-carbonitrile (270 mg, 0.90 equiv) in dichloromethane (3 mL) at 0 °C. The reaction was at 0 °C for 12 hours. The mixture was concentrated and purified by prep-HPLC [Waters Xbridge 150×25 mm×5 μm; A: water (10 mM NH₄HCO₃), B: ACN, B %: 45%-75% B over 9 min] and SFC [column: DAICEL CHIRALPAK AD (250 mm×30 mm, 10 μm); A: CO₂-ACN, B: i-PrOH (0.1% NH₃·H₂O); B %: 50%, isocratic elution mode] to afford two peaks.

N-(2-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-2H-1,2,3-triazole-2-carboxamide: Peak 1 was purified with prep-HPLC [Waters Xbridge150×25 mm×5 μm; A: water (10 mM NH₄HCO₃), B: ACN, B %: 44% to 74% over 9 min] to afford the title compound (6.37 mg, 1.8% yield) as an off-white solid; ¹H NMR (400 MHz, CDCl₃) δ=8.08 (br s, 1H), 8.00-7.83 (m, 1H), 7.69 (br s, 1H), 7.19 (dd, J=5.2, 8.4 Hz, 1H), 7.00 (t, J=8.8 Hz, 1H), 5.88 (br s, 2H), 5.45-5.15 (m, 1H), 4.37-3.92 (m, 6H), 3.63-3.16 (m, 9H), 2.99 (br s, 1H), 2.45-1.99 (m, 4H), 1.89-1.58 (m, 2H); LCMS (ESI, M+1): m/z=711.2; SFC: 99% de, Column: Chiralpak AD-3 50×4.6 mm I.D., 3 μm; Mobile phase: Phase A for CO₂, and Phase B for IPA+ACN (0.05% DEA); Gradient elution: IPA+ACN (0.05% DEA) in CO₂ from 20% to 60%; Flow rate: 3 mL/min; Detector: PDA; Column Temp: 35C; Back Pressure: 100 Bar; T_{R}: 1.857 min.

N-(2-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-1H-1,2,3-triazole-1-carboxamide: Peak 2 was purified with prep-HPLC [Waters Xbridge150×25 mm×5 μm; A: water (10 mM NH₄HCO₃), B: ACN, B %: 44%-74% B over 9 min] to afford the title compound (10.6 mg, 3.0% yield) as an off-white solid; ¹H NMR (400 MHz, CDCl₃) δ=7.94 (br s, 1H), 7.85-7.63 (m, 2H), 7.18 (dd, J=5.2, 8.4 Hz, 1H), 6.99 (t, J=8.8 Hz, 1H), 6.16 (br s, 2H), 5.43-5.12 (m, 1H), 4.29-3.76 (m, 6H), 3.71-3.44 (m, 3H), 3.41-3.16 (m, 6H), 3.07-2.91 (m, 1H), 2.03 (s, 4H), 1.93-1.71 (m, 2H); LCMS (ESI, M+1): m/z=711.1; SFC: 98% de, Column: Chiralpak AD-3 50×4.6 mm I.D., 3 μm; Mobile phase: Phase A for CO₂, and Phase B for IPA+ACN (0.05% DEA); Gradient elution: IPA+ACN (0.05% DEA) in CO₂ from 20% to 60%; Flow rate: 3 mL/min; Detector: PDA; Column Temp: 35C; Back Pressure: 100 Bar; T_{R}: 2.007 min.

### Example 204

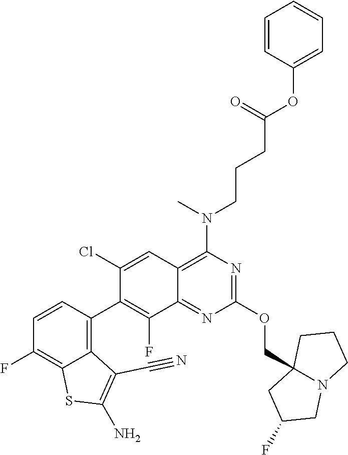 [SMILES: CN(CCCC(=O)Oc1ccccc1)c1nc(OC[C@@]23CCCN2C[C@H](F)C3)nc2c(F)c(-c3ccc(F)c4sc(N)c(C#N)c34)c(Cl)cc12]

### phenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate

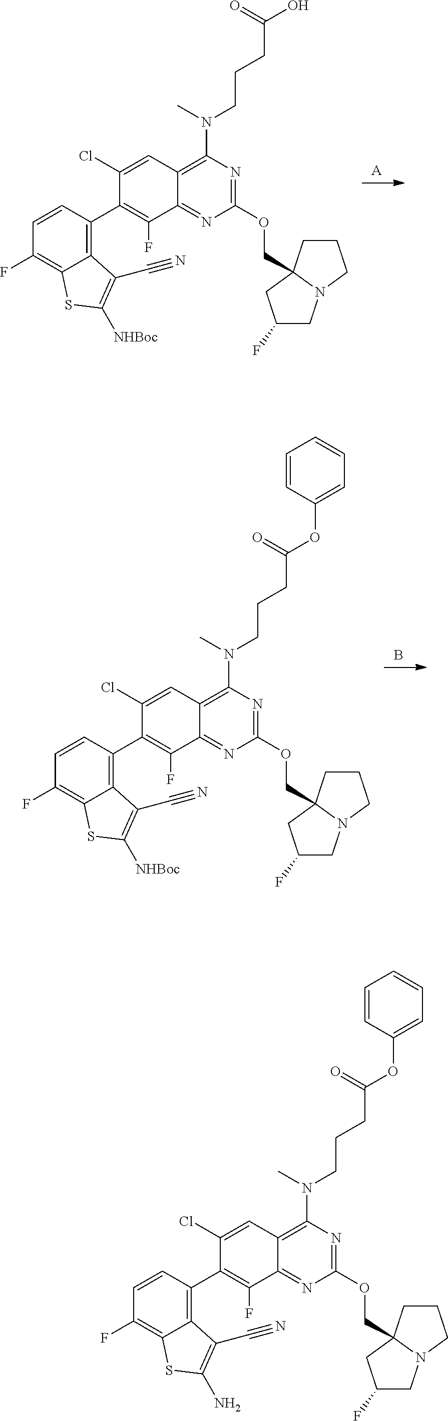

Step A. phenyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (100 mg, 1.0 equiv) and phenol (18.9 mg, 1.5 equiv) in DMF (2 mL) were added EDCI (38.6 mg, 1.5 equiv) and DMAP (24.6 mg, 1.5 equiv). The reaction was stirred at 60 °C for 5 hours. The mixture was purified by reversed phase flash [water (0.1% FA)/acetonitrile] to afford the title compound (50 mg, 45% yield) as yellow oil; LCMS (ESI, M+1): m/z=821.3.

Step B. phenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of phenyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate (45 mg, 1.0 equiv) in DCM (0.5 mL) was added TFA (767 mg, 123 equiv). The reaction was stirred at 20 °C for 0.5 hours. The mixture was concentrated in vacuum, basified with saturated aqueous NaHCO₃ (2 mL) and extracted with ethyl acetate (2×2 mL). The combined organic layers were washed with NaHCO₃ (3×3 mL), dried over anhydrous sodium sulfate, concentrated in vacuum and purified by prep-HPLC [column: Waters Xbridge 150×25 mm×5 μm; A: water (ammonia hydroxide v/v), B: ACN, B %: 50%-80% over 10 min] to afford the title compound (10.3 mg, 25% yield) as white solid; ¹H NMR (400 MHz, METHANOL-d₄) δ=8.13 (d, J=1.6 Hz, 1H), 7.44-7.35 (m, 2H), 7.30-7.18 (m, 2H), 7.12-7.02 (m, 3H), 5.39-5.19 (m, 1H), 4.40-4.25 (m, 2H), 4.07-3.95 (m, 2H), 3.60 (s, 3H), 3.52-3.39 (m, 1H), 3.32-3.13 (m, 2H), 3.11-3.01 (m, 1H), 2.78 (t, J=7.2 Hz, 2H), 2.45-2.18 (m, 4H), 2.16-2.08 (m, 1H), 2.03-1.93 (m, 2H), 1.92-1.83 (m, 1H); LCMS (ESI, M+1): m/z=721.2.

### Example 205

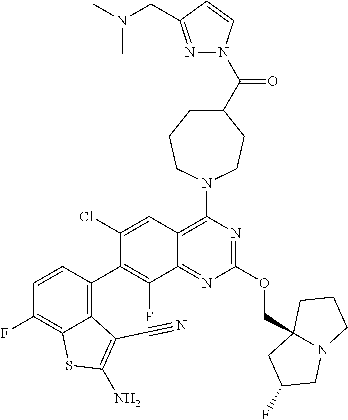 [SMILES: CN(C)Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-4-(4-(3-((dimethylamino)methyl)-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

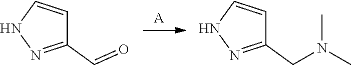

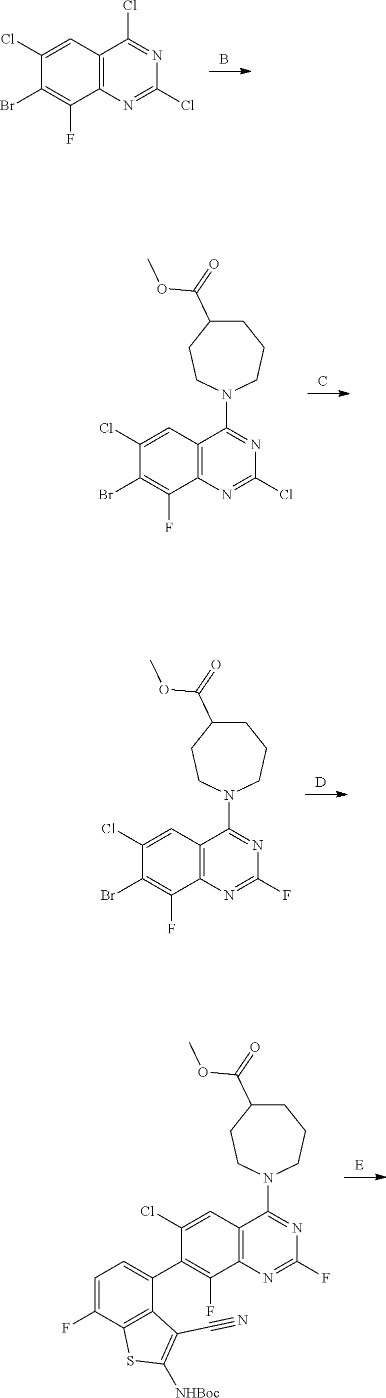

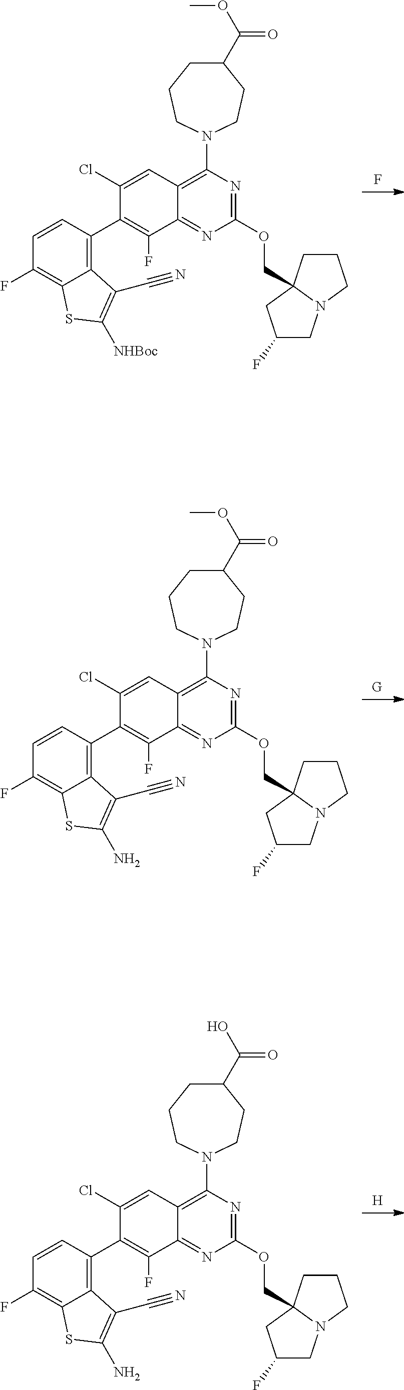

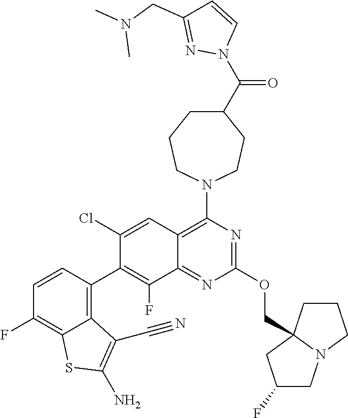 [SMILES: CN(C)Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

Step A. N,N-dimethyl-1-(1H-pyrazol-3-yl) methanamine: To a solution of 1H-pyrazole-3-carbaldehyde (1.00 g, 1.0 equiv) in MeOH (10 mL) was added N-methylmethanamine (4.87 g, 40% purity, 4.1 equiv) at 0 °C. The reaction was stirred at 0 °C for 1 hour. NaBH₃CN (1.64 g, 2.5 equiv) was added into above mixture at 0 °C. The reaction was stirred at 20 °C for 14 hours. The mixture was quenched with saturated K₂CO₃ aqueous (1 mL) and purified with reversed phase flash chromatography [water (0.1% NH₃·H₂O)/acetonitrile=4/1] and prep-HPLC [column: Waters xbridge 150×25 mm×10 μm; mobile phase: water (ammonia hydroxide v/v)-acetonitrile; gradient: 1%-20% B over 10 minutes] to afford the title compound (275 mg, 21% yield) as light yellow oil; LCMS (ESI, M+1): m/z=126.2.

Step B. Methyl 1-(7-bromo-2,6-dichloro-8-fluoroquinazolin-4-yl)azepane-4-carboxylate: To a solution of 7-bromo-2,4,6-trichloro-8-fluoroquinazoline (10.0 g, 1.0 equiv) and DIEA (19.6 g, 5.0 equiv) in DCM (100 mL) was added methyl azepane-4-carboxylate (5.23 g, 1.1 equiv) at −40 °C. The reaction was stirred at −40 °C for 0.5 hours. The mixture was diluted with water (40 mL) and extracted with DCM (3×30 mL). The combined organic layers were dried over anhydrous sodium sulfate, concentrated and triturated with acetonitrile at 20 °C for 0.5 hours to afford the title compound (9.80 g, 72% yield) as yellow solid; LCMS (ESI, M+1, M+3): m/z=450.1, 452.1.

Step C. methyl 1-(7-bromo-6-chloro-2,8-difluoroquinazolin-4-yl)azepane-4-carboxylate: To a mixture of methyl 1-(7-bromo-2,6-dichloro-8-fluoroquinazolin-4-yl)azepane-4-carboxylate (8.80 g, 1.0 equiv) and 18-crown-6 ether (5.16 g, 1.0 equiv) in DMSO (88 mL) was added KF (11.3 g, 10.0 equiv). The reaction was stirred at 130 °C for 1 hour. The mixture was diluted with water (80 mL) and extracted with ethyl acetate (3×40 mL). The combined organic layers were washed with brine (3×120 mL), dried over anhydrous sodium sulfate and concentrated to afford the title compound (160 mg, crude) as yellow solid; LCMS (ESI, M+1, M+3): m/z=434.0, 436.0.

Step D. methyl 1-(7-(2-((tert-butoxycarbonyl)amino)-3-cyano-7-fluorobenzo[b]thiophen-4-yl)-6-chloro-2,8-difluoroquinazolin-4-yl)azepane-4-carboxylate: To a mixture of methyl 1-(7-bromo-6-chloro-2,8-difluoroquinazolin-4-yl)azepane-4-carboxylate (1.5 g, 1.0 equiv), tert-butyl(3-cyano-4-(5,5-dimethyl-1,3,2-dioxaborinan-2-yl)-7-fluorobenzo[b]thiophen-2-yl)carbamate (1.67 g, 1.2 equiv) and Cs₂CO₃ (3.37 g, 3.0 equiv) in THF (15 mL) was added Pd(DPEphos)Cl₂ (494 mg, 0.2 equiv). The reaction was degassed and purged with nitrogen 3 times. The reaction was stirred at 60 °C for 3 hours. The mixture was filtered and concentrated and purified by column chromatography (SiO₂, Petroleum ether/Ethyl acetate=1/0 to 3/1) and reversed phase flash [C18, 0.1% formic acid condition] to afford the title compound (4.80 g, 52% yield) as yellow liquid; LCMS (ESI, M+1): m/z=646.3.

Step E. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a mixture of ((2R,7aS)-2-fluorotetrahydro-1H-pyrrolizin-7a(5H)-yl)methanol (1.18 g, 1.2 equiv), t-BuONa (773 mg, 1.3 equiv) and 4 Å molecular sieve (100 mg) in THF (40 mL) was added methyl 1-(7-(2-((tert-butoxycarbonyl)amino)-3-cyano-7-fluorobenzo[b]thiophen-4-yl)-6-chloro-2,8-difluoroquinazolin-4-yl)azepane-4-carboxylate (4.00 g, 1.0 equiv) at 0 °C. The reaction was stirred at 0 °C for 2 hours. The mixture was filtered. The filtrate was diluted with water (40 mL) and extracted with ethyl acetate (3×30 mL). The combined organic layers were dried over anhydrous sodium sulfate, concentrated and purified by reversed phase flash [C18, 0.1% formic acid condition] to afford the title compound (2.7 g, 55% yield) as white solid; LCMS (ESI, M+1): m/z=785.3.

Step F. methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (2.2 g, 1.0 equiv) in DCM (10 mL) was added TFA (30.7 g, 96 equiv). The reaction was stirred at 20 °C for 0.5 hours. The mixture was concentrated. The residue was diluted with water (10 mL) and adjusted pH to 8 with saturated NaHCO₃ aqueous. The mixture was extracted with DCM (3×15 mL). The combined organic layers were dried over anhydrous sodium sulfate and concentrated to afford the title compound (1.90 g, 99% yield) as yellow solid; LCMS (ESI, M+1): m/z=685.2.

Step G. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (1.80 g, 1.0 equiv) in THF (6 mL), H₂O (6 mL) and MeOH (4 mL) was added LiOH·H₂O (331 mg, 3.0 equiv). The reaction was stirred at 20 °C for 2 hours. The mixture was filtered and purified by reversed phase flash [C18, 0.1% formic acid condition] to afford the title compound (1.30 g, 71% yield) as yellow solid; LCMS (ESI, M+1): m/z=671.3.

Step H. 2-amino-4-(6-chloro-4-(4-(3-((dimethylamino)methyl)-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (60.0 mg, 1.0 equiv) and N,N-dimethyl-1-(1H-pyrazol-3-yl) methanamine (28.0 mg, 2.5 equiv) in DMAc (1 mL) were added DIEA (57.8 mg, 5.0 equiv) and HATU (34.0 mg, 1.0 equiv). The reaction was stirred at 20 °C for 14 hours. The mixture was purified with reversed phase flash [water (0.1% formic acid)/acetonitrile=2/1]. The desired fraction was collected and basified by NaHCO₃ (0.3 g). The mixture was concentrated to remove acetonitrile and then extracted with ethyl acetate (3×5 mL). The combined organic layers were washed brine (10 mL), dried over anhydrous sodium sulfate and concentrated to afford the title compound (15.2 mg, 21% yield) as off-white solid; ¹H NMR (400 MHz, DMSO-d₆) δ=8.32 (d, J=2.8 Hz, 1H), 8.11 (br s, 2H), 8.03-7.95 (m, 1H), 7.28-7.19 (m, 1H), 7.19-7.10 (m, 1H), 6.53 (t, J=2.4 Hz, 1H), 5.37-5.16 (m, 1H), 4.27-3.89 (m, 5H), 3.89-3.69 (m, 2H), 3.48-3.37 (m, 2H), 3.14-3.03 (m, 2H), 3.00 (br s, 1H), 2.86-2.75 (m, 1H), 2.35-2.24 (m, 1H), 2.19-1.96 (m, 13H), 1.85-1.62 (m, 4H); LCMS (ESI, M+1): m/z=778.3.

### Example 206

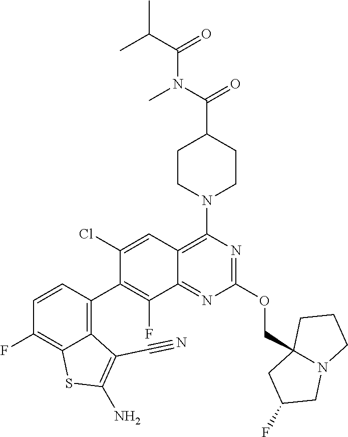 [SMILES: CC(C)C(=O)N(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1]

### 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-isobutyryl-N-methylpiperidine-4-carboxamide

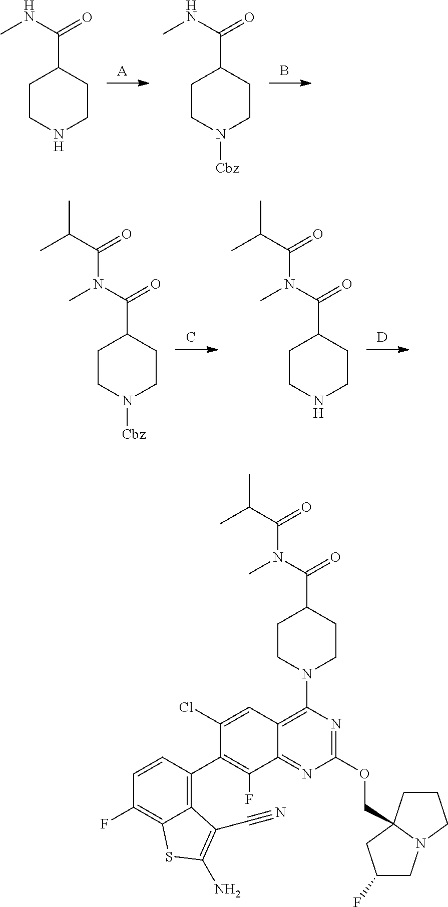

Step A. benzyl 4-(methylcarbamoyl)piperidine-1-carboxylate: To a solution of N-methylpiperidine-4-carboxamide (HCl, 3.00 g, 1.0 equiv), TEA (10.2 g, 6.0 equiv) in DCM (30 mL) was added CbzCl (2.86 g, 1.0 equiv) dropwise at −40 °C. The reaction was stirred at −40 °C for 2 hours. The mixture was diluted with H₂O (50 mL) and extracted with Ethyl acetate (3×20 mL). The combined organic layers were washed with HCl aqueous (1 M, 50 mL), dried over anhydrous sodium sulfate and concentrated to afford the title compound (2.20 g, 47% yield) as white solid; ¹H NMR (400 MHz, DMSO-d₆) δ=7.77 (br d, J=4.4 Hz, 1H), 7.41-7.27 (m, 5H), 5.07 (s, 2H), 4.02-3.96 (m, 2H), 2.91-2.71 (m, 2H), 2.55 (d, J=4.4 Hz, 3H), 2.34-2.23 (m, 1H), 1.66 (br d, J=11.2 Hz, 2H), 1.41 (dq, J=4.4, 12.4 Hz, 2H).

Step B. benzyl 4-(isobutyryl(methyl)carbamoyl)piperidine-1-carboxylate: To a solution of benzyl 4-(methylcarbamoyl)piperidine-1-carboxylate (200 mg, 1.0 equiv), CMPI (925 mg, 5.0 equiv) in 2-methyltetrahydrofuran (2 mL) were added TEA (1.10 g, 15 equiv) and isobutyric acid (319 mg, 5.0 equiv). The reaction was stirred at 80 °C for 12 hours. The mixture was diluted with H₂O (5 mL) and extracted with Ethyl acetate (3×2 mL). The combined organic layers were washed with brine (5 mL), dried over anhydrous sodium sulfate, concentrated and purified by reverse-phase flash chromatography [C18, 0.1% formic acid condition] to afford the title compound (180 mg, 47% yield) as yellow solid; ¹H NMR (400 MHz, CHLOROFORM-d) δ=7.40-7.29 (m, 5H), 5.14 (s, 2H), 4.22 (br s, 2H), 3.31 (tt, J=3.6, 11.2 Hz, 1H), 3.24 (s, 3H), 3.10 (td, J=6.8, 13.6 Hz, 1H), 2.88 (br s, 2H), 1.84 (br s, 2H), 1.68 (dq, J=4.4, 12.0 Hz, 3H), 1.20 (d, J=6.8 Hz, 6H); LCMS (ESI, M+1): m/z=347.1.

Step C. N-isobutyryl-N-methylpiperidine-4-carboxamide: To a solution of benzyl 4-(isobutyryl(methyl) carbamoyl)piperidine-1-carboxylate (1800 mg, 1.0 equiv) in Ethyl acetate (6 mL) was added Pd/C (52 mg, 10% purity) under N₂ atmosphere. The suspension was degassed and purged with H₂ for 3 times. The reaction was stirred at 25 °C for 1 hour under H₂ (15 psi) atmosphere. The mixture was filtered through a pad of Celite and concentrated to afford the title compound (110 mg, crude) as white solid; ¹H NMR (400 MHz, CHLOROFORM-d) δ=3.25 (s, 1H), 3.24 (s, 2H), 3.21-3.14 (m, 2H), 2.82 (t, J=2.4 Hz, 1H), 2.75-2.67 (m, 2H), 1.85 (br d, J=13.2 Hz, 2H), 1.78-1.60 (m, 3H), 1.19 (d, J=6.8 Hz, 6H).

Step D. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-isobutyryl-N-methylpiperidine-4-carboxamide: To a solution of N-isobutyryl-N-methylpiperidine-4-carboxamide (100 mg, 1.0 equiv), 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (257 mg, 1.0 equiv) in DMSO (2 mL) were added PyBOP (245 mg, 1.0 equiv) and DIEA (183 mg, 3.0 equiv). The reaction was stirred at 25 °C for 2 hours. The mixture was filtered and purified by prep-HPLC [Phenomenex Luna C18 150×25 mm×10 μm; A: water (FA), B: ACN, B %: 22%-52% over 65 min], followed by prep-HPLC [column: Waters Xbridge C18 150×25 mm×5 μm; mobile phase: [water (NH₄HCO₃)-ACN]; gradient: 60%-90% B over 9 min] to afford the title compound (13.3 mg, 3.7%) as yellow solid. ¹H NMR (400 MHz, CHLOROFORM-d) δ=7.73 (s, 1H), 7.21 (dd, J=5.2, 8.4 Hz, 1H), 7.03 (t, J=8.8 Hz, 1H), 5.66-5.43 (m, 2H), 5.43-5.21 (m, 1H), 4.56-3.98 (m, 4H), 3.61-3.50 (m, 1H), 3.35 (br s, 2H), 3.29 (s, 3H), 3.27-3.20 (m, 2H), 3.19 (br d, J=1.6 Hz, 1H), 3.03 (br s, 1H), 2.59-2.15 (m, 3H), 2.15-1.79 (m, 7H), 1.73-1.64 (m, 1H), 1.29-1.17 (m, 6H); LCMS (ESI, M+1): m/z=740.5.

### Example 207

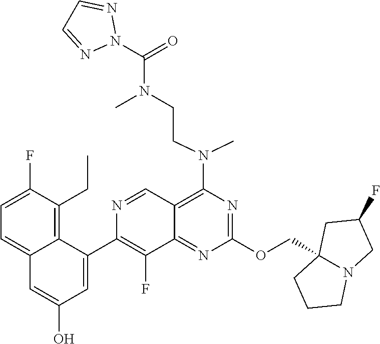 [SMILES: CCc1c(F)ccc2cc(O)cc(-c3ncc4c(N(C)CCN(C)C(=O)n5nccn5)nc(OC[C@@]56CCCN5C[C@H](F)C6)nc4c3F)c12]

### N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-2H-1,2,3-triazole-2-carboxamide

### Example 208

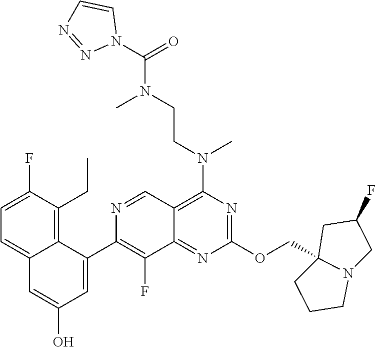 [SMILES: CCc1c(F)ccc2cc(O)cc(-c3ncc4c(N(C)CCN(C)C(=O)n5ccnn5)nc(OC[C@@]56CCCN5C[C@H](F)C6)nc4c3F)c12]

### N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-1H-1,2,3-triazole-1-carboxamide

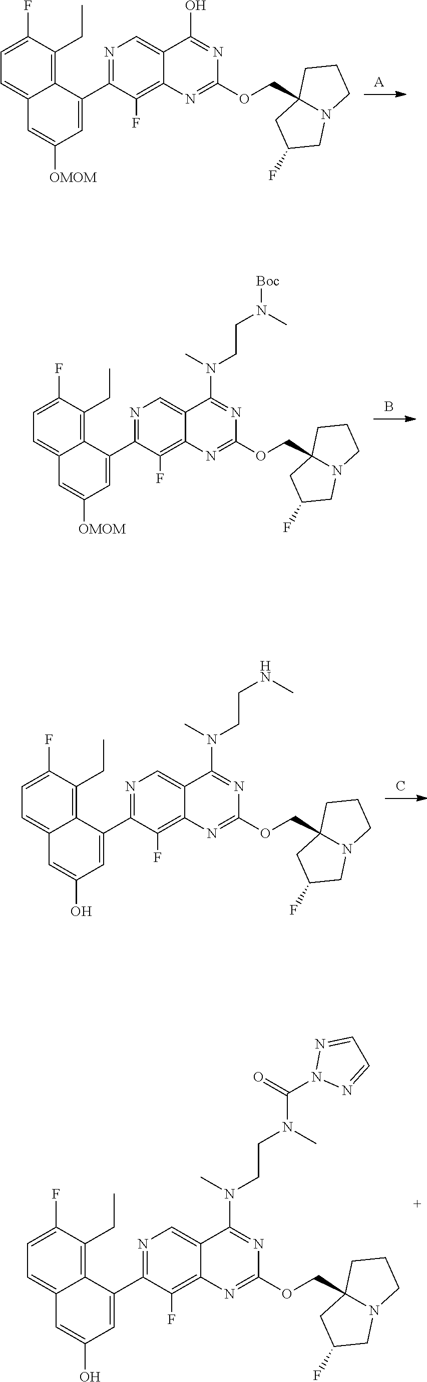

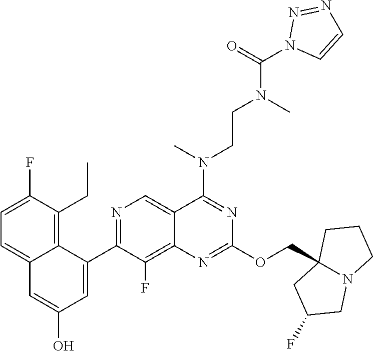 [SMILES: CCc1c(F)ccc2cc(O)cc(-c3ncc4c(N(C)CCN(C)C(=O)n5ccnn5)nc(OC[C@@]56CCCN5C[C@H](F)C6)nc4c3F)c12]

Step A. tert-butyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)(methyl)carbamate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 equiv) and tert-butyl N-methyl-N-[2-(methylamino)ethyl]carbamate (509 mg, 3.0 equiv) in dimethyl formamide (5.00 mL) were added benzotriazol-1-yloxy(tripyrrolidin-1-yl)phosphonium hexafluorophosphate (938 mg, 2.0 equiv) and diisopropylethylamine (583 mg, 5.0 equiv) at 25 °C. The reaction was stirred at 25 °C for 12 hours. The mixture was quenched by addition ice water (15.0 mL) at 0 °C, and then extracted with dichloromethane (2×15.0 mL). The combined organic layers were washed with brine (2×10.0 mL), dried over anhydrous sodium sulfate, filtered, concentrated under reduced pressure and purified by flash silica gel chromatography [SiO₂, DCM/MeOH=1/0 to 10/1] to afford the title compound (620 mg, 95% yield) as a yellow solid; LCMS (ESI, M+1): m/z=725.6.

Step B. 5-ethyl-6-fluoro-4-(8-fluoro-2-(((2R,7aS)-2-fluorotetrahydro-1H-pyrrolizin-7a(5H)-yl)methoxy)-4-(methyl(2-(methylamino)ethyl)amino)pyrido [4,3-d]pyrimidin-7-yl)naphthalen-2-ol: To a solution of tert-butyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)(methyl)carbamate (620 mg, 1.0 equiv) in dichloromethane (3 mL) was added trifluoroacetic acid (1.40 g, 18.0 equiv) at 25 °C. The reaction was stirred at 25 °C for 2 hours. The mixture was concentrated under reduced pressure and purified by prep-HPLC [Phenomenex luna C18 150×40 mm×15 μm; A: water (0.1% FA), B: ACN, B %: 5% to 35% over 15 min] to afford the title compound (350 mg, 88% yield) as a white solid; LCMS (ESI, M+1): m/z=581.4

Step C. N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-2H-1,2,3-triazole-2-carboxamide and N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-1H-1,2,3-triazole-1-carboxamide: To a solution of 1H-triazole (143 mg, 4.0 equiv) and triethylamine (261 mg, 5.0 equiv) in dichloromethane (10.0 mL) was added a solution of bis(trichloromethyl) carbonate (307 mg, 2.0 equiv) in dichloromethane (5.00 mL) at 0 °C. The reaction was stirred at 0 °C for 1 hour. A solution of 5-ethyl-6-fluoro-4-(8-fluoro-2-(((2R,7aS)-2-fluorotetrahydro-1H-pyrrolizin-7a(5H)-yl)methoxy)-4-(methyl(2-(methylamino)ethyl)amino)pyrido [4,3-d]pyrimidin-7-yl)naphthalen-2-ol (300 mg, 1.0 equiv) in dichloromethane (5 mL) was added to above mixture. The reaction was stirred at 0 °C for 12 hours. The mixture was quenched by addition ice water (20.0 mL) at 0 °C, and extracted with ethyl acetate (3×40.0 mL). The combined organic layers were washed with brine (3×20.0 mL), dried over anhydrous sodium sulfate, filtered, concentrated under reduced pressure and purified by prep-HPLC [YMC-Actus Triart C18 150×30 mm×7 μm; A: water (0.1% FA), B: ACN, B %: 20%-50% over 10 min] and SFC [condition: column: DAICEL CHIRALCEL OX (250 mm×30 mm, 10 μm); A: CO₂-ACN, B: EtOH (0.1% NH₃·H₂O); B %: 57%, isocratic elution mode] to afford two peaks.

N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-2H-1,2,3-triazole-2-carboxamide: Peak 1 was purified with prep-HPLC [column: Waters Xbridge150×25 mm×5 μm; A: water (0.1% FA), B: ACN; B %: 20%-50% over 9 min] and lyophilized to afford the title compound (6.15 mg, 1.62% yield) as an off-white solid; ¹H NMR (400 MHz, CDCl₃) δ=9.08 (br d, J=10.0 Hz, 1H), 8.24-8.07 (m, 1H), 7.72 (br s, 1H), 7.45 (br s, 1H), 7.28 (s, 2H), 7.04-7.03 (m, 1H), 6.97-6.74 (m, 1H), 5.44-5.23 (m, 1H), 4.43 (br d, J=9.6 Hz, 1H), 4.36-4.26 (m, 1H), 4.18-3.93 (m, 2H), 3.81 (br s, 2H), 3.55-3.39 (m, 6H), 3.31 (br s, 3H), 3.06 (br s, 1H), 2.51-2.36 (m, 1H), 2.35-2.08 (m, 4H), 2.01 (br s, 3H), 0.77 (br s, 3H); LCMS (ESI, M+1): m/z=676.3; SFC: 99% de, Column: Lux 3 μm Cellulose-4 50×4.6 mm I.D., 3 μm Mobile phase: Phase A for CO₂, and Phase B for EtOH (0.05% DEA); Gradient elution: EtOH (0.05% DEA) in CO₂ from 20% to 60%; Flow rate: 3 mL/min; Detector: PDA; Column Temp: 35C; Back Pressure: 100 Bar, T_{R}: 1.697 min

N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-1H-1,2,3-triazole-1-carboxamide: Peak 2 was purified with prep-HPLC [column: Waters Xbridge150×25 mm×5 μm; A: water (10 mM NH₄HCO₃), B: ACN, B %: 38%-68% over 9 min) and lyophilized to afford the title compound (4.45 mg, 1.27% yield) as an white solid; ¹H NMR (400 MHz, CDCl₃) δ=9.09 (br d, J=17.2 Hz, 1H), 7.78 (br s, 2H), 7.43 (dt, J=6.0, 9.2 Hz, 1H), 7.13 (dt, J=5.2, 9.2 Hz, 1H), 7.07-6.99 (m, 1H), 6.98-6.84 (m, 1H), 5.43-5.16 (m, 1H), 4.48-4.21 (m, 2H), 4.19-4.05 (m, 1H), 4.02-3.68 (m, 3H), 3.59-3.43 (m, 2H), 3.40-3.10 (m, 7H), 3.02 (br d, J=5.2 Hz, 1H), 2.50-2.37 (m, 1H), 2.35-2.04 (m, 4H), 2.03-1.86 (m, 3H), 0.78 (q, J=7.2 Hz, 3H); LCMS (ESI, M+1): m/z=676.3; SFC: 99% de, Column: Lux 3 μm Cellulose-4 50×4.6 mm I.D., 3 μm Mobile phase: Phase A for CO₂, and Phase B for EtOH (0.05% DEA); Gradient elution: EtOH (0.05% DEA) in CO₂ from 20% to 60%; Flow rate: 3 mL/min; Detector: PDA; Column Temp: 35C; Back Pressure: 100 Bar, T_{R}: 1.983 min

### Example 209

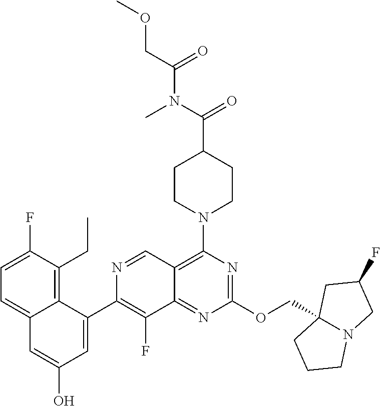 [SMILES: CCc1c(F)ccc2cc(O)cc(-c3ncc4c(N5CCC(C(=O)N(C)C(=O)COC)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-(2-methoxyacetyl)-N-methylpiperidine-4-carboxamide

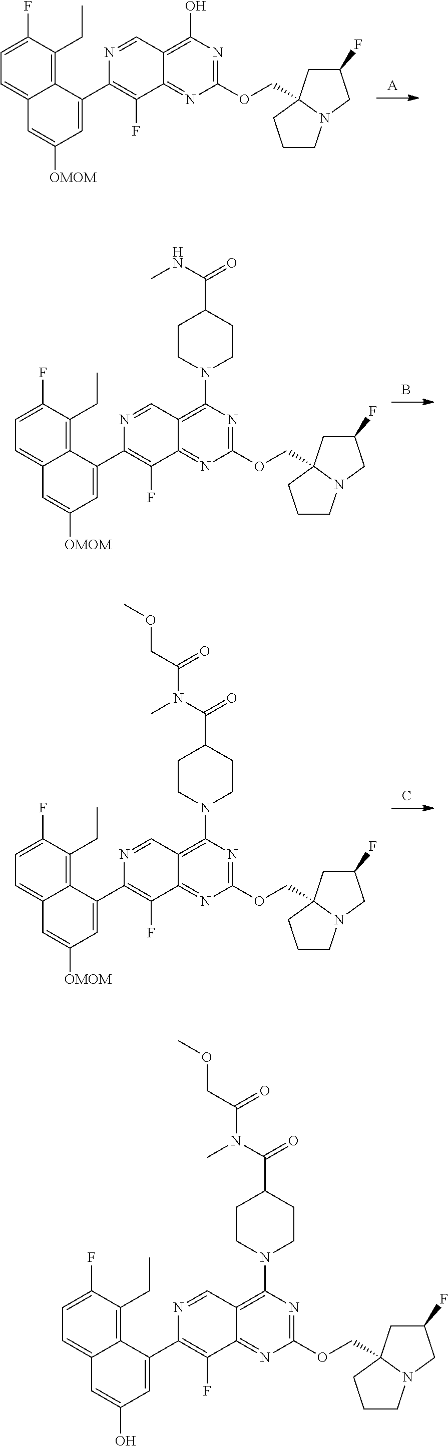

Step A. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (300 mg, 1.0 equiv), N-methylpiperidine-4-carboxamide (193 mg, 2.0 equiv, HCl) in DMF (3 mL) were added TEA (273 mg, 5.0 equiv) and PyBOP (422 mg, 1.5 equiv). The reaction was stirred at 25 °C for 1 hour. The mixture was filtered and purified by reversed phase flash chromatography [C18, 0.1% formic acid condition] to afford the title compound (300 mg, 82% yield) as white solid. LCMS (ESI, M+1): m/z=679.4.

Step B. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-(2-methoxyacetyl)-N-methylpiperidine-4-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide (100 mg, 1.0 equiv), 2-methoxyacetyl chloride (24.0 mg, 1.5 equiv) in 2-methyltetrahydrofuran (2 mL) was added DIEA (190.41 mg, 10 equiv). The reaction was stirred at 80 °C for 0.5 hours. The mixture was diluted with water (50 mL) and extracted with Ethyl acetate (2×20 mL). The combined organic layers were washed with brine (5 mL), dried over anhydrous sodium sulfate, concentrated and purified by reversed phase flash chromatography [C18, 0.1% formic acid condition] to afford the title compound (80 mg, 68% yield) as yellow solid; LCMS (ESI, M+1): m/z=751.4.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-(2-methoxyacetyl)-N-methylpiperidine-4-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-(2-methoxyacetyl)-N-methylpiperidine-4-carboxamide (80.0 mg, 1.0 equiv) in DCM (1 mL) was added TFA (1.54 g, 126 equiv) at 25 °C. The reaction was stirred at 25 °C for 1 hour. The mixture was concentrated under vacuum, diluted with water (10 mL), adjusted to pH=7 with saturated NaHCO₃ (10 mL) and extracted with ethyl acetate (2×10 mL). The combined organic layers were washed with brine (5 mL), dried over anhydrous sodium sulfate, concentrated and purified by reversed phase flash chromatography [C18, H₂O/ACN] to afford the title compound (7.83 mg, 10% yield) as white solid. ¹H NMR (400 MHz, CHLOROFORM-d) δ=8.92 (d, J=2.8 Hz, 1H), 7.53 (dd, J=5.6, 9.2 Hz, 1H), 7.22-7.12 (m, 2H), 7.06-6.88 (m, 1H), 5.53-5.13 (m, 1H), 4.55 (br d, J=13.2 Hz, 2H), 4.50-4.27 (m, 3H), 3.50 (s, 3H), 3.44-3.29 (m, 4H), 3.26 (s, 3H), 3.24-3.10 (m, 1H), 3.10-2.99 (m, 1H), 2.56-2.26 (m, 3H), 2.23-2.10 (m, 2H), 2.09-1.79 (m, 8H), 0.80 (br t, J=7.0 Hz, 3H); LCMS (ESI,M+1): m/z=707.4.

### Example 210

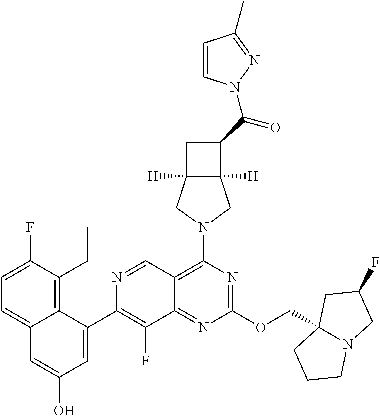 [SMILES: CCc1c(F)ccc2cc(O)cc(-c3ncc4c(N5C[C@H]6C[C@@H](C(=O)n7ccc(C)n7)[C@H]6C5)nc(OC[C@@]56CCCN5C[C@H](F)C6)nc4c3F)c12]

### ((1S,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone

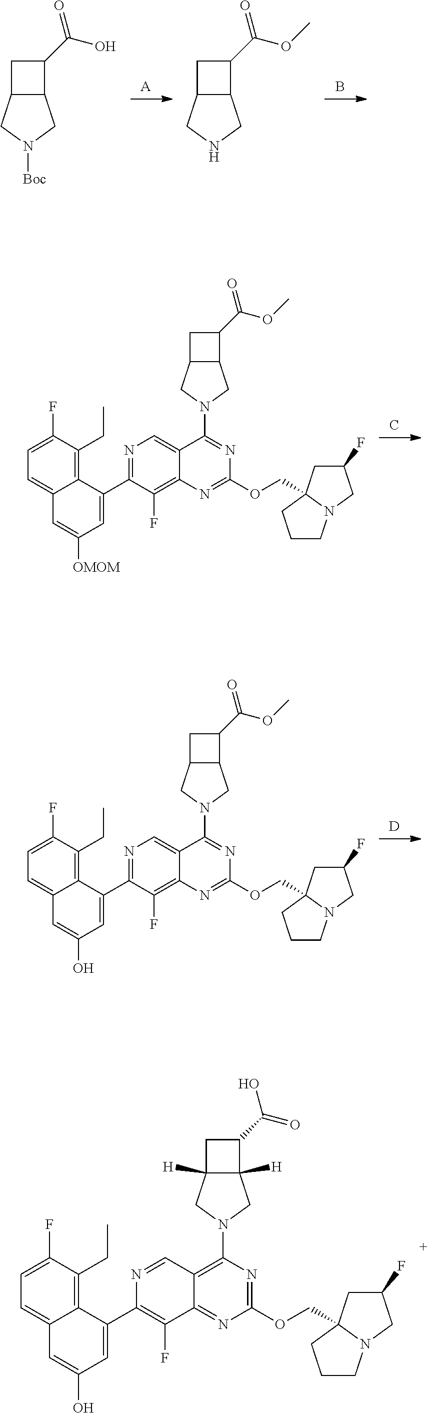

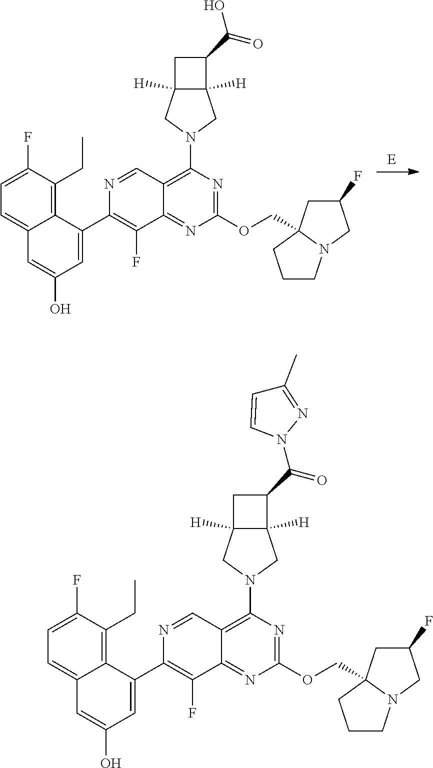

Step A. methyl 3-azabicyclo[3.2.0]heptane-6-carboxylate: A solution of 3-(tert-butoxycarbonyl)-3-azabicyclo[3.2.0]heptane-6-carboxylic acid (1.00 g, 1.0 equiv) in HCl·MeOH (2 M, 15 mL) was stirred at 20 °C for 1 hour. The mixture was concentrated to afford the title compound (1.00 g, crude, HCl) as yellow oil.

Step B. methyl 3-(7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.0]heptane-6-carboxylate: To a mixture of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (1.30 g, 1.0 equiv) and DIEA (1.51 g, 5.0 equiv) in DCM (20 mL) were added 2-nitrobenzenesulfonyl chloride (623 mg, 1.2 equiv) and DMAP (18.6 mg, 0.10 equiv). The reaction was stirred at 20 °C for 0.5 hours. Methyl 3-azabicyclo[3.2.0]heptane-6-carboxylate (484 mg, 1.1 equiv, HCl) was added into above mixture. The reaction was stirred at 20 °C for 0.1 hours. The mixture was diluted with water (30 mL) and extracted with DCM (3×20 mL). The combined organic layers were dried over anhydrous sodium sulfate, concentrated and purified with reversed phase flash chromatography [C18, 0.1% formic acid condition] to afford the title compound (1.50 g, 83% yield) as white solid; LCMS (ESI, M+1): m/z=692.4.

Step C. methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate: To a solution of methyl 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate (1.50 g, 1.0 equiv) in MeCN (5 mL) was added HCl·dioxane (2 M, 10 mL). The reaction was stirred at 0 °C for 0.5 hours. The mixture was concentrated. The residue was diluted with saturated Na₂CO₃ aqueous (20 mL) and extracted with DCM (3×15 mL). The combined organic layers were dried over anhydrous sodium sulfate, concentrated to afford the title compound (1.40 g, crude) as yellow solid; LCMS (ESI, M+1): m/z=648.4.

Step D. (1R,5R,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid and (1S,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid: To a solution of methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate (1.40 g, 1.0 equiv) in MeOH (3 mL) and THF (7 mL) was added a solution of LiOH·H₂O (272 mg, 3.0 equiv) in H₂O (3 mL). The reaction was stirred at 20 °C for 1 hour. The mixture was adjusted to pH≈7 with HCl (2 M). The mixture was purified with reversed phase flash chromatography [C18, 0.1% formic acid condition] and SFC [column: DAICEL CHIRALCEL OX (250 mm×30 mm, 10 μm); A: CO₂-ACN/MeOH (0.1% NH₃·H₂O); B %: 45%, isocratic elution mode] to afford two peaks.

Peak 1:(1R,5R,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (400 mg, 28% yield) as white solid; LCMS (ESI, M+1): m/z=634.3; SFC [Column: Chiralcel OX-3 50×4.6 mm I.D, 3 μm: Mobile phase: Phase A for CO₂ and Phase B for MeOH+ACN (0.05% DEA); Gradient elution: MeOH+ACN (0.05% DEA) in CO₂ from 30% to 60% Flow rate: 3 mL/min; Detector: PDA Column Temp: 35C; Back Pressure: 100Bar]. RT=1.131 mins.

Peak 2: (1S,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (440 mg, 31% yield) as white solid; LCMS (ESI, M+1): m/z=634.3; SFC [Column: Chiralcel OX-3 50×4.6 mm I.D, 3 μm: Mobile phase: Phase A for CO₂ and Phase B for MeOH+ACN (0.05% DEA); Gradient elution: MeOH+ACN (0.05% DEA) in CO₂ from 30% to 60% Flow rate: 3 mL/min; Detector: PDA Column Temp: 35C; Back Pressure: 100Bar]. RT=1.497 mins.

Step E. ((1S,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone: To a solution of (1S,5S,6R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (150 mg, 1.0 equiv) and DIEA (91.8 mg, 3.0 equiv) in DMF (2 mL) were added HATU (135 mg, 1.5 equiv) and 3-methyl-1H-pyrazole (58.3 mg, 3.0 equiv). The reaction was stirred at 20 °C for 12 hours. The mixture was filtered and purified with reversed phase flash [C18, neutral condition] to afford the title compound (8.44 mg, 5.0% yield) as off-white solid; ¹H NMR (400 MHz, DMSO-d₆) δ=9.97 (s, 1H), 9.18 (s, 1H), 8.21 (dd, J=2.4, 2.4 Hz, 1H), 7.78-7.74 (m, 1H), 7.40-7.26 (m, 2H), 7.02 (s, 1H), 6.45-6.44 (m, 1H), 5.39-5.14 (m, 1H), 4.39-4.19 (m, 2H), 4.16-3.95 (m, 4H), 3.95-3.87 (m, 1H), 3.58-3.56 (m, 1H), 3.14-2.97 (m, 3H), 2.88-2.77 (m, 1H), 2.45-2.10 (m, 8H), 2.06-1.95 (m, 3H), 1.89-1.67 (m, 3H), 0.76-0.69 (m, 3H); LCMS (ESI, M+1): m/z=698.4.

### Example 211

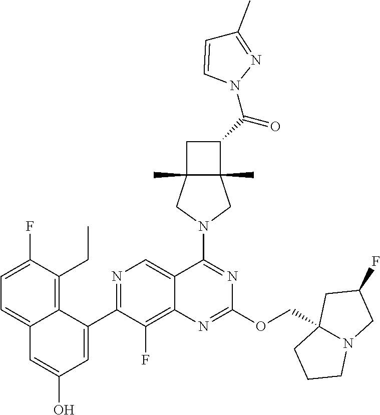 [SMILES: CCc1c(F)ccc2cc(O)cc(-c3ncc4c(N5C[C@]6(C)C[C@H](C(=O)n7ccc(C)n7)[C@]6(C)C5)nc(OC[C@@]56CCCN5C[C@H](F)C6)nc4c3F)c12]

### ((1R,5R,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptan-6-yl)(3-methyl-1H-pyrazol-1-yl)methanone

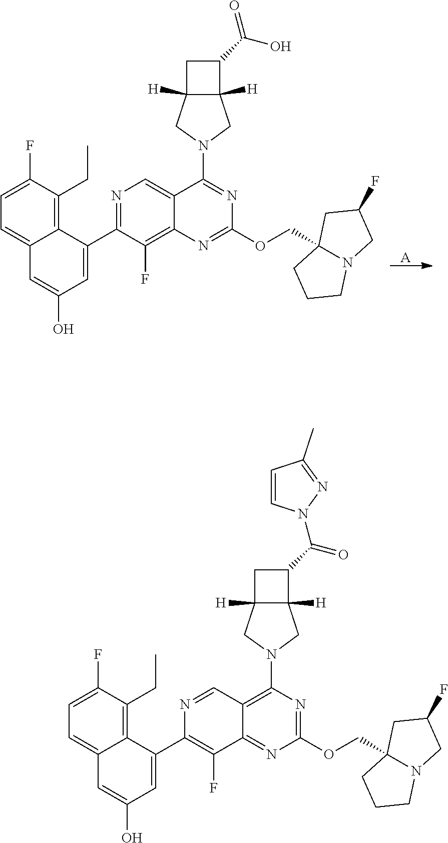

Step A. ((1R,5R,6S)-3-(7-(8-ethyl-7-fluoro-3-hydroxynaphthalen-1-yl)-8-fluoro-2-yl)-3-azabicyclo[3.2.0]heptan-6-yl)(3-methyl-1H-pyrazol-1-yl)methanone: To a solution of (1R,5R,6S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (150 mg, 1.0 equiv) and DIEA (91.8 mg, 3.0 equiv), in DMF (2 mL) were added HATU (135 mg, 1.5 equiv) and 3-methyl-1H-pyrazole (58.3 mg, 3.0 equiv). The reaction was stirred at 20 °C for 12 hours. The mixture was filtered and purified with reversed phase flash chromatography [C18, neutral condition] to afford the title compound (35.7 mg, 21% yield) as off-white solid; ¹H NMR (400 MHz, DMSO-d₆) δ=9.94 (br s, 1H), 9.18 (s, 1H), 8.21 (dd, J=2.4, 2.8 Hz, 1H), 7.78-7.74 (m, 1H), 7.40-7.26 (m, 2H), 7.01 (d, J=2.4 Hz, 1H), 6.45 (t, J=2.4 Hz, 1H), 5.39-5.14 (m, 1H), 4.38-4.20 (m, 2H), 4.11-3.92 (m, 4H), 3.90 (br s, 1H), 3.63-3.51 (m, 1H), 3.12-2.98 (m, 3H), 2.86-2.77 (m, 1H), 2.45-2.17 (m, 7H), 2.16-2.02 (m, 3H), 2.00-1.93 (m, 1H), 1.89-1.70 (m, 3H), 0.76-0.69 (m, 3H); LCMS (ESI, M+1): m/z=698.4.

### Example 212

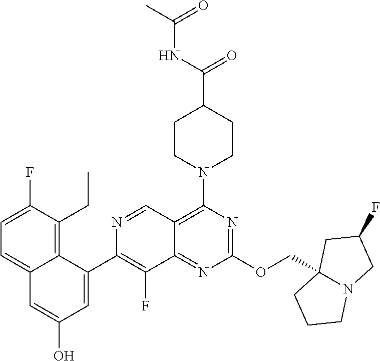 [SMILES: CCc1c(F)ccc2cc(O)cc(-c3ncc4c(N5CCC(C(=O)NC(C)=O)CC5)nc(OC[C@@]56CCCN5C[C@H](F)C6)nc4c3F)c12]

### N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxamide

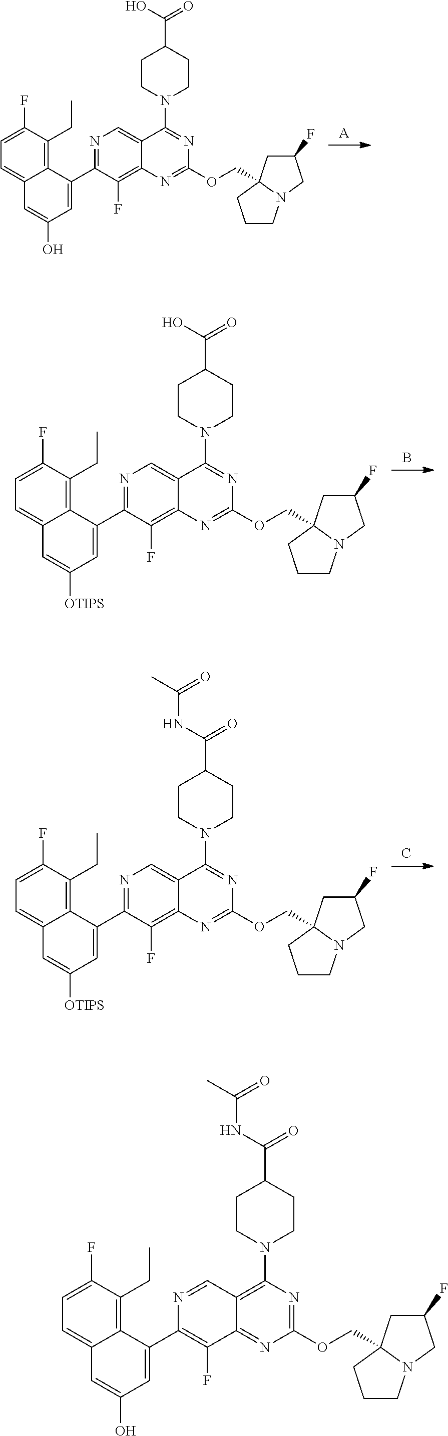

Step A. 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (500 mg, 1.0 equiv) and imidazole (821 mg, 15 equiv) in DMF (8.00 mL) were added TIPSCl (1.55 g, 10 equiv) and DMAP (49.1 mg, 0.5 equiv). The reaction was stirred at 40 °C for 16 hours. The mixture was quenched with H₂O (50 mL) and extracted with heptane (3×20 mL). The combined organic layers were dried over Na₂SO₄, filtered and concentrated to afford crude triisopropylsilyl 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylate. The crude was dissolved in MeOH (3.00 mL) and THF (3.00 mL) and then added aq. K₂CO₃ (2.0 equiv, 1 M). The reaction was stirred at 25 °C for 2 hours. The mixture was acidified with 1 M aq. HCl (20 mL) to pH 2-3 at 0 °C. The aqueous layer was extracted with Ethyl acetate (3×10 mL). The combined organic layers were dried over Na₂SO₄, concentrated and purified with reversed phase flash chromatography [C18, 0.1% formic acid condition] to afford the title compound (1.60 g, 81% yield) as yellow oil; ¹H NMR (400 MHz, DMSO-d6) δ=10.92-10.73 (m, 1H), 9.14 (s, 1H), 7.91 (dd, J=6.0, 9.2 Hz, 1H), 7.51 (d, J=2.8 Hz, 1H), 7.41 (t, J=9.2 Hz, 1H), 7.07 (d, J=2.4 Hz, 1H), 5.68-5.47 (m, 1H), 4.65-4.43 (m, 4H), 3.90-3.81 (m, 2H), 2.81-2.69 (m, 2H), 2.41-2.29 (m, 3H), 2.23-2.02 (m, 7H), 1.87-1.74 (m, 2H), 1.36-1.26 (m, 3H), 1.09 (s, 9H), 1.07 (s, 9H), 0.97 (s, 3H), 0.73 (br t, J=7.6 Hz, 3H); LCMS (ESI, M+1): m/z=778.4.

Step B. N-acetyl-1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxamide: To a solution of 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 equiv) and acetamide (50 mg, 6.6 equiv) in 2-methyltetrahydrofuran (1 mL) were added DIEA (166 mg, 10 equiv) and CMPI (131 mg, 4.0 equiv). The reaction was stirred at 80 °C for 2 hours. The mixture was diluted with water (5 mL) and extracted with ethyl acetate (3 mL). The organic layer was dried over sodium sulfate and concentrated to afford the title compound (105 mg, crude) as yellow oil.

Step C. N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxamide: To a solution of N-acetyl-1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxamide (100 mg, 1.0 equiv) in DMF (1.00 mL) was added CsF (185 mg, 10 equiv). The reaction was stirred at 25 °C for 0.5 hours. The mixture was filtered and purified with prep-HPLC [column: Waters Xbridge 150×25 mm×5 μm; A: water (NH₄HCO₃), B: ACN; B %: 35%-65% over 9 min] to afford the title compound (4.94 mg, 5.6% yield) as white solid; ¹H NMR (400 MHz, CHLOROFORM-d) δ=9.01-8.71 (m, 2H), 7.55-7.38 (m, 1H), 7.18-7.08 (m, 2H), 7.07-6.83 (m, 1H), 5.41-5.17 (m, 1H), 4.52-4.21 (m, 4H), 3.51-3.08 (m, 6H), 3.07-2.84 (m, 2H), 2.51-2.40 (m, 1H), 2.35 (s, 3H), 2.28-2.21 (m, 1H), 2.17-2.09 (m, 2H), 2.05-1.78 (m, 7H), 0.85-0.72 (m, 3H); LCMS (ESI, M+1): m/z=663.3.

### Example 213

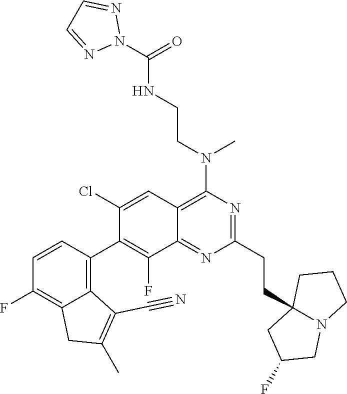 [SMILES: CC1=C(C#N)c2c(-c3c(Cl)cc4c(N(C)CCNC(=O)n5nccn5)nc(CC[C@@]56CCCN5C[C@H](F)C6)nc4c3F)ccc(F)c2C1]

### N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-2H-1,2,3-triazole-2-carboxamide

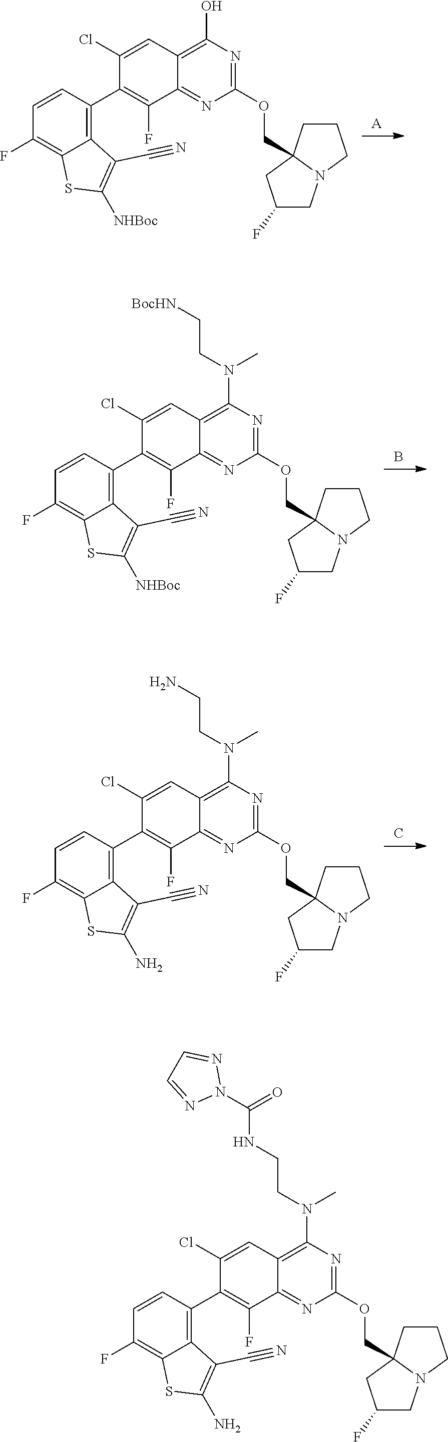

Step A. tert-butyl(4-(4-((2-((tert-butoxycarbonyl)amino)ethyl)(methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (450 mg, 1.0 equiv) in DMF (5 mL) were added PyBOP (435 mg, 1.2 equiv) and DIEA (270 mg, 3.0 equiv). The reaction was stirred at 25 °C for 20 minutes. tert-butyl(2-(methylamino)ethyl)carbamate (364 mg, 3.0 equiv) was added into the mixture. The reaction was stirred at 25 °C for 40 minutes. The mixture was filtered and purified by reversed-phase flash (0.1% NH₃·H₂O condition) to afford the title compound (520 mg, 90% yield) as white solid; LCMS (ESI, M+1): m/z=802.3.

Step B. 2-amino-4-(4-((2-aminoethyl)(methyl)amino)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl(4-(4-((2-((tert-butoxycarbonyl)amino)ethyl)(methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (450 mg, 1.0 equiv) in dichloromethane (5 mL) was added TFA (3.84 g, 60 equiv) at 0 °C. The reaction was stirred at 25 °C for 1 hour. The mixture was concentrated, dissolved with MeOH (5 mL), adjusted to pH=7 by using NaHCO₃solid, concentrated and purified by reversed-phase flash (0.1% FA condition) to afford the title compound (280 mg, 83% yield) as yellow solid; LCMS (ESI, M+1): m/z=602.3.

Step C. N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-2H-1,2,3-triazole-2-carboxamide: To a solution of 1H-1,2,3-triazole (20.0 mg, 1.0 equiv), DIEA (150 mg, 4.0 equiv) in dichloromethane (1 mL) was added bis(trichloromethyl) carbonate (40.0 mg, 0.47 equiv) at 0 °C. The reaction was stirred at 0 °C for 1 hour. 2-amino-4-(4-((2-aminoethyl)(methyl)amino)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (157 mg, 0.90 equiv) in THF (5 mL) was added dropwise at 0 °C. The reaction was stirred at 25 °C for 15 hours. The mixture was quenched with saturated NaHCO₃ aqueous (3 mL) and extracted with dichloromethane (3×2 mL). The combined organic layers were washed with brine (5 mL), dried over anhydrous sodium sulfate, concentrated and purified by prep-HPLC [Waters Xbridge 150×25 mm×5 μm; A: water (NH₄HCO₃); B %: ACN, B %: 40%-70% over 9 min], prep-chiral SFC [DAICEL CHIRALPAK AD (250 mm×30 mm, 10 μm); mobile phase: [CO₂-i-PrOH (0.1% NH₃·H₂O)]; B %: 40%, isocratic elution mode] and prep-HPLC [Phenomenex luna C18 150×25 mm×10 μm; A: water (FA); B %: ACN, B %: 18%-48% over 9 min] to afford the title compound (5.24 mg, 2.6% yield) as off-white solid; ¹H NMR (400 MHz, METHANOL-d₄) δ=8.55 (s, 1H), 8.12-7.84 (m, 2H), 7.73 (br s, 1H), 7.19 (dd, J=5.2, 8.4 Hz, 1H), 7.02 (t, J=8.8 Hz, 1H), 5.44-5.24 (m, 1H), 4.41-4.03 (m, 3H), 4.00-3.71 (m, 3H), 3.46 (br d, J=13.6 Hz, 1H), 3.35 (br s, 2H), 3.30-3.19 (m, 3H), 3.08 (dt, J=6.0, 9.6 Hz, 1H), 2.42-2.29 (m, 1H), 2.29-2.23 (m, 1H), 2.22-2.13 (m, 1H), 2.09-1.98 (m, 2H), 1.98-1.85 (m, 1H); LCMS (ESI, M+1): m/z=697.2.

### Example 214

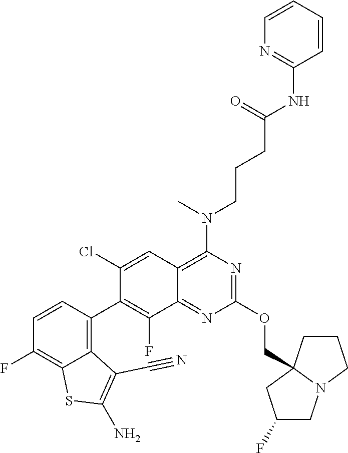 [SMILES: CN(CCCC(=O)Nc1ccccn1)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(pyridin-2-yl)butanamide

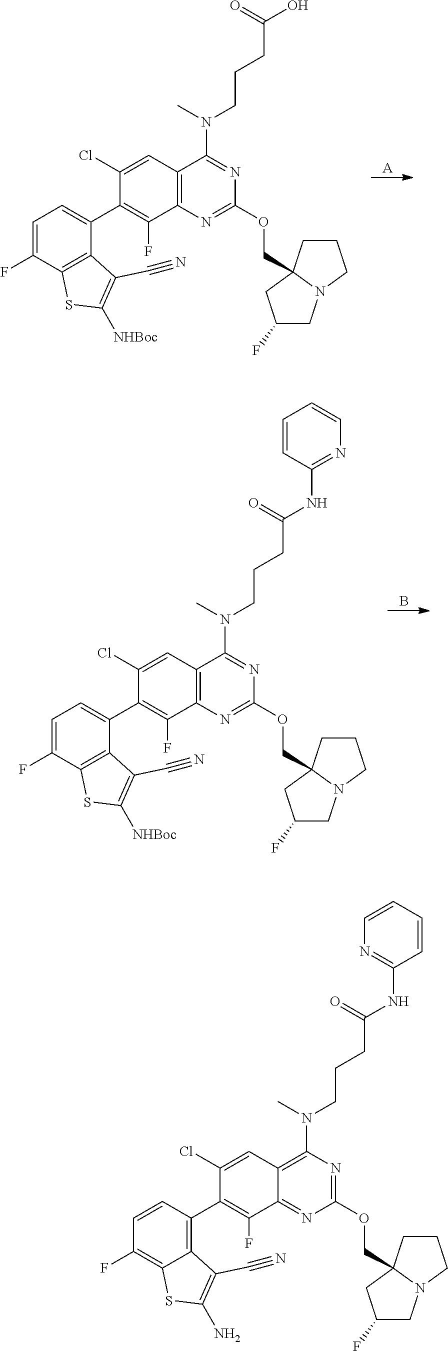

Step A. tert-butyl(4-(6-chloro-8-fluoro-2-(((2R,7aS)-2-fluorotetrahydro-1H-pyrrolizin-7a(5H)-yl)methoxy)-4-(methyl(4-oxo-4-(pyridin-2-ylamino)butyl)amino)quinazolin-7-yl)-3-cyano-7-fluorobenzo[b]thiophen-2-yl)carbamate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (100 mg, 1.0 equiv) and pyridin-2-amine (77.6 mg, 6.1 equiv) in 2-methyltetrahydrofuran (1.5 mL) were added DIEA (128 mg, 7.4 equiv) and CMPI (169 mg, 4.9 equiv). The reaction was stirred at 80 °C for 2 hours. The mixture was diluted with water (5 mL) and extracted with ethyl acetate (3 mL). The organic layer was dried over sodium sulfate and concentrated to afford the title compound (130 mg, crude) as yellow oil.

Step B. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(pyridin-2-yl) butanamide: To a solution of tert-butyl(4-(6-chloro-8-fluoro-2-(((2R,7aS)-2-fluorotetrahydro-1H-pyrrolizin-7a(5H)-yl)methoxy)-4-(methyl(4-oxo-4-(pyridin-2-ylamino)butyl)amino)quinazolin-7-yl)-3-cyano-7-fluorobenzo[b]thiophen-2-yl)carbamate (130 mg, 1.0 equiv) in DCM (0.5 mL) was added TFA (2.30 g, 127 equiv) at 25 °C. The reaction was stirred at 25 °C for 1 hour. The mixture was concentrated, basified with saturated NaHCO₃ solution (10 mL) and extracted with ethyl acetate (3×5 mL). The combined organic layers were dried over anhydrous sodium sulfate, concentrated and purified by prep-HPLC [column: Phenomenex luna C18 250×50 mm×15 μm; A: water (FA), B: ACN, B %: 15%-45% B over 65 min] to afford the title compound (41.2 mg, 36% yield) as white solid; ¹H NMR (400 MHz, CHLOROFORM-d) δ=9.08-8.92 (m, 1H), 8.20 (br d, J=4.8 Hz, 1H), 8.18-8.09 (m, 1H), 7.87 (s, 1H), 7.72-7.60 (m, 1H), 7.19-7.14 (m, 1H), 7.02-6.93 (m, 2H), 6.32-6.03 (m, 2H), 5.38-5.15 (m, 1H), 4.35-4.20 (m, 2H), 4.07-3.98 (m, 1H), 3.80-3.62 (m, 1H), 3.44 (br s, 1H), 3.41 (s, 3H), 3.38-3.17 (m, 2H), 3.06-2.99 (m, 1H), 2.48 (br t, J=6.8 Hz, 2H), 2.40-2.27 (m, 1H), 2.27-2.22 (m, 1H), 2.21-2.08 (m, 3H), 1.94 (br d, J=7.2 Hz, 3H); LCMS (ESI, M+1): m/z=721.3.

### Example 215

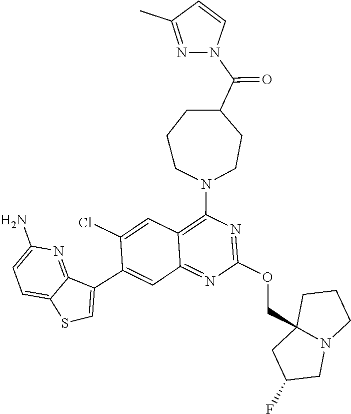 [SMILES: Cc1ccn(C(=O)C2CCCN(c3nc(OC[C@@]45CCCN4C[C@H](F)C5)nc4cc(-c5csc6ccc(N)nc56)c(Cl)cc34)CC2)n1]

### (1-(7-(5-aminothieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl) azepan-4-yl)(3-methyl-1H-pyrazol-1-yl)methanone

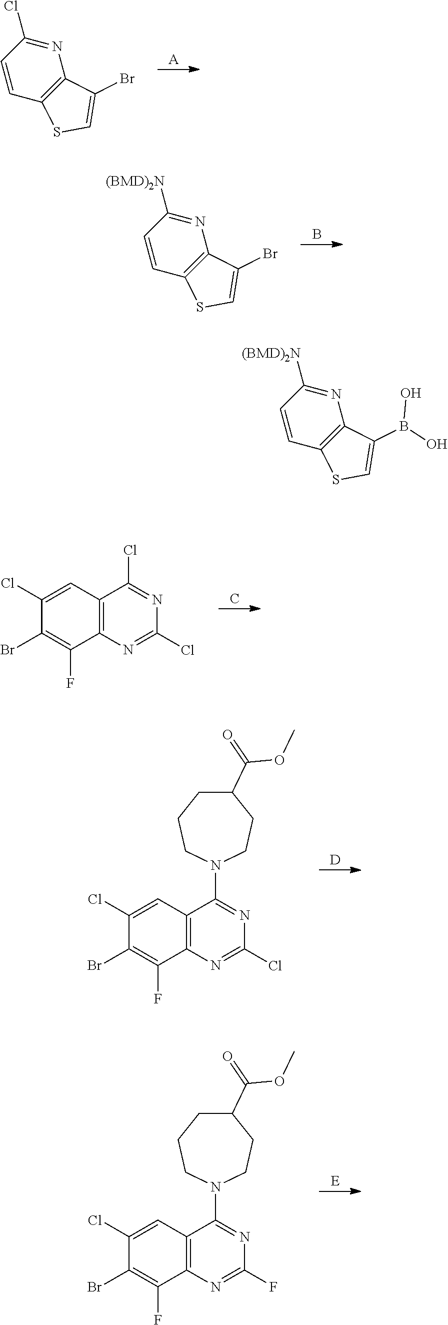

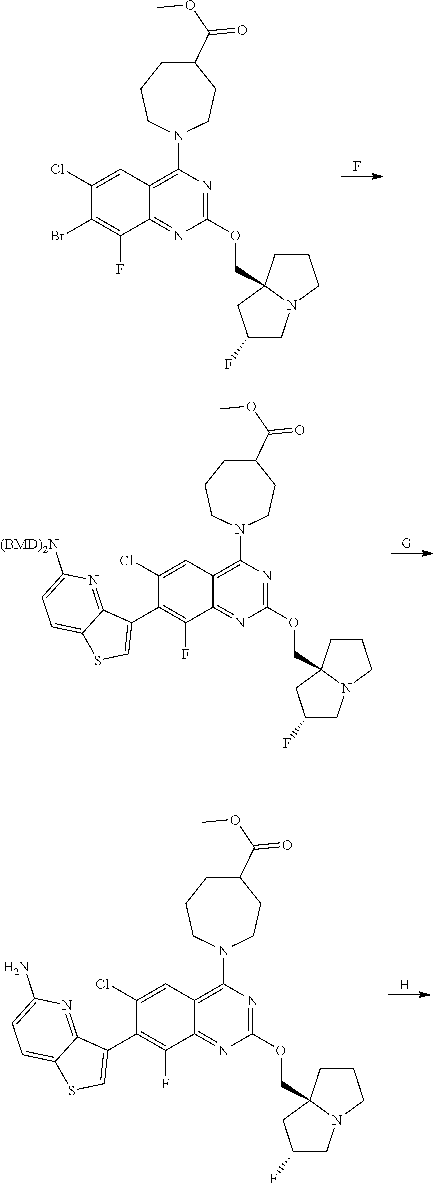

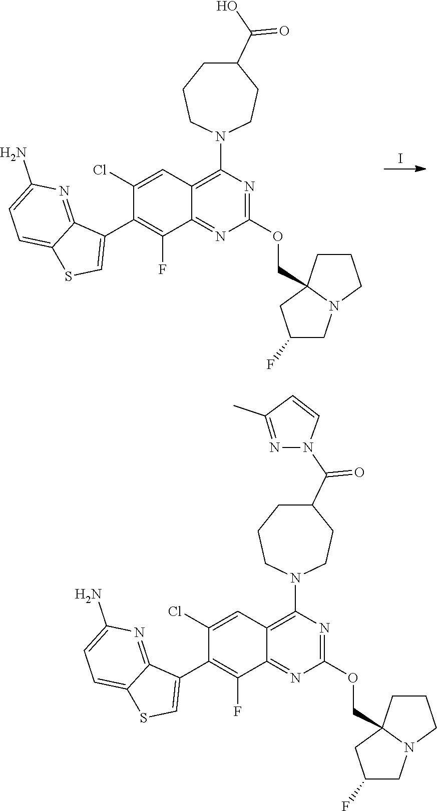

Step A. 3-bromo-N,N-bis(3,4-dimethylbenzyl)thieno[3,2-b]pyridin-5-amine; To a mixture of 3-bromo-5-chlorothieno[3,2-b]pyridine (2.00 g, 1.0 equiv) in DMF (20 mL) were added CsF (3.67 g, 3.0 equiv) and bis(2,4-dimethoxybenzyl)amine (25.5 g, 10.0 equiv). The reaction was stirred at 140 °C for 12 hours. The mixture was diluted with water (30 mL) and extracted with ethyl acetate (2×30 mL). The combined organic layers were washed with brine (4×50 mL), dried over anhydrous sodium sulfate, concentrated and purified with reversed phase flash [C18, 0.1% formic acid condition] to afford the title compound (1.60 g, 73% yield) as yellow solid; LCMS (ESI, M+1, M+3): m/z=529.0; 531.0.

Step B. (5-(bis(3,4-dimethylbenzyl)amino)thieno[3,2-b]pyridin-3-yl) boronic acid: To a mixture of 3-bromo-N,N-bis(3,4-dimethylbenzyl)thieno[3,2-b]pyridin-5-amine (1.50 g, 1.0 equiv) and 5,5,5′,5′-tetramethyl-2,2′-bi (1,3,2-dioxaborinane) (1.92 g, 3.0 equiv) in dioxane (20 mL) were added Pd(DPEphos)Cl₂ (203 mg, 0.1 equiv) and KOAc (834 mg, 3.0 equiv). The reaction was degassed and purged with nitrogen 3 times. The reaction was stirred at 80 °C for 0.5 hours. The mixture was diluted with water (20 mL) and extracted with ethyl acetate (2×20 mL). The combined organic layers were washed with brine (20 mL), dried over anhydrous sodium sulfate, concentrated and purified with reversed phase flash [C18, basic condition] to afford the title compound (300 mg, 21% yield) as yellow solid; LCMS (ESI, M+1): m/z=495.2.

Step C. methyl 1-(7-bromo-2,6-dichloro-8-fluoroquinazolin-4-yl)azepane-4-carboxylate: To a solution of 7-bromo-2,4,6-trichloro-8-fluoroquinazoline (10.0 g, 1.0 equiv) and DIEA (19.6 g, 5.0 equiv) in DCM (100 mL) was added methyl azepane-4-carboxylate (5.23 g, 1.1 equiv) at −40 °C. The reaction was stirred at −40 °C for 0.5 hours. The mixture was diluted with water (40 mL) and extracted with DCM (3×30 mL). The combined organic layers were dried over anhydrous sodium sulfate, concentrated and triturated with acetonitrile at 20 °C for 0.5 hours to afford the title compound (9.80 g, 72% yield) as yellow solid; LCMS (ESI, M+1, M+3): m/z=450.1, 452.1.

Step D. methyl 1-(7-bromo-6-chloro-2,8-difluoroquinazolin-4-yl)azepane-4-carboxylate: To a mixture of methyl 1-(7-bromo-2,6-dichloro-8-fluoroquinazolin-4-yl)azepane-4-carboxylate (8.80 g, 1.0 equiv) and 18-crown-6 ether (5.16 g, 1.0 equiv) in DMSO (88 mL) was added KF (11.3 g, 10.0 equiv). The reaction was stirred at 130 °C for 1 hour. The mixture was diluted with water (80 mL) and extracted with ethyl acetate (3×40 mL). The combined organic layers were washed with brine (3×120 mL), dried over anhydrous sodium sulfate, concentrated to afford the title compound (160 mg, crude) as yellow solid; LCMS (ESI, M+1, M+3): m/z=434.0, 436.0.

Step E. methyl 1-(7-bromo-6-chloro-8-fluoro-2-(((2R,7aS)-2-fluorotetrahydro-1H-pyrrolizin-7a(5H)-yl)methoxy)quinazolin-4-yl)azepane-4-carboxylate: To a mixture of ((2R,7aS)-2-fluorotetrahydro-1H-pyrrolizin-7a(5H)-yl)methanol (1.21 g, 1.2 equiv), t-BuONa (790 mg, 1.3 equiv) and 4 Å molecular sieve (500 mg) in THF (28 mL) was added methyl 1-(7-bromo-6-chloro-2,8-difluoroquinazolin-4-yl)azepane-4-carboxylate (2.75 g, 1.0 equiv) at 0 °C. The reaction was stirred at 0 °C for 2 hours. The mixture was concentrated. The residue was diluted with water (40 mL) and extracted with ethyl acetate (3×30 mL). The combined organic layers were dried over anhydrous sodium sulfate, concentrated and purified with reversed phase flash [C18, 0.1% formic acid condition] to afford the title compound (1.00 g, 24% yield) as yellow solid; LCMS (ESI, M+1, M+3): m/z=573.2, 575.2.

Step F. methyl 1-(7-(5-(bis(2,4-dimethoxybenzyl)amino)thieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a mixture of methyl 1-(7-bromo-6-chloro-8-fluoro-2-(((2R,7aS)-2-fluorotetrahydro-1H-pyrrolizin-7a(5H)-yl)methoxy)quinazolin-4-yl)azepane-4-carboxylate (300 mg, 1.0 equiv) and (5-(bis(3,4-dimethylbenzyl)amino)thieno[3,2-b]pyridin-3-yl) boronic acid (271 mg, 1.05 equiv) in dioxane (3 mL) were added Pd(dppf)Cl₂·CH₂Cl₂ (42.7 mg, 0.1 equiv) and K₃PO₄ (1.5 M, 1.05 mL, 3.0 equiv). The reaction was degassed and purged with nitrogen 3 times. The reaction was stirred at 90 °C for 0.5 hours. The mixture was diluted with water (20 mL) and extracted with ethyl acetate (2×20 mL). The combined organic layers were washed with brine (20 mL), dried over anhydrous sodium sulfate, concentrated and purified with reversed phase flash [C18, 0.1% formic acid condition] to afford the title compound (420 mg, 63% yield) as yellow solid; LCMS (ESI, M+1): m/z=943.4.

Step G. methyl 1-(7-(5-aminothieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl 1-(7-(5-(bis(2,4-dimethoxybenzyl)amino)thieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (400 mg, 1.0 equiv) in DCM (3 mL) was added TFA (5 mL) at 0 °C. The reaction was stirred at 0 °C for 1 hour. The mixture diluted with NaHCO₃ (10 mL) and extracted with DCM (2×10 mL). The combined organic layers were washed with brine (30 mL), dried over anhydrous sodium sulfate and concentrated to afford the title compound (410 mg, crude) as yellow oil.

Step H. 1-(7-(5-aminothieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a mixture of 1-(7-(5-aminothieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (400 mg, 1.0 equiv) in MeOH (3 mL) was added LiOH (2 M, 1.55 mL, 5 equiv). The reaction was stirred at 15 °C for 1 hour. The mixture was adjusted to pH=7 with HCl (1 M), concentrated and purified with reversed phase flash [C18, 0.1% formic acid condition] to afford the title compound (200 mg, 51% yield) as yellow solid; LCMS (ESI, M+1): m/z=629.3.

Step I. (1-(7-(5-aminothieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl) azepan-4-yl)(3-methyl-1H-pyrazol-1-yl)methanone: To a solution of 1-(7-(5-aminothieno[3,2-b]pyridin-3-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (80.0 mg, 1.0 equiv) and 3-methyl-1H-pyrazole (52.2 mg, 5.0 equiv) in DMF (0.5 mL) were added DIEA (82.2 mg, 5.0 equiv) and HATU (145 mg, 3.0 equiv). The reaction was stirred at 25 °C for 0.2 hours. The mixture was filtered and purified by prep-HPLC [column: Waters xbridge 150×25 mm 10 μm; mobile phase: A: water (ammonia hydroxide v/v), B: ACN; B %: 57%-87% over 10 min] and reversed phase flash [C18, neutral condition] to afford the title compound (13.05 mg, 14% yield) as yellow solid; ¹H NMR (400 MHz, DMSO-d6) δ=8.27 (s, 1H), 8.08-7.92 (m, 3H), 6.59 (d, J=8.8 Hz, 1H), 6.45 (d, J=2.8 Hz, 1H), 6.07 (br d, J=8.8 Hz, 2H), 5.36-5.18 (m, 1H), 4.24-4.16 (m, 2H), 4.12-4.02 (m, 2H), 3.93-3.79 (m, 3H), 3.07 (br d, J=7.2 Hz, 2H), 3.00 (br s, 1H), 2.85-2.78 (m, 1H), 2.28-2.23 (m, 1H), 2.25 (s, 2H), 2.19-2.08 (m, 3H), 2.06-1.96 (m, 4H), 1.95-1.60 (m, 5H); LCMS (ESI, M+1): m/z=693.3.

### Example 216

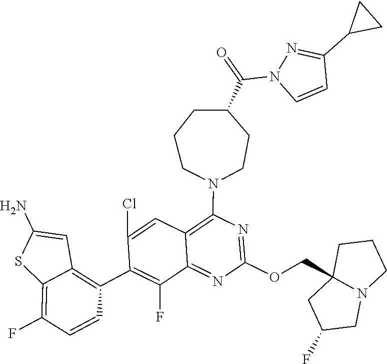

### 2-amino-4-(6-chloro-4-((S)-4-(3-cyclopropyl-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

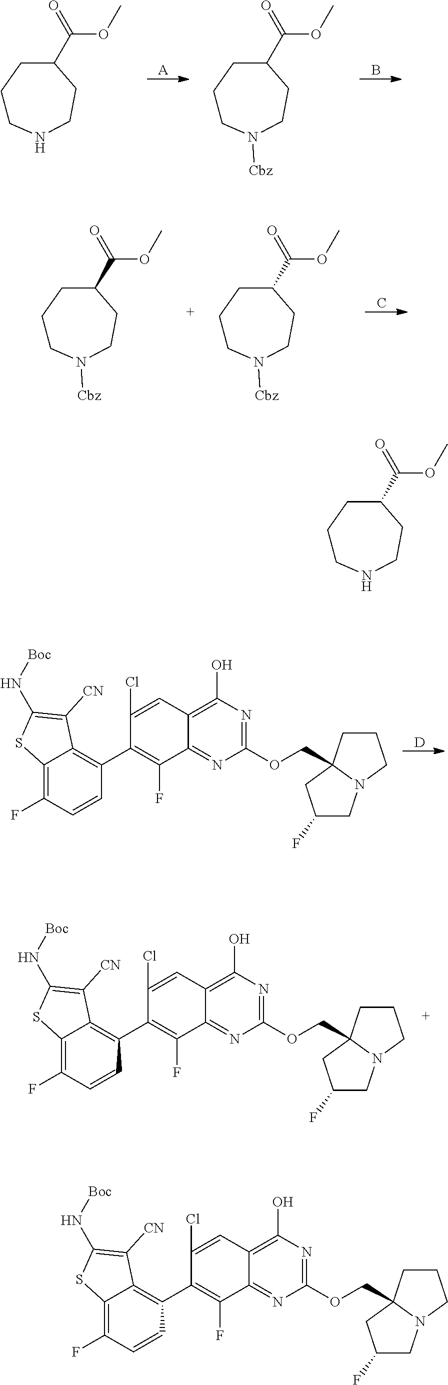

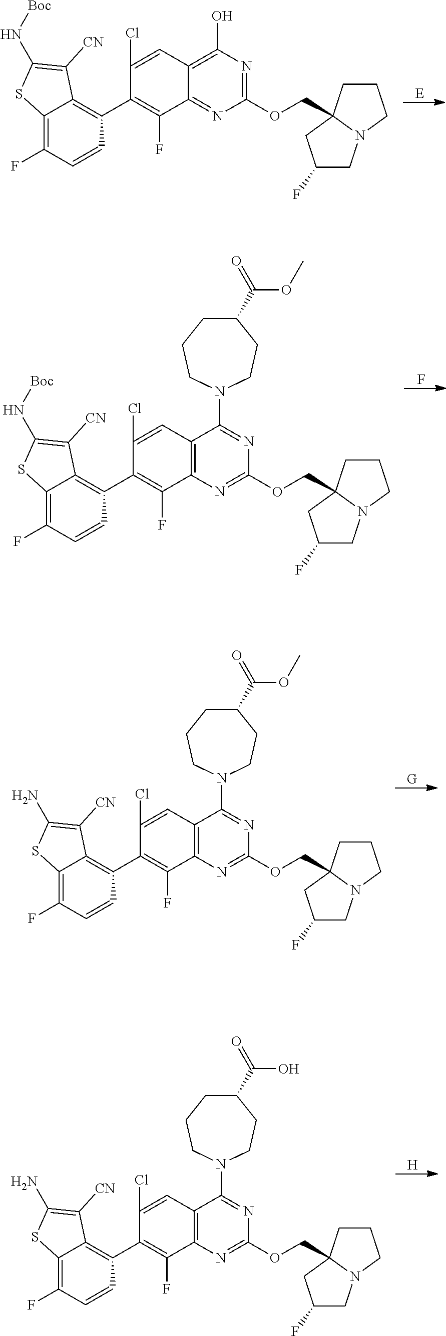

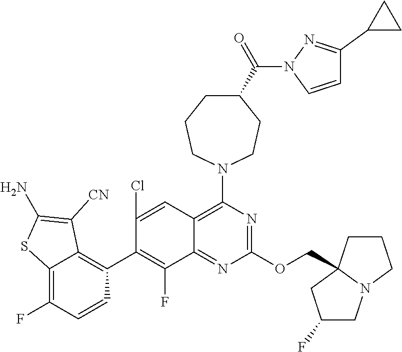

Step A 1-benzyl 4-methyl azepane-1,4-dicarboxylate: To a solution of methyl azepane-4-carboxylate hydrogen chloride (3.50 g, 1.0 equiv) in THF (25 mL) and H₂O (10 mL) were added NaHCO₃ (6.07 g, 4.0 equiv) and benzyl chloroformate (4.62 g, 1.5 equiv) at 0 °C. The reaction was stirred at 20 °C for 1 hour. The mixture was diluted with water (50 mL) and extracted with Ethyl acetate (4×20 mL). The combined organic layers were washed with brine (50 mL), dried over anhydrous sodium sulfate, concentrated and purified with reversed phase flash [water (0.1% formic acid)/acetonitrile] to afford the title compound (5.19 g, 96% yield) as colorless liquid; LCMS (ESI, M+1): m/z=292.2.

Step B 1-benzyl 4-methyl (R)-azepane-1,4-dicarboxylate and 1-benzyl 4-methyl (S)-azepane-1,4-dicarboxylate: 1-benzyl 4-methyl azepane-1,4-dicarboxylate (5.19 g) was separated with SFC [Column: Chiralpak AY-3×50×4.6 mm I.D., 3 μm; Mobile phase: Phase A for CO₂ and Phase B for IPA (0.05% DEA); Gradient elution: IPA (0.05% DEA) in CO₂ from 5% to 40%; Flow rate: 3 mL/minute; Detector: PDA; Column Temp: 35C; Back Pressure: 100Bar] to afford two peaks

Peak 1:1-benzyl 4-methyl (R)-azepane-1,4-dicarboxylate (2.48 g, 47% yield) as colorless liquid; LCMS (ESI, M+1): m/z=292.2; HPLC>98%, [Column: Chiralpak AY-3×50×4.6 mm I.D., 3 μm; Mobile phase: Phase A for CO₂ and Phase B for IPA (0.05% DEA); Gradient elution: IPA (0.05% DEA) in CO₂ from 5% to 40%; Flow rate: 3 mL/minute; Detector: PDA; Column Temp: 35 °C; Back Pressure: 100 Bar]. RT=1.195 minutes

Peak 2:1-benzyl 4-methyl (S)-azepane-1,4-dicarboxylate: (2.46 g, 47% yield) as colourless liquid; LCMS (ESI, M+1): m/z=292.2; HPLC>99%, [Column: Chiralpak AY-3×50×4.6 mm I.D., 3 μm; Mobile phase: Phase A for CO₂ and Phase B for IPA (0.05% DEA); Gradient elution: IPA (0.05% DEA) in CO₂ from 5% to 40%; Flow rate: 3 mL/minute; Detector: PDA; Column Temp: 35 °C; Back Pressure: 100 Bar]. RT=1.530 minutes

Step C. methyl (S)-azepane-4-carboxylate: To a solution of 1-benzyl 4-methyl (S)-azepane-1,4-dicarboxylate (2.0 g, 1.0 equiv) in MeOH (20 mL) was added Pd/C (500 mg, 10% purity) under N₂ atmosphere. The suspension was degassed and purged with H₂ for 3 times. The reaction was stirred at 20 °C for 1 hour under H₂ (15 psi) atmosphere. The mixture was filtered through a pad of Celite and concentrated to afford the title compound (1.26 g, 72% yield) as yellow oil; LCMS (ESI, M+1): m/z=158.3.

Step D. tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate and tert-butyl(4-((R)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate: tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (4.0 g, 1.0 equiv) was separated with SFC [Column: Chiralcel OX-3×50×4.6 mm I.D., 3 μm; Mobile phase: Phase A for CO₂ and Phase B for IPA+acetonitrile (0.05% DEA); Gradient elution: IPA+acetonitrile (0.05% DEA) in CO₂ from 20% to 60%; Flow rate: 3 mL/minute; Detector: PDA; Column Temp: 35 °C; Back Pressure: 100 Bar] to afford two peaks:

Peak 1: tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (2.10 g, 49% yield) as yellow solid; HPLC>99%, [Column: Chiralcel OX-3×50×4.6 mm I.D., 3 μm; Mobile phase: Phase A for CO₂ and Phase B for IPA+acetonitrile (0.05% DEA); Gradient elution: IPA+acetonitrile (0.05% DEA) in CO₂ from 20% to 60%; Flow rate: 3 mL/minute; Detector: PDA; Column Temp: 35 °C; Back Pressure: 100 Bar]; LCMS (ESI, M+1): m/z=646.2. RT=1.103 minutes

Peck 2: tert-butyl(4-((R)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (1.90 g, 47% yield) as yellow solid; HPLC>98%, [Column: Chiralcel OX-3×50×4.6 mm I.D., 3 μm; Mobile phase: Phase A for CO₂ and Phase B for IPA+acetonitrile (0.05% DEA); Gradient elution: IPA+acetonitrile (0.05% DEA) in CO₂ from 20% to 60%; Flow rate: 3 mL/minute; Detector: PDA; Column Temp: 35 °C; Back Pressure: 100 Bar]; LCMS (ESI, M+1): m/z=646.1. RT=2.123 minutes

Step E. 7-((R)-2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl 2-nitrobenzenesulfonate: To a solution of tert-butyl(4-((R)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (350 mg, 1.0 equiv) and DIEA (350 mg, 5.0 equiv) in DCM (3.5 mL) were added 2-nitrobenzenesulfonyl chloride (144 mg 1.2 equiv) and DMAP (6.62 mg, 0.1 equiv). The reaction was stirred at 20 °C for 0.5 hours. Methyl (S)-azepane-4-carboxylate (93.6 mg, 1.1 equiv) was added into above mixture. The reaction was stirred at 20 °C for 1 hour. The mixture was diluted with water (15 mL) and extracted with DCM (4×15 mL). The combined organic layers were washed with brine (30 mL), dried over anhydrous sodium sulfate, concentrated and purified with reversed phase flash chromatography [water (0.1% NH₃·H₂O)/acetonitrile=3/7] to afford the title compound (256 mg, 55% yield) as yellow solid; LCMS (ESI, M+1): m/z=785.2.

Step F. methyl (S)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of methyl (S)-1-((R)-7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (256 mg, 1.0 equiv) in DCM (2.5 mL) was added TFA (4 mL) at 0 °C. The reaction was stirred at 20 °C for 1 hour. The mixture was concentrated. The residue was diluted with Ethyl acetate (20 mL) and saturated Na₂CO₃ aqueous (20 mL) at 0 °C. The mixture was extracted with Ethyl acetate (4×15 mL). The combined organic layers were washed brine (20 mL), dried over anhydrous sodium sulfate and concentrated to afford the title compound (200 mg, 84% yield) as yellow solid; LCMS (ESI, M+1): m/z=685.3.

Step G. (S)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl (S)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (200 mg, 1.0 equiv) in MeOH (1.5 mL) and THF (0.5 mL) was added LiOH·H₂O (1.5 M in H₂O, 0.58 mL, 3.0 equiv). The reaction was stirred at 20 °C for 2 hours. The mixture was adjusted to pH=7 with 1 N HCl at 0 °C and purified with reversed phase flash chromatography [water (0.1% formic acid)/acetonitrile=3/2] to afford the title compound (140 mg, 70% yield) as yellow solid; LCMS (ESI, M+1): m/z=671.1.

Step H. (R)-2-amino-4-(6-chloro-4-((S)-4-(3-cyclopropyl-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of (S)-1-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (190 mg, 1.0 equiv) and 3-cyclopropyl-1H-pyrazole (95.2 mg, 3.0 equiv) in DMAc (2 mL) were added HATU (161 mg, 1.5 equiv) and DIEA (110 mg, 3.0 equiv). The reaction was stirred at 25 °C for 14 hours. The mixture was purified with prep-HPLC [column: Waters xbridge 150×25 mm×10 μm; mobile phase: water (NH₄HCO₃)-acetonitrile; gradient: 65%-85% B over 8 minutes] to afford the title compound (5.09 mg, 5% yield) as white solid; ¹H NMR (400 MHz, DMSO-d₆) δ=8.23 (d, J=2.8 Hz, 1H), 8.18-8.06 (m, 2H), 8.00 (s, 1H), 7.26-7.20 (m, 1H), 7.19-7.10 (m, 1H), 6.35 (d, J=2.8 Hz, 1H), 5.38-5.15 (m, 1H), 4.22 (br d, J=13.6 Hz, 2H), 4.11 (d, J=10.4 Hz, 1H), 4.00 (d, J=10.4 Hz, 1H), 3.96-3.87 (m, 1H), 3.84-3.74 (m, 1H), 3.74-3.64 (m, 1H), 3.13-3.01 (m, 2H), 3.00 (s, 1H), 2.87-2.76 (m, 1H), 2.35-2.24 (m, 1H), 2.22-1.91 (m, 9H), 1.88-1.72 (m, 3H), 1.70-1.59 (m, 1H), 1.01-0.89 (m, 2H), 0.79-0.65 (m, 2H); LCMS (ESI, M+1): m/z=761.3.

### Example 217

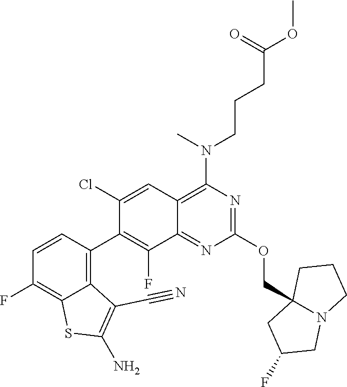 [SMILES: COC(=O)CCCN(C)c1nc(OC[C@@]23CCCN2C[C@H](F)C3)nc2c(F)c(-c3ccc(F)c4sc(N)c(C#N)c34)c(Cl)cc12]

### methyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate

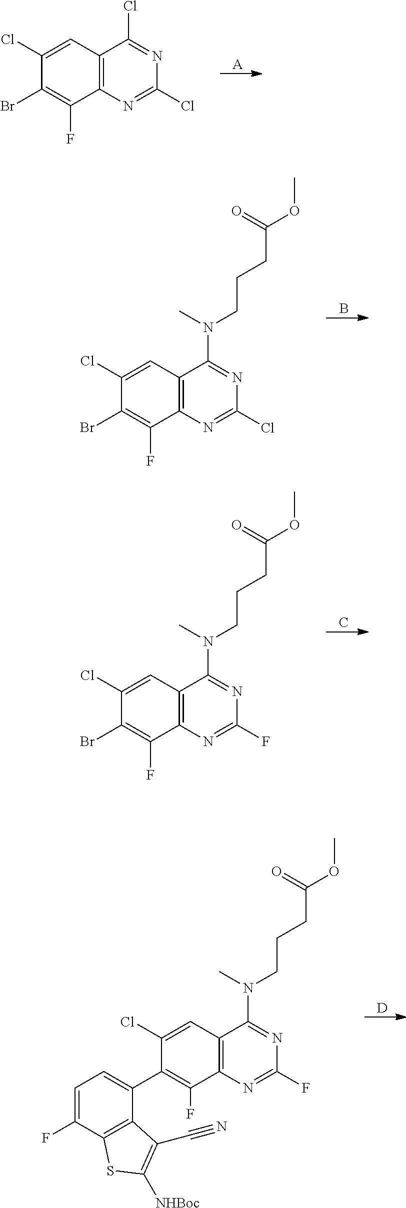

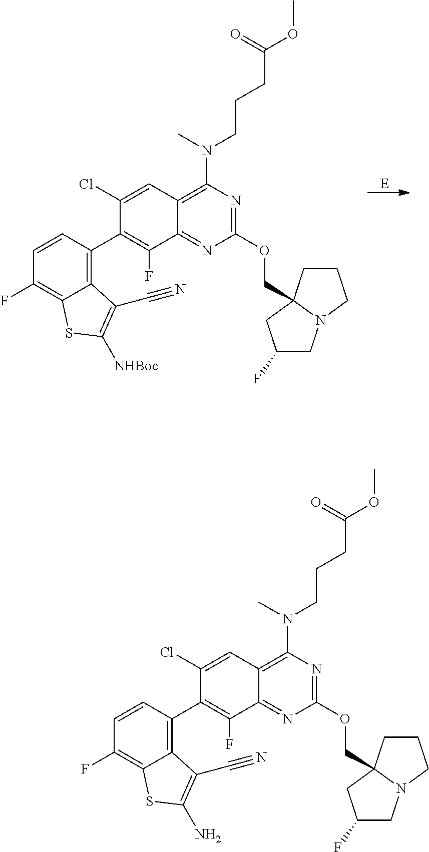

Step A. methyl 4-((7-bromo-2,6-dichloro-8-fluoroquinazolin-4-yl)(methyl)amino)butanoate: To a solution of 7-bromo-2,4,6-trichloro-8-fluoroquinazoline (2.0 g, 1.0 equiv) in DCM (30 mL) were added DIEA (3.91 g, 5.0 equiv) and methyl 4-(methylamino)butanoate hydrochloride (1.01 g, 1.0 equiv) at −40 °C. The reaction was stirred at −40 °C for 0.5 hours. The mixture was diluted with water (20 mL) and extracted with ethyl acetate (2×10 mL). The combined organic layers were dried over anhydrous sodium sulfate, concentrated and purified with column chromatography (SiO₂, Petroleum ether/Ethyl acetate=50/1 to 3/1) to afford the title compound (2.3 g, 89% yield) as a yellow solid.

Step B. Methyl 4-((7-bromo-6-chloro-2,8-difluoroquinazolin-4-yl)(methyl)amino)butanoate: To a solution of methyl 4-((7-bromo-2,6-dichloro-8-fluoroquinazolin-4-yl)(methyl)amino)butanoate (1.8 g, 1.0 equiv) in DMSO (20 mL) were added 1,4,7,10,13,16-hexaoxacyclooctadecane (112 mg, 0.10 equiv) and KF (2.46 g, 10 equiv). The reaction was stirred at 130 °C for 4 hours. The mixture diluted with water (40 mL) and extracted with ethyl acetate (2×30 mL). The combined organic layers were washed with brine (3×60 mL), dried over anhydrous sodium sulfate and concentrated to afford the title compound (1.6 g, crude) as a yellow solid.

Step C. methyl 4-((7-(2-((tert-butoxycarbonyl)amino)-3-cyano-7-fluorobenzo[b]thiophen-4-yl)-6-chloro-2,8-difluoroquinazolin-4-yl)(methyl)amino)butanoate: To a mixture of methyl 4-((7-bromo-6-chloro-2,8-difluoroquinazolin-4-yl)(methyl)amino)butanoate (1.6 g, 1.0 equiv) and tert-butyl N-[3-cyano-4-(5,5-dimethyl-1,3,2-dioxaborinan-2-yl)-7-fluoro-benzothiophen-2-yl]carbamate (2.38 g, 1.5 equiv) in THF (30 mL) were added Cs₂CO₃ (3.82 g, 3.0 equiv) and DPEPhosPdCl₂ (280 mg, 0.1.0 equiv). The reaction was degassed and purged with nitrogen 3 times. The reaction was stirred at 60 °C for 2 hours. The mixture diluted with water (30 mL) and extracted with ethyl acetate (3×25 mL). The combined organic layers were dried over anhydrous sodium sulfate, concentrated and purified by reversed phase flash [water (0.1% FA)/acetonitrile] to afford the title compound (1.67 g, 67% yield) as a yellow solid; ¹H NMR (400 MHz, CHLOROFORM-d) δ=8.05 (d, J=1.6 Hz, 1H), 7.78 (br s, 1H), 7.30 (dd, J=5.2, 8.4 Hz, 1H), 7.19-7.14 (m, 1H), 3.97-3.88 (m, 1H), 3.87-3.79 (m, 1H), 3.70 (s, 3H), 3.56 (s, 3H), 2.48 (t, J=7.2 Hz, 2H), 2.17 (quin, J=7.2 Hz, 2H), 1.58 (s, 9H); LCMS (ESI, M+1): m/z=620.1.

Step D. methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of ((2R,7aS)-2-fluorotetrahydro-1H-pyrrolizin-7a(5H)-yl)methanol (128 mg, 1.0 equiv) in THF (10 mL) was added 4 Å molecular sieve (200 mg, 1.0 equiv) and t-BuONa (155 mg, 2.0 equiv). The mixture was stirred at 15 °C for 10 minutes and then methyl 4-((7-(2-((tert-butoxycarbonyl)amino)-3-cyano-7-fluorobenzo[b]thiophen-4-yl)-6-chloro-2,8-difluoroquinazolin-4-yl)(methyl)amino)butanoate (500 mg, 1.0 equiv) was added. The reaction was stirred at 15 °C for 30 minutes. The mixture was added water (10 mL) and extracted with ethyl acetate (3×10 mL). The combined organic layers were dried over anhydrous sodium sulfate, concentrated and purified by reversed phase flash [water (0.1% FA)/acetonitrile] to afford the title compound (130 mg, 20% yield) as yellow solid; LCMS (ESI, M+1): m/z=759.2.

Step E. methyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of methyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate (70 mg, 1.0 equiv) in DCM (0.5 mL) was added TFA (767 mg, 73 equiv). The reaction was stirred at 15 °C for 0.5 hours. The mixture was concentrated in vacuum and basified with saturated aqueous NaHCO₃ (2 mL). The aqueous phase was extracted with ethyl acetate (2×2 mL). The combined organic layers were washed with NaHCO₃ (3×3 mL), dried over anhydrous sodium sulfate, concentrated and purified with prep-HPLC [column: Phenomenex luna C18 150×25 mm×10 μm; A: water (FA), B: ACN, B %: 20%-50% over 9 min] to afford the title compound (26.7 mg, 44% yield) as white solid; ¹H NMR (400 MHz, METHANOL-d₄) δ=8.11-8.04 (m, 1H), 7.20 (dd, J=5.2, 8.4 Hz, 1H), 7.03 (dd, J=8.8, 9.6 Hz, 1H), 5.42-5.22 (m, 1H), 4.34-4.20 (m, 2H), 3.94-3.81 (m, 2H), 3.65 (s, 3H), 3.52 (s, 3H), 3.40-3.34 (m, 1H), 3.28-3.17 (m, 2H), 3.08-2.98 (m, 1H), 2.48 (t, J=7.2 Hz, 2H), 2.43-2.09 (m, 5H), 2.06-1.84 (m, 3H); LCMS (ESI, M+1): m/z=659.2.

### Example 218

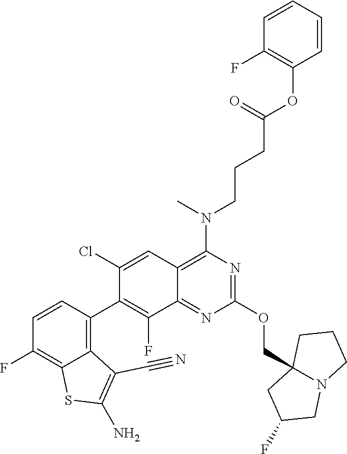 [SMILES: CN(CCCC(=O)Oc1ccccc1F)c1nc(OC[C@@]23CCCN2C[C@H](F)C3)nc2c(F)c(-c3ccc(F)c4sc(N)c(C#N)c34)c(Cl)cc12]

### 2-fluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate

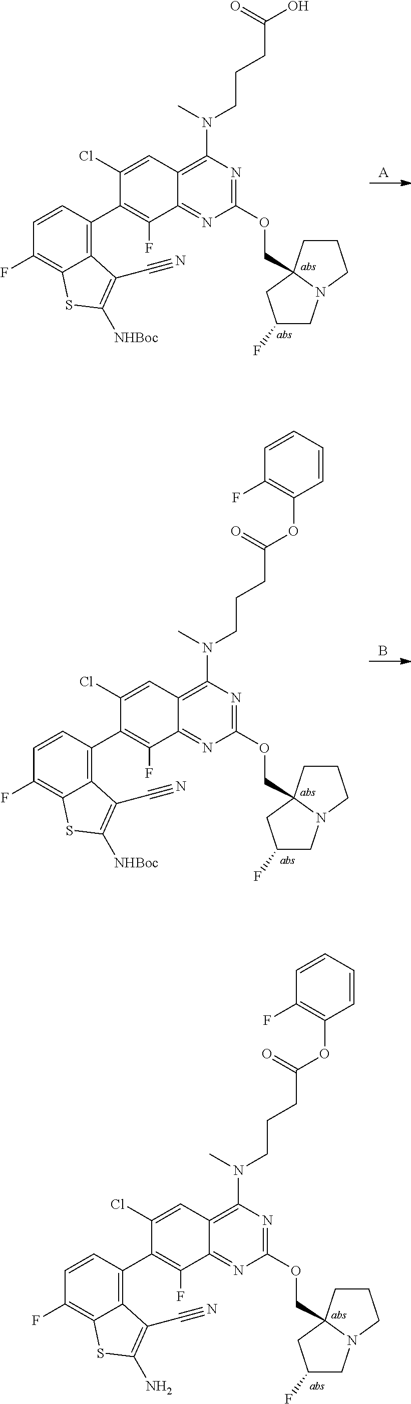

Step A. 2-fluorophenyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (100 mg, 1.0 equiv) and 2-fluorophenol (45.1 mg, 3.0 equiv) in DCM (1 mL) were added DMAP (49.2 mg, 3.0 equiv) and EDCI (77.2 mg, 1.5 equiv). The reaction was stirred at 25 °C for 10 hours. The mixture was concentrated and purified with reversed phase flash chromatography [C18, 0.1% formic acid condition] to afford the title compound (70 mg, 58% yield) as white solid. LCMS (ESI, M+1): m/z=839.2.

Step B. 2-fluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of 2-fluorophenyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate (65.0 mg, 1.0 equiv) in DCM (1 mL) was added TFA (1.54 g, 174 equiv) at 25 °C. The reaction was stirred at 25 °C for 0.5 hours. The mixture was concentrated, diluted with water (10 mL), adjusted to pH=7 with saturated NaHCO₃ (10 mL) and extracted with ethyl acetate (2×10 mL). The combined organic layers were washed with brine (5 mL), dried over anhydrous sodium sulfate, concentrated, and purified with prep-HPLC [Waters Xbridge 150×25 mm×5 μm; A: water (ammonia hydroxide v/v); B: ACN; B %: 50%-80% over 10 min] to afford the title compound (10.1 mg, 17% yield) as white solid. ¹HNMR (400 MHz, CHLOROFORM-d) δ=7.91 (s, 1H), 7.20 (br dd, J=3.6, 7.6 Hz, 2H), 7.17 (s, 1H), 7.16-7.08 (m, 2H), 7.02 (t, J=8.8 Hz, 1H), 5.73-5.55 (m, 2H), 5.43-5.13 (m, 1H), 4.35-4.14 (m, 2H), 4.07-3.75 (m, 2H), 3.47 (s, 3H), 3.38-3.12 (m, 3H), 3.06-2.90 (m, 1H), 2.76 (t, J=7.2 Hz, 2H), 2.34-2.13 (m, 5H), 1.91 (br d, J=7.6 Hz, 3H); LCMS (ESI,M+1): m/z=739.1.

### Example 219

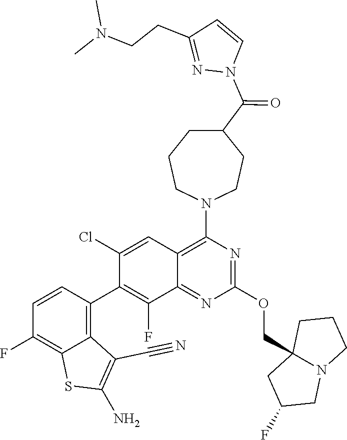 [SMILES: CN(C)CCc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(6-chloro-4-(4-(3-(2-(dimethylamino)ethyl)-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

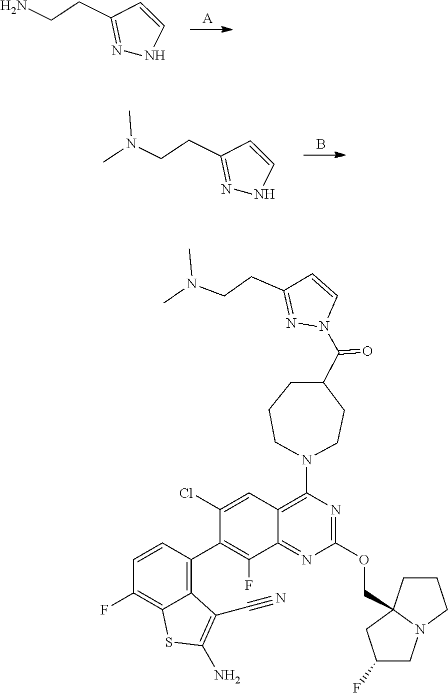

Step A. N,N-dimethyl-2-(1H-pyrazol-3-yl) ethan-1-amine; A mixture of 2-(1H-pyrazol-3-yl) ethan-1-amine 2 hydrogen chloride (1.0 g, 1.0 equiv) and HCHO (5.45 g, 37% purity, 12.4 equiv) aqueous in MeOH (10 mL) was stirred at 25 °C for 1 hour. NaBH₃CN (683 mg, 2.0 equiv) was added into above mixture at 0 °C under N₂ atmosphere. The reaction was stirred at 25 °C for 24 hours and at 45 °C for 8 hours. The mixture was quenched with saturated K₂CO₃ aqueous (1 mL), stirred at 25 °C for 1 hour and purified with reversed phase flash [water (NH₃·H₂O, 0.1%)/acetonitrile=13/7] to afford the title compound (0.45 g, 59% yield) as light yellow oil; LCMS (ESI, M+1): m/z=140.2.

Step B. 2-amino-4-(6-chloro-4-(4-(3-(2-(dimethylamino)ethyl)-1H-pyrazole-1-carbonyl) azepan-1-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (45.0 mg, 1.0 equiv) and N,N-dimethyl-2-(1H-pyrazol-3-yl) ethan-1-amine (28.0 mg, 3.0 equiv) in DMAc (0.4 mL) were added DIEA (26.0 mg, 3.0 equiv) and HATU (51 mg, 2.0 equiv). The reaction was stirred at 20 °C for 14 hours. The mixture was purified with reversed phase flash chromatography [water (0.1% formic acid)/acetonitrile=7/3] to afford the title compound (11.3 mg, 20% yield, 0.15 HCOOH) as white solid; ¹H NMR (400 MHz, DMSO-d6) δ=8.27 (d, J=2.4 Hz, 1H), 8.10 (br s, 2H), 7.99 (br d, J=1.6 Hz, 1H), 7.29-7.19 (m, 1H), 7.18-7.11 (m, 1H), 6.52 (t, J=2.6 Hz, 1H), 5.26 (d, J=54.4 Hz, 1H), 4.24-3.73 (m, 7H), 3.11-2.96 (m, 3H), 2.86-2.71 (m, 3H), 2.57-2.50 (m, 2H), 2.30-2.25 (m, 1H), 2.23-1.96 (m, 13H), 1.86-1.61 (m, 4H); LCMS (ESI, M+1): m/z=792.3.

### Example 220

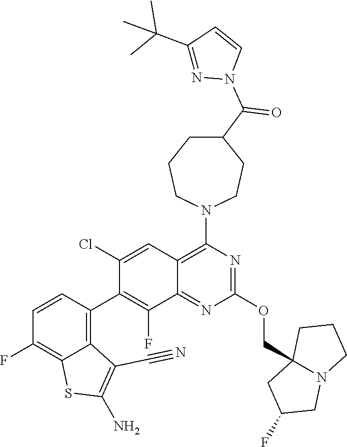 [SMILES: CC(C)(C)c1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1]

### 2-amino-4-(4-(4-(3-(tert-butyl)-1H-pyrazole-1-carbonyl) azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

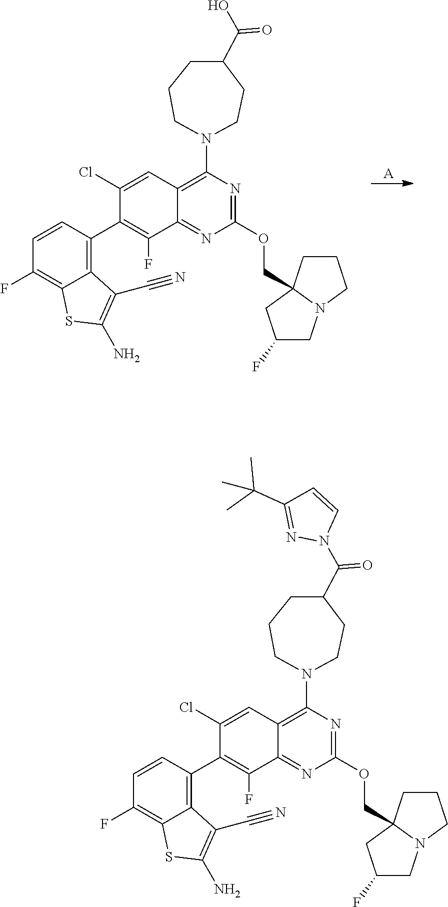

Step A. 2-amino-4-(4-(4-(3-(tert-butyl)-1H-pyrazole-1-carbonyl) azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (100 mg, 1.0 equiv), EDCI (42.8 mg, 1.5 equiv) and HOBt (30.2 mg, 1.5 equiv) in DMF (0.5 mL) were added 3-(tert-butyl)-1H-pyrazole (37.0 mg, 2.0 equiv) and and DIEA (57.8 mg, 3.0 equiv). The reaction was stirred at 20 °C for 12 hours. The mixture was filtered and purified by prep-HPLC [Phenomenex luna C18 150×25 mm×10 μm; A: water (FA), B: ACN; B %: 59%-79% B over 10 min] to afford the title compound (19.3 mg, 16% yield) as off-white solid; ¹H NMR (400 MHz, DMSO-d₆) δ=8.31-8.21 (m, 1H), 8.20-7.92 (m, 3H), 7.34-7.08 (m, 2H), 6.65-6.55 (m, 1H), 5.47-5.20 (m, 1H), 4.41-3.55 (m, 8H), 3.16-2.86 (m, 2H), 2.36-1.77 (m, 12H), 1.75-1.63 (m, 1H), 1.30-1.19 (m, 9H); LCMS (ESI, M+1): m/z=777.4.

### Example 221

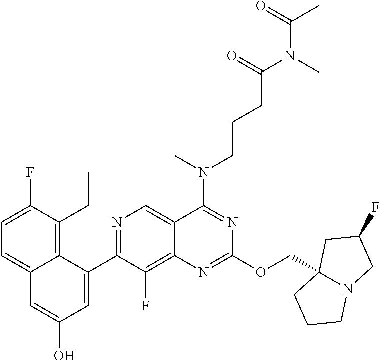 [SMILES: CCc1c(F)ccc2cc(O)cc(-c3ncc4c(N(C)CCCC(=O)N(C)C(C)=O)nc(OC[C@@]56CCCN5C[C@H](F)C6)nc4c3F)c12]

### N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylbutanamide

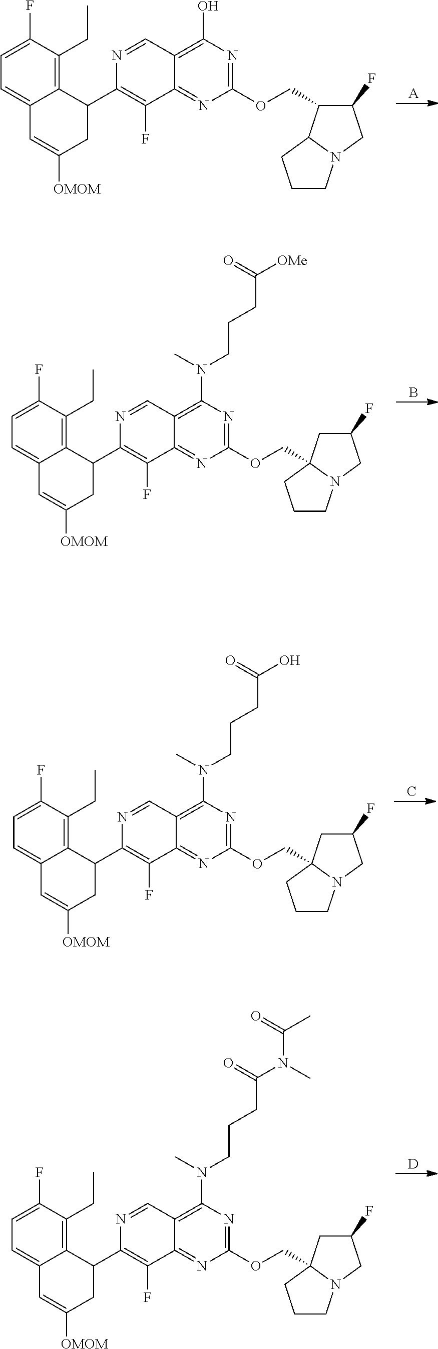

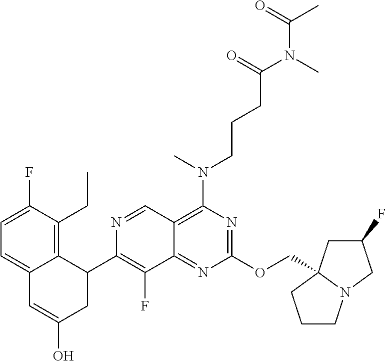 [SMILES: CCc1c(F)ccc2c1C(c1ncc3c(N(C)CCCC(=O)N(C)C(C)=O)nc(OC[C@@]45CCCN4C[C@H](F)C5)nc3c1F)CC(O)=C2]

Step A. methyl 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoate: To a solution of 7 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 equiv) in DMF (5 mL) were added DIEA (349 mg, 3.0 equiv) and PyBOP (563 mg, 1.2 equiv) slowly at 25 °C, the reaction was stirred at 25 °C for 20 minutes. And then methyl 4-(methylamino)butanoate (142 mg, 1.2 equiv, HCl) was added at 25 °C slowly. The reaction was stirred at 25 °C for 1 hour. The mixture was filtered and purified by reversed-phase flash (0.1% FA condition) to afford the title compound (400 mg, 61% yield, HCOOH) as off-white solid; LCMS (ESI, M+1): m/z=668.3.

Step B. 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoic acid: A solution of ethyl methyl methyl 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoate (400 mg, 1.0 equiv, HCOOH) in MeOH (12 mL) was added LiOH·H₂O (2 M, 12 mL, 43 equiv). The reaction was stirred at 40 °C for 0.5 hours. The mixture was filtered and purified by reversed-phase flash (0.1% FA condition) to afford the title compound (240 mg, 60% yield, HCOOH) as white solid; LCMS (ESI, M+1): m/z=654.4.

Step C. N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylbutanamide: To a solution of 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanoic acid (120 mg, 1.0 equiv, HCOOH) and N-methylacetamide (50.0 mg, 4.0 equiv) in THF (2 mL) were added DIEA (66.5 mg, 3.0 equiv) and 2-chloro-1-methylpyridin-1-ium iodide (219 mg, 5.0 equiv). The reaction was stirred at 25 °C for 12 hours. The mixture was filtered and purified by prep-HPLC [Phenomenex luna C18 150×40 mm×15 μm; A: water (FA), B: ACN, B %: 20%-50% over 15 min] to afford the title compound (30.0 mg, 22% yield, HCOOH) as light yellow solid; LCMS (ESI, M+1): m/z=709.3.

Step D. N-acetyl-4-((7-(8-ethyl-7-fluoro-3-hydroxynaphthalen-1-yl)-8-fluoro-2-yl)(methyl)amino)-N-methylbutanamide: To a solution of N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylbutanamide (30.0 mg, 1.0 equiv, HCOOH) in dichloromethane (1.0 mL) was added TFA (767 mg, 169 equiv) at 0 °C. The reaction was stirred at 25 °C for 1 hour. The mixture was concentrated, diluted with MeOH (3 mL), adjusted to pH about 7 using NaHCO₃solid, filtered, concentrated and purified by prep-HPLC [Waters Xbridge Prep OBD C18 150×25 mm×10 μm; A: water (FA), B: ACN, B %: 15%-45% over 9 min] and prep-chiral SFC [DAICEL CHIRALPAK AD (250×30 mm, 10 μm); mobile phase: [CO₂-ACN/i-PrOH (0.1% NH₃H₂O)]; B %: 40%, isocratic elution mode] to afford the title compound (10.5 mg, 38%, yield) as yellow solid; ¹H NMR (400 MHz, CHLOROFORM-d) δ=0.81 (br t, J=7.20 Hz, 3H), 2.07-2.21 (m, 6H), 2.25-2.35 (m, 2H), 2.37 (s, 3H), 2.44-2.56 (m, 2H), 2.85-2.95 (m, 2H), 3.02-3.14 (m, 1H), 3.19 (s, 3H), 3.25-3.41 (m, 1H), 3.48 (br d, J=3.04 Hz, 3H), 3.58-3.90 (m, 4H), 4.37 (br s, 1H), 4.59 (br dd, J=4.52, 1.84 Hz, 1H), 5.26-5.48 (m, 1H), 7.03-7.23 (m, 3H), 7.49-7.57 (m, 1H), 9.05-9.16 (m, 1H); LCMS (ESI, M+1): m/z=665.3.

### Example 222

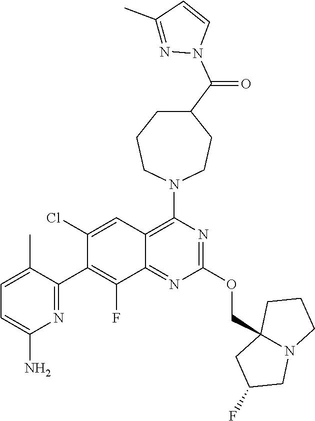 [SMILES: Cc1ccn(C(=O)C2CCCN(c3nc(OC[C@@]45CCCN4C[C@H](F)C5)nc4c(F)c(-c5nc(N)ccc5C)c(Cl)cc34)CC2)n1]

### (1-(7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-yl) azepan-4-yl)(3-methyl-1H-pyrazol-1-yl)methanone

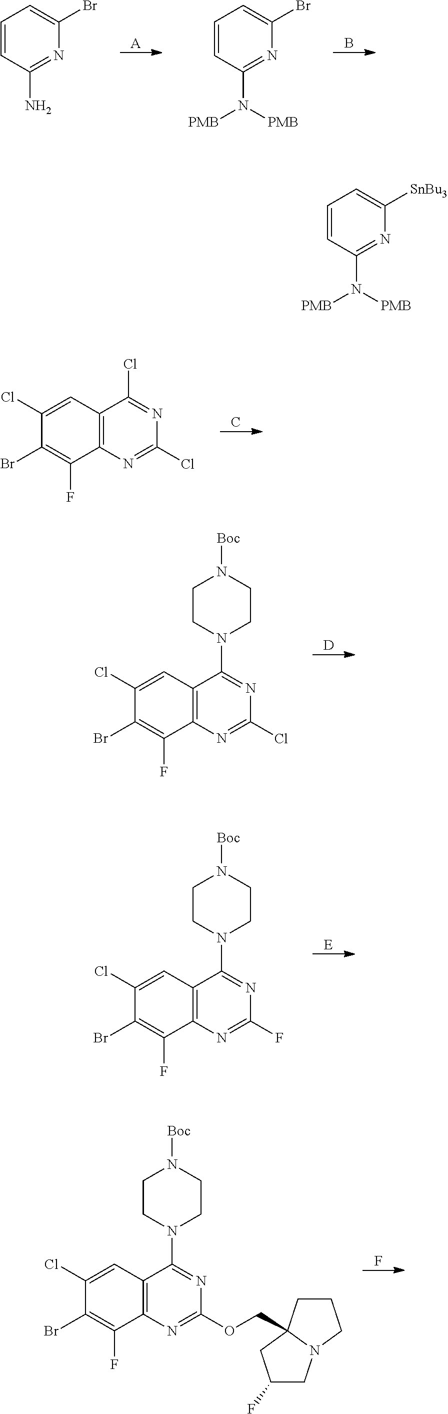

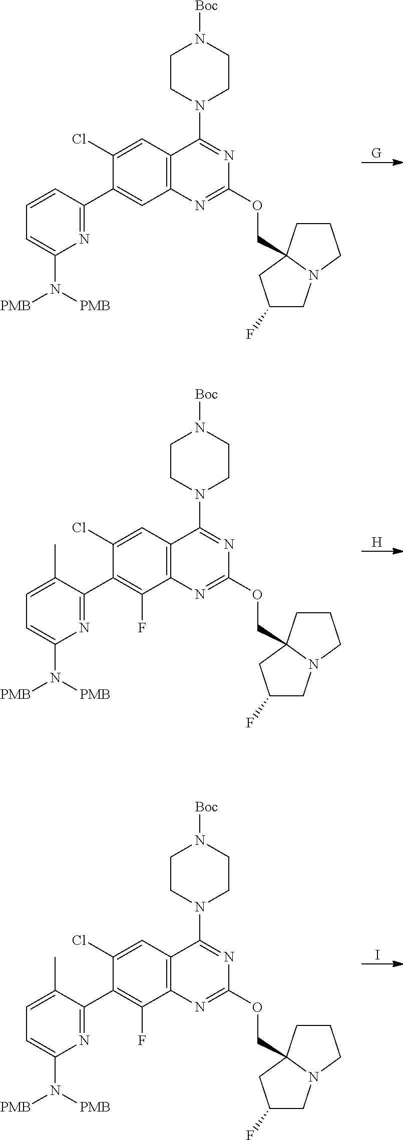

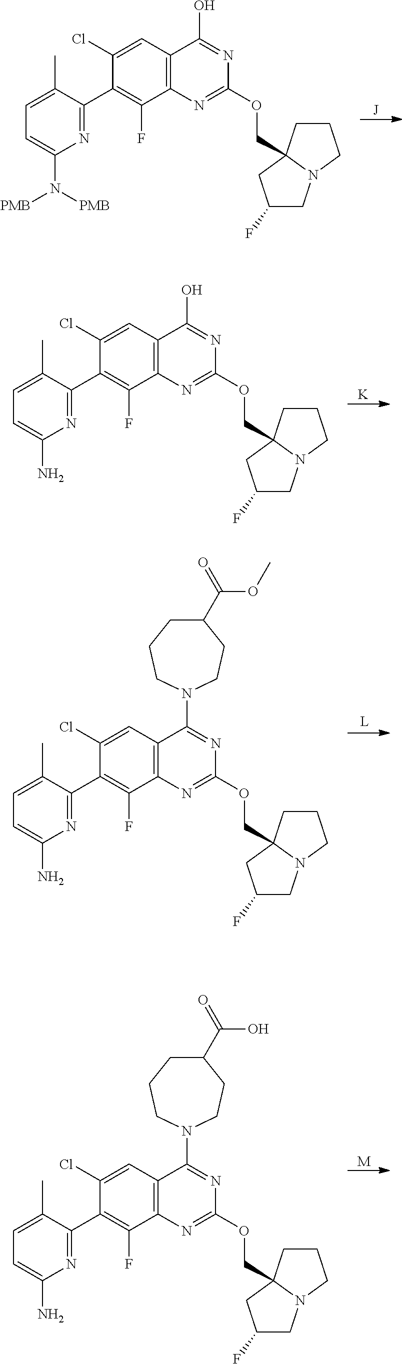

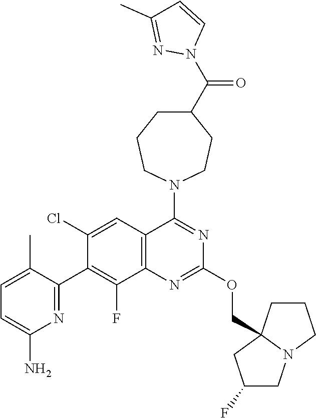 [SMILES: Cc1ccn(C(=O)C2CCCN(c3nc(OC[C@@]45CCCN4C[C@H](F)C5)nc4c(F)c(-c5nc(N)ccc5C)c(Cl)cc34)CC2)n1]

Step A. 6-bromo-N,N-bis(4-methoxybenzyl)pyridin-2-amine: To a solution of 6-bromopyridin-2-amine (12.0 g, 1.0 equiv) in DMAc (120 mL) was added NaH (8.32 g, 60% purity, 3.0 equiv) at 0 °C. The reaction was stirred at 0 °C for 0.5 hours, 1-(chloromethyl)-4-methoxybenzene (27.2 g, 2.5 equiv) was added into above mixture dropwise at 0 °C. The reaction was stirred at 20 °C for 1 hour. The mixture was quenched with ice water (500 mL) and extracted with Ethyl acetate (2×200 mL). The combined organic layers were washed brine (100 mL), dried over anhydrous sodium sulfate, concentrated and purified by column chromatography [SiO₂, PE/Ethyl acetate=1/0 to 4/1] to afford the title compound (28.0 g, 82% yield) as yellow oil; ¹H NMR (400 MHz, CHLOROFORM-d) δ=7.21-7.15 (m, 5H), 6.89-6.83 (m, 4H), 6.72 (d, J=7.6 Hz, 1H), 6.34 (d, J=8.4 Hz, 1H), 4.67 (s, 4H), 3.81 (s, 6H); LCMS (ESI, M+1, M+3): m/z=413.1, 415.1.

Step B. N,N-bis(4-methoxybenzyl)-6-(tributylstannyl)pyridin-2-amine: To a solution of 6-bromo-N,N-bis(4-methoxybenzyl)pyridin-2-amine (12.0 g, 1.0 equiv) in THF (120 mL) was added n-BuLi (2.5 M, 12.78 mL, 1.1 equiv) at −70 °C. The reaction was stirred at −70 °C for 0.5 hours. Tributylchlorostannane (15.5 g, 1.6 equiv) was added into above mixture dropwise at −60 °C. The reaction was stirred at −60 °C for 1 hour. The mixture was quenched with saturated ammonium chloride solution (200 mL) and extracted with Ethyl acetate (2×100 mL). The combined organic layers were washed with brine (2×100 mL), dried over anhydrous sodium sulfate, concentrated and purified by column chromatography [Al₂O₃, PE/Ethyl acetate=1/0 to 20/1] to afford the title compound (13.2 g, 68% yield) as yellow oil; ¹H NMR (400 MHz, CHLOROFORM-d) δ=7.25-7.15 (m, 5H), 6.88-6.79 (m, 4H), 6.72 (dd, J=0.8, 6.8 Hz, 1H), 6.30 (dd, J=0.8, 8.4 Hz, 1H), 4.71 (s, 4H), 3.80 (s, 6H), 1.65-1.50 (m, 6H), 1.39-1.26 (m, 6H), 1.15-0.96 (m, 6H), 0.87 (t, J=7.2 Hz, 9H); LCMS (ESI, M+1): m/z=625.3.

Step C. tert-butyl 4-(7-bromo-2,6-dichloro-8-fluoroquinazolin-4-yl)piperazine-1-carboxylate: To a solution of 7-bromo-2,4,6-trichloro-8-fluoroquinazoline (27.0 g, 1.0 equiv) and DIEA (31.7 g, 3.0 equiv) in DCM (270 mL) was added tert-butyl piperazine-1-carboxylate (14.5 g, 0.95 equiv) at −40 °C. The reaction was stirred at −40 °C for 0.5 hours. The mixture was diluted with water (300 mL) and extracted with DCM (2×150 mL). The combined organic layers were concentrated and triturated with ACN (150 mL) at 20 °C for 15 minutes to afford the title compound (32.0 g, 81% yield) as yellow solid; ¹H NMR (400 MHz, CHLOROFORM-d) δ=7.76 (d, J=2.0 Hz, 1H), 3.99-3.83 (m, 4H), 3.74-3.56 (m, 4H), 1.50 (s, 9H); LCMS (ESI, M+1, M+3): m/z=479.1, 481.1.

Step D. tert-butyl 4-(7-bromo-6-chloro-2,8-difluoroquinazolin-4-yl)piperazine-1-carboxylate: To a solution of tert-butyl 4-(7-bromo-2,6-dichloro-8-fluoroquinazolin-4-yl)piperazine-1-carboxylate (33.0 g, 1.0 equiv) and 18-Crown-6 (1.82 g, 0.1 equiv) in DMSO (180 mL) was added KF (39.9 g, 10 equiv). The reaction was stirred at 130 °C for 1 hour. The mixture was diluted with water (600 mL) and extracted with ethyl acetate (2×200 mL). The combined organic layers were washed with brine (3×300 mL), concentrated and triturated with ACN (100 mL) at 20 °C for 15 minutes to afford the title compound (22.5 g, 64% yield) as yellow solid; LCMS (ESI, M+1, M+3): m/z=463.1, 465.1.

Step E. tert-butyl 4-(7-bromo-6-chloro-8-fluoro-2-(((2R,7aS)-2-fluorotetrahydro-1H-pyrrolizin-7a(5H)-yl)methoxy)quinazolin-4-yl)piperazine-1-carboxylate: To a solution of tert-butyl 4-(7-bromo-6-chloro-2,8-difluoroquinazolin-4-yl)piperazine-1-carboxylate (20.5 g, 1.0 equiv) and ((2R,7aS)-2-fluorotetrahydro-1H-pyrrolizin-7a(5H)-yl)methanol (7.04 g, 1.0 equiv) in THF (200 mL) was added t-BuONa (5.10 g, 1.2 equiv) at 0 °C. The reaction was stirred at 0 °C for 0.5 hours. The mixture was diluted with water (100 mL) and extracted with ethyl acetate (2×200 mL). The combined organic layers were washed with brine (100 mL), dried over anhydrous sodium sulfate and concentrated to afford the title compound (27.5 g, 89% yield) as white solid; ¹H NMR (400 MHz, CHLOROFORM-d) δ=7.69 (d, J=2.0 Hz, 1H), 5.34-5.20 (m, 1H), 4.28-4.21 (m, 1H), 4.14 (d, J=10.8 Hz, 1H), 3.82-3.54 (m, 8H), 3.28-3.13 (m, 3H), 2.97 (dt, J=5.6, 9.2 Hz, 1H), 2.30-2.09 (m, 3H), 2.00-1.81 (m, 3H), 1.50 (s, 9H); LCMS (ESI, M+1, M+3): m/z=602.1, 604.1.

Step F. tert-butyl 4-(7-(6-(bis(4-methoxybenzyl)amino)pyridin-2-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate: To a solution of tert-butyl 4-(7-bromo-6-chloro-8-fluoro-2-(((2R,7aS)-2-fluorotetrahydro-1H-pyrrolizin-7a(5H)-yl)methoxy)quinazolin-4-yl)piperazine-1-carboxylate (10.0 g, 1.0 equiv) and N,N-bis(4-methoxybenzyl)-6-(tributylstannyl)pyridin-2-amine (11.9 g, 1.15 equiv) in DMAC (90 mL) was added CataCXium A Pd G3 (1.21 g, 0.1 equiv). The reaction was degassed and purged with nitrogen 3 times. The reaction was stirred at 90 °C for 3 hours. The mixture was diluted with water (300 mL) and extracted with Ethyl acetate (2×200 mL). The combined organic layers were washed with brine (200 mL), dried over anhydrous sodium sulfate and concentrated to afford the title compound (13.5 g, 69% yield) as yellow solid; LCMS (ESI, M+1): m/z=856.4.

Step G. tert-butyl 4-(7-(6-(bis(4-methoxybenzyl)amino)-3-iodopyridin-2-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate: To a solution of tert-butyl 4-(7-(6-(bis(4-methoxybenzyl)amino)pyridin-2-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate (6.70 g, 1.0 equiv) in AcOH (40 mL) was added NIS (1.94 g, 1.1 equiv). The reaction was stirred at 50 °C for 15 minutes. The mixture was concentrated and purified by reversed phase flash chromatography [0.1% FA condition] to afford the title compound (3.70 g, 48% yield) as white solid; ¹H NMR (400 MHz, CHLOROFORM-d) δ=7.77 (d, J=8.8 Hz, 1H), 7.70 (d, J=1.2 Hz, 1H), 7.15 (d, J=8.8 Hz, 4H), 6.84 (d, J=8.8 Hz, 4H), 6.31 (d, J=8.8 Hz, 1H), 5.39-5.16 (m, 1H), 4.76-4.66 (m, 2H), 4.61-4.50 (m, 2H), 4.28 (dd, J=6.4, 10.4 Hz, 1H), 4.20-4.15 (m, 1H), 3.80 (s, 10H), 3.66 (br d, J=4.4 Hz, 4H), 3.32-3.13 (m, 3H), 2.97 (dt, J=5.2, 8.8 Hz, 1H), 2.31-2.12 (m, 3H), 1.99-1.83 (m, 3H), 1.51 (s, 9H); LCMS (ESI, M+1): m/z=982.3.

Step H. tert-butyl 4-(7-(6-(bis(4-methoxybenzyl)amino)-3-methylpyridin-2-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate: To a mixture of tert-butyl 4-(7-(6-(bis(4-methoxybenzyl)amino)-3-iodopyridin-2-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate (3.60 g, 1.0 equiv) and 2,4,6-trimethyl-1,3,5,2,4,6-trioxatriborinane (3.5 M, 3.1 mL, 3.0 equiv) in dioxane (40 mL) were added K₂CO₃ (2 M in water, 5.5 mL, 3.0 equiv) and Pd(dppf)Cl2 (268 mg, 0.1 equiv). The reaction was degassed and purged with nitrogen 3 times. The reaction was stirred at 90 °C for 1 hour. The mixture was diluted with water (30 mL) and extracted with ethyl acetate (2×30 mL). The combined organic layers were washed with brine (50 mL), dried over anhydrous sodium sulfate and concentrated to afford the title compound (4.20 g, crude) as yellow solid; LCMS (ESI, M+1): m/z=870.4.

Step I. 7-(6-(bis(4-methoxybenzyl)amino)-3-methylpyridin-2-yl)-6-chloro-8-fluoro-2-(((2R,7aS)-2-fluorotetrahydro-1H-pyrrolizin-7a(5H)-yl)methoxy)quinazolin-4-ol: To a solution of tert-butyl 4-(7-(6-(bis(4-methoxybenzyl)amino)-3-methylpyridin-2-yl)-6-chloro-8-fluoro-2-(((2R,7aS)-2-fluorotetrahydro-1H-pyrrolizin-7a(5H)-yl)methoxy)quinazolin-4-yl)piperazine-1-carboxylate (2.70 g, 1.0 equiv) in EtOH (60 mL) was added NaOH (2 M in water, 12.4 mL, 8.0 equiv). The reaction was stirred at 50 °C for 12 hours. The mixture was diluted with water (30 mL). The mixture was adjusted to pH=8 with 1 M HCl, concentrated and extracted with ethyl acetate (3×80 mL). The combined organic layers were washed brine (30 mL), dried over anhydrous sodium sulfate and concentrated to afford the title compound (2.00 g, 71% yield) as white solid; LCMS (ESI, M+1): m/z=702.4.

Step J. 7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-ol: A solution of 7-(6-(bis(4-methoxybenzyl)amino)-3-methylpyridin-2-yl)-6-chloro-8-fluoro-2-(((2R,7aS)-2-fluorotetrahydro-1H-pyrrolizin-7a(5H)-yl)methoxy)quinazolin-4-ol (2.00 g, 1.0 equiv) in TFA (16.2 g, 50 equiv) was stirred at 50 °C for 1 hour. The mixture was concentrated and purified by reversed phase flash chromatography [0.1% FA condition] to afford the title compound (720 mg, 54% yield) as white solid; LCMS (ESI, M+1): m/z=462.3.

Step K. methyl 1-(7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a solution of 7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-ol (270 mg, 1.0 equiv) and methyl azepane-4-carboxylate hydrogen chloride (339 mg, 3.0 equiv) in DMSO (2.5 mL) were added PyBOP (456 mg, 1.5 equiv) and TEA (177 mg, 3.0 equiv). The reaction was stirred at 25 °C for 14 hours. The mixture was filtered and purified with reversed phase flash chromatography [water (0.1% formic acid)/acetonitrile=1/3] to afford the title compound (180 mg, 51% yield) as light yellow solid; LCMS (ESI, M+1): m/z=601.4.

Step L. 1-(7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl 1-(7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-yl)azepane-4-carboxylate (150 mg, 1.0 equiv) in MeOH (1.5 mL) was added LiOH·H₂O (1.5 M in H₂O, 499 μL, 3.0 equiv). The reaction was stirred at 20 °C for 1 hour. The mixture was adjusted to pH=7 with HCl (1 M), concentrated and purified with reversed phase flash [water (0.1% formic acid)/acetonitrile] to afford the title compound (131 mg, 90% yield) as light yellow solid; LCMS (ESI, M+1): m/z=587.3.

Step M. (1-(7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-yl) azepan-4-yl)(3-methyl-1H-pyrazol-1-yl)methanone: To a solution of 1-(7-(6-amino-3-methylpyridin-2-yl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (100.0 mg, 1.0 equiv) and 3-methyl-1H-pyrazole (35.0 mg, 2.5 equiv) in DMAc (1 mL) were added HATU (97.1 mg, 1.5 equiv) and TEA (51.7 mg, 3.0 equiv). The reaction was stirred at 20 °C for 10 hours. The mixture was purified with prep-HPLC [column: Waters xbridge 150×25 mm×10 μm; mobile phase: water (0.1% NH₄HCO₃)-acetonitrile; gradient: 56%-76% B over 8 minutes] and prep-HPLC [column: Waters xbridge 150×25 mm×10 μm; mobile phase: water (ammonia hydroxide v/v)-acetonitrile; gradient: 53%-83% B over 10 minutes] to afford the title compound (33.5 mg, 28% yield) as white solid; ¹H NMR (400 MHz, DMSO-d₆) δ=8.27 (d, J=2.4 Hz, 1H), 7.97 (s, 1H), 7.32 (s, 1H), 6.52-6.43 (m, 2H), 5.88 (s, 2H), 5.36-5.16 (m, 1H), 4.24-4.12 (m, 2H), 4.12-4.06 (m, 1H), 4.06-3.97 (m, 1H), 3.97-3.71 (m, 3H), 3.14-3.02 (m, 2H), 2.99 (s, 1H), 2.85-2.77 (m, 1H), 2.36-2.18 (m, 4H), 2.17-1.94 (m, 8H), 1.88 (d, J=7.2 Hz, 3H), 1.85-1.62 (m, 5H); LCMS (ESI, M+1): m/z=651.3.

### Example 223

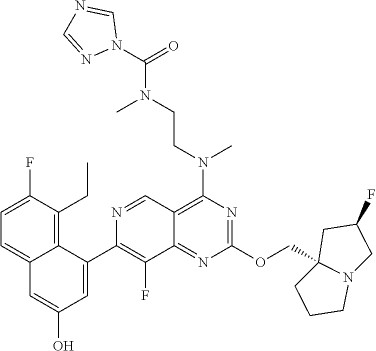 [SMILES: CCc1c(F)ccc2cc(O)cc(-c3ncc4c(N(C)CCN(C)C(=O)n5cncn5)nc(OC[C@@]56CCCN5C[C@H](F)C6)nc4c3F)c12]

### N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-1H-1,2,4-triazole-1-carboxamide

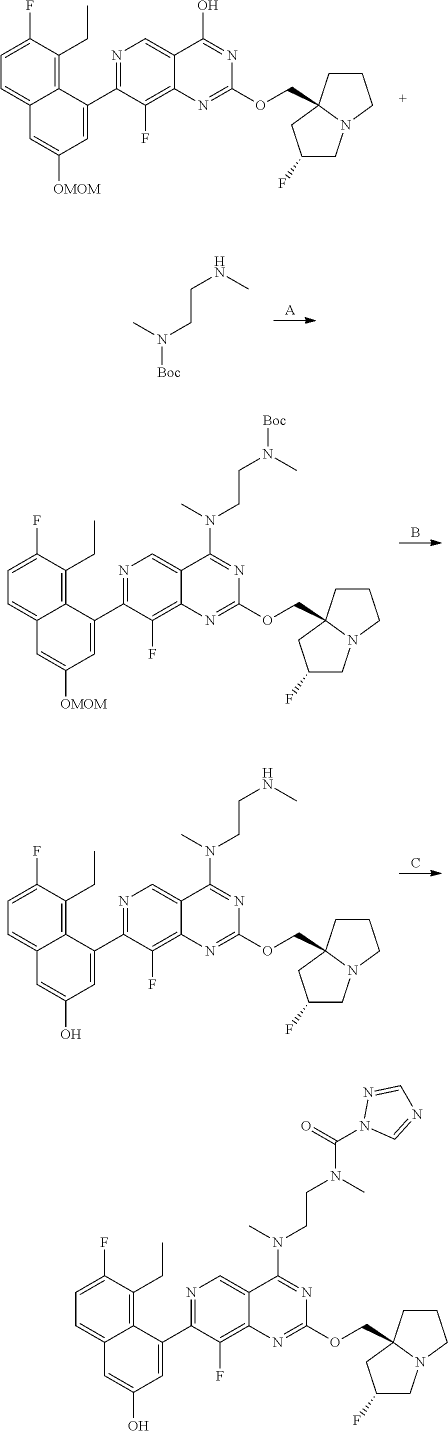

Step A. tert-butyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)(methyl)carbamate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 equiv) and diisopropylethylamine (350 mg, 3.0 equiv) in dimethylformamide (5 mL) were added tert-butyl N-methyl-N-[2-(methylamino)ethyl]carbamate (187 mg, 1.1 equiv) and benzotriazol-1-yloxy(tripyrrolidin-1-yl)phosphonium hexafluorophosphate (657 mg, 1.4 equiv) at 25 °C. The reaction was stirred at 25 °C for 12 hours. The reaction mixture was quenched by addition water (20.0 mL) at 25 °C, and then extracted with ethyl acetate (3×60.0 mL). The combined organic layers were washed with brine (3×20.0 mL), dried over anhydrous sodium sulfate, filtered, concentrated under reduced pressure and purified by column chromatography [SiO₂, petroleum ether/ethyl acetate=3/1 to dichloromethane/methyl alcohol=10/1] to afford the title compound (794 mg, 97% yield) as a yellow oil; LCMS (ESI, M+1): m/z=725.3.

Step B. 5-ethyl-6-fluoro-4-(8-fluoro-2-(((2R,7aS)-2-fluorotetrahydro-1H-pyrrolizin-7a(5)-yl)methoxy)-4-(methyl(2-(methylamino)ethyl)amino)pyrido [4,3-d]pyrimidin-7-yl)naphthalen-2-ol: To a solution of tert-butyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)(methyl)carbamate (150 mg, 1.0 equiv) in dichloromethane (3.00 mL) was added trifluoroacetic acid (1.00 mL) at 25 °C. The reaction was stirred at 25 °C for 1 hour. The mixture was concentrated under reduced pressure. The residue was diluted with methyl alcohol (20.0 mL) and adjusted to pH=8 with sodium bicarbonate solid, and then filtered. The filtrate was concentrated under reduced pressure to afford the title compound (95 mg, 99% yield) as a yellow oil; LCMS (ESI, M+1): m/z=581.3.

Step C. N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-N-methyl-1H-1,2,4-triazole-1-carboxamide: To a solution of 5-ethyl-6-fluoro-4-(8-fluoro-2-(((2R,7aS)-2-fluorotetrahydro-1H-pyrrolizin-7a(5H)-yl)methoxy)-4-(methyl(2-(methylamino)ethyl)amino)pyrido [4,3-d]pyrimidin-7-yl)naphthalen-2-ol (95 mg, 1.0 equiv) in dimethylformamide (2 mL) were added diisopropylethylamine (212 mg, 10.0 equiv) and bis(1,2,4-triazol-1-yl)methanone (32.2 mg, 1.2 equiv) at 25 °C. The reaction was stirred at 25 °C for 2 hours. The mixture was filtered to give a filtrate. The filtrate was purified by prep-HPLC [Phenomenex luna C18 15×25 mm×10 μm; A: water (0.1% FA), B: ACN; B %: 18% to 48% over 9 min] and prep-HPLC [Waters Xbridge 150×25 mm×5 μm; A: water (10 mM NH₄HCO₃), B: ACN, B %: 35% to 65%, over 9 min] to afford the title compound (13.2 mg, 12% yield) as a white solid; ¹H NMR (400 MHz, methanol-d₄) δ=9.25 (s, 1H), 8.95-8.82 (m, 1H), 8.05 (s, 1H), 7.67 (dd, J=6.0, 9.2 Hz, 1H), 7.30 (d, J=2.4 Hz, 1H), 7.25 (t, J=9.2 Hz, 1H), 7.05 (d, J=2.0 Hz, 1H), 5.42-5.18 (m, 1H), 4.39-4.23 (m, 4H), 4.20-3.96 (m, 2H), 3.71 (br s, 3H), 3.37-3.32 (m, 3H), 3.28-3.11 (m, 3H), 3.07-2.97 (m, 1H), 2.56-2.38 (m, 1H), 2.34-2.06 (m, 4H), 2.04-1.82 (m, 3H), 0.83-0.73 (m, 3H); LCMS (ESI, M+1): m/z=676.3.

### Example 224

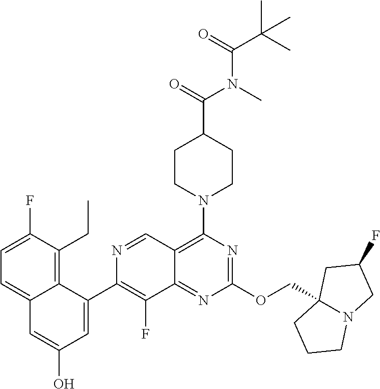 [SMILES: CCc1c(F)ccc2cc(O)cc(-c3ncc4c(N5CCC(C(=O)N(C)C(=O)C(C)(C)C)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-pivaloylpiperidine-4-carboxamide

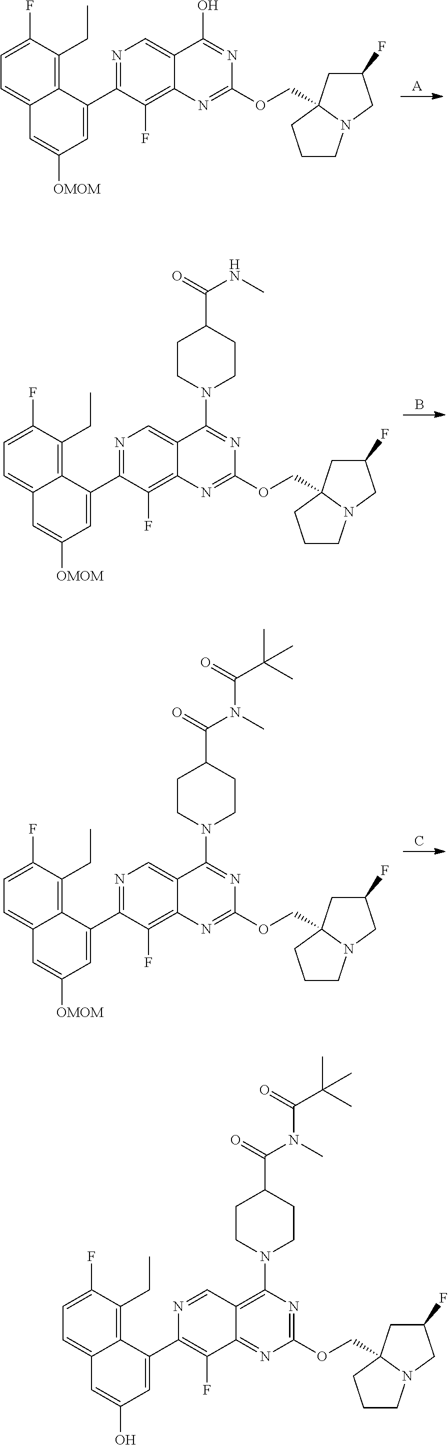

Step A. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (2 g, 1.0 equiv) and N-methylpiperidine-4-carboxamide (1.29 g, 2 equiv, HCl salt) in DMF (20 mL) were added PyBOP (2.82 g, 1.5 equiv) and DIEA (4.66 g, 10 equiv). The reaction was stirred at 25 °C for 1 hour. The mixture was filtered and the filtrate was purified by reversed phase flash chromatography [C18, 0.1% formic acid condition] to afford the title compound (1.56 g, 63% yield) as yellow solid; LCMS (ESI, M+1): m/z=679.4.

Step B. 1-(7-(8-ethyl-7-fluoro-3-(methoxymethoxy)naphthalen-1-yl)-8-fluoro-2-yl)-N-methyl-N-pivaloylpiperidine-4-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpiperidine-4-carboxamide (100 mg, 1.0 equiv) and pivaloyl chloride (75 mg, 4.2 equiv) in 2-methyltetrahydrofuran (1 mL) was added DIEA (190 mg, 10 equiv). The reaction was stirred at 80 °C for 1 hour. The mixture was diluted with water (5 mL) and extracted with ethyl acetate (3×3 mL). The organic layer was dried over sodium sulfate, filtered, concentrated and purified by reversed phase flash chromatography [C18, 0.1% formic acid condition] to afford the title compound (90 mg, 79% yield) as yellow solid; LCMS (ESI, M+1): m/z=763.4.

Step C. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-pivaloylpiperidine-4-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-pivaloylpiperidine-4-carboxamide (90 mg, 1.0 equiv) in DCM (0.5 mL) was added TFA (1.54 g, 114 equiv). The reaction was stirred at 0 °C for 0.5 hours. The mixture was concentrated, basified with saturated NaHCO₃ solution (10 mL) and extracted with ethyl acetate (3×5 mL). The combined organic layers were dried over anhydrous sodium sulfate, concentrated and purified by prep-HPLC [Waters Xbridge 150×25 mm×5 μm; A: water (NH₄HCO₃), B: ACN, B %: 52%-82% over 9 min] to afford the title compound (6.01 mg, 6.9% yield) as white solid; ¹H NMR (400 MHz, CHLOROFORM-d) δ=8.95 (s, 1H), 7.61-7.50 (m, 1H), 7.23-7.16 (m, 2H), 7.08-6.98 (m, 1H), 5.43-5.19 (m, 1H), 4.69-4.53 (m, 2H), 4.49-4.24 (m, 2H), 3.35 (br d, J=3.2 Hz, 4H), 3.20 (s, 3H), 3.15-2.93 (m, 2H), 2.18 (br d, J=6.0 Hz, 5H), 2.08-1.73 (m, 8H), 1.38 (s, 9H), 0.89-0.76 (m, 3H); LCMS (ESI, M+1): m/z=719.4.

### Example 225

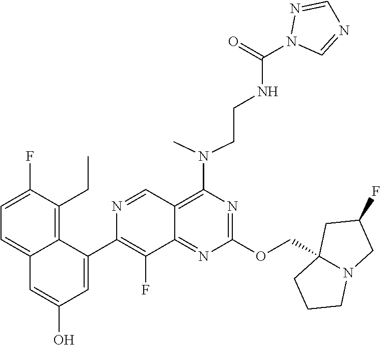 [SMILES: CCc1c(F)ccc2cc(O)cc(-c3ncc4c(N(C)CCNC(=O)n5cncn5)nc(OC[C@@]56CCCN5C[C@H](F)C6)nc4c3F)c12]

### N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-1H-1,2,4-triazole-1-carboxamide

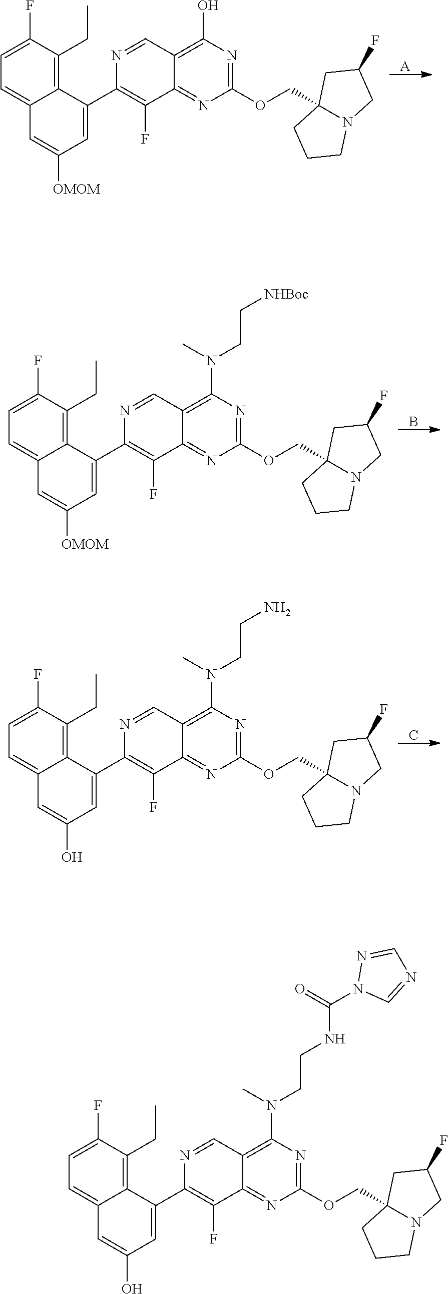

Step A. tert-butyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)carbamate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (900 mg, 1.0 equiv) in DMF (10 mL) were added PyBOP (1.01 g, 1.2 equiv) and DIEA (629 mg, 3.0 equiv). The reaction was stirred at 25 °C for 20 minutes. tert-butyl(2-(methylamino)ethyl)carbamate (848 mg, 3.0 equiv) was added into the mixture. The reaction was stirred at 25 °C for 40 minutes. The mixture was filtered and purified by reversed-phase flash (0.1% NH₃·H₂O condition) to afford the title compound (900 mg, 78% yield) as yellow solid; LCMS (ESI, M+1): m/z=711.3.

Step B. 4-(4-((2-aminoethyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: A solution of tert-butyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)carbamate (400 mg, 1.0 equiv) in HCl·MeOH (2 M, 8 mL, 28 equiv) was stirred at 25 °C for 1 hour. The mixture was concentrated, dissolved with MeOH (5 mL), adjusted to pH=7 using NaHCO₃solid, concentrated and purified by reversed-phase flash (0.1% FA condition) to afford the title compound (300 mg, 94%) as yellow solid.

Step C. N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-1H-1,2,4-triazole-1-carboxamide: To a solution of 4-(4-((2-aminoethyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol (50.0 mg, 1.0 equiv) and di(1H-1,2,4-triazol-1-yl)methanone (13.0 mg, 0.9 equiv) in dichloromethane (2 mL) was added DIEA (34.2 mg, 3.0 equiv). The reaction was stirred at 25 °C for 1 hour. The mixture was filtered, concentrated and purified by prep-HPLC [Phenomenex luna C18 250×50 mm×15 μm; A: water (FA), B %: ACN, B %: 12%-42% over 9 min] to afford the title compound (26.0 mg, 42% yield, HCOOH) as white solid; ¹H NMR (400 MHz, CHLOROFORM-d) δ=9.00-8.79 (m, 2H), 8.55 (s, 1H), 8.21-7.89 (m, 1H), 7.46 (br s, 1H), 7.19-7.09 (m, 2H), 7.09-6.84 (m, 1H), 5.39-5.18 (m, 1H), 4.53-4.27 (m, 2H), 3.90-3.67 (m, 3H), 3.66-3.39 (m, 3H), 3.35-3.20 (m, 1H), 3.18-2.96 (m, 4H), 2.56-2.28 (m, 2H), 2.27-2.11 (m, 2H), 2.10-1.88 (m, 4H), 0.70 (br s, 3H); LCMS (ESI, M+1): m/z=662.3.

### Example 226

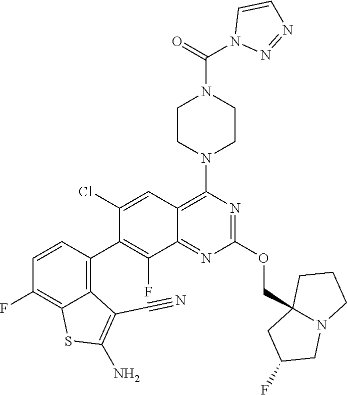 [SMILES: N#Cc1c(N)sc2c(F)ccc(-c3c(Cl)cc4c(N5CCN(C(=O)n6ccnn6)CC5)nc(OC[C@@]56CCCN5C[C@H](F)C6)nc4c3F)c12]

### 4-(4-(4-(1H-1,2,3-triazole-1-carbonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

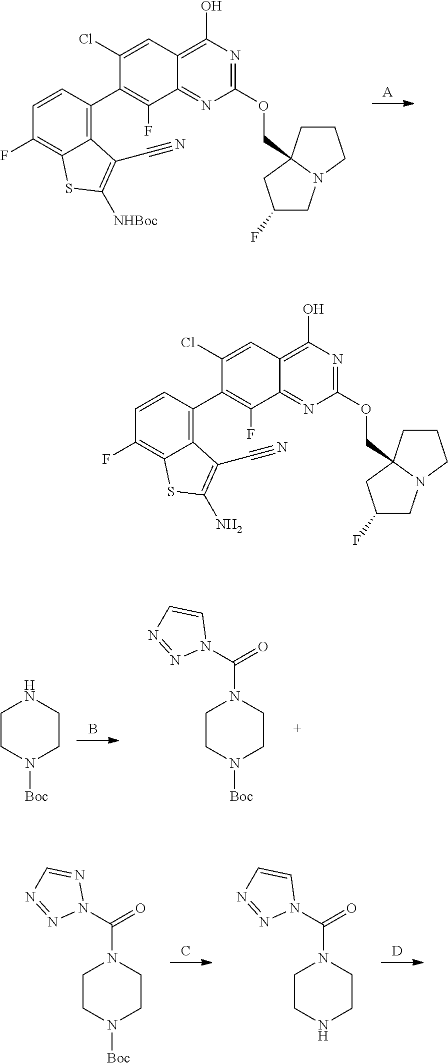

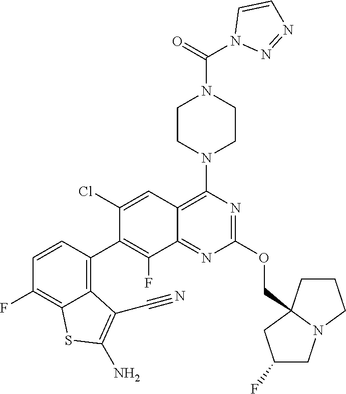 [SMILES: N#Cc1c(N)sc2c(F)ccc(-c3c(Cl)cc4c(N5CCN(C(=O)n6ccnn6)CC5)nc(OC[C@@]56CCCN5C[C@H](F)C6)nc4c3F)c12]

Step A. 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (200 mg, 1.0 equiv) in DCM (2 mL) was added TFA (2 mL, 87.0 equiv). The reaction was stirred at 25 °C for 15 minutes. The mixture was concentrated, basified with saturated NaHCO₃ solution (10 mL) and extracted with ethyl acetate (3×15 mL). The combined organic layers were dried over anhydrous sodium sulfate, concentrated to afford the title compound (133 mg, crude) as yellow solid.

Step B. tert-butyl 4-(2H-1,2,3-triazole-2-carbonyl)piperazine-1-carboxylate: To a solution of 1H-1,2,3-triazole (1.48 g, 2.0 equiv) in DCM (10 mL) were added bis(trichloromethyl) carbonate (956 mg, 0.3 equiv) and TEA (2.17 g, 2.0 equiv). The reaction was stirred at 0 °C for 1 hour. tert-butyl piperazine-1-carboxylate (2 g, 1.0 equiv) and TEA (2.17 g, 2.0 equiv) in DCM (10 mL) were added to the mixture. The reaction was stirred at 25 °C for 1 hour. The mixture was washed with HCl (2M in H₂O, 20 mL) and NaHCO₃ solution (30 mL). The organic layer was dried over anhydrous sodium sulfate, concentrated and purified with SFC separation [column: DAICEL CHIRALPAK IC, 250×30 mm, 10 μm; A: CO₂, B: MeOH (0.1% NH₃H₂O), B %: 30% B isocratic elution mode] to afford to afford two isomers.

Isomer 1: tert-butyl 4-(1H-1,2,3-triazole-1-carbonyl)piperazine-1-carboxylate (720 mg, 22% yield) as white solid; LCMS (ESI, M+1): m/z=304.1; SFC>99% ee, column: Chiralpak IC-3 50×4.6 mm I.D., 3 μm; mobile phase: 20% to 60% MeOH (0.05% DEA) in CO₂, flow rate: 3 mL/min, detector: PDA, t_{R}: 0.613 min.

Isomer 2: tert-butyl 4-(2H-1,2,3-triazole-2-carbonyl)piperazine-1-carboxylate (1.3 g, 42% yield) as white solid; LCMS (ESI, M+23): m/z=304.1; SFC=98% ee, column: Chiralpak IC-3 50×4.6 mm I.D., 3 μm; mobile phase: 20% to 60% MeOH (0.05% DEA) in CO₂, flow rate: 3 mL/min, detector: PDA, t_{R}: 0.819 min.

Step C. piperazin-1-yl(1H-1,2,3-triazol-1-yl)methanone: To a solid of tert-butyl 4-(1H-1,2,3-triazole-1-carbonyl)piperazine-1-carboxylate (100 mg, 1.0 equiv) in MeOH (0.2 mL) was added HCl·MeOH (2 M, 1.0 mL). The reaction was stirred at 25 °C for 0.5 hours. The mixture was concentrated to afford the title compound (70 mg, crude, HCl salt) as white solid.

Step D. 4-(4-(4-(1H-1,2,3-triazole-1-carbonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (100 mg, 1.0 equiv) and piperazin-1-yl(1H-1,2,3-triazol-1-yl)methanone (70 mg, 1.76 equiv, HCl salt) in DMF (1 mL) were added PyBOP (143 mg, 1.5 equiv) and DIEA (237 mg, 10 equiv). The reaction was stirred at 25 °C for 0.5 hours. The mixture was filtered and the filtrate was purified by reversed phase flash chromatography [C18, 0.1% formic acid condition] and prep-HPLC [Waters Xbridge 150×25 mm×5 μm; A: water (NH₄HCO₃), B: ACN, B %: 42%-72% over 9 min] to afford the title compound (12.4 mg, 9.5% yield) as white solid; ¹H NMR (400 MHz, CHLOROFORM-d) δ=8.24 (d, J=1.2 Hz, 1H), 7.77 (dd, J=1.2, 6.1 Hz, 2H), 7.23 (dd, J=5.2, 8.4 Hz, 1H), 7.05 (t, J=8.8 Hz, 1H), 5.46 (br s, 2H), 5.40-5.20 (m, 1H), 4.47-3.83 (m, 10H), 3.41-3.16 (m, 3H), 3.07-2.95 (m, 1H), 2.37-2.11 (m, 3H), 2.05-1.86 (m, 3H); LCMS (ESI, M+1): m/z=709.2.

### Example 227

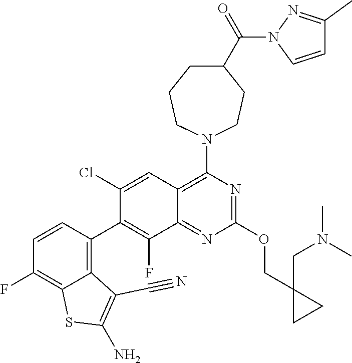 [SMILES: Cc1ccn(C(=O)C2CCCN(c3nc(OCC4(CN(C)C)CC4)nc4c(F)c(-c5ccc(F)c6sc(N)c(C#N)c56)c(Cl)cc34)CC2)n1]

### 2-amino-4-(6-chloro-2-((1-((dimethylamino)methyl)cyclopropyl)methoxy)-8-fluoro-4-(4-(3-methyl-1H-pyrazole-1-carbonyl) azepan-1-yl)quinazolin-7-yl)-7-fluorobenzo[b]thiophene-3-carbonitrile

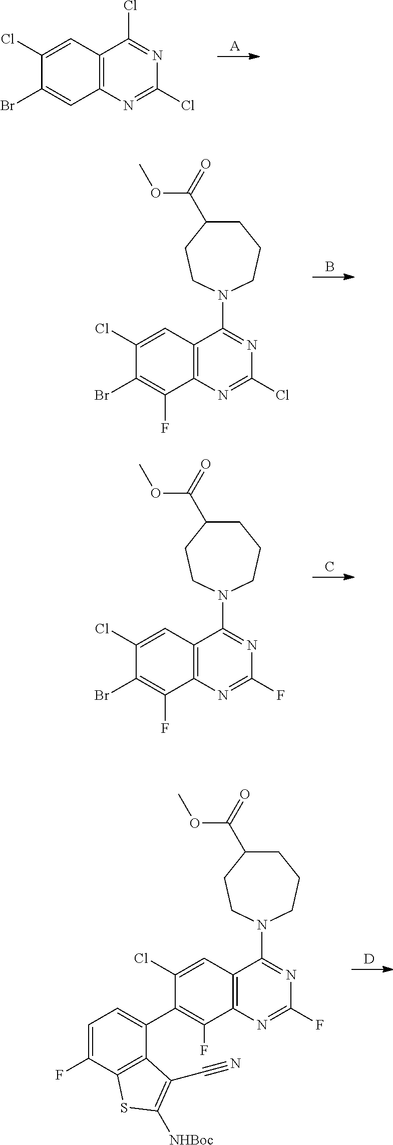

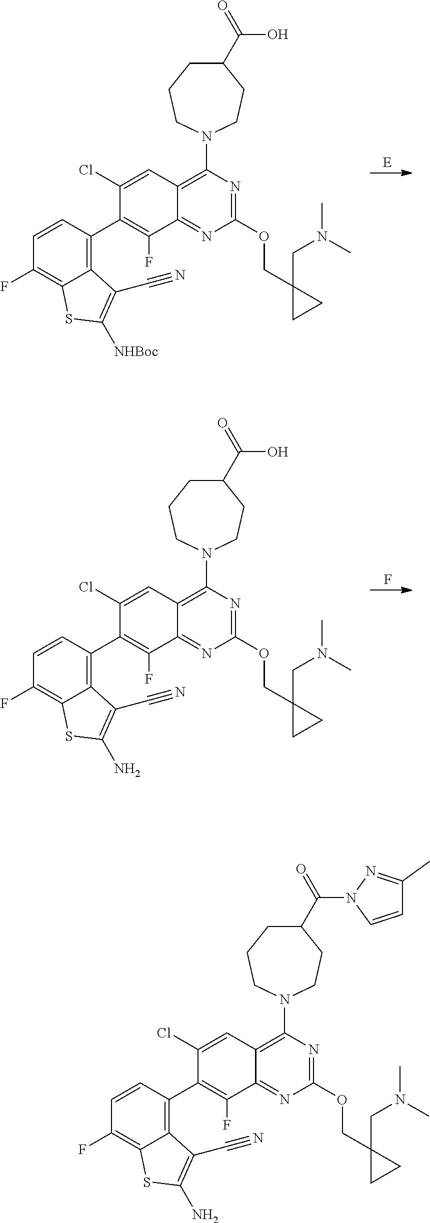

Step A. methyl 1-(7-bromo-2,6-dichloro-8-fluoroquinazolin-4-yl)azepane-4-carboxylate: To a solution of 7-bromo-2,4,6-trichloro-8-fluoroquinazoline (1 g, 1.0 equiv) in DCM (10 mL) were added DIEA (1.17 g, 3.0 equiv) and methyl azepane-4-carboxylate (428 g, 0.9 equiv). The reaction was stirred at −40 °C for 30 mins. The mixture was diluted with water (20 mL) and extracted with Ethyl acetate (2×50 mL). The combined organic layers were washed with brine (2×5 mL), dried over anhydrous sodium sulfate, concentrated and triturated with ethyl acetate (5 mL) to afford the title compound (900 mg, 62% yield) as white solid; LCMS (ESI, M+1, M+3): m/z=450.1, 452.1.

Step B. methyl 1-(7-bromo-6-chloro-2,8-difluoroquinazolin-4-yl)azepane-4-carboxylate: To a solution of methyl 1-(7-bromo-2,6-dichloro-8-fluoroquinazolin-4-yl)azepane-4-carboxylate (900 mg, 1 equiv) in DMSO (9 mL) were added 18-CROWN-6 (52.7 mg, 0.1 equiv) and KF (2.32 g, 20 equiv). The reaction was stirred at 120 °C for 3 hours. The mixture was purified by reversed phase flash chromatography [C18, 0.1% formic acid condition] to afford the title compound (850 mg, 91% yield) as yellow solid; LCMS (ESI,M+1, M+3): m/z=434.0, 436.0.

Step C Methyl 1-(7-(2-((tert-butoxycarbonyl)amino)-3-cyano-7-fluorobenzo[b]thiophen-4-yl)-6-chloro-2,8-difluoroquinazolin-4-yl)azepane-4-carboxylate: To a solution of methyl 1-(7-bromo-6-chloro-2,8-difluoroquinazolin-4-yl)azepane-4-carboxylate (740 mg, 1.0 equiv) and tert-butyl(3-cyano-4-(5,5-dimethyl-1,3,2-dioxaborinan-2-yl)-7-fluorobenzo[b]thiophen-2-yl)carbamate (1.38 g, 2.0 equiv) in THF (7.5 mL) were added Cs₂CO₃ (1.11 g, 2.0 equiv) and Pd(DPEphos)Cl₂ (122 mg, 0.1 equiv). The reaction was degassed and purged with nitrogen 3 times. The reaction was stirred at 60 °C for 4 hours. The mixture was diluted with water (30 mL), extracted with Ethyl acetate (2×50 mL). The combined organic layers were washed with brine (2×5 mL), dried over anhydrous sodium sulfate, concentrated and purified by reversed phase flash chromatography [C18, 0.1% formic acid condition] to afford the title compound (250 mg, 19% yield) as yellow solid; LCMS (ESI, M+1): m/z=646.1.

Step D. 1-(7-(2-((tert-butoxycarbonyl)amino)-3-cyano-7-fluorobenzo[b]thiophen-4-yl)-6-chloro-2-((1-((dimethylamino)methyl)cyclopropyl)methoxy)-8-fluoroquinazolin-4-yl)azepane-4-carboxylic acid: To a solution of [1-[(dimethylamino)methyl]cyclopropyl]methanol (21.6 mg, 1.0 equiv) in THF (2 mL) were added t-BuONa (48.1 mg, 3.0 equiv). The reaction was stirred at 0 °C for 15 mins. And then the mixture was added methyl 1-(7-(2-((tert-butoxycarbonyl)amino)-3-cyano-7-fluorobenzo[b]thiophen-4-yl)-6-chloro-2,8-difluoroquinazolin-4-yl)azepane-4-carboxylate (130 mg, 1.0 equiv) and stirred at 0 °C for 45 mins. The mixture was diluted with water (10 mL), extracted with ethyl acetate (2×20 mL). The combined organic layers were washed with brine (2×5 mL), dried over anhydrous sodium sulfate, concentrated and purified by reversed phase flash chromatography [C18, 0.1% formic acid condition] to afford the title compound (40 mg, 32% yield) as white solid; LCMS (ESI, M+1): m/z=741.3.

Step E. 1-(7-(2-amino-3-cyano-7-fluorobenzo[b]thiophen-4-yl)-6-chloro-2-((1-((dimethylamino)methyl)cyclopropyl)methoxy)-8-fluoroquinazolin-4-yl)azepane-4-carboxylic acid: To a solution of 1-(7-(2-((tert-butoxycarbonyl)amino)-3-cyano-7-fluorobenzo[b]thiophen-4-yl)-6-chloro-2-((1-((dimethylamino)methyl)cyclopropyl)methoxy)-8-fluoroquinazolin-4-yl)azepane-4-carboxylic acid (30 mg, 1.0 equiv) in DCM (0.3 mL) were added TFA (460 mg, 99.79 equiv). The reaction was stirred at 25 °C for 10 minutes. The mixture was filtered and concentrated to afford the title compound (25 mg, crude) as red oil; LCMS (ESI, M+1): m/z=641.3.

Step F. 2-amino-4-(6-chloro-2-((1-((dimethylamino)methyl)cyclopropyl)methoxy)-8-fluoro-4-(4-(3-methyl-1H-pyrazole-1-carbonyl) azepan-1-yl)quinazolin-7-yl)-7-fluorobenzo[b]thiophene-3-carbonitrile: To a mixture of 1-(7-(2-amino-3-cyano-7-fluorobenzo[b]thiophen-4-yl)-6-chloro-2-((1-((dimethylamino)methyl)cyclopropyl)methoxy)-8-fluoroquinazolin-4-yl)azepane-4-carboxylic acid (25 mg, 1.0 equiv) and 5-methyl-1H-pyrazole (32 mg, 10.0 equiv) in DMF (0.5 mL) were added HATU (44 mg, 3.0 equiv) and DIEA (100 mg, 20.0 equiv). The reaction was stirred at 30 °C for 10 minutes. The mixture was filtered and purified by reversed phase flash [water/ACN] to afford the title compound (8.65 mg, 30% yield) as off-white solid; ¹H NMR (400 MHz, CHLOROFORM-d) δ=8.17 (t, J=2.0 Hz, 1H), 7.91 (br d, J=6.0 Hz, 1H), 7.26-7.20 (m, 1H), 7.08-7.01 (m, 1H), 6.28 (d, J=2.8 Hz, 1H), 5.61-5.34 (m, 2H), 4.36-4.19 (m, 3H), 4.00-3.82 (m, 3H), 2.50-2.38 (m, 4H), 2.35 (d, J=1.2 Hz, 4H), 2.31-2.19 (m, 4H), 2.17-2.09 (m, 1H), 2.05-1.83 (m, 2H), 1.82-1.66 (m, 3H), 0.91-0.72 (m, 4H); LCMS (ESI, M+1): m/z=705.3.

### Example 228

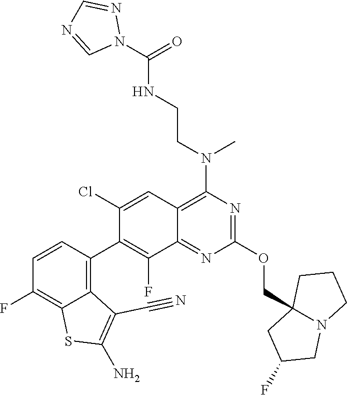 [SMILES: CN(CCNC(=O)n1cncn1)c1nc(OC[C@@]23CCCN2C[C@H](F)C3)nc2c(F)c(-c3ccc(F)c4sc(N)c(C#N)c34)c(Cl)cc12]

### N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-1H-1,2,4-triazole-1-carboxamide

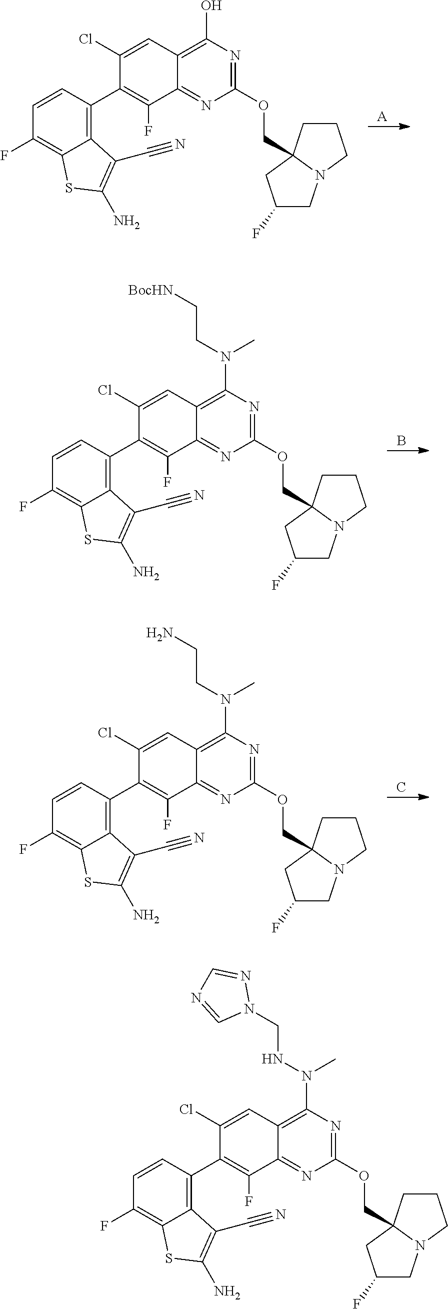

Step A. tert-butyl(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)carbamate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (150 mg, 1.0 equiv) in DMF (2 mL) were added PyBOP (172 mg, 1.2 equiv) and DIEA (107 mg, 3.0 equiv). The reaction was stirred at 25 °C for 20 minutes. tert-butyl(2-(methylamino)ethyl)carbamate (144 mg, 3.0 equiv) was added into the mixture. The reaction was stirred at 25 °C for 40 minutes. The mixture was filtered and purified by prep-HPLC [Waters Xbridge Prep OBD C18 150×40 mm×10 μm; A: water (NH₄HCO₃), B %: ACN, B %: 48%-78% over 20 min] to afford the title compound (110 mg, 55% yield) as white solid; LCMS (ESI, M+1): m/z=702.2.

Step B. 2-amino-4-(4-((2-aminoethyl)(methyl)amino)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)carbamate (110 mg, 1.0 equiv) in dichloromethane (2 mL) was added TFA (1.54 g, 86 equiv) at 0 °C. The reaction was stirred at 25 °C for 1 hour. The mixture was concentrated, dissolved with MeOH (5 mL), adjusted to pH=7 by using NaHCO₃solid, concentrated and purified by reversed-phase flash (0.1% FA condition) to afford the title compound (70.0 mg, 68% yield, HCOOH) as white solid; LCMS (ESI, M+1): m/z=602.2.

Step C. N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-1H-1,2,4-triazole-1-carboxamide: To a solution of 2-amino-4-(4-((2-aminoethyl)(methyl)amino)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (30.0 mg, 1.0 equiv, HCOOH) and di(1H-1,2,4-triazol-1-yl)methanone (9.12 mg, 1.2 equiv) in dichloromethane (1 mL) was added DIEA (18.0 mg, 3.0 equiv). The reaction was stirred at 25 °C for 1 hour. The mixture was filtered, concentrated and purified by prep-HPLC [Waters Xbridge 150×25 mm×5 μm; A: water (NH₄HCO₃), B %: ACN, B %: 38%-68% over 9 min] to afford the title compound (9.95 mg, 30% yield) as white solid; ¹H NMR (400 MHz, CHLOROFORM-d) δ=8.85 (s, 1H), 8.26-8.13 (m, 1H), 7.69 (br s, 1H), 7.18 (dd, J=5.2, 8.4 Hz, 1H), 7.01 (t, J=8.8 Hz, 1H), 5.74 (br s, 2H), 5.42-5.16 (m, 1H), 4.34-4.24 (m, 1H), 4.23-4.11 (m, 1H), 4.04-3.78 (m, 4H), 3.34-3.13 (m, 6H), 3.04-2.91 (m, 1H), 2.33-2.25 (m, 1H), 2.23-2.11 (m, 2H), 2.02-1.88 (m, 3H); LCMS (ESI, M+1): m/z=697.2.

### Example 229

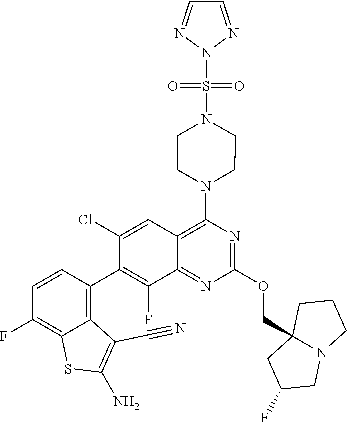 [SMILES: N#Cc1c(N)sc2c(F)ccc(-c3c(Cl)cc4c(N5CCN(S(=O)(=O)n6nccn6)CC5)nc(OC[C@@]56CCCN5C[C@H](F)C6)nc4c3F)c12]

### 4-(4-(4-((2H-1,2,3-triazol-2-yl)sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

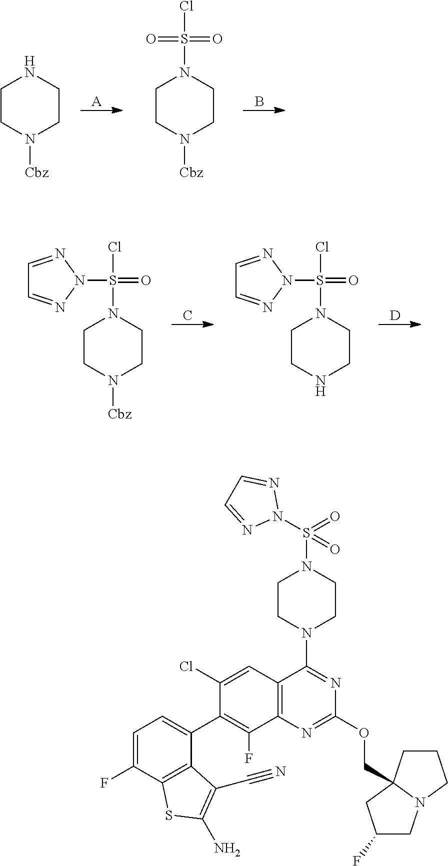

Step A. benzyl 4-(chlorosulfonyl)piperazine-1-carboxylate: To a solution of sulfuryl dichloride (1.46 g, 2.5 equiv) in acetonitrile (20.0 mL) was added benzyl piperazine-1-carboxylate (950 mg, 1.0 equiv) at 0 °C, then triethylamine (1.16 g, 2.7 equiv) was added dropwise at 0 °C and stirred for 0.5 hour. The reaction was stirred at 50 °C for 2 hours. The mixture was quenched with saturated bicarbonate aqueous solution (50.0 mL) and extracted with ethyl acetate (2×30 mL). The combined organic layers were washed with brine (2×20 mL), dried over anhydrous sodium sulfate, concentrated to afford the title compound (1.35 g, 98% yield) as a yellow oil; ¹H NMR (400 MHz, chloroform-d) δ=7.45-7.32 (m, 5H), 5.17 (s, 2H), 3.73 (br s, 4H), 3.47-3.01 (m, 4H).

Step B. benzyl 4-((2H-1,2,3-triazol-2-yl)sulfonyl)piperazine-1-carboxylate: To a solution of 2H-1,2,3-triazole (400 mg, 1.0 equiv) and benzyl 4-(chlorosulfonyl)piperazine-1-carboxylate (1.80 g, 1.0 equiv) in ethyl alcohol (10.0 mL) was added potassium carbonate (660 mg, 1.0 equiv) at 25 °C. The reaction was stirred at 25 °C for 12 hours. The mixture was directly concentrated and purified by prep-HPLC [column: Phenomenex luna C18 150×40 mm×15 μm; A: water (0.1% FA), B: ACN; B %: 38%-68% over 15 min] to afford the title compound (400 mg, 19% yield) as yellow solid; ¹H NMR (400 MHz, chloroform-d) δ=7.87 (s, 2H), 7.40-7.31 (m, 5H), 5.12 (s, 2H), 3.66-3.55 (m, 4H), 3.43 (br d, J=4.0 Hz, 4H).

Step C. 1-((2H-1,2,3-triazol-2-yl)sulfonyl)piperazine; To a solution of benzyl 4-((2H-1,2,3-triazol-2-yl)sulfonyl)piperazine-1-carboxylate (400 mg, 1.0 equiv) in ethyl alcohol (10.0 mL) was added Pd(OH)₂ (775 mg, 10% w/w) under a nitrogen atmosphere at 25 °C. The suspension was degassed under vacuum and purged with hydrogen for several times. The reaction was stirred under hydrogen (15 psi) at 25 °C for 1 hour. The mixture was filtered and the filter was concentrated to afford the title compound (180 mg, 68% yield) as a yellow oil which was used directly; ¹H NMR (400 MHz, chloroform-d) δ=7.90-7.84 (m, 2H), 3.44-3.35 (m, 4H), 2.97-2.87 (m, 4H).

Step D. 4-(4-(4-((2H-1,2,3-triazol-2-yl)sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (100 mg, 1.0 equiv) and 1-((2H-1,2,3-triazol-2-yl)sulfonyl)piperazine (150 mg, 3.4 equiv) in dimethylformamide (0.50 mL) were added diisopropylethylamine (71.0 mg, 3.0 equiv) and benzotriazol-1-yloxy(tripyrrolidin-1-yl)phosphonium hexafluorophosphate (114 mg, 1.2 equiv) at 25 °C. The reaction was stirred at 25 °C for 2 hours. The mixture was purified by prep-HPLC [Waters Xbridge 150×25 mm×5 μm; A: water (10 mM NH₄HCO₃), B: ACN, B %: 50%-80% over 9 min] and prep-HPLC [Phenomenex luna C18 150×25 mm×10 μm; A: water (0.1% FA), B: ACN; B %: 20%-50% over 9 min] to afford the title compound (6.06 mg, 4.4% yield) as an off-white solid; ¹H NMR (400 MHz, methanol-d₄) δ=8.03 (s, 2H), 7.89 (s, 1H), 7.20 (dd, J=5.2, 8.4 Hz, 1H), 7.07-7.01 (m, 1H), 5.54-5.36 (m, 1H), 4.60-4.40 (m, 2H), 4.08-3.96 (m, 4H), 3.68 (br t, J=5.2 Hz, 4H), 3.64-3.55 (m, 2H), 3.25 (br d, J=2.8 Hz, 2H), 2.60-2.36 (m, 2H), 2.33-2.14 (m, 3H), 2.10-1.95 (m, 1H); LCMS (ESI, M+1): m/z=745.2.

### Example 230

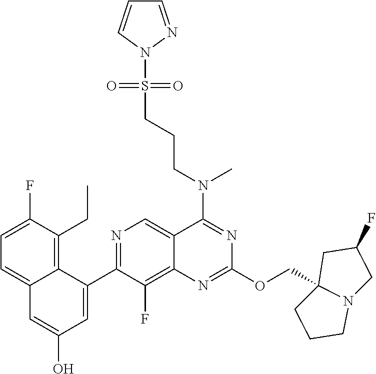 [SMILES: CCc1c(F)ccc2cc(O)cc(-c3ncc4c(N(C)CCCS(=O)(=O)n5cccn5)nc(OC[C@@]56CCCN5C[C@H](F)C6)nc4c3F)c12]

### 4-(4-((3-((1H-pyrazol-1-yl)sulfonyl)propyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

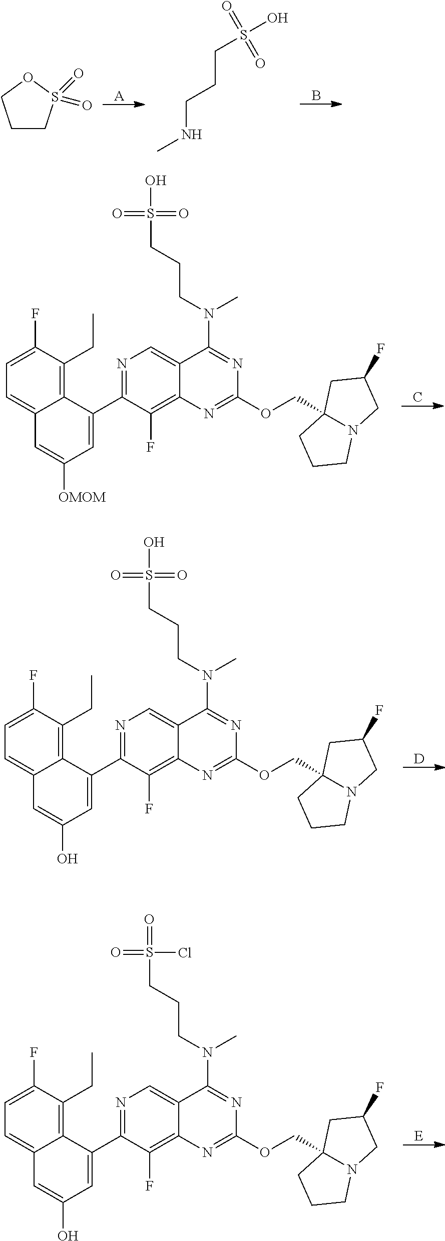

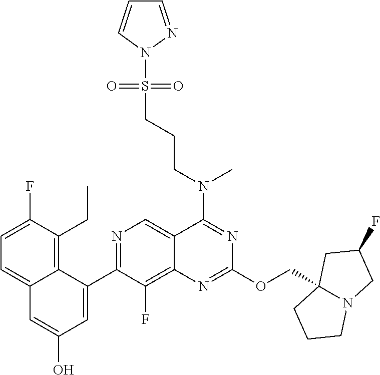 [SMILES: CCc1c(F)ccc2cc(O)cc(-c3ncc4c(N(C)CCCS(=O)(=O)n5cccn5)nc(OC[C@@]56CCCN5C[C@H](F)C6)nc4c3F)c12]

Step A. 3-(methylamino)propane-1-sulfonic acid: To a solution of 1,2-oxathiolane 2,2-dioxide (10.0 g, 1.0 equiv) in THF (200 mL) was added MeNH₂ (2 M, 205 mL, 5.0 equiv) at 0 °C. The reaction was stirred at 20 °C for 12 hours. The mixture was concentrated and by re-crystallization from MeOH (200 mL) at 60 °C for 0.5 hours to afford the title compound (6.00 g, 48% yield) as white solid; ¹H NMR (400 MHz, D₂O) δ=3.18-3.13 (m, 2H), 2.99-2.95 (m, 2H), 2.70 (s, 3H), 2.12-2.08 (m, 2H).

Step B. 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (1.50 g, 1.0 equiv) and TEA (1.37 g, 5.0 equiv) in DMSO (10 mL) were added benzotriazol-1-yloxyl-tris-(pyrrolidino)-phosphonium hexafluorophosphate (2.82 g, 2.0 equiv) and 3-(methylamino)propane-1-sulfonic acid (829 mg, 2.0 equiv). The reaction was stirred at 20 °C for 12 hours. The mixture was filtered and purified by reversed phase flash [C18, 0.1% formic acid condition] to afford the title compound (1.60 g, 85% yield) as white solid; LCMS (ESI, M+1): m/z=690.2.

Step C. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid: To a solution of 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid (200 mg, 1.0 equiv) in DCM (1 mL) was added TFA (3.07 g, 93 equiv) at 0 °C. The reaction was stirred at 0 °C for 0.5 hours. The mixture was concentrated. The residue was added water (1 mL). The mixture was adjusted to pH=7 with NaHCO₃ and extracted with a mixture solution (EA/MeOH=4:1, 2×10 mL). The combined organic layers were concentrated to afford the title compound (130 mg, 66% yield) as off-white solid; LCMS (ESI, M+1): m/z=646.3.

Step D. 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonyl chloride: To a solution of 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonic acid (100 mg, 1.0 equiv) in DCM (5 mL) was added (COCl)₂ (59.0 mg, 3.0 equiv) at 0 °C. The reaction was stirred at 0 °C for 5 mins. DMF (1.13 mg, 0.1 equiv) was added above mixture. The reaction was stirred at 20 °C for 0.5 hours. The mixture was concentrated to afford the title compound (120 mg, crude) as yellow solid.

Step E. 4-(4-((3-((1H-pyrazol-1-yl)sulfonyl)propyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a mixture of 3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)propane-1-sulfonyl chloride (120 mg, crude) and 1H-pyrazole (61.5 mg, 5.0 equiv) in THF (6 mL) was added t-BuOK (60.8 mg, 3.0 equiv) at 0 °C. The reaction was stirred at 25 °C for 12 hours. The mixture was concentrated and purified by reversed phase flash [C18, H₂O/ACN condition] and reversed phase flash [C18, 0.1% formic acid condition] to afford the title compound (3.16 mg, 2% yield, 0.35 HCOOH) as white solid; ¹H NMR (400 MHz, DMSO-d₆) δ=10.65-9.43 (m, 1H), 9.24 (s, 1H), 8.36 (br s, 1H), 7.96 (s, 1H), 7.76 (br t, J=7.2 Hz, 1H), 7.40-7.29 (m, 2H), 7.00 (br s, 1H), 6.63 (br s, 1H), 5.40-5.17 (m, 1H), 4.17-4.10 (m, 1H), 4.07-4.02 (m, 1H), 3.92-3.82 (m, 4H), 3.52 (br s, 3H), 3.11 (br d, J=11.6 Hz, 2H), 3.03 (br s, 1H), 2.84 (br d, J=4.4 Hz, 1H), 2.39-2.31 (m, 1H), 2.20-2.10 (m, 2H), 2.07-1.95 (m, 4H), 1.88-1.73 (m, 3H), 0.73 (br t, J=6.8 Hz, 3H); LCMS (ESI, M+1): m/z=696.4.

### Example 231

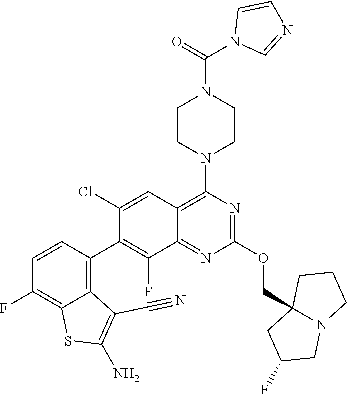 [SMILES: N#Cc1c(N)sc2c(F)ccc(-c3c(Cl)cc4c(N5CCN(C(=O)n6ccnc6)CC5)nc(OC[C@@]56CCCN5C[C@H](F)C6)nc4c3F)c12]

### 4-(4-(4-(1H-imidazole-1-carbonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

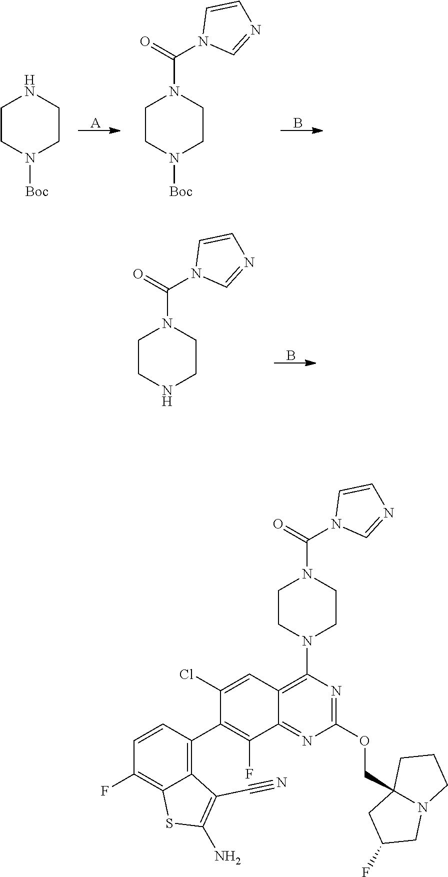

Step A. tert-butyl 4-(1H-imidazole-1-carbonyl)piperazine-1-carboxylate: To a solution of tert-butyl piperazine-1-carboxylate (800 mg, 1.0 equiv) in DCM (5 mL) was added CDI (557.18 mg, 0.8 equiv). The reaction was stirred at 0 °C for 0.5 hours. The mixture was concentrated and purified with reversed phase flash chromatography [C18, 0.1% NH₃·H₂O condition] to afford the title compound (1.0 g, 80% yield) as white solid. LCMS (ESI, M+1): m/z=281.2.

Step B. (1H-imidazol-1-yl) (piperazin-1-yl)methanone: To a solution of tert-butyl 4-(1H-imidazole-1-carbonyl)piperazine-1-carboxylate (600 mg, 1.0 equiv) in MeOH (6 mL) was added HCl·MeOH (2 M, 6.00 mL). The reaction was stirred at 25 °C for 0.5 hours. The mixture was concentrated to afford the title compound (300 mg, crude, HCl salt) as white solid; LCMS (ESI, M+1): m/z=181.2.

Step C. 4-(4-(4-(1H-imidazole-1-carbonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of (1H-imidazol-1-yl) (piperazin-1-yl)methanone (71.4 mg, 3 equiv, HCl), DIEA (142 mg, 10 equiv) and PyBOP (57.2 mg, 1.0 equiv) in DMSO (1 mL) was added 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (60 mg, 1.0 equiv). The reaction was stirred at 40 °C for 0.5 hours. The mixture was filtered and purified by reversed phase flash chromatography [Waters Xbridge 150×25 mm×5 μm; A: water (ammonia hydroxide v/v); B: ACN; B %: 28%-58% over 10 min], followed by prep-TLC [SiO₂, DCM:MeOH=1:1] to afford the title compound (13 mg, 16% yield) as white solid. ¹H NMR (400 MHz, CHLOROFORM-d) δ=7.95 (s, 1H), 7.73 (s, 1H), 7.26-7.25 (m, 1H), 7.22 (dd, J=5.2, 8.2 Hz, 1H), 7.15 (s, 1H), 7.04 (t, J=8.8 Hz, 1H), 5.58-5.46 (m, 2H), 5.41-5.20 (m, 1H), 4.36-4.19 (m, 2H), 4.01-3.80 (m, 8H), 3.45-3.16 (m, 3H), 3.11-2.94 (m, 1H), 2.34-2.14 (m, 3H), 2.05-1.91 (m, 3H); LCMS (ESI, M+1): m/z=708.3.

### Example 232

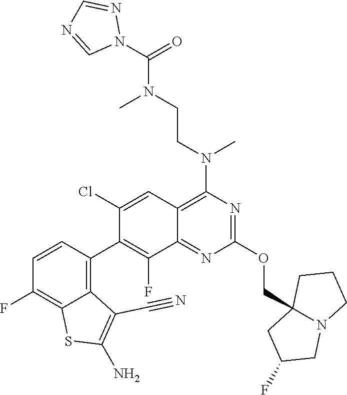 [SMILES: CN(CCN(C)c1nc(OC[C@@]23CCCN2C[C@H](F)C3)nc2c(F)c(-c3ccc(F)c4sc(N)c(C#N)c34)c(Cl)cc12)C(=O)n1cncn1]

### N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-1H-1,2,4-triazole-1-carboxamide

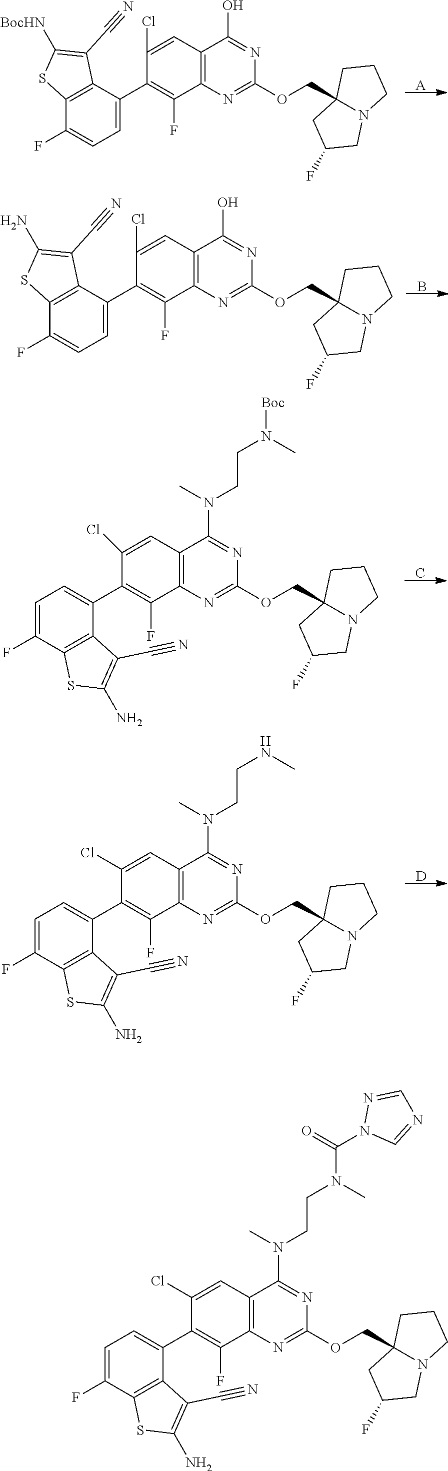

Step A. 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (1.00 g, 1.0 eq) in DCM (5 mL) was added TFA (2.5 mL) at 0 °C. The reaction was stirred at 0 °C for 2.5 hours. The mixture was concentrated. The residue diluter with DCM (10 mL), and the organic solution was adjusted to pH>7 with saturated sodium bicarbonate aqueous solution (50 mL), and then the mixed solution was extracted with a mixture solvent of dichloromethane (80 mL) and methanol (8 mL) for twice, and the combined organic phase were washed with brine (50 mL), dried over anhydrous sodium sulfate, concentrated to afford the title compound (760 mg, 90% yield) as a yellow solid; LCMS (ESI, M+1): m/z=546.1.

Step B. tert-butyl(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)(methyl)carbamate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (100 mg, 1.0 equiv) and tert-butyl N-methyl-N-[2-(methylamino)ethyl]carbamate (41.4 mg, 1.2 equiv) in N, N-Dimethylformamide (1.00 mL) were added benzotriazol-1-yloxy(tripyrrolidin-1-yl)phosphonium hexafluorophosphate (143 mg, 1.5 equiv) and diisopropylethylamine (71.0 mg, 3.0 equiv) at 25 °C. The reaction was stirred at 25 °C for 1 hour. The mixture was cooled to 15 °C and added to water (2 mL) slowly, filtered, and then the filter cake was dried in vacuum to afford the title compound (140 mg, crude) as a white solid; LCMS (ESI, M+1): m/z=716.4.

Step C. 2-amino-4-(6-chloro-8-fluoro-2-(((2R,7aS)-2-fluorotetrahydro-1H-pyrrolizin-7a(5H)-yl)methoxy)-4-(methyl(2-(methylamino)ethyl)amino)quinazolin-7-yl)-7-fluorobenzo[b]thiophene-3-carbonitrile: To a solution of tert-butyl(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)(methyl)carbamate (140 mg, 1.0 equiv) in acetonitrile (1.5 mL) was added HCl·dioxane (2 M, 0.44 mL, 4.5 equiv) at 0 °C. The reaction was stirred at 25 °C for 1 hour. The mixture was directly concentrated to give a residue. The residue was diluted with methanol (10 mL) and adjusted to pH=8 with sodium bicarbonate solid, filtered, and the filtrate was directly concentrated to afford the title compound (120 mg, 99% yield) as a yellow solid; LCMS (ESI, M+1): m/z=616.3.

Step D. N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-N-methyl-1H-1,2,4-triazole-1-carboxamide: To a solution of 2-amino-4-(6-chloro-8-fluoro-2-(((2R,7aS)-2-fluorotetrahydro-1H-pyrrolizin-7a(5H)-yl)methoxy)-4-(methyl(2-(methylamino)ethyl)amino)quinazolin-7-yl)-7-fluorobenzo[b]thiophene-3-carbonitrile (50.0 mg, 1.0 equiv) in dichloromethane (0.6 mL) were added di(1H-1,2,4-triazol-1-yl)methanone (14.7 mg, 1.1 equiv) and diisopropylethylamine (31.5 mg, 3.0 equiv) at 20 °C. The reaction was stirred at 20 °C for 1 hour. The mixture was directly concentrated and purified by prep-HPLC [Waters Xbridge 150× 25 mm×5 μm; A: water (10 mM NH₄HCO₃), B: ACN, B %: 42% to 72% over 9 min] and further purified by prep-HPLC [Welch Xtimate C18 150×25 mm×5 μm; A: water (0.1% FA), B: ACN; B %: 20% to 40% over 10 min] to afford the title compound (5.50 mg, 9.3% yield) as a white solid; ¹H NMR (400 MHz, methanol-d₄) δ=8.85 (d, J=2.8 Hz, 1H), 8.11 (br s, 1H), 8.07 (s, 1H), 7.21 (dd, J=5.2, 8.4 Hz, 1H), 7.04 (t, J=8.8 Hz, 1H), 5.47-5.26 (m, 1H), 4.53-3.82 (m, 7H), 3.79-3.34 (m, 8H), 3.22-3.10 (m, 1H), 2.38-1.87 (m, 6H); LCMS (ESI, M+1): m/z=711.2.

### Example 233

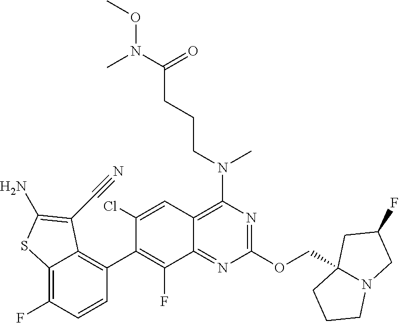 [SMILES: CON(C)C(=O)CCCN(C)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methoxy-N-methylbutanamide

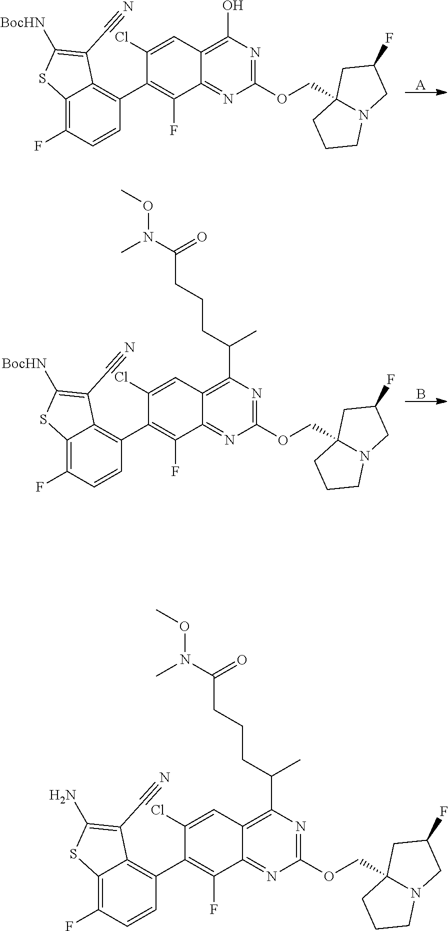

Step A. tert-butyl(4-(6-chloro-8-fluoro-2-(((2R,7aS)-2-fluorotetrahydro-1H-pyrrolizin-7a(5H)-yl)methoxy)-4-((4-(methoxy(methyl)amino)-4-oxobutyl)(methyl)amino)quinazolin-7-yl)-3-cyano-7-fluorobenzo[b]thiophen-2-yl)carbamate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (184 mg, 1.0 eq.), PyBOP (178 mg, 1.2 eq.) and DIEA (111 mg, 3.0 eq.) in DMF (1 mL) were added N-methoxy-N-methyl-4-(methylamino)butanamide (102 mg, 1.3 equiv, TFA) and DIEA (111 mg, 3.0 eq.). The reaction was stirred at 25 °C for 0.5 hour. The mixture was filtered and purified with prep-HPLC [Phenomenex luna C18 150×40 mm 15 μm; A: water (HCOOH); B: ACN, B %: 28%-58% over 22 min) to afford the title compound (120 mg, 52% yield) as a white solid; LCMS (ESI, M+1): m/z=788.3.

Step B. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methoxy-N-methylbutanamide: To a solution of tert-butyl(4-(6-chloro-8-fluoro-2-(((2R,7aS)-2-fluorotetrahydro-1H-pyrrolizin-7a(5H)-yl)methoxy)-4-((4-(methoxy(methyl)amino)-4-oxobutyl)(methyl)amino)quinazolin-7-yl)-3-cyano-7-fluorobenzo[b]thiophen-2-yl)carbamate (100 mg, 1.0 eq.) in DCM (2 mL) was added TFA (2.00 mL, 212 eq.) slowly at 0 °C. The reaction was stirred at 25 °C for 1 hour. The mixture was concentrated, redissolved in MeOH (3 mL), neutralized with NaHCO₃solid, filtered and concentrated. The residue was purified with prep-HPLC [Phenomenex luna C18 150×25 mm×10 μm; A: water (HCOOH); B: ACN, B %: 23%-53% over 11 min) to afford the title compound (22.4 mg, 26% yield) as a white solid; ¹H NMR (400 MHz, METHANOL-d4) δ=8.16 (s, 1H), 7.26-7.17 (m, 1H), 7.10-7.01 (m, 1H), 5.80-5.31 (m, 1H), 4.71-4.52 (m, 2H), 4.03-3.74 (m, 5H), 3.70 (s, 3H), 3.57 (s, 3H), 3.43-3.35 (m, 1H), 3.17 (s, 3H), 2.50 (br s, 4H), 2.47-2.35 (m, 1H), 2.34-2.22 (m, 2H), 2.21-2.01 (m, 3H); LCMS (ESI, M+1): m/z=688.3.

### Example 234

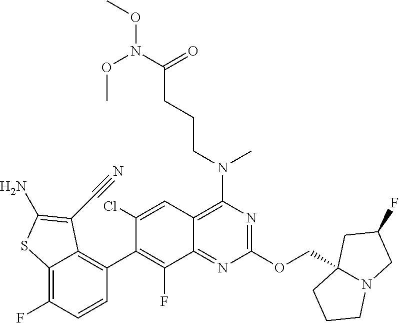 [SMILES: CON(OC)C(=O)CCCN(C)c1nc(OC[C@@]23CCCN2C[C@H](F)C3)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methoxybutanamide

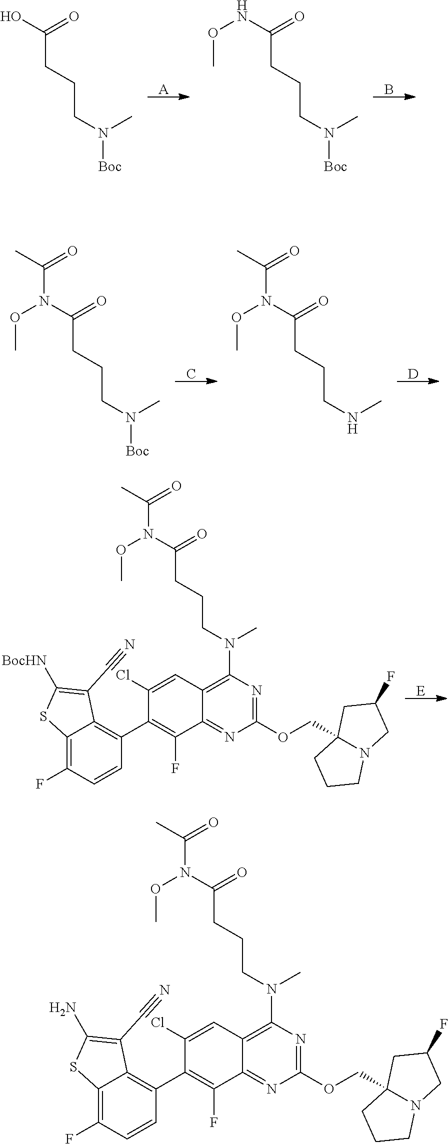

Step A. tert-butyl(4-(methoxyamino)-4-oxobutyl)(methyl)carbamate: To a solution of 4-((tert-butoxycarbonyl)(methyl)amino)butanoic acid (2.00 g, 1.0 eq.) and MeONH₂·HCl (1.15 g, 1.5 eq.) in DCM (20 mL) were added EDCI (2.65 g, 1.5 eq.) and DMAP (3.37 g, 3.0 eq.). The reaction was stirred at 20 °C for 3 hours. The mixture was concentrated and purified with prep-HPLC [column: Waters Xbridge Prep OBD C18 150×40 mm×10 μm; mobile phase: water (NH₄HCO₃)-ACN; gradient: 6%-36% B over 15 min] to afford the title compound (2.09 g, 87%) as colorless oil; LCMS (ESI, M−99): m/z=147.3.

Step B. tert-butyl(4-(N-methoxyacetamido)-4-oxobutyl)(methyl)carbamate: To a solution of tert-butyl(4-(methoxyamino)-4-oxobutyl)(methyl)carbamate (2.00 g, 1 eq.) in THF (20 mL) was added NaH (357 mg, 1.1 equiv, 60% purity) portion wise at 0 °C under nitrogen atmosphere. The reaction was stirred at 0 °C for 0.2 hours and then acetic anhydride (995 mg, 1.2 eq.) was added. The reaction was stirred at 25 °C for 12 hours. The mixture was quenched with water (30 mL) under nitrogen atmosphere at 0 °C and extracted with ethyl acetate (3×20 mL). The combined organic layers were washed with brine (30 mL), dried over anhydrous sodium sulfate, concentrated, and purified with column chromatography [SiO₂, petroleum ether/ethyl acetate=10/1 to 2/1] to afford the title compound (2.16 g, 89% yield) as colorless oil; LCMS (ESI, M+23): m/z=311.1.

Step C. N-acetyl-N-methoxy-4-(methylamino)butanamide: To a solution of tert-butyl(4-(N-methoxyacetamido)-4-oxobutyl)(methyl)carbamate (300 mg, 1.0 eq.) in DCM (3 mL) was added TFA (1 mL). The reaction was stirred at 25 °C for 0.5 hour. The mixture was concentrated to afford the title compound (195 mg, crude) as colorless oil.

Step D. tert-butyl(4-(6-chloro-8-fluoro-2-(((2R,7aS)-2-fluorotetrahydro-1H-pyrrolizin-7a(5H)-yl)methoxy)-4-((4-(N-methoxyacetamido)-4-oxobutyl)(methyl)amino)quinazolin-7-yl)-3-cyano-7-fluorobenzo[b]thiophen-2-yl)carbamate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (167 mg, 1.0 eq.) in DMF (4 mL) were added PyBOP (202 mg, 1.5 eq.) and DIEA (167 mg, 5.0 eq.). The reaction was stirred at 25 °C for 0.5 hour and then N-acetyl-N-methoxy-4-(methylamino)butanamide (195 mg, 4.0 eq.) was added. The reaction was stirred at 25 °C for 1 hour. The mixture was filtered and purified with prep-HPLC [column: Waters Xbridge Prep OBD C18 150×40 mm×10 μm; mobile phase: water (NH₄HCO₃)-ACN; gradient: 52%-82% B over 15 min] to afford the title compound (84.0 mg, 35% yield) as yellow oil; LCMS (ESI, M+1): m/z=816.4.

Step E. N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methoxybutanamide: To a solution of tert-butyl(4-(6-chloro-8-fluoro-2-(((2R,7aS)-2-fluorotetrahydro-1H-pyrrolizin-7a(5H)-yl)methoxy)-4-((4-(N-methoxyacetamido)-4-oxobutyl)(methyl)amino)quinazolin-7-yl)-3-cyano-7-fluorobenzo[b]thiophen-2-yl)carbamate (45.0 mg, 1.0 eq.) in DCM (1.5 mL) was added TFA (0.5 mL). The reaction was stirred at 25 °C for 0.5 hour. The mixture was concentrated, diluted with ethyl acetate (5 mL) and neutralized with DIEA at −40 °C The organic layer were washed with water (5 mL), dried over anhydrous sodium sulfate, concentrated and purified with prep-HPLC [column: Waters Xbridge 150×25 mm×5 μm; mobile phase: water (NH₄HCO₃)-ACN; gradient: 48%-78% B over 10 min] to afford the title compound (9.87 mg, 24% yield,) as off-white solid; ¹H NMR (400 MHz, DMSO-d₆) δ=8.15-8.03 (m, 3H), 7.24 (dd, J=5.2, 8.4 Hz, 1H), 7.18-7.11 (m, 1H), 5.36-5.18 (m, 1H), 4.12-3.98 (m, 2H), 3.88-3.77 (m, 2H), 3.74 (s, 3H), 3.42 (s, 3H), 3.14-3.04 (m, 2H), 3.00 (s, 1H), 2.86-2.77 (m, 3H), 2.32 (s, 3H), 2.14-1.98 (m, 5H), 1.86-1.73 (m, 3H); LCMS (ESI, M+1): m/z=716.4.

### Example 235

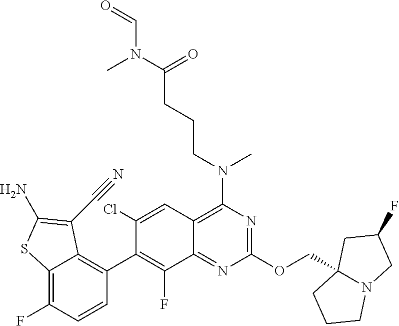 [SMILES: CN(C=O)C(=O)CCCN(C)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-formyl-N-methylbutanamide

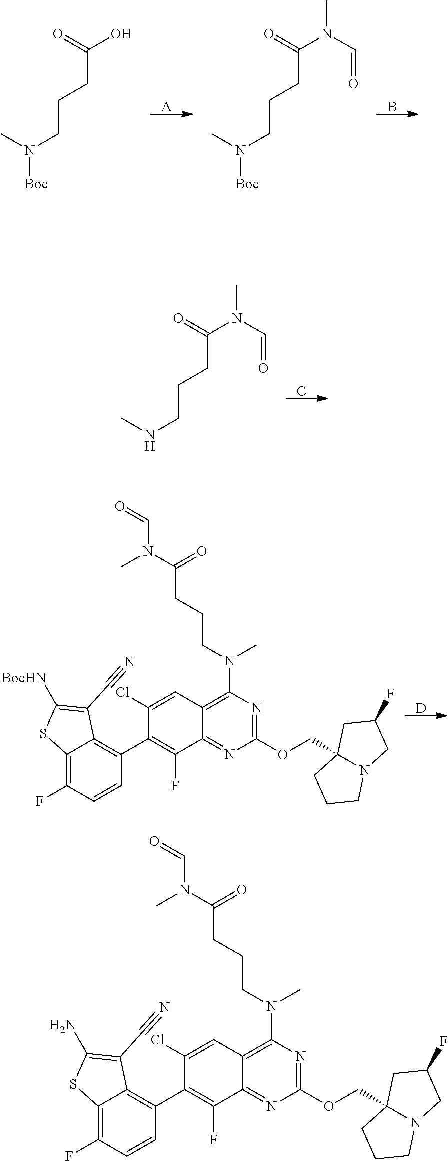

Step A. tert-butyl methyl(4-(N-methylformamido)-4-oxobutyl)carbamate: To a solution of 4-((tert-butoxycarbonyl)(methyl)amino)butanoic acid (500 mg, 1.0 eq.) in THF (3 mL) were added isobutyl carbonochloridate (314 mg, 1.0 eq.) and 4-methylmorpholine (256 mg, 1.1 eq.). After addition, the reaction was stirred at 0 °C for 0.03 hours and then N-methylformamide (163 mg, 1.2 eq.) was added. The reaction was stirred at 25 °C for 1 hour. The mixture was diluted with water (10 mL) and extracted with ethyl acetate (3×10 mL). The combined organic layers were washed with brine (10 mL), dried over anhydrous sodium sulfate, concentrated and purified with column chromatography [SiO₂, petroleum ether/ethyl acetate=50/1 to 10/1] to afford the title compound (320 mg, 54% yield) as yellow solid; ¹H NMR (400 MHz, CHLOROFORM-d) δ=9.29-9.24 (m, 1H), 3.32 (t, J=6.8 Hz, 2H), 3.14-3.11 (m, 3H), 2.86 (s, 3H), 2.63 (t, J=6.8 Hz, 2H), 1.95 (quin, J=7.2 Hz, 2H), 1.44 (s, 9H).

Step B. N-formyl-N-methyl-4-(methylamino)butanamide: To a solution of tert-butyl methyl(4-(N-methylformamido)-4-oxobutyl)carbamate (320 mg, 1.0 eq.) in DCM (3 mL) was added TFA (1 mL). The reaction was stirred at 25 °C for 1 hour. The mixture was concentrated, dissolved in MeCN (2 mL), neutralized with DIEA at −40 °C and concentrated to afford the title compound (180 mg, crude) as yellow oil.

Step C. tert-butyl(4-(6-chloro-8-fluoro-2-(((2R,7aS)-2-fluorotetrahydro-1H-pyrrolizin-7a(5H)-yl)methoxy)-4-(methyl(4-(N-methylformamido)-4-oxobutyl)amino)quinazolin-7-yl)-3-cyano-7-fluorobenzo[b]thiophen-2-yl)carbamate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (200 mg, 1.0 eq.) and N-formyl-N-methyl-4-(methylamino)butanamide (171 mg, 3.5 eq.) in DMF (4 mL) were added PyBOP (242 mg, 1.5 eq.) and DIEA (200 mg, 5.0 eq.). The reaction was stirred at 40 °C for 1 hour. The mixture was diluted with water (20 mL) and extracted with ethyl acetate (3×10 mL). The combined organic layers were washed with brine (20 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 52%-82% B over 15 min] to afford the title compound (100 mg, 41% yield) as yellow solid; LCMS (ESI, M+1): m/z=786.1.

Step D. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-formyl-N-methylbutanamide: To a solution of tert-butyl(4-(6-chloro-8-fluoro-2-(((2S,3aR)-2-fluorohexahydropentalen-3a(1H)-yl)methoxy)-4-(methyl(4-(N-methylformamido)-4-oxobutyl)amino)quinazolin-7-yl)-3-cyano-7-fluorobenzo[b]thiophen-2-yl)carbamate (50.0 mg, 1.0 eq.) in DCM (3 mL) was added TFA (1 mL). The reaction was stirred at 25 °C for 1 hour. The mixture was concentrated, redissolved in MeCN (2 mL) and neutralized with DIEA at −40 °C The mixture was diluted with water (10 mL) and extracted with ethyl acetate (3×10 mL). The combined organic layers were washed with brine (10 mL), dried over anhydrous sodium sulfate, concentrated and purified with prep-HPLC [column: Waters Xbridge 150×25 mm×5 μm; mobile phase: water (NH₄HCO₃)-ACN; gradient: 48%-78% B over 9 min] to afford the title compound (9.74 mg, 21% yield) as pink solid; ¹H NMR (400 MHz, DMSO-d₆) δ=9.24 (s, 1H), 8.14-8.04 (m, 3H), 7.24 (dd, J=5.2, 8.0 Hz, 1H), 7.18-7.11 (m, 1H), 5.41-5.14 (m, 1H), 4.12-4.06 (m, 1H), 4.04-3.96 (m, 1H), 3.88-3.70 (m, 2H), 3.43 (s, 3H), 3.14-3.05 (m, 2H), 3.04-2.99 (m, 1H), 2.94 (s, 3H), 2.89 (t, J=7.2 Hz, 2H), 2.85-2.78 (m, 1H), 2.14-1.99 (m, 5H), 1.86-1.72 (m, 3H); LCMS (ESI, M+1): m/z=686.1.

### Example 236

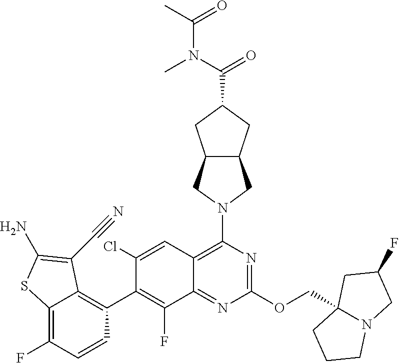

### (3aR,5S,6aS)-N-acetyl-2-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methyloctahydrocyclopenta[c]pyrrole-5-carboxamide

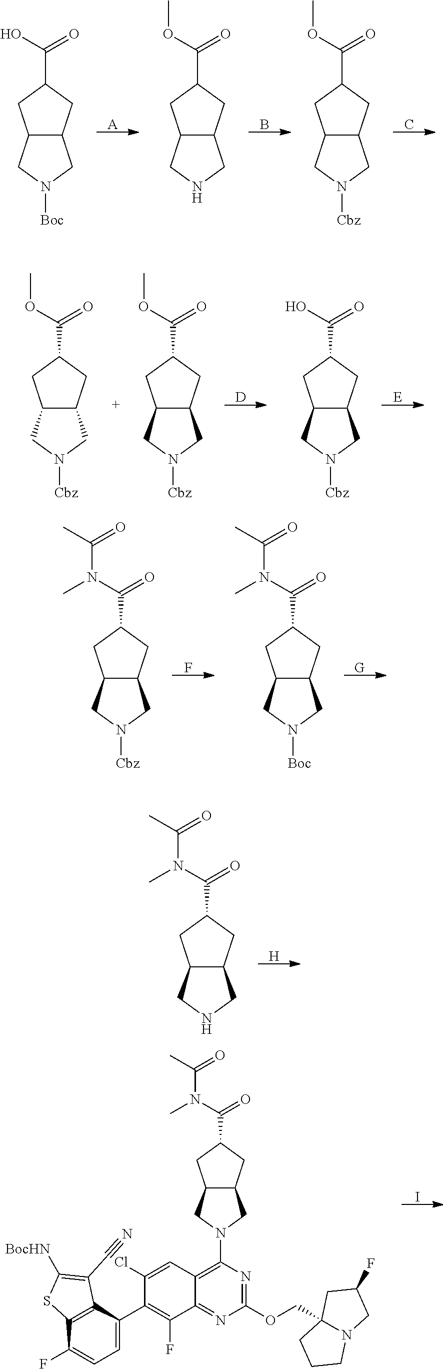

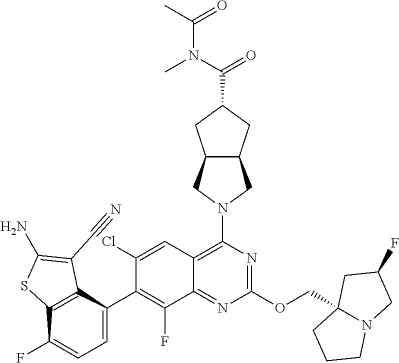

Step A. methyl octahydrocyclopenta[c]pyrrole-5-carboxylate: To a solution of 2-(tert-butoxycarbonyl)octahydrocyclopenta[c]pyrrole-5-carboxylic acid (6.00 g, 1.0 eq.) in MeOH (10 mL) was added HCl·MeOH (2 M, 2.5 eq.) at 25 °C. The reaction was stirred at 25 °C for 2 hours. The mixture was concentrated under vacuum to afford the title compound (3.9 g, crude) as a yellow solid; LCMS (ESI, M+1): m/z=170.0.

Step B. 2-benzyl 5-methyl hexahydrocyclopenta[c]pyrrole-2,5 (1H)-dicarboxylate: To a solution of methyl octahydrocyclopenta[c]pyrrole-5-carboxylate (3.89 g, 1.0 eq.) and DIEA (14.8 g, 5.0 eq.) in DCM (40 mL) was added CbzCl (5.88 g, 1.5 eq.) at 25 °C. The mixture was stirred at 25 °C for 2 hours. The mixture was concentrated and purified with reversed phase flash chromatography (0.1% FA condition) to afford the title compound (6.00 g, 81% yield) as a yellow solid. LCMS (ESI, M+1): m/z=304.2.

Step C. 2-benzyl 5-methyl(3aR,5s,6aS)-hexahydrocyclopenta[c]pyrrole-2,5 (1H)-dicarboxylate: Purified by SFC separation [column: DAICEL CHIRALPAK IG (250 mm×50 mm, 10 μm); mobile phase: [CO₂-i-PrOH (0.1% NH₃H₂O)]; B %: 50%, isocratic elution mode] to afford two isomers.

Peak 1:2-benzyl 5-methyl(3aR,5s,6aS)-hexahydrocyclopenta[c]pyrrole-2,5 (1H)-dicarboxylate (900 mg, 14% yield) as yellow oil. ¹H NMR (400 MHz, DMSO-d6) δ=7.36-7.07 (m, 5H), 5.04 (s, 2H), 3.59 (s, 3H), 3.56-3.46 (m, 2H), 3.11 (br d, J=10.4 Hz, 2H), 2.97 (quin, J=8.0 Hz, 1H), 2.73 (br d, J=2.4 Hz, 2H), 1.94 (td, J=8.0, 13.3 Hz, 2H), 1.73 (ddd, J=3.2, 8.0, 13.2 Hz, 2H); LCMS (ESI, M+1): m/z=441.2

Peak 2:2-benzyl 5-methyl(3aR,5r,6aS)-hexahydrocyclopenta[c]pyrrole-2,5 (1H)-dicarboxylate: (3.8 g, 61% yield) as yellow oil. ¹H NMR (400 MHz, DMSO-d₆) δ=7.38-7.25 (m, 5H), 5.05 (s, 2H), 3.58 (s, 3H), 3.44 (br d, J=4.4 Hz, 2H), 3.22 (br s, 2H), 2.94-2.82 (m, 1H), 2.63 (br s, 2H), 2.12 (td, J=8.0, 13.2 Hz, 2H), 1.54 (ddd, J=7.2, 9.7, 12.9 Hz, 2H); LCMS (ESI, M+1): m/z=441.2

Step D. (3aR,5s,6aS)-2-((benzyloxy)carbonyl)octahydrocyclopenta[c]pyrrole-5-carboxylic acid: To a solution of 2-benzyl 5-methyl(3aR,5s,6aS)-hexahydrocyclopenta[c]pyrrole-2,5 (1H)-dicarboxylate (500 mg, 1.0 eq.) in THF (5.0 mL) was added LiOH·H₂O (1.5 M, 0.9 eq.) at 25 °C. The reaction was stirred at 25-30 °C for 1 hour. The mixture was acidified with citric acid aqueous solution (2.0 mL) and extracted with ethyl acetate (3×6.0 mL). The organic layer was dried over anhydrous sodium sulfate and concentrated to afford the title compound (400 mg, crude) as a yellow solid. LCMS (ESI, M+1): m/z=290.1

Step E: benzyl(3aR,5s,6aS)-5-(acetyl(methyl) carbamoyl)hexahydrocyclopenta[c]pyrrole-2 (1H)-carboxylate: To a solution of (3aR,5s,6aS)-2-((benzyloxy)carbonyl)octahydrocyclopenta[c]pyrrole-5-carboxylic acid (350 mg, 1.0 eq.) and N-methylacetamide (133 mg, 1.5 eq.) in 2-MeTHF (3.5 mL) were added CMPI (309 mg, 1.0 eq.) and DIEA (469 mg, 3.0 eq.) at 25 °C. The reaction was stirred at 80 °C for 2 hours. The mixture was diluted with water (2.0 mL) and extracted with ethyl acetate (3×3.0 mL). The combined organic layers were dried over anhydrous sodium sulfate, concentrated and purified by HPLC (0.1% FA condition) to afford the title compound (150 mg, crude) as a yellow solid. LCMS (ESI, M+1): m/z=345.2

Step F: tert-butyl(3aR,5s,6aS)-5-(acetyl(methyl) carbamoyl)hexahydrocyclopenta[c]pyrrole-2 (1H)-carboxylate: To the suspension of Pd/C (20.00 mg, 10% purity, 0.1 eq.) in EtOAc (1 mL) was added benzyl (3aR,5s,6aS)-5-(acetyl(methyl) carbamoyl)hexahydrocyclopenta[c]pyrrole-2 (1H)-carboxylate (80.00 mg, 1.0 eq.) and (Boc) 20 (50.7 mg, 1.0 eq.) under N₂. The reaction was degassed and purged with N₂ and H₂ for 3 times, and then stirred at 25 °C for 1 hour under H₂ atmosphere (15 Psi). The mixture was filtered and concentrated to afford the title compound (70 mg, crude) as a yellow solid.

Step G: (3aR,5s,6aS)-N-acetyl-N-methyloctahydrocyclopenta[c]pyrrole-5-carboxamide: To a solution of tert-butyl(3aR,5s,6aS)-5-(acetyl(methyl) carbamoyl)hexahydrocyclopenta[c]pyrrole-2 (1H)-carboxylate (60.0 mg, 1.0 eq.) in DCM (1.0 mL) was added TFA (1.54 g, 70 eq.) at 25 °C. The mixture was stirred at 25 °C for 1 hour. The mixture was concentrated to afford the title compound (40 mg, crude) as a yellow oil. LCMS (ESI, M+1): m/z=211.1

Step H: tert-butyl(4-((S)-4-((3aR,5S,6aS)-5-(acetyl(methyl) carbamoyl)hexahydrocyclopenta[c]pyrrol-2 (1H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (80 mg, 1.0 eq.) and (3aR,5s,6aS)-N-acetyl-N-methyloctahydrocyclopenta[c]pyrrole-5-carboxamide (33.8 mg, 1.3 eq.) in DMF (1.0 mL) were added PYBOP (64.4 mg, 1.0 eq.) and DIEA (48.0 mg, 3.0 eq.) at 25 °C. The mixture was stirred at 25 °C for 1 hour. The mixture was basified by saturated with NaHCO₃ (2.0 mL) and extracted with ethyl acetate (3×2.0 mL). The combined organic layer was dried over anhydrous sodium sulfate, concentrated, and purified with HPLC (0.1% FA condition) to afford the title compound (80 mg, 72% yield) as a yellow oil. LCMS (ESI, M+1): m/z=838.4

Step I: (3aR,5S,6aS)-N-acetyl-2-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methyloctahydrocyclopenta[c]pyrrole-5-carboxamide: To a solution of tert-butyl(4-((S)-4-((3aR,5S,6aS)-5-(acetyl(methyl) carbamoyl)hexahydrocyclopenta[c]pyrrol-2 (1H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (50.0 mg, 1.0 eq.) in DCM (0.5 mL) was added TFA (767 mg, 113 eq.) at 0 °C. The reaction was stirred at 25 °C for 1 hour. The mixture was diluted with water (2.0 mL) and extracted with ethyl acetate (3×2.0 mL). The combined organic layer was dried over anhydrous sodium sulfate, concentrated, and purified by HPLC [YMC-Actus Triart C18 150×30 mm×7 μm; A: water (FA), B: ACN, B %: 20%-50% over 15 min] to afford the title compound (28.6 mg, 64% yield) as a yellow oil. ¹H NMR (400 MHz, DMSO-d6) δ=8.22-8.06 (m, 3H), 7.29-7.08 (m, 2H), 5.45-5.14 (m, 1H), 4.23-3.95 (m, 4H), 3.92-3.74 (m, 3H), 3.15 (s, 3H), 3.14-2.94 (m, 3H), 2.93-2.80 (m, 3H), 2.37-2.31 (m, 3H), 2.20-2.06 (m, 3H), 2.05 (br d, J=7.6 Hz, 2H), 1.99-1.66 (m, 5H);LCMS (ESI, M+1): m/z=738.2.

### Example 237

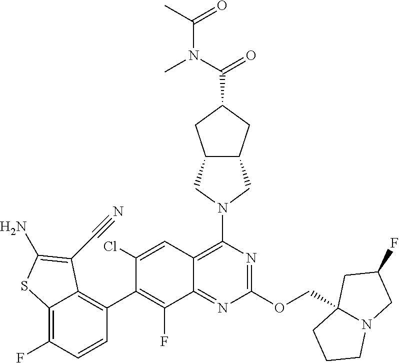 [SMILES: CC(=O)N(C)C(=O)[C@@H]1C[C@@H]2CN(c3nc(OC[C@@]45CCCN4C[C@H](F)C5)nc4c(F)c(-c5ccc(F)c6sc(N)c(C#N)c56)c(Cl)cc34)C[C@@H]2C1]

### (3aR,5R,6aS)-N-acetyl-2-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methyloctahydrocyclopenta[c]pyrrole-5-carboxamide

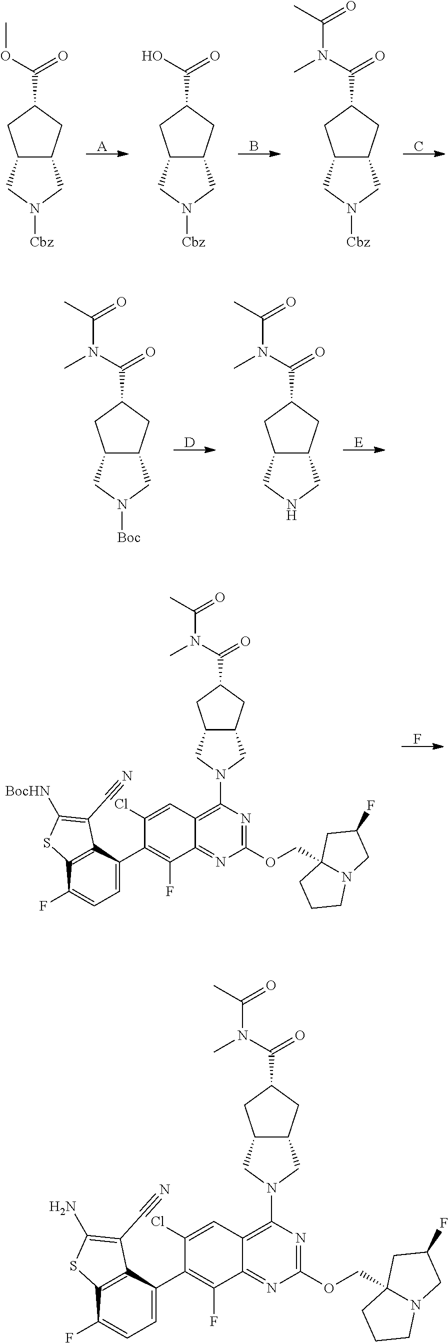

Step A. (3aR,5r,6aS)-2-((benzyloxy)carbonyl)octahydrocyclopenta[c]pyrrole-5-carboxylic acid: To a solution of 2-benzyl 5-methyl(3aR,5r,6aS)-hexahydrocyclopenta[c]pyrrole-2,5 (1H)-dicarboxylate (500 mg, 1.0 eq.) in THF (5.0 mL) was added LiOH·H₂O (1.5 M, 1.0 eq.) at 25 °C The reaction was stirred at 25-30 °C for 1 hour. The mixture was diluted with citric acid aqueous solution (2.0 mL) and extracted with ethyl acetate (3×6.0 mL). The organic layer was dried over anhydrous sodium sulfate and concentrated to afford the title compound (500 mg, 82% yield) as yellow solid; LCMS (ESI, M+1): m/z=290.1.

Step B. benzyl(3aR,5r,6aS)-5-(acetyl(methyl) carbamoyl)hexahydrocyclopenta[c]pyrrole-2 (1H)-carboxylate: To a solution of (3aR,5r,6aS)-2-((benzyloxy)carbonyl)octahydrocyclopenta[c]pyrrole-5-carboxylic acid (450 mg, 1.0 eq.) and N-methylacetamide (113 mg, 1.2 eq.) in 2-MeTHF (3.5 mL) were added CMPI (390 mg, 1.0 eq.) and DIEA (469 mg, 3.0 eq.) at 25 °C. The reaction was stirred at 80 °C for 2 hours. The mixture was diluted with water (2.0 mL) and extracted with ethyl acetate (3×3.0 mL). The combined organic layer was dried over anhydrous sodium sulfate, concentrated, and purified with reversed phase flash chromatography (0.1% formic acid condition) to afford the title compound (380 mg, 67% yield) as yellow solid; LCMS (ESI, M+1): m/z=345.1.

Step C. tert-butyl(3aR,5r,6aS)-5-(acetyl(methyl) carbamoyl)hexahydrocyclopenta[c]pyrrole-2 (1H)-carboxylate: To the suspension of Pd/C (20 mg, 10% purity) in EtOAc (1 mL) was added benzyl(3aR,5r,6aS)-5-(acetyl(methyl)carbamoyl)hexahydrocyclopenta[c]pyrrole-2 (1H)-carboxylate (80 mg, 1.0 eq.) and Boc₂O (50.7 mg, 1.0 eq.) under nitrogen. The mixture was degassed and purged with N₂ and H₂ for 3 times. The reaction was stirred at 25 °C for 1 hour under H₂ atmosphere (15 Psi). The mixture was filtered and concentrated to afford the title compound (70 mg, crude) as yellow solid; LCMS (ESI, M+1): m/z=311.2.

Step D. (3aR,5,6aS)-N-acetyl-N-methyloctahydrocyclopenta[c]pyrrole-5-carboxamide: To a solution of tert-butyl(3aR,5r,6aS)-5-(acetyl(methyl)carbamoyl)hexahydrocyclopenta[c]pyrrole-2 (1H)-carboxylate (60.0 mg, 1.0 eq.) in DCM (1.0 mL) was added TFA (1.54 g, 70 eq.) at 25 °C. The reaction was stirred at 25 °C for 1 hour. The mixture was concentrated to afford the title compound (40 mg, crude) as yellow oil; LCMS (ESI, M+1): m/z=211.1.

Step E tert-butyl(4-((S)-4-((3aR,5R,6aS)-5-(acetyl(methyl)carbamoyl)hexahydrocyclopenta[c]pyrrol-2 (1H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl(4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (80 mg, 1.0 eq.) and (3aR,5r,6aS)-N-acetyl-N-methyloctahydrocyclopenta[c]pyrrole-5-carboxamide (33.8 mg, 1.3 eq.) in DMF (1.0 mL) were added PyBOP (64.4 mg, 1.0 eq.) and DIEA (48.0 mg, 3.0 eq.) at 25 °C. The reaction was stirred at 25 °C for 1 hour. The mixture was diluted with water (2.0 mL) and extracted with ethyl acetate (3×2.0 mL). The combined organic layer was dried over anhydrous sodium sulfate, concentrated and purified with reversed phase flash chromatography (0.1% formic acid condition) to afford the title compound (100 mg, 74% yield) as yellow oil; LCMS (ESI, M+1): m/z=838.2.

Step F (3aR,5R,6aS)-N-acetyl-2-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methyloctahydrocyclopenta[c]pyrrole-5-carboxamide: To a solution of tert-butyl(4-((S)-4-((3aR,5R,6aS)-5-(acetyl(methyl)carbamoyl)hexahydrocyclopenta[c]pyrrol-2 (1H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (50.0 mg, 1.0 eq.) in DCM (0.5 mL) was added TFA (767 mg, 113 eq.) at 0 °C. The mixture was stirred at 25 °C for 1 hour. The reaction was basified by NaHCO₃ (2.0 mL) and extracted with ethyl acetate (3×2.0 mL). The organic layer was dried over anhydrous sodium sulfate, filtered and concentrated and purified with prep-HPLC [YMC-Actus Triart C18 150×30 mm×7 μm; A: water (FA), B: ACN, B %: 20%-50% over 15 min] to afford the title compound (24.0 mg, 33% yield) as yellow oil; ¹H NMR (400 MHz, DIMETHYL SULFOXIDE-d₆) δ=8.13 (s, 3H), 7.29-7.09 (m, 2H), 5.40-5.13 (m, 1H), 4.22-3.93 (m, 4H), 3.83 (br t, J=8.4 Hz, 2H), 3.70 (t, J=8.8 Hz, 1H), 3.15 (s, 3H), 3.11-2.97 (m, 3H), 2.82 (br d, J=6.0 Hz, 3H), 2.33 (s, 3H), 2.27-2.10 (m, 3H), 2.08-1.97 (m, 2H), 1.88-1.71 (m, 5H); LCMS (ESI, M+1): m/z=738.2.

### Example 238

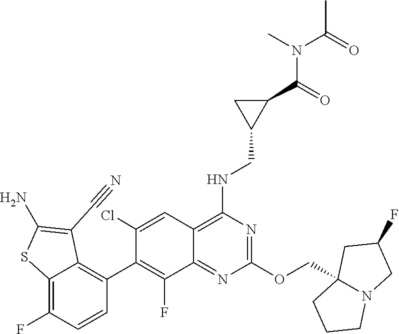 [SMILES: CC(=O)N(C)C(=O)[C@@H]1C[C@H]1CNc1nc(OC[C@@]23CCCN2C[C@H](F)C3)nc2c(F)c(-c3ccc(F)c4sc(N)c(C#N)c34)c(Cl)cc12]

### trans-N-acetyl-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)-N-methylcyclopropane-1-carboxamide

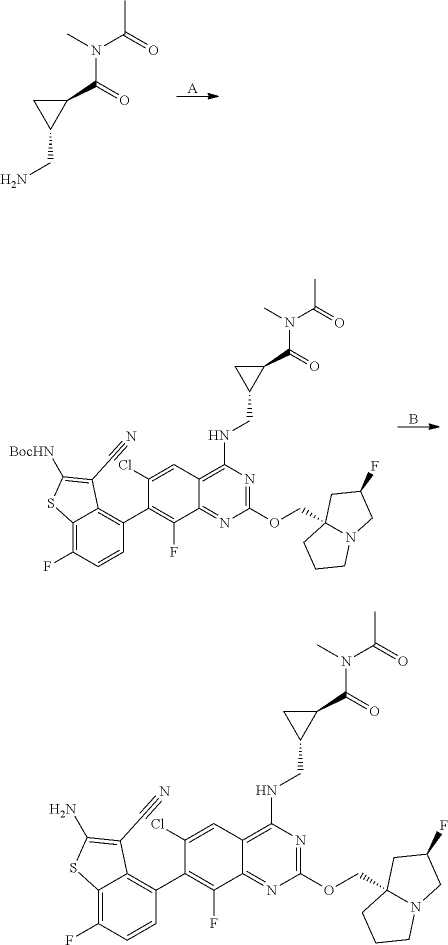

Step A. tert-butyl(4-(4-((((1R,2R)-2-(acetyl(methyl)carbamoyl)cyclopropyl)methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of trans-N-acetyl-2-(aminomethyl)-N-methylcyclopropane-1-carboxamide (30.0 mg, 2.0 eq.) in DMF (2 mL) were added tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (56.9 mg, 1.0 eq.), DIEA (56.9 mg, 5.0 eq.) and PyBOP (68.8 mg, 1.5 eq.). The reaction was stirred at 40 °C for 1 hour. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 55%-85% B over 52 min] to afford the title compound (30.0 mg, 43% yield) as yellow solid; LCMS (ESI, M+1): m/z=798.3.

Step B. (1R,2R)-N-acetyl-2-(((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)methyl)-N-methylcyclopropane-1-carboxamide: To a solution of tert-butyl(4-(4-((((1R,2R)-2-(acetyl(methyl)carbamoyl)cyclopropyl)methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (28.0 mg, 1.0 eq.) in DCM (3 mL) was added TFA (1 mL). The reaction was stirred at 25 °C for 1 hour. The pH of the mixture was adjusted to 9-10 with DIEA at −40 °C, diluted with water (10 mL) and extracted with DCM (2×10 mL). The combined organic layers were washed with brine (10 mL), dried over anhydrous sodium sulfate, concentrated and purified with prep-HPLC [column: Waters Xbridge BEH C18 150×25 mm×5 μm; mobile phase: water (NH₄HCO₃)-ACN; gradient: 38%-68% B over 9 min] to afford the title compound (8.87 mg, 35% yield) as white solid; ¹H NMR (400 MHz, METHANOL-d₄) δ=8.15 (s, 1H), 7.23-7.16 (m, 1H), 7.03 (t, J=8.8 Hz, 1H), 5.42-5.20 (m, 1H), 4.33-4.20 (m, 2H), 3.99-3.85 (m, 1H), 3.51-3.37 (m, 2H), 3.27 (d, J=3.6 Hz, 3H), 3.25-3.18 (m, 2H), 3.06-2.97 (m, 1H), 2.51-2.46 (m, 1H), 2.40 (d, J=2.4 Hz, 3H), 2.29-2.11 (m, 3H), 2.03-1.95 (m, 2H), 1.93-1.84 (m, 2H), 1.42-1.39 (m, 1H), 1.17-1.07 (m, 1H); LCMS (ESI, M+1): m/z=698.3.

### Example 239

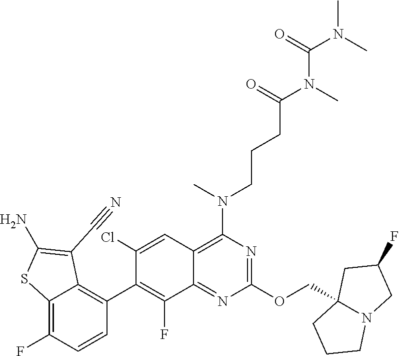 [SMILES: CN(C)C(=O)N(C)C(=O)CCCN(C)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(dimethylcarbamoyl)-N-methylbutanamide

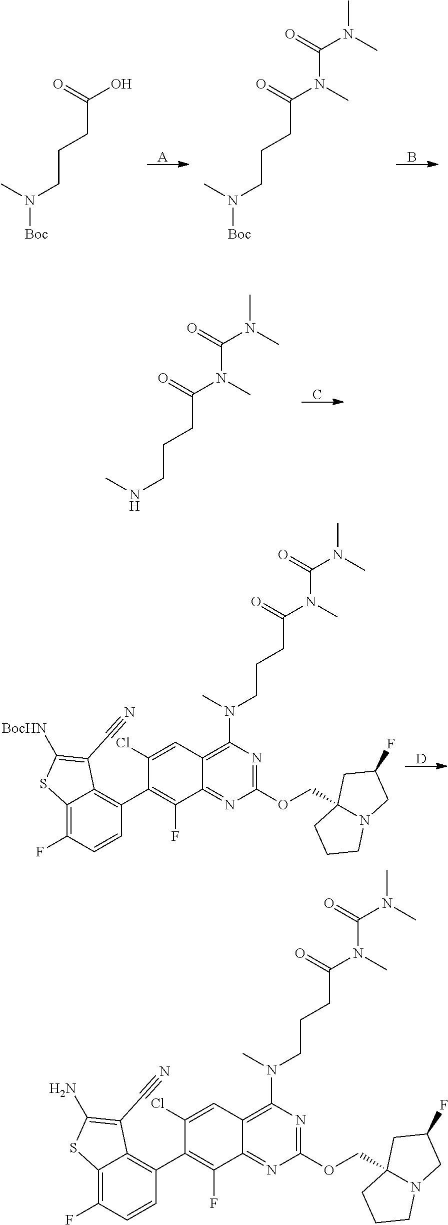

Step A. tert-butyl methyl(4-oxo-4-(1,3,3-trimethylureido)butyl)carbamate: To a solution of 4-((tert-butoxycarbonyl)(methyl)amino)butanoic acid (500 mg, 1.0 eq.) and 1,1,3-trimethylurea (705 mg, 3.0 eq.) in 2-methyltetrahydrofuran (5 mL) were added 2-chloro-1-methyl-pyridinium iodide (1.18 g, 2.0 eq.) and DIEA (892 mg, 3.0 eq.). The reaction was stirred at 80 °C for 1 hour. The reaction mixture was filtered, concentrated, diluted with water, and extracted with EtOAc (3×5 mL). The combined organic layers were washed with brine (2×10 mL), dried over anhydrous Na₂SO₄, filtered, concentrated and purified with reversed phase flash chromatography (0.1% FA condition) to afford the title compound (50.0 mg, 7.0% yield) as yellow oil; ¹H NMR (400 MHz, CHLOROFORM-d) δ=3.30-3.18 (m, 2H), 3.06-2.93 (m, 7H), 2.84 (s, 3H), 2.40-2.25 (m, 2H), 1.92-1.79 (m, 2H), 1.44 (s, 9H); LCMS (ESI, M+23): m/z=324.1.

Step B. N-(dimethylcarbamoyl)-N-methyl-4-(methylamino)butanamide: To a solution of tert-butyl methyl(4-oxo-4-(1,3,3-trimethylureido)butyl)carbamate (50.0 mg, 1.0 eq.) in DCM (0.6 mL) was added TFA (461 mg, 24.3 eq.) at 0 °C. The reaction was stirred at 25 °C for 1 hour. The mixture was concentrated, diluted with DMF (0.5 mL) and added DIEA to pH=7 to afford the title compound (30 mg, crude) as yellow oil; LCMS (ESI, M+1): m/z=202.1.

Step C. tert-butyl(4-(6-chloro-8-fluoro-2-(((2R,7aS)-2-fluorotetrahydro-1H-pyrrolizin-7a(5H)-yl)methoxy)-4-(methyl(4-oxo-4-(1,3,3-trimethylureido)butyl)amino)quinazolin-7-yl)-3-cyano-7-fluorobenzo[b]thiophen-2-yl)carbamate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 eq.) in DMF (0.7 mL) were added PyBOP (48.3 mg, 1.2 eq.) and DIEA (30.0 mg, 3.0 eq.). The reaction was stirred at 25 °C for 10 minutes, and then N-(dimethylcarbamoyl)-N-methyl-4-(methylamino)butanamide (18.7 mg, 1.2 eq.) was added dropwise. The reaction was stirred at 25 °C for 0.5 hour. The mixture was filtered and purified with prep-HPLC [Phenomenex luna C18 150×25 mm×10 μm; A: water (FA); B: ACN, B %: 28%-58% over 9 min] to afford the title compound (30.0 mg, 38% yield, HCOOH) as yellow solid; LCMS (ESI, M+1): m/z=829.4.

Step D. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(dimethylcarbamoyl)-N-methylbutanamide: To a solution of tert-butyl (4-(6-chloro-8-fluoro-2-(((2R,7aS)-2-fluorotetrahydro-1H-pyrrolizin-7a(5H)-yl)methoxy)-4-(methyl(4-oxo-4-(1,3,3-trimethylureido)butyl)amino)quinazolin-7-yl)-3-cyano-7-fluorobenzo[b]thiophen-2-yl)carbamate (30.0 mg, 1.0 equiv, HCOOH) in DCM (0.6 mL) were added TFA (0.3 mL, 118 eq.) at 0 °C. The reaction was stirred at 25 °C for 1 hour. The mixture was concentrated, diluted with MeOH (2 mL), neutralized with NaHCO₃, filtered and purified with prep-HPLC [Waters Xbridge 150×25 mm×5 μm; A: water (NH₄HCO₃); B: ACN, B %: 35%-65% over 10 min] to afford the title compound (6.35 mg, 25% yield) as off-white solid; ¹H NMR (400 MHz, METHANOL-d4) δ=8.09-8.06 (m, 1H), 7.20 (dd, J=5.6, 8.0 Hz, 1H), 7.03 (t, J=8.8 Hz, 1H), 5.38-5.21 (m, 1H), 4.30-4.18 (m, 2H), 3.95-3.83 (m, 2H), 3.51 (s, 3H), 3.25-3.18 (m, 2H), 3.17-3.11 (m, 1H), 3.02 (s, 3H), 2.99-2.91 (m, 6H), 2.53-2.45 (m, 2H), 2.31-2.23 (m, 1H), 2.16 (td, J=7.2, 14.0 Hz, 4H), 2.05-1.82 (m, 4H); LCMS (ESI, M+1): m/z=729.4.

### Example 240

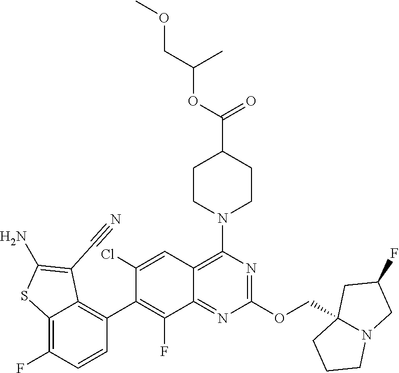 [SMILES: COCC(C)OC(=O)C1CCN(c2nc(OC[C@@]34CCCN3C[C@H](F)C4)nc3c(F)c(-c4ccc(F)c5sc(N)c(C#N)c45)c(Cl)cc23)CC1]

### 1-methoxypropan-2-yl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

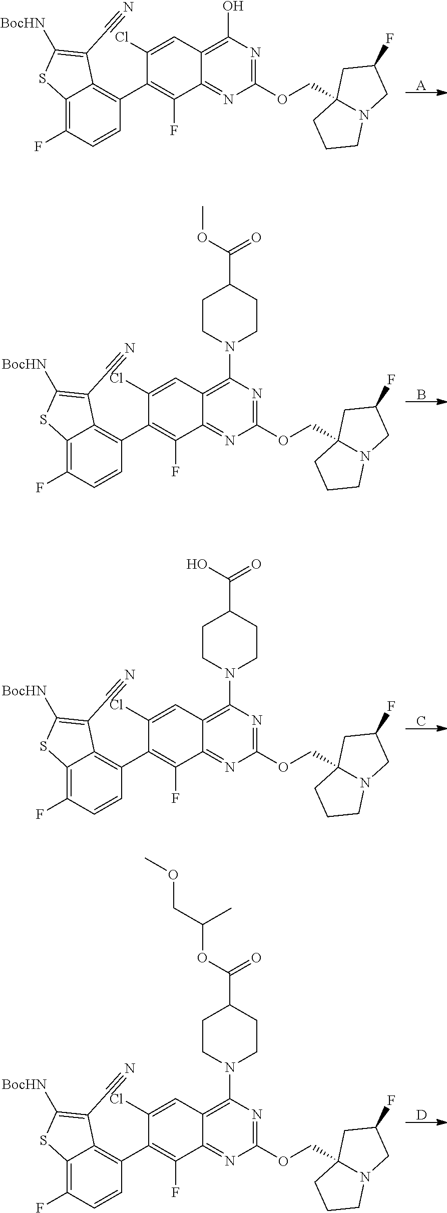

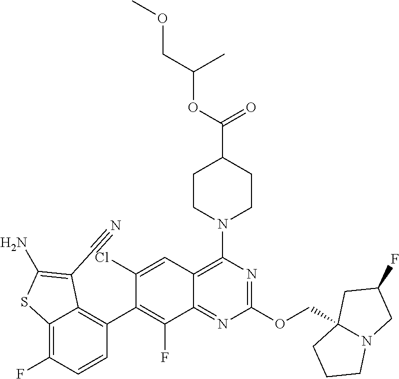 [SMILES: COCC(C)OC(=O)C1CCN(c2nc(OC[C@@]34CCCN3C[C@H](F)C4)nc3c(F)c(-c4ccc(F)c5sc(N)c(C#N)c45)c(Cl)cc23)CC1]

Step A. methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of tert-butyl(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (5.00 g, 1.0 eq.) and methyl piperidine-4-carboxylate (1.66 g, 1.5 eq.) in DMF (60 mL) were added DIEA (13.5 mL, 10 eq.), and PyBOP (6.00 g, 1.5 eq.). The reaction mixture was stirred at 15 °C for 1 hour. The mixture was diluted with water (100 mL) and extracted with ethyl acetate (100 mL). Then the organic phase was washed with water (3×50 mL), dried over anhydrous sodium sulfate, concentrated, and purified with column chromatography to afford the title compound (4.00 g, 66% yield) as yellow oil; ¹H NMR (400 MHz, DMSO-d₆) δ=11.31 (s, 1H), 7.85 (s, 1H), 7.39-7.03 (m, 2H), 5.53-5.22 (m, 1H), 4.43-4.12 (m, 4H), 3.65 (s, 3H), 3.50-3.34 (m, 5H), 3.11-2.94 (m, 1H), 2.86-2.73 (m, 1H), 2.33-2.09 (m, 3H), 2.07-2.01 (m, 2H), 1.99-1.69 (m, 5H), 1.46 (s, 9H); LCMS (ESI, M+1): m/z=771.3.

Step B. 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid: To a solution of methyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate (4.00 g, 1.0 eq.) in MeOH (50 mL) was added LiOH·H₂O (2.18 g, 10 eq.). The reaction mixture was stirred at 15 °C for 16 hours. The reaction mixture was concentrated under vacuum. Then it was diluted with water (50 mL) and extracted with ethyl acetate (50 mL). The organic phase was washed with water (30 mL). The pH of the aqueous phase was adjusted to 6 with HCl (2M), and then it was extracted with ethyl acetate (50 mL). The combined organic phase was washed with brine (30 mL), dried over anhydrous sodium sulfate, filtered and concentrated to afford the title compound (3.00 g, 75% yield) as yellow solid; ¹H NMR (400 MHz, DMSO-d₆) δ=12.1 (s, 1H), 7.79 (s, 1H), 7.19-6.86 (m, 2H), 5.43-5.16 (m, 1H), 4.30-4.10 (m, 4H), 3.44-3.32 (m, 5H), 2.93-2.82 (m, 1H), 2.70-2.58 (m, 1H), 2.23-2.07 (m, 3H), 2.01-1.96 (m, 2H), 1.88-1.67 (m, 5H), 1.41 (s, 9H); LCMS (ESI, M+1): m/z=757.2.

Step C. 1-methoxypropan-2-yl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (100 mg, 1.0 eq.) and 1-methoxypropan-2-ol (35.0 mg, 3.0 eq.) in DMF (1 mL) were added EDCI (38.0 mg, 1.5 eq.) and DMAP (24.0 mg, 1.5 eq.). The reaction mixture was stirred at 60 °C for 2 hours. The mixture was purified with HPLC to afford the title compound (60.0 mg, 52% yield) as yellow solid; ¹H NMR (400 MHz, DMSO-d₆) δ=11.8 (s, 1H), 7.96 (s, 1H), 7.58-7.39 (m, 2H), 5.75-5.50 (m, 1H), 5.05 (dt, J=4.4, 6.4 Hz, 1H), 4.72-4.54 (m, 2H), 4.44-4.23 (m, 2H), 3.94-3.70 (m, 3H), 3.56-3.41 (m, 5H), 3.31 (d, J=1.2 Hz, 4H), 2.88-2.77 (m, 1H), 2.40-2.34 (m, 1H), 2.29-2.04 (m, 6H), 1.93-1.78 (m, 2H), 1.56 (s, 9H), 1.20 (s, 3H); LCMS (ESI, M+1): m/z=829.2.

Step D. 1-methoxypropan-2-yl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: A solution of 1-methoxypropan-2-yl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate (60.0 mg, 1.0 eq.) in DCM (1 mL) was added TFA (1 mL). The reaction mixture was stirred at 15 °C for 0.5 hour. The reaction mixture was concentrated under vacuum. The residue was purified with HPLC. The desired fractions were collected and evaporated. The residue was redissolved in EtOAc (10 mL) and washed with saturated NaHCO₃ (3×15 mL). The organic phase was dried over anhydrous sodium sulfate, concentrated, and purified with HPLC to afford the title compound (11.85 mg, 22% yield) as white solid; ¹H NMR (400 MHz, METHANOL-d4) δ=7.84 (s, 1H), 7.21 (dd, J=5.2, 8.4 Hz, 1H), 7.04 (dd, J=8.4, 9.2 Hz, 1H), 5.42-5.20 (m, 1H), 5.16-5.05 (m, 1H), 4.46-4.34 (m, 2H), 4.34-4.21 (m, 2H), 3.51-3.41 (m, 4H), 3.36 (s, 3H), 3.30-3.18 (m, 3H), 3.03 (dt, J=5.2, 9.2 Hz, 1H), 2.84-2.72 (m, 1H), 2.42-2.20 (m, 2H), 2.19-2.07 (m, 3H), 2.05-1.86 (m, 5H), 1.23 (d, J=6.4 Hz, 3H); LCMS (ESI, M+1): m/z=729.3.

### Example 241

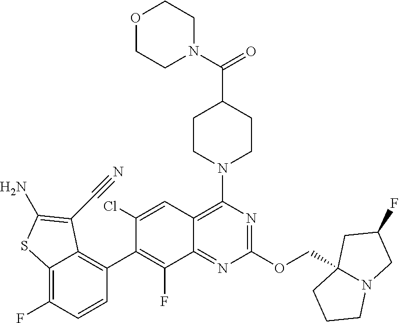 [SMILES: N#Cc1c(N)sc2c(F)ccc(-c3c(Cl)cc4c(N5CCC(C(=O)N6CCOCC6)CC5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(4-(morpholine-4-carbonyl)piperidin-1-yl)quinazolin-7-yl)-7-fluorobenzo [b]thiophene-3-carbonitrile

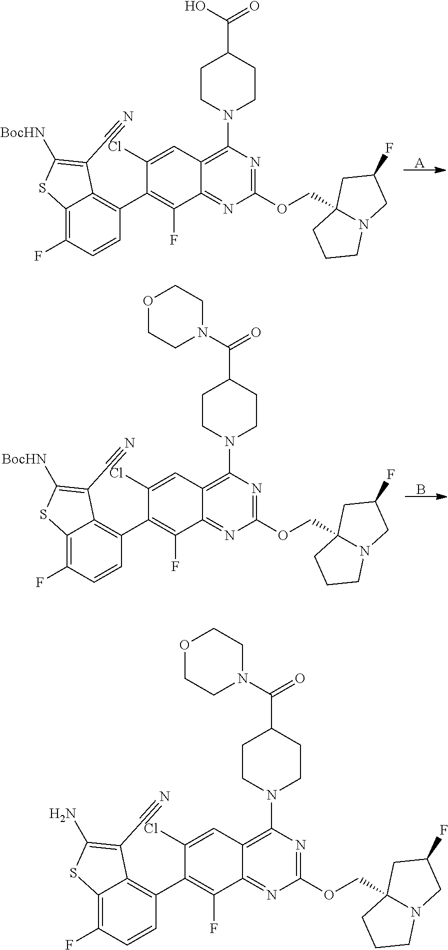

Step A. tert-butyl(4-(6-chloro-8-fluoro-2-(((2R,7aS)-2-fluorotetrahydro-1H-pyrrolizin-7a(5H)-yl)methoxy)-4-(4-(morpholine-4-carbonyl)piperidin-1-yl)quinazolin-7-yl)-3-cyano-7-fluorobenzo[b]thiophen-2-yl)carbamate: To a solution of 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (100 mg, 1.0 eq.) and morpholine (34.0 mg, 3.0 eq.) in DMF (1 mL) were added EDCI (38.0 mg, 1.5 eq.) and DMAP (24.0 mg, 1.5 eq.). The reaction mixture was stirred at 60 °C for 2 hours. The mixture was purified with HPLC to afford the title compound (50.0 mg, 45% yield) as yellow solid; ¹H NMR (400 MHz, DMSO-d₆) δ=11.33 (s, 1H), 7.86 (s, 1H), 7.41-7.03 (m, 2H), 5.54-5.25 (m, 1H), 4.44-4.16 (m, 4H), 3.70-3.52 (m, 6H), 3.51-3.34 (m, 7H), 3.13-2.92 (m, 2H), 2.36-2.09 (m, 3H), 2.05-1.72 (m, 7H), 1.46 (s, 9H); LCMS (ESI, M+1): m/z=826.2.

Step B. 2-amino-4-(6-chloro-8-fluoro-2-(((2R,7aS)-2-fluorotetrahydro-1H-pyrrolizin-7a(5H)-yl)methoxy)-4-(4-(morpholine-4-carbonyl)piperidin-1-yl)quinazolin-7-yl)-7-fluorobenzo[b]thiophene-3-carbonitrile: A solution of tert-butyl(4-(6-chloro-8-fluoro-2-(((2R,7aS)-2-fluorotetrahydro-1H-pyrrolizin-7a(5H)-yl)methoxy)-4-(4-(morpholine-4-carbonyl)piperidin-1-yl)quinazolin-7-yl)-3-cyano-7-fluorobenzo[b]thiophen-2-yl)carbamate (50.0 mg, 1.0 eq.) in DCM (1 mL) was added TFA (1 mL). The reaction mixture was stirred at 15 °C for 0.5 hour. The reaction mixture was concentrated under vacuum. The residue was purified with HPLC to afford the title compound that contained with TFA. The material was dissolved in EtOAc (10 mL) and washed with saturated NaHCO₃ (3×15 mL). The organic phase was dried over anhydrous sodium sulfate, filtered, concentrated and purified with HPLC to afford the title compound (9.60 mg, 20% yield) as white solid; ¹H NMR (400 MHz, METHANOL-d₄) δ=7.87 (d, J=1.6 Hz, 1H), 7.23 (dd, J=5.2, 8.4 Hz, 1H), 7.06 (dd, J=8.4, 9.2 Hz, 1H), 5.47-5.24 (m, 1H), 4.53 (d, J=13.6 Hz, 2H), 4.43-4.24 (m, 2H), 3.79-3.57 (m, 8H), 3.51-3.34 (m, 5H), 3.20-3.04 (m, 2H), 2.50-2.16 (m, 3H), 2.14-1.85 (m, 7H); LCMS (ESI, M+1): m/z=726.3.

### Example 242

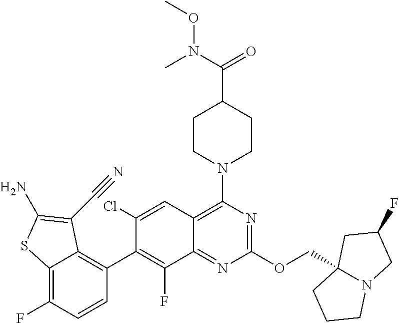 [SMILES: CON(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1]

### 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methoxy-N-methylpiperidine-4-carboxamide

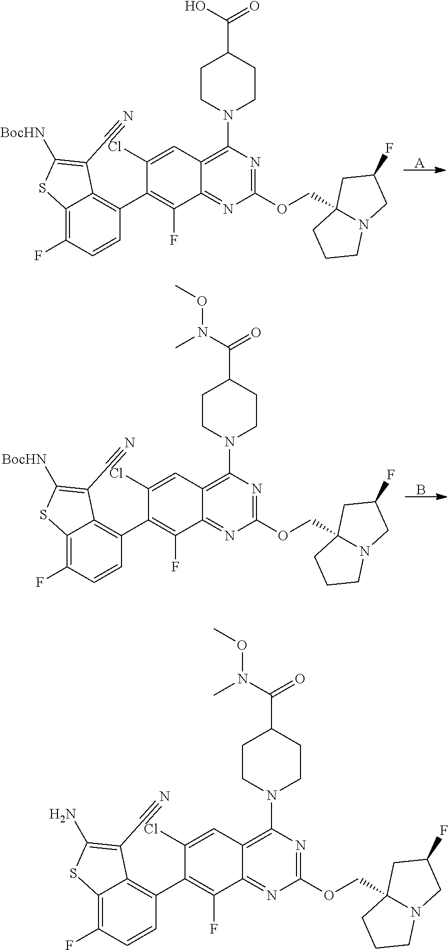

Step A. tert-butyl(4-(6-chloro-8-fluoro-2-(((2R,7aS)-2-fluorotetrahydro-1H-pyrrolizin-7a(5H)-yl)methoxy)-4-(4-(methoxy(methyl)carbamoyl)piperidin-1-yl)quinazolin-7-yl)-3-cyano-7-fluorobenzo[b]thiophen-2-yl)carbamate: To a solution of 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (100 mg, 1.0 eq.) and N-methoxymethanamine hydrochloride (25.0 mg, 1.9 eq.) in DMF (1 mL) were added EDCI (38.0 mg, 1.5 eq.) and DMAP (24.0 mg, 1.5 eq.). The reaction mixture was stirred at 60 °C for 1 hour. Then N-methoxymethanamine hydrochloride (25.0 mg, 1.9 eq.) was added at 20 °C The mixture was stirred at 60 °C for 1 h. The mixture was purified with HPLC to afford the title compound (35.0 mg, 26% yield) as yellow solid; ¹H NMR (400 MHz, DMSO-d₆) δ=11.4 (s, 1H), 7.84 (s, 1H), 7.30-7.07 (m, 2H), 5.48-5.24 (m, 1H), 4.40-4.14 (m, 4H), 3.74 (s, 3H), 3.50-3.39 (m, 2H), 3.17-2.89 (m, 6H), 2.32-1.65 (m, 12H), 1.45 (s, 9H); LCMS (ESI, M+1): m/z=800.2.

Step B. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methoxy-N-methylpiperidine-4-carboxamide: A solution of tert-butyl(4-(6-chloro-8-fluoro-2-(((2R,7aS)-2-fluorotetrahydro-1H-pyrrolizin-7a(5H)-yl)methoxy)-4-(4-(methoxy(methyl)carbamoyl)piperidin-1-yl)quinazolin-7-yl)-3-cyano-7-fluorobenzo[b]thiophen-2-yl)carbamate (35.0 mg, 1.0 eq.) in DCM (1 mL) was added TFA (1 mL). The reaction mixture was stirred at 20 °C for 0.5 hour. The mixture was diluted with water (15 mL) and neutralized with solid NaHCO₃. The mixture was extracted with DCM (3× 10 mL). The combined organic layer was washed with saturated NaHCO₃ aqueous solution (3×20 mL), dried over anhydrous sodium sulfate, concentrated and purified with HPLC to afford the title compound (13.62 mg, 51% yield) as white solid; ¹H NMR (400 MHz, DMSO-d₆) δ=8.11 (s, 2H), 7.82 (s, 1H), 7.27 (dd, J=5.2, 8.4 Hz, 1H), 7.20-7.08 (m, 1H), 5.37-5.18 (m, 1H), 4.40-4.25 (m, 2H), 4.15-4.06 (m, 1H), 4.05-3.98 (m, 1H), 3.73 (s, 3H), 3.47-3.28 (m, 5H), 3.11-3.03 (m, 3H), 3.01 (s, 1H), 2.87-2.77 (m, 1H), 2.20-2.10 (m, 1H), 2.09-1.97 (m, 2H), 1.90-1.68 (m, 7H); LCMS (ESI, M+1): m/z=700.2.

### Example 243

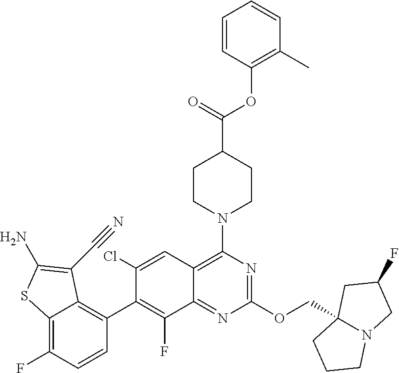 [SMILES: Cc1ccccc1OC(=O)C1CCN(c2nc(OC[C@@]34CCCN3C[C@H](F)C4)nc3c(F)c(-c4ccc(F)c5sc(N)c(C#N)c45)c(Cl)cc23)CC1]

### o-tolyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

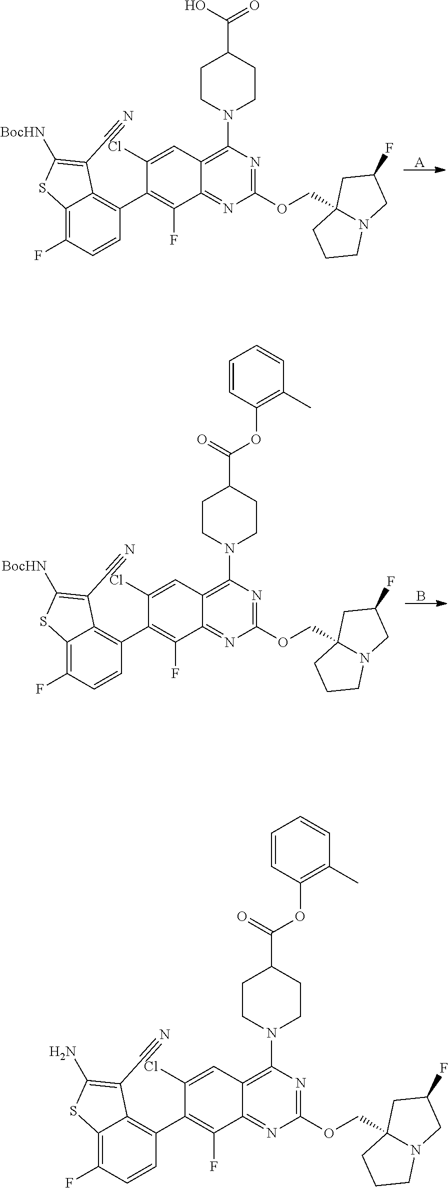

Step A. o-tolyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (100 mg, 1.0 eq.) and o-cresol (43.0 mg, 3.0 eq.) in DMF (1 mL) were added EDCI (38.0 mg, 1.5 eq.) and DMAP (24.0 mg, 1.5 eq.). The reaction mixture was stirred at 60 °C for 2 hours. The mixture was purified with HPLC to afford the title compound (60.0 mg, 50% yield) as yellow solid; ¹H NMR (400 MHz, DMSO-d₆) δ=11.3 (s, 1H), 7.92 (s, 1H), 7.37-7.18 (m, 6H), 5.53-5.29 (m, 1H), 4.46-4.24 (m, 4H), 3.60-3.38 (m, 5H), 3.20-2.98 (m, 2H), 2.35-2.21 (m, 5H), 2.13 (s, 3H), 2.07-1.87 (m, 5H), 1.47 (s, 9H); LCMS (ESI, M+1): m/z=847.2.

Step B. o-tolyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: A solution of o-tolyl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate (60.0 mg, 1.0 eq.) in DCM (1 mL) was added TFA (1 mL). The reaction mixture was stirred at 15 °C for 0.5 hour. The reaction mixture was concentrated under vacuum. The residue was purified with HPLC. The desired fractions were combined and concentrated. The residue was dissolved in EtOAc (10 mL) and washed with saturated NaHCO₃ (3×15 mL). The organic phase was dried over anhydrous sodium sulfate, and concentrated to afford the title compound (17.89 mg, 33% yield) as white solid; ¹H NMR (400 MHz, METHANOL-d₄) δ=7.88 (d, J=1.2 Hz, 1H), 7.32-7.11 (m, 4H), 7.08-6.98 (m, 2H), 5.44-5.21 (m, 1H), 4.55-4.41 (m, 2H), 4.39-4.21 (m, 2H), 3.62-3.44 (m, 2H), 3.43-3.33 (m, 1H), 3.26 (t, J=6.4 Hz, 2H), 3.17-2.99 (m, 2H), 2.45-2.21 (m, 4H), 2.18 (s, 3H), 2.17-1.84 (m, 6H); LCMS (ESI, M+1): m/z=747.3.

### Example 244

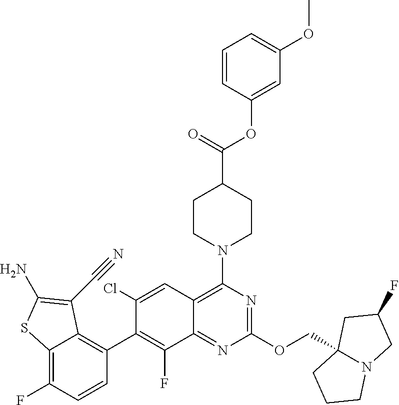 [SMILES: COc1cccc(OC(=O)C2CCN(c3nc(OC[C@@]45CCCN4C[C@H](F)C5)nc4c(F)c(-c5ccc(F)c6sc(N)c(C#N)c56)c(Cl)cc34)CC2)c1]

### 3-methoxyphenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 243. The title compound was obtained as white solid; ¹H NMR (400 MHz, METHANOL-d₄) δ=7.88 (s, 1H), 7.29 (t, J=8.4 Hz, 1H), 7.21 (dd, J=5.2, 8.4 Hz, 1H), 7.04 (t, J=8.8 Hz, 1H), 6.88-6.79 (m, 1H), 6.73-6.65 (m, 2H), 5.46-5.23 (m, 1H), 4.55-4.41 (m, 2H), 4.40-4.25 (m, 2H), 3.80 (s, 3H), 3.60-3.47 (m, 2H), 3.46-3.33 (m, 2H), 3.26 (m, 1H), 3.12-3.00 (m, 2H), 2.49-2.15 (m, 5H), 2.13-1.90 (m, 5H); LCMS (ESI, M+1): m/z=763.3.

### Example 245

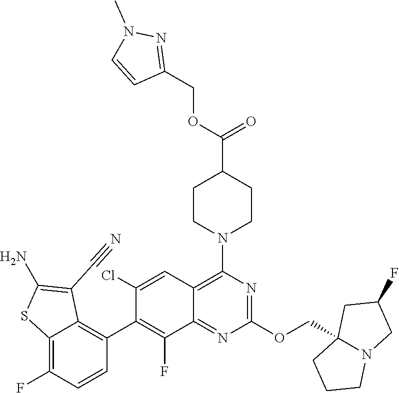 [SMILES: Cn1ccc(COC(=O)C2CCN(c3nc(OC[C@@]45CCCN4C[C@H](F)C5)nc4c(F)c(-c5ccc(F)c6sc(N)c(C#N)c56)c(Cl)cc34)CC2)n1]

### (1-methyl-1H-pyrazol-3-yl)methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 243. The title compound was obtained as white solid; ¹H NMR (400 MHz, METHANOL-d₄) δ=7.83 (s, 1H), 7.55 (d, J=2.4 Hz, 1H), 7.20 (dd, J=5.2, 8.4 Hz, 1H), 7.03 (dd, J=8.4, 9.2 Hz, 1H), 6.31 (d, J=2.4 Hz, 1H), 5.42-5.22 (m, 1H), 5.12 (s, 2H), 4.43-4.19 (m, 4H), 3.87 (s, 3H), 3.53-3.38 (m, 2H), 3.30-3.20 (m, 3H), 3.03 (dt, J=5.6, 9.2 Hz, 1H), 2.87-2.74 (m, 1H), 2.43-2.05 (m, 5H), 2.05-1.80 (m, 5H); LCMS (ESI, M+1): m/z=751.3.

### Example 246

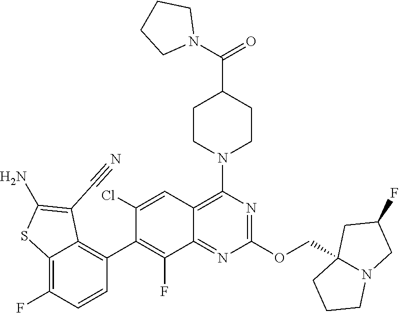 [SMILES: N#Cc1c(N)sc2c(F)ccc(-c3c(Cl)cc4c(N5CCC(C(=O)N6CCCC6)CC5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(4-(pyrrolidine-1-carbonyl)piperidin-1-yl)quinazolin-7-yl)-7-fluorobenzo [b]thiophene-3-carbonitrile

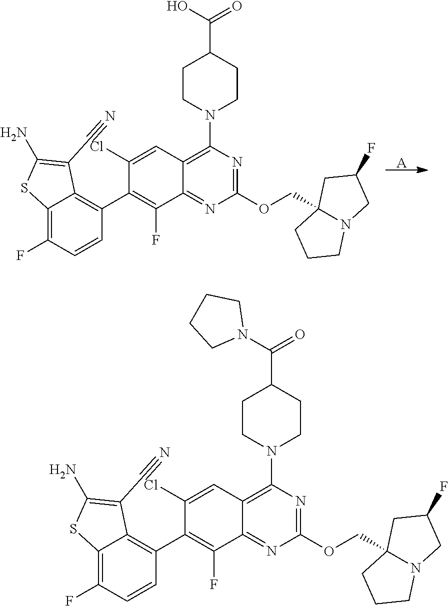

Step A. 2-amino-4-(6-chloro-8-fluoro-2-(((2R,7aS)-2-fluorotetrahydro-1H-pyrrolizin-7a(5H)-yl)methoxy)-4-(4-(pyrrolidine-1-carbonyl)piperidin-1-yl)quinazolin-7-yl)-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (50 mg, 1.0 eq.) and pyrrolidine (27.1 mg, 5.0 eq.) in DMF (0.5 mL) were added DIEA (49.2 mg, 5.0 eq.) and HATU (57.9 mg, 2.0 eq.). The reaction was stirred at 25 °C for 0.5 hour. The mixture was filtered and the filtrate was purified with prep-HPLC [Waters Xbridge C18 150×25 mm×5 μm; A: water (ammonia hydroxide); B: ACN, B %: 40%-70% over 11 min] to afford the title compound (9.63 mg, 17.5% yield) as yellow solid; ¹H NMR (400 MHz, CHLOROFORM-d₃) δ=7.74 (d, J=1.2 Hz, 1H), 7.22 (dd, J=4.8, 8.0 Hz, 1H), 7.04 (t, J=8.6 Hz, 1H), 5.45-5.36 (m, 2H), 5.34 (br s, 1H), 4.50-4.37 (m, 2H), 4.32-4.22 (m, 1H), 4.20-4.11 (m, 1H), 3.53 (m, 4H), 3.31-3.13 (m, 5H), 3.02-2.91 (m, 1H), 2.78-2.67 (m, 1H), 2.29 (br s, 1H), 2.24-2.08 (m, 4H), 2.05-1.99 (m, 2H), 1.97-1.84 (m, 7H); LCMS (ESI, M+1): m/z=710.3.

### Example 247

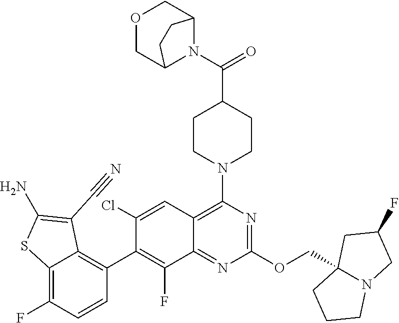 [SMILES: N#Cc1c(N)sc2c(F)ccc(-c3c(Cl)cc4c(N5CCC(C(=O)N6C7CCC6COC7)CC5)nc(OC[C@@]56CCCN5C[C@H](F)C6)nc4c3F)c12]

### 4-(4-(4-(3-oxa-8-azabicyclo[3.2.1]octane-8-carbonyl)piperidin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

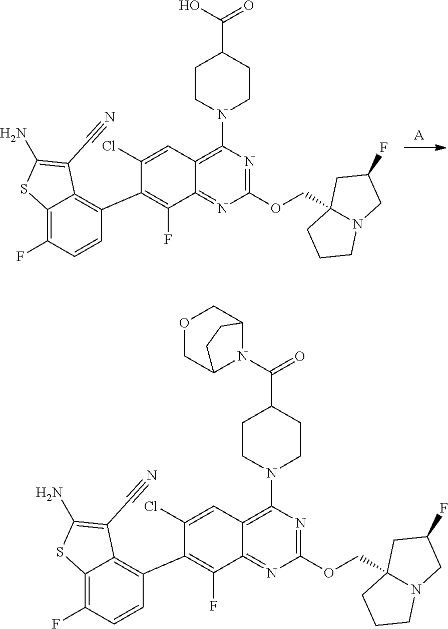

Step A. 4-(4-(4-(3-oxa-8-azabicyclo[3.2.1]octane-8-carbonyl)piperidin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (50.0 mg, 1.0 eq.) in DMF (1 mL) were added DIEA (49.2 mg, 5.0 eq.), (1R,5S)-3-oxa-8-azabicyclo[3.2.1]octane hydrochloride (17.1 mg, 1.5 eq.) and HATU (43.4 mg, 1.5 eq.). The reaction was stirred at 25 °C for 0.5 hour. The mixture was filtered and purified with prep-HPLC [column: Waters Xbridge 150×25 mm×5 μm; mobile phase: [water (NH₄HCO₃)-ACN]; gradient: 38%-68% B over 10 min] to afford the title compound (14.4 mg, 25% yield) as yellow solid; ¹H NMR (400 MHz, METHANOL-d₄) δ=7.85 (s, 1H), 7.21 (dd, J=5.2, 8.4 Hz, 1H), 7.04 (t, J=8.8 Hz, 1H), 5.37-5.23 (m, 1H), 4.57-4.46 (m, 3H), 4.43 (br d, J=6.0 Hz, 1H), 4.31-4.20 (m, 2H), 3.71-3.65 (m, 4H), 3.43-3.36 (m, 2H), 3.23-3.13 (m, 3H), 3.06-2.97 (m, 2H), 2.38-2.20 (m, 2H), 2.16-1.95 (m, 9H), 1.90-1.87 (m, 3H); LCMS (ESI, M+1): m/z=752.3.

### Example 248

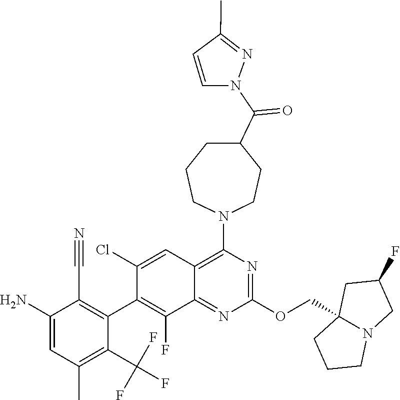 [SMILES: Cc1ccn(C(=O)C2CCCN(c3nc(OC[C@@]45CCCN4C[C@H](F)C5)nc4c(F)c(-c5c(C#N)c(N)cc(C)c5C(F)(F)F)c(Cl)cc34)CC2)n1]

### 6-amino-2-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl)azepan-1-yl)quinazolin-7-yl)-4-methyl-3-(trifluoromethyl)benzonitrile

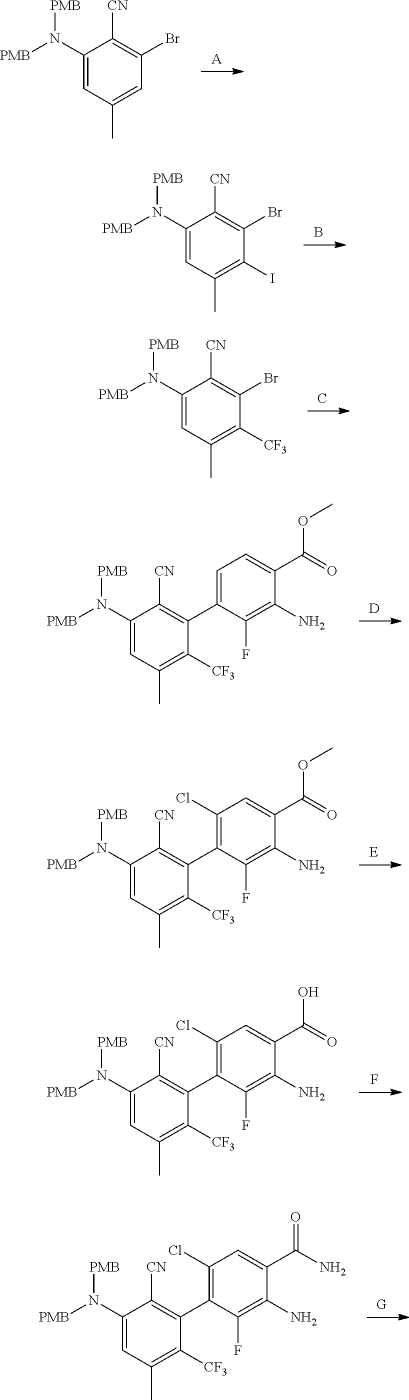

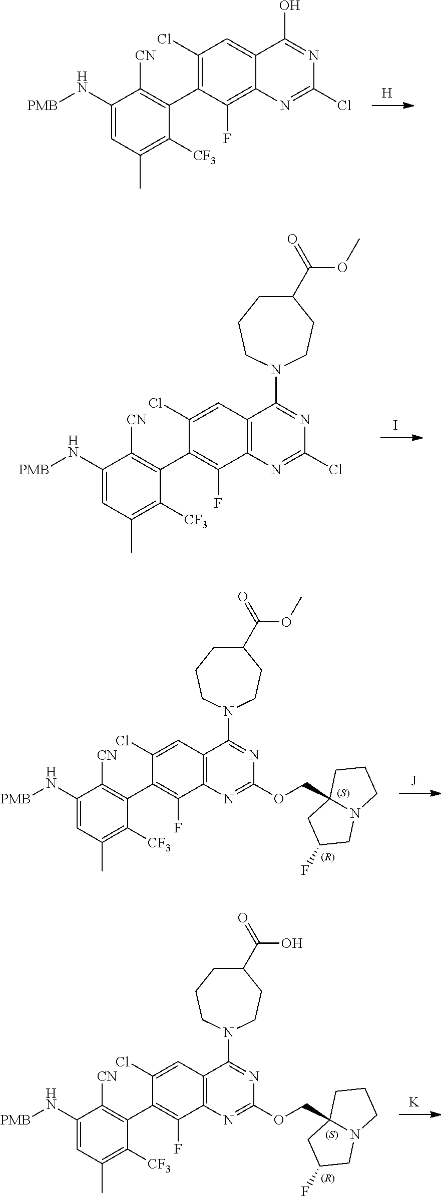

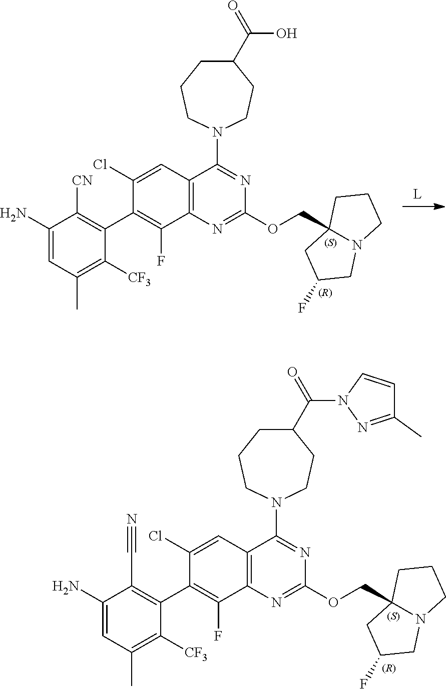

Step A: 6-(bis(4-methoxybenzyl)amino)-2-bromo-3-iodo-4-methylbenzonitrile: A mixture of 2-(bis(4-methoxybenzyl)amino)-6-bromo-4-methylbenzonitrile (40.7 g, 90.2 mmol, 1.00 eq.), NIS (50.7 g, 225 mmol, 2.50 eq.) in AcOH (200 mL) was degassed and purged with N₂ for 3 times. The reaction was stirred at 20 °C for 0.5 hr under N₂ atmosphere. The reaction was quenched with 15.0% Na₂S₂O₃.aqueous (500 mL) and NaOH aqueous (200 mL) solution, and the mixture was extracted with ethyl acetate 100 mL twice. The combined organic layers were washed with brine 100 mL, dried over anhydrous Na₂SO₄, filtered, and concentrated under reduced pressure. The residue was purified with prep-HPLC (column: Phenomenex luna C18 250 mm×100 mm×10 μm; mobile phase: [water (TFA)-ACN]; gradient: 65%-95% B over 20 mins. The title compound was obtained as a yellow oil (28.4 g, 48.9 mmol, 54.3% yield, 99.5% purity).

Step B: 6-(bis(4-methoxybenzyl)amino)-2-bromo-4-methyl-3-(trifluoromethyl)benzonitrile: A mixture of 6-(bis(4-methoxybenzyl)amino)-2-bromo-3-iodo-4-methylbenzonitrile (28.4 g, 49.2 mmol, 1.00 eq.), methyl 2,2-difluoro-2-(fluorosulfonyl)acetate (28.4 g, 148 mmol, 18.8 mL, 3.00 eq), CuI (23.4 g, 123 mmol, 2.50 eq.) in DMF (280 mL) was degassed and purged with N₂ for 3 time. The mixture was stirred at 90 °C for 8 hours under N₂ atmosphere. The reaction mixture was quenched with H₂O 400 mL at 25 °C, and then extracted with ethyl acetate 200 mL twice. The combined organic layers were washed with brine, dried over anhydrous Na₂SO₄, filtered, and concentrated. The residue was purified with column chromatography (SiO₂, petroleum ether/ethyl acetate=20/1 to 10/1). The title compound was obtained as a yellow solid (20.6 g, 38.4 mmol, 78.1% yield, 96.9% purity).

Step C. methyl 3-amino-3′-(bis(4-methoxybenzyl)amino)-2′-cyano-2-fluoro-5′-methyl-6′-(trifluoromethyl)-[1,1′-biphenyl]-4-carboxylate: To a mixture of methyl 2-amino-3-fluoro-4-(4,4,5,5-tetramethyl-1,3,2-dioxaborolan-2-yl)benzoate (6.40 g, 1.00 eq.), 6-(bis(4-methoxybenzyl)amino)-2-bromo-4-methyl-3-(trifluoromethyl)benzonitrile (11.26 g, eq.), K₃PO₄ (2 M, 21.7 mL, eq.), Pd(PPh₃)₄ (2.51 g, 0.10 eq.) in dioxane (100 mL) was degassed and purged with N₂ for 3 times, and then the reaction was stirred at 90 °C for 3 hours under N₂ atmosphere. The mixture was diluted with water (100 mL) and extracted with EtOAc (2×50 mL). The combined organic layers were washed with brine (100 mL), dried over anhydrous Na₂SO₄, filtered, and concentrated under reduced pressure to give a residue. The residue was purified by column chromatography (SiO₂, Petroleum ether/Ethyl acetate=20/1 to 10/1, Petroleum ether/Ethyl acetate=5/1, R_{f}=0.15) to afford the title compound (7.60 g, 47% yield) as yellow solid; ¹H NMR (400 MHz, CDCl₃) δ=7.73 (dd, J=1.2 Hz, J=8.4 Hz, 1H), 7.14-7.11 (m, 4H), 6.88-6.84 (m, 5H), 6.48 (dd, J=6.8 Hz, J=8.4 Hz, 1H), 5.91 (s, 2H), 4.45 (d, J=0.8 Hz, 4H), 3.91 (s, 3H), 3.81 (s, 6H); LCMS (ESI, M+1); LCMS (ESI, M+1): m/z=608.2.

Step D. methyl 3-amino-3′-(bis(4-methoxybenzyl)amino)-6-chloro-2′-cyano-2-fluoro-5′-methyl-6′-(trifluoromethyl)-[1,1′-biphenyl]-4-carboxylate: To a solution of methyl 3-amino-3′-(bis(4-methoxybenzyl)amino)-2′-cyano-2-fluoro-5′-methyl-6′-(trifluoromethyl)-[1,1′-biphenyl]-4-carboxylate (6.40 g, 1.00 eq.) and 6-(bis(4-methoxybenzyl)amino)-2-bromo-4-methyl-3-(trifluoromethyl)benzonitrile (6.20 g, 1.00 equiv.) in DMF (60.0 mL) was added NCS (1.64 g, 1.20 eq.). The reaction was degassed and purged with N₂ for 3 times, and then was stirred at 90 °C for 1 hour under N₂ atmosphere. The mixture was diluted with water (100 mL) and extracted with EtOAc (2×100 mL). The combined organic layers were washed with brine (100 mL), dried over anhydrous Na₂SO₄, filtered, and concentrated under reduced pressure to give a residue. The residue was purified by column chromatography (SiO₂, Petroleum ether/Ethyl acetate=20/1 to 10/1, Petroleum ether/Ethyl acetate=4/1, R_{f}=0.31) to afford the title compound (4.20 g, 48.3% yield) as yellow solid; ¹H NMR (400 MHz, CDCl₃) δ=7.83 (d, J=1.6 Hz, 1H), 7.12 (d, J=8.4 Hz, 4H), 6.87-6.83 (m, 5H), 5.89 (s, 2H), 4.53-4.41 (m, 4H), 3.93 (s, 3H), 3.81 (s, 6H), 2.48 (d, J=2.4 Hz, 2H); LCMS (ESI, M+1): m/z=642.0.

Step E. 3-amino-3′-(bis(4-methoxybenzyl)amino)-6-chloro-2′-cyano-2-fluoro-5′-methyl-6′-(trifluoromethyl)-[1,1′-biphenyl]-4-carboxylic acid: A solution of methyl 3-amino-3′-(bis(4-methoxybenzyl)amino)-6-chloro-2′-cyano-2-fluoro-5′-methyl-6′-(trifluoromethyl)-[1,1′-biphenyl]-4-carboxylate (4.00 g, 1.00 eq.) and LiOH·H₂O (523 mg, 2.00 eq.) in ACN (50.0 mL) and H₂O (10.0 mL) was degassed and purged with N₂ for 3 times. The reaction was stirred at 20 °C for 24 hours under N₂ atmosphere. The mixture was diluted with water (10.0 mL) and acidified with HCl (1 M, 2.00 mL). The solution was extracted with EtOAc (2×20.0 mL). The combined organic layers were washed with brine (20.0 mL), dried over anhydrous Na₂SO₄, filtered and concentrated under reduced pressure to give a residue. The residue was purified by prep-HPLC (column: Waters Xbridge BEH C18 250*50 mm*10 μm; mobile phase: [water (NH₄HCO₃)-ACN]; gradient: 15%-45% B over 20 min) to afford the title compound (3.40 g, 85.0% yield,) as yellow solid; ¹H NMR (400 MHz, CDCl₃)₈-7.90 (s, 1H), 7.07 (d, J=7.6 Hz, 4H), 6.81-6.76 (m, 5H), 5.75 (s, 1H), 4.40 (q, J=15.2 Hz, 4H), 3.74 (s, 6H), 2.41 (s, 3H); LCMS (ESI, M+1): m/z=628.1.

Step F 3-amino-3′-(bis(4-methoxybenzyl)amino)-6-chloro-2′-cyano-2-fluoro-5′-methyl-6′-(trifluoromethyl)-[1,1′-biphenyl]-4-carboxamide: To a solution of 3-amino-3′-(bis(4-methoxybenzyl)amino)-6-chloro-2′-cyano-2-fluoro-5′-methyl-6′-(trifluoromethyl)-[1,1′-biphenyl]-4-carboxylic acid (2.40 g, 1.00 eq.) and DIEA (1.48 g, 2.00 mL, 3.00 eq.) in DMF (20.0 mL) were added NH₄Cl (613 mg, 3.00 eq.) and HATU (2.18 g, 1.50 eq.). The reaction was degassed and purged with N₂ for 3 times, and then was stirred at 20 °C for 0.5 hour under N₂ atmosphere. The mixture was diluted 20 mL water and filtered. The filtrate was concentrated under reduced pressure and purified by column chromatography (SiO₂, Petroleum ether/Ethyl acetate=20/1 to 0/1, Petroleum ether/Ethyl acetate=1/1, R_{f}=0.20) to afford the title compound (3.40 g, 85% yield) as yellow solid; ¹H NMR (400 MHz, CDCl₃) δ=7.35 (d, J=1.6 Hz, 1H), 7.12 (d, J=8.4 Hz, 4H), 6.86 (d, J=8.8 Hz, 5H), 5.75 (s, 2H), 4.47 (q, J=14.8 Hz, 4H), 3.81 (s, 6H), 2.48 (d, J=2.0 Hz, 3H); LCMS (ESI, M+1): m/z=627.1.

Step G 2-(2,6-dichloro-8-fluoro-4-hydroxyquinazolin-7-yl)-6-((4-methoxybenzyl)amino)-4-methyl-3-(trifluoromethyl)benzonitrile: To a solution of 3-amino-3′-(bis(4-methoxybenzyl)amino)-6-chloro-2′-cyano-2-fluoro-5′-methyl-6′-(trifluoromethyl)-[1,1′-biphenyl]-4-carboxamide (2.00 g, 1.00 eq.) in dioxane (20.0 mL) was added thiophosgene (367 mg, 245 μL, 1.00 eq.). The reaction was degassed and purged with N₂ for 3 times, and then stirred at 80 °C for 0.5 hour under N₂ atmosphere. The mixture was diluted with water (10.0 mL) and extracted with EtOAc (5.00 mL). The combined organic layers were washed with brine (100 mL), dried over anhydrous Na₂SO₄, filtered and concentrated under reduced pressure to give a residue. The residue was purified by column chromatography (SiO₂, Petroleum ether/Ethyl acetate=20/1 to 2/1, Petroleum ether/Ethyl acetate=2/1, R_{f}=0.27) to afford the title compound (2.40 g, 75% yield) as yellow solid; LCMS (ESI, M+1): m/z=551.0.

Step H methyl 1-(2,6-dichloro-7-(2-cyano-3-((4-methoxybenzyl)amino)-5-methyl-6-(trifluoromethyl)phenyl)-8-fluoroquinazolin-4-yl)azepane-4-carboxylate: To a solution of 2-(2,6-dichloro-8-fluoro-4-hydroxyquinazolin-7-yl)-6-((4-methoxybenzyl)amino)-4-methyl-3-(trifluoromethyl)benzonitrile (1.00 g, 1.00 eq.) and methyl azepane-4-carboxylate (422 mg, 1.20 equiv, HCl) in DCM (10.0 mL) were added BOP-C1 (1.39 g, 3.00 eq.) and DIPEA (2.34 g, 10.0 eq.). The reaction was degassed and purged with N₂ for 3 times, and then stirred at 20 °C for 12 hours under N₂ atmosphere. The mixture was diluted with water (20.0 mL) and extracted with EtOAc. The combined organic layers were washed with brine (10.0 mL), dried over anhydrous Na₂SO₄, filtered, and concentrated under reduced pressure to give a residue. The residue was purified by column chromatography (SiO₂, Petroleum ether/Ethyl acetate=20/1 to 2/1, Petroleum ether/Ethyl acetate=2/1, R_{f}=0.27) to afford the title compound (1.00 g, 78% yield) as yellow solid; LCMS (ESI, M+1): m/z=690.1.

Step I methyl 1-(6-chloro-7-(2-cyano-3-((4-methoxybenzyl)amino)-5-methyl-6-(trifluoromethyl)phenyl)-8-fluoro-2-(((2R,7aS)-2-fluorotetrahydro-1H-pyrrolizin-7a(5H)-yl)methoxy)quinazolin-4-yl)azepane-4-carboxylate: To a solution of methyl 1-(2,6-dichloro-7-(2-cyano-3-((4-methoxybenzyl)amino)-5-methyl-6-(trifluoromethyl)phenyl)-8-fluoroquinazolin-4-yl)azepane-4-carboxylate (400 mg, 1.00 eq.) and ((2R,7aS)-2-fluorotetrahydro-1H-pyrrolizin-7a(5H)-yl)methanol (184 mg, 2.00 eq.) in dioxane (4.00 mL) were added Pd₂(dba)₃ (53.1 mg, 0.10 eq.), BINAP (72.1 mg, 0.20 eq.) and t-BuONa (111 mg, 2.00 eq.). The reaction was degassed and purged with N₂ for 3 times. The reaction was then stirred at 80 °C for 12 hours under N₂ atmosphere. The mixture was filtered and concentrated under reduced pressure to afford the title compound (400 mg, crude) as brown oil; LCMS (ESI, M+1): m/z=813.3.

Step J 1-(6-chloro-7-(2-cyano-3-((4-methoxybenzyl)amino)-5-methyl-6-(trifluoromethyl)phenyl)-8-fluoro-2-(((2R,7aS)-2-fluorotetrahydro-1H-pyrrolizin-7a(5H)-yl)methoxy)quinazolin-4-yl)azepane-4-carboxylic acid: A solution of methyl 1-(6-chloro-7-(2-cyano-3-((4-methoxybenzyl)amino)-5-methyl-6-(trifluoromethyl)phenyl)-8-fluoro-2-(((2R,7aS)-2-fluorotetrahydro-1H-pyrrolizin-7a(5H)-yl)methoxy)quinazolin-4-yl)azepane-4-carboxylate (400 mg, 41.00 eq.) and LiOH·H₂O (103 mg, 5.00 eq.) in ACN (4.00 mL) and H₂O (4.00 mL) was degassed and purged with N₂ for 3 times, and then stirred at 20 °C for 12 hours under N₂ atmosphere. The pH of the mixture was adjusted to 3~4 with sat. HCl (1M), and then extracted with DCM (2×10.0 mL). The combined organic layer was dried over with Na₂SO₄, concentrated and purified by column chromatography (SiO₂, DCM/MeOH=100/0 to 10/1, DCM/MeOH=10/1, R_{f}=0.30) pressure to afford the title compound (300 mg, 258 μmol, 52.4% yield) as yellow solid; LCMS (ESI, M+1): m/z=799.2.

Step K 1-(7-(3-amino-2-cyano-5-methyl-6-(trifluoromethyl)phenyl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a solution of 1-(6-chloro-7-(2-cyano-3-((4-methoxybenzyl)amino)-5-methyl-6-(trifluoromethyl)phenyl)-8-fluoro-2-(((2R,7aS)-2-fluorotetrahydro-1H-pyrrolizin-7a(5H)-yl)methoxy)quinazolin-4-yl)azepane-4-carboxylic acid (100 mg, 1.00 eq.) in DCM (3.00 mL) was added TFA (285 mg, 186 μL, 20.0 eq.). The reaction was stirred at 20 °C for 2 hours. The mixture was concentrated under reduced pressure to afford the title compound (100 mg, crude) as yellow oil; LCMS (ESI, M+1): m/z=679.1.

Step L 6-amino-2-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl)azepan-1-yl)quinazolin-7-yl)-4-methyl-3-(trifluoromethyl)benzonitrile: To a solution of 1-(7-(3-amino-2-cyano-5-methyl-6-(trifluoromethyl)phenyl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (100 mg, 1.00 eq.), 3-methyl-1H-pyrazole (24.2 mg, 2.00 eq.) and DIEA (95.2 mg, 128 μL, 5.00 eq.) in DCM (2.00 mL) was added HATU (84.0 mg, 1.50 eq.). The reaction was degassed and purged with N₂ for 3 times, and then stirred at 20 °C for 1 hour under N₂ atmosphere. The mixture was diluted with water (5.00 mL) and extracted with DCM (3×5 mL). The combined organic layers were washed with brine (10.0 mL), dried over anhydrous Na₂SO₄, filtered, and concentrated under reduced pressure to give a residue. The residue was purified by prep-HPLC (column: Phenomenex luna C18 150*25 mm*10 μm; mobile phase: [water (FA)-ACN]; gradient: 22%-52% B over 15 min) pressure to afford the title compound (6.00 mg, 5.4% yield) as white solid; ¹H NMR (400 MHz, CDCl₃) δ=8.14 (d, J=2.0 Hz, 1H), 7.98 (s, 1H), 6.86 (s, 1H), 6.28 (s, 1H), 5.70-5.50 (m, 1H), 5.00-4.90 (m, 1H), 4.85-4.80 (m, 1H), 4.62-4.50 (m, 1H), 4.29-4.28 (s, 1H), 4.10-3.90 (m, 4H), 3.85-3.75 (m, 1H), 3.70-3.60 (m, 1H), 3.55-3.31 (m, 1H), 2.63 (s, 3H), 2.49-2.40 (m, 2H), 2.32-2.29 (m, 7H), 2.05-1.90 (m, 2H), 1.32-1.36 (m, 4H); ¹⁹F NMR (400 MHz, CDCl₃) δ=−55.39, −71.88,−73.77; LCMS (ESI, M+1): m/z=743.3.

### Example 249

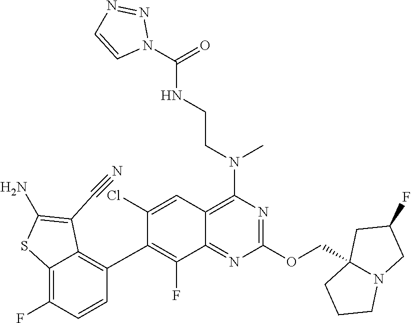 [SMILES: CN(CCNC(=O)n1ccnn1)c1nc(OC[C@@]23CCCN2C[C@H](F)C3)nc2c(F)c(-c3ccc(F)c4sc(N)c(C#N)c34)c(Cl)cc12]

### N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-1H-1,2,3-triazole-1-carboxamide

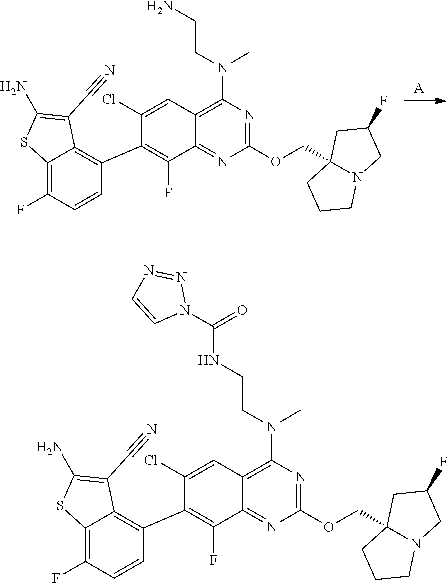

Step A. N-(2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)ethyl)-2H-1,2,3-triazole-2-carboxamide: To a solution of 1H-1,2,3-triazole (20.0 mg, 1.0 eq.) and DIEA (150 mg, 4.0 eq.) in dichloromethane (1 mL) was added bis(trichloromethyl) carbonate (40.0 mg, 0.47 eq.) at 0 °C. The reaction was stirred at 0 °C for 1 hour. A solution of 2-amino-4-(4-((2-aminoethyl)(methyl)amino)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (157 mg, 0.90 eq.) in THF (5 mL) was added dropwise at 0 °C. The reaction was stirred at 25 °C for 15 hours. The mixture was quenched with saturated NaHCO₃ aqueous solution (3 mL) and extracted with dichloromethane (3×2 mL). The combined organic layers were washed with brine (5 mL), dried over anhydrous sodium sulfate and concentrated. The crude was purified with prep-HPLC [Waters Xbridge 150×25 mm×5 μm; A: water (NH₄HCO₃); B %: ACN, B %: 40%-70% over 9 min], prep-chiral SFC [DAICEL CHIRALPAK AD (250 mm×30 mm, 10 μm); mobile phase: [CO₂-i-PrOH (0.1% NH₃·H₂O)]; B %: 40%, isocratic elution mode] and prep-HPLC [Phenomenex luna C18 150×25 mm×10 μm; A: water (FA); B %: ACN, B %: 18%-48% over 9 min] to afford the title compound (5.24 mg, 2.6% yield) as off-white solid; t_{R}=0.681 min; ¹H NMR (400 MHz, METHANOL-d4) δ=8.58-8.48 (m, 1H), 8.35-7.95 (m, 1H), 7.78 (br s, 1H), 7.43 (br s, 1H), 7.20 (dd, J=5.2, 8.4 Hz, 1H), 7.09-6.94 (m, 1H), 5.64 (d, J=5.2 Hz, 1H), 4.71-4.61 (m, 1H), 4.60-4.44 (m, 2H), 4.43-4.32 (m, 1H), 4.19-3.98 (m, 1H), 3.98-3.80 (m, 3H), 3.79-3.58 (m, 4H), 3.55-3.41 (m, 1H), 2.70-2.51 (m, 1H), 2.51-2.41 (m, 1H), 2.41-2.32 (m, 1H), 2.30-2.19 (m, 2H), 2.16-2.06 (m, 1H); LCMS (ESI, M+1): m/z=697.3.

### Example 250

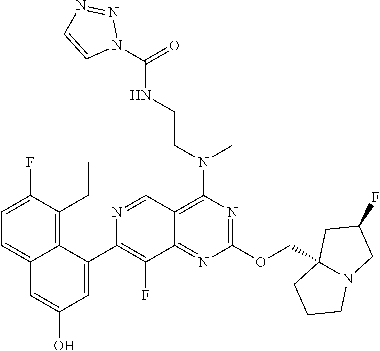 [SMILES: CCc1c(F)ccc2cc(O)cc(-c3ncc4c(N(C)CCNC(=O)n5ccnn5)nc(OC[C@@]56CCCN5C[C@H](F)C6)nc4c3F)c12]

### N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-1H-1,2,3-triazole-1-carboxamide

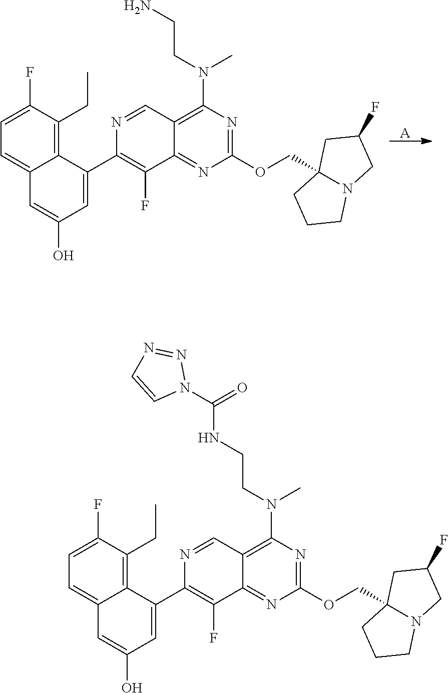

Step A. N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-1H-1,2,3-triazole-1-carboxamide: To a solution of 1H-1,2,3-triazole (18.0 mg, 1.0 eq.) and DIEA (135 mg, 4.0 eq.) in dichloromethane (1 mL) was added bis(trichloromethyl) carbonate (50.0 mg, 0.65 eq.) at 0 °C. The reaction was stirred at 0 °C for 1 hour. 4-(4-((2-aminoethyl)(methyl)amino)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol (133 mg, 0.9 eq.) in THF (5 mL) was added at 0 °C. The reaction was stirred at 25 °C for 15 hours. The mixture was quenched with saturated NaHCO₃ aqueous solution (3 mL) and extracted with dichloromethane (3×2 mL). The combined organic layers were washed with brine (5 mL), dried over anhydrous sodium sulfate, filtered, and concentrated. The residue was purified with prep-HPLC [Waters Xbridge 150×25 mm×5 μm; A: water (NH₄HCO₃); B: ACN, B %: 32%-62% over 9 min], prep-chiral SFC [DAICEL CHIRALPAK AD (250 mm×30 mm, 10 μm); mobile phase: [CO₂-i-PrOH (0.1% NH3·H2O)]; B %: 40%, isocratic elution mode] and prep-HPLC [Phenomenex luna C18 150×25 mm×10 μm; A: water (FA); B %: ACN, B %: 18%-48% over 9 min] to afford the title compound (20.0 mg, 2.6% yield) white solid; ¹H NMR (400 MHz, METHANOL-d4) δ=9.45-8.97 (m, 1H), 8.61-8.46 (m, 1H), 8.38-8.03 (m, 1H), 7.87-7.58 (m, 2H), 7.31 (d, J=2.8 Hz, 1H), 7.25 (t, J=9.2 Hz, 1H), 7.11-6.99 (m, 1H), 5.61-5.27 (m, 1H), 4.61-4.36 (m, 3H), 4.19-3.88 (m, 4H), 3.41 (br s, 4H), 3.26-3.13 (m, 1H), 2.62-2.33 (m, 3H), 2.30-1.94 (m, 6H), 0.86-0.68 (m, 3H); LCMS (ESI, M+1): m/z=662.5.

### Example 251

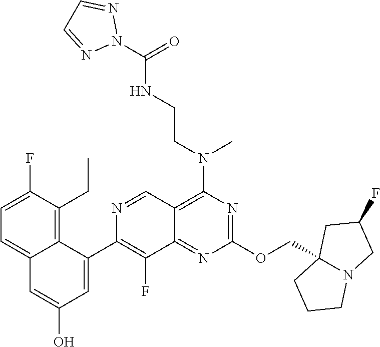 [SMILES: CCc1c(F)ccc2cc(O)cc(-c3ncc4c(N(C)CCNC(=O)n5nccn5)nc(OC[C@@]56CCCN5C[C@H](F)C6)nc4c3F)c12]

### N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-2H-1,2,3-triazole-2-carboxamide

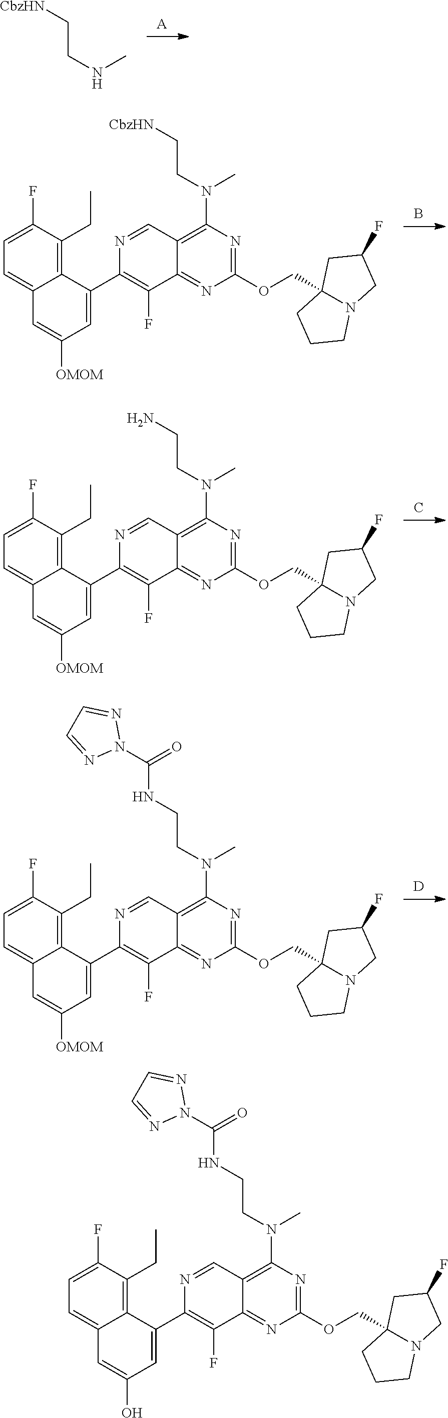

Step A. benzyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)carbamate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (900 mg, 1.0 eq.) in DMF (10 mL) were added PyBOP (1.01 g, 1.2 eq.) and DIEA (629 mg, 3.0 eq.). The reaction was stirred at 25 °C for 20 minutes. Benzyl(2-(methylamino)ethyl)carbamate (405 mg, 1.2 eq.) was added. The reaction was stirred at 25 °C for 40 minutes. The mixture was filtered and purified with reversed phase flash chromatography (0.1% NH₃·H₂O) to afford the title compound (1.00 g, 79% yield over two steps) as a yellow solid; LCMS (ESI, M+1): m/z=745.4.

Step B. N-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylethane-1,2-diamine: To a mixture of Pd/C (200 mg, 0.16 eq.) in MeOH (20 mL) was added benzyl(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)carbamate (900 mg, 1.0 eq.) under N₂. The suspension was degassed under vacuum and purged with H₂ several times. The mixture was stirred under H₂ (15 psi) at 25 °C for 3 hours. The crude product was purified with reversed phase flash chromatography (0.1% FA condition) to afford the title compound (700 mg, 95% yield) as yellow oil; LCMS (ESI, M+1): m/z=611.4

Step C: N-(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-2H-1,2,3-triazole-2-carboxamide: To a solution of 1H-1,2,3-triazole (7.00 mg, 1.0 eq.) and DIEA (52.4 mg, 4.0 eq.) in dichloromethane (0.5 mL) was added bis(trichloromethyl) carbonate (10.0 mg, 0.33 eq.) at 0 °C. The reaction was stirred at 0 °C for 1 hour. N1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N1-methylethane-1,2-diamine (51.7 mg, 0.9 eq.) and DIEA (19.6 mg, 1.5 eq.) in dichloromethane (0.5 mL) were added dropwise at 0 °C and the reaction was stirred at 25 °C for 15 hours. The mixture was quenched with saturated NaHCO₃ aqueous solution (3 mL) and extracted with dichloromethane (3×2 mL). The combined organic layers were washed with brine (5 mL), dried over anhydrous sodium sulfate and filtered. The mixture was concentrated and purified by revered-phase flash (0.1% FA condition) and prep-chiral HPLC [DAICEL CHIRALCEL OD (250 mm×30 mm, 10 μm); A: [CO2-ACN/i-PrOH (0.1% NH₃H₂O)]; B: B %: 55%, isocratic elution mode] to afford the title compound (80.0 mg, 42% yield) as yellow solid. t_{R}=1.682 min; ¹H NMR (400 MHz, METHANOL-d4) δ=9.21-8.93 (m, 1H), 8.03-7.85 (m, 1H), 7.78 (br dd, J=6.0, 9.2 Hz, 1H), 7.73 (br dd, J=3.2, 5.6 Hz, 1H), 7.64-7.59 (m, 1H), 7.30 (t, J=9.6 Hz, 1H), 7.21 (br s, 1H), 5.30-5.14 (m, 1H), 4.36-4.20 (m, 2H), 4.15-3.85 (m, 6H), 3.50 (s, 3H), 3.39-3.31 (m, 2H), 3.28-3.15 (m, 4H), 3.01 (br d, J=7.6 Hz, 1H), 2.54-2.28 (m, 2H), 2.27-2.08 (m, 4H), 2.03-1.87 (m, 3H), 1.15 (d, J=6.0 Hz, 3H), 0.86-0.72 (m, 3H); LCMS (ESI, M+1): m/z=706.5.

Step D: N-(2-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-2H-1,2,3-triazole-2-carboxamide: To a solution of N-(2-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)ethyl)-2H-1,2,3-triazole-2-carboxamide (40.0 mg, 1.0 eq.) in dichloromethane (2 mL) was added TFA (1 mL, 237 eq.) at 0 °C. The reaction was stirred at 25 °C for 0.5 hour. The mixture was concentrated, redissolved in DMF (2 mL) and water (2 mL), basified with NaHCO₃solid and filtered. The filtrate was concentrated and purified with prep-HPLC [Phenomenex luna C18 150×25 mm×10 μm; A: water (FA); B: ACN, B %: 12%-42% B over 9 min] to afford the title compound (20.0 mg, 52% yield) as yellow solid. ¹H NMR (400 MHz, METHANOL-d4) δ=9.41-9.00 (m, 1H), 7.95-7.71 (m, 1H), 7.71-7.64 (m, 1H), 7.31 (d, J=2.8 Hz, 1H), 7.28-7.20 (m, 1H), 7.11-7.02 (m, 1H), 5.42-5.16 (m, 1H), 4.31-4.19 (m, 2H), 4.15-4.06 (m, 2H), 4.05-3.84 (m, 1H), 3.70 (t, J=7.6 Hz, 2H), 3.38-3.32 (m, 1H), 3.28-3.15 (m, 3H), 3.01 (s, 3H), 2.54-2.40 (m, 1H), 2.39-2.27 (m, 1H), 2.26-2.08 (m, 3H), 2.07-2.02 (m, 1H), 2.00-1.96 (m, 1H), 1.95-1.83 (m, 1H), 0.79 (t, J=7.6 Hz, 3H); LCMS (ESI, M+1): m/z=662.4

### Example 252

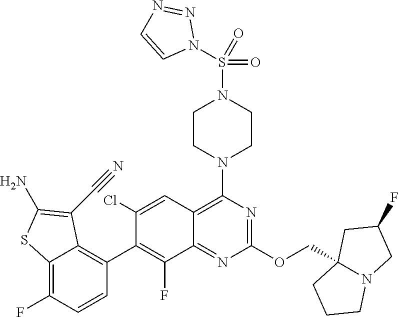 [SMILES: N#Cc1c(N)sc2c(F)ccc(-c3c(Cl)cc4c(N5CCN(S(=O)(=O)n6ccnn6)CC5)nc(OC[C@@]56CCCN5C[C@H](F)C6)nc4c3F)c12]

### 4-(4-(4-((1H-1,2,3-triazol-1-yl) sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

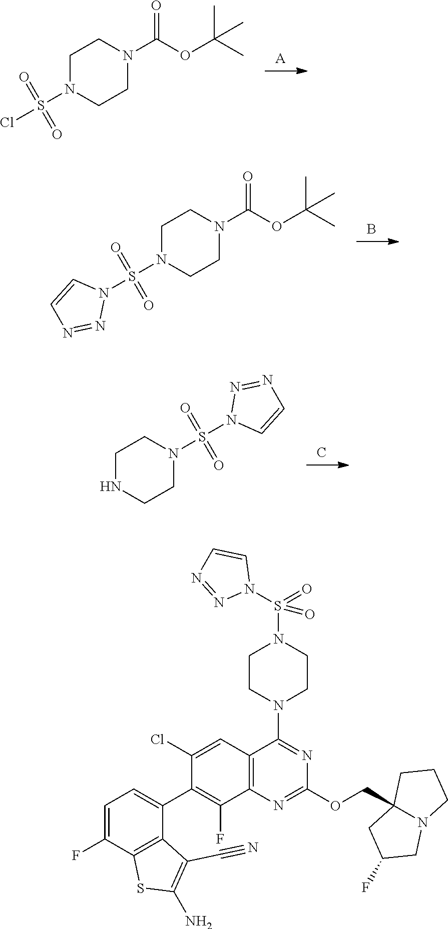

Step A. tert-butyl 4-((1H-1,2,3-triazol-1-yl) sulfonyl) piperazine-1-carboxylate: To a solution of tert-butyl 4-(chlorosulfonyl) piperazine-1-carboxylate (1.00 g, 1.0 eq.) in ethyl alcohol (10 mL) were added 1H-1,2,3-triazole (242 mg, 2.0 eq.) and potassium carbonate (1.50 g, 3.0 eq.) at 25 °C The reaction was stirred at 25 °C for 1 hour. The mixture was filtered. The filtrate was purified with prep-HPLC [Phenomenex luna C18 150×40 mm×15 μm; A: water (0.1% FA), B: ACN, B %: 35%-65% over 15 min] to afford the title compound (400 mg, 36% yield) as white solid; ¹H NMR (400 MHz, METHANOL-d₄) δ=8.44 (d, J=1.2 Hz, 1H), 7.87 (d, J=0.8 Hz, 1H), 3.52 (br d, J=4.8 Hz, 4H), 3.43-3.34 (m, 4H), 1.44 (s, 9H)

Step B. 1-(triazol-1-ylsulfonyl) piperazine: To a solution of tert-butyl 4-(triazol-1-ylsulfonyl) piperazine-1-carboxylate (110 mg, 1.0 eq.) in dichloromethane (0.5 mL) was added trifluoroacetic acid (0.5 mL) at 0 °C. The reaction was stirred at 0 °C for 1 hour. The mixture was concentrated to afford the title compound (100 mg, 87% yield, TFA) as yellow oil; ¹H NMR (400 MHz, METHANOL-d₄) δ=8.51 (d, J=1.2 Hz, 1H), 7.91 (d, J=1.2 Hz, 1H), 3.75-3.66 (m, 4H), 3.41-3.36 (m, 4H).

Step C. 4-(4-(4-((1H-1,2,3-triazol-1-yl) sulfonyl) piperazin-1-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (80.0 mg, 1.0 eq.) and 1-(triazol-1-ylsulfonyl) piperazine (97.1 mg, 2.0 equiv, TFA) in dimethylformamide (1 mL) were added PyBOP (107 mg, 1.4 eq.) and DIEA (228 mg, 12.0 eq.) at 25 °C. The reaction was stirred at 25 °C for 2 hours. The mixture was filtered and purified with prep-HPLC [Waters Xbridge 150×25 mm×5 μm; A: water (10 mM NH₄HCO₃), B: ACN, B %: 42%-72% over 9 min] and further purified with prep-HPLC [Phenomenex luna C18 150×25 mm×10 μm; A: water (0.1% FA), B: ACN, B %: 20%-50% over 9 min] to afford the title compound (8.03 mg, 7.3% yield, 0.21 HCOOH) as yellow solid; ¹H NMR (400 MHz, METHANOL-d₄) δ=8.49 (d, J=0.8 Hz, 1H), 7.89 (d, J=1.2 Hz, 1H), 7.88 (d, J=1.2 Hz, 1H), 7.21 (dd, J=5.2, 8.4 Hz, 1H), 7.05 (t, J=9.2 Hz, 1H), 5.50-5.22 (m, 1H), 4.40-4.25 (m, 2H), 4.08-3.95 (m, 4H), 3.68 (br t, J=4.8 Hz, 4H), 3.50-3.34 (m, 3H), 3.15-3.06 (m, 1H), 2.46-2.26 (m, 2H), 2.24-2.15 (m, 1H), 2.11-2.01 (m, 2H), 2.00-1.87 (m, 1H); LCMS (ESI, M+1): m/z=745.2.

### Example 253

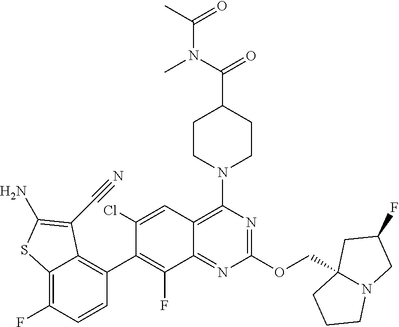 [SMILES: CC(=O)N(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1]

### N-acetyl-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylpiperidine -4-carboxamide

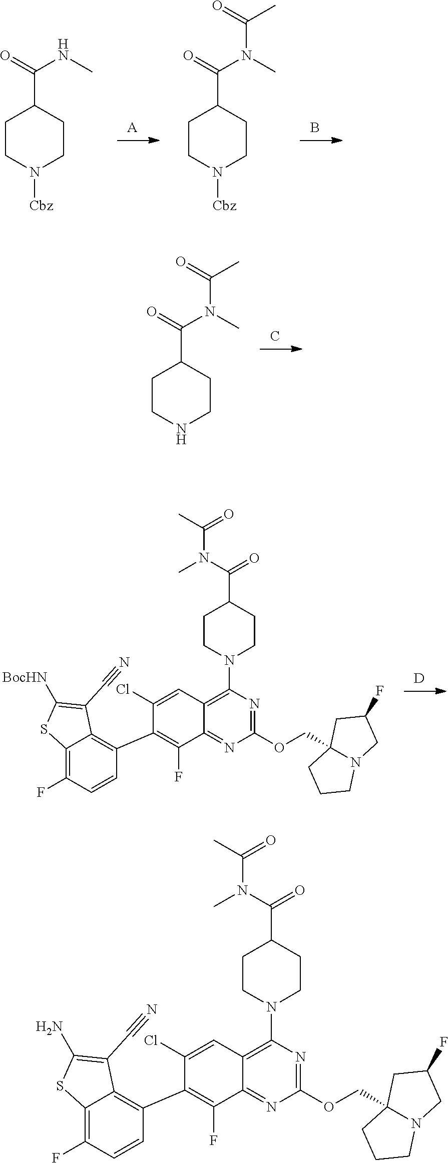

Step A. benzyl 4-(acetyl(methyl)carbamoyl)piperidine-1-carboxylate: To a solution of b benzyl 4-(methylcarbamoyl)piperidine-1-carboxylate (200 mg, 1.0 eq.) and acetyl chloride (170 mg, 3.0 eq.) in 2-MeTHF (2.0 mL) was added DIEA (468 mg, 5.0 eq.). The reaction was stirred at 80 °C for 4 hours. The mixture was diluted with water (2 mL) and extracted with ethyl acetate (3 mL). The organic layer was dried over anhydrous sodium sulfate, concentrated, and purified with reversed phase flash chromatography (0.1% formic acid condition) to afford the title compound (340 mg, crude) as yellow solid; LCMS (ESI, M+1): m/z=319.1.

Step B. N-acetyl-N-methylpiperidine-4-carboxamide: To a suspension of Pd/C (668 mg, 1.0 eq.) in EtOAc (2.0 mL) was added benzyl 4-(acetyl(methyl)carbamoyl)piperidine-1-carboxylate (200 mg, 1.0 eq.) under nitrogen. The reaction was degassed and purged with H₂ 3 times. The reaction was stirred at 30 °C for 2 hours under H₂ atmosphere (15 Psi). The mixture was filtered and concentrated to afford the title compound (120 mg, crude) as yellow solid.

Step C. tert-butyl (4-(4-(4-(acetyl(methyl)carbamoyl)piperidin-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 eq.) and N-acetyl-N-methylpiperidine-4-carboxamide (25.7 mg, 1.8 eq.) in DMF (0.5 mL) were added PyBOP (32.2 mg, 0.8 eq.) and DIEA (30.0 mg, 3.0 eq.). The reaction was stirred at 30 °C for 2 hours. The mixture was diluted with water (2 mL) and extracted with ethyl acetate (3 mL). The organic layer was dried over anhydrous sodium sulfate, filtered, and concentrated to afford the title compound (40 mg, crude) as yellow oil; LCMS (ESI, M+1): m/z=812.3.

Step D. N-acetyl-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylpiperidine-4-carboxamide: To a solution of tert-butyl (4-(4-(4-(acetyl(methyl)carbamoyl)piperidin-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (30.0 mg, 1.0 eq.) in DCM (0.5 mL) was added TFA (767 mg, 182 eq.). The reaction was stirred at 25 °C for 2 hours. The mixture was diluted with water (2 mL) and extracted with ethyl acetate (3×2 mL). The combined organic layer was dried over anhydrous sodium sulfate, concentrated, and purified with prep-HPLC [Phenomenex luna C18 150×25 mm×10 μm; A: water (FA); B: ACN, B %: 25%-55% over 9 min] to afford the title compound (4.21 mg, 15% yield) as yellow solid; ¹H NMR (400 MHz, DIMETHYL SULFOXIDE-d₆) δ=8.17-8.07 (m, 2H), 7.85 (s, 1H), 7.31-7.22 (m, 1H), 7.19-7.12 (m, 1H), 5.54-5.18 (m, 1H), 4.33 (br s, 2H), 4.26-4.00 (m, 2H), 3.57-3.46 (m, 1H), 3.44-3.33 (m, 2H), 3.30-3.20 (m, 2H), 3.18 (s, 3H), 3.16-3.08 (m, 1H), 3.00-2.83 (m, 1H), 2.34 (s, 3H), 2.26-2.20 (m, 1H), 2.15-2.10 (m, 1H), 1.98 (br d, J=11.2 Hz, 3H), 1.91-1.67 (m, 5H); LCMS (ESI, M+1): m/z=712.4.

### Example 254

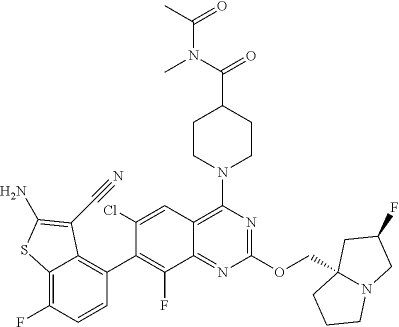 [SMILES: CC(=O)N(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1]

### N-acetyl-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylpiperidine-4-carboxamide

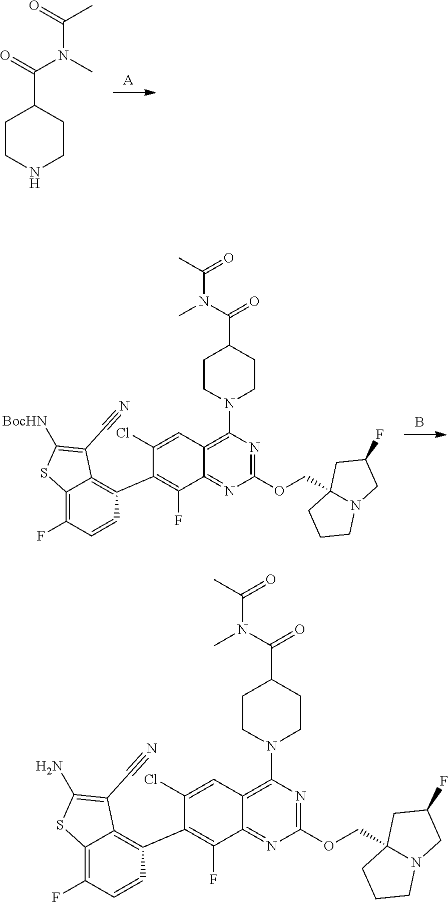

Step A. tert-butyl (4-((S)-4-(4-(acetyl(methyl)carbamoyl)piperidin-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 eq.) and N-acetyl-N-methylpiperidine-4-carboxamide (25.7 mg, 1.8 eq.) in DMF (0.5 mL) were added PyBOP (32.2 mg, 0.8 eq.) and DIEA (30.0 mg, 3.0 eq.). The reaction was stirred at 30 °C for 2 hours. The mixture was diluted with water (2 mL) and extracted with ethyl acetate (3 mL). The organic layer was dried over anhydrous sodium sulfate, filtered, and concentrated to afford the title compound (40 mg, crude) as yellow oil; LCMS (ESI, M+1): m/z=812.2.

Step B. N-acetyl-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylpiperidine-4-carboxamide: To a solution of tert-butyl (4-((S)-4-(4-(acetyl(methyl)carbamoyl)piperidin-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (30.0 mg, 1.0 eq.) in DCM (0.5 mL) was added TFA (767 mg, 182 eq.). The reaction was stirred at 25 °C for 2 hours. The mixture was diluted with water (2 mL) and extracted with ethyl acetate (3×2 mL). The organic layer was dried over anhydrous sodium sulfate, concentrated and purified with prep-HPLC [Waters Xbridge 150×25 mm×5 μm; A: water (NH₄HCO₃); B: ACN, B %: 45%-75% over 10 min] to afford the title compound (4.53 mg, 6% yield) as off-white solid; ¹H NMR (400 MHz, DIMETHYL SULFOXIDE-d₆) δ=8.12 (s, 2H), 7.84 (s, 1H), 7.27 (dd, J=5.2, 8.4 Hz, 1H), 7.18-7.12 (m, 1H), 5.39-5.14 (m, 1H), 4.31 (br t, J=14.4 Hz, 2H), 4.14-3.95 (m, 2H), 3.57-3.44 (m, 1H), 3.42-3.35 (m, 2H), 3.18 (s, 3H), 3.12-2.97 (m, 3H), 2.87-2.76 (m, 1H), 2.34 (s, 3H), 2.19-2.10 (m, 1H), 2.09-1.92 (m, 4H), 1.88-1.68 (m, 5H); LCMS (ESI, M+1): m/z=712.3.

### Example 255

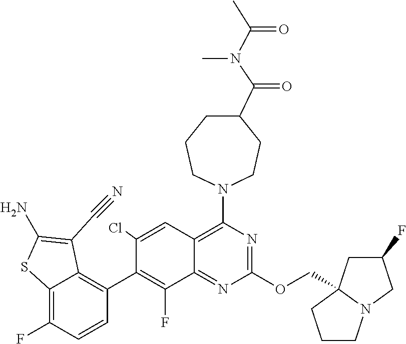 [SMILES: CC(=O)N(C)C(=O)C1CCCN(c2nc(OC[C@@]34CCCN3C[C@H](F)C4)nc3c(F)c(-c4ccc(F)c5sc(N)c(C#N)c45)c(Cl)cc23)CC1]

### N-acetyl-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylazepane-4-carboxamide

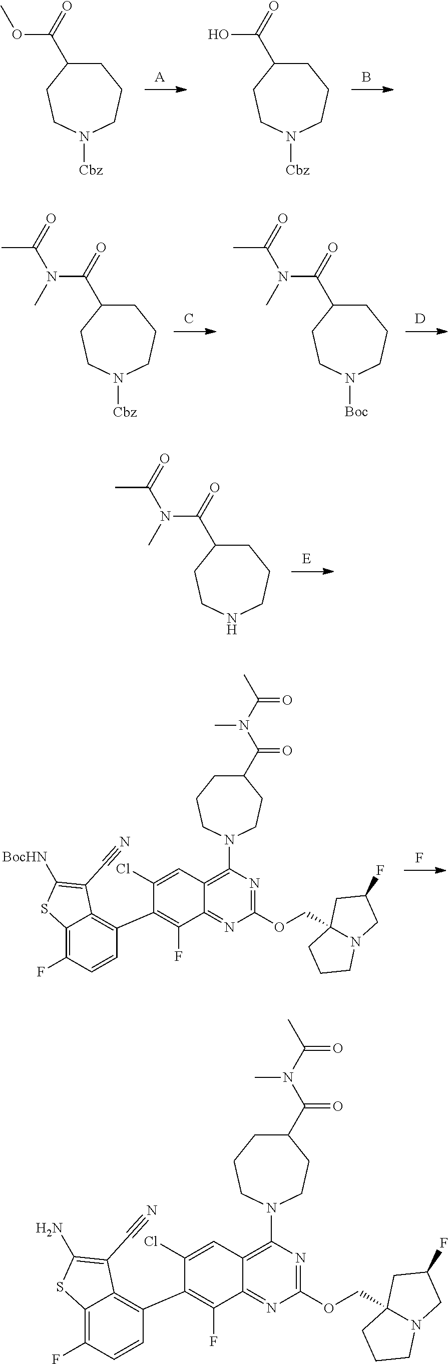

Step A. 1-((benzyloxy)carbonyl)azepane-4-carboxylic acid: To a mixture of 1-benzyl 4-methyl azepane-1,4-dicarboxylate (3.50 g, 1.0 eq.) in THF (9 mL), H₂O (9 mL) and MeOH (6 mL) was added LiOH·H₂O (1.51 g, 3.0 eq.). The reaction was stirred at 20 °C for 2 hours. The mixture was filtered and purified with reversed phase flash chromatography [C18, 0.1% formic acid condition] to afford the title compound (3.3 g, 97% yield) as yellow liquid; LCMS (ESI, M+1): m/z=278.0.

Step B. benzyl 4-(acetyl(methyl)carbamoyl)azepane-1-carboxylate: To a mixture of 1-((benzyloxy)carbonyl)azepane-4-carboxylic acid (1.65 g, 1.0 eq.), N-methylacetamide (870 mg, 2.0 eq.) and DIEA (3.84 g, 5.0 eq.) in 2-methyltetrahydrofuran (30 mL) was added CMPI (4.56 g, 3.0 eq.). The reaction was stirred at 60 °C for 2 hours. The mixture was concentrated and purified with reversed phase flash chromatography [C18, 0.1% formic acid condition] to afford the title compound (800 mg, 19% yield) as yellow solid; ¹H NMR (400 MHz, DMSO-d₆) δ=7.46-7.24 (m, 5H), 5.08 (s, 2H), 3.59-3.46 (m, 2H), 3.41-3.36 (m, 1H), 3.32-3.24 (m, 1H), 3.23-3.04 (m, 4H), 2.29 (s, 3H), 1.98-1.76 (m, 3H), 1.73-1.48 (m, 2H), 1.47-1.33 (m, 1H); LCMS (ESI, M+23): m/z=355.1.

Step C. tert-butyl 4-(acetyl(methyl)carbamoyl)azepane-1-carboxylate: To a mixture of 1-((benzyloxy)carbonyl)azepane-4-carboxylic acid (200 mg, 1.0 eq.) and Boc₂O (656 mg, 5.0 eq.) in EtOAc (2 mL) was added Pd/C (20.0 mg, 10% purity, 0.03 eq.) under N₂. The suspension was degassed under vacuum and purged with H₂ several times. The reaction was stirred under H₂ (15 psi) at 20 °C for 12 hours. The mixture was filtered and purified with reversed phase flash chromatography [C18, 0.1% formic acid condition] to afford the title compound (100 mg, 53% yield) as yellow liquid; LCMS (ESI, M+23): m/z=321.1

Step D. N-acetyl-N-methylazepane-4-carboxamide: To a solution of tert-butyl 4-(acetyl(methyl)carbamoyl)azepane-1-carboxylate (90 mg, 1.0 eq.) in DCM (0.5 mL) was added TFA (1.54 g, 45 eq.). The reaction was stirred at 20 °C for 0.5 hour. The mixture was concentrated to afford the title compound (60 mg, crude) as yellow liquid; LCMS (ESI, M+1): m/z=199.3.

Step E. tert-butyl (4-(4-(4-(acetyl(methyl)carbamoyl)azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 eq.), DIEA (50.0 mg, 2.5 eq.) and DMAP (378 μg, 0.02 eq.) in DCM (1 mL) was added 2-nitrobenzenesulfonyl chloride (37.7 mg, 1.1 eq.) at 0 °C. The reaction was stirred at 0 °C for 0.5 hour. N-acetyl-N-methylazepane-4-carboxamide (58.0 mg, 1.2 equiv, TFA) was added to above mixture. The reaction was stirred at 20 °C for 12 hours. The mixture was diluted with water (4 mL) and extracted with DCM (3×3 mL). The combined organic layers were dried over anhydrous sodium sulfate, concentrated, and purified with reversed phase flash chromatography [C18, 0.1% NH₃·H₂O condition] to afford the title compound (80 mg, 61% yield) as yellow solid; LCMS (ESI, M+1): m/z=826.3.

Step F. N-acetyl-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylazepane-4-carboxamide: To a mixture of tert-butyl (4-(4-(4-(acetyl(methyl)carbamoyl)azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (75.0 mg, 1.0 eq.) in DCM (0.25 mL) was added TFA (767 mg, 74 eq.). The reaction was stirred at 20 °C for 0.5 hour. The mixture was basified with saturated NaHCO₃ aqueous solution (10 mL) and extracted with ethyl acetate (3×3 mL). The combined organic layers were dried over anhydrous sodium sulfate, concentrated and purified with reversed phase flash chromatography [C18, 0.1% formic acid condition] to afford the title compound (24.1 mg, 36% yield) as off-white solid; ¹H NMR (400 MHz, METHANOL-d₄)_{8=8.10}-8.00 (m, 1H), 7.21 (dd, J=5.2, 8.4 Hz, 1H), 7.04 (t, J=8.8 Hz, 1H), 5.51-5.26 (m, 1H), 4.48-4.32 (m, 2H), 4.31-4.21 (m, 2H), 4.00-3.83 (m, 2H), 3.56-3.36 (m, 4H), 3.21 (d, J=2.4 Hz, 3H), 3.18-3.11 (m, 1H), 2.51-2.37 (m, 1H), 2.35 (d, J=2.0 Hz, 3H), 2.33-2.25 (m, 2H), 2.24-2.14 (m, 3H), 2.13-2.03 (m, 4H), 2.00-1.90 (m, 1H), 1.75-1.59 (m, 1H); LCMS (ESI, M+1): m/z=726.3.

### Example 256

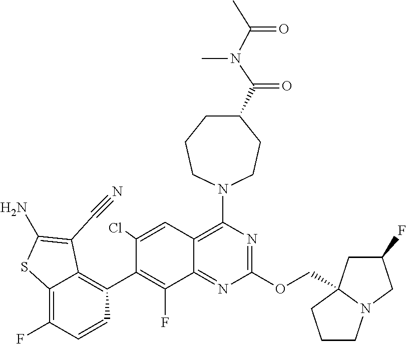

### (S)—N-acetyl-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylazepane-4-carboxamide

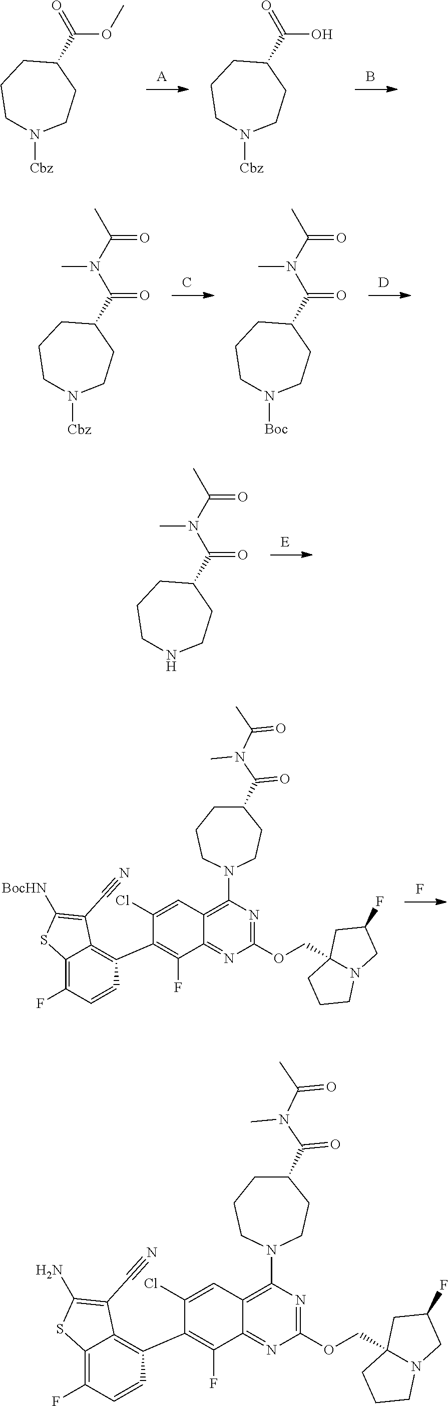

Step A. (S)-1-((benzyloxy)carbonyl)azepane-4-carboxylic acid: To a mixture of 1-benzyl 4-methyl (S)-azepane-1,4-dicarboxylate (3.00 g, 1.0 eq.) in THF (12 mL), H₂O (12 mL) and MeOH (8 mL) was added LiOH·H₂O (1.30 g, 3.0 eq.). The reaction was stirred at 20 °C for 2 hours. The mixture was filtered and purified with reversed phase flash chromatography [C18, 0.1% formic acid condition] to afford the title compound (2.85 g, 99% yield) as white solid; LCMS (ESI, M+1): m/z=278.0.

Step B. benzyl(S)-4-(acetyl(methyl)carbamoyl)azepane-1-carboxylate: To a mixture of (S)-1-((benzyloxy)carbonyl)azepane-4-carboxylic acid (1.35 g, 1.0 eq.), N-methylacetamide (712 mg, 2.0 eq.) and CMPI (3.73 g, 3.0 eq.) in 2-methyltetrahydrofuran (26 mL) was added DIEA (3.15 g, 5.0 eq.). The reaction was stirred at 80 °C for 3 hours. The mixture was concentrated and purified with reversed phase flash chromatography [C18, 0.1% formic acid condition] to afford the title compound (2.50 g, 76% yield) as yellow liquid; LCMS (ESI, M+1): m/z=333.2.

Step C. tert-butyl (S)-4-(acetyl(methyl)carbamoyl)azepane-1-carboxylate: To a mixture of benzyl(S)-4-(acetyl(methyl)carbamoyl)azepane-1-carboxylate (500 mg, 1.0 eq.) and Boc₂O (3.28 g, 10 eq.) in EtOAc (5 mL) was added Pd/C (50 mg, 10% purity, 0.03 eq.) under N₂. The suspension was degassed under vacuum and purged with H₂ several times. The reaction was stirred under H₂ (15 psi) at 20 °C for 12 hours. The mixture was filtered and the filter was concentrated to afford the title compound (450 mg, 98% yield) as yellow liquid; LCMS (ESI, M−55): m/z=243.2.

Step D. (S)—N-acetyl-N-methylazepane-4-carboxamide: To a solution of tert-butyl (S)-4-(acetyl(methyl)carbamoyl)azepane-1-carboxylate (400 mg, 1.0 eq.) in DCM (1 mL) was added TFA (2.73 g, 18 eq.). The reaction was stirred at 20 °C for 0.5 hour. The mixture was concentrated to afford the title compound (420 mg, crude, TFA) as a yellow liquid; LCMS (ESI, M+1): m/z=199.2.

Step E. tert-butyl (4-((S)-4-((S)-4-(acetyl(methyl)carbamoyl)azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of tert-butyl (4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (250 mg, 1.0 eq.), DIEA (250 mg, 5.0 eq.) and DMAP (1.89 mg, 0.04 eq.) in DCM (2.5 mL) was added 2-nitrobenzenesulfonyl chloride (111 mg, 1.3 eq.). The reaction was stirred at 0 °C for 1 hour. And then (S)—N-acetyl-N-methylazepane-4-carboxamide (402 mg, 3.4 equiv, TFA) was added to above mixture. The reaction was stirred at 20 °C for 12 hours. The mixture was diluted with water (1 mL) and extracted with DCM (3×1 mL). The combined organic layers were dried over anhydrous sodium sulfate, concentrated, and purified with reversed phase flash chromatography [C18, 0.1% NH₃·H₂O condition] to afford the title compound (250 mg, 77% yield) as yellow solid; LCMS (ESI, M+1): m/z=826.4.

Step F. (S)—N-acetyl-1-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methylazepane-4-carboxamide: To a solution of tert-butyl (4-((S)-4-((S)-4-(acetyl(methyl)carbamoyl)azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (230 mg, 1.0 eq.) in DCM (1 mL) was added TFA (3.07 g, 97 eq.). The reaction was stirred at 20 °C for 0.5 hour. The mixture was concentrated. The residue was diluted with saturated NaHCO₃ aqueous solution (8 mL) and extracted with DCM (3 mL×3). The combined organic phase was dried over anhydrous sodium sulfate, filtered, concentrated and purified with reversed phase flash chromatography [C18, 0.1% formic acid condition] to afford the title compound (105 mg, 50% yield) as off-white solid; ¹H NMR (400 MHz, METHANOL-d₄) δ=8.54 (s, 1H), 8.05 (d, J=1.2 Hz, 1H), 7.21 (dd, J=5.2, 8.4 Hz, 1H), 7.09-7.00 (m, 1H), 5.51-5.29 (m, 1H), 4.46 (d, J=11.2 Hz, 1H), 4.41-4.21 (m, 3H), 4.01-3.82 (m, 2H), 3.63-3.50 (m, 1H), 3.50-3.43 (m, 2H), 3.42-3.36 (m, 1H), 3.22 (s, 3H), 3.19-3.11 (m, 1H), 2.51-2.39 (m, 1H), 2.35 (s, 4H), 2.29-2.13 (m, 4H), 2.13-1.89 (m, 5H), 1.77-1.61 (m, 1H); SFC [Column: Chiralpak AS-3 50×4.6 mm I.D., 3 μm; Mobile phase: Phase A for CO₂ and Phase B for IPA (0.05% DEA); Gradient elution: 30% IPA (0.05% DEA) in CO₂; Flow rate: 3 mL/minute; Detector: PDA; Column Temp: 35 °C; Back Pressure: 100 Bar]; LCMS (ESI, M+1): m/z=726.3; RT=1.791 minutes.

### Example 257

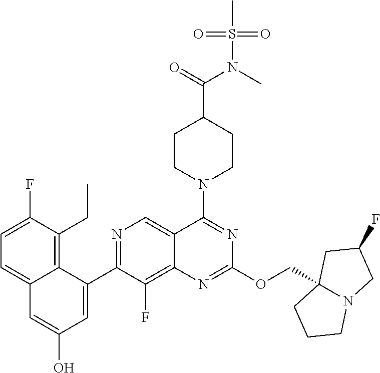 [SMILES: CCc1c(F)ccc2cc(O)cc(-c3ncc4c(N5CCC(C(=O)N(C)S(C)(=O)=O)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(methylsulfonyl)piperidine-4-carboxamide

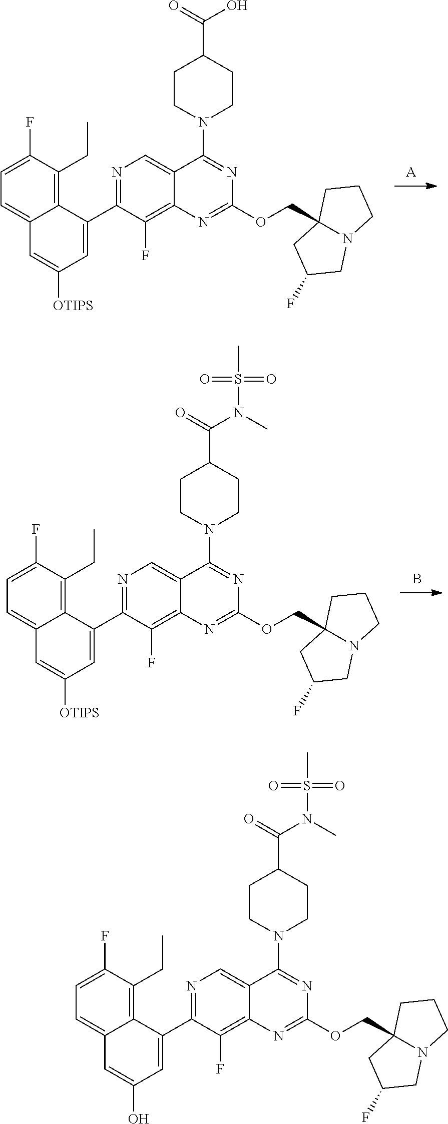

Step A. 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(methylsulfonyl)piperidine-4-carboxamide: To a solution of 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (100 mg, 1.0 eq.), N-methylmethanesulfonamide (70.1 mg, 5.0 eq.) in 2-methyltetrahydrofuran (3.0 mL) was added 2-chloro-1-methyl-pyridin-1-ium;iodide (164 mg, 5.0 eq.). The reaction was stirred at 80 °C for 12 hours. The mixture was diluted with H₂O (10 mL) and extracted with EtOAc (3×5 mL). The combined organic layers were washed with brine (10 mL), dried over anhydrous sodium sulfate, concentrated and purified by reverse phase flash chromatography [C18, 0.1% formic acid condition] to afford the title compound (60 mg, 53% yield) as yellow solid; ¹H NMR (400 MHz, CHLOROFORM-d₃) δ=9.03-8.99 (m, 1H), 7.61 (dd, J=5.6, 8.8 Hz, 1H), 7.32 (d, J=2.4 Hz, 1H), 7.25-7.20 (m, 1H), 7.11 (d, J=2.4 Hz, 1H), 5.43-5.22 (m, 1H), 4.71-4.59 (m, 2H), 4.43-4.21 (m, 2H), 3.63-3.44 (m, 2H), 3.44-3.36 (m, 2H), 3.35 (s, 3H), 3.27 (s, 3H), 3.25 (br s, 1H), 3.08-2.97 (m, 1H), 2.56-2.43 (m, 1H), 2.40-2.25 (m, 2H), 2.24-2.15 (m, 2H), 2.10 (br d, J=3.2 Hz, 4H), 2.01 (s, 1H), 2.00-1.76 (m, 3H), 1.36-1.29 (m, 3H), 1.12 (d, J=7.2 Hz, 18H), 0.84 (t, J=7.2 Hz, 3H); LCMS (ESI, M+1): m/z=869.7.

Step B. 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(methylsulfonyl)piperidine-4-carboxamide: A mixture of 1-(7-(8-ethyl-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-N-(methylsulfonyl)piperidine-4-carboxamide (50.0 mg, 1.0 eq.) in pyridine; hydrofluoride (1 mL) was stirred at 25 °C for 10 minutes under nitrogen atmosphere. The mixture was diluted with H₂O (2 mL) and extracted with EtOAc (3×1 mL). The combined organic layers were washed with brine (2 mL), dried over anhydrous sodium sulfate, concentrated and purified with prep-HPLC [Phenomenex luna C18 150×25 mm×10 μm; A: water (FA); B: ACN, B %: 18%-48% over 10 min] to afford the title compound (5.19 mg, 97% yield) as white solid; ¹H NMR (400 MHz, CHLOROFORM-d₃) δ=8.94-8.89 (m, 1H), 7.60-7.50 (m, 1H), 7.21 (br s, 1H), 7.19-7.14 (m, 1H), 7.03-6.89 (m, 1H), 5.51-5.25 (m, 1H), 4.66-4.42 (m, 4H), 3.79-3.55 (m, 1H), 3.34 (br s, 2H), 3.31 (s, 3H), 3.26 (s, 3H), 3.24 (br s, 1H), 3.15-3.06 (m, 1H), 2.63-2.40 (m, 2H), 2.39-2.18 (m, 3H), 2.17-1.89 (m, 9H), 0.85-0.76 (m, 3H); LCMS (ESI, M+1): m/z=713.4.

### Example 258

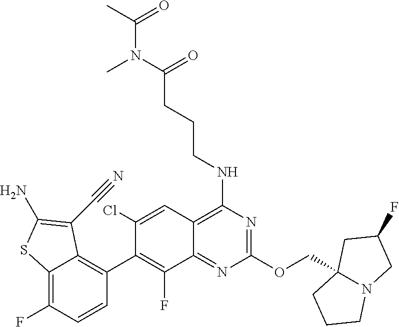 [SMILES: CC(=O)N(C)C(=O)CCCNc1nc(OC[C@@]23CCCN2C[C@H](F)C3)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)-N-methylbutanamide

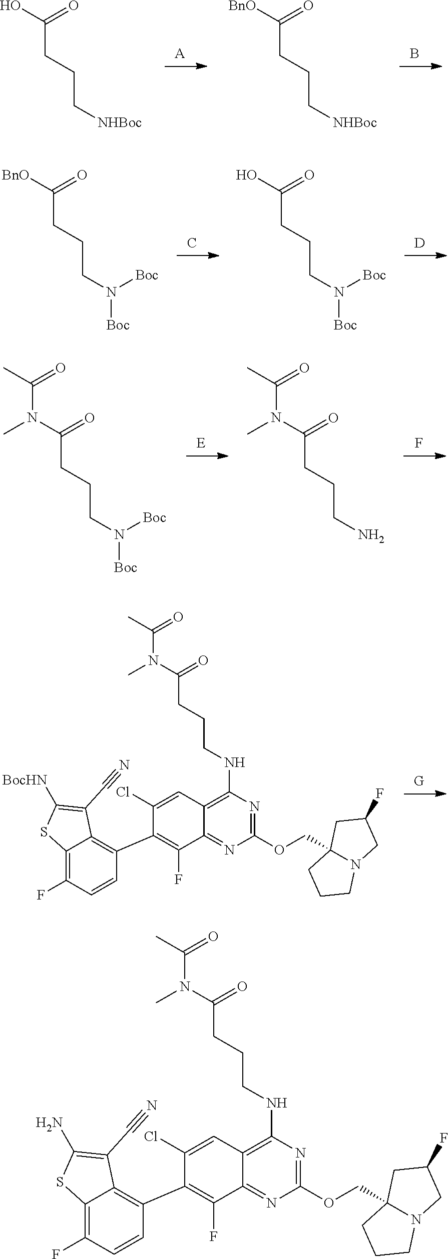

Step A. benzyl 4-((tert-butoxycarbonyl)amino)butanoate: To a solution of 4-((tert-butoxycarbonyl)amino)butanoic acid (3.50 g, 1.0 eq.) and BnOH (1.86 g, 1.0 eq.) in DCM (20 mL) were added DCC (5.33 g, 1.5 eq.) and DMAP (210 mg, 0.1 eq.). The reaction was stirred at 25 °C for 2 hours. The mixture was diluted with water (50 mL) and extracted with DCM (3× 30 mL). The combined organic layers were washed with brine (50 mL), dried over anhydrous sodium sulfate, concentrated and purified with column chromatography [SiO₂, petroleum ether/ethyl acetate=50/1 to 10/1] to afford the title compound (3.40 g, 67% yield) as colorless oil; ¹H NMR (400 MHz, METHANOL-d₄) δ=7.36-7.29 (m, 5H), 5.11 (s, 2H), 3.07 (t, J=6.8 Hz, 2H), 2.39 (t, J=7.6 Hz, 2H), 1.77 (quin, J=7.2 Hz, 2H), 1.43 (s, 9H).

Step B. benzyl 4-[bis(tert-butoxycarbonyl)amino]butanoate: To a solution of benzyl 4-((tert-butoxycarbonyl)amino)butanoate (3.40 g, 1.0 eq.) in MeCN (20 mL) were added Boc₂O (5.06 g, 2.0 eq.), DMAP (142 mg, 0.1 eq.) and TEA (3.52 g, 3.0 eq.). The reaction was stirred at 80 °C for 1 hour. The mixture was diluted with water (50 mL) and extracted with ethyl acetate (3×30 mL). The combined organic layers were washed with brine (50 mL), dried over anhydrous sodium sulfate, concentrated, and purified with column chromatography [SiO₂, petroleum ether/ethyl acetate=50/1 to 10/1] to afford the title compound (4.00 g, 85% yield) as yellow oil; LCMS (ESI, M+23): m/z=416.2.

Step C. 4-[bis(tert-butoxycarbonyl)amino]butanoic acid: A mixture of benzyl 4-[bis(tert-butoxycarbonyl)amino]butanoate (4.00 g, 1.0 eq.) and Pd/C (500 mg, 10% purity) in MeOH (20 mL) was degassed and purged with H₂ for 3 times. The reaction was stirred at 25 °C for 1 hour under H₂ (15 psi) pressure. The mixture was filtered, and the filtrate was concentrated to afford the title compound (2.95 g, 96% yield) as yellow oil; ¹H NMR (400 MHz, METHANOL-d₄) δ=3.64 (t, J=6.8 Hz, 2H), 2.31 (t, J=7.2 Hz, 2H), 1.86 (quin, J=7.2 Hz, 2H), 1.51 (s, 18H).

Step D. tert-butyl N-[4-[acetyl(methyl)amino]-4-oxo-butyl]-N-tert-butoxycarbonyl-carbamate: To a solution of 4-[bis(tert-butoxycarbonyl)amino]butanoic acid (2.90 g, 1.0 eq.) and N-methylacetamide (3.49 g, 5.0 eq.) in 2-MeTHF (40 mL) were added DIEA (6.18 g, 5.0 eq.) and CMPI (12.2 g, 5.0 eq.). The reaction was stirred at 80 °C for 1 hour. The mixture was diluted with water (60 mL) and extracted with ethyl acetate (3×30 mL). The combined organic layers were washed with brine (50 mL), dried over anhydrous sodium sulfate, concentrated and purified with prep-HPLC [column: Phenomenex luna C18 150×40 mm×15 μm; mobile phase: water (FA)-ACN; gradient: 45%-75% B over 15 min] to afford the title compound (680 mg, 20% yield) as brown oil; ¹H NMR (400 MHz, METHANOL-d₄) δ=3.66 (t, J=6.8 Hz, 2H), 3.20 (s, 3H), 2.75 (t, J=6.8 Hz, 2H), 2.39 (s, 3H), 1.90 (t, J=6.8 Hz, 2H), 1.51 (s, 18H).

Step E. N-acetyl-4-amino-N-methylbutanamide: To a solution of tert-butyl N-[4-[acetyl(methyl)amino]-4-oxo-butyl]-N-tert-butoxycarbonyl-carbamate (680 mg, 1.0 eq.) in DCM (4 mL) was added TFA (2 mL). The reaction was stirred at 25 °C for 1 hour. The mixture was concentrated, dissolved in MeOH (2 mL), neutralized with solid NaHCO₃ and filtered. The filtrate was concentrated to afford the title compound (600 mg, crude, TFA) as brown oil; ¹H NMR (400 MHz, DMSO-d₆) δ=3.12 (s, 3H), 2.88-2.76 (m, 4H), 2.34 (s, 3H), 1.86-1.74 (m, 2H).

Step F. tert-butyl (4-(6-chloro-8-fluoro-2-(((2R,7aS)-2-fluorotetrahydro-1H-pyrrolizin-7a(5H)-yl)methoxy)-4-((4-(N-methylacetamido)-4-oxobutyl)amino)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (300 mg, 1.0 eq.) and N-acetyl-4-amino-N-methylbutanamide (253 mg, 2.0 equiv, TFA) in DMF (3 mL) were added PyBOP (362 mg, 1.5 eq.) and DIEA (300 mg, 5.0 eq.). The reaction was stirred at 40 °C for 1 hour. The mixture was diluted with water (15 mL) and extracted with ethyl acetate (3×15 mL). The combined organic layers were washed with brine (20 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 50%-80% B over 15 min] to afford the title compound (230 mg, 59% yield) as brown solid; LCMS (ESI, M+1): m/z=786.3.

Step G. N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)-N-methylbutanamide: To a solution of tert-butyl (4-(6-chloro-8-fluoro-2-(((2R,7aS)-2-fluorotetrahydro-1H-pyrrolizin-7a(5H)-yl)methoxy)-4-((4-(N-methylacetamido)-4-oxobutyl)amino)quinazolin-7-yl)-3-cyano-7-fluorobenzo[b]thiophen-2-yl)carbamate (150 mg, 1.0 eq.) in DCM (3 mL) was added TFA (1 mL). The reaction was stirred at 25 °C for 1 hour. The mixture was concentrated, redissolved in MeCN (2 mL) and neutralized with DIEA at −40 °C. Then the mixture was diluted with water (10 mL) and extracted with ethyl acetate (3×10 mL). The combined organic layers were washed with brine (10 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 38%-68% B over 15 min] to afford the title compound (57.3 mg, 42% yield) as off-white solid; ¹H NMR (400 MHz, DMSO-d₆) δ=8.54 (br t, J=5.2 Hz, 1H), 8.30 (s, 1H), 8.09 (s, 2H), 7.26-7.19 (m, 1H), 7.18-7.10 (m, 1H), 5.37-5.16 (m, 1H), 4.13-4.04 (m, 1H), 3.98 (dd, J=2.8, 10.4 Hz, 1H), 3.64-3.49 (m, 2H), 3.13 (s, 3H), 3.11-3.05 (m, 2H), 3.04-3.00 (m, 1H), 2.87-2.78 (m, 3H), 2.33 (s, 3H), 2.19-2.10 (m, 1H), 2.06 (br d, J=12.4 Hz, 1H), 2.02-1.92 (m, 3H), 1.86-1.72 (m, 3H); LCMS (ESI, M+1): m/z=686.4.

### Example 259

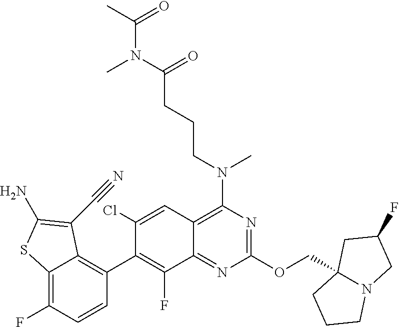 [SMILES: CC(=O)N(C)C(=O)CCCN(C)c1nc(OC[C@@]23CCCN2C[C@H](F)C3)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methylbutanamide

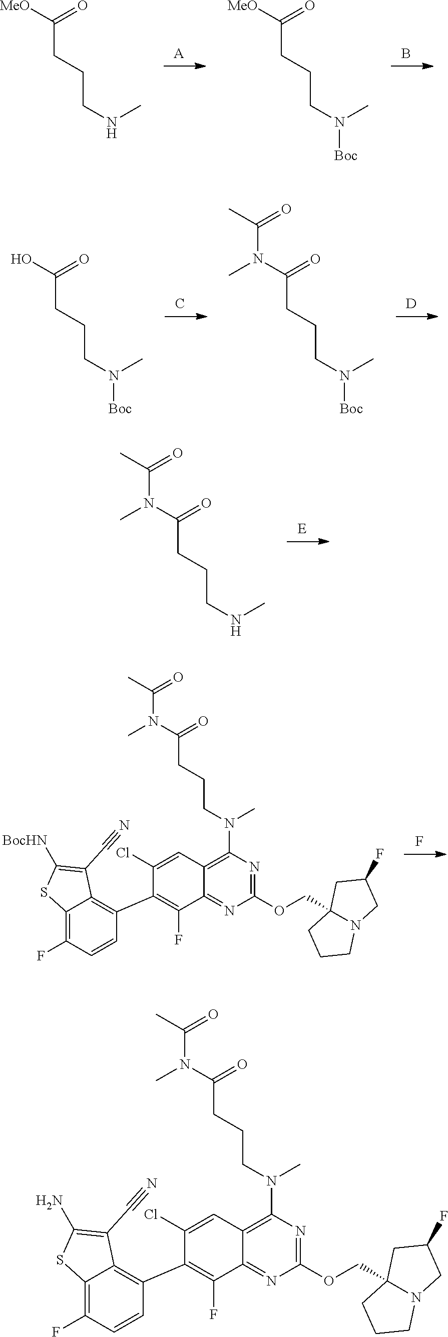

Step A. methyl 4-((tert-butoxycarbonyl)(methyl)amino)butanoate; To a solution of methyl 4-(methylamino)butanoate (3.00 g, 1.0 equiv, HCl) and DIEA (15.6 mL, 5.0 eq.) in ethanol (60 mL) was added Boc₂O (5.86 g, 1.5 eq.). The reaction was stirred at 25 °C for 1 hour. The mixture was diluted with water (50 mL) and extracted with ethyl acetate (2×50 mL). The combined organic layers were washed with brine (50 mL), dried over anhydrous sodium sulfate, and concentrated to afford the title compound (6.00 g, crude) as colorless oil; LCMS (ESI, M+23): m/z=254.2.

Step B. 4-((tert-butoxycarbonyl)(methyl)amino)butanoic acid: To a solution of methyl 4-((tert-butoxycarbonyl)(methyl)amino)butanoate (6.00 g, 1.0 eq.) in MeOH (30 mL) was added LiOH·H₂O (30 mL, 2M). The reaction was stirred at 25 °C for 1 hour. The mixture was concentrated, diluted with water (100 mL) and extracted with ethyl acetate (2×150 mL). The organic solvent was acidified with solid citric acid and filtered. The filtrate was washed with brine (120 mL), dried over anhydrous sodium sulfate, concentrated, and purified with column chromatography [SiO₂, petroleum ether/ethyl acetate=20/1 to 2/1] to afford the title compound (2.20 g, 39% yield) as white solid; LCMS (ESI, M+23): m/z=240.2.

Step C. tert-butyl methyl (4-(N-methylacetamido)-4-oxobutyl)carbamate; To a solution of 4-((tert-butoxycarbonyl)(methyl)amino)butanoic acid (2.00 g, 1.0 eq.) and N-methylacetamide (3.36 g, 5.0 eq.) in 2-MeTHF (40 mL) were added CMPI (11.8 g, 5.0 eq.) and DIEA (8.0 mL, 5.0 eq.). The reaction was stirred at 80 °C for 3 hours. The mixture was diluted with water (80 mL) and extracted with ethyl acetate (2×60 mL). The combined organic layers were washed with brine (50 mL), dried over anhydrous sodium sulfate, concentrated and purified with prep-HPLC [column: Phenomenex luna C18 (250×70 mm, 10 μm); mobile phase: water (FA)-ACN; gradient: 20%-50% B over 25 min] to afford the title compound (384 mg, 15% yield) as yellow oil; LCMS (ESI, M+23): m/z=295.2.

Step D. N-acetyl-N-methyl-4-(methylamino)butanamide: To a solution of tert-butyl methyl (4-(N-methylacetamido)-4-oxobutyl)carbamate (384 mg, 1.0 eq.) in DCM (3 mL) was added TFA (1 mL). The reaction was stirred at 20 °C for 1 hour. The mixture was concentrated, dissolved in DCM (3 mL), neutralized with DIEA at −40 °C, and concentrated to afford the title compound (250 mg, crude) as orange gum; LCMS (ESI, M+1): m/z=173.0.

Step E. tert-butyl (4-(6-chloro-8-fluoro-2-(((2R,7aS)-2-fluorotetrahydro-1H-pyrrolizin-7a(5H)-yl)methoxy)-4-(methyl (4-(N-methylacetamido)-4-oxobutyl)amino)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (110 mg, 1.0 eq.) and N-acetyl-N-methyl-4-(methylamino)butanamide (244 mg, 5.0 equiv, TFA) in DMF (2 mL) were added PyBOP (443 mg, 5.0 eq.) and DIEA (0.3 mL, 10 eq.). The reaction was stirred at 40 °C for 1 hour. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge BEH C18 250×50 mm×10 μm; mobile phase: water (NH₄HCO₃)-ACN; gradient: 50%-80% B over 30 min] to afford the title compound (50.0 mg, 36% yield) as yellow solid; LCMS (ESI, M+1): m/z=800.4.

Step F. N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methylbutanamide: To a solution of tert-butyl (4-(6-chloro-8-fluoro-2-(((2R,7aS)-2-fluorotetrahydro-1H-pyrrolizin-7a(5H)-yl)methoxy)-4-(methyl (4-(N-methylacetamido)-4-oxobutyl)amino)quinazolin-7-yl)-3-cyano-7-fluorobenzo[b]thiophen-2-yl)carbamate (50.0 mg, 1.0 eq.) in DCM (1.5 mL) was added TFA (0.5 mL). The reaction was stirred at 25 °C for 2 hours. The mixture was concentrated, dissolved in DMF (2 mL), neutralized with DIEA at −40 °C, and diluted with water (10 mL). The mixture was extracted with ethyl acetate (2×15 mL). The combined organic layers were washed with brine (10 mL), dried over anhydrous sodium sulfate, concentrated and purified with prep-HPLC [column: Waters Xbridge 150×25 mm×5 μm; mobile phase: water (NH₄HCO₃)-ACN; gradient: 48%-78% B over 9 min] to afford the title compound (10.2 mg, 21% yield) as white solid; 1HNMR (400 MHz, METHANOL-d₄) δ=8.12 (s, 1H), 7.20 (dd, J=5.2, 8.4 Hz, 1H), 7.08-7.00 (m, 1H), 5.43-5.18 (m, 1H), 4.33-4.18 (m, 2H), 3.99-3.83 (m, 2H), 3.52 (s, 3H), 3.29-3.12 (m, 6H), 3.04-2.96 (m, 1H), 2.90 (t, J=6.8 Hz, 2H), 2.37 (s, 3H), 2.34-2.12 (m, 5H), 2.02-1.85 (m, 3H); LCMS (ESI, M+1): m/z=700.2.

### Example 260

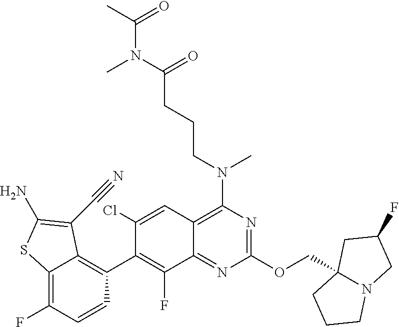

### N-acetyl-4-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methylbutanamide

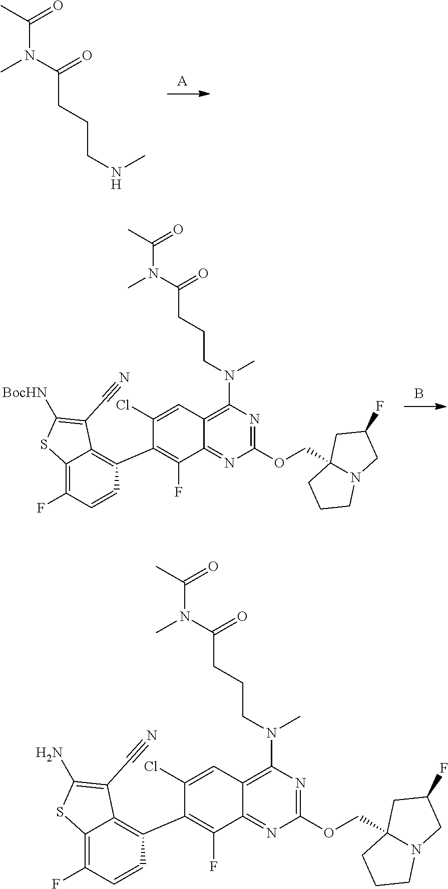

Step A. tert-butyl (4-((S)-6-chloro-8-fluoro-2-(((2R,7aS)-2-fluorotetrahydro-1H-pyrrolizin-7a(5H)-yl)methoxy)-4-(methyl (4-(N-methylacetamido)-4-oxobutyl)amino)quinazolin-7-yl)-3-cyano-7-fluorobenzo[b]thiophen-2-yl)carbamate: To a solution of tert-butyl (4-((S)-6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (2.60 g, 1.0 eq.) and N-acetyl-N-methyl-4-(methylamino)butanamide (5.76 g, 5.0 eq.) in DMF (30 mL) were added PyBOP (10.5 g, 5.0 eq.) and DIEA (7.01 mL, 10 eq.). The reaction was stirred at 40 °C for 1 hour. The mixture was diluted with water (40 mL) and extracted with ethyl acetate (2×60 mL). The combined organic layers were washed with brine (40 mL), dried over anhydrous sodium sulfate, concentrated and purified with prep-HPLC [column: Kromasil Eternity XT 250×80 mm×10 um; mobile phase: water (NH₄HCO₃)-ACN; gradient: 60%-90% B over 30 min] to afford the title compound (2.46 g, 76% yield) as yellow solid; LCMS (ESI, M+1): m/z=800.2.

Step B. N-acetyl-4-(((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methylbutanamide: To a solution of tert-butyl (4-((S)-6-chloro-8-fluoro-2-(((2R,7aS)-2-fluorotetrahydro-1H-pyrrolizin-7a(5H)-yl)methoxy)-4-(methyl (4-(N-methylacetamido)-4-oxobutyl)amino)quinazolin-7-yl)-3-cyano-7-fluorobenzo[b]thiophen-2-yl)carbamate (1.50 g, 1.0 eq.) in DCM (30 mL) was added TFA (10 mL). The reaction was stirred at 25 °C for 2 hours. The mixture was concentrated, dissolved in DMF (10 mL), neutralized with DIEA at −40 °C, then diluted with water (40 mL) and extracted with ethyl acetate (2×60 mL). The combined organic layers were washed with brine (30 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 42%-72% B over 15 min] to afford the title compound (736 mg, 55% yield) as off-white solid; 1HNMR (400 MHz, METHANOL-d₄) δ=8.12 (s, 1H), 7.20 (dd, J=5.2, 8.4 Hz, 1H), 7.08-7.00 (m, 1H), 5.43-5.18 (m, 1H), 4.33-4.18 (m, 2H), 3.99-3.83 (m, 2H), 3.52 (s, 3H), 3.29-3.12 (m, 6H), 3.04-2.96 (m, 1H), 2.90 (t, J=6.8 Hz, 2H), 2.37 (s, 3H), 2.34-2.12 (m, 5H), 2.02-1.85 (m, 3H); LCMS (ESI, M+1): m/z=700.2.

### Example 261

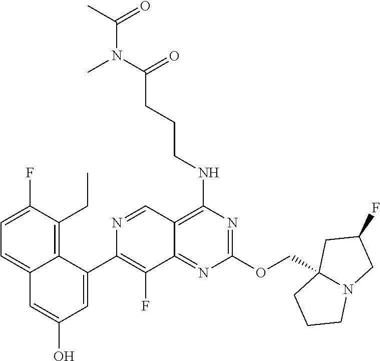 [SMILES: CCc1c(F)ccc2cc(O)cc(-c3ncc4c(NCCCC(=O)N(C)C(C)=O)nc(OC[C@@]56CCCN5C[C@H](F)C6)nc4c3F)c12]

### N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-N-methylbutanamide

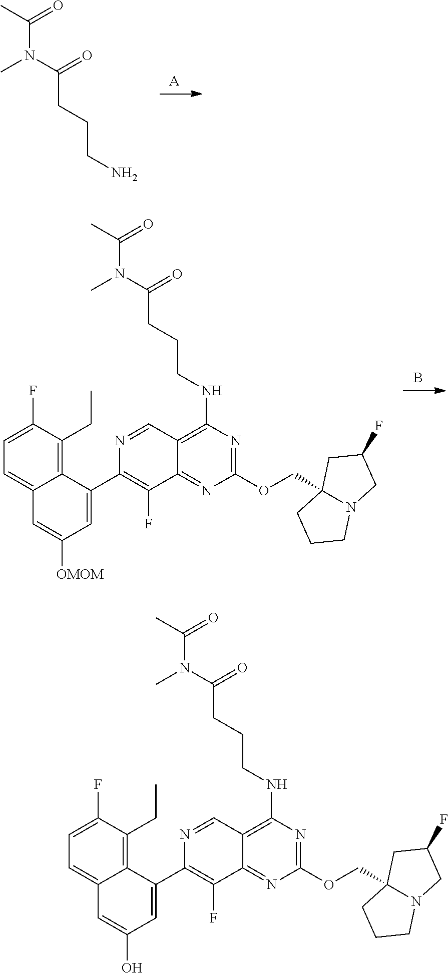

Step A. N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-N-methylbutanamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (64.0 mg, 1.0 eq.) in DMF (2 mL) was added PyBOP (72.1 mg, 1.2 eq.) and DIEA (44.7 mg, 3.0 eq.). The mixture was stirred at 25 °C for 10 minutes, and then N-acetyl-4-amino-N-methylbutanamide (21.9 mg, 1.2 eq.) was added dropwise. The reaction was stirred at 25 °C for 0.5 hour. The mixture was filtered, concentrated, and purified with prep-HPLC [Waters Xbridge 150×25 mm×5 μm; A: water (ammonia hydroxide v/v); B: ACN, B %: 38%-68% over 10 min] to afford the title compound (20.0 mg, 24% yield over two steps) as white solid; LCMS (ESI, M+1): m/z=695.4.

Step B. N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-N-methylbutanamide: To a solution of N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)-N-methylbutanamide (15.0 mg, 1.0 eq.) in DCM (1 mL) was added TFA (767 mg, 311.8 eq.) at 0 °C. The reaction was stirred at 25 °C for 1 hour. The mixture was concentrated and diluted with MeOH (2 mL) and neutralized with solid NaHCO₃. Then the mixture was filtered, concentrated and purified with prep-HPLC [Phenomenex luna C18 150×25 mm×10 μm; A: water (FA); B: ACN, B %: 20%-50% over 9 min] to afford the title compound (8.39 mg, 53% yield over two steps, HCOOH); LCMS (ESI, M+1): m/z=651.4.

### Example 262

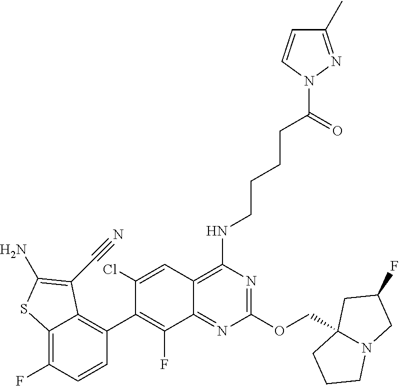 [SMILES: Cc1ccn(C(=O)CCCCNc2nc(OC[C@@]34CCCN3C[C@H](F)C4)nc3c(F)c(-c4ccc(F)c5sc(N)c(C#N)c45)c(Cl)cc23)n1]

### 2-amino-4-(6-chloro-8-fluoro-2-(((2R,7aS)-2-fluorotetrahydro-1H-pyrrolizin-7a(5H)-yl)methoxy)-4-((5-(3-methyl-1H-pyrazol-1-yl)-5-oxopentyl)amino)quinazolin-7-yl)-7-fluorobenzo[b]thiophene-3-carbonitrile

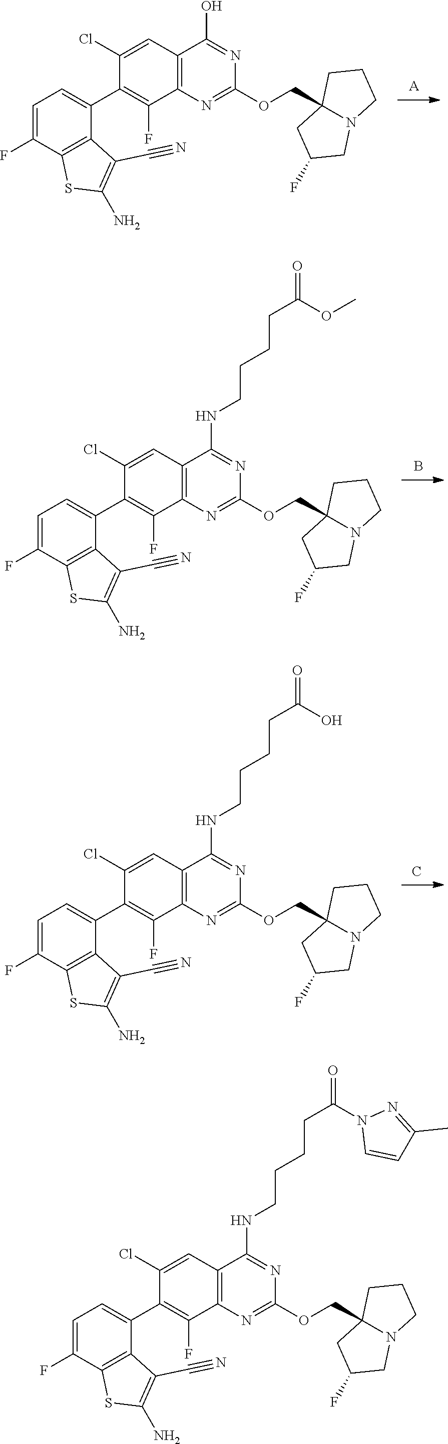

Step A. methyl 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)pentanoate: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (200 mg, 1.0 eq.) in N,N-dimethylformamide (2 mL) were added PYBOP (229 mg, 1.2 eq.) and diisopropylethylamine (237 mg, 5.0 eq.) at 25 °C. The reaction was stirred at 25 °C for 0.5 hour. Then methyl 5-aminopentanoate (92 mg, 1.2 equiv, HCl) was added to the reaction, and the reaction was stirred at 25 °C for 5.5 hours. The mixture was quenched with water (5 mL) at 25 °C and extracted with ethyl acetate (3×5 mL). The combined organic layers were washed with brine (3×5 mL), dried over anhydrous sodium sulfate, filtered and concentrated to afford the title compound (150 mg, 52% yield) as yellow solid; LCMS (ESI, M+1): m/z=659.3.

Step B. 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)pentanoic acid: To a solution of methyl 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)pentanoate (100 mg, 1.0 eq.) in methanol (1 mL) and tetrahydrofuran (1 mL) was added lithium hydroxide (2 M, 0.2 mL) at 25 °C. The reaction was stirred at 25 °C for 2 hours. The mixture was filtered and purified with prep-HPLC [Waters Xbridge 150×25 mm×5 μm; A: water (10 mM NH₄HCO₃), B: ACN, B %: 20% to 50% over 9 min] to afford the title compound (55.0 mg, 61% yield) as off-white solid; LCMS (ESI, M+1): m/z=645.3.

Step C 2-amino-4-(6-chloro-8-fluoro-2-(((2R,7aS)-2-fluorotetrahydro-1H-pyrrolizin-7a(5H)-yl)methoxy)-4-((5-(3-methyl-1H-pyrazol-1-yl)-5-oxopentyl)amino)quinazolin-7-yl)-7-fluorobenzo[b]thiophene-3-carbonitrile: To a solution of 5-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)pentanoic acid (30.0 mg, 1.0 eq.) and 3-methyl-1H-pyrazole (10.0 mg, 3.0 eq.) in N,N-dimethylformamide (0.5 mL) were added EDCI (16.0 mg, 2.0 eq.), HOBt (11.0 mg, 2.0 eq.) and diisopropylethylamine (16.6 mg, 3.0 eq.) at 25 °C. The reaction was stirred at 25 °C for 3 hours. The mixture was filtered and purified with prep-HPLC (Waters Xbridge 150×25 mm×5 μm; A: water (10 mM NH₄HCO₃), B: ACN, B %: 48%-78% over 9 min] to afford the title compound (5.8 mg, 15% yield) as off-white solid; ¹H NMR (400 MHz, DMSO-d₆) δ=8.58-8.53 (m, 1H), 8.29 (s, 1H), 8.27 (d, J=2.8 Hz, 1H), 8.10 (s, 2H), 7.26-7.18 (m, 1H), 7.17-7.10 (m, 1H), 6.43 (d, J=2.8 Hz, 1H), 5.36-5.16 (m, 1H), 4.12-3.93 (m, 2H), 3.70-3.48 (m, 2H), 3.19-2.97 (m, 5H), 2.86-2.77 (m, 1H), 2.25 (s, 3H), 2.14-2.08 (m, 1H), 2.04-1.95 (m, 2H), 1.83-1.68 (m, 7H); LCMS (ESI, M+1): m/z=709.3.

### Example 263

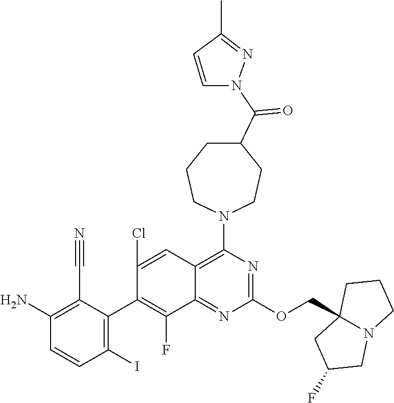 [SMILES: Cc1ccn(C(=O)C2CCCN(c3nc(OC[C@@]45CCCN4C[C@H](F)C5)nc4c(F)c(-c5c(I)ccc(N)c5C#N)c(Cl)cc34)CC2)n1]

### 6-amino-2-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl)azepan-1-yl)quinazolin-7-yl)-3-iodobenzonitrile

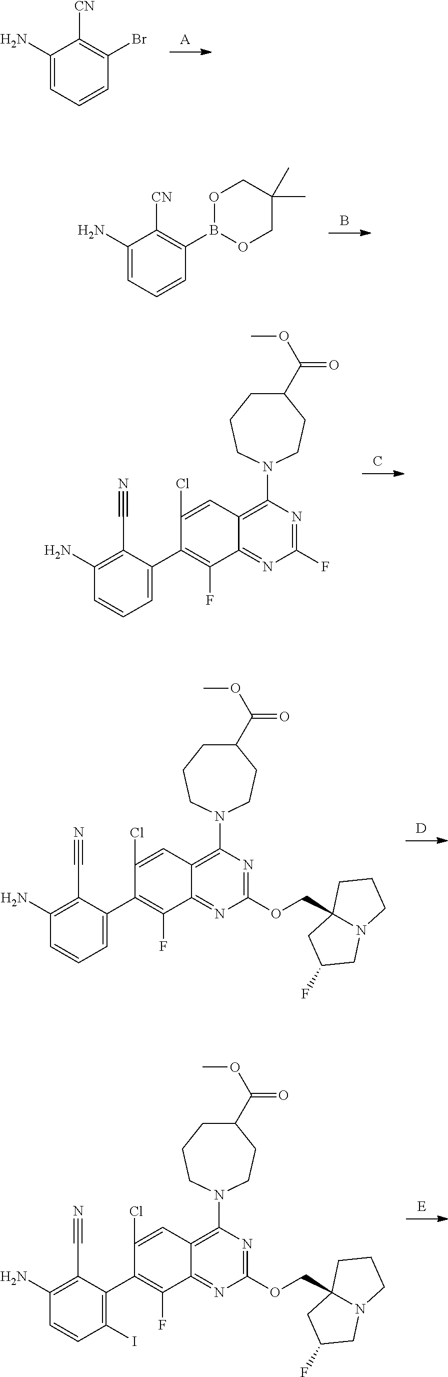

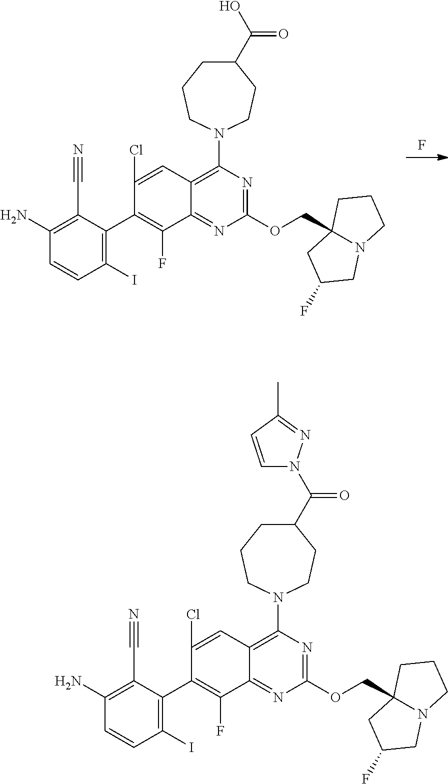

Step A. 2-amino-6-(5,5-dimethyl-1,3,2-dioxaborinan-2-yl)benzonitrile: To a mixture of 2-amino-6-bromobenzonitrile (2.00 g, 1.0 eq.), 5,5,5′,5′-tetramethyl-2,2′-bi (1,3,2-dioxaborinane) (3.44 g, 1.5 eq.) and KOAc (2.99 g, 3.0 eq.) in dioxane (20 mL) was added Pd (DPEphos) C12 (1.45 g, 0.2 eq.). The reaction was degassed and purged with nitrogen 3 times. The reaction was stirred at 100 °C for 2 hours. The mixture was filtered, concentrated, and purified by column chromatography (SiO₂, Petroleum ether/Ethyl acetate=1/0 to 5/1) to afford the title compound (7.20 g, 60% yield) as yellow solid; ¹H NMR (400 MHz, DMSO-d₆) δ=7.27-7.22 (m, 1H), 6.96-6.85 (m, 2H), 5.81 (s, 2H), 3.75 (s, 4H), 0.96 (s, 6H); LCMS (ESI, M+1): m/z=231.2.

Step B. methyl 1-(7-(3-amino-2-cyanophenyl)-6-chloro-2,8-difluoroquinazolin-4-yl)azepane-4-carboxylate: To a mixture of methyl 1-(7-bromo-6-chloro-2,8-difluoroquinazolin-4-yl)azepane-4-carboxylate (200 mg, 1.0 eq.), 2-amino-6-(5,5-dimethyl-1,3,2-dioxaborinan-2-yl)benzonitrile (212 mg, 2.0 eq.) and Cs₂CO₃ (450 mg, 3.0 eq.) in dioxane (2 mL) was added Pd(DPEphos)Cl₂ (65.9 mg, 0.2 eq.). The reaction was degassed and purged with nitrogen 3 times. The reaction was stirred at 90 °C for 3 hours. The mixture was filtered and purified with reversed phase flash chromatography [C18, 0.1% formic acid condition] to afford the title compound (600 mg, 64% yield) as pink solid; LCMS (ESI, M+1): m/z=472.2.

Step C. methyl 1-(7-(3-amino-2-cyanophenyl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a mixture of methyl 1-(7-(3-amino-2-cyanophenyl)-6-chloro-2,8-difluoroquinazolin-4-yl)azepane-4-carboxylate (223 mg, 1.2 eq.), ((2R,7aS)-2-fluorotetrahydro-1H-pyrrolizin-7a(5H)-yl)methanol (550 mg, 1.0 eq.) and 4 Å molecular sieve (50 mg) in THF (5 mL) was added t-BuONa (134 mg, 1.2 eq.). The reaction was stirred at 0 °C for 2 hours. The mixture was diluted with water (20 mL) and extracted with ethyl acetate (3×10 mL). The combined organic layers were dried over anhydrous sodium sulfate, concentrated, and purified with reversed phase flash chromatography [C18, 0.1% formic acid condition] to afford the title compound (250 mg, 33% yield) as yellow solid; LCMS (ESI, M+1): m/z=611.2.

Step D. methyl 1-(7-(3-amino-2-cyano-6-iodophenyl)-6-chloro-8-fluoro-2-(((2R,7aS)-2-fluorotetrahydro-1H-pyrrolizin-7a(5H)-yl)methoxy)quinazolin-4-yl)azepane-4-carboxylate: To a mixture of methyl 1-(7-(3-amino-2-cyanophenyl)-6-chloro-8-fluoro-2-(((2R,7aS)-2-fluorotetrahydro-1H-pyrrolizin-7a(5H)-yl)methoxy)quinazolin-4-yl)azepane-4-carboxylate (140 mg, 1.0 eq.) in AcOH (1.5 mL) was added NIS (56.7 mg, 1.1 eq.). The reaction was stirred at 40 °C for 1 hour. The mixture was filtered and purified with reversed phase flash chromatography [C18, 0.1% formic acid condition] to afford the title compound (40.0 mg, 22% yield) as yellow solid; LCMS (ESI, M+1): m/z=737.2.

Step E. 1-(7-(3-amino-2-cyano-6-iodophenyl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid: To a mixture of methyl 1-(7-(3-amino-2-cyano-6-iodophenyl)-6-chloro-8-fluoro-2-(((2R,7aS)-2-fluorotetrahydro-1H-pyrrolizin-7a(5H)-yl)methoxy)quinazolin-4-yl)azepane-4-carboxylate (30.0 mg, 1.0 eq.) in THF (0.3 mL), H₂O (0.3 mL) and MeOH (0.2 mL) was added LiOH·H₂O (5.12 mg, 3.0 eq.). The reaction was stirred at 20 °C for 2 hours. The mixture was filtered and purified with reversed phase flash chromatography [C18, 0.1% formic acid condition] to afford the title compound (30.0 mg, 99% yield) as yellow solid; LCMS (ESI, M+1): m/z=723.0.

Step F. 6-amino-2-(6-chloro-8-fluoro-2-(((2R,7aS)-2-fluorotetrahydro-1H-pyrrolizin-7a(5H)-yl)methoxy)-4-(4-(3-methyl-1H-pyrazole-1-carbonyl)azepan-1-yl)quinazolin-7-yl)-3-iodobenzonitrile: To a mixture of 1-(7-(3-amino-2-cyano-6-iodophenyl)-6-chloro-8-fluoro-2-(((2R,7aS)-2-fluorotetrahydro-1H-pyrrolizin-7a(5H)-yl)methoxy)quinazolin-4-yl)azepane-4-carboxylic acid (25 mg, 1.0 eq.), HOBt (7.01 mg, 1.5 eq.) and DIEA (13.4 mg, 3.0 eq.) in DMF (0.5 mL) were added 3-methyl-1H-pyrazole (5.68 mg, 2.0 eq.) and EDCI (9.94 mg, 1.5 eq.). The reaction was stirred at 20 °C for 12 hours. The mixture was filtered, diluted with DMF (1 mL) and purified with reversed phase flash chromatography [C18, water condition] to afford the title compound (8.61 mg, 29% yield) as white solid; ¹H NMR (400 MHz, CHLOROFORM-d) δ=8.15 (d, J=2.0 Hz, 1H), 7.92 (s, 1H), 7.80 (d, J=8.8 Hz, 1H), 6.63 (d, J=8.8 Hz, 1H), 6.27 (d, J=2.4 Hz, 1H), 5.39-5.15 (m, 1H), 4.67 (s, 2H), 4.32-4.11 (m, 4H), 4.01-3.80 (m, 3H), 3.34-3.08 (m, 3H), 3.03-2.87 (m, 1H), 2.47-2.38 (m, 1H), 2.34-2.28 (m, 4H), 2.26-2.16 (m, 4H), 1.97-1.84 (m, 4H); LCMS (ESI, M+1): m/z=787.3.

### Example 264

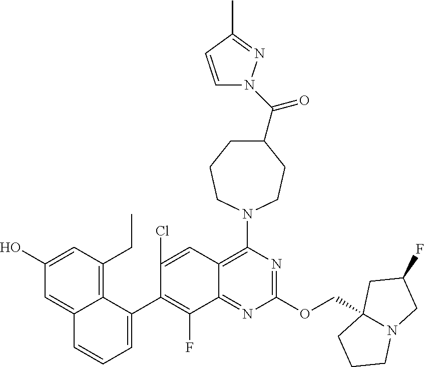 [SMILES: CCc1cc(O)cc2cccc(-c3c(Cl)cc4c(N5CCCC(C(=O)n6ccc(C)n6)CC5)nc(OC[C@@]56CCCN5C[C@H](F)C6)nc4c3F)c12]

### (1-(6-chloro-7-(8-ethyl-6-hydroxynaphthalen-1-yl)-8-fluoro-2-(((2R,7aS)-2-fluorotetrahydro-1H-pyrrolizin-7a(5H)-yl)methoxy)quinazolin-4-yl)azepan-4-yl)(3-methyl-1H-pyrazol-1-yl)methanone

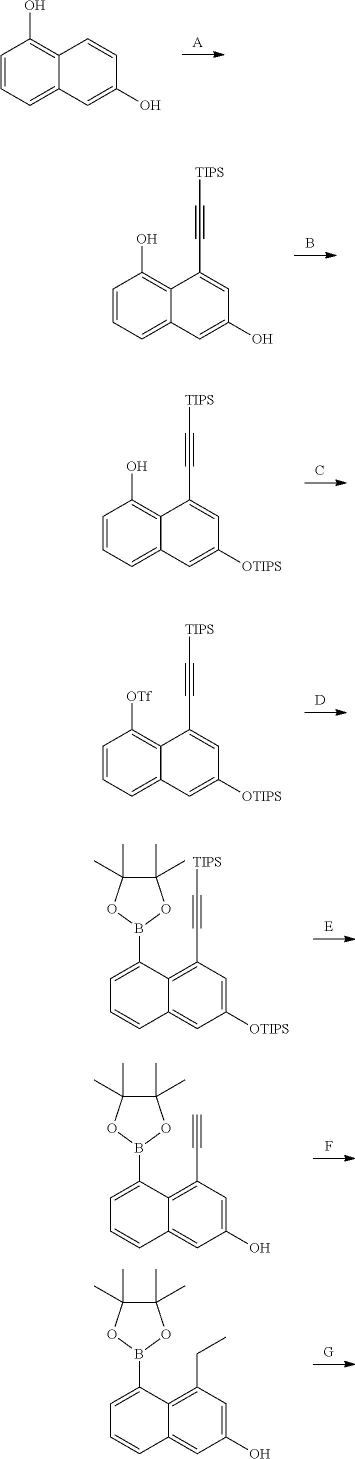

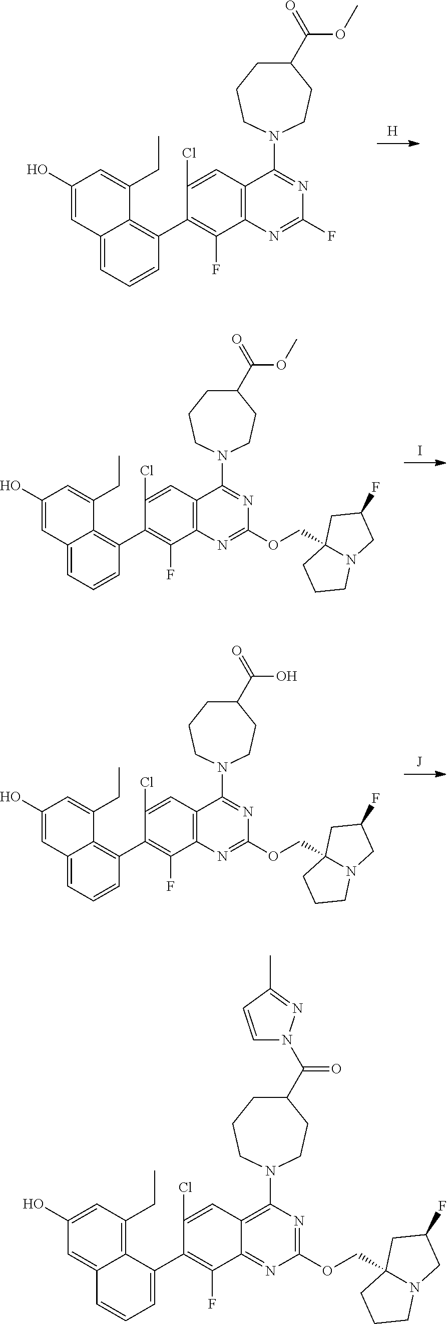

Step A. 8-((triisopropylsilyl)ethynyl)naphthalene-1,6-diol: To a mixture of naphthalene-1,6-diol (5.00 g, 1.0 eq.), (bromoethynyl)triisopropylsilane (9.79 g, 1.2 eq.) and KOAc (6.13 g, 2.0 eq.) in dioxane (30 mL) was added [Ru(p-cymene)Cl₂]₂ (1.91 g, 0.10 eq.). The reaction was degassed and purged with nitrogen 3 times. The reaction was stirred at 110 °C for 2 hours. The mixture was diluted with water (50 mL) and extracted with EtOAc (3×50 mL). The combined organic layers were washed with brine (100 mL), dried over anhydrous sodium sulfate, concentrated, and purified with column chromatography [SiO₂, Petroleum ether/Ethyl acetate=10/1 to 3/1] to afford the title compound. (9.50 g, 84% yield) as yellow solid; ¹H NMR (400 MHz, DMSO-d₆) δ=9.84-9.68 (m, 2H), 7.20-7.14 (m, 2H), 7.13-7.09 (m, 1H), 7.06 (d, J=2.4 Hz, 1H), 6.67-6.66 (m, 1H), 1.16 (br d, J=7.2 Hz, 3H), 1.13 (s, 18H); LCMS (ESI, M+1): m/z=341.3.

Step B. 8-((triisopropylsilyl)ethynyl)-6-((triisopropylsilyl)oxy)naphthalen-1-ol: To a solution of 8-((triisopropylsilyl)ethynyl)naphthalene-1,6-diol (9.50 g, 1.0 eq.) and DIEA (7.21 g, 2.0 eq.) in DCM (100 mL) was added triisopropylsilyl chloride (5.92 g, 1.1 eq.). The reaction was stirred at 0 °C for 0.5 hour. The mixture was diluted with water (100 mL) and extracted with DCM (3×80 mL). The combined organic layers were dried over anhydrous sodium sulfate, concentrated and purified with column chromatography [SiO₂, Petroleum ether/Ethyl acetate=20/1 to 5/1] to afford the title compound (13.5 g, 96% yield) as yellow solid; ¹H NMR (400 MHz, DMSO-d₆) δ=9.92 (br s, 1H), 7.23-7.12 (m, 4H), 6.74 (br d, J=5.6 Hz, 1H), 1.13-1.01 (m, 42H); LCMS (ESI, M+1): m/z=497.3.

Step C. 8-((triisopropylsilyl)ethynyl)-6-((triisopropylsilyl)oxy)naphthalen-1-yl trifluoromethanesulfonate: To a solution of 8-((triisopropylsilyl)ethynyl)-6-((triisopropylsilyl)oxy)naphthalen-1-ol (13.5 g, 1.0 eq.) and DIEA (10.5 g, 3.0 eq.) in DCM (200 mL) was added trifluoromethylsulfonic anhydride (11.5 g, 1.5 eq.). The reaction was stirred at −40 °C for 0.5 hour. The mixture was diluted with water (200 mL) and extracted with DCM (3×100 mL). The combined organic layers were dried over anhydrous sodium sulfate, concentrated and purified with column chromatography [SiO₂, Petroleum ether/Ethyl acetate=10/1 to 5/1] to afford the title compound (15.2 g, 89% yield) as yellow solid; ¹H NMR (400 MHz, DMSO-d₆) δ=8.16-7.95 (m, 1H), 7.71-7.36 (m, 4H), 1.17 (br s, 6H), 1.17-1.10 (m, 36H).

Step D. triisopropyl((8-(4,4,5,5-tetramethyl-1,3,2-dioxaborolan-2-yl)-3-((triisopropylsilyl)oxy)naphthalen-1-yl)ethynyl)silane: To a mixture of 8-((triisopropylsilyl)ethynyl)-6-((triisopropylsilyl)oxy)naphthalen-1-yl trifluoromethanesulfonate (10.0 g, 1.0 eq.), 4,4,5,5-tetramethyl-1,3,2-dioxaborolane (5.09 g, 2.5 eq.) and TEA (4.83 g, 3.0 eq.) in MeCN (100 mL) was added Pd(dppf)Cl₂ (1.16 g, 0.10 eq.). The reaction was degassed and purged with nitrogen 3 times. The reaction was stirred at 90 °C for 6 hours. The mixture was concentrated and purified with column chromatography [SiO₂, Petroleum ether/Ethyl acetate=20/1 to 10/1] to afford the title compound (3.00 g, 28% yield) as yellow solid; LCMS (ESI, M+1): m/z=607.1.

Step E. 4-ethynyl-5-(4,4,5,5-tetramethyl-1,3,2-dioxaborolan-2-yl)naphthalen-2-ol: To a solution of triisopropyl((8-(4,4,5,5-tetramethyl-1,3,2-dioxaborolan-2-yl)-3-((triisopropylsilyl)oxy)naphthalen-1-yl)ethynyl)silane (1.00 g, 1.0 eq.) in DMF (8 mL) was added CsF (1.25 g, 5.0 eq.). The reaction was stirred at 40 °C for 2 hours. The mixture was diluted with water (20 mL) and extracted with EtOAc (3×15 mL). The combined organic layers were washed with brine (30 mL), dried over anhydrous sodium sulfate, concentrated and purified with column chromatography [SiO₂, Petroleum ether/Ethyl acetate=5/1 to 1/1] to afford the title compound (400 mg, 82% yield) as yellow solid; LCMS (ESI, M+1): m/z=295.2.

Step F. 4-ethyl-5-(4,4,5,5-tetramethyl-1,3,2-dioxaborolan-2-yl)naphthalen-2-ol: To a solution of 4-ethynyl-5-(4,4,5,5-tetramethyl-1,3,2-dioxaborolan-2-yl)naphthalen-2-ol (400 mg, 1.0 eq.) in MeOH (5 mL) was added Pd/C (66.7 mg, 10% purity). The reaction was degassed and purged with hydrogen 3 times. The reaction was stirred at 20 °C for 0.1 hours. The mixture was filtered and concentrated to afford the title compound (400 mg, crude) as yellow solid.

Step G. methyl 1-(6-chloro-7-(8-ethyl-6-hydroxynaphthalen-1-yl)-2,8-difluoroquinazolin-4-yl)azepane-4-carboxylate: To a mixture of methyl 1-(7-bromo-6-chloro-2,8-difluoroquinazolin-4-yl)azepane-4-carboxylate (300 mg, 1.0 eq.), 4-ethyl-5-(4,4,5,5-tetramethyl-1,3,2-dioxaborolan-2-yl)naphthalen-2-ol (246.9 mg, 1.2 eq.) and K₃PO₄ (1.5 M, 1.38 mL, 3.0 eq.) in THF (10 mL) was added Sphos Pd G3 (53.7 mg, 0.10 eq.). The reaction was degassed and purged with nitrogen 3 times. The mixture was stirred at 60 °C for 2 hours. The mixture was diluted with water (10 mL) and extracted with EtOAc (3×10 mL). The combined organic layers were washed with brine (20 mL), dried over anhydrous sodium sulfate and concentrated. The mixture was purified with column chromatography [SiO₂, Petroleum ether/Ethyl acetate=1/1 to 0/1] and reversed phase flash chromatography [C18, 0.1% formic acid condition] to afford the title compound (50.0 mg, 13% yield) as white solid; LCMS (ESI, M+1): m/z=526.2.

Step H. methyl 1-(6-chloro-7-(8-ethyl-6-hydroxynaphthalen-1-yl)-8-fluoro-2-(((2R,7aS)-2-fluorotetrahydro-1H-pyrrolizin-7a(5H)-yl)methoxy)quinazolin-4-yl)azepane-4-carboxylate: To a solution of methyl 1-(6-chloro-7-(8-ethyl-6-hydroxynaphthalen-1-yl)-2,8-difluoroquinazolin-4-yl)azepane-4-carboxylate (40.0 mg, 1.0 eq.) and ((2R,7aS)-2-fluorotetrahydro-1H-pyrrolizin-7a(5H)-yl)methanol (14.5 mg, 1.2 eq.) in THF (1 mL) was added t-BuONa (11.0 mg, 1.5 eq.) at 0 °C. The reaction was stirred at 20 °C for 1 hour. The mixture was diluted with water (5 mL) and extracted with EtOAc (3×5 mL). The combined organic layers were washed with brine (10 mL), dried over anhydrous sodium sulfate and concentrated to afford the title compound (50.0 mg, crude) as yellow solid; LCMS (ESI, M+1): m/z=665.4.

Step I. 1-(6-chloro-7-(8-ethyl-6-hydroxynaphthalen-1-yl)-8-fluoro-2-(((2R,7aS)-2-fluorotetrahydro-1H-pyrrolizin-7a(5H)-yl)methoxy)quinazolin-4-yl)azepane-4-carboxylic acid: To a solution of methyl 1-(6-chloro-7-(8-ethyl-6-hydroxynaphthalen-1-yl)-8-fluoro-2-(((2R,7aS)-2-fluorotetrahydro-1H-pyrrolizin-7a(5H)-yl)methoxy)quinazolin-4-yl)azepane-4-carboxylate (50.0 mg, 1.0 eq.) in THF (0.2 mL) and MeOH (0.1 mL) was added a solution of LiOH·H₂O (9.46 mg, 3.0 eq.) in H₂O (0.2 mL). The reaction was stirred at 20 °C for 1 hour. The mixture was acidified with HCl (1 M) and purified with reversed phase flash chromatography [C18, 0.1% formic acid condition] to afford the title compound (30.0 mg, 58% yield) as white solid; LCMS (ESI, M+1): m/z=651.4.

Step J. (1-(6-chloro-7-(8-ethyl-6-hydroxynaphthalen-1-yl)-8-fluoro-2-(((2R,7aS)-2-fluorotetrahydro-1H-pyrrolizin-7a(5H)-yl)methoxy)quinazolin-4-yl)azepan-4-yl)(3-methyl-1H-pyrazol-1-yl)methanone: To a solution of 1-(6-chloro-7-(8-ethyl-6-hydroxynaphthalen-1-yl)-8-fluoro-2-(((2R,7aS)-2-fluorotetrahydro-1H-pyrrolizin-7a(5H)-yl)methoxy)quinazolin-4-yl)azepane-4-carboxylic acid (20.0 mg, 1.0 eq.), 3-methyl-1H-pyrazole (7.57 mg, 3.0 eq.) and DIEA (11.9 mg, 3.0 eq.) in DMF (0.5 mL) was added HATU (23.4 mg, 2.0 eq.). The reaction was stirred at 20 °C for 1 hour. The mixture was filtered. The mixture was purified with reversed phase flash chromatography [C18, neutral condition] and [C18, 0.1% formic acid condition] to afford the title compound (7.17 mg, 29% yield, 0.20HCOOH) as white solid; ¹H NMR (400 MHz, DMSO-d₆) δ=9.84-9.72 (m, 1H), 8.29-8.27 (m, 1H), 8.03-8.02 (m, 1H), 7.83-7.81 (m, 1H), 7.47-7.43 (m, 1H), 7.13-7.11 (m, 1H), 7.03-7.02 (m, 1H), 6.95-6.94 (m, 1H), 6.46-6.45 (m, 1H), 5.33-5.20 (m, 1H), 4.22-4.19 (m, 2H), 4.15-3.95 (m, 3H), 3.93-3.73 (m, 3H), 3.10-3.03 (m, 2H), 2.85-2.76 (m, 1H), 2.37-2.31 (m, 2H), 2.28-2.24 (m, 3H), 2.20-2.08 (m, 4H), 2.05-1.96 (m, 3H), 1.89-1.62 (m, 5H), 0.89 (q, J=7.2 Hz, 3H); LCMS (ESI, M+1): m/z=715.5.

### Example 265

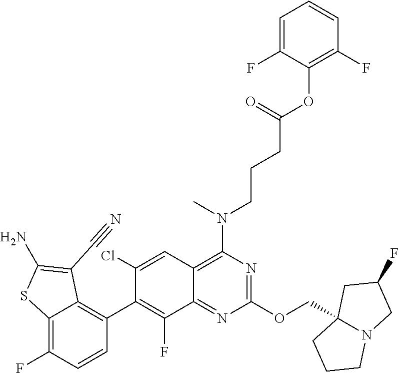 [SMILES: CN(CCCC(=O)Oc1c(F)cccc1F)c1nc(OC[C@@]23CCCN2C[C@H](F)C3)nc2c(F)c(-c3ccc(F)c4sc(N)c(C#N)c34)c(Cl)cc12]

### 2,6-difluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate

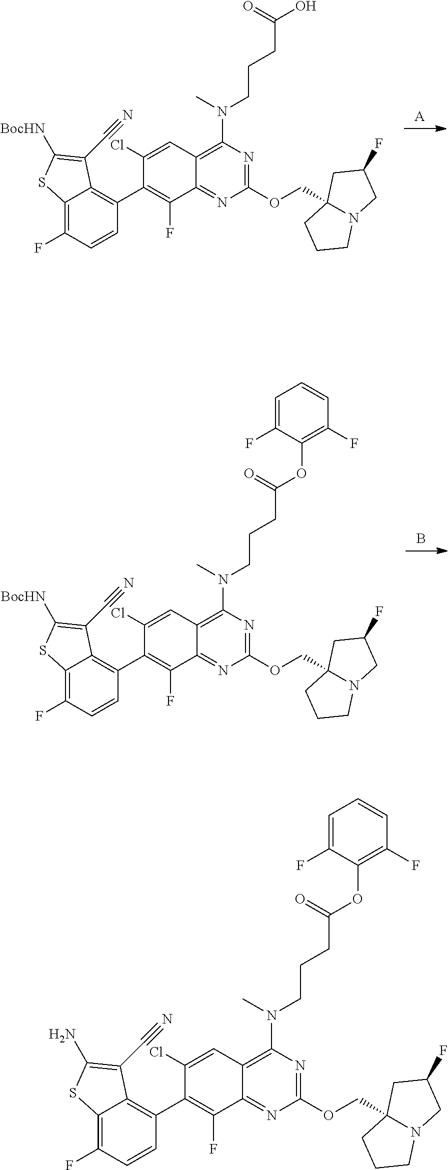

Step A. 2,6-difluorophenyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (100 mg, 1.0 eq.) and 2,6-difluorophenol (26.2 mg, 1.5 eq.) in DMF (1 mL) were added EDCI (38.6 mg, 1.5 eq.) and DMAP (24.6 mg, 1.5 eq.). The reaction was stirred at 60 °C for 2 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 65%-95% B over 15 min] to afford the title compound (65.0 mg, 53% yield) as yellow solid; LCMS (ESI, M+1): m/z=857.2.

Step B. 2,6-difluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate: To a solution of 2,6-difluorophenyl 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate (60.0 mg, 1.0 eq.) in MeCN (3 mL) was added HCl·dioxane (2M, 3 mL). The reaction was stirred at 25 °C for 3 hours. The mixture was concentrated, dissolved in MeOH (2 mL), neutralized with solid NaHCO₃, filtered and purified with prep-HPLC [column: Waters Xbridge 150×25 mm×5 μm; mobile phase: water (NH₄HCO₃)-ACN; gradient: 58%-88% B over 1 min] to afford the title compound (16.6 mg, 30% yield) as white solid; ¹H NMR (400 MHz, CHLOROFORM-d) δ=7.91 (d, J=1.2 Hz, 1H), 7.23-7.15 (m, 2H), 7.03-6.96 (m, 3H), 5.89-5.69 (m, 2H), 5.38-5.15 (m, 1H), 4.35-4.16 (m, 2H), 4.02-3.90 (m, 1H), 3.87-3.77 (m, 1H), 3.48 (s, 3H), 3.39-3.15 (m, 3H), 3.03-2.94 (m, 1H), 2.80 (t, J=6.8 Hz, 2H), 2.31-2.13 (m, 5H), 1.97-1.88 (m, 3H); LCMS (ESI, M+1): m/z=757.1.

### Example 266

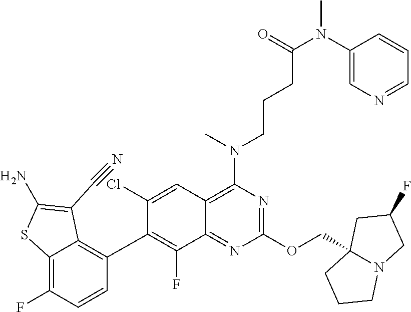 [SMILES: CN(CCCC(=O)N(C)c1cccnc1)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methyl-N-(pyridin-3-yl) butanamide

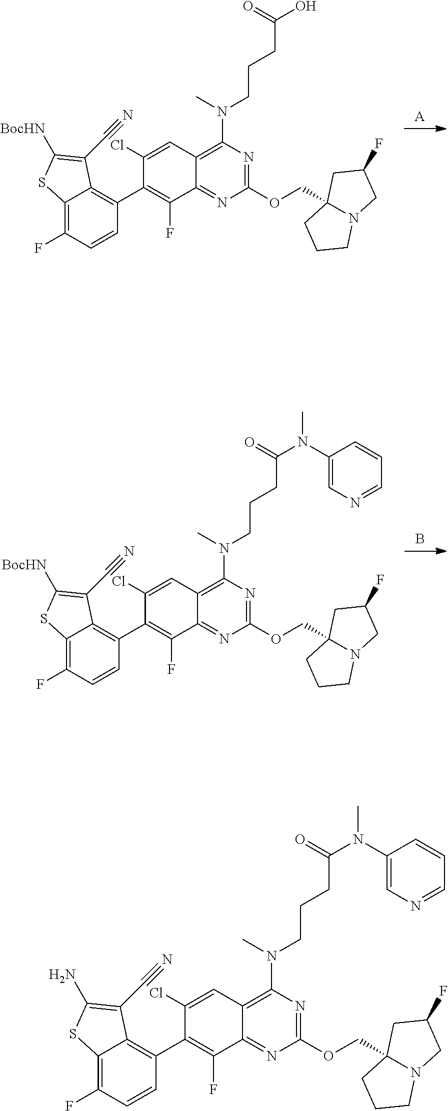

Step A. tert-butyl (4-(6-chloro-8-fluoro-2-(((2R,7aS)-2-fluorotetrahydro-1H-pyrrolizin-7a(5H)-yl)methoxy)-4-(methyl (4-(methyl(pyridin-3-yl)amino)-4-oxobutyl)amino)quinazolin-7-yl)-3-cyano-7-fluorobenzo[b]thiophen-2-yl)carbamate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (80.0 mg, 1.0 eq.) and N-methylpyridin-3-amine (17.4 mg, 1.5 eq.) in 2-MeTHF (2 mL) were added CMPI (41.1 mg, 1.5 eq.) and DIEA (69.4 mg, 5.0 eq.). The reaction was stirred at 40 °C for 1 hour. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Phenomenex luna C18 150×25 mm×10 μm; mobile phase: water (FA)-ACN; gradient: 28%-58% B over 9 min] to afford the title compound (40.0 mg, 44% yield) as white solid; LCMS (ESI, M+1): m/z=835.3.

Step B. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methyl-N-(pyridin-3-yl) butanamide: To a solution of tert-butyl (4-(6-chloro-8-fluoro-2-(((2R,7aS)-2-fluorotetrahydro-1H-pyrrolizin-7a(5H)-yl)methoxy)-4-(methyl (4-(methyl(pyridin-3-yl)amino)-4-oxobutyl)amino)quinazolin-7-yl)-3-cyano-7-fluorobenzo[b]thiophen-2-yl)carbamate (40.0 mg, 1.0 eq.) in CH₃CN (2 mL) was added HCl·dioxane (2M, 2 mL). The reaction was stirred at 25 °C for 2 hours. The mixture was concentrated, dissolved in MeOH (2 mL), neutralized with solid NaHCO₃ and filtered. The filtrate was concentrated and purified with prep-HPLC [column: Phenomenex luna C18 150×25 mm×10 μm; mobile phase: water (FA)-ACN; gradient: 8%-48% B over 10 min] to afford the title compound (19.7 mg, 55% yield, 0.1HCOOH) as white solid; ¹H NMR (400 MHz, CHLOROFORM-d) δ=8.60-8.42 (m, 2H), 7.82 (br s, 1H), 7.58-7.42 (m, 1H), 7.36 (br s, 1H), 7.19-7.13 (m, 1H), 6.95 (t, J=8.8 Hz, 1H), 6.64-6.26 (m, 2H), 5.47-5.28 (m, 1H), 4.53-4.33 (m, 2H), 3.79-3.57 (m, 4H), 3.35 (br s, 4H), 3.26 (br s, 3H), 3.11 (br s, 1H), 2.53-2.30 (m, 3H), 2.19-2.00 (m, 7H); LCMS (ESI, M+1): m/z=735.2.

### Example 267

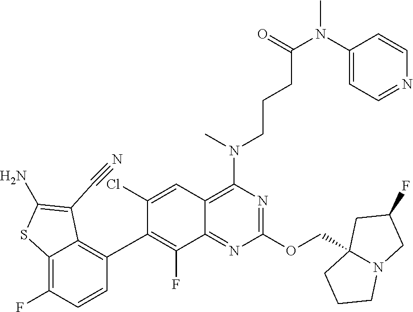 [SMILES: CN(CCCC(=O)N(C)c1ccncc1)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methyl-N-(pyridin-4-yl) butanamide

Synthesized according to the method used for Example 265. The title compound was obtained as yellow solid; 1HNMR (400 MHz, CHLOROFORM-d) δ=8.63 (d, J=5.6 Hz, 2H), 7.85 (d, J=0.8 Hz, 1H), 7.25-7.17 (m, 1H), 7.10 (d, J=5.6 Hz, 2H), 7.02 (t, J=9.2 Hz, 1H), 6.05-5.81 (m, 2H), 5.54-5.07 (m, 1H), 4.38-4.09 (m, 2H), 3.89-3.64 (m, 2H), 3.48-3.42 (m, 1H), 3.39 (s, 3H), 3.36-3.30 (m, 1H), 3.29 (s, 3H), 3.26-3.20 (m, 1H), 3.10-2.94 (m, 1H), 2.38-2.33 (m, 1H), 2.25-2.10 (m, 4H), 2.06-1.87 (m, 4H); LCMS (ESI, M+1): m/z=735.2.

### Example 268

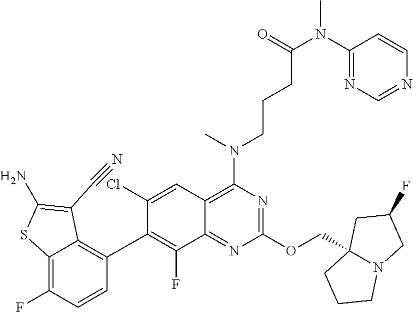 [SMILES: CN(CCCC(=O)N(C)c1ccncn1)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-methyl-N-(pyrimidin-4-yl) butanamide

Synthesized according to the method used for Example 265. The title compound was obtained as yellow solid; ¹HNMR (400 MHz, METHANOL-d₄) δ=8.92 (s, 1H), 8.59 (d, J=6.0 Hz, 1H), 8.15 (s, 1H), 7.88-7.84 (m, 1H), 7.26-7.19 (m, 1H), 7.05 (t, J=9.2 Hz, 1H), 5.58-5.29 (m, 1H), 4.61-4.36 (m, 2H), 4.05-3.88 (m, 2H), 3.81-3.59 (m, 3H), 3.55 (s, 3H), 3.47 (s, 3H), 3.29-3.24 (m, 1H), 2.86 (t, J=6.8 Hz, 2H), 2.58-2.39 (m, 2H), 2.33-2.15 (m, 5H), 2.08-1.94 (m, 1H); LCMS (ESI, M+1): m/z=736.2.

### Example 269

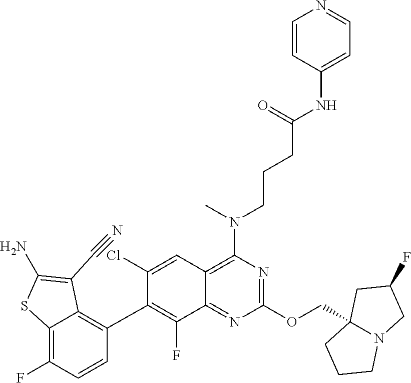 [SMILES: CN(CCCC(=O)Nc1ccncc1)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(pyridin-4-yl) butanamide

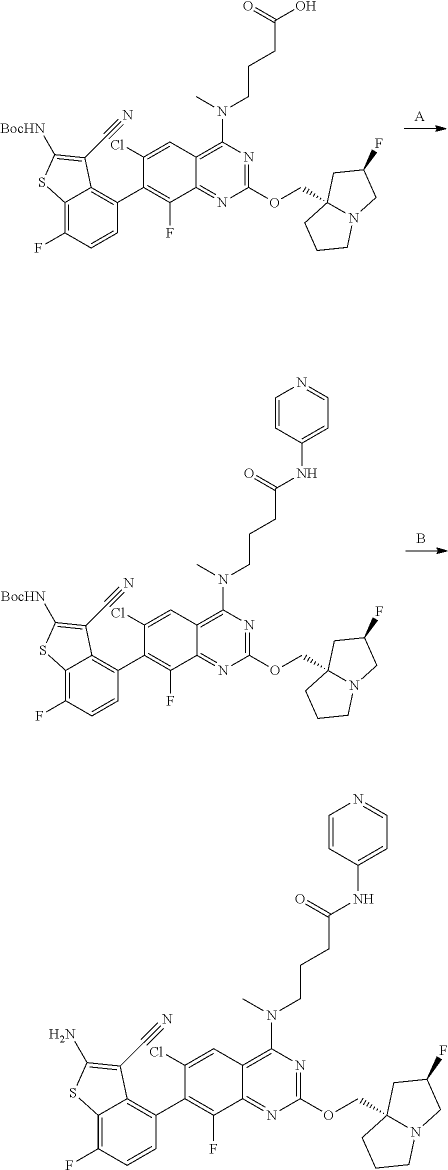

Step A. tert-butyl (4-(6-chloro-8-fluoro-2-(((2R,7aS)-2-fluorotetrahydro-1H-pyrrolizin-7a(5H)-yl)methoxy)-4-(methyl (4-oxo-4-(pyridin-4-ylamino)butyl)amino)quinazolin-7-yl)-3-cyano-7-fluorobenzo[b]thiophen-2-yl)carbamate: To a solution of 4-((7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoic acid (200 mg, 1.0 eq.) in 2-methyltetrahydrofuran (5 mL) were added DIEA (173 mg, 5.0 eq.), pyridin-4-amine (126 mg, 5.0 eq.) and CMPI (343 mg, 5.0 eq.). The reaction was stirred at 40 °C for 2 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Phenomenex luna C18 150×40 mm×15 μm; mobile phase: [water (FA)-ACN]; gradient: 18%-48% B over 15 min] to afford the title compound (70.0 mg, 46% yield) as white solid; LCMS (ESI, M+1): m/z=821.4.

Step B. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(pyridin-4-yl) butanamide: To a solution of tert-butyl (4-(6-chloro-8-fluoro-2-(((2R,7aS)-2-fluorotetrahydro-1H-pyrrolizin-7a(5H)-yl)methoxy)-4-(methyl (4-oxo-4-(pyridin-4-ylamino)butyl)amino)quinazolin-7-yl)-3-cyano-7-fluorobenzo[b]thiophen-2-yl)carbamate (103 mg, 1.0 eq.) in DCM (3 mL) was added TFA (1 mL). The reaction was stirred at 20 °C for 1 hour. The mixture was neutralized with saturated NaHCO₃ solution at 0 °C and extracted with DCM (3×5 mL). The combined organic layers were washed with brine (10 mL), dried over anhydrous sodium sulfate, concentrated and purified with prep-HPLC [column: Waters Xbridge 150×25 mm×5 μm; mobile phase: [water (ammonia hydroxide v/v)-ACN]; gradient: 33%-63% B over 10 min] to afford the title compound (22.4 mg, 24% yield) as white solid; ¹H NMR (400 MHz, CHLOROFORM-d) δ=9.14 (br s, 1H), 8.37 (br d, J=6.0 Hz, 2H), 7.81 (br s, 1H), 7.43 (br d, J=5.2 Hz, 2H), 7.21-7.15 (m, 1H), 7.01 (t, J=8.8 Hz, 1H), 6.12 (br d, J=19.2 Hz, 2H), 5.33-5.09 (m, 1H), 4.26-4.09 (m, 2H), 4.08-3.95 (m, 1H), 3.84-3.70 (m, 1H), 3.35 (d, J=3.6 Hz, 3H), 3.32-3.13 (m, 3H), 3.03-2.94 (m, 1H), 2.46-2.33 (m, 2H), 2.27-1.91 (m, 8H); LCMS (ESI, M+1): m/z=721.2.

### Example 270

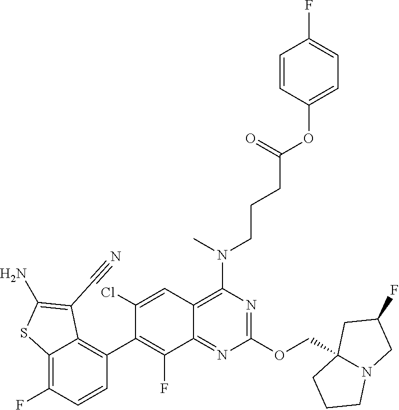 [SMILES: CN(CCCC(=O)Oc1ccc(F)cc1)c1nc(OC[C@@]23CCCN2C[C@H](F)C3)nc2c(F)c(-c3ccc(F)c4sc(N)c(C#N)c34)c(Cl)cc12]

### 4-fluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate

Synthesized according to the method used for Example 265. The title compound was obtained as white solid; ¹H NMR (400 MHz, CHLOROFORM-d) δ=7.89 (d, J=1.6 Hz, 1H), 7.19 (dd, J=4.8, 8.4 Hz, 1H), 7.08-6.99 (m, 5H), 5.73 (br d, J=15.2 Hz, 2H), 5.34-5.16 (m, 1H), 4.31-4.22 (m, 1H), 4.21-4.10 (m, 1H), 3.99-3.90 (m, 1H), 3.88-3.79 (m, 1H), 3.46 (s, 3H), 3.31-3.13 (m, 3H), 3.02-2.93 (m, 1H), 2.69 (t, J=7.2 Hz, 2H), 2.28-2.17 (m, 4H), 2.16-2.10 (m, 1H), 1.96-1.84 (m, 3H); LCMS (ESI, M+1): m/z=739.2.

### Example 271

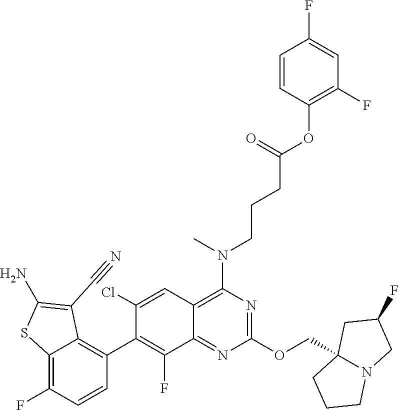 [SMILES: CN(CCCC(=O)Oc1ccc(F)cc1F)c1nc(OC[C@@]23CCCN2C[C@H](F)C3)nc2c(F)c(-c3ccc(F)c4sc(N)c(C#N)c34)c(Cl)cc12]

### 2,4-difluorophenyl 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoate

Synthesized according to the method used for Example 266. The title compound was obtained as white solid; ¹H NMR (400 MHz, CHLOROFORM-d) δ=7.89 (s, 1H), 7.19-7.15 (m, 1H), 7.08 (dt, J=5.6, 8.8 Hz, 1H), 6.98-6.85 (m, 3H), 6.16-6.05 (m, 2H), 5.34-5.21 (m, 1H), 4.33-4.25 (m, 2H), 3.97-3.87 (m, 1H), 3.83-3.75 (m, 1H), 3.44 (s, 3H), 3.40 (br d, J=4.8 Hz, 1H), 3.31-3.18 (m, 2H), 3.04-2.98 (m, 1H), 2.73 (dt, J=3.4, 7.0 Hz, 2H), 2.38-2.29 (m, 1H), 2.24-2.12 (m, 4H), 1.98-1.93 (m, 3H); LCMS (ESI, M+1): m/z=757.2.

### Example 272

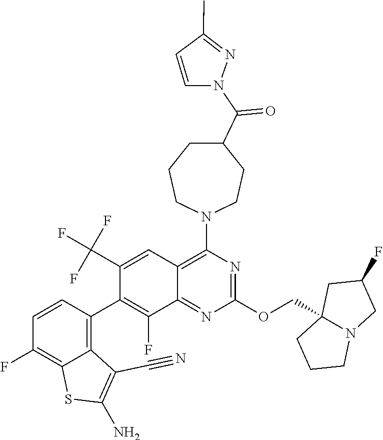 [SMILES: Cc1ccn(C(=O)C2CCCN(c3nc(OC[C@@]45CCCN4C[C@H](F)C5)nc4c(F)c(-c5ccc(F)c6sc(N)c(C#N)c56)c(C(F)(F)F)cc34)CC2)n1]

### 2-amino-7-fluoro-4-(8-fluoro-2-(((2R,7aS)-2-fluorotetrahydro-1H-pyrrolizin-7a(5H)-yl)methoxy)-4-(4-(3-methyl-1H-pyrazole-1-carbonyl)azepan-1-yl)-6-(trifluoromethyl)quinazolin-7-yl)benzo[b]thiophene-3-carbonitrile

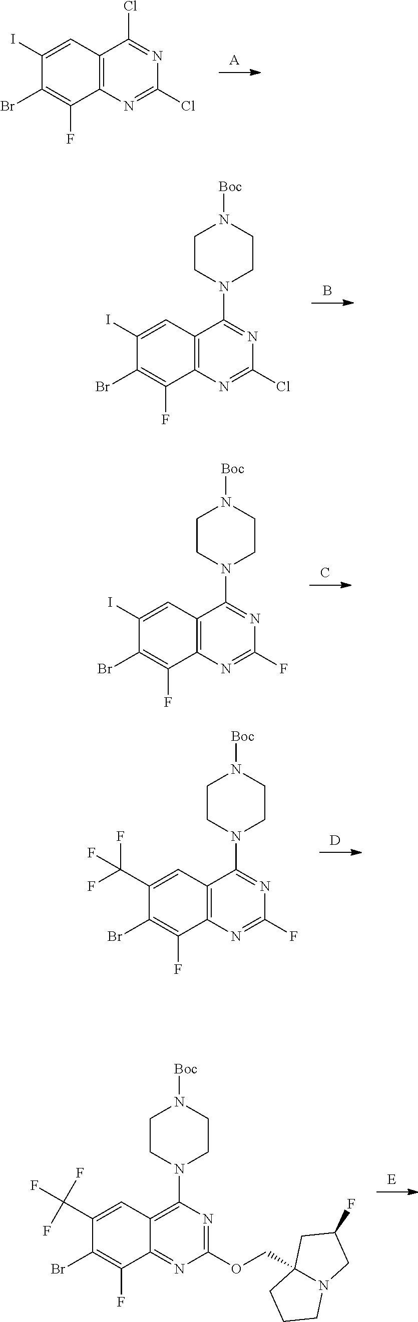

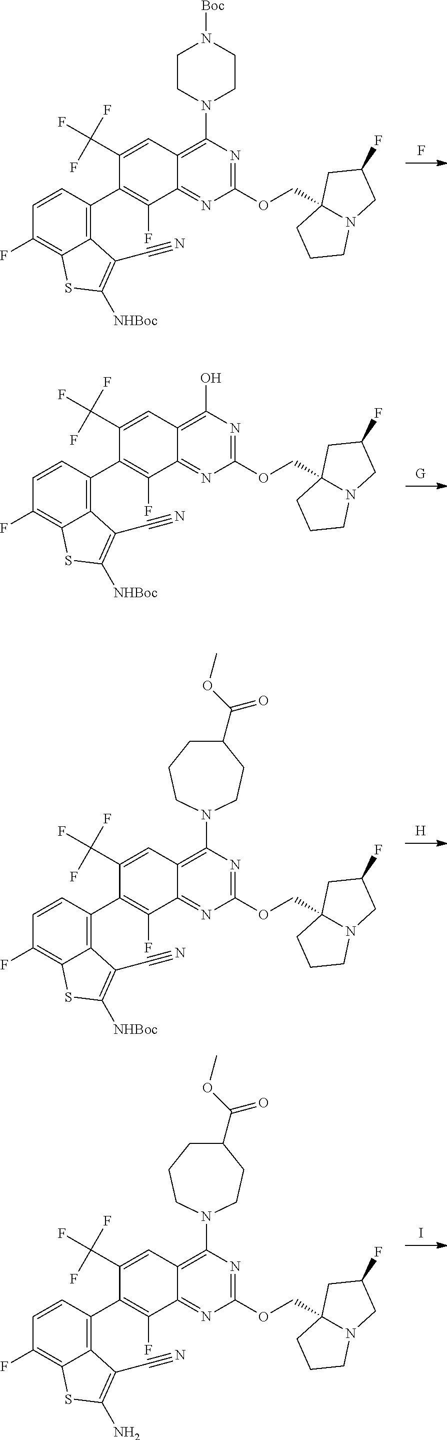

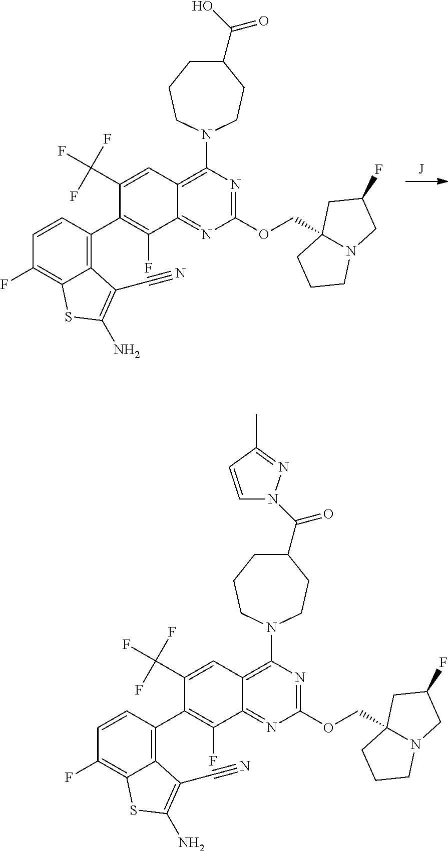

Step A. tert-butyl 4-(7-bromo-2-chloro-8-fluoro-6-iodoquinazolin-4-yl) piperazine-1-carboxylate: To a solution 7-bromo-2,4-dichloro-8-fluoro-6-iodo-quinazoline (10.4 g, 1.0 eq.) and DIPEA (9.6 g, 3.0 eq.) in DCM (140 mL) was added tert-butyl piperazine-1-carboxylate (4.6 g, 1.0 eq.) at −40 °C under N₂ atmosphere. The reaction was stirred at −40 °C for 0.5 hour. The mixture was quenched with H₂O (200 mL) at −40 °C and extracted with DCM (3×150 mL). The combined organic layers were washed with 0.1N HCl (2×50 mL), saturated NaHCO₃ aqueous solution (50 mL) and brine (50 mL). The solution was dried over anhydrous sodium sulfate, concentrated, and triturated with ACN (25 mL) to afford the title compound (12.7 g, 88% yield) as yellow solid; LCMS (ESI, M+1, M+3): m/z=571.0, 573.0.

Step B. tert-butyl 4-(7-bromo-2,8-difluoro-6-iodoquinazolin-4-yl) piperazine-1-carboxylate: To a solution of tert-butyl 4-(7-bromo-2-chloro-8-fluoro-6-iodoquinazolin-4-yl) piperazine-1-carboxylate (6.35 g, 1 eq.) and 18-crown-6 ether (294 mg, 0.1 eq.) in DMSO (75 mL) was added KF (6.45 g, 10.0 eq.). The reaction was stirred at 130 °C for 5 hours. The mixture was poured into H₂O (0.8 L). The mixture was filtered, and the filter cake was washed with H₂O (100 mL). The solid was dissolved in DCM (500 mL). The organic layer was washed with brine (50 mL), dried over anhydrous sodium sulfate, concentrated, and triturated with ACN (20 mL) to afford the title compound (3.9 g, 63% yield) as light yellow solid; LCMS (ESI, M+1, M+3): m/z=554.9, 556.9

Step C. tert-butyl 4-(7-bromo-2,8-difluoro-6-(trifluoromethyl)quinazolin-4-yl) piperazine-1-carboxylate: To a solution of tert-butyl 4-(7-bromo-2,8-difluoro-6-iodoquinazolin-4-yl) piperazine-1-carboxylate (2.9 g, 1.0 eq.) and methyl 2,2-difluoro-2-(fluorosulfonyl)acetate (2.51 g, 2.5 eq.) in DMAc (60 mL) was added CuI (2.98 g, 3.0 eq.). The reaction was degassed and purged with nitrogen 3 times. The reaction was stirred at 90 °C for 24 hours. The mixture was poured into H₂O (1 L) and NH₃·H₂O (25% w/w, 20 mL). The mixture was filtered and the solid was washed with H₂O (100 mL). The solid was dissolved in DCM (400 mL). The organic layer was washed with brine (50 mL), dried over anhydrous sodium sulfate, concentrated, and purified with column chromatography [SiO₂, PE/EtOAc/DCM=8/1/1 to 6/1/1] to afford the title compound (1.6 g, 61% yield) as light yellow solid; LCMS (ESI, M+1, M+3): m/z=497.0, 499.0

Step D. tert-butyl 4-(7-bromo-8-fluoro-2-(((2R,7aS)-2-fluorotetrahydro-1H-pyrrolizin-7a(5H)-yl)methoxy)-6-(trifluoromethyl)quinazolin-4-yl) piperazine-1-carboxylate: To a solution of tert-butyl 4-(7-bromo-2,8-difluoro-6-(trifluoromethyl)quinazolin-4-yl) piperazine-1-carboxylate (1.1 g, 1.0 eq.) and ((2R,7aS)-2-fluorotetrahydro-1H-pyrrolizin-7a(5H)-yl)methanol (369 mg, 1.05 eq.) in THF (15 mL) was added t-BuONa (276 mg, 1.3 eq.) at 0 °C. The reaction was stirred at 0 °C for 1 hour. The mixture was diluted with H₂O (100 mL) and extracted with EtOAc (3×30 mL). The combined organic layers were washed with brine (50 mL), dried over anhydrous sodium sulfate and concentrated to afford the title compound (1.4 g, 99% yield) as light yellow solid; LCMS (ESI, M+1, M+3): m/z=636.1, 638.1.

Step E. tert-butyl 4-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl) piperazine-1-carboxylate: To a solution of tert-butyl 4-(7-bromo-8-fluoro-2-(((2R,7aS)-2-fluorotetrahydro-1H-pyrrolizin-7a(5H)-yl)methoxy)-6-(trifluoromethyl)quinazolin-4-yl) piperazine-1-carboxylate (650 mg, 1.0 eq.) and tert-butyl (3-cyano-4-(5,5-dimethyl-1,3,2-dioxaborinan-2-yl)-7-fluorobenzo[b]thiophen-2-yl)carbamate (1.03 g, 2.5 eq.) in dioxane (13 mL) were added Cs₂CO₃ (832 mg, 2.5 eq.) and DPEPhosPdCl₂ (146 mg, 0.2 eq.). The reaction was degassed and purged with nitrogen 3 times. The reaction was stirred at 90 °C for 9.5 hours. The mixture was diluted with H₂O (30 mL) and extracted with DCM (4×15 mL). The combined organic layers were washed with brine (15 mL), dried over anhydrous sodium sulfate, concentrated and purified with column chromatography [Al₂O₃, PE/EtOAc=2/1 to 0/1] and reversed phase flash chromatography [water (FA, 0.1%)/acetonitrile=1/1] to afford the title compound (0.23 g, 26% yield) as brown solid; LCMS (ESI, M+1): m/z=848.2.

Step F. tert-butyl (3-cyano-7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[b]thiophen-2-yl)carbamate: To a solution of tert-butyl 4-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl) piperazine-1-carboxylate (460 mg, 1.0 eq.) in EtOH (7 mL) was added NaOH (2 M in H₂O, 1.4 mL, 5.2 eq.). The reaction was stirred at 50 °C for 12 hours. The mixture was neutralized with 3M HCl at 0 °C, concentrated and purified with reversed phase flash chromatography [water (FA, 0.1%)/acetonitrile=1/1] to afford the title compound (255 mg, 69% yield) as light yellow solid; LCMS (ESI, M+1): m/z=680.1.

Step G. methyl 1-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-4-carboxylate: To a solution of methyl azepane-4-carboxylate hydrogen chloride (261 mg, 3.0 eq.) and TEA (454 mg 10.0 eq.) in DMSO (3 mL) were added tert-butyl (3-cyano-7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[b]thiophen-2-yl)carbamate (305 mg, 1.0 eq.) and PyBOP (467.08 mg, 2.0 eq.). The reaction was stirred at 40 °C for 3 hours. The mixture was filtered and purified with reversed phase flash chromatography [water (NH₃·H₂O, 0.1%)/acetonitrile=7/3] to afford the title compound (320 mg, 86% yield) as light yellow solid; LCMS (ESI, M+1): m/z=819.2.

Step H. methyl 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-4-carboxylate: To a solution of methyl 1-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-4-carboxylate (160 mg, 1.0 eq.) in DCM (1 mL) was added TFA (1.5 mL) dropwise at 0 °C. The reaction was stirred at 20 °C for 0.5 hour. The mixture was concentrated. The residue was partitioned between EtOAc (10 mL) and saturated Na₂CO₃ aqueous solution (10 mL) at 0 °C. The mixture was extracted with EtOAc (4×10 mL). The combined organic layers were washed with brine (10 mL), dried over anhydrous sodium sulfate and concentrated to afford the title compound (280 mg, 99% yield) as light yellow solid; LCMS (ESI, M+1): m/z=719.1.

Step I. 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-4-carboxylic acid: To a solution of methyl 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-4-carboxylate (150 mg, 1.0 eq.) in MeOH (1 mL) and THF (0.5 mL) was added LiOH·H₂O (1.5 M in H₂O, 417 μL, 3.0 eq.). The reaction was stirred at 20 °C for 0.5 hour. The mixture was neutralized with 3M HCl at 0 °C, concentrated and purified with reversed phase flash chromatography [water (FA, 0.1%)/acetonitrile=7/3] to afford the title compound (82.5 mg, 56% yield) as white solid; LCMS (ESI, M+1): m/z=705.2.

Step J. 2-amino-7-fluoro-4-(8-fluoro-2-(((2R,7aS)-2-fluorotetrahydro-1H-pyrrolizin-7a(5H)-yl)methoxy)-4-(4-(3-methyl-1H-pyrazole-1-carbonyl)azepan-1-yl)-6-(trifluoromethyl)quinazolin-7-yl)benzo[b]thiophene-3-carbonitrile: To a solution of 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)azepane-4-carboxylic acid (50.0 mg, 1.0 eq.) and 3-methyl-1H-pyrazole (20.4 mg, 3.5 eq.) in DMAc (0.75 mL) were added DIPEA (36.7 mg, 4.0 eq.) and HATU (45.9 mg, 1.7 eq.). The reaction was stirred at 20 °C for 3 hours. The mixture was filtered and purified with reversed phase flash chromatography [water/acetonitrile=3/7] to afford the title compound (21.5 mg, 39% yield) as white solid; ¹H NMR (400 MHz, DMSO-d₆) δ=8.28 (d, J=2.8 Hz, 1H), 8.24 (s, 1H), 8.07 (br s, 2H), 7.27-7.20 (m, 1H), 7.16-7.10 (m, 1H), 6.46 (d, J=2.8 Hz, 1H), 5.26 (d, J=54.0 Hz, 1H), 4.30-3.73 (m, 7H), 3.14-2.93 (m, 3H), 2.87-2.76 (m, 1H), 2.33-1.95 (m, 11H), 1.86-1.61 (m, 4H); ¹⁹F NMR (376 MHz, DMSO) δ=−57.2, −116,−123,−172; LCMS (ESI, M+1): m/z=769.2.

### Example 273

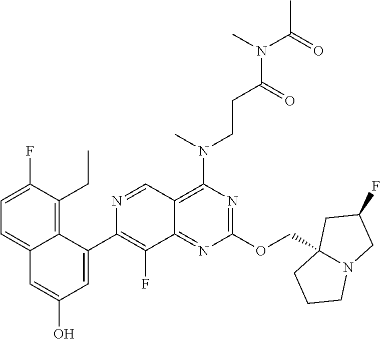 [SMILES: CCc1c(F)ccc2cc(O)cc(-c3ncc4c(N(C)CCC(=O)N(C)C(C)=O)nc(OC[C@@]56CCCN5C[C@H](F)C6)nc4c3F)c12]

### N-acetyl-3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpropanamide

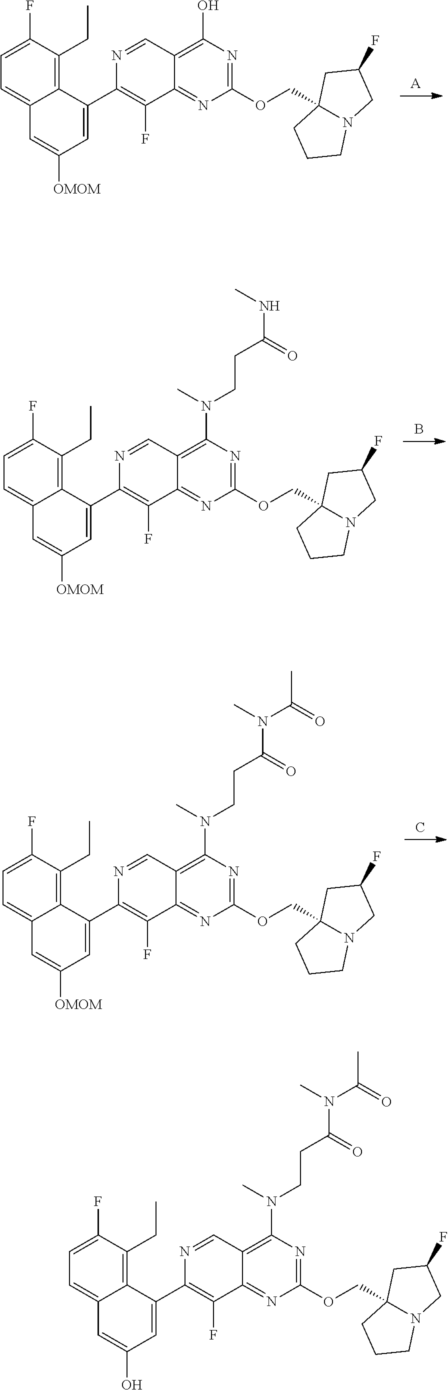

Step A. 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpropanamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (400 mg, 1.0 eq.) and N-methyl-3-(methylamino) propanamide (126 mg, 1.5 eq.) in DMF (4 mL) were added PyBOP (375 mg, 1.0 eq.) and DIEA (280 mg, 3.0 eq.). The reaction was stirred at 30 °C for 2 hours. The mixture was diluted with water (2 mL) and extracted with ethyl acetate (3×2 mL). The organic layers were dried over anhydrous sodium sulfate, concentrated, and purified with reversed phase flash chromatography (0.1% formic acid condition) to afford the title compound (420 mg, 79% yield) as yellow oil.

Step B. N-acetyl-3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpropanamide: To a solution of 3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpropanamide (220 mg, 1.0 eq.) in THF (2.5 mL) was added NaH (40.4 mg, 3.0 eq.) at 0 °C. The reaction was stirred at 25 °C for 0.5 hour. To the above mixture was added acetyl chloride (47.6 mg, 1.8 eq.) dropwise at 0 °C. The reaction was stirred at 40 °C for 2 hours under nitrogen atmosphere. The mixture was quenched with ice-water (1 mL) and extracted with ethyl acetate (2×1 mL). The combined organic layers were washed with brine (1 mL), dried over anhydrous sodium sulfate, concentrated and purified with reversed phase flash chromatography (0.1% formic acid condition) to afford the title compound (200 mg, 77% yield) as yellow oil; LCMS (ESI, M+1): m/z=695.4.

Step C. N-acetyl-3-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpropanamide: To a solution of N-acetyl-3-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpropanamide (100 mg, 1.0 eq.) in DCM (1 mL) was added TFA (1.54 g, 93 eq.) at 0 °C. The reaction was stirred at 25 °C for 2 hours. The mixture was concentrated, basified with saturated NaHCO₃ solution (6 mL) and extracted with ethyl acetate (3×5 mL). The combined organic layers were dried over anhydrous sodium sulfate, concentrated and purified with prep-HPLC [Waters Xbridge 150×25 mm×5 μm; A: water (NH₄HCO₃), B: ACN, B %: 40%-70% over 9 min] to afford the title compound (4.21 mg, 4% yield) as white solid; ¹H NMR (400 MHz, CHLOROFORM-d₃) δ=9.09 (br s, 1H), 7.58-7.48 (m, 1H), 7.22-7.13 (m, 2H), 7.07-6.92 (m, 1H), 5.45-5.19 (m, 1H), 4.48-4.22 (m, 2H), 4.12 (br t, J=5.6 Hz, 2H), 3.59-3.47 (m, 3H), 3.44-3.28 (m, 4H), 3.25 (s, 3H), 3.21 (br d, J=2.4 Hz, 1H), 3.08-2.97 (m, 1H), 2.56-2.45 (m, 1H), 2.39 (s, 3H), 2.33-2.07 (m, 4H), 2.05-1.88 (m, 3H), 0.88-0.75 (m, 3H); LCMS (ESI, M+1): m/z=651.3.

### Example 274

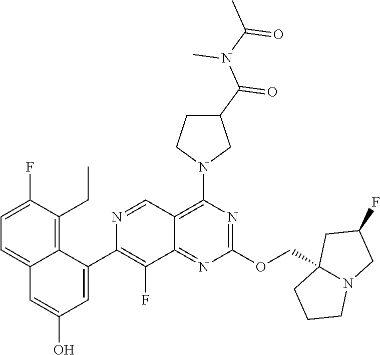 [SMILES: CCc1c(F)ccc2cc(O)cc(-c3ncc4c(N5CCC(C(=O)N(C)C(C)=O)C5)nc(OC[C@@]56CCCN5C[C@H](F)C6)nc4c3F)c12]

### N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpyrrolidine-3-carboxamide

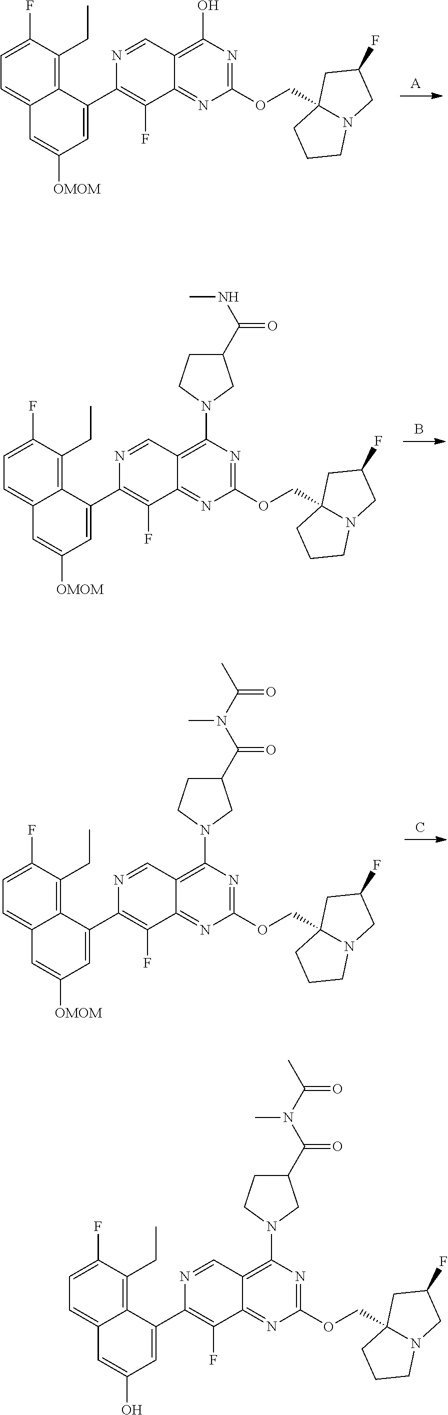

Step A. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpyrrolidine-3-carboxamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (300 mg, 1.0 eq.) and N-methylpyrrolidine-3-carboxamide hydrochloride (133 mg, 1.5 eq.) in DMF (3 mL) were added PyBOP (563 mg, 2.0 eq.) and DIEA (349 mg, 5.0 eq.). The reaction was stirred at 25 °C for 1 hour. The mixture was filtered, and the filtrate was purified with reversed phase flash chromatography [C18, 0.1% NH₃·H₂O condition] to afford the title compound (309 mg, 85% yield) as white solid; LCMS (ESI, M+1): m/z=665.3.

Step B. N-acetyl-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpyrrolidine-3-carboxamide: To a solution of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpyrrolidine-3-carboxamide (100 mg, 1.0 eq.) in THF (0.5 mL) was added NaH (10.8 mg, 60% purity, 1.8 eq.) at 0 °C. The reaction was stirred at 25 °C for 0.5 hour. To the above mixture was added a solution of acetyl chloride (17.7 mg, 1.5 eq.) in THF (0.5 mL) at 0 °C. The reaction was stirred at 40 °C for 11.5 hours. The mixture was concentrated. The residue was quenched with saturated NaHCO₃ solution (5 mL) at 0 °C and extracted with ethyl acetate (2×5 mL). The combined organic layers were dried over anhydrous sodium sulfate and concentrated to afford the title compound (100 mg, crude) as yellow oil.

Step C. N-acetyl-1-(7-(8-ethyl-7-fluoro-3-hydroxynaphthalen-1-yl)-8-fluoro-2-yl)-N-methylpyrrolidine-3-carboxamide: To a solution of N-acetyl-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylpyrrolidine-3-carboxamide (100 mg, 1.0 eq.) in DCM (1 mL) was added TFA (1.54 g, 95 eq.). The reaction was stirred at 25 °C for 15 minutes. The mixture was concentrated, basified with saturated NaHCO₃ solution (6 mL) and extracted with ethyl acetate (3×5 mL). The combined organic layers were dried over anhydrous sodium sulfate, concentrated and purified with prep-HPLC [Waters Xbridge 150×25 mm×5 μm; A: water (NH₄HCO₃), B: ACN, B %: 40%-70% over 9 min] and prep-HPLC [Phenomenex Luna C18 150×25 mm×10 μm; A: water (FA), B: ACN, B %: 18%-48% over 10 min] to afford the title compound (12.6 mg, 13% yield) as white solid; ¹H NMR (400 MHz, CHLOROFORM-d₃) δ=9.17-9.07 (m, 1H), 7.58-7.48 (m, 1H), 7.21-7.11 (m, 2H), 7.07-6.89 (m, 1H), 5.44-5.18 (m, 1H), 4.49-4.25 (m, 2H), 4.23-3.91 (m, 5H), 3.57-3.29 (m, 2H), 3.28-3.23 (m, 3H), 3.21-2.95 (m, 2H), 2.44-2.36 (m, 3H), 2.35-2.08 (m, 7H), 2.05-1.91 (m, 3H), 0.81 (t, J=7.4 Hz, 3H); LCMS (ESI, M+1): m/z=663.1.

### Example 275

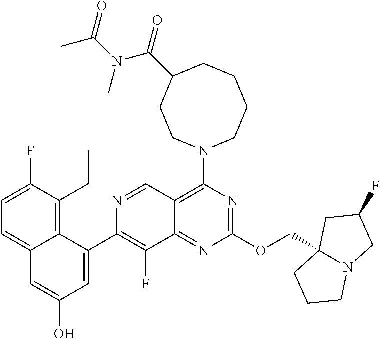 [SMILES: CCc1c(F)ccc2cc(O)cc(-c3ncc4c(N5CCCCC(C(=O)N(C)C(C)=O)CC5)nc(OC[C@@]56CCCN5C[C@H](F)C6)nc4c3F)c12]

### N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazocane-4-carboxamide

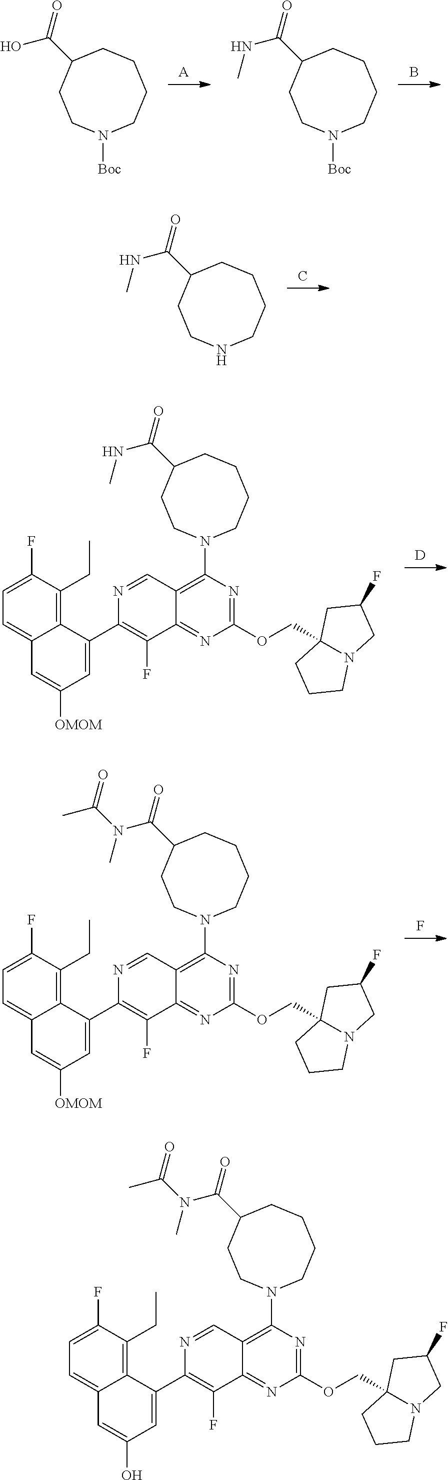

Step A. tert-butyl 4-(methylcarbamoyl)azocane-1-carboxylate: To a solution of 1-(tert-butoxycarbonyl)azocane-4-carboxylic acid (400 mg, 1.0 eq.) and methanamine hydrochloride (210 mg, 2.0 eq.) in DMF (5 mL) were added DIEA (1.61 g, 8.0 eq.) and HATU (1.18 g, 2.0 eq.). The reaction was stirred at 20 °C for 1 hour. The mixture was diluted with water (6 mL) and extracted with ethyl acetate (3×5 mL). The combined organic layers were washed with brine (2×10 mL), dried over anhydrous sodium sulfate and concentrated to afford the title compound (470 mg, crude) as yellow oil.

Step B. N-methylazocane-4-carboxamide: To a solution of tert-butyl 4-(methylcarbamoyl)azocane-1-carboxylate (470 mg, 1.0 eq.) in ACN (0.5 mL) was added HCl·dioxane (2 M, 2.0 mL, 2.3 eq.). The reaction was stirred at 15 °C for 0.5 hour. The mixture was concentrated to afford the title compound (300 mg, HCl) as yellow oil.

Step C. 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazocane-4-carboxamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (400 mg, 1.0 eq.) and N-methylazocane-4-carboxamide (298 mg, 2.0 equiv, HCl) in DMF (5 mL) were added PyBOP (751 mg, 2.0 eq.) and DIEA (466 mg, 5.0 eq.). The reaction was stirred at 30 °C for 1 hour. The mixture was filtered and purified with reversed phase flash chromatography [water (0.1% NH₃·H₂O)/acetonitrile] to afford the title compound (350 mg, 68% yield) as yellow solid; ¹H NMR (400 MHz, CHLOROFORM-d₃) δ=9.10 (s, 1H), 7.68 (dd, J=6.0, 9.2 Hz, 1H), 7.55-7.48 (m, 1H), 7.26-7.19 (m, 2H), 5.40-5.19 (m, 3H), 4.36-4.22 (m, 2H), 4.12-3.89 (m, 3H), 3.52 (d, J=1.2 Hz, 3H), 3.41-3.11 (m, 3H), 3.08-2.94 (m, 1H), 2.80 (t, J=5.2 Hz, 3H), 2.65-2.48 (m, 1H), 2.43-2.08 (m, 8H), 2.03-1.70 (m, 9H), 1.68-1.57 (m, 1H), 0.85 (q, J=7.2 Hz, 3H); LCMS (ESI, M+1): m/z=707.4.

Step D. N-acetyl-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazocane-4-carboxamide: To a mixture of 1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazocane-4-carboxamide (250 mg, 1.0 eq.) in THF (3 mL) was added NaH (42.4 mg, 60% purity, 3.0 eq.) at 0 °C. The reaction was stirred at 25 °C for 0.5 hour. To the above mixture was added a solution of acetyl chloride (55.5 mg, 2.0 eq.) in THF (2 mL) at 0 °C. The reaction was stirred at 40 °C for 11.5 hours. The mixture was quenched with water (5 mL) and extracted with ethyl acetate (3×5 mL). The combined organic layers were dried over anhydrous sodium sulfate, concentrated and purified with reversed phase flash chromatography [0.1% formic acid condition] to afford the title compound (50.0 mg, 17% yield) as yellow solid; LCMS (ESI, M+1): m/z=749.5.

Step E. N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazocane-4-carboxamide: To a solution of N-acetyl-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazocane-4-carboxamide (50.0 mg, 1.0 eq.) in DCM (0.5 mL) was added TFA (767 mg, 101 eq.). The reaction was stirred at 15 °C for 0.5 hour. The mixture was concentrated, basified with saturated aqueous NaHCO₃ (2 mL) and extracted with ethyl acetate (2×2 mL). The combined organic layers were dried over anhydrous sodium sulfate, concentrated and purified with prep-HPLC [Phenomenex luna C18 150×25 mm×10 μm; A: water (FA), B: ACN, B %: 18%-48% over 9 min]. The desired fractions were basified with NaHCO₃ and extracted with ethyl acetate (2×8 mL). The combined organic layers were dried over anhydrous sodium sulfate, concentrated and purified with reversed phase flash chromatography [water (0.1% TFA)/acetonitrile]. The desired fractions were basified with NaHCO₃ and extracted with ethyl acetate (2×7 mL). The combined organic layers were dried over anhydrous sodium sulfate and concentrated to afford the title compound (5.86 mg, 12% yield) as white solid; ¹H NMR (400 MHz, CHLOROFORM-d₃) δ=9.10-8.99 (m, 1H), 7.54 (dd, J=6.0, 9.6 Hz, 1H), 7.21-7.05 (m, 3H), 5.46-5.19 (m, 1H), 4.38-4.17 (m, 2H), 4.11-3.95 (m, 2H), 3.93-3.77 (m, 1H), 3.69-3.58 (m, 1H), 3.51-3.21 (m, 2H), 3.20-3.14 (m, 3H), 3.11-2.96 (m, 1H), 2.57-2.46 (m, 1H), 2.35-2.31 (m, 3H), 2.28-2.08 (m, 6H), 2.05-1.95 (m, 3H), 1.94-1.46 (m, 8H), 0.87-0.78 (m, 3H); LCMS (ESI, M+1): m/z=705.3.

### Example 276

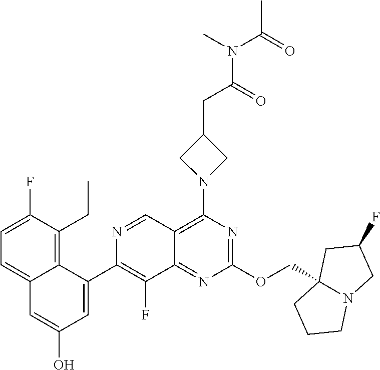 [SMILES: CCc1c(F)ccc2cc(O)cc(-c3ncc4c(N5CC(CC(=O)N(C)C(C)=O)C5)nc(OC[C@@]56CCCN5C[C@H](F)C6)nc4c3F)c12]

### N-acetyl-2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)-N-methylacetamide

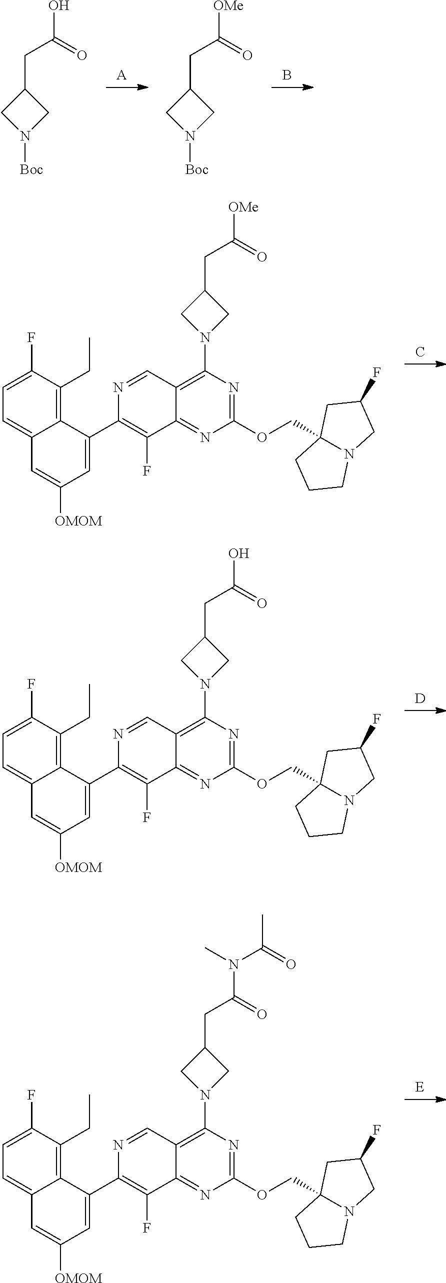

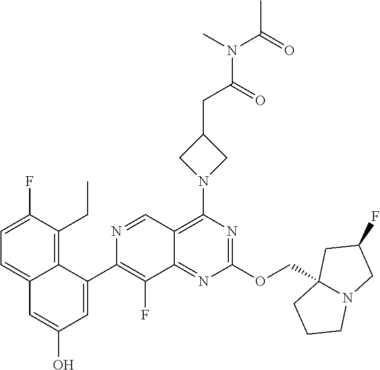 [SMILES: CCc1c(F)ccc2cc(O)cc(-c3ncc4c(N5CC(CC(=O)N(C)C(C)=O)C5)nc(OC[C@@]56CCCN5C[C@H](F)C6)nc4c3F)c12]

Step A. methyl 2-(azetidin-3-yl)acetate: A solution of 2-(1-(tert-butoxycarbonyl) azetidin-3-yl) acetic acid (500 mg, 1.0 eq.) in HCl·MeOH (2M, 5 mL) was stirred at 25 °C for 12 hours. The mixture was concentrated to afford the title compound (412 mg, crude) as colorless oil.

Step B. methyl 2-(1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)acetate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (150 mg, 1.0 eq.) in DMF (4 mL) were added DIEA (280 mg, 8.0 eq.) and PyBOP (211 mg, 1.5 eq.). The reaction was stirred at 25 °C for 0.5 hour. Methyl 2-(azetidin-3-yl)acetate (65.5 mg, 1.5 equiv, HCl) was added. The reaction was stirred at 25 °C for 1 hour. The mixture was filtered, and the filtrate was purified with prep-HPLC [column: Waters Xbridge 150×25 mm×5 μm; mobile phase: water (NH₄HCO₃)-ACN; gradient: 52%-82% B over 9 min] to afford the title compound (84.0 mg, 45% yield) as colorless oil; LCMS (ESI, M+1): m/z=666.4.

Step C. 2-(1-(7-(8-ethyl-7-fluoro-3-(methoxymethoxy)naphthalen-1-yl)-8-fluoro-2-yl) azetidin-3-yl) acetic acid: To a solution of methyl 2-(1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)acetate (84.0 mg, 1.0 eq.) in MeOH (2 mL) was added LiOH·H₂O (2M, 2 mL). The reaction was stirred at 25 °C for 1 hour. The mixture was diluted with water (5 mL), acidified with solid citric acid (pH ~5) and extracted with ethyl acetate (2×10 mL). The combined organic layers were washed with brine (10 mL), dried over anhydrous sodium sulfate and concentrated to afford the title compound (89.0 mg, crude) as colorless oil; LCMS (ESI, M+1): m/z=652.2.

Step D. N-acetyl-2-(1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)-N-methylacetamide: To a solution of 2-(1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl) acetic acid (79.0 mg, 1.0 eq.) and N-methylacetamide (44.3 mg, 5.0 eq.) in 2-MeTHF (4 mL) were added CMPI (155 mg, 5.0 eq.) and DIEA (78.3 mg, 5.0 eq.). The reaction was stirred at 80 °C for 1 hour. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge 150×25 mm×5 μm; mobile phase: water (NH₄HCO₃)-ACN; gradient: 48%-78% B over 9 min] to afford the title compound (62.0 mg, 70% yield) as white solid; LCMS (ESI, M+1): m/z=707.3.

Step E. N-acetyl-2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)-N-methylacetamide: To a solution of N-acetyl-2-(1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) azetidin-3-yl)-N-methylacetamide (62.0 mg, 1.0 eq.) in DCM (3 mL) was added TFA (1 mL). The reaction was stirred at 25 °C for 1 hour. The mixture was concentrated, diluted with ethyl acetate (5 mL) and neutralized with DIEA at −40 °C. The mixture was washed with water (5 mL), dried over anhydrous sodium sulfate, concentrated and purified with prep-HPLC [column: Phenomenex luna C18 150×25 mm×10 μm; mobile phase: water (FA)-ACN; gradient: 18%-48% B over 9 min] to afford the title compound (4.40 mg, 7.4% yield, 0.12HCOOH) as white solid; ¹H NMR (400 MHz, DMSO-d₆) δ=9.93 (s, 1H), 8.93 (s, 1H), 7.76 (dd, J=6.0, 9.2 Hz, 1H), 7.40-7.30 (m, 2H), 6.99 (s, 1H), 5.37-5.20 (m, 1H), 5.00 (s, 1H), 4.61-4.40 (m, 2H), 4.15 (d, J=10.0 Hz, 1H), 4.05 (d, J=9.6 Hz, 2H), 3.26 (d, J=6.8 Hz, 2H), 3.15 (s, 6H), 3.05 (s, 1H), 2.90-2.80 (m, 1H), 2.40-2.28 (m, 4H), 2.17-1.99 (m, 4H), 1.91-1.74 (m, 3H), 0.72 (t, J=7.2 Hz, 3H); LCMS (ESI, M+1): m/z=663.3.

### Example 277

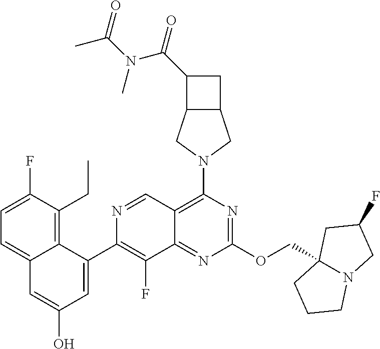 [SMILES: CCc1c(F)ccc2cc(O)cc(-c3ncc4c(N5CC6CC(C(=O)N(C)C(C)=O)C6C5)nc(OC[C@@]56CCCN5C[C@H](F)C6)nc4c3F)c12]

### N-acetyl-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.0]heptane-6-carboxamide

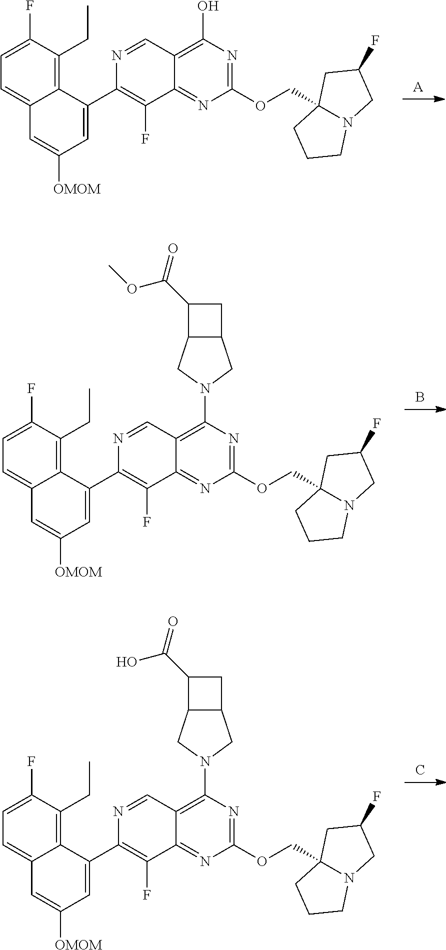

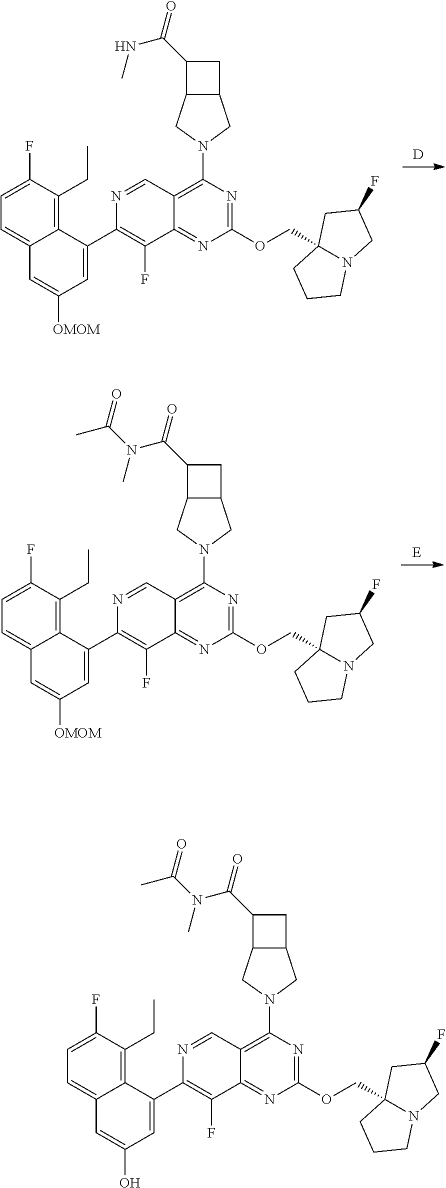

Step A. methyl 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate: To a mixture of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 eq.), DIEA (291 mg, 2.5 eq.) and DMAP (2.20 mg, 0.02 eq.) in DCM (5.0 mL) was added 2-nitrobenzenesulfonyl chloride (220 mg, 1.1 eq.) at 0 °C The reaction was stirred at 0 °C for 2 hours. Then methyl 3-azabicyclo[3.2.0]heptane-6-carboxylate (207 mg, 1.2 equiv, HCl) was added. The reaction was stirred at 0 °C for 2 hours. The mixture was diluted with water (4.00 mL) and extracted with DCM (3×3.00 mL). The combined organic layers were dried over anhydrous sodium sulfate, concentrated, and purified with reversed phase flash chromatography [C18, 0.1% formic acid condition] to afford the title compound (440 mg, 68% yield) as yellow solid; LCMS (ESI, M+1): m/z=692.6.

Step B. 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid: To a mixture of methyl 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylate (426 mg, 1.0 eq.) in THF (1.5 mL), H₂O (1.5 mL) and MeOH (1.0 mL) was added LiOH·H₂O (77.5 mg, 3.0 eq.). The reaction was stirred at 20 °C for 2 hours. The mixture was filtered and washed with MeOH (1.0 mL) and purified with reversed phase flash chromatography [C18, 0.1% formic acid condition] to afford the title compound (400 mg, 96% yield) as yellow solid; LCMS (ESI, M+1): m/z=678.3.

Step C. 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.0]heptane-6-carboxamide: To a mixture of 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.0]heptane-6-carboxylic acid (350 mg, 1.0 eq.), HATU (393 mg, 2.0 eq.) and DIEA (200 mg, 3.0 eq.) in DMF (4.5 mL) was added methanamine hydrochloride (69.7 mg, 2.0 eq.). The reaction was stirred at 20 °C for 12 hours. The residue was filtered and washed with DMF (1.0 mL) and purified with reversed phase flash chromatography [C18, 0.1% formic acid condition] to afford the title compound (356 mg, 99% yield) as yellow solid; LCMS (ESI, M+1): m/z=691.4.

Step D. N-acetyl-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.0]heptane-6-carboxamide: To a mixture of 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.0]heptane-6-carboxamide (300 mg, 1.0 eq.) in THF (3.0 mL) was added NaH (69.5 mg, 60% purity, 4.0 eq.). The reaction was stirred at 0 °C for 0.5 hour. Then acetyl chloride (136 mg, 4.0 eq.) was added to above mixture at 0 °C. The reaction was stirred at 40 °C for 36 hours. The mixture was diluted with water (10.0 mL) and extracted with ethyl acetate (3×3.00 mL). The combined organic layers were dried over anhydrous sodium sulfate, concentrated and purified with reversed phase flash chromatography [C18, 0.1% formic acid condition] to afford the title compound (40.0 mg, 12% yield) as yellow solid; LCMS (ESI, M+1): m/z=733.4.

Step E. N-acetyl-3-(7-(8-ethyl-7-fluoro-3-hydroxynaphthalen-1-yl)-8-fluoro-2-yl)-N-methyl-3-azabicyclo[3.2.0]heptane-6-carboxamide: To a solution of N-acetyl-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.0]heptane-6-carboxamide (40.0 mg, 1.0 eq.) in DCM (0.2 mL) was added TFA (768 mg, 120 eq.). The reaction was stirred at 20 °C for 0.5 hour. The mixture was basified with saturated NaHCO₃ aqueous solution (10.0 mL) and extracted with ethyl acetate (3×3.00 mL). The combined organic layers were dried over anhydrous sodium sulfate, concentrated and purified with prep-HPLC [Waters xbridge 150×25 mm 10 μm; A: water (NH₄HCO₃); B: ACN; gradient: 48%-68% B over 8 min] to afford the title compound (6.98 mg, 18% yield) as white solid; ¹H NMR (400 MHz, METHANOL-d₄) δ=9.19 (d, J=2.0 Hz, 1H), 7.68 (dd, J=5.6, 9.2 Hz, 1H), 7.30 (d, J=2.8 Hz, 1H), 7.25 (t, J=9.6 Hz, 1H), 7.06 (dd, J=2.4, 5.6 Hz, 1H), 5.39-5.21 (m, 1H), 4.49-4.15 (m, 4H), 4.14-3.80 (m, 3H), 3.69-3.57 (m, 1H), 3.27-3.12 (m, 7H), 3.05-2.96 (m, 1H), 2.56-2.43 (m, 1H), 2.41-2.31 (m, 5H), 2.30-2.09 (m, 4H), 2.04-1.94 (m, 2H), 1.93-1.83 (m, 1H), 0.86-0.74 (m, 3H); LCMS (ESI, M+1): m/z=689.4.

### Example 278

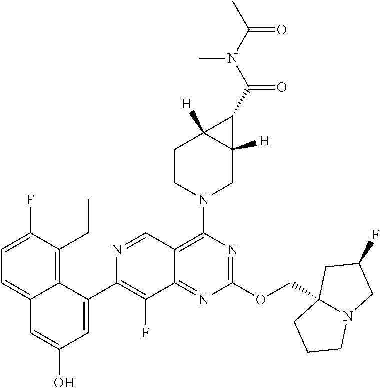 [SMILES: CCc1c(F)ccc2cc(O)cc(-c3ncc4c(N5CC[C@H]6[C@@H](C5)[C@H]6C(=O)N(C)C(C)=O)nc(OC[C@@]56CCCN5C[C@H](F)C6)nc4c3F)c12]

### (1R,6S)-N-acetyl-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[4.1.0]heptane-7-carboxamide

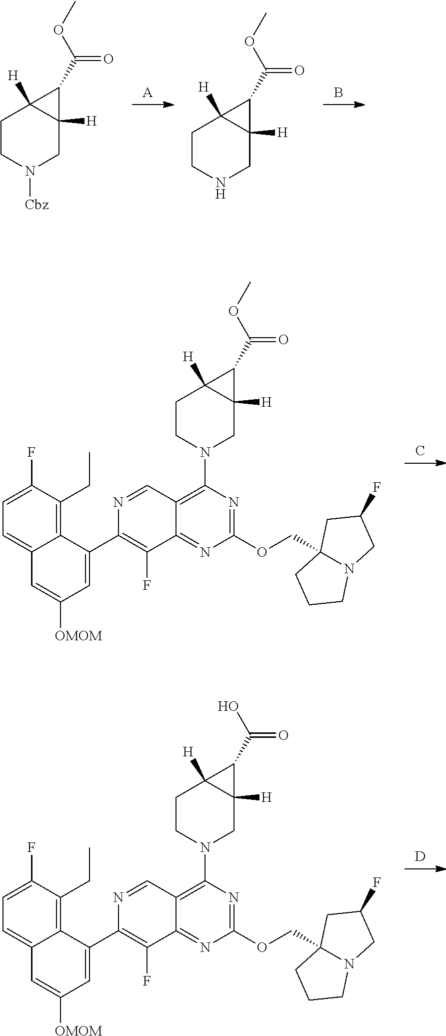

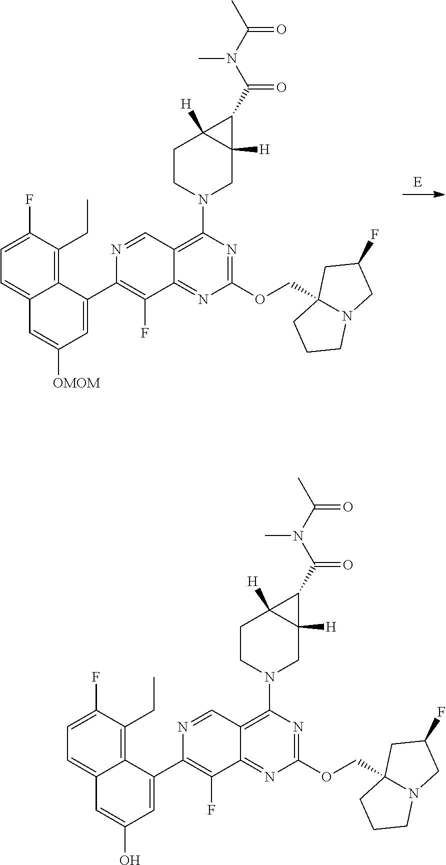

Step A. methyl (1R,6S,7S)-3-azabicyclo[4.1.0]heptane-7-carboxylate: To a solution of 3-benzyl 7-methyl (1R,6S,7S)-3-azabicyclo[4.1.0]heptane-3,7-dicarboxylate (500 mg, 1.0 eq.) in MeOH (10 mL) was added Pd/C (1.00 g, 10% purity) under nitrogen atmosphere. The suspension was degassed and purged with hydrogen for three times. The reaction was stirred under hydrogen (15 PSI) at 25 °C for 1 hour. The mixture was filtered and concentrated to afford the title compound (204 mg, crude) as colorless oil; ¹H NMR (400 MHz, CHLOROFORM-d) δ=3.65 (s, 3H), 3.21-3.13 (m, 1H), 3.10-3.02 (m, 1H), 2.63-2.55 (m, 1H), 2.55-2.46 (m, 1H), 1.96-1.88 (m, 2H), 1.75-1.63 (m, 3H), 1.63-1.56 (m, 1H).

Step B. methyl (1R,6S,7S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (200 mg, 1.0 eq.) and methyl (1R,6S,7S)-3-azabicyclo[4.1.0]heptane-7-carboxylate (112 mg, 2.0 eq.) in DMF (2 mL) were added DIEA (373 mg, 8.0 eq.) and PyBOP (282 mg, 1.5 eq.). The reaction was stirred at 25 °C for 2 hours. The mixture was filtered, and the filtrate was purified with prep-HPLC [column: Waters Xbridge Prep OBD C18 150×40 mm×10 μm; mobile phase: water (NH₄HCO₃)-ACN; gradient: 52%-82% B over 15 min] to afford the title compound (140 mg, 55% yield) as colorless oil; LCMS (ESI, M+1): m/z=692.4.

Step C. (1R,6S,7S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid: To a methyl (1R,6S,7S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylate (84.0 mg, 1.0 eq.) in MeOH (3 mL) was added LiOH·H₂O (2M, 1.2 mL). The reaction was stirred at 25 °C for 4 hours. The mixture was diluted with water (5 mL), acidified with solid citric acid (pH 5) and extracted with ethyl acetate (2×10 mL). The combined organic layers were washed with brine (10 mL), dried over anhydrous sodium sulfate, concentrated and purified with prep-HPLC [column: Phenomenex luna C18 150×25 mm×10 μm; mobile phase: water (FA)-ACN; gradient: 25%-45% B over 10 min] to afford the title compound (40.0 mg, 29% yield) as white solid; LCMS (ESI, M+1): m/z=678.4.

Step D. (1R,6S,7S)-N-acetyl-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[4.1.0]heptane-7-carboxamide: To a solution of (1R,6S,7S)-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[4.1.0]heptane-7-carboxylic acid (40.0 mg, 1.0 eq.) and N-methylacetamide (21.6 mg, 5.0 eq.) in 2-MeTHF (1 mL) were added CMPI (75.4 mg, 5.0 eq.) and DIEA (38.1 mg, 5.0 eq.). The reaction was stirred at 80 °C for 1 hour. The mixture was diluted with water (5 mL) and extracted with ethyl acetate (2×10 mL). The combined organic layers were washed with brine (10 mL), dried over anhydrous sodium sulfate, concentrated and purified with prep-HPLC [column: Phenomenex luna C18 150×25 mm×10 μm; mobile phase: water (FA)-ACN; gradient: 25%-55% B over 9 min] to afford the title compound (15.0 mg, 33% yield) as white solid; LCMS (ESI, M+1): m/z=733.4.

Step E. (1R,6S,7S)-N-acetyl-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[4.1.0]heptane-7-carboxamide: To a solution of (1R,6S,7S)-N-acetyl-3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[4.1.0]heptane-7-carboxamide (15.0 mg, 1.0 eq.) in DCM (0.3 mL) was added TFA (0.1 mL). The reaction was stirred at 25 °C for 1 hour. The mixture was concentrated, diluted with ethyl acetate (5 mL) and neutralized with DIEA at −40 °C. The mixture was washed with water (5 mL), dried over anhydrous sodium sulfate, concentrated and purified with prep-HPLC [column: Phenomenex luna C18 150×25 mm×10 μm; mobile phase: water (FA)-ACN; gradient: 23%-43% B over 10 min] to afford the title compound (8.40 mg, 58% yield, 0.4HCOOH) as white solid; ¹H NMR (400 MHz, DMSO-d₆) δ=9.06 (d, J=5.2 Hz, 1H), 7.76 (dd, J=6.0, 9.2 Hz, 1H), 7.39-7.30 (m, 2H), 7.02 (dd, J=2.4, 6.8 Hz, 1H), 5.38-5.18 (m, 1H), 4.41-4.27 (m, 1H), 4.25-4.16 (m, 1H), 4.15-4.02 (m, 2H), 3.95-3.85 (m, 1H), 3.71-3.53 (m, 2H), 3.18 (d, J=7.2 Hz, 3H), 3.12-3.05 (m, 2H), 3.01 (br s, 1H), 2.86-2.79 (m, 1H), 2.37-2.27 (m, 5H), 2.15-1.98 (m, 5H), 1.92-1.74 (m, 5H), 0.7-0.69 (m, 3H); LCMS (ESI, M+1): m/z=689.2.

### Example 279

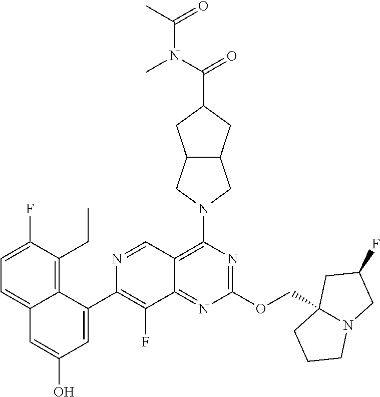 [SMILES: CCc1c(F)ccc2cc(O)cc(-c3ncc4c(N5CC6CC(C(=O)N(C)C(C)=O)CC6C5)nc(OC[C@@]56CCCN5C[C@H](F)C6)nc4c3F)c12]

### N-acetyl-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyloctahydrocyclopenta[c]pyrrole-5-carboxamide

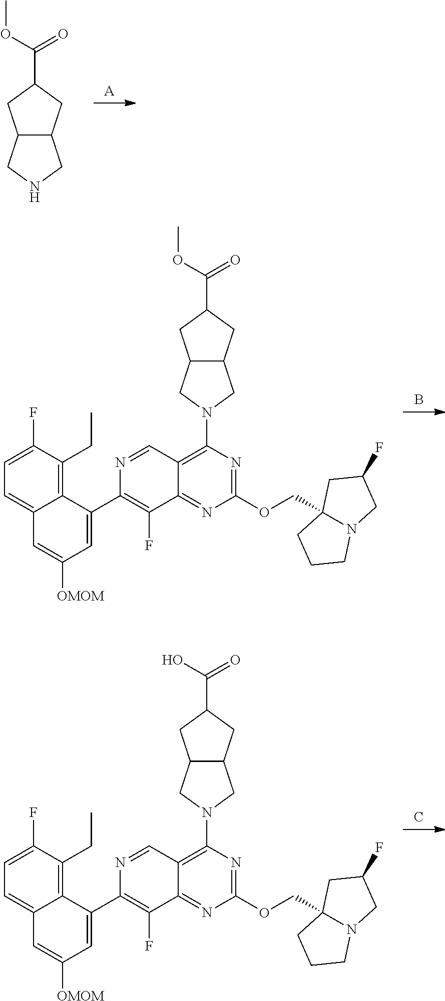

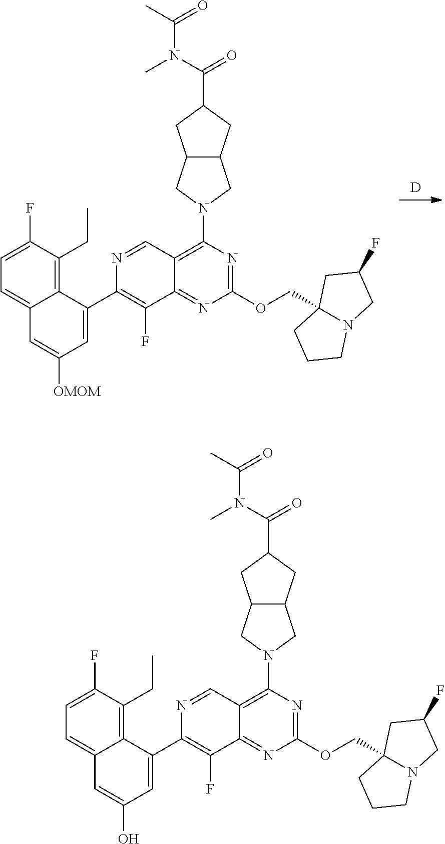

Step A. methyl 2-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (500 mg, 1.0 eq.) and methyl octahydrocyclopenta[c]pyrrole-5-carboxylate (278 mg, 1.5 equiv, HCl salt) in DMF (10 mL) were added PyBOP (604 mg, 1.5 eq.) and DIEA (1.17 g, 10 eq.). The reaction was stirred at 25 °C for 1 hour. The mixture was filtered, and the filtrate was purified with reversed phase flash chromatography [C18, 0.1% formic acid condition] to afford the title compound (510 mg, 77% yield) as white solid; LCMS (ESI, M+1): m/z=706.3.

Step B. 2-(7-(8-ethyl-7-fluoro-3-(methoxymethoxy)naphthalen-1-yl)-8-fluoro-2-yl)octahydrocyclopenta[c]pyrrole-5-carboxylic acid: To a solution of methyl 2-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylate (500 mg, 1.0 eq.) in MeOH (25 mL) was added LiOH·H₂O (2 M in H₂O, 19 mL, 53.6 eq.). The reaction was stirred at 25 °C for 0.5 hour. The mixture was filtered, and the filtrate was purified with reversed phase flash chromatography [C18, 0.1% formic acid condition] to afford the title compound (445 mg, 88% yield) as yellow solid; LCMS (ESI, M+1): m/z=692.4.

Step C. N-acetyl-2-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyloctahydrocyclopenta[c]pyrrole-5-carboxamide: To a solution of 2-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)octahydrocyclopenta[c]pyrrole-5-carboxylic acid (100 mg, 1.0 eq.) and N-methylacetamide (21.1 mg, 2.0 eq.) in 2-methyltetrahydrofuran (1.0 mL) were added DIEA (187 mg, 10 eq.) and CMPI (148 mg, 4.0 eq.). The reaction was stirred at 80 °C for 1 hour. The mixture was diluted with water (3 mL) and extracted with ethyl acetate (3×2 mL). The organic layers were dried over anhydrous sodium sulfate and concentrated to afford the title compound (100 mg, crude) as yellow oil.

Step D. N-acetyl-2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyloctahydrocyclopenta[c]pyrrole-5-carboxamide: To a solution of N-acetyl-2-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyloctahydrocyclopenta[c]pyrrole-5-carboxamide (100 mg, 1.0 eq.) in DCM (1 mL) was added TFA (2.30 g, 151 eq.). The reaction was stirred at 25 °C for 0.5 hour. The mixture was concentrated, basified with saturated NaHCO₃ solution (6 mL) and extracted with ethyl acetate (3×5 mL). The combined organic layers were dried over anhydrous sodium sulfate, concentrated and purified with reversed phase flash chromatography [C18, H₂O condition] and prep-HPLC [Welch Xtimate C18 150×25 mm×5 μm; A: water (FA), B: ACN, B %: 20%-40% over 10 min] to afford the title compound (5.46 mg, 5.7% yield, 0.09 HCOOH) as pink solid; ¹H NMR (400 MHz, CHLOROFORM-d₃) δ=9.12 (t, J=8.8 Hz, 1H), 7.52 (dd, J=6.4, 8.8 Hz, 1H), 7.22-7.11 (m, 2H), 7.04-6.90 (m, 1H), 5.46-5.20 (m, 1H), 4.51-4.26 (m, 2H), 4.25-4.02 (m, 2H), 3.96-3.83 (m, 1H), 3.82-3.65 (m, 2H), 3.59-3.38 (m, 2H), 3.33-3.23 (m, 1H), 3.23-3.16 (m, 3H), 3.08-3.01 (m, 1H), 2.97-2.90 (m, 1H), 2.84-2.74 (m, 1H), 2.57-2.42 (m, 2H), 2.40 (s, 3H), 2.36-2.26 (m, 2H), 2.26-2.18 (m, 2H), 2.16-2.09 (m, 1H), 2.01 (br d, J=3.6 Hz, 3H), 1.96-1.87 (m, 2H), 0.78-0.83 (m, 3H); LCMS (ESI, M+1): m/z=703.2.

### Example 280

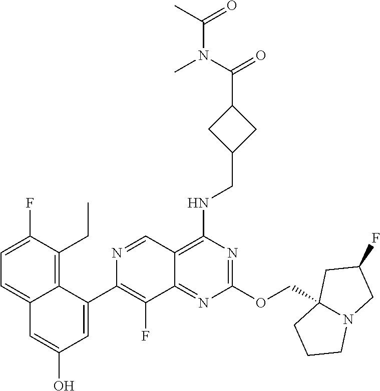 [SMILES: CCc1c(F)ccc2cc(O)cc(-c3ncc4c(NCC5CC(C(=O)N(C)C(C)=O)C5)nc(OC[C@@]56CCCN5C[C@H](F)C6)nc4c3F)c12]

### N-acetyl-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclobutane-1-carboxamide

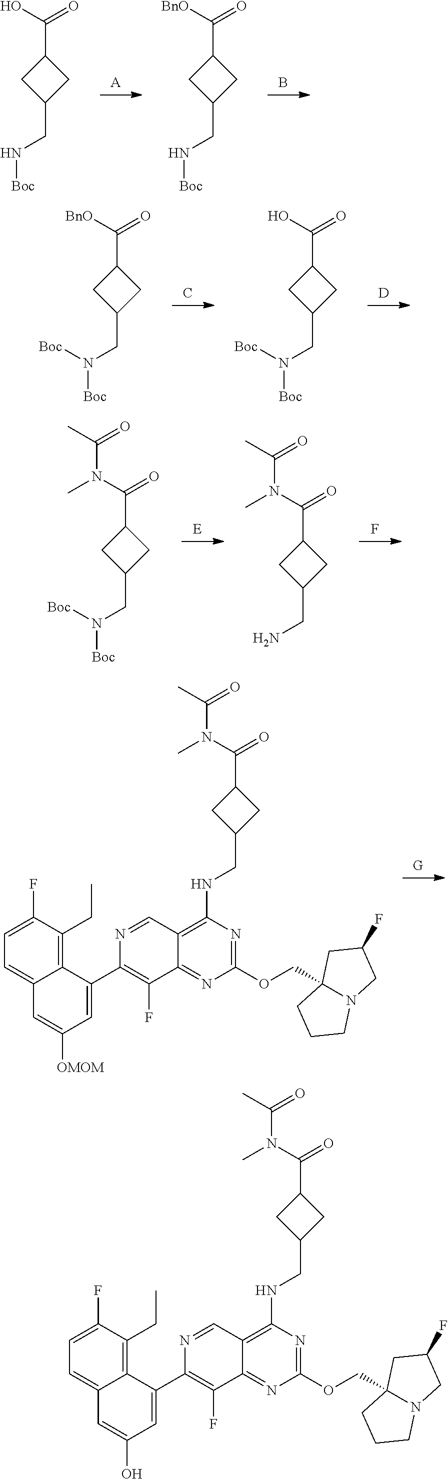

Step A. benzyl 3-(((tert-butoxycarbonyl)amino)methyl)cyclobutane-1-carboxylate: To a solution of 3-(((tert-butoxycarbonyl)amino)methyl)cyclobutane-1-carboxylic acid (1.8 g, 1.0 eq.) in DCM (18 mL) and DMF (1.0 mL) were added HATU (4.48 g, 1.5 eq.) and DIEA (3.04 g, 3.0 eq.) at 0 °C The mixture was stirred at 25 °C for 10 minutes. To the above mixture was added phenylmethanol (1.27 g, 1.5 eq.) at 0 °C. The reaction was stirred at 25 °C for 50 minutes. The mixture was diluted with water (50 mL) and extracted with ethyl acetate (2×20 mL). The organic layers were dried over anhydrous sodium sulfate, concentrated, and purified with reversed phase flash chromatography [C18, 0.1% formic acid condition] to afford the title compound (2.3 g, 84% yield) as yellow oil; LCMS (ESI, M+23): m/z=342.1.

Step B. benzyl 3-[[bis(tert-butoxycarbonyl)amino]methyl]cyclobutanecarboxylate: To a solution of benzyl 3-(((tert-butoxycarbonyl)amino)methyl)cyclobutane-1-carboxylate (2.3 g, 1.0 eq.) in Di-tert-butyl dicarbonate (23 mL) was added DMAP (44.0 mg, 0.05 eq.). The reaction was stirred at 60 °C for 0.5 hour. The mixture was diluted with water (50 mL) and extracted with ethyl acetate (2×20 mL). The mixture was filtered, and the filtrate was purified with reversed phase flash chromatography [C18, 0.1% formic acid condition] to afford the title compound (2.7 g, 81% yield) as yellow oil; LCMS (ESI, M+23): m/z=442.2.

Step C. 3-[[bis(tert-butoxycarbonyl)amino]methyl]cyclobutanecarboxylic acid: To a solution of benzyl 3-[[bis(tert-butoxycarbonyl)amino]methyl]cyclobutanecarboxylate (2.4 g, 1.0 eq.) in MeOH (25 mL) was added Pd/C (1 g, 10%) under nitrogen. The reaction was stirred at 25 °C for 1 hour under H₂ (15 psi). The mixture was filtered and concentrated to afford the title compound (1.6 g, crude) as yellow oil.

Step D. tert-butyl N-[[3-[acetyl(methyl)carbamoyl]cyclobutyl]methyl]-N-tert-butoxycarbonyl-carbamate: To a solution of 3-[[bis(tert-butoxycarbonyl)amino]methyl]cyclobutanecarboxylic acid (300 mg, 1.0 eq.) and N-methylacetamide (133 mg, 2.0 eq.) in 2Me-THF (3.0 mL) were added DIEA (1.18 g, 10 eq.) and CMPI (930 mg, 4.0 eq.). The reaction was stirred at 80 °C for 1 hour. The mixture was diluted with water (3 mL) and extracted with ethyl acetate (3×2 mL). The organic layers were dried over anhydrous sodium sulfate, concentrated, and purified with reversed phase flash chromatography [C18, 0.1% formic acid condition] to afford the title compound (120 mg, 77% yield) as colorless oil; LCMS (ESI, M+23): m/z=407.1.

Step E. N-acetyl-3-(aminomethyl)-N-methylcyclobutane-1-carboxamide: To a solution of 3-[[bis(tert-butoxycarbonyl)amino]methyl]cyclobutanecarboxylic acid (100 mg, 1.0 eq.) in DCM (1 mL) was added TFA (1.54 g, 52 eq.). The reaction was stirred at 25 °C for 10 minutes. The mixture was concentrated to afford the title compound (70 mg, crude, TFA salt) as yellow oil.

Step F. N-acetyl-3-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclobutane-1-carboxamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (100 mg, 1.0 eq.) and N-acetyl-3-(aminomethyl)-N-methylcyclobutane-1-carboxamide (70 mg, 1.3 equiv, TFA salt) in DMF (1 mL) were added PyBOP (141 mg, 1.5 eq.) and DIEA (233 mg, 10 eq.). The reaction was stirred at 25 °C for 12 hours. The mixture was diluted with water (3 mL) and extracted with ethyl acetate (3×2 mL). The organic layers were dried over anhydrous sodium sulfate and concentrated to afford the title compound (100 mg, crude) as yellow oil.

Step G. N-acetyl-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclobutane-1-carboxamide: To a solution of N-acetyl-3-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclobutane-1-carboxamide (100 mg, 1.0 eq.) in DCM (1 mL) was added TFA (1.54 g, 97 eq.). The reaction was stirred at 25 °C for 10 minutes. The mixture was concentrated. The residue was basified with saturated NaHCO₃ solution (6 mL) and extracted with ethyl acetate (3×5 mL). The combined organic layers were dried over anhydrous sodium sulfate, concentrated and purified with reversed phase flash chromatography [C18, H₂O condition] to afford the title compound (16.8 mg, 17% yield) as white solid; ¹H NMR (400 MHz, CHLOROFORM-d₃) δ=9.19-9.02 (m, 1H), 7.53 (br s, 1H), 7.45 (br dd, J=6.4, 7.2 Hz, 1H), 7.17-7.08 (m, 1H), 7.07-6.98 (m, 1H), 6.98-6.73 (m, 1H), 5.40-5.13 (m, 1H), 4.40-4.17 (m, 2H), 3.72-3.44 (m, 3H), 3.39-3.17 (m, 3H), 3.16-3.09 (m, 3H), 3.05-2.93 (m, 1H), 2.66 (br dd, J=4.0, 7.6 Hz, 1H), 2.49-2.40 (m, 3H), 2.38 (d, J=1.2 Hz, 3H), 2.28-2.05 (m, 6H), 1.94 (br d, J=12.4 Hz, 3H), 0.86-0.64 (m, 3H); LCMS (ESI, M+1): m/z=677.4.

### Example 281

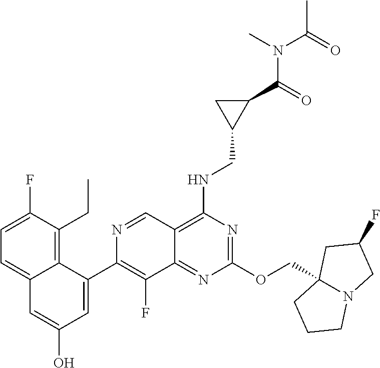 [SMILES: CCc1c(F)ccc2cc(O)cc(-c3ncc4c(NC[C@@H]5C[C@H]5C(=O)N(C)C(C)=O)nc(OC[C@@]56CCCN5C[C@H](F)C6)nc4c3F)c12]

### trans-N-acetyl-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclopropane-1-carboxamide

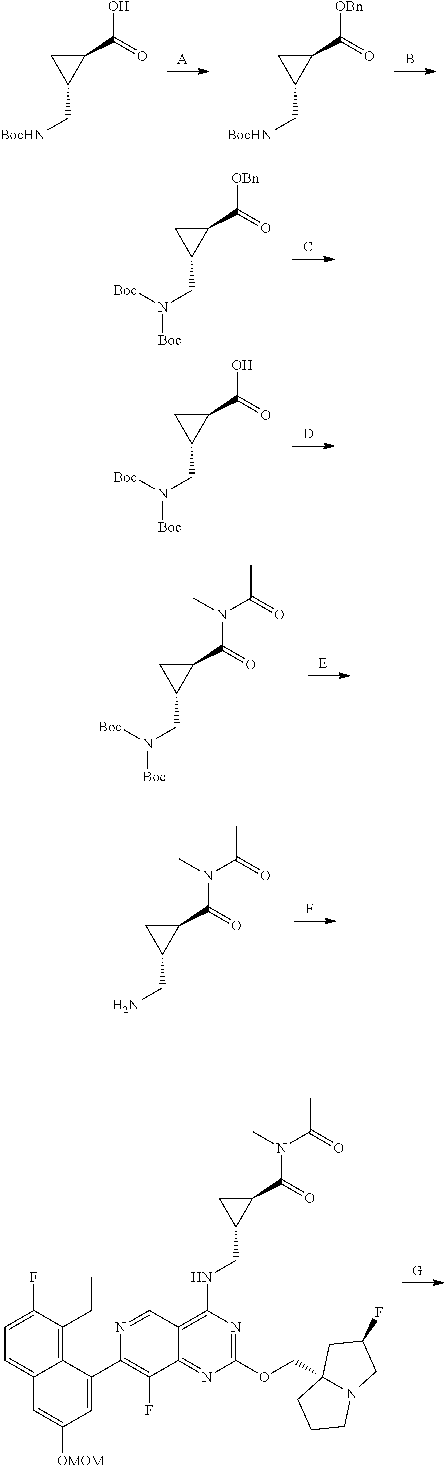

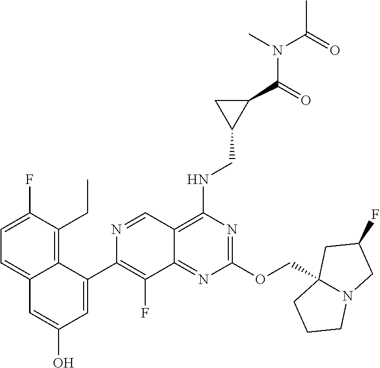 [SMILES: CCc1c(F)ccc2cc(O)cc(-c3ncc4c(NC[C@@H]5C[C@H]5C(=O)N(C)C(C)=O)nc(OC[C@@]56CCCN5C[C@H](F)C6)nc4c3F)c12]

Step A. benzyl trans-2-(((tert-butoxycarbonyl)amino)methyl)cyclopropane-1-carboxylate: To a solution of trans-2-(((tert-butoxycarbonyl)amino)methyl)cyclopropane-1-carboxylic acid (2.00 g, 1.0 eq.), BnOH (2.01 g, 2.0 eq.) and DMAP (113 mg, 0.1 eq.) in DCM (40 mL) was added DCC (2.30 g, 1.2 eq.). The reaction was stirred at 25 °C for 2 hours. The mixture was diluted with water (50 mL) and extracted with DCM (2×50 mL). The combined organic layers were washed with brine (50 mL), dried over anhydrous sodium sulfate, concentrated and purified with column chromatography [SiO₂, petroleum ether/ethyl acetate=I/O to 20/1] to afford the title compound (3.20 g, crude) as colorless oil; LCMS (ESI, M+23): m/z=328.1.

Step B. benzyl trans-2-((bis(tert-butoxycarbonyl)amino)methyl)cyclopropane-1-carboxylate: To a solution of benzyl trans-2-(((tert-butoxycarbonyl)amino)methyl)cyclopropane-1-carboxylate (3.20 g, 1.0 eq.) in ACN (50 mL) were added Boc₂O (3.20 g, 2.0 eq.), DMAP (89.6 mg, 0.1 eq.) and TEA (2.23 g, 3.0 eq.). The reaction was stirred at 80 °C for 1 hour. The mixture was concentrated, then diluted with water (100 mL) and extracted with ethyl acetate (2×100 mL). The combined organic layers were washed with brine (100 mL), dried over anhydrous sodium sulfate, concentrated, and purified with column chromatography [SiO₂, petroleum ether/ethyl acetate=100/1 to 10/1] to afford the title compound (2.70 g, 64% yield) as yellow oil; LCMS (ESI, M+23): m/z=428.2.

Step C. trans-2-((bis(tert-butoxycarbonyl)amino)methyl)cyclopropane-1-carboxylic acid: To a solution of benzyl trans-2-((bis(tert-butoxycarbonyl)amino)methyl)cyclopropane-1-carboxylate (2.70 g, 1.0 eq.) in MeOH (30 mL) was added Pd/C (10%, 1.00 g) under nitrogen atmosphere. The suspension was degassed and purged with hydrogen for three times. The reaction was stirred under hydrogen (15 Psi) at 25 °C for 1 hour. The mixture was filtered and the filtrate was concentrated to afford the title compound (2.10 g, crude) as white solid; ¹H NMR (400 MHz, METHANOL-d₄) δ=3.66 (dd, J=6.0, 14.8 Hz, 1H), 3.47 (dd, J=7.6, 14.8 Hz, 1H), 1.74-1.67 (m, 1H), 1.66-1.62 (m, 1H), 1.53 (s, 18H), 1.13-1.08 (m, 1H), 0.96-0.91 (m, 1H).

Step D. tert-butyl N-((trans-2-(acetyl(methyl)carbamoyl)cyclopropyl)methyl)-N-tert-butoxycarbonyl-carbamate: To a solution of trans-2-((bis(tert-butoxycarbonyl)amino)methyl)cyclopropane-1-carboxylic acid (1.00 g, 1.0 eq.) in 2-MeTHF (20 mL) were added N-methylacetamide (463 mg, 2.0 eq.), CMPI (4.05 g, 5.0 eq.) and DIEA (2.05 g, 5.0 eq.) The reaction was stirred at 80 °C for 1 hour. The mixture was diluted with water (30 mL) and extracted with ethyl acetate (2×30 mL). The combined organic layers were washed with brine (30 mL), dried over anhydrous sodium sulfate, concentrated, and purified with column chromatography [SiO₂, petroleum ether/ethyl acetate=50/1 to 10/1] to afford the title compound (160 mg, 14% yield) as colorless oil; LCMS (ESI, M+23): m/z=393.2.

Step E. trans-N-acetyl-2-(aminomethyl)-N-methylcyclopropane-1-carboxamide: To a solution of tert-butyl N-((trans-2-(acetyl(methyl)carbamoyl)cyclopropyl)methyl)-N-tert-butoxycarbonyl-carbamate (160 mg, 1.0 eq.) in DCM (3 mL) was added TFA (1 mL). The reaction was stirred at 25 °C for 1 hour. The mixture was concentrated to afford the title compound (70 mg, crude) as colorless oil.

Step F. trans-N-acetyl-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclopropane-1-carboxamide: To a solution of trans-N-acetyl-2-(aminomethyl)-N-methylcyclopropane-1-carboxamide (67.5 mg, 2.0 eq.) in DMF (2 mL) were added 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (110 mg, 1.0 eq.), DIEA (128 mg, 5.0 eq.) and PyBOP (155 mg, 1.5 eq.). The reaction was stirred at 40 °C for 1 hour. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 46%-76% B over 15 min] to afford the title compound (50.0 mg, 36% yield) as yellow solid; LCMS (ESI, M+1): m/z=707.3.

Step G. trans-N-acetyl-2-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclopropane-1-carboxamide: To a solution of trans-N-acetyl-2-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N-methylcyclopropane-1-carboxamide (45.0 mg, 1.0 eq.) in DCM (6 mL) was added TFA (2 mL). The reaction was stirred at 25 °C for 1 hour. The mixture was basified with DIEA at −40 °C, diluted with water (10 mL) and extracted with DCM (2×10 mL). The combined organic layers were washed with brine (10 mL), dried over anhydrous sodium sulfate, concentrated and purified with prep-HPLC [column: Welch Xtimate C18 150×25 mm×5 μm; mobile phase: water (FA)-ACN; gradient: 20%-40% B over 10 min] to afford the title compound (21.2 mg, 47% yield, 0.21HCOOH) as white solid; ¹H NMR (400 MHz, METHANOL-d₄) δ=9.21 (d, J=0.8 Hz, 1H), 7.68 (dd, J=6.0, 9.2 Hz, 1H), 7.30 (d, J=2.8 Hz, 1H), 7.25 (t, J=9.6 Hz, 1H), 7.05 (s, 1H), 5.45-5.27 (m, 1H), 4.46-4.33 (m, 2H), 4.01-3.92 (m, 1H), 3.53-3.34 (m, 4H), 3.28 (d, J=1.2 Hz, 3H), 3.14-3.08 (m, 1H), 2.56-2.51 (m, 1H), 2.47-2.42 (m, 1H), 2.39 (s, 3H), 2.35-2.02 (m, 6H), 2.01-1.87 (m, 2H), 1.43 (td, J=4.4, 8.8 Hz, 1H), 1.15-1.11 (m, 1H), 0.79 (dt, J=4.0, 7.2 Hz, 3H); LCMS (ESI, M+1): m/z=663.3.

### Example 282

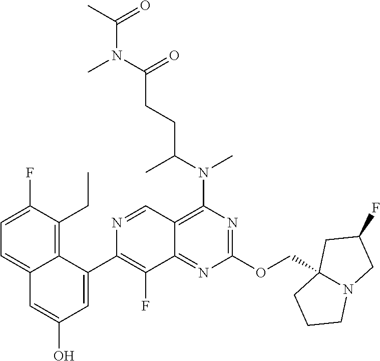 [SMILES: CCc1c(F)ccc2cc(O)cc(-c3ncc4c(N(C)C(C)CCC(=O)N(C)C(C)=O)nc(OC[C@@]56CCCN5C[C@H](F)C6)nc4c3F)c12]

### N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpentanamide

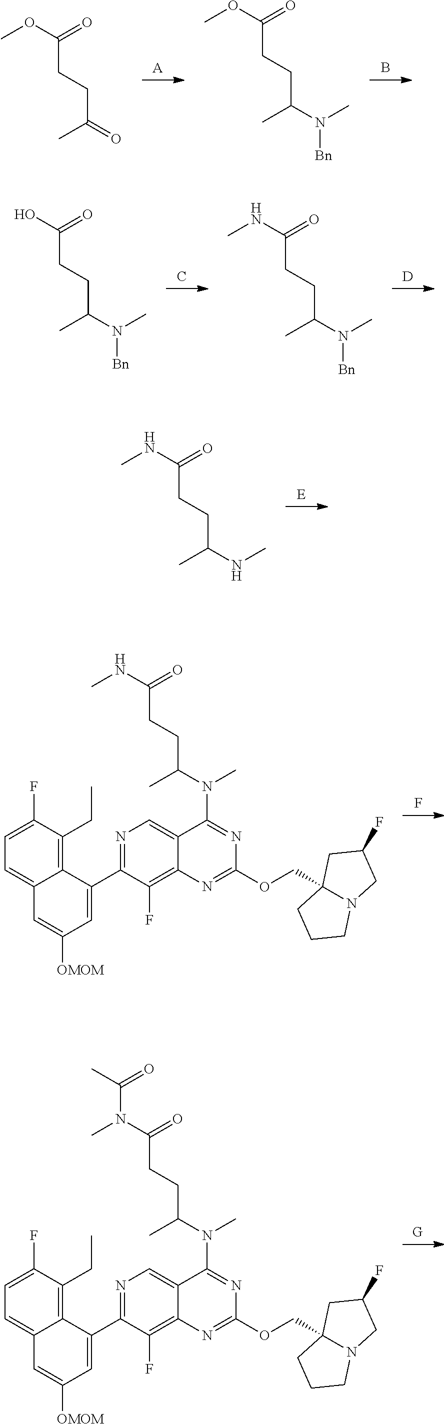

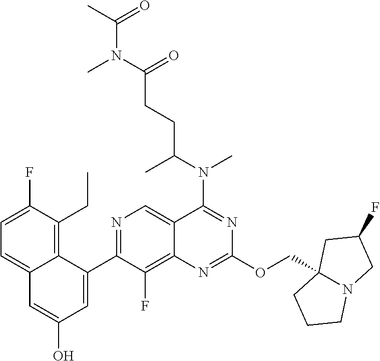 [SMILES: CCc1c(F)ccc2cc(O)cc(-c3ncc4c(N(C)C(C)CCC(=O)N(C)C(C)=O)nc(OC[C@@]56CCCN5C[C@H](F)C6)nc4c3F)c12]

Step A. methyl 4-(benzyl(methyl)amino)pentanoate: To a solution of N-methyl-1-phenylmethanamine (9.31 g, 1.0 eq.) in MeOH (150 mL) were added methyl 4-oxopentanoate (10.0 g, 1.0 eq.) and HOAc (1.32 mL, 0.3 eq.). The reaction was stirred at 42 °C for 1 hour. Then NaBH₃CN (9.66 g, 2.0 eq.) was added, and the reaction was stirred at 42 °C for 11 hours. The mixture was quenched with saturated NH₄Cl solution (100 mL) and extracted with ethyl acetate (2×100 mL). The combined organic layers were washed with brine (100 mL), dried over anhydrous sodium sulfate, concentrated, and purified with prep-TLC [SiO₂, petroleum ether/ethyl acetate=10/1 to 10/1] to afford the title compound (7.30 g, 37% yield) as yellow oil; LCMS (ESI, M+1): m/z=236.1.

Step B. 4-(benzyl(methyl)amino)pentanoic acid: To a solution of methyl 4-(benzyl(methyl)amino)pentanoate (3.00 g, 1.0 eq.) in MeOH (30 mL) was added LiOH·H₂O (2M, 10 mL). The reaction was stirred at 25 °C for 1 hour. The mixture was concentrated and purified with prep-HPLC [column: Kromasil Eternity XT 250×80 mm×10 μm; mobile phase: [water (NH₃H₂O)-ACN]; gradient: 5%-35% B over 30 min] to afford the title compound (1.65 g, 58% yield) as white solid; LCMS (ESI, M+1): m/z=222.1.

Step C. 4-(benzyl(methyl)amino)-N-methylpentanamide: To a solution of 4-(benzyl(methyl)amino)pentanoic acid (1.50 g, 1.0 eq.) and methylamine hydrochloride (915 mg, 2.0 eq.) in DMF (15 mL) were added HATU (3.87 g, 1.5 eq.) and DIEA (4.38 g, 5.0 eq.). The reaction was stirred at 25 °C for 1 hour. The mixture was diluted with water (30 mL) and extracted with ethyl acetate (2×30 mL). The combined organic layers were washed with brine (30 mL), dried over anhydrous sodium sulfate, concentrated and purified with prep-HPLC [column: Kromasil Eternity XT 250×80 mm×10 μm; mobile phase: [water (NH₄HCO₃)-ACN]; gradient: 1%-22% B over 30 min] to afford the title compound (550 mg, 34% yield) as yellow oil; LCMS (ESI, M+1): m/z=235.2.

Step D. N-methyl-4-(methylamino)pentanamide: To a solution of 4-(benzyl(methyl)amino)-N-methylpentanamide (548 mg, 1.0 eq.) in MeOH (10 mL) was added Pd/C (498 mg, 0.2 equiv, 10% purity) under N₂ atmosphere. The reaction was degassed and purged with H₂ for 3 times. The reaction was stirred under H₂ (15 psi) pressure at 40 °C for 4 hours. The mixture was filtered and concentrated to afford the title compound (300 mg, 89% yield) as yellow oil; ¹H NMR (400 MHz, METHANOL-d₄) δ=2.71 (s, 3H), 2.60-2.49 (m, 1H), 2.35 (s, 3H), 2.27-2.13 (m, 2H), 1.91-1.75 (m, 1H), 1.63-1.48 (m, 1H), 1.07 (d, J=6.4 Hz, 3H).

Step E. 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpentanamide: To a solution of N-methyl-4-mg, (methylamino)pentanamide (297 mg, 1.9 eq.) and 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (600 mg, 1.0 eq.) in DMF (8 mL) were added PyBOP (845 mg, 1.5 eq.) and DIEA (699 mg, 5.0 eq.). The reaction was stirred at 25 °C for 1 hour. The mixture was diluted with water (20 mL) and extracted with ethyl acetate (2×20 mL). The combined organic layers were washed with brine (20 mL), dried over anhydrous sodium sulfate, concentrated and purified with prep-HPLC [column: Waters Xbridge BEH C18 250×50 mm×10 μm; mobile phase: [water (NH₄HCO₃)-ACN]; gradient: 35%-65% B over 20 min] to afford the title compound (500 mg, 67% yield) as yellow oil; LCMS (ESI, M+1): m/z=681.4.

Step F. N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpentanamide: To a solution of 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpentanamide (300 mg, 1.0 eq.) in 2-MeTHF (4 mL) were added DIEA (285 mg, 5.0 eq.) and acetyl chloride (69.2 mg, 2.0 eq.). The reaction was stirred at 80 °C for 1 hour. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge Prep OBD C18 150×40 mm×10 μm; mobile phase: [water (NH₄HCO₃)-ACN]; gradient: 50%-80% B over 15 min] to afford the title compound (180 mg, 55% yield) as off-white solid; LCMS (ESI, M+1): m/z=723.4.

Step G. N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpentanamide: To a solution of N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N-methylpentanamide (20.0 mg, 1.0 eq.) in DCM (0.3 mL) was added TFA (0.1 mL). The reaction was stirred at 25 °C for 0.5 hour. The mixture was concentrated, dissolved in ethyl acetate (2 mL), basified with DIEA at −40 °C and filtered. The filtrate was concentrated and purified with prep-HPLC [column: Phenomenex luna C18 150×25 mm×10 μm; mobile phase: [water (FA)-ACN]; gradient: 18%-48% B over 9 min] to afford the title compound (7.30 mg, 36% yield, 0.2HCOOH) as white solid; ¹H NMR (400 MHz, DMSO-d₆) δ=9.93 (s, 1H), 9.24 (s, 1H), 7.76 (dd, J=6.0, 9.2 Hz, 1H), 7.39-7.30 (m, 2H), 7.05-6.96 (m, 1H), 5.40-5.18 (m, 1H), 5.06 (br s, 1H), 4.19-4.00 (m, 2H), 3.34 (br s, 3H), 3.15-3.07 (m, 2H), 3.04 (d, J=6.8 Hz, 4H), 2.89-2.72 (m, 3H), 2.40-2.32 (m, 1H), 2.26 (d, J=11.2 Hz, 3H), 2.20-1.99 (m, 5H), 1.90-1.73 (m, 4H), 1.33 (br d, J=5.2 Hz, 3H), 0.79-0.69 (m, 3H); LCMS (ESI, M+1): m/z=679.4.

### Example 283

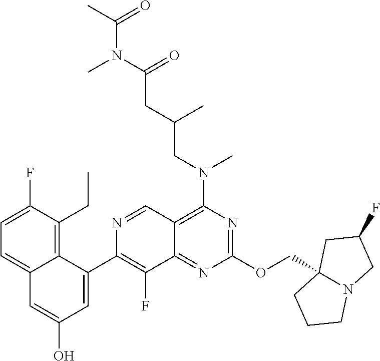 [SMILES: CCc1c(F)ccc2cc(O)cc(-c3ncc4c(N(C)CC(C)CC(=O)N(C)C(C)=O)nc(OC[C@@]56CCCN5C[C@H](F)C6)nc4c3F)c12]

### N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,3-dimethylbutanamide

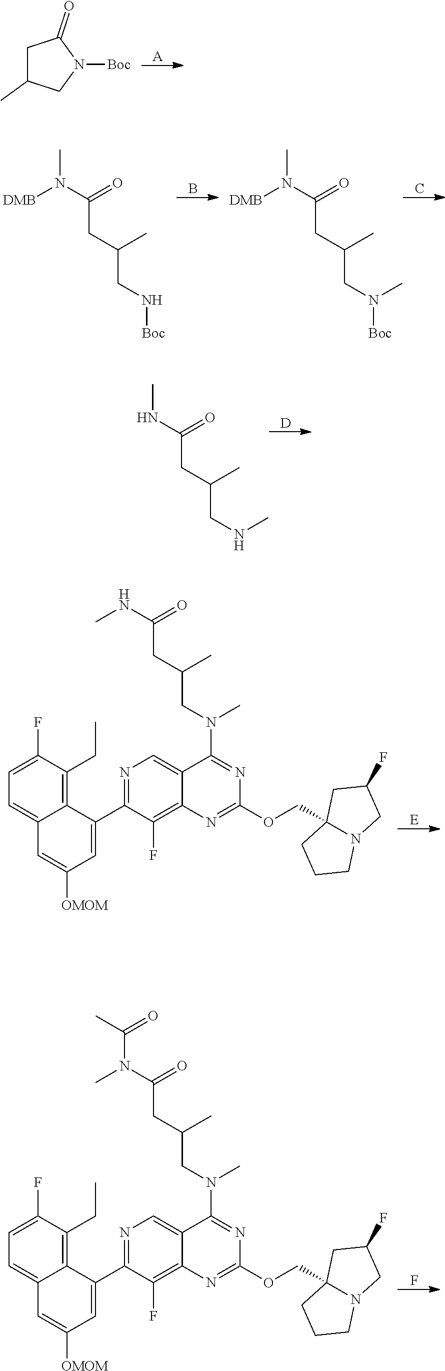

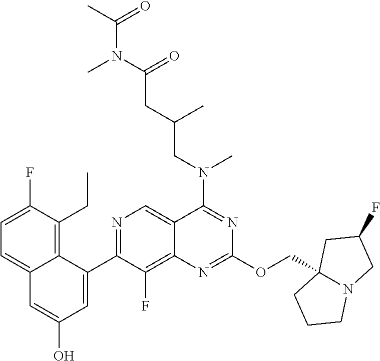 [SMILES: CCc1c(F)ccc2cc(O)cc(-c3ncc4c(N(C)CC(C)CC(=O)N(C)C(C)=O)nc(OC[C@@]56CCCN5C[C@H](F)C6)nc4c3F)c12]

Step A. tert-butyl (4-((3,4-dimethoxybenzyl)(methyl)amino)-2-methyl-4-oxobutyl)carbamate: A solution of tert-butyl 4-methyl-2-oxopyrrolidine-1-carboxylate (1.30 g, 1.0 eq.) in 1-(3,4-dimethoxyphenyl)-N-methylmethanamine (4 mL) was stirred at 120 °C for 10 hours. The mixture was diluted with water (20 mL) and extracted with ethyl acetate (3× 20 mL). The combined organic layers were washed with brine (20 mL), dried over anhydrous sodium sulfate, concentrated and purified with HPLC [0.1% FA condition] to afford the title compound (2.30 g, 89% yield) as brown oil; LCMS (ESI, M+1): m/z=381.2.

Step B. tert-butyl (4-(3,4-dimethoxybenzyl)(methyl)amino)-2-methyl-4-oxobutyl)(methyl)carbamate: To a solution of tert-butyl (4-((3,4-dimethoxybenzyl)(methyl)amino)-2-methyl-4-oxobutyl)carbamate (2.30 g, 1.0 eq.) in THF (30 mL) was added NaH (725 mg, 3.0 equiv, 60% purity) portion wise at 0 °C under N₂ atmosphere. After addition, the reaction was stirred at 0 °C for 0.5 hour. MeI (1.72 g, 2.0 eq.) was added dropwise at 0 °C under N₂ atmosphere. The reaction was stirred at 25 °C for 1 hour. The mixture was quenched with water (50 mL) and extracted with ethyl acetate (3×30 mL). The combined organic layers were washed with brine (50 mL), dried over anhydrous sodium sulfate, concentrated and purified with column chromatography [SiO₂, petroleum ether/ethyl acetate=50/1 to 20/1] to afford the title compound (2.10 g, 85% yield) as yellow oil; LCMS (ESI, M+1): m/z=395.2.

Step C. N,3-dimethyl-4-(methylamino)butanamide: To a solution of tert-butyl (4-((3,4-dimethoxybenzyl)(methyl)amino)-2-methyl-4-oxobutyl)(methyl)carbamate (1.00 g, 1.0 eq.) in DCM (6 mL) was added TFA (2 mL). The reaction was stirred at 25 °C for 1 hour. The mixture was concentrated, dissolved in MeOH (2 mL), neutralized with solid NaHCO₃, filtered and concentrated to afford the title compound (400 mg, crude) as yellow oil.

Step D. 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,3-dimethylbutanamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (700 mg, 1.0 eq.) and N,3-dimethyl-4-(methylamino)butanamide (364 mg, 2.0 eq.) in DMF (5 mL) were added PyBOP (985 mg, 1.5 eq.) and DIEA (816 mg, 5.0 eq.). The reaction was stirred at 40 °C for 1 hour. The mixture was diluted with water (20 mL) and extracted with ethyl acetate (3×20 mL). The combined organic layers were washed with brine (20 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 40%-70% B over 52 min] to afford the title compound (220 mg, 25% yield) as yellow solid; LCMS (ESI, M+1): m/z=681.5.

Step E. N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,3-dimethylbutanamide: To a solution of 4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,3-dimethylbutanamide (208 mg, 1.0 eq.) in 2-MeTHF (4 mL) were added DIEA (197 mg, 5.0 eq.) and acetyl chloride (48.0 mg, 2.0 eq.). The reaction was stirred at 80 °C for 1 hour. The mixture was diluted with water (20 mL) and extracted with ethyl acetate (3×20 mL). The combined organic layers were washed with brine (20 mL), dried over anhydrous sodium sulfate, concentrated and purified with prep-HPLC [column: Waters Xbridge Prep OBD C18 150×40 mm×10 μm; mobile phase: water (NH₄HCO₃)-ACN; gradient: 48%-78% B over 52 min] to afford the title compound (200 mg, 90% yield) as brown solid; LCMS (ESI, M+1): m/z=723.4.

Step F. N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,3-dimethylbutanamide: To a solution of N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,3-dimethylbutanamide (60.0 mg, 1.0 eq.) in DCM (3 mL) was added TFA (1 mL). The reaction was stirred at 25 °C for 1 hour. The mixture was concentrated, dissolved in MeCN (2 mL), neutralized with DIEA at −40 °C, diluted with water (10 mL) and extracted with ethyl acetate (3×10 mL). The combined organic layers were washed with brine (10 mL), dried over anhydrous sodium sulfate, concentrated and purified with prep-HPLC [column: Waters Xbridge 150×25 mm×5 μm; mobile phase: water (NH₄HCO₃)-ACN; gradient: 45%-75% B over 9 min] to afford the title compound (7.43 mg, 12% yield) as white solid; ¹H NMR (400 MHz, DMSO-d₆) δ=9.90 (s, 1H), 9.30 (s, 1H), 7.76 (dd, J=6.0, 8.8 Hz, 1H), 7.40-7.29 (m, 2H), 7.01 (s, 1H), 5.39-5.16 (m, 1H), 4.17-4.10 (m, 1H), 4.09-3.69 (m, 3H), 3.59 (d, J=2.4 Hz, 3H), 3.13-3.00 (m, 6H), 2.97-2.79 (m, 2H), 2.72-2.61 (m, 2H), 2.40-2.32 (m, 1H), 2.27 (d, J=1.2 Hz, 3H), 2.15 (br dd, J=10.4, 15.2 Hz, 2H), 2.08-1.95 (m, 2H), 1.89-1.73 (m, 3H), 1.03-0.97 (m, 3H), 0.74 (t, J=7.2 Hz, 3H); LCMS (ESI, M+1): m/z=679.5.

### Example 284

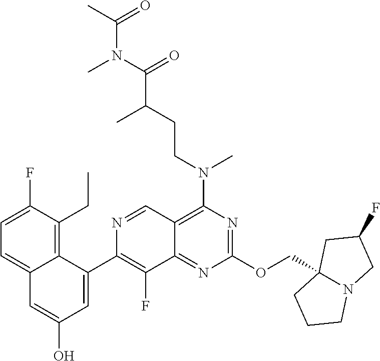 [SMILES: CCc1c(F)ccc2cc(O)cc(-c3ncc4c(N(C)CCC(C)C(=O)N(C)C(C)=O)nc(OC[C@@]56CCCN5C[C@H](F)C6)nc4c3F)c12]

### N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,2-dimethylbutanamide

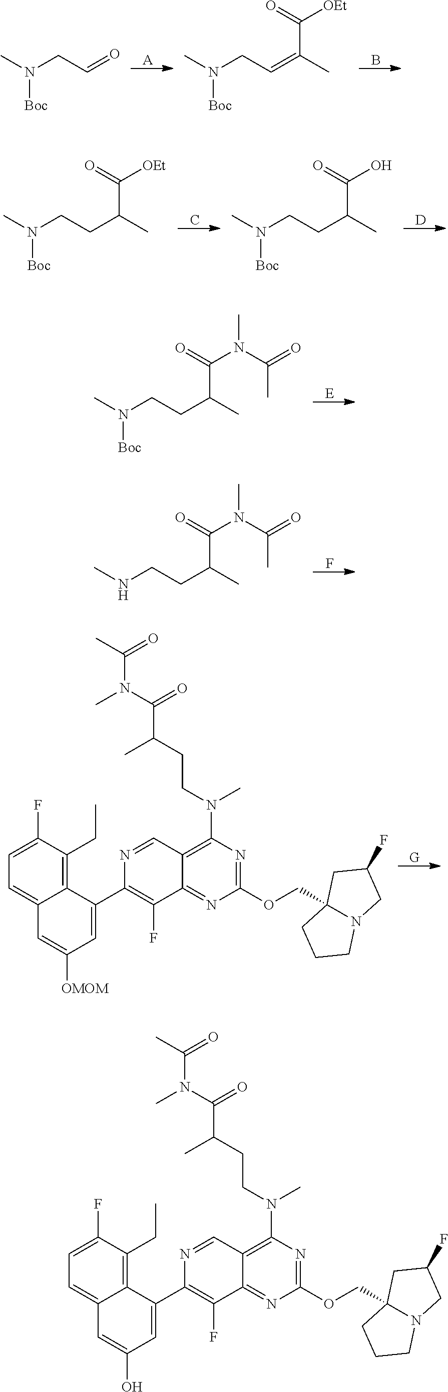

Step A. ethyl (Z)-4-((tert-butoxycarbonyl)(methyl)amino)-2-methylbut-2-enoate: To a solution of ethyl 2-(diethoxyphosphoryl) propanoate (6.29 g, 1.0 eq.) in THF (120 mL) was added sodium hydride (1.39 g, 1.2 equiv, 60% purity) under N₂ atmosphere at 0 °C. The reaction was stirred at 0 °C for 0.5 hour and a solution of tert-butyl methyl(2-oxoethyl)carbamate (5.00 g, 1.5 eq.) in THF (5 mL) was added at 0 °C. The reaction was stirred at 0 °C for 1 hour under nitrogen atmosphere. The mixture was quenched with water (100 mL) at 0 °C and extracted with ethyl acetate (2×80 mL). The combined organic layers were washed with brine (100 mL), dried over anhydrous sodium sulfate and concentrated to afford the title compound (5.83 g, crude) as colorless oil; ¹H NMR (400 MHz, CHLOROFORM-d) δ=6.64 (dt, J=1.2, 6.4 Hz, 1H), 4.19 (dd, J=1.2, 6.8 Hz, 2H), 3.97 (br s, 2H), 2.83 (br s, 3H), 1.86 (s, 3H), 1.44 (s, 9H), 1.29-1.27 (m, 3H).

Step B. ethyl 4-((tert-butoxycarbonyl)(methyl)amino)-2-methylbutanoate: To a solution of ethyl (Z)-4-((tert-butoxycarbonyl)(methyl)amino)-2-methylbut-2-enoate (2.83 g, 1.0 eq.) in EtOH (30 mL) was added Pd/C (1.17 g, 0.1 equiv, 10% purity) under nitrogen atmosphere. The reaction was degassed and purged with hydrogen for 3 times. The reaction was stirred at 40 °C for 4 hours under hydrogen (15 Psi) atmosphere. The mixture was filtered and concentrated to afford the title compound (2.39 g, crude) as colorless oil; ¹H NMR (400 MHz, CHLOROFORM-d) δ=4.06 (q, J=6.8 Hz, 2H), 3.15 (br s, 2H), 2.76 (s, 3H), 2.35 (sxt, J=6.8 Hz, 1H), 1.92-1.77 (m, 1H), 1.52 (br dd, J=6.3, 12.9 Hz, 1H), 1.38 (s, 9H), 1.19 (t, J=7.2 Hz, 3H), 1.12 (d, J=6.8 Hz, 3H).

Step C. 4-((tert-butoxycarbonyl)(methyl)amino)-2-methylbutanoic acid: To a solution of ethyl 4-((tert-butoxycarbonyl)(methyl)amino)-2-methylbutanoate (1.17 g, 1.0 eq.) in MeOH (6 mL) was added LiOH·H₂O (2M, 3 mL). The reaction was stirred at 25 °C for 12 hours. The mixture was diluted with water (30 mL) and extracted with ethyl acetate (2×10 mL). The aqueous phase was acidified with solid citric acid (pH ~5) and extracted with ethyl acetate (2×20 mL). The combined organic layers were washed with brine (20 mL), dried over anhydrous sodium sulfate and concentrated to afford the title compound (920 mg, crude) as colorless oil; LCMS (ESI, M+23): m/z=254.1.

Step D. tert-butyl methyl (3-methyl-4-(N-methylacetamido)-4-oxobutyl)carbamate: To a solution of 4-((tert-butoxycarbonyl)(methyl)amino)-2-methylbutanoic acid (920 mg, 1.0 eq.) in 2-methyltetrahydrofuran (20 mL) were added DIEA (2.57 g, 5.0 eq.), N-methylacetamide (1.45 g, 5.0 eq.) and CMPI (5.08 g, 5.0 eq.). The reaction was stirred at 80 °C for 1 hour. The mixture was diluted with water (30 mL) and extracted with ethyl acetate (2×30 mL). The combined organic layers were washed with brine (30 mL), dried over anhydrous sodium sulfate, concentrated and purified with prep-HPLC [column: Phenomenex luna C18 150×40 mm×15 μm; mobile phase: water (FA)-ACN; gradient: 35%-65% B over 15 min] to afford the title compound (815 mg, 61% yield) as black brown oil; LCMS (ESI, M+23): m/z=309.1.

Step E. N-acetyl-N,2-dimethyl-4-(methylamino)butanamide: To a solution of tert-butyl methyl (3-methyl-4-(N-methylacetamido)-4-oxobutyl)carbamate (760 mg, 1.0 eq.) in DCM (6 mL) was added TFA (2 mL). The reaction was stirred at 0 °C for 0.5 hour. The mixture was concentrated at 10 °C, diluted with DCM (5 mL), neutralized with DIEA at −40 °C and concentrated to afford the title compound (494 mg, crude) as black-brown oil; LCMS (ESI, M+1): m/z=187.1.

Step F. N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,2-dimethylbutanamide: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (495 mg, 1.0 eq.) in DCM (10 mL) were added DIEA (346 mg, 3.0 eq.), DMAP (21.8 mg, 0.2 eq.) and 2-nitrobenzenesulfonyl chloride (297 mg, 1.5 eq.) at 0 °C. The reaction was stirred at 25 °C for 1 hour. Then N-acetyl-N,2-dimethyl-4-(methylamino)butanamide (494 mg, 3.0 eq.) was added. The reaction was stirred at 25 °C for 0.5 hour. The mixture was filtered. The filtrate was concentrated and purified with prep-HPLC [column: Phenomenex luna C18 150×50 mmx 10 μm; mobile phase: water (FA)-ACN; gradient: 30%-60% B over 18 min] to afford the title compound (150 mg, 19% yield) as brown solid; LCMS (ESI, M+1): m/z=723.4.

Step G. N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,2-dimethylbutanamide: To a solution of N-acetyl-4-((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)-N,2-dimethylbutanamide (100 mg, 1.0 eq.) in DCM (0.9 mL) was added TFA (0.3 mL). The reaction was stirred at 25 °C for 1 hour. The mixture was concentrated, dissolved in ethyl acetate (5 mL) and neutralized with DIEA at −40 °C. The mixture was diluted water (5 mL) and extracted with ethyl acetate (3×5 mL). The combined organic layers were washed with brine (5 mL), dried over anhydrous sodium sulfate, concentrated and purified with prep-HPLC [column: Waters Xbridge 150×25 mm×5 μm; mobile phase: water (NH₄HCO₃)-ACN; gradient: 40%-70% B over 10 min] to afford the title compound (5.42 mg, 5.7% yield) as white solid; ¹H NMR (400 MHz, DMSO-d₆) δ=10.01-9.86 (m, 1H), 9.24 (s, 1H), 7.76 (dd, J=6.0, 8.8 Hz, 1H), 7.38-7.31 (m, 2H), 7.01 (d, J=2.4 Hz, 1H), 5.37-5.17 (m, 1H), 4.16-4.10 (m, 1H), 4.07-4.02 (m, 1H), 3.93-3.83 (m, 2H), 3.51 (s, 3H), 3.43-3.35 (m, 2H), 3.10-3.06 (m, 4H), 3.01 (s, 1H), 2.86-2.79 (m, 1H), 2.40-2.29 (m, 2H), 2.24 (s, 3H), 2.17-2.03 (m, 4H), 1.87-1.74 (m, 4H), 1.17 (d, J=6.4 Hz, 3H), 0.73 (t, J=7.2 Hz, 3H); LCMS (ESI, M+1): m/z=679.3.

### Example 285

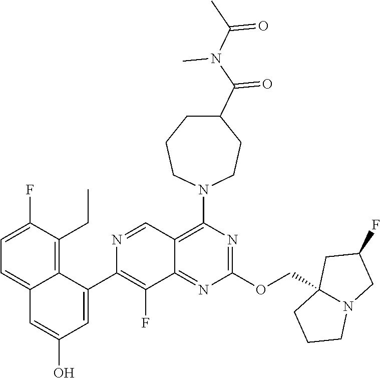 [SMILES: CCc1c(F)ccc2cc(O)cc(-c3ncc4c(N5CCCC(C(=O)N(C)C(C)=O)CC5)nc(OC[C@@]56CCCN5C[C@H](F)C6)nc4c3F)c12]

### N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazepane-4-carboxamide

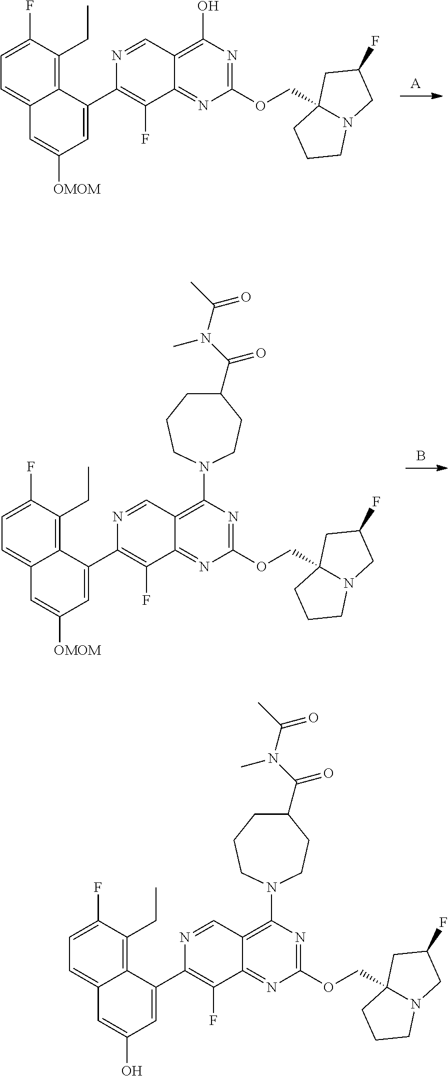

Step A. N-acetyl-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazepane-4-carboxamide: To a mixture of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (100 mg, 1.0 eq.), DIEA (58.3 mg, 2.5 eq.) and DMAP (1.10 mg, 0.05 eq.) in DCM (1.00 mL) was added 2-nitrobenzenesulfonyl chloride (44.0 mg, 1.1 eq.) at 0 °C. The reaction was stirred at 0 °C for 0.5 hour. And then N-acetyl-N-methylazepane-4-carboxamide (67.6 mg, 1.2 equiv, TFA) was added to above mixture. The reaction was stirred at 20 °C for 12 hours. The mixture was diluted with water (4 mL) and extracted with DCM (3×3 mL). The combined organic layers were dried over anhydrous sodium sulfate, concentrated, and purified with reversed phase flash chromatography [C18, 0.1% formic acid condition] to afford the title compound (75.0 mg, 54% yield) as yellow solid; LCMS (ESI, M+1): m/z=735.4.

Step B. N-acetyl-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazepane-4-carboxamide: To a solution of N-acetyl-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methylazepane-4-carboxamide (70.0 mg, 1.0 eq.) in DCM (0.2 mL) was added TFA (768 mg, 71 eq.). The reaction was stirred at 20 °C for 0.5 hour. The mixture was basified with saturated NaHCO₃ aqueous solution (10 mL) and extracted with DCM (3×3 mL). The combined organic layers were dried over anhydrous sodium sulfate, concentrated and purified with reversed phase flash chromatography [C18, 0.1% formic acid condition] and prep-HPLC [Waters xbridge 150×25 mm 10 μm; A: water (NH₄HCO₃); B: CAN gradient: 50%-70% B over 8 min] to afford the title compound (4.37 mg, 6.6% yield) as white solid; ¹H NMR (400 MHz, METHANOL-d₄) δ=9.15 (d, J=4.4 Hz, 1H), 7.68 (dd, J=5.6, 9.2 Hz, 1H), 7.30 (d, J=2.4 Hz, 1H), 7.25 (t, J=9.2 Hz, 1H), 7.07 (dd, J=2.4, 8.8 Hz, 1H), 5.38-5.23 (m, 1H), 4.43-4.28 (m, 3H), 4.26-3.84 (m, 2H), 3.51-3.36 (m, 2H), 3.25-3.17 (m, 6H), 3.05-2.97 (m, 1H), 2.57-2.41 (m, 1H), 2.34 (d, J=2.4 Hz, 3H), 2.32-1.94 (m, 11H), 1.92-1.84 (m, 1H), 1.77-1.64 (m, 1H), 0.88-0.72 (m, 3H); SFC [Column: Chiralpak IC-3 50×4.6 mm I.D., 3 μm; Mobile phase: Phase A for CO₂, and Phase B for IPA+ACN (0.05% DEA); Gradient elution: 40% IPA+ACN (0.05% DEA) in CO₂; Flow rate: 3 mL/min; Detector: PDA; Column Temp: 35 °C; Back Pressure: 100 Bar]; LCMS (ESI, M+1): m/z=691.4; RT=1.090 minutes.

### Example 286

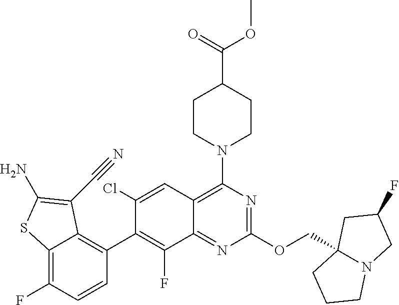 [SMILES: COC(=O)C1CCN(c2nc(OC[C@@]34CCCN3C[C@H](F)C4)nc3c(F)c(-c4ccc(F)c5sc(N)c(C#N)c45)c(Cl)cc23)CC1]

### Methyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

See Example 129 step B for the synthesis. The title compound was obtained as yellow solid; ¹H NMR (400 MHz, DIMETHYL SULFOXIDE-d₆) δ=8.10 (br s, 2H), 7.82 (s, 1H), 7.26 (dd, J=5.2, 8.4 Hz, 1H), 7.19-7.10 (m, 1H), 5.38-5.13 (m, 1H), 4.22 (br t, J=14.2 Hz, 2H), 4.13-4.06 (m, 1H), 4.04-3.97 (m, 1H), 3.64 (s, 3H), 3.42-3.32 (m, 2H), 3.14-2.97 (m, 3H), 2.88-2.71 (m, 2H), 2.16-2.10 (m, 1H), 2.03 (br d, J=16.4 Hz, 4H), 1.89-1.71 (m, 5H); LCMS (ESI, M+1): m/z=671.1.

### Example 287

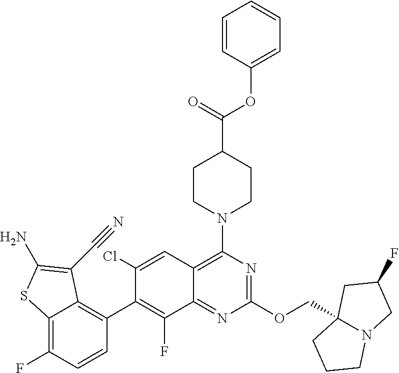 [SMILES: N#Cc1c(N)sc2c(F)ccc(-c3c(Cl)cc4c(N5CCC(C(=O)Oc6ccccc6)CC5)nc(OC[C@@]56CCCN5C[C@H](F)C6)nc4c3F)c12]

### phenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

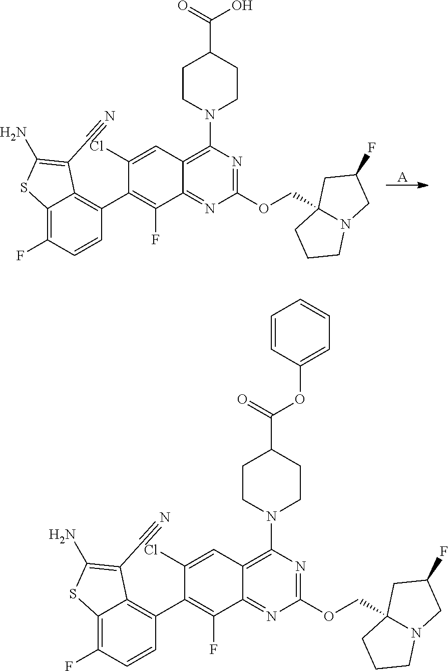

Step A. phenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (60 mg, 1.0 eq.) and phenol (12.9 mg, 1.5 eq.) in DMF (1 mL) were added EDCI (26.3 mg, 1.5 eq.) and DMAP (16.7 mg, 1.5 eq.) at 25 °C. The reaction was stirred at 60 °C for 2 hours. The mixture was diluted with water (4 mL) and extracted with ethyl acetate (3×3 mL). The organic layer was dried over anhydrous sodium sulfate, concentrated, and purified with prep-HPLC [Waters Xbridge 150×25 mm×5 μm; A: water (NH₄HCO₃); B: ACN, B %: 54%-84% over 15 min] to afford the title compound (5.96 mg, 8% yield) as a yellow solid. ¹H NMR (400 MHz, CHLOROFORM-d₃) δ=7.74 (s, 1H), 7.46-7.37 (m, 2H), 7.24-7.20 (m, 1H), 7.15-6.97 (m, 4H), 5.40-5.22 (m, 3H), 4.41-4.17 (m, 4H), 3.46-3.30 (m, 3H), 3.28-3.16 (m, 2H), 3.05-2.88 (m, 2H), 2.37-2.24 (m, 4H), 2.20-2.07 (m, 3H), 2.02-1.86 (m, 3H); LCMS (ESI, M+1): m/z=733.3.

### Example 288

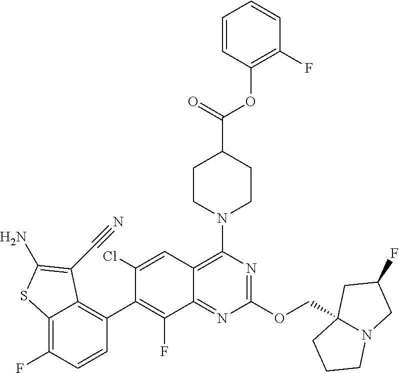 [SMILES: N#Cc1c(N)sc2c(F)ccc(-c3c(Cl)cc4c(N5CCC(C(=O)Oc6ccccc6F)CC5)nc(OC[C@@]56CCCN5C[C@H](F)C6)nc4c3F)c12]

### 2-fluorophenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 287. The title compound was obtained as yellow solid; ¹H NMR (400 MHz, CHLOROFORM-d₃) δ=7.74 (s, 1H), 7.25-7.21 (m, 2H), 7.20-7.14 (m, 3H), 7.09-6.99 (m, 1H), 5.37 (br d, J=4.4 Hz, 2H), 5.35-5.17 (m, 1H), 4.41-4.17 (m, 4H), 3.50-3.37 (m, 2H), 3.35-3.23 (m, 2H), 3.22-3.12 (m, 1H), 3.07-2.93 (m, 2H), 2.37-2.27 (m, 3H), 2.24-2.09 (m, 4H), 2.01-1.88 (m, 3H); LCMS (ESI, M+1): m/z=751.2.

### Example 289

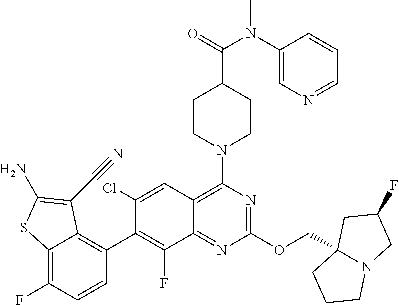 [SMILES: CN(C(=O)C1CCN(c2nc(OC[C@@]34CCCN3C[C@H](F)C4)nc3c(F)c(-c4ccc(F)c5sc(N)c(C#N)c45)c(Cl)cc23)CC1)c1cccnc1]

### 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methyl-N-(pyridin-3-yl)piperidine-4-carboxamide

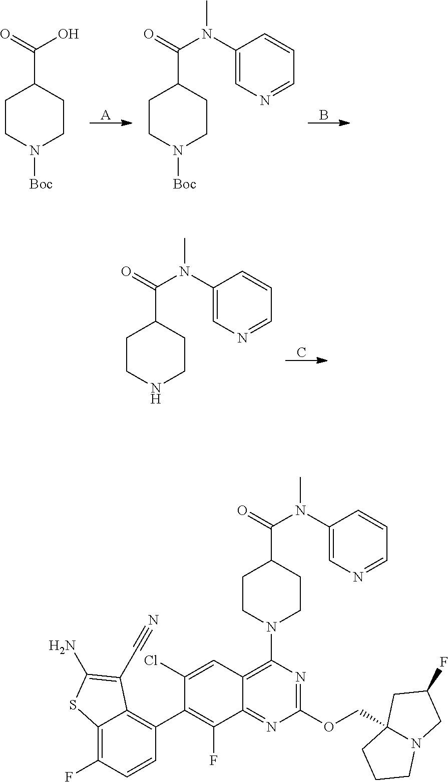

Step A. tert-butyl 4-(methyl(pyridin-3-yl)carbamoyl)piperidine-1-carboxylate: To a solution of 1-(tert-butoxycarbonyl)piperidine-4-carboxylic acid (100 mg, 1.0 eq.) and N-methylpyridin-3-amine (141 mg, 3.0 eq.) in 2-MeTHF (1.0 mL) were added DIEA (564 mg, 10 eq.) and CMPI (446 mg, 4.0 eq.). The reaction was stirred at 80 °C for 1 hour. The mixture was diluted with water (3 mL) and extracted with ethyl acetate (3×2 mL). The combined organic layers were dried over anhydrous sodium sulfate, concentrated and purified with reversed phase flash chromatography [C18, 0.1% formic acid condition] to afford the title compound (120 mg, 77% yield) as yellow solid; LCMS (ESI, M+23): m/z=342.1.

Step B. N-methyl-N-(pyridin-3-yl)piperidine-4-carboxamide: To a solution of tert-butyl 4-(methyl(pyridin-3-yl)carbamoyl)piperidine-1-carboxylate (120 mg, 1.0 eq.) in DCM (1 mL) was added TFA (1.54 g, 36 eq.). The reaction was stirred at 25 °C for 15 minutes. The mixture was concentrated to afford the title compound (90 mg, crude, TFA salt) as yellow solid.

Step C. 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methyl-N-(pyridin-3-yl)piperidine-4-carboxamide: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (100 mg, 1.0 eq.) and N-methyl-N-(pyridin-3-yl)piperidine-4-carboxamide (90 mg, 1.5 equiv, TFA salt) in DMF (1 mL) were added PyBOP (143 mg, 1.5 eq.) and DIEA (237 mg, 10 eq.). The reaction was stirred at 25 °C for 12 hours. The mixture was filtered and the filtrate was purified with prep-HPLC [Waters Xbridge 150×25 mm×5 μm; A: water (NH₄HCO₃), B: ACN, B %: 42%-72% over 9 min] to afford the title compound (55.7 mg, 40% yield) as off-white solid; ¹H NMR (400 MHz, CHLOROFORM-d₃) δ=8.73-8.63 (m, 1H), 8.58 (d, J=2.4 Hz, 1H), 7.69 (s, 1H), 7.65-7.58 (m, 1H), 7.50-7.39 (m, 1H), 7.23-7.17 (m, 1H), 7.03 (t, J=8.8 Hz, 1H), 5.41 (s, 2H), 5.34-5.14 (m, 1H), 4.40-4.27 (m, 2H), 4.26-4.16 (m, 1H), 4.15-4.06 (m, 1H), 3.33 (br s, 3H), 3.25-3.10 (m, 3H), 3.03-2.85 (m, 3H), 2.58-2.41 (m, 1H), 2.30-2.03 (m, 5H), 1.98-1.73 (m, 5H); LCMS (ESI, M+1): m/z=747.2.

### Example 290

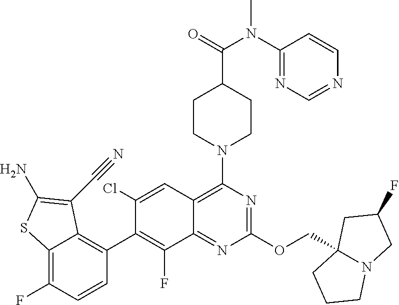 [SMILES: CN(C(=O)C1CCN(c2nc(OC[C@@]34CCCN3C[C@H](F)C4)nc3c(F)c(-c4ccc(F)c5sc(N)c(C#N)c45)c(Cl)cc23)CC1)c1ccncn1]

### 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-methyl-N-(pyrimidin-4-yl)piperidine-4-carboxamide

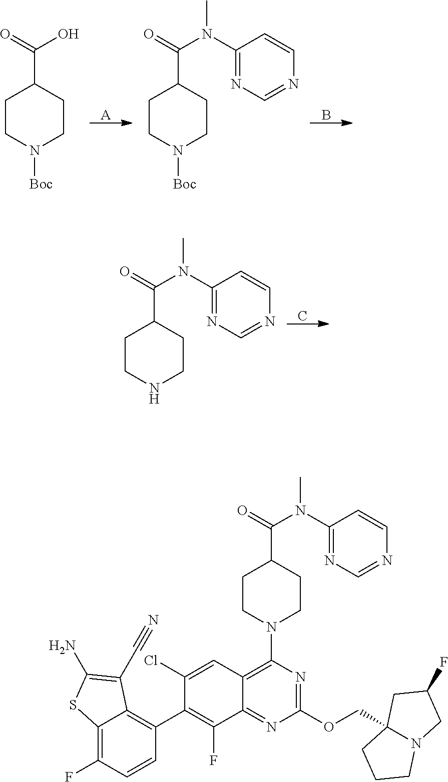

Synthesized according to the method used for Example 289. The title compound was obtained as yellow solid; ¹H NMR (400 MHz, CHLOROFORM-d₃) δ=9.03 (s, 1H), 8.68 (d, J=6.0 Hz, 1H), 7.74 (d, J=1.2 Hz, 1H), 7.62 (d, J=6.0 Hz, 1H), 7.22 (dd, J=5.2, 8.3 Hz, 1H), 7.08-7.01 (m, 1H), 5.51-5.16 (m, 3H), 4.46-4.36 (m, 2H), 4.31-4.22 (m, 1H), 4.19-4.11 (m, 1H), 3.55 (s, 3H), 3.36-3.13 (m, 6H), 3.02-2.93 (m, 1H), 2.31-2.26 (m, 1H), 2.22-2.04 (m, 6H), 1.96-1.85 (m, 3H); LCMS (ESI, M+1): m/z=748.2.

### Example 291

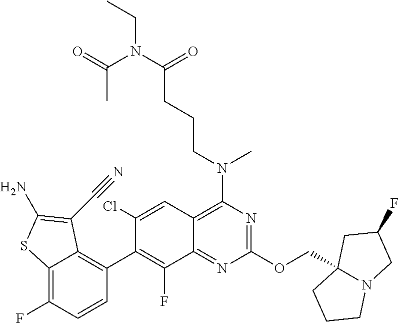 [SMILES: CCN(C(C)=O)C(=O)CCCN(C)c1nc(OC[C@@]23CCCN2C[C@H](F)C3)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-ethylbutanamide

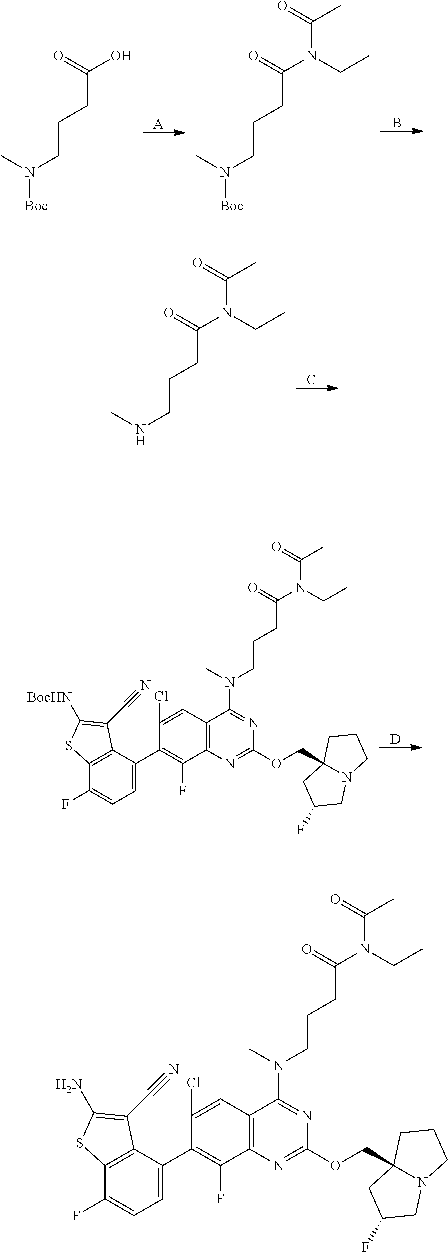

Step A. tert-butyl (4-(N-ethylacetamido)-4-oxobutyl)(methyl)carbamate: To a solution of 4-((tert-butoxycarbonyl)(methyl)amino)butanoic acid (5.00 g, 1.0 eq.) and N-ethylacetamide (10.0 g, 5.0 eq.) in 2-methyltetrahydrofuran (80 mL) were added CMPI (29.4 g, 5.0 eq.) and DIEA (14.8 g, 20 mL, 5.0 eq.) at 25 °C. The reaction was stirred at 80 °C for 1 hour. The mixture was cooled to room temperature, quenched with H₂O (100 mL) and extracted with ethyl acetate (3×100 mL). The combined organic layers were washed with brine (100 mL), dried over anhydrous sodium sulfate, concentrated and purified with column chromatography [SiO₂, petroleum ether/ethyl acetate=5/1 to 3/1] to afford the title compound (4.00 g, 61% yield) as yellow oil; ¹H NMR (400 MHz, CHLOROFORM-d) δ=3.74 (q, J=7.2 Hz, 2H), 3.29 (br t, J=6.8 Hz, 2H), 2.86 (s, 3H), 2.70 (br t, J=6.4 Hz, 2H), 2.41 (s, 3H), 1.95-1.82 (m, 2H), 1.46 (s, 9H), 1.20 (t, J=7.2 Hz, 3H)

Step B. N-acetyl-N-ethyl-4-(methylamino)butanamide: To a solution of tert-butyl (4-(N-ethylacetamido)-4-oxobutyl)(methyl)carbamate (800 mg, 1.0 eq.) in DCM (5 mL) was added TFA (24.6 g, 1.6 mL, 78.6 eq.) at 0 °C. The reaction was stirred at 0 °C for 0.5 hour. The mixture was concentrated to afford the title compound (500 mg, 96% yield) as yellow oil.

Step C. tert-butyl (4-(6-chloro-4-((4-(N-ethylacetamido)-4-oxobutyl)(methyl)amino)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (300 mg, 1.0 eq.) and N-acetyl-N-ethyl-4-(methylamino)butanamide (433 mg, 5.0 eq.) in DMF (3 mL) were added PyBOP (362 mg, 1.5 eq.) and DIEA (240. mg, 324 μL, 4.0 eq.) at 25 °C. The reaction was stirred at 25 °C for 2 hours. The mixture was added to water (10 mL) slowly, forming a suspension. It was filtered and the filter cake was collected and dried in vacuum to afford the title compound (200 mg, 53% yield) as yellow solid; LCMS (ESI, M+1): m/z=814.4.

Step D. N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-ethylbutanamide: To a solution of tert-butyl (4-(6-chloro-4-((4-(N-ethylacetamido)-4-oxobutyl)(methyl)amino)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (80.0 mg, 1.0 eq.) in DCM (1 mL) was added TFA (461 mg, 0.3 mL, 41 eq.) at 0 °C. The reaction was stirred at 0 °C for 2 hours. The mixture was concentrated and purified with prep-HPLC [Waters Xbridge 150×25 mm×5 μm; A: water (10 mM NH₄HCO₃), B: ACN, B %: 52%-82% over 9 min] to afford the title compound (25.0 mg, 35% yield) as yellow solid; ¹H NMR (400 MHz, DMSO-d₆) δ=8.09 (s, 2H), 8.08 (d, J=1.2 Hz, 1H), 7.24 (dd, J=5.2, 8.4 Hz, 1H), 7.18-7.11 (m, 1H), 5.38-5.16 (m, 1H), 4.12-4.05 (m, 1H), 4.04-3.97 (m, 1H), 3.88-3.71 (m, 2H), 3.63 (q, J=7.2 Hz, 2H), 3.41 (s, 3H), 3.15-2.97 (m, 3H), 2.86-2.76 (m, 3H), 2.32 (s, 3H), 2.18-2.10 (m, 1H), 2.09-1.98 (m, 4H), 1.87-1.72 (m, 3H), 1.05 (t, J=6.8 Hz, 3H); LCMS (ESI, M+1): m/z=714.3.

### Example 292

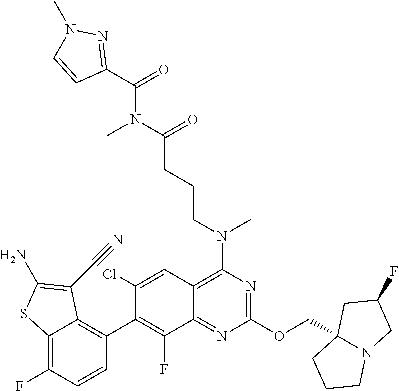 [SMILES: CN(C(=O)CCCN(C)c1nc(OC[C@@]23CCCN2C[C@H](F)C3)nc2c(F)c(-c3ccc(F)c4sc(N)c(C#N)c34)c(Cl)cc12)C(=O)c1ccn(C)n1]

### N-(4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoyl)-N, 1-dimethyl-1H-pyrazole-3-carboxamide

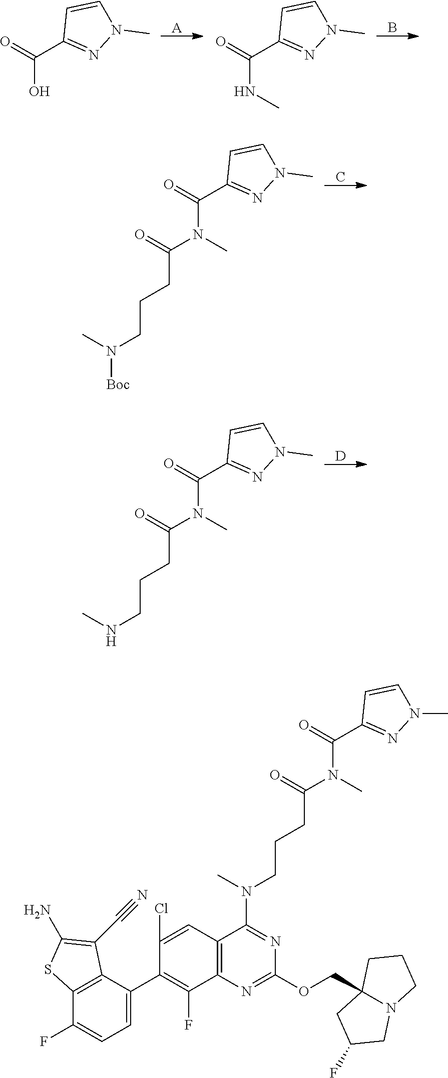

Step A. N,1-dimethyl-1H-pyrazole-3-carboxamide: To a solution of 1-methyl-1H-pyrazole-3-carboxylic acid (4.0 g, 1.0 eq.) in DMF (10 mL) were added HATU (14.5 g, 1.2 eq.), DIEA (12.3 g, 16.6 mL, 3.0 eq.) and methanamine (3 M, 31.7 mL, 3.0 eq.) at 25 °C. The reaction was stirred at 25 °C for 16 hours. The mixture was diluted with H₂O (100 mL) and extracted with ethyl acetate (3×100 mL). The combined organic layers were washed with brine, dried over anhydrous sodium sulfate, concentrated and purified with column chromatography [SiO₂, petroleum ether/ethyl acetate=2/1 to 0/1] to afford the title compound (750 mg, 15% yield) as yellow oil; ¹H NMR (400 MHz, DMSO-d₆) δ=8.01 (s, 1H), 7.74 (d, J=2.0 Hz, 1H), 6.57 (d, J=2.4 Hz, 1H), 3.88 (s, 3H), 2.69 (s, 3H).

Step B. tert-butyl (4-(N,1-dimethyl-1H-pyrazole-3-carboxamido)-4-oxobutyl)(methyl)carbamate: To a solution of N, 1-dimethyl-1H-pyrazole-3-carboxamide (750 mg, 1.0 eq.) and 4-((tert-butoxycarbonyl)(methyl)amino)butanoic acid (1.41 g, 1.2 eq.) in 2-methyltetrahydrofuran (15 mL) were added CMPI (2.75 g, 2.0 eq.) and DIEA (2.09 g, 3.0 eq.) at 25 °C. The reaction was stirred at 80 °C for 2 hours. The mixture was filtered, concentrated, and purified with column chromatography [SiO₂, petroleum ether/ethyl acetate=1/0 to 2/1] to afford the title compound (290 mg, 11% yield) as a yellow oil; LCMS (ESI, M+1, M+23): m/z=339.2, 361.3.

Step C. N,1-dimethyl-N-(4-(methylamino)butanoyl)-1H-pyrazole-3-carboxamide: To a solution of tert-butyl (4-(N,1-dimethyl-1H-pyrazole-3-carboxamido)-4-oxobutyl)(methyl)carbamate (190 mg, 1.0 eq.) in DCM (5 mL) was added TFA (2 mL) at 25 °C. The reaction was stirred at 25 °C for 2 hours. The mixture was concentrated to afford the title compound (240 mg, TFA) as a yellow oil; LCMS (ESI, M+1): m/z=239.3

Step D. N-(4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoyl)-N,1-dimethyl-1H-pyrazole-3-carboxamide: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (50.0 mg, 1.0 eq.) in DMF (2 mL) were added PyBOP (71.5 mg, 1.5 eq.), DIEA (35.5 mg, 3.0 eq.) and N, 1-dimethyl-N-(4-(methylamino)butanoyl)-1H-pyrazole-3-carboxamide (230 mg, 5.0 equiv, TFA) at 25 °C. The reaction was stirred at 25 °C for 1 hour. The mixture was diluted with water (10 mL) and extracted with ethyl acetate (3×10 mL). The combined organic phase was washed with brine (3×20 mL), dried over anhydrous sodium sulfate, and concentrated under reduced pressure to give a residue. The residue was purified with prep-HPLC [Waters Xbridge 150×25 mm×5 μm; A: water (10 mM NH₄HCO₃), B: ACN, B %: 40%-70% over 15 min] to afford the title compound (10.0 mg, 13.4% yield) as off-white solid; ¹H NMR (400 MHz, methanol-d₄) δ=8.07 (s, 1H), 7.64 (s, 1H), 7.20 (dd, J=5.2, 8.4 Hz, 1H), 7.09-6.96 (m, 1H), 6.72 (s, 1H), 5.36-5.21 (m, 1H), 4.27-4.22 (m, 1H), 4.20-4.15 (m, 1H), 3.93-3.84 (m, 5H), 3.50 (s, 3H), 3.27 (s, 3H), 3.19-3.15 (m, 2H), 3.08-2.95 (m, 2H), 2.75 (br t, J=7.2 Hz, 2H), 2.24-2.08 (m, 5H), 2.02-1.87 (m, 3H); LCMS (ESI, M+1): m/z=766.3.

### Example 293

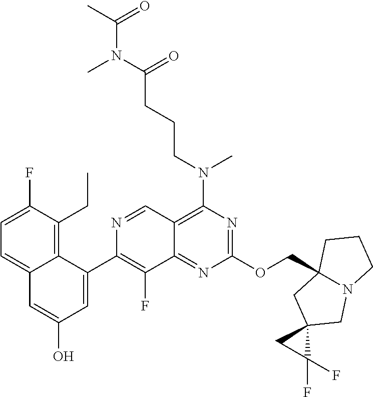 [SMILES: CCc1c(F)ccc2cc(O)cc(-c3ncc4c(N(C)CCCC(=O)N(C)C(C)=O)nc(OC[C@@]56CCCN5C[C@@]5(CC5(F)F)C6)nc4c3F)c12]

### N-acetyl-4-((2-(((1S,7a'S)-2,2-difluorodihydro-1′H,3′H-spiro[cyclopropane-1,2′-pyrrolizin]-7a′ (5′H)-yl)methoxy)-7-(8-ethyl-7-fluoro-3-hydroxynaphthalen-1-yl)-8-fluoropyrido[4,3-d]pyrimidin-4-yl)(methyl)amino)-N-methylbutanamide

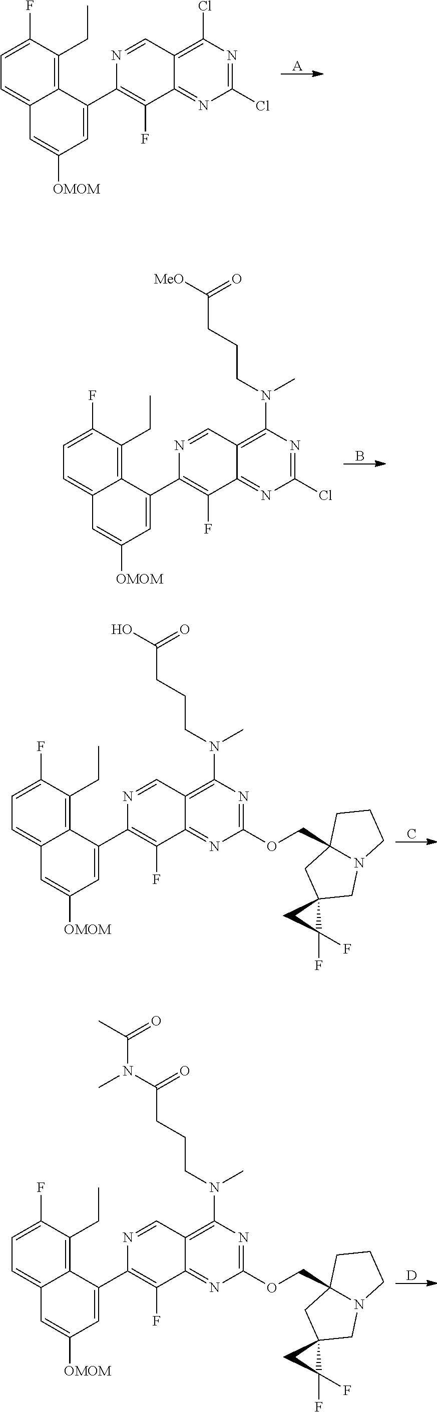

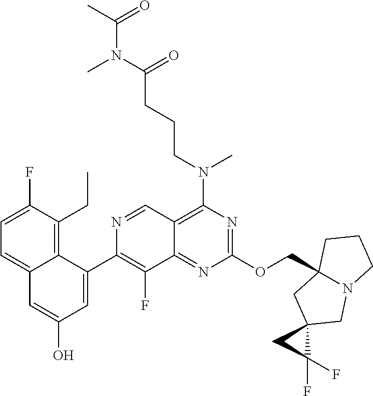

Step A. methyl 4-((2-chloro-7-(8-ethyl-7-fluoro-3-(methoxymethoxy)naphthalen-1-yl)-8-fluoropyrido[4,3-d]pyrimidin-4-yl)(methyl)amino)butanoate: To a solution of 2,4-dichloro-7-(8-ethyl-7-fluoro-3-(methoxymethoxy)naphthalen-1-yl)-8-fluoropyrido[4,3-d]pyrimidine (1.16 g, 1.0 eq.) in DCM (15 mL) were added DIEA (3.33 g, 10 eq.) and methyl 4-(methylamino)butanoate (432 mg, 1.0 eq.) at −40 °C. The reaction was stirred at −40 °C for 1 hour. The mixture was filtered, diluted with water (20 mL) and extracted with ethyl acetate (3×20 mL). The combined organic layers were washed with brine (30 mL), dried over anhydrous sodium sulfate, concentrated, and purified with column chromatography [SiO₂, petroleum ether:ethyl acetate=20:1 to 3:1] to afford the title compound (830 mg, 32% yield) as yellow solid; LCMS (ESI, M+1): m/z=545.3.

Step B. 4-((2-(((1S,7a'S)-2,2-difluorodihydro-1′H,3′H-spiro[cyclopropane-1,2′-pyrrolizin]-7a′ (5′H)-yl)methoxy)-7-(8-ethyl-7-fluoro-3-(methoxymethoxy)naphthalen-1-yl)-8-fluoropyrido[4,3-d]pyrimidin-4-yl)(methyl)amino)butanoic acid: To a solution of ((1S,7a'S)-2,2-difluorodihydro-1′H,3′H-spiro[cyclopropane-1,2′-pyrrolizin]-7a′ (5′H)-yl)methanol (248 mg, 0.9 eq.) in THF (5 mL) was added sodium hydride (163 mg, 3.0 equiv, 60% purity) at 0 °C under N₂ atmosphere. The reaction was stirred at 0 °C for 0.5 hour and then a solution of methyl 4-((2-chloro-7-(8-ethyl-7-fluoro-3-(methoxymethoxy)naphthalen-1-yl)-8-fluoropyrido[4,3-d]pyrimidin-4-yl)(methyl)amino)butanoate (740 mg, 1.0 eq.) in THF (5 mL) was added at 0 °C. The reaction was stirred at 25 °C for 12 hours under nitrogen atmosphere. The mixture was quenched with water (20 mL) at 0 °C and extracted with ethyl acetate (3×20 mL). The combined organic layers were washed with brine (30 mL), dried over anhydrous sodium sulfate, concentrated and purified with prep-HPLC [column: Phenomenx luna C18 150×40 mm×15 μm; mobile phase: water (FA)-ACN; gradient: 25%-55% B over 15 min] to afford the title compound (40.0 mg, 3.5% yield) as light yellow solid; LCMS (ESI, M+1): m/z=698.3.

Step C. N-acetyl-4-((2-(((1S,7a'S)-2,2-difluorodihydro-1′H,3′H-spiro[cyclopropane-1,2′-pyrrolizin]-7a′ (5′H)-yl)methoxy)-7-(8-ethyl-7-fluoro-3-(methoxymethoxy)naphthalen-1-yl)-8-fluoropyrido[4,3-d]pyrimidin-4-yl)(methyl)amino)-N-methylbutanamide: To a solution of 4-((2-(((1S,7a'S)-2,2-difluorodihydro-1′H,3′H-spiro[cyclopropane-1,2′-pyrrolizin]-7a′ (5′H)-yl)methoxy)-7-(8-ethyl-7-fluoro-3-(methoxymethoxy)naphthalen-1-yl)-8-fluoropyrido[4,3-d]pyrimidin-4-yl)(methyl)amino)butanoic acid (40.0 mg, 1.0 eq.) in 2-methyltetrahydrofuran (0.5 mL) were added DIEA (30.6 mg, 5.0 eq.), N-methylacetamide (17.3 mg, 5.0 eq.) and CMPI (60.5 mg, 5.0 eq.). The reaction was stirred at 80 °C for 3 hours. The mixture was quenched with water (5 mL) and extracted with ethyl acetate (3×5 mL). The combined organic layers were washed with brine (2×5 mL), dried over anhydrous sodium sulfate, concentrated and purified with prep-HPLC [column: Phenomenx luna C18 150×25 mm×10 μm; mobile phase: water (FA)-ACN; gradient: 22%-52% B over 9 min] to afford the title compound (20.0 mg, 56% yield) as orange solid; LCMS (ESI, M+1): m/z=753.4.

Step D. N-acetyl-4-((2-(((1S,7a'S)-2,2-difluorodihydro-1′H,3′H-spiro[cyclopropane-1,2′-pyrrolizin]-7a′ (5′H)-yl)methoxy)-7-(8-ethyl-7-fluoro-3-hydroxynaphthalen-1-yl)-8-fluoropyrido[4,3-d]pyrimidin-4-yl)(methyl)amino)-N-methylbutanamide: To a solution of N-acetyl-4-((2-(((1S,7a'S)-2,2-difluorodihydro-1′H,3′H-spiro[cyclopropane-1,2′-pyrrolizin]-7a′ (5′H)-yl)methoxy)-7-(8-ethyl-7-fluoro-3-(methoxymethoxy)naphthalen-1-yl)-8-fluoropyrido[4,3-d]pyrimidin-4-yl)(methyl)amino)-N-methylbutanamide (20 mg, 1.0 eq.) in DCM (0.3 mL) was added TFA (0.1 mL). The reaction was stirred at 25 °C for 1 hour. The mixture was concentrated, diluted with ethyl acetate (5 mL) and neutralized with DIEA at −40 °C. The mixture was diluted with water (5 mL) and extracted with ethyl acetate (3×5 mL). The combined organic layers were washed with brine (5 mL), dried over anhydrous sodium sulfate, concentrated and purified with prep-HPLC [column: Phenomenx luna C18 250×50 mm×15 μm; mobile phase: water (FA)-ACN; gradient: 20%-50% B over 9 min] to afford the title compound (3.91 mg, 20% yield, 0.22HCOOH) as off-white solid; ¹H NMR (400 MHz, DMSO-d₆) δ=9.97-9.91 (m, 1H), 9.28 (s, 1H), 7.76 (dd, J=6.0, 8.8 Hz, 1H), 7.42-7.27 (m, 2H), 7.02 (d, J=2.8 Hz, 1H), 4.31-4.15 (m, 2H), 3.99-3.79 (m, 2H), 3.54 (s, 3H), 3.13-3.06 (m, 4H), 3.04-2.95 (m, 1H), 2.85 (t, J=6.8 Hz, 2H), 2.76-2.65 (m, 1H), 2.53 (br s, 1H), 2.40-2.25 (m, 5H), 2.16-1.95 (m, 5H), 1.83-1.70 (m, 2H), 1.62-1.42 (m, 3H), 0.73 (t, J=7.2 Hz, 3H); LCMS (ESI, M+1): m/z=709.5.

### Example 294

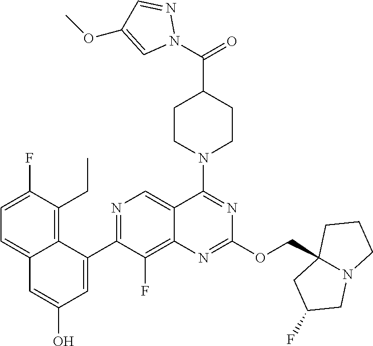 [SMILES: CCc1c(F)ccc2cc(O)cc(-c3ncc4c(N5CCC(C(=O)n6cc(OC)cn6)CC5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(4-methoxy-1H-pyrazol-1-yl)methanone

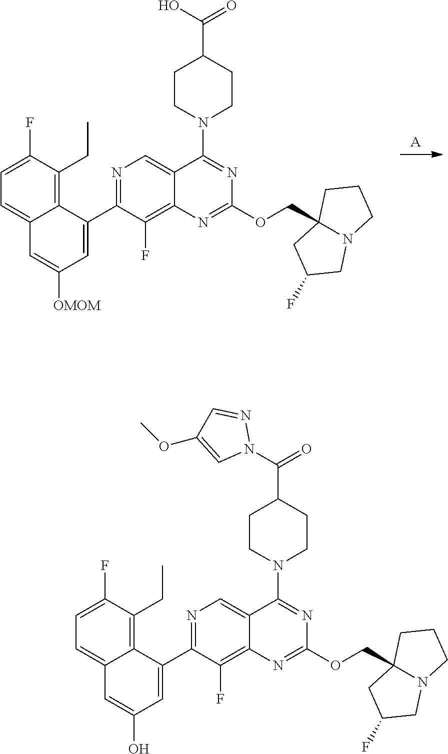

Step A. (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidin-4-yl)(4-methoxy-1H-pyrazol-1-yl)methanone: To a solution of 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)piperidine-4-carboxylic acid (300 mg, 1.0 eq.) and 4-methoxy-1H-pyrazole (47.3 mg, 3.0 eq.) in DMF (2 mL) were added HATU (122 mg, 2.0 eq.) and DIEA (104 mg, 5.0 eq.). The reaction was stirred at 25 °C for 12 hours. The mixture was filtered, and the filtrate was purified with prep-HPLC [Waters Xbridge Prep OBD C18 150×40 mm×10 μm; A: water (NH₄HCO₃), B: ACN, B %: 45%-75% over 20 min]. The desired fractions were combined, diluted with water (50 mL) and extracted with dichloromethane (2×30 mL). The organic layers were dried over anhydrous sodium sulfate and concentrated to afford the title compound (12.3 mg, 10% yield) as yellow solid; ¹H NMR (400 MHz, CHLOROFORM-d₃) δ=8.87 (d, J=2.0 Hz, 1H), 7.70 (s, 1H), 7.51-7.35 (m, 2H), 7.14-7.04 (m, 2H), 7.00-6.82 (m, 1H), 5.39-5.07 (m, 1H), 4.54 (br d, J=12.8 Hz, 2H), 4.28-4.12 (m, 2H), 4.01-3.86 (m, 1H), 3.73 (s, 3H), 3.50-3.32 (m, 2H), 3.28-3.01 (m, 3H), 2.98-2.83 (m, 1H), 2.48-2.32 (m, 1H), 2.30-2.13 (m, 2H), 2.12-1.92 (m, 6H), 1.90-1.76 (m, 3H), 0.73 (br t, J=7.2 Hz, 3H); LCMS (ESI, M+1): m/z=702.3.

### Example 295

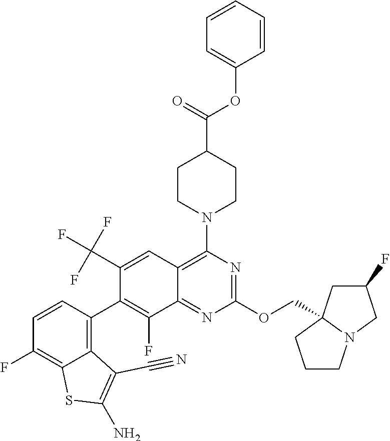 [SMILES: N#Cc1c(N)sc2c(F)ccc(-c3c(C(F)(F)F)cc4c(N5CCC(C(=O)Oc6ccccc6)CC5)nc(OC[C@@]56CCCN5C[C@H](F)C6)nc4c3F)c12]

### phenyl 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylate

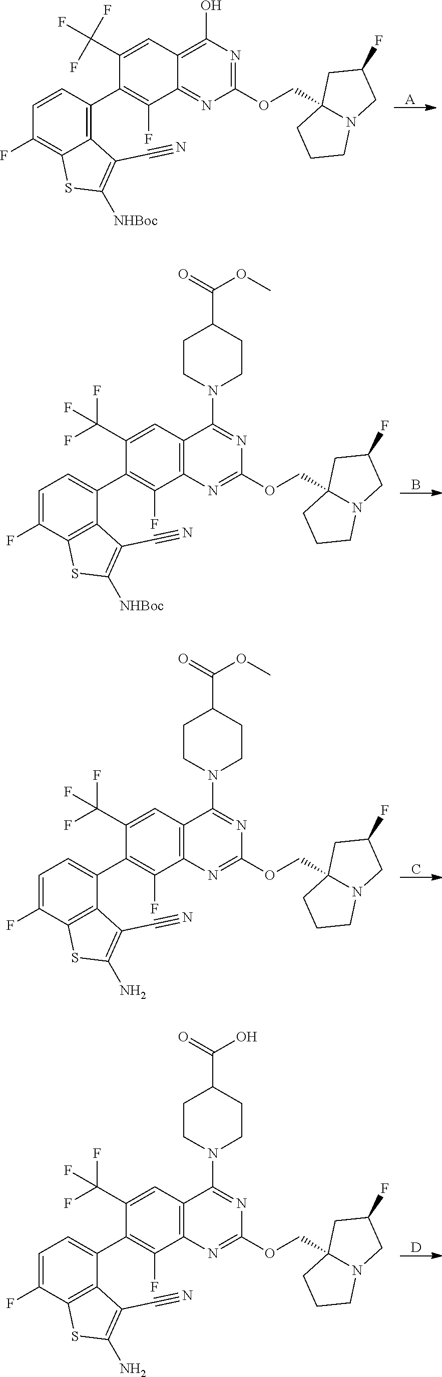

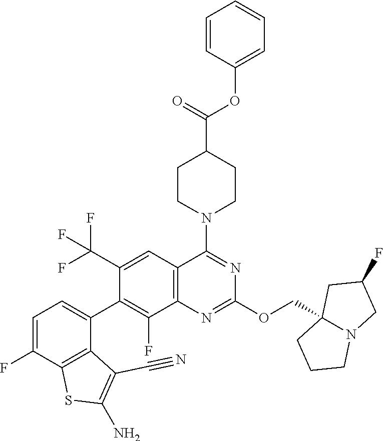 [SMILES: N#Cc1c(N)sc2c(F)ccc(-c3c(C(F)(F)F)cc4c(N5CCC(C(=O)Oc6ccccc6)CC5)nc(OC[C@@]56CCCN5C[C@H](F)C6)nc4c3F)c12]

Step A. methyl 1-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylate: To a mixture of tert-butyl (3-cyano-7-fluoro-4-(8-fluoro-2-(((2R,7aS)-2-fluorotetrahydro-1H-pyrrolizin-7a(5H)-yl)methoxy)-4-hydroxy-6-(trifluoromethyl)quinazolin-7-yl)benzo[b]thiophen-2-yl)carbamate (400 mg, 1.0 eq.) and methyl piperidine-4-carboxylate (211 mg, 2.5 eq.) in DMSO (3.0 mL) were added PyBOP (613 mg, 2.0 eq.) and TEA (179 mg, 3.0 eq.). The reaction was stirred at 20 °C for 14 hours. The mixture was diluted with DMSO (0.5 mL) and purified with reversed phase flash chromatography [water (0.1% formic acid)/acetonitrile=1/1] to afford the title compound (407 mg, 84% yield) as yellow solid; LCMS (ESI, M+1): m/z=805.4.

Step B. methyl 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylate: To a solution of methyl 1-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylate (400 mg, 1.0 eq.) in DCM (3.0 mL) was added TFA (3.0 mL) at 0 °C. The reaction was stirred at 20 °C for 1 hour. The mixture was concentrated. The residue was partitioned bewteen EtOAc (20 mL) and saturated NaHCO₃ aqueous solution (20 mL) at 0 °C. The aqueous solution was extracted with EtOAc (4×10 mL). The combined organic layers were washed brine (15 mL), dried over anhydrous sodium sulfate, and concentrated to afford the title compound (350 mg, crude) as pink solid; LCMS (ESI, M+1): m/z=705.3.

Step C. 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylic acid: To a solution of methyl 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylate (350 mg, 1.0 eq.) in MeOH (3.0 mL) was added LiOH·H₂O (1.5 M in H₂O, 993 μL, 3.0 eq.). The reaction was stirred at 20 °C for 1 hour. The mixture was neutralized with 2M HCl at 0 °C, concentrated and purified with reversed phase flash chromatography [water (0.1% formic acid)/acetonitrile=2/1] to afford the title compound (205 mg, 57% yield) as a light yellow solid; LCMS (ESI, M+1): m/z=691.3.

Step D. phenyl 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylate: To a solution of 1-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)piperidine-4-carboxylic acid (90.0 mg, 1.0 eq.) and phenol (36.8 mg, 3.0 eq.) in DMF (0.9 mL) were added EDCI (45.0 mg, 1.8 eq.), DIEA (50.5 mg, 3.0 eq.) and HOBt (33.5 mg, 1.9 eq.). The reaction was stirred at 20 °C for 14 hours. The mixture was purified with reversed phase flash chromatography [water (0.1% formic acid)/acetonitrile=2/3] and prep-HPLC [column: Welch Xtimate C18 150×25 mm×5 μm; mobile phase: (water (0.1% formic acid)/acetonitrile; gradient: 26%-56% B over 10 minutes] to afford the title compound (8.73 mg, 8.6% yield) as a white solid; ¹H NMR (400 MHz, CHLOROFORM-d) δ=8.03 (s, 1H), 7.45-7.35 (m, 2H), 7.26-7.18 (m, 2H), 7.11 (dd, J=1.2, 8.6 Hz, 2H), 7.05-6.96 (m, 1H), 5.47-5.42 (m, 2H), 5.29 (d, J=53.2 Hz, 1H), 4.45-4.36 (m, 2H), 4.36-4.29 (m, 1H), 4.28-4.20 (m, 1H), 3.53-3.40 (m, 2H), 3.38-3.15 (m, 3H), 3.06-2.91 (m, 2H), 2.34-2.08 (m, 7H), 2.02-1.92 (m, 3H); LCMS (ESI, M+1): m/z=767.3.

### Example 296

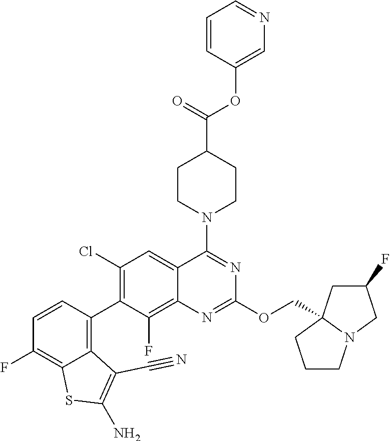 [SMILES: N#Cc1c(N)sc2c(F)ccc(-c3c(Cl)cc4c(N5CCC(C(=O)Oc6cccnc6)CC5)nc(OC[C@@]56CCCN5C[C@H](F)C6)nc4c3F)c12]

### pyridin-3-yl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

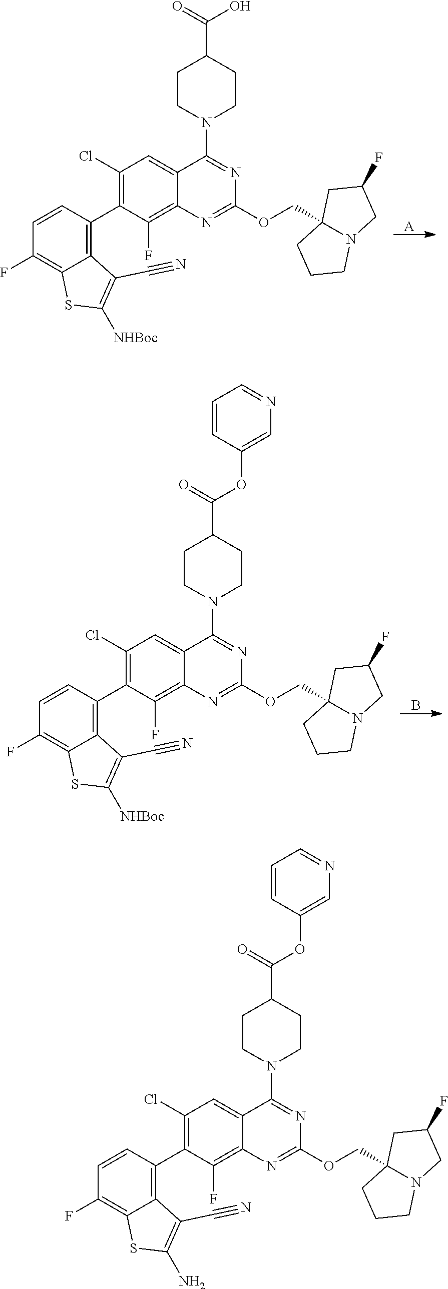

Step A. pyridin-3-yl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylic acid (200 mg, 1.0 eq.) and pyridin-3-ol (75.0 mg, 3.0 eq.) in DMF (1 mL) were added EDCI (76.0 mg, 1.5 eq.) and DMAP (48.0 mg, 1.5 eq.). The reaction mixture was stirred at 60 °C for 1 hour. The mixture was purified with prep-HPLC to afford the title compound (125 mg, 46% yield) as yellow solid; ¹H NMR (400 MHz, DMSO-d₆) δ=8.59-8.42 (m, 2H), 7.90 (s, 1H), 7.68 (ddd, J=1.2, 2.8, 8.4 Hz, 1H), 7.51 (dd, J=4.8, 8.4 Hz, 1H), 7.35-7.15 (m, 2H), 5.53-5.27 (m, 1H), 4.44-4.19 (m, 4H), 3.49 (d, J=12.0 Hz, 4H), 3.20-3.01 (m, 3H), 2.35-2.10 (m, 5H), 2.05-1.83 (m, 5H), 1.47 (s, 9H); LCMS (ESI, M+1): m/z=834.3.

Step B. pyridin-3-yl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate: To a solution of pyridin-3-yl 1-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate (125.0 mg, 1.0 eq.) in DCM (1 mL) was added TFA (1 mL). The reaction was stirred at 15 °C for 0.5 hour. The mixture was concentrated, diluted with ethyl acetate (15 mL) and washed with saturated NaHCO₃ (3×15 mL). The organic layer was dried over anhydrous sodium sulfate, concentrated, and purified with prep-HPLC to afford the title compound (34.64 mg, 31% yield) as white solid; ¹H NMR (400 MHz, DMSO-d₆) δ=8.54-8.43 (m, 2H), 8.14 (s, 2H), 7.87 (s, 1H), 7.69 (ddd, J=1.2, 2.8, 8.4 Hz, 1H), 7.50 (dd, J=4.8, 8.4 Hz, 1H), 7.27 (dd, J=5.2, 8.4 Hz, 1H), 7.21-7.09 (m, 1H), 5.40-5.14 (m, 1H), 4.29 (t, J=14.4 Hz, 2H), 4.15-4.07 (m, 1H), 4.01 (dd, J=2.8, 10.4 Hz, 1H), 3.52-3.38 (m, 2H), 3.18-2.98 (m, 4H), 2.88-2.77 (m, 1H), 2.28-2.10 (m, 3H), 2.09-1.91 (m, 4H), 1.89-1.73 (m, 3H); LCMS (ESI, M+1): m/z=734.2.

### Example 297

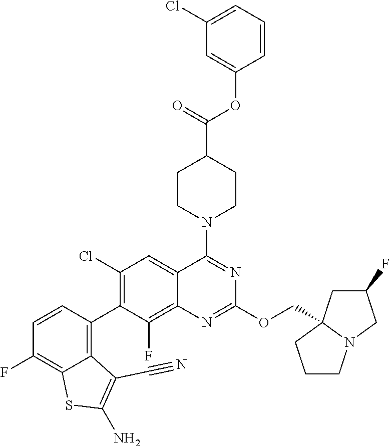 [SMILES: N#Cc1c(N)sc2c(F)ccc(-c3c(Cl)cc4c(N5CCC(C(=O)Oc6cccc(Cl)c6)CC5)nc(OC[C@@]56CCCN5C[C@H](F)C6)nc4c3F)c12]

### 3-chlorophenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 296. The title compound was obtained as white solid; ¹H NMR (400 MHz, METHANOL-d₄) δ=7.88 (d, J=1.2 Hz, 1H), 7.43-7.36 (m, 1H), 7.31-7.26 (m, 1H), 7.25-7.18 (m, 2H), 7.13-6.97 (m, 2H), 5.48-5.23 (m, 1H), 4.52-4.42 (m, 2H), 4.41-4.25 (m, 2H), 3.62-3.47 (m, 2H), 3.46-3.33 (m, 3H), 3.15-3.02 (m, 2H), 2.48-2.15 (m, 5H), 2.14-1.80 (m, 5H); LCMS (ESI, M+1): m/z=767.2.

### Example 298

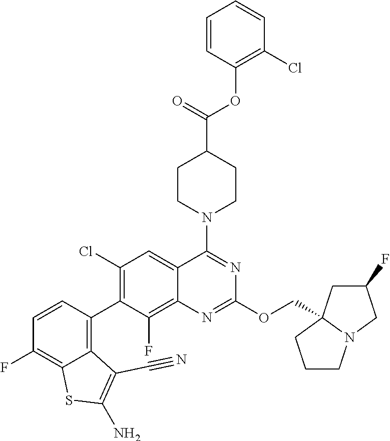 [SMILES: N#Cc1c(N)sc2c(F)ccc(-c3c(Cl)cc4c(N5CCC(C(=O)Oc6ccccc6Cl)CC5)nc(OC[C@@]56CCCN5C[C@H](F)C6)nc4c3F)c12]

### 2-chlorophenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 296. The title compound was obtained as white solid; ¹H NMR (400 MHz, METHANOL-d₄) δ=7.88 (d, J=1.2 Hz, 1H), 7.50 (dd, J=1.2, 8.0 Hz, 1H), 7.27 (dt, J=1.6, 7.6 Hz, 2H), 7.24-7.16 (m, 2H), 7.04 (t, J=8.8 Hz, 1H), 5.46-5.22 (m, 1H), 4.52-4.41 (m, 2H), 4.40-4.23 (m, 2H), 3.65-3.50 (m, 2H), 3.46-3.32 (m, 2H), 3.27 (s, 1H), 3.19-3.03 (m, 2H), 2.46-2.23 (m, 4H), 2.22-1.89 (m, 6H); LCMS (ESI, M+1): m/z=767.1.

### Example 299

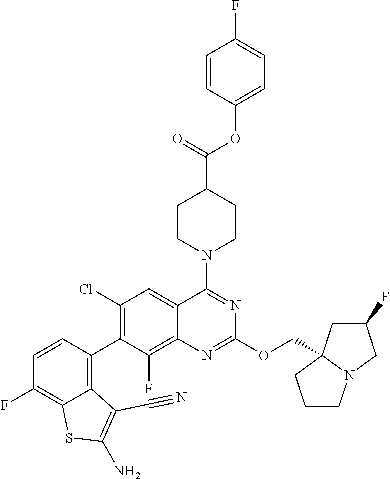 [SMILES: N#Cc1c(N)sc2c(F)ccc(-c3c(Cl)cc4c(N5CCC(C(=O)Oc6ccc(F)cc6)CC5)nc(OC[C@@]56CCCN5C[C@H](F)C6)nc4c3F)c12]

### 4-fluorophenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 296. The title compound was obtained as white solid; ¹H NMR (400 MHz, METHANOL-d₄) δ=7.88 (s, 1H), 7.21 (dd, J=5.2, 8.4 Hz, 1H), 7.14 (d, J=6.4 Hz, 4H), 7.04 (t, J=8.8 Hz, 1H), 5.46-5.22 (m, 1H), 4.53-4.41 (m, 2H), 4.40-4.24 (m, 2H), 3.63-3.46 (m, 2H), 3.46-3.33 (m, 2H), 3.26 (d, J=5.2 Hz, 1H), 3.13-3.00 (m, 2H), 2.47-2.16 (m, 5H), 2.13-1.90 (m, 5H); LCMS (ESI, M+1): m/z=751.2.

### Example 300

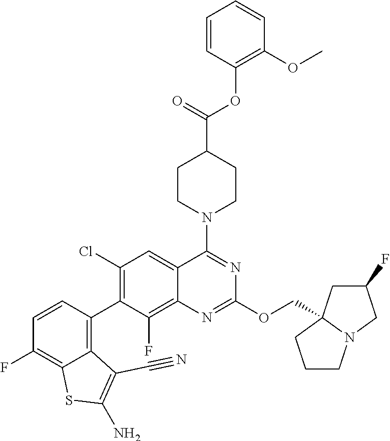 [SMILES: COc1ccccc1OC(=O)C1CCN(c2nc(OC[C@@]34CCCN3C[C@H](F)C4)nc3c(F)c(-c4ccc(F)c5sc(N)c(C#N)c45)c(Cl)cc23)CC1]

### 2-methoxyphenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 296. The title compound was obtained as white solid; ¹H NMR (400 MHz, METHANOL-d₄) δ=7.88 (s, 1H), 7.28-7.19 (m, 2H), 7.12-7.04 (m, 2H), 7.04-6.92 (m, 2H), 5.44-5.23 (m, 1H), 4.48-4.39 (m, 2H), 4.39-4.26 (m, 2H), 3.82 (s, 3H), 3.69-3.53 (m, 2H), 3.47-3.35 (m, 2H), 3.27 (m, 1H), 3.16-3.03 (m, 2H), 2.45-2.17 (m, 5H), 2.14-1.88 (m, 5H); LCMS (ESI, M+1): m/z=763.3.

### Example 301

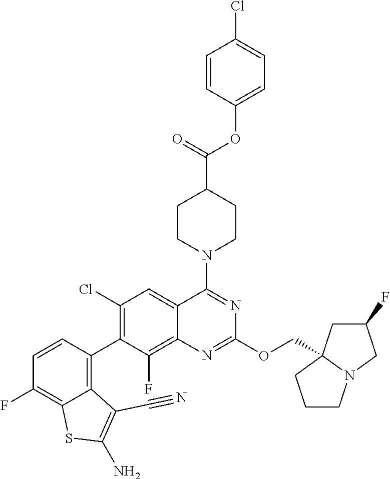 [SMILES: N#Cc1c(N)sc2c(F)ccc(-c3c(Cl)cc4c(N5CCC(C(=O)Oc6ccc(Cl)cc6)CC5)nc(OC[C@@]56CCCN5C[C@H](F)C6)nc4c3F)c12]

### 4-chlorophenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 296. The title compound was obtained as white solid; ¹H NMR (400 MHz, METHANOL-d4) δ=7.89 (d, J=1.2 Hz, 1H), 7.47-7.36 (m, 2H), 7.21 (dd, J=5.2, 8.4 Hz, 1H), 7.17-7.10 (m, 2H), 7.09-6.98 (m, 1H), 5.47-5.25 (m, 1H), 4.46 (dt, J=4.4, 8.9 Hz, 2H), 4.42-4.29 (m, 2H), 3.60-3.43 (m, 3H), 3.43-3.36 (m, 2H), 3.16-3.03 (m, 2H), 2.47-2.19 (m, 5H), 2.12-1.93 (m, 5H); LCMS (ESI, M+1): m/z=767.1.

### Example 302

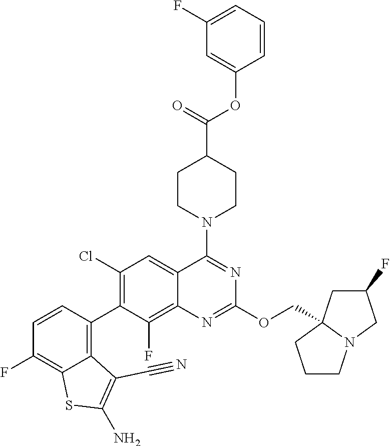 [SMILES: N#Cc1c(N)sc2c(F)ccc(-c3c(Cl)cc4c(N5CCC(C(=O)Oc6cccc(F)c6)CC5)nc(OC[C@@]56CCCN5C[C@H](F)C6)nc4c3F)c12]

### 3-fluorophenyl 1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)piperidine-4-carboxylate

Synthesized according to the method used for Example 296. The title compound was obtained as white solid; ¹H NMR (400 MHz, METHANOL-d₄) δ=7.88 (s, 1H), 7.42 (q, J=7.6 Hz, 1H), 7.21 (dd, J=5.2, 8.4 Hz, 1H), 7.07-6.95 (m, 4H), 5.43-5.23 (m, 1H), 4.56-4.41 (m, 2H), 4.38-4.21 (m, 2H), 3.59-3.47 (m, 2H), 3.44-3.35 (m, 1H), 3.27 (d, J=4.8 Hz, 2H), 3.11-3.02 (m, 2H), 2.42-2.15 (m, 5H), 2.11-1.90 (m, 5H); LCMS (ESI, M+1): m/z=751.2.

### Example 303

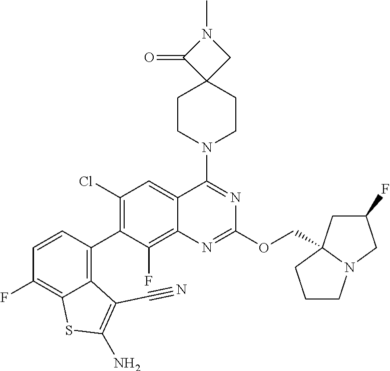 [SMILES: CN1CC2(CCN(c3nc(OC[C@@]45CCCN4C[C@H](F)C5)nc4c(F)c(-c5ccc(F)c6sc(N)c(C#N)c56)c(Cl)cc34)CC2)C1=O]

### 2-amino-4-(6-chloro-8-fluoro-2-(((2R,7aS)-2-fluorotetrahydro-1H-pyrrolizin-7a(5H)-yl)methoxy)-4-(2-methyl-1-oxo-2,7-diazaspiro[3.5]nonan-7-yl)quinazolin-7-yl)-7-fluorobenzo[b]thiophene-3-carbonitrile

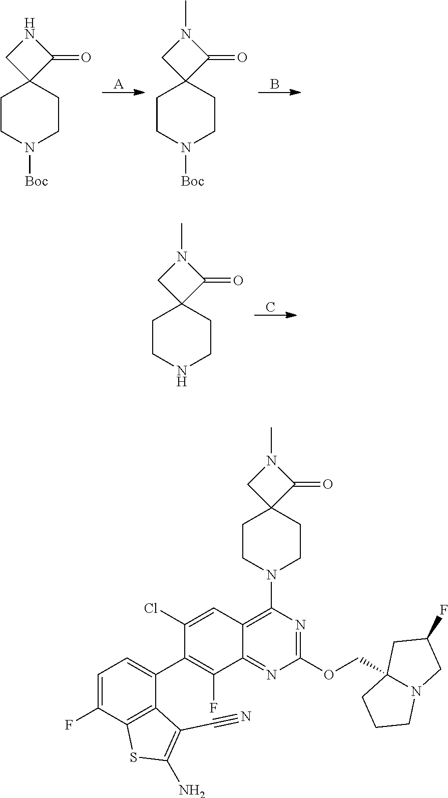

Step A. tert-butyl 2-methyl-1-oxo-2,7-diazaspiro[3.5]nonane-7-carboxylate; To a solution of tert-butyl 1-oxo-2,7-diazaspiro[3.5]nonane-7-carboxylate (1.00 g, 1.0 eq.) in THE (10 mL) were added NaH (333 mg, 2.0 eq.) at 0 °C. The reaction was stirred at 0 °C for 1 hour. A solution of MeI (886 mg, 1.5 eq.) in THF (2 mL) was added dropwise at 0 °C. The reaction was stirred at 0 °C for 1 hour. The mixture was quenched with saturated NH₄Cl (20 mL) at 0 °C and extracted with ethyl acetate (3×50 mL). The combined organic layers were washed with brine (3×50 mL), dried over anhydrous Na₂SO₄, filtered, concentrated, and purified with column chromatography [SiO₂, DCM/MeOH=50/1 to 10/1] to afford title compound (800 mg, 75% yield) as yellow solid; ¹H NMR (400 MHz, CHLOROFORM-d) δ=3.76-3.70 (m, 2H), 3.36-3.28 (m, 2H), 3.09 (s, 2H), 2.83 (s, 3H), 1.94-1.89 (m, 2H), 1.73-1.67 (m, 2H), 1.45 (s, 9H); LCMS (ESI, M+23): m/z=277.0

Step B. 2-methyl-2,7-diazaspiro[3.5]nonan-1-one: To a solution of tert-butyl 2-methyl-1-oxo-2,7-diazaspiro[3.5]nonane-7-carboxylate (800 mg, 1.0 eq.) in DCM (9 mL) was added TFA (4.61 g, 13 eq.) at 0 °C. The reaction was stirred at 0 °C for 0.5 hour. The mixture was concentrated to afford title compound (195 mg, crude) as yellow oil; LCMS (ESI, M+1): m/z=155.2

Step C. 2-amino-4-(6-chloro-8-fluoro-2-(((2R,7aS)-2-fluorotetrahydro-1H-pyrrolizin-7a(5H)-yl)methoxy)-4-(2-methyl-1-oxo-2,7-diazaspiro[3.5]nonan-7-yl)quinazolin-7-yl)-7-fluorobenzo[b]thiophene-3-carbonitrile: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (50.0 mg, 1.0 eq.) in DMF (1 mL) were added 2-methyl-2,7-diazaspiro[3.5]nonan-1-one (141 mg, 10 eq.), PyBOP (71.5 mg, 1.5 eq.) and DIEA (35.5 mg, 3.0 eq.) at 25 °C. The reaction was stirred at 25 °C for 1 hour. The mixture was filtered and the filtrate was purified with prep-HPLC [column: Phenomenex luna C18 150×25 mm×10 μm; A: water (0.1% FA), B: ACN, B %: 22%-52% over 11 min] to afford title compound (2.63 mg, 4% yield, 0.22HCOOH); ¹H NMR (400 MHz, METHANOL-d₄) δ=7.88 (d, J=1.6 Hz, 1H), 7.22 (dd, J=5.2, 8.4 Hz, 1H), 7.10-7.00 (m, 1H), 5.48-5.25 (m, 1H), 4.41-4.26 (m, 2H), 4.25-4.16 (m, 2H), 3.88-3.77 (m, 2H), 3.52-3.35 (m, 3H), 3.32-3.26 (m, 2H), 3.13-3.04 (m, 1H), 2.89 (s, 3H), 2.46-2.25 (m, 2H), 2.24-2.13 (m, 3H), 2.09-2.01 (m, 4H), 1.98-1.88 (m, 1H); LCMS (ESI, M+1): m/z=682.1

### Example 304

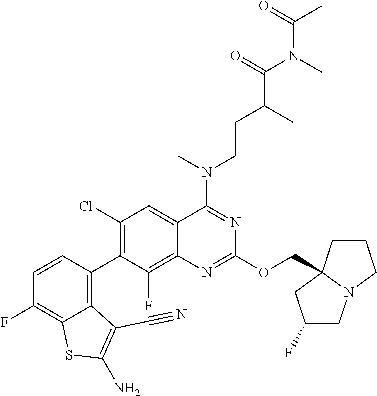 [SMILES: CC(=O)N(C)C(=O)C(C)CCN(C)c1nc(OC[C@@]23CCCN2C[C@H](F)C3)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N,2-dimethylbutanamide

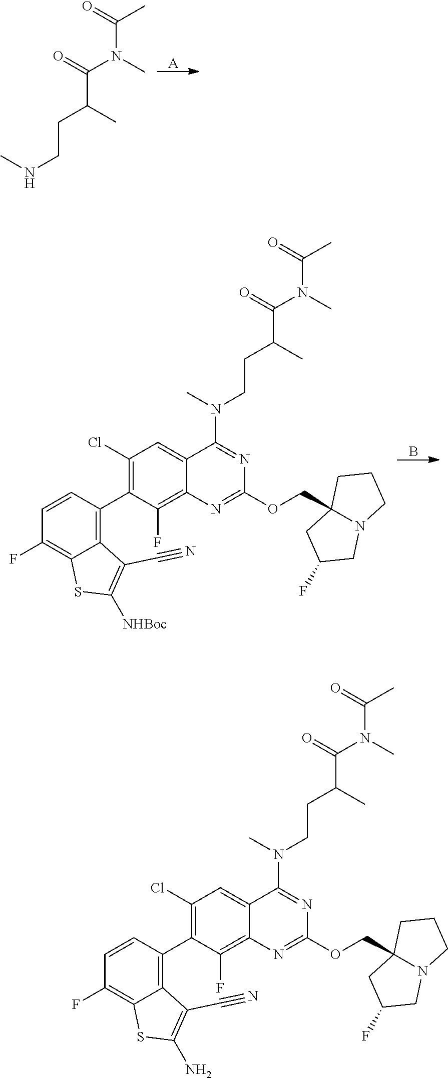

Step A. tert-butyl (4-(6-chloro-8-fluoro-2-(((2R,7aS)-2-fluorotetrahydro-1H-pyrrolizin-7a(5H)-yl)methoxy)-4-(methyl (3-methyl-4-(N-methylacetamido)-4-oxobutyl)amino)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (120 mg, 1.0 eq.) in DMF (2 mL) were added PyBOP (145 mg, 1.5 eq.) and DIEA (120 mg, 5.0 eq.). The reaction was stirred at 25 °C for 0.5 hour and then N-acetyl-N,2-dimethyl-4-(methylamino)butanamide (86.5 mg, 2.5 eq.) was added. The reaction was stirred at 25 °C for 1 hour. The mixture was filtered, concentrated and purified with prep-HPLC [column: Waters Xbridge Prep OBD C18 150×40 mm×10 μm; mobile phase: water (NH₄HCO₃)-ACN; gradient: 58%-88% B over 15 min] to afford the title compound (40.0 mg, 26% yield) as yellow oil; LCMS (ESI, M+1): m/z=814.2.

Step B. N-acetyl-4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N,2-dimethylbutanamide: To a solution of tert-butyl (4-(6-chloro-8-fluoro-2-(((2R,7aS)-2-fluorotetrahydro-1H-pyrrolizin-7a(5H)-yl)methoxy)-4-(methyl (3-methyl-4-(N-methylacetamido)-4-oxobutyl)amino)quinazolin-7-yl)-3-cyano-7-fluorobenzo[b]thiophen-2-yl)carbamate (40.0 mg, 1.0 eq.) in DCM (0.6 mL) was added TFA (0.2 mL). The reaction was stirred at 25 °C for 0.5 hour. The mixture was concentrated, diluted with ethyl acetate (5 mL), and neutralized with DIEA at −40 °C. The mixture was washed with water (5 mL), dried over anhydrous sodium sulfate, concentrated and purified with prep-HPLC [column: Waters Xbridge 150×25 mm×5 μm; mobile phase: water (NH₄HCO₃)-ACN; gradient: 48%-78% B over 10 min] to afford the title compound (18.3 mg, 51% yield) as white solid; ¹H NMR (400 MHz, METHANOL-d₄)_{8=8.06} (d, J=2.6 Hz, 1H), 7.23-7.16 (m, 1H), 7.07-7.00 (m, 1H), 5.41-5.22 (m, 1H), 4.31-4.16 (m, 2H), 4.09-3.92 (m, 1H), 3.87-3.68 (m, 1H), 3.57-3.43 (m, 4H), 3.28-3.13 (m, 3H), 3.12-3.04 (m, 3H), 3.04-2.97 (m, 1H), 2.41-2.25 (m, 5H), 2.23-2.10 (m, 2H), 2.02-1.83 (m, 4H), 1.24 (d, J=6.8 Hz, 3H); LCMS (ESI, M+1): m/z=714.3.

### Example 305

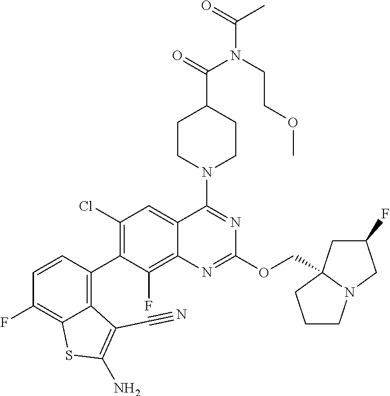 [SMILES: COCCN(C(C)=O)C(=O)C1CCN(c2nc(OC[C@@]34CCCN3C[C@H](F)C4)nc3c(F)c(-c4ccc(F)c5sc(N)c(C#N)c45)c(Cl)cc23)CC1]

### N-acetyl-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-(2-methoxyethyl)piperidine-4-carboxamide

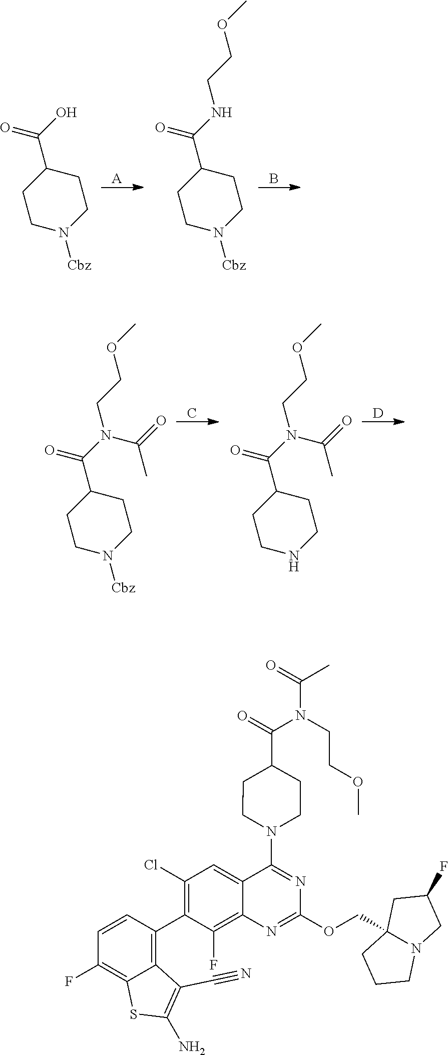

Step A. benzyl 4-((2-methoxyethyl)carbamoyl)piperidine-1-carboxylate: To a solution of 1-((benzyloxy)carbonyl)piperidine-4-carboxylic acid (2.00 g, 1.0 eq.) and 2-methoxyethanamine (628 mg, 1.1 eq.) in dimethylformamide (20 mL) were added diisopropylethylamine (3.93 g, 4.0 eq.) and HATU (4.91 g, 1.7 eq.) at 25 °C. The reaction was stirred at 25 °C for 4 hours. The mixture was concentrated under reduced pressure and purified with column chromatography [SiO₂, Petroleum ether/Ethyl acetate=5/1 to 0/1] to afford the title compound (750 mg, 31% yield) as yellow oil; LCMS (ESI, M+1): m/z=321.1; 1H NMR (400 MHz, CHLOROFORM-d) δ=7.39-7.30 (m, 5H), 5.90 (br s, 1H), 5.12 (s, 2H), 4.35-4.14 (m, 2H), 3.50-3.41 (m, 4H), 3.35 (s, 3H), 2.87-2.75 (m, 2H), 2.26 (tt, J=3.6, 7.2 Hz, 1H), 1.83 (br s, 2H), 1.73-1.61 (m, 2H)

Step B. benzyl 4-(acetyl(2-methoxyethyl)carbamoyl)piperidine-1-carboxylate: To a solution of benzyl 4-((2-methoxyethyl)carbamoyl)piperidine-1-carboxylate (600 mg, 1.0 eq.) in 2-methyltetrahydrofuran (6 mL) were added diisopropylethylamine (1.45 g, 6.0 eq.) and acetyl chloride (588 mg, 4.0 eq.) at 25 °C. The reaction was stirred at 80 °C for 2 hours. The mixture was diluted with dichloromethane (3 mL) and purified with column chromatography [SiO₂, Petroleum ether/Ethyl acetate=10/1 to 0/1] to afford the title compound (330 mg, 49% yield) as yellow oil; LCMS (ESI, M+1): m/z=363.1

Step C. N-acetyl-N-(2-methoxyethyl)piperidine-4-carboxamide: To a solution of benzyl 4-(acetyl(2-methoxyethyl)carbamoyl)piperidine-1-carboxylate (330 mg, 1.0 eq.) in ethyl acetate (10 mL) was added Pd/C (969 mg, 10% purity, 1.0 eq.) under nitrogen atmosphere at 25 °C. The reaction was degassed and purged with hydrogen 3 times, and the reaction was stirred at 25 °C for 0.5 hour under hydrogen (15 psi) atmosphere. The mixture was filtered, and the filter cake was washed with ethyl acetate (500 mL). The filtrate was concentrated under reduced pressure to afford the title compound (168 mg, 81% yield) as yellow oil; LCMS (ESI, M+1): m/z=229.1

Step D. N-acetyl-1-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N-(2-methoxyethyl)piperidine-4-carboxamide: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (80.0 mg, 1.0 eq.) and N-acetyl-N-(2-methoxyethyl)piperidine-4-carboxamide (167 mg, 5.0 eq.) in dimethylformamide (1 mL) were added diisopropylethylamine (56.8 mg, 3.0 eq.) and PyBOP (114 mg, 1.5 eq.). The reaction was stirred at 25 °C for 1 hour. The mixture was filtered and the filtrate was purified with prep-HPLC [Waters Xbridge 150×25 mm×5 μm; A: water (10 mM NH₄HCO₃), B: ACN, B %: 50%-80% over 10 min] to afford the title compound (10.6 mg, 9.0% yield) as pink solid; LCMS (ESI, M+1): m/z=756.3; ¹H NMR (400 MHz, DMSO-d₆) δ=8.12 (s, 2H), 7.83 (s, 1H), 7.27 (dd, J=5.2, 8.4 Hz, 1H), 7.20-7.09 (m, 1H), 5.40-5.15 (m, 1H), 4.39-4.24 (m, 2H), 4.14-4.06 (m, 1H), 4.00 (dd, J=2.8, 10.4 Hz, 1H), 3.87 (t, J=4.8 Hz, 2H), 3.55-3.35 (m, 5H), 3.24 (s, 3H), 3.08 (br d, J=7.6 Hz, 2H), 3.00 (s, 1H), 2.87-2.77 (m, 1H), 2.33 (s, 3H), 2.17-2.10 (m, 1H), 2.07-1.92 (m, 4H), 1.88-1.71 (m, 5H).

### Example 306

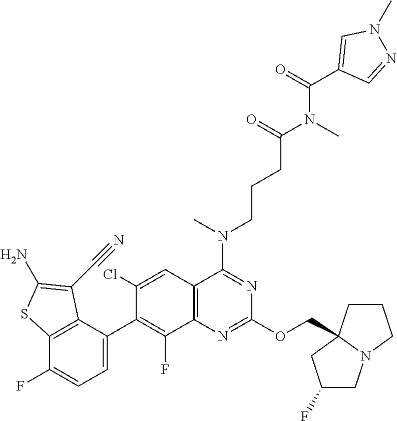 [SMILES: CN(C(=O)CCCN(C)c1nc(OC[C@@]23CCCN2C[C@H](F)C3)nc2c(F)c(-c3ccc(F)c4sc(N)c(C#N)c34)c(Cl)cc12)C(=O)c1cnn(C)c1]

### N-(4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoyl)-N, 1-dimethyl-1H-pyrazole-4-carboxamide

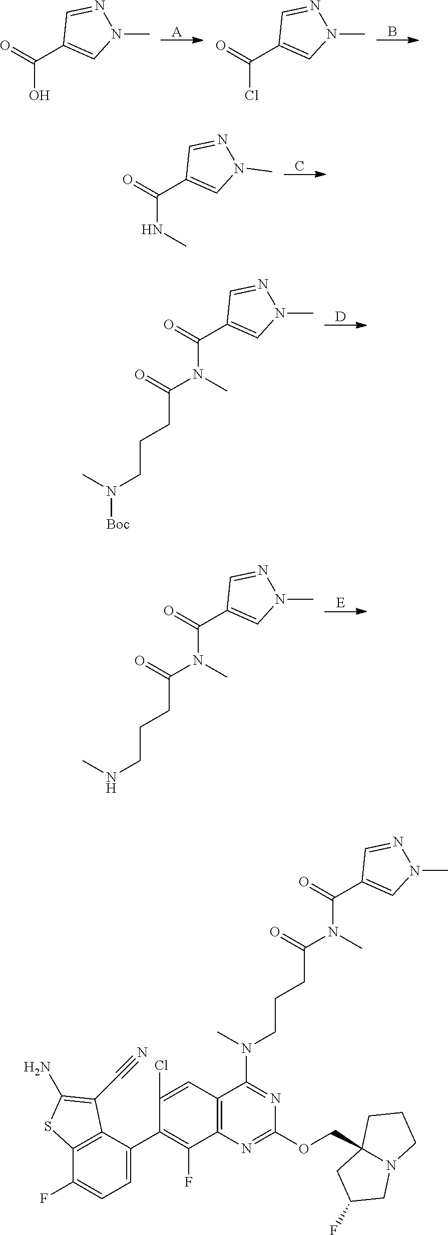

Step A. 1-methyl-1H-pyrazole-4-carbonyl chloride: To a solution of 1-methylpyrazole-4-carboxylic acid (2.50 g, 1.0 eq.) in DCM (30 mL) was added SOCl₂ (11.8 g, 7.2 mL, 5.0 eq.) at 0 °C. The reaction was stirred at 20 °C for 2 hours. The mixture was concentrated under vacuum to afford the title compound (2.00 g, crude) as yellow oil.

Step B. N,1-dimethyl-1H-pyrazole-4-carboxamide: To a solution of methanamine (2 M, 21 mL, 3.0 eq.) in THF (10 mL) was added 1-methyl-1H-pyrazole-4-carbonyl chloride (2.00 g, 1.0 eq.) at 25 °C. The reaction was stirred at 20 °C for 2 hours. The mixture was concentrated under reduced pressure and purified with reversed phase flash chromatography [C18, 0.1% formic acid condition] to afford the title compound (1.10 g, 41% yield) as yellow oil; LCMS (ESI, M+1): m/z=140.1

Step C. tert-butyl (4-(N,1-dimethyl-1H-pyrazole-4-carboxamido)-4-oxobutyl)(methyl)carbamate: To a solution of N, 1-dimethyl-1H-pyrazole-4-carboxamide (1.00 g, 1.0 eq.) and 4-((tert-butoxycarbonyl)(methyl)amino)butanoic acid (1.60 g, 1.1 eq.) in THF (30 mL) were added CMPI (3.41 g, 2.0 eq.) and DIEA (2.59 g, 3.5 mL, 3.0 eq.) at 25 °C. The reaction was stirred at 70 °C for 4 hours. The mixture was filtered and concentrated under reduced pressure to give a residue. The residue was purified with column chromatography [SiO₂, petroleum ether/ethyl acetate=1/0 to 2/1] to afford the title compound (763 mg, 29% yield) as yellow oil; LCMS (ESI, M+1, M+23): m/z=339.1, 361.3

Step D. N,1-dimethyl-N-(4-(methylamino)butanoyl)-1H-pyrazole-4-carboxamide: To a solution of tert-butyl (4-(N, 1-dimethyl-1H-pyrazole-4-carboxamido)-4-oxobutyl)(methyl)carbamate (400 mg, 1.0 eq.) in DCM (5 mL) was added TFA (2 mL) at 25 °C. The reaction was stirred at 25 °C for 1 hour. The mixture was concentrated under reduced pressure to afford the title compound (700 mg, crude, TFA salt) as yellow oil; LCMS (ESI, M+1): m/z=239.3.

Step E. N-(4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)butanoyl)-N, 1-dimethyl-1H-pyrazole-4-carboxamide: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (150 mg, 1.0 eq.) and N,1-dimethyl-N-(4-(methylamino)butanoyl)-1H-pyrazole-4-carboxamide (399 mg, 5.0 eq.) in DMF (5 mL) were added PyBOP (286 mg, 2.0 eq.) and DIEA (177 mg, 5.0 eq.) at 25 °C. The reaction was stirred at 25 °C for 1 hour. The mixture was diluted with water (20 mL) and filtered. The filter cake was purified with prep-HPLC [Waters Xbridge 150×25 mm×5 μm; A: water (10 mM NH₄HCO₃), B: ACN, B %: 40%-70% over 15 min] to afford the title compound (7.94 mg, 3.7% yield) as white solid; ¹H NMR (400 MHz, METHANOL-d₄) δ=8.10 (s, 1H), 7.92 (d, J=2.8 Hz, 1H), 7.82 (s, 1H), 7.21 (dd, J=5.2, 8.4 Hz, 1H), 7.03 (dd, J=8.4, 9.6 Hz, 1H), 5.38-5.17 (m, 1H), 4.25-4.03 (m, 3H), 3.91 (s, 3H), 3.83-3.71 (m, 1H), 3.52 (s, 3H), 3.26-3.08 (m, 6H), 3.04-2.88 (m, 2H), 2.86-2.73 (m, 1H), 2.37-2.03 (m, 5H), 1.98-1.78 (m, 3H); LCMS (ESI, M+1): m/z=766.3.

### Example 307

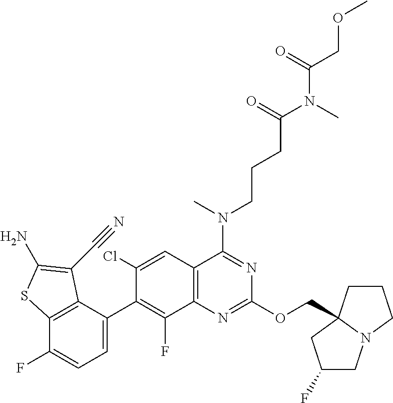 [SMILES: COCC(=O)N(C)C(=O)CCCN(C)c1nc(OC[C@@]23CCCN2C[C@H](F)C3)nc2c(F)c(-c3ccc(F)c4sc(N)c(C#N)c34)c(Cl)cc12]

### 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(2-methoxyacetyl)-N-methylbutanamide

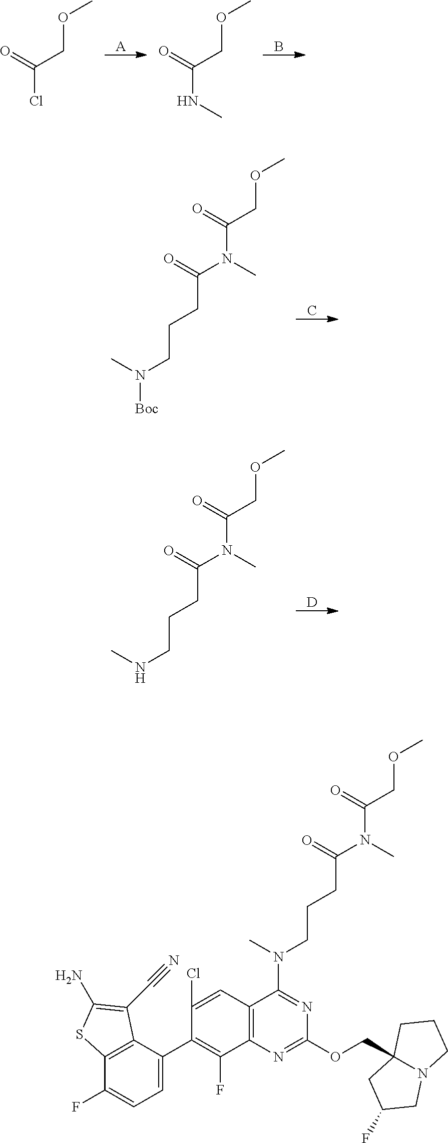

Step A. 2-methoxy-N-methylacetamide: To a solution of 2-methoxyacetyl chloride (5.00 g, 1.0 eq.) in THF (30 mL) was added MeNH₂ (3.10 g, 3.1 M, 2.2 eq.) at 0 °C. The reaction was stirred at 25 °C for 12 hours. The mixture was poured into water (100 mL) at 0 °C and extracted with ethyl acetate (3×100 mL). The combined organic layers were washed with brine (3×30 mL), dried over anhydrous Na₂SO₄, filtered and concentrated to afford title compound (4.75 g, crude) as yellow oil; ¹H NMR (400 MHz, DMSO-d₆) δ=7.72 (br s, 1H), 3.77 (s, 2H), 3.29 (s, 3H), 2.60 (d, J=4.8 Hz, 3H)

Step B. tert-butyl (4-(2-methoxy-N-methylacetamido)-4-oxobutyl)(methyl)carbamate: To a solution of 4-((tert-butoxycarbonyl)(methyl)amino)butanoic acid (4.00 g, 1.0 eq.) and 2-methoxy-N-methylacetamide (3.80 g, 2.0 eq.) in 2-Me THF (80 mL) were added CMPI (9.41 g, 2.0 eq.) and DIEA (7.14 g, 3.0 eq.) at 25 °C. The reaction was stirred at 80 °C for 0.5 hour. The mixture was quenched with water (30 mL) at 25 °C and extracted with ethyl acetate (3×50 mL). The combined organic layers were washed with brine (3×50 mL), dried over anhydrous Na₂SO₄, filtered, concentrated and purified with column chromatography [SiO₂, petroleum ether/ethyl acetate=5/1 to 1/1] to afford title compound (211 mg, 4% yield) as white oil; LCMS (ESI, M+23): 325.1.

Step C. N-(2-methoxyacetyl)-N-methyl-4-(methylamino)butanamide: To a solution of tert-butyl (4-(2-methoxy-N-methylacetamido)-4-oxobutyl)(methyl)carbamate (190 mg, 1.0 eq.) in DCM (2 mL) were added TFA (1.01 g, 14 eq.) at 0 °C. The reaction was stirred at 0 °C for 2 hours. The mixture was concentrated and adjusted to 7 with DIEA at −40 °C to afford title compound (110 mg, crude) as yellow oil.

Step D. 4-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)(methyl)amino)-N-(2-methoxyacetyl)-N-methylbutanamide: To a solution of 2-amino-4-(6-chloro-8-fluoro-2-(((2R,7aS)-2-fluorotetrahydro-1H-pyrrolizin-7a(5H)-yl)methoxy)-4-hydroxyquinazolin-7-yl)-7-fluorobenzo[b]thiophene-3-carbonitrile (55.0 mg, 1.0 eq.) in DMF (1.5 mL) were added N-(2-methoxyacetyl)-N-methyl-4-(methylamino)butanamide (81.5 mg, 4.0 eq.), PyBOP (71.5 mg, 1.5 eq.) and DIEA (35.5 mg, 3.0 eq.) at 25 °C. The reaction was stirred at 25 °C for 1 hour. The mixture was filtered and the filtrate was purified with prep-HPLC [column: Waters Xbridge 150×25 mm×5 μm; A: water (10 mM NH₄HCO₃), B: ACN, B %: 45%-75% over 9 min] to afford title compound (9.47 mg, 12% yield) as yellow solid; ¹H NMR (400 MHz, DMSO-d₆) δ=8.10 (s, 3H), 7.24 (dd, J=5.2, 8.4 Hz, 1H), 7.18-7.10 (m, 1H), 5.38-5.17 (m, 1H), 4.38 (s, 2H), 4.14-4.06 (m, 1H), 4.03-3.97 (m, 1H), 3.87-3.70 (m, 2H), 3.41 (s, 3H), 3.28 (s, 3H), 3.11 (s, 3H), 3.10-3.04 (m, 2H), 3.00 (s, 1H), 2.85-2.75 (m, 3H), 2.14-2.09 (m, 1H), 2.07-1.98 (m, 4H), 1.87-1.74 (m, 3H); LCMS (ESI, M+1): m/z=730.1.

### Example 308

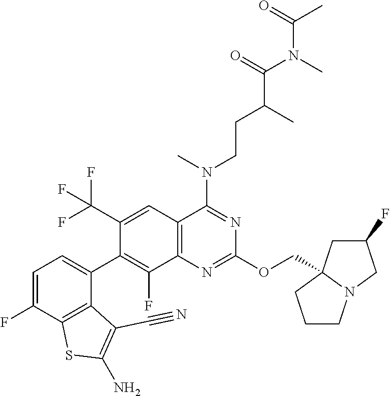 [SMILES: CC(=O)N(C)C(=O)C(C)CCN(C)c1nc(OC[C@@]23CCCN2C[C@H](F)C3)nc2c(F)c(-c3ccc(F)c4sc(N)c(C#N)c34)c(C(F)(F)F)cc12]

### N-acetyl-4-((7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(trifluoromethyl)quinazolin-4-yl)(methyl)amino)-N,2-dimethylbutanamide

### Example 309

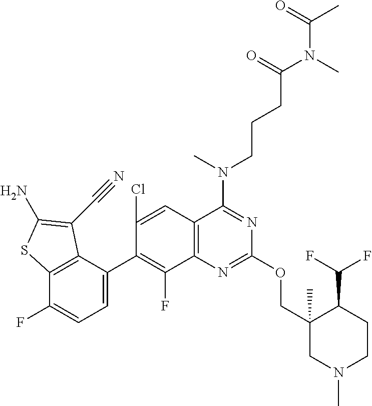 [SMILES: CC(=O)N(C)C(=O)CCCN(C)c1nc(OC[C@]2(C)CN(C)CC[C@@H]2C(F)F)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-2-(((3S,4S)-4-(difluoromethyl)-1,3-dimethylpiperidin-3-yl)methoxy)-8-fluoroquinazolin-4-yl)(methyl)amino)-N-methylbutanamide

### Example 310

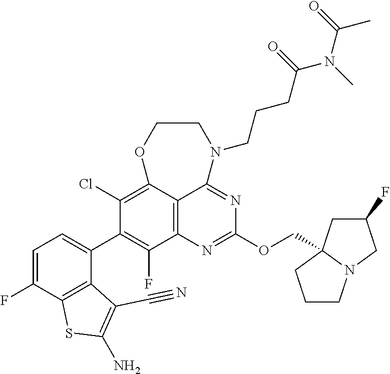 [SMILES: CC(=O)N(C)C(=O)CCCN1CCOc2c(Cl)c(-c3ccc(F)c4sc(N)c(C#N)c34)c(F)c3nc(OC[C@@]45CCCN4C[C@H](F)C5)nc1c23]

### N-acetyl-4-(9-(2-amino-3-cyano-7-fluorobenzo[b]thiophen-4-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino [5,6,7-de]quinazolin-4-yl)-N-methylbutanamide EXAMPLE 311

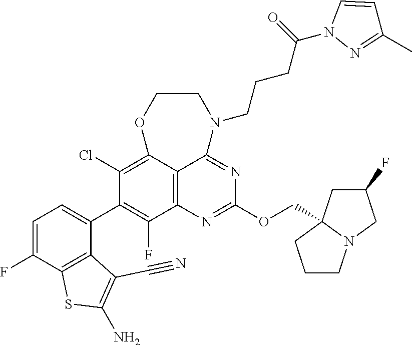 [SMILES: Cc1ccn(C(=O)CCCN2CCOc3c(Cl)c(-c4ccc(F)c5sc(N)c(C#N)c45)c(F)c4nc(OC[C@@]56CCCN5C[C@H](F)C6)nc2c34)n1]

### 2-amino-4-(8-chloro-10-fluoro-2-(((2R,7aS)-2-fluorotetrahydro-1H-pyrrolizin-7a(5H)-yl)methoxy)-4-(4-(3-methyl-1H-pyrazol-1-yl)-4-oxobutyl)-5,6-dihydro-4H-[1,4]oxazepino [5,6,7-de]quinazolin-9-yl)-7-fluorobenzo[b]thiophene-3-carbonitrile

### Example 312

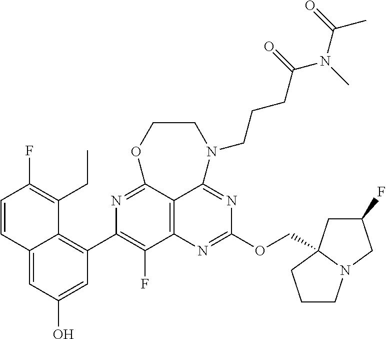 [SMILES: CCc1c(F)ccc2cc(O)cc(-c3nc4c5c(nc(OC[C@@]67CCCN6C[C@H](F)C7)nc5c3F)N(CCCC(=O)N(C)C(C)=O)CCO4)c12]

### N-acetyl-4-(5-(8-ethyl-7-fluoro-3-hydroxynaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta [de]naphthalen-10-yl)-N-methylbutanamide

### Example 313

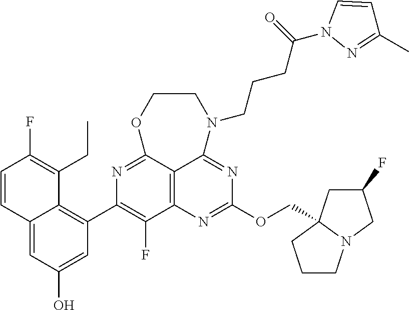 [SMILES: CCc1c(F)ccc2cc(O)cc(-c3nc4c5c(nc(OC[C@@]67CCCN6C[C@H](F)C7)nc5c3F)N(CCCC(=O)n3ccc(C)n3)CCO4)c12]

### 4-(5-(8-ethyl-7-fluoro-3-hydroxynaphthalen-1-yl)-4-fluoro-2-(((2R,7aS)-2-fluorotetrahydro-1H-pyrrolizin-7a(5H)-yl)methoxy)-8,9-dihydro-10H-7-oxa-1,3,6,10-tetraazacyclohepta [de]naphthalen-10-yl)-1-(3-methyl-1H-pyrazol-1-yl) butan-1-one

### Example 314

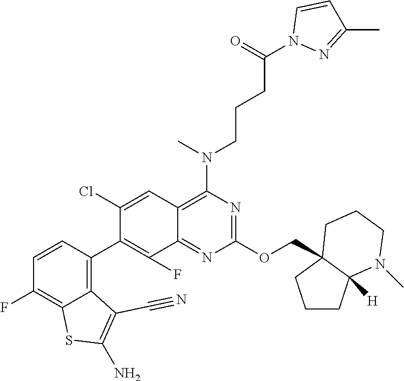 [SMILES: Cc1ccn(C(=O)CCCN(C)c2nc(OC[C@]34CCC[C@H]3N(C)CCC4)nc3c(F)c(-c4ccc(F)c5sc(N)c(C#N)c45)c(Cl)cc23)n1]

### 2-amino-4-(6-chloro-8-fluoro-4-(methyl (4-(3-methyl-1H-pyrazol-1-yl)-4-oxobutyl)amino)-2-(((4aS,7aR)-1-methyloctahydro-4aH-cyclopenta[b]pyridin-4a-yl)methoxy)quinazolin-7-yl)-7-fluorobenzo[b]thiophene-3-carbonitrile

### Example 315

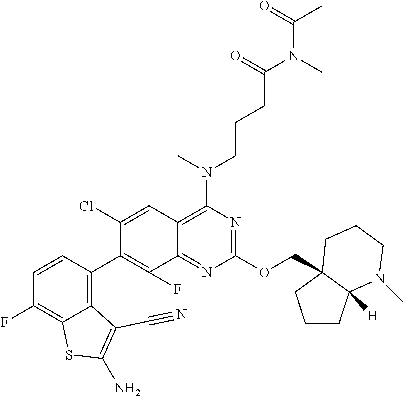 [SMILES: CC(=O)N(C)C(=O)CCCN(C)c1nc(OC[C@]23CCC[C@H]2N(C)CCC3)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-8-fluoro-2-(((4aS,7aR)-1-methyloctahydro-4aH-cyclopenta[b]pyridin-4a-yl)methoxy)quinazolin-4-yl)(methyl)amino)-N-methylbutanamide

### Example 316

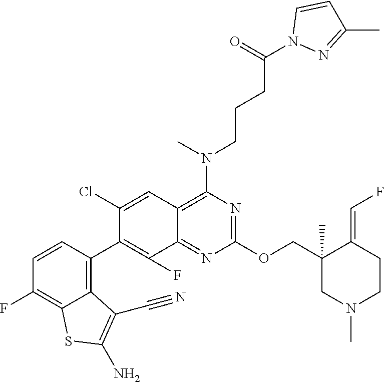 [SMILES: Cc1ccn(C(=O)CCCN(C)c2nc(OC[C@]3(C)CN(C)CC/C3=C\F)nc3c(F)c(-c4ccc(F)c5sc(N)c(C#N)c45)c(Cl)cc23)n1]

### 2-amino-4-(6-chloro-8-fluoro-2-(((S,E)-4-(fluoromethylene)-1,3-dimethylpiperidin-3-yl)methoxy)-4-(methyl (4-(3-methyl-1H-pyrazol-1-yl)-4-oxobutyl)amino)quinazolin-7-yl)-7-fluorobenzo[b]thiophene-3-carbonitrile

### Example 317

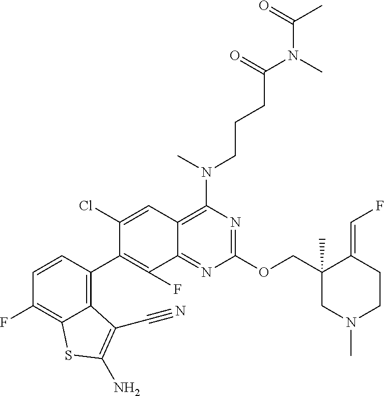 [SMILES: CC(=O)N(C)C(=O)CCCN(C)c1nc(OC[C@]2(C)CN(C)CC/C2=C\F)nc2c(F)c(-c3ccc(F)c4sc(N)c(C#N)c34)c(Cl)cc12]

### N-acetyl-4-((7-(2-amino-3-cyano-7-fluorobenzo[b]thiophen-4-yl)-6-chloro-8-fluoro-2-(((S,E)-4-(fluoromethylene)-1,3-dimethylpiperidin-3-yl)methoxy)quinazolin-4-yl)(methyl)amino)-N-methylbutanamide

### Example 318

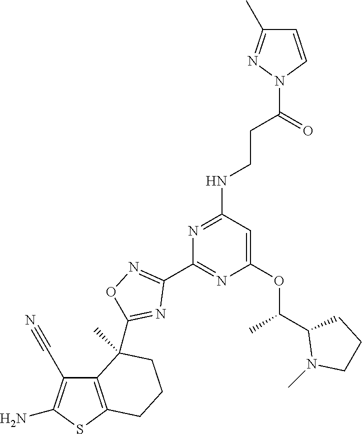 [SMILES: Cc1ccn(C(=O)CCNc2cc(O[C@@H](C)[C@@H]3CCCN3C)nc(-c3noc([C@@]4(C)CCCc5sc(N)c(C#N)c54)n3)n2)n1]

### (S)-2-amino-4-methyl-4-(3-(4-((3-(3-methyl-1H-pyrazol-1-yl)-3-oxopropyl)amino)-6-((S)-1-((S)-1-methylpyrrolidin-2-yl)ethoxy)pyrimidin-2-yl)-1,2,4-oxadiazol-5-yl)-4,5,6,7-tetrahydrobenzo[b]thiophene-3-carbonitrile

### Example 319

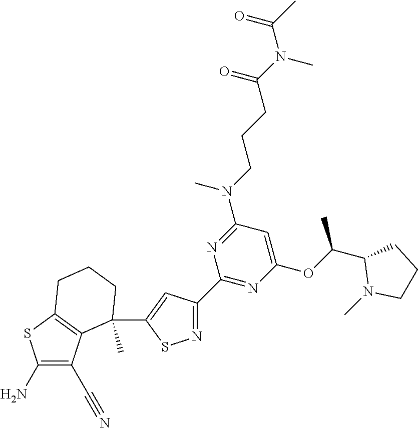 [SMILES: CC(=O)N(C)C(=O)CCCN(C)c1cc(O[C@@H](C)[C@@H]2CCCN2C)nc(-c2cc([C@]3(C)CCCc4sc(N)c(C#N)c43)sn2)n1]

### N-acetyl-4-((2-(5-((R)-2-amino-3-cyano-4-methyl-4,5,6,7-tetrahydrobenzo[b]thiophen-4-yl)isothiazol-3-yl)-6-((S)-1-((S)-1-methylpyrrolidin-2-yl)ethoxy)pyrimidin-4-yl)(methyl)amino)-N-methylbutanamide

### Example 320

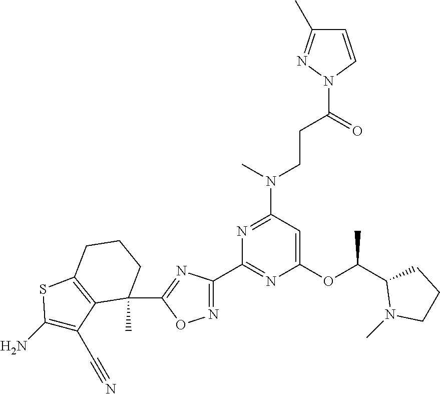 [SMILES: Cc1ccn(C(=O)CCN(C)c2cc(O[C@@H](C)[C@@H]3CCCN3C)nc(-c3noc([C@]4(C)CCCc5sc(N)c(C#N)c54)n3)n2)n1]

### (R)-2-amino-4-methyl-4-(3-(4-(methyl (3-(3-methyl-1H-pyrazol-1-yl)-3-oxopropyl)amino)-6-((S)-1-((S)-1-methylpyrrolidin-2-yl)ethoxy)pyrimidin-2-yl)-1,2,4-oxadiazol-5-yl)-4,5,6,7-tetrahydrobenzo[b]thiophene-3-carbonitrile

### Example 321

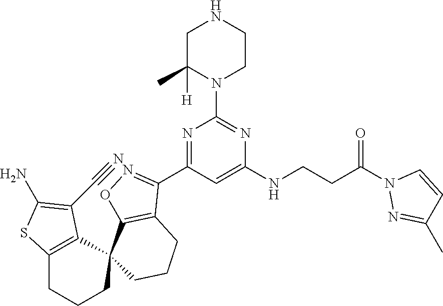 [SMILES: Cc1ccn(C(=O)CCNc2cc(-c3noc4c3CCC[C@@]43CCCc4sc(N)c(C#N)c43)nc(N3CCNC[C@@H]3C)n2)n1]

### (S)-2-amino-3′-(6-((3-(3-methyl-1H-pyrazol-1-yl)-3-oxopropyl)amino)-2-((S)-2-methylpiperazin-1-yl)pyrimidin-4-yl)-5′,6,6′,7-tetrahydro-4′H,5H-spiro[benzo[b]thiophene-4,7′-benzo[d]isoxazole]-3-carbonitrile EXAMPLE 322

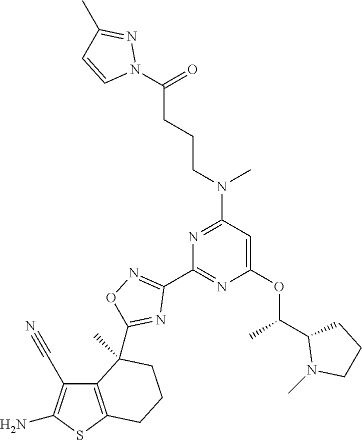 [SMILES: Cc1ccn(C(=O)CCCN(C)c2cc(O[C@@H](C)[C@@H]3CCCN3C)nc(-c3noc([C@@]4(C)CCCc5sc(N)c(C#N)c54)n3)n2)n1]

### (S)-2-amino-4-methyl-4-(3-(4-(methyl (4-(3-methyl-1H-pyrazol-1-yl)-4-oxobutyl)amino)-6-((S)-1-((S)-1-methylpyrrolidin-2-yl)ethoxy)pyrimidin-2-yl)-1,2,4-oxadiazol-5-yl)-4,5,6,7-tetrahydrobenzo[b]thiophene-3-carbonitrile

### Example 323

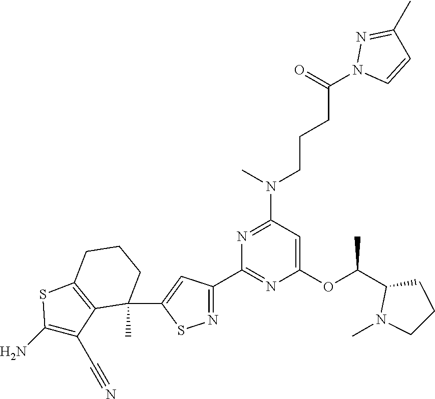 [SMILES: Cc1ccn(C(=O)CCCN(C)c2cc(O[C@@H](C)[C@@H]3CCCN3C)nc(-c3cc([C@]4(C)CCCc5sc(N)c(C#N)c54)sn3)n2)n1]

### (R)-2-amino-4-methyl-4-(3-(4-(methyl (4-(3-methyl-1H-pyrazol-1-yl)-4-oxobutyl)amino)-6-((S)-1-((S)-1-methylpyrrolidin-2-yl)ethoxy)pyrimidin-2-yl)isothiazol-5-yl)-4,5,6,7-tetrahydrobenzo[b]thiophene-3-carbonitrile

### Example 324

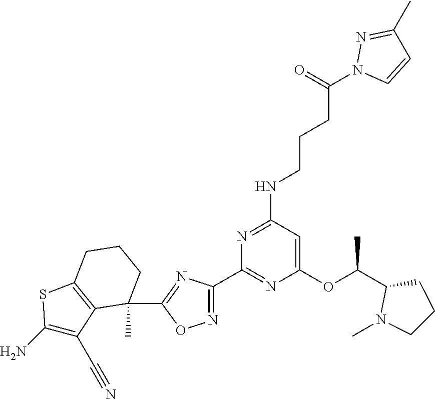 [SMILES: Cc1ccn(C(=O)CCCNc2cc(O[C@@H](C)[C@@H]3CCCN3C)nc(-c3noc([C@]4(C)CCCc5sc(N)c(C#N)c54)n3)n2)n1]

### (R)-2-amino-4-methyl-4-(3-(4-((4-(3-methyl-1H-pyrazol-1-yl)-4-oxobutyl)amino)-6-((S)-1-((S)-1-methylpyrrolidin-2-yl)ethoxy)pyrimidin-2-yl)-1,2,4-oxadiazol-5-yl)-4,5,6,7-tetrahydrobenzo[b]thiophene-3-carbonitrile

### Example 325

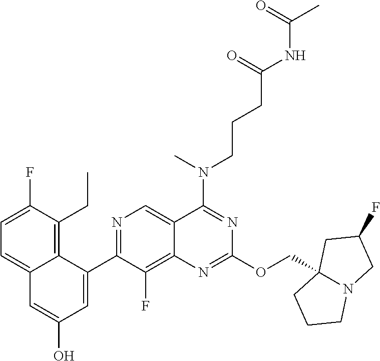 [SMILES: CCc1c(F)ccc2cc(O)cc(-c3ncc4c(N(C)CCCC(=O)NC(C)=O)nc(OC[C@@]56CCCN5C[C@H](F)C6)nc4c3F)c12]

### N-acetyl-4-((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)(methyl)amino)butanamide

### Example A: KRas Binding Assay

This Example illustrates that exemplary compounds of the present invention bind to KRas and are capable of displacing a labeled tracer ligand occupying the KRas binding site. KRas^{WT}, KRas^{G12A}, KRas^{G12C}, KRas^{G12D}, KRas^{G12R}, KRas^{G12S}, KRas^{G12V}, KRas^{G13D}, or KRas^{Q61H} was used in the assay.

The ability of a compound to bind to KRas was measured using a TR-FRET displacement assay. Biotinylated KRas (corresponding to amino acids 1-169, produced at Accelegan Inc.) was incubated with custom made Cy5 labelled tracer, terbium streptavidin (Cisbio Inc.) and compound (1% DMSO final) in buffer (50 mM HEPES, pH 7.5, 5 mM MgCl₂, 0.005% Tween-20 and 1 mM DTT). After a 60-minute incubation at room temperature, the reaction was measured using a BMG LABTECH CLARIO star Plus via TR-FRET. 100 percent of control (POC) is determined by using a DMSO control and 0 POC is determined using a concentration of control compound that completely inhibits binding of the tracer to KRas. The POC values were fit to a 4-parameter IC₅₀ equation and the IC₅₀ value reported.

TABLE 1
Binding to KRas (IC₅₀ nM) by Exemplary
Compounds of Formula (Ia) or (Ib)

| Ex. No. | G12S | G12C | WT |
|---|---|---|---|
| 1 | 36 | 77 | 63 |
| 2 | 72513 | >100000 | 73864 |
| 3 | 346 | 582 | 649 |
| 4 | 9 | 22 | 51 |
| 5 | 26466 | 7565 | 34400 |
| 6 | 7954 | 10583 | 8064 |
| 7 | 21225 | 20391 | 24170 |
| 8 | >100000 | >100000 | >100000 |
| 9 | 31508 | 30614 | 64635 |
| 10 | 4674 | 19641 | 43895 |
| 11 | 3843 | 8156 | 9274 |
| 12 | 59574 | 808 | 48528 |
| 13 | 39036 | 272 | 28697 |
| 14 | >100000 | 877 | 44973 |
| 15 | 9 | <2 | 11 |
| 16 | 44 | 54 | 23 |
| 17 | 15255 | 1075 | 5783 |
| 18 | 29996 | 42108 | 7974 |
| 19 | 7456 | 20059 | 2801 |
| 20 | 98762 | 27272 | 14692 |
| 21 | >100000 | 15874 | 52188 |
| 22 | >100000 | 95220 | 14116 |
| 23 | 61578 | 34788 | 11765 |
| 24 | >100000 | 30488 | 17678 |
| 25 | >100000 | 66889 | 10870 |
| 26 | 75598 | 92808 | 10632 |
| 27 | >100000 | 452 | 89211 |
| 28 | 74739 | 724 | >100000 |
| 29 | >100000 | 201 | 90499 |
| 30 | 32753 | 194 | >100000 |
| 31 | >100000 | >100000 | 39392 |
| 32 | 43274 | 128 | >100000 |
| 33 | >100000 | 10698 | >100000 |
| 34 | >100000 | 7890 | >100000 |
| 35 | 47470 | 321 | 92608 |
| 36 | >100000 | 18859 | >100000 |
| 37 | 20384 | 7290 | 20371 |
| 38 | 27946 | 761 | 19311 |
| 39 | 1588 | 13 | 195 |
| 40 | 1247 | 9 | 729 |
| 41 | 17642 | 18159 | 12277 |
| 42 | 68132 | 768 | >100000 |
| 43 | 42071 | 424 | >100000 |
| 44 | 81102 | 1771 | >100000 |
| 45 | >100000 | >100000 | 65674 |
| 46 | >100000 | >100000 | >100000 |
| 47 | >100000 | >100000 | >100000 |
| 48 | 30085 | 2742 | |
| 49 | 54779 | 65872 | |
| 50 | 55615 | 3445 | |
| 51 | 56497 | 30349 | 25394 |
| 52 | 27105 | 6812 | 20153 |
| 53 | 2 | 7 | 77 |
| 54 | 266 | 502 | 483 |
| 55 | 28 | 5 | 7 |
| 56 | 17 | 13 | 69 |
| 57 | 66 | 96 | 107 |
| 58 | 221 | 108 | 89 |
| 59 | <2 | 8 | <2 |
| 60 | 115 | 76 | 24 |
| 61 | 34 | 336 | 136 |
| 62 | 6334 | 619 | 64 |
| 63 | 20 | 96 | 145 |
| 64 | 40 | 13 | 32 |
| 65 | 14 | 28 | 17 |
| 66 | 26 | 57 | 154 |
| 67 | 17 | 51 | 15 |
| 68 | 6 | 5 | 10 |
| 69 | 124 | 79 | 6 |
| 70 | 17 | 31 | 27 |
| 71 | 2502 | 1688 | 2087 |
| 72 | 274 | 408 | 572 |
| 73 | 2955 | 888 | 1077 |
| 74 | 885 | 578 | 798 |
| 75 | 346 | 234 | 378 |
| 76 | 150 | 124 | 166 |
| 77 | 87 | 159 | 161 |
| 78 | 146 | 128 | 25 |
| 79 | 594 | 26 | 246 |
| 80 | 197 | 307 | 432 |
| 81 | 93 | 165 | 32 |
| 82 | 28 | 34 | 23 |
| 83 | 402 | 107 | 790 |
| 84 | 7 | 53 | 154 |
| 85 | 17 | 28 | 8 |
| 86 | 2203 | 3447 | 2315 |
| 87 | 1676 | 365 | 1581 |
| 88 | 1058 | 116 | 1761 |
| 89 | 6634 | 2474 | 8492 |
| 90 | 286 | 260 | 233 |
| 91 | 62 | 141 | 317 |
| 92 | 17 | 3 | 59 |
| 93 | 3 | 4 | 13 |
| 94 | 232 | 15 | 306 |
| 95 | 1600 | 257 | 1085 |
| 96 | 339 | 375 | 162 |
| 97 | 201 | 395 | 125 |
| 98 | 20 | 73 | 178 |
| 99 | 26 | 58 | 38 |
| 100 | 4716 | 4842 | 7690 |
| 101 | 934 | 784 | 716 |
| 102 | 2137 | 252 | 5921 |
| 103 | 35 | 37 | 34 |
| 104 | 176 | 295 | 77 |
| 105 | 50 | 35 | 58 |
| 106 | 14 | 22 | 40 |
| 107 | 513 | 37 | 1065 |
| 108 | 1321 | 339 | 1042 |
| 109 | 99 | 134 | 73 |
| 110 | <2 | <2 | 58 |
| 111 | 3 | <2 | 13 |
| 112 | 9 | 11 | 19 |
| 113 | 31 | 137 | 78 |
| 114 | 664 | 776 | 1180 |
| 115 | 1799 | 4125 | 260 |
| 116 | 11 | 111 | 3479 |
| 117 | 158 | 129 | 68 |
| 118 | 437 | 591 | 873 |
| 119 | 183 | 171 | 402 |
| 120 | 186 | 142 | 1213 |
| 121 | 16 | 16 | 30 |
| 122 | 5 | 9 | 19 |
| 123 | 68 | 42 | 30 |
| 124 | 3 | 4 | 103 |
| 125 | 168 | 96 | 84 |
| 126 | 88 | 84 | 8 |
| 127 | 407 | 224 | 193 |
| 128 | 11 | 93 | 1191 |
| 129 | 5 | 7 | 8 |
| 130 | 8 | 11 | 10 |
| 131 | 6 | 7 | 7 |
| 132 | 17 | 19 | 25 |
| 133 | 7 | 17 | 17 |
| 134 | 46 | 59 | 21 |
| 135 | 6 | 9 | 11 |
| 136 | 17 | 16 | 23 |
| 137 | 10 | 13 | 44 |
| 138 | 6 | 13 | 16 |
| 139 | 5 | 5 | 16 |
| 140 | 3 | 11 | 15 |
| 141 | <2 | <2 | 11 |
| 142 | <2 | <2 | 10 |
| 143 | 8 | 56 | 166 |
| 144 | 32 | 50 | 68 |
| 145 | 27 | 237 | 331 |
| 146 | 477 | 676 | 412 |
| 147 | 37 | 16 | 45 |
| 148 | 10 | 19 | 147 |
| 149 | 1473 | 3735 | 1312 |
| 150 | 3 | 5 | 2 |
| 151 | 18 | 12 | 9 |
| 152 | 84 | 73 | 39 |
| 153 | 642 | 439 | 138 |
| 154 | 257 | 338 | 131 |
| 155 | 135 | 204 | 140 |
| 156 | 15 | 89 | 585 |
| 157 | 122 | 84 | 32 |
| 158 | 23 | 29 | 13 |
| 159 | 4 | 6 | 15 |
| 160 | 461 | 1792 | 258 |
| 161 | 185 | 86 | 282 |
| 162 | 491 | 673 | 1054 |
| 163 | 129 | 131 | 98 |
| 164 | 8 | 5 | 103 |
| 165 | 4 | 2 | 52 |
| 166 | 203 | 310 | 229 |
| 167 | 29 | 94 | 528 |
| 168 | 72 | 384 | 1380 |
| 170 | 2 | 2 | 9 |
| 173 | 27 | 29 | 31 |
| 174 | 182 | 1120 | 1123 |
| 175 | 8 | 9 | 16 |
| 176 | 5 | 3 | 12 |
| 177 | 4 | 2 | 9 |
| 178 | 15 | 13 | 44 |
| 179 | 42 | 64 | 26 |
| 180 | 4 | 26 | 67 |
| 181 | 9 | 10 | 17 |
| 182 | 38 | 24 | 35 |
| 183 | 3 | 4 | 5 |
| 184 | 6 | 5 | 27 |
| 185 | 12 | 18 | 6 |
| 186 | 156 | 136 | 32 |
| 187 | 33 | 40 | 11 |
| 188 | 13 | 8 | 26 |
| 189 | 8 | 13 | 24 |
| 190 | 4 | 5 | 13 |
| 191 | 7 | <2 | 6 |
| 192 | 35 | 27 | 23 |
| 193 | 11 | 5 | 113 |
| 194 | 7 | 4 | 209 |
| 195 | 6 | 13 | 43 |
| 196 | 45 | 40 | 55 |
| 197 | 28 | 21 | 176 |
| 198 | 14 | 2 | 77 |
| 199 | 27 | 17 | 26 |
| 200 | 4 | 2 | 5 |
| 201 | 5 | 4 | 6 |
| 202 | 2 | <2 | 4 |
| 203 | 3 | <2 | 4 |
| 204 | 13 | <2 | 38 |
| 205 | 4 | 2 | 7 |
| 206 | 9 | 7 | 8 |
| 207 | 66 | 22 | 238 |
| 208 | 74 | 4 | 171 |
| 209 | 165 | 168 | 496 |
| 210 | 1294 | 2251 | 2734 |
| 211 | 15 | 61 | 314 |
| 212 | 371 | 623 | 165 |
| 213 | 5 | <2 | 8 |
| 214 | 13 | 8 | 14 |
| 215 | 169 | 2397 | 11354 |
| 216 | 3 | 4 | 22 |
| 217 | 7 | 5 | 9 |
| 218 | 16 | 3 | 83 |
| 219 | 4 | 3 | 10 |
| 220 | 153 | 209 | 160 |
| 221 | 11 | 43 | 101 |
| 222 | 3414 | 18302 | 29629 |
| 223 | 53 | 42 | 164 |
| 224 | 257 | 526 | 177 |
| 225 | 18 | 6 | 30 |
| 226 | 8 | <2 | 6 |
| 227 | 13 | 19 | 93 |
| 228 | 8 | 3 | 13 |
| 229 | 631 | 586 | 161 |
| 230 | 123 | 134 | 91 |
| 231 | 17 | 5 | 13 |
| 232 | 7 | 3 | 12 |
| 233 | 8 | 6 | 11 |
| 234 | 3 | <2 | 9 |
| 235 | 2 | 3 | 6 |
| 236 | 15 | 7 | 10 |
| 237 | 6 | 18 | 15 |
| 238 | 14 | 11 | 10 |
| 239 | 8 | 6 | 11 |
| 240 | 62 | 66 | 16 |
| 241 | 11 | 11 | 5 |
| 242 | 12 | 14 | 8 |
| 243 | 127 | 19 | 330 |
| 244 | 23 | 9 | 289 |
| 245 | 8 | 12 | 6 |
| 246 | 14 | 13 | 8 |
| 247 | 24 | 21 | 12 |
| 248 | 57 | 360 | 669 |
| 249 | 7 | <2 | 11 |
| 250 | 34 | <2 | 37 |
| 251 | 65 | 21 | 70 |
| 252 | 77 | 48 | 39 |
| 253 | 8 | 8 | 10 |
| 254 | 3 | 3 | 4 |
| 255 | 11 | 14 | 14 |
| 256 | 2 | 5 | 4 |
| 257 | 499 | 5 | 214 |
| 258 | 4 | 3 | 9 |
| 259 | 3 | 3 | 8 |
| 260 | <2 | <2 | 4 |
| 261 | 15 | 14 | 23 |
| 262 | 25 | 15 | 25 |
| 263 | 490 | 1718 | 2132 |
| 264 | 232 | 170 | 732 |
| 265 | 12 | 3 | 35 |
| 266 | 9 | 5 | 10 |
| 267 | 8 | 5 | 9 |
| 268 | 9 | 5 | 11 |
| 269 | 6 | 4 | 9 |
| 270 | 22 | 4 | 74 |
| 271 | 16 | 4 | 81 |
| 272 | 5 | 6 | 9 |
| 273 | 40 | 30 | 58 |
| 274 | 89 | 65 | 51 |
| 275 | 15 | 52 | 47 |
| 276 | 68 | 129 | 148 |
| 277 | 539 | 864 | 357 |
| 278 | 48 | 58 | 41 |
| 279 | 188 | 334 | 332 |
| 280 | 14 | 17 | 9 |
| 281 | 10 | 9 | 8 |
| 282 | 44 | 82 | 184 |
| 283 | 134 | 183 | 103 |
| 284 | 5 | 8 | 77 |
| 285 | 175 | 882 | 492 |
| 286 | 15 | 11 | 6 |
| 287 | 16 | 6 | 170 |
| 288 | 20 | 9 | 143 |
| 289 | 18 | 10 | 5 |
| 290 | 13 | 9 | 10 |
| 291 | 7 | 6 | 12 |
| 292 | 5 | 3 | 11 |
| 293 | 21 | 72 | 219 |
| 294 | 22 | 61 | 130 |
| 295 | 12 | 11 | 44 |
| 296 | 3 | 3 | 30 |
| 297 | 45 | 23 | 99 |
| 298 | 80 | 17 | 154 |
| 299 | 22 | 9 | 178 |
| 300 | 85 | 9 | 163 |
| 301 | 43 | 16 | 142 |
| 302 | 25 | 12 | 164 |
| 303 | 3 | 3 | 4 |
| 304 | 4 | 5 | 12 |
| 305 | 17 | 12 | 21 |
| 306 | 7 | 5 | 10 |
| 307 | 2 | 3 | 8 |
| 318 | 3 | 2 | 12 |
| 319 | >100000 | >100000 | >100000 |
| 320 | 179 | 131 | 264 |
| 321 | 21 | 14 | 28 |
| 322 | 20 | 10 | 525 |
| 323 | 26 | 17 | 346 |
| 324 | <2 | <2 | 19 |
| 325 | 14 | 12 | 23 |

### Example B

### Inhibition of KRas Phosphorylation of ERK (HTRF) by Exemplary Compounds of Formula (Ia) or (Ib)

Cisbio HTRF Advanced pERK Assay Catalog #64AERPEH
Cells: MKN1, PSN1
Procedure:
   1. Day 1: Seed 6,000 cells/well −25 μl/well in 384-well white solid bottom plate; RPM1_10% FBS. Incubate overnight at 37 °C/5% CO2.
   2. Day 2: Echo transfer 25 nl of 10 mM compound 10 point dilution at 1:3 (Cf=10 uM) and incubate for 3 hour at 37 °C/5% CO2.
      Add 8.5 μl/well of 4× Lysis Buffer/25× Blocking reagent (do not dump media) and incubate for 30 min at room temperature on shaker.
      Add conjugate mixture of 4.25 ul/well 1×-pERK-D2 and 1×-pERK-K diluted in Detection Buffer for a total of 8.5 μl/well.
      Incubate for 4 hours at room temperature covered.
      Read HTRF using ClarioStar
Cells: ASPC1, H727, A549, H460, HCT116, H358
Culture/Assay media: RPMI-1640+10% FBS

### Procedure:

### Cell Seeding

1. To harvest cells from flask using 0.05% Trypsin/EDTA solution. Add 10 mL of media to stop trypsinizing. Pipette the cells into a conical bottom 50 mL centrifuge tube and centrifuge 5 min×1000 rpm.
2. Re-suspend the cell pellet in media, take a cell count, and then adjust the cell density using fresh media.
3. Seed 6,000 cells into cell culture plate with 50 μL media. The
4. Incubate cell plate overnight in a 37 °C, 5% CO2 incubator.

### Compound Titrations

1. Use Tecan to complete the compound addition. Compounds start from 10 uM top, 3-fold dilution, and 10 doses. The final DMSO concentration is 0.8%. Dispensed 0.2 uM Trametinib as Min control.
2. Incubate cell plate for 3 hrs in the incubator.
   Detection with Cisbio pERK HTRF Kit
1. Dilute 1 volume of 4× lysis buffer with 3 volumes of deionized water. Then, add 100× the blocking reagent. Keep lysis buffer on the ice.
2. At the end of the compound treatment, flick-off the media.
3. Add 35 μL of lysis buffer per well using a Multidrop Combi. Then place on a plate agitator shaking at 300 rpm at 4 °C for 40 mins.
4. Make up the HTRF antibody buffer. For each assay plate, mix 50 μL of d2-conjugate antibody with 950 μL of detection buffer. Similarly, mix 50 μL of Cryptate antibody with 950 μL of detection buffer. Then mix the two diluted antibodies together.
5. Dispense 3.4 μL the antibody buffer to wells of an empty assay plate. Seal the plate and centrifuge plate 30 sec×1000 rpm.
6. At the end of the 4 °C lysis, centrifuge the lysate plates 3 mins×1500 rpm.
7. Use the Bravo to transfer 13.6 μL of lysate from cell culture plate to assay plate. Then incubate assay plate for 2 hrs at room temperature.
8. At the end of incubation, read plate on the Envision after centrifuging plate 30 sec×1000 rpm.

TABLE 2
Inhibition (HTRF IC₅₀ nM) of KRas -mediated Phosphorylation
of ERK by Exemplary Compounds of Formula (Ia) or (Ib)

| | A549 | H358 | MKN1 |
|---|---|---|---|
| Ex. No. | (G12S) | (G12C) | (WT) |
| 1 | 109 | 3725 | 3996 |
| 4 | 69 | 5441 | 2038 |
| 15 | 149 | 22 | |
| 16 | 337 | 428 | 70 |
| 39 | 6550 | 808 | 1358 |
| 40 | >10000 | 913 | |
| 53 | 16 | 88 | 305 |
| 54 | 334 | 1424 | 2420 |
| 55 | 116 | 90 | 50 |
| 56 | 66 | 2801 | |
| 57 | 200 | 5578 | 8499 |
| 58 | 338 | 677 | 427 |
| 59 | 372 | 486 | 27 |
| 60 | 2352 | 450 | |
| 61 | 84 | 8568 | 2610 |
| 62 | 7064 | 8387 | |
| 63 | 40 | 3251 | 1457 |
| 64 | 119 | 4604 | 4505 |
| 65 | >10000 | 415 | 266 |
| 66 | 141 | 9022 | 5204 |
| 67 | 113 | 3085 | 3286 |
| 68 | 77 | 34 | 12 |
| 69 | 4806 | 1121 | 31 |
| 70 | 85 | 7286 | 5095 |
| 71 | 2034 | 930 | 8475 |
| 72 | 748 | 7291 | 3331 |
| 73 | 8478 | 4505 | 5572 |
| 74 | 464 | 2291 | 3332 |
| 75 | 312 | 3111 | 1389 |
| 76 | 223 | 4721 | 2057 |
| 77 | >10000 | 1362 | 4556 |
| 78 | 3820 | 1253 | 90 |
| 79 | 785 | 23 | 1448 |
| 80 | 431 | 9703 | 3378 |
| 81 | 3774 | 2157 | 192 |
| 82 | 3408 | 499 | 179 |
| 83 | 1173 | 1412 | 2991 |
| 84 | 55 | 7602 | 6393 |
| 85 | 883 | 458 | 48 |
| 86 | 8959 | | 4231 |
| 87 | >10000 | | 4065 |
| 88 | 589 | | 8071 |
| 89 | >10000 | | >10000 |
| 90 | >10000 | | 9187 |
| 91 | 210 | | 853 |
| 92 | 1378 | | >10000 |
| 93 | 26 | | 325 |
| 94 | 1069 | | 6198 |
| 95 | 4222 | | >10000 |
| 96 | >10000 | | >10000 |
| 97 | 2118 | | 2854 |
| 98 | 61 | | 2485 |
| 99 | 54 | | 1048 |
| 100 | >10000 | | >10000 |
| 101 | >10000 | | 5548 |
| 102 | 1935 | | >10000 |
| 103 | 167 | | 351 |
| 104 | 1579 | | 931 |
| 105 | >10000 | | 517 |
| 106 | 584 | | 529 |
| 107 | 2067 | | >10000 |
| 108 | 1999 | | >10000 |
| 109 | 1919 | | 316 |
| 110 | 12 | 91 | 1317 |
| 111 | 16 | | 219 |
| 112 | 15 | | 80 |
| 113 | 110 | | 3145 |
| 114 | >10000 | | 7832 |
| 115 | >10000 | | 2671 |
| 116 | 35 | | 4439 |
| 117 | 4053 | | 333 |
| 118 | 7840 | | 5745 |
| 119 | >10000 | | >10000 |
| 120 | 229 | | 5378 |
| 121 | 243 | 382 | 875 |
| 122 | 38 | | 342 |
| 123 | 2736 | 264 | 219 |
| 124 | 7 | 25 | 93 |
| 125 | 6812 | | |
| 126 | 9804 | | |
| 127 | 7193 | 635 | 514 |
| 128 | 57 | 1540 | 7428 |
| 129 | 4 | 26 | 139 |
| 130 | 7 | 7 | 200 |
| 131 | 5 | 9 | 258 |
| 132 | >10000 | 1203 | 404 |
| 133 | 7 | 20 | 105 |
| 134 | 1844 | | 187 |
| 135 | 6 | | 83 |
| 136 | 249 | | 13 |
| 137 | 29 | | 142 |
| 138 | 137 | 157 | 106 |
| 139 | 46 | 412 | 1211 |
| 140 | 8 | 22 | 178 |
| 141 | 10 | 26 | 363 |
| 142 | 7 | 8 | 531 |
| 143 | 38 | 931 | |
| 144 | 5896 | 178 | |
| 145 | 137 | 2812 | 4006 |
| 146 | >10000 | 3826 | 2528 |
| 147 | 1207 | | 45 |
| 148 | 140 | | 108 |
| 149 | >10000 | | 6534 |
| 150 | 1581 | 39 | 40 |
| 151 | 1214 | 14 | 72 |
| 152 | 2182 | 83 | 251 |
| 153 | 4128 | 1758 | |
| 154 | 6275 | 7231 | 2868 |
| 155 | 445 | 2533 | 671 |
| 156 | 38 | 2355 | 2087 |
| 157 | 7589 | 2107 | 955 |
| 158 | 126 | | 112 |
| 159 | 6 | 108 | 57 |
| 160 | 3385 | | 3688 |
| 161 | 717 | >10000 | 7283 |
| 162 | 6484 | >10000 | >10000 |
| 163 | 7594 | | 1252 |
| 164 | 50 | 999 | 2211 |
| 165 | 62 | 450 | 226 |
| 166 | 1781 | | 622 |
| 167 | 326 | 5104 | 3048 |
| 168 | 245 | | 8686 |
| 169 | 534 | | 9481 |
| 170 | 2 | 3 | 53 |
| 171 | 6441 | | 3037 |
| 172 | 35 | 50 | 199 |
| 173 | 772 | | 54 |
| 174 | 507 | | 3000 |
| 175 | 6 | 11 | 177 |
| 176 | 388 | 295 | 250 |
| 177 | 227 | 1115 | 1254 |
| 178 | 108 | 226 | 356 |
| 179 | 167 | | 110 |
| 180 | 27 | 232 | 441 |
| 181 | 15 | 71 | 111 |
| 182 | 179 | | 247 |
| 183 | 143 | | 192 |
| 184 | 115 | | 1499 |
| 185 | 250 | | 65 |
| 186 | 2835 | 1117 | 184 |
| 187 | 1365 | | 127 |
| 188 | 39 | 9 | 140 |
| 189 | 27 | 43 | 199 |
| 190 | 7 | 7 | 205 |
| 191 | 290 | 1 | 15 |
| 192 | 4246 | 160 | 105 |
| 193 | 230 | | 2143 |
| 194 | 130 | | 766 |
| 195 | 84 | 1168 | 936 |
| 196 | >10000 | | 2199 |
| 197 | 164 | | 4946 |
| 198 | 489 | 28 | 159 |
| 199 | 4325 | 432 | 236 |
| 200 | 729 | 112 | 86 |
| 201 | 1570 | 373 | 251 |
| 202 | 1483 | 23 | 142 |
| 203 | 195 | 1 | 101 |
| 204 | 351 | 17 | 230 |
| 205 | 25 | 59 | 540 |
| 206 | 82 | 31 | 26 |
| 207 | 6650 | 34 | 1393 |
| 208 | 3975 | 4 | 854 |
| 209 | 281 | 1460 | 1113 |
| 210 | 741 | 8656 | 5015 |
| 211 | 23 | 518 | 567 |
| 212 | >10000 | 4604 | 868 |
| 213 | 1178 | 10 | 387 |
| 214 | 661 | 93 | 120 |
| 215 | 242 | >10000 | 3915 |
| 216 | 2 | 5 | 71 |
| 217 | 6880 | 296 | 763 |
| 218 | 175 | 15 | 322 |
| 219 | 12 | 16 | 431 |
| 220 | 336 | 449 | 258 |
| 221 | 26 | 485 | 409 |
| 222 | 1967 | >10000 | 8260 |
| 223 | 2395 | 17 | 569 |
| 224 | 3772 | 1327 | 321 |
| 225 | 2725 | 13 | 237 |
| 226 | 69 | 1 | 4 |
| 227 | 46 | 185 | 522 |
| 228 | 448 | 9 | 252 |
| 229 | >10000 | >10000 | 333 |
| 230 | 4350 | 1498 | 394 |
| 231 | 444 | 4 | 25 |
| 232 | 234 | 3 | 97 |
| 233 | 278 | | 83 |
| 234 | 3 | 7 | 161 |
| 235 | 7 | 60 | 82 |
| 236 | 104 | 108 | 41 |
| 237 | 17 | 69 | 70 |
| 238 | 145 | 84 | 40 |
| 239 | 669 | 114 | 171 |
| 240 | 775 | 658 | 44 |
| 241 | 367 | 163 | 27 |
| 242 | 240 | 149 | 17 |
| 243 | 215 | 26 | 736 |
| 244 | 55 | 12 | 279 |
| 245 | 148 | 148 | 15 |
| 246 | 223 | 167 | 24 |
| 247 | 677 | 296 | 61 |
| 248 | 100 | 4216 | 2917 |
| 249 | 249 | 2 | 223 |
| 250 | 1681 | 8 | 357 |
| 251 | 5437 | 66 | 1285 |
| 252 | 806 | 391 | 156 |
| 253 | 14 | 61 | 37 |
| 254 | 5 | 29 | 18 |
| 255 | 28 | 29 | 41 |
| 256 | 3 | 8 | 19 |
| 257 | 677 | 21 | 726 |
| 258 | 29 | 38 | 84 |
| 259 | 19 | 80 | 125 |
| 260 | 6 | 18 | 36 |
| 261 | 67 | 159 | 159 |
| 262 | 381 | 1171 | 593 |
| 263 | 495 | >10000 | 8923 |
| 264 | 138 | 847 | 3490 |
| 265 | 271 | 370 | 844 |
| 266 | 410 | 58 | 236 |
| 267 | 653 | 230 | 354 |
| 268 | 266 | 75 | 205 |
| 269 | 1181 | 411 | 388 |
| 270 | 462 | 77 | 340 |
| 271 | 139 | 31 | 177 |
| 272 | 4 | 3 | 62 |
| 273 | 745 | 494 | 340 |
| 274 | 326 | 662 | 287 |
| 275 | 58 | 164 | 437 |
| 276 | 101 | 1482 | 1033 |
| 277 | 549 | 6508 | 2357 |
| 278 | 292 | 1177 | 217 |
| 279 | 276 | 4900 | 1694 |
| 280 | 107 | 428 | 102 |
| 281 | 243 | 198 | 41 |
| 282 | 185 | 1292 | 870 |
| 283 | 701 | 1321 | 323 |
| 284 | 7 | 39 | 446 |
| 285 | 151 | 4288 | 2423 |
| 286 | 237 | 106 | 24 |
| 287 | 104 | 13 | 360 |
| 288 | 77 | 24 | 246 |
| 289 | 271 | 81 | 33 |
| 290 | 142 | 29 | 18 |
| 291 | 65 | 81 | 134 |
| 292 | 27 | 30 | 80 |
| 293 | 67 | | 699 |
| 294 | 74 | 4495 | 3871 |
| 295 | 14 | 5 | 56 |
| 296 | 16 | 15 | 318 |
| 297 | 101 | 32 | 299 |
| 298 | 169 | 16 | 217 |
| 299 | 93 | 12 | 323 |
| 300 | 115 | 9 | 408 |
| 301 | 113 | 20 | 264 |
| 302 | 79 | 18 | 286 |
| 303 | 59 | 14 | 4 |
| 304 | 3 | 3 | 60 |
| 305 | 21 | 19 | 78 |
| 306 | 92 | 45 | 134 |
| 307 | 5 | 15 | 70 |
| 318 | 17 | 908 | 801 |
| 319 | >10000 | >10000 | >10000 |
| 320 | 621 | >10000 | >10000 |
| 321 | 192 | 1060 | 861 |
| 322 | 96 | 9585 | 7313 |
| 323 | 140 | 3329 | 1858 |
| 324 | 9 | 262 | 651 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of Formula (Ia) or (Ib): 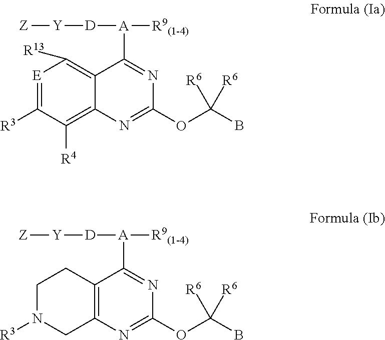
or a pharmaceutically acceptable salt thereof, wherein:
A is selected from: 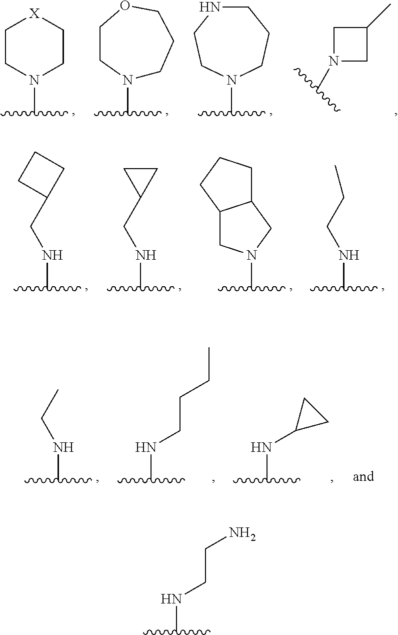
B is selected from: 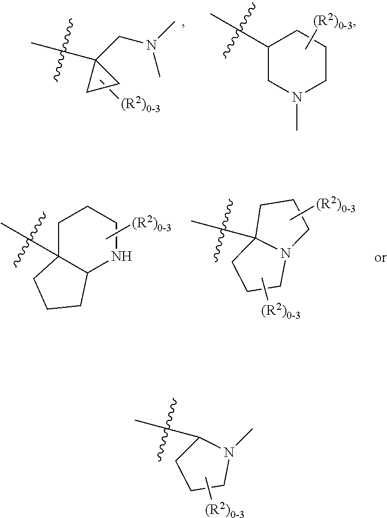
X is a bond, N, methylene, ethylene or propylene;
D is CO or SO₂;
Eis Nor CR¹⁰;
Y is a bond, O or NR⁵;
Z is hydrogen, C1-C4 alkyl, heterocyclyl, heteroaryl or aryl, where Z is optionally substituted with 1-3 substituents selected from halogen, C1-C4 alkyl, C1-C4 haloalkyl, OXO, OR¹², N(R¹²)₂, COR¹² and cyano;
each R² is independently hydrogen, hydroxy, halogen, C1-C3 alkyl, spiro cyclopropyl, C1-C3 cyanoalkyl, C1-C3 hydroxyalkyl, CHF₂, HC(═O)—, —OC(O)N(R⁵)₂, —CO₂R⁵, —CO₂N(R⁵)₂, ═CH₂, —CHR¹¹ or ═C(R¹¹)₂, wherein R² is optionally substituted with one or more R⁴;
R3 is aryl or heteroaryl, wherein the aryl or the heteroaryl is optionally substituted with one or more R⁸;
R⁴ is hydrogen, halogen or C1-C3 alkyl;
each R⁵ is independently hydrogen or C1-C3 alkyl;
each R⁶ is independently hydrogen, hydroxy, C1-C4 alkyl, C1-C4 hydroxyalkyl or heteroaryl, or two R⁶ join to form C3-C6 cycloalkyl or heterocycle;
each R⁸ is independently halogen, cyano, hydroxy, cycloalkyl, C1-C3 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, or —S—C1-C3 haloalkyl;
each R⁹ is independently hydrogen, C1-C4 alkyl, methoxy, CH₂-methoxy, hydroxy, CH₂F, CF₂, CF₃, C—CN, cyclopropyl, or two R⁹ on the same or different carbon atoms join to form a C1-C2 alkylene bridge or a spirocycle;
each R¹¹ is halogen or C1-C3 alkyl,
R¹² is C1-C3 alkyl, and
R¹³ is H or forms a ring with A.

2. The compound or salt according to claim 1, wherein Formula (Ia) is Formula IA:

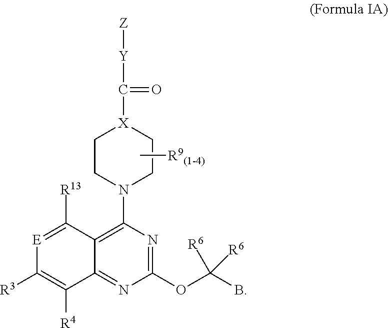

3. The compound or salt according to claim 1, wherein Formula (Ia) is Formula IB:

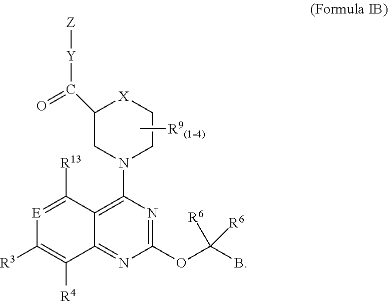

4. The compound or salt according to claim 1, wherein B is:

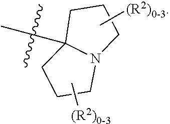

5-13. (canceled)

14. The compound or salt according to claim 1,
wherein Z is aryl, optionally substituted with 1-3 halogens, cyano, or C1-C4 haloalkyl.

15. The compound or salt of claim 14, wherein said aryl is phenyl.

16. The compound or salt according to claim 1,
wherein Z is heteroaryl, optionally substituted with 1-2 C1-C4 alkyl.

17. The compound or salt of claim 16, wherein said heteroaryl is pyrazolyl or pyridinyl.

18. The compound or salt according to claim 1,
wherein Z is heterocyclyl, optionally substituted with oxo.

19. The compound or salt of claim 18, wherein said heterocyclyl is oxazolidine.

20. The compound or salt according to claim 1,
wherein two R⁹ join to form a methylene bridge.

21. The compound or salt according to claim 1,
wherein two R⁹ join to form an ethylene bridge.

22. The compound or salt according to claim 4, wherein R² is fluoro.

23. The compound or salt according to claim 19, wherein R² is ═CHR¹¹.

24. The compound or salt according to claim 19, wherein R² is ═C(R¹¹)₂.

25-38. (canceled)

39. The compound or salt of claim 1, wherein the compound is selected from 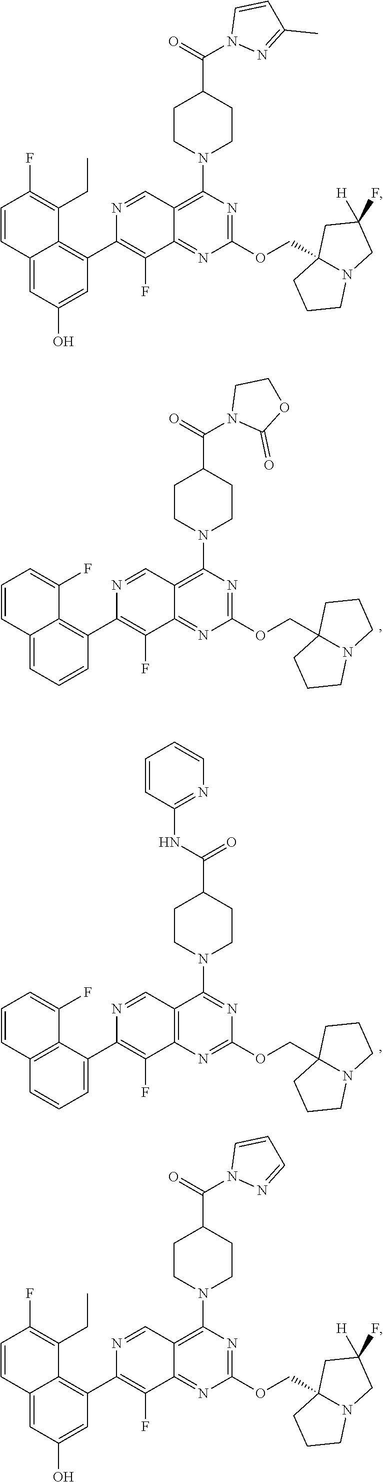 [SMILES: CCc1c(F)ccc2cc(O)cc(-c3ncc4c(N5CCC(C(=O)n6ccc(C)n6)CC5)nc(OC[C@@]56CCCN5C[C@H](F)C6)nc4c3F)c12 ; O=C1OCCN1C(=O)C1CCN(c2nc(OCC34CCCN3CCC4)nc3c(F)c(-c4cccc5cccc(F)c45)ncc23)CC1 ; O=C(Nc1ccccn1)C1CCN(c2nc(OCC34CCCN3CCC4)nc3c(F)c(-c4cccc5cccc(F)c45)ncc23)CC1 ; CCc1c(F)ccc2cc(O)cc(-c3ncc4c(N5CCC(C(=O)n6cccn6)CC5)nc(OC[C@@]56CCCN5C[C@H](F)C6)nc4c3F)c12] 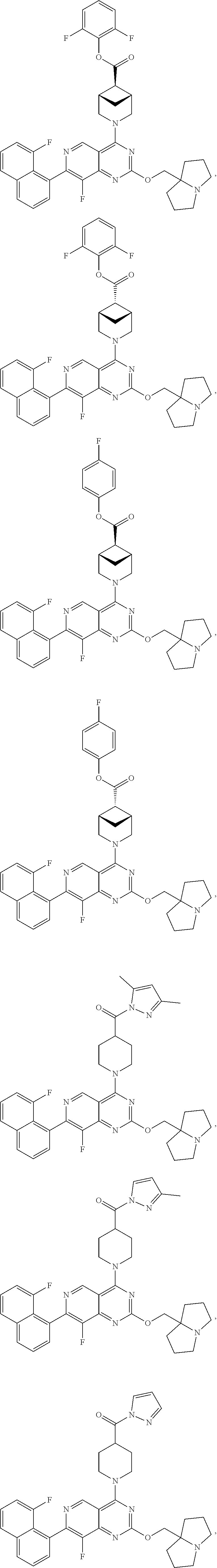 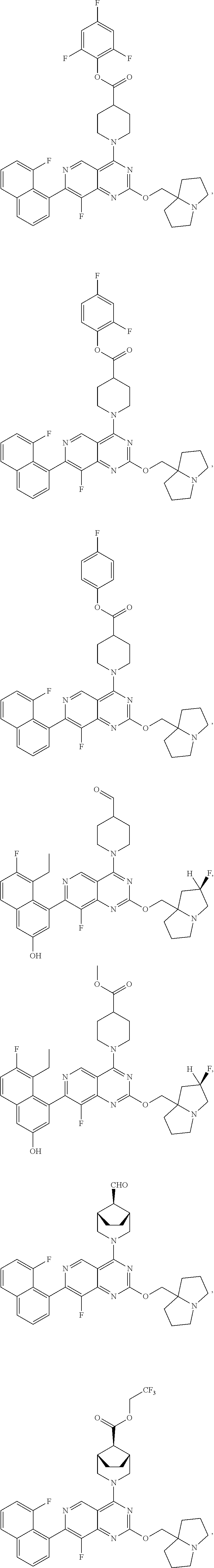 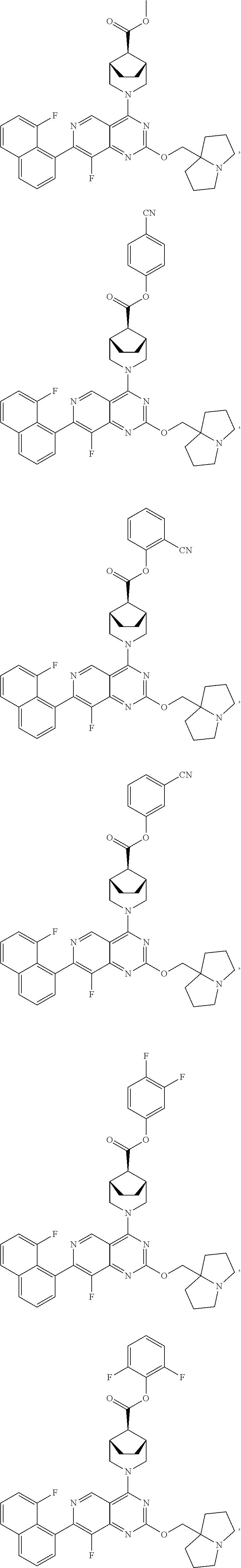 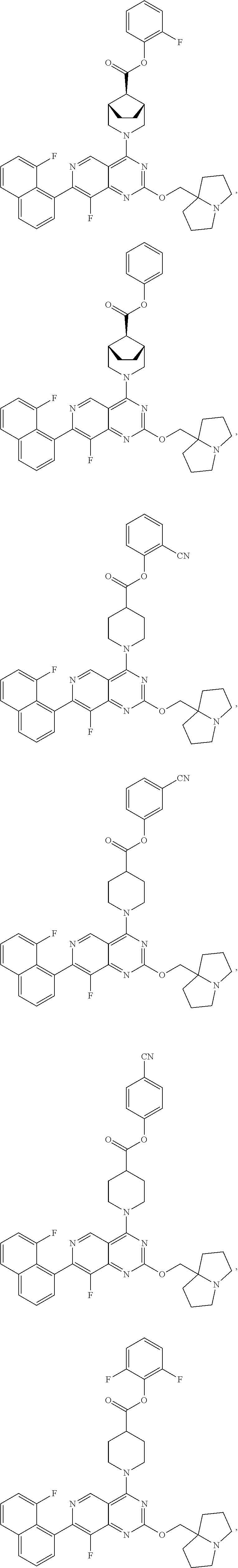 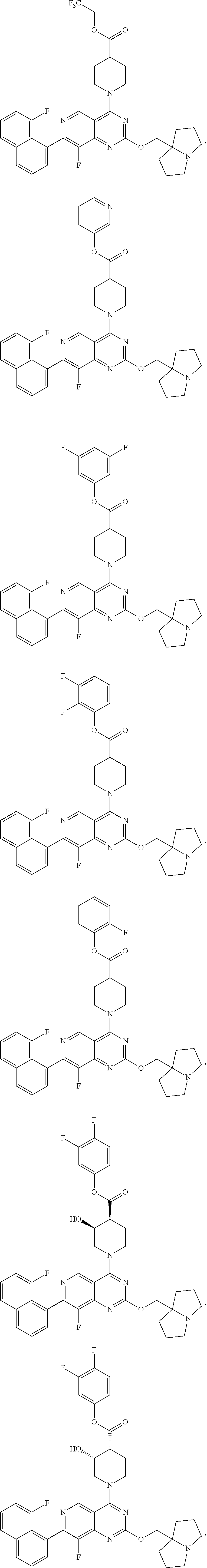 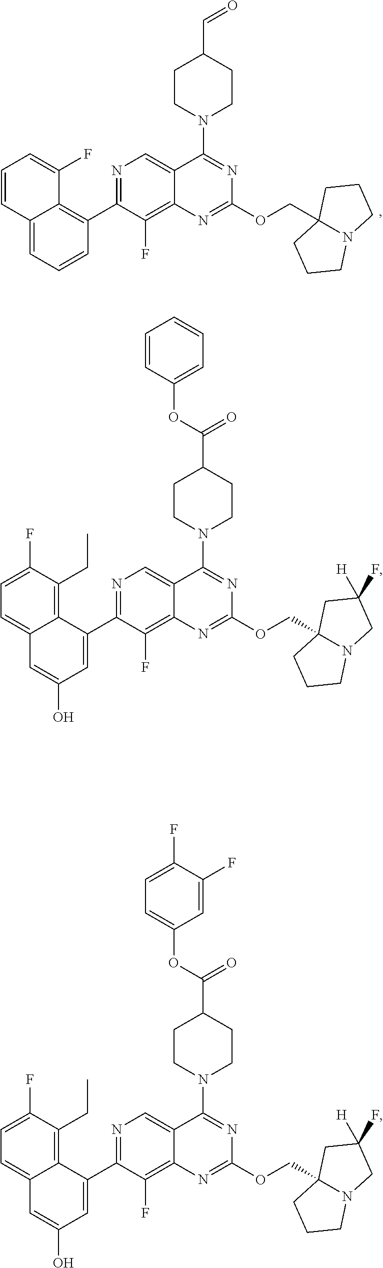 [SMILES: O=CC1CCN(c2nc(OCC34CCCN3CCC4)nc3c(F)c(-c4cccc5cccc(F)c45)ncc23)CC1 ; CCc1c(F)ccc2cc(O)cc(-c3ncc4c(N5CCC(C(=O)Oc6ccccc6)CC5)nc(OC[C@@]56CCCN5C[C@H](F)C6)nc4c3F)c12 ; CCc1c(F)ccc2cc(O)cc(-c3ncc4c(N5CCC(C(=O)Oc6ccc(F)c(F)c6)CC5)nc(OC[C@@]56CCCN5C[C@H](F)C6)nc4c3F)c12] 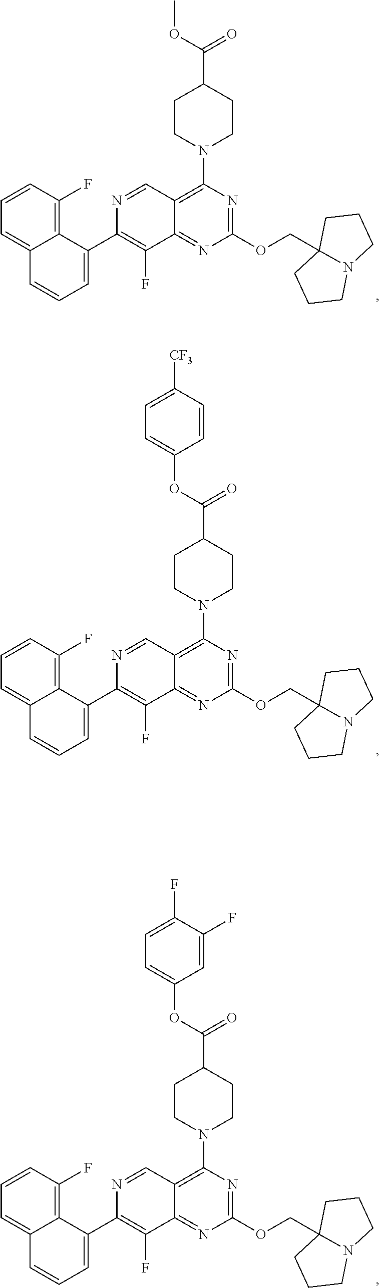 [SMILES: COC(=O)C1CCN(c2nc(OCC34CCCN3CCC4)nc3c(F)c(-c4cccc5cccc(F)c45)ncc23)CC1 ; O=C(Oc1ccc(C(F)(F)F)cc1)C1CCN(c2nc(OCC34CCCN3CCC4)nc3c(F)c(-c4cccc5cccc(F)c45)ncc23)CC1 ; O=C(Oc1ccc(F)c(F)c1)C1CCN(c2nc(OCC34CCCN3CCC4)nc3c(F)c(-c4cccc5cccc(F)c45)ncc23)CC1] 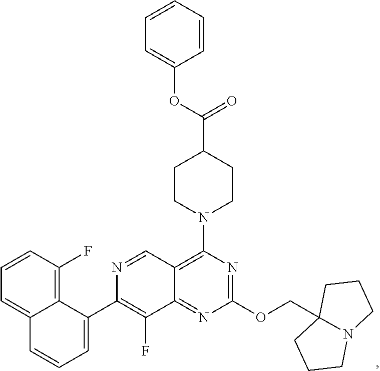 [SMILES: O=C(Oc1ccccc1)C1CCN(c2nc(OCC34CCCN3CCC4)nc3c(F)c(-c4cccc5cccc(F)c45)ncc23)CC1] 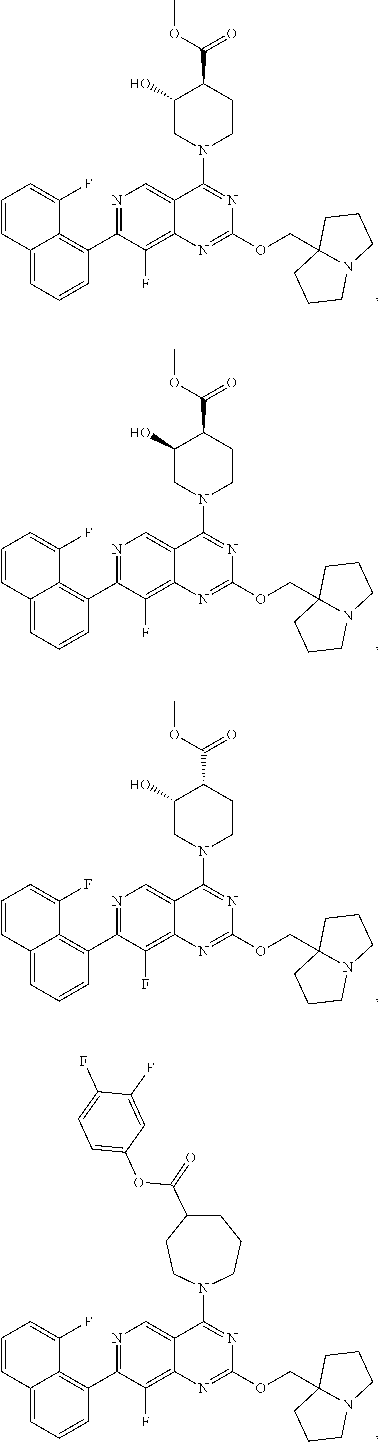 [SMILES: COC(=O)[C@H]1CCN(c2nc(OCC34CCCN3CCC4)nc3c(F)c(-c4cccc5cccc(F)c45)ncc23)C[C@@H]1O ; COC(=O)[C@H]1CCN(c2nc(OCC34CCCN3CCC4)nc3c(F)c(-c4cccc5cccc(F)c45)ncc23)C[C@H]1O ; COC(=O)[C@@H]1CCN(c2nc(OCC34CCCN3CCC4)nc3c(F)c(-c4cccc5cccc(F)c45)ncc23)C[C@@H]1O ; O=C(Oc1ccc(F)c(F)c1)C1CCCN(c2nc(OCC34CCCN3CCC4)nc3c(F)c(-c4cccc5cccc(F)c45)ncc23)CC1] 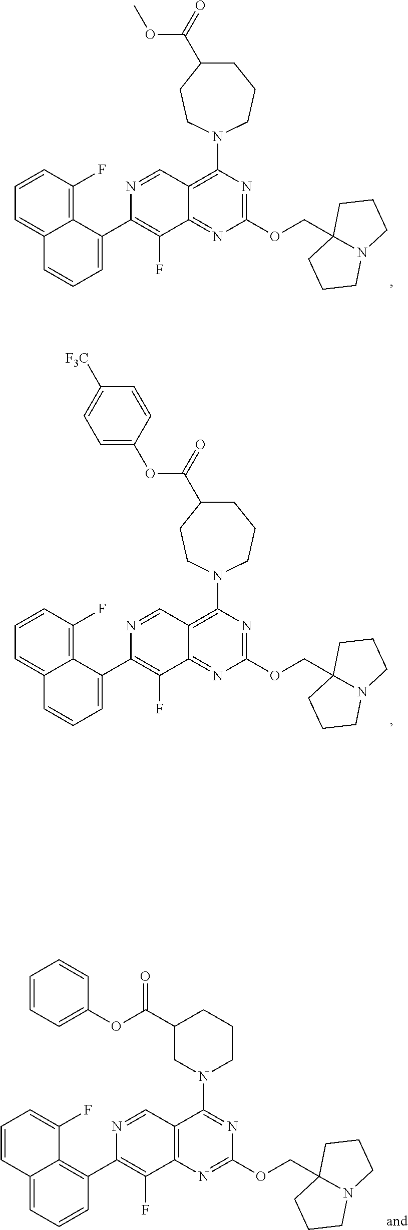 [SMILES: COC(=O)C1CCCN(c2nc(OCC34CCCN3CCC4)nc3c(F)c(-c4cccc5cccc(F)c45)ncc23)CC1 ; O=C(Oc1ccc(C(F)(F)F)cc1)C1CCCN(c2nc(OCC34CCCN3CCC4)nc3c(F)c(-c4cccc5cccc(F)c45)ncc23)CC1 ; O=C(Oc1ccccc1)C1CCCN(c2nc(OCC34CCCN3CCC4)nc3c(F)c(-c4cccc5cccc(F)c45)ncc23)C1] 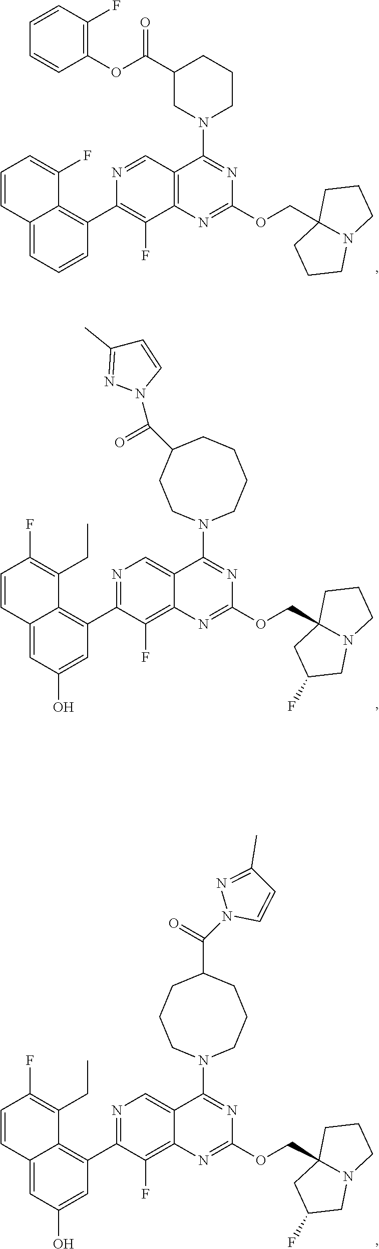 [SMILES: O=C(Oc1ccccc1F)C1CCCN(c2nc(OCC34CCCN3CCC4)nc3c(F)c(-c4cccc5cccc(F)c45)ncc23)C1 ; CCc1c(F)ccc2cc(O)cc(-c3ncc4c(N5CCCCC(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CCCC(C(=O)n6ccc(C)n6)CCC5)nc(OC[C@@]56CCCN5C[C@H](F)C6)nc4c3F)c12] 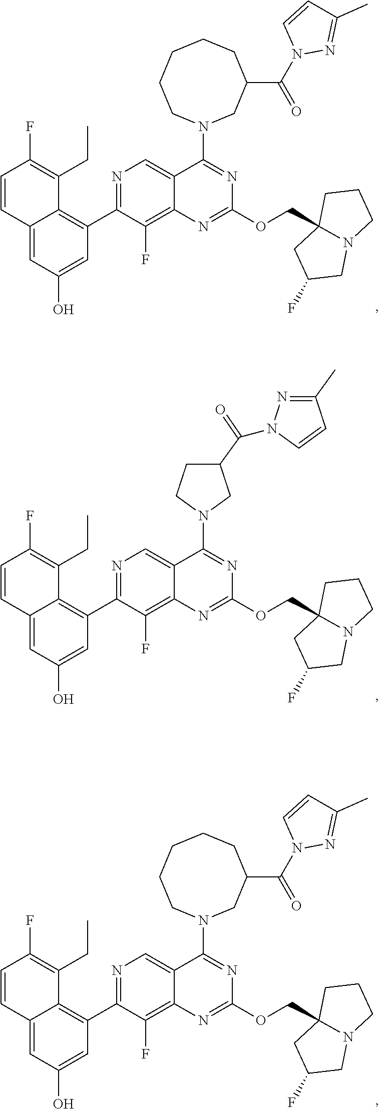 [SMILES: CCc1c(F)ccc2cc(O)cc(-c3ncc4c(N5CCCCCC(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5CCC(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5CCCCCC(C(=O)n6ccc(C)n6)C5)nc(OC[C@@]56CCCN5C[C@H](F)C6)nc4c3F)c12] 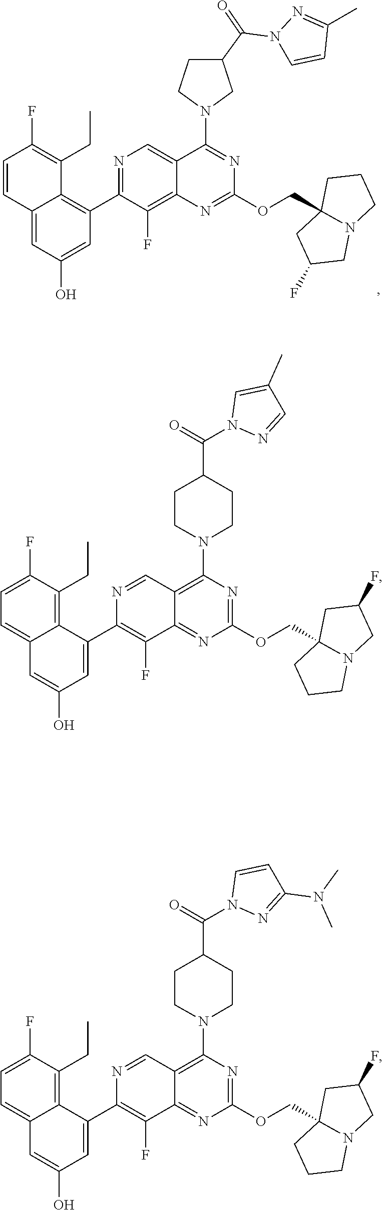 [SMILES: CCc1c(F)ccc2cc(O)cc(-c3ncc4c(N5CCC(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5CCC(C(=O)n6cc(C)cn6)CC5)nc(OC[C@@]56CCCN5C[C@H](F)C6)nc4c3F)c12 ; CCc1c(F)ccc2cc(O)cc(-c3ncc4c(N5CCC(C(=O)n6ccc(N(C)C)n6)CC5)nc(OC[C@@]56CCCN5C[C@H](F)C6)nc4c3F)c12] 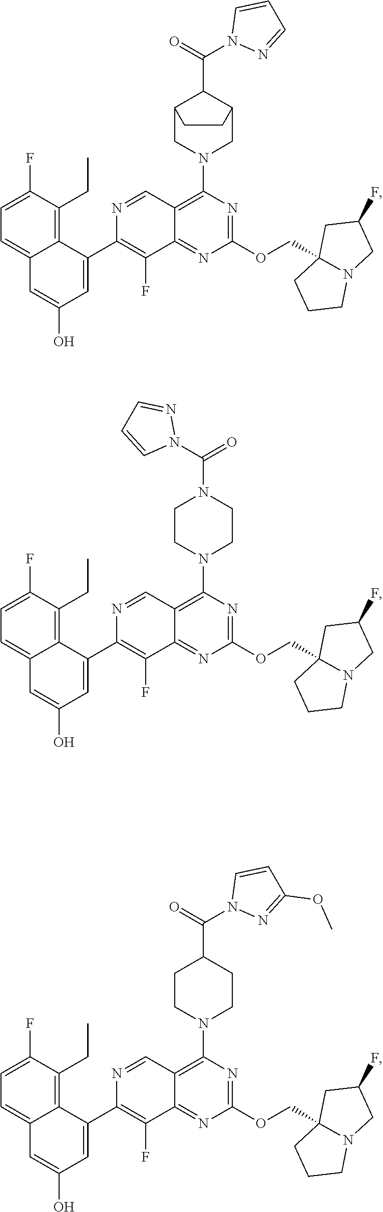 [SMILES: CCc1c(F)ccc2cc(O)cc(-c3ncc4c(N5CC6CCC(C5)C6C(=O)n5cccn5)nc(OC[C@@]56CCCN5C[C@H](F)C6)nc4c3F)c12 ; CCc1c(F)ccc2cc(O)cc(-c3ncc4c(N5CCN(C(=O)n6cccn6)CC5)nc(OC[C@@]56CCCN5C[C@H](F)C6)nc4c3F)c12 ; CCc1c(F)ccc2cc(O)cc(-c3ncc4c(N5CCC(C(=O)n6ccc(OC)n6)CC5)nc(OC[C@@]56CCCN5C[C@H](F)C6)nc4c3F)c12] 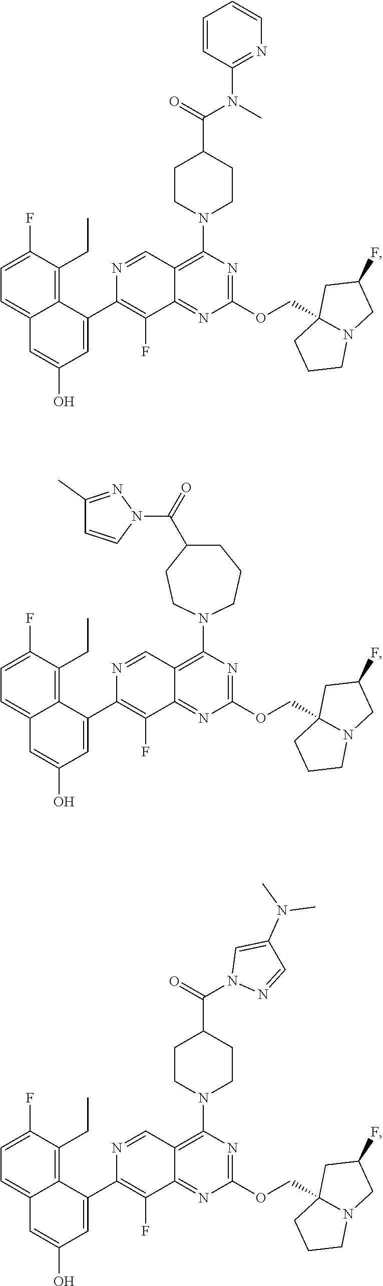 [SMILES: CCc1c(F)ccc2cc(O)cc(-c3ncc4c(N5CCC(C(=O)N(C)c6ccccn6)CC5)nc(OC[C@@]56CCCN5C[C@H](F)C6)nc4c3F)c12 ; CCc1c(F)ccc2cc(O)cc(-c3ncc4c(N5CCCC(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CCC(C(=O)n6cc(N(C)C)cn6)CC5)nc(OC[C@@]56CCCN5C[C@H](F)C6)nc4c3F)c12] 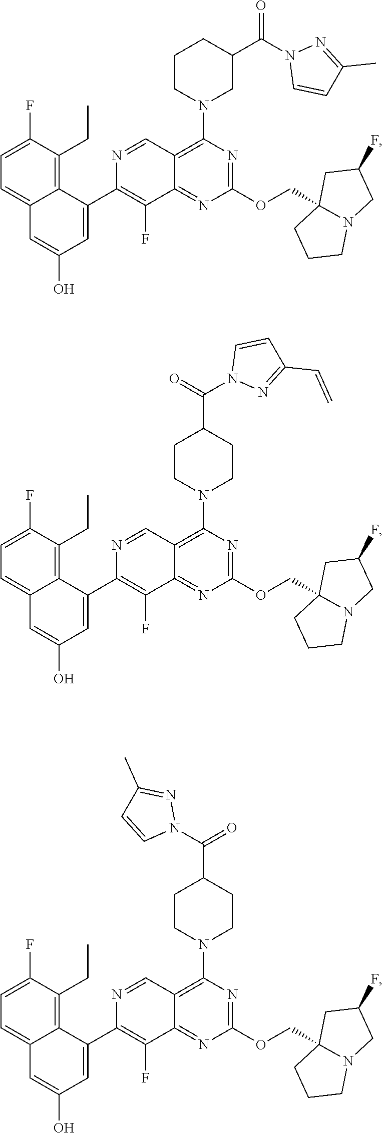 [SMILES: CCc1c(F)ccc2cc(O)cc(-c3ncc4c(N5CCCC(C(=O)n6ccc(C)n6)C5)nc(OC[C@@]56CCCN5C[C@H](F)C6)nc4c3F)c12 ; C=Cc1ccn(C(=O)C2CCN(c3nc(OC[C@@]45CCCN4C[C@H](F)C5)nc4c(F)c(-c5cc(O)cc6ccc(F)c(CC)c56)ncc34)CC2)n1 ; CCc1c(F)ccc2cc(O)cc(-c3ncc4c(N5CCC(C(=O)n6ccc(C)n6)CC5)nc(OC[C@@]56CCCN5C[C@H](F)C6)nc4c3F)c12] 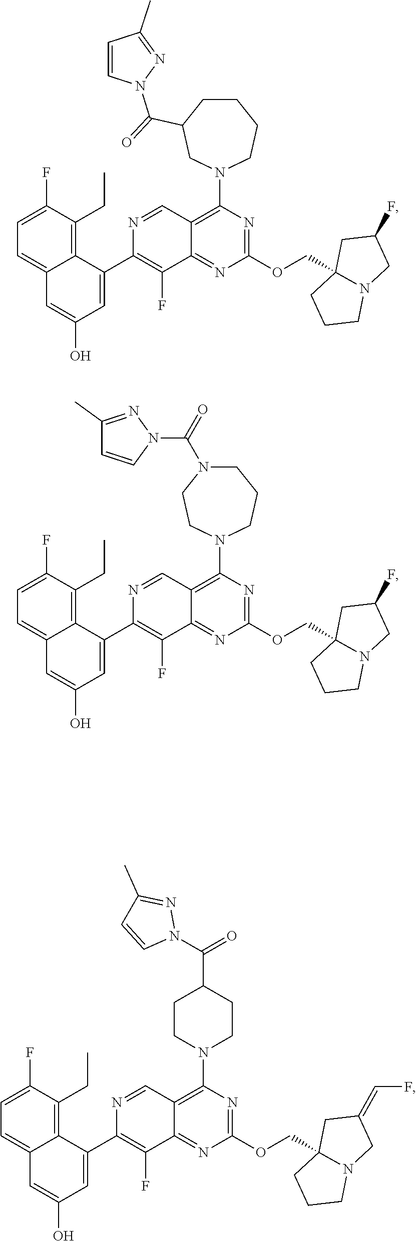 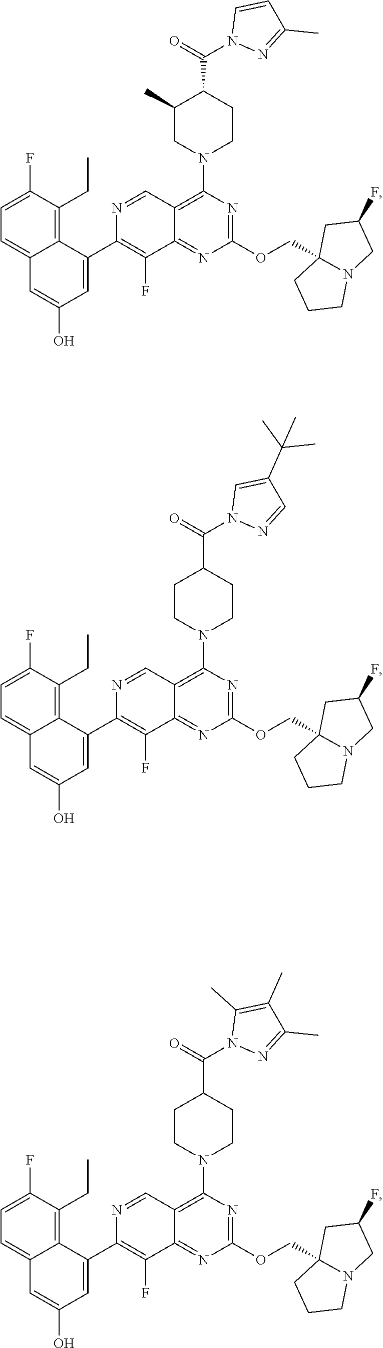 [SMILES: CCc1c(F)ccc2cc(O)cc(-c3ncc4c(N5CC[C@@H](C(=O)n6ccc(C)n6)[C@H](C)C5)nc(OC[C@@]56CCCN5C[C@H](F)C6)nc4c3F)c12 ; CCc1c(F)ccc2cc(O)cc(-c3ncc4c(N5CCC(C(=O)n6cc(C(C)(C)C)cn6)CC5)nc(OC[C@@]56CCCN5C[C@H](F)C6)nc4c3F)c12 ; CCc1c(F)ccc2cc(O)cc(-c3ncc4c(N5CCC(C(=O)n6nc(C)c(C)c6C)CC5)nc(OC[C@@]56CCCN5C[C@H](F)C6)nc4c3F)c12] 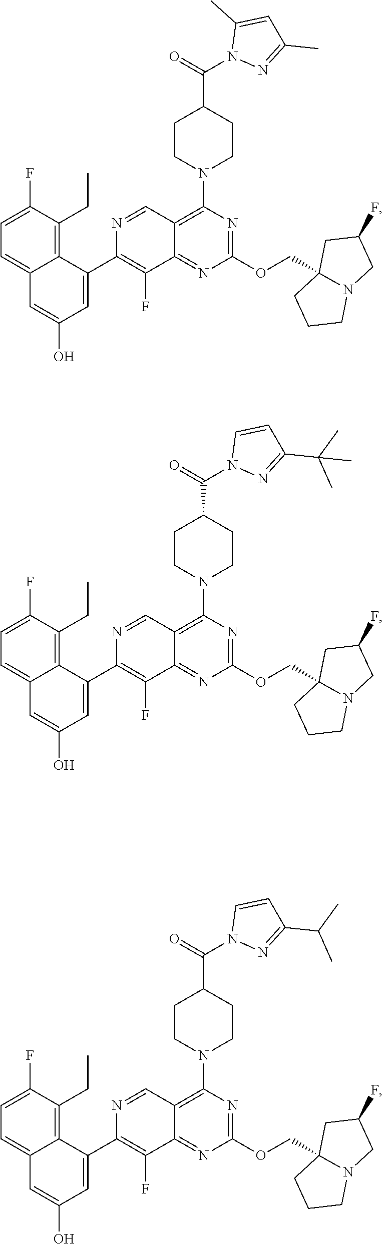 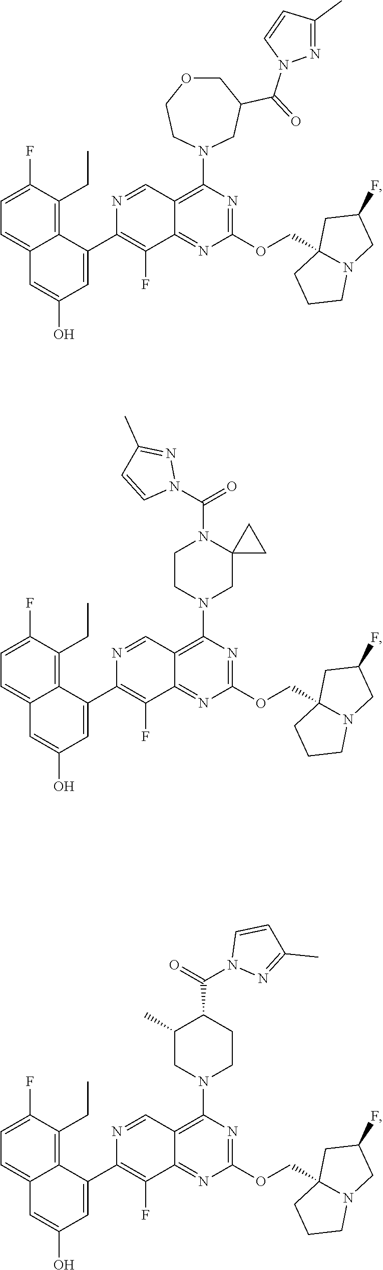 [SMILES: CCc1c(F)ccc2cc(O)cc(-c3ncc4c(N5CCOCC(C(=O)n6ccc(C)n6)C5)nc(OC[C@@]56CCCN5C[C@H](F)C6)nc4c3F)c12 ; CCc1c(F)ccc2cc(O)cc(-c3ncc4c(N5CCN(C(=O)n6ccc(C)n6)C6(CC6)C5)nc(OC[C@@]56CCCN5C[C@H](F)C6)nc4c3F)c12 ; CCc1c(F)ccc2cc(O)cc(-c3ncc4c(N5CC[C@@H](C(=O)n6ccc(C)n6)[C@@H](C)C5)nc(OC[C@@]56CCCN5C[C@H](F)C6)nc4c3F)c12] 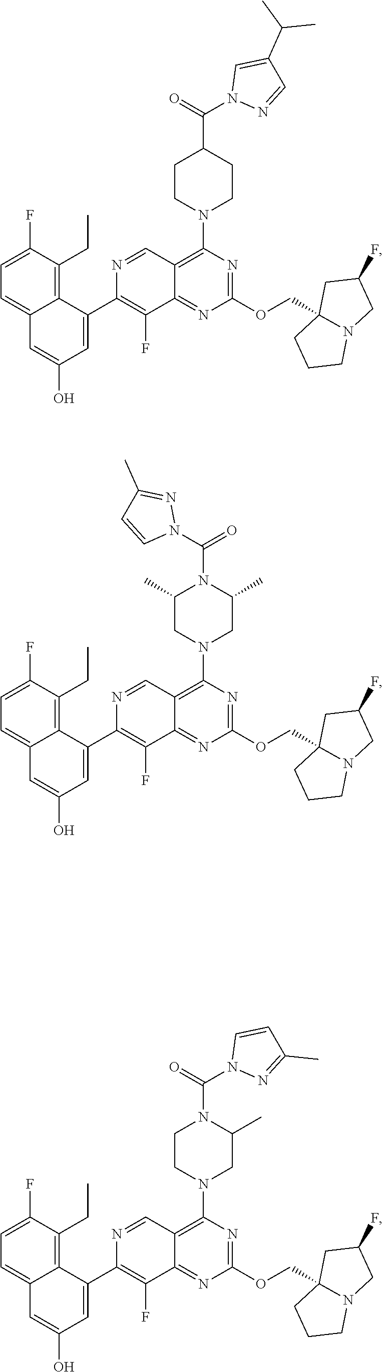 [SMILES: CCc1c(F)ccc2cc(O)cc(-c3ncc4c(N5CCC(C(=O)n6cc(C(C)C)cn6)CC5)nc(OC[C@@]56CCCN5C[C@H](F)C6)nc4c3F)c12 ; CCc1c(F)ccc2cc(O)cc(-c3ncc4c(N5C[C@@H](C)N(C(=O)n6ccc(C)n6)[C@@H](C)C5)nc(OC[C@@]56CCCN5C[C@H](F)C6)nc4c3F)c12 ; CCc1c(F)ccc2cc(O)cc(-c3ncc4c(N5CCN(C(=O)n6ccc(C)n6)C(C)C5)nc(OC[C@@]56CCCN5C[C@H](F)C6)nc4c3F)c12] 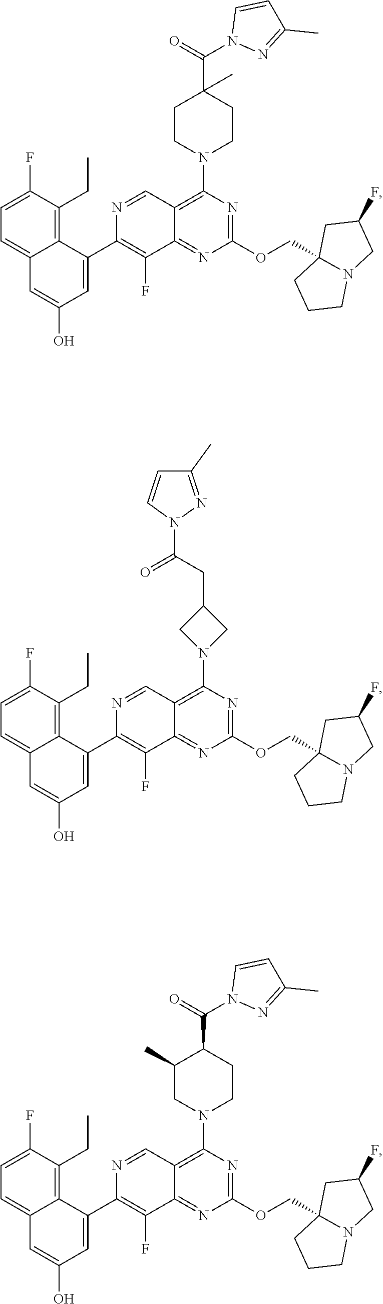 [SMILES: CCc1c(F)ccc2cc(O)cc(-c3ncc4c(N5CCC(C)(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CC(CC(=O)n6ccc(C)n6)C5)nc(OC[C@@]56CCCN5C[C@H](F)C6)nc4c3F)c12 ; CCc1c(F)ccc2cc(O)cc(-c3ncc4c(N5CC[C@H](C(=O)n6ccc(C)n6)[C@H](C)C5)nc(OC[C@@]56CCCN5C[C@H](F)C6)nc4c3F)c12] 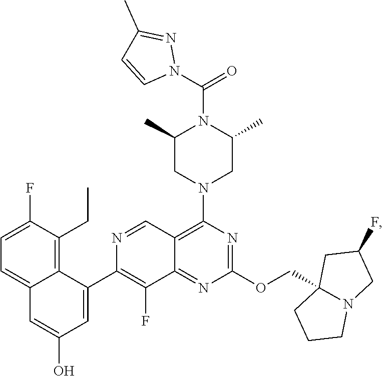 [SMILES: CCc1c(F)ccc2cc(O)cc(-c3ncc4c(N5C[C@@H](C)N(C(=O)n6ccc(C)n6)[C@H](C)C5)nc(OC[C@@]56CCCN5C[C@H](F)C6)nc4c3F)c12] 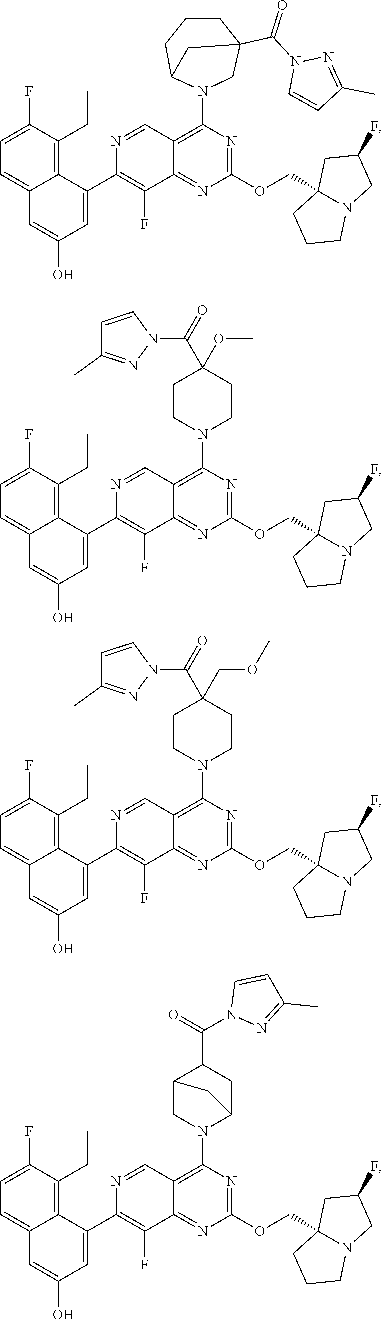 [SMILES: CCc1c(F)ccc2cc(O)cc(-c3ncc4c(N5CC6(C(=O)n7ccc(C)n7)CCCC5C6)nc(OC[C@@]56CCCN5C[C@H](F)C6)nc4c3F)c12 ; CCc1c(F)ccc2cc(O)cc(-c3ncc4c(N5CCC(OC)(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CCC(COC)(C(=O)n6ccc(C)n6)CC5)nc(OC[C@@]56CCCN5C[C@H](F)C6)nc4c3F)c12 ; CCc1c(F)ccc2cc(O)cc(-c3ncc4c(N5CC6CC5CC6C(=O)n5ccc(C)n5)nc(OC[C@@]56CCCN5C[C@H](F)C6)nc4c3F)c12] 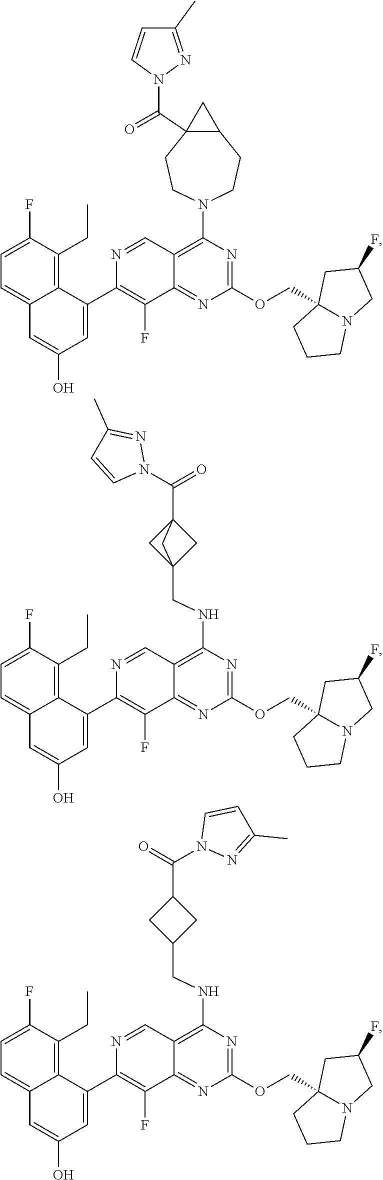 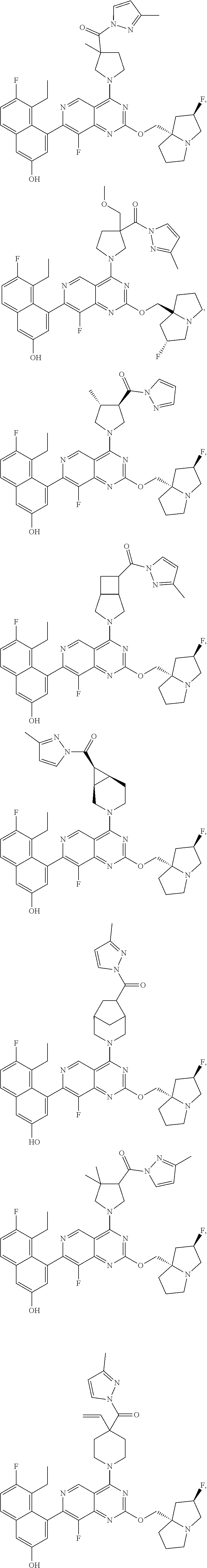 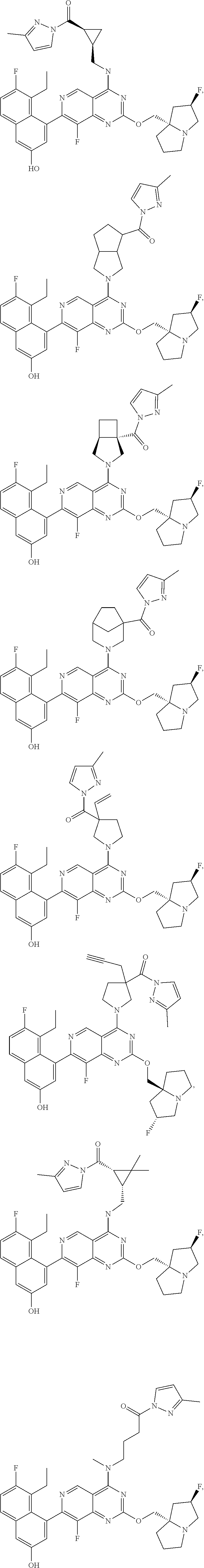 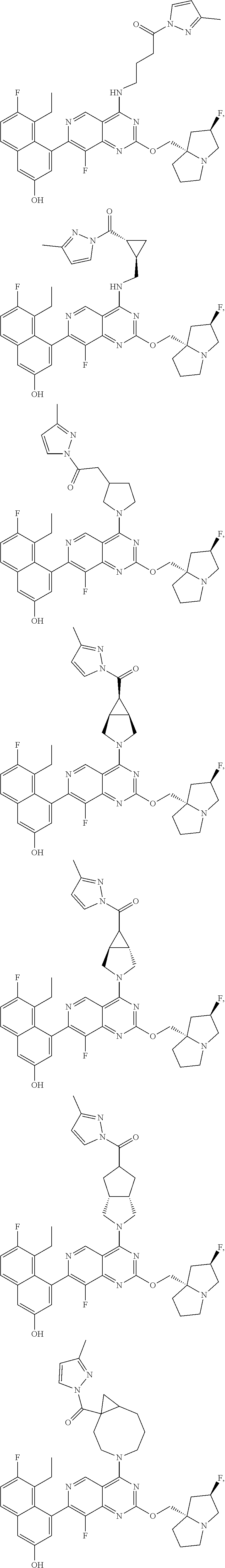 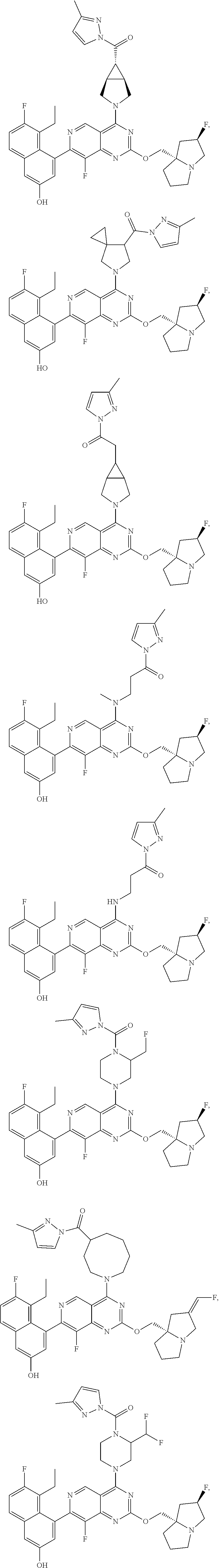 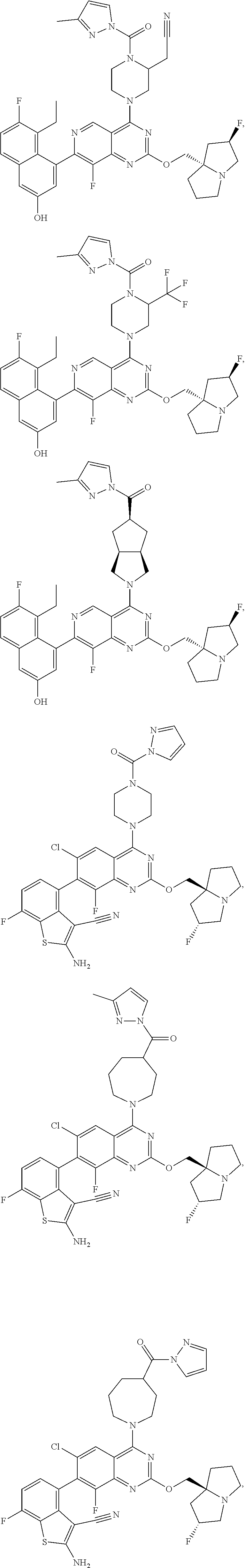 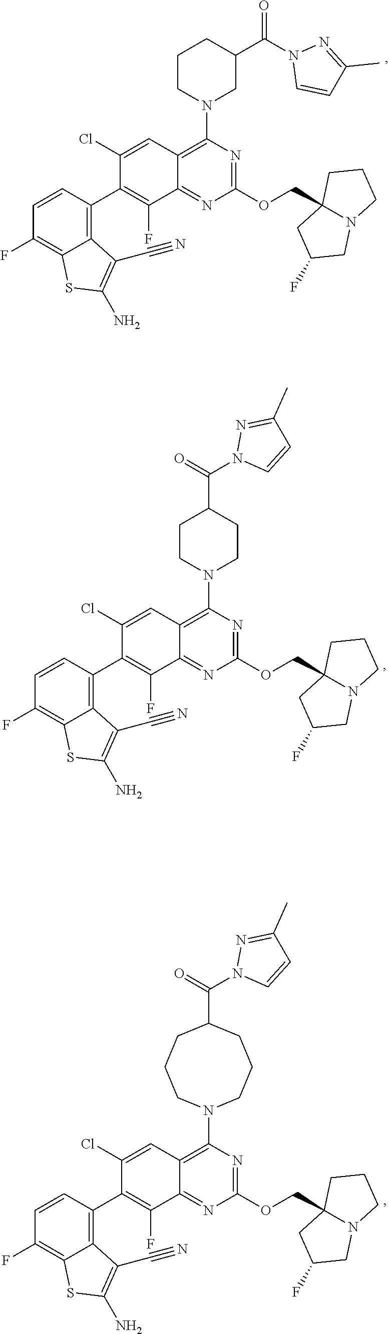 [SMILES: Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)C2)n1 ; Cc1ccn(C(=O)C2CCN(c3nc(OC[C@@]45CCCN4C[C@H](F)C5)nc4c(F)c(-c5ccc(F)c6sc(N)c(C#N)c56)c(Cl)cc34)CC2)n1 ; Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CCC2)n1] 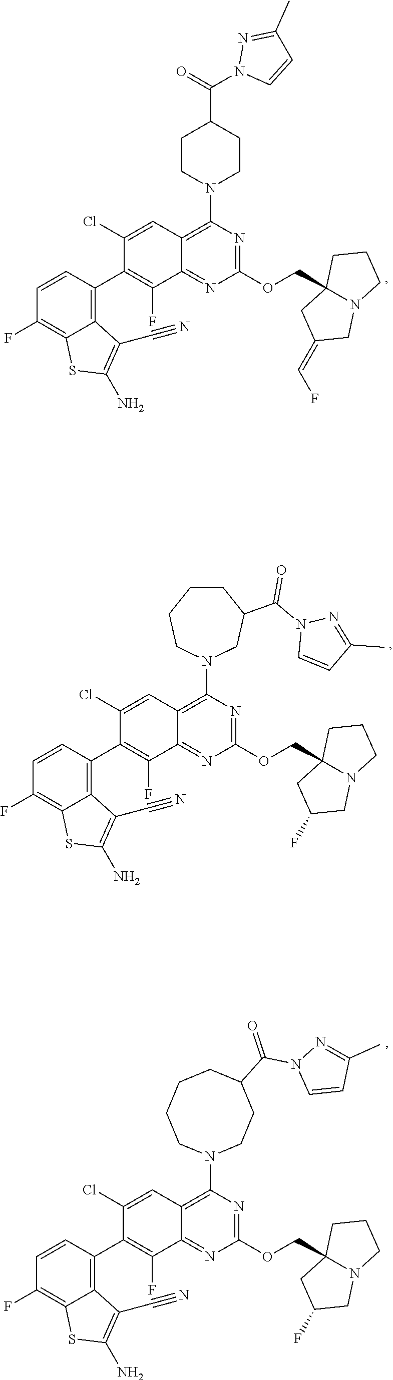 [SMILES: Cc1ccn(C(=O)C2CCN(c3nc(OC[C@@]45CCCN4C/C(=C\F)C5)nc4c(F)c(-c5ccc(F)c6sc(N)c(C#N)c56)c(Cl)cc34)CC2)n1 ; Cc1ccn(C(=O)C2CCCCN(c3nc(OC[C@@]45CCCN4C[C@H](F)C5)nc4c(F)c(-c5ccc(F)c6sc(N)c(C#N)c56)c(Cl)cc34)C2)n1 ; Cc1ccn(C(=O)C2CCCCN(c3nc(OC[C@@]45CCCN4C[C@H](F)C5)nc4c(F)c(-c5ccc(F)c6sc(N)c(C#N)c56)c(Cl)cc34)CC2)n1] 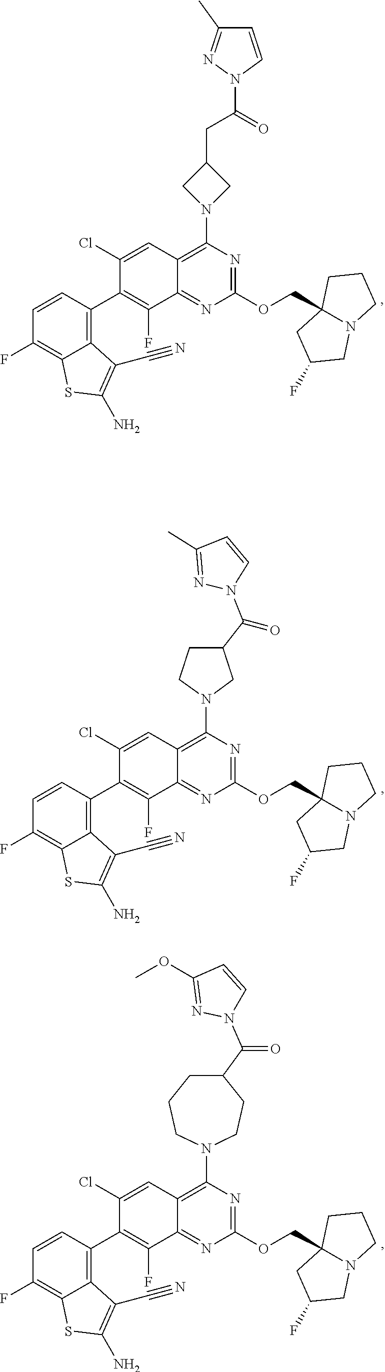 [SMILES: Cc1ccn(C(=O)CC2CN(c3nc(OC[C@@]45CCCN4C[C@H](F)C5)nc4c(F)c(-c5ccc(F)c6sc(N)c(C#N)c56)c(Cl)cc34)C2)n1 ; Cc1ccn(C(=O)C2CCN(c3nc(OC[C@@]45CCCN4C[C@H](F)C5)nc4c(F)c(-c5ccc(F)c6sc(N)c(C#N)c56)c(Cl)cc34)C2)n1 ; COc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1] 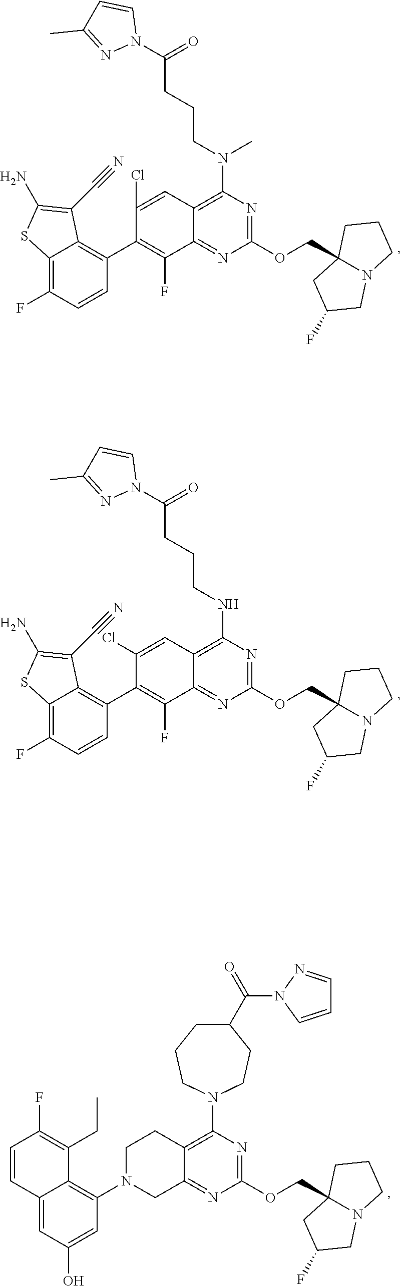 [SMILES: Cc1ccn(C(=O)CCCN(C)c2nc(OC[C@@]34CCCN3C[C@H](F)C4)nc3c(F)c(-c4ccc(F)c5sc(N)c(C#N)c45)c(Cl)cc23)n1 ; Cc1ccn(C(=O)CCCNc2nc(OC[C@@]34CCCN3C[C@H](F)C4)nc3c(F)c(-c4ccc(F)c5sc(N)c(C#N)c45)c(Cl)cc23)n1 ; CCc1c(F)ccc2cc(O)cc(N3CCc4c(nc(OC[C@@]56CCCN5C[C@H](F)C6)nc4N4CCCC(C(=O)n5cccn5)CC4)C3)c12] 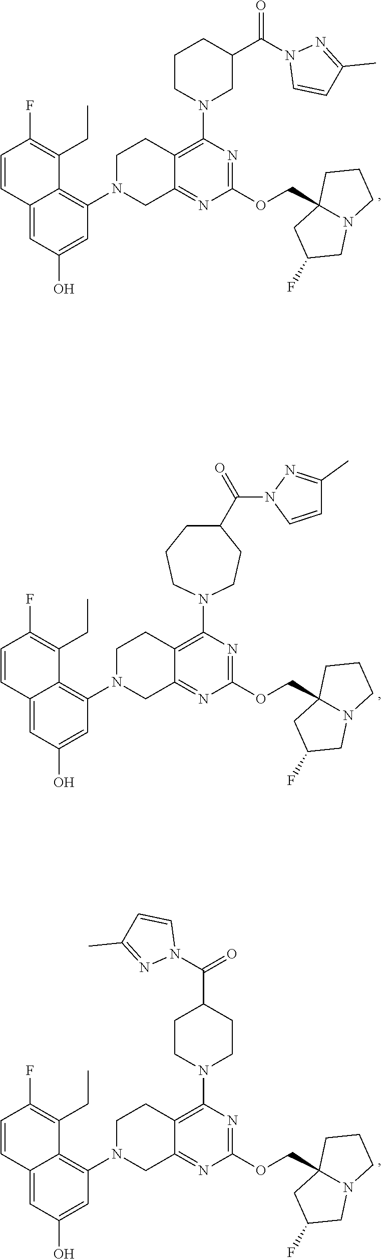 [SMILES: CCc1c(F)ccc2cc(O)cc(N3CCc4c(nc(OC[C@@]56CCCN5C[C@H](F)C6)nc4N4CCCC(C(=O)n5ccc(C)n5)C4)C3)c12 ; CCc1c(F)ccc2cc(O)cc(N3CCc4c(nc(OC[C@@]56CCCN5C[C@H](F)C6)nc4N4CCCC(C(=O)n5ccc(C)n5)CC4)C3)c12 ; CCc1c(F)ccc2cc(O)cc(N3CCc4c(nc(OC[C@@]56CCCN5C[C@H](F)C6)nc4N4CCC(C(=O)n5ccc(C)n5)CC4)C3)c12] 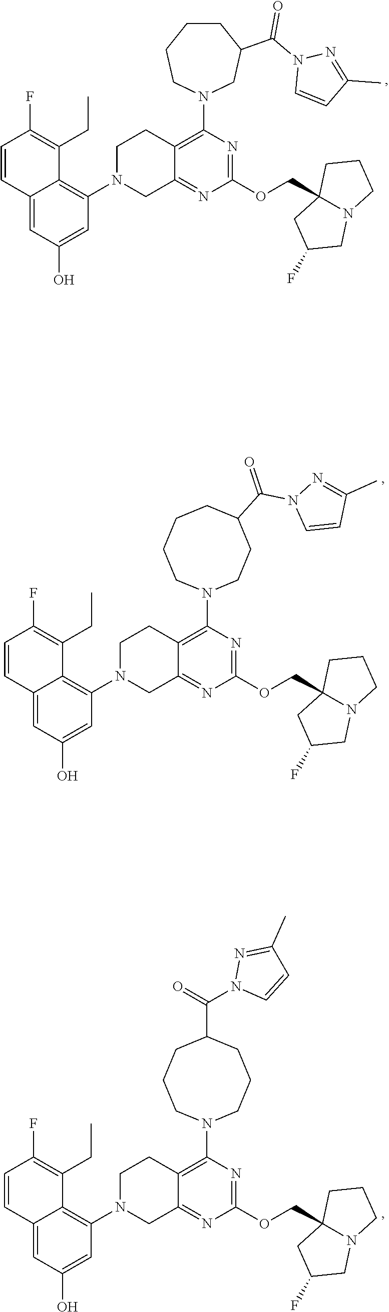 [SMILES: CCc1c(F)ccc2cc(O)cc(N3CCc4c(nc(OC[C@@]56CCCN5C[C@H](F)C6)nc4N4CCCCC(C(=O)n5ccc(C)n5)C4)C3)c12 ; CCc1c(F)ccc2cc(O)cc(N3CCc4c(nc(OC[C@@]56CCCN5C[C@H](F)C6)nc4N4CCCCC(C(=O)n5ccc(C)n5)CC4)C3)c12 ; CCc1c(F)ccc2cc(O)cc(N3CCc4c(nc(OC[C@@]56CCCN5C[C@H](F)C6)nc4N4CCCC(C(=O)n5ccc(C)n5)CCC4)C3)c12] 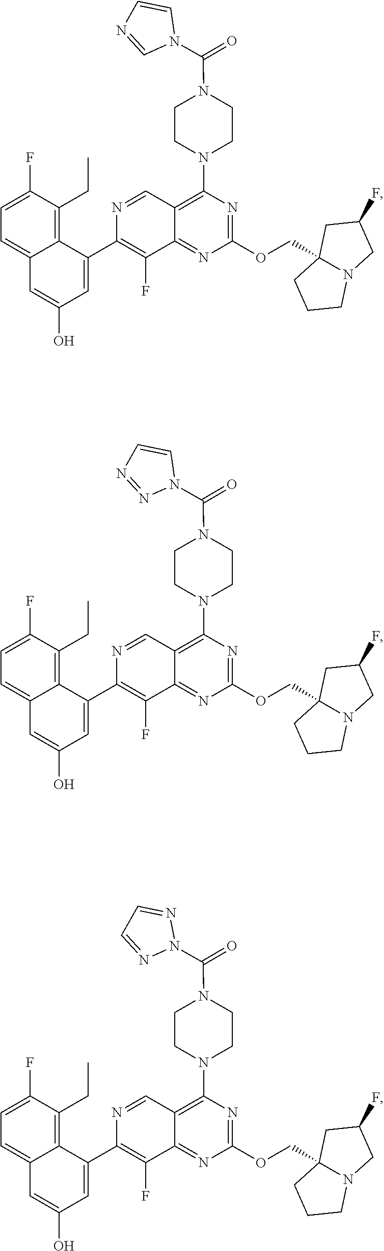 [SMILES: CCc1c(F)ccc2cc(O)cc(-c3ncc4c(N5CCN(C(=O)n6ccnc6)CC5)nc(OC[C@@]56CCCN5C[C@H](F)C6)nc4c3F)c12 ; CCc1c(F)ccc2cc(O)cc(-c3ncc4c(N5CCN(C(=O)n6ccnn6)CC5)nc(OC[C@@]56CCCN5C[C@H](F)C6)nc4c3F)c12 ; CCc1c(F)ccc2cc(O)cc(-c3ncc4c(N5CCN(C(=O)n6nccn6)CC5)nc(OC[C@@]56CCCN5C[C@H](F)C6)nc4c3F)c12] 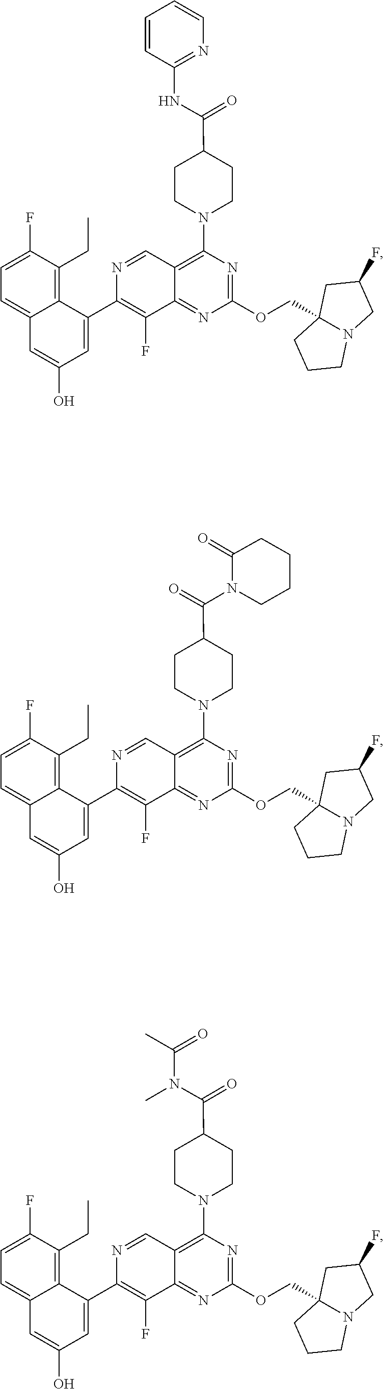 [SMILES: CCc1c(F)ccc2cc(O)cc(-c3ncc4c(N5CCC(C(=O)Nc6ccccn6)CC5)nc(OC[C@@]56CCCN5C[C@H](F)C6)nc4c3F)c12 ; CCc1c(F)ccc2cc(O)cc(-c3ncc4c(N5CCC(C(=O)N6CCCCC6=O)CC5)nc(OC[C@@]56CCCN5C[C@H](F)C6)nc4c3F)c12 ; CCc1c(F)ccc2cc(O)cc(-c3ncc4c(N5CCC(C(=O)N(C)C(C)=O)CC5)nc(OC[C@@]56CCCN5C[C@H](F)C6)nc4c3F)c12] 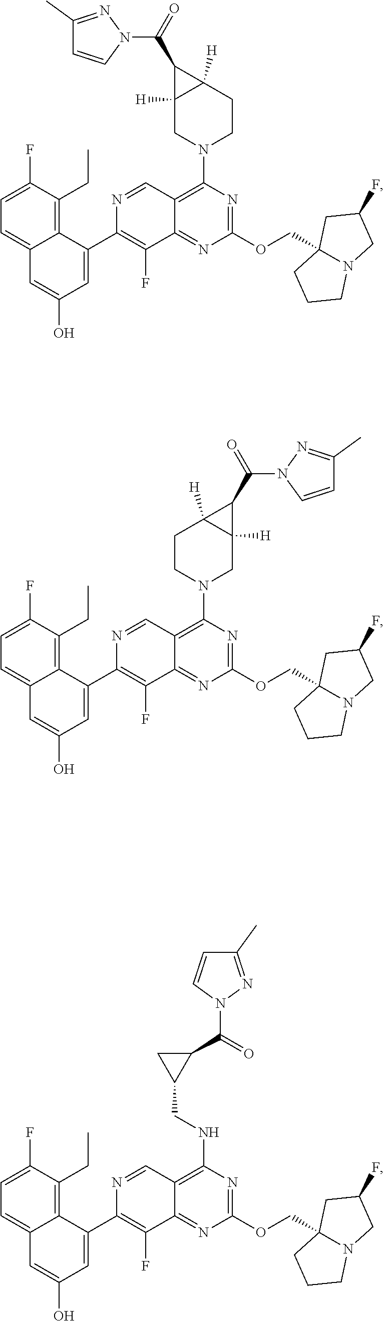 [SMILES: CCc1c(F)ccc2cc(O)cc(-c3ncc4c(N5CC[C@H]6[C@@H](C5)[C@H]6C(=O)n5ccc(C)n5)nc(OC[C@@]56CCCN5C[C@H](F)C6)nc4c3F)c12 ; CCc1c(F)ccc2cc(O)cc(-c3ncc4c(N5CC[C@@H]6[C@H](C5)[C@@H]6C(=O)n5ccc(C)n5)nc(OC[C@@]56CCCN5C[C@H](F)C6)nc4c3F)c12 ; CCc1c(F)ccc2cc(O)cc(-c3ncc4c(NC[C@@H]5C[C@H]5C(=O)n5ccc(C)n5)nc(OC[C@@]56CCCN5C[C@H](F)C6)nc4c3F)c12] 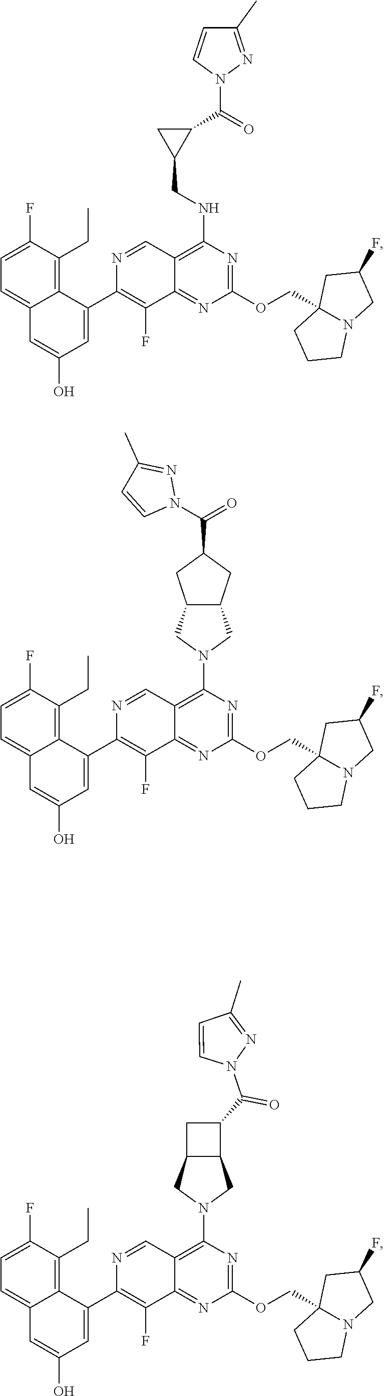 [SMILES: CCc1c(F)ccc2cc(O)cc(-c3ncc4c(NC[C@H]5C[C@@H]5C(=O)n5ccc(C)n5)nc(OC[C@@]56CCCN5C[C@H](F)C6)nc4c3F)c12 ; CCc1c(F)ccc2cc(O)cc(-c3ncc4c(N5C[C@H]6C[C@H](C(=O)n7ccc(C)n7)C[C@H]6C5)nc(OC[C@@]56CCCN5C[C@H](F)C6)nc4c3F)c12 ; CCc1c(F)ccc2cc(O)cc(-c3ncc4c(N5C[C@H]6C[C@H](C(=O)n7ccc(C)n7)[C@H]6C5)nc(OC[C@@]56CCCN5C[C@H](F)C6)nc4c3F)c12] 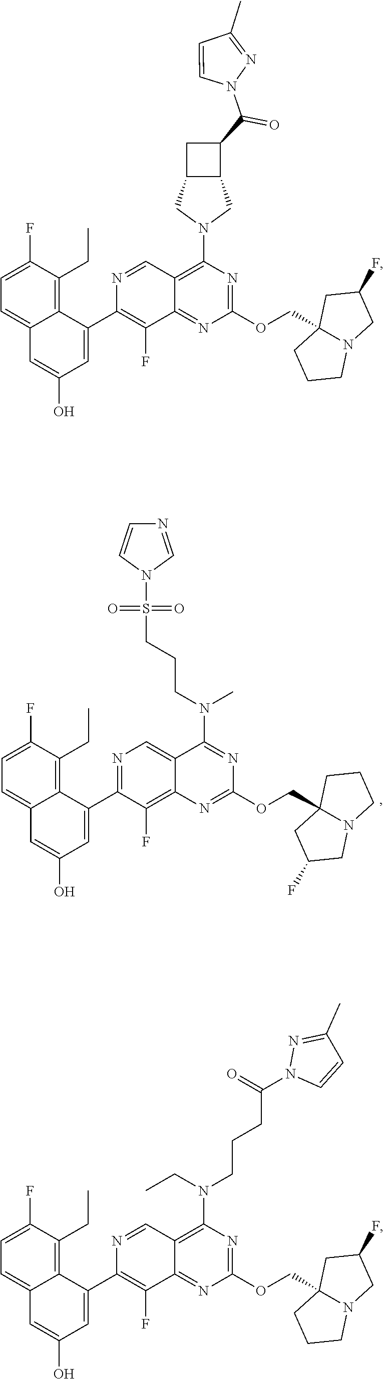 [SMILES: CCc1c(F)ccc2cc(O)cc(-c3ncc4c(N5C[C@@H]6C[C@@H](C(=O)n7ccc(C)n7)[C@@H]6C5)nc(OC[C@@]56CCCN5C[C@H](F)C6)nc4c3F)c12 ; CCc1c(F)ccc2cc(O)cc(-c3ncc4c(N(C)CCCS(=O)(=O)n5ccnc5)nc(OC[C@@]56CCCN5C[C@H](F)C6)nc4c3F)c12 ; CCc1c(F)ccc2cc(O)cc(-c3ncc4c(N(CC)CCCC(=O)n5ccc(C)n5)nc(OC[C@@]56CCCN5C[C@H](F)C6)nc4c3F)c12] 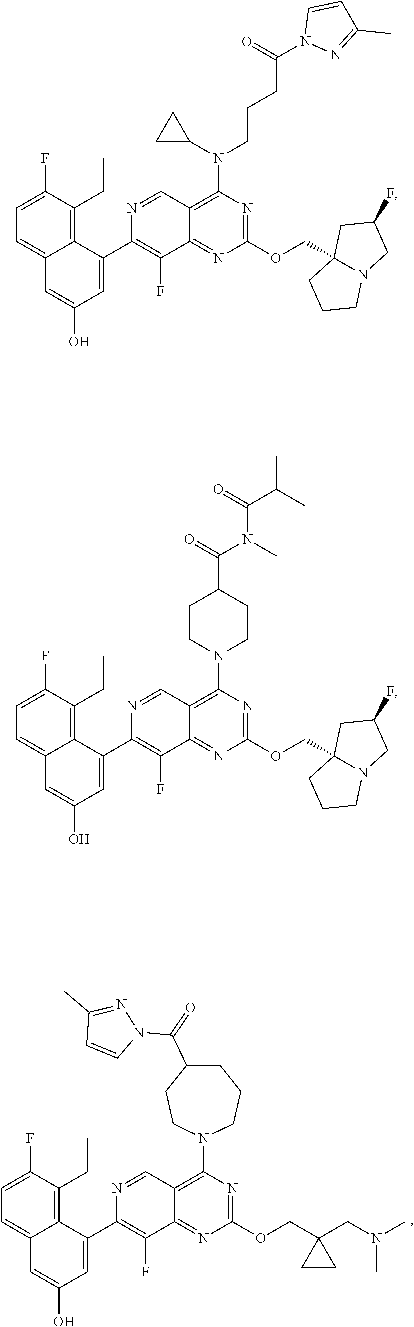 [SMILES: CCc1c(F)ccc2cc(O)cc(-c3ncc4c(N(CCCC(=O)n5ccc(C)n5)C5CC5)nc(OC[C@@]56CCCN5C[C@H](F)C6)nc4c3F)c12 ; CCc1c(F)ccc2cc(O)cc(-c3ncc4c(N5CCC(C(=O)N(C)C(=O)C(C)C)CC5)nc(OC[C@@]56CCCN5C[C@H](F)C6)nc4c3F)c12 ; CCc1c(F)ccc2cc(O)cc(-c3ncc4c(N5CCCC(C(=O)n6ccc(C)n6)CC5)nc(OCC5(CN(C)C)CC5)nc4c3F)c12] 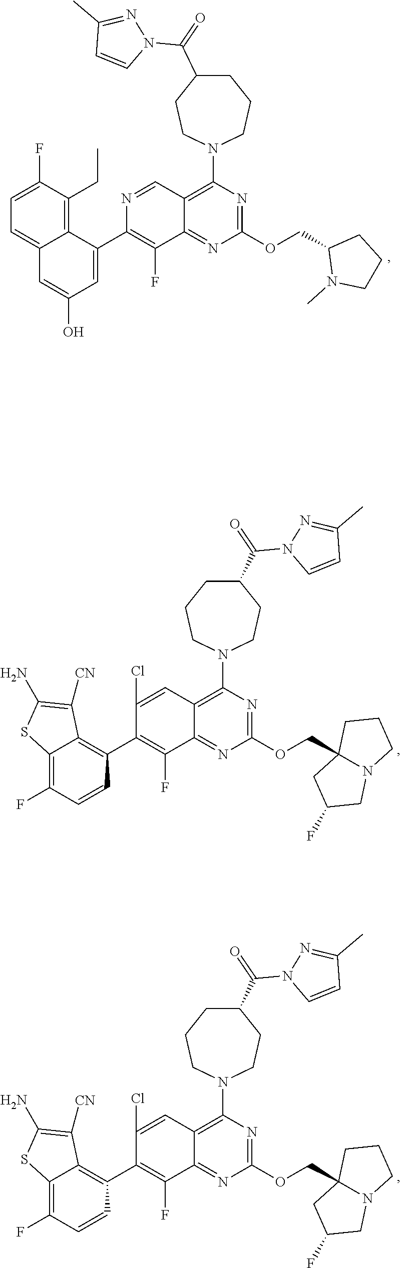 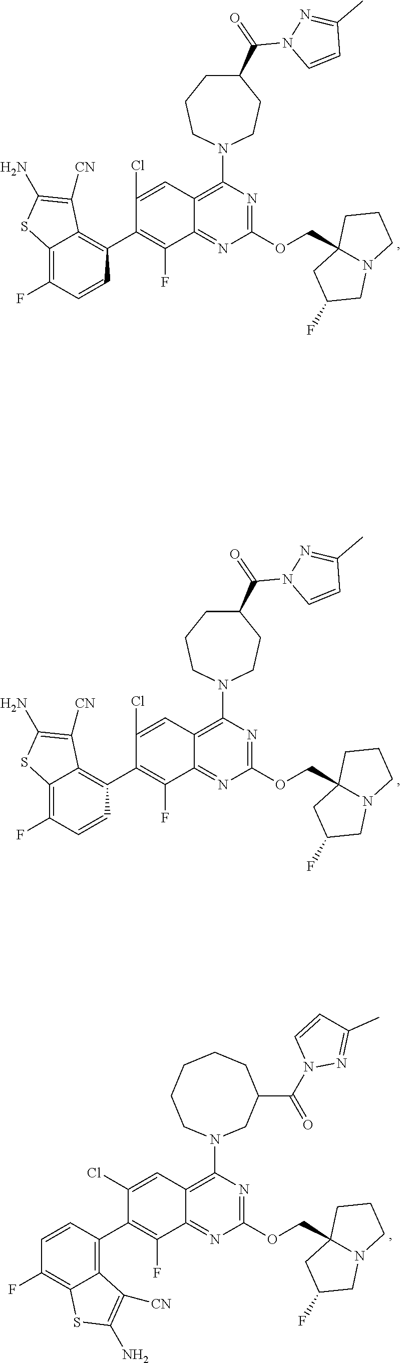 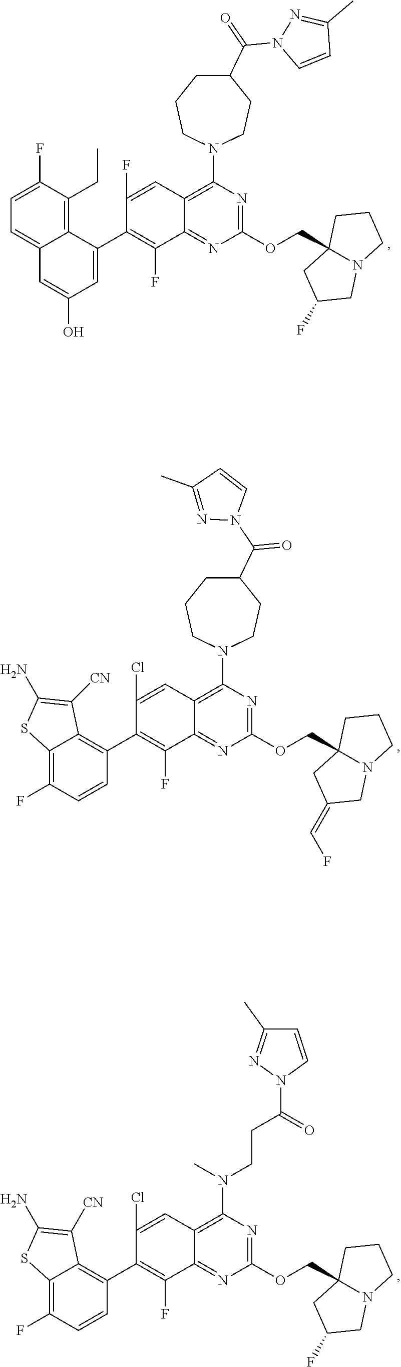 [SMILES: CCc1c(F)ccc2cc(O)cc(-c3c(F)cc4c(N5CCCC(C(=O)n6ccc(C)n6)CC5)nc(OC[C@@]56CCCN5C[C@H](F)C6)nc4c3F)c12 ; Cc1ccn(C(=O)C2CCCN(c3nc(OC[C@@]45CCCN4C/C(=C\F)C5)nc4c(F)c(-c5ccc(F)c6sc(N)c(C#N)c56)c(Cl)cc34)CC2)n1 ; Cc1ccn(C(=O)CCN(C)c2nc(OC[C@@]34CCCN3C[C@H](F)C4)nc3c(F)c(-c4ccc(F)c5sc(N)c(C#N)c45)c(Cl)cc23)n1] 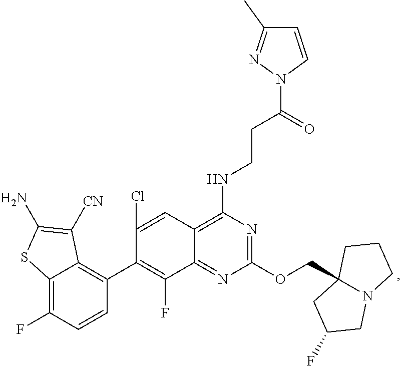 [SMILES: Cc1ccn(C(=O)CCNc2nc(OC[C@@]34CCCN3C[C@H](F)C4)nc3c(F)c(-c4ccc(F)c5sc(N)c(C#N)c45)c(Cl)cc23)n1] 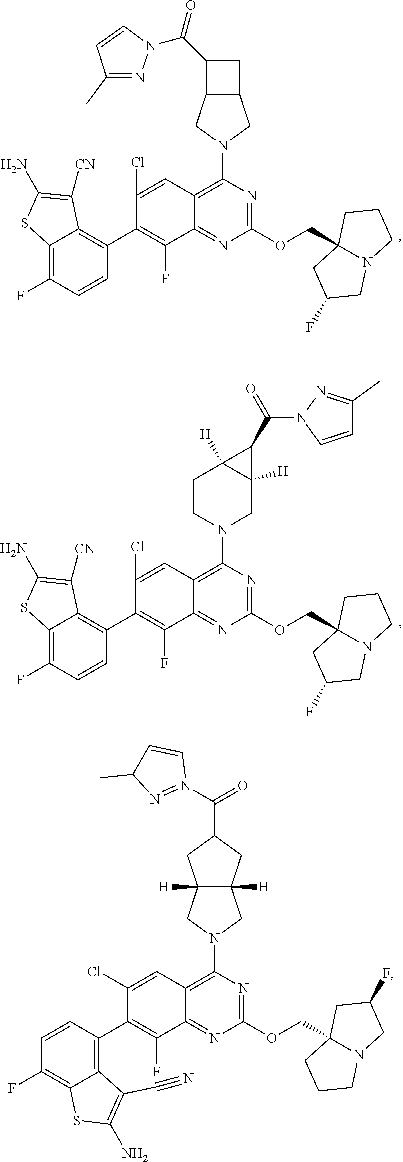 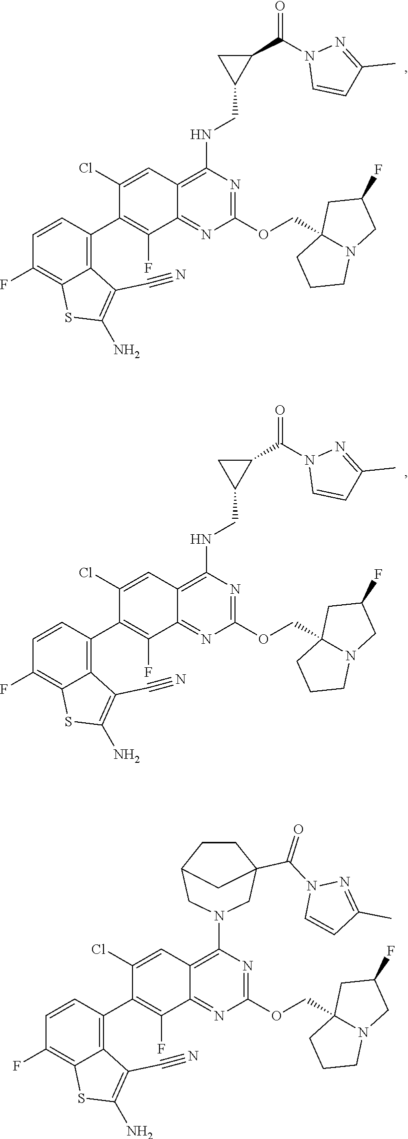 [SMILES: Cc1ccn(C(=O)[C@@H]2C[C@H]2CNc2nc(OC[C@@]34CCCN3C[C@H](F)C4)nc3c(F)c(-c4ccc(F)c5sc(N)c(C#N)c45)c(Cl)cc23)n1 ; Cc1ccn(C(=O)[C@H]2C[C@H]2CNc2nc(OC[C@@]34CCCN3C[C@H](F)C4)nc3c(F)c(-c4ccc(F)c5sc(N)c(C#N)c45)c(Cl)cc23)n1 ; Cc1ccn(C(=O)C23CCC(CN(c4nc(OC[C@@]56CCCN5C[C@H](F)C6)nc5c(F)c(-c6ccc(F)c7sc(N)c(C#N)c67)c(Cl)cc45)C2)C3)n1] 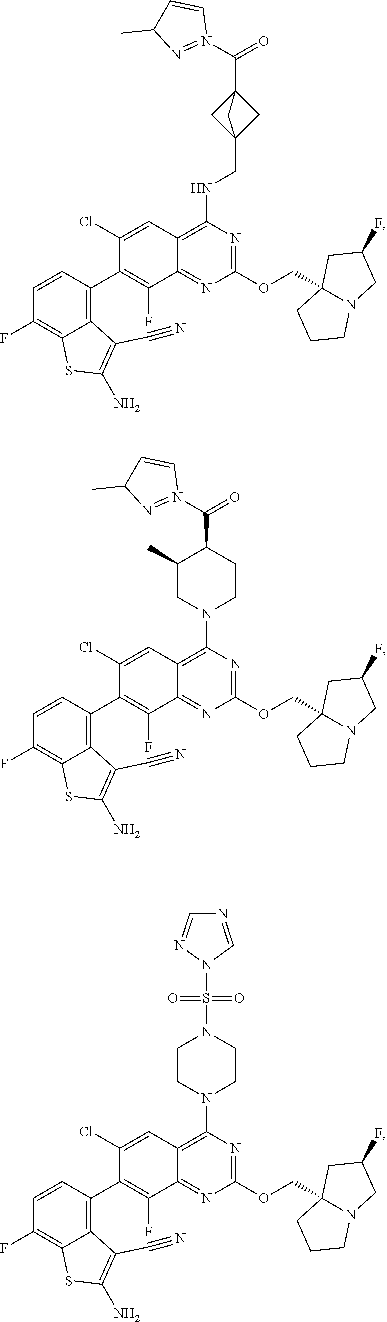 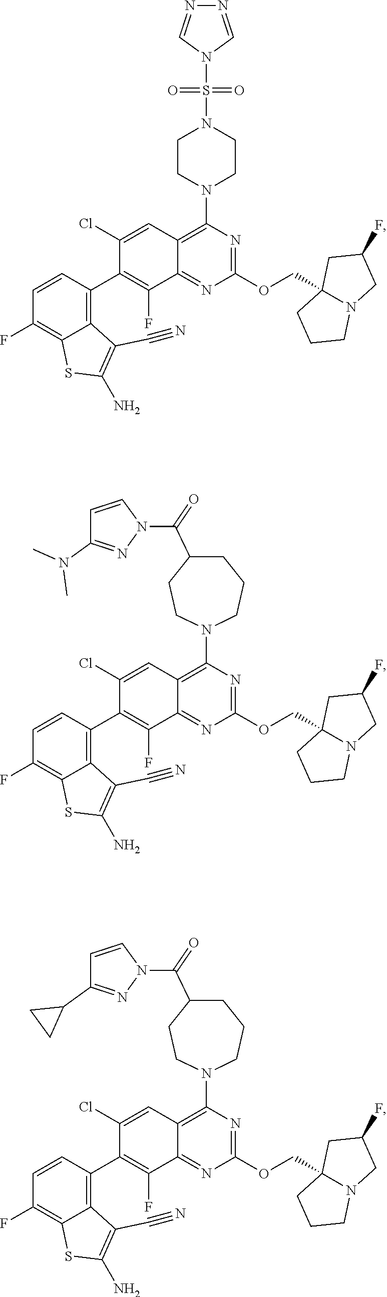 [SMILES: N#Cc1c(N)sc2c(F)ccc(-c3c(Cl)cc4c(N5CCN(S(=O)(=O)n6cnnc6)CC5)nc(OC[C@@]56CCCN5C[C@H](F)C6)nc4c3F)c12 ; CN(C)c1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; N#Cc1c(N)sc2c(F)ccc(-c3c(Cl)cc4c(N5CCCC(C(=O)n6ccc(C7CC7)n6)CC5)nc(OC[C@@]56CCCN5C[C@H](F)C6)nc4c3F)c12] 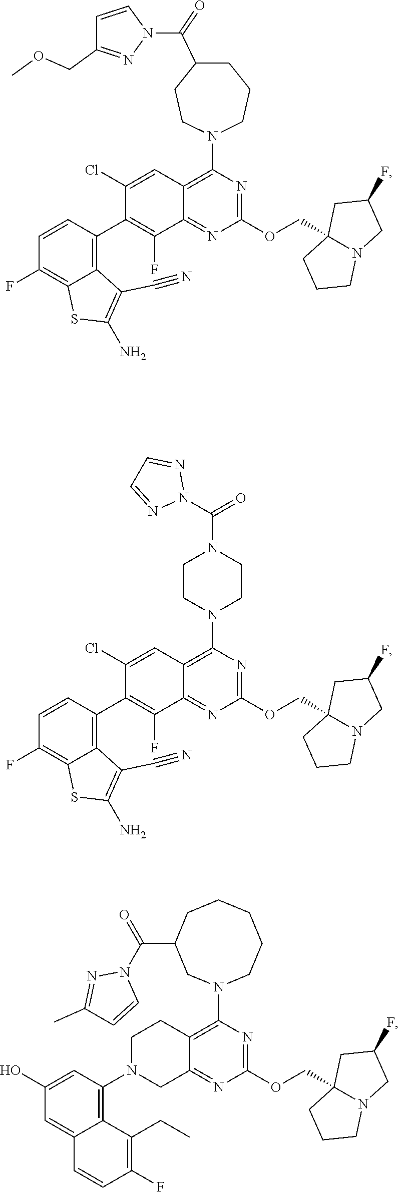 [SMILES: COCc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; N#Cc1c(N)sc2c(F)ccc(-c3c(Cl)cc4c(N5CCN(C(=O)n6nccn6)CC5)nc(OC[C@@]56CCCN5C[C@H](F)C6)nc4c3F)c12 ; CCc1c(F)ccc2cc(O)cc(N3CCc4c(nc(OC[C@@]56CCCN5C[C@H](F)C6)nc4N4CCCCCC(C(=O)n5ccc(C)n5)C4)C3)c12] 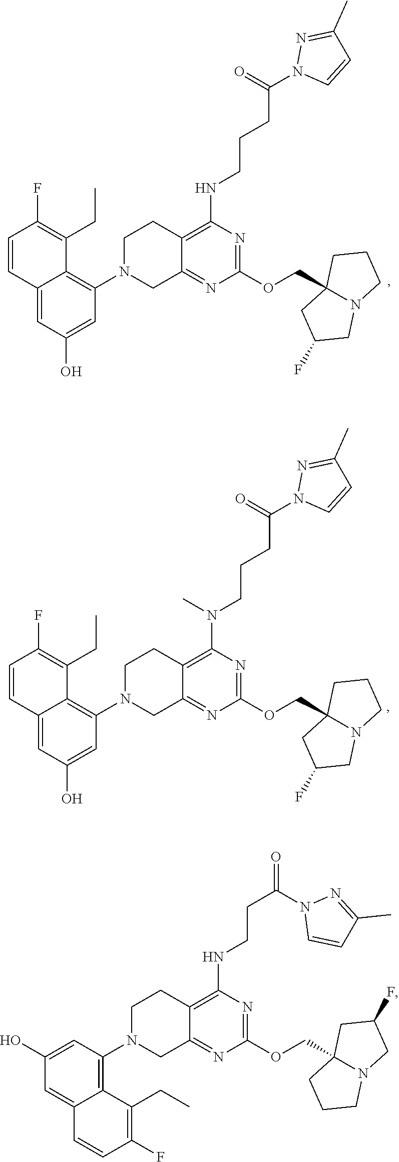 [SMILES: CCc1c(F)ccc2cc(O)cc(N3CCc4c(nc(OC[C@@]56CCCN5C[C@H](F)C6)nc4NCCCC(=O)n4ccc(C)n4)C3)c12 ; CCc1c(F)ccc2cc(O)cc(N3CCc4c(nc(OC[C@@]56CCCN5C[C@H](F)C6)nc4N(C)CCCC(=O)n4ccc(C)n4)C3)c12 ; CCc1c(F)ccc2cc(O)cc(N3CCc4c(nc(OC[C@@]56CCCN5C[C@H](F)C6)nc4NCCC(=O)n4ccc(C)n4)C3)c12] 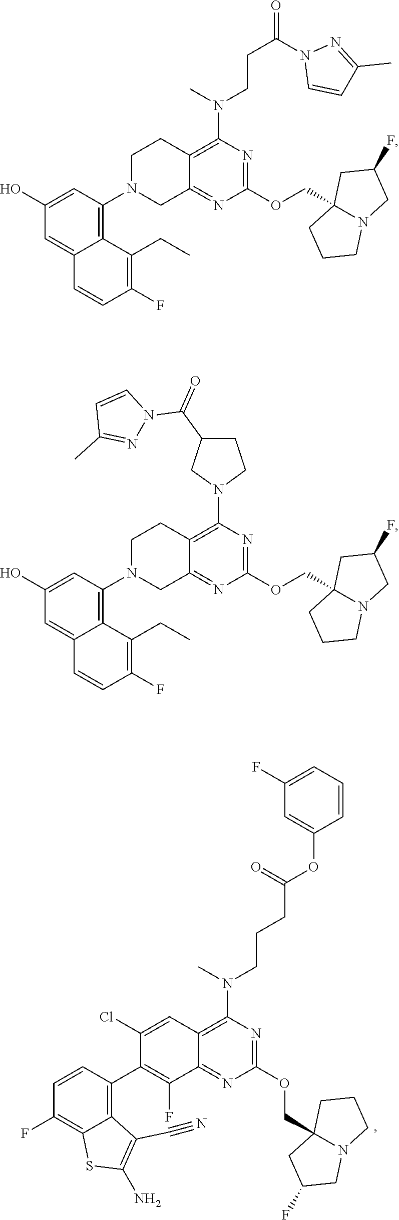 [SMILES: CCc1c(F)ccc2cc(O)cc(N3CCc4c(nc(OC[C@@]56CCCN5C[C@H](F)C6)nc4N(C)CCC(=O)n4ccc(C)n4)C3)c12 ; CCc1c(F)ccc2cc(O)cc(N3CCc4c(nc(OC[C@@]56CCCN5C[C@H](F)C6)nc4N4CCC(C(=O)n5ccc(C)n5)C4)C3)c12 ; CN(CCCC(=O)Oc1cccc(F)c1)c1nc(OC[C@@]23CCCN2C[C@H](F)C3)nc2c(F)c(-c3ccc(F)c4sc(N)c(C#N)c34)c(Cl)cc12] 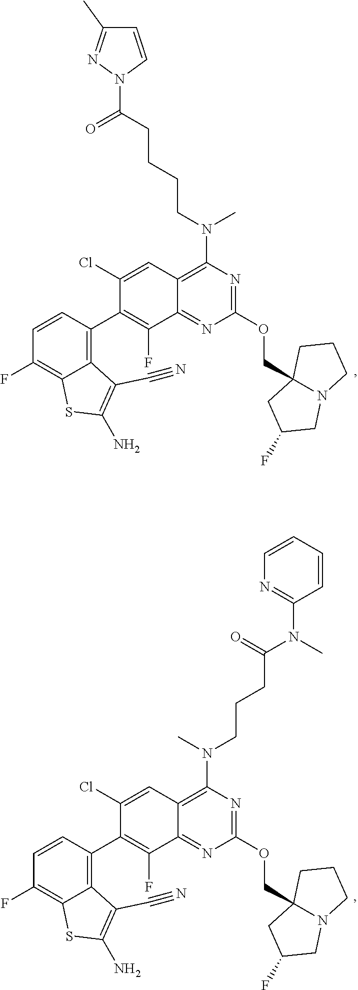 [SMILES: Cc1ccn(C(=O)CCCCN(C)c2nc(OC[C@@]34CCCN3C[C@H](F)C4)nc3c(F)c(-c4ccc(F)c5sc(N)c(C#N)c45)c(Cl)cc23)n1 ; CN(CCCC(=O)N(C)c1ccccn1)c1nc(OC[C@@]23CCCN2C[C@H](F)C3)nc2c(F)c(-c3ccc(F)c4sc(N)c(C#N)c34)c(Cl)cc12] 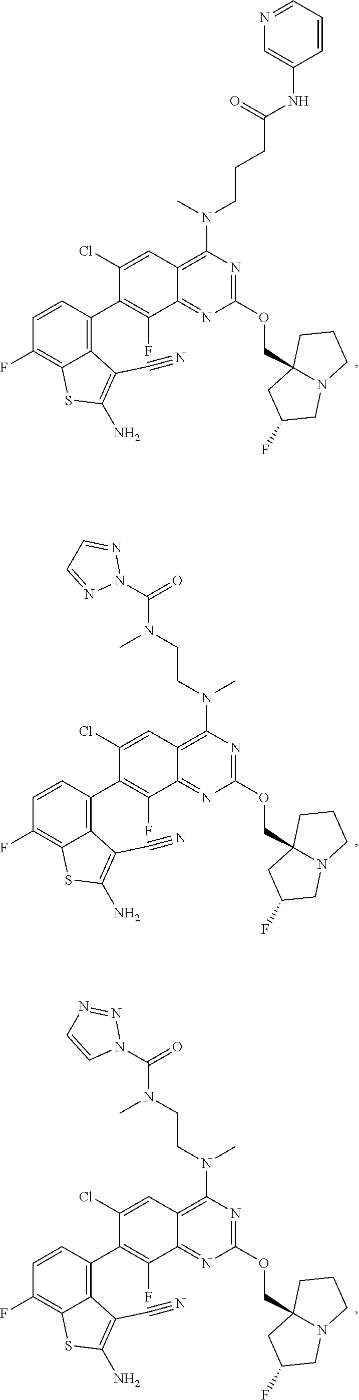 [SMILES: CN(CCCC(=O)Nc1cccnc1)c1nc(OC[C@@]23CCCN2C[C@H](F)C3)nc2c(F)c(-c3ccc(F)c4sc(N)c(C#N)c34)c(Cl)cc12 ; CN(CCN(C)c1nc(OC[C@@]23CCCN2C[C@H](F)C3)nc2c(F)c(-c3ccc(F)c4sc(N)c(C#N)c34)c(Cl)cc12)C(=O)n1nccn1 ; CN(CCN(C)c1nc(OC[C@@]23CCCN2C[C@H](F)C3)nc2c(F)c(-c3ccc(F)c4sc(N)c(C#N)c34)c(Cl)cc12)C(=O)n1ccnn1] 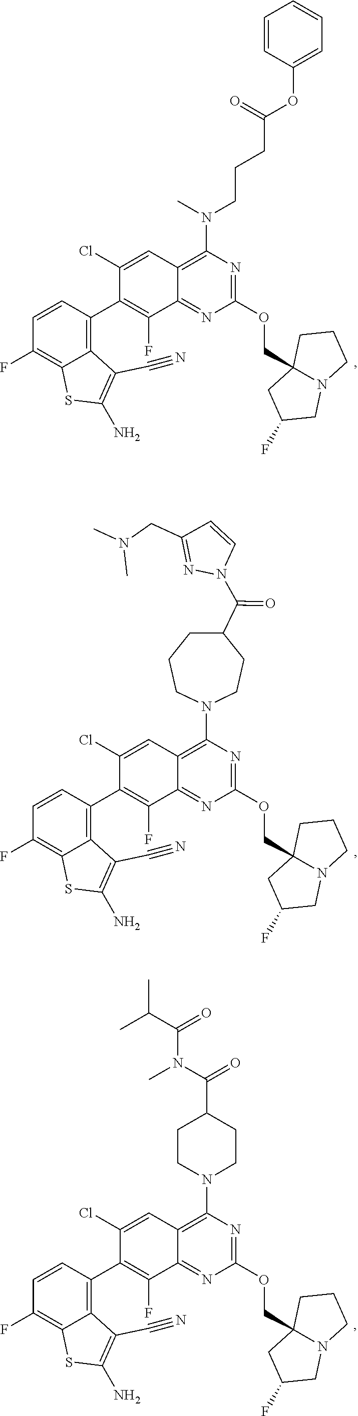 [SMILES: CN(CCCC(=O)Oc1ccccc1)c1nc(OC[C@@]23CCCN2C[C@H](F)C3)nc2c(F)c(-c3ccc(F)c4sc(N)c(C#N)c34)c(Cl)cc12 ; CN(C)Cc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; CC(C)C(=O)N(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1] 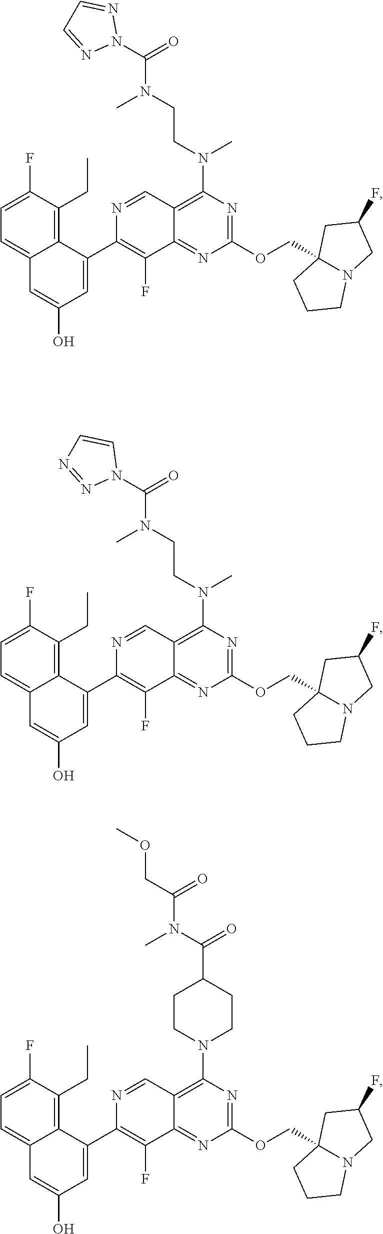 [SMILES: CCc1c(F)ccc2cc(O)cc(-c3ncc4c(N(C)CCN(C)C(=O)n5nccn5)nc(OC[C@@]56CCCN5C[C@H](F)C6)nc4c3F)c12 ; CCc1c(F)ccc2cc(O)cc(-c3ncc4c(N(C)CCN(C)C(=O)n5ccnn5)nc(OC[C@@]56CCCN5C[C@H](F)C6)nc4c3F)c12 ; CCc1c(F)ccc2cc(O)cc(-c3ncc4c(N5CCC(C(=O)N(C)C(=O)COC)CC5)nc(OC[C@@]56CCCN5C[C@H](F)C6)nc4c3F)c12] 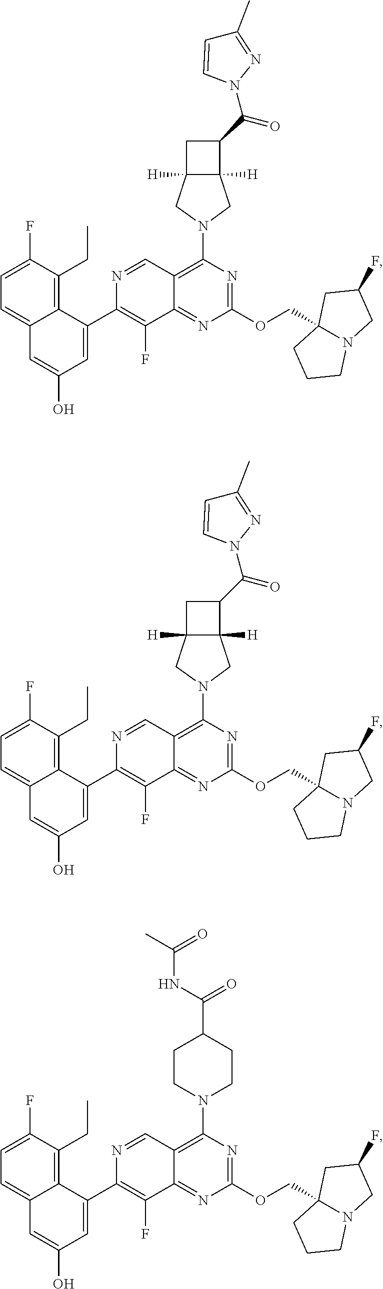 [SMILES: CCc1c(F)ccc2cc(O)cc(-c3ncc4c(N5C[C@H]6C[C@@H](C(=O)n7ccc(C)n7)[C@H]6C5)nc(OC[C@@]56CCCN5C[C@H](F)C6)nc4c3F)c12 ; CCc1c(F)ccc2cc(O)cc(-c3ncc4c(N5C[C@@H]6CC(C(=O)n7ccc(C)n7)[C@@H]6C5)nc(OC[C@@]56CCCN5C[C@H](F)C6)nc4c3F)c12 ; CCc1c(F)ccc2cc(O)cc(-c3ncc4c(N5CCC(C(=O)NC(C)=O)CC5)nc(OC[C@@]56CCCN5C[C@H](F)C6)nc4c3F)c12] 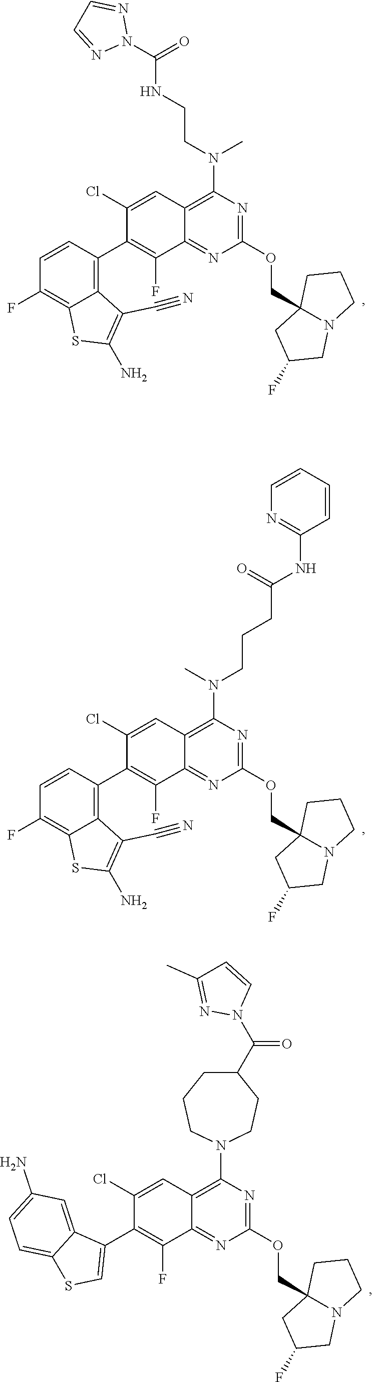 [SMILES: CN(CCNC(=O)n1nccn1)c1nc(OC[C@@]23CCCN2C[C@H](F)C3)nc2c(F)c(-c3ccc(F)c4sc(N)c(C#N)c34)c(Cl)cc12 ; CN(CCCC(=O)Nc1ccccn1)c1nc(OC[C@@]23CCCN2C[C@H](F)C3)nc2c(F)c(-c3ccc(F)c4sc(N)c(C#N)c34)c(Cl)cc12 ; Cc1ccn(C(=O)C2CCCN(c3nc(OC[C@@]45CCCN4C[C@H](F)C5)nc4c(F)c(-c5csc6ccc(N)cc56)c(Cl)cc34)CC2)n1] 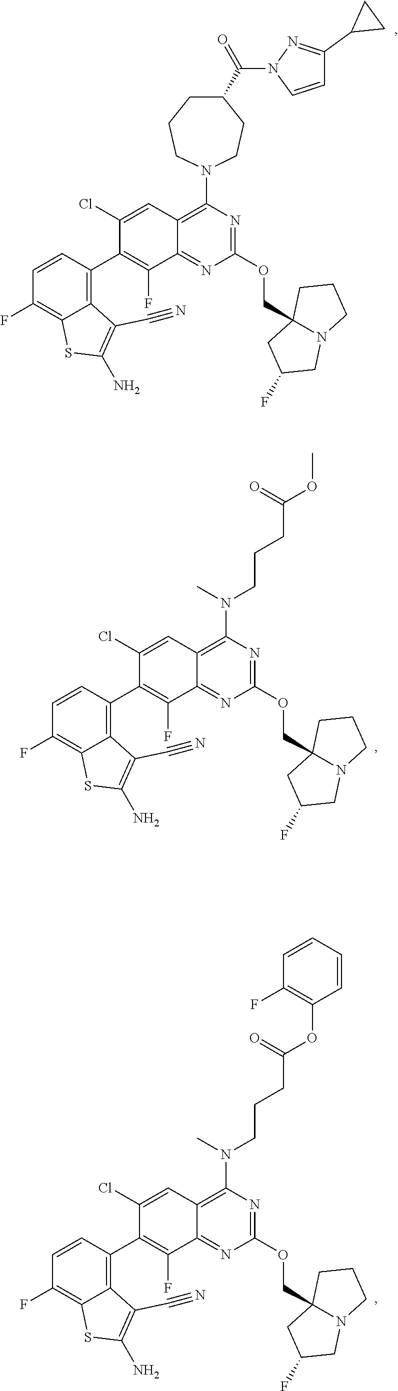 [SMILES: N#Cc1c(N)sc2c(F)ccc(-c3c(Cl)cc4c(N5CCC[C@H](C(=O)n6ccc(C7CC7)n6)CC5)nc(OC[C@@]56CCCN5C[C@H](F)C6)nc4c3F)c12 ; COC(=O)CCCN(C)c1nc(OC[C@@]23CCCN2C[C@H](F)C3)nc2c(F)c(-c3ccc(F)c4sc(N)c(C#N)c34)c(Cl)cc12 ; CN(CCCC(=O)Oc1ccccc1F)c1nc(OC[C@@]23CCCN2C[C@H](F)C3)nc2c(F)c(-c3ccc(F)c4sc(N)c(C#N)c34)c(Cl)cc12] 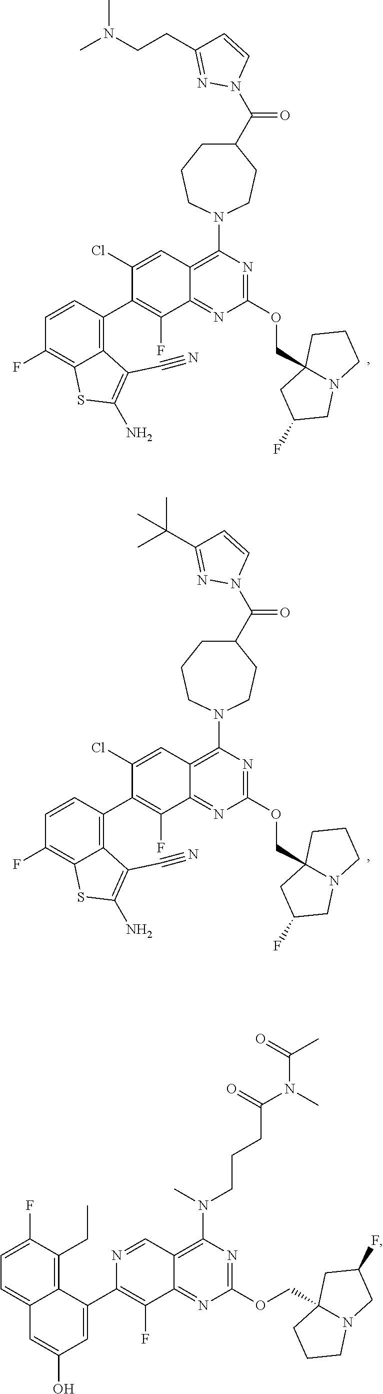 [SMILES: CN(C)CCc1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; CC(C)(C)c1ccn(C(=O)C2CCCN(c3nc(OC[C@@]45CCCN4C[C@H](F)C5)nc4c(F)c(-c5ccc(F)c6sc(N)c(C#N)c56)c(Cl)cc34)CC2)n1 ; CCc1c(F)ccc2cc(O)cc(-c3ncc4c(N(C)CCCC(=O)N(C)C(C)=O)nc(OC[C@@]56CCCN5C[C@H](F)C6)nc4c3F)c12] 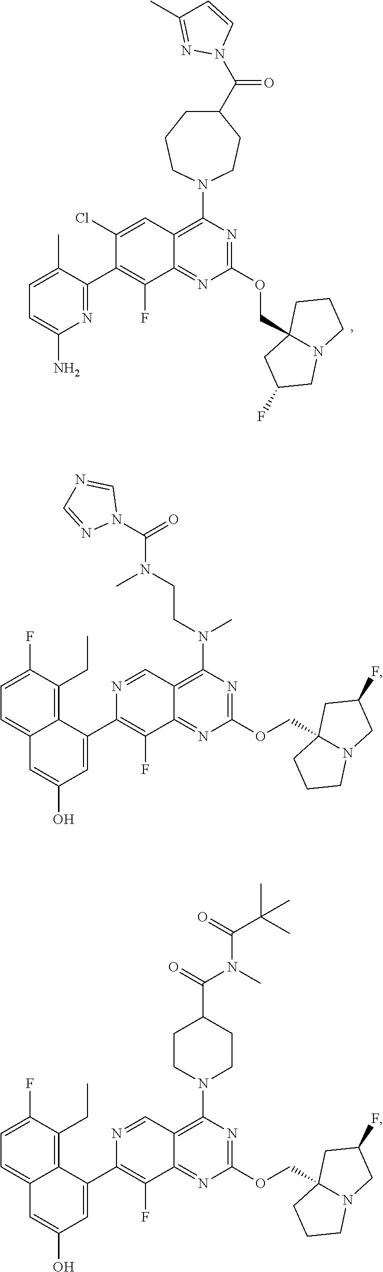 [SMILES: Cc1ccn(C(=O)C2CCCN(c3nc(OC[C@@]45CCCN4C[C@H](F)C5)nc4c(F)c(-c5nc(N)ccc5C)c(Cl)cc34)CC2)n1 ; CCc1c(F)ccc2cc(O)cc(-c3ncc4c(N(C)CCN(C)C(=O)n5cncn5)nc(OC[C@@]56CCCN5C[C@H](F)C6)nc4c3F)c12 ; CCc1c(F)ccc2cc(O)cc(-c3ncc4c(N5CCC(C(=O)N(C)C(=O)C(C)(C)C)CC5)nc(OC[C@@]56CCCN5C[C@H](F)C6)nc4c3F)c12] 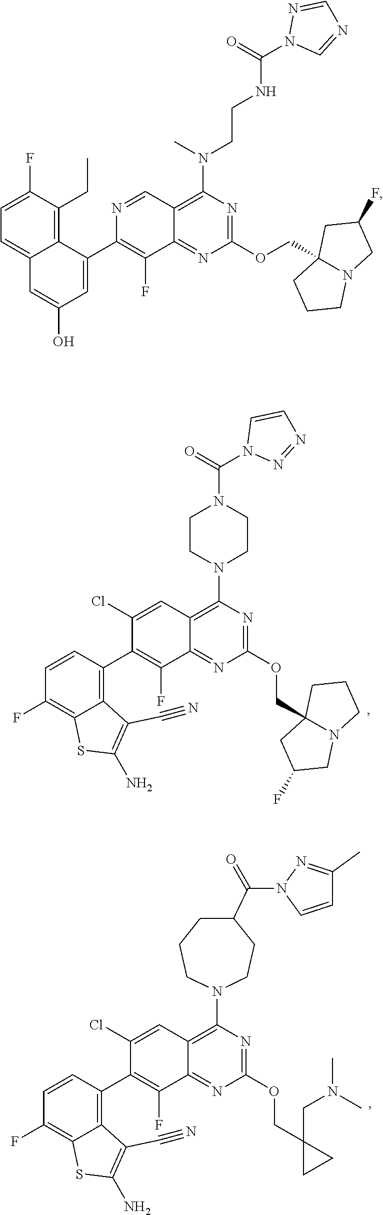 [SMILES: CCc1c(F)ccc2cc(O)cc(-c3ncc4c(N(C)CCNC(=O)n5cncn5)nc(OC[C@@]56CCCN5C[C@H](F)C6)nc4c3F)c12 ; N#Cc1c(N)sc2c(F)ccc(-c3c(Cl)cc4c(N5CCN(C(=O)n6ccnn6)CC5)nc(OC[C@@]56CCCN5C[C@H](F)C6)nc4c3F)c12 ; Cc1ccn(C(=O)C2CCCN(c3nc(OCC4(CN(C)C)CC4)nc4c(F)c(-c5ccc(F)c6sc(N)c(C#N)c56)c(Cl)cc34)CC2)n1] 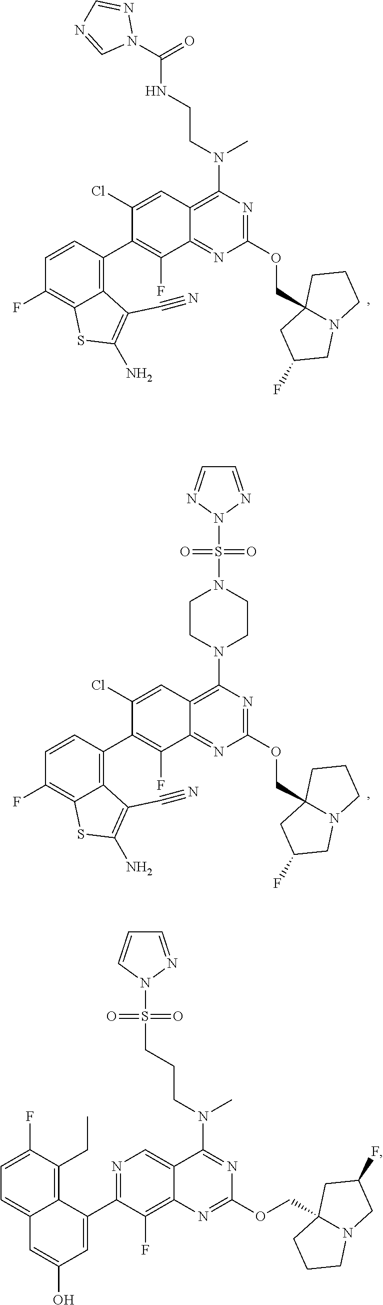 [SMILES: CN(CCNC(=O)n1cncn1)c1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3c(Cl)cc4c(N5CCN(S(=O)(=O)n6nccn6)CC5)nc(OC[C@@]56CCCN5C[C@H](F)C6)nc4c3F)c12 ; CCc1c(F)ccc2cc(O)cc(-c3ncc4c(N(C)CCCS(=O)(=O)n5cccn5)nc(OC[C@@]56CCCN5C[C@H](F)C6)nc4c3F)c12] 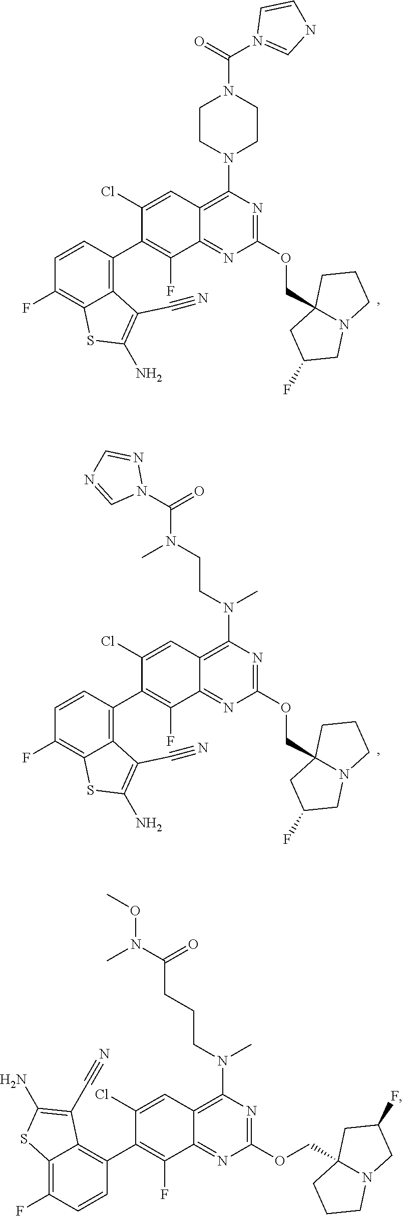 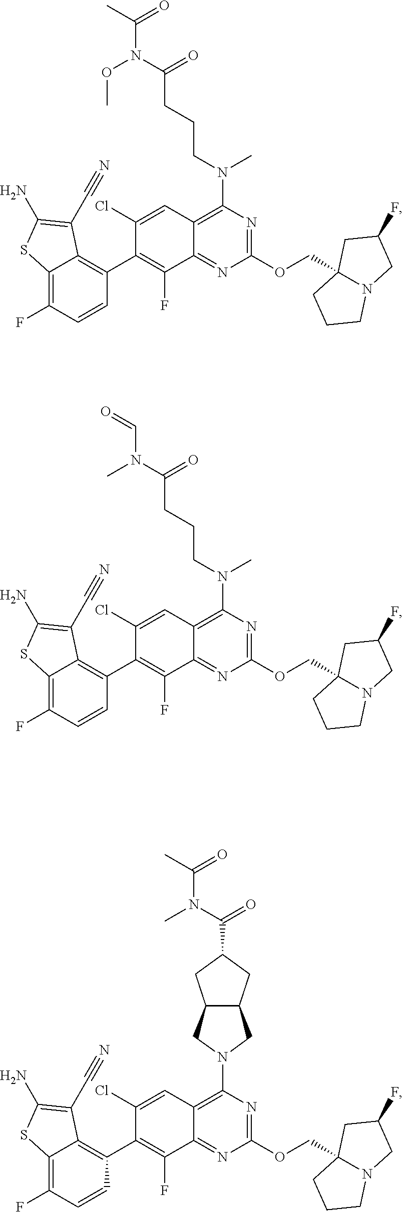 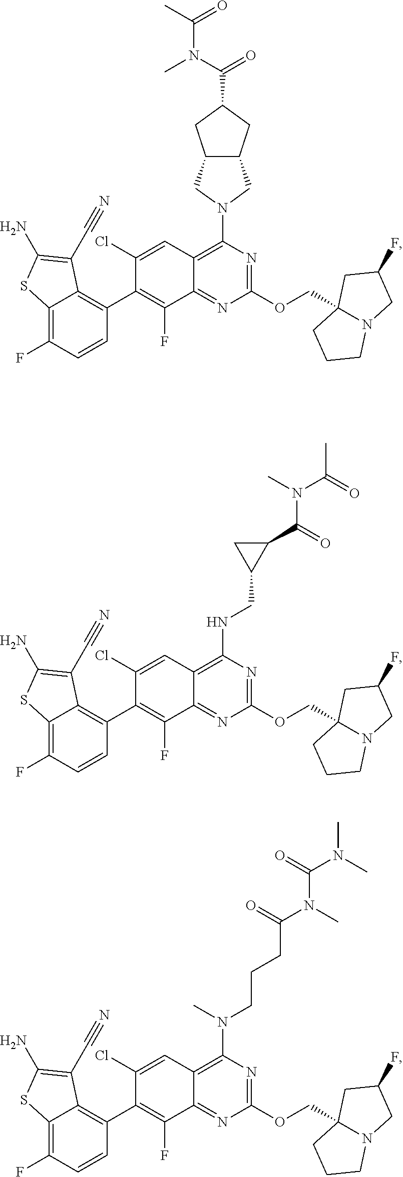 [SMILES: CC(=O)N(C)C(=O)[C@@H]1C[C@@H]2CN(c3nc(OC[C@@]45CCCN4C[C@H](F)C5)nc4c(F)c(-c5ccc(F)c6sc(N)c(C#N)c56)c(Cl)cc34)C[C@@H]2C1 ; CC(=O)N(C)C(=O)[C@@H]1C[C@H]1CNc1nc(OC[C@@]23CCCN2C[C@H](F)C3)nc2c(F)c(-c3ccc(F)c4sc(N)c(C#N)c34)c(Cl)cc12 ; CN(C)C(=O)N(C)C(=O)CCCN(C)c1nc(OC[C@@]23CCCN2C[C@H](F)C3)nc2c(F)c(-c3ccc(F)c4sc(N)c(C#N)c34)c(Cl)cc12] 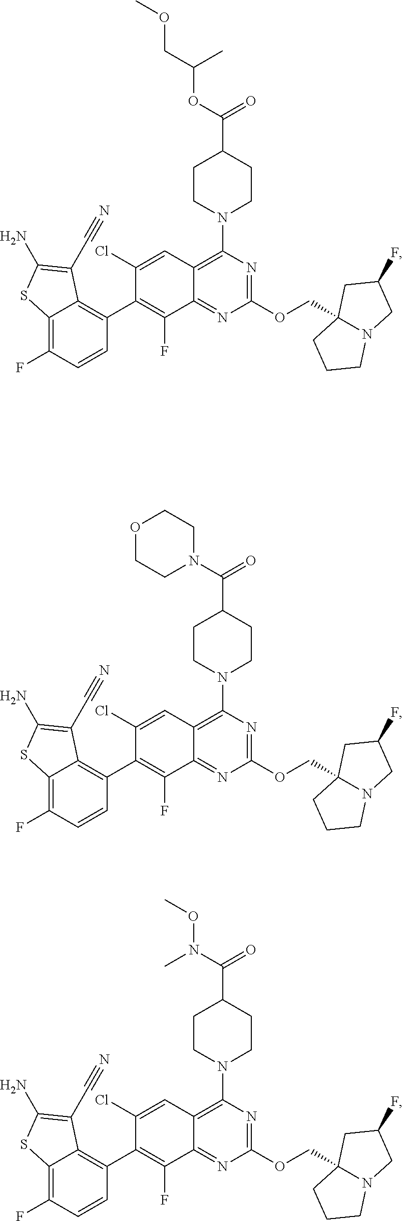 [SMILES: COCC(C)OC(=O)C1CCN(c2nc(OC[C@@]34CCCN3C[C@H](F)C4)nc3c(F)c(-c4ccc(F)c5sc(N)c(C#N)c45)c(Cl)cc23)CC1 ; N#Cc1c(N)sc2c(F)ccc(-c3c(Cl)cc4c(N5CCC(C(=O)N6CCOCC6)CC5)nc(OC[C@@]56CCCN5C[C@H](F)C6)nc4c3F)c12 ; CON(C)C(=O)C1CCN(c2nc(OC[C@@]34CCCN3C[C@H](F)C4)nc3c(F)c(-c4ccc(F)c5sc(N)c(C#N)c45)c(Cl)cc23)CC1] 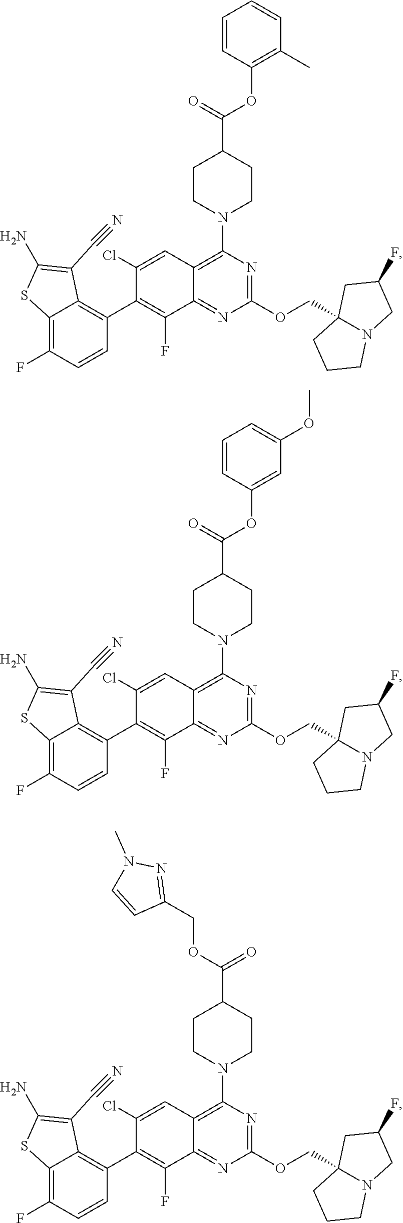 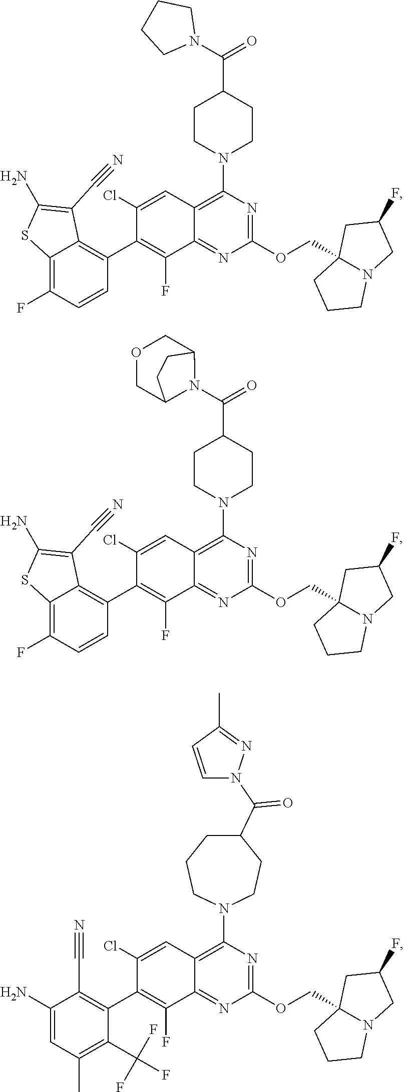 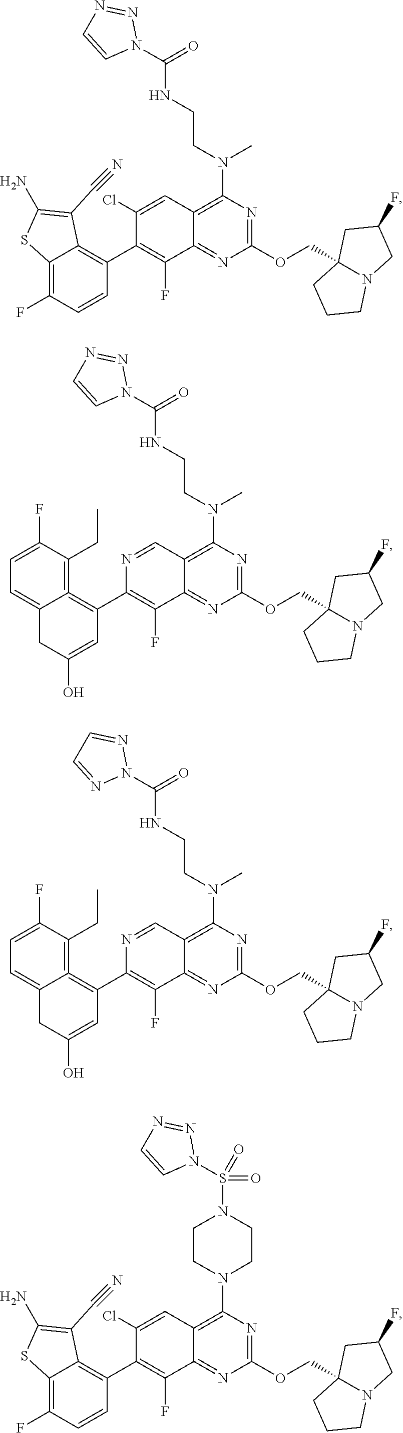 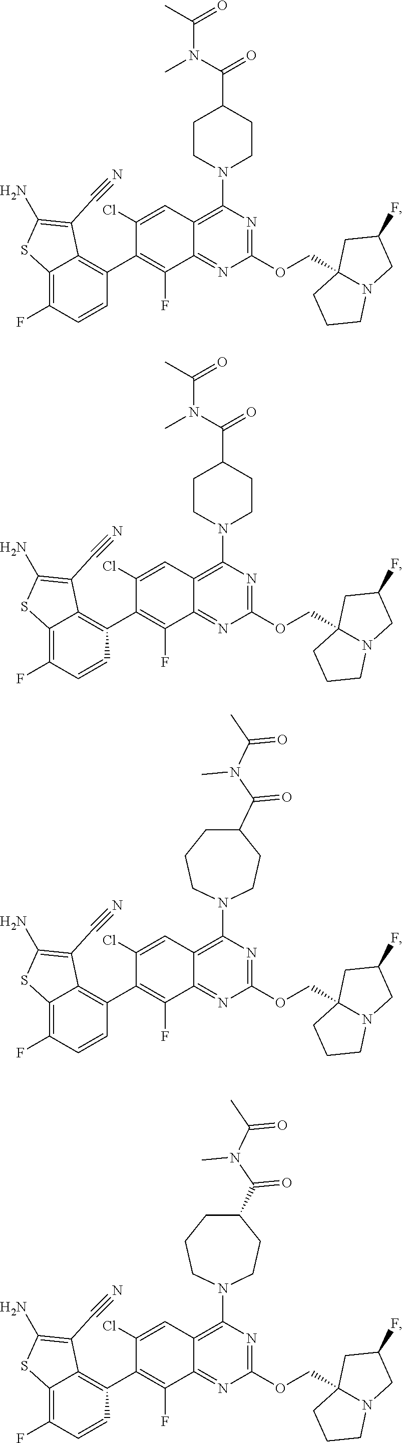 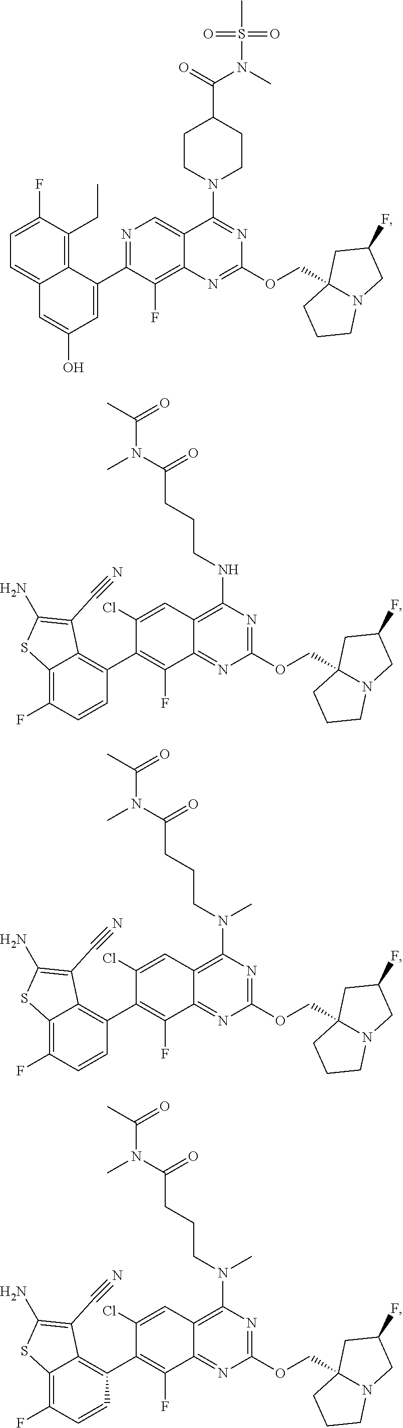 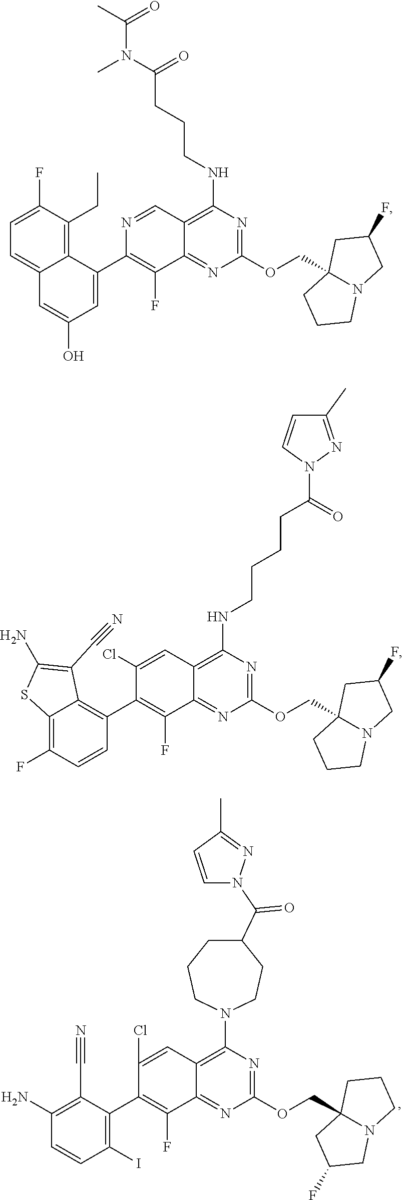 [SMILES: CCc1c(F)ccc2cc(O)cc(-c3ncc4c(NCCCC(=O)N(C)C(C)=O)nc(OC[C@@]56CCCN5C[C@H](F)C6)nc4c3F)c12 ; Cc1ccn(C(=O)CCCCNc2nc(OC[C@@]34CCCN3C[C@H](F)C4)nc3c(F)c(-c4ccc(F)c5sc(N)c(C#N)c45)c(Cl)cc23)n1 ; Cc1ccn(C(=O)C2CCCN(c3nc(OC[C@@]45CCCN4C[C@H](F)C5)nc4c(F)c(-c5c(I)ccc(N)c5C#N)c(Cl)cc34)CC2)n1] 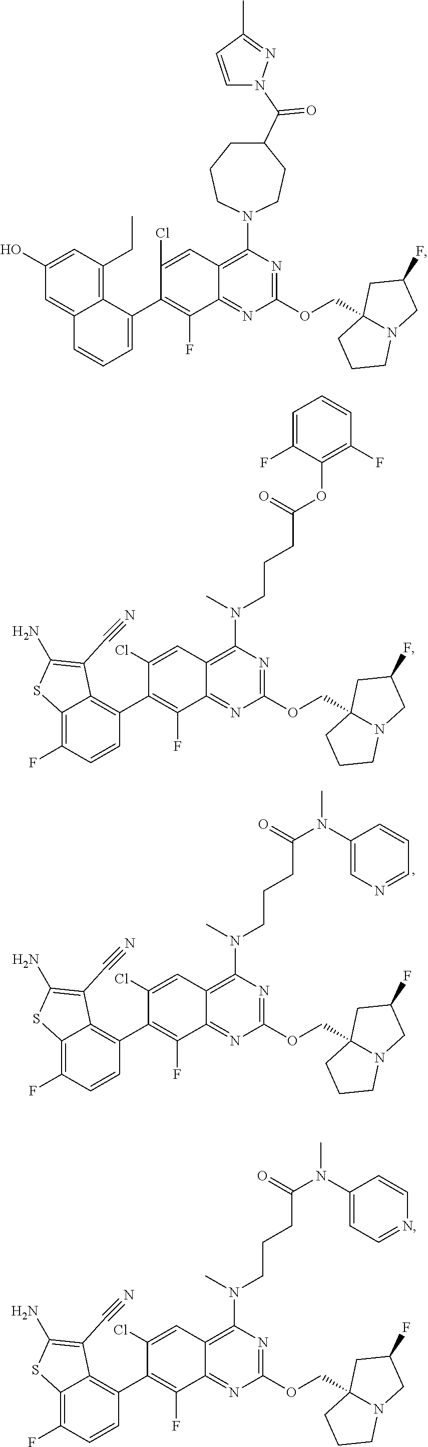 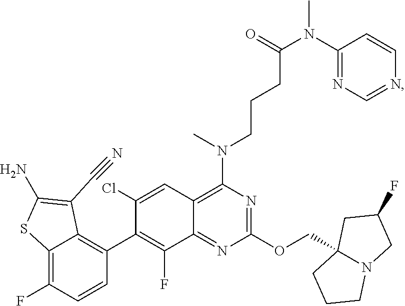 [SMILES: CN(CCCC(=O)N(C)c1ccncn1)c1nc(OC[C@@]23CCCN2C[C@H](F)C3)nc2c(F)c(-c3ccc(F)c4sc(N)c(C#N)c34)c(Cl)cc12] 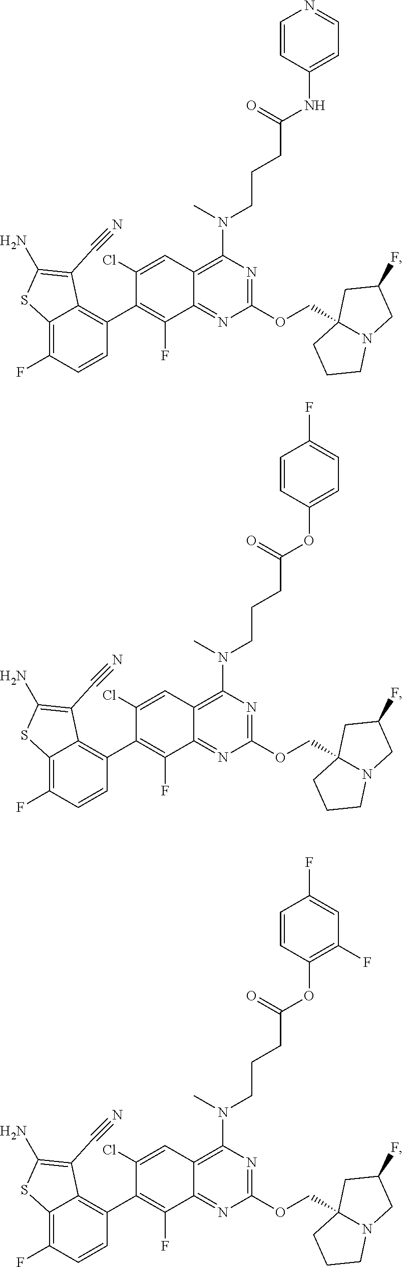 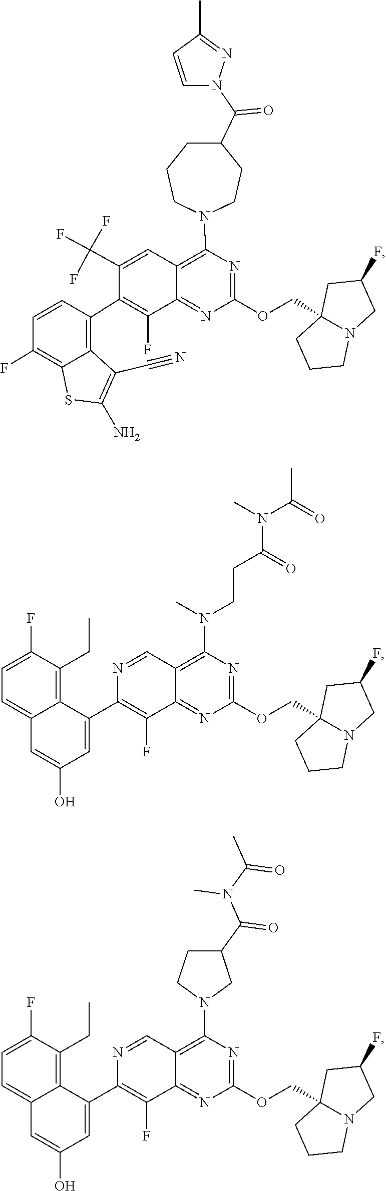 [SMILES: Cc1ccn(C(=O)C2CCCN(c3nc(OC[C@@]45CCCN4C[C@H](F)C5)nc4c(F)c(-c5ccc(F)c6sc(N)c(C#N)c56)c(C(F)(F)F)cc34)CC2)n1 ; CCc1c(F)ccc2cc(O)cc(-c3ncc4c(N(C)CCC(=O)N(C)C(C)=O)nc(OC[C@@]56CCCN5C[C@H](F)C6)nc4c3F)c12 ; CCc1c(F)ccc2cc(O)cc(-c3ncc4c(N5CCC(C(=O)N(C)C(C)=O)C5)nc(OC[C@@]56CCCN5C[C@H](F)C6)nc4c3F)c12] 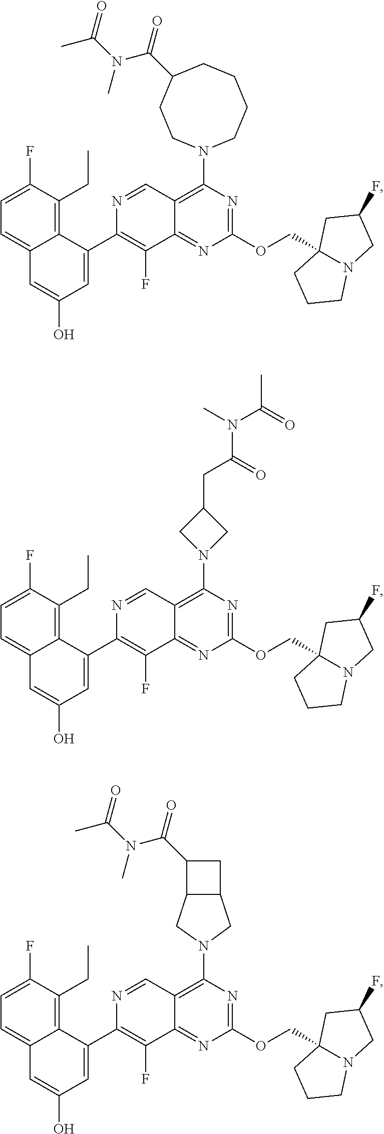 [SMILES: CCc1c(F)ccc2cc(O)cc(-c3ncc4c(N5CCCCC(C(=O)N(C)C(C)=O)CC5)nc(OC[C@@]56CCCN5C[C@H](F)C6)nc4c3F)c12 ; CCc1c(F)ccc2cc(O)cc(-c3ncc4c(N5CC(CC(=O)N(C)C(C)=O)C5)nc(OC[C@@]56CCCN5C[C@H](F)C6)nc4c3F)c12 ; CCc1c(F)ccc2cc(O)cc(-c3ncc4c(N5CC6CC(C(=O)N(C)C(C)=O)C6C5)nc(OC[C@@]56CCCN5C[C@H](F)C6)nc4c3F)c12] 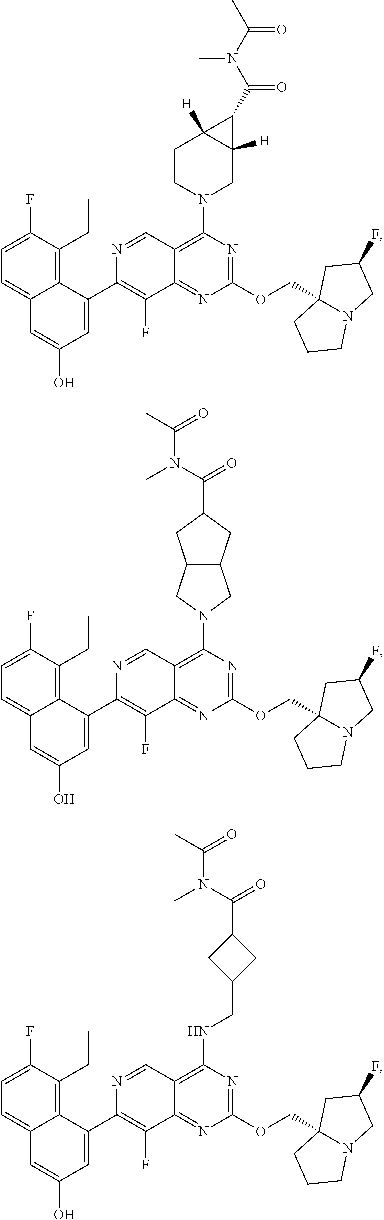 [SMILES: CCc1c(F)ccc2cc(O)cc(-c3ncc4c(N5CC[C@H]6[C@@H](C5)[C@H]6C(=O)N(C)C(C)=O)nc(OC[C@@]56CCCN5C[C@H](F)C6)nc4c3F)c12 ; CCc1c(F)ccc2cc(O)cc(-c3ncc4c(N5CC6CC(C(=O)N(C)C(C)=O)CC6C5)nc(OC[C@@]56CCCN5C[C@H](F)C6)nc4c3F)c12 ; CCc1c(F)ccc2cc(O)cc(-c3ncc4c(NCC5CC(C(=O)N(C)C(C)=O)C5)nc(OC[C@@]56CCCN5C[C@H](F)C6)nc4c3F)c12] 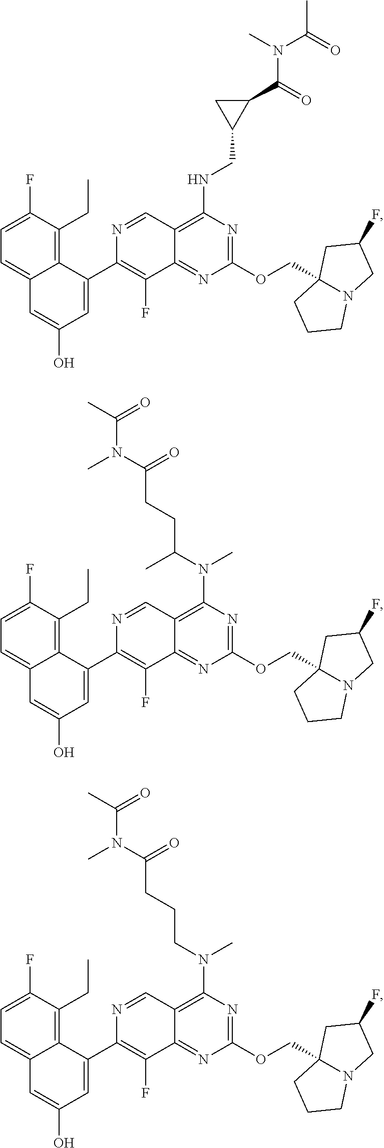 [SMILES: CCc1c(F)ccc2cc(O)cc(-c3ncc4c(NC[C@@H]5C[C@H]5C(=O)N(C)C(C)=O)nc(OC[C@@]56CCCN5C[C@H](F)C6)nc4c3F)c12 ; CCc1c(F)ccc2cc(O)cc(-c3ncc4c(N(C)C(C)CCC(=O)N(C)C(C)=O)nc(OC[C@@]56CCCN5C[C@H](F)C6)nc4c3F)c12 ; CCc1c(F)ccc2cc(O)cc(-c3ncc4c(N(C)CCCC(=O)N(C)C(C)=O)nc(OC[C@@]56CCCN5C[C@H](F)C6)nc4c3F)c12] 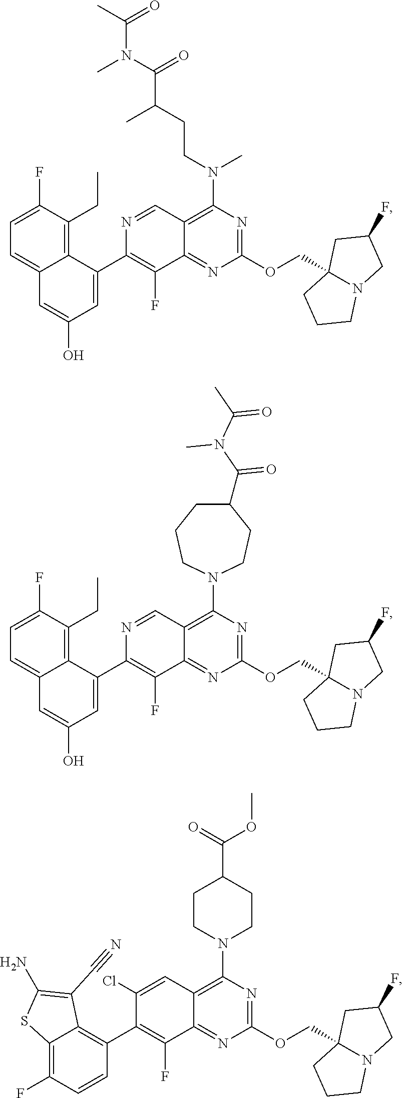 [SMILES: CCc1c(F)ccc2cc(O)cc(-c3ncc4c(N(C)CCC(C)C(=O)N(C)C(C)=O)nc(OC[C@@]56CCCN5C[C@H](F)C6)nc4c3F)c12 ; CCc1c(F)ccc2cc(O)cc(-c3ncc4c(N5CCCC(C(=O)N(C)C(C)=O)CC5)nc(OC[C@@]56CCCN5C[C@H](F)C6)nc4c3F)c12 ; COC(=O)C1CCN(c2nc(OC[C@@]34CCCN3C[C@H](F)C4)nc3c(F)c(-c4ccc(F)c5sc(N)c(C#N)c45)c(Cl)cc23)CC1] 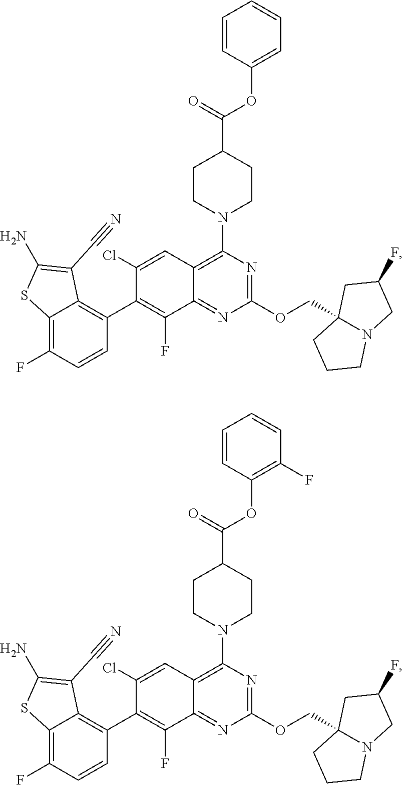 [SMILES: N#Cc1c(N)sc2c(F)ccc(-c3c(Cl)cc4c(N5CCC(C(=O)Oc6ccccc6)CC5)nc(OC[C@@]56CCCN5C[C@H](F)C6)nc4c3F)c12 ; N#Cc1c(N)sc2c(F)ccc(-c3c(Cl)cc4c(N5CCC(C(=O)Oc6ccccc6F)CC5)nc(OC[C@@]56CCCN5C[C@H](F)C6)nc4c3F)c12] 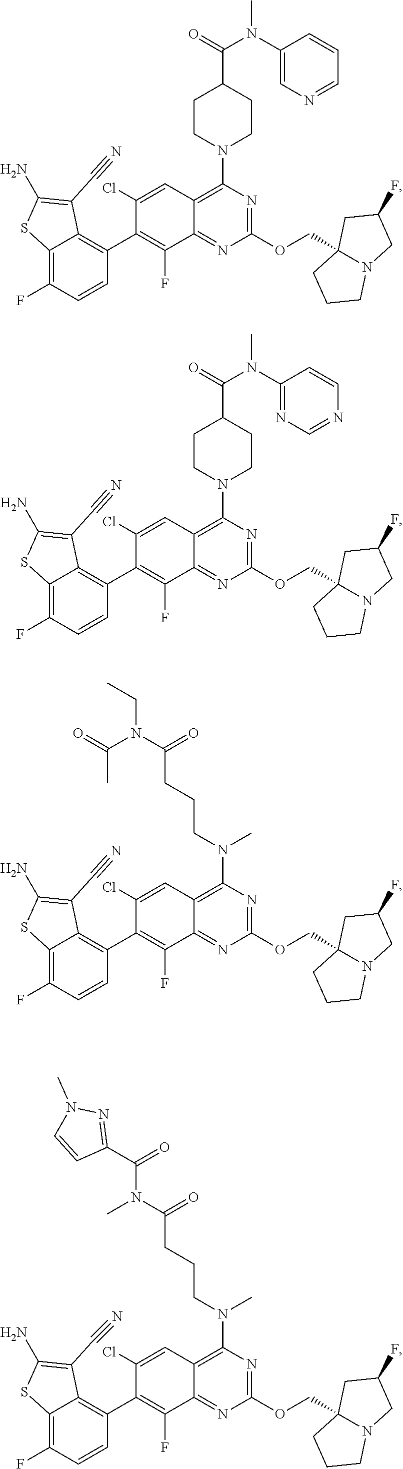 [SMILES: CN(C(=O)C1CCN(c2nc(OC[C@@]34CCCN3C[C@H](F)C4)nc3c(F)c(-c4ccc(F)c5sc(N)c(C#N)c45)c(Cl)cc23)CC1)c1cccnc1 ; CN(C(=O)C1CCN(c2nc(OC[C@@]34CCCN3C[C@H](F)C4)nc3c(F)c(-c4ccc(F)c5sc(N)c(C#N)c45)c(Cl)cc23)CC1)c1ccncn1 ; CCN(C(C)=O)C(=O)CCCN(C)c1nc(OC[C@@]23CCCN2C[C@H](F)C3)nc2c(F)c(-c3ccc(F)c4sc(N)c(C#N)c34)c(Cl)cc12 ; CN(C(=O)CCCN(C)c1nc(OC[C@@]23CCCN2C[C@H](F)C3)nc2c(F)c(-c3ccc(F)c4sc(N)c(C#N)c34)c(Cl)cc12)C(=O)c1ccn(C)n1] 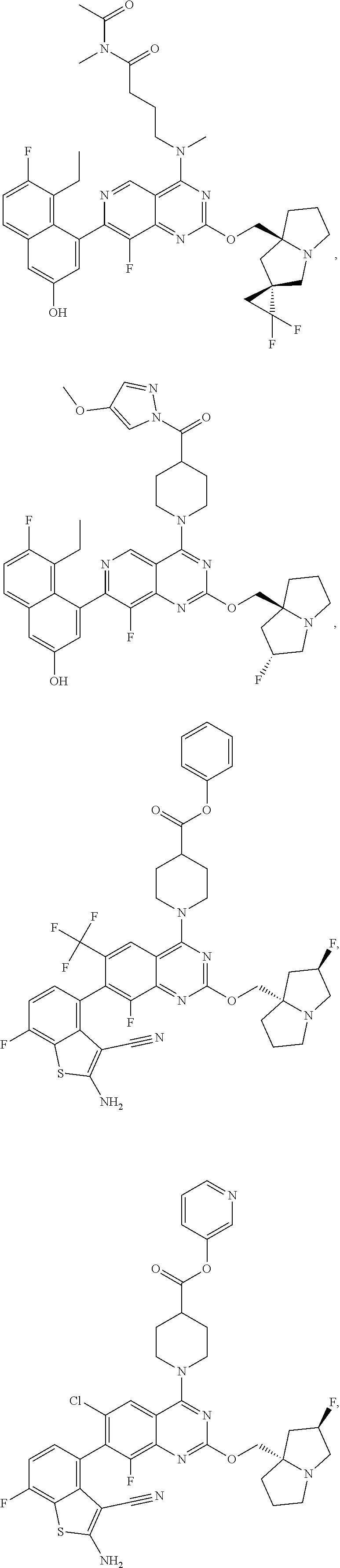 [SMILES: CCc1c(F)ccc2cc(O)cc(-c3ncc4c(N(C)CCCC(=O)N(C)C(C)=O)nc(OC[C@@]56CCCN5C[C@@]5(CC5(F)F)C6)nc4c3F)c12 ; CCc1c(F)ccc2cc(O)cc(-c3ncc4c(N5CCC(C(=O)n6cc(OC)cn6)CC5)nc(OC[C@@]56CCCN5C[C@H](F)C6)nc4c3F)c12 ; N#Cc1c(N)sc2c(F)ccc(-c3c(C(F)(F)F)cc4c(N5CCC(C(=O)Oc6ccccc6)CC5)nc(OC[C@@]56CCCN5C[C@H](F)C6)nc4c3F)c12 ; N#Cc1c(N)sc2c(F)ccc(-c3c(Cl)cc4c(N5CCC(C(=O)Oc6cccnc6)CC5)nc(OC[C@@]56CCCN5C[C@H](F)C6)nc4c3F)c12] 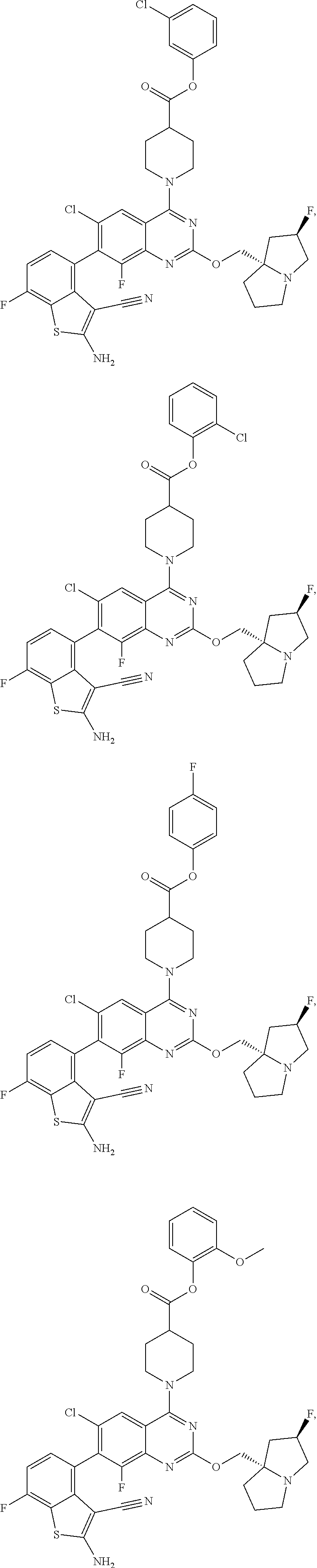 [SMILES: N#Cc1c(N)sc2c(F)ccc(-c3c(Cl)cc4c(N5CCC(C(=O)Oc6cccc(Cl)c6)CC5)nc(OC[C@@]56CCCN5C[C@H](F)C6)nc4c3F)c12 ; N#Cc1c(N)sc2c(F)ccc(-c3c(Cl)cc4c(N5CCC(C(=O)Oc6ccccc6Cl)CC5)nc(OC[C@@]56CCCN5C[C@H](F)C6)nc4c3F)c12 ; N#Cc1c(N)sc2c(F)ccc(-c3c(Cl)cc4c(N5CCC(C(=O)Oc6ccc(F)cc6)CC5)nc(OC[C@@]56CCCN5C[C@H](F)C6)nc4c3F)c12 ; COc1ccccc1OC(=O)C1CCN(c2nc(OC[C@@]34CCCN3C[C@H](F)C4)nc3c(F)c(-c4ccc(F)c5sc(N)c(C#N)c45)c(Cl)cc23)CC1] 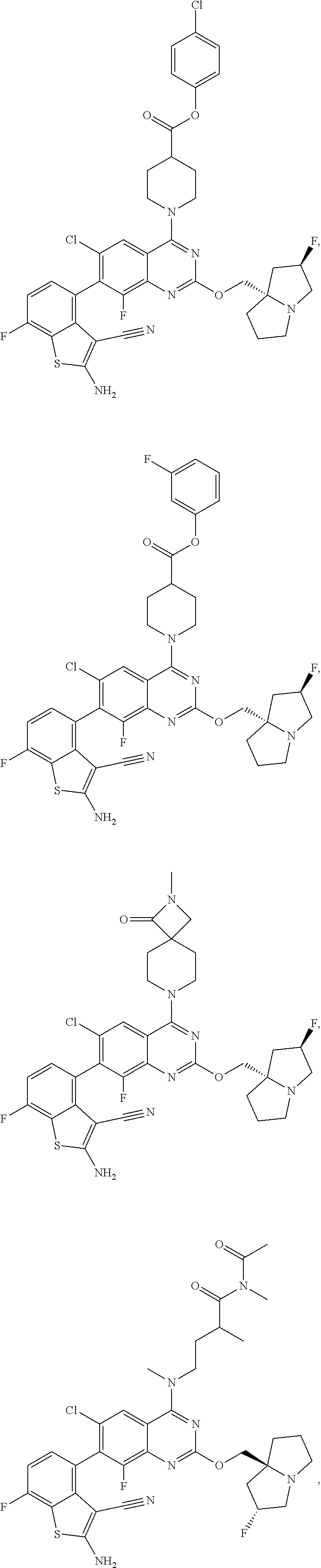 [SMILES: N#Cc1c(N)sc2c(F)ccc(-c3c(Cl)cc4c(N5CCC(C(=O)Oc6ccc(Cl)cc6)CC5)nc(OC[C@@]56CCCN5C[C@H](F)C6)nc4c3F)c12 ; N#Cc1c(N)sc2c(F)ccc(-c3c(Cl)cc4c(N5CCC(C(=O)Oc6cccc(F)c6)CC5)nc(OC[C@@]56CCCN5C[C@H](F)C6)nc4c3F)c12 ; CN1CC2(CCN(c3nc(OC[C@@]45CCCN4C[C@H](F)C5)nc4c(F)c(-c5ccc(F)c6sc(N)c(C#N)c56)c(Cl)cc34)CC2)C1=O ; CC(=O)N(C)C(=O)C(C)CCN(C)c1nc(OC[C@@]23CCCN2C[C@H](F)C3)nc2c(F)c(-c3ccc(F)c4sc(N)c(C#N)c34)c(Cl)cc12] 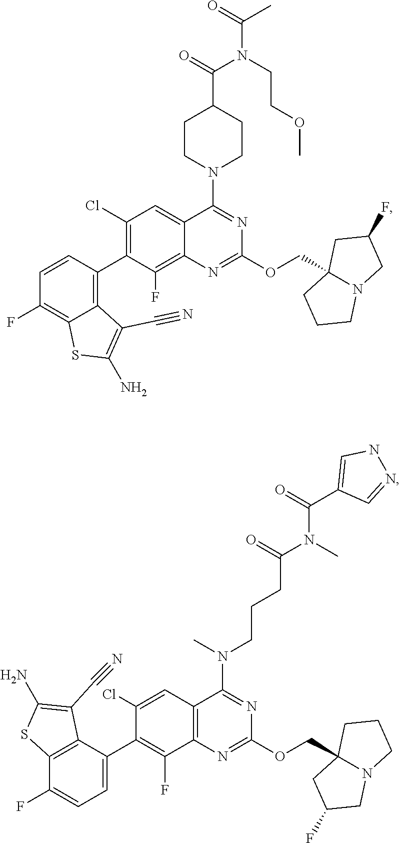 [SMILES: COCCN(C(C)=O)C(=O)C1CCN(c2nc(OC[C@@]34CCCN3C[C@H](F)C4)nc3c(F)c(-c4ccc(F)c5sc(N)c(C#N)c45)c(Cl)cc23)CC1 ; CN(C(=O)CCCN(C)c1nc(OC[C@@]23CCCN2C[C@H](F)C3)nc2c(F)c(-c3ccc(F)c4sc(N)c(C#N)c34)c(Cl)cc12)C(=O)c1cn[nH]c1] 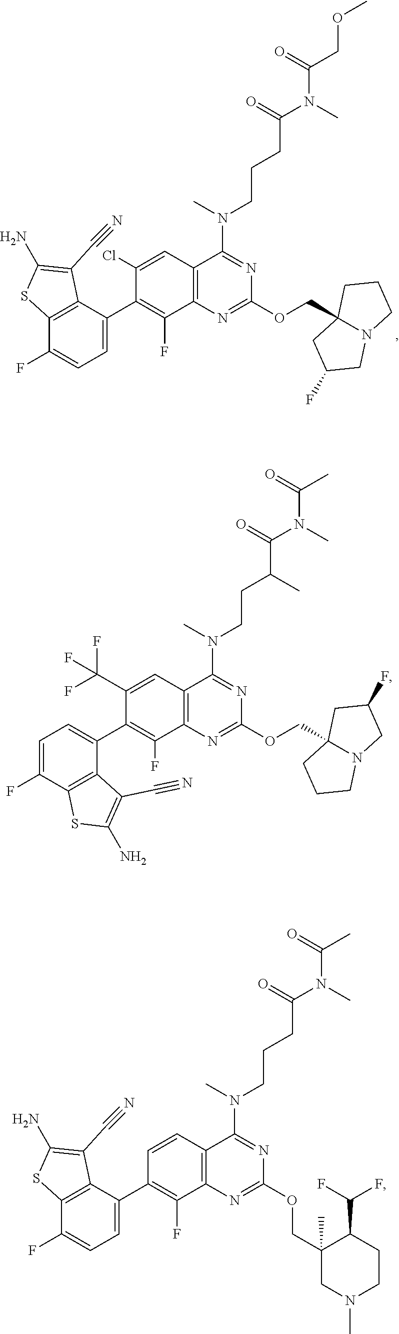 [SMILES: COCC(=O)N(C)C(=O)CCCN(C)c1nc(OC[C@@]23CCCN2C[C@H](F)C3)nc2c(F)c(-c3ccc(F)c4sc(N)c(C#N)c34)c(Cl)cc12 ; CC(=O)N(C)C(=O)C(C)CCN(C)c1nc(OC[C@@]23CCCN2C[C@H](F)C3)nc2c(F)c(-c3ccc(F)c4sc(N)c(C#N)c34)c(C(F)(F)F)cc12 ; CC(=O)N(C)C(=O)CCCN(C)c1nc(OC[C@]2(C)CN(C)CC[C@@H]2C(F)F)nc2c(F)c(-c3ccc(F)c4sc(N)c(C#N)c34)ccc12] 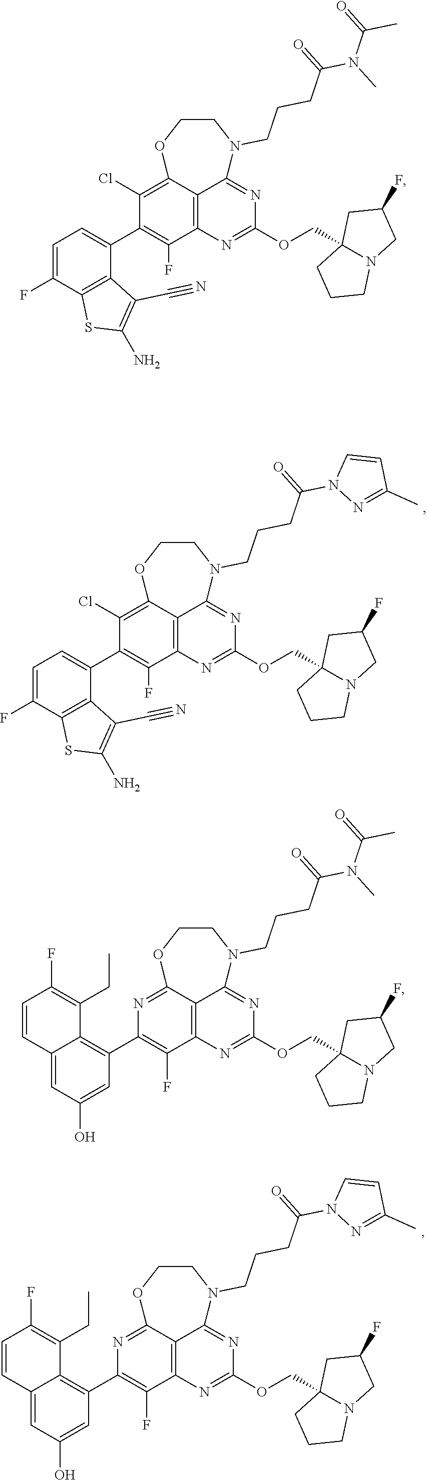 [SMILES: CC(=O)N(C)C(=O)CCCN1CCOc2c(Cl)c(-c3ccc(F)c4sc(N)c(C#N)c34)c(F)c3nc(OC[C@@]45CCCN4C[C@H](F)C5)nc1c23 ; Cc1ccn(C(=O)CCCN2CCOc3c(Cl)c(-c4ccc(F)c5sc(N)c(C#N)c45)c(F)c4nc(OC[C@@]56CCCN5C[C@H](F)C6)nc2c34)n1 ; CCc1c(F)ccc2cc(O)cc(-c3nc4c5c(nc(OC[C@@]67CCCN6C[C@H](F)C7)nc5c3F)N(CCCC(=O)N(C)C(C)=O)CCO4)c12 ; CCc1c(F)ccc2cc(O)cc(-c3nc4c5c(nc(OC[C@@]67CCCN6C[C@H](F)C7)nc5c3F)N(CCCC(=O)n3ccc(C)n3)CCO4)c12] 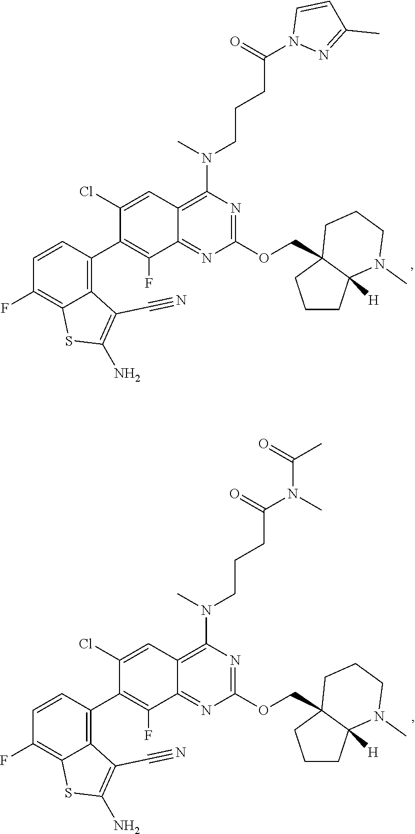 [SMILES: Cc1ccn(C(=O)CCCN(C)c2nc(OC[C@]34CCC[C@H]3N(C)CCC4)nc3c(F)c(-c4ccc(F)c5sc(N)c(C#N)c45)c(Cl)cc23)n1 ; CC(=O)N(C)C(=O)CCCN(C)c1nc(OC[C@]23CCC[C@H]2N(C)CCC3)nc2c(F)c(-c3ccc(F)c4sc(N)c(C#N)c34)c(Cl)cc12] 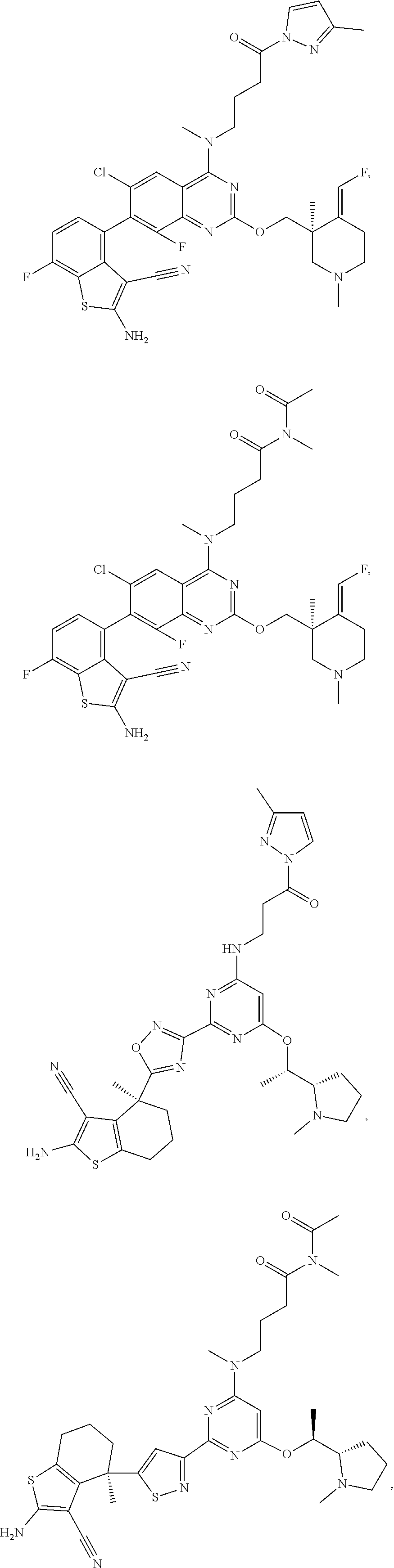 [SMILES: Cc1ccn(C(=O)CCCN(C)c2nc(OC[C@]3(C)CN(C)CC/C3=C\F)nc3c(F)c(-c4ccc(F)c5sc(N)c(C#N)c45)c(Cl)cc23)n1 ; CC(=O)N(C)C(=O)CCCN(C)c1nc(OC[C@]2(C)CN(C)CC/C2=C\F)nc2c(F)c(-c3ccc(F)c4sc(N)c(C#N)c34)c(Cl)cc12 ; Cc1ccn(C(=O)CCNc2cc(O[C@@H](C)[C@@H]3CCCN3C)nc(-c3noc([C@@]4(C)CCCc5sc(N)c(C#N)c54)n3)n2)n1 ; CC(=O)N(C)C(=O)CCCN(C)c1cc(O[C@@H](C)[C@@H]2CCCN2C)nc(-c2cc([C@]3(C)CCCc4sc(N)c(C#N)c43)sn2)n1] 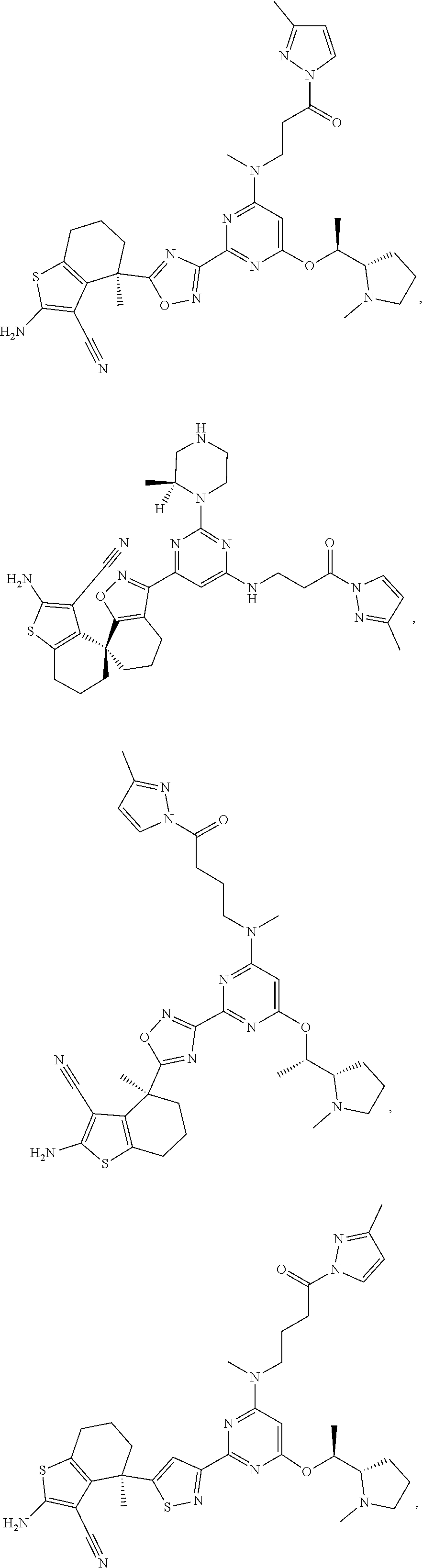 [SMILES: Cc1ccn(C(=O)CCN(C)c2cc(O[C@@H](C)[C@@H]3CCCN3C)nc(-c3noc([C@]4(C)CCCc5sc(N)c(C#N)c54)n3)n2)n1 ; Cc1ccn(C(=O)CCNc2cc(-c3noc4c3CCC[C@@]43CCCc4sc(N)c(C#N)c43)nc(N3CCNC[C@@H]3C)n2)n1 ; Cc1ccn(C(=O)CCCN(C)c2cc(O[C@@H](C)[C@@H]3CCCN3C)nc(-c3noc([C@@]4(C)CCCc5sc(N)c(C#N)c54)n3)n2)n1 ; Cc1ccn(C(=O)CCCN(C)c2cc(O[C@@H](C)[C@@H]3CCCN3C)nc(-c3cc([C@]4(C)CCCc5sc(N)c(C#N)c54)sn3)n2)n1] 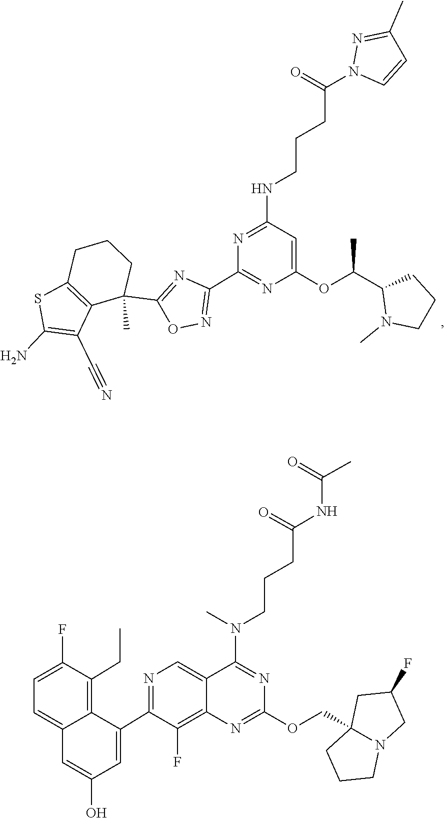 [SMILES: Cc1ccn(C(=O)CCCNc2cc(O[C@@H](C)[C@@H]3CCCN3C)nc(-c3noc([C@]4(C)CCCc5sc(N)c(C#N)c54)n3)n2)n1 ; CCc1c(F)ccc2cc(O)cc(-c3ncc4c(N(C)CCCC(=O)NC(C)=O)nc(OC[C@@]56CCCN5C[C@H](F)C6)nc4c3F)c12]
and pharmaceutically acceptable salts thereof.

40. A pharmaceutical composition, comprising a therapeutically effective amount of a compound of claim 1 or a pharmaceutically acceptable salt thereof, and a pharmaceutically acceptable excipient.

41. A method for inhibiting KRas G12S and/or KRas G12C activity in a cell, comprising contacting the cell in which inhibition of KRas G12S and/or KRas G12C activity is desired with an effective amount of a compound of according to claim 1 or a pharmaceutically acceptable salt thereof.

42. A method for treating cancer comprising administering to a patient having cancer a therapeutically effective amount of a compound according to claim 1 or a pharmaceutically acceptable salt thereof.

43. The method of claim 42, wherein the therapeutically effective amount of the compound is between about 0.01 to 100 mg/kg per day.

44. (canceled)

45. The method of claim 42, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

46. The method of claim 42, wherein the cancer is a KRas G12S-associated cancer.

47. The method of claim 42, wherein the cancer is a KRas G12C-associated cancer.

48. The method of claim 46, wherein the cancer is non-small cell lung cancer, small cell lung cancer, colorectal cancer, rectal cancer or pancreatic cancer.

49. (canceled)
